(19) United States
(12) Patent Application Publication
(10) Pub. No.: US 2025/0382617 A1
(43) Pub. Date: Dec. 18, 2025

(54) DOUBLE STRANDED OLIGONUCLEOTIDE COMPOSITIONS FOR RNA INTERFERENCE AND METHODS RELATING THERETO

(71) Applicant: WAVE LIFE SCIENCES LTD., Singapore (SG)

(72) Inventors: Chandra VARGEESE, Cambridge, MA (US); Naoki Iwamoto, Cambridge, MA (US); Wei Liu, Cambridge, MA (US); Khoa Ngoc Dang Luu, Cambridge, MA (US); Pachamuthu Kandasamy, Cambridge, MA (US); Subramanian Marappan, Cambridge, MA (US); Jayakanthan Kumarasamy, Cambridge, MA (US); Himali Kavin Shah, Cambridge, NH (US); Arindom Chatterjee, Cambridge, MA (US); Fengjiao Zhang, Cambridge, MA (US); Anthony Lamattina, Cambridge, MA (US); Fangjun Liu, Cambridge, MA (US); Jeonghoon Choi, Cambridge, MA (US); Nayantara Kothari, Cambridge, MA (US); Kuldeep Singh, Cambridge, MA (US)

(73) Assignee: WAVE LIFE SCIENCES LTD., Singapore (SG)

(21) Appl. No.: 19/314,882

(22) Filed: Aug. 29, 2025

Related U.S. Application Data
(63) Continuation of application No. PCT/US24/18169, filed on Mar. 1, 2024.
(60) Provisional application No. 63/487,823, filed on Mar. 1, 2023.
(60) Provisional application No. 63/491,720, filed on Mar. 22, 2023.
(60) Provisional application No. 63/581,614, filed on Sep. 8, 2023.
(60) Provisional application No. 63/585,133, filed on Sep. 25, 2023.

(51) Int. Cl. C12N 15/113

(52) U.S. Cl. CPC C12N 15/113; C12N 15/1137; C12Y 115/01001; C12N 2310/14; C12N 2310/351

(57) ABSTRACT

The present disclosure provides double stranded oligonucleotides, compositions, and methods relating thereto. The present disclosure encompasses the recognition that structural elements of double stranded oligonucleotides, such as base sequence, chemical modifications (e.g., modifications of sugar, base, and/or internucleotidic linkages) or patterns thereof, and/or stereochemistry (e.g., stereochemistry of backbone chiral centers (chiral internucleotidic linkages), and/or patterns thereof, can have significant impact on oligonucleotide properties and activities, e.g., RNA interference (RNAi) activity, Ago2 loading, thermal stability, in vivo stability, delivery to tissues and into cells, etc. The present disclosure also provides methods for treatment of diseases, e.g., hepatic diseases, central nervous system (CNS) diseases, etc., using provided double stranded oligonucleotide compositions, for example, in RNA interference.

### CROSS-REFERENCE TO RELATED APPLICATIONS

This application is a continuation of International Application No. PCT/US2024/018169, filed Mar. 1, 2024, which claims the benefit of U.S. Provisional Application No. 63/487,823, filed Mar. 1, 2023, U.S. Provisional Application No. 63/491,720, filed Mar. 22, 2023, U.S. Provisional Application No. 63/581,614, filed Sep. 8, 2023, and U.S. Provisional Application No. 63/585,133, filed Sep. 25, 2023, the contents of which are incorporated herein by reference in their entirety.

### SEQUENCE LISTING

The present application contains a Sequence Listing which has been submitted via EFS-Web and is hereby incorporated by reference in its entirety. Said Sequence Listing, created on Aug. 29, 2025, is named 0882900204.xml and is 15,786,610 bytes in size.

### BACKGROUND

Gene-targeting oligonucleotides are useful in various applications, e.g., therapeutic, diagnostic, research and nanomaterials applications. The use of naturally-occurring nucleic acids (e.g., unmodified DNA or RNA) in such applications can be limited by, for example, their susceptibility to endo- and exo-nucleases. As such, various synthetic counterparts have been developed to circumvent these shortcomings. These include synthetic oligonucleotides that contain chemical modifications, e.g., base modifications, sugar modifications, backbone modifications. There remains, however, a need in the art for double-stranded (ds) oligonucleotides with improved properties for use in connection with the above-described applications.

### SUMMARY

The present disclosure is directed, in part, to the recognition that controlling structural elements of the oligonucleotides of a double-stranded (ds) oligonucleotide can have a significant impact on the ds oligonucleotide's properties and/or activity. In certain embodiments, such structural elements include one or more of: (1) chemical modifications (e.g., modifications of a sugar, base and/or internucleotidic linkage) and patterns thereof; and (2) alterations in stereochemistry (e.g., stereochemistry of a backbone chiral internucleotidic linkage) and patterns thereof. One or more of such structural elements can, in certain embodiments, be independently present in one or both oligonucleotides of a ds oligonucleotide. In certain embodiments, the properties and/or activities impacted by such structural elements include, but are not limited to, participation in, direction of a decrease in expression, activity or level of a gene or a gene product thereof, mediated, for example, by RNA interference (RNAi interference), RNase H-mediated knockdown, steric hindrance of translation, etc. Moreover, in certain embodiments, the properties and/or activities impacted by such structural elements include, but are not limited to, participation in, Ago2 loading, thermal stability, in vivo stability, delivery to tissues and into cells, among others.

In certain embodiments, the present disclosure demonstrates that compositions comprising ds oligonucleotides (e.g., dsRNAi oligonucleotides, also referred to as dsRNAi agents) with controlled structural elements provide unexpected properties and/or activities.

In certain embodiments, the present disclosure encompasses the recognition that stereochemistry, e.g., stereochemistry of backbone chiral centers, can unexpectedly maintain or improve properties of ds oligonucleotides. For example, but not by way of limitation, the instant disclosure relates, in part, to ds oligonucleotides comprising a guide strand comprising backbone phosphoryl guanidine chiral centers in the Sp configuration.

In certain embodiments, the guide strand comprises a backbone phosphoryl guanidine chiral center in the Sp configuration between the +3 nucleotide and the immediately downstream (+4) nucleotide, i.e., in the 3′ direction. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, further comprise one or a plurality of modified sugars.

In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the 5′ terminal (+1) nucleotide, second (+2) nucleotide, third (+3) nucleotide, fourth (+4) nucleotide, fifth (+5) nucleotide, sixth (+6) nucleotide, seventh (+7) nucleotide, eighth (+8) nucleotide, ninth (+9) nucleotide, tenth (+10) nucleotide, twelfth (+12) nucleotide, sixteenth (+16) nucleotide, seventeenth (+17) nucleotide, eighteenth (+18) nucleotide, nineteenth (+19) nucleotide, twentieth (+20) nucleotide, twenty-first (+21) nucleotide, twenty-second (+22), and/or twenty-third (+23), the 3′ terminal, nucleotide.

In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the 5′ terminal (+1) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified at the second (+2) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the third (+3) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the fourth (+4) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the fifth (+5) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar of the sixth (+6) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar of the seventh (+7) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar of the eighth (+8) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar of the ninth (+9) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar of the tenth (+10) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar of the twelfth (+12) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar of the sixteenth (+16) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar of the seventeenth (+17) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar of the eighteenth (+18) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar of the nineteenth (+19) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar of the twentieth (+20) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar of the twenty-first (+21) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar of the twenty-second (+22) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar of the twenty-third (+23), the 3′ terminal, nucleotide.

In certain embodiments, the modified sugar comprises a 2′-F modification, 2′-H modification, 2′-OH modification, 2′-O-alkyl modification, e.g., 2′-O-methyl (OMe) modification, 2′-methoxyethyl (MOE) modification, 5′-alkyl modification, e.g., 5′-(R)-methyl or 5′-(S)-methyl, DNA, locked nucleic acid (LNA), unlocked nucleic acid (UNA), glycol nucleic acid (GNA), or Homo-DNA.

In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the 5′ terminal (+1) nucleotide, second (+2) nucleotide, third (+3) nucleotide, fourth (+4) nucleotide, fifth (+5) nucleotide, sixth (+6) nucleotide, seventh (+7) nucleotide, eighth (+8) nucleotide, ninth (+9) nucleotide, tenth (+10) nucleotide, twelfth (+12) nucleotide, sixteenth (+16) nucleotide, seventeenth (+17) nucleotide, eighteenth (+18) nucleotide, nineteenth (+19) nucleotide, twentieth (+20) nucleotide, twenty-first (+21) nucleotide, twenty-second (+22), and/or twenty-third (+23), the 3′ terminal, nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the 5′ terminal (+1) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the second (+2) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the third (+3) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the fourth (+4) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the fifth (+5) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the sixth (+6) nucleotide.

In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a Homo-DNA modification of the 5′ terminal (+1) nucleotide, sixth (+6) nucleotide, ninth (+9) nucleotide, tenth (+10) nucleotide, twelfth (+12) nucleotide, sixteenth (+16) nucleotide, seventeenth (+17) nucleotide, eighteenth (+18) nucleotide, nineteenth (+19) nucleotide, twentieth (+20) nucleotide, twenty-first (+21) nucleotide, twenty-second (+22), and/or twenty-third (+23), the 3′ terminal, nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a Homo-DNA modification of the 5′ terminal (+1) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a Homo-DNA modification of the sixth (+6) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a Homo-DNA modification of the ninth (+9) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a Homo-DNA modification of the tenth (+10) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a Homo-DNA modification of the twelfth (+12) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a Homo-DNA modification of the sixteenth (+16) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a Homo-DNA modification of the seventeenth (+17) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a Homo-DNA modification of the eighteenth (+18) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a Homo-DNA modification of the nineteenth (+19) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a Homo-DNA modification of the twentieth (+20) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a Homo-DNA modification of the twenty-first (+21) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a Homo-DNA modification of the twenty-second (+22) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a Homo-DNA modification of the twenty-third (+23), the 3′ terminal, nucleotide.

In certain embodiments, the present disclosure provides a ds oligonucleotide, useful for any purpose, which operates through any mechanism, and which comprises comprising a guide strand backbone phosphoryl guanidine chiral center in the Sp configuration between the +3 nucleotide and the immediately downstream (+4) nucleotide, i.e., in the 3′ direction, which further comprises:
(1) a guide strand comprising backbone phosphorothioate chiral centers in Sp configuration between the 3′ terminal nucleotide and the penultimate (N-1) nucleotide and as between the penultimate (N-1) nucleotide and the immediately upstream, i.e., in the 5′ direction, (N-2) nucleotide;
(2) a guide strand comprising backbone phosphorothioate chiral centers in Rp, Sp, or alternating configurations between the 5′ terminal (+1) nucleotide and the immediately downstream, i.e., in the 3′ direction, (+2) nucleotide and between the +2 nucleotide and the immediately downstream (+3) nucleotide;
(3) a guide strand comprising one or more backbone phosphorothioate chiral centers upstream, i.e., in the 5′ direction, relative to backbone phosphorothioate chiral centers in Sp configuration between the 3′ terminal nucleotide and the penultimate (N-1) nucleotide and as between the penultimate (N-1) nucleotide and the immediately upstream (N-2) nucleotide, where the upstream backbone phosphorothioate chiral centers are in Rp or Sp configuration;
(4) a guide strand comprising one or more backbone phosphorothioate chiral centers in Rp or Sp configuration between the 5′ terminal (+1) nucleotide and the immediately downstream (+2) nucleotide; and between the +2 nucleotide and the immediately downstream (+3) nucleotide;
(5) a guide strand comprising one or more backbone phosphorothioate chiral centers in Rp or Sp configuration where linkage occurs between any two adjacent nucleotides between the penultimate 3′ nucleotide of the guide strand, where N is the 3′ terminal nucleotide, and the upstream N-10 nucleotide;
(6) a guide strand comprising one or more backbone phosphoryl guanidine chiral centers in the Sp configuration between the +7 and the immediately downstream (+8), i.e., in the 3′ direction.
(7) a guide strand comprising a 5′ terminal modification;
(8) a passenger strand in combination with one or more of the aforementioned guide strands, comprising one or more backbone chiral centers in Rp or Sp configuration; (9) a passenger strand in combination with one or more of the aforementioned guide strands, comprising one or more backbone phosphoryl guanidine chiral centers in the Rp configuration between the +7 nucleotide and the immediately downstream (+8) nucleotide, i.e., in the 3′ direction;
(10) a passenger strand in combination with one or more of the aforementioned guide strands, comprising one or more backbone phosphoryl guanidine chiral centers in the Rp configuration between the +15 nucleotide and the immediately downstream (+16) nucleotide, i.e., in the 3′ direction; and
(11) a passenger strand in combination with one or more of the aforementioned guide strands, comprising backbone phosphorothioate chiral centers in the Sp configuration between the 5′ terminal (+1) nucleotide and the immediately downstream, i.e., in the 3′ direction, (+2) nucleotide and between the 3′ terminal nucleotide and the penultimate (N-1) nucleotide;
   wherein the ds oligonucleotide further comprises one or more of.
(1) a guide strand where one or both of the 5′ and 3′ terminal dinucleotides are not linked by non-negatively charged internucleotidic linkages, i.e., the guide strand comprises one more Rp, Sp, or stereorandom non-negatively charged internucleotidic linkages downstream, i.e., in the 3′ direction, relative to the linkage between the 5′ terminal dinucleotide and/or upstream, i.e., in the 5′ direction, relative to the linkage between the 3′ terminal dinucleotide;
(2) a guide strand where one or more Rp, Sp, or stereorandom non-negatively charged internucleotidic linkage occurs between any two adjacent nucleotides between the second (+2) nucleotide relative to the 5′ terminal nucleotide of the guide strand and the penultimate 3′ (N-1) nucleotide of the guide strand, where N is the 3′ terminal nucleotide;
(3) a guide strand where an Rp, Sp, or stereorandom non-negatively charged internucleotidic linkage occurs between the tenth (+10) and eleventh (+11) nucleotides, relative to the 5′ terminal nucleotide;
(4) a guide strand where an Rp, Sp, or stereorandom non-negatively charged internucleotidic linkage occurs between the seventh (+7) and eighth (+8) nucleotides, relative to the 5′ terminal nucleotide;
(5) a guide strand comprising one or more backbone phosphorothioate chiral centers in Rp or Sp configuration where linkage occurs between any two adjacent nucleotides between the penultimate 3′ nucleotide of the guide strand, where N is the 3′ terminal nucleotide, and the upstream N-10 nucleotide;
(6) a passenger strand where one or more Rp, Sp, or stereorandom non-negatively charged internucleotidic linkage occurs upstream, i.e., in the 5′ direction, relative to the central nucleotide of the passenger strand;
(7) a passenger strand where one or more Rp, Sp, or stereorandom non-negatively charged internucleotidic linkage occurs downstream, i.e., in the 3′ direction, relative to the central nucleotide of the passenger strand;
(8) a passenger strand where one or more backbone phosphorothioate chiral centers in Rp or Sp configuration internucleotidic linkage occurs upstream, i.e., in the 5′ direction, relative to the central nucleotide of the passenger strand;
(9) a passenger strand where one or more backbone phosphorothioate chiral centers in Rp or Sp configuration internucleotidic linkage internucleotidic linkage occurs downstream, i.e., in the 3′ direction, relative to the central nucleotide of the passenger strand; and
(10) a passenger strand comprising one or more modified sugars, e.g. Homo-DNA, between the 5′ terminal (+1) nucleotide and the penultimate (N-1) nucleotide.

In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a LNA modification of the 5′ terminal (+1) nucleotide, second (+2) nucleotide, third (+3) nucleotide, fourth (+4) nucleotide, fifth (+5) nucleotide, sixth (+6) nucleotide, seventh (+7) nucleotide, and/or eighth (+8) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a LNA modification of the 5′ terminal (+1) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a LNA modification of the second (+2) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a LNA modification of the third (+3) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a LNA modification of the fourth (+4) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a LNA modification of the fifth (+5) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a LNA modification of the sixth (+6) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a LNA modification of the seventh (+7) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a LNA modification of the eighth (+8) nucleotide. In certain embodiments, the present disclosure provides a ds oligonucleotide, useful for any purpose, which operates through any mechanism, and which comprises comprising a guide strand backbone phosphoryl guanidine chiral center in the Sp configuration between the +3 nucleotide and the immediately downstream (+4) nucleotide, i.e., in the 3′ direction, which further comprises:
(1) a guide strand comprising backbone phosphorothioate chiral centers in Sp configuration between the 3′ terminal nucleotide and the penultimate (N-1) nucleotide and as between the penultimate (N-1) nucleotide and the immediately upstream, i.e., in the 5′ direction, (N-2) nucleotide;
(2) a guide strand comprising backbone phosphorothioate chiral centers in Rp, Sp, or alternating configurations between the 5′ terminal (+1) nucleotide and the immediately downstream, i.e., in the 3′ direction, (+2) nucleotide and between the +2 nucleotide and the immediately downstream (+3) nucleotide;
(3) a guide strand comprising one or more backbone phosphorothioate chiral centers upstream, i.e., in the 5′ direction, relative to backbone phosphorothioate chiral centers in Sp configuration between the 3′ terminal nucleotide and the penultimate (N-1) nucleotide and as between the penultimate (N-1) nucleotide and the immediately upstream (N-2) nucleotide, where the upstream backbone phosphorothioate chiral centers are in Rp or Sp configuration;
(4) a guide strand comprising one or more backbone phosphorothioate chiral centers in Rp or Sp configuration between the 5′ terminal (+1) nucleotide and the immediately downstream (+2) nucleotide; and between the +2 nucleotide and the immediately downstream (+3) nucleotide;
(5) a guide strand comprising one or more backbone phosphorothioate chiral centers in Rp or Sp configuration where linkage occurs between any two adjacent nucleotides between the penultimate 3′ nucleotide of the guide strand, where N is the 3′ terminal nucleotide, and the upstream N-10 nucleotide;
(6) a guide strand comprising one or more backbone phosphoryl guanidine chiral centers in the Sp configuration between the +7 and the immediately downstream (+8), i.e., in the 3′ direction.
(7) a guide strand comprising a 5′ terminal modification;
(8) a passenger strand in combination with one or more of the aforementioned guide strands, comprising one or more backbone chiral centers in Rp or Sp configuration;
(9) a passenger strand in combination with one or more of the aforementioned guide strands, comprising one or more backbone phosphoryl guanidine chiral centers in the Rp configuration between the +7 nucleotide and the immediately downstream (+8) nucleotide, i.e., in the 3′ direction;
(10) a passenger strand in combination with one or more of the aforementioned guide strands, comprising one or more backbone phosphoryl guanidine chiral centers in the Rp configuration between the +15 nucleotide and the immediately downstream (+16) nucleotide, i.e., in the 3′ direction; and
(11) a passenger strand in combination with one or more of the aforementioned guide strands, comprising backbone phosphorothioate chiral centers in the Sp configuration between the 5′ terminal (+1) nucleotide and the immediately downstream, i.e., in the 3′ direction, (+2) nucleotide and between the 3′ terminal nucleotide and the penultimate (N-1) nucleotide;
   wherein the ds oligonucleotide further comprises one or more of.
(1) a guide strand where one or both of the 5′ and 3′ terminal dinucleotides are not linked by non-negatively charged internucleotidic linkages, i.e., the guide strand comprises one more Rp, Sp, or stereorandom non-negatively charged internucleotidic linkages downstream, i.e., in the 3′ direction, relative to the linkage between the 5′ terminal dinucleotide and/or upstream, i.e., in the 5′ direction, relative to the linkage between the 3′ terminal dinucleotide;
(2) a guide strand where one or more Rp, Sp, or stereorandom non-negatively charged internucleotidic linkage occurs between any two adjacent nucleotides between the second (+2) nucleotide relative to the 5′ terminal nucleotide of the guide strand and the penultimate 3′ (N-1) nucleotide of the guide strand, where N is the 3′ terminal nucleotide;
(3) a guide strand where an Rp, Sp, or stereorandom non-negatively charged internucleotidic linkage occurs between the tenth (+10) and eleventh (+11) nucleotides, relative to the 5′ terminal nucleotide;
(4) a guide strand where an Rp, Sp, or stereorandom non-negatively charged internucleotidic linkage occurs between the seventh (+7) and eighth (+8) nucleotides, relative to the 5′ terminal nucleotide;
(5) a guide strand comprising one or more backbone phosphorothioate chiral centers in Rp or Sp configuration where linkage occurs between any two adjacent nucleotides between the penultimate 3′ nucleotide of the guide strand, where N is the 3′ terminal nucleotide, and the upstream N-10 nucleotide;
(6) a passenger strand where one or more Rp, Sp, or stereorandom non-negatively charged internucleotidic linkage occurs upstream, i.e., in the 5′ direction, relative to the central nucleotide of the passenger strand;
(7) a passenger strand where one or more Rp, Sp, or stereorandom non-negatively charged internucleotidic linkage occurs downstream, i.e., in the 3′ direction, relative to the central nucleotide of the passenger strand;
(8) a passenger strand where one or more backbone phosphorothioate chiral centers in Rp or Sp configuration internucleotidic linkage occurs upstream, i.e., in the 5′ direction, relative to the central nucleotide of the passenger strand;
(9) a passenger strand where one or more backbone phosphorothioate chiral centers in Rp or Sp configuration internucleotidic linkage internucleotidic linkage occurs downstream, i.e., in the 3′ direction, relative to the central nucleotide of the passenger strand; and
(10) a passenger strand comprising one or more modified sugars, e.g. Homo-DNA, between the 5′ terminal (+1) nucleotide and the penultimate (N-1) nucleotide.

In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-O-alkyl modification of the 5′ terminal (+1) nucleotide, second (+2) nucleotide, third (+3) nucleotide, fourth (+4) nucleotide, fifth (+5) nucleotide, sixth (+6) nucleotide, seventh (+7) nucleotide, and/or eighth (+8) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-O-alkyl modification of the 5′ terminal (+1) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-O-alkyl modification of the second (+2) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-O-alkyl modification of the third (+3) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-O-alkyl modification of the fourth (+4) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-O-alkyl modification of the fifth (+5) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-O-alkyl modification of the sixth (+6) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-O-alkyl modification of the seventh (+7) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-O-alkyl modification of the eighth (+8) nucleotide.

In certain embodiments, the present disclosure provides a ds oligonucleotide, useful for any purpose, which operates through any mechanism, and which comprises comprising a guide strand backbone phosphoryl guanidine chiral center in the Sp configuration between the +3 nucleotide and the immediately downstream (+4) nucleotide, i.e., in the 3′ direction, which further comprises:
(1) a guide strand comprising backbone phosphorothioate chiral centers in Sp configuration between the 3′ terminal nucleotide and the penultimate (N-1) nucleotide and as between the penultimate (N-1) nucleotide and the immediately upstream, i.e., in the 5′ direction, (N-2) nucleotide;
(2) a guide strand comprising backbone phosphorothioate chiral centers in Rp, Sp, or alternating configurations between the 5′ terminal (+1) nucleotide and the immediately downstream, i.e., in the 3′ direction, (+2) nucleotide and between the +2 nucleotide and the immediately downstream (+3) nucleotide;
(3) a guide strand comprising one or more backbone phosphorothioate chiral centers upstream, i.e., in the 5′ direction, relative to backbone phosphorothioate chiral centers in Sp configuration between the 3′ terminal nucleotide and the penultimate (N-1) nucleotide and as between the penultimate (N-1) nucleotide and the immediately upstream (N-2) nucleotide, where the upstream backbone phosphorothioate chiral centers are in Rp or Sp configuration;
(4) a guide strand comprising one or more backbone phosphorothioate chiral centers in Rp or Sp configuration between the 5′ terminal (+1) nucleotide and the immediately downstream (+2) nucleotide; and between the +2 nucleotide and the immediately downstream (+3) nucleotide;
(5) a guide strand comprising one or more backbone phosphorothioate chiral centers in Rp or Sp configuration where linkage occurs between any two adjacent nucleotides between the penultimate 3′ nucleotide of the guide strand, where N is the 3′ terminal nucleotide, and the upstream N-10 nucleotide;
(6) a guide strand comprising one or more backbone phosphoryl guanidine chiral centers in the Sp configuration between the +7 and the immediately downstream (+8), i.e., in the 3′ direction.
(7) a guide strand comprising a 5′ terminal modification;
(8) a passenger strand in combination with one or more of the aforementioned guide strands, comprising one or more backbone chiral centers in Rp or Sp configuration;
(9) a passenger strand in combination with one or more of the aforementioned guide strands, comprising one or more backbone phosphoryl guanidine chiral centers in the Rp configuration between the +7 nucleotide and the immediately downstream (+8) nucleotide, i.e., in the 3′ direction;
(10) a passenger strand in combination with one or more of the aforementioned guide strands, comprising one or more backbone phosphoryl guanidine chiral centers in the Rp configuration between the +15 nucleotide and the immediately downstream (+16) nucleotide, i.e., in the 3′ direction; and
(11) a passenger strand in combination with one or more of the aforementioned guide strands, comprising backbone phosphorothioate chiral centers in the Sp configuration between the 5′ terminal (+1) nucleotide and the immediately downstream, i.e., in the 3′ direction, (+2) nucleotide and between the 3′ terminal nucleotide and the penultimate (N-1) nucleotide;
   wherein the ds oligonucleotide further comprises one or more of.
(1) a guide strand where one or both of the 5′ and 3′ terminal dinucleotides are not linked by non-negatively charged internucleotidic linkages, i.e., the guide strand comprises one more Rp, Sp, or stereorandom non-negatively charged internucleotidic linkages downstream, i.e., in the 3′ direction, relative to the linkage between the 5′ terminal dinucleotide and/or upstream, i.e., in the 5′ direction, relative to the linkage between the 3′ terminal dinucleotide;
(2) a guide strand where one or more Rp, Sp, or stereorandom non-negatively charged internucleotidic linkage occurs between any two adjacent nucleotides between the second (+2) nucleotide relative to the 5′ terminal nucleotide of the guide strand and the penultimate 3′ (N-1) nucleotide of the guide strand, where N is the 3′ terminal nucleotide;
(3) a guide strand where an Rp, Sp, or stereorandom non-negatively charged internucleotidic linkage occurs between the tenth (+10) and eleventh (+11) nucleotides, relative to the 5′ terminal nucleotide;
(4) a guide strand where an Rp, Sp, or stereorandom non-negatively charged internucleotidic linkage occurs between the seventh (+7) and eighth (+8) nucleotides, relative to the 5′ terminal nucleotide;
(5) a guide strand comprising one or more backbone phosphorothioate chiral centers in Rp or Sp configuration where linkage occurs between any two adjacent nucleotides between the penultimate 3′ nucleotide of the guide strand, where N is the 3′ terminal nucleotide, and the upstream N-10 nucleotide;
(6) a passenger strand where one or more Rp, Sp, or stereorandom non-negatively charged internucleotidic linkage occurs upstream, i.e., in the 5′ direction, relative to the central nucleotide of the passenger strand;
(7) a passenger strand where one or more Rp, Sp, or stereorandom non-negatively charged internucleotidic linkage occurs downstream, i.e., in the 3′ direction, relative to the central nucleotide of the passenger strand;
(8) a passenger strand where one or more backbone phosphorothioate chiral centers in Rp or Sp configuration internucleotidic linkage occurs upstream, i.e., in the 5′ direction, relative to the central nucleotide of the passenger strand;
(9) a passenger strand where one or more backbone phosphorothioate chiral centers in Rp or Sp configuration internucleotidic linkage internucleotidic linkage occurs downstream, i.e., in the 3′ direction, relative to the central nucleotide of the passenger strand; and
(10) a passenger strand comprising one or more modified sugars, e.g. Homo-DNA, between the 5′ terminal (+1) nucleotide and the penultimate (N-1) nucleotide.

In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a DNA modification of the 5′ terminal (+1) nucleotide, second (+2) nucleotide, third (+3) nucleotide, fourth (+4) nucleotide, fifth (+5) nucleotide, sixth (+6) nucleotide, seventh (+7) nucleotide, and/or eighth (+8) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a DNA modification of the 5′ terminal (+1) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a DNA modification of the second (+2) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a DNA modification of the third (+3) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a DNA modification of the fourth (+4) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a DNA modification of the fifth (+5) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a DNA modification of the sixth (+6) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a DNA modification of the seventh (+7) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a DNA modification of the eighth (+8) nucleotide.

In certain embodiments, the present disclosure provides a ds oligonucleotide, useful for any purpose, which operates through any mechanism, and which comprises comprising a guide strand backbone phosphoryl guanidine chiral center in the Sp configuration between the +3 nucleotide and the immediately downstream (+4) nucleotide, i.e., in the 3′ direction, which further comprises:
(1) a guide strand comprising backbone phosphorothioate chiral centers in Sp configuration between the 3′ terminal nucleotide and the penultimate (N-1) nucleotide and as between the penultimate (N-1) nucleotide and the immediately upstream, i.e., in the 5′ direction, (N-2) nucleotide;
(2) a guide strand comprising backbone phosphorothioate chiral centers in Rp, Sp, or alternating configurations between the 5′ terminal (+1) nucleotide and the immediately downstream, i.e., in the 3′ direction, (+2) nucleotide and between the +2 nucleotide and the immediately downstream (+3) nucleotide;
(3) a guide strand comprising one or more backbone phosphorothioate chiral centers upstream, i.e., in the 5′ direction, relative to backbone phosphorothioate chiral centers in Sp configuration between the 3′ terminal nucleotide and the penultimate (N-1) nucleotide and as between the penultimate (N-1) nucleotide and the immediately upstream (N-2) nucleotide, where the upstream backbone phosphorothioate chiral centers are in Rp or Sp configuration;
(4) a guide strand comprising one or more backbone phosphorothioate chiral centers in Rp or Sp configuration between the 5′ terminal (+1) nucleotide and the immediately downstream (+2) nucleotide; and between the +2 nucleotide and the immediately downstream (+3) nucleotide;
(5) a guide strand comprising one or more backbone phosphorothioate chiral centers in Rp or Sp configuration where linkage occurs between any two adjacent nucleotides between the penultimate 3′ nucleotide of the guide strand, where N is the 3′ terminal nucleotide, and the upstream N-10 nucleotide;
(6) a guide strand comprising one or more backbone phosphoryl guanidine chiral centers in the Sp configuration between the +7 and the immediately downstream (+8), i.e., in the 3′ direction.
(7) a guide strand comprising a 5′ terminal modification;
(8) a passenger strand in combination with one or more of the aforementioned guide strands, comprising one or more backbone chiral centers in Rp or Sp configuration;
(9) a passenger strand in combination with one or more of the aforementioned guide strands, comprising one or more backbone phosphoryl guanidine chiral centers in the Rp configuration between the +7 nucleotide and the immediately downstream (+8) nucleotide, i.e., in the 3′ direction;
(10) a passenger strand in combination with one or more of the aforementioned guide strands, comprising one or more backbone phosphoryl guanidine chiral centers in the Rp configuration between the +15 nucleotide and the immediately downstream (+16) nucleotide, i.e., in the 3′ direction; and
(11) a passenger strand in combination with one or more of the aforementioned guide strands, comprising backbone phosphorothioate chiral centers in the Sp configuration between the 5′ terminal (+1) nucleotide and the immediately downstream, i.e., in the 3′ direction, (+2) nucleotide and between the 3′ terminal nucleotide and the penultimate (N-1) nucleotide;
   wherein the ds oligonucleotide further comprises one or more of:
(1) a guide strand where one or both of the 5′ and 3′ terminal dinucleotides are not linked by non-negatively charged internucleotidic linkages, i.e., the guide strand comprises one more Rp, Sp, or stereorandom non-negatively charged internucleotidic linkages downstream, i.e., in the 3′ direction, relative to the linkage between the 5′ terminal dinucleotide and/or upstream, i.e., in the 5′ direction, relative to the linkage between the 3′ terminal dinucleotide;
(2) a guide strand where one or more Rp, Sp, or stereorandom non-negatively charged internucleotidic linkage occurs between any two adjacent nucleotides between the second (+2) nucleotide relative to the 5′ terminal nucleotide of the guide strand and the penultimate 3′ (N-1) nucleotide of the guide strand, where N is the 3′ terminal nucleotide;
(3) a guide strand where an Rp, Sp, or stereorandom non-negatively charged internucleotidic linkage occurs between the tenth (+10) and eleventh (+11) nucleotides, relative to the 5′ terminal nucleotide;
(4) a guide strand where an Rp, Sp, or stereorandom non-negatively charged internucleotidic linkage occurs between the seventh (+7) and eighth (+8) nucleotides, relative to the 5′ terminal nucleotide;
(5) a guide strand comprising one or more backbone phosphorothioate chiral centers in Rp or Sp configuration where linkage occurs between any two adjacent nucleotides between the penultimate 3′ nucleotide of the guide strand, where N is the 3′ terminal nucleotide, and the upstream N-10 nucleotide;
(6) a passenger strand where one or more Rp, Sp, or stereorandom non-negatively charged internucleotidic linkage occurs upstream, i.e., in the 5′ direction, relative to the central nucleotide of the passenger strand;
(7) a passenger strand where one or more Rp, Sp, or stereorandom non-negatively charged internucleotidic linkage occurs downstream, i.e., in the 3′ direction, relative to the central nucleotide of the passenger strand;
(8) a passenger strand where one or more backbone phosphorothioate chiral centers in Rp or Sp configuration internucleotidic linkage occurs upstream, i.e., in the 5′ direction, relative to the central nucleotide of the passenger strand;
(9) a passenger strand where one or more backbone phosphorothioate chiral centers in Rp or Sp configuration internucleotidic linkage internucleotidic linkage occurs downstream, i.e., in the 3′ direction, relative to the central nucleotide of the passenger strand; and
(10) a passenger strand comprising one or more modified sugars, e.g. Homo-DNA, between the 5′ terminal (+1) nucleotide and the penultimate (N-1) nucleotide.

In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a GNA modification of the 5′ terminal (+1) nucleotide, second (+2) nucleotide, third (+3) nucleotide, fourth (+4) nucleotide, fifth (+5) nucleotide, sixth (+6) nucleotide, seventh (+7) nucleotide, and/or eighth (+8) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a GNA modification of the 5′ terminal (+1) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a GNA modification of the second (+2) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a GNA modification of the third (+3) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a GNA modification of the fourth (+4) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a GNA modification of the fifth (+5) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a GNA modification of the sixth (+6) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a GNA modification of the seventh (+7) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a GNA modification of the eighth (+8) nucleotide.

In certain embodiments, the present disclosure provides a ds oligonucleotide, useful for any purpose, which operates through any mechanism, and which comprises comprising a guide strand backbone phosphoryl guanidine chiral center in the Sp configuration between the +3 nucleotide and the immediately downstream (+4) nucleotide, i.e., in the 3′ direction, which further comprises:
(1) a guide strand comprising backbone phosphorothioate chiral centers in Sp configuration between the 3′ terminal nucleotide and the penultimate (N-1) nucleotide and as between the penultimate (N-1) nucleotide and the immediately upstream, i.e., in the 5′ direction, (N-2) nucleotide;
(2) a guide strand comprising backbone phosphorothioate chiral centers in Rp, Sp, or alternating configurations between the 5′ terminal (+1) nucleotide and the immediately downstream, i.e., in the 3′ direction, (+2) nucleotide and between the +2 nucleotide and the immediately downstream (+3) nucleotide;
(3) a guide strand comprising one or more backbone phosphorothioate chiral centers upstream, i.e., in the 5′ direction, relative to backbone phosphorothioate chiral centers in Sp configuration between the 3′ terminal nucleotide and the penultimate (N-1) nucleotide and as between the penultimate (N-1) nucleotide and the immediately upstream (N-2) nucleotide, where the upstream backbone phosphorothioate chiral centers are in Rp or Sp configuration;
(4) a guide strand comprising one or more backbone phosphorothioate chiral centers in Rp or Sp configuration between the 5′ terminal (+1) nucleotide and the immediately downstream (+2) nucleotide; and between the +2 nucleotide and the immediately downstream (+3) nucleotide;
(5) a guide strand comprising one or more backbone phosphorothioate chiral centers in Rp or Sp configuration where linkage occurs between any two adjacent nucleotides between the penultimate 3′ nucleotide of the guide strand, where N is the 3′ terminal nucleotide, and the upstream N-10 nucleotide;
(6) a guide strand comprising one or more backbone phosphoryl guanidine chiral centers in the Sp configuration between the +7 and the immediately downstream (+8), i.e., in the 3′ direction.
(7) a guide strand comprising a 5′ terminal modification;
(8) a passenger strand in combination with one or more of the aforementioned guide strands, comprising one or more backbone chiral centers in Rp or Sp configuration;
(9) a passenger strand in combination with one or more of the aforementioned guide strands, comprising one or more backbone phosphoryl guanidine chiral centers in the Rp configuration between the +7 nucleotide and the immediately downstream (+8) nucleotide, i.e., in the 3′ direction;
(10) a passenger strand in combination with one or more of the aforementioned guide strands, comprising one or more backbone phosphoryl guanidine chiral centers in the Rp configuration between the +15 nucleotide and the immediately downstream (+16) nucleotide, i.e., in the 3′ direction; and
(11) a passenger strand in combination with one or more of the aforementioned guide strands, comprising backbone phosphorothioate chiral centers in the Sp configuration between the 5′ terminal (+1) nucleotide and the immediately downstream, i.e., in the 3′ direction, (+2) nucleotide and between the 3′ terminal nucleotide and the penultimate (N-1) nucleotide;
   wherein the ds oligonucleotide further comprises one or more of:
(1) a guide strand where one or both of the 5′ and 3′ terminal dinucleotides are not linked by non-negatively charged internucleotidic linkages, i.e., the guide strand comprises one more Rp, Sp, or stereorandom non-negatively charged internucleotidic linkages downstream, i.e., in the 3′ direction, relative to the linkage between the 5′ terminal dinucleotide and/or upstream, i.e., in the 5′ direction, relative to the linkage between the 3′ terminal dinucleotide;
(2) a guide strand where one or more Rp, Sp, or stereorandom non-negatively charged internucleotidic linkage occurs between any two adjacent nucleotides between the second (+2) nucleotide relative to the 5′ terminal nucleotide of the guide strand and the penultimate 3′ (N-1) nucleotide of the guide strand, where N is the 3′ terminal nucleotide;
(3) a guide strand where an Rp, Sp, or stereorandom non-negatively charged internucleotidic linkage occurs between the tenth (+10) and eleventh (+11) nucleotides, relative to the 5′ terminal nucleotide;
(4) a guide strand where an Rp, Sp, or stereorandom non-negatively charged internucleotidic linkage occurs between the seventh (+7) and eighth (+8) nucleotides, relative to the 5′ terminal nucleotide;
(5) a guide strand comprising one or more backbone phosphorothioate chiral centers in Rp or Sp configuration where linkage occurs between any two adjacent nucleotides between the penultimate 3′ nucleotide of the guide strand, where N is the 3′ terminal nucleotide, and the upstream N-10 nucleotide;
(6) a passenger strand where one or more Rp, Sp, or stereorandom non-negatively charged internucleotidic linkage occurs upstream, i.e., in the 5′ direction, relative to the central nucleotide of the passenger strand;
(7) a passenger strand where one or more Rp, Sp, or stereorandom non-negatively charged internucleotidic linkage occurs downstream, i.e., in the 3′ direction, relative to the central nucleotide of the passenger strand;
(8) a passenger strand where one or more backbone phosphorothioate chiral centers in Rp or Sp configuration internucleotidic linkage occurs upstream, i.e., in the 5′ direction, relative to the central nucleotide of the passenger strand;
(9) a passenger strand where one or more backbone phosphorothioate chiral centers in Rp or Sp configuration internucleotidic linkage internucleotidic linkage occurs downstream, i.e., in the 3′ direction, relative to the central nucleotide of the passenger strand; and
(10) a passenger strand comprising one or more modified sugars, e.g. Homo-DNA, between the 5′ terminal (+1) nucleotide and the penultimate (N-1) nucleotide.

In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a (s)-GNA modification of the 5′ terminal (+1) nucleotide, second (+2) nucleotide, third (+3) nucleotide, fourth (+4) nucleotide, fifth (+5) nucleotide, sixth (+6) nucleotide, seventh (+7) nucleotide, and/or eighth (+8) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a (s)-GNA modification of the 5′ terminal (+1) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a (s)-GNA modification of the second (+2) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a (s)-GNA modification of the third (+3) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a (s)-GNA modification of the fourth (+4) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a (s)-GNA modification of the fifth (+5) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a (s)-GNA modification of the sixth (+6) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a (s)-GNA modification of the seventh (+7) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a (s)-GNA modification of the eighth (+8) nucleotide.

In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, further comprises one or more of.
(1) a guide strand comprising backbone phosphorothioate chiral centers in Sp configuration between the 3′ terminal nucleotide and the penultimate (N-1) nucleotide and as between the penultimate (N-1) nucleotide and the immediately upstream, i.e., in the 5′ direction, (N-2) nucleotide;
(2) a guide strand comprising backbone phosphorothioate chiral centers in Rp, Sp, or alternating configurations between the 5′ terminal (+1) nucleotide and the immediately downstream, i.e., in the 3′ direction, (+2) nucleotide and between the +2 nucleotide and the immediately downstream (+3) nucleotide;
(3) a guide strand comprising one or more backbone phosphorothioate chiral centers upstream, i.e., in the 5′ direction, relative to backbone phosphorothioate chiral centers in Sp configuration between the 3′ terminal nucleotide and the penultimate (N-1) nucleotide and as between the penultimate (N-1) nucleotide and the immediately upstream (N-2) nucleotide, where the upstream backbone phosphorothioate chiral centers are in Rp or Sp configuration;
(4) a guide strand comprising one or more backbone phosphorothioate chiral centers in Rp or Sp configuration between the 5′ terminal (+1) nucleotide and the immediately downstream (+2) nucleotide; and between the +2 nucleotide and the immediately downstream (+3) nucleotide;
(5) a guide strand comprising one or more backbone phosphorothioate chiral centers in Rp or Sp configuration where linkage occurs between any two adjacent nucleotides between the penultimate 3′ nucleotide of the guide strand, where N is the 3′ terminal nucleotide, and the upstream N-10 nucleotide;
(6) a guide strand comprising one or more backbone phosphoryl guanidine chiral centers in the Sp configuration between the +7 and the immediately downstream (+8), i.e., in the 3′ direction.
(7) a guide strand comprising a 5′ terminal modification;
(8) a passenger strand in combination with one or more of the aforementioned guide strands, comprising one or more backbone chiral centers in Rp or Sp configuration;
(9) a passenger strand in combination with one or more of the aforementioned guide strands, comprising one or more backbone phosphoryl guanidine chiral centers in the Rp configuration between the +7 nucleotide and the immediately downstream (+8) nucleotide, i.e., in the 3′ direction;
(10) a passenger strand in combination with one or more of the aforementioned guide strands, comprising one or more backbone phosphoryl guanidine chiral centers in the Rp configuration between the +15 nucleotide and the immediately downstream (+16) nucleotide, i.e., in the 3′ direction; and
(11) a passenger strand in combination with one or more of the aforementioned guide strands, comprising backbone phosphorothioate chiral centers in the Sp configuration between the 5′ terminal (+1) nucleotide and the immediately downstream, i.e., in the 3′ direction, (+2) nucleotide and between the 3′ terminal nucleotide and the penultimate (N-1) nucleotide;
   wherein the ds oligonucleotide further comprises one or more of.
(1) a guide strand where one or both of the 5′ and 3′ terminal dinucleotides are not linked by a Rp, Sp, or stereorandom non-negatively charged internucleotidic linkages, i.e., the guide strand comprises one or more Rp, Sp, or stereorandom non-negatively charged internucleotidic linkages downstream, i.e., in the 3′ direction, relative to the linkage between the 5′ terminal dinucleotide and/or upstream, i.e., in the 5′ direction, relative to the linkage between the 3′ terminal dinucleotide;
(2) a guide strand where one or more Rp, Sp, or stereorandom non-negatively charged internucleotidic linkage occurs between any two adjacent nucleotides between the second (+2) nucleotide relative to the 5′ terminal nucleotide of the guide strand and the penultimate 3′ (N-1) nucleotide of the guide strand, where N is the 3′ terminal nucleotide;
(3) a guide strand where an Rp, Sp, or stereorandom non-negatively charged internucleotidic linkage occurs between the tenth (+10) and eleventh (+11) nucleotides, relative to the 5′ terminal nucleotide;
(4) a guide strand where an Rp, Sp, or stereorandom non-negatively charged internucleotidic linkage occurs between the seventh (+7) and eighth (+8) nucleotides, relative to the 5′ terminal nucleotide;
(5) a guide strand comprising one or more backbone phosphorothioate chiral centers in Rp or Sp configuration where linkage occurs between any two adjacent nucleotides between the penultimate 3′ nucleotide of the guide strand, where N is the 3′ terminal nucleotide, and the upstream N-10 nucleotide;
(6) a passenger strand where one or more Rp, Sp, or stereorandom non-negatively charged internucleotidic linkage occurs upstream, i.e., in the 5′ direction, relative to the central nucleotide of the passenger strand; and
(7) a passenger strand where one or more Rp, Sp, or stereorandom non-negatively charged internucleotidic linkage occurs downstream, i.e., in the 3′ direction, relative to the central nucleotide of the passenger strand.
(8) a passenger strand where one or more backbone phosphorothioate chiral centers in Rp or Sp configuration internucleotidic linkage occurs upstream, i.e., in the 5′ direction, relative to the central nucleotide of the passenger strand;
(9) a passenger strand where one or more backbone phosphorothioate chiral centers in Rp or Sp configuration internucleotidic linkage internucleotidic linkage occurs downstream, i.e., in the 3′ direction, relative to the central nucleotide of the passenger strand; and
(10) a passenger strand comprising one or more modified sugars, e.g. Homo-DNA, between the 5′ terminal (+1) nucleotide and the penultimate (N-1) nucleotide.
   In certain embodiments, the ds oligonucleotide further comprises a 2′ modification, e.g., a 2′ F modification, of the 3′ nucleotide of a nucleotide pair linked by a Rp, Sp, or stereorandom non-negatively charged internucleotidic linkage. In certain embodiments, the one or more Rp, Sp, or stereorandom non-negatively charged internucleotidic linkage incorporated into the guide or passenger strand is an Rp non-negatively charged internucleotidic linkage. In certain embodiments, the one or more Rp, Sp, or stereorandom non-negatively charged internucleotidic linkage is an Sp non-negatively charged internucleotidic linkage. In certain embodiments, the one or more Rp, Sp, or stereorandom non-negatively charged internucleotidic linkage is a stereorandom non-negatively charged internucleotidic linkage.

In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, further comprise one or a plurality of modified sugars. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the 5′ terminal (+1) nucleotide, second (+2) nucleotide, third (+3) nucleotide, fourth (+4) nucleotide, fifth (+5) nucleotide, sixth (+6) nucleotide, seventh (+7) nucleotide, eighth (+8), ninth (+9) nucleotide, tenth (+10) nucleotide, twelfth (+12) nucleotide, sixteenth (+16) nucleotide, seventeenth (+17) nucleotide, eighteenth (+18) nucleotide, nineteenth (+19) nucleotide, twentieth (+20) nucleotide, twenty-first (+21) nucleotide, twenty-second (+22), and/or twenty-third (+23), the 3′ terminal, nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the 5′ terminal (+1) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the second (+2) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the third (+3) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the fourth (+4) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the fifth (+5) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the sixth (+6) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar of the seventh (+7) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar of the eighth (+8) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar of the ninth (+9) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar of the tenth (+10) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar of the twelfth (+12) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar of the sixteenth (+16) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar of the seventeenth (+17) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar of the eighteenth (+18) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar of the nineteenth (+19) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar of the twentieth (+20) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar of the twenty-first (+21) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar of the twenty-second (+22) nucleotide. In certain embodiments, the ds oligonucleotides comprising a guide strand backbone phosphoryl guanidine chiral center in the Sp configuration between the +3 nucleotide and the immediately downstream (+4)nucleotide, i.e., in the 3′ direction, comprise a modified sugar of the twenty-third (+23), the 3′ terminal, nucleotide.

In certain embodiments, the modified sugar comprises a 2′-F modification, 2′-H modification, 2′-OH modification, 2′-O-alkyl modification, e.g., 2′-O-methyl (OMe) modification, 2′-methoxyethyl (MOE) modification, 5′-alkyl modification, e.g., 5′-(R)-methyl or 5′-(S)-methyl, DNA, locked nucleic acid (LNA), unlocked nucleic acid (UNA), glycol nucleic acid (GNA), or Homo-DNA.

In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise one or a plurality of a 2′-F modifications. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the 5′ terminal (+1) nucleotide, second (+2) nucleotide, third (+3) nucleotide, fourth (+4) nucleotide, fifth (+5) nucleotide, and/or sixth (+6) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the 5′ terminal (+1) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the second (+2) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the third (+3) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the fourth (+4) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the fifth (+5) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the sixth (+6) nucleotide.

In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise one or a plurality of Homo-DNA modifications. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a Homo-DNA modification of the 5′ terminal (+1) nucleotide, sixth (+6) nucleotide, ninth (+9) nucleotide, tenth (+10) nucleotide, twelfth (+12)nucleotide, sixteenth (+16) nucleotide, seventeenth (+17) nucleotide, eighteenth (+18) nucleotide, nineteenth (+19) nucleotide, twentieth (+20) nucleotide, twenty-first (+21) nucleotide, twenty-second (+22), and/or twenty-third (+23), the 3′ terminal, nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a Homo-DNA modification of the 5′ terminal (+1) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a Homo-DNA modification of the sixth (+6) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a Homo-DNA modification of the ninth (+9) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a Homo-DNA modification of the tenth (+10) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a Homo-DNA modification of the twelfth (+12) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a Homo-DNA modification of the sixteenth (+16) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a Homo-DNA modification of the seventeenth (+17) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a Homo-DNA modification of the eighteenth (+18) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a Homo-DNA modification of the nineteenth (+19) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a Homo-DNA modification of the twentieth (+20) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a Homo-DNA modification of the twenty-first (+21) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a Homo-DNA modification of the twenty-second (+22) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a Homo-DNA modification of the twenty-third (+23), the 3′ terminal, nucleotide.

In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 5′-alkyl modification of the third (+3) nucleotide and/or fourth (+4) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 5′-methyl modification of the third (+3) nucleotide and/or fourth (+4) nucleotide In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 5′-alkyl modification of the third (+3) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 5′-methyl modification of the third (+3) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 5′-alkyl modification of the fourth (+4) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 5′-methyl modification of the fourth (+4) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 5′-alkyl modification of the third (+3) nucleotide and fourth (+4) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 5′-methyl modification of the third (+3) nucleotide and fourth (+4) nucleotide

In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise one or a plurality of LNA modifications. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise one or a plurality of LNA modifications. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a LNA modification of the 5′ terminal (+1) nucleotide, second (+2) nucleotide, third (+3) nucleotide, fourth (+4) nucleotide, fifth (+5) nucleotide, sixth (+6) nucleotide, seventh (+7) nucleotide, and/or eighth (+8) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a LNA modification of the 5′ terminal (+1) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a LNA modification of the second (+2) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a LNA modification of the third (+3) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a LNA modification of the fourth (+4) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a LNA modification of the fifth (+5) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a LNA modification of the sixth (+6) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a LNA modification of the seventh (+7) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a LNA modification of the eighth (+8) nucleotide.

In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise one or a plurality of 2′-o-alkyl modifications. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-O-alkyl modification of the 5′ terminal (+1) nucleotide, second (+2) nucleotide, third (+3) nucleotide, fourth (+4) nucleotide, fifth (+5) nucleotide, sixth (+6) nucleotide, seventh (+7) nucleotide, and/or eighth (+8) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-O-alkyl modification of the 5′ terminal (+1) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-O-alkyl modification of the second (+2) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-O-alkyl modification of the third (+3) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-O-alkyl modification of the fourth (+4) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-O-alkyl modification of the fifth (+5) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-O-alkyl modification of the sixth (+6) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-O-alkyl modification of the seventh (+7) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-O-alkyl modification of the eighth (+8) nucleotide.

In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise one or a plurality of DNA modifications. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a DNA modification of the 5′ terminal (+1) nucleotide, second (+2) nucleotide, third (+3) nucleotide, fourth (+4) nucleotide, fifth (+5) nucleotide, sixth (+6) nucleotide, seventh (+7) nucleotide, and/or eighth (+8) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a DNA modification of the 5′ terminal (+1) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a DNA modification of the second (+2) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a DNA modification of the third (+3) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a DNA modification of the fourth (+4) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a DNA modification of the fifth (+5) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a DNA modification of the sixth (+6) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a DNA modification of the seventh (+7) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a DNA modification of the eighth (+8) nucleotide.

In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise one or a plurality of GNA modifications. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a GNA modification of the 5′ terminal (+1) nucleotide, second (+2) nucleotide, third (+3) nucleotide, fourth (+4) nucleotide, fifth (+5) nucleotide, sixth (+6) nucleotide, seventh (+7) nucleotide, and/or eighth (+8) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a GNA modification of the 5′ terminal (+1) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a GNA modification of the second (+2) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a GNA modification of the third (+3) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a GNA modification of the fourth (+4) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a GNA modification of the fifth (+5) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a GNA modification of the sixth (+6) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a GNA modification of the seventh (+7) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a GNA modification of the eighth (+8) nucleotide.

In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise one or a plurality of (s)-GNA modifications. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a (s)-GNA modification of the 5′ terminal (+1) nucleotide, second (+2) nucleotide, third (+3) nucleotide, fourth (+4) nucleotide, fifth (+5) nucleotide, sixth (+6) nucleotide, seventh (+7) nucleotide, and/or eighth (+8) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a (s)-GNA modification of the 5′ terminal (+1) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a (s)-GNA modification of the second (+2) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a (s)-GNA modification of the third (+3) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a (s)-GNA modification of the fourth (+4) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a (s)-GNA modification of the fifth (+5) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a (s)-GNA modification of the sixth (+6) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a (s)-GNA modification of the seventh (+7) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a (s)-GNA modification of the eighth (+8) nucleotide.

In certain embodiments, the present disclosure encompasses the recognition that stereochemistry, e.g., stereochemistry of chiral centers at a 5′ terminal modification of guide strands, can unexpectedly maintain or improve properties of the ds oligonucleotides described herein. For example, but not by way of limitation, the instant disclosure relates, in part, to ds oligonucleotides comprising a guide stranding comprising one or more of: (1) a backbone phosphoryl guanidine chiral center in the Sp configuration between the +3 nucleotide and the immediately downstream (+4) nucleotide; (2) a phosphorothioate chiral center in Rp or Sp configuration; (3) an Rp, Sp, or stereorandom non-negatively charged internucleotidic linkage where the 3′ nucleotide of a nucleotide pair linked by an Rp, Sp, or stereorandom non-negatively charged internucleotidic linkage comprises a 2′ modification, e.g., a 2′ F or a 2′-OMe; (4) a modified sugar selected from a 2′-F modification, 2′-H modification, 2′-OH modification, 2′-O-alkyl modification, e.g., 2′-O-methyl (OMe) modification, 2′-methoxyethyl (MOE) modification, 5′-alkyl modification, e.g., 5′-(R)-methyl or 5′-(S)-methyl, DNA, locked nucleic acid (LNA), unlocked nucleic acid (UNA), glycol nucleic acid (GNA), or Homo-DNA; and (5) a 5′ terminal modification selected from:
(a) 5′ PO modifications, such as, but not limited to:

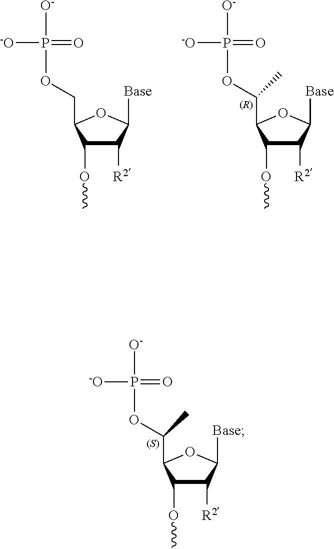

(b) 5′ VP modifications, such as, but not limited to:

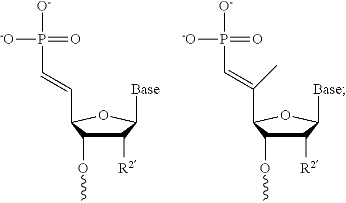

(c) 5′ MeP modifications, such as, but not limited to:

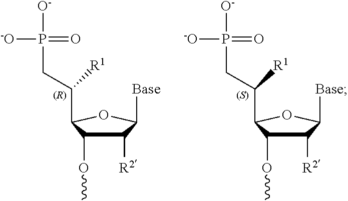

(d) 5′ PN and 5′ Triazole-P modifications, such as, but not limited to:

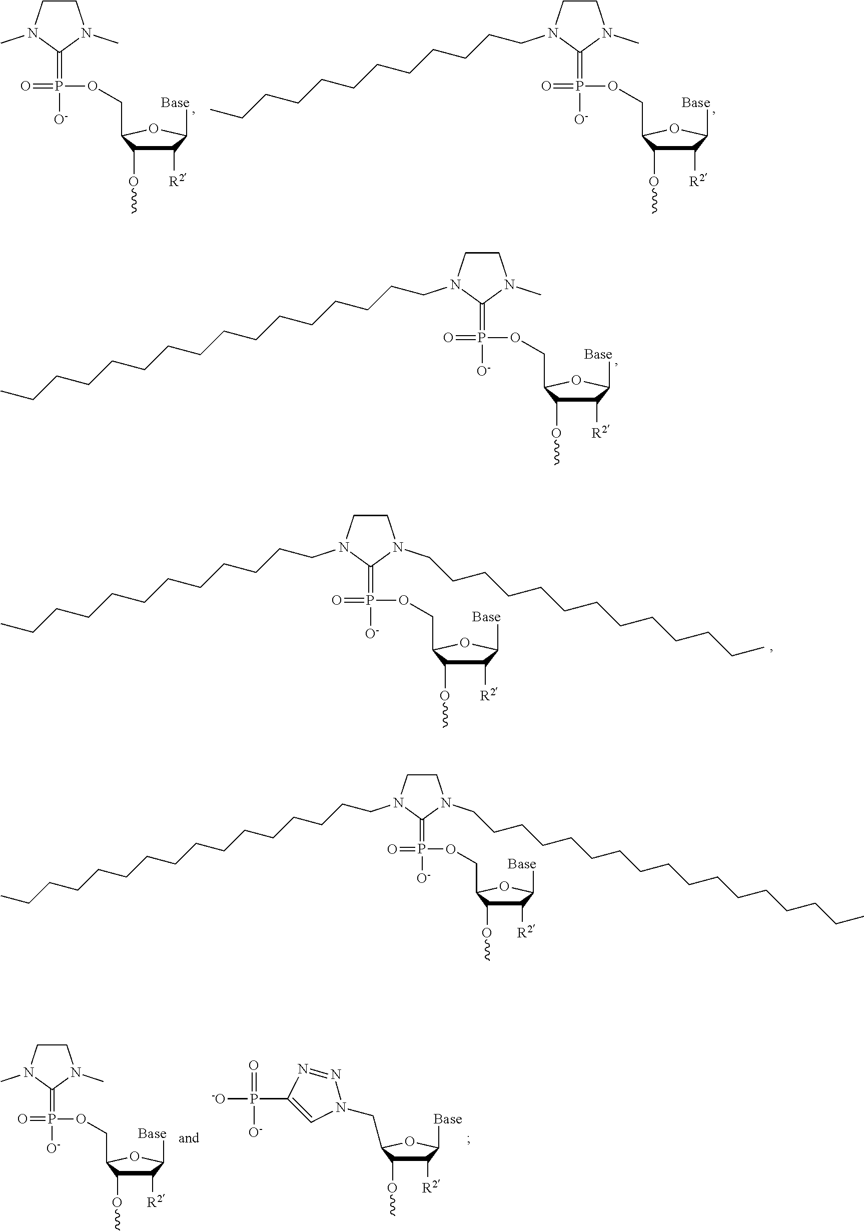

Wherein Base is selected from A, C, G, T, U, abasic and modified nucleobases;
R¹ is selected from an alkyl, methyl, ethyl, isopropyl, propyl, cyclohexyl, benzyl, phenyl, tolyl, xylyl, aryl, or arene group.
R^{2′} is selected from H, OH, O-alkyl, 0-Me, F, MOE, locked nucleic acid (LNA) bridges and bridged nucleic acid (BNA) bridges to the 4′ C, such as, but not limited to:

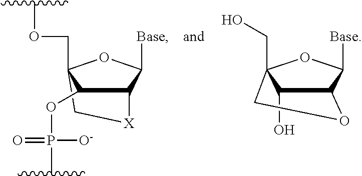

In certain embodiments, the one or more Rp, Sp, or stereorandom non-negatively charged internucleotidic linkage incorporated into the guide strand is an Rp non-negatively charged internucleotidic linkage. In certain embodiments, the one or more Rp, Sp, or stereorandom non-negatively charged internucleotidic linkage is an Sp non-negatively charged internucleotidic linkage. In certain embodiments, the one or more Rp, Sp, or stereorandom non-negatively charged internucleotidic linkage is a stereorandom non-negatively charged internucleotidic linkage.

In certain embodiments, the guide strand comprises, a backbone phosphorothioate chiral center in Sp configuration between the 5′ terminal (+1) nucleotide and the immediately downstream, i.e., in the 3′ direction, (+2) nucleotide, and a backbone phosphorothioate chiral center in the Rp configuration between the +2 nucleotide and the immediately downstream (+3) nucleotide. In certain embodiments, the 5′ terminal modification is

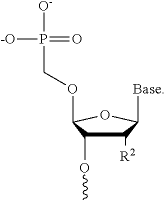

In some embodiments, the 5′ terminal modification is

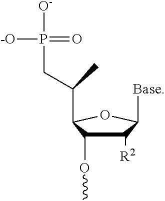

In certain embodiments, the 5′ terminal modification is

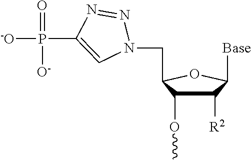

In certain embodiments, the guide strand comprises a 5′ terminal modification selected from, but not limited to, 5′ MeP modifications and 5′ Triazole-P modifications. In certain embodiments, the guide strand comprises the 5′ MeP modification

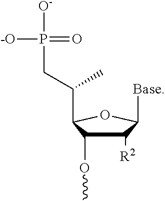

a backbone phosphorothioate chiral center in Sp configuration between the 5′ terminal (+1) nucleotide and the immediately downstream, i.e., in the 3′ direction, (+2) nucleotide, and a backbone phosphorothioate chiral center in the Rp configuration between the +2 nucleotide and the immediately downstream (+3) nucleotide.

In certain other embodiments, the present disclosure encompasses the recognition that stereochemistry, e.g., stereochemistry of chiral centers at the 5′ terminal nucleotide of guide strands, can unexpectedly maintain or improve properties of ds oligonucleotides wherein the guide strand of the ds oligonucleotide also comprises a phosphorothioate chiral center in Rp or Sp configuration. For example, but not by way of limitation, the instant disclosure relates, in part, to ds oligonucleotides comprising a guide stranding comprising: (1) a phosphorothioate chiral center in Rp or Sp configuration; (2) an Rp, Sp, or stereorandom non-negatively charged internucleotidic linkage where the 3′ nucleotide of a nucleotide pair linked by an Rp, Sp, or stereorandomnon-negatively charged internucleotidic linkage comprises a 2′ modification, e.g., a 2′ F or a 2′-OMe; and (3) a 5′ terminal modification selected from:
(a) 5′ PO nucleotides, such as, but not limited to:

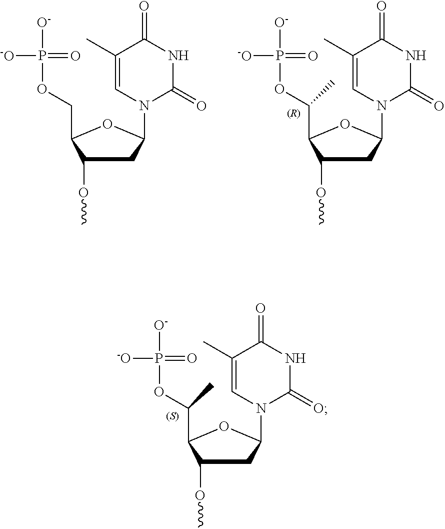

(b) 5′ VP nucleotides, such as, but not limited to:

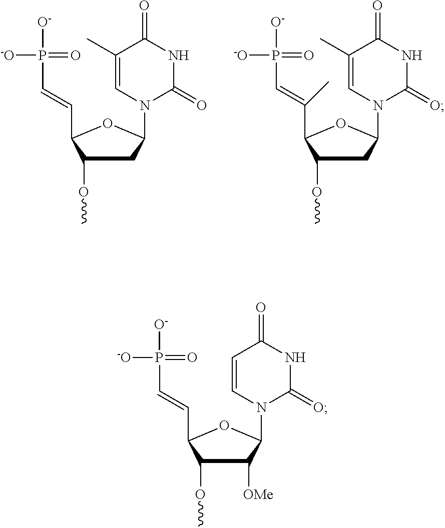

(c) 5′ MeP nucleotides, such as, but not limited to:

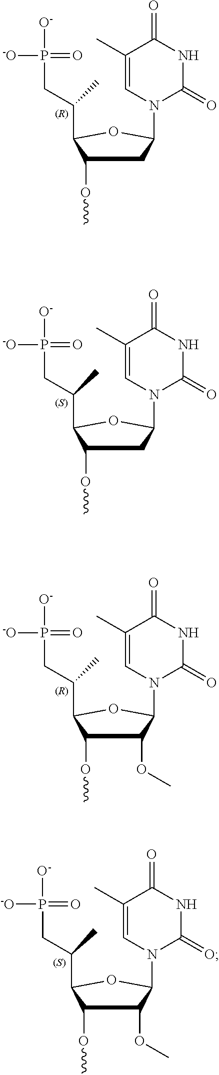

(d) 5′ PN and 5′ Triazole-P nucleotides, such as, but not limited to:

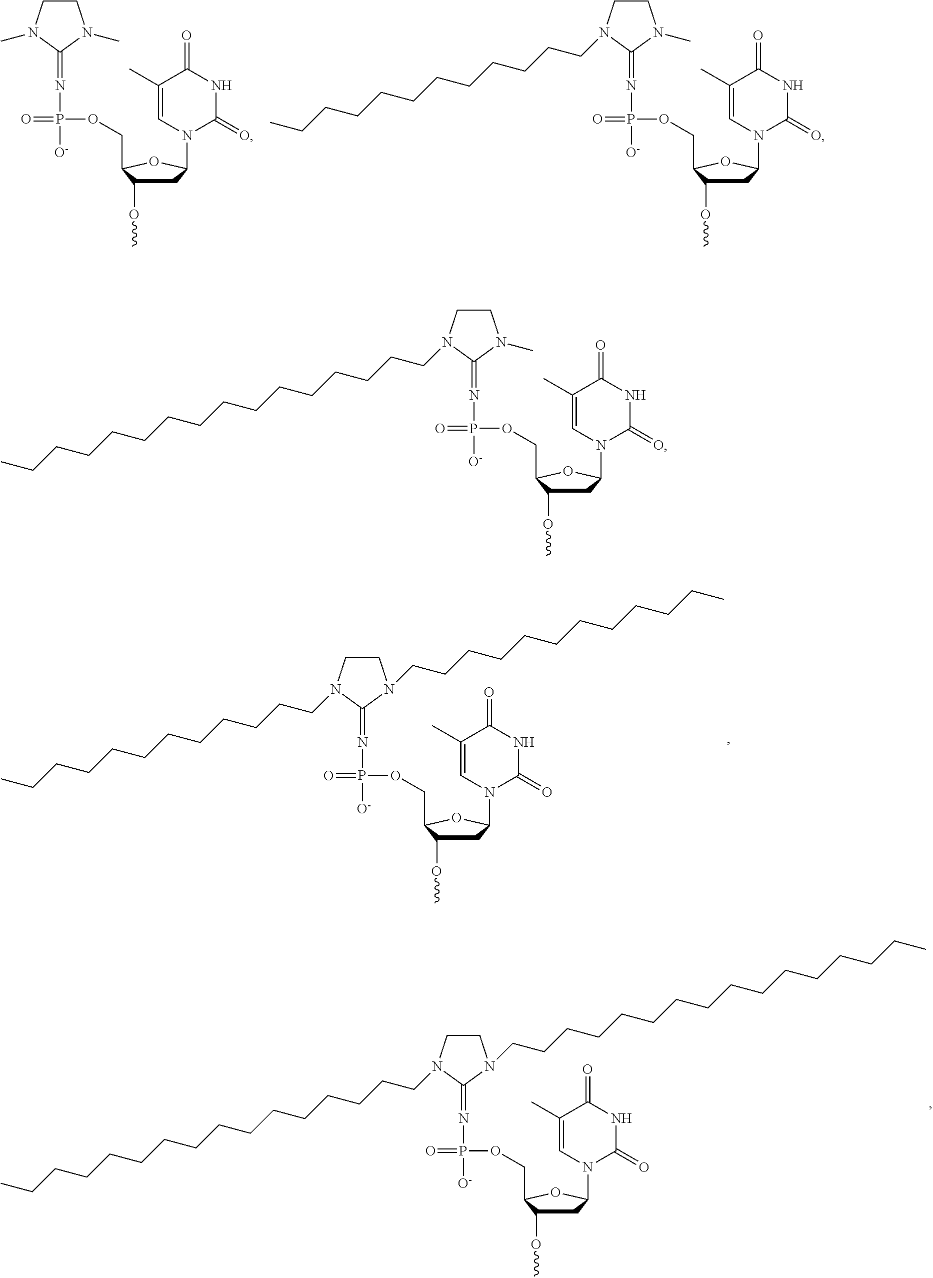

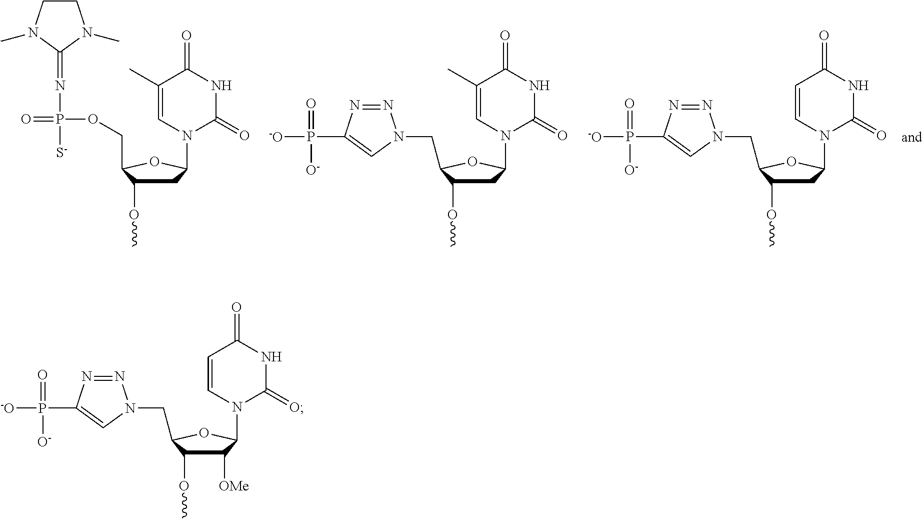

(e) 5′ abasic VP and 5′ abasic MeP nucleotides, such as, but not limited to.

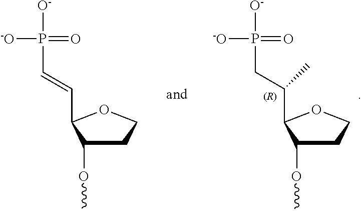

In certain embodiments, the one or more Rp, Sp, or stereorandom non-negatively charged internucleotidic linkage incorporated into the guide strand is an Rp non-negatively charged internucleotidic linkage. In certain embodiments, the one or more Rp, Sp, or stereorandom non-negatively charged internucleotidic linkage is an Sp non-negatively charged internucleotidic linkage. In certain embodiments, the one or more Rp, Sp, or stereorandom non-negatively charged internucleotidic linkage is a stereorandom non-negatively charged internucleotidic linkage.

In certain other embodiments, the present disclosure encompasses the recognition that stereochemistry, e.g., stereochemistry of chiral centers at the 5′ terminal nucleotide of guide strands, can unexpectedly maintain or improve properties of ds oligonucleotides wherein the guide strand of the ds oligonucleotide also comprises a phosphorothioate chiral center in Rp or Sp configuration. For example, but not by way of limitation, the instant disclosure relates, in part, to ds oligonucleotides comprising a guide stranding comprising: (1) a backbone phosphoryl guanidine chiral center in the Sp configuration between the +3 nucleotide and the immediately downstream (+4) nucleotide; (2) a backbone phosphoryl guanidine chiral center in the Rp configuration between the +7 nucleotide and the immediately downstream (+8) nucleotide; (3) a phosphorothioate chiral center in Rp or Sp configuration; and (4) a protected phosphonate selected from:

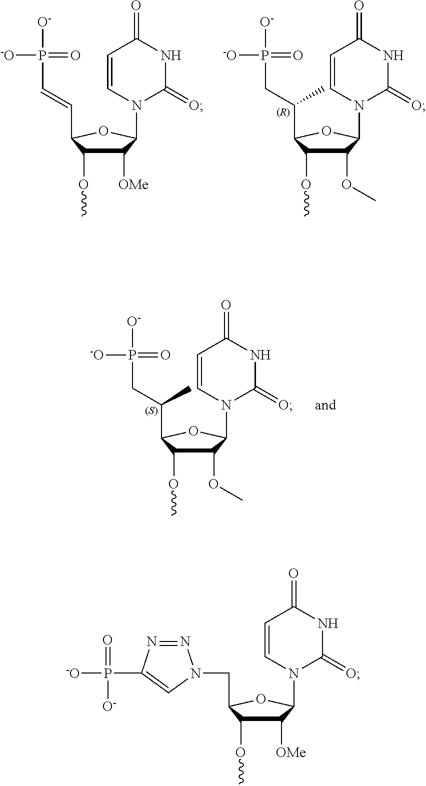

In certain embodiments, the one or more Rp, Sp, or stereorandom non-negatively charged internucleotidic linkage incorporated into the guide strand is an Rp non-negatively charged internucleotidic linkage. In certain embodiments, the one or more Rp, Sp, or stereorandom non-negatively charged internucleotidic linkage is an Sp non-negatively charged internucleotidic linkage. In certain embodiments, the one or more Rp, Sp, or stereorandom non-negatively charged internucleotidic linkage is a stereorandom non-negatively charged internucleotidic linkage.

In certain embodiments, the present disclosure encompasses the recognition that non-naturally occurring internucleotidic linkages, e.g., neutral internucleotidic linkages, can, in certain embodiments, be used to link one or more molecules to the double-stranded oligonucleotides described herein. In certain embodiments, such linked molecules can facilitate targeting and/or delivery of the double-stranded oligonucleotide. For example, but not limitation, such linked molecules an include lipophilic molecules. In certain embodiments, the linked molecule is a molecule comprising one or more GalNAc moieties. In certain embodiments, the linked molecule is a receptor. In certain embodiments, the linked molecule is a receptor ligand.

In certain embodiments, the present disclosure provides technologies for incorporating various additional chemical moieties into ds oligonucleotides. In certain embodiments, the present disclosure provides, for example, reagents and methods for introducing additional chemical moieties through nucleobases (e.g., by covalent linkage, optionally via a linker, to a site on a nucleobase).

In certain embodiments, the present disclosure provides technologies, e.g., ds oligonucleotide compositions and methods thereof, that achieve allele-specific suppression, wherein transcripts from one allele of a particular target gene is selectively knocked down relative to at least one other allele of the same gene.

Among other things, the present disclosure provides structural elements, technologies and/or features that can be incorporated into ds oligonucleotides and can impart or tune one or more properties thereof (e.g., relative to an otherwise identical ds oligonucleotide lacking the relevant technology or feature). In certain embodiments, the present disclosure documents that one or more provided technologies and/or features can usefully be incorporated into ds oligonucleotides of various sequences.

In certain embodiments, the present disclosure demonstrates that certain provided structural elements, technologies and/or features are particularly useful for ds oligonucleotides that participate in and/or direct RNAi mechanisms (e.g., RNAi agents). Regardless, however, the teachings of the present disclosure are not limited to ds oligonucleotides that participate in or operate via any particular mechanism. In certain embodiments, the present disclosure pertains to any ds oligonucleotide, useful for any purpose, which operates through any mechanism, and which comprises any sequence, structure or format (or portion thereof) described herein. In certain embodiments, the present disclosure provides a ds oligonucleotide, useful for any purpose, which operates through any mechanism, and which comprises any sequence, structure or format (or portion thereof) described herein, comprising a guide strand comprising backbone phosphoryl guanidine chiral centers in Sp configuration.

In certain embodiments, the guide strand comprises a backbone phosphoryl guanidine chiral center in the Sp configuration between the +3 nucleotide and the immediately downstream (+4) nucleotide, i.e., in the 3′ direction.

In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, further comprise a phosphoryl guanidine chiral center in the Sp configuration between the +7 and the immediately downstream (+8) nucleotide, between the +10 nucleotide and the immediately downstream (+11) nucleotide, between the +18 nucleotide and the immediately downstream (+19) nucleotide, or combinations thereof.

In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, further comprise one or a plurality of modified sugars. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the 5′ terminal (+1) nucleotide, second (+2) nucleotide, third (+3) nucleotide, fourth (+4) nucleotide, fifth (+5) nucleotide, seventh (+7) nucleotide, eighth (+8) nucleotide, ninth (+9) nucleotide, tenth (+10) nucleotide, twelfth (+12) nucleotide, sixteenth (+16) nucleotide, seventeenth (+17) nucleotide, eighteenth (+18) nucleotide, nineteenth (+19) nucleotide, twentieth (+20) nucleotide, twenty-first (+21) nucleotide, twenty-second (+22), and/or twenty-third (+23), the 3′ terminal, nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the 5′ terminal (+1) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified at the second (+2) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the third (+3) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the fourth (+4) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the fifth (+5) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar of the sixth (+6) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar of the seventh (+7) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar of the eighth (+8) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar of the ninth (+9) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar of the tenth (+10) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar of the twelfth (+12) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar of the sixteenth (+16) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar of the seventeenth (+17) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar of the eighteenth (+18) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar of the nineteenth (+19) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar of the twentieth (+20) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar of the twenty-first (+21) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar of the twenty-second (+22) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar of the twenty-third (+23), the 3′ terminal, nucleotide. In certain embodiments, the modified sugar comprises a 2′-F modification, 2′-H modification, 2′-OH modification, 2′-O-alkyl modification, e.g., 2′-O-methyl (OMe) modification, 2′-O—C16 lipid modification, 2′-methoxyethyl (MOE) modification, 5′-alkyl modification, e.g., 5′-(R)-methyl or 5′-(S)-methyl, DNA, locked nucleic acid (LNA), unlocked nucleic acid (UNA), glycol nucleic acid (GNA), or Homo-DNA.

In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the 5′ terminal (+1) nucleotide, second (+2) nucleotide, third (+3) nucleotide, fourth (+4) nucleotide, fifth (+5) nucleotide, and/or sixth (+6) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the 5′ terminal (+1) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the second (+2) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the third (+3) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the fourth (+4) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the fifth (+5) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the sixth (+6) nucleotide.

In certain embodiments, the present disclosure provides a ds oligonucleotide, useful for any purpose, which operates through any mechanism, and which comprises comprising a guide strand backbone phosphoryl guanidine chiral center in the Sp configuration between the +3 nucleotide and the immediately downstream (+4) nucleotide, i.e., in the 3′ direction, which further comprises:
(1) a guide strand comprising backbone phosphorothioate chiral centers in Sp configuration between the 3′ terminal nucleotide and the penultimate (N-1) nucleotide and as between the penultimate (N-1) nucleotide and the immediately upstream, i.e., in the 5′ direction, (N-2) nucleotide;
(2) a guide strand comprising backbone phosphorothioate chiral centers in Rp, Sp, or alternating configurations between the 5′ terminal (+1) nucleotide and the immediately downstream, i.e., in the 3′ direction, (+2) nucleotide and between the +2 nucleotide and the immediately downstream (+3) nucleotide;
(3) a guide strand comprising one or more backbone phosphorothioate chiral centers upstream, i.e., in the 5′ direction, relative to backbone phosphorothioate chiral centers in Sp configuration between the 3′ terminal nucleotide and the penultimate (N-1) nucleotide and as between the penultimate (N-1) nucleotide and the immediately upstream (N-2) nucleotide, where the upstream backbone phosphorothioate chiral centers are in Rp or Sp configuration;
(4) a guide strand comprising one or more backbone phosphorothioate chiral centers in Rp or Sp configuration between the 5′ terminal (+1) nucleotide and the immediately downstream (+2) nucleotide; and between the +2 nucleotide and the immediately downstream (+3) nucleotide;
(5) a guide strand comprising one or more backbone phosphorothioate chiral centers in Rp or Sp configuration where linkage occurs between any two adjacent nucleotides between the penultimate 3′ nucleotide of the guide strand, where N is the 3′ terminal nucleotide, and the upstream N-10 nucleotide;
(6) a guide strand comprising one or more backbone phosphoryl guanidine chiral centers in the Sp configuration between the +7 and the immediately downstream (+8), i.e., in the 3′ direction.
(7) a guide strand comprising a 5′ terminal modification;
(8) a passenger strand in combination with one or more of the aforementioned guide strands, comprising one or more backbone chiral centers in Rp or Sp configuration;
(9) a passenger strand in combination with one or more of the aforementioned guide strands, comprising one or more backbone phosphoryl guanidine chiral centers in the Rp configuration between the +7 nucleotide and the immediately downstream (+8) nucleotide, i.e., in the 3′ direction;
(10) a passenger strand in combination with one or more of the aforementioned guide strands, comprising one or more backbone phosphoryl guanidine chiral centers in the Rp configuration between the +15 nucleotide and the immediately downstream (+16) nucleotide, i.e., in the 3′ direction; and
(11) a passenger strand in combination with one or more of the aforementioned guide strands, comprising backbone phosphorothioate chiral centers in the Sp configuration between the 5′ terminal (+1) nucleotide and the immediately downstream, i.e., in the 3′ direction, (+2) nucleotide and between the 3′ terminal nucleotide and the penultimate (N-1) nucleotide;
   wherein the ds oligonucleotide further comprises one or more of:
(1) a guide strand where one or both of the 5′ and 3′ terminal dinucleotides are not linked by non-negatively charged internucleotidic linkages, i.e., the guide strand comprises one more Rp, Sp, or stereorandom non-negatively charged internucleotidic linkages downstream, i.e., in the 3′ direction, relative to the linkage between the 5′ terminal dinucleotide and/or upstream, i.e., in the 5′ direction, relative to the linkage between the 3′ terminal dinucleotide;
(2) a guide strand where one or more Rp, Sp, or stereorandom non-negatively charged internucleotidic linkage occurs between any two adjacent nucleotides between the second (+2) nucleotide relative to the 5′ terminal nucleotide of the guide strand and the penultimate 3′ (N-1) nucleotide of the guide strand, where N is the 3′ terminal nucleotide;
(3) a guide strand where an Rp, Sp, or stereorandom non-negatively charged internucleotidic linkage occurs between the tenth (+10) and eleventh (+11) nucleotides, relative to the 5′ terminal nucleotide;
(4) a guide strand where an Rp, Sp, or stereorandom non-negatively charged internucleotidic linkage occurs between the seventh (+7) and eighth (+8) nucleotides, relative to the 5′ terminal nucleotide;
(5) a guide strand comprising one or more backbone phosphorothioate chiral centers in Rp or Sp configuration where linkage occurs between any two adjacent nucleotides between the penultimate 3′ nucleotide of the guide strand, where N is the 3′ terminal nucleotide, and the upstream N-10 nucleotide;
(6) a passenger strand where one or more Rp, Sp, or stereorandom non-negatively charged internucleotidic linkage occurs upstream, i.e., in the 5′ direction, relative to the central nucleotide of the passenger strand;
(7) a passenger strand where one or more Rp, Sp, or stereorandom non-negatively charged internucleotidic linkage occurs downstream, i.e., in the 3′ direction, relative to the central nucleotide of the passenger strand;
(8) a passenger strand where one or more backbone phosphorothioate chiral centers in Rp or Sp configuration internucleotidic linkage occurs upstream, i.e., in the 5′ direction, relative to the central nucleotide of the passenger strand;
(9) a passenger strand where one or more backbone phosphorothioate chiral centers in Rp or Sp configuration internucleotidic linkage internucleotidic linkage occurs downstream, i.e., in the 3′ direction, relative to the central nucleotide of the passenger strand; and
(10) a passenger strand comprising one or more modified sugars, e.g. Homo-DNA, between the 5′ terminal (+1) nucleotide and the penultimate (N-1) nucleotide.

In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a Homo-DNA modification of the 5′ terminal (+1) nucleotide, sixth (+6) nucleotide, ninth (+9) nucleotide, tenth (+10) nucleotide, twelfth (+12) nucleotide, sixteenth (+16) nucleotide, seventeenth (+17) nucleotide, eighteenth (+18) nucleotide, nineteenth (+19) nucleotide, twentieth (+20) nucleotide, twenty-first (+21) nucleotide, twenty-second (+22), and/or twenty-third (+23), the 3′ terminal, nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a Homo-DNA modification of the 5′ terminal (+1) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a Homo-DNA modification of the sixth (+6) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a Homo-DNA modification of the ninth (+9) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a Homo-DNA modification of the tenth (+10) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a Homo-DNA modification of the twelfth (+12) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a Homo-DNA modification of the sixteenth (+16) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a Homo-DNA modification of the seventeenth (+17) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a Homo-DNA modification of the eighteenth (+18) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a Homo-DNA modification of the nineteenth (+19) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a Homo-DNA modification of the twentieth (+20) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a Homo-DNA modification of the twenty-first (+21) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a Homo-DNA modification of the twenty-second (+22) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a Homo-DNA modification of the twenty-third (+23), the 3′ terminal, nucleotide

In certain embodiments, the present disclosure provides a ds oligonucleotide, useful for any purpose, which operates through any mechanism, and which comprises comprising a guide strand backbone phosphoryl guanidine chiral center in the Sp configuration between the +3 nucleotide and the immediately downstream (+4) nucleotide, i.e., in the 3′ direction, which further comprises:
(1) a guide strand comprising backbone phosphorothioate chiral centers in Sp configuration between the 3′ terminal nucleotide and the penultimate (N-1) nucleotide and as between the penultimate (N-1) nucleotide and the immediately upstream, i.e., in the 5′ direction, (N-2) nucleotide;
(2) a guide strand comprising backbone phosphorothioate chiral centers in Rp, Sp, or alternating configurations between the 5′ terminal (+1) nucleotide and the immediately downstream, i.e., in the 3′ direction, (+2) nucleotide and between the +2 nucleotide and the immediately downstream (+3) nucleotide;
(3) a guide strand comprising one or more backbone phosphorothioate chiral centers upstream, i.e., in the 5′ direction, relative to backbone phosphorothioate chiral centers in Sp configuration between the 3′ terminal nucleotide and the penultimate (N-1) nucleotide and as between the penultimate (N-1) nucleotide and the immediately upstream (N-2) nucleotide, where the upstream backbone phosphorothioate chiral centers are in Rp or Sp configuration;
(4) a guide strand comprising one or more backbone phosphorothioate chiral centers in Rp or Sp configuration between the 5′ terminal (+1) nucleotide and the immediately downstream (+2) nucleotide; and between the +2 nucleotide and the immediately downstream (+3) nucleotide;
(5) a guide strand comprising one or more backbone phosphorothioate chiral centers in Rp or Sp configuration where linkage occurs between any two adjacent nucleotides between the penultimate 3′ nucleotide of the guide strand, where N is the 3′ terminal nucleotide, and the upstream N-10 nucleotide;
(6) a guide strand comprising one or more backbone phosphoryl guanidine chiral centers in the Sp configuration between the +7 and the immediately downstream (+8), i.e., in the 3′ direction.
(7) a guide strand comprising a 5′ terminal modification;
(8) a passenger strand in combination with one or more of the aforementioned guide strands, comprising one or more backbone chiral centers in Rp or Sp configuration;
(9) a passenger strand in combination with one or more of the aforementioned guide strands, comprising one or more backbone phosphoryl guanidine chiral centers in the Rp configuration between the +7 nucleotide and the immediately downstream (+8) nucleotide, i.e., in the 3′ direction;
(10) a passenger strand in combination with one or more of the aforementioned guide strands, comprising one or more backbone phosphoryl guanidine chiral centers in the Rp configuration between the +15 nucleotide and the immediately downstream (+16) nucleotide, i.e., in the 3′ direction; and
(11) a passenger strand in combination with one or more of the aforementioned guide strands, comprising backbone phosphorothioate chiral centers in the Sp configuration between the 5′ terminal (+1) nucleotide and the immediately downstream, i.e., in the 3′ direction, (+2) nucleotide and between the 3′ terminal nucleotide and the penultimate (N-1) nucleotide;
   wherein the ds oligonucleotide further comprises one or more of:
(1) a guide strand where one or both of the 5′ and 3′ terminal dinucleotides are not linked by non-negatively charged internucleotidic linkages, i.e., the guide strand comprises one more Rp, Sp, or stereorandom non-negatively charged internucleotidic linkages downstream, i.e., in the 3′ direction, relative to the linkage between the 5′ terminal dinucleotide and/or upstream, i.e., in the 5′ direction, relative to the linkage between the 3′ terminal dinucleotide;
(2) a guide strand where one or more Rp, Sp, or stereorandom non-negatively charged internucleotidic linkage occurs between any two adjacent nucleotides between the second (+2) nucleotide relative to the 5′ terminal nucleotide of the guide strand and the penultimate 3′ (N-1) nucleotide of the guide strand, where N is the 3′ terminal nucleotide;
(3) a guide strand where an Rp, Sp, or stereorandom non-negatively charged internucleotidic linkage occurs between the tenth (+10) and eleventh (+11) nucleotides, relative to the 5′ terminal nucleotide;
(4) a guide strand where an Rp, Sp, or stereorandom non-negatively charged internucleotidic linkage occurs between the seventh (+7) and eighth (+8) nucleotides, relative to the 5′ terminal nucleotide;
(5) a guide strand comprising one or more backbone phosphorothioate chiral centers in Rp or Sp configuration where linkage occurs between any two adjacent nucleotides between the penultimate 3′ nucleotide of the guide strand, where N is the 3′ terminal nucleotide, and the upstream N-10 nucleotide;
(6) a passenger strand where one or more Rp, Sp, or stereorandom non-negatively charged internucleotidic linkage occurs upstream, i.e., in the 5′ direction, relative to the central nucleotide of the passenger strand;
(7) a passenger strand where one or more Rp, Sp, or stereorandom non-negatively charged internucleotidic linkage occurs downstream, i.e., in the 3′ direction, relative to the central nucleotide of the passenger strand;
(8) a passenger strand where one or more backbone phosphorothioate chiral centers in Rp or Sp configuration internucleotidic linkage occurs upstream, i.e., in the 5′ direction, relative to the central nucleotide of the passenger strand;
(9) a passenger strand where one or more backbone phosphorothioate chiral centers in Rp or Sp configuration internucleotidic linkage internucleotidic linkage occurs downstream, i.e., in the 3′ direction, relative to the central nucleotide of the passenger strand; and
(10) a passenger strand comprising one or more modified sugars, e.g. Homo-DNA, between the 5′ terminal (+1) nucleotide and the penultimate (N-1) nucleotide.

In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a LNA modification of the 5′ terminal (+1) nucleotide, second (+2) nucleotide, third (+3) nucleotide, fourth (+4) nucleotide, fifth (+5) nucleotide, sixth (+6) nucleotide, seventh (+7) nucleotide, and/or eighth (+8) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a LNA modification of the 5′ terminal (+1) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a LNA modification of the second (+2) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a LNA modification of the third (+3) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a LNA modification of the fourth (+4) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a LNA modification of the fifth (+5) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a LNA modification of the sixth (+6) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a LNA modification of the seventh (+7) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a LNA modification of the eighth (+8) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-O-alkyl modification of the 5′ terminal (+1) nucleotide, second (+2) nucleotide, third (+3) nucleotide, fourth (+4) nucleotide, fifth (+5) nucleotide, sixth (+6) nucleotide, seventh (+7) nucleotide, and/or eighth (+8) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-O-alkyl modification of the 5′ terminal (+1) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-O-alkyl modification of the second (+2) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-O-alkyl modification of the third (+3) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-O-alkyl modification of the fourth (+4) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-O-alkyl modification of the fifth (+5) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-O-alkyl modification of the sixth (+6) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-O-alkyl modification of the seventh (+7) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-O-alkyl modification of the eighth (+8) nucleotide.

In certain embodiments, the present disclosure provides a ds oligonucleotide, useful for any purpose, which operates through any mechanism, and which comprises comprising a guide strand backbone phosphoryl guanidine chiral center in the Sp configuration between the +3 nucleotide and the immediately downstream (+4) nucleotide, i.e., in the 3′ direction, which further comprises:
(1) a guide strand comprising backbone phosphorothioate chiral centers in Sp configuration between the 3′ terminal nucleotide and the penultimate (N-1) nucleotide and as between the penultimate (N-1) nucleotide and the immediately upstream, i.e., in the 5′ direction, (N-2) nucleotide;
(2) a guide strand comprising backbone phosphorothioate chiral centers in Rp, Sp, or alternating configurations between the 5′ terminal (+1) nucleotide and the immediately downstream, i.e., in the 3′ direction, (+2) nucleotide and between the +2 nucleotide and the immediately downstream (+3) nucleotide;
(3) a guide strand comprising one or more backbone phosphorothioate chiral centers upstream, i.e., in the 5′ direction, relative to backbone phosphorothioate chiral centers in Sp configuration between the 3′ terminal nucleotide and the penultimate (N-1) nucleotide and as between the penultimate (N-1) nucleotide and the immediately upstream (N-2) nucleotide, where the upstream backbone phosphorothioate chiral centers are in Rp or Sp configuration;
(4) a guide strand comprising one or more backbone phosphorothioate chiral centers in Rp or Sp configuration between the 5′ terminal (+1) nucleotide and the immediately downstream (+2) nucleotide; and between the +2 nucleotide and the immediately downstream (+3) nucleotide;
(5) a guide strand comprising one or more backbone phosphorothioate chiral centers in Rp or Sp configuration where linkage occurs between any two adjacent nucleotides between the penultimate 3′ nucleotide of the guide strand, where N is the 3′ terminal nucleotide, and the upstream N-10 nucleotide;
(6) a guide strand comprising one or more backbone phosphoryl guanidine chiral centers in the Sp configuration between the +7 and the immediately downstream (+8), i.e., in the 3′ direction.
(7) a guide strand comprising a 5′ terminal modification;
(8) a passenger strand in combination with one or more of the aforementioned guide strands, comprising one or more backbone chiral centers in Rp or Sp configuration;
(9) a passenger strand in combination with one or more of the aforementioned guide strands, comprising one or more backbone phosphoryl guanidine chiral centers in the Rp configuration between the +7 nucleotide and the immediately downstream (+8) nucleotide, i.e., in the 3′ direction;
(10) a passenger strand in combination with one or more of the aforementioned guide strands, comprising one or more backbone phosphoryl guanidine chiral centers in the Rp configuration between the +15 nucleotide and the immediately downstream (+16) nucleotide, i.e., in the 3′ direction; and
(11) a passenger strand in combination with one or more of the aforementioned guide strands, comprising backbone phosphorothioate chiral centers in the Sp configuration between the 5′ terminal (+1) nucleotide and the immediately downstream, i.e., in the 3′ direction, (+2) nucleotide and between the 3′ terminal nucleotide and the penultimate (N-1) nucleotide;
   wherein the ds oligonucleotide further comprises one or more of:
(1) a guide strand where one or both of the 5′ and 3′ terminal dinucleotides are not linked by non-negatively charged internucleotidic linkages, i.e., the guide strand comprises one more Rp, Sp, or stereorandom non-negatively charged internucleotidic linkages downstream, i.e., in the 3′ direction, relative to the linkage between the 5′ terminal dinucleotide and/or upstream, i.e., in the 5′ direction, relative to the linkage between the 3′ terminal dinucleotide;
(2) a guide strand where one or more Rp, Sp, or stereorandom non-negatively charged internucleotidic linkage occurs between any two adjacent nucleotides between the second (+2) nucleotide relative to the 5′ terminal nucleotide of the guide strand and the penultimate 3′ (N-1) nucleotide of the guide strand, where N is the 3′ terminal nucleotide;
(3) a guide strand where an Rp, Sp, or stereorandom non-negatively charged internucleotidic linkage occurs between the tenth (+10) and eleventh (+11) nucleotides, relative to the 5′ terminal nucleotide;
(4) a guide strand where an Rp, Sp, or stereorandom non-negatively charged internucleotidic linkage occurs between the seventh (+7) and eighth (+8) nucleotides, relative to the 5′ terminal nucleotide;
(5) a guide strand comprising one or more backbone phosphorothioate chiral centers in Rp or Sp configuration where linkage occurs between any two adjacent nucleotides between the penultimate 3′ nucleotide of the guide strand, where N is the 3′ terminal nucleotide, and the upstream N-10 nucleotide;
(6) a passenger strand where one or more Rp, Sp, or stereorandom non-negatively charged internucleotidic linkage occurs upstream, i.e., in the 5′ direction, relative to the central nucleotide of the passenger strand;
(7) a passenger strand where one or more Rp, Sp, or stereorandom non-negatively charged internucleotidic linkage occurs downstream, i.e., in the 3′ direction, relative to the central nucleotide of the passenger strand;
(8) a passenger strand where one or more backbone phosphorothioate chiral centers in Rp or Sp configuration internucleotidic linkage occurs upstream, i.e., in the 5′ direction, relative to the central nucleotide of the passenger strand;
(9) a passenger strand where one or more backbone phosphorothioate chiral centers in Rp or Sp configuration internucleotidic linkage internucleotidic linkage occurs downstream, i.e., in the 3′ direction, relative to the central nucleotide of the passenger strand; and
(10) a passenger strand comprising one or more modified sugars, e.g. Homo-DNA, between the 5′ terminal (+1) nucleotide and the penultimate (N-1) nucleotide.

In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a DNA modification of the 5′ terminal (+1) nucleotide, second (+2) nucleotide, third (+3) nucleotide, fourth (+4) nucleotide, fifth (+5) nucleotide, sixth (+6) nucleotide, seventh (+7) nucleotide, and/or eighth (+8) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a DNA modification of the 5′ terminal (+1) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a DNA modification of the second (+2) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a DNA modification of the third (+3) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a DNA modification of the fourth (+4) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a DNA modification of the fifth (+5) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a DNA modification of the sixth (+6) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a DNA modification of the seventh (+7) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a DNA modification of the eighth (+8) nucleotide.

In certain embodiments, the present disclosure provides a ds oligonucleotide, useful for any purpose, which operates through any mechanism, and which comprises comprising a guide strand backbone phosphoryl guanidine chiral center in the Sp configuration between the +3 nucleotide and the immediately downstream (+4) nucleotide, i.e., in the 3′ direction, which further comprises:
(1) a guide strand comprising backbone phosphorothioate chiral centers in Sp configuration between the 3′ terminal nucleotide and the penultimate (N-1) nucleotide and as between the penultimate (N-1) nucleotide and the immediately upstream, i.e., in the 5′ direction, (N-2) nucleotide;
(2) a guide strand comprising backbone phosphorothioate chiral centers in Rp, Sp, or alternating configurations between the 5′ terminal (+1) nucleotide and the immediately downstream, i.e., in the 3′ direction, (+2) nucleotide and between the +2 nucleotide and the immediately downstream (+3) nucleotide;
(3) a guide strand comprising one or more backbone phosphorothioate chiral centers upstream, i.e., in the 5′ direction, relative to backbone phosphorothioate chiral centers in Sp configuration between the 3′ terminal nucleotide and the penultimate (N-1) nucleotide and as between the penultimate (N-1) nucleotide and the immediately upstream (N-2) nucleotide, where the upstream backbone phosphorothioate chiral centers are in Rp or Sp configuration;
(4) a guide strand comprising one or more backbone phosphorothioate chiral centers in Rp or Sp configuration between the 5′ terminal (+1) nucleotide and the immediately downstream (+2) nucleotide; and between the +2 nucleotide and the immediately downstream (+3) nucleotide;
(5) a guide strand comprising one or more backbone phosphorothioate chiral centers in Rp or Sp configuration where linkage occurs between any two adjacent nucleotides between the penultimate 3′ nucleotide of the guide strand, where N is the 3′ terminal nucleotide, and the upstream N-10 nucleotide;
(6) a guide strand comprising one or more backbone phosphoryl guanidine chiral centers in the Sp configuration between the +7 and the immediately downstream (+8), i.e., in the 3′ direction.
(7) a guide strand comprising a 5′ terminal modification;
(8) a passenger strand in combination with one or more of the aforementioned guide strands, comprising one or more backbone chiral centers in Rp or Sp configuration;
(9) a passenger strand in combination with one or more of the aforementioned guide strands, comprising one or more backbone phosphoryl guanidine chiral centers in the Rp configuration between the +7 nucleotide and the immediately downstream (+8) nucleotide, i.e., in the 3′ direction;
(10) a passenger strand in combination with one or more of the aforementioned guide strands, comprising one or more backbone phosphoryl guanidine chiral centers in the Rp configuration between the +15 nucleotide and the immediately downstream (+16) nucleotide, i.e., in the 3′ direction; and
(11) a passenger strand in combination with one or more of the aforementioned guide strands, comprising backbone phosphorothioate chiral centers in the Sp configuration between the 5′ terminal (+1) nucleotide and the immediately downstream, i.e., in the 3′ direction, (+2) nucleotide and between the 3′ terminal nucleotide and the penultimate (N-1) nucleotide;
   wherein the ds oligonucleotide further comprises one or more of.
(1) a guide strand where one or both of the 5′ and 3′ terminal dinucleotides are not linked by non-negatively charged internucleotidic linkages, i.e., the guide strand comprises one more Rp, Sp, or stereorandom non-negatively charged internucleotidic linkages downstream, i.e., in the 3′ direction, relative to the linkage between the 5′ terminal dinucleotide and/or upstream, i.e., in the 5′ direction, relative to the linkage between the 3′ terminal dinucleotide;
(2) a guide strand where one or more Rp, Sp, or stereorandom non-negatively charged internucleotidic linkage occurs between any two adjacent nucleotides between the second (+2) nucleotide relative to the 5′ terminal nucleotide of the guide strand and the penultimate 3′ (N-1) nucleotide of the guide strand, where N is the 3′ terminal nucleotide;
(3) a guide strand where an Rp, Sp, or stereorandom non-negatively charged internucleotidic linkage occurs between the tenth (+10) and eleventh (+11) nucleotides, relative to the 5′ terminal nucleotide;
(4) a guide strand where an Rp, Sp, or stereorandom non-negatively charged internucleotidic linkage occurs between the seventh (+7) and eighth (+8) nucleotides, relative to the 5′ terminal nucleotide;
(5) a guide strand comprising one or more backbone phosphorothioate chiral centers in Rp or Sp configuration where linkage occurs between any two adjacent nucleotides between the penultimate 3′ nucleotide of the guide strand, where N is the 3′ terminal nucleotide, and the upstream N-10 nucleotide;
(6) a passenger strand where one or more Rp, Sp, or stereorandom non-negatively charged internucleotidic linkage occurs upstream, i.e., in the 5′ direction, relative to the central nucleotide of the passenger strand;
(7) a passenger strand where one or more Rp, Sp, or stereorandom non-negatively charged internucleotidic linkage occurs downstream, i.e., in the 3′ direction, relative to the central nucleotide of the passenger strand;
(8) a passenger strand where one or more backbone phosphorothioate chiral centers in Rp or Sp configuration internucleotidic linkage occurs upstream, i.e., in the 5′ direction, relative to the central nucleotide of the passenger strand;
(9) a passenger strand where one or more backbone phosphorothioate chiral centers in Rp or Sp configuration internucleotidic linkage internucleotidic linkage occurs downstream, i.e., in the 3′ direction, relative to the central nucleotide of the passenger strand; and
(10) a passenger strand comprising one or more modified sugars, e.g. Homo-DNA, between the 5′ terminal (+1) nucleotide and the penultimate (N-1) nucleotide.

In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a GNA modification of the 5′ terminal (+1) nucleotide, second (+2) nucleotide, third (+3) nucleotide, fourth (+4) nucleotide, fifth (+5) nucleotide, sixth (+6) nucleotide, seventh (+7) nucleotide, and/or eighth (+8) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a GNA modification of the 5′ terminal (+1) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a GNA modification of the second (+2) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a GNA modification of the third (+3) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a GNA modification of the fourth (+4) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a GNA modification of the fifth (+5) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a GNA modification of the sixth (+6) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a GNA modification of the seventh (+7) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a GNA modification of the eighth (+8) nucleotide.

In certain embodiments, the present disclosure provides a ds oligonucleotide, useful for any purpose, which operates through any mechanism, and which comprises comprising a guide strand backbone phosphoryl guanidine chiral center in the Sp configuration between the +3 nucleotide and the immediately downstream (+4) nucleotide, i.e., in the 3′ direction, which further comprises:
(1) a guide strand comprising backbone phosphorothioate chiral centers in Sp configuration between the 3′ terminal nucleotide and the penultimate (N-1) nucleotide and as between the penultimate (N-1) nucleotide and the immediately upstream, i.e., in the 5′ direction, (N-2) nucleotide;
(2) a guide strand comprising backbone phosphorothioate chiral centers in Rp, Sp, or alternating configurations between the 5′ terminal (+1) nucleotide and the immediately downstream, i.e., in the 3′ direction, (+2) nucleotide and between the +2 nucleotide and the immediately downstream (+3) nucleotide;
(3) a guide strand comprising one or more backbone phosphorothioate chiral centers upstream, i.e., in the 5′ direction, relative to backbone phosphorothioate chiral centers in Sp configuration between the 3′ terminal nucleotide and the penultimate (N-1) nucleotide and as between the penultimate (N-1) nucleotide and the immediately upstream (N-2) nucleotide, where the upstream backbone phosphorothioate chiral centers are in Rp or Sp configuration;
(4) a guide strand comprising one or more backbone phosphorothioate chiral centers in Rp or Sp configuration between the 5′ terminal (+1) nucleotide and the immediately downstream (+2) nucleotide; and between the +2 nucleotide and the immediately downstream (+3) nucleotide;
(5) a guide strand comprising one or more backbone phosphorothioate chiral centers in Rp or Sp configuration where linkage occurs between any two adjacent nucleotides between the penultimate 3′ nucleotide of the guide strand, where N is the 3′ terminal nucleotide, and the upstream N-10 nucleotide;
(6) a guide strand comprising one or more backbone phosphoryl guanidine chiral centers in the Sp configuration between the +7 and the immediately downstream (+8), i.e., in the 3′ direction.
(7) a guide strand comprising a 5′ terminal modification;
(8) a passenger strand in combination with one or more of the aforementioned guide strands, comprising one or more backbone chiral centers in Rp or Sp configuration;
(9) a passenger strand in combination with one or more of the aforementioned guide strands, comprising one or more backbone phosphoryl guanidine chiral centers in the Rp configuration between the +7 nucleotide and the immediately downstream (+8) nucleotide, i.e., in the 3′ direction;
(10) a passenger strand in combination with one or more of the aforementioned guide strands, comprising one or more backbone phosphoryl guanidine chiral centers in the Rp configuration between the +15 nucleotide and the immediately downstream (+16) nucleotide, i.e., in the 3′ direction; and
(11) a passenger strand in combination with one or more of the aforementioned guide strands, comprising backbone phosphorothioate chiral centers in the Sp configuration between the 5′ terminal (+1) nucleotide and the immediately downstream, i.e., in the 3′ direction, (+2) nucleotide and between the 3′ terminal nucleotide and the penultimate (N-1) nucleotide;
   wherein the ds oligonucleotide further comprises one or more of.
(1) a guide strand where one or both of the 5′ and 3′ terminal dinucleotides are not linked by non-negatively charged internucleotidic linkages, i.e., the guide strand comprises one more Rp, Sp, or stereorandom non-negatively charged internucleotidic linkages downstream, i.e., in the 3′ direction, relative to the linkage between the 5′ terminal dinucleotide and/or upstream, i.e., in the 5′ direction, relative to the linkage between the 3′ terminal dinucleotide;
(2) a guide strand where one or more Rp, Sp, or stereorandom non-negatively charged internucleotidic linkage occurs between any two adjacent nucleotides between the second (+2) nucleotide relative to the 5′ terminal nucleotide of the guide strand and the penultimate 3′ (N-1) nucleotide of the guide strand, where N is the 3′ terminal nucleotide;
(3) a guide strand where an Rp, Sp, or stereorandom non-negatively charged internucleotidic linkage occurs between the tenth (+10) and eleventh (+11) nucleotides, relative to the 5′ terminal nucleotide;
(4) a guide strand where an Rp, Sp, or stereorandom non-negatively charged internucleotidic linkage occurs between the seventh (+7) and eighth (+8) nucleotides, relative to the 5′ terminal nucleotide;
(5) a guide strand comprising one or more backbone phosphorothioate chiral centers in Rp or Sp configuration where linkage occurs between any two adjacent nucleotides between the penultimate 3′ nucleotide of the guide strand, where N is the 3′ terminal nucleotide, and the upstream N-10 nucleotide;
(6) a passenger strand where one or more Rp, Sp, or stereorandom non-negatively charged internucleotidic linkage occurs upstream, i.e., in the 5′ direction, relative to the central nucleotide of the passenger strand;
(7) a passenger strand where one or more Rp, Sp, or stereorandom non-negatively charged internucleotidic linkage occurs downstream, i.e., in the 3′ direction, relative to the central nucleotide of the passenger strand;
(8) a passenger strand where one or more backbone phosphorothioate chiral centers in Rp or Sp configuration internucleotidic linkage occurs upstream, i.e., in the 5′ direction, relative to the central nucleotide of the passenger strand;
(9) a passenger strand where one or more backbone phosphorothioate chiral centers in Rp or Sp configuration internucleotidic linkage internucleotidic linkage occurs downstream, i.e., in the 3′ direction, relative to the central nucleotide of the passenger strand; and
(10) a passenger strand comprising one or more modified sugars, e.g. Homo-DNA, between the 5′ terminal (+1) nucleotide and the penultimate (N-1) nucleotide.

In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a (s)-GNA modification of the 5′ terminal (+1) nucleotide, second (+2) nucleotide, third (+3) nucleotide, fourth (+4) nucleotide, fifth (+5) nucleotide, sixth (+6) nucleotide, seventh (+7) nucleotide, and/or eighth (+8) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a (s)-GNA modification of the 5′ terminal (+1) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a (s)-GNA modification of the second (+2) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a (s)-GNA modification of the third (+3) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a (s)-GNA modification of the fourth (+4) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a (s)-GNA modification of the fifth (+5) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a (s)-GNA modification of the sixth (+6) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a (s)-GNA modification of the seventh (+7) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a (s)-GNA modification of the eighth (+8) nucleotide.

In certain embodiments, the present disclosure provides a ds oligonucleotide, useful for any purpose, which operates through any mechanism, and which comprises comprising a guide strand backbone phosphoryl guanidine chiral center in the Sp configuration between the +3 nucleotide and the immediately downstream (+4) nucleotide, i.e., in the 3′ direction, which further comprises:
(1) a guide strand comprising backbone phosphorothioate chiral centers in Sp configuration between the 3′ terminal nucleotide and the penultimate (N-1) nucleotide and as between the penultimate (N-1) nucleotide and the immediately upstream, i.e., in the 5′ direction, (N-2) nucleotide;
(2) a guide strand comprising backbone phosphorothioate chiral centers in Rp, Sp, or alternating configurations between the 5′ terminal (+1) nucleotide and the immediately downstream, i.e., in the 3′ direction, (+2) nucleotide and between the +2 nucleotide and the immediately downstream (+3) nucleotide;
(3) a guide strand comprising one or more backbone phosphorothioate chiral centers upstream, i.e., in the 5′ direction, relative to backbone phosphorothioate chiral centers in Sp configuration between the 3′ terminal nucleotide and the penultimate (N-1) nucleotide and as between the penultimate (N-1) nucleotide and the immediately upstream (N-2) nucleotide, where the upstream backbone phosphorothioate chiral centers are in Rp or Sp configuration;
(4) a guide strand comprising one or more backbone phosphorothioate chiral centers in Rp or Sp configuration between the 5′ terminal (+1) nucleotide and the immediately downstream (+2) nucleotide; and between the +2 nucleotide and the immediately downstream (+3) nucleotide;
(5) a guide strand comprising one or more backbone phosphorothioate chiral centers in Rp or Sp configuration where linkage occurs between any two adjacent nucleotides between the penultimate 3′ nucleotide of the guide strand, where N is the 3′ terminal nucleotide, and the upstream N-10 nucleotide;
(6) a guide strand comprising one or more backbone phosphoryl guanidine chiral centers in the Sp configuration between the +7 and the immediately downstream (+8), i.e., in the 3′ direction.
(7) a guide strand comprising a 5′ terminal modification;
(8) a passenger strand in combination with one or more of the aforementioned guide strands, comprising one or more backbone chiral centers in Rp or Sp configuration;
(9) a passenger strand in combination with one or more of the aforementioned guide strands, comprising one or more backbone phosphoryl guanidine chiral centers in the Rp configuration between the +7 nucleotide and the immediately downstream (+8) nucleotide, i.e., in the 3′ direction;
(10) a passenger strand in combination with one or more of the aforementioned guide strands, comprising one or more backbone phosphoryl guanidine chiral centers in the Rp configuration between the +15 nucleotide and the immediately downstream (+16) nucleotide, i.e., in the 3′ direction; and
(11) a passenger strand in combination with one or more of the aforementioned guide strands, comprising backbone phosphorothioate chiral centers in the Sp configuration between the 5′ terminal (+1) nucleotide and the immediately downstream, i.e., in the 3′ direction, (+2) nucleotide and between the 3′ terminal nucleotide and the penultimate (N-1) nucleotide;
   wherein the ds oligonucleotide further comprises one or more of:
(1) a guide strand where one or both of the 5′ and 3′ terminal dinucleotides are not linked by non-negatively charged internucleotidic linkages, i.e., the guide strand comprises one more Rp, Sp, or stereorandom non-negatively charged internucleotidic linkages downstream, i.e., in the 3′ direction, relative to the linkage between the 5′ terminal dinucleotide and/or upstream, i.e., in the 5′ direction, relative to the linkage between the 3′ terminal dinucleotide;
(2) a guide strand where one or more Rp, Sp, or stereorandom non-negatively charged internucleotidic linkage occurs between any two adjacent nucleotides between the second (+2) nucleotide relative to the 5′ terminal nucleotide of the guide strand and the penultimate 3′ (N-1) nucleotide of the guide strand, where N is the 3′ terminal nucleotide;
(3) a guide strand where an Rp, Sp, or stereorandom non-negatively charged internucleotidic linkage occurs between the tenth (+10) and eleventh (+11) nucleotides, relative to the 5′ terminal nucleotide;
(4) a guide strand where an Rp, Sp, or stereorandom non-negatively charged internucleotidic linkage occurs between the seventh (+7) and eighth (+8) nucleotides, relative to the 5′ terminal nucleotide;
(5) a guide strand comprising one or more backbone phosphorothioate chiral centers in Rp or Sp configuration where linkage occurs between any two adjacent nucleotides between the penultimate 3′ nucleotide of the guide strand, where N is the 3′ terminal nucleotide, and the upstream N-10 nucleotide;
(6) a passenger strand where one or more Rp, Sp, or stereorandom non-negatively charged internucleotidic linkage occurs upstream, i.e., in the 5′ direction, relative to the central nucleotide of the passenger strand;
(7) a passenger strand where one or more Rp, Sp, or stereorandom non-negatively charged internucleotidic linkage occurs downstream, i.e., in the 3′ direction, relative to the central nucleotide of the passenger strand;
(8) a passenger strand where one or more backbone phosphorothioate chiral centers in Rp or Sp configuration internucleotidic linkage occurs upstream, i.e., in the 5′ direction, relative to the central nucleotide of the passenger strand;
(9) a passenger strand where one or more backbone phosphorothioate chiral centers in Rp or Sp configuration internucleotidic linkage internucleotidic linkage occurs downstream, i.e., in the 3′ direction, relative to the central nucleotide of the passenger strand; and
(10) a passenger strand comprising one or more modified sugars, e.g. Homo-DNA, between the 5′ terminal (+1) nucleotide and the penultimate (N-1) nucleotide.

In certain embodiments, the ds oligonucleotide further comprises a 2′ modification, e.g., a 2′ F modification, of the 3′ nucleotide of a nucleotide pair linked by an Rp, Sp, or stereorandom non-negatively charged internucleotidic linkage. In certain embodiments, the one or more Rp, Sp, or stereorandom non-negatively charged internucleotidic linkage incorporated into the guide or passenger strand is an Rp non-negatively charged internucleotidic linkage. In certain embodiments, the one or more Rp, Sp, or stereorandom non-negatively charged internucleotidic linkage is an Sp non-negatively charged internucleotidic linkage. In certain embodiments, the one or more Rp, Sp, or stereorandom non-negatively charged internucleotidic linkage is a stereorandom non-negatively charged internucleotidic linkage. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, further comprise one or a plurality of modified sugars. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the 5′ terminal (+1) nucleotide, second (+2) nucleotide, third (+3) nucleotide, fourth (+4) nucleotide, fifth (+5) nucleotide, sixth (+6) nucleotide, seventh (+7) nucleotide, eighth (+8) nucleotide, ninth (+9) nucleotide, tenth (+10) nucleotide, twelfth (+12) nucleotide, sixteenth (+16) nucleotide, seventeenth (+17) nucleotide, eighteenth (+18) nucleotide, nineteenth (+19) nucleotide, twentieth (+20) nucleotide, twenty-first (+21) nucleotide, twenty-second (+22), and/or twenty-third (+23), the 3′ terminal, nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the 5′ terminal (+1) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the second (+2) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the third (+3) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the fourth (+4) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the fifth (+5) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the sixth (+6) nucleotide.

In certain embodiments, the modified sugar comprises a 2′-F modification, 2′-H modification, 2′-OH modification, 2′-O-alkyl modification, e.g., 2′-O-methyl (OMe) modification, 2′-methoxyethyl (MOE) modification, 5′-alkyl modification, e.g., 5′-(R)-methyl or 5′-(S)-methyl, DNA, locked nucleic acid (LNA), unlocked nucleic acid (UNA), glycol nucleic acid (GNA), or Homo-DNA.

In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the 5′ terminal (+1) nucleotide, second (+2) nucleotide, third (+3) nucleotide, fourth (+4) nucleotide, fifth (+5) nucleotide, and/or sixth (+6) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the 5′ terminal (+1) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the second (+2) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the third (+3) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the fourth (+4) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the fifth (+5) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the sixth (+6) nucleotide.

In certain embodiments, the provided ds oligonucleotides may participate in (e.g., direct) RNAi mechanisms. In certain embodiments, provided ds oligonucleotides may participate in RNase H (ribonuclease H) mechanisms. In certain embodiments, provided ds oligonucleotides may act as translational inhibitors (e.g., may provide steric blocks of translation).

In certain embodiments, the ds oligonucleotide comprises a guide strand backbone phosphoryl guanidine chiral center in the Sp configuration between the +3 nucleotide and the immediately downstream (+4) nucleotide, i.e., in the 3′ direction, and the guide strand further comprises backbone phosphorothioate chiral centers in Sp configuration between the 3′ terminal nucleotide and the penultimate (N-1) nucleotide and as between the penultimate (N-1) nucleotide and the immediately upstream (N-2) nucleotide, and one or more of:
(1) a guide strand where one or both of the 5′ and 3′ terminal dinucleotides are not linked by non-negatively charged internucleotidic linkages, i.e., the guide strand comprises one more non-negatively charged internucleotidic linkages downstream, i.e., in the 3′ direction, relative to the linkage between the 5′ terminal dinucleotide and/or upstream, i.e., in the 5′ direction, relative to the linkage between the 3′ terminal dinucleotide;
(2) a guide strand where one or more Rp, Sp, or stereorandom non-negatively charged internucleotidic linkage occurs between any two adjacent nucleotides between the second (+2) nucleotide relative to the 5′ terminal nucleotide of the guide strand and the penultimate 3′ (N-1) nucleotide of the guide strand, where N is the 3′ terminal nucleotide;
(4) a guide strand where an Rp, Sp, or stereorandom non-negatively charged internucleotidic linkage occurs between the seventh (+7) and eighth (+8) nucleotides, relative to the 5′ terminal nucleotide;
(5) a guide strand comprising one or more backbone phosphorothioate chiral centers in Rp or Sp configuration where linkage occurs between any two adjacent nucleotides between the penultimate 3′ nucleotide of the guide strand, where N is the 3′ terminal nucleotide, and the upstream N-10 nucleotide;
(6) a passenger strand where one or more Rp, Sp, or stereorandom non-negatively charged internucleotidic linkage occurs upstream, i.e., in the 5′ direction, relative to the central nucleotide of the passenger strand;
(7) a passenger strand where one or more Rp, Sp, or stereorandom non-negatively charged internucleotidic linkage occurs downstream, i.e., in the 3′ direction, relative to the central nucleotide of the passenger strand;
(8) a passenger strand where one or more backbone phosphorothioate chiral centers in Rp or Sp configuration internucleotidic linkage occurs upstream, i.e., in the 5′ direction, relative to the central nucleotide of the passenger strand;
(9) a passenger strand where one or more backbone phosphorothioate chiral centers in Rp or Sp configuration internucleotidic linkage internucleotidic linkage occurs downstream, i.e., in the 3′ direction, relative to the central nucleotide of the passenger strand; and
(10) a passenger strand comprising one or more modified sugars, e.g. Homo-DNA, between the 5′ terminal (+1) nucleotide and the penultimate (N-1) nucleotide.
   In certain embodiments, the ds oligonucleotide further comprises a 2′ modification, e.g., a 2′ F modification, of the 3′ nucleotide of a nucleotide pair linked by an Rp, Sp, or stereorandom non-negatively charged internucleotidic linkage, and the passenger strand comprises 0-n Rp, Sp, or stereorandom non-negatively charged internucleotidic linkages, where n is about 1 to 49.

In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, further comprise one or a plurality of modified sugars. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the 5′ terminal (+1) nucleotide, second (+2) nucleotide, third (+3) nucleotide, fourth (+4) nucleotide, fifth (+5) nucleotide, sixth (+6) nucleotide, seventh (+7) nucleotide, eighth (+8) nucleotide, ninth (+9) nucleotide, tenth (+10) nucleotide, twelfth (+12) nucleotide, sixteenth (+16) nucleotide, seventeenth (+17) nucleotide, eighteenth (+18) nucleotide, nineteenth (+19) nucleotide, twentieth (+20) nucleotide, twenty-first (+21) nucleotide, twenty-second (+22), and/or twenty-third (+23), the 3′ terminal, nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the 5′ terminal (+1) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the second (+2) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the third (+3) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the fourth (+4) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the fifth (+5) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the sixth (+6) nucleotide.

In certain embodiments, the modified sugar comprises a 2′-F modification, 2′-H modification, 2′-OH modification, 2′-O-alkyl modification, e.g., 2′-O-methyl (OMe) modification, 2′-methoxyethyl (MOE) modification, 5′-alkyl modification, e.g., 5′-(R)-methyl or 5′-(S)-methyl, DNA, locked nucleic acid (LNA), unlocked nucleic acid (UNA), glycol nucleic acid (GNA), or Homo-DNA. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the 5′ terminal (+1) nucleotide, second (+2) nucleotide, third (+3) nucleotide, fourth (+4) nucleotide, fifth (+5) nucleotide, and/or sixth (+6) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the 5′ terminal (+1) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the second (+2) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the third (+3) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the fourth (+4) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the fifth (+5) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the sixth (+6) nucleotide.

In certain embodiments, the passenger strand comprises an Sp backbone phosphorothioate chiral center between the 5′ terminal (+1) nucleotide and the immediately downstream (+2) nucleotide and an Sp backbone phosphorothioate chiral center between the penultimate (N-1) nucleotide and the 3′ terminal (N) nucleotide.

In certain embodiments, the ds oligonucleotide comprises a guide strand backbone phosphoryl guanidine chiral center in the Sp configuration between the +3 nucleotide and the immediately downstream (+4) nucleotide, i.e., in the 3′ direction, and the guide strand further comprises backbone phosphorothioate chiral centers in Rp, Sp, or alternating configurations between the 5′ terminal (+1) nucleotide and the immediately downstream (+2) nucleotide and between the +2 nucleotide and the immediately downstream (+3) nucleotide, and one or more of
(1) a guide strand where one or both of the 5′ and 3′ terminal dinucleotides are not linked by non-negatively charged internucleotidic linkages, i.e., the guide strand comprises one more non-negatively charged internucleotidic linkages downstream, i.e., in the 3′ direction, relative to the linkage between the 5′ terminal dinucleotide and/or upstream, i.e., in the 5′ direction, relative to the linkage between the 3′ terminal dinucleotide;
(2) a guide strand where one or more Rp, Sp, or stereorandom non-negatively charged internucleotidic linkage occurs between any two adjacent nucleotides between the second (+2) nucleotide relative to the 5′ terminal nucleotide of the guide strand and the penultimate 3′ (N-1) nucleotide of the guide strand, where N is the 3′ terminal nucleotide;
(3) a guide strand where an Rp, Sp, or stereorandom non-negatively charged internucleotidic linkage occurs between the tenth (+10) and eleventh (+11) nucleotides, relative to the 5′ terminal nucleotide; (4) a guide strand where an Rp, Sp, or stereorandom non-negatively charged internucleotidic linkage occurs between the seventh (+7) and eighth (+8) nucleotides, relative to the 5′ terminal nucleotide;
(5) a guide strand comprising one or more backbone phosphorothioate chiral centers in Rp or Sp configuration where linkage occurs between any two adjacent nucleotides between the penultimate 3′ nucleotide of the guide strand, where N is the 3′ terminal nucleotide, and the upstream N-10 nucleotide;
(6) a passenger strand where one or more Rp, Sp, or stereorandom non-negatively charged internucleotidic linkage occurs upstream, i.e., in the 5′ direction, relative to the central nucleotide of the passenger strand;
(7) a passenger strand where one or more Rp, Sp, or stereorandom non-negatively charged internucleotidic linkage occurs downstream, i.e., in the 3′ direction, relative to the central nucleotide of the passenger strand;
(8) a passenger strand where one or more backbone phosphorothioate chiral centers in Rp or Sp configuration internucleotidic linkage occurs upstream, i.e., in the 5′ direction, relative to the central nucleotide of the passenger strand;
(9) a passenger strand where one or more backbone phosphorothioate chiral centers in Rp or Sp configuration internucleotidic linkage internucleotidic linkage occurs downstream, i.e., in the 3′ direction, relative to the central nucleotide of the passenger strand; and
(10) a passenger strand comprising one or more modified sugars, e.g. Homo-DNA, between the 5′ terminal (+1) nucleotide and the penultimate (N-1) nucleotide.
   In certain embodiments, the ds oligonucleotide further comprises a 2′ modification, e.g., a 2′ F modification, of the 3′ nucleotide of a nucleotide pair linked by an Rp, Sp, or stereorandom non-negatively charged internucleotidic linkage, and the passenger strand comprises 0-n Rp, Sp, or stereorandom non-negatively charged internucleotidic linkages, where n is about 1 to 49.

In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, further comprise a modified sugar. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the 5′ terminal (+1) nucleotide, second (+2) nucleotide, third (+3) nucleotide, fourth (+4) nucleotide, fifth (+5) nucleotide, sixth (+6) nucleotide, seventh (+7) nucleotide, eighth (+8) nucleotide, ninth (+9) nucleotide, tenth (+10) nucleotide, twelfth (+12) nucleotide, sixteenth (+16) nucleotide, seventeenth (+17) nucleotide, eighteenth (+18) nucleotide, nineteenth (+19) nucleotide, twentieth (+20) nucleotide, twenty-first (+21) nucleotide, twenty-second (+22), and/or twenty-third (+23), the 3′ terminal, nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the 5′ terminal (+1) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the second (+2) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the third (+3) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the fourth (+4) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the fifth (+5) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the sixth (+6) nucleotide.

In certain embodiments, the modified sugar comprises a 2′-F modification, 2′-H modification, 2′-OH modification, 2′-O-alkyl modification, e.g., 2′-O-methyl (OMe) modification, 2′-methoxyethyl (MOE) modification, 5′-alkyl modification, e.g., 5′-(R)-methyl or 5′-(S)-methyl, DNA, locked nucleic acid (LNA), unlocked nucleic acid (UNA), glycol nucleic acid (GNA), or Homo-DNA.

In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the 5′ terminal (+1) nucleotide, second (+2) nucleotide, third (+3) nucleotide, fourth (+4) nucleotide, fifth (+5) nucleotide, and/or sixth (+6) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the 5′ terminal (+1) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the second (+2) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the third (+3) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the fourth (+4) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the fifth (+5) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the sixth (+6) nucleotide.

In certain embodiments, the one or more Rp, Sp, or stereorandom non-negatively charged internucleotidic linkage incorporated into the guide or passenger strand is an Rp non-negatively charged internucleotidic linkage. In certain embodiments, the one or more Rp, Sp, or stereorandom non-negatively charged internucleotidic linkage is an Sp non-negatively charged internucleotidic linkage. In certain embodiments, the one or more Rp, Sp, or stereorandom non-negatively charged internucleotidic linkage is a stereorandom non-negatively charged internucleotidic linkage. In certain embodiments, the passenger strand comprises an Sp backbone phosphorothioate chiral center between the 5′ terminal (+1) nucleotide and the immediately downstream (+2) nucleotide and an Sp backbone phosphorothioate chiral center between the penultimate (N-1) nucleotide and the 3′ terminal (N) nucleotide.

In certain embodiments, the ds oligonucleotide comprises a guide strand backbone phosphoryl guanidine chiral center in the Sp configuration between the +3 nucleotide and the immediately downstream (+4) nucleotide, i.e., in the 3′ direction, and the guide strand further comprises one or more backbone phosphorothioate chiral centers in Rp or Sp configuration upstream of backbone phosphorothioate chiral centers in Sp configuration between the 3′ terminal nucleotide and the penultimate (N-1) nucleotide and as between the penultimate (N-1) nucleotide and the immediately upstream (N-2) nucleotide, and one or more of
(1) a guide strand where one or both of the 5′ and 3′ terminal dinucleotides are not linked by non-negatively charged internucleotidic linkages, i.e., the guide strand comprises one more non-negatively charged internucleotidic linkages downstream, i.e., in the 3′ direction, relative to the linkage between the 5′ terminal dinucleotide and/or upstream, i.e., in the 5′ direction, relative to the linkage between the 3′ terminal dinucleotide;
(2) a guide strand where one or more Rp, Sp, or stereorandom non-negatively charged internucleotidic linkage occurs between any two adjacent nucleotides between the second (+2) nucleotide relative to the 5′ terminal nucleotide of the guide strand and the penultimate 3′ (N-1) nucleotide of the guide strand, where N is the 3′ terminal nucleotide;
(3) a guide strand where an Rp, Sp, or stereorandom non-negatively charged internucleotidic linkage occurs between the tenth (+10) and eleventh (+11) nucleotides, relative to the 5′ terminal nucleotide;
(4) a guide strand where an Rp, Sp, or stereorandom non-negatively charged internucleotidic linkage occurs between the seventh (+7) and eighth (+8) nucleotides, relative to the 5′ terminal nucleotide;
(5) a guide strand comprising one or more backbone phosphorothioate chiral centers in Rp or Sp configuration where linkage occurs between any two adjacent nucleotides between the penultimate 3′ nucleotide of the guide strand, where N is the 3′ terminal nucleotide, and the upstream N-10 nucleotide;
(6) a passenger strand where one or more Rp, Sp, or stereorandom non-negatively charged internucleotidic linkage occurs upstream, i.e., in the 5′ direction, relative to the central nucleotide of the passenger strand;
(7) a passenger strand where one or more Rp, Sp, or stereorandom non-negatively charged internucleotidic linkage occurs downstream, i.e., in the 3′ direction, relative to the central nucleotide of the passenger strand;
(8) a passenger strand where one or more backbone phosphorothioate chiral centers in Rp or Sp configuration internucleotidic linkage occurs upstream, i.e., in the 5′ direction, relative to the central nucleotide of the passenger strand;
(9) a passenger strand where one or more backbone phosphorothioate chiral centers in Rp or Sp configuration internucleotidic linkage internucleotidic linkage occurs downstream, i.e., in the 3′ direction, relative to the central nucleotide of the passenger strand; and
(10) a passenger strand comprising one or more modified sugars, e.g. Homo-DNA, between the 5′ terminal (+1) nucleotide and the penultimate (N-1) nucleotide.
   In certain embodiments, the ds oligonucleotide further comprises a 2′ modification, e.g., a 2′ F modification, of the 3′ nucleotide of a nucleotide pair linked by an Rp, Sp, or stereorandom non-negatively charged internucleotidic linkage, and the passenger strand comprises 0-n Rp, Sp, or stereorandom non-negatively charged internucleotidic linkages, where n is about 1 to 49.

In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, further comprise one or a plurality of modified sugars. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the 5′ terminal (+1) nucleotide, second (+2) nucleotide, third (+3) nucleotide, fourth (+4) nucleotide, fifth (+5) nucleotide, sixth (+6) nucleotide, seventh (+7)nucleotide, eighth (+8) nucleotide, ninth (+9) nucleotide, tenth (+10) nucleotide, twelfth (+12) nucleotide, sixteenth (+16) nucleotide, seventeenth (+17) nucleotide, eighteenth (+18) nucleotide, nineteenth (+19) nucleotide, twentieth (+20) nucleotide, twenty-first (+21) nucleotide, twenty-second (+22), and/or twenty-third (+23), the 3′ terminal, nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the 5′ terminal (+1) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the second (+2) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the third (+3) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the fourth (+4) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the fifth (+5) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the sixth (+6) nucleotide.

In certain embodiments, the modified sugar comprises a 2′-F modification, 2′-H modification, 2′-OH modification, 2′-O-alkyl modification, e.g., 2′-O-methyl (OMe) modification, 2′-methoxyethyl (MOE) modification, 5′-alkyl modification, e.g., 5′-(R)-methyl or 5′-(S)-methyl, DNA, locked nucleic acid (LNA), unlocked nucleic acid (UNA), glycol nucleic acid (GNA), or Homo-DNA. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the 5′ terminal (+1) nucleotide, second (+2) nucleotide, third (+3) nucleotide, fourth (+4) nucleotide, fifth (+5) nucleotide, and/or sixth (+6) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the 5′ terminal (+1) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the second (+2) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the third (+3) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the fourth (+4) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the fifth (+5) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the sixth (+6) nucleotide.

In certain embodiments, the one or more Rp, Sp, or stereorandom non-negatively charged internucleotidic linkage incorporated into the guide or passenger strand is an Rp non-negatively charged internucleotidic linkage. In certain embodiments, the one or more Rp, Sp, or stereorandom non-negatively charged internucleotidic linkage is an Sp non-negatively charged internucleotidic linkage. In certain embodiments, the one or more Rp, Sp, or stereorandom non-negatively charged internucleotidic linkage is a stereorandom non-negatively charged internucleotidic linkage. In certain further embodiments, the passenger strand comprises an Sp backbone phosphorothioate chiral center between the 5′ terminal (+1) nucleotide and the immediately downstream (+2) nucleotide and an Sp backbone phosphorothioate chiral center between the penultimate (N-1) nucleotide and the 3′ terminal (N) nucleotide.

In certain embodiments, the ds oligonucleotide comprises a guide strand backbone phosphoryl guanidine chiral center in the Sp configuration between the +3 nucleotide and the immediately downstream (+4) nucleotide, i.e., in the 3′ direction, and the guide strand further comprises one or more Rp, Sp, or stereorandom non-negatively charged internucleotidic linkage occurs between the second (+2) and third (+3) nucleotides, relative to the 5′ terminal nucleotide, of the guide strand and the internucleotidic linkage to the penultimate 3′ (N-1) nucleotide, and one or more of.
(1) a guide strand where one or both of the 5′ and 3′ terminal dinucleotides are not linked by non-negatively charged internucleotidic linkages, i.e., the guide strand comprises one more non-negatively charged internucleotidic linkages downstream, i.e., in the 3′ direction, relative to the linkage between the 5′ terminal dinucleotide and/or upstream, i.e., in the 5′ direction, relative to the linkage between the 3′ terminal dinucleotide;
(2) a guide strand where one or more Rp, Sp, or stereorandom non-negatively charged internucleotidic linkage occurs between any two adjacent nucleotides between the second (+2) nucleotide relative to the 5′ terminal nucleotide of the guide strand and the penultimate 3′ (N-1) nucleotide of the guide strand, where N is the 3′ terminal nucleotide;
(3) a guide strand where an Rp, Sp, or stereorandom non-negatively charged internucleotidic linkage occurs between the tenth (+10) and eleventh (+11) nucleotides, relative to the 5′ terminal nucleotide;
(4) a guide strand where an Rp, Sp, or stereorandom non-negatively charged internucleotidic linkage occurs between the seventh (+7) and eighth (+8) nucleotides, relative to the 5′ terminal nucleotide;
(5) a guide strand comprising one or more backbone phosphorothioate chiral centers in Rp or Sp configuration where linkage occurs between any two adjacent nucleotides between the penultimate 3′ nucleotide of the guide strand, where N is the 3′ terminal nucleotide, and the upstream N-10 nucleotide;
(6) a passenger strand where one or more Rp, Sp, or stereorandom non-negatively charged internucleotidic linkage occurs upstream, i.e., in the 5′ direction, relative to the central nucleotide of the passenger strand;
(7) a passenger strand where one or more Rp, Sp, or stereorandom non-negatively charged internucleotidic linkage occurs downstream, i.e., in the 3′ direction, relative to the central nucleotide of the passenger strand;
(8) a passenger strand where one or more backbone phosphorothioate chiral centers in Rp or Sp configuration internucleotidic linkage occurs upstream, i.e., in the 5′ direction, relative to the central nucleotide of the passenger strand;
(9) a passenger strand where one or more backbone phosphorothioate chiral centers in Rp or Sp configuration internucleotidic linkage internucleotidic linkage occurs downstream, i.e., in the 3′ direction, relative to the central nucleotide of the passenger strand; and
(10) a passenger strand comprising one or more modified sugars, e.g. Homo-DNA, between the 5′ terminal (+1) nucleotide and the penultimate (N-1) nucleotide.
   In certain embodiments, the ds oligonucleotide further comprises a 2′ modification, e.g., a 2′ F modification, of the 3′ nucleotide of a nucleotide pair linked by an Rp, Sp, or stereorandom non-negatively charged internucleotidic linkage, and the passenger strand comprises 0-n Rp, Sp, or stereorandom non-negatively charged internucleotidic linkages, where n is about 1 to 49.

In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, further comprise one or a plurality of modified sugars. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the 5′ terminal (+1) nucleotide, second (+2) nucleotide, third (+3) nucleotide, fourth (+4) nucleotide, fifth (+5) nucleotide, sixth (+6) nucleotide, seventh (+7) nucleotide, eighth (+8) nucleotide, ninth (+9) nucleotide, tenth (+10) nucleotide, twelfth (+12) nucleotide, sixteenth (+16) nucleotide, seventeenth (+17) nucleotide, eighteenth (+18) nucleotide, nineteenth (+19) nucleotide, twentieth (+20) nucleotide, twenty-first (+21) nucleotide, twenty-second (+22), and/or twenty-third (+23), the 3′ terminal, nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the 5′ terminal (+1) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the second (+2) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the third (+3) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the fourth (+4) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the fifth (+5) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the sixth (+6) nucleotide. In certain embodiments, the modified sugar comprises a 2′-F modification, 2′-H modification, 2′-OH modification, 2′-O-alkyl modification, e.g., 2′-O-methyl (OMe) modification, 2′-methoxyethyl (MOE) modification, 5′-alkyl modification, e.g., 5′-(R)-methyl or 5′-(S)-methyl, DNA, locked nucleic acid (LNA), unlocked nucleic acid (UNA), glycol nucleic acid (GNA), or Homo-DNA. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the 5′ terminal (+1) nucleotide, second (+2) nucleotide, third (+3) nucleotide, fourth (+4) nucleotide, fifth (+5) nucleotide, and/or sixth (+6) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the 5′ terminal (+1) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the second (+2) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the third (+3) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the fourth (+4) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the fifth (+5) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the sixth (+6) nucleotide.

In certain embodiments, the one or more Rp, Sp, or stereorandom non-negatively charged internucleotidic linkage incorporated into the guide or passenger strand is an Rp non-negatively charged internucleotidic linkage. In certain embodiments, the one or more Rp, Sp, or stereorandom non-negatively charged internucleotidic linkage is an Sp non-negatively charged internucleotidic linkage. In certain embodiments, the one or more Rp, Sp, or stereorandom non-negatively charged internucleotidic linkage is a stereorandom non-negatively charged internucleotidic linkage. In certain embodiments, the passenger strand comprises an Sp backbone phosphorothioate chiral center between the 5′ terminal (+1) nucleotide and the immediately downstream (+2) nucleotide and an Sp backbone phosphorothioate chiral center between the penultimate (N-1) nucleotide and the 3′ terminal (N) nucleotide.

In certain embodiments, the ds oligonucleotide comprises a guide strand backbone phosphoryl guanidine chiral center in the Sp configuration between the +3 nucleotide and the immediately downstream (+4) nucleotide, i.e., in the 3′ direction, and the guide strand further comprises backbone phosphorothioate chiral centers in Sp configuration between the 3′ terminal nucleotide and the penultimate (N-1) nucleotide and as between the penultimate (N-1) nucleotide and the immediately upstream (N-2) nucleotide, and one or more of
(1) a guide strand where one or both of the 5′ and 3′ terminal dinucleotides are not linked by non-negatively charged internucleotidic linkages, i.e., the guide strand comprises one more non-negatively charged internucleotidic linkages downstream, i.e., in the 3′ direction, relative to the linkage between the 5′ terminal dinucleotide and/or upstream, i.e., in the 5′ direction, relative to the linkage between the 3′ terminal dinucleotide;
(2) a guide strand where one or more Rp, Sp, or stereorandom non-negatively charged internucleotidic linkage occurs between any two adjacent nucleotides between the second (+2) nucleotide relative to the 5′ terminal nucleotide of the guide strand and the penultimate 3′ (N-1) nucleotide of the guide strand, where N is the 3′ terminal nucleotide;
(3) a guide strand where an Rp, Sp, or stereorandom non-negatively charged internucleotidic linkage occurs between the tenth (+10) and eleventh (+11) nucleotides, relative to the 5′ terminal nucleotide;
(4) a guide strand where an Rp, Sp, or stereorandom non-negatively charged internucleotidic linkage occurs between the seventh (+7) and eighth (+8) nucleotides, relative to the 5′ terminal nucleotide;
(5) a guide strand comprising one or more backbone phosphorothioate chiral centers in Rp or Sp configuration where linkage occurs between any two adjacent nucleotides between the penultimate 3′ nucleotide of the guide strand, where N is the 3′ terminal nucleotide, and the upstream N-10 nucleotide;
(6) a passenger strand where one or more Rp, Sp, or stereorandom non-negatively charged internucleotidic linkage occurs upstream, i.e., in the 5′ direction, relative to the central nucleotide of the passenger strand;
(7) a passenger strand where one or more Rp, Sp, or stereorandom non-negatively charged internucleotidic linkage occurs downstream, i.e., in the 3′ direction, relative to the central nucleotide of the passenger strand;
(8) a passenger strand where one or more backbone phosphorothioate chiral centers in Rp or Sp configuration internucleotidic linkage occurs upstream, i.e., in the 5′ direction, relative to the central nucleotide of the passenger strand;
(9) a passenger strand where one or more backbone phosphorothioate chiral centers in Rp or Sp configuration internucleotidic linkage internucleotidic linkage occurs downstream, i.e., in the 3′ direction, relative to the central nucleotide of the passenger strand; and
(10) a passenger strand comprising one or more modified sugars, e.g. Homo-DNA, between the 5′ terminal (+1) nucleotide and the penultimate (N-1) nucleotide.
   In certain embodiments, the ds oligonucleotide further comprises a 2′ modification, e.g., a 2′ F modification, of the 3′ nucleotide of a nucleotide pair linked by an Rp, Sp, or stereorandom non-negatively charged internucleotidic linkage, and the passenger strand comprises one or more backbone phosphorothioate chiral centers in Rp or Sp configuration. In certain embodiments, the one or more Rp, Sp, or stereorandom non-negatively charged internucleotidic linkage incorporated into the guide or passenger strand is an Rp non-negatively charged internucleotidic linkage. In certain embodiments, the one or more Rp, Sp, or stereorandom non-negatively charged internucleotidic linkage is an Sp non-negatively charged internucleotidic linkage. In certain embodiments, the one or more Rp, Sp, or stereorandom non-negatively charged internucleotidic linkage is a stereorandom non-negatively charged internucleotidic linkage. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, further comprise one or a plurality of modified sugars. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the 5′ terminal (+1) nucleotide, second (+2) nucleotide, third (+3) nucleotide, fourth (+4) nucleotide, fifth (+5) nucleotide, sixth (+6) nucleotide, seventh (+7) nucleotide, eighth (+8) nucleotide, ninth (+9) nucleotide, tenth (+10) nucleotide, twelfth (+12) nucleotide, sixteenth (+16) nucleotide, seventeenth (+17) nucleotide, eighteenth (+18) nucleotide, nineteenth (+19) nucleotide, twentieth (+20) nucleotide, twenty-first (+21) nucleotide, twenty-second (+22), and/or twenty-third (+23), the 3′ terminal, nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the 5′ terminal (+1) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the second (+2) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the third (+3) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the fourth (+4) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the fifth (+5) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the sixth (+6) nucleotide. In certain embodiments, the modified sugar comprises a 2′-F modification, 2′-H modification, 2′-OH modification, 2′-O-alkyl modification, e.g., 2′-O-methyl (OMe) modification, 2′-methoxyethyl (MOE) modification, 5′-alkyl modification, e.g., 5′-(R)-methyl or 5′-(S)-methyl, DNA, locked nucleic acid (LNA), unlocked nucleic acid (UNA), glycol nucleic acid (GNA), or Homo-DNA. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the 5′ terminal (+1) nucleotide, second (+2) nucleotide, third (+3) nucleotide, fourth (+4) nucleotide, fifth (+5) nucleotide, and/or sixth (+6) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the 5′ terminal (+1) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the second (+2) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the third (+3) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the fourth (+4) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the fifth (+5) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the sixth (+6) nucleotide.

In certain embodiments, the ds oligonucleotide comprises a guide strand backbone phosphoryl guanidine chiral center in the Sp configuration between the +3 nucleotide and the immediately downstream (+4) nucleotide, i.e., in the 3′ direction, and the guide strand comprises backbone phosphorothioate chiral centers in Rp, Sp, or alternating configurations between the 5′ terminal (+1) nucleotide and the immediately downstream (+2) nucleotide and between the +2 nucleotide and the immediately downstream (+3) nucleotide, and one or more of
(1) a guide strand where one or both of the 5′ and 3′ terminal dinucleotides are not linked by non-negatively charged internucleotidic linkages, i.e., the guide strand comprises one more non-negatively charged internucleotidic linkages downstream, i.e., in the 3′ direction, relative to the linkage between the 5′ terminal dinucleotide and/or upstream, i.e., in the 5′ direction, relative to the linkage between the 3′ terminal dinucleotide;
(2) a guide strand where one or more Rp, Sp, or stereorandom non-negatively charged internucleotidic linkage occurs between any two adjacent nucleotides between the second (+2) nucleotide relative to the 5′ terminal nucleotide of the guide strand and the penultimate 3′ (N-1) nucleotide of the guide strand, where N is the 3′ terminal nucleotide;
(3) a guide strand where an Rp, Sp, or stereorandom non-negatively charged internucleotidic linkage occurs between the tenth (+10) and eleventh (+11) nucleotides, relative to the 5′ terminal nucleotide;
(4) a guide strand where an Rp, Sp, or stereorandom non-negatively charged internucleotidic linkage occurs between the seventh (+7) and eighth (+8) nucleotides, relative to the 5′ terminal nucleotide;
(5) a guide strand comprising one or more backbone phosphorothioate chiral centers in Rp or Sp configuration where linkage occurs between any two adjacent nucleotides between the penultimate 3′ nucleotide of the guide strand, where N is the 3′ terminal nucleotide, and the upstream N-10 nucleotide;
(6) a passenger strand where one or more Rp, Sp, or stereorandom non-negatively charged internucleotidic linkage occurs upstream, i.e., in the 5′ direction, relative to the central nucleotide of the passenger strand;
(7) a passenger strand where one or more Rp, Sp, or stereorandom non-negatively charged internucleotidic linkage occurs downstream, i.e., in the 3′ direction, relative to the central nucleotide of the passenger strand;
(8) a passenger strand where one or more backbone phosphorothioate chiral centers in Rp or Sp configuration internucleotidic linkage occurs upstream, i.e., in the 5′ direction, relative to the central nucleotide of the passenger strand;
(9) a passenger strand where one or more backbone phosphorothioate chiral centers in Rp or Sp configuration internucleotidic linkage internucleotidic linkage occurs downstream, i.e., in the 3′ direction, relative to the central nucleotide of the passenger strand; and
(10) a passenger strand comprising one or more modified sugars, e.g. Homo-DNA, between the 5′ terminal (+1) nucleotide and the penultimate (N-1) nucleotide.
   wherein the ds oligonucleotide further comprises a 2′ modification, e.g., a 2′ F modification, of the 3′ nucleotide of a nucleotide pair linked by an Rp, Sp, or stereorandom-negatively charged internucleotidic linkage, and the passenger strand comprises one or more backbone chiral centers in Rp or Sp configuration. In certain embodiments, the one or more Rp, Sp, or stereorandom non-negatively charged internucleotidic linkage incorporated into the guide or passenger strand is an Rp non-negatively charged internucleotidic linkage. In certain embodiments, the one or more Rp, Sp, or stereorandom non-negatively charged internucleotidic linkage is an Sp non-negatively charged internucleotidic linkage. In certain embodiments, the one or more Rp, Sp, or stereorandom non-negatively charged internucleotidic linkage is a stereorandom non-negatively charged internucleotidic linkage. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, further comprise one or a plurality of modified sugars. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the 5′ terminal (+1) nucleotide, second (+2) nucleotide, third (+3) nucleotide, fourth (+4) nucleotide, fifth (+5) nucleotide, sixth (+6) nucleotide, seventh (+7)nucleotide, eighth (+8) nucleotide, ninth (+9) nucleotide, tenth (+10) nucleotide, twelfth (+12) nucleotide, sixteenth (+16) nucleotide, seventeenth (+17) nucleotide, eighteenth (+18) nucleotide, nineteenth (+19) nucleotide, twentieth (+20) nucleotide, twenty-first (+21) nucleotide, twenty-second (+22), and/or twenty-third (+23), the 3′ terminal, nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the 5′ terminal (+1) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the second (+2) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the third (+3) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the fourth (+4) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the fifth (+5) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the sixth (+6) nucleotide. In certain embodiments, the modified sugar comprises a 2′-F modification, 2′-H modification, 2′-OH modification, 2′-O-alkyl modification, e.g., 2′-O-methyl (OMe) modification, 2′-methoxyethyl (MOE) modification, 5′-alkyl modification, e.g., 5′-(R)-methyl or 5′-(S)-methyl, DNA, locked nucleic acid (LNA), unlocked nucleic acid (UNA), glycol nucleic acid (GNA), or Homo-DNA. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the 5′ terminal (+1) nucleotide, second (+2) nucleotide, third (+3) nucleotide, fourth (+4) nucleotide, fifth (+5) nucleotide, and/or sixth (+6) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the 5′ terminal (+1) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the second (+2) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the third (+3) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the fourth (+4) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the fifth (+5) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the sixth (+6) nucleotide. In certain embodiments, the ds oligonucleotide comprises a guide strand backbone phosphoryl guanidine chiral center in the Sp configuration between the +3 nucleotide and the immediately downstream (+4) nucleotide, i.e., in the 3′ direction, and the guide strand further comprises one or more backbone phosphorothioate chiral centers in Rp or Sp configuration upstream of backbone chiral centers in Sp configuration between the 3′ terminal nucleotide and the penultimate (N-1) nucleotide and as between the penultimate (N-1) nucleotide and the immediately upstream (N-2) nucleotide, and one or more of:
(1) a guide strand where one or both of the 5′ and 3′ terminal dinucleotides are not linked by non-negatively charged internucleotidic linkages, i.e., the guide strand comprises one more non-negatively charged internucleotidic linkages downstream, i.e., in the 3′ direction, relative to the linkage between the 5′ terminal dinucleotide and/or upstream, i.e., in the 5′ direction, relative to the linkage between the 3′ terminal dinucleotide;
(2) a guide strand where one or more Rp, Sp, or stereorandom non-negatively charged internucleotidic linkage occurs between any two adjacent nucleotides between the second (+2) nucleotide relative to the 5′ terminal nucleotide of the guide strand and the penultimate 3′ (N-1) nucleotide of the guide strand, where N is the 3′ terminal nucleotide;
(3) a guide strand where an Rp, Sp, or stereorandom non-negatively charged internucleotidic linkage occurs between the tenth (+10) and eleventh (+11) nucleotides, relative to the 5′ terminal nucleotide;
(4) a guide strand where an Rp, Sp, or stereorandom non-negatively charged internucleotidic linkage occurs between the seventh (+7) and eighth (+8) nucleotides, relative to the 5′ terminal nucleotide;
(5) a guide strand comprising one or more backbone phosphorothioate chiral centers in Rp or Sp configuration where linkage occurs between any two adjacent nucleotides between the penultimate 3′ nucleotide of the guide strand, where N is the 3′ terminal nucleotide, and the upstream N-10 nucleotide;
(6) a passenger strand where one or more Rp, Sp, or stereorandom non-negatively charged internucleotidic linkage occurs upstream, i.e., in the 5′ direction, relative to the central nucleotide of the passenger strand;
(7) a passenger strand where one or more Rp, Sp, or stereorandom non-negatively charged internucleotidic linkage occurs downstream, i.e., in the 3′ direction, relative to the central nucleotide of the passenger strand;
(8) a passenger strand where one or more backbone phosphorothioate chiral centers in Rp or Sp configuration internucleotidic linkage occurs upstream, i.e., in the 5′ direction, relative to the central nucleotide of the passenger strand;
(9) a passenger strand where one or more backbone phosphorothioate chiral centers in Rp or Sp configuration internucleotidic linkage internucleotidic linkage occurs downstream, i.e., in the 3′ direction, relative to the central nucleotide of the passenger strand; and
(10) a passenger strand comprising one or more modified sugars, e.g. Homo-DNA, between the 5′ terminal (+1) nucleotide and the penultimate (N-1) nucleotide.

In certain embodiments, the ds oligonucleotide further comprises a 2′ modification, e.g., a 2′ F modification, of the 3′ nucleotide of a nucleotide pair linked by an Rp, Sp, or stereorandom non-negatively charged internucleotidic linkage, and the passenger strand comprises one or more backbone chiral centers in Rp or Sp configuration. In certain embodiments, the one or more Rp, Sp, or stereorandom non-negatively charged internucleotidic linkage incorporated into the guide or passenger strand is an Rp non-negatively charged internucleotidic linkage. In certain embodiments, the one or more Rp, Sp, or stereorandom non-negatively charged internucleotidic linkage is an Sp non-negatively charged internucleotidic linkage. In certain embodiments, the one or more Rp, Sp, or stereorandom non-negatively charged internucleotidic linkage is a stereorandom non-negatively charged internucleotidic linkage. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, further comprise one or a plurality of modified sugars. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the 5′ terminal (+1) nucleotide, second (+2) nucleotide, third (+3) nucleotide, fourth (+4) nucleotide, fifth (+5) nucleotide, sixth (+6) nucleotide, seventh (+7) nucleotide, eighth (+8) nucleotide, ninth (+9) nucleotide, tenth (+10) nucleotide, twelfth (+12) nucleotide, sixteenth (+16) nucleotide, seventeenth (+17) nucleotide, eighteenth (+18) nucleotide, nineteenth (+19) nucleotide, twentieth (+20) nucleotide, twenty-first (+21) nucleotide, twenty-second (+22), and/or twenty-third (+23), the 3′ terminal, nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the 5′ terminal (+1) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the second (+2) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the third (+3) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the fourth (+4) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the fifth (+5) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the sixth (+6) nucleotide. In certain embodiments, the modified sugar comprises a 2′-F modification, 2′-H modification, 2′-OH modification, 2′-O-alkyl modification, e.g., 2′-O-methyl (OMe) modification, 2′-methoxyethyl (MOE) modification, 5′-alkyl modification, e.g., 5′-(R)-methyl or 5′-(S)-methyl, DNA, locked nucleic acid (LNA), unlocked nucleic acid (UNA), glycol nucleic acid (GNA), or Homo-DNA. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the 5′ terminal (+1) nucleotide, second (+2) nucleotide, third (+3) nucleotide, fourth (+4) nucleotide, fifth (+5) nucleotide, and/or sixth (+6) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the 5′ terminal (+1) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the second (+2) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the third (+3) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the fourth (+4) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the fifth (+5) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the sixth (+6) nucleotide.

In certain embodiments, the ds oligonucleotide comprises a guide strand backbone phosphoryl guanidine chiral center in the Sp configuration between the +3 nucleotide and the immediately downstream (+4) nucleotide, i.e., in the 3′ direction, and the guide strand further comprises one or more backbone phosphorothioate chiral centers in Rp or Sp configuration where linkage occurs between any two adjacent nucleotides between the penultimate 3′ nucleotide of the guide strand, where N is the 3′ terminal nucleotide, and the upstream N-10 nucleotide, and one or more of.
(1) a guide strand where one or both of the 5′ and 3′ terminal dinucleotides are not linked by non-negatively charged internucleotidic linkages, i.e., the guide strand comprises one more non-negatively charged internucleotidic linkages downstream, i.e., in the 3′ direction, relative to the linkage between the 5′ terminal dinucleotide and/or upstream, i.e., in the 5′ direction, relative to the linkage between the 3′ terminal dinucleotide;
(2) a guide strand where one or more Rp, Sp, or stereorandom non-negatively charged internucleotidic linkage occurs between any two adjacent nucleotides between the second (+2) nucleotide relative to the 5′ terminal nucleotide of the guide strand and the penultimate 3′ (N-1) nucleotide of the guide strand, where N is the 3′ terminal nucleotide;
(3) a guide strand where an Rp, Sp, or stereorandom non-negatively charged internucleotidic linkage occurs between the tenth (+10) and eleventh (+11) nucleotides, relative to the 5′ terminal nucleotide;
(4) a guide strand where an Rp, Sp, or stereorandom non-negatively charged internucleotidic linkage occurs between the seventh (+7) and eighth (+8) nucleotides, relative to the 5′ terminal nucleotide;
(5) a guide strand comprising one or more backbone phosphorothioate chiral centers in Rp or Sp configuration where linkage occurs between any two adjacent nucleotides between the penultimate 3′ nucleotide of the guide strand, where N is the 3′ terminal nucleotide, and the upstream N-10 nucleotide;
(6) a passenger strand where one or more Rp, Sp, or stereorandom non-negatively charged internucleotidic linkage occurs upstream, i.e., in the 5′ direction, relative to the central nucleotide of the passenger strand;
(7) a passenger strand where one or more Rp, Sp, or stereorandom non-negatively charged internucleotidic linkage occurs downstream, i.e., in the 3′ direction, relative to the central nucleotide of the passenger strand;
(8) a passenger strand where one or more backbone phosphorothioate chiral centers in Rp or Sp configuration internucleotidic linkage occurs upstream, i.e., in the 5′ direction, relative to the central nucleotide of the passenger strand;
(9) a passenger strand where one or more backbone phosphorothioate chiral centers in Rp or Sp configuration internucleotidic linkage internucleotidic linkage occurs downstream, i.e., in the 3′ direction, relative to the central nucleotide of the passenger strand; and
(10) a passenger strand comprising one or more modified sugars, e.g. Homo-DNA, between the 5′ terminal (+1) nucleotide and the penultimate (N-1) nucleotide.
   In certain embodiments, the ds oligonucleotide further comprises a 2′ modification, e.g., a 2′ F modification, of the 3′ nucleotide of a nucleotide pair linked by an Rp, Sp, or stereorandom non-negatively charged internucleotidic linkage, and the passenger strand comprises one or more backbone chiral centers in Rp or Sp configuration. In certain embodiments, the one or more Rp, Sp, or stereorandom non-negatively charged internucleotidic linkage incorporated into the guide or passenger strand is an Rp non-negatively charged internucleotidic linkage. In certain embodiments, the one or more Rp, Sp, or stereorandom non-negatively charged internucleotidic linkage is an Sp non-negatively charged internucleotidic linkage. In certain embodiments, the one or more Rp, Sp, or stereorandom non-negatively charged internucleotidic linkage is a stereorandom non-negatively charged internucleotidic linkage. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, further comprise one or a plurality of modified sugars. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the 5′ terminal (+1) nucleotide, second (+2) nucleotide, third (+3) nucleotide, fourth (+4) nucleotide, fifth (+5) nucleotide, sixth (+6) nucleotide, seventh (+7) nucleotide, eighth (+8) nucleotide, ninth (+9) nucleotide, tenth (+10) nucleotide, twelfth (+12) nucleotide, sixteenth (+16) nucleotide, seventeenth (+17) nucleotide, eighteenth (+18) nucleotide, nineteenth (+19) nucleotide, twentieth (+20) nucleotide, twenty-first (+21) nucleotide, twenty-second (+22), and/or twenty-third (+23), the 3′ terminal, nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the 5′ terminal (+1) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the second (+2) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the third (+3) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the fourth (+4) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the fifth (+5) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the sixth (+6) nucleotide. In certain embodiments, the modified sugar comprises a 2′-F modification, 2′-H modification, 2′-OH modification, 2′-O-alkyl modification, e.g., 2′-O-methyl (OMe) modification, 2′-methoxyethyl (MOE) modification, 5′-alkyl modification, e.g., 5′-(R)-methyl or 5′-(S)-methyl, DNA, locked nucleic acid (LNA), unlocked nucleic acid (UNA), glycol nucleic acid (GNA), or Homo-DNA. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the 5′ terminal (+1) nucleotide, second (+2) nucleotide, third (+3) nucleotide, fourth (+4) nucleotide, fifth (+5) nucleotide, and/or sixth (+6) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the 5′ terminal (+1) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the second (+2) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the third (+3) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the fourth (+4) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the fifth (+5) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the sixth (+6) nucleotide

In certain embodiments, the ds oligonucleotide comprises a guide strand backbone phosphoryl guanidine chiral center in the Sp configuration between the +3 nucleotide and the immediately downstream (+4) nucleotide, i.e., in the 3′ direction, and the guide strand further comprises a backbone phosphoryl guanidine chiral center in the Sp configuration between the +7 and the immediately downstream (+8) and one or more of.
(1) a guide strand where one or both of the 5′ and 3′ terminal dinucleotides are not linked by non-negatively charged internucleotidic linkages, i.e., the guide strand comprises one more non-negatively charged internucleotidic linkages downstream, i.e., in the 3′ direction, relative to the linkage between the 5′ terminal dinucleotide and/or upstream, i.e., in the 5′ direction, relative to the linkage between the 3′ terminal dinucleotide;
(2) a guide strand where one or more Rp, Sp, or stereorandom non-negatively charged internucleotidic linkage occurs between any two adjacent nucleotides between the second (+2) nucleotide relative to the 5′ terminal nucleotide of the guide strand and the penultimate 3′ (N-1) nucleotide of the guide strand, where N is the 3′ terminal nucleotide;
(3) a guide strand where an Rp, Sp, or stereorandom non-negatively charged internucleotidic linkage occurs between the tenth (+10) and eleventh (+11) nucleotides, relative to the 5′ terminal nucleotide;
(4) a guide strand where an Rp, Sp, or stereorandom non-negatively charged internucleotidic linkage occurs between the seventh (+7) and eighth (+8) nucleotides, relative to the 5′ terminal nucleotide;
(5) a guide strand comprising one or more backbone phosphorothioate chiral centers in Rp or Sp configuration where linkage occurs between any two adjacent nucleotides between the penultimate 3′ nucleotide of the guide strand, where N is the 3′ terminal nucleotide, and the upstream N-10 nucleotide;
(6) a passenger strand where one or more Rp, Sp, or stereorandom non-negatively charged internucleotidic linkage occurs upstream, i.e., in the 5′ direction, relative to the central nucleotide of the passenger strand;
(7) a passenger strand where one or more Rp, Sp, or stereorandom non-negatively charged internucleotidic linkage occurs downstream, i.e., in the 3′ direction, relative to the central nucleotide of the passenger strand;
(8) a passenger strand where one or more backbone phosphorothioate chiral centers in Rp or Sp configuration internucleotidic linkage occurs upstream, i.e., in the 5′ direction, relative to the central nucleotide of the passenger strand;
(9) a passenger strand where one or more backbone phosphorothioate chiral centers in Rp or Sp configuration internucleotidic linkage internucleotidic linkage occurs downstream, i.e., in the 3′ direction, relative to the central nucleotide of the passenger strand; and
(10) a passenger strand comprising one or more modified sugars, e.g. Homo-DNA, between the 5′ terminal (+1) nucleotide and the penultimate (N-1) nucleotide.
   In certain embodiments, the ds oligonucleotide further comprises a 2′ modification, e.g., a 2′ F modification, of the 3′ nucleotide of a nucleotide pair linked by an Rp, Sp, or stereorandom non-negatively charged internucleotidic linkage, and the passenger strand comprises one or more backbone chiral centers in Rp or Sp configuration. In certain embodiments, the one or more Rp, Sp, or stereorandom non-negatively charged internucleotidic linkage incorporated into the guide or passenger strand is an Rp non-negatively charged internucleotidic linkage. In certain embodiments, the one or more Rp, Sp, or stereorandom non-negatively charged internucleotidic linkage is an Sp non-negatively charged internucleotidic linkage. In certain embodiments, the one or more Rp, Sp, or stereorandom non-negatively charged internucleotidic linkage is a stereorandom non-negatively charged internucleotidic linkage. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, further comprise one or a plurality of modified sugars. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the 5′ terminal (+1) nucleotide, second (+2) nucleotide, third (+3) nucleotide, fourth (+4) nucleotide, fifth (+5) nucleotide, sixth (+6) nucleotide, seventh (+7) nucleotide, eighth (+8) nucleotide, ninth (+9) nucleotide, tenth (+10) nucleotide, twelfth (+12) nucleotide, sixteenth (+16) nucleotide, seventeenth (+17) nucleotide, eighteenth (+18) nucleotide, nineteenth (+19) nucleotide, twentieth (+20) nucleotide, twenty-first (+21) nucleotide, twenty-second (+22), and/or twenty-third (+23), the 3′ terminal, nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the 5′ terminal (+1) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the second (+2) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the third (+3) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the fourth (+4) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the fifth (+5) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the sixth (+6) nucleotide. In certain embodiments, the modified sugar comprises a 2′-F modification, 2′-H modification, 2′-OH modification, 2′-O-alkyl modification, e.g., 2′-O-methyl (OMe) modification, 2′-methoxyethyl (MOE) modification, 5′-alkyl modification, e.g., 5′-(R)-methyl or 5′-(S)-methyl, DNA, locked nucleic acid (LNA), unlocked nucleic acid (UNA), glycol nucleic acid (GNA), or Homo-DNA. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the 5′ terminal (+1) nucleotide, second (+2) nucleotide, third (+3) nucleotide, fourth (+4) nucleotide, fifth (+5) nucleotide, and/or sixth (+6) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the 5′ terminal (+1) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the second (+2) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the third (+3) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the fourth (+4) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the fifth (+5) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the sixth (+6) nucleotide

In certain embodiments, the ds oligonucleotide comprises a guide strand backbone phosphoryl guanidine chiral center in the Sp configuration between the +3 nucleotide and the immediately downstream (+4) nucleotide, i.e., in the 3′ direction, and the guide strand further comprises one or more backbone phosphorothioate chiral centers in Rp or Sp configuration between the 5′ terminal (+1)nucleotide and the immediately downstream (+2) nucleotide and between the (+2) nucleotide and the immediately downstream (+3) nucleotide, as well as between the (+5) nucleotide and the (+6) nucleotide, and one or more of:
(1) a guide strand where one or both of the 5′ and 3′ terminal dinucleotides are not linked by non-negatively charged internucleotidic linkages, i.e., the guide strand comprises one more non-negatively charged internucleotidic linkages downstream, i.e., in the 3′ direction, relative to the linkage between the 5′ terminal dinucleotide and/or upstream, i.e., in the 5′ direction, relative to the linkage between the 3′ terminal dinucleotide;
(2) a guide strand where one or more Rp, Sp, or stereorandom non-negatively charged internucleotidic linkage occurs between any two adjacent nucleotides between the second (+2) nucleotide relative to the 5′ terminal nucleotide of the guide strand and the penultimate 3′ (N-1) nucleotide of the guide strand, where N is the 3′ terminal nucleotide;
(3) a guide strand where an Rp, Sp, or stereorandom non-negatively charged internucleotidic linkage occurs between the tenth (+10) and eleventh (+11) nucleotides, relative to the 5′ terminal nucleotide;
(4) a guide strand where an Rp, Sp, or stereorandom non-negatively charged internucleotidic linkage occurs between the seventh (+7) and eighth (+8) nucleotides, relative to the 5′ terminal nucleotide;
(5) a guide strand comprising one or more backbone phosphorothioate chiral centers in Rp or Sp configuration where linkage occurs between any two adjacent nucleotides between the penultimate 3′ nucleotide of the guide strand, where N is the 3′ terminal nucleotide, and the upstream N-10 nucleotide;
(6) a passenger strand where one or more Rp, Sp, or stereorandom non-negatively charged internucleotidic linkage occurs upstream, i.e., in the 5′ direction, relative to the central nucleotide of the passenger strand;
(7) a passenger strand where one or more Rp, Sp, or stereorandom non-negatively charged internucleotidic linkage occurs downstream, i.e., in the 3′ direction, relative to the central nucleotide of the passenger strand;
(8) a passenger strand where one or more backbone phosphorothioate chiral centers in Rp or Sp configuration internucleotidic linkage occurs upstream, i.e., in the 5′ direction, relative to the central nucleotide of the passenger strand;
(9) a passenger strand where one or more backbone phosphorothioate chiral centers in Rp or Sp configuration internucleotidic linkage internucleotidic linkage occurs downstream, i.e., in the 3′ direction, relative to the central nucleotide of the passenger strand; and
(10) a passenger strand comprising one or more modified sugars, e.g. Homo-DNA, between the 5′ terminal (+1) nucleotide and the penultimate (N-1) nucleotide.
   In certain embodiments, the ds oligonucleotide further comprises a 2′ modification, e.g., a 2′ F modification, of the 3′ nucleotide of a nucleotide pair linked by an Rp, Sp, or stereorandom non-negatively charged internucleotidic linkage, and the passenger strand comprises one or more backbone chiral centers in Rp or Sp configuration. In certain embodiments, the one or more Rp, Sp, or stereorandom non-negatively charged internucleotidic linkage incorporated into the guide or passenger strand is an Rp non-negatively charged internucleotidic linkage. In certain embodiments, the one or more Rp, Sp, or stereorandom non-negatively charged internucleotidic linkage is an Sp non-negatively charged internucleotidic linkage. In certain embodiments, the one or more Rp, Sp, or stereorandom non-negatively charged internucleotidic linkage is a stereorandom non-negatively charged internucleotidic linkage. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, further comprise one or a plurality of modified sugars. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the 5′ terminal (+1) nucleotide, second (+2) nucleotide, third (+3) nucleotide, fourth (+4) nucleotide, fifth (+5) nucleotide, sixth (+6) nucleotide, seventh (+7) nucleotide, eighth (+8) nucleotide, ninth (+9) nucleotide, tenth (+10) nucleotide, twelfth (+12) nucleotide, sixteenth (+16) nucleotide, seventeenth (+17) nucleotide, eighteenth (+18) nucleotide, nineteenth (+19) nucleotide, twentieth (+20) nucleotide, twenty-first (+21) nucleotide, twenty-second (+22), and/or twenty-third (+23), the 3′ terminal, nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the 5′ terminal (+1) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the second (+2) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the third (+3) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the fourth (+4) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the fifth (+5) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the sixth (+6) nucleotide. In certain embodiments, the modified sugar comprises a 2′-F modification, 2′-H modification, 2′-OH modification, 2′-O-alkyl modification, e.g., 2′-O-methyl (OMe) modification, 2′-methoxyethyl (MOE) modification, 5′-alkyl modification, e.g., 5′-(R)-methyl or 5′-(S)-methyl, DNA, locked nucleic acid (LNA), unlocked nucleic acid (UNA), glycol nucleic acid (GNA), or Homo-DNA. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the 5′ terminal (+1) nucleotide, second (+2) nucleotide, third (+3) nucleotide, fourth (+4) nucleotide, fifth (+5) nucleotide, and/or sixth (+6) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the 5′ terminal (+1) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the second (+2) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the third (+3) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the fourth (+4) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the fifth (+5) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the sixth (+6) nucleotide.

In certain embodiments, the ds oligonucleotide comprises a guide strand backbone phosphoryl guanidine chiral center in the Sp configuration between the +3 nucleotide and the immediately downstream (+4) nucleotide, i.e., in the 3′ direction, and the guide strand further comprises one or more Rp, Sp, or stereorandom non-negatively charged internucleotidic linkage occurs between any two adjacent nucleotides between the second (+2) nucleotide relative to the 5′ terminal nucleotide of the guide strand and the penultimate 3′ (N-1) nucleotide of the guide strand, where N is the 3′ terminal nucleotide, a 2′ modification, e.g., a 2′ F modification, of the 3′ nucleotide of a nucleotide pair linked by an Rp, Sp, or stereorandom non-negatively charged internucleotidic linkage, and the passenger strand comprises one or more backbone chiral centers in Rp or Sp configuration. In certain embodiments, the one or more Rp, Sp, or stereorandom non-negatively charged internucleotidic linkage incorporated into the guide strand is an Rp non-negatively charged internucleotidic linkage. In certain embodiments, the one or more Rp, Sp, or stereorandom non-negatively charged internucleotidic linkage is an Sp non-negatively charged internucleotidic linkage. In certain embodiments, the one or more Rp, Sp, or stereorandom non-negatively charged internucleotidic linkage is a stereorandom non-negatively charged internucleotidic linkage. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, further comprise one or a plurality of modified sugars. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the 5′ terminal (+1) nucleotide, second (+2) nucleotide, third (+3) nucleotide, fourth (+4) nucleotide, fifth (+5) nucleotide, sixth (+6) nucleotide, seventh (+7) nucleotide, eighth (+8) nucleotide, ninth (+9) nucleotide, tenth (+10) nucleotide, twelfth (+12) nucleotide, sixteenth (+16) nucleotide, seventeenth (+17) nucleotide, eighteenth (+18) nucleotide, nineteenth (+19) nucleotide, twentieth (+20) nucleotide, twenty-first (+21) nucleotide, twenty-second (+22), and/or twenty-third (+23), the 3′ terminal, nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the 5′ terminal (+1) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the second (+2) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the third (+3) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the fourth (+4) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the fifth (+5) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the sixth (+6) nucleotide. In certain embodiments, the modified sugar comprises a 2′-F modification, 2′-H modification, 2′-OH modification, 2′-O-alkyl modification, e.g., 2′-O-methyl (OMe) modification, 2′-methoxyethyl (MOE) modification, 5′-alkyl modification, e.g., 5′-(R)-methyl or 5′-(S)-methyl, DNA, locked nucleic acid (LNA), unlocked nucleic acid (UNA), glycol nucleic acid (GNA), or Homo-DNA. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the 5′ terminal (+1) nucleotide, second (+2) nucleotide, third (+3) nucleotide, fourth (+4) nucleotide, fifth (+5) nucleotide, and/or sixth (+6) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the 5′ terminal (+1) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the second (+2) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the third (+3) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the fourth (+4) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the fifth (+5) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the sixth (+6) nucleotide.

In certain embodiments, the ds oligonucleotide comprises a guide strand backbone phosphoryl guanidine chiral center in the Sp configuration between the +3 nucleotide and the immediately downstream (+4) nucleotide, i.e., in the 3′ direction, and the guide strand further comprises backbone phosphorothioate chiral centers in Sp configuration between the 3′ terminal nucleotide and the penultimate (N-1) nucleotide and as between the penultimate (N-1) nucleotide and the immediately upstream (N-2) nucleotide, a 2′ modification, e.g., a 2′ F modification, of the 3′ nucleotide of a nucleotide pair linked by an Rp, Sp, or stereorandom non-negatively charged internucleotidic linkage, and the passenger strand comprises 0-n Rp, Sp, or stereorandom non-negatively charged internucleotidic linkages, where n is about 1 to 49 and one or more backbone chiral centers in Rp or Sp configuration.

In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, further comprise one or a plurality of modified sugars. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the 5′ terminal (+1) nucleotide, second (+2) nucleotide, third (+3) nucleotide, fourth (+4) nucleotide, fifth (+5) nucleotide, sixth (+6) nucleotide, seventh (+7) nucleotide, eighth (+8) nucleotide, ninth (+9) nucleotide, tenth (+10) nucleotide, twelfth (+12) nucleotide, sixteenth (+16) nucleotide, seventeenth (+17) nucleotide, eighteenth (+18) nucleotide, nineteenth (+19) nucleotide, twentieth (+20) nucleotide, twenty-first (+21) nucleotide, twenty-second (+22), and/or twenty-third (+23), the 3′ terminal, nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the 5′ terminal (+1) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the second (+2) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the third (+3) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the fourth (+4) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the fifth (+5) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the sixth (+6) nucleotide. In certain embodiments, the modified sugar comprises a 2′-F modification, 2′-H modification, 2′-OH modification, 2′-O-alkyl modification, e.g., 2′-O-methyl (OMe) modification, 2′-methoxyethyl (MOE) modification, 5′-alkyl modification, e.g., 5′-(R)-methyl or 5′-(S)-methyl, DNA, locked nucleic acid (LNA), unlocked nucleic acid (UNA), glycol nucleic acid (GNA), or Homo-DNA. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the 5′ terminal (+1) nucleotide, second (+2) nucleotide, third (+3) nucleotide, fourth (+4) nucleotide, fifth (+5) nucleotide, and/or sixth (+6) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the 5′ terminal (+1) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the second (+2) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the third (+3) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the fourth (+4) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the fifth (+5) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the sixth (+6) nucleotide.

In certain embodiments, the one or more Rp, Sp, or stereorandom non-negatively charged internucleotidic linkage incorporated into the guide strand is an Rp non-negatively charged internucleotidic linkage. In certain embodiments, the one or more Rp, Sp, or stereorandom non-negatively charged internucleotidic linkage is an Sp non-negatively charged internucleotidic linkage. In certain embodiments, the one or more Rp, Sp, or stereorandom non-negatively charged internucleotidic linkage is a stereorandom non-negatively charged internucleotidic linkage. In certain further embodiments, the passenger strand comprises an Sp backbone phosphorothioate chiral center between the 5′ terminal (+1) nucleotide and the immediately downstream (+2) nucleotide and an Sp backbone phosphorothioate chiral center between the penultimate (N-1) nucleotide and the 3′ terminal (N) nucleotide.

In certain embodiments, the ds oligonucleotide comprises a guide strand backbone phosphoryl guanidine chiral center in the Sp configuration between the +3 nucleotide and the immediately downstream (+4) nucleotide, i.e., in the 3′ direction, and the guide strand further comprises backbone phosphorothioate chiral centers in Rp, Sp, or alternating configurations between the 5′ terminal (+1) nucleotide and the immediately downstream (+2) nucleotide and between the +2 nucleotide and the immediately downstream (+3) nucleotide, a 2′ modification, e.g., a 2′ F modification, of the 3′ nucleotide of a nucleotide pair linked by an Rp, Sp, or stereorandom non-negatively charged internucleotidic linkage, and the passenger strand comprises 0-n Rp, Sp, or stereorandom non-negatively charged internucleotidic linkages, where n is about 1 to 49 and one or more backbone chiral centers in Rp or Sp configuration.

In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, further comprise one or a plurality of modified sugars. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the 5′ terminal (+1) nucleotide, second (+2) nucleotide, third (+3) nucleotide, fourth (+4) nucleotide, fifth (+5) nucleotide, sixth (+6) nucleotide, seventh (+7) nucleotide, eighth (+8) nucleotide, ninth (+9) nucleotide, tenth (+10) nucleotide, twelfth (+12) nucleotide, sixteenth (+16) nucleotide, seventeenth (+17) nucleotide, eighteenth (+18) nucleotide, nineteenth (+19) nucleotide, twentieth (+20) nucleotide, twenty-first (+21) nucleotide, twenty-second (+22), and/or twenty-third (+23), the 3′ terminal, nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the 5′ terminal (+1) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the second (+2) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the third (+3) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the fourth (+4) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the fifth (+5) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the sixth (+6) nucleotide. In certain embodiments, the modified sugar comprises a 2′-F modification, 2′-H modification, 2′-OH modification, 2′-O-alkyl modification, e.g., 2′-O-methyl (OMe) modification, 2′-methoxyethyl (MOE) modification, 5′-alkyl modification, e.g., 5′-(R)-methyl or 5′-(S)-methyl, DNA, locked nucleic acid (LNA), unlocked nucleic acid (UNA), glycol nucleic acid (GNA), or Homo-DNA. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the 5′ terminal (+1) nucleotide, second (+2) nucleotide, third (+3) nucleotide, fourth (+4) nucleotide, fifth (+5) nucleotide, and/or sixth (+6) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the 5′ terminal (+1) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the second (+2) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the third (+3) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the fourth (+4) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the fifth (+5) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the sixth (+6) nucleotide.

In certain embodiments, the one or more Rp, Sp, or stereorandom non-negatively charged internucleotidic linkage incorporated into the guide strand is an Rp non-negatively charged internucleotidic linkage. In certain embodiments, the one or more Rp, Sp, or stereorandom non-negatively charged internucleotidic linkage is an Sp non-negatively charged internucleotidic linkage. In certain embodiments, the one or more Rp, Sp, or stereorandom non-negatively charged internucleotidic linkage is a stereorandom non-negatively charged internucleotidic linkage. In certain further embodiments, the passenger strand comprises an Sp backbone phosphorothioate chiral center between the 5′ terminal (+1) nucleotide and the immediately downstream (+2) nucleotide and an Sp backbone phosphorothioate chiral center between the penultimate (N-1) nucleotide and the 3′ terminal (N) nucleotide.

In certain embodiments, the ds oligonucleotide comprises a guide strand backbone phosphoryl guanidine chiral center in the Sp configuration between the +3 nucleotide and the immediately downstream (+4) nucleotide, i.e., in the 3′ direction, and the guide strand further comprises one or more backbone phosphorothioate chiral centers in Rp or Sp configuration upstream of backbone phosphorothioate chiral centers in Sp configuration between the 3′ terminal nucleotide and the penultimate (N-1) nucleotide and as between the penultimate (N-1) nucleotide and the immediately upstream (N-2) nucleotide, a 2′ modification, e.g., a 2′ F modification, of the 3′ nucleotide of a nucleotide pair linked by an Rp, Sp, or stereorandom non-negatively charged internucleotidic linkage, and the passenger strand comprises 0-n Rp, Sp, or stereorandom non-negatively charged internucleotidic linkages, where n is about 1 to 49 and one or more backbone chiral centers in Rp or Sp configuration.

In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, further comprise one or a plurality of modified sugars. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the 5′ terminal (+1) nucleotide, second (+2) nucleotide, third (+3) nucleotide, fourth (+4) nucleotide, fifth (+5) nucleotide, sixth (+6) nucleotide, seventh (+7) nucleotide, eighth (+8) nucleotide, ninth (+9) nucleotide, tenth (+10) nucleotide, twelfth (+12) nucleotide, sixteenth (+16) nucleotide, seventeenth (+17) nucleotide, eighteenth (+18) nucleotide, nineteenth (+19) nucleotide, twentieth. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the 5′ terminal (+1) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the second (+2) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the third (+3) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the fourth (+4) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the fifth (+5) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the sixth (+6) nucleotide. In certain embodiments, the modified sugar comprises a 2′-F modification, 2′-H modification, 2′-OH modification, 2′-O-alkyl modification, e.g., 2′-O-methyl (OMe) modification, 2′-methoxyethyl (MOE) modification, 5′-alkyl modification, e.g., 5′-(R)-methyl or 5′-(S)-methyl, DNA, locked nucleic acid (LNA), unlocked nucleic acid (UNA), glycol nucleic acid (GNA), or Homo-DNA. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the 5′ terminal (+1) nucleotide, second (+2) nucleotide, third (+3) nucleotide, fourth (+4) nucleotide, fifth (+5) nucleotide, and/or sixth (+6) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the 5′ terminal (+1) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the second (+2) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the third (+3) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the fourth (+4) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the fifth (+5) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the sixth (+6) nucleotide.

In certain embodiments, the one or more Rp, Sp, or stereorandom non-negatively charged internucleotidic linkage incorporated into the guide strand is an Rp non-negatively charged internucleotidic linkage. In certain embodiments, the one or more Rp, Sp, or stereorandom non-negatively charged internucleotidic linkage is an Sp non-negatively charged internucleotidic linkage. In certain embodiments, the one or more Rp, Sp, or stereorandom non-negatively charged internucleotidic linkage is a stereorandom non-negatively charged internucleotidic linkage. In certain further embodiments, the passenger strand comprises an Sp backbone phosphorothioate chiral center between the 5′ terminal (+1) nucleotide and the immediately downstream (+2) nucleotide and an Sp backbone phosphorothioate chiral center between the penultimate (N-1) nucleotide and the 3′ terminal (N) nucleotide.

In certain embodiments, the ds oligonucleotide comprises a guide strand backbone phosphoryl guanidine chiral center in the Sp configuration between the +3 nucleotide and the immediately downstream (+4) nucleotide, i.e., in the 3′ direction, and the guide strand further comprises one or more Rp, Sp, or stereorandom non-negatively charged internucleotidic linkage occurs between any two adjacent nucleotides between the second (+2) nucleotide relative to the 5′ terminal nucleotide of the guide strand and the penultimate 3′ (N-1) nucleotide of the guide strand, where N is the 3′ terminal nucleotide, a 2′ modification, e.g., a 2′ F modification, of the 3′ nucleotide of a nucleotide pair linked by an Rp, Sp, or stereorandom non-negatively charged internucleotidic linkage, and the passenger strand comprises 0-n Rp, Sp, or stereorandom non-negatively charged internucleotidic linkages, where n is about 1 to 49 and one or more backbone chiral centers in Rp or Sp configuration.

In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, further comprise one or a plurality of modified sugars. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the 5′ terminal (+1) nucleotide, second (+2) nucleotide, third (+3) nucleotide, fourth (+4) nucleotide, fifth (+5) nucleotide, sixth (+6) nucleotide, seventh (+7)nucleotide, eighth (+8) nucleotide, ninth (+9) nucleotide, tenth (+10) nucleotide, twelfth (+12) nucleotide, sixteenth (+16) nucleotide, seventeenth (+17) nucleotide, eighteenth (+18) nucleotide, nineteenth (+19) nucleotide, twentieth. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the 5′ terminal (+1) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the second (+2) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the third (+3) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the fourth (+4) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the fifth (+5) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the sixth (+6) nucleotide.

In certain embodiments, the modified sugar comprises a 2′-F modification, 2′-H modification, 2′-OH modification, 2′-O-alkyl modification, e.g., 2′-O-methyl (OMe) modification, 2′-methoxyethyl (MOE) modification, 5′-alkyl modification, e.g., 5′-(R)-methyl or 5′-(S)-methyl, DNA, locked nucleic acid (LNA), unlocked nucleic acid (UNA), glycol nucleic acid (GNA), or Homo-DNA.

In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the 5′ terminal (+1) nucleotide, second (+2) nucleotide, third (+3) nucleotide, fourth (+4) nucleotide, fifth (+5) nucleotide, and/or sixth (+6) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the 5′ terminal (+1) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the second (+2) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the third (+3) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the fourth (+4) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the fifth (+5) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the sixth (+6) nucleotide.

In certain embodiments, the ds oligonucleotides comprise a 5′-methyl modification of the third (+3) nucleotide and/or fourth (+4) nucleotide. In certain embodiments, the ds oligonucleotides comprises a 5′-alkyl modification of the third (+3) nucleotide. In certain embodiments, the ds oligonucleotides comprises a 5′-methyl modification of the third (+3) nucleotide. In certain embodiments, the ds oligonucleotides comprises a 5′-alkyl modification of the fourth (+4) nucleotide. In certain embodiments, the ds oligonucleotides comprises a 5′-methyl modification of the fourth (+4) nucleotide. In certain embodiments, the ds oligonucleotides comprises a 5′-alkyl modification of the third (+3) nucleotide and fourth (+4) nucleotide. In certain embodiments, the ds oligonucleotides comprises a 5′-methyl modification of the third (+3) nucleotide and fourth (+4) nucleotide.

In certain embodiments, the one or more Rp, Sp, or stereorandom non-negatively charged internucleotidic linkage incorporated into the guide strand is an Rp non-negatively charged internucleotidic linkage. In certain embodiments, the one or more Rp, Sp, or stereorandom non-negatively charged internucleotidic linkage is an Sp non-negatively charged internucleotidic linkage. In certain embodiments, the one or more Rp, Sp, or stereorandom non-negatively charged internucleotidic linkage is a stereorandom non-negatively charged internucleotidic linkage. In certain further embodiments, the passenger strand comprises an Sp backbone phosphorothioate chiral center between the 5′ terminal (+1) nucleotide and the immediately downstream (+2) nucleotide and an Sp backbone phosphorothioate chiral center between the penultimate (N-1) nucleotide and the 3′ terminal (N) nucleotide. In certain embodiments, provided ds oligonucleotides may participate in exon skipping mechanisms. In certain embodiments, provided ds oligonucleotides may be aptamers. In certain embodiments, provided ds oligonucleotides may bind to and inhibit the function of a protein, small molecule, nucleic acid or cell. In certain embodiments, provided ds oligonucleotides may participate in forming a triplex helix with a double-stranded nucleic acid in the cell. In certain embodiments, provided ds oligonucleotides may bind to genomic (e.g., chromosomal) nucleic acid. In certain embodiments, provided ds oligonucleotides may bind to genomic (e.g., chromosomal) nucleic acid, thus preventing or decreasing expression of the nucleic acid (e.g., by preventing or decreasing transcription, transcriptional enhancement, modification, etc.). In certain embodiments, provided ds oligonucleotides may bind to DNA quadruplexes. In certain embodiments, provided ds oligonucleotides may be immunomodulatory. In certain embodiments, provided ds oligonucleotides may be immunostimulatory. In certain embodiments, provided oligonucleotides may be immunostimulatory and may comprise a CpG sequence. In certain embodiments, provided ds oligonucleotides may be immunostimulatory and may comprise a CpG sequence and may be useful as an adjuvant. In certain embodiments, provided ds oligonucleotides may be immunostimulatory and may comprise a CpG sequence and may be useful as an adjuvant in treating a disease (e.g., an infectious disease or cancer). In certain embodiments, provided ds oligonucleotides may be therapeutic. In certain embodiments, provided ds oligonucleotides may be non-therapeutic. In certain embodiments, provided ds oligonucleotides may be therapeutic or non-therapeutic. In certain embodiments, provided ds oligonucleotides are useful in therapeutic, diagnostic, research and/or nanomaterials applications. In certain embodiments, provided ds oligonucleotides may be useful for experimental purposes. In certain embodiments, provided ds oligonucleotides may be useful for experimental purposes, e.g., as a probe, in a microarray, etc. In certain embodiments, provided ds oligonucleotides may participate in more than one biological mechanism; in certain such embodiments, for example, provided ds oligonucleotides may participate in both RNAi and RNase H mechanisms.

In certain embodiments, provided ds oligonucleotides are directed to a target (e.g., a target sequence, a target RNA, a target mRNA, a target pre-mRNA, a target gene, etc.). A target gene is a gene with respect to which expression and/or activity of one or more gene products (e.g., RNA and/or protein products) are intended to be altered. In certain embodiments, a target gene is intended to be inhibited. Thus, when a ds oligonucleotide as described herein acts on a particular target gene, presence and/or activity of one or more gene products of that gene are altered when the ds oligonucleotide is present as compared with when it is absent.

In certain embodiments, a target is a specific allele with respect to which expression and/or activity of one or more products (e.g., RNA and/or protein products) are intended to be altered. In certain embodiments, a target allele is one whose presence and/or expression is associated (e.g., correlated) with presence, incidence, and/or severity, of one or more diseases and/or conditions. Alternatively or additionally, in certain embodiments, a target allele is one for which alteration of level and/or activity of one or more gene products correlates with improvement (e.g., delay of onset, reduction of severity, responsiveness to other therapy, etc.) in one or more aspects of a disease and/or condition.

In certain embodiments, e.g., where presence and/or activity of a particular allele (a disease-associated allele) is associated (e.g., correlated) with presence, incidence and/or severity of one or more disorders, diseases and/or conditions, a different allele of the same gene exists and is not so associated, or is associated to a lesser extent (e.g., shows less significant, or statistically insignificant correlation), ds oligonucleotides and methods thereof as described herein may preferentially or specifically target the associated allele relative to the one or more less-associated/unassociated allele(s), thus mediating allele-specific suppression.

In certain embodiments, a target sequence is a sequence to which an oligonucleotide as described herein binds. In certain embodiments, a target sequence is identical to, or is an exact complement of, a sequence of a provided oligonucleotide, or of consecutive residues therein (e.g., a provided oligonucleotide includes a target-binding sequence that is identical to, or an exact complement of, a target sequence). In certain embodiments, a target-binding sequence is an exact complement of a target sequence of a transcript (e.g., pre-mRNA, mRNA, etc.). A target-binding sequence/target sequence can be of various lengths to provided oligonucleotides with desired activities and/or properties. In certain embodiments, a target binding sequence/target sequence comprises 5-50 (e.g., 10-40, 15-30, 15-25, 16-25, 17-25, 18-25, 19-25, 20-25, 10, 11, 12, 13, 14, 15, 16, 17, 18, 19, 20, 21, 22, 23, 24, 25, or more) bases. In certain embodiments, a small number of differences/mismatches is tolerated between (a relevant portion of) an oligonucleotide and its target sequence, including but not limited to the 5′ and/or 3′-end regions of the target and/or oligonucleotide sequence. In certain embodiments, a target sequence is present within a target gene. In certain embodiments, a target sequence is present within a transcript (e.g., an mRNA and/or a pre-mRNA) produced from a target gene.

In certain embodiments, a target sequence includes one or more allelic sites (i.e., positions within a target gene at which allelic variation occurs). In certain embodiments, an allelic site is a mutation. In certain embodiments, an allelic site is a SNP. In some such embodiments, a provided oligonucleotide binds to one allele preferentially or specifically relative to one or more other alleles. In certain embodiments, a provided oligonucleotide binds preferentially to a disease-associated allele. For example, in certain embodiments, an oligonucleotide (or a target-binding sequence portion thereof) provided herein has a sequence that is, fully or at least in part, identical to, or an exact complement of a particular allelic version of a target sequence.

In certain embodiments, an oligonucleotide (or a target-binding sequence portion thereof) provided herein has a sequence that is identical to, or an exact complement of a target sequence comprising an allelic site, or an allelic site, of a disease-associated allele. In certain embodiments, an oligonucleotide provided herein has a target binding sequence that is an exact complement of a target sequence comprising an allelic site of a transcript of an allele (in certain embodiments, a disease-associated allele), wherein the allelic site is a mutation. In certain embodiments, an oligonucleotide provided herein has a target binding sequence that is an exact complement of a target sequence comprising an allelic site of a transcript of an allele (in certain embodiments, a disease-associated allele), wherein the allelic site is a SNP. In certain embodiments, a sequence is any sequence disclosed herein.

Unless otherwise noted, all sequences (including, but not limited to base sequences and patterns of chemistry, modification, and/or stereochemistry) are presented in 5′ to 3′ order, with the 5′ terminal nucleotide identified as the “+1” position and the 3′ terminal nucleotide identified either by the number of nucleotides of the full sequence or by “N”, with the penultimate nucleotide identified, e.g., as “N-i”, and so on.

In certain embodiments, the present disclosure provides compositions and methods related to an oligonucleotide which is specific to a target and which has any format, structural element or base sequence of any oligonucleotide disclosed herein.

In certain embodiments, the present disclosure provides compositions and methods related to an oligonucleotide which is specific to a target and which has or comprises the base sequence of any oligonucleotide disclosed herein, or a region of at least 15 contiguous nucleotides of the base sequence of any oligonucleotide disclosed herein, wherein the first nucleotide of the base sequence or the first nucleotide of the at least 15 contiguous nucleotides can be optionally replaced by T or DNA T.

In certain embodiments, the present disclosure provides compositions and methods for RNA interference directed by a RNAi agent (also referred to as a RNAi oligonucleotides). In certain embodiments, oligonucleotides of such compositions can have a format, structural element or base sequence of an oligonucleotide disclosed herein.

In certain embodiments, the present disclosure provides compositions and methods for RNase H-mediated knockdown of a target gene RNA directed by an oligonucleotide (e.g., an antisense oligonucleotide).

Provided oligonucleotides and oligonucleotide compositions can have any format, structural element or base sequence of any oligonucleotide disclosed herein. In certain embodiments, a structural element is a 5′-end structure, 5′-end region, 5′-nucleotide, seed region, post-seed region, 3′-end region, 3′-terminal dinucleotide, 3′-end cap, or any portion of any of these structures, GC content, long GC stretch, and/or any modification, chemistry, stereochemistry, pattern of modification, chemistry or stereochemistry, or a chemical moiety (e.g., including but not limited to, a targeting moiety, a lipid moiety, a GalNAc moiety, a carbohydrate moiety, etc.), any component, or any combination of any of the above.

In certain embodiments, the present disclosure provides compositions and methods of use of an oligonucleotide.

In certain embodiments, the present disclosure provides compositions and methods of use of an oligonucleotide which can direct both RNA interference and RNase H-mediated knockdown of a target gene RNA. In certain embodiments, oligonucleotides of such compositions can have a format, structural element or base sequence of an oligonucleotide disclosed herein.

In certain embodiments, an oligonucleotide directing a particular event or activity participates in the particular event or activity, e.g., a decrease in the expression, level or activity of a target gene or a gene product thereof. In certain embodiments, an oligonucleotide is deemed to “direct” a particular event or activity when presence of the oligonucleotide in a system in which the event or activity can occur correlates with increased detectable incidence, frequency, intensity and/or level of the event or activity.

In certain embodiments, provided ds oligonucleotides, compositions and methods can be delivered to various regions of the central nervous system. For example, but not by way of limitation, in certain embodiments, provided ds oligonucleotides, compositions and methods can be delivered to various regions of the brain. In certain embodiments, provided ds oligonucleotides, compositions and methods can be delivered to the cerebellum. In certain embodiments, provided ds oligonucleotides, compositions and methods can be delivered to the hippocampus. In certain embodiments, provided ds oligonucleotides, compositions and methods can be delivered to the cortex. In certain embodiments, provided ds oligonucleotides, compositions and methods can be delivered to the striatum. In certain embodiments, provided ds oligonucleotides, compositions and methods can be delivered to the spinal cord. In certain embodiments, provided ds oligonucleotides, compositions and methods can be delivered to the brain stem.

In certain embodiments, provided ds oligonucleotides, compositions and methods can be delivered to various regions of the central nervous system without conjugation to a targeting ligand (e.g., peptide, sugar, etc.), without a delivery system (e.g., lipid nanoparticles, etc.), or combinations thereof.

In certain embodiments, a provided oligonucleotide comprises any one or more structural elements of an oligonucleotide as described herein, e.g., a base sequence (or a portion thereof of at least 15 contiguous bases); a pattern of internucleotidic linkages (or a portion thereof of at least 5 contiguous internucleotidic linkage); a pattern of stereochemistry of internucleotidic linkages (or a portion thereof of at least 5 contiguous internucleotidic linkages); a 5′-end structure; a 5′-end region; a first region; a second region; and a 3′-end region (which can be a 3′-terminal dinucleotide and/or a 3′-end cap); and an optional additional chemical moiety; and, in certain embodiments, at least one structural element comprises a chirally controlled chiral center. In certain embodiments, a 3′-terminal dinucleotide can comprise two total nucleotides. In certain embodiments, an oligonucleotide further comprises a chemical moiety selected from, as non-limiting examples, a targeting moiety, a carbohydrate moiety, a GalNAc moiety, a lipid moiety, and any other chemical moiety described herein or known in the art. In certain embodiments, a moiety that binds APGR is a moiety of GalNAc, or a variant, derivative or modified version thereof, as described herein and/or known in the art. In certain embodiments, an oligonucleotide is a RNAi agent. In certain embodiments, a first region is a seed region. In certain embodiments, a second region is a post-seed region.

In certain embodiments, a provided oligonucleotide comprises any one or more structural elements of a RNAi agent as described herein, e.g., a 5′-end structure; a 5′-end region; a seed region; a post-seed region (the region between the seed region and the 3′-end region); and a 3′-end region (which can be a 3′-terminal dinucleotide and/or a 3′-end cap); and an optional additional chemical moiety; and, in certain embodiments, at least one structural element comprises a chirally controlled chiral center. In certain embodiments, a 3′-terminal dinucleotide can comprise two total nucleotides. In certain embodiments, an oligonucleotide further comprises a chemical moiety selected from, as non-limiting examples, a targeting moiety, a carbohydrate moiety, a GalNAc moiety, and a lipid moiety. In certain embodiments, a moiety that binds APGR is any GalNAc, or variant, derivative or modification thereof, as described herein or known in the art.

In certain embodiments, a provided oligonucleotide comprises any one or more structural elements of an oligonucleotide as described herein, e.g., a 5′-end structure, a 5′-end region, a first region, a second region, a 3′-end region, and an optional additional chemical moiety, wherein at least one structural element comprises a chirally controlled chiral center. In certain embodiments, the oligonucleotide comprises a span of at least 5 total nucleotides without 2′-modifications. In certain embodiments, the oligonucleotide further comprises an additional chemical moiety selected from, as non-limiting examples, a targeting moiety, a carbohydrate moiety, a GalNAc moiety, and a lipid moiety. In certain embodiments, a provided oligonucleotide is capable of directing RNA interference. In certain embodiments, a provided oligonucleotide is capable of directing RNase H-mediated knockdown. In certain embodiments, a provided oligonucleotide is capable of directing both RNA interference and RNase H-mediated knockdown. In certain embodiments, a first region is a seed region. In certain embodiments, a second region is a post-seed region.

In certain embodiments, a provided oligonucleotide comprises any one or more structural elements of a RNAi agent, e.g., a 5′-end structure, a 5′-end region, a seed region, a post-seed region, and a 3′-end region and an optional additional chemical moiety, wherein at least one structural element comprises a chirally controlled chiral center; and, in certain embodiments, the oligonucleotide is also capable of directing RNase H-mediated knockdown of a target gene RNA. In certain embodiments, the oligonucleotide comprises a span of at least 5 total 2′-deoxy nucleotides. In certain embodiments, the oligonucleotide further comprises a chemical moiety selected from, as non-limiting examples, a targeting moiety, a carbohydrate moiety, a GalNAc moiety, and a lipid moiety, and any other additional chemical moiety described herein.

In certain embodiments, the present disclosure demonstrates that oligonucleotide properties can be modulated through chemical modifications. In certain embodiments, the present disclosure provides an oligonucleotide composition comprising a first plurality of oligonucleotides which have a common base sequence and comprise one or more internucleotidic linkage, sugar, and/or base modifications. In certain embodiments, the present disclosure provides an oligonucleotide composition capable of directing RNA interference and comprising a first plurality of oligonucleotides which have a common base sequence and comprise one or more internucleotidic linkage, and/or one or more sugar, and/or one or more base modifications. In certain embodiments, an oligonucleotide or oligonucleotide composition is also capable of directing RNase H-mediated knockdown of a target gene RNA. In certain embodiments, the present disclosure demonstrates that oligonucleotide properties, e.g., activities, toxicities, etc., can be modulated through chemical modifications of sugars, nucleobases, and/or internucleotidic linkages. In certain embodiments, the present disclosure provides an oligonucleotide composition comprising a plurality of oligonucleotides which have a common base sequence, and comprise one or more modified internucleotidic linkages (or “non-natural internucleotidic linkages”, linkages that can be utilized in place of a natural phosphate internucleotidic linkage (—OP(O)(OH)O—), which may exist as a salt form (—OP(O)(O⁻)O—) at a physiological pH) found in natural DNA and RNA), one or more modified sugar moieties, and/or one or more natural phosphate linkages. In certain embodiments, provided oligonucleotides may comprise two or more types of modified internucleotidic linkages. In certain embodiments, a provided oligonucleotide comprises a non-negatively charged internucleotidic linkage. In certain embodiments, a non-negatively charged internucleotidic linkage is a neutral internucleotidic linkage. In certain embodiments, a neutral internucleotidic linkage comprises a cyclic guanidine moiety. Such moieties an optionally substituted. In certain embodiments, a provided oligonucleotide comprises a neutral internucleotidic linkage and another internucleotidic linkage which is not a neutral backbone. In certain embodiments, a provided oligonucleotide comprises a neutral internucleotidic linkage and a phosphorothioate internucleotidic linkage. In certain embodiments, provided oligonucleotide compositions comprising a plurality of oligonucleotides are chirally controlled and level of the plurality of oligonucleotides in the composition is controlled or pre-determined, and oligonucleotides of the plurality share a common stereochemistry configuration at one or more chiral internucleotidic linkages. For example, in certain embodiments, oligonucleotides of a plurality share a common stereochemistry configuration at 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50 or more chiral internucleotidic linkages, each of which is independently Rp or Sp; in certain embodiments, oligonucleotides of a plurality share a common stereochemistry configuration at each chiral internucleotidic linkages. In certain embodiments, a chiral internucleotidic linkage where a controlled level of oligonucleotides of a composition share a common stereochemistry configuration (independently in the Rp or Sp configuration) is referred to as a chirally controlled internucleotidic linkage. In certain embodiments, a modified internucleotidic linkage is a non-negatively charged (neutral or cationic) internucleotidic linkage in that at a pH, (e.g., human physiological pH (˜7.4), pH of a delivery site (e.g., an organelle, cell, tissue, organ, organism, etc.), etc.), it largely (e.g., at least 5%, 10%, 20%, 30%, 40%, 50%, 60%, 70%, 80%, 90%, etc.; in certain embodiments, at least 30%; in certain embodiments, at least 40%; in certain embodiments, at least 50%; in certain embodiments, at least 60%; in certain embodiments, at least 70%; in certain embodiments, at least 80%; in certain embodiments, at least 90%; in certain embodiments, at least 99%; etc.;) exists as a neutral or cationic form (as compared to an anionic form (e.g., —O—P(O)(O⁻)—O— (the anionic form of natural phosphate linkage), —O—P(O)(S⁻)—O— (the anionic form of phosphorothioate linkage), etc.)), respectively. In certain embodiments, a modified internucleotidic linkage is a neutral internucleotidic linkage in that at a pH, it largely exists as a neutral form. In certain embodiments, a modified internucleotidic linkage is a cationic internucleotidic linkage in that at a pH, it largely exists as a cationic form. In certain embodiments, a pH is human physiological pH (˜7.4). In certain embodiments, a modified internucleotidic linkage is a neutral internucleotidic linkage in that at pH 7.4 in a water solution, at least 90% of the internucleotidic linkage exists as its neutral form. In certain embodiments, a modified internucleotidic linkage is a neutral internucleotidic linkage in that in a water solution of the oligonucleotide, at least 50%, 60%, 70%, 80%, 90%, 95%, or 99% of the internucleotidic linkage exists in its neutral form. In certain embodiments, the percentage is at least 90%. In certain embodiments, the percentage is at least 95%. In certain embodiments, the percentage is at least 99%. In certain embodiments, a non-negatively charged internucleotidic linkage, e.g., a neutral internucleotidic linkage, when in its neutral form has no moiety with a pKa that is less than 8, 9, 10, 11. 12, 13, or 14. In certain embodiments, pKa of an internucleotidic linkage in the present disclosure can be represented by pKa of CH₃—the internucleotidic linkage-CH₃ (i.e., replacing the two nucleoside units connected by the internucleotidic linkage with two —CH₃ groups). Without wishing to be bound by any particular theory, in at least some cases, a neutral internucleotidic linkage in an oligonucleotide can provide improved properties and/or activities, e.g., improved delivery, improved resistance to exonucleases and endonucleases, improved cellular uptake, improved endosomal escape and/or improved nuclear uptake, etc., compared to a comparable nucleic acid which does not comprises a neutral internucleotidic linkage.

In certain embodiments, a non-negatively charged internucleotidic linkage has the structure of e.g., of formula I-n-1, I-n-2, I-n-3, II, II-a-1, II-a-2, II-b-1, II-b-2, II-c-1, II-c-2, II-d-1, II-d-2, as described in U.S. Pat. Nos. 9,394,333, 9,744,183, 9,605,019, 9,598,458, 9,982,257, U.S. Ser. No. 10/160,969, U.S. Ser. No. 10/479,995, US 2020/0056173, US 2018/0216107, US 2019/0127733, U.S. Ser. No. 10/450,568, US 2019/0077817, US 2019/0249173, US 2019/0375774, WO 2018/223056, WO 2018/223073, WO 2018/223081, WO 2018/237194, WO 2019/032607, WO 2019/055951, WO 2019/075357, WO 2019/200185, WO 2019/217784, and/or WO 2019/032612 etc. In certain embodiments, a non-negatively charged internucleotidic linkage comprises a cyclic guanidine moiety. In certain embodiments, a modified internucleotidic linkage comprising a cyclic guanidine moiety has the structure of

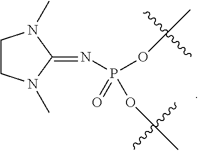 [SMILES: *OP(=O)(N=C1N(C)CCN1C)O*]

In certain embodiments, a modified internucleotidic linkage comprising a cyclic guanidine moiety has the structure of:

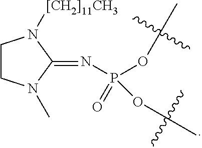

In certain embodiments, a modified internucleotidic linkage comprising a cyclic guanidine moiety has the structure of:

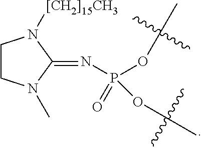

In certain embodiments, a modified internucleotidic linkage comprising a cyclic guanidine moiety has the structure of

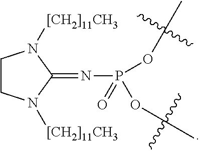

In certain embodiments, a modified internucleotidic linkage comprising a cyclic guanidine moiety has the structure of:

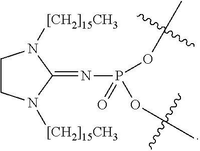

In certain embodiments, a neutral internucleotidic linkage comprising a cyclic guanidine moiety is chirally controlled. In certain embodiments, the present disclosure pertains to a composition comprising an oligonucleotide comprising at least one neutral internucleotidic linkage and at least one phosphorothioate internucleotidic linkage.

In certain embodiments, the present disclosure pertains to a composition comprising an oligonucleotide comprising at least one neutral internucleotidic linkage and at least one phosphorothioate internucleotidic linkage, wherein the phosphorothioate internucleotidic linkage is a chirally controlled internucleotidic linkage in the Sp configuration.

In certain embodiments, the present disclosure pertains to a composition comprising an oligonucleotide comprising at least one neutral internucleotidic linkage and at least one phosphorothioate internucleotidic linkage, wherein the phosphorothioate is a chirally controlled internucleotidic linkage in the Rp configuration.

In certain embodiments, the present disclosure pertains to a composition comprising an oligonucleotide comprising at least one neutral internucleotidic linkage of a neutral internucleotidic linkage comprising a Tmg group

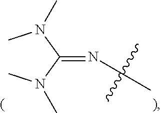 [SMILES: *N=C(N(C)C)N(C)C]

and at least one phosphorothioate.

In certain embodiments, each internucleotidic linkage in an oligonucleotide is independently selected from a natural phosphate linkage, a phosphorothioate linkage, and a non-negatively charged internucleotidic linkage (e.g., n001, n003, n004, n006, n008, n009, n013, n020, n021, n025, n026, n029, n031, n033, n037, n043, n046, n047, n048, n054, n058, or n055). In some embodiments, each internucleotidic linkage in an oligonucleotide is independently selected from a natural phosphate linkage, a phosphorothioate linkage, and a neutral internucleotidic linkage (e.g., n001, n003, n004, n006, n008, n009, n013, n020, n021, n025, n026, n029, n031, n033, n037, n043, n046, n047, n048, n054, n058, or n055).

In certain embodiments, the present disclosure pertains to a composition comprising an oligonucleotide comprising at least one neutral internucleotidic linkage of a neutral internucleotidic linkage comprising a Tmg group, and at least one phosphorothioate, wherein the phosphorothioate is a chirally controlled internucleotidic linkage in the Sp configuration.

In certain embodiments, the present disclosure pertains to a composition comprising an oligonucleotide comprising at least one neutral internucleotidic linkage selected from a neutral internucleotidic linkage of a neutral internucleotidic linkage comprising a Tmg group, and at least one phosphorothioate, wherein the phosphorothioate is a chirally controlled internucleotidic linkage in the Rp configuration.

Various types of internucleotidic linkages differ in properties. Without wishing to be bound by any theory, the present disclosure notes that a natural phosphate linkage (phosphodiester internucleotidic linkage) is anionic and may be unstable when used by itself without other chemical modifications in vivo; a phosphorothioate internucleotidic linkage is anionic, generally more stable in vivo than a natural phosphate linkage, and generally more hydrophobic; a neutral internucleotidic linkage such as one exemplified in the present disclosure comprising a cyclic guanidine moiety is neutral at physiological pH, can be more stable in vivo than a natural phosphate linkage, and more hydrophobic.

In certain embodiments, a chirally controlled neutral internucleotidic linkage sis neutral at physiological pH, chirally controlled, stable in vivo, hydrophobic, and may increase endosomal escape.

In certain embodiments, provided oligonucleotides comprise one or more regions, e.g., a block, wing, core, 5′-end, 3′-end, middle, seed, post-seed region, etc. In certain embodiments, a region (e.g., a block, wing, core, 5′-end, 3′-end, middle region, etc.) comprises a non-negatively charged internucleotidic linkage, e.g., of formula I-n-1, I-n-2, I-n-3, II, II-a-1, II-a-2, II-b-1, II-b-2, II-c-1, II-c-2, II-d-1, II-d-2, etc. as described in U.S. Pat. Nos. 9,394,333, 9,744,183, 9,605,019, 9,598,458, 9,982,257, U.S. Ser. No. 10/160,969, U.S. Ser. No. 10/479,995, US 2020/0056173, US 2018/0216107, US 2019/0127733, U.S. Ser. No. 10/450,568, US 2019/0077817, US 2019/0249173, US 2019/0375774, WO 2018/223056, WO 2018/223073, WO 2018/223081, WO 2018/237194, WO 2019/032607, WO 2019/055951, WO 2019/075357, WO 2019/200185, WO 2019/217784, and/or WO 2019/032612. In certain embodiments, a region comprises a neutral internucleotidic linkage. In certain embodiments, a region comprises an internucleotidic linkage which comprises a cyclic guanidine. In certain embodiments, a region comprises an internucleotidic linkage which comprises a cyclic guanidine moiety. In certain embodiments, a region comprises an internucleotidic linkage having the structure of

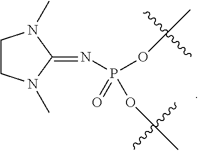 [SMILES: *OP(=O)(N=C1N(C)CCN1C)O*]

In certain embodiments, a modified internucleotidic linkage comprising a cyclic guanidine moiety has the structure of:

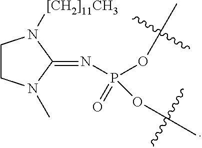

In certain embodiments, a modified internucleotidic linkage comprising a cyclic guanidine moiety has the structure of:

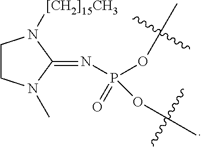

In certain embodiments, a modified internucleotidic linkage comprising a cyclic guanidine moiety has the structure of:

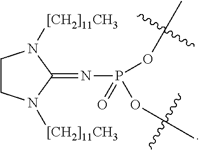

In certain embodiments, a modified internucleotidic linkage comprising a cyclic guanidine moiety has the structure of:

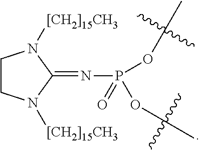

In certain embodiments, such internucleotidic linkages are chirally controlled.
In certain embodiments, a modified internucleotidic linkage has the structure of
In certain embodiments, a modified internucleotidic linkage has the structure of

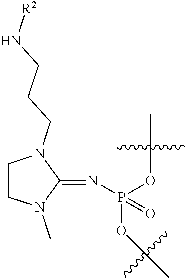

and is optionally chirally controlled,

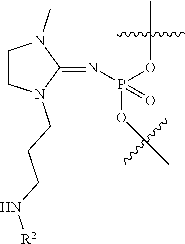

and is optionally chirally controlled or

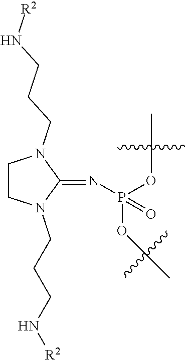

and is optionally chirally controlled wherein R² is H C6-amine

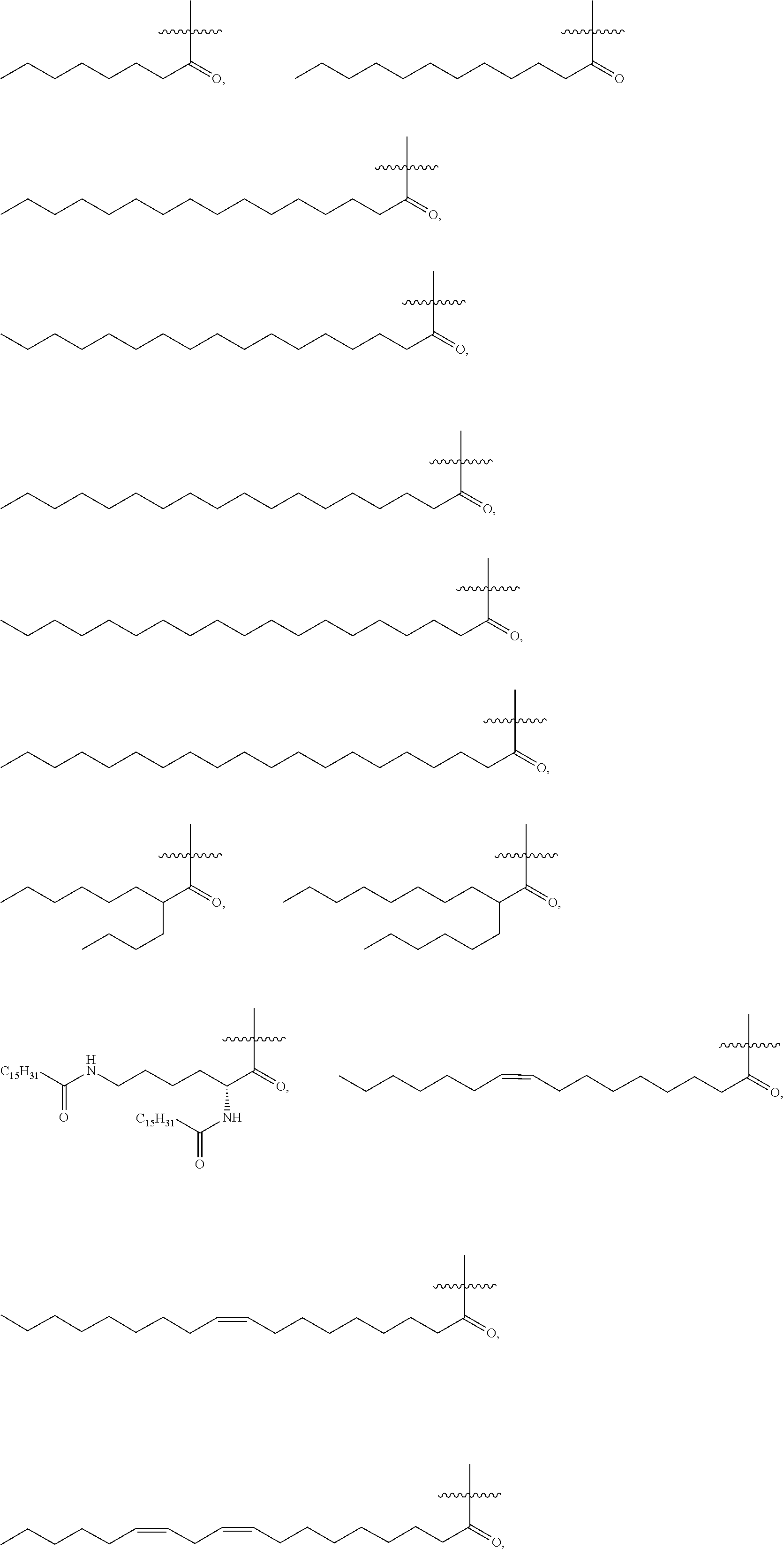

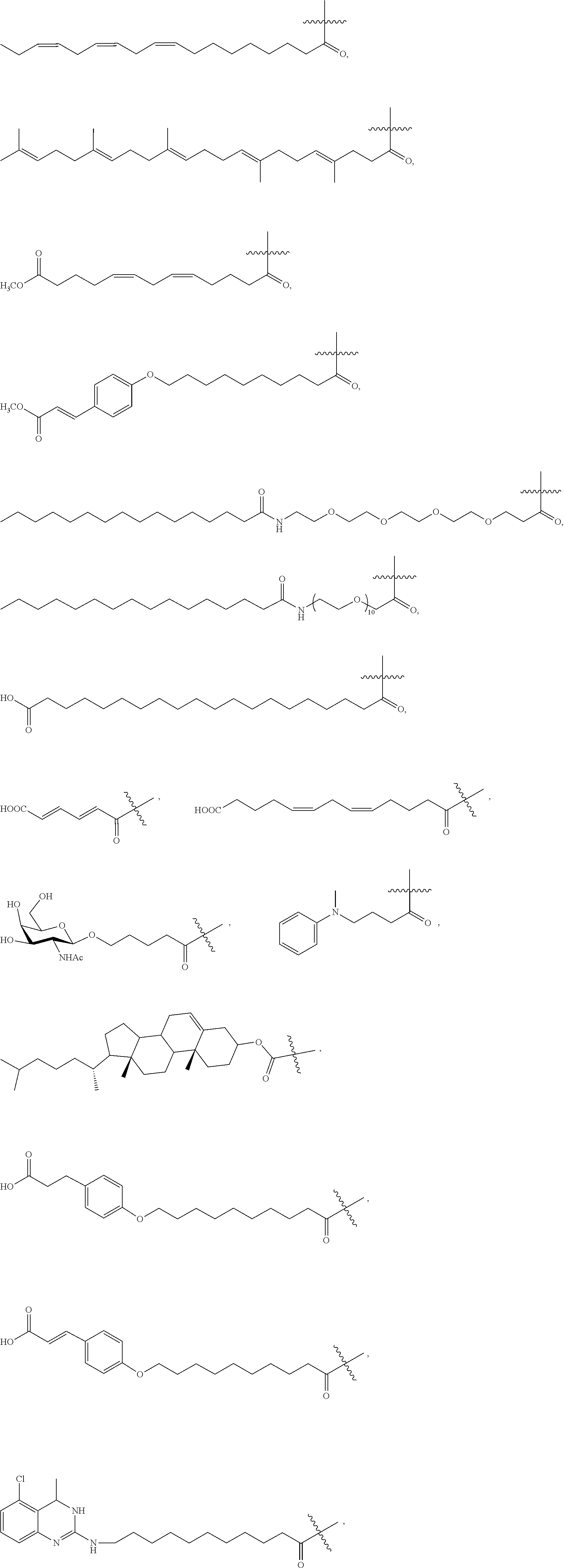

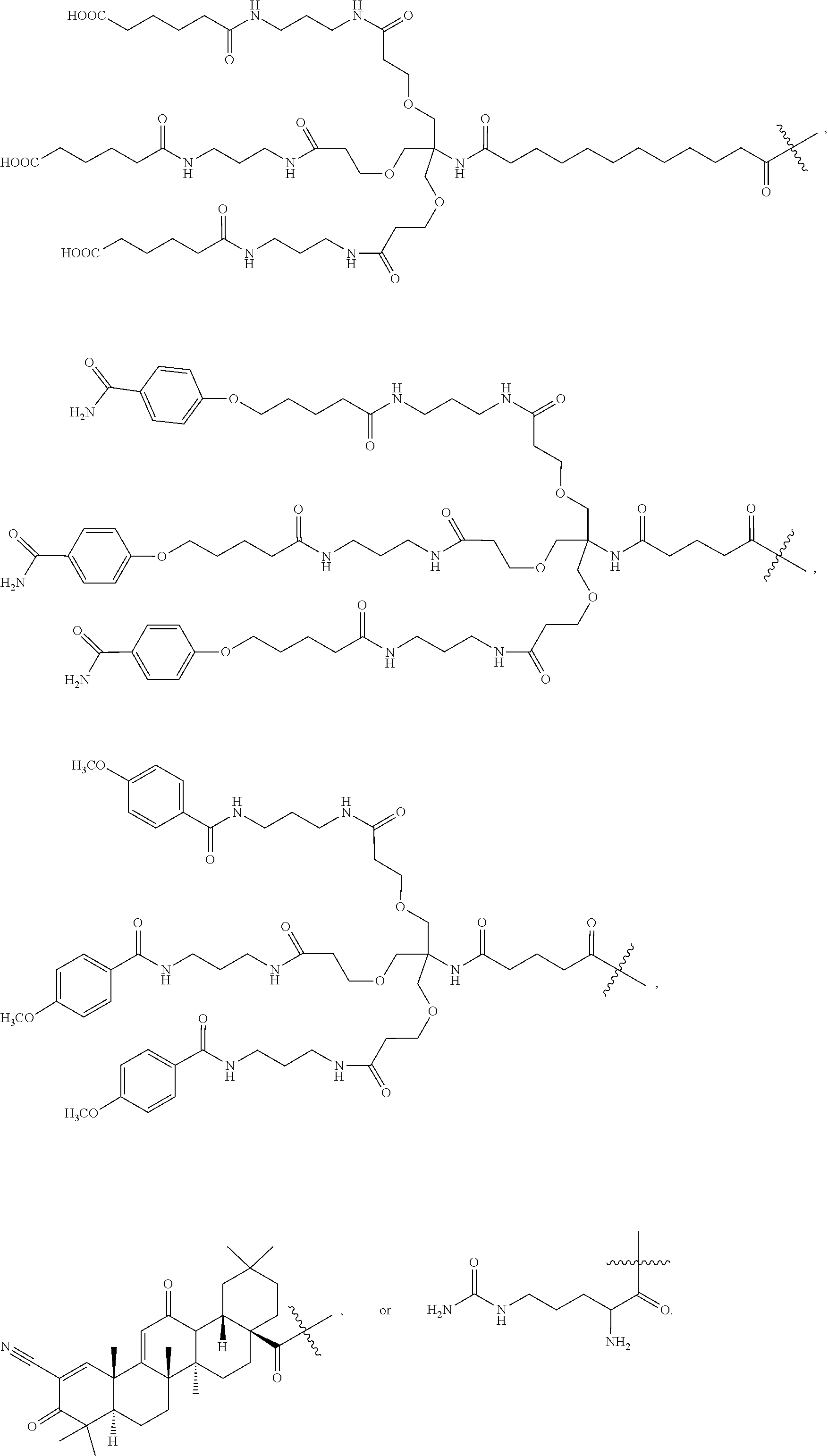

In certain embodiments, a nucleotide is a natural nucleotide. In certain embodiments, a nucleotide is a modified nucleotide. In certain embodiments, a nucleotide is a nucleotide analog. In certain embodiments, a base is a modified base. In certain embodiments, a base is protected nucleobase, such as a protected nucleobase used in oligonucleotide synthesis. In certain embodiments, a base is a base analog. In certain embodiments, a sugar is a modified sugar. In certain embodiments, a sugar is a sugar analog. In certain embodiments, an internucleotidic linkage is a modified internucleotidic linkage. In certain embodiments, a nucleotide comprises a base, a sugar, and an internucleotidic linkage, wherein each of the base, the sugar, and the internucleotidic linkage is independently and optionally naturally-occurring or non-naturally occurring. In certain embodiments, a nucleoside comprises a base and a sugar, wherein each of the base and the sugar is independently and optionally naturally-occurring or non-naturally occurring. Non-limiting examples of nucleotides include DNA (2′-deoxy) and RNA (2′-OH) nucleotides; and those which comprise one or more modifications at the base, sugar and/or internucleotidic linkage. Non-limiting examples of sugars include ribose and deoxyribose; ribose and deoxyribose with 2′-modifications, including but not limited to 2′-F, LNA, 2′-OMe, 2′-O—C16 lipid, and 2′-MOE modifications; and ribose and deoxyribose with 5′-modifications, including, but not limited to 5′-alkyl modifications. In certain embodiments, an internucleotidic linkage is a moiety which does not a comprise a phosphorus but serves to link two natural or non-natural sugars.

In certain embodiments, a composition comprises a multimer of two or more of any: oligonucleotides of a first plurality and/or oligonucleotides of a second plurality, wherein the oligonucleotides of the first and second plurality can independently direct knockdown of the same or different targets independently via RNA interference and/or RNase H-mediated knockdown.

In certain embodiments, the present disclosure provides an oligonucleotide composition comprising a first plurality of oligonucleotides which share:
1) a common base sequence;
2) a common pattern of backbone linkages;
3) common stereochemistry independently at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, or 50 chiral internucleotidic linkages (“chirally controlled internucleotidic linkages”); which composition is chirally controlled in that level of the first plurality of oligonucleotides in the composition is predetermined.

In certain embodiments, an oligonucleotide composition comprising a plurality of oligonucleotides (e.g., a first plurality of oligonucleotides) is chirally controlled in that oligonucleotides of the plurality share a common stereochemistry independently at one or more chiral internucleotidic linkages. In certain embodiments, oligonucleotides of the plurality share a common stereochemistry configuration at 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50 or more chiral internucleotidic linkages, each of which is independently Rp or Sp In certain embodiments, oligonucleotides of the plurality share a common stereochemistry configuration at each chiral internucleotidic linkages. In certain embodiments, a chiral internucleotidic linkage where a predetermined level of oligonucleotides of a composition share a common stereochemistry configuration (independently Rp or Sp) is referred to as a chirally controlled internucleotidic linkage.

In certain embodiments, a predetermined level of oligonucleotides of a provided composition, e.g., a first plurality of oligonucleotides of certain example compositions, comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50 or more chirally controlled internucleotidic linkages.

In certain embodiments, at least 5 internucleotidic linkages are chirally controlled; in certain embodiments, at least 10 internucleotidic linkages are chirally controlled; in certain embodiments, at least 15 internucleotidic linkages are chirally controlled; in certain embodiments, each chiral internucleotidic linkage is chirally controlled.

In certain embodiments, 1%-100% of chiral internucleotidic linkages are chirally controlled. In certain embodiments, at least 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% of chiral internucleotidic linkages are chirally controlled.

In certain embodiments, the present disclosure provides an oligonucleotide composition comprising a first plurality of oligonucleotides which share:
1) a common base sequence;
2) a common pattern of backbone linkages; and
3) a common pattern of backbone chiral centers, which composition is a substantially pure preparation of oligonucleotide in that a predetermined level of the oligonucleotides in the composition have the common base sequence and length, the common pattern of backbone linkages, and the common pattern of backbone chiral centers. In certain embodiments, the common pattern of backbone chiral centers comprises at least one internucleotidic linkage comprising a chirally controlled chiral center. In certain embodiments, a predetermined level of oligonucleotides is at least 1%, 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% of all oligonucleotides in a provided composition. In certain embodiments, a predetermined level of oligonucleotides is at least 1%, 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% of all oligonucleotides in a provided composition that are of or comprise a common base sequence. In certain embodiments, all oligonucleotides in a provided composition that are of or comprise a common base sequence are at least 1%, 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% of all oligonucleotides in the composition. In certain embodiments, a predetermined level of oligonucleotides is at least 1%, 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% of all oligonucleotides in a provided composition that are of or comprise a common base sequence, base modification, sugar modification and/or modified internucleotidic linkage. In certain embodiments, all oligonucleotides in a provided composition that are of or comprise a common base sequence, base modification, sugar modification and/or modified internucleotidic linkage are at least 1%, 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% of all oligonucleotides in the composition. In certain embodiments, a predetermined level of oligonucleotides is at least 1%, 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% of all oligonucleotides in a provided composition that are of or comprise a common base sequence, pattern of base modification, pattern of sugar modification, and/or pattern of modified internucleotidic linkage. In certain embodiments, all oligonucleotides in a provided composition that are of or comprise a common base sequence, pattern of base modification, pattern of sugar modification, and/or pattern of modified internucleotidic linkage are at least 1%, 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% of all oligonucleotides in the composition. In certain embodiments, a predetermined level of oligonucleotides is at least 1%, 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% of all oligonucleotides in a provided composition that share a common base sequence, a common pattern of base modification, a common pattern of sugar modification, and/or a common pattern of modified internucleotidic linkages. In certain embodiments, all oligonucleotides in a provided composition that share a common base sequence, a common pattern of base modification, a common pattern of sugar modification, and/or a common pattern of modified internucleotidic linkages are at least 1%, 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% of all oligonucleotides in the composition. In certain embodiments, a predetermined level is 1-100%. In certain embodiments, a predetermined level is at least 1%. In certain embodiments, a predetermined level is at least 5%. In certain embodiments, a predetermined level is at least 10%. In certain embodiments, a predetermined level is at least 20%. In certain embodiments, a predetermined level is at least 30%. In certain embodiments, a predetermined level is at least 40%. In certain embodiments, a predetermined level is at least 50%. In certain embodiments, a predetermined level is at least 60%. In certain embodiments, a predetermined level is at least 10%. In certain embodiments, a predetermined level is at least 70%. In certain embodiments, a predetermined level is at least 80%. In certain embodiments, a predetermined level is at least 90%. In certain embodiments, a predetermined level is at least 5*(½g), wherein g is the number of chirally controlled internucleotidic linkages. In certain embodiments, a predetermined level is at least 10*(½g), wherein g is the number of chirally controlled internucleotidic linkages. In certain embodiments, a predetermined level is at least 100*(½g), wherein g is the number of chirally controlled internucleotidic linkages. In certain embodiments, a predetermined level is at least (0.80)g, wherein g is the number of chirally controlled internucleotidic linkages. In certain embodiments, a predetermined level is at least (0.80)g, wherein g is the number of chirally controlled internucleotidic linkages. In certain embodiments, a predetermined level is at least (0.80)g, wherein g is the number of chirally controlled internucleotidic linkages. In certain embodiments, a predetermined level is at least (0.85)g, wherein g is the number of chirally controlled internucleotidic linkages. In certain embodiments, a predetermined level is at least (0.90)g, wherein g is the number of chirally controlled internucleotidic linkages. In certain embodiments, a predetermined level is at least (0.95)g, wherein g is the number of chirally controlled internucleotidic linkages. In certain embodiments, a predetermined level is at least (0.96)g, wherein g is the number of chirally controlled internucleotidic linkages. In certain embodiments, a predetermined level is at least (0.97)g, wherein g is the number of chirally controlled internucleotidic linkages. In certain embodiments, a predetermined level is at least (0.98)g, wherein g is the number of chirally controlled internucleotidic linkages. In certain embodiments, a predetermined level is at least (0.99)g, wherein g is the number of chirally controlled internucleotidic linkages. In certain embodiments, to determine level of oligonucleotides having g chirally controlled internucleotidic linkages in a composition, product of diastereopurity of each of the g chirally controlled internucleotidic linkages: (diastereopurity of chirally controlled internucleotidic linkage 1)*(diastereopurity of chirally controlled internucleotidic linkage 2)*. . .*(diastereopurity of chirally controlled internucleotidic linkage g) is utilized as the level, wherein diastereopurity of each chirally controlled internucleotidic linkage is independently represented by diastereopurity of a dimer comprising the same internucleotidic linkage and nucleosides flanking the internucleotidic linkage and prepared under comparable methods as the oligonucleotides (e.g., comparable or preferably identical oligonucleotide preparation cycles, including comparable or preferably identical reagents and reaction conditions). In certain embodiments, levels of oligonucleotides and/or diastereopurity can be determined by analytical methods, e.g., chromatographic, spectrometric, spectroscopic methods or any combinations thereof. Among other things, the present disclosure encompasses the recognition that stereorandom oligonucleotide preparations contain a plurality of distinct chemical entities that differ from one another, e.g., in the stereochemical structure (or stereochemistry) of individual backbone chiral centers within the oligonucleotide chain. Without control of stereochemistry of backbone chiral centers, stereorandom oligonucleotide preparations provide uncontrolled compositions comprising undetermined levels of oligonucleotide stereoisomers. Even though these stereoisomers may have the same base sequence and/or chemical modifications, they are different chemical entities at least due to their different backbone stereochemistry, and they can have, as demonstrated herein, different properties, e.g., sensitivity to nucleases, activities, distribution, etc. In certain embodiments, a particular stereoisomer may be defined, for example, by its base sequence, its length, its pattern of backbone linkages, and its pattern of backbone chiral centers. In certain embodiments, the present disclosure demonstrates that improvements in properties and activities achieved through control of stereochemistry within an oligonucleotide can be comparable to, or even better than those achieved through use of chemical modification.

Among other things, the present disclosure encompasses the recognition that stereorandom oligonucleotide preparations contain a plurality of distinct chemical entities that differ from one another, e.g., in the stereochemical structure (or stereochemistry) of individual backbone chiral centers within the oligonucleotide chain. Without control of stereochemistry of backbone chiral centers, stereorandom oligonucleotide preparations provide uncontrolled compositions comprising undetermined levels of oligonucleotide stereoisomers. Even though these stereoisomers may have the same base sequence and/or chemical modifications, they are different chemical entities at least due to their different backbone stereochemistry, and they can have, as demonstrated herein, different properties, e.g., sensitivity to nucleases, activities, distribution, etc. In certain embodiments, a particular stereoisomer may be defined, for example, by its base sequence, its length, its pattern of backbone linkages, and its pattern of backbone chiral centers. In certain embodiments, the present disclosure demonstrates that improvements in properties and activities achieved through control of stereochemistry within an oligonucleotide can be comparable to, or even better than those achieved through use of chemical modification.

### I. DETAILED DESCRIPTION OF CERTAIN EMBODIMENTS

Technologies of the present disclosure may be understood more readily by reference to the following detailed description of certain embodiments.

### Definitions

As used herein, the following definitions shall apply unless otherwise indicated. For purposes of this disclosure, the elements are identified in accordance with the Periodic Table of the Elements, CAS version, Handbook of Chemistry and Physics, 75th Ed. Additionally, general principles of organic chemistry are described in “Organic Chemistry”, Thomas Sorrell, University Science Books, Sausalito: 1999, and “March's Advanced Organic Chemistry”, 5th Ed., Ed.: Smith, M. B. and March, J., John Wiley & Sons, New York: 2001.

As used herein in the present disclosure, unless otherwise clear from context, (i) the term “a” or “an” may be understood to mean “at least one”; (ii) the term “or” may be understood to mean “and/or”; (iii) the terms “comprising”, “comprise”, “including” (whether used with “not limited to” or not), and “include” (whether used with “not limited to” or not) may be understood to encompass itemized components or steps whether presented by themselves or together with one or more additional components or steps; (iv) the term “another” may be understood to mean at least an additional/second one or more; (v) the terms “about” and “approximately” may be understood to permit standard variation as would be understood by those of ordinary skill in the art; and (vi) where ranges are provided, endpoints are included.

Unless otherwise specified, description of oligonucleotides and elements thereof (e.g., base sequence, sugar modifications, internucleotidic linkages, linkage phosphorus stereochemistry, patterns thereof, etc.) is from 5′ to 3′, with the 5′ terminal nucleotide identified as the “+1” position and the 3′ terminal nucleotide identified either by the number of nucleotides of the full sequence or by “N”, with the penultimate nucleotide identified, e.g., as “N-1”, and so on. As those skilled in the art will appreciate, in certain embodiments, oligonucleotides may be provided and/or utilized as salt forms, particularly pharmaceutically acceptable salt forms, e.g., sodium salts. As those skilled in the art will also appreciate, in certain embodiments, individual oligonucleotides within a composition may be considered to be of the same constitution and/or structure even though, within such composition (e.g., a liquid composition), particular such oligonucleotides might be in different salt form(s) (and may be dissolved and the oligonucleotide chain may exist as an anion form when, e.g., in a liquid composition) at a particular moment in time. For example, those skilled in the art will appreciate that, at a given pH, individual internucleotidic linkages along an oligonucleotide chain may be in an acid (H) form, or in one of a plurality of possible salt forms (e.g., a sodium salt, or a salt of a different cation, depending on which ions might be present in the preparation or composition), and will understand that, so long as their acid forms (e.g., replacing all cations, if any, with H+) are of the same constitution and/or structure, such individual oligonucleotides may properly be considered to be of the same constitution and/or structure.

Aliphatic: As used herein, “aliphatic” means a straight-chain (i.e., unbranched) or branched, substituted or unsubstituted hydrocarbon chain that is completely saturated or that contains one or more units of unsaturation (but not aromatic), or a substituted or unsubstituted monocyclic, bicyclic, or polycyclic hydrocarbon ring that is completely saturated or that contains one or more units of unsaturation (but not aromatic), or combinations thereof. In certain embodiments, aliphatic groups contain 1-50 aliphatic carbon atoms. In certain embodiments, aliphatic groups contain 1-20 aliphatic carbon atoms. In other embodiments, aliphatic groups contain 1-10 aliphatic carbon atoms. In other embodiments, aliphatic groups contain 1-9 aliphatic carbon atoms. In other embodiments, aliphatic groups contain 1-8 aliphatic carbon atoms. In other embodiments, aliphatic groups contain 1-7 aliphatic carbon atoms. In other embodiments, aliphatic groups contain 1-6 aliphatic carbon atoms. In still other embodiments, aliphatic groups contain 1-5 aliphatic carbon atoms, and in yet other embodiments, aliphatic groups contain 1, 2, 3, or 4 aliphatic carbon atoms. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

Alkenyl: As used herein, the term “alkenyl” refers to an aliphatic group, as defined herein, having one or more double bonds.

Alkyl: As used herein, the term “alkyl” is given its ordinary meaning in the art and may include saturated aliphatic groups, including straight-chain alkyl groups, branched-chain alkyl groups, cycloalkyl (alicyclic) groups, alkyl substituted cycloalkyl groups, and cycloalkyl substituted alkyl groups. In certain embodiments, alkyl has 1-100 carbon atoms. In certain embodiments, a straight chain or branched chain alkyl has about 1-20 carbon atoms in its backbone (e.g., C₁-C₂₀ for straight chain, C₂-C₂₀ for branched chain), and alternatively, about 1-10. In certain embodiments, cycloalkyl rings have from about 3-10 carbon atoms in their ring structure where such rings are monocyclic, bicyclic, or polycyclic, and alternatively about 5, 6 or 7 carbons in the ring structure. In certain embodiments, an alkyl group may be a lower alkyl group, wherein a lower alkyl group comprises 1-4 carbon atoms (e.g., C₁-C₄ for straight chain lower alkyls).

Alkynyl: As used herein, the term “alkynyl” refers to an aliphatic group, as defined herein, having one or more triple bonds.

Analog: The term “analog” includes any chemical moiety which differs structurally from a reference chemical moiety or class of moieties, but which is capable of performing at least one function of such a reference chemical moiety or class of moieties. As non-limiting examples, a nucleotide analog differs structurally from a nucleotide but performs at least one function of a nucleotide; a nucleobase analog differs structurally from a nucleobase but performs at least one function of a nucleobase; etc.

Animal: As used herein, the term “animal” refers to any member of the animal kingdom. In certain embodiments, “animal” refers to humans, at any stage of development. In certain embodiments, “animal” refers to non-human animals, at any stage of development. In certain embodiments, the non-human animal is a mammal (e.g., a rodent, a mouse, a rat, a rabbit, a monkey, a dog, a cat, a sheep, cattle, a primate and/or a pig). In certain embodiments, animals include, but are not limited to, mammals, birds, reptiles, amphibians, fish and/or worms. In certain embodiments, an animal may be a transgenic animal, a genetically-engineered animal and/or a clone.

Aryl: The term “aryl”, as used herein, used alone or as part of a larger moiety as in “aralkyl,” “aralkoxy,” or “aryloxyalkyl,” refers to monocyclic, bicyclic or polycyclic ring systems having a total of five to thirty ring members, wherein at least one ring in the system is aromatic. In certain embodiments, an aryl group is a monocyclic, bicyclic or polycyclic ring system having a total of five to fourteen ring members, wherein at least one ring in the system is aromatic, and wherein each ring in the system contains 3 to 7 ring members. In certain embodiments, each monocyclic ring unit is aromatic. In certain embodiments, an aryl group is a biaryl group. The term “aryl” may be used interchangeably with the term “aryl ring.” In certain embodiments of the present disclosure, “aryl” refers to an aromatic ring system which includes, but is not limited to, phenyl, biphenyl, naphthyl, binaphthyl, anthracyl and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like.

Chiral control: As used herein, “chiral control” refers to control of the stereochemical designation of the chiral linkage phosphorus in a chiral internucleotidic linkage within an oligonucleotide. As used herein, a chiral internucleotidic linkage is an internucleotidic linkage whose linkage phosphorus is chiral. In certain embodiments, a control is achieved through a chiral element that is absent from the sugar and base moieties of an oligonucleotide, for example, in certain embodiments, a control is achieved through use of one or more chiral auxiliaries during oligonucleotide preparation, which chiral auxiliaries often are part of chiral phosphoramidites used during oligonucleotide preparation. In contrast to chiral control, a person having ordinary skill in the art will appreciate that conventional oligonucleotide synthesis which does not use chiral auxiliaries cannot control stereochemistry at a chiral internucleotidic linkage if such conventional oligonucleotide synthesis is used to form the chiral internucleotidic linkage. In certain embodiments, the stereochemical designation of each chiral linkage phosphorus in each chiral internucleotidic linkage within an oligonucleotide is controlled.

Chirally controlled oligonucleotide composition: The terms “chirally controlled oligonucleotide composition”, “chirally controlled nucleic acid composition”, and the like, as used herein, refers to a composition that comprises a plurality of oligonucleotides (or nucleic acids) which share a common base sequence, wherein the plurality of oligonucleotides (or nucleic acids) share the same linkage phosphorus stereochemistry at one or more chiral internucleotidic linkages (chirally controlled or stereodefined internucleotidic linkages, whose chiral linkage phosphorus is Rp or Sp in the composition (“stereodefined”), not a random Rp and Sp mixture as non-chirally controlled internucleotidic linkages). In certain embodiments, a chirally controlled oligonucleotide composition comprises a plurality of oligonucleotides (or nucleic acids) that share: 1) a common base sequence, 2) a common pattern of backbone linkages, and 3) a common pattern of backbone phosphorus modifications, wherein the plurality of oligonucleotides (or nucleic acids) share the same linkage phosphorus stereochemistry at one or more chiral internucleotidic linkages (chirally controlled or stereodefined internucleotidic linkages, whose chiral linkage phosphorus is Rp or Sp in the composition (“stereodefined”), not a random Rp and Sp mixture as non-chirally controlled internucleotidic linkages). Level of the plurality of oligonucleotides (or nucleic acids) in a chirally controlled oligonucleotide composition is pre-determined/controlled or enriched (e.g., through chirally controlled oligonucleotide preparation to stereoselectively form one or more chiral internucleotidic linkages) compared to a random level in a non-chirally controlled oligonucleotide composition. In certain embodiments, about 1%-100%, (e.g., about 5%-1⁰⁰%, 10%-10⁰%, 20%-100%, 30%-100%, 40%-100%, 50%-100%, 60%-100%, 70%-100%, 80-100%, 90-100%, 95-100%, 50%-90%, or about 5%, 10%, 20%, 30%, 40%, 50%, 60%, 70%, 80%, 85%, 90%, 91%, 92%, 93%, 94%, 95%, 96%, 97%, 98%, 99%, or 100%, or at least 5%, 10%, 20%, 30%, 40%, 50%, 60%, 70%, 80%, 85%, 90%, 91%, 92%, 93%, 94%, 95%, 96%, 97%, 98%, or 99%) of all oligonucleotides in a chirally controlled oligonucleotide composition are oligonucleotides of the plurality. In certain embodiments, about 1%-100%, (e.g., about 5%-100%, 10%-100%, 20%-100%, 30%-100%, 40%-100%, 50%-100%, 60%-100%, 70%-100%, 80-100%, 90-100%, 95-100%, 50%-90%, or about 5%, 10%, 20%, 30%, 40%, 50%, 60%, 70%, 80%, 85%, 90%, 91%, 92%, 93%, 94%, 95%, 96%, 97%, 98%, 99%, or 100%, or at least 5%, 10%, 20%, 30%, 40%, 50%, 60%, 70%, 80%, 85%, 90%, 91%, 92%, 93%, 94%, 95%, 96%, 97%, 98%, or 99%) of all oligonucleotides in a chirally controlled oligonucleotide composition that share the common base sequence, the common pattern of backbone linkages, and the common pattern of backbone phosphorus modifications are oligonucleotides of the plurality. In certain embodiments, a level is about 1%-100%, (e.g., about 5%-100%, 10%-100%, 20%-100%, 30%-100%, 40%-100%, 50%-100%, 60%-100%, 70%-100%, 80-100%, 90-100%, 95-100%, 50%-90%, or about 5%, 10%, 20%, 30%, 40%, 50%, 60%, 70%, 80%, 85%, 90%, 91%, 92%, 93%, 94%, 95%, 96%, 97%, 98%, 99%, or 100%, or at least 5%, 10%, 20%, 30%, 40%, 50%, 60%, 70%, 80%, 85%, 90%, 91%, 92%, 93%, 94%, 95%, 96%, 97%, 98%, or 99%) of all oligonucleotides in a composition, or of all oligonucleotides in a composition that share a common base sequence (e.g., of a plurality of oligonucleotide or an oligonucleotide type), or of all oligonucleotides in a composition that share a common base sequence, a common pattern of backbone linkages, and a common pattern of backbone phosphorus modifications, or of all oligonucleotides in a composition that share a common base sequence, a common patter of base modifications, a common pattern of sugar modifications, a common pattern of internucleotidic linkage types, and/or a common pattern of internucleotidic linkage modifications. In certain embodiments, the plurality of oligonucleotides share the same stereochemistry at about 1-50 (e.g., about 1-10, 1-20, 5-10, 5-20, 10-15, 10-20, 10-25, 10-30, or about 1, 2, 3, 4, 5, 6, 7, 8, 9, 10, 11, 12, 13, 14, 15, 16, 17, 18, 19, or 20, or at least 1, 2, 3, 4, 5, 6, 7, 8, 9, 10, 11, 12, 13, 14, 15, 16, 17, 18, 19, or 20) chiral internucleotidic linkages. In certain embodiments, the plurality of oligonucleotides share the same stereochemistry at about 1%-100% (e.g., about 5%-100%, 10%-100%, 20%-100^{%}, 30%-100%, 40%-100%, 50%-100%, 60%-100%, 70%-100%, 80-100%, 90-100%, 95-100%, 50^{%}-90%, about 5%, 10%, 15%, 20%, 25%, 30%, 35%, 40%, 45%, 50%, 55%, 60%, 65%, 70%, 75%, 80%, 85%, 90%, 95%, or 100%, or at least 5%, 10%, 15%, 20%, 25%, 30%, 35%, 40%, 45%, 50%, 55%, 60%, 65%, 70%, 75%, 80%, 85%, 90%, 95%, or 99%) of chiral internucleotidic linkages. In certain embodiments, oligonucleotides (or nucleic acids) of a plurality share the same pattern of sugar and/or nucleobase modifications, in any. In certain embodiments, oligonucleotides (or nucleic acids) of a plurality are various forms of the same oligonucleotide (e.g., acid and/or various salts of the same oligonucleotide). In certain embodiments, oligonucleotides (or nucleic acids) of a plurality are of the same constitution. In certain embodiments, level of the oligonucleotides (or nucleic acids) of the plurality is about 1%-100%, (e.g., about 5%-100%, 10%-100%, 20%-100%, 30%-100%, 40%-100%, 50%-100%, 60%-100%, 70%-100%, 80-100%, 90-100%, 95-100%, 50%-90%, or about 5%, 10%, 20%, 30%, 40%, 50%, 60%, 70%, 80%, 85%, 90%, 91%, 92%, 93%, 94%, 95%, 96%, 97%, 98%, 99%, or 100%, or at least 5%, 10%, 20%, 30%, 40%, 50%, 60%, 70%, 80%, 85%, 90%, 91%, 92%, 93%, 94%, 95%, 96%, 97%, 98%, or 99%) of all oligonucleotides (or nucleic acids) in a composition that share the same constitution as the oligonucleotides (or nucleic acids) of the plurality. In certain embodiments, each chiral internucleotidic linkage is a chiral controlled internucleotidic linkage, and the composition is a completely chirally controlled oligonucleotide composition. In certain embodiments, oligonucleotides (or nucleic acids) of a plurality are structurally identical. In certain embodiments, a chirally controlled internucleotidic linkage has a diastereopurity of at least 80%, 85%, 90%, 91%, 92%, 93%, 94%, 95%, 96%, 97%, 98%, 99% or 99.5%, typically at least 90%, 91%, 92%, 93%, 94%, 95%, 96%, 97%, 98%, 99% or 99.5%. In certain embodiments, a chirally controlled internucleotidic linkage has a diastereopurity of at least 95%. In certain embodiments, a chirally controlled internucleotidic linkage has a diastereopurity of at least 96%. In certain embodiments, a chirally controlled internucleotidic linkage has a diastereopurity of at least 97%. In certain embodiments, a chirally controlled internucleotidic linkage has a diastereopurity of at least 98%. In certain embodiments, a chirally controlled internucleotidic linkage has a diastereopurity of at least 99%. In certain embodiments, a percentage of a level is or is at least (DS)^{nc}, wherein DS is a diastereopurity as described in the present disclosure (e.g., 90%, 91%, 92%, 93%, 94%, 95%, 96%, 97%, 98%, 99% or 99.5% or more) and nc is the number of chirally controlled internucleotidic linkages as described in the present disclosure (e.g., 1-50, 1-40, 1-30, 1-25, 1-20, 5-50, 5-40, 5-30, 5-25, 5-20, 1, 2, 3, 4, 5, 6, 7, 8, 9, 10, 11, 12, 13, 14, 15, 16, 17, 18, 19, 20, 21, 22, 23, 24, 25 or more). In certain embodiments, a percentage of a level is or is at least (DS)^{nc}, wherein DS is 95%-100%. For example, when DS is 99% and nc is 10, the percentage is or is at least 90% ((99%)¹⁰≈0.90=90%). In certain embodiments, level of a plurality of oligonucleotides in a composition is represented as the product of the diastereopurity of each chirally controlled internucleotidic linkage in the oligonucleotides. In certain embodiments, diastereopurity of an internucleotidic linkage connecting two nucleosides in an oligonucleotide (or nucleic acid) is represented by the diastereopurity of an internucleotidic linkage of a dimer connecting the same two nucleosides, wherein the dimer is prepared using comparable conditions, in some instances, identical synthetic cycle conditions (e.g., for the linkage between Nx and Ny in an oligonucleotide . . . NxNy . . . , the dimer is NxNy). In certain embodiments, not all chiral internucleotidic linkages are chiral controlled internucleotidic linkages, and the composition is a partially chirally controlled oligonucleotide composition. In certain embodiments, a non-chirally controlled internucleotidic linkage has a diastereopurity of less than about 80%, 75%, 70%, 65%, 60%, 55%, or of about 50%, as typically observed in stereorandom oligonucleotide compositions (e.g., as appreciated by those skilled in the art, from traditional oligonucleotide synthesis, e.g., the phosphoramidite method). In certain embodiments, oligonucleotides (or nucleic acids) of a plurality are of the same type. In certain embodiments, a chirally controlled oligonucleotide composition comprises non-random or controlled levels of individual oligonucleotide or nucleic acids types. For instance, in certain embodiments a chirally controlled oligonucleotide composition comprises one and no more than one oligonucleotide type. In certain embodiments, a chirally controlled oligonucleotide composition comprises more than one oligonucleotide type. In certain embodiments, a chirally controlled oligonucleotide composition comprises multiple oligonucleotide types. In certain embodiments, a chirally controlled oligonucleotide composition is a composition of oligonucleotides of an oligonucleotide type, which composition comprises a non-random or controlled level of a plurality of oligonucleotides of the oligonucleotide type.

Comparable: The term “comparable” is used herein to describe two (or more) sets of conditions or circumstances that are sufficiently similar to one another to permit comparison of results obtained or phenomena observed. In certain embodiments, comparable sets of conditions or circumstances are characterized by a plurality of substantially identical features and one or a small number of varied features. Those of ordinary skill in the art will appreciate that sets of conditions are comparable to one another when characterized by a sufficient number and type of substantially identical features to warrant a reasonable conclusion that differences in results obtained or phenomena observed under the different sets of conditions or circumstances are caused by or indicative of the variation in those features that are varied.

Cycloaliphatic: The term “cycloaliphatic,” “carbocycle,” “carbocyclyl,” “carbocyclic radical,” and “carbocyclic ring,” are used interchangeably, and as used herein, refer to saturated or partially unsaturated, but non-aromatic, cyclic aliphatic monocyclic, bicyclic, or polycyclic ring systems, as described herein, having, unless otherwise specified, from 3 to 30 ring members. Cycloaliphatic groups include, without limitation, cyclopropyl, cyclobutyl, cyclopentyl, cyclopentenyl, cyclohexyl, cyclohexenyl, cycloheptyl, cycloheptenyl, cyclooctyl, cyclooctenyl, norbomyl, adamantyl, and cyclooctadienyl. In certain embodiments, a cycloaliphatic group has 3-6 carbons. In certain embodiments, a cycloaliphatic group is saturated and is cycloalkyl. The term “cycloaliphatic” may also include aliphatic rings that are fused to one or more aromatic or nonaromatic rings, such as decahydronaphthyl or tetrahydronaphthyl. In certain embodiments, a cycloaliphatic group is bicyclic. In certain embodiments, a cycloaliphatic group is tricyclic. In certain embodiments, a cycloaliphatic group is polycyclic. In certain embodiments, “cycloaliphatic” refers to C₃-C₆ monocyclic hydrocarbon, or C₈-C₁₀ bicyclic or polycyclic hydrocarbon, that is completely saturated or that contains one or more units of unsaturation, but which is not aromatic, that has a single point of attachment to the rest of the molecule, or a C₉-C₁₆ polycyclic hydrocarbon that is completely saturated or that contains one or more units of unsaturation, but which is not aromatic, that has a single point of attachment to the rest of the molecule.

Heteroaliphatic: The term “heteroaliphatic”, as used herein, is given its ordinary meaning in the art and refers to aliphatic groups as described herein in which one or more carbon atoms are independently replaced with one or more heteroatoms (e.g., oxygen, nitrogen, sulfur, silicon, phosphorus, and the like). In certain embodiments, one or more units selected from C, CH, CH₂, and CH₃ are independently replaced by one or more heteroatoms (including oxidized and/or substituted forms thereof). In certain embodiments, a heteroaliphatic group is heteroalkyl. In certain embodiments, a heteroaliphatic group is heteroalkenyl.

Heteroalkyl: The term “heteroalkyl”, as used herein, is given its ordinary meaning in the art and refers to alkyl groups as described herein in which one or more carbon atoms are independently replaced with one or more heteroatoms (e.g., oxygen, nitrogen, sulfur, silicon, phosphorus, and the like). Examples of heteroalkyl groups include, but are not limited to, alkoxy, poly(ethylene glycol)-, alkyl-substituted amino, tetrahydrofuranyl, piperidinyl, morpholinyl, etc.

Heteroaryl: The terms “heteroaryl” and “heteroar-”, as used herein, used alone or as part of a larger moiety, e.g., “heteroaralkyl,” or “heteroaralkoxy,” refer to monocyclic, bicyclic or polycyclic ring systems having a total of five to thirty ring members, wherein at least one ring in the system is aromatic and at least one aromatic ring atom is a heteroatom. In certain embodiments, a heteroaryl group is a group having 5 to 10 ring atoms (i.e., monocyclic, bicyclic or polycyclic), in certain embodiments 5, 6, 9, or 10 ring atoms. In certain embodiments, each monocyclic ring unit is aromatic. In certain embodiments, a heteroaryl group has 6, 10, or 14 π electrons shared in a cyclic array; and having, in addition to carbon atoms, from one to five heteroatoms. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. In certain embodiments, a heteroaryl is a heterobiaryl group, such as bipyridyl and the like. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring. Non-limiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one. A heteroaryl group may be monocyclic, bicyclic or polycyclic. The term “heteroaryl” may be used interchangeably with the terms “heteroaryl ring,” “heteroaryl group,” or “heteroaromatic,” any of which terms include rings that are optionally substituted. The term “heteroaralkyl” refers to an alkyl group substituted by a heteroaryl group, wherein the alkyl and heteroaryl portions independently are optionally substituted.

Heteroatom: The term “heteroatom”, as used herein, means an atom that is not carbon or hydrogen. In certain embodiments, a heteroatom is boron, oxygen, sulfur, nitrogen, phosphorus, or silicon (including oxidized forms of nitrogen, sulfur, phosphorus, or silicon; charged forms of nitrogen (e.g., quaternized forms, forms as in iminium groups, etc.), phosphorus, sulfur, oxygen; etc.). In certain embodiments, a heteroatom is silicon, phosphorus, oxygen, sulfur or nitrogen. In certain embodiments, a heteroatom is silicon, oxygen, sulfur or nitrogen. In certain embodiments, a heteroatom is oxygen, sulfur or nitrogen.

Heterocycle: As used herein, the terms “heterocycle,” “heterocyclyl,” “heterocyclic radical,” and “heterocyclic ring”, as used herein, are used interchangeably and refer to a monocyclic, bicyclic or polycyclic ring moiety (e.g., 3-30 membered) that is saturated or partially unsaturated and has one or more heteroatom ring atoms. In certain embodiments, a heterocyclyl group is a stable 5- to 7-membered monocyclic or 7- to 10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes substituted nitrogen. As an example, in a saturated or partially unsaturated ring having 0-3 heteroatoms selected from oxygen, sulfur and nitrogen, the nitrogen may be N (as in 3,4-dihydro-2H-pyrrolyl), NH (as in pyrrolidinyl), or ⁺NR (as in N-substituted pyrrolidinyl). A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothienyl, pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle,” “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl. A heterocyclyl group may be monocyclic, bicyclic or polycyclic. The term “heterocyclylalkyl” refers to an alkyl group substituted by a heterocyclyl, wherein the alkyl and heterocyclyl portions independently are optionally substituted.

Identity: As used herein, the term “identity” refers to the overall relatedness between polymeric molecules, e.g., between nucleic acid molecules (e.g., oligonucleotides, DNA, RNA, etc.) and/or between polypeptide molecules. In certain embodiments, polymeric molecules are considered to be “substantially identical” to one another if their sequences are at least 25%, 30%, 35%, 40%, 45%, 50%, 55%, 60%, 65%, 70%, 75%, 80%, 85%, 90%, 95%, or 99% identical. Calculation of the percent identity of two nucleic acid or polypeptide sequences, for example, can be performed by aligning the two sequences for optimal comparison purposes (e.g., gaps can be introduced in one or both of a first and a second sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or substantially 100% of the length of a reference sequence. The nucleotides at corresponding positions are then compared. When a position in the first sequence is occupied by the same residue (e.g., nucleotide or amino acid) as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using the algorithm of Meyers and Miller (CABIOS, 1989, 4: 11-17), which has been incorporated into the ALIGN program (version 2.0). In some exemplary embodiments, nucleic acid sequence comparisons made with the ALIGN program use a PAM120 weight residue table, a gap length penalty of 12 and a gap penalty of 4. The percent identity between two nucleotide sequences can, alternatively, be determined using the GAP program in the GCG software package using an NWSgapdna.CMP matrix.

Internucleotidic linkage: As used herein, the phrase “internucleotidic linkage” refers generally to a linkage linking nucleoside units of an oligonucleotide or a nucleic acid. In certain embodiments, an internucleotidic linkage is a phosphodiester linkage, as extensively found in naturally occurring DNA and RNA molecules (natural phosphate linkage (—OP(═O)(OH)O—), which as appreciated by those skilled in the art may exist as a salt form). In certain embodiments, an internucleotidic linkage is a modified internucleotidic linkage (not a natural phosphate linkage). In certain embodiments, an internucleotidic linkage is a “modified internucleotidic linkage” wherein at least one oxygen atom or —OH of a phosphodiester linkage is replaced by a different organic or inorganic moiety. In certain embodiments, such an organic or inorganic moiety is selected from ═S, ═Se, ═NR′, —SR′, —SeR′, —N(R′)₂, B(R′)₃, —S—, —Se—, and —N(R′)—, wherein each R′ is independently as defined and described in the present disclosure. In certain embodiments, an internucleotidic linkage is a phosphotriester linkage, phosphorothioate linkage (or phosphorothioate diester linkage, —OP(═O)(SH)O—, which as appreciated by those skilled in the art may exist as a salt form), or phosphorothioate triester linkage. In certain embodiments, a modified internucleotidic linkage is a phosphorothioate linkage. In certain embodiments, a modified internucleotidic linkage is a phosphoryl guanidine linkage. In certain embodiments, an internucleotidic linkage is one of, e.g., PNA (peptide nucleic acid) or PMO (phosphorodiamidate Morpholino oligomer) linkage. In certain embodiments, a modified internucleotidic linkage is a non-negatively charged internucleotidic linkage. In certain embodiments, a modified internucleotidic linkage is a neutral internucleotidic linkage (e.g., n001 in certain provided oligonucleotides). In certain a embodiments, a phosphoryl guanidine linkage is a non-negatively charged linkage or a neutral internucleotidic linkage. It is understood by a person of ordinary skill in the art that an internucleotidic linkage may exist as an anion or cation at a given pH due to the existence of acid or base moieties in the linkage. In certain embodiments, a modified internucleotidic linkages is a modified internucleotidic linkages designated as s, s1, s2, s3, s4, s5, s6, s7, s8, s9, s10, s11, s12, s13, s14, s15, s16, s17 and s18 as described in WO 2017/210647.

In vitro: As used herein, the term “in vitro” refers to events that occur in an artificial environment, e.g., in a test tube or reaction vessel, in cell culture, etc., rather than within an organism (e.g., animal, plant and/or microbe).

In vivo: As used herein, the term “in vivo” refers to events that occur within an organism (e.g., animal, plant and/or microbe).

Linkage phosphorus: as defined herein, the phrase “linkage phosphorus” is used to indicate that the particular phosphorus atom being referred to is the phosphorus atom present in the internucleotidic linkage, which phosphorus atom corresponds to the phosphorus atom of a phosphodiester internucleotidic linkage as occurs in naturally occurring DNA and RNA. In certain embodiments, a linkage phosphorus atom is in a modified internucleotidic linkage, wherein each oxygen atom of a phosphodiester linkage is optionally and independently replaced by an organic or inorganic moiety. In certain embodiments, a linkage phosphorus atom is chiral (e.g., as in phosphorothioate internucleotidic linkages). In certain embodiments, a linkage phosphorus atom is achiral (e.g., as in natural phosphate linkages).

Modified nucleobase: The terms “modified nucleobase”, “modified base” and the like refer to a chemical moiety which is chemically distinct from a nucleobase, but which is capable of performing at least one function of a nucleobase. In certain embodiments, a modified nucleobase is a nucleobase which comprises a modification. In certain embodiments, a modified nucleobase is capable of at least one function of a nucleobase, e.g., forming a moiety in a polymer capable of base-pairing to a nucleic acid comprising an at least complementary sequence of bases. In certain embodiments, a modified nucleobase is substituted A, T, C, G, or U, or a substituted tautomer of A, T, C, G, or U. In certain embodiments, a modified nucleobase in the context of oligonucleotides refer to a nucleobase that is not A, T, C, G or U.

Modified nucleoside: The term “modified nucleoside” refers to a moiety derived from or chemically similar to a natural nucleoside, but which comprises a chemical modification which differentiates it from a natural nucleoside. Non-limiting examples of modified nucleosides include those which comprise a modification at the base and/or the sugar. Non-limiting examples of modified nucleosides include those with a 2′ modification at a sugar. Non-limiting examples of modified nucleosides also include abasic nucleosides (which lack a nucleobase). In certain embodiments, a modified nucleoside is capable of at least one function of a nucleoside, e.g., forming a moiety in a polymer capable of base-pairing to a nucleic acid comprising an at least complementary sequence of bases.

Modified nucleotide: The term “modified nucleotide” includes any chemical moiety which differs structurally from a natural nucleotide but is capable of performing at least one function of a natural nucleotide. In certain embodiments, a modified nucleotide comprises a modification at a sugar, base and/or internucleotidic linkage. In certain embodiments, a modified nucleotide comprises a modified sugar, modified nucleobase and/or modified internucleotidic linkage. In certain embodiments, a modified nucleotide is capable of at least one function of a nucleotide, e.g., forming a subunit in a polymer capable of base-pairing to a nucleic acid comprising an at least complementary sequence of bases.

Modified sugar: The term “modified sugar” refers to a moiety that can replace a sugar. A modified sugar mimics the spatial arrangement, electronic properties, or some other physicochemical property of a sugar. In certain embodiments, as described in the present disclosure, a modified sugar is substituted ribose or deoxyribose. In certain embodiments, a modified sugar comprises a 2′-modification. Examples of useful 2′-modification are widely utilized in the art and described herein. In certain embodiments, a 2′-modification is 2′-F. In certain embodiments, a 2′-modification is 2′-OR, wherein R is optionally substituted C₁₋₁₀ aliphatic. In certain embodiments, the 2′ modification is a 2′-O—C16 lipid modification. In certain embodiments, a 2′-modification is 2′-OMe. In certain embodiments, a 2′-modification is 2′-MOE. In certain embodiments, a modified sugar comprises a 5′-modification. In certain embodiments, each modified sugar independently comprises a 5′-modification. In certain embodiments, a 5′-modification is 5′-alkyl modification. In certain embodiments, a 5′-alkyl modification is a 5′-methyl modification. In particular embodiments, a 5′-methyl modification is a 5′-(R)-methyl modification. In certain embodiments, a 5′-methyl modification is a 5′-(S)-methyl modification. In certain embodiments, a modified sugar is a bicyclic sugar (e.g., a sugar used in LNA, BNA, etc.). In certain embodiments, in the context of oligonucleotides, a modified sugar is a sugar that is not ribose or deoxyribose as typically found in natural RNA or DNA.

Nucleic acid: The term “nucleic acid”, as used herein, includes any nucleotides and polymers thereof. The term “polynucleotide”, as used herein, refers to a polymeric form of nucleotides of any length, either ribonucleotides (RNA) or deoxyribonucleotides (DNA) or a combination thereof. These terms refer to the primary structure of the molecules and, thus, include double- and single-stranded DNA, and double- and single-stranded RNA. These terms include, as equivalents, analogs of either RNA or DNA comprising modified nucleotides and/or modified polynucleotides, such as, though not limited to, methylated, protected and/or capped nucleotides or polynucleotides. The terms encompass poly- or oligo-ribonucleotides (RNA) and poly- or oligo-deoxyribonucleotides (DNA); RNA or DNA derived from N-glycosides or C-glycosides of nucleobases and/or modified nucleobases; nucleic acids derived from sugars and/or modified sugars; and nucleic acids derived from phosphate bridges and/or modified internucleotidic linkages. The term encompasses nucleic acids containing any combinations of nucleobases, modified nucleobases, sugars, modified sugars, phosphate bridges or modified internucleotidic linkages. Examples include, and are not limited to, nucleic acids containing ribose moieties, nucleic acids containing deoxy-ribose moieties, nucleic acids containing both ribose and deoxyribose moieties, nucleic acids containing ribose and modified ribose moieties. Unless otherwise specified, the prefix poly-refers to a nucleic acid containing 2 to about 10,000 nucleotide monomer units and wherein the prefix oligo-refers to a nucleic acid containing 2 to about 200 nucleotide monomer units.

Nucleobase: The term “nucleobase” refers to the parts of nucleic acids that are involved in the hydrogen-bonding that binds one nucleic acid strand to another complementary strand in a sequence specific manner. The most common naturally-occurring nucleobases are adenine (A), guanine (G), uracil (U), cytosine (C), and thymine (T). In certain embodiments, a naturally-occurring nucleobases are modified adenine, guanine, uracil, cytosine, or thymine. In certain embodiments, a naturally-occurring nucleobases are methylated adenine, guanine, uracil, cytosine, or thymine. In certain embodiments, a nucleobase comprises a heteroaryl ring wherein a ring atom is nitrogen, and when in a nucleoside, the nitrogen is bonded to a sugar moiety. In certain embodiments, a nucleobase comprises a heterocyclic ring wherein a ring atom is nitrogen, and when in a nucleoside, the nitrogen is bonded to a sugar moiety. In certain embodiments, a nucleobase is a “modified nucleobase,” a nucleobase other than adenine (A), guanine (G), uracil (U), cytosine (C), and thymine (T). In certain embodiments, a modified nucleobase is substituted A, T, C, G or U. In certain embodiments, a modified nucleobase is a substituted tautomer of A, T, C, G, or U. In certain embodiments, a modified nucleobase is methylated adenine, guanine, uracil, cytosine, or thymine. In certain embodiments, a modified nucleobase mimics the spatial arrangement, electronic properties, or some other physicochemical property of the nucleobase and retains the property of hydrogen-bonding that binds one nucleic acid strand to another in a sequence specific manner. In certain embodiments, a modified nucleobase can pair with all of the five naturally occurring bases (uracil, thymine, adenine, cytosine, or guanine) without substantially affecting the melting behavior, recognition by intracellular enzymes or activity of the oligonucleotide duplex. As used herein, the term “nucleobase” also encompasses structural analogs used in lieu of natural or naturally-occurring nucleotides, such as modified nucleobases and nucleobase analogs. In certain embodiments, a nucleobase is optionally substituted A, T, C, G, or U, or an optionally substituted tautomer of A, T, C, G, or U. In certain embodiments, a “nucleobase” refers to a nucleobase unit in an oligonucleotide or a nucleic acid (e.g., A, T, C, G or U as in an oligonucleotide or a nucleic acid).

Nucleoside: The term “nucleoside” refers to a moiety wherein a nucleobase or a modified nucleobase is covalently bound to a sugar or a modified sugar. In certain embodiments, a nucleoside is a natural nucleoside, e.g., adenosine, deoxyadenosine, guanosine, deoxyguanosine, thymidine, uridine, cytidine, or deoxycytidine. In certain embodiments, a nucleoside is a modified nucleoside, e.g., a substituted natural nucleoside selected from adenosine, deoxyadenosine, guanosine, deoxyguanosine, thymidine, uridine, cytidine, and deoxycytidine. In certain embodiments, a nucleoside is a modified nucleoside, e.g., a substituted tautomer of a natural nucleoside selected from adenosine, deoxyadenosine, guanosine, deoxyguanosine, thymidine, uridine, cytidine, and deoxycytidine. In certain embodiments, a “nucleoside” refers to a nucleoside unit in an oligonucleotide or a nucleic acid.

Nucleotide: The term “nucleotide” as used herein refers to a monomeric unit of a polynucleotide that consists of a nucleobase, a sugar, and one or more internucleotidic linkages (e.g., phosphate linkages in natural DNA and RNA). The naturally occurring bases [guanine, (G), adenine, (A), cytosine, (C), thymine, (T), and uracil (U)] are derivatives of purine or pyrimidine, though it should be understood that naturally and non-naturally occurring base analogs are also included. The naturally occurring sugar is the pentose (five-carbon sugar) deoxyribose (which forms DNA) or ribose (which forms RNA), though it should be understood that naturally and non-naturally occurring sugar analogs are also included. Nucleotides are linked via internucleotidic linkages to form nucleic acids, or polynucleotides. Many internucleotidic linkages are known in the art (such as, though not limited to, phosphate, phosphorothioates, boranophosphates and the like). Artificial nucleic acids include PNAs (peptide nucleic acids), phosphotriesters, phosphorothionates, H-phosphonates, phosphoramidates, boranophosphates, methylphosphonates, phosphonoacetates, thiophosphonoacetates and other variants of the phosphate backbone of native nucleic acids, such as those described herein. In certain embodiments, a natural nucleotide comprises a naturally occurring base, sugar and internucleotidic linkage. As used herein, the term “nucleotide” also encompasses structural analogs used in lieu of natural or naturally-occurring nucleotides, such as modified nucleotides and nucleotide analogs. In certain embodiments, a “nucleotide” refers to a nucleotide unit in an oligonucleotide or a nucleic acid.

Oligonucleotide: The term “oligonucleotide” refers to a polymer or oligomer of nucleotides, and may contain any combination of natural and non-natural nucleobases, sugars, and internucleotidic linkages.

Oligonucleotides can be single-stranded or double-stranded. A single-stranded oligonucleotide can have double-stranded regions (formed by two portions of the single-stranded oligonucleotide) and a double-stranded oligonucleotide, which comprises two oligonucleotide chains, can have single-stranded regions for example, at regions where the two oligonucleotide chains are not complementary to each other. Example oligonucleotides include, but are not limited to structural genes, genes including control and termination regions, self-replicating systems such as viral or plasmid DNA, single-stranded and double-stranded RNAi agents and other RNA interference reagents (RNAi agents or iRNA agents), shRNA, antisense oligonucleotides, ribozymes, microRNAs, microRNA mimics, supermirs, aptamers, antimirs, antagomirs, Ul adaptors, triplex-forming oligonucleotides, G-quadruplex oligonucleotides, RNA activators, immuno-stimulatory oligonucleotides, and decoy oligonucleotides.

Oligonucleotides of the present disclosure can be of various lengths. In particular embodiments, oligonucleotides can range from about 2 to about 200 nucleosides in length. In various related embodiments, oligonucleotides, single-stranded, double-stranded, or triple-stranded, can range in length from about 4 to about 10 nucleosides, from about 10 to about 50 nucleosides, from about 20 to about 50 nucleosides, from about 15 to about 30 nucleosides, from about 20 to about 30 nucleosides in length. In certain embodiments, the oligonucleotide is from about 9 to about 39 nucleosides in length. In certain embodiments, the oligonucleotide is at least 4, 5, 6, 7, 8, 9, 10, 11, 12, 13, 14, 15, 16, 17, 18, 19, 20, 21, 22, 23, 24, or 25 nucleosides in length. In certain embodiments, the oligonucleotide is at least 4 nucleosides in length. In certain embodiments, the oligonucleotide is at least 5 nucleosides in length. In certain embodiments, the oligonucleotide is at least 6 nucleosides in length. In certain embodiments, the oligonucleotide is at least 7 nucleosides in length. In certain embodiments, the oligonucleotide is at least 8 nucleosides in length. In certain embodiments, the oligonucleotide is at least 9 nucleosides in length. In certain embodiments, the oligonucleotide is at least 10 nucleosides in length. In certain embodiments, the oligonucleotide is at least 11 nucleosides in length. In certain embodiments, the oligonucleotide is at least 12 nucleosides in length. In certain embodiments, the oligonucleotide is at least 15 nucleosides in length. In certain embodiments, the oligonucleotide is at least 15 nucleosides in length. In certain embodiments, the oligonucleotide is at least 16 nucleosides in length. In certain embodiments, the oligonucleotide is at least 17 nucleosides in length. In certain embodiments, the oligonucleotide is at least 18 nucleosides in length. In certain embodiments, the oligonucleotide is at least 19 nucleosides in length. In certain embodiments, the oligonucleotide is at least 20 nucleosides in length. In certain embodiments, the oligonucleotide is at least 25 nucleosides in length. In certain embodiments, the oligonucleotide is at least 30 nucleosides in length. In certain embodiments, each nucleoside counted in an oligonucleotide length independently comprises a nucleobase comprising a ring having at least one nitrogen ring atom. In certain embodiments, each nucleoside counted in an oligonucleotide length independently comprises A, T, C, G, or U, or optionally substituted A, T, C, G, or U, or an optionally substituted tautomer of A, T, C, G or U.

Oligonucleotide type: As used herein, the phrase “oligonucleotide type” is used to define an oligonucleotide that has a particular base sequence, pattern of backbone linkages (i.e., pattern of internucleotidic linkage types, for example, phosphate, phosphorothioate, phosphorothioate triester, etc.), pattern of backbone chiral centers (i.e., pattern of linkage phosphorus stereochemistry (Rp/Sp)), and pattern of backbone phosphorus modifications. In certain embodiments, oligonucleotides of a common designated “type” are structurally identical to one another.

One of skill in the art will appreciate that synthetic methods of the present disclosure provide for a degree of control during the synthesis of an oligonucleotide strand such that each nucleotide unit of the oligonucleotide strand can be designed and/or selected in advance to have a particular stereochemistry at the linkage phosphorus and/or a particular modification at the linkage phosphorus, and/or a particular base, and/or a particular sugar. In certain embodiments, an oligonucleotide strand is designed and/or selected in advance to have a particular combination of stereocenters at the linkage phosphorus. In certain embodiments, an oligonucleotide strand is designed and/or determined to have a particular combination of modifications at the linkage phosphorus. In certain embodiments, an oligonucleotide strand is designed and/or selected to have a particular combination of bases. In certain embodiments, an oligonucleotide strand is designed and/or selected to have a particular combination of one or more of the above structural characteristics. In certain embodiments, the present disclosure provides compositions comprising or consisting of a plurality of oligonucleotide molecules (e.g., chirally controlled oligonucleotide compositions). In certain embodiments, all such molecules are of the same type (i.e., are structurally identical to one another). In certain embodiments, however, provided compositions comprise a plurality of oligonucleotides of different types, typically in pre-determined relative amounts.

Optionally Substituted: As described herein, compounds, e.g., oligonucleotides, of the disclosure may contain optionally substituted and/or substituted moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. In certain embodiments, an optionally substituted group is unsubstituted. Combinations of substituents envisioned by this disclosure are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein. Certain substituents are described below.

Suitable monovalent substituents on a substitutable atom, e.g., a suitable carbon atom, are independently halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR^{∘}, —SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —Si(R^{∘})₃; —OSi(R^{∘})₃; —B(R^{∘})₂; —OB(R^{∘})₂; —OB(OR^{∘})₂; —P(R^{∘})₂; —P(OR^{∘})₂; —P(R^{∘})(OR^{∘}); —OP(R^{∘})₂; —OP(OR^{∘})₂; —OP(R^{∘})(OR^{∘}); —P(O)(R^{∘})₂; —P(O)(OR^{∘})₂; —OP(O)(R^{∘})₂; —OP(O)(OR^{∘})₂; —OP(O)(OR^{∘})(SR^{∘}); —SP(O)(R^{∘})₂; —SP(O)(OR^{∘})₂; —N(R^{∘})P(O)(R^{∘})₂; —N(R^{∘})P(O)(OR^{∘})₂; —P(R^{∘})₂[B(R^{∘})₃]; —P(OR^{∘})₂[B(R^{∘})₃]; —OP(R^{∘})₂[B(R^{∘})₃]; —OP(OR^{∘})₂[B(R^{∘})₃]; —(C₁₋₄ straight or branched alkylene)O—N(R^{∘})₂; or —(C₁₋₄ straight or branched alkylene)C(O)O—N(R^{∘})₂, wherein each R^{∘} may be substituted as defined herein and is independently hydrogen, C₁₋₂₀ aliphatic, C₁₋₂₀ heteroaliphatic having 1-5 heteroatoms independently selected from nitrogen, oxygen, sulfur, silicon and phosphorus, —CH₂—(C₆₋₁₄ aryl), —O(CH₂)₀₋₁(C₆₋₁₄ aryl), —CH₂-(5-14 membered heteroaryl ring), a 5-20 membered, monocyclic, bicyclic, or polycyclic, saturated, partially unsaturated or aryl ring having 0-5 heteroatoms independently selected from nitrogen, oxygen, sulfur, silicon and phosphorus, or, notwithstanding the definition above, two independent occurrences of R^{∘}, taken together with their intervening atom(s), form a 5-20 membered, monocyclic, bicyclic, or polycyclic, saturated, partially unsaturated or aryl ring having 0-5 heteroatoms independently selected from nitrogen, oxygen, sulfur, silicon and phosphorus, which may be substituted as defined below.

Suitable monovalent substituents on R^{∘} (or the ring formed by taking two independent occurrences of R^{∘} together with their intervening atoms), are independently halogen, —(CH₂)₀₋₂R^{●}, -(haloR^{●}), —(CH₂)₀₋₂OH, —(CH₂)₀₋₂OR^{●}, —(CH₂)₀₋₂CH(OR^{●})₂; —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —NO₂, —SiR^{●}₃, —OSiR^{●}₃, —C(O)SR^{●}, —(C₁₋₄ straight or branched alkylene)C(O)OR^{●}, or —SSR^{●} wherein each R^{●} is unsubstituted or where preceded by “halo” is substituted only with one or more halogens, and is independently selected from C₁₋₄ aliphatic, —CH₂Ph, —O(CH₂)o-1Ph, and a 5-6-membered saturated, partially unsaturated, or aryl ring having 0-4 heteroatoms independently selected from nitrogen, oxygen, and sulfur. Suitable divalent substituents on a saturated carbon atom of R^{∘} include ═O and ═S.

Suitable divalent substituents, e.g., on a suitable carbon atom, are independently the following: ═O, ═S, ═NNR*₂, ═NNHC(O)R*, ═NNHC(O)OR*, =NNHS(O)₂R*, ═NR*, =NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, and an unsubstituted 5-6-membered saturated, partially unsaturated, or aryl ring having 0-4 heteroatoms independently selected from nitrogen, oxygen, and sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, and an unsubstituted 5-6-membered saturated, partially unsaturated, and aryl ring having 0-4 heteroatoms independently selected from nitrogen, oxygen, and sulfur.

Suitable substituents on the aliphatic group of R* are independently halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, and sulfur.

In certain embodiments, suitable substituents on a substitutable nitrogen are independently —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, and sulfur, or, notwithstanding the definition above, two independent occurrences of Rt, taken together with their intervening atom(s) form an unsubstituted 3-12-membered saturated, partially unsaturated, or aryl mono- or bicyclic ring having 0-4 heteroatoms independently selected from nitrogen, oxygen, and sulfur.

Suitable substituents on the aliphatic group of R^{†} are independently halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, and sulfur.

P-modification: as used herein, the term “P-modification” refers to any modification at the linkage phosphorus other than a stereochemical modification. In certain embodiments, a P-modification comprises addition, substitution, or removal of a pendant moiety covalently attached to a linkage phosphorus.

Partially unsaturated: As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

Pharmaceutical composition: As used herein, the term “pharmaceutical composition” refers to an active agent, formulated together with one or more pharmaceutically acceptable carriers. In certain embodiments, an active agent is present in unit dose amount appropriate for administration in a therapeutic regimen that shows a statistically significant probability of achieving a predetermined therapeutic effect when administered to a relevant population. In certain embodiments, pharmaceutical compositions may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces.

Pharmaceutically acceptable: As used herein, the phrase “pharmaceutically acceptable” refers to those compounds, materials, compositions and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Pharmaceutically acceptable carrier: As used herein, the term “pharmaceutically acceptable carrier” means a pharmaceutically-acceptable material, composition or vehicle, such as a liquid or solid filler, diluent, excipient, or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

Pharmaceutically acceptable salt: The term “pharmaceutically acceptable salt”, as used herein, refers to salts of such compounds that are appropriate for use in pharmaceutical contexts, i.e., salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge, et al. describes pharmaceutically acceptable salts in detail in J. Pharmaceutical Sciences, 66: 1-19 (1977). In certain embodiments, pharmaceutically acceptable salt include, but are not limited to, nontoxic acid addition salts, which are salts of an amino group formed with inorganic acids such as hydrochloric acid, hydrobromic acid, phosphoric acid, sulfuric acid and perchloric acid or with organic acids such as acetic acid, maleic acid, tartaric acid, citric acid, succinic acid or malonic acid or by using other methods used in the art such as ion exchange. In certain embodiments, pharmaceutically acceptable salts include, but are not limited to, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. In certain embodiments, a provided compound comprises one or more acidic groups, e.g., an oligonucleotide, and a pharmaceutically acceptable salt is an alkali, alkaline earth metal, or ammonium (e.g., an ammonium salt of N(R)₃, wherein each R is independently defined and described in the present disclosure) salt. Representative alkali or alkaline earth metal salts include sodium, lithium, potassium, calcium, magnesium, and the like. In certain embodiments, a pharmaceutically acceptable salt is a sodium salt. In certain embodiments, a pharmaceutically acceptable salt is a potassium salt. In certain embodiments, a pharmaceutically acceptable salt is a calcium salt. In certain embodiments, pharmaceutically acceptable salts include, when appropriate, nontoxic ammonium, quatemary ammonium, and amine cations formed using counterions such as halide, hydroxide, carboxylate, sulfate, phosphate, nitrate, alkyl having from 1 to 6 carbon atoms, sulfonate and aryl sulfonate. In certain embodiments, a provided compound comprises more than one acid groups, for example, an oligonucleotide may comprise two or more acidic groups (e.g., in natural phosphate linkages and/or modified internucleotidic linkages). In certain embodiments, a pharmaceutically acceptable salt, or generally a salt, of such a compound comprises two or more cations, which can be the same or different. In certain embodiments, in a pharmaceutically acceptable salt (or generally, a salt), all ionizable hydrogen (e.g., in an aqueous solution with a pKa no more than about 11, 10, 9, 8, 7, 6, 5, 4, 3, or 2; in certain embodiments, no more than about 7; in certain embodiments, no more than about 6; in certain embodiments, no more than about 5; in certain embodiments, no more than about 4; in certain embodiments, no more than about 3) in the acidic groups are replaced with cations. In certain embodiments, each phosphorothioate and phosphate group independently exists in its salt form (e.g., if sodium salt, —O—P(O)(SNa)—O— and —O—P(O)(ONa)—O—, respectively). In certain embodiments, each phosphorothioate and phosphate internucleotidic linkage independently exists in its salt form (e.g., if sodium salt, —O—P(O)(SNa)—O— and —O—P(O)(ONa)—O—, respectively). In certain embodiments, a pharmaceutically acceptable salt is a sodium salt of an oligonucleotide. In certain embodiments, a pharmaceutically acceptable salt is a sodium salt of an oligonucleotide, wherein each acidic phosphate and modified phosphate group (e.g., phosphorothioate, phosphate, etc.), if any, exists as a salt form (all sodium salt).

Predetermined: By predetermined (or pre-determined) is meant deliberately selected or non-random or controlled, for example as opposed to randomly occurring, random, or achieved without control. Those of ordinary skill in the art, reading the present specification, will appreciate that the present disclosure provides technologies that permit selection of particular chemistry and/or stereochemistry features to be incorporated into oligonucleotide compositions, and further permits controlled preparation of oligonucleotide compositions having such chemistry and/or stereochemistry features. Such provided compositions are “predetermined” as described herein. Compositions that may contain certain oligonucleotides because they happen to have been generated through a process that are not controlled to intentionally generate the particular chemistry and/or stereochemistry features are not “predetermined” compositions. In certain embodiments, a predetermined composition is one that can be intentionally reproduced (e.g., through repetition of a controlled process). In certain embodiments, a predetermined level of a plurality of oligonucleotides in a composition means that the absolute amount, and/or the relative amount (ratio, percentage, etc.) of the plurality of oligonucleotides in the composition is controlled. In certain embodiments, a predetermined level of a plurality of oligonucleotides in a composition is achieved through chirally controlled oligonucleotide preparation.

Protecting group: The term “protecting group,” as used herein, is well known in the art and includes those described in detail in Protecting Groups in Organic Synthesis, T. W. Greene and P. G. M. Wuts, 3^{rd} edition, John Wiley & Sons, 1999, the entirety of which is incorporated herein by reference. Also included are those protecting groups specially adapted for nucleoside and nucleotide chemistry described in Current Protocols in Nucleic Acid Chemistry, edited by Serge L. Beaucage et al. June 2012, the entirety of Chapter 2 is incorporated herein by reference. Suitable amino-protecting groups include methyl carbamate, ethyl carbamante, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, phenothiazinyl-(10)-carbonyl derivative, N′-p-toluenesulfonylaminocarbonyl derivative, N′-phenylaminothiocarbonyl derivative, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxycarbonylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, 2,4,6-trimethylbenzyl carbamate, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitophenylacetamide, o-nitrophenoxyacetamide, acetoacetamide, (N′-dithiobenzyloxycarbonylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivative, o-nitrobenzamide, o-(benzoyloxymethyl)benzamide, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N—(N′,N′-dimethylaminomethylene)amine, N,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentacarbonylchromium- or tungsten)carbonyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, 3-nitropyridinesulfenamide (Npys), p-toluenesulfonamide (Ts), benzenesulfonamide, 2,3,6,-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), β-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

Suitably protected carboxylic acids further include, but are not limited to, silyl-, alkyl-, alkenyl-, aryl-, and arylalkyl-protected carboxylic acids. Examples of suitable silyl groups include trimethylsilyl, triethylsilyl, t-butyldimethylsilyl, t-butyldiphenylsilyl, triisopropylsilyl, and the like. Examples of suitable alkyl groups include methyl, benzyl, p-methoxybenzyl, 3,4-dimethoxybenzyl, trityl, t-butyl, tetrahydropyran-2-yl. Examples of suitable alkenyl groups include allyl. Examples of suitable aryl groups include optionally substituted phenyl, biphenyl, or naphthyl. Examples of suitable arylalkyl groups include optionally substituted benzyl (e.g., p-methoxybenzyl (MPM), 3,4-dimethoxybenzyl, O-nitrobenzyl, p-nitrobenzyl, p-halobenzyl, 2,6-dichlorobenzyl, p-cyanobenzyl), and 2- and 4-picolyl.

Suitable hydroxyl protecting groups include methyl, methoxylmethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a, 4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl, p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, a-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), alkyl methyl carbonate, 9-fluorenylmethyl carbonate (Fmoc), alkyl ethyl carbonate, alkyl 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), alkyl isobutyl carbonate, alkyl vinyl carbonate alkyl allyl carbonate, alkyl p-nitrophenyl carbonate, alkyl benzyl carbonate, alkyl p-methoxybenzyl carbonate, alkyl 3,4-dimethoxybenzyl carbonate, alkyl o-nitrobenzyl carbonate, alkyl p-nitrobenzyl carbonate, alkyl S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxycarbonyl)benzoate, u-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts). For protecting 1,2- or 1,3-diols, the protecting groups include methylene acetal, ethylidene acetal, 1-t-butylethylidene ketal, 1-phenylethylidene ketal, (4-methoxyphenyl)ethylidene acetal, 2,2,2-trichloroethylidene acetal, acetonide, cyclopentylidene ketal, cyclohexylidene ketal, cycloheptylidene ketal, benzylidene acetal, p-methoxybenzylidene acetal, 2,4-dimethoxybenzylidene ketal, 3,4-dimethoxybenzylidene acetal, 2-nitrobenzylidene acetal, methoxymethylene acetal, ethoxymethylene acetal, dimethoxymethylene ortho ester, 1-methoxyethylidene ortho ester, 1-ethoxyethylidine ortho ester, 1,2-dimethoxyethylidene ortho ester, a-methoxybenzylidene ortho ester, 1-(N,N-dimethylamino)ethylidene derivative, u-(N,N′-dimethylamino)benzylidene derivative, 2-oxacyclopentylidene ortho ester, di-t-butylsilylene group (DTBS), 1,3-(1,1,3,3-tetraisopropyldisiloxanylidene) derivative (TIPDS), tetra-t-butoxydisiloxane-1,3-diylidene derivative (TBDS), cyclic carbonates, cyclic boronates, ethyl boronate, and phenyl boronate.

In certain embodiments, a hydroxyl protecting group is acetyl, t-butyl, t-butoxymethyl, methoxymethyl, tetrahydropyranyl, 1-ethoxyethyl, 1-(2-chloroethoxy)ethyl, 2-trimethylsilylethyl, p-chlorophenyl, 2,4-dinitrophenyl, benzyl, benzoyl, p-phenylbenzoyl, 2,6-dichlorobenzyl, diphenylmethyl, p-nitrobenzyl, triphenylmethyl (trityl), 4,4′-dimethoxytrityl, trimethylsilyl, triethylsilyl, t-butyldimethylsilyl, t-butyldiphenylsilyl, triphenylsilyl, triisopropylsilyl, benzoylformate, chloroacetyl, trichloroacetyl, trifiuoroacetyl, pivaloyl, 9-fluorenylmethyl carbonate, mesylate, tosylate, triflate, trityl, monomethoxytrityl (MMTr), 4,4′-dimethoxytrityl, (DMTr) and 4,4′,4″-trimethoxytrityl (TMTr), 2-cyanoethyl (CE or Cne), 2-(trimethylsilyl)ethyl (TSE), 2-(2-nitrophenyl)ethyl, 2-(4-cyanophenyl)ethyl 2-(4-nitrophenyl)ethyl (NPE), 2-(4-nitrophenylsulfonyl)ethyl, 3,5-dichlorophenyl, 2,4-dimethylphenyl, 2-nitrophenyl, 4-nitrophenyl, 2,4,6-trimethylphenyl, 2-(2-nitrophenyl)ethyl, butylthiocarbonyl, 4,4′,4″-tris(benzoyloxy)trityl, diphenylcarbamoyl, levulinyl, 2-(dibromomethyl)benzoyl (Dbmb), 2-(isopropylthiomethoxymethyl)benzoyl (Ptmt), 9-phenylxanthen-9-yl (pixyl) or 9-(p-methoxyphenyl)xanthine-9-yl (MOX). In certain embodiments, each of the hydroxyl protecting groups is, independently selected from acetyl, benzyl, t-butyldimethylsilyl, t-butyldiphenylsilyl and 4,4′-dimethoxytrityl. In certain embodiments, the hydroxyl protecting group is selected from the group consisting of trityl, monomethoxytrityl and 4,4′-dimethoxytrityl group. In certain embodiments, a phosphorous linkage protecting group is a group attached to the phosphorous linkage (e.g., an internucleotidic linkage) throughout oligonucleotide synthesis. In certain embodiments, a protecting group is attached to a sulfur atom of a phosphorothioate group. In certain embodiments, a protecting group is attached to an oxygen atom of an internucleotide phosphorothioate linkage. In certain embodiments, a protecting group is attached to an oxygen atom of the internucleotide phosphate linkage. In certain embodiments a protecting group is 2-cyanoethyl (CE or Cne), 2-trimethylsilylethyl, 2-nitroethyl, 2-sulfonylethyl, methyl, benzyl, o-nitrobenzyl, 2-(p-nitrophenyl)ethyl (NPE or Npe), 2-phenylethyl, 3-(N-tert-butylcarboxamido)-1-propyl, 4-oxopentyl, 4-methylthio-1-butyl, 2-cyano-1,1-dimethylethyl, 4-N-methylaminobutyl, 3-(2-pyridyl)-1-propyl, 2-[N-methyl-N-(2-pyridyl)]aminoethyl, 2-(N-formyl,N-methyl)aminoethyl, or 4-[N-methyl-N-(2,2,2-trifluoroacetyl)amino]butyl.

Subject: As used herein, the term “subject” or “test subject” refers to any organism to which a compound (e.g., an oligonucleotide) or composition is administered in accordance with the present disclosure e.g., for experimental, diagnostic, prophylactic and/or therapeutic purposes. Typical subjects include animals (e.g., mammals such as mice, rats, rabbits, non-human primates, and humans; insects; worms; etc.) and plants. In certain embodiments, a subject is a human. In certain embodiments, a subject may be suffering from and/or susceptible to a disease, disorder and/or condition.

Substantially: As used herein, the term “substantially” refers to the qualitative condition of exhibiting total or near-total extent or degree of a characteristic or property of interest. A base sequence which is substantially identical or complementary to a second sequence is not fully identical or complementary to the second sequence, but is mostly or nearly identical or complementary to the second sequence. In certain embodiments, an oligonucleotide with a substantially complementary sequence to another oligonucleotide or nucleic acid forms duplex with the oligonucleotide or nucleic acid in a similar fashion as an oligonucleotide with a fully complementary sequence. In addition, one of ordinary skill in the biological and/or chemical arts will understand that biological and chemical phenomena rarely, if ever, go to completion and/or proceed to completeness or achieve or avoid an absolute result. The term “substantially” is therefore used herein to capture the potential lack of completeness inherent in many biological and/or chemical phenomena.

Sugar: The term “sugar” refers to a monosaccharide or polysaccharide in closed and/or open form. In certain embodiments, sugars are monosaccharides. In certain embodiments, sugars are polysaccharides. Sugars include, but are not limited to, ribose, deoxyribose, pentofuranose, pentopyranose, and hexopyranose moieties. As used herein, the term “sugar” also encompasses structural analogs used in lieu of conventional sugar molecules, such as glycol, polymer of which forms the backbone of the nucleic acid analog, glycol nucleic acid (“GNA”), etc. As used herein, the term “sugar” also encompasses structural analogs used in lieu of natural or naturally-occurring nucleotides, such as modified sugars and nucleotide sugars. In certain embodiments, a sugar is an RNA or DNA sugar (ribose or deoxyribose). In certain embodiments, a sugar is a modified ribose or deoxyribose sugar, e.g., 2′-modified, 5′-modified, etc. As described herein, in certain embodiments, when used in oligonucleotides and/or nucleic acids, modified sugars may provide one or more desired properties, activities, etc. In certain embodiments, a sugar is optionally substituted ribose or deoxyribose. In certain embodiments, a “sugar” refers to a sugar unit in an oligonucleotide or a nucleic acid.

Susceptible to: An individual who is “susceptible to” a disease, disorder and/or condition is one who has a higher risk of developing the disease, disorder and/or condition than does a member of the general public. In certain embodiments, an individual who is susceptible to a disease, disorder and/or condition is predisposed to have that disease, disorder and/or condition. In certain embodiments, an individual who is susceptible to a disease, disorder and/or condition may not have been diagnosed with the disease, disorder and/or condition. In certain embodiments, an individual who is susceptible to a disease, disorder and/or condition may exhibit symptoms of the disease, disorder and/or condition. In certain embodiments, an individual who is susceptible to a disease, disorder and/or condition may not exhibit symptoms of the disease, disorder and/or condition. In certain embodiments, an individual who is susceptible to a disease, disorder, and/or condition will develop the disease, disorder, and/or condition. In certain embodiments, an individual who is susceptible to a disease, disorder, and/or condition will not develop the disease, disorder, and/or condition.

Therapeutic agent: As used herein, the term “therapeutic agent” in general refers to any agent that elicits a desired effect (e.g., a desired biological, clinical, or pharmacological effect) when administered to a subject. In certain embodiments, an agent, e.g., a dsRNAi agent, is considered to be a therapeutic agent if it demonstrates a statistically significant effect across an appropriate population. In certain embodiments, an appropriate population is a population of subjects suffering from and/or susceptible to a disease, disorder or condition. In certain embodiments, an appropriate population is a population of model organisms. In certain embodiments, an appropriate population may be defined by one or more criterion such as age group, gender, genetic background, preexisting clinical conditions, prior exposure to therapy. In certain embodiments, a therapeutic agent is a substance that alleviates, ameliorates, relieves, inhibits, prevents, delays onset of, reduces severity of, and/or reduces incidence of one or more hepatic symptoms or features of a disease, disorder, and/or condition in a subject when administered to the subject in an effective amount. In certain embodiments, a “therapeutic agent” is an agent that has been or is required to be approved by a government agency before it can be marketed for administration to humans. In certain embodiments, a “therapeutic agent” is an agent for which a medical prescription is required for administration to humans. In certain embodiments, a therapeutic agent is a provided compound, e.g., a provided oligonucleotide.

Therapeutically effective amount: As used herein, the term “therapeutically effective amount” means an amount of a substance (e.g., a therapeutic agent, composition, and/or formulation) that elicits a desired biological response when administered as part of a therapeutic regimen. In certain embodiments, a therapeutically effective amount of a substance is an amount that is sufficient, when administered to a subject suffering from or susceptible to a disease, disorder, and/or condition, to treat, diagnose, prevent, and/or delay the onset of the disease, disorder, and/or condition. As will be appreciated by those of ordinary skill in this art, the effective amount of a substance may vary depending on such factors as the desired biological endpoint, the substance to be delivered, the target cell or tissue, etc. For example, the effective amount of compound in a formulation to treat a disease, disorder, and/or condition is the amount that alleviates, ameliorates, relieves, inhibits, prevents, delays onset of, reduces severity of and/or reduces incidence of one or more symptoms or features of the disease, disorder, and/or condition. In certain embodiments, a therapeutically effective amount is administered in a single dose; in certain embodiments, multiple unit doses are required to deliver a therapeutically effective amount.

Treat: As used herein, the term “treat,” “treatment,” or “treating” refers to any method used to partially or completely alleviate, ameliorate, relieve, inhibit, prevent, delay onset of, reduce severity of, and/or reduce incidence of one or more symptoms or features of a disease, disorder, and/or condition. Treatment may be administered to a subject who does not exhibit signs of a disease, disorder, and/or condition. In certain embodiments, treatment may be administered to a subject who exhibits only early signs of the disease, disorder, and/or condition, for example for the purpose of decreasing the risk of developing pathology associated with the disease, disorder, and/or condition.

Unsaturated: The term “unsaturated,” as used herein, means that a moiety has one or more units of unsaturation.

Wild-type: As used herein, the term “wild-type” has its art-understood meaning that refers to an entity having a structure and/or activity as found in nature in a “normal” (as contrasted with mutant, diseased, altered, etc.) state or context. Those of ordinary skill in the art will appreciate that wild type genes and polypeptides often exist in multiple different forms (e.g., alleles).

As those skilled in the art will appreciate, methods and compositions described herein relating to provided compounds (e.g., oligonucleotides) generally also apply to pharmaceutically acceptable salts of such compounds.

### 1. Description of Certain Embodiments

Oligonucleotides are useful tools for a wide variety of applications. For example, RNAi oligonucleotides are useful in therapeutic, diagnostic, and research applications, including the treatment of a variety of conditions, disorders, and diseases. The use of naturally occurring nucleic acids (e.g., unmodified DNA or RNA) is limited, for example, by their susceptibility to endo- and exo-nucleases. As such, various synthetic counterparts have been developed to circumvent these shortcomings and/or to further improve various properties and activities. These include synthetic oligonucleotides that contain chemical modifications, e.g., base modifications, sugar modifications, backbone modifications, etc., which, among other things, render these molecules less susceptible to degradation and improve other properties and/or activities. From a structural point of view, modifications to internucleotidic linkages can introduce chirality and/or alter charge, and certain properties may be affected by configurations of linkage phosphorus atoms of oligonucleotides. For example, binding affinity, sequence specific binding to complementary RNA, stability against nucleases, cleavage of target nucleic acids, delivery, pharmacokinetics, etc., can be affected by, inter alia, chirality and/or charge of backbone linkage atoms.

In certain embodiments, the present disclosure demonstrates that compositions comprising ds oligonucleotides (e.g., dsRNAi oligonucleotides, also referred to as dsRNAi agents) with controlled structural elements provide unexpected properties and/or activities.

In certain embodiments, the present disclosure encompasses the recognition that stereochemistry, e.g., stereochemistry of backbone chiral centers, can unexpectedly maintain or improve properties of ds oligonucleotides. For example, but not by way of limitation, the instant disclosure relates, in part, to ds oligonucleotides comprising a guide strand comprising backbone phosphoryl guanidine chiral centers in Sp configuration.

In certain embodiments, the guide strand comprises a backbone phosphoryl guanidine chiral center in the Sp configuration between the +3 nucleotide and the immediately downstream (+4) nucleotide, i.e., in the 3′ direction.

In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, further comprise a phosphoryl guanidine chiral center in the Sp configuration between the +7 and the immediately downstream (+8) nucleotide, between the +10 nucleotide and the immediately downstream (+11) nucleotide, between the +18 nucleotide and the immediately downstream (+19) nucleotide, or combinations thereof.

In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, further comprise a phosphoryl guanidine chiral center in the Rp configuration between the +7 nucleotide and the immediately downstream (+8) nucleotide.

In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, further comprise one or a plurality of modified sugars. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the 5′ terminal (+1) nucleotide, second (+2) nucleotide, third (+3) nucleotide, fourth (+4) nucleotide, fifth (+5) nucleotide, sixth (+6) nucleotide, seventh (+7) nucleotide, eighth (+8) nucleotide, ninth (+9) nucleotide, tenth (+10) nucleotide, twelfth (+12) nucleotide, sixteenth (+16) nucleotide, seventeenth (+17) nucleotide, eighteenth (+18) nucleotide, nineteenth (+19) nucleotide, twentieth (+20) nucleotide, twenty-first (+21) nucleotide, twenty-second (+22), and/or twenty-third (+23), the 3′ terminal, nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the 5′ terminal (+1) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the second (+2) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the third (+3) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the fourth (+4) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the fifth (+5) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the sixth (+6) nucleotide

In certain embodiments, the modified sugar comprises a 2′-F modification, 2′-H modification, 2′-OH modification, 2′-O-alkyl modification, e.g., 2′-O-methyl (OMe) modification, 2′-methoxyethyl (MOE) modification, 5′-alkyl modification, e.g., 5′-(R)-methyl or 5′-(S)-methyl, DNA, locked nucleic acid (LNA), unlocked nucleic acid (UNA), glycol nucleic acid (GNA), or Homo-DNA.

In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the 5′ terminal (+1) nucleotide, second (+2) nucleotide, third (+3) nucleotide, fourth (+4) nucleotide, fifth (+5) nucleotide, and/or sixth (+6) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the 5′ terminal (+1) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the second (+2) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the third (+3) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the fourth (+4) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the fifth (+5) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the sixth (+6) nucleotide.

In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, further comprises one or more of.
(1) a guide strand comprising backbone phosphorothioate chiral centers in Sp configuration between the 3′ terminal nucleotide and the penultimate (N-1) nucleotide and as between the penultimate (N-1) nucleotide and the immediately upstream, i.e., in the 5′ direction, (N-2) nucleotide;
(2) a guide strand comprising backbone phosphorothioate chiral centers in Rp, Sp, or alternating configurations between the 5′ terminal (+1) nucleotide and the immediately downstream, i.e., in the 3′ direction, (+2) nucleotide and between the +2 nucleotide and the immediately downstream (+3) nucleotide;
(3) a guide strand comprising one or more backbone phosphorothioate chiral centers upstream, i.e., in the 5′ direction, relative to backbone phosphorothioate chiral centers in Sp configuration between the 3′ terminal nucleotide and the penultimate (N-1) nucleotide and as between the penultimate (N-1) nucleotide and the immediately upstream (N-2) nucleotide, where the upstream backbone phosphorothioate chiral centers are in Rp or Sp configuration;
(4) a guide strand comprising one or more backbone phosphorothioate chiral centers in Rp or Sp configuration between the 5′ terminal (+1) nucleotide and the immediately downstream (+2) nucleotide; and between the +2 nucleotide and the immediately downstream (+3) nucleotide;
(5) a guide strand comprising one or more backbone phosphorothioate chiral centers in Rp or Sp configuration where linkage occurs between any two adjacent nucleotides between the penultimate 3′ nucleotide of the guide strand, where N is the 3′ terminal nucleotide, and the upstream N-10 nucleotide;
(6) a guide strand comprising one or more backbone phosphoryl guanidine chiral centers in the Rp configuration between the +7 and the immediately downstream (+8), i.e., in the 3′ direction.
(7) a guide strand comprising a 5′ terminal modification;
(8) a passenger strand in combination with one or more of the aforementioned guide strands, comprising one or more backbone chiral centers in Rp or Sp configuration;
(9) a passenger strand in combination with one or more of the aforementioned guide strands, comprising one or more backbone phosphoryl guanidine chiral centers in the Rp configuration between the +7 nucleotide and the immediately downstream (+8) nucleotide, i.e., in the 3′ direction;
(10) a passenger strand in combination with one or more of the aforementioned guide strands, comprising one or more backbone phosphoryl guanidine chiral centers in the Rp configuration between the +15 nucleotide and the immediately downstream (+16) nucleotide, i.e., in the 3′ direction; and
(11) a passenger strand in combination with one or more of the aforementioned guide strands, comprising backbone phosphorothioate chiral centers in the Sp configuration between the 5′ terminal (+1) nucleotide and the immediately downstream, i.e., in the 3′ direction, (+2) nucleotide and between the 3′ terminal nucleotide and the penultimate (N-1) nucleotide;
   wherein the ds oligonucleotide further comprises one or more of:
(1) a guide strand where one or both of the 5′ and 3′ terminal dinucleotides are not linked by a Rp, Sp, or stereorandom non-negatively charged internucleotidic linkages, i.e., the guide strand comprises one or more Rp, Sp, or stereorandom non-negatively charged internucleotidic linkages downstream, i.e., in the 3′ direction, relative to the linkage between the 5′ terminal dinucleotide and/or upstream, i.e., in the 5′ direction, relative to the linkage between the 3′ terminal dinucleotide;
(2) a guide strand where one or more Rp, Sp, or stereorandom non-negatively charged internucleotidic linkage occurs between any two adjacent nucleotides between the second (+2) nucleotide relative to the 5′ terminal nucleotide of the guide strand and the penultimate 3′ (N-1) nucleotide of the guide strand, where N is the 3′ terminal nucleotide;
(3) a guide strand where an Rp, Sp, or stereorandom non-negatively charged internucleotidic linkage occurs between the tenth (+10) and eleventh (+11) nucleotides, relative to the 5′ terminal nucleotide;
(4) a guide strand where an Rp, Sp, or stereorandom non-negatively charged internucleotidic linkage occurs between the seventh (+7) and eighth (+8) nucleotides, relative to the 5′ terminal nucleotide;
(5) a guide strand comprising one or more backbone phosphorothioate chiral centers in Rp or Sp configuration where linkage occurs between any two adjacent nucleotides between the penultimate 3′ nucleotide of the guide strand, where N is the 3′ terminal nucleotide, and the upstream N-10 nucleotide;
(6) a passenger strand where one or more Rp, Sp, or stereorandom non-negatively charged internucleotidic linkage occurs upstream, i.e., in the 5′ direction, relative to the central nucleotide of the passenger strand;
(7) a passenger strand where one or more Rp, Sp, or stereorandom non-negatively charged internucleotidic linkage occurs downstream, i.e., in the 3′ direction, relative to the central nucleotide of the passenger strand;
(8) a passenger strand where one or more backbone phosphorothioate chiral centers in Rp or Sp configuration internucleotidic linkage occurs upstream, i.e., in the 5′ direction, relative to the central nucleotide of the passenger strand;
(9) a passenger strand where one or more backbone phosphorothioate chiral centers in Rp or Sp configuration internucleotidic linkage internucleotidic linkage occurs downstream, i.e., in the 3′ direction, relative to the central nucleotide of the passenger strand; and
(10) a passenger strand comprising one or more modified sugars, e.g. Homo-DNA, between the 5′ terminal (+1) nucleotide and the penultimate (N-1) nucleotide.

In certain embodiments, the ds oligonucleotide further comprises a 2′ modification, e.g., a 2′ F modification, of the 3′ nucleotide of a nucleotide pair linked by a Rp, Sp, or stereorandom non-negatively charged internucleotidic linkage. In certain embodiments, the one or more Rp, Sp, or stereorandom non-negatively charged internucleotidic linkage incorporated into the guide or passenger strand is an Rp non-negatively charged internucleotidic linkage. In certain embodiments, the one or more Rp, Sp, or stereorandom non-negatively charged internucleotidic linkage is an Sp non-negatively charged internucleotidic linkage. In certain embodiments, the one or more Rp, Sp, or stereorandom non-negatively charged internucleotidic linkage is a stereorandom non-negatively charged internucleotidic linkage. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, further comprise one or a plurality of modified sugars. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the 5′ terminal (+1) nucleotide, second (+2) nucleotide, third (+3) nucleotide, fourth (+4) nucleotide, fifth (+5) nucleotide, sixth (+6) nucleotide, seventh (+7) nucleotide, eighth (+8) nucleotide, ninth (+9) nucleotide, tenth (+10) nucleotide, twelfth (+12) nucleotide, sixteenth (+16) nucleotide, seventeenth (+17) nucleotide, eighteenth (+18) nucleotide, nineteenth (+19) nucleotide, twentieth (+20) nucleotide, twenty-first (+21) nucleotide, twenty-second (+22), and/or twenty-third (+23), the 3′ terminal, nucleotide.

In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the 5′ terminal (+1) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the second (+2) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the third (+3) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the fourth (+4) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the fifth (+5) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the sixth (+6) nucleotide.

In certain embodiments, the modified sugar comprises a 2′-F modification, 2′-H modification, 2′-OH modification, 2′-O-alkyl modification, e.g., 2′-O-methyl (OMe) modification, 2′-methoxyethyl (MOE) modification, 5′-alkyl modification, e.g., 5′-(R)-methyl or 5′-(S)-methyl, DNA, locked nucleic acid (LNA), unlocked nucleic acid (UNA), glycol nucleic acid (GNA), or Homo-DNA.

In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the 5′ terminal (+1) nucleotide, second (+2) nucleotide, third (+3) nucleotide, fourth (+4) nucleotide, fifth (+5) nucleotide, and/or sixth (+6) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the 5′ terminal (+1) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the second (+2) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the third (+3) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the fourth (+4) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the fifth (+5) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the sixth (+6) nucleotide.

In certain embodiments, the present disclosure encompasses the recognition that stereochemistry, e.g., stereochemistry of chiral centers at a 5′ terminal modification of guide strands, can unexpectedly maintain or improve properties of the ds oligonucleotides described herein. For example, but not by way of limitation, the instant disclosure relates, in part, to ds oligonucleotides comprising a guide stranding comprising one or more of: (1) a backbone phosphoryl guanidine chiral center in the Sp configuration between the +3 nucleotide and the immediately downstream (+4) nucleotide; (2) a phosphorothioate chiral center in Rp or Sp configuration; (3) an Rp, Sp, or stereorandom non-negatively charged internucleotidic linkage where the 3′ nucleotide of a nucleotide pair linked by an Rp, Sp, or stereorandom non-negatively charged internucleotidic linkage comprises a 2′ modification, e.g., a 2′ F or a 2′-OMe; and (4) a 5′ terminal modification selected from:
(a) 5′ PO modifications, such as, but not limited to:

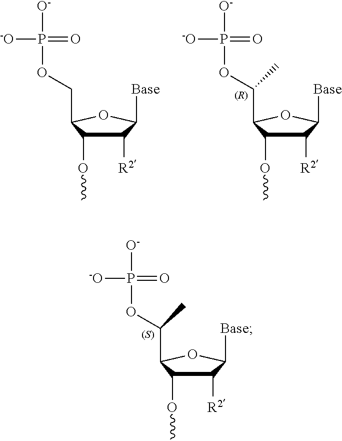

(b) 5′ VP modifications, such as, but not limited to:

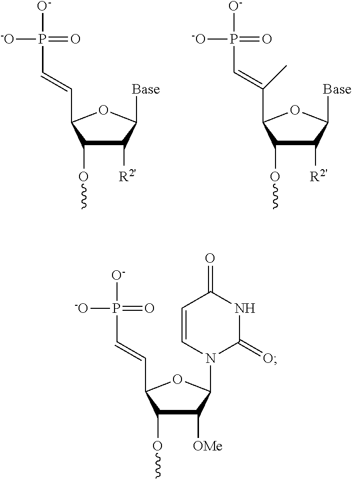

(c) 5′ MeP modifications, such as, but not limited to:

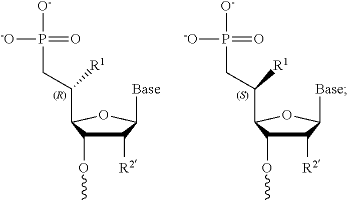

(d) 5′ PN and 5′ Triazole-P modifications, such as, but not limited to:

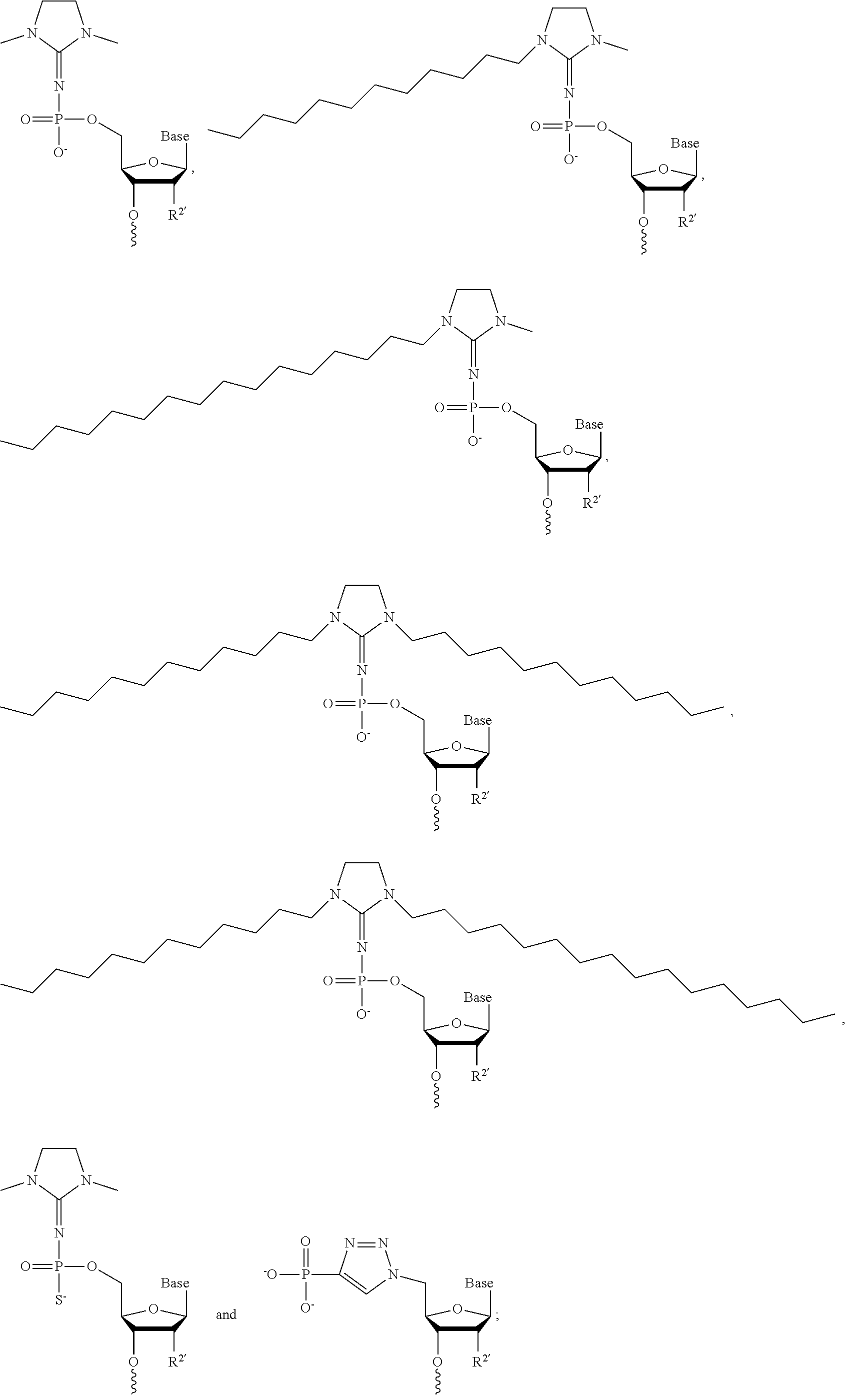

Wherein Base is selected from A, C, G, T, U, abasic and modified nucleobases;
R¹ is selected from an alkyl, methyl, ethyl, isopropyl, propyl, cyclohexyl, benzyl, phenyl, tolyl, xylyl, aryl, or arene group
R^{2′} is selected from H, OH, O-alkyl, O-Me, F, MOE, locked nucleic acid (LNA) bridges and bridged nucleic acid (BNA) bridges to the 4′ C, such as, but not limited to:

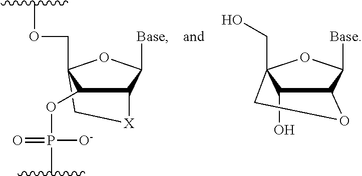

In certain embodiments, the one or more Rp, Sp, or stereorandom non-negatively charged internucleotidic linkage incorporated into the guide strand is an Rp non-negatively charged internucleotidic linkage. In certain embodiments, the one or more Rp, Sp, or stereorandom non-negatively charged internucleotidic linkage is an Sp non-negatively charged internucleotidic linkage. In certain embodiments, the one or more Rp, Sp, or stereorandom non-negatively charged internucleotidic linkage is a stereorandom non-negatively charged internucleotidic linkage.

In certain other embodiments, the present disclosure encompasses the recognition that stereochemistry, e.g., stereochemistry of chiral centers at the 5′ terminal nucleotide of guide strands, can unexpectedly maintain or improve properties of ds oligonucleotides wherein the guide strand of the ds oligonucleotide also comprises a phosphorothioate chiral center in Rp or Sp configuration. For example, but not by way of limitation, the instant disclosure relates, in part, to ds oligonucleotides comprising a guide stranding comprising: (1) a phosphorothioate chiral center in Rp or Sp configuration; (2) an Rp, Sp, or stereorandom non-negatively charged internucleotidic linkage where the 3′ nucleotide of a nucleotide pair linked by an Rp, Sp, or stereorandom-negatively charged internucleotidic linkage comprises a 2′ modification, e.g., a 2′ F or a 2′-OMe; and (3) a 5′ terminal modification selected from:
(a) 5′ PO nucleotides, such as, but not limited to:

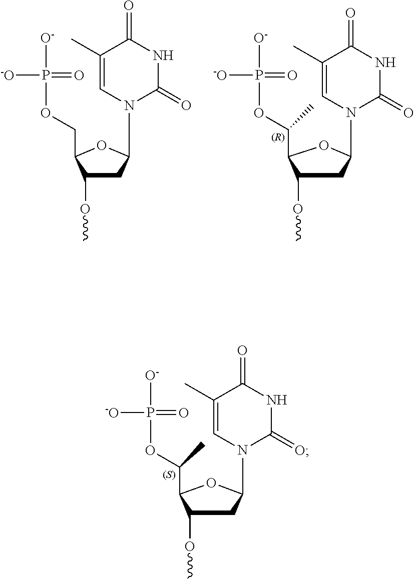

(b) 5 VP nucleotides, such as but not limited to:

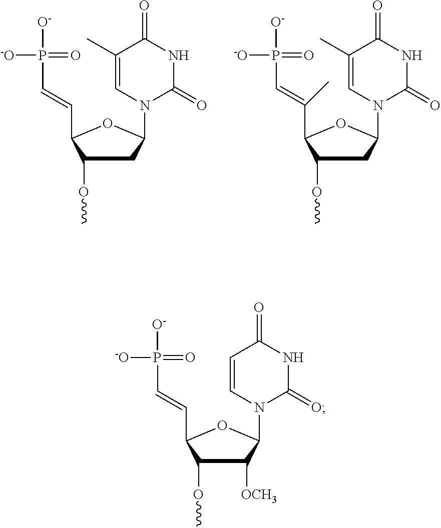

(c) 5′ MeP nucleotides, such as, but not limited to:

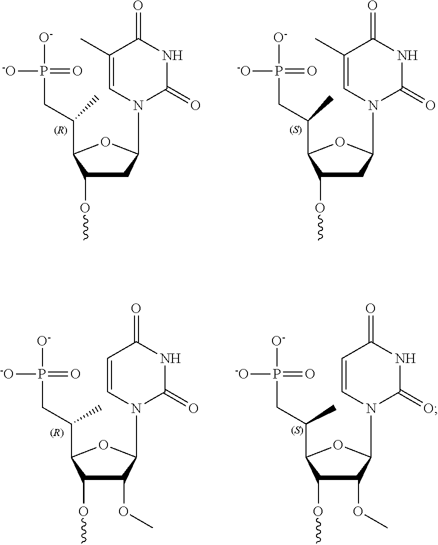

(d) 5′ PN and 5′ Triazole-P nucleotides, such as, but not limited to:

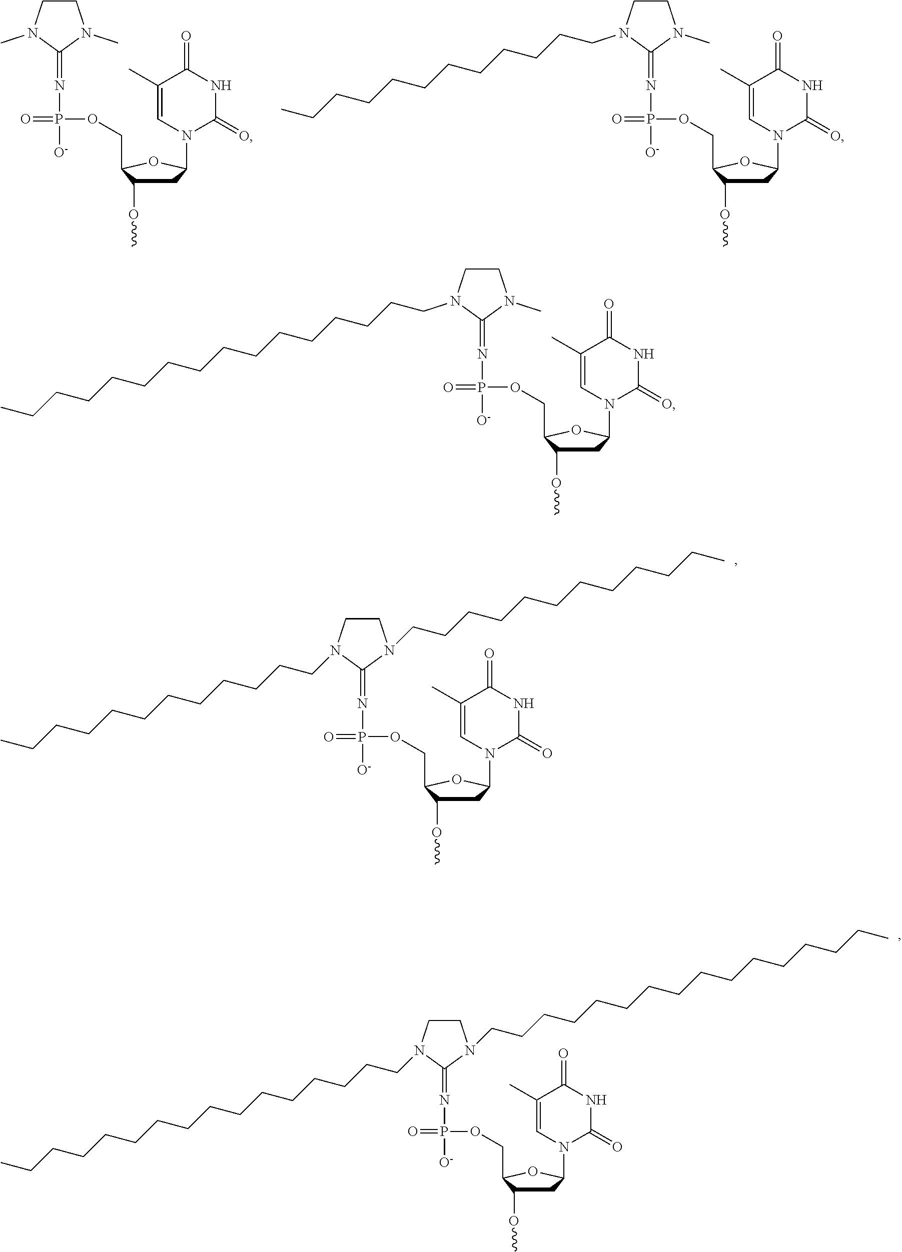

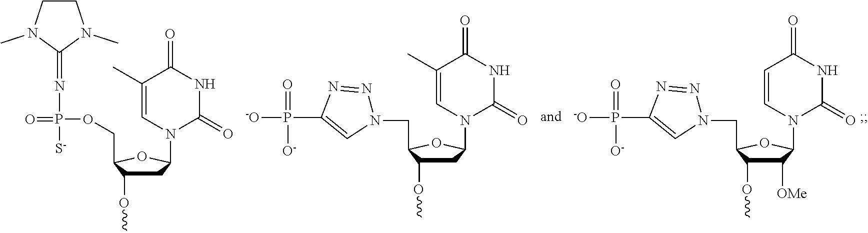

(e) 5′ abasic VP and 5′ abasic MeP nucleotides, such as, but not limited to:

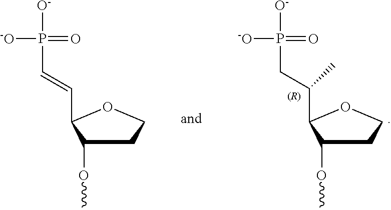

In certain embodiments, the one or more Rp, Sp, or stereorandom non-negatively charged internucleotidic linkage incorporated into the guide strand is an Rp non-negatively charged internucleotidic linkage. In certain embodiments, the one or more Rp, Sp, or stereorandom non-negatively charged internucleotidic linkage is an Sp non-negatively charged internucleotidic linkage. In certain embodiments, the one or more Rp, Sp, or stereorandom non-negatively charged internucleotidic linkage is a stereorandom non-negatively charged internucleotidic linkage.

In certain other embodiments, the present disclosure encompasses the recognition that stereochemistry, e.g., stereochemistry of chiral centers at the 5′ terminal nucleotide of guide strands, can unexpectedly maintain or improve properties of ds oligonucleotides wherein the guide strand of the ds oligonucleotide also comprises a phosphorothioate chiral center in Rp or Sp configuration. For example, but not by way of limitation, the instant disclosure relates, in part, to ds oligonucleotides comprising a guide stranding comprising: (1) a backbone phosphoryl guanidine chiral center in the Sp configuration between the +3 nucleotide and the immediately downstream (+4) nucleotide; (2) a backbone phosphoryl guanidine chiral center in the Rp configuration between the +7 nucleotide and the immediately downstream (+8) nucleotide; (3) a phosphorothioate chiral center in Rp or Sp configuration; (4) an Rp, Sp, or stereorandom non-negatively charged internucleotidic linkage where the 3′ nucleotide of a nucleotide pair linked by an Rp, Sp, or stereorandom non-negatively charged internucleotidic linkage comprises a 2′ modification, e.g., a 2′ F or a 2′-OMe; and (5) a 5′ terminal modification selected from:
(a) 5′ PO nucleotides, such as, but not limited to:

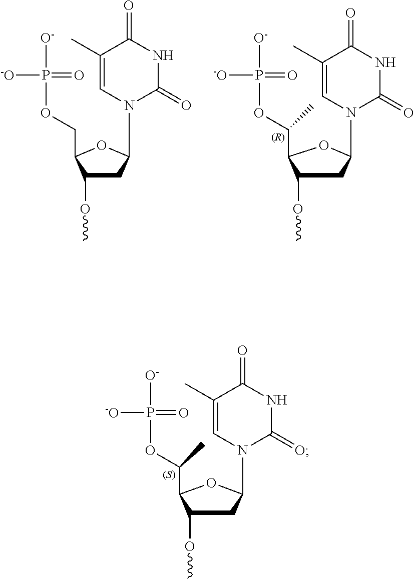

(b) 5′ VP nucleotides, such as, but not limited to:

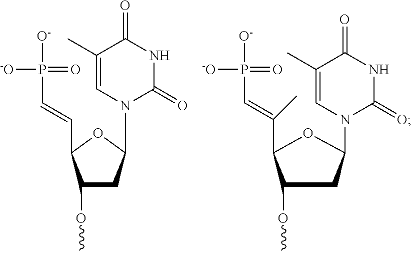

(c) 5′ MeP nucleotides, such as, but not limited to:

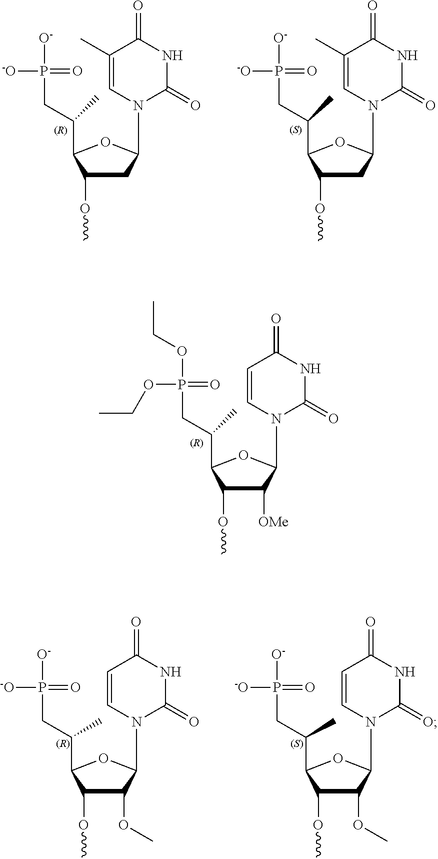

(a) 5′ PN and 5′ Triazole-P nucleotides, such as, but not limited to:

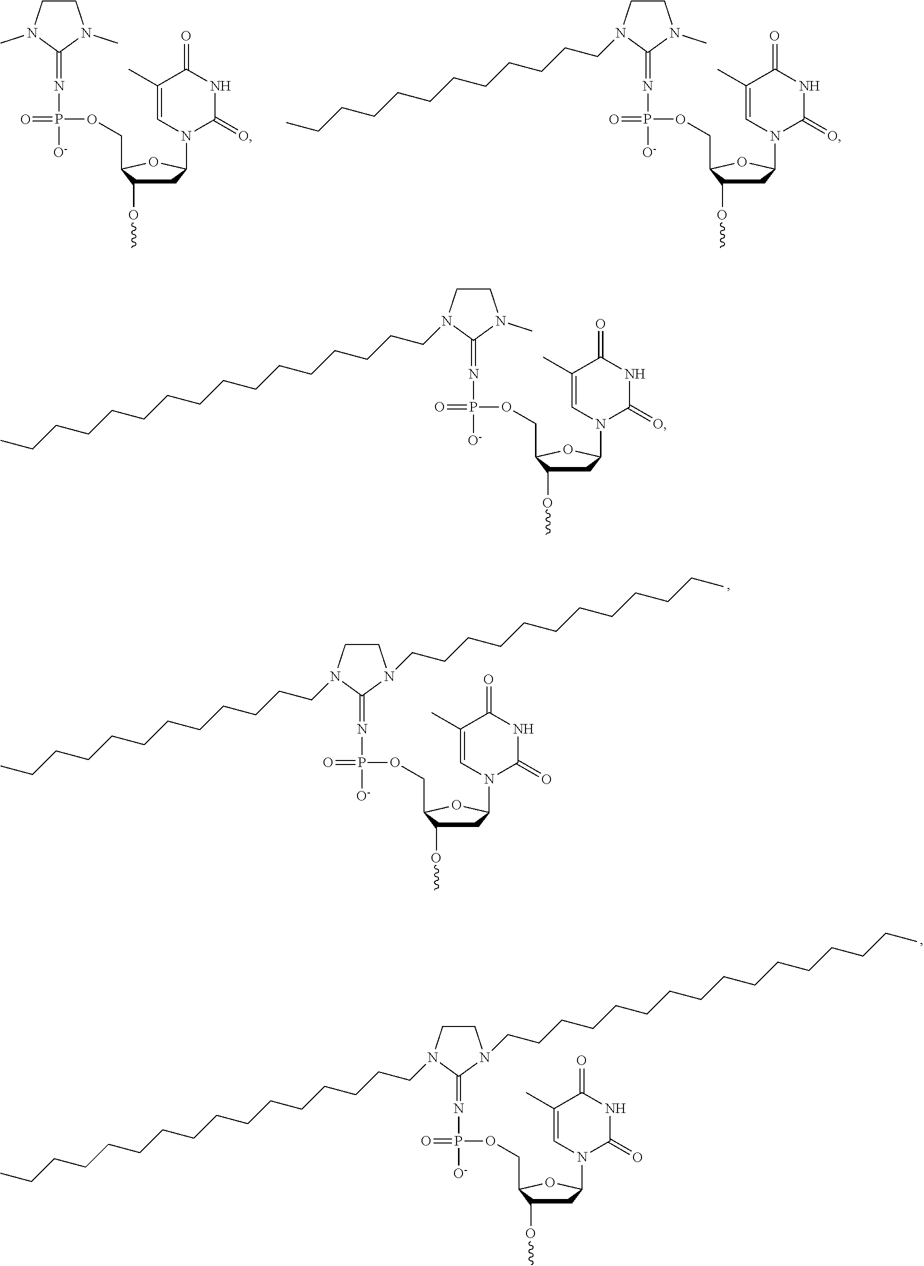

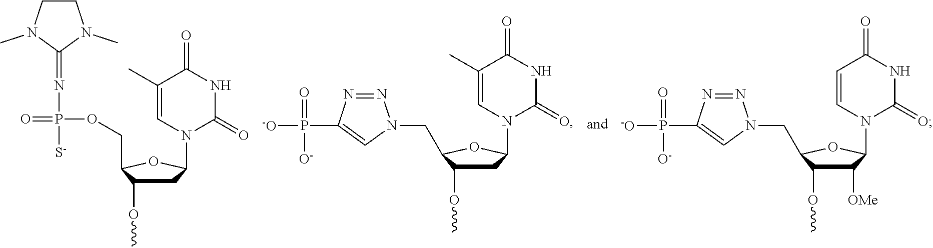

(b) 5′ abasic VP and 5′ abasic MeP nucleotides, such as, but not limited to:

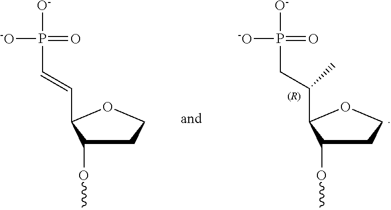

In certain embodiments, the one or more Rp, Sp, or stereorandom non-negatively charged internucleotidic linkage incorporated into the guide strand is an Rp non-negatively charged internucleotidic linkage. In certain embodiments, the one or more Rp, Sp, or stereorandom non-negatively charged internucleotidic linkage is an Sp non-negatively charged internucleotidic linkage. In certain embodiments, the one or more Rp, Sp, or stereorandom non-negatively charged internucleotidic linkage is a stereorandom non-negatively charged internucleotidic linkage.

In certain other embodiments, the present disclosure encompasses the recognition that stereochemistry, e.g., stereochemistry of chiral centers at the 5′ terminal nucleotide of guide strands, can unexpectedly maintain or improve properties of ds oligonucleotides wherein the guide strand of the ds oligonucleotide also comprises a phosphorothioate chiral center in Rp or Sp configuration. For example, but not by way of limitation, the instant disclosure relates, in part, to ds oligonucleotides comprising a guide stranding comprising: (1) a backbone phosphoryl guanidine chiral center in the Sp configuration between the +3 nucleotide and the immediately downstream (+4) nucleotide; (2) a backbone phosphoryl guanidine chiral center in the Rp configuration between the +7 nucleotide and the immediately downstream (+8) nucleotide; (3) a phosphorothioate chiral center in Rp or Sp configuration; and (4) a protected phosphonate selected from:

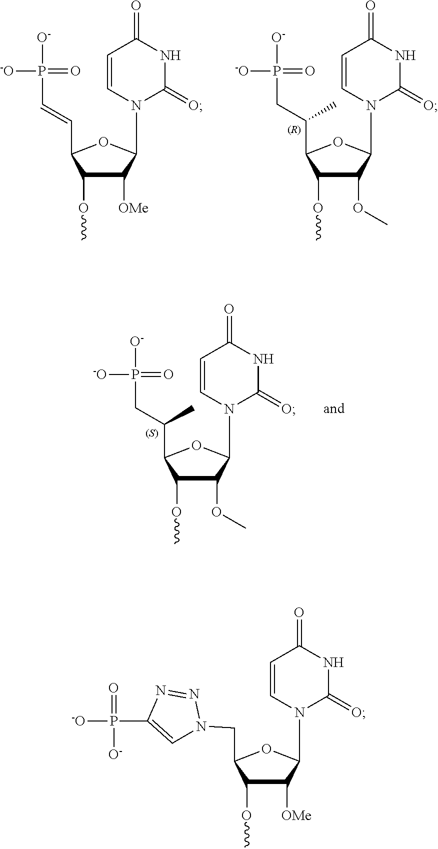

In certain embodiments, the one or more Rp, Sp, or stereorandom non-negatively charged internucleotidic linkage incorporated into the guide strand is an Rp non-negatively charged internucleotidic linkage. In certain embodiments, the one or more Rp, Sp, or stereorandom non-negatively charged internucleotidic linkage is an Sp non-negatively charged internucleotidic linkage. In certain embodiments, the one or more Rp, Sp, or stereorandom non-negatively charged internucleotidic linkage is a stereorandom non-negatively charged internucleotidic linkage. In certain embodiments, the present disclosure encompasses the recognition that non-naturally occurring internucleotidic linkages, e.g., neutral internucleotidic linkages, can, in certain embodiments, be used to link one or more molecules to the double-stranded oligonucleotides described herein. In certain embodiments, such linked molecules can facilitate targeting and/or delivery of the double-stranded oligonucleotide. For example, but not limitation, such linked molecules an include lipophilic molecules. In certain embodiments, the linked molecule is a molecule comprising one or more GalNAc moieties. In certain embodiments, the linked molecule is a receptor. In certain embodiments, the linked molecule is a receptor ligand.

In certain embodiments, the present disclosure provides technologies for incorporating various additional chemical moieties into ds oligonucleotides. In certain embodiments, the present disclosure provides, for example, reagents and methods for introducing additional chemical moieties through nucleobases (e.g., by covalent linkage, optionally via a linker, to a site on a nucleobase).

In certain embodiments, the present disclosure provides technologies, e.g., ds oligonucleotide compositions and methods thereof, that achieve allele-specific suppression, wherein transcripts from one allele of a particular target gene is selectively knocked down relative to at least one other allele of the same gene.

Among other things, the present disclosure provides structural elements, technologies and/or features that can be incorporated into ds oligonucleotides and can impart or tune one or more properties thereof (e.g., relative to an otherwise identical ds oligonucleotide lacking the relevant technology or feature). In certain embodiments, the present disclosure documents that one or more provided technologies and/or features can usefully be incorporated into ds oligonucleotides of various sequences.

In certain embodiments, the present disclosure demonstrates that certain provided structural elements, technologies and/or features are particularly useful for ds oligonucleotides that participate in and/or direct RNAi mechanisms (e.g., RNAi agents). Regardless, however, the teachings of the present disclosure are not limited to ds oligonucleotides that participate in or operate via any particular mechanism. In certain embodiments, the present disclosure pertains to any ds oligonucleotide, useful for any purpose, which operates through any mechanism, and which comprises any sequence, structure or format (or portion thereof) described herein. In certain embodiments, the present disclosure provides a ds oligonucleotide, useful for any purpose, which operates through any mechanism, and which comprises any sequence, structure or format (or portion thereof) described herein, comprising a guide strand comprising backbone phosphoryl guanidine chiral centers in Sp configuration.

In certain embodiments, the guide strand comprises a backbone phosphoryl guanidine chiral center in the Sp configuration between the +3 nucleotide and the immediately downstream (+4) nucleotide, i.e., in the 3′ direction.

In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, further comprise a phosphoryl guanidine chiral center in the Sp configuration between the +7 and the immediately downstream (+8) nucleotide, between the +10 nucleotide and the immediately downstream (+11) nucleotide, between the +18 nucleotide and the immediately downstream (+19) nucleotide, or combinations thereof.

In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, further comprise a phosphoryl guanidine chiral center in the Sp configuration between the +7 and the immediately downstream (+8) nucleotide, between the +15 nucleotide and the immediately downstream (+16) nucleotide, or combinations thereof.

In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, further comprise one or a plurality of modified sugars. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the 5′ terminal (+1) nucleotide, second (+2) nucleotide, third (+3) nucleotide, fourth (+4) nucleotide, fifth (+5) nucleotide, sixth (+6) nucleotide, seventh (+7) nucleotide, eighth (+8) nucleotide, ninth (+9) nucleotide, tenth (+10) nucleotide, twelfth (+12) nucleotide, sixteenth (+16) nucleotide, seventeenth (+17) nucleotide, eighteenth (+18) nucleotide, nineteenth (+19) nucleotide, twentieth (+20) nucleotide, twenty-first (+21) nucleotide, twenty-second (+22), and/or twenty-third (+23), the 3′ terminal, nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the 5′ terminal (+1) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the second (+2) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the third (+3) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the fourth (+4) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the fifth (+5) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the sixth (+6) nucleotide.

In certain embodiments, the modified sugar comprises a 2′-F modification, 2′-H modification, 2′-OH modification, 2′-O-alkyl modification, e.g., 2′-O-methyl (OMe) modification, 2′-methoxyethyl (MOE) modification, 5′-alkyl modification, e.g., 5′-(R)-methyl or 5′-(S)-methyl, DNA, locked nucleic acid (LNA), unlocked nucleic acid (UNA), glycol nucleic acid (GNA), or Homo-DNA.

In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the 5′ terminal (+1) nucleotide, second (+2) nucleotide, third (+3) nucleotide, fourth (+4) nucleotide, fifth (+5) nucleotide, and/or sixth (+6) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the 5′ terminal (+1) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the second (+2) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the third (+3) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the fourth (+4) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the fifth (+5) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the sixth (+6) nucleotide.

In certain embodiments, the present disclosure provides a ds oligonucleotide, useful for any purpose, which operates through any mechanism, and which comprises comprising a guide strand backbone phosphoryl guanidine chiral center in the Sp configuration between the +3 nucleotide and the immediately downstream (+4) nucleotide, i.e., in the 3′ direction, which further comprises:
(1) a guide strand comprising backbone phosphorothioate chiral centers in Sp configuration between the 3′ terminal nucleotide and the penultimate (N-1) nucleotide and as between the penultimate (N-1) nucleotide and the immediately upstream, i.e., in the 5′ direction, (N-2) nucleotide;
(2) a guide strand comprising backbone phosphorothioate chiral centers in Rp, Sp, or alternating configurations between the 5′ terminal (+1) nucleotide and the immediately downstream, i.e., in the 3′ direction, (+2) nucleotide and between the +2 nucleotide and the immediately downstream (+3) nucleotide;
(3) a guide strand comprising one or more backbone phosphorothioate chiral centers upstream, i.e., in the 5′ direction, relative to backbone phosphorothioate chiral centers in Sp configuration between the 3′ terminal nucleotide and the penultimate (N-1) nucleotide and as between the penultimate (N-1) nucleotide and the immediately upstream (N-2) nucleotide, where the upstream backbone phosphorothioate chiral centers are in Rp or Sp configuration;
(4) a guide strand comprising one or more backbone phosphorothioate chiral centers in Rp or Sp configuration between the 5′ terminal (+1) nucleotide and the immediately downstream (+2) nucleotide; and between the +2 nucleotide and the immediately downstream (+3) nucleotide;
(5) a guide strand comprising one or more backbone phosphorothioate chiral centers in Rp or Sp configuration where linkage occurs between any two adjacent nucleotides between the penultimate 3′ nucleotide of the guide strand, where N is the 3′ terminal nucleotide, and the upstream N-10 nucleotide;
(6) a passenger strand in combination with one or more of the aforementioned guide strands, comprising one or more backbone chiral centers in Rp or Sp configuration;
(7) a passenger strand in combination with one or more of the aforementioned guide strands, comprising one or more backbone phosphoryl guanidine chiral centers in the Rp configuration between the +7 nucleotide and the immediately downstream (+8) nucleotide, i.e., in the 3′ direction;
(8) a passenger strand in combination with one or more of the aforementioned guide strands, comprising one or more backbone phosphoryl guanidine chiral centers in the Rp configuration between the +15 nucleotide and the immediately downstream (+16) nucleotide, i.e., in the 3′ direction; and
(9) a passenger strand in combination with one or more of the aforementioned guide strands, comprising backbone phosphorothioate chiral centers in the Sp configuration between the 5′ terminal (+1) nucleotide and the immediately downstream, i.e., in the 3′ direction, (+2) nucleotide and between the 3′ terminal nucleotide and the penultimate (N-1) nucleotide;
   wherein the ds oligonucleotide further comprises one or more of:
(1) a guide strand where one or both of the 5′ and 3′ terminal dinucleotides are not linked by non-negatively charged internucleotidic linkages, i.e., the guide strand comprises one more Rp, Sp, or stereorandom non-negatively charged internucleotidic linkages downstream, i.e., in the 3′ direction, relative to the linkage between the 5′ terminal dinucleotide and/or upstream, i.e., in the 5′ direction, relative to the linkage between the 3′ terminal dinucleotide;
(2) a guide strand where one or more Rp, Sp, or stereorandom non-negatively charged internucleotidic linkage occurs between any two adjacent nucleotides between the second (+2) nucleotide relative to the 5′ terminal nucleotide of the guide strand and the penultimate 3′ (N-1) nucleotide of the guide strand, where N is the 3′ terminal nucleotide;
(3) a guide strand where an Rp, Sp, or stereorandom non-negatively charged internucleotidic linkage occurs between the tenth (+10) and eleventh (+11) nucleotides, relative to the 5′ terminal nucleotide;
(4) a guide strand where an Rp, Sp, or stereorandom non-negatively charged internucleotidic linkage occurs between the seventh (+7) and eighth (+8) nucleotides, relative to the 5′ terminal nucleotide;
(5) a guide strand comprising one or more backbone phosphorothioate chiral centers in Rp or Sp configuration where linkage occurs between any two adjacent nucleotides between the penultimate 3′ nucleotide of the guide strand, where N is the 3′ terminal nucleotide, and the upstream N-10 nucleotide;
(6) a passenger strand where one or more Rp, Sp, or stereorandom non-negatively charged internucleotidic linkage occurs upstream, i.e., in the 5′ direction, relative to the central nucleotide of the passenger strand;
(7) a passenger strand where one or more Rp, Sp, or stereorandom non-negatively charged internucleotidic linkage occurs downstream, i.e., in the 3′ direction, relative to the central nucleotide of the passenger strand;
(8) a passenger strand where one or more backbone phosphorothioate chiral centers in Rp or Sp configuration internucleotidic linkage occurs upstream, i.e., in the 5′ direction, relative to the central nucleotide of the passenger strand;
(9) a passenger strand where one or more backbone phosphorothioate chiral centers in Rp or Sp configuration internucleotidic linkage internucleotidic linkage occurs downstream, i.e., in the 3′ direction, relative to the central nucleotide of the passenger strand; and
(10) a passenger strand comprising one or more modified sugars, e.g. Homo-DNA, between the 5′ terminal (+1) nucleotide and the penultimate (N-1) nucleotide.

In certain embodiments, the ds oligonucleotide further comprises a 2′ modification, e.g., a 2′ F modification, of the 3′ nucleotide of a nucleotide pair linked by an Rp, Sp, or stereorandom non-negatively charged internucleotidic linkage. In certain embodiments, the one or more Rp, Sp, or stereorandom non-negatively charged internucleotidic linkage incorporated into the guide or passenger strand is an Rp non-negatively charged internucleotidic linkage. In certain embodiments, the one or more Rp, Sp, or stereorandom non-negatively charged internucleotidic linkage is an Sp non-negatively charged internucleotidic linkage. In certain embodiments, the one or more Rp, Sp, or stereorandom non-negatively charged internucleotidic linkage is a stereorandom non-negatively charged internucleotidic linkage. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, further comprise one or a plurality of modified sugars. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the 5′ terminal (+1) nucleotide, second (+2) nucleotide, third (+3) nucleotide, fourth (+4) nucleotide, fifth (+5) nucleotide, sixth (+6) nucleotide, seventh (+7) nucleotide, eighth (+8) nucleotide, ninth (+9) nucleotide, tenth (+10) nucleotide, twelfth (+12) nucleotide, sixteenth (+16) nucleotide, seventeenth (+17) nucleotide, eighteenth (+18) nucleotide, nineteenth (+19) nucleotide, twentieth (+20) nucleotide, twenty-first (+21) nucleotide, twenty-second (+22), and/or twenty-third (+23), the 3′ terminal, nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the 5′ terminal (+1) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the second (+2) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the third (+3) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the fourth (+4) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the fifth (+5) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the sixth (+6) nucleotide.

In certain embodiments, the modified sugar comprises a 2′-F modification, 2′-H modification, 2′-OH modification, 2′-O-alkyl modification, e.g., 2′-O-methyl (OMe) modification, 2′-methoxyethyl (MOE) modification, 5′-alkyl modification, e.g., 5′-(R)-methyl or 5′-(S)-methyl, DNA, locked nucleic acid (LNA), unlocked nucleic acid (UNA), glycol nucleic acid (GNA), or Homo-DNA.

In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the 5′ terminal (+1) nucleotide, second (+2) nucleotide, third (+3) nucleotide, fourth (+4) nucleotide, fifth (+5) nucleotide, and/or sixth (+6) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the 5′ terminal (+1) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the second (+2) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the third (+3) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the fourth (+4) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the fifth (+5) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the sixth (+6) nucleotide.

In certain embodiments, the provided ds oligonucleotides may participate in (e.g., direct) RNAi mechanisms. In certain embodiments, provided ds oligonucleotides may participate in RNase H (ribonuclease H) mechanisms. In certain embodiments, provided ds oligonucleotides may act as translational inhibitors (e.g., may provide steric blocks of translation).

In certain embodiments, the ds oligonucleotide comprises a guide strand backbone phosphoryl guanidine chiral center in the Sp configuration between the +3 nucleotide and the immediately downstream (+4) nucleotide, i.e., in the 3′ direction, and the guide strand further comprises backbone phosphorothioate chiral centers in Sp configuration between the 3′ terminal nucleotide and the penultimate (N-1) nucleotide and as between the penultimate (N-1) nucleotide and the immediately upstream (N-2) nucleotide, and one or more of
(1) a guide strand comprising backbone phosphorothioate chiral centers in Sp configuration between the 3′ terminal nucleotide and the penultimate (N-1) nucleotide and as between the penultimate (N-1) nucleotide and the immediately upstream, i.e., in the 5′ direction, (N-2) nucleotide;
(2) a guide strand comprising backbone phosphorothioate chiral centers in Rp, Sp, or alternating configurations between the 5′ terminal (+1) nucleotide and the immediately downstream, i.e., in the 3′ direction, (+2) nucleotide and between the +2 nucleotide and the immediately downstream (+3) nucleotide;
(3) a guide strand comprising one or more backbone phosphorothioate chiral centers upstream, i.e., in the 5′ direction, relative to backbone phosphorothioate chiral centers in Sp configuration between the 3′ terminal nucleotide and the penultimate (N-1) nucleotide and as between the penultimate (N-1) nucleotide and the immediately upstream (N-2) nucleotide, where the upstream backbone phosphorothioate chiral centers are in Rp or Sp configuration;
(4) a guide strand comprising one or more backbone phosphorothioate chiral centers in Rp or Sp configuration between the 5′ terminal (+1) nucleotide and the immediately downstream (+2) nucleotide; and between the +2 nucleotide and the immediately downstream (+3) nucleotide;
(5) a guide strand comprising one or more backbone phosphorothioate chiral centers in Rp or Sp configuration where linkage occurs between any two adjacent nucleotides between the penultimate 3′ nucleotide of the guide strand, where N is the 3′ terminal nucleotide, and the upstream N-10 nucleotide;
(7) a guide strand comprising a 5′ terminal modification;
(8) a passenger strand in combination with one or more of the aforementioned guide strands, comprising one or more backbone chiral centers in Rp or Sp configuration;
(9) a passenger strand in combination with one or more of the aforementioned guide strands, comprising one or more backbone phosphoryl guanidine chiral centers in the Rp configuration between the +7 nucleotide and the immediately downstream (+8) nucleotide, i.e., in the 3′ direction;
(10) a passenger strand in combination with one or more of the aforementioned guide strands, comprising one or more backbone phosphoryl guanidine chiral centers in the Rp configuration between the +15 nucleotide and the immediately downstream (+16) nucleotide, i.e., in the 3′ direction; and
(11) a passenger strand in combination with one or more of the aforementioned guide strands, comprising backbone phosphorothioate chiral centers in the Sp configuration between the 5′ terminal (+1) nucleotide and the immediately downstream, i.e., in the 3′ direction, (+2) nucleotide and between the 3′ terminal nucleotide and the penultimate (N-1) nucleotide;

In certain embodiments, the ds oligonucleotide further comprises a 2′ modification, e.g., a 2′ F modification, of the 3′ nucleotide of a nucleotide pair linked by an Rp, Sp, or stereorandom non-negatively charged internucleotidic linkage, and the passenger strand comprises 0-n Rp, Sp, or stereorandom non-negatively charged internucleotidic linkages, where n is about 1 to 49. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, further comprise one or a plurality of modified sugars. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the 5′ terminal (+1) nucleotide, second (+2) nucleotide, third (+3) nucleotide, fourth (+4) nucleotide, fifth (+5) nucleotide, sixth (+6) nucleotide, seventh (+7) nucleotide, eighth (+8) nucleotide, ninth (+9) nucleotide, tenth (+10) nucleotide, twelfth (+12) nucleotide, sixteenth (+16) nucleotide, seventeenth (+17) nucleotide, eighteenth (+18) nucleotide, nineteenth (+19) nucleotide, twentieth (+20) nucleotide, twenty-first (+21) nucleotide, twenty-second (+22), and/or twenty-third (+23), the 3′ terminal, nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the 5′ terminal (+1) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the second (+2) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the third (+3) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the fourth (+4) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the fifth (+5) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the sixth (+6) nucleotide. In certain embodiments, the modified sugar comprises a 2′-F modification, 2′-H modification, 2′-OH modification, 2′-O-alkyl modification, e.g., 2′-O— methyl (OMe) modification, 2′-methoxyethyl (MOE) modification, 5′-alkyl modification, e.g., 5′-(R)-methyl or 5′-(S)-methyl, DNA, locked nucleic acid (LNA), unlocked nucleic acid (UNA), glycol nucleic acid (GNA), or Homo-DNA. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the 5′ terminal (+1) nucleotide, second (+2) nucleotide, third (+3) nucleotide, fourth (+4) nucleotide, fifth (+5) nucleotide, and/or sixth (+6) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the 5′ terminal (+1) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the second (+2) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the third (+3) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the fourth (+4) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the fifth (+5) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the sixth (+6) nucleotide.

In certain further embodiments, the passenger strand comprises an Sp backbone phosphorothioate chiral center between the 5′ terminal (+1) nucleotide and the immediately downstream (+2) nucleotide and an Sp backbone phosphorothioate chiral center between the penultimate (N-1) nucleotide and the 3′ terminal (N) nucleotide. In certain embodiments, the ds oligonucleotide comprises a guide strand backbone phosphoryl guanidine chiral center in the Sp configuration between the +3 nucleotide and the immediately downstream (+4) nucleotide, i.e., in the 3′ direction, and the guide strand further comprises backbone phosphorothioate chiral centers in Rp, Sp, or alternating configurations between the 5′ terminal (+1) nucleotide and the immediately downstream (+2) nucleotide and between the +2 nucleotide and the immediately downstream (+3) nucleotide, and one or more of:
(1) a guide strand where one or both of the 5′ and 3′ terminal dinucleotides are not linked by non-negatively charged internucleotidic linkages, i.e., the guide strand comprises one more non-negatively charged internucleotidic linkages downstream, i.e., in the 3′ direction, relative to the linkage between the 5′ terminal dinucleotide and/or upstream, i.e., in the 5′ direction, relative to the linkage between the 3′ terminal dinucleotide;
(2) a guide strand where one or more Rp, Sp, or stereorandom non-negatively charged internucleotidic linkage occurs between any two adjacent nucleotides between the second (+2) nucleotide relative to the 5′ terminal nucleotide of the guide strand and the penultimate 3′ (N-1) nucleotide of the guide strand, where N is the 3′ terminal nucleotide;
(3) a guide strand where an Rp, Sp, or stereorandom non-negatively charged internucleotidic linkage occurs between the tenth (+10) and eleventh (+11) nucleotides, relative to the 5′ terminal nucleotide;
(4) a guide strand where an Rp, Sp, or stereorandom non-negatively charged internucleotidic linkage occurs between the seventh (+7) and eighth (+8) nucleotides, relative to the 5′ terminal nucleotide;
(5) a guide strand comprising one or more backbone phosphorothioate chiral centers in Rp or Sp configuration where linkage occurs between any two adjacent nucleotides between the penultimate 3′ nucleotide of the guide strand, where N is the 3′ terminal nucleotide, and the upstream N-10 nucleotide;
(6) a passenger strand where one or more Rp, Sp, or stereorandom non-negatively charged internucleotidic linkage occurs upstream, i.e., in the 5′ direction, relative to the central nucleotide of the passenger strand;
(7) a passenger strand where one or more Rp, Sp, or stereorandom non-negatively charged internucleotidic linkage occurs downstream, i.e., in the 3′ direction, relative to the central nucleotide of the passenger strand;
(8) a passenger strand where one or more backbone phosphorothioate chiral centers in Rp or Sp configuration internucleotidic linkage occurs upstream, i.e., in the 5′ direction, relative to the central nucleotide of the passenger strand;
(9) a passenger strand where one or more backbone phosphorothioate chiral centers in Rp or Sp configuration internucleotidic linkage internucleotidic linkage occurs downstream, i.e., in the 3′ direction, relative to the central nucleotide of the passenger strand; and
(10) a passenger strand comprising one or more modified sugars, e.g. Homo-DNA, between the 5′ terminal (+1) nucleotide and the penultimate (N-1) nucleotide.

In certain embodiments, the ds oligonucleotide further comprises a 2′ modification, e.g., a 2′ F modification, of the 3′ nucleotide of a nucleotide pair linked by an Rp, Sp, or stereorandom non-negatively charged internucleotidic linkage, and the passenger strand comprises 0-n Rp, Sp, or stereorandom non-negatively charged internucleotidic linkages, where n is about 1 to 49.

In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, further comprise one or a plurality of modified sugars. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the 5′ terminal (+1) nucleotide, second (+2) nucleotide, third (+3) nucleotide, fourth (+4) nucleotide, fifth (+5) nucleotide, sixth (+6) nucleotide, seventh (+7) nucleotide, eighth (+8) nucleotide, ninth (+9) nucleotide, tenth (+10) nucleotide, twelfth (+12) nucleotide, sixteenth (+16) nucleotide, seventeenth (+17) nucleotide, eighteenth (+18) nucleotide, nineteenth (+19) nucleotide, twentieth (+20) nucleotide, twenty-first (+21) nucleotide, twenty-second (+22), and/or twenty-third (+23), the 3′ terminal, nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the 5′ terminal (+1) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the second (+2) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the third (+3) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the fourth (+4) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the fifth (+5) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the sixth (+6) nucleotide.

In certain embodiments, the modified sugar comprises a 2′-F modification, 2′-H modification, 2′-OH modification, 2′-O-alkyl modification, e.g., 2′-O-methyl (OMe) modification, 2′-methoxyethyl (MOE) modification, 5′-alkyl modification, e.g., 5′-(R)-methyl or 5′-(S)-methyl, DNA, locked nucleic acid (LNA), unlocked nucleic acid (UNA), glycol nucleic acid (GNA), or Homo-DNA.

In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the 5′ terminal (+1) nucleotide, second (+2) nucleotide, third (+3) nucleotide, fourth (+4) nucleotide, fifth (+5) nucleotide, and/or sixth (+6) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the 5′ terminal (+1) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the second (+2) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the third (+3) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the fourth (+4) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the fifth (+5) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the sixth (+6) nucleotide.

In certain further embodiments, the passenger strand comprises an Sp backbone phosphorothioate chiral center between the 5′ terminal (+1) nucleotide and the immediately downstream (+2) nucleotide, an Rp backbone phosphoryl guanidine between the +7 nucleotide and the immediately downstream (+8) nucleotide, and an Sp backbone phosphorothioate chiral center between the penultimate (N-1) nucleotide and the 3′ terminal (N) nucleotide. In certain embodiments, the ds oligonucleotide comprises a guide strand backbone phosphoryl guanidine chiral center in the Sp configuration between the +3 nucleotide and the immediately downstream (+4) nucleotide, i.e., in the 3′ direction, a guide strand backbone phosphoryl guanidine chiral center in the Rp configuration between the +7 nucleotide and the immediately downstream (+8) nucleotide, i.e., in the 3′ direction and the guide strand further comprises backbone phosphorothioate chiral centers in Rp, Sp, or alternating configurations between the 5′ terminal (+1) nucleotide and the immediately downstream (+2) nucleotide and between the +2 nucleotide and the immediately downstream (+3) nucleotide, and one or more of
(1) a guide strand where one or both of the 5′ and 3′ terminal dinucleotides are not linked by non-negatively charged internucleotidic linkages, i.e., the guide strand comprises one more non-negatively charged internucleotidic linkages downstream, i.e., in the 3′ direction, relative to the linkage between the 5′ terminal dinucleotide and/or upstream, i.e., in the 5′ direction, relative to the linkage between the 3′ terminal dinucleotide;
(2) a guide strand where one or more Rp, Sp, or stereorandom non-negatively charged internucleotidic linkage occurs between any two adjacent nucleotides between the second (+2) nucleotide relative to the 5′ terminal nucleotide of the guide strand and the penultimate 3′ (N-1) nucleotide of the guide strand, where N is the 3′ terminal nucleotide;
(3) a guide strand where an Rp, Sp, or stereorandom non-negatively charged internucleotidic linkage occurs between the tenth (+10) and eleventh (+11) nucleotides, relative to the 5′ terminal nucleotide;
(4) a guide strand where an Rp, Sp, or stereorandom non-negatively charged internucleotidic linkage occurs between the seventh (+7) and eighth (+8) nucleotides, relative to the 5′ terminal nucleotide;
(5) a guide strand comprising one or more backbone phosphorothioate chiral centers in Rp or Sp configuration where linkage occurs between any two adjacent nucleotides between the penultimate 3′ nucleotide of the guide strand, where N is the 3′ terminal nucleotide, and the upstream N-10 nucleotide;
(6) a passenger strand where one or more Rp, Sp, or stereorandom non-negatively charged internucleotidic linkage occurs upstream, i.e., in the 5′ direction, relative to the central nucleotide of the passenger strand;
(7) a passenger strand where one or more Rp, Sp, or stereorandom non-negatively charged internucleotidic linkage occurs downstream, i.e., in the 3′ direction, relative to the central nucleotide of the passenger strand;
(8) a passenger strand where one or more backbone phosphorothioate chiral centers in Rp or Sp configuration internucleotidic linkage occurs upstream, i.e., in the 5′ direction, relative to the central nucleotide of the passenger strand;
(9) a passenger strand where one or more backbone phosphorothioate chiral centers in Rp or Sp configuration internucleotidic linkage internucleotidic linkage occurs downstream, i.e., in the 3′ direction, relative to the central nucleotide of the passenger strand; and
(10) a passenger strand comprising one or more modified sugars, e.g. Homo-DNA, between the 5′ terminal (+1) nucleotide and the penultimate (N-1) nucleotide.

In certain embodiments, the ds oligonucleotide further comprises a 2′ modification, e.g., a 2′ F modification, of the 3′ nucleotide of a nucleotide pair linked by an Rp, Sp, or stereorandom non-negatively charged internucleotidic linkage, and the passenger strand comprises 0-n Rp, Sp, or stereorandom non-negatively charged internucleotidic linkages, where n is about 1 to 49.

In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, further comprise one or a plurality of modified sugars. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the 5′ terminal (+1) nucleotide, second (+2) nucleotide, third (+3) nucleotide, fourth (+4) nucleotide, fifth (+5) nucleotide, sixth (+6) nucleotide, seventh (+7) nucleotide, eighth (+8) nucleotide, ninth (+9) nucleotide, tenth (+10) nucleotide, twelfth (+12) nucleotide, sixteenth (+16) nucleotide, seventeenth (+17) nucleotide, eighteenth (+18) nucleotide, nineteenth (+19) nucleotide, twentieth (+20) nucleotide, twenty-first (+21) nucleotide, twenty-second (+22), and/or twenty-third (+23), the 3′ terminal, nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the 5′ terminal (+1) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the second (+2) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the third (+3) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the fourth (+4) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the fifth (+5) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the sixth (+6) nucleotide. In certain embodiments, the modified sugar comprises a 2′-F modification, 2′-H modification, 2′-OH modification, 2′-O-alkyl modification, e.g., 2′-O— methyl (OMe) modification, 2′-methoxyethyl (MOE) modification, 5′-alkyl modification, e.g., 5′-(R)-methyl or 5′-(S)-methyl, DNA, locked nucleic acid (LNA), unlocked nucleic acid (UNA), glycol nucleic acid (GNA), or Homo-DNA. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the 5′ terminal (+1) nucleotide, second (+2) nucleotide, third (+3) nucleotide, fourth (+4) nucleotide, fifth (+5) nucleotide, and/or sixth (+6) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the 5′ terminal (+1) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the second (+2) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the third (+3) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the fourth (+4) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the fifth (+5) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the sixth (+6) nucleotide.

In certain further embodiments, the passenger strand comprises an Sp backbone phosphorothioate chiral center between the 5′ terminal (+1) nucleotide and the immediately downstream (+2) nucleotide, an Rp backbone phosphoryl guanidine between the +7 nucleotide and the immediately downstream (+8) nucleotide, and an Sp backbone phosphorothioate chiral center between the penultimate (N-1) nucleotide and the 3′ terminal (N) nucleotide. In certain embodiments, the ds oligonucleotide comprises a guide strand backbone phosphoryl guanidine chiral center in the Sp configuration between the +3 nucleotide and the immediately downstream (+4) nucleotide, i.e., in the 3′ direction, a guide strand backbone phosphoryl guanidine chiral center in the Sp configuration between the +7 nucleotide and the immediately downstream (+8) nucleotide, i.e., in the 3′ direction and the guide strand further comprises backbone phosphorothioate chiral centers in Rp, Sp, or alternating configurations between the 5′ terminal (+1) nucleotide and the immediately downstream (+2) nucleotide and between the +2 nucleotide and the immediately downstream (+3) nucleotide, and one or more of:
(1) a guide strand where one or both of the 5′ and 3′ terminal dinucleotides are not linked by non-negatively charged internucleotidic linkages, i.e., the guide strand comprises one more non-negatively charged internucleotidic linkages downstream, i.e., in the 3′ direction, relative to the linkage between the 5′ terminal dinucleotide and/or upstream, i.e., in the 5′ direction, relative to the linkage between the 3′ terminal dinucleotide;
(2) a guide strand where one or more Rp, Sp, or stereorandom non-negatively charged internucleotidic linkage occurs between any two adjacent nucleotides between the second (+2) nucleotide relative to the 5′ terminal nucleotide of the guide strand and the penultimate 3′ (N-1) nucleotide of the guide strand, where N is the 3′ terminal nucleotide;
(3) a guide strand where an Rp, Sp, or stereorandom non-negatively charged internucleotidic linkage occurs between the tenth (+10) and eleventh (+11) nucleotides, relative to the 5′ terminal nucleotide;
(4) a guide strand where an Rp, Sp, or stereorandom non-negatively charged internucleotidic linkage occurs between the seventh (+7) and eighth (+8) nucleotides, relative to the 5′ terminal nucleotide;
(5) a guide strand comprising one or more backbone phosphorothioate chiral centers in Rp or Sp configuration where linkage occurs between any two adjacent nucleotides between the penultimate 3′ nucleotide of the guide strand, where N is the 3′ terminal nucleotide, and the upstream N-10 nucleotide;
(6) a passenger strand where one or more Rp, Sp, or stereorandom non-negatively charged internucleotidic linkage occurs upstream, i.e., in the 5′ direction, relative to the central nucleotide of the passenger strand;
(7) a passenger strand where one or more Rp, Sp, or stereorandom non-negatively charged internucleotidic linkage occurs downstream, i.e., in the 3′ direction, relative to the central nucleotide of the passenger strand;
(8) a passenger strand where one or more backbone phosphorothioate chiral centers in Rp or Sp configuration internucleotidic linkage occurs upstream, i.e., in the 5′ direction, relative to the central nucleotide of the passenger strand;
(9) a passenger strand where one or more backbone phosphorothioate chiral centers in Rp or Sp configuration internucleotidic linkage internucleotidic linkage occurs downstream, i.e., in the 3′ direction, relative to the central nucleotide of the passenger strand; and
(10) a passenger strand comprising one or more modified sugars, e.g. Homo-DNA, between the 5′ terminal (+1) nucleotide and the penultimate (N-1) nucleotide.

In certain embodiments, the ds oligonucleotide further comprises a 2′ modification, e.g., a 2′ F modification, of the 3′ nucleotide of a nucleotide pair linked by an Rp, Sp, or stereorandom non-negatively charged internucleotidic linkage, and the passenger strand comprises 0-n Rp, Sp, or stereorandom non-negatively charged internucleotidic linkages, where n is about 1 to 49.

In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, further comprise one or a plurality of modified sugars. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the 5′ terminal (+1) nucleotide, second (+2) nucleotide, third (+3) nucleotide, fourth (+4) nucleotide, fifth (+5) nucleotide, sixth (+6) nucleotide, seventh (+7) nucleotide, eighth (+8) nucleotide, ninth (+9) nucleotide, tenth (+10) nucleotide, twelfth (+12) nucleotide, sixteenth (+16) nucleotide, seventeenth (+17) nucleotide, eighteenth (+18) nucleotide, nineteenth (+19) nucleotide, twentieth (+20) nucleotide, twenty-first (+21) nucleotide, twenty-second (+22), and/or twenty-third (+23), the 3′ terminal, nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the 5′ terminal (+1) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the second (+2) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the third (+3) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the fourth (+4) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the fifth (+5) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the sixth (+6) nucleotide. In certain embodiments, the modified sugar comprises a 2′-F modification, 2′-H modification, 2′-OH modification, 2′-O-alkyl modification, e.g., 2′-O— methyl (OMe) modification, 2′-methoxyethyl (MOE) modification, 5′-alkyl modification, e.g., 5′-(R)-methyl or 5′-(S)-methyl, DNA, locked nucleic acid (LNA), unlocked nucleic acid (UNA), glycol nucleic acid (GNA), or Homo-DNA. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the 5′ terminal (+1) nucleotide, second (+2) nucleotide, third (+3) nucleotide, fourth (+4) nucleotide, fifth (+5) nucleotide, and/or sixth (+6) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the 5′ terminal (+1) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the second (+2) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the third (+3) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the fourth (+4) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the fifth (+5) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the sixth (+6) nucleotide.

In certain further embodiments, the passenger strand comprises an Sp backbone phosphorothioate chiral center between the 5′ terminal (+1) nucleotide and the immediately downstream (+2) nucleotide and an Rp backbone phosphoryl guanidine between the +7 nucleotide and the immediately downstream (+8) nucleotide. In certain embodiments, the ds oligonucleotide comprises a guide strand backbone phosphoryl guanidine chiral center in the Sp configuration between the +7 nucleotide and the immediately downstream (+8) nucleotide, i.e., in the 3′ direction and the guide strand further comprises backbone phosphorothioate chiral centers in Rp, Sp, or alternating configurations between the 5′ terminal (+1) nucleotide and the immediately downstream (+2) nucleotide and between the +2 nucleotide and the immediately downstream (+3) nucleotide, and one or more of
(1) a guide strand where one or both of the 5′ and 3′ terminal dinucleotides are not linked by non-negatively charged internucleotidic linkages, i.e., the guide strand comprises one more non-negatively charged internucleotidic linkages downstream, i.e., in the 3′ direction, relative to the linkage between the 5′ terminal dinucleotide and/or upstream, i.e., in the 5′ direction, relative to the linkage between the 3′ terminal dinucleotide;
(2) a guide strand where one or more Rp, Sp, or stereorandom non-negatively charged internucleotidic linkage occurs between any two adjacent nucleotides between the second (+2) nucleotide relative to the 5′ terminal nucleotide of the guide strand and the penultimate 3′ (N-1) nucleotide of the guide strand, where N is the 3′ terminal nucleotide;
(3) a guide strand where an Rp, Sp, or stereorandom non-negatively charged internucleotidic linkage occurs between the tenth (+10) and eleventh (+11) nucleotides, relative to the 5′ terminal nucleotide;
(4) a guide strand where an Rp, Sp, or stereorandom non-negatively charged internucleotidic linkage occurs between the seventh (+7) and eighth (+8) nucleotides, relative to the 5′ terminal nucleotide;
(5) a guide strand comprising one or more backbone phosphorothioate chiral centers in Rp or Sp configuration where linkage occurs between any two adjacent nucleotides between the penultimate 3′ nucleotide of the guide strand, where N is the 3′ terminal nucleotide, and the upstream N-10 nucleotide;
(6) a passenger strand where one or more Rp, Sp, or stereorandom non-negatively charged internucleotidic linkage occurs upstream, i.e., in the 5′ direction, relative to the central nucleotide of the passenger strand;
(7) a passenger strand where one or more Rp, Sp, or stereorandom non-negatively charged internucleotidic linkage occurs downstream, i.e., in the 3′ direction, relative to the central nucleotide of the passenger strand;
(8) a passenger strand where one or more backbone phosphorothioate chiral centers in Rp or Sp configuration internucleotidic linkage occurs upstream, i.e., in the 5′ direction, relative to the central nucleotide of the passenger strand;
(9) a passenger strand where one or more backbone phosphorothioate chiral centers in Rp or Sp configuration internucleotidic linkage internucleotidic linkage occurs downstream, i.e., in the 3′ direction, relative to the central nucleotide of the passenger strand; and
(10) a passenger strand comprising one or more modified sugars, e.g. Homo-DNA, between the 5′ terminal (+1) nucleotide and the penultimate (N-1) nucleotide.

In certain embodiments, the ds oligonucleotide further comprises a 2′ modification, e.g., a 2′ F modification, of the 3′ nucleotide of a nucleotide pair linked by an Rp, Sp, or stereorandom non-negatively charged internucleotidic linkage, and the passenger strand comprises 0-n Rp, Sp, or stereorandom non-negatively charged internucleotidic linkages, where n is about 1 to 49.

In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, further comprise one or a plurality of modified sugars. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the 5′ terminal (+1) nucleotide, second (+2) nucleotide, third (+3) nucleotide, fourth (+4) nucleotide, fifth (+5) nucleotide, sixth (+6) nucleotide, seventh (+7) nucleotide, eighth (+8) nucleotide, ninth (+9) nucleotide, tenth (+10) nucleotide, twelfth (+12) nucleotide, sixteenth (+16) nucleotide, seventeenth (+17) nucleotide, eighteenth (+18) nucleotide, nineteenth (+19) nucleotide, twentieth (+20) nucleotide, twenty-first (+21) nucleotide, twenty-second (+22), and/or twenty-third (+23), the 3′ terminal, nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the 5′ terminal (+1) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the second (+2) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the third (+3) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the fourth (+4) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the fifth (+5) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the sixth (+6) nucleotide. In certain embodiments, the modified sugar comprises a 2′-F modification, 2′-H modification, 2′-OH modification, 2′-O-alkyl modification, e.g., 2′-O— methyl (OMe) modification, 2′-methoxyethyl (MOE) modification, 5′-alkyl modification, e.g., 5′-(R)-methyl or 5′-(S)-methyl, DNA, locked nucleic acid (LNA), unlocked nucleic acid (UNA), glycol nucleic acid (GNA), or Homo-DNA. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the 5′ terminal (+1) nucleotide, second (+2) nucleotide, third (+3) nucleotide, fourth (+4) nucleotide, fifth (+5) nucleotide, and/or sixth (+6) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the 5′ terminal (+1) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the second (+2) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the third (+3) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the fourth (+4) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the fifth (+5) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the sixth (+6) nucleotide.

In certain further embodiments, the passenger strand comprises an Sp backbone phosphorothioate chiral center between the 5′ terminal (+1) nucleotide and the immediately downstream (+2) nucleotide, an Rp backbone phosphoryl guanidine between the +7 nucleotide and the immediately downstream (+8) nucleotide, and an Rp backbone phosphoryl guanidine between the +15 nucleotide and the immediately downstream (+16) nucleotide. In certain embodiments, the ds oligonucleotide comprises a guide strand backbone phosphoryl guanidine chiral center in the Sp configuration between the +7 nucleotide and the immediately downstream (+8) nucleotide, i.e., in the 3′ direction and the guide strand further comprises backbone phosphorothioate chiral centers in Rp, Sp, or alternating configurations between the 5′ terminal (+1) nucleotide and the immediately downstream (+2) nucleotide and between the +2 nucleotide and the immediately downstream (+3) nucleotide, and one or more of
(1) a guide strand where one or both of the 5′ and 3′ terminal dinucleotides are not linked by non-negatively charged internucleotidic linkages, i.e., the guide strand comprises one more non-negatively charged internucleotidic linkages downstream, i.e., in the 3′ direction, relative to the linkage between the 5′ terminal dinucleotide and/or upstream, i.e., in the 5′ direction, relative to the linkage between the 3′ terminal dinucleotide;
(2) a guide strand where one or more Rp, Sp, or stereorandom non-negatively charged internucleotidic linkage occurs between any two adjacent nucleotides between the second (+2) nucleotide relative to the 5′ terminal nucleotide of the guide strand and the penultimate 3′ (N-1) nucleotide of the guide strand, where N is the 3′ terminal nucleotide;
(3) a guide strand where an Rp, Sp, or stereorandom non-negatively charged internucleotidic linkage occurs between the tenth (+10) and eleventh (+11) nucleotides, relative to the 5′ terminal nucleotide;
(4) a guide strand where an Rp, Sp, or stereorandom non-negatively charged internucleotidic linkage occurs between the seventh (+7) and eighth (+8) nucleotides, relative to the 5′ terminal nucleotide;
(5) a guide strand comprising one or more backbone phosphorothioate chiral centers in Rp or Sp configuration where linkage occurs between any two adjacent nucleotides between the penultimate 3′ nucleotide of the guide strand, where N is the 3′ terminal nucleotide, and the upstream N-10 nucleotide;
(6) a passenger strand where one or more Rp, Sp, or stereorandom non-negatively charged internucleotidic linkage occurs upstream, i.e., in the 5′ direction, relative to the central nucleotide of the passenger strand;
(7) a passenger strand where one or more Rp, Sp, or stereorandom non-negatively charged internucleotidic linkage occurs downstream, i.e., in the 3′ direction, relative to the central nucleotide of the passenger strand;
(8) a passenger strand where one or more backbone phosphorothioate chiral centers in Rp or Sp configuration internucleotidic linkage occurs upstream, i.e., in the 5′ direction, relative to the central nucleotide of the passenger strand;
(9) a passenger strand where one or more backbone phosphorothioate chiral centers in Rp or Sp configuration internucleotidic linkage internucleotidic linkage occurs downstream, i.e., in the 3′ direction, relative to the central nucleotide of the passenger strand; and
(10) a passenger strand comprising one or more modified sugars, e.g. Homo-DNA, between the 5′ terminal (+1) nucleotide and the penultimate (N-1) nucleotide.

In certain embodiments, the ds oligonucleotide further comprises a 2′ modification, e.g., a 2′ F modification, of the 3′ nucleotide of a nucleotide pair linked by an Rp, Sp, or stereorandom non-negatively charged internucleotidic linkage, and the passenger strand comprises 0-n Rp, Sp, or stereorandom non-negatively charged internucleotidic linkages, where n is about 1 to 49.

In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, further comprise one or a plurality of modified sugars. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the 5′ terminal (+1) nucleotide, second (+2) nucleotide, third (+3) nucleotide, fourth (+4) nucleotide, fifth (+5) nucleotide, sixth (+6) nucleotide, seventh (+7) nucleotide, eighth (+8) nucleotide, ninth (+9) nucleotide, tenth (+10) nucleotide, twelfth (+12) nucleotide, sixteenth (+16) nucleotide, seventeenth (+17) nucleotide, eighteenth (+18) nucleotide, nineteenth (+19) nucleotide, twentieth (+20) nucleotide, twenty-first (+21) nucleotide, twenty-second (+22), and/or twenty-third (+23), the 3′ terminal, nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the 5′ terminal (+1) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the second (+2) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the third (+3) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the fourth (+4) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the fifth (+5) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the sixth (+6) nucleotide. In certain embodiments, the modified sugar comprises a 2′-F modification, 2′-H modification, 2′-OH modification, 2′-O-alkyl modification, e.g., 2′-O— methyl (OMe) modification, 2′-methoxyethyl (MOE) modification, 5′-alkyl modification, e.g., 5′-(R)-methyl or 5′-(S)-methyl, DNA, locked nucleic acid (LNA), unlocked nucleic acid (UNA), glycol nucleic acid (GNA), or Homo-DNA. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the 5′ terminal (+1) nucleotide, second (+2) nucleotide, third (+3) nucleotide, fourth (+4) nucleotide, fifth (+5) nucleotide, and/or sixth (+6) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the 5′ terminal (+1) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the second (+2) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the third (+3) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the fourth (+4) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the fifth (+5) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the sixth (+6) nucleotide.

In certain embodiments, the one or more Rp, Sp, or stereorandom non-negatively charged internucleotidic linkage incorporated into the guide or passenger strand is an Rp non-negatively charged internucleotidic linkage. In certain embodiments, the one or more Rp, Sp, or stereorandom non-negatively charged internucleotidic linkage is an Sp non-negatively charged internucleotidic linkage. In certain embodiments, the one or more Rp, Sp, or stereorandom non-negatively charged internucleotidic linkage is a stereorandom non-negatively charged internucleotidic linkage. In certain further embodiments, the passenger strand comprises an Sp backbone phosphorothioate chiral center between the 5′ terminal (+1) nucleotide and the immediately downstream (+2) nucleotide and an Sp backbone phosphorothioate chiral center between the penultimate (N-1) nucleotide and the 3′ terminal (N) nucleotide.

In certain embodiments, the ds oligonucleotide comprises a guide strand backbone phosphoryl guanidine chiral center in the Sp configuration between the +3 nucleotide and the immediately downstream (+4) nucleotide, i.e., in the 3′ direction, and the guide strand further comprises one or more backbone phosphorothioate chiral centers in Rp or Sp configuration upstream of backbone phosphorothioate chiral centers in Sp configuration between the 3′ terminal nucleotide and the penultimate (N-1) nucleotide and as between the penultimate (N-1) nucleotide and the immediately upstream (N-2) nucleotide, and one or more of:
(1) a guide strand where one or both of the 5′ and 3′ terminal dinucleotides are not linked by non-negatively charged internucleotidic linkages, i.e., the guide strand comprises one more non-negatively charged internucleotidic linkages downstream, i.e., in the 3′ direction, relative to the linkage between the 5′ terminal dinucleotide and/or upstream, i.e., in the 5′ direction, relative to the linkage between the 3′ terminal dinucleotide;
(2) a guide strand where one or more Rp, Sp, or stereorandom non-negatively charged internucleotidic linkage occurs between any two adjacent nucleotides between the second (+2) nucleotide relative to the 5′ terminal nucleotide of the guide strand and the penultimate 3′ (N-1) nucleotide of the guide strand, where N is the 3′ terminal nucleotide;
(3) a guide strand where an Rp, Sp, or stereorandom non-negatively charged internucleotidic linkage occurs between the tenth (+10) and eleventh (+11) nucleotides, relative to the 5′ terminal nucleotide;
(4) a guide strand where an Rp, Sp, or stereorandom non-negatively charged internucleotidic linkage occurs between the seventh (+7) and eighth (+8) nucleotides, relative to the 5′ terminal nucleotide;
(5) a guide strand comprising one or more backbone phosphorothioate chiral centers in Rp or Sp configuration where linkage occurs between any two adjacent nucleotides between the penultimate 3′ nucleotide of the guide strand, where N is the 3′ terminal nucleotide, and the upstream N-10 nucleotide;
(6) a passenger strand where one or more Rp, Sp, or stereorandom non-negatively charged internucleotidic linkage occurs upstream, i.e., in the 5′ direction, relative to the central nucleotide of the passenger strand;
(7) a passenger strand where one or more Rp, Sp, or stereorandom non-negatively charged internucleotidic linkage occurs downstream, i.e., in the 3′ direction, relative to the central nucleotide of the passenger strand;
(8) a passenger strand where one or more backbone phosphorothioate chiral centers in Rp or Sp configuration internucleotidic linkage occurs upstream, i.e., in the 5′ direction, relative to the central nucleotide of the passenger strand;
(9) a passenger strand where one or more backbone phosphorothioate chiral centers in Rp or Sp configuration internucleotidic linkage internucleotidic linkage occurs downstream, i.e., in the 3′ direction, relative to the central nucleotide of the passenger strand; and
(10) a passenger strand comprising one or more modified sugars, e.g. Homo-DNA, between the 5′ terminal (+1) nucleotide and the penultimate (N-1) nucleotide.

In certain embodiments, the ds oligonucleotide further comprises a 2′ modification, e.g., a 2′ F modification, of the 3′ nucleotide of a nucleotide pair linked by an Rp, Sp, or stereorandom non-negatively charged internucleotidic linkage, and the passenger strand comprises 0-n Rp, Sp, or stereorandom non-negatively charged internucleotidic linkages, where n is about 1 to 49.

In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, further comprise one or a plurality of modified sugars. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the 5′ terminal (+1) nucleotide, second (+2) nucleotide, third (+3) nucleotide, fourth (+4) nucleotide, fifth (+5) nucleotide, sixth (+6) nucleotide, seventh (+7)nucleotide, eighth (+8) nucleotide, ninth (+9) nucleotide, tenth (+10) nucleotide, twelfth (+12) nucleotide, sixteenth (+16) nucleotide, seventeenth (+17) nucleotide, eighteenth (+18) nucleotide, nineteenth (+19) nucleotide, twentieth (+20) nucleotide, twenty-first (+21) nucleotide, twenty-second (+22), and/or twenty-third (+23), the 3′ terminal, nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the 5′ terminal (+1) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the second (+2) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the third (+3) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the fourth (+4) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the fifth (+5) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the sixth (+6) nucleotide. In certain embodiments, the modified sugar comprises a 2′-F modification, 2′-H modification, 2′-OH modification, 2′-O-alkyl modification, e.g., 2′-O-methyl (OMe) modification, 2′-methoxyethyl (MOE) modification, 5′-alkyl modification, e.g., 5′-(R)-methyl or 5′-(S)-methyl, DNA, locked nucleic acid (LNA), unlocked nucleic acid (UNA), glycol nucleic acid (GNA), or Homo-DNA. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the 5′ terminal (+1) nucleotide, second (+2) nucleotide, third (+3) nucleotide, fourth (+4) nucleotide, fifth (+5) nucleotide, and/or sixth (+6) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the 5′ terminal (+1) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the second (+2) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the third (+3) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the fourth (+4) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the fifth (+5) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the sixth (+6) nucleotide.

In certain embodiments, the one or more Rp, Sp, or stereorandom non-negatively charged internucleotidic linkage incorporated into the guide or passenger strand is an Rp non-negatively charged internucleotidic linkage. In certain embodiments, the one or more Rp, Sp, or stereorandom non-negatively charged internucleotidic linkage is an Sp non-negatively charged internucleotidic linkage. In certain embodiments, the one or more Rp, Sp, or stereorandom non-negatively charged internucleotidic linkage is a stereorandom non-negatively charged internucleotidic linkage. In certain further embodiments, the passenger strand comprises an Sp backbone phosphorothioate chiral center between the 5′ terminal (+1) nucleotide and the immediately downstream (+2) nucleotide and an Sp backbone phosphorothioate chiral center between the penultimate (N-1) nucleotide and the 3′ terminal (N) nucleotide.

In certain embodiments, the ds oligonucleotide comprises a guide strand backbone phosphoryl guanidine chiral center in the Sp configuration between the +3 nucleotide and the immediately downstream (+4) nucleotide, i.e., in the 3′ direction, and the guide strand further comprises one or more Rp, Sp, or stereorandom non-negatively charged internucleotidic linkage occurs between the second (+2) and third (+3) nucleotides, relative to the 5′ terminal nucleotide, of the guide strand and the internucleotidic linkage to the penultimate 3′ (N-1) nucleotide, and one or more of.
(1) a guide strand where one or both of the 5′ and 3′ terminal dinucleotides are not linked by non-negatively charged internucleotidic linkages, i.e., the guide strand comprises one more non-negatively charged internucleotidic linkages downstream, i.e., in the 3′ direction, relative to the linkage between the 5′ terminal dinucleotide and/or upstream, i.e., in the 5′ direction, relative to the linkage between the 3′ terminal dinucleotide;
(2) a guide strand where one or more Rp, Sp, or stereorandom non-negatively charged internucleotidic linkage occurs between any two adjacent nucleotides between the second (+2) nucleotide relative to the 5′ terminal nucleotide of the guide strand and the penultimate 3′ (N-1) nucleotide of the guide strand, where N is the 3′ terminal nucleotide;
(3) a guide strand where an Rp, Sp, or stereorandom non-negatively charged internucleotidic linkage occurs between the tenth (+10) and eleventh (+11) nucleotides, relative to the 5′ terminal nucleotide;
(4) a guide strand where an Rp, Sp, or stereorandom non-negatively charged internucleotidic linkage occurs between the seventh (+7) and eighth (+8) nucleotides, relative to the 5′ terminal nucleotide;
(5) a guide strand comprising one or more backbone phosphorothioate chiral centers in Rp or Sp configuration where linkage occurs between any two adjacent nucleotides between the penultimate 3′ nucleotide of the guide strand, where N is the 3′ terminal nucleotide, and the upstream N-10 nucleotide;
(6) a passenger strand where one or more Rp, Sp, or stereorandom non-negatively charged internucleotidic linkage occurs upstream, i.e., in the 5′ direction, relative to the central nucleotide of the passenger strand;
(7) a passenger strand where one or more Rp, Sp, or stereorandom non-negatively charged internucleotidic linkage occurs downstream, i.e., in the 3′ direction, relative to the central nucleotide of the passenger strand;
(8) a passenger strand where one or more backbone phosphorothioate chiral centers in Rp or Sp configuration internucleotidic linkage occurs upstream, i.e., in the 5′ direction, relative to the central nucleotide of the passenger strand;
(9) a passenger strand where one or more backbone phosphorothioate chiral centers in Rp or Sp configuration internucleotidic linkage internucleotidic linkage occurs downstream, i.e., in the 3′ direction, relative to the central nucleotide of the passenger strand; and
(10) a passenger strand comprising one or more modified sugars, e.g. Homo-DNA, between the 5′ terminal (+1) nucleotide and the penultimate (N-1) nucleotide.

In certain embodiments, the ds oligonucleotide further comprises a 2′ modification, e.g., a 2′ F modification, of the 3′ nucleotide of a nucleotide pair linked by an Rp, Sp, or stereorandom non-negatively charged internucleotidic linkage, and the passenger strand comprises 0-n Rp, Sp, or stereorandom non-negatively charged internucleotidic linkages, where n is about 1 to 49.

In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, further comprise one or a plurality of modified sugars. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the 5′ terminal (+1) nucleotide, second (+2) nucleotide, third (+3) nucleotide, fourth (+4) nucleotide, fifth (+5) nucleotide, sixth (+6) nucleotide, seventh (+7)nucleotide, eighth (+8) nucleotide, ninth (+9) nucleotide, tenth (+10) nucleotide, twelfth (+12) nucleotide, sixteenth (+16) nucleotide, seventeenth (+17) nucleotide, eighteenth (+18) nucleotide, nineteenth (+19) nucleotide, twentieth (+20) nucleotide, twenty-first (+21) nucleotide, twenty-second (+22), and/or twenty-third (+23), the 3′ terminal, nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the 5′ terminal (+1) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the second (+2) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the third (+3) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the fourth (+4) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the fifth (+5) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the sixth (+6) nucleotide. In certain embodiments, the modified sugar comprises a 2′-F modification, 2′-H modification, 2′-OH modification, 2′-O-alkyl modification, e.g., 2′-O-methyl (OMe) modification, 2′-methoxyethyl (MOE) modification, 5′-alkyl modification, e.g., 5′-(R)-methyl or 5′-(S)-methyl, DNA, locked nucleic acid (LNA), unlocked nucleic acid (UNA), glycol nucleic acid (GNA), or Homo-DNA. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the 5′ terminal (+1) nucleotide, second (+2) nucleotide, third (+3) nucleotide, fourth (+4) nucleotide, fifth (+5) nucleotide, and/or sixth (+6) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the 5′ terminal (+1) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the second (+2) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the third (+3) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the fourth (+4) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the fifth (+5) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the sixth (+6) nucleotide.

In certain embodiments, the one or more Rp, Sp, or stereorandom non-negatively charged internucleotidic linkage incorporated into the guide or passenger strand is an Rp non-negatively charged internucleotidic linkage. In certain embodiments, the one or more Rp, Sp, or stereorandom non-negatively charged internucleotidic linkage is an Sp non-negatively charged internucleotidic linkage. In certain embodiments, the one or more Rp, Sp, or stereorandom non-negatively charged internucleotidic linkage is a stereorandom non-negatively charged internucleotidic linkage. In certain further embodiments, the passenger strand comprises an Sp backbone phosphorothioate chiral center between the 5′ terminal (+1) nucleotide and the immediately downstream (+2) nucleotide and an Sp backbone phosphorothioate chiral center between the penultimate (N-1) nucleotide and the 3′ terminal (N) nucleotide.

In certain embodiments, the ds oligonucleotide comprises a guide strand backbone phosphoryl guanidine chiral center in the Sp configuration between the +3 nucleotide and the immediately downstream (+4) nucleotide, i.e., in the 3′ direction, and the guide strand further comprises backbone phosphorothioate chiral centers in Sp configuration between the 3′ terminal nucleotide and the penultimate (N-1) nucleotide and as between the penultimate (N-1) nucleotide and the immediately upstream (N-2) nucleotide, and one or more of.
(1) a guide strand where one or both of the 5′ and 3′ terminal dinucleotides are not linked by non-negatively charged internucleotidic linkages, i.e., the guide strand comprises one more non-negatively charged internucleotidic linkages downstream, i.e., in the 3′ direction, relative to the linkage between the 5′ terminal dinucleotide and/or upstream, i.e., in the 5′ direction, relative to the linkage between the 3′ terminal dinucleotide;
(2) a guide strand where one or more Rp, Sp, or stereorandom non-negatively charged internucleotidic linkage occurs between any two adjacent nucleotides between the second (+2) nucleotide relative to the 5′ terminal nucleotide of the guide strand and the penultimate 3′ (N-1) nucleotide of the guide strand, where N is the 3′ terminal nucleotide;
(3) a guide strand where an Rp, Sp, or stereorandom non-negatively charged internucleotidic linkage occurs between the tenth (+10) and eleventh (+11) nucleotides, relative to the 5′ terminal nucleotide;
(4) a guide strand where an Rp, Sp, or stereorandom non-negatively charged internucleotidic linkage occurs between the seventh (+7) and eighth (+8) nucleotides, relative to the 5′ terminal nucleotide;
(5) a guide strand comprising one or more backbone phosphorothioate chiral centers in Rp or Sp configuration where linkage occurs between any two adjacent nucleotides between the penultimate 3′ nucleotide of the guide strand, where N is the 3′ terminal nucleotide, and the upstream N-10 nucleotide;
(6) a passenger strand where one or more Rp, Sp, or stereorandom non-negatively charged internucleotidic linkage occurs upstream, i.e., in the 5′ direction, relative to the central nucleotide of the passenger strand;
(7) a passenger strand where one or more Rp, Sp, or stereorandom non-negatively charged internucleotidic linkage occurs downstream, i.e., in the 3′ direction, relative to the central nucleotide of the passenger strand;
(8) a passenger strand where one or more backbone phosphorothioate chiral centers in Rp or Sp configuration internucleotidic linkage occurs upstream, i.e., in the 5′ direction, relative to the central nucleotide of the passenger strand;
(9) a passenger strand where one or more backbone phosphorothioate chiral centers in Rp or Sp configuration internucleotidic linkage internucleotidic linkage occurs downstream, i.e., in the 3′ direction, relative to the central nucleotide of the passenger strand; and
(10) a passenger strand comprising one or more modified sugars, e.g. Homo-DNA, between the 5′ terminal (+1) nucleotide and the penultimate (N-1) nucleotide.

In certain embodiments, the ds oligonucleotide further comprises a 2′ modification, e.g., a 2′ F modification, of the 3′ nucleotide of a nucleotide pair linked by an Rp, Sp, or stereorandom non-negatively charged internucleotidic linkage, and the passenger strand comprises one or more backbone phosphorothioate chiral centers in Rp or Sp configuration. In certain embodiments, the one or more Rp, Sp, or stereorandom non-negatively charged internucleotidic linkage incorporated into the guide or passenger strand is an Rp non-negatively charged internucleotidic linkage. In certain embodiments, the one or more Rp, Sp, or stereorandom non-negatively charged internucleotidic linkage is an Sp non-negatively charged internucleotidic linkage. In certain embodiments, the one or more Rp, Sp, or stereorandom non-negatively charged internucleotidic linkage is a stereorandom non-negatively charged internucleotidic linkage. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, further comprise one or a plurality of modified sugars. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the 5′ terminal (+1) nucleotide, second (+2) nucleotide, third (+3) nucleotide, fourth (+4) nucleotide, fifth (+5) nucleotide, sixth (+6) nucleotide, seventh (+7) nucleotide, eighth (+8) nucleotide, ninth (+9) nucleotide, tenth (+10) nucleotide, twelfth (+12) nucleotide, sixteenth (+16) nucleotide, seventeenth (+17) nucleotide, eighteenth (+18) nucleotide, nineteenth (+19) nucleotide, twentieth (+20) nucleotide, twenty-first (+21) nucleotide, twenty-second (+22), and/or twenty-third (+23), the 3′ terminal, nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the 5′ terminal (+1) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the second (+2) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the third (+3) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the fourth (+4) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the fifth (+5) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the sixth (+6) nucleotide. In certain embodiments, the modified sugar comprises a 2′-F modification, 2′-H modification, 2′-OH modification, 2′-O-alkyl modification, e.g., 2′-O-methyl (OMe) modification, 2′-methoxyethyl (MOE) modification, 5′-alkyl modification, e.g., 5′-(R)-methyl or 5′-(S)-methyl, DNA, locked nucleic acid (LNA), unlocked nucleic acid (UNA), glycol nucleic acid (GNA), or Homo-DNA. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the 5′ terminal (+1) nucleotide, second (+2) nucleotide, third (+3) nucleotide, fourth (+4) nucleotide, fifth (+5) nucleotide, and/or sixth (+6) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the 5′ terminal (+1) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the second (+2) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the third (+3) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the fourth (+4) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the fifth (+5) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the sixth (+6) nucleotide.

In certain embodiments, the ds oligonucleotide comprises a guide strand backbone phosphoryl guanidine chiral center in the Sp configuration between the +3 nucleotide and the immediately downstream (+4) nucleotide, i.e., in the 3′ direction, and the guide strand comprises backbone phosphorothioate chiral centers in Rp, Sp, or alternating configurations between the 5′ terminal (+1) nucleotide and the immediately downstream (+2) nucleotide and between the +2 nucleotide and the immediately downstream (+3) nucleotide, and one or more of:
(1) a guide strand where one or both of the 5′ and 3′ terminal dinucleotides are not linked by non-negatively charged internucleotidic linkages, i.e., the guide strand comprises one more non-negatively charged internucleotidic linkages downstream, i.e., in the 3′ direction, relative to the linkage between the 5′ terminal dinucleotide and/or upstream, i.e., in the 5′ direction, relative to the linkage between the 3′ terminal dinucleotide;
(2) a guide strand where one or more Rp, Sp, or stereorandom non-negatively charged internucleotidic linkage occurs between any two adjacent nucleotides between the second (+2) nucleotide relative to the 5′ terminal nucleotide of the guide strand and the penultimate 3′ (N-1) nucleotide of the guide strand, where N is the 3′ terminal nucleotide;
(3) a guide strand where an Rp, Sp, or stereorandom non-negatively charged internucleotidic linkage occurs between the tenth (+10) and eleventh (+11) nucleotides, relative to the 5′ terminal nucleotide;
(4) a guide strand where an Rp, Sp, or stereorandom non-negatively charged internucleotidic linkage occurs between the seventh (+7) and eighth (+8) nucleotides, relative to the 5′ terminal nucleotide;
(5) a guide strand comprising one or more backbone phosphorothioate chiral centers in Rp or Sp configuration where linkage occurs between any two adjacent nucleotides between the penultimate 3′ nucleotide of the guide strand, where N is the 3′ terminal nucleotide, and the upstream N-10 nucleotide;
(6) a passenger strand where one or more Rp, Sp, or stereorandom non-negatively charged internucleotidic linkage occurs upstream, i.e., in the 5′ direction, relative to the central nucleotide of the passenger strand;
(7) a passenger strand where one or more Rp, Sp, or stereorandom non-negatively charged internucleotidic linkage occurs downstream, i.e., in the 3′ direction, relative to the central nucleotide of the passenger strand;
(8) a passenger strand where one or more backbone phosphorothioate chiral centers in Rp or Sp configuration internucleotidic linkage occurs upstream, i.e., in the 5′ direction, relative to the central nucleotide of the passenger strand;
(9) a passenger strand where one or more backbone phosphorothioate chiral centers in Rp or Sp configuration internucleotidic linkage internucleotidic linkage occurs downstream, i.e., in the 3′ direction, relative to the central nucleotide of the passenger strand; and
(10) a passenger strand comprising one or more modified sugars, e.g. Homo-DNA, between the 5′ terminal (+1) nucleotide and the penultimate (N-1) nucleotide.
   wherein the ds oligonucleotide further comprises a 2′ modification, e.g., a 2′ F modification, of the 3′ nucleotide of a nucleotide pair linked by an Rp, Sp, or stereorandom non-negatively charged internucleotidic linkage, and the passenger strand comprises one or more backbone chiral centers in Rp or Sp configuration. In certain embodiments, the one or more Rp, Sp, or stereorandom non-negatively charged internucleotidic linkage incorporated into the guide or passenger strand is an Rp non-negatively charged internucleotidic linkage. In certain embodiments, the one or more Rp, Sp, or stereorandom non-negatively charged internucleotidic linkage is an Sp non-negatively charged internucleotidic linkage. In certain embodiments, the one or more Rp, Sp, or stereorandom non-negatively charged internucleotidic linkage is a stereorandom non-negatively charged internucleotidic linkage. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, further comprise one or a plurality of modified sugars. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the 5′ terminal (+1) nucleotide, second (+2) nucleotide, third (+3) nucleotide, fourth (+4) nucleotide, fifth (+5) nucleotide, sixth (+6) nucleotide, seventh (+7)nucleotide, eighth (+8) nucleotide, ninth (+9) nucleotide, tenth (+10) nucleotide, twelfth (+12) nucleotide, sixteenth (+16) nucleotide, seventeenth (+17) nucleotide, eighteenth (+18) nucleotide, nineteenth (+19) nucleotide, twentieth (+20) nucleotide, twenty-first (+21) nucleotide, twenty-second (+22), and/or twenty-third (+23), the 3′ terminal, nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the 5′ terminal (+1) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the second (+2) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the third (+3) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the fourth (+4) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the fifth (+5) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the sixth (+6) nucleotide. In certain embodiments, the modified sugar comprises a 2′-F modification, 2′-H modification, 2′-OH modification, 2′-O-alkyl modification, e.g., 2′-O-methyl (OMe) modification, 2′-methoxyethyl (MOE) modification, 5′-alkyl modification, e.g., 5′-(R)-methyl or 5′-(S)-methyl, DNA, locked nucleic acid (LNA), unlocked nucleic acid (UNA), glycol nucleic acid (GNA), or Homo-DNA. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the 5′ terminal (+1) nucleotide, second (+2) nucleotide, third (+3) nucleotide, fourth (+4) nucleotide, fifth (+5) nucleotide, and/or sixth (+6) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the 5′ terminal (+1) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the second (+2) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the third (+3) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the fourth (+4) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the fifth (+5) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the sixth (+6) nucleotide.

In certain embodiments, the ds oligonucleotide comprises a guide strand backbone phosphoryl guanidine chiral center in the Sp configuration between the +3 nucleotide and the immediately downstream (+4) nucleotide, i.e., in the 3′ direction, and the guide strand further comprises one or more backbone phosphorothioate chiral centers in Rp or Sp configuration upstream of backbone chiral centers in Sp configuration between the 3′ terminal nucleotide and the penultimate (N-1) nucleotide and as between the penultimate (N-1) nucleotide and the immediately upstream (N-2) nucleotide, and one or more of.
(1) a guide strand where one or both of the 5′ and 3′ terminal dinucleotides are not linked by non-negatively charged internucleotidic linkages, i.e., the guide strand comprises one more non-negatively charged internucleotidic linkages downstream, i.e., in the 3′ direction, relative to the linkage between the 5′ terminal dinucleotide and/or upstream, i.e., in the 5′ direction, relative to the linkage between the 3′ terminal dinucleotide;
(2) a guide strand where one or more Rp, Sp, or stereorandom non-negatively charged internucleotidic linkage occurs between any two adjacent nucleotides between the second (+2) nucleotide relative to the 5′ terminal nucleotide of the guide strand and the penultimate 3′ (N-1) nucleotide of the guide strand, where N is the 3′ terminal nucleotide;
(3) a guide strand where an Rp, Sp, or stereorandom non-negatively charged internucleotidic linkage occurs between the tenth (+10) and eleventh (+11) nucleotides, relative to the 5′ terminal nucleotide;
(4) a guide strand where an Rp, Sp, or stereorandom non-negatively charged internucleotidic linkage occurs between the seventh (+7) and eighth (+8) nucleotides, relative to the 5′ terminal nucleotide;
(5) a guide strand comprising one or more backbone phosphorothioate chiral centers in Rp or Sp configuration where linkage occurs between any two adjacent nucleotides between the penultimate 3′ nucleotide of the guide strand, where N is the 3′ terminal nucleotide, and the upstream N-10 nucleotide;
(6) a passenger strand where one or more Rp, Sp, or stereorandom non-negatively charged internucleotidic linkage occurs upstream, i.e., in the 5′ direction, relative to the central nucleotide of the passenger strand;
(7) a passenger strand where one or more Rp, Sp, or stereorandom non-negatively charged internucleotidic linkage occurs downstream, i.e., in the 3′ direction, relative to the central nucleotide of the passenger strand;
(8) a passenger strand where one or more backbone phosphorothioate chiral centers in Rp or Sp configuration internucleotidic linkage occurs upstream, i.e., in the 5′ direction, relative to the central nucleotide of the passenger strand;
(9) a passenger strand where one or more backbone phosphorothioate chiral centers in Rp or Sp configuration internucleotidic linkage internucleotidic linkage occurs downstream, i.e., in the 3′ direction, relative to the central nucleotide of the passenger strand; and
(10) a passenger strand comprising one or more modified sugars, e.g. Homo-DNA, between the 5′ terminal (+1) nucleotide and the penultimate (N-1) nucleotide.

In certain embodiments, the ds oligonucleotide further comprises a 2′ modification, e.g., a 2′ F modification, of the 3′ nucleotide of a nucleotide pair linked by an Rp, Sp, or stereorandom non-negatively charged internucleotidic linkage, and the passenger strand comprises one or more backbone chiral centers in Rp or Sp configuration. In certain embodiments, the one or more Rp, Sp, or stereorandom non-negatively charged internucleotidic linkage incorporated into the guide or passenger strand is an Rp non-negatively charged internucleotidic linkage. In certain embodiments, the one or more Rp, Sp, or stereorandom non-negatively charged internucleotidic linkage is an Sp non-negatively charged internucleotidic linkage. In certain embodiments, the one or more Rp, Sp, or stereorandom non-negatively charged internucleotidic linkage is a stereorandom non-negatively charged internucleotidic linkage. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, further comprise one or a plurality of modified sugars. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the 5′ terminal (+1) nucleotide, second (+2) nucleotide, third (+3) nucleotide, fourth (+4) nucleotide, fifth (+5) nucleotide, sixth (+6) nucleotide, seventh (+7) nucleotide, eighth (+8) nucleotide, ninth (+9) nucleotide, tenth (+10) nucleotide, twelfth (+12) nucleotide, sixteenth (+16) nucleotide, seventeenth (+17) nucleotide, eighteenth (+18) nucleotide, nineteenth (+19) nucleotide, twentieth (+20) nucleotide, twenty-first (+21) nucleotide, twenty-second (+22), and/or twenty-third (+23), the 3′ terminal, nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the 5′ terminal (+1) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the second (+2) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the third (+3) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the fourth (+4) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the fifth (+5) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the sixth (+6) nucleotide. In certain embodiments, the modified sugar comprises a 2′-F modification, 2′-H modification, 2′-OH modification, 2′-O-alkyl modification, e.g., 2′-O-methyl (OMe) modification, 2′-methoxyethyl (MOE) modification, 5′-alkyl modification, e.g., 5′-(R)-methyl or 5′-(S)-methyl, DNA, locked nucleic acid (LNA), unlocked nucleic acid (UNA), glycol nucleic acid (GNA), or Homo-DNA. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the 5′ terminal (+1) nucleotide, second (+2) nucleotide, third (+3) nucleotide, fourth (+4) nucleotide, fifth (+5) nucleotide, and/or sixth (+6) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the 5′ terminal (+1) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the second (+2) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the third (+3) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the fourth (+4) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the fifth (+5) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the sixth (+6) nucleotide.

In certain embodiments, the ds oligonucleotide comprises a guide strand backbone phosphoryl guanidine chiral center in the Sp configuration between the +3 nucleotide and the immediately downstream (+4) nucleotide, i.e., in the 3′ direction, and the guide strand further comprises one or more backbone phosphorothioate chiral centers in Rp or Sp configuration between the 5′ terminal (+1) nucleotide and the immediately downstream (+2) nucleotide and between the (+2) nucleotide and the immediately downstream (+3) nucleotide, as well as between the (+5) nucleotide and the (+6) nucleotide, and one or more of
(1) a guide strand where one or both of the 5′ and 3′ terminal dinucleotides are not linked by non-negatively charged internucleotidic linkages, i.e., the guide strand comprises one more non-negatively charged internucleotidic linkages downstream, i.e., in the 3′ direction, relative to the linkage between the 5′ terminal dinucleotide and/or upstream, i.e., in the 5′ direction, relative to the linkage between the 3′ terminal dinucleotide;
(2) a guide strand where one or more Rp, Sp, or stereorandom non-negatively charged internucleotidic linkage occurs between any two adjacent nucleotides between the second (+2) nucleotide relative to the 5′ terminal nucleotide of the guide strand and the penultimate 3′ (N-1) nucleotide of the guide strand, where N is the 3′ terminal nucleotide;
(3) a guide strand where an Rp, Sp, or stereorandom non-negatively charged internucleotidic linkage occurs between the tenth (+10) and eleventh (+11) nucleotides, relative to the 5′ terminal nucleotide;
(4) a guide strand where an Rp, Sp, or stereorandom non-negatively charged internucleotidic linkage occurs between the seventh (+7) and eighth (+8) nucleotides, relative to the 5′ terminal nucleotide;
(5) a guide strand comprising one or more backbone phosphorothioate chiral centers in Rp or Sp configuration where linkage occurs between any two adjacent nucleotides between the penultimate 3′ nucleotide of the guide strand, where N is the 3′ terminal nucleotide, and the upstream N-10 nucleotide;
(6) a passenger strand where one or more Rp, Sp, or stereorandom non-negatively charged internucleotidic linkage occurs upstream, i.e., in the 5′ direction, relative to the central nucleotide of the passenger strand;
(7) a passenger strand where one or more Rp, Sp, or stereorandom non-negatively charged internucleotidic linkage occurs downstream, i.e., in the 3′ direction, relative to the central nucleotide of the passenger strand;
(8) a passenger strand where one or more backbone phosphorothioate chiral centers in Rp or Sp configuration internucleotidic linkage occurs upstream, i.e., in the 5′ direction, relative to the central nucleotide of the passenger strand;
(9) a passenger strand where one or more backbone phosphorothioate chiral centers in Rp or Sp configuration internucleotidic linkage internucleotidic linkage occurs downstream, i.e., in the 3′ direction, relative to the central nucleotide of the passenger strand; and
(10) a passenger strand comprising one or more modified sugars, e.g. Homo-DNA, between the 5′ terminal (+1) nucleotide and the penultimate (N-1) nucleotide.

In certain embodiments, the ds oligonucleotide further comprises a 2′ modification, e.g., a 2′ F modification, of the 3′ nucleotide of a nucleotide pair linked by an Rp, Sp, or stereorandom non-negatively charged internucleotidic linkage, and the passenger strand comprises one or more backbone chiral centers in Rp or Sp configuration. In certain embodiments, the one or more Rp, Sp, or stereorandom non-negatively charged internucleotidic linkage incorporated into the guide or passenger strand is an Rp non-negatively charged internucleotidic linkage. In certain embodiments, the one or more Rp, Sp, or stereorandom non-negatively charged internucleotidic linkage is an Sp non-negatively charged internucleotidic linkage. In certain embodiments, the one or more Rp, Sp, or stereorandom non-negatively charged internucleotidic linkage is a stereorandom non-negatively charged internucleotidic linkage. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, further comprise one or a plurality of modified sugars. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the 5′ terminal (+1) nucleotide, second (+2) nucleotide, third (+3) nucleotide, fourth (+4) nucleotide, fifth (+5) nucleotide, sixth (+6) nucleotide, seventh (+7) nucleotide, eighth (+8) nucleotide, ninth (+9) nucleotide, tenth (+10) nucleotide, twelfth (+12) nucleotide, sixteenth (+16) nucleotide, seventeenth (+17) nucleotide, eighteenth (+18) nucleotide, nineteenth (+19) nucleotide, twentieth (+20) nucleotide, twenty-first (+21) nucleotide, twenty-second (+22), and/or twenty-third (+23), the 3′ terminal, nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the 5′ terminal (+1) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the second (+2) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the third (+3) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the fourth (+4) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the fifth (+5) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the sixth (+6) nucleotide. In certain embodiments, the modified sugar comprises a 2′-F modification, 2′-H modification, 2′-OH modification, 2′-O-alkyl modification, e.g., 2′-O— methyl (OMe) modification, 2′-methoxyethyl (MOE) modification, 5′-alkyl modification, e.g., 5′-(R)-methyl or 5′-(S)-methyl, DNA, locked nucleic acid (LNA), unlocked nucleic acid (UNA), glycol nucleic acid (GNA), or Homo-DNA. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the 5′ terminal (+1) nucleotide, second (+2) nucleotide, third (+3) nucleotide, fourth (+4) nucleotide, fifth (+5) nucleotide, and/or sixth (+6) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the 5′ terminal (+1) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the second (+2) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the third (+3) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the fourth (+4) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the fifth (+5) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the sixth (+6) nucleotide.

In certain embodiments, the ds oligonucleotide comprises a guide strand backbone phosphoryl guanidine chiral center in the Sp configuration between the +3 nucleotide and the immediately downstream (+4) nucleotide, i.e., in the 3′ direction, and the guide strand further comprises one or more Rp, Sp, or stereorandom non-negatively charged internucleotidic linkage occurs between any two adjacent nucleotides between the second (+2) nucleotide relative to the 5′ terminal nucleotide of the guide strand and the penultimate 3′ (N-1) nucleotide of the guide strand, where N is the 3′ terminal nucleotide, a 2′ modification, e.g., a 2′ F modification, of the 3′ nucleotide of a nucleotide pair linked by an Rp, Sp, or stereorandom non-negatively charged internucleotidic linkage, and the passenger strand comprises one or more backbone chiral centers in Rp or Sp configuration. In certain embodiments, the one or more Rp, Sp, or stereorandom non-negatively charged internucleotidic linkage incorporated into the guide strand is an Rp non-negatively charged internucleotidic linkage. In certain embodiments, the one or more Rp, Sp, or stereorandom non-negatively charged internucleotidic linkage is an Sp non-negatively charged internucleotidic linkage. In certain embodiments, the one or more Rp, Sp, or stereorandom non-negatively charged internucleotidic linkage is a stereorandom non-negatively charged internucleotidic linkage. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, further comprise one or a plurality of modified sugars. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the 5′ terminal (+1) nucleotide, second (+2) nucleotide, third (+3) nucleotide, fourth (+4) nucleotide, fifth (+5) nucleotide, sixth (+6) nucleotide, seventh (+7) nucleotide, eighth (+8) nucleotide, ninth (+9) nucleotide, tenth (+10) nucleotide, twelfth (+12) nucleotide, sixteenth (+16) nucleotide, seventeenth (+17) nucleotide, eighteenth (+18) nucleotide, nineteenth (+19) nucleotide, twentieth (+20) nucleotide, twenty-first (+21) nucleotide, twenty-second (+22), and/or twenty-third (+23), the 3′ terminal, nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the 5′ terminal (+1) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the second (+2) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the third (+3) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the fourth (+4) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the fifth (+5) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the sixth (+6) nucleotide. In certain embodiments, the modified sugar comprises a 2′-F modification, 2′-H modification, 2′-OH modification, 2′-O-alkyl modification, e.g., 2′-O— methyl (OMe) modification, 2′-methoxyethyl (MOE) modification, 5′-alkyl modification, e.g., 5′-(R)-methyl or 5′-(S)-methyl, DNA, locked nucleic acid (LNA), unlocked nucleic acid (UNA), glycol nucleic acid (GNA), or Homo-DNA. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the 5′ terminal (+1) nucleotide, second (+2) nucleotide, third (+3) nucleotide, fourth (+4) nucleotide, fifth (+5) nucleotide, and/or sixth (+6) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the 5′ terminal (+1) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the second (+2) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the third (+3) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the fourth (+4) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the fifth (+5) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the sixth (+6) nucleotide.

In certain embodiments, the ds oligonucleotide comprises a guide strand backbone phosphoryl guanidine chiral center in the Sp configuration between the +3 nucleotide and the immediately downstream (+4) nucleotide, i.e., in the 3′ direction, and the guide strand further comprises backbone phosphorothioate chiral centers in Sp configuration between the 3′ terminal nucleotide and the penultimate (N-1) nucleotide and as between the penultimate (N-1) nucleotide and the immediately upstream (N-2) nucleotide, a 2′ modification, e.g., a 2′ F modification, of the 3′ nucleotide of a nucleotide pair linked by an Rp, Sp, or stereorandom non-negatively charged internucleotidic linkage, and the passenger strand comprises 0-n Rp, Sp, or stereorandom non-negatively charged internucleotidic linkages, where n is about 1 to 49 and one or more backbone chiral centers in Rp or Sp configuration.

In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, further comprise one or a plurality of modified sugars. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the 5′ terminal (+1) nucleotide, second (+2) nucleotide, third (+3) nucleotide, fourth (+4) nucleotide, fifth (+5) nucleotide, sixth (+6) nucleotide, seventh (+7) nucleotide, eighth (+8) nucleotide, ninth (+9) nucleotide, tenth (+10) nucleotide, twelfth (+12) nucleotide, sixteenth (+16) nucleotide, seventeenth (+17) nucleotide, eighteenth (+18) nucleotide, nineteenth (+19) nucleotide, twentieth (+20) nucleotide, twenty-first (+21) nucleotide, twenty-second (+22), and/or twenty-third (+23), the 3′ terminal, nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the 5′ terminal (+1) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the second (+2) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the third (+3) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the fourth (+4) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the fifth (+5) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the sixth (+6) nucleotide. In certain embodiments, the modified sugar comprises a 2′-F modification, 2′-H modification, 2′-OH modification, 2′-O-alkyl modification, e.g., 2′-O— methyl (OMe) modification, 2′-methoxyethyl (MOE) modification, 5′-alkyl modification, e.g., 5′-(R)-methyl or 5′-(S)-methyl, DNA, locked nucleic acid (LNA), unlocked nucleic acid (UNA), glycol nucleic acid (GNA), or Homo-DNA. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the 5′ terminal (+1) nucleotide, second (+2) nucleotide, third (+3) nucleotide, fourth (+4) nucleotide, fifth (+5) nucleotide, and/or sixth (+6) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the 5′ terminal (+1) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the second (+2) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the third (+3) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the fourth (+4) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the fifth (+5) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the sixth (+6) nucleotide.

In certain embodiments, the one or more Rp, Sp, or stereorandom non-negatively charged internucleotidic linkage incorporated into the guide strand is an Rp non-negatively charged internucleotidic linkage. In certain embodiments, the one or more Rp, Sp, or stereorandom non-negatively charged internucleotidic linkage is an Sp non-negatively charged internucleotidic linkage. In certain embodiments, the one or more Rp, Sp, or stereorandom non-negatively charged internucleotidic linkage is a stereorandom non-negatively charged internucleotidic linkage. In certain further embodiments, the passenger strand comprises an Sp backbone phosphorothioate chiral center between the 5′ terminal (+1) nucleotide and the immediately downstream (+2) nucleotide and an Sp backbone phosphorothioate chiral center between the penultimate (N-1) nucleotide and the 3′ terminal (N) nucleotide.

In certain embodiments, the ds oligonucleotide comprises a guide strand backbone phosphoryl guanidine chiral center in the Sp configuration between the +3 nucleotide and the immediately downstream (+4) nucleotide, i.e., in the 3′ direction, and the guide strand further comprises backbone phosphorothioate chiral centers in Rp, Sp, or alternating configurations between the 5′ terminal (+1) nucleotide and the immediately downstream (+2) nucleotide and between the +2 nucleotide and the immediately downstream (+3) nucleotide, a 2′ modification, e.g., a 2′ F modification, of the 3′ nucleotide of a nucleotide pair linked by an Rp, Sp, or stereorandom non-negatively charged internucleotidic linkage, and the passenger strand comprises 0-n Rp, Sp, or stereorandom non-negatively charged internucleotidic linkages, where n is about 1 to 49 and one or more backbone chiral centers in Rp or Sp configuration.

In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, further comprise one or a plurality of modified sugars. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the 5′ terminal (+1) nucleotide, second (+2) nucleotide, third (+3) nucleotide, fourth (+4) nucleotide, fifth (+5) nucleotide, sixth (+6) nucleotide, seventh (+7) nucleotide, eighth (+8) nucleotide, ninth (+9) nucleotide, tenth (+10) nucleotide, twelfth (+12) nucleotide, sixteenth (+16) nucleotide, seventeenth (+17) nucleotide, eighteenth (+18) nucleotide, nineteenth (+19) nucleotide, twentieth (+20) nucleotide, twenty-first (+21) nucleotide, twenty-second (+22), and/or twenty-third (+23), the 3′ terminal, nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the 5′ terminal (+1) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the second (+2) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the third (+3) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the fourth (+4) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the fifth (+5) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the sixth (+6) nucleotide. In certain embodiments, the modified sugar comprises a 2′-F modification, 2′-H modification, 2′-OH modification, 2′-O-alkyl modification, e.g., 2′-O— methyl (OMe) modification, 2′-methoxyethyl (MOE) modification, 5′-alkyl modification, e.g., 5′-(R)-methyl or 5′-(S)-methyl, DNA, locked nucleic acid (LNA), unlocked nucleic acid (UNA), glycol nucleic acid (GNA), or Homo-DNA. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the 5′ terminal (+1) nucleotide, second (+2) nucleotide, third (+3) nucleotide, fourth (+4) nucleotide, fifth (+5) nucleotide, and/or sixth (+6) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the 5′ terminal (+1) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the second (+2) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the third (+3) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the fourth (+4) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the fifth (+5) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the sixth (+6) nucleotide.

In certain embodiments, the one or more Rp, Sp, or stereorandom non-negatively charged internucleotidic linkage incorporated into the guide strand is an Rp non-negatively charged internucleotidic linkage. In certain embodiments, the one or more Rp, Sp, or stereorandom non-negatively charged internucleotidic linkage is an Sp non-negatively charged internucleotidic linkage. In certain embodiments, the one or more Rp, Sp, or stereorandom non-negatively charged internucleotidic linkage is a stereorandom non-negatively charged internucleotidic linkage. In certain further embodiments, the passenger strand comprises an Sp backbone phosphorothioate chiral center between the 5′ terminal (+1) nucleotide and the immediately downstream (+2) nucleotide and an Sp backbone phosphorothioate chiral center between the penultimate (N-1) nucleotide and the 3′ terminal (N) nucleotide.

In certain embodiments, the ds oligonucleotide comprises a guide strand backbone phosphoryl guanidine chiral center in the Sp configuration between the +3 nucleotide and the immediately downstream (+4) nucleotide, i.e., in the 3′ direction, and the guide strand further comprises one or more backbone phosphorothioate chiral centers in Rp or Sp configuration upstream of backbone phosphorothioate chiral centers in Sp configuration between the 3′ terminal nucleotide and the penultimate (N-1) nucleotide and as between the penultimate (N-1) nucleotide and the immediately upstream (N-2) nucleotide, a 2′ modification, e.g., a 2′ F modification, of the 3′ nucleotide of a nucleotide pair linked by an Rp, Sp, or stereorandom non-negatively charged internucleotidic linkage, and the passenger strand comprises 0-n Rp, Sp, or stereorandom non-negatively charged internucleotidic linkages, where n is about 1 to 49 and one or more backbone chiral centers in Rp or Sp configuration.

In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, further comprise one or a plurality of modified sugars. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the 5′ terminal (+1) nucleotide, second (+2) nucleotide, third (+3) nucleotide, fourth (+4) nucleotide, fifth (+5) nucleotide, sixth (+6) nucleotide, seventh (+7) nucleotide, eighth (+8) nucleotide, ninth (+9) nucleotide, tenth (+10) nucleotide, twelfth (+12) nucleotide, sixteenth (+16) nucleotide, seventeenth (+17) nucleotide, eighteenth (+18) nucleotide, nineteenth (+19) nucleotide, twentieth (+20) nucleotide, twenty-first (+21) nucleotide, twenty-second (+22), and/or twenty-third (+23), the 3′ terminal, nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the 5′ terminal (+1) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the second (+2) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the third (+3) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the fourth (+4) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the fifth (+5) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the sixth (+6) nucleotide. In certain embodiments, the modified sugar comprises a 2′-F modification, 2′-H modification, 2′-OH modification, 2′-O-alkyl modification, e.g., 2′-O— methyl (OMe) modification, 2′-methoxyethyl (MOE) modification, 5′-alkyl modification, e.g., 5′-(R)-methyl or 5′-(S)-methyl, DNA, locked nucleic acid (LNA), unlocked nucleic acid (UNA), glycol nucleic acid (GNA), or Homo-DNA. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the 5′ terminal (+1) nucleotide, second (+2) nucleotide, third (+3) nucleotide, fourth (+4) nucleotide, fifth (+5) nucleotide, and/or sixth (+6) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the 5′ terminal (+1) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the second (+2) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the third (+3) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the fourth (+4) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the fifth (+5) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the sixth (+6) nucleotide.

In certain embodiments, the one or more Rp, Sp, or stereorandom non-negatively charged internucleotidic linkage incorporated into the guide strand is an Rp non-negatively charged internucleotidic linkage. In certain embodiments, the one or more Rp, Sp, or stereorandom non-negatively charged internucleotidic linkage is an Sp non-negatively charged internucleotidic linkage. In certain embodiments, the one or more Rp, Sp, or stereorandom non-negatively charged internucleotidic linkage is a stereorandom non-negatively charged internucleotidic linkage. In certain further embodiments, the passenger strand comprises an Sp backbone phosphorothioate chiral center between the 5′ terminal (+1) nucleotide and the immediately downstream (+2) nucleotide and an Sp backbone phosphorothioate chiral center between the penultimate (N-1) nucleotide and the 3′ terminal (N) nucleotide.

In certain embodiments, the one or more Rp, Sp, or stereorandom non-negatively charged internucleotidic linkage incorporated into the guide strand is an Rp non-negatively charged internucleotidic linkage. In certain embodiments, the one or more Rp, Sp, or stereorandom non-negatively charged internucleotidic linkage is an Sp non-negatively charged internucleotidic linkage. In certain embodiments, the one or more Rp, Sp, or stereorandom non-negatively charged internucleotidic linkage is a stereorandom non-negatively charged internucleotidic linkage. In certain further embodiments, the passenger strand comprises an Rp backbone phosphorothioate chiral center between the 5′ terminal (+1) nucleotide and the immediately downstream (+2) nucleotide and an Rp backbone phosphorothioate chiral center between the penultimate (N-1) nucleotide and the 3′ terminal (N) nucleotide.

In certain embodiments, the one or more Rp, Sp, or stereorandom non-negatively charged internucleotidic linkage incorporated into the guide strand is an Rp non-negatively charged internucleotidic linkage. In certain embodiments, the one or more Rp, Sp, or stereorandom non-negatively charged internucleotidic linkage is an Sp non-negatively charged internucleotidic linkage. In certain embodiments, the one or more Rp, Sp, or stereorandom non-negatively charged internucleotidic linkage is a stereorandom non-negatively charged internucleotidic linkage. In certain further embodiments, the passenger strand comprises an Rp backbone phosphorothioate chiral center between the 5′ terminal (+1) nucleotide and the immediately downstream (+2) nucleotide and an Sp backbone phosphorothioate chiral center between the penultimate (N-1) nucleotide and the 3′ terminal (N) nucleotide.

In certain embodiments, the ds oligonucleotide comprises a guide strand backbone phosphoryl guanidine chiral center in the Sp configuration between the +3 nucleotide and the immediately downstream (+4) nucleotide, i.e., in the 3′ direction, and the guide strand further comprises one or more Rp, Sp, or stereorandom non-negatively charged internucleotidic linkage occurs between any two adjacent nucleotides between the second (+2) nucleotide relative to the 5′ terminal nucleotide of the guide strand and the penultimate 3′ (N-1) nucleotide of the guide strand, where N is the 3′ terminal nucleotide, a 2′ modification, e.g., a 2′ F modification, of the 3′ nucleotide of a nucleotide pair linked by an Rp, Sp, or stereorandom non-negatively charged internucleotidic linkage, and the passenger strand comprises 0-n Rp, Sp, or stereorandom non-negatively charged internucleotidic linkages, where n is about 1 to 49 and one or more backbone chiral centers in Rp or Sp configuration.

In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, further comprise one or a plurality of modified sugars. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the 5′ terminal (+1) nucleotide, second (+2) nucleotide, third (+3) nucleotide, fourth (+4) nucleotide, fifth (+5) nucleotide, sixth (+6) nucleotide, seventh (+7) nucleotide, eighth (+8) nucleotide, ninth (+9) nucleotide, tenth (+10) nucleotide, twelfth (+12) nucleotide, sixteenth (+16) nucleotide, seventeenth (+17) nucleotide, eighteenth (+18) nucleotide, nineteenth (+19) nucleotide, twentieth (+20) nucleotide, twenty-first (+21) nucleotide, twenty-second (+22), and/or twenty-third (+23), the 3′ terminal, nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the 5′ terminal (+1) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the second (+2) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the third (+3) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the fourth (+4) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the fifth (+5) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a modified sugar at the sixth (+6) nucleotide. In certain embodiments, the modified sugar comprises a 2′-F modification, 2′-H modification, 2′-OH modification, 2′-O-alkyl modification, e.g., 2′-O— methyl (OMe) modification, 2′-methoxyethyl (MOE) modification, 5′-alkyl modification, e.g., 5′-(R)-methyl or 5′-(S)-methyl, DNA, locked nucleic acid (LNA), unlocked nucleic acid (UNA), glycol nucleic acid (GNA), or Homo-DNA. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the 5′ terminal (+1) nucleotide, second (+2) nucleotide, third (+3) nucleotide, fourth (+4) nucleotide, fifth (+5) nucleotide, and/or sixth (+6) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the 5′ terminal (+1) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the second (+2) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the third (+3) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the fourth (+4) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the fifth (+5) nucleotide. In certain embodiments, the ds oligonucleotides comprising a guide strand backbone phosphoryl guanidine chiral center in the Sp configuration between the +3 nucleotide and the immediately downstream (+4) nucleotide, i.e., in the 3′ direction, comprise a 2′-F modification of the sixth (+6) nucleotide.

In certain embodiments, the one or more Rp, Sp, or stereorandom non-negatively charged internucleotidic linkage incorporated into the guide strand is an Rp non-negatively charged internucleotidic linkage. In certain embodiments, the one or more Rp, Sp, or stereorandom non-negatively charged internucleotidic linkage is an Sp non-negatively charged internucleotidic linkage. In certain embodiments, the one or more Rp, Sp, or stereorandom non-negatively charged internucleotidic linkage is a stereorandom non-negatively charged internucleotidic linkage. In certain further embodiments, the passenger strand comprises an Sp backbone phosphorothioate chiral center between the 5′ terminal (+1) nucleotide and the immediately downstream (+2) nucleotide and an Sp backbone phosphorothioate chiral center between the penultimate (N-1) nucleotide and the 3′ terminal (N) nucleotide. In certain further embodiments, the passenger strand comprises an Rp backbone phosphorothioate chiral center between the 5′ terminal (+1) nucleotide and the immediately downstream (+2) nucleotide and an Sp backbone phosphorothioate chiral center between the penultimate (N-1) nucleotide and the 3′ terminal (N) nucleotide. In certain embodiments, a RNAi oligonucleotide comprises a sequence that is completely or substantially identical to or is completely or substantially complementary to 10 or more (e.g., 10, 11, 12, 13, 14, 15, 16, 17, 18, 19, 20 or more) contiguous bases of a target genomic sequence or a transcript therefrom (e.g., mRNA (e.g., pre-mRNA, mRNA after splicing, etc.)). In certain embodiments, a RNAi oligonucleotide comprises a sequence that is completely complementary to 10 or more (e.g., 10, 11, 12, 13, 14, 15, 16, 17, 18, 19, 20 or more) contiguous bases of a target transcript. In certain embodiments, the number of contiguous bases is about 15-20. In certain embodiments, the number of contiguous bases is about 20. In certain embodiments, an RNAi oligonucleotide that can hybridize with a target transcript (e.g., pre-mRNA, RNA, etc.) and can reduce the level of the target transcript and/or a protein encoded by the target transcript.

In certain embodiments, the present disclosure provides a dsRNAi oligonucleotide as disclosed herein, e.g., in Table 1. In certain embodiments, the present disclosure provides a dsRNAi oligonucleotide having a base sequence disclosed herein, e.g., in Table 1, or a portion thereof comprising at least 10 (e.g., 10, 11, 12, 13, 14, 15, 16, 17, 18, 19, 20 or more) contiguous bases, wherein the RNAi oligonucleotide is stereorandom or not chirally controlled, and wherein each T can be independently substituted with U and vice versa.

In certain embodiments, internucleotidic linkages of an oligonucleotide comprise or consist of 1-5, 1-10, 1-15, 1-20, 1-25, 1-30, 1-40, 1-50, or 1, 2, 3, 4, 5, 6, 7, 8, 9, 10, 11, 12, 13, 14, 15, 16, 17, 18, 19, 20 or more chirally controlled internucleotidic linkages. In certain embodiments, the present disclosure provides a dsRNAi oligonucleotide composition wherein the dsRNAi oligonucleotides comprise at least one chirally controlled internucleotidic linkage. In certain embodiments, the present disclosure provides a dsRNAi oligonucleotide composition wherein the dsRNAi oligonucleotides are stereorandom or not chirally controlled. In certain embodiments, in a dsRNAi oligonucleotide, at least one internucleotidic linkage is stereorandom and at least one internucleotidic linkage is chirally controlled.

In certain embodiments, internucleotidic linkages of an oligonucleotide comprise or consist of one or more neutrally charged internucleotidic linkages.

### 1.1 Double Stranded Oligonucleotides

In certain embodiments, the present disclosure provides oligonucleotides of various designs, which may comprise various nucleobases and patterns thereof, sugars and patterns thereof, internucleotidic linkages and patterns thereof, and/or additional chemical moieties and patterns thereof as described in the present disclosure. In certain embodiments, provided dsRNAi oligonucleotides can direct a decrease in the expression, level and/or activity of a gene and/or one or more of its products (e.g., transcripts, mRNA, proteins, etc.). In certain embodiments, provided dsRNAi oligonucleotides can direct a decrease in the expression, level and/or activity of a gene and/or one or more of its products in a cell of a subject or patient. In certain embodiments, a cell normally expresses or produces a protein. In certain embodiments, provided dsRNAi oligonucleotides can direct a decrease in the expression, level and/or activity of a target gene or a gene product and has a base sequence which consists of, comprises, or comprises a portion (e.g., a span of 1-5, 1-10, 1-15, 1-20, 1-25, 1-30, 1-40, 1-50, or 1, 2, 3, 4, 5, 6, 7, 8, 9, 10, 11, 12, 13, 14, 15, 16, 17, 18, 19, 20 or more contiguous bases) of the base sequence of a dsRNAi oligonucleotide disclosed herein, wherein each T can be independently substituted with U and vice versa, and the ds oligonucleotide comprises at least one non-naturally-occurring modification of abase, sugar and/or internucleotidic linkage.

In certain embodiments, dsRNAi oligonucleotides can direct a decrease in the expression, level and/or activity of a target gene, e.g., a target gene, or a product thereof. In certain embodiments, provided ds oligonucleotides can direct a decrease in the expression and/or level of a target gene or its gene product. In certain embodiments, provided ds oligonucleotides can direct a decrease in levels of target products. In certain embodiments, provided ds oligonucleotide can reduce levels of transcripts of target genes. In certain embodiments, provided ds oligonucleotide can reduce levels of mRNA of target genes. In certain embodiments, provided ds oligonucleotide can reduce levels of proteins encoded by target genes. In certain embodiments, provided ds oligonucleotides can direct a decrease in the expression and/or level of a target gene or its gene product via RNA interference. In certain embodiments, provided ds oligonucleotides can direct a decrease in the expression and/or level of a target gene or its gene product via a biochemical mechanism which does not involve RNA interference or RISC (including, but not limited to, RNaseH-mediated knockdown or steric hindrance of gene expression). In certain embodiments, provided ds oligonucleotides can direct a decrease in the expression and/or level of a target gene or its gene product via RNA interference and/or RNase H-mediated knockdown. In certain embodiments, provided ds oligonucleotides can direct a decrease in the expression and/or level of a target gene or its gene product by sterically blocking translation after binding to a target gene mRNA, and/or by altering or interfering with mRNA splicing and/or exon inclusion or exclusion. In certain embodiments, provided ds oligonucleotides comprise one or more structural elements described herein or known in the art in accordance with the present disclosure, e.g., base sequences; modifications; stereochemistry; patterns of internucleotidic linkages; GC contents; long GC stretches; patterns of backbone linkages; patterns of backbone chiral centers; patterns of backbone phosphorus modifications; additional chemical moieties, including but not limited to, one or more targeting moieties, lipid moieties, and/or carbohydrate moieties, etc.; seed regions; post-seed regions; 5′-end structures; 5′-end regions; 5′ nucleotide moieties; 3′-end regions; 3′-terminal dinucleotides; 3′-end caps; etc. In certain embodiments, a seed region of an oligonucleotide is or comprises the second to eighth, second to seventh, second to sixth, third to eighth, third to seventh, third to seven, or fourth to eighth or fourth to seventh nucleotides, counting from the 5′ end; and the post-seed region of the oligonucleotide is the region immediately 3′ to the seed region, and interposed between the seed region and the 3′ end region. In certain embodiments, a provided composition comprises a ds oligonucleotide. In certain embodiments, a provided composition comprises one or more lipid moieties, one or more carbohydrate moieties (unless otherwise specified, other than sugar moieties of nucleoside units that form oligonucleotide chain with internucleotidic linkages), and/or one or more targeting components. In certain embodiments, ds RNAi oligonucleotides can direct a decrease in the expression, level and/or activity of a target gene or a product thereof by sterically blocking translation after binding to a target gene mRNA, and/or by altering or interfering with mRNA splicing. Regardless, however, the present disclosure is not limited to any particular mechanism. In certain embodiments, the present disclosure provides ds oligonucleotides, compositions, methods, etc., capable of operating via double-stranded RNA interference, single-stranded RNA interference, RNase H-mediated knock-down, steric hindrance of translation, or a combination of two or more such mechanisms.

In certain embodiments, a dsRNAi oligonucleotide comprises a structural element or a portion thereof described herein, e.g., in Table 1. In certain embodiments, a dsRNAi oligonucleotide comprises a base sequence (or a portion thereof) described herein, wherein each T can be independently substituted with U and vice versa, a chemical modification or a pattern of chemical modifications (or a portion thereof), and/or a format or a portion thereof described herein. In certain embodiments, a dsRNAi oligonucleotide has a base sequence which comprises the base sequence (or a portion thereof) wherein each T can be independently substituted with U, pattern of chemical modifications (or a portion thereof), and/or a format of an oligonucleotide disclosed herein, e.g., in Table 1, or otherwise disclosed herein. In certain embodiments, such ds oligonucleotides, e.g., dsRNAi oligonucleotides reduce expression, level and/or activity of a gene, e.g., a gene, or a gene product thereof.

Among other things, dsRNAi oligonucleotides may hybridize to their target nucleic acids (e.g., pre-mRNA, mature mRNA, etc.). For example, in certain embodiments, a dsRNAi oligonucleotide can hybridize to a nucleic acid derived from a DNA strand (either strand of the gene). In certain embodiments, a dsRNAi oligonucleotide can hybridize to a transcript. In certain embodiments, a dsRNAi oligonucleotide can hybridize to a target nucleic acid in any stage of RNA processing, including but not limited to a pre-mRNA or a mature mRNA. In certain embodiments, a dsRNAi oligonucleotide can hybridize to any element of a target nucleic acid or its complement, including but not limited to: a promoter region, an enhancer region, a transcriptional stop region, a translational start signal, a translation stop signal, a coding region, a non-coding region, an exon, an intron, an intron/exon or exon/intron junction, the 5′ UTR, or the 3′ UTR. In certain embodiments, dsRNAi oligonucleotides can hybridize to their targets with no more than 2 mismatches. In certain embodiments, dsRNAi oligonucleotides can hybridize to their targets with no more than one mismatch. In certain embodiments, dsRNAi oligonucleotides can hybridize to their targets with no mismatches (e.g., when all C-G and/or A-T/U base paring).

In certain embodiments, a ds oligonucleotide can hybridize to two or more variants of transcripts. In certain embodiments, a dsRNAi oligonucleotide can hybridize to two or more or all variants of a transcript. In certain embodiments, a dsRNAi oligonucleotide can hybridize to two or more or all variants of a transcript derived from the sense strand.

In certain embodiments, a target of a dsRNAi oligonucleotide is an RNA which is not a mRNA.

In certain embodiments, ds oligonucleotides, e.g., dsRNAi oligonucleotides, contain increased levels of one or more isotopes. In certain embodiments, ds oligonucleotides, e.g., dsRNAi oligonucleotides, are labeled, e.g., by one or more isotopes of one or more elements, e.g., hydrogen, carbon, nitrogen, etc. In certain embodiments, ds oligonucleotides, e.g., dsRNAi oligonucleotides, in provided compositions, e.g., ds oligonucleotides of a plurality of a composition, comprise base modifications, sugar modifications, and/or internucleotidic linkage modifications, wherein the ds oligonucleotides contain an enriched level of deuterium. In certain embodiments, oligonucleotides, e.g., RNAi oligonucleotides, are labeled with deuterium (replacing −¹H with −²H) at one or more positions. In certain embodiments, one or more ¹H of a ds oligonucleotide chain or any moiety conjugated to the ds oligonucleotide chain (e.g., a targeting moiety, etc.) is substituted with ²H. Such ds oligonucleotides can be used in compositions and methods described herein.

In certain embodiments, the present disclosure provides a ds oligonucleotide composition comprising a plurality of ds oligonucleotides which:
1) have a common base sequence complementary to atarget sequence (e.g., a target sequence) in a transcript; and
2) comprise one or more modified sugar moieties and/or modified internucleotidic linkages.

In certain embodiments, dsRNAi oligonucleotides having a common base sequence may have the same pattern of nucleoside modifications, e.g., sugar modifications, base modifications, etc. In certain embodiments, a pattern of nucleoside modifications may be represented by a combination of locations and modifications. In certain embodiments, a pattern of backbone linkages comprises locations and types (e.g., phosphate, phosphorothioate, substituted phosphorothioate, etc.) of each internucleotidic linkage.

In certain embodiments, ds oligonucleotides of a plurality, e.g., in provided compositions, are of the same ds oligonucleotide type. In certain embodiments, ds oligonucleotides of an ds oligonucleotide type have a common pattern of sugar modifications. In certain embodiments, ds oligonucleotides of a ds oligonucleotide type have a common pattern of base modifications. In certain embodiments, ds oligonucleotides of a ds oligonucleotide type have a common pattern of nucleoside modifications. In certain embodiments, ds oligonucleotides of a ds oligonucleotide type have the same constitution. In certain embodiments, ds oligonucleotides of a ds oligonucleotide type are identical. In certain embodiments, ds oligonucleotides of a plurality are identical. In certain embodiments, ds oligonucleotides of a plurality share the same constitution.

In certain embodiments, as exemplified herein, dsRNAi oligonucleotides are chiral controlled, comprising one or more chirally controlled internucleotidic linkages. In certain embodiments, ds RNAi oligonucleotides are stereochemically pure. In certain embodiments, dsRNAi oligonucleotides are substantially separated from other stereoisomers.

In certain embodiments, RNAi oligonucleotides comprise one or more modified nucleobases, one or more modified sugars, and/or one or more modified internucleotidic linkages.

In certain embodiments, dsRNAi oligonucleotides comprise one or more modified sugars. In certain embodiments, ds oligonucleotides of the present disclosure comprise one or more modified nucleobases. Various modifications can be introduced to a sugar and/or nucleobase in accordance with the present disclosure. For example, in certain embodiments, a modification is a modification described in U.S. Pat. No. 9,006,198. In certain embodiments, a modification is a modification described in U.S. Pat. Nos. 9,394,333, 9,744,183, 9,605,019, 9,598,458, 9,982,257, U.S. Ser. No. 10/160,969, U.S. Ser. No. 10/479,995, US 2020/0056173, US 2018/0216107, US 2019/0127733, U.S. Ser. No. 10/450,568, US 2019/0077817, US 2019/0249173, US 2019/0375774, WO 2018/223056, WO 2018/223073, WO 2018/223081, WO 2018/237194, WO 2019/032607, WO 2019/055951, WO 2019/075357, WO 2019/200185, WO 2019/217784, and/or WO 2019/032612, the sugar, base, and internucleotidic linkage modifications of each of which are independently incorporated herein by reference.

As used in the present disclosure, in certain embodiments, “one or more” is 1-200, 1-150, 1-100, 1-90, 1-80, 1-70, 1-60, 1-50, 1-40, 1-30, or 1, 2, 3, 4, 5, 6, 7, 8, 9, 10, 11, 12, 13, 14, 15, 16, 17, 18, 19, 20, 21, 22, 23, 24, or 25. In certain embodiments, “one or more” is one. In certain embodiments, “one or more” is two. In certain embodiments, “one or more” is three. In certain embodiments, “one or more” is four. In certain embodiments, “one or more” is five. In certain embodiments, “one or more” is six. In certain embodiments, “one or more” is seven. In certain embodiments, “one or more” is eight. In certain embodiments, “one or more” is nine. In certain embodiments, “one or more” is ten. In certain embodiments, “one or more” is at least one. In certain embodiments, “one or more” is at least two. In certain embodiments, “one or more” is at least three. In certain embodiments, “one or more” is at least four. In certain embodiments, “one or more” is at least five. In certain embodiments, “one or more” is at least six. In certain embodiments, “one or more” is at least seven. In certain embodiments, “one or more” is at least eight. In certain embodiments, “one or more” is at least nine. In certain embodiments, “one or more” is at least ten.

As used in the present disclosure, in certain embodiments, “at least one” is 1-200, 1-150, 1-100, 1-90, 1-80, 1-70, 1-60, 1-50, 1-40, 1-30, or 1, 2, 3, 4, 5, 6, 7, 8, 9, 10, 11, 12, 13, 14, 15, 16, 17, 18, 19, 20, 21, 22, 23, 24, or 25. In certain embodiments, “at least one” is one. In certain embodiments, “at least one” is two. In certain embodiments, “at least one” is three. In certain embodiments, “at least one” is four. In certain embodiments, “at least one” is five. In certain embodiments, “at least one” is six. In certain embodiments, “at least one” is seven. In certain embodiments, “at least one” is eight. In certain embodiments, “at least one” is nine. In certain embodiments, “at least one” is ten.

In certain embodiments, a dsRNAi oligonucleotide is or comprises a dsRNAi oligonucleotide described in Table 1.

As demonstrated in the present disclosure, in certain embodiments, a provided ds oligonucleotide (e.g., a dsRNAi oligonucleotide) is characterized in that, when it is contacted with the transcript in a knockdown system, knockdown of its target (e.g., a transcript for a target oligonucleotide).

In certain embodiments, ds oligonucleotides are provided as salt forms. In certain embodiments, ds oligonucleotides are provided as salts comprising negatively-charged internucleotidic linkages (e.g., phosphorothioate internucleotidic linkages, natural phosphate linkages, etc.) existing as their salt forms. In certain embodiments, ds oligonucleotides are provided as pharmaceutically acceptable salts. In certain embodiments, ds oligonucleotides are provided as metal salts. In certain embodiments, ds oligonucleotides are provided as sodium salts. In certain embodiments, ds oligonucleotides are provided as metal salts, e.g., sodium salts, wherein each negatively-charged internucleotidic linkage is independently in a salt form (e.g., for sodium salts, —O—P(O)(SNa)—O— for a phosphorothioate internucleotidic linkage, —O—P(O)(ONa)—O— for a natural phosphate linkage, etc.).

### 1.2 Regions of Double Stranded Oligonucleotides

### 1.2.1 Base Sequences

In certain embodiments, a dsRNAi oligonucleotide comprises a base sequence described herein or a portion (e.g., a span of 5-50, 5-40, 5-30, 5-20, or 5, 6, 7, 8, 9, 10, 11, 12, 13, 14, 15, 16, 17, 18, 19, 20, 21, 22, 23, 24, 25, 26, 27, 28, 20 or at least 10, at least 15, contiguous nucleobases) thereof with 0-5 (e.g., 0, 1, 2, 3, 4 or 5) mismatches, wherein each T can be independently substituted with U and vice versa. In certain embodiments, a dsRNAi oligonucleotide comprises a base sequence described herein, or a portion thereof, wherein a portion is a span of at least 10 contiguous nucleobases, or a span of at least 15 contiguous nucleobases with 1-5 mismatches. In certain embodiments, dsRNAi oligonucleotides comprise a base sequence described herein, or a portion thereof, wherein a portion is a span of at least 10 contiguous nucleobases, or a span of at least 10 contiguous nucleobases with 1-5 mismatches, wherein each T can be independently substituted with U and vice versa. In certain embodiments, base sequences of ds oligonucleotides comprise or consists of 10-50 (e.g., about or at least 10, 11, 12, 13, 14, 15, 16, 17, 18, 19, 20, 21, 22, 23, 24, 25, 30, 35, 40, 45; in certain embodiments, at least 15; in certain embodiments, at least 16; in certain embodiments, at least 17; in certain embodiments, at least 18; in certain embodiments, at least 19; in certain embodiments, at least 20; in certain embodiments, at least 21; in certain embodiments, at least 22; in certain embodiments, at least 23; in certain embodiments, at least 24; in certain embodiments, at least 25) contiguous bases of a base sequence that is identical to or complementary to a base sequence of a gene or a transcript (e.g., mRNA) thereof.

Base sequences of the guide strand of dsRNAi oligonucleotides, as appreciated by those skilled in the art, typically have sufficient length and complementarity to their targets, e.g., RNA transcripts (e.g., pre-mRNA, mature mRNA, etc.) to mediate target-specific knockdown. In certain embodiments, the base sequence of a dsRNAi oligonucleotide guide strand has a sufficient length and identity to a transcript target to mediate target-specific knockdown. In certain embodiments, the dsRNAi oligonucleotide guide strand is complementary to a portion of a transcript (a transcript target sequence). In certain embodiments, the base sequence of a dsRNAi oligonucleotide has 90% or more identity with the base sequence of a ds oligonucleotide disclosed in Table 1, wherein each T can be independently substituted with U and vice versa. In certain embodiments, the base sequence of a dsRNAi oligonucleotide has 95% or more identity with the base sequence of an oligonucleotide disclosed in Table 1, wherein each T can be independently substituted with U and vice versa. In certain embodiments, the base sequence of a dsRNAi oligonucleotide comprises a continuous span of 15 or more bases of an oligonucleotide disclosed in Table 1, wherein each T can be independently substituted with U and vice versa, except that one or more bases within the span are abasic (e.g., a nucleobase is absent from a nucleotide). In certain embodiments, the base sequence of a dsRNAi oligonucleotide comprises a continuous span of 19 or more bases of a dsRNAi oligonucleotide disclosed herein, except that one or more bases within the span are abasic (e.g., a nucleobase is absent from a nucleotide). In certain embodiments, the base sequence of a dsRNAi oligonucleotide comprises a continuous span of 19 or more bases of a ds oligonucleotide disclosed herein, wherein each T can be independently substituted with U and vice versa, except for a difference in the 1 or 2 bases at the 5′ end and/or 3′ end of the base sequences.

In certain embodiments, the present disclosure pertains to a ds oligonucleotide having a base sequence which comprises the base sequence of any ds oligonucleotide disclosed herein, wherein each T may be independently replaced with U and vice versa.

In certain embodiments, the present disclosure pertains to a ds oligonucleotide having a base sequence which comprises at least 15 contiguous bases of the base sequence of any ds oligonucleotide disclosed herein, wherein each T may be independently replaced with U and vice versa.

In certain embodiments, the present disclosure pertains to a ds oligonucleotide having a base sequence which is at least 90% identical to the base sequence of any ds oligonucleotide disclosed herein, wherein each T may be independently replaced with U and vice versa.

In certain embodiments, the present disclosure pertains to a ds oligonucleotide having a base sequence which is at least 95% identical to the base sequence of any ds oligonucleotide disclosed herein, wherein each T may be independently replaced with U and vice versa.

In certain embodiments, a base sequence of a ds oligonucleotide is, comprises, or comprises 10-20, e.g., 10, 11, 12, 13, 14, 15, 16, 17, 18, 19 or 20 contiguous bases of the base sequence of any ds oligonucleotide described herein, wherein each T may be independently replaced with U and vice versa.

In certain embodiments, a dsRNAi oligonucleotide is selected from Table 1.

In certain embodiments, a dsRNAi oligonucleotide target two or more or all alleles (if multiple alleles exist in a relevant system). In certain embodiments, a ds oligonucleotide reduces expressions, levels and/or activities of both wild-type allele and mutant allele, and/or transcripts and/or products thereof.

In certain embodiments, base sequences of provided ds oligonucleotides are fully complementary to both human and a non-human primate (NHP) target sequences. In certain embodiments, such sequences can be particularly useful as they can be readily assessed in both human and non-human primates.

In certain embodiments, a dsRNAi oligonucleotide comprises a base sequence or portion thereof described in Table 1, wherein each T may be independently replaced with U and vice versa, and/or a sugar, nucleobase, and/or internucleotidic linkage modification and/or a pattern thereof described in Table 1, and/or an additional chemical moiety (in addition to an oligonucleotide chain, e.g., a target moiety, a lipid moiety, a carbohydrate moiety, etc.) described in Table 1.

In certain embodiments, the terms “complementary,” “fully complementary” and “substantially complementary” may be used with respect to the base matching between n ds oligonucleotide (e.g., a dsRNAi oligonucleotide) base sequence and a target sequence, as will be understood by those skilled in the art from the context of their use. It is noted that substitution of T for U, or vice versa, generally does not alter the amount of complementarity. As used herein, a ds oligonucleotide that is “substantially complementary” to a target sequence is largely or mostly complementary but not 100% complementary. In certain embodiments, a sequence (e.g., a dsRNAi oligonucleotide) which is substantially complementary has 1, 2, 3, 4 or 5 mismatches when aligned to its target sequence. In certain embodiments, a dsRNAi oligonucleotide has a base sequence which is substantially complementary to ai target sequence. In certain embodiments, a dsRNAi oligonucleotide has a base sequence which is substantially complementary to the complement of the sequence of a dsRNAi oligonucleotide disclosed herein. As appreciated by those skilled in the art, in certain embodiments, sequences of ds oligonucleotides need not be 100% complementary to their targets for the ds oligonucleotides to perform their functions (e.g., knockdown of target nucleic acids. Typically when determining complementarity, A and T (or U) are complementary nucleobases and C and G are complementary nucleobases.

In certain embodiments, a “portion” (e.g., of a base sequence or a pattern of modifications) is at least 5, 6, 7, 8, 9, 10, 11, 12, 13, 14, 15, 16, 17, 18, 19, or 20 monomeric units long (e.g., for a base sequence, at least 5, 6, 7, 8, 9, 10, 11, 12, 13, 14, 15, 16, 17, 18, 19, or 20 bases long). In certain embodiments, a “portion” of a base sequence is at least 5 bases long. In certain embodiments, a “portion” of a base sequence is at least 10 bases long. In certain embodiments, a “portion” of a base sequence is at least 15 bases long. In certain embodiments, a “portion” of a base sequence is at least 16, 17, 18, 19 or 20 bases long. In certain embodiments, a “portion” of a base sequence is at least 20 bases long. In certain embodiments, a portion of a base sequence is 10, 11, 12, 13, 14, 15, 16, 17, 18, 19 or more contiguous (consecutive) bases. In certain embodiments, a portion of a base sequence is 15 or more contiguous (consecutive) bases. In certain embodiments, a portion of a base sequence is 16, 17, 18, 19 or 20 or more contiguous (consecutive) bases. In certain embodiments, a portion of a base sequence is 20 or more contiguous (consecutive) bases.

In certain embodiments, a portion is a span of at least 15, 16, 17, 18, 19, 20, 21, 22, 23, 24, or 25 total nucleotides. In certain embodiments, a portion is a span of at least 15, 16, 17, 18, 19, 20, 21, 22, 23, 24, or 25 total nucleotides with 0-3 mismatches. In certain embodiments, a portion is a span of at least 15, 16, 17, 18, 19, 20, 21, 22, 23, 24, or 25 total nucleotides with 0-3 mismatches, wherein a span with 0 mismatches is complementary and a span with 1 or more mismatches is a non-limiting example of substantial complementarity. In certain embodiments, a base comprises a portion characteristic of a nucleic acid (e.g., a gene) in that the portion is identical or complementary to a portion of the nucleic acid or a transcript thereof, and is not identical or complementary to a portion of any other nucleic acid (e.g., a gene) or a transcript thereof in the same genome. In certain embodiments, a portion is characteristic of human dsRNAi.

In certain embodiments, a provided oligonucleotide, e.g., a dsRNAi oligonucleotide, has a length of no more than about 49, 45, 40, 30, 35, 25, or 23 total nucleotides as described herein. In certain embodiments, wherein the sequence recited herein starts with a U or T at the 5′-end, the U can be deleted and/or replaced by another base.

In certain embodiments, ds oligonucleotides, e.g., dsRNAi oligonucleotides are stereorandom. In certain embodiments, RNAi oligonucleotides are chirally controlled. In certain embodiments, a ds RNAi oligonucleotide is chirally pure (or “stereopure”, “stereochemically pure”), wherein the ds oligonucleotide exists as a single stereoisomeric form (in many cases a single diastereoisomeric (or “diastereomeric”) form as multiple chiral centers may exist in a ds oligonucleotide, e.g., at linkage phosphorus, sugar carbon, etc.). As appreciated by those skilled in the art, a chirally pure ds oligonucleotide is separated from its other stereoisomeric forms (to the extent that some impurities may exist as chemical and biological processes, selectivities and/or purifications etc. rarely, if ever, go to absolute completeness). In a chirally pure ds oligonucleotide, each chiral center is independently defined with respect to its configuration (for a chirally pure ds oligonucleotide, each internucleotidic linkage is independently stereodefined or chirally controlled). In contrast to chirally controlled and chirally pure ds oligonucleotides which comprise stereodefined linkage phosphorus, racemic (or “stereorandom”, “non-chirally controlled”) ds oligonucleotides comprising chiral linkage phosphorus, e.g., from traditional phosphoramidite oligonucleotide synthesis without stereochemical control during coupling steps in combination with traditional sulfurization (creating stereorandom phosphorothioate internucleotidic linkages), refer to a random mixture of various stereoisomers (typically diastereoisomers (or “diastereomers”) as there are multiple chiral centers in a ds oligonucleotide; e.g., from traditional ds oligonucleotide preparation using reagents containing no chiral elements other than those in nucleosides and linkage phosphorus). For example, for A*A*A wherein * is a phosphorothioate internucleotidic linkage (which comprises a chiral linkage phosphorus), a racemic oligonucleotide preparation includes four diastereomers [2²=4, considering the two chiral linkage phosphorus, each of which can exist in either of two configurations (Sp or Rp)]: A*SA*SA, A*SA*RA, A*RA*SA, and A*RA*RA, wherein *S represents a Sp phosphorothioate internucleotidic linkage and *R represents a Rp phosphorothioate internucleotidic linkage. For a chirally pure oligonucleotide, e.g., A*SA*SA, it exists in a single stereoisomeric form and it is separated from the other stereoisomers (e.g., the diastereomers A*SA*RA, A*RA*SA, and A*RA*RA).

In certain embodiments, dsRNAi oligonucleotides comprise 1, 2, 3, 4, 5, 6, 7, 8, 9, 10 or more stereorandom internucleotidic linkages (mixture of Rp and Sp linkage phosphorus at the internucleotidic linkage, e.g., from traditional non-chirally controlled oligonucleotide synthesis). In certain embodiments, dsRNAi oligonucleotides comprise one or more (e.g., 1-50, 1-40, 1-30, 1-25, 1-20, 1, 2, 3, 4, 5, 6, 7, 8, 9, 10, 11, 12, 13, 14, 15, 16, 17, 18, 19, 20, 21, 22, 23, 24, 25 or more) chirally controlled internucleotidic linkages (Rp or Sp linkage phosphorus at the internucleotidic linkage, e.g., from chirally controlled oligonucleotide synthesis).

In certain embodiments, an internucleotidic linkage is a phosphorothioate internucleotidic linkage. In certain embodiments, an internucleotidic linkage is a stereorandom phosphorothioate internucleotidic linkage. In certain embodiments, an internucleotidic linkage is a chirally controlled phosphorothioate internucleotidic linkage.

Among other things, the present disclosure provides technologies for preparing chirally controlled (in certain embodiments, stereochemically pure) ds oligonucleotides. In certain embodiments, ds oligonucleotides are stereochemically pure. In certain embodiments, ds oligonucleotides of the present disclosure are about 5%-100%, 10%-100%, 20%-100%, 30%-100%, 40%-100%, 50%-100%, 60%-100%, 70%-100%, 80-100%, 90-100%, 95-100%, 50%-90%, or about 5%, 10%, 15%, 20%, 25%, 30%, 35%, 40%, 45%, 50%, 55%, 60%, 65%, 70%, 75%, 80%, 85%, 90%, 95%, or 100%, or at least about 5%, 10%, 15%, 20%, 25%, 30%, 35%, 40%, 45%, 50%, 55%, 60%, 65%, 70%, 75%, 80%, 85%, 90%, 95%, or 99%, pure. In certain embodiments, internucleotidic linkages of ds oligonucleotides comprise or consist of one or more (e.g., 1-50, 1-40, 1-30, 1-25, 1-20, 5-50, 5-40, 5-30, 5-25, 5-20, 1, 2, 3, 4, 5, 6, 7, 8, 9, 10, 11, 12, 13, 14, 15, 16, 17, 18, 19, 20, 21, 22, 23, 24, 25 or more) chiral internucleotidic linkages, each of which independently has a diastereopurity of at least 80%, 85%, 90%, 91%, 92%, 93%, 94%, 95%, 96%, 97%, 98%, 99% or 99.5%, typically at least 90%, 91%, 92%, 93%, 94%, 95%, 96%, 97%, 98%, 99% or 99.5%. In certain embodiments, ds oligonucleotides of the present disclosure, e.g., dsRNAi oligonucleotides, have a diastereopurity of (DS)^{CIL}, wherein DS is a diastereopurity as described in the present disclosure (e.g., 90%, 91%, 92%, 93%, 94%, 95%, 96%, 97%, 98%, 99% or 99.5% or more) and CIL is the number of chirally controlled internucleotidic linkages (e.g., 1-50, 1-40, 1-30, 1-25, 1-20, 5-50, 5-40, 5-30, 5-25, 5-20, 1, 2, 3, 4, 5, 6, 7, 8, 9, 10, 11, 12, 13, 14, 15, 16, 17, 18, 19, 20, 21, 22, 23, 24, 25 or more). In certain embodiments, DS is 95%-100%. In certain embodiments, each internucleotidic linkage is independently chirally controlled, and CIL is the number of chirally controlled internucleotidic linkages.

As examples, certain dsRNAi oligonucleotides comprising certain example base sequences, nucleobase modifications and patterns thereof, sugar modifications and patterns thereof, internucleotidic linkages and patterns thereof, linkage phosphorus stereochemistry and patterns thereof, linkers, and/or additional chemical moieties are presented in Table 1, below. Among other things, ds oligonucleotides, e.g., those in Table 1A, may be utilized to target a transcript, e.g., to reduce the level of a transcript and/or a product thereof

TABLE 1
Example Oligonucleotides/Compositions that target TTR.

| | | | Stereochemistry/ |
|---|---|---|---|
| ID | Description | Naked Sequence | linkage |
| WV-46497 | mUn001RfU*SmAmUmAfGmAmGmCmAmAmGmAfAmCfAmCmUmG | UUAUAGAGCAAGAACACUGUU | nRSOOOOOOOOOOOOOOOO |
| | mUmU*SmU*SmU | UU | OOSS |
| WV-46498 | mU*RfUn001RmAmUmAfGmAmGmCmAmAmGmAfAmCfAmCmUmG | UUAUAGAGCAAGAACACUGUU | RnROOOOOOOOOOOOOOOO |
| | mUmU*SmU*SmU | UU | OOSS |
| WV-46499 | mU*RfU*SmAn001RmUmAfGmAmGmCmAmAmGmAfAmCfAmCmU | UUAUAGAGCAAGAACACUGUU | RSnROOOOOOOOOOOOOOO |
| | mGmUmU*SmU*SmU | UU | OOSS |
| WV-46500 | mU*RfU*SmAmUn001RmAfGmAmGmCmAmAmGmAfAmCfAmCmU | UUAUAGAGCAAGAACACUGUU | RSOnROOOOOOOOOOOOOO |
| | mGmUmU*SmU*SmU | UU | OOSS |
| WV-46501 | mU*RfU*SmAmUmAn001RfGmAmGmCmAmAmGmAfAmCfAmCmU | UUAUAGAGCAAGAACACUGUU | RSOOnROOOOOOOOOOOOO |
| | mGmUmU*SmU*SmU | UU | OOSS |
| WV-46502 | mU*RfU*SmAmUmAfGn001RmAmGmCmAmAmGmAfAmCfAmCmU | UUAUAGAGCAAGAACACUGUU | RSOOOnROOOOOOOOOOOO |
| | mGmUmU*SmU*SmU | UU | OOSS |
| WV-46503 | mU*RfU*SmAmUmAfGmAn001RmGmCmAmAmGmAfAmCfAmCmU | UUAUAGAGCAAGAACACUGUU | RSOOOOnROOOOOOOOOOO |
| | mGmUmU*SmU*SmU | UU | OOSS |
| WV-46504 | mU*RfU*SmAmUmAfGmAmGn001RmCmAmAmGmAfAmCfAmCmU | UUAUAGAGCAAGAACACUGUU | RSOOOOOnROOOOOOOOOO |
| | mGmUmU*SmU*SmU | UU | OOSS |
| WV-46505 | mU*RfU*SmAmUmAfGmAmGmCn001RmAmAmGmAfAmCfAmCmU | UUAUAGAGCAAGAACACUGUU | RSOOOOOOnROOOOOOOOO |
| | mGmUmU*SmU*SmU | UU | OOSS |
| WV-46506 | mU*RfU*SmAmUmAfGmAmGmCmAn001RmAmGmAfAmCfAmCmU | UUAUAGAGCAAGAACACUGUU | RSOOOOOOOnROOOOOOOO |
| | mGmUmU*SmU*SmU | UU | OOSS |
| WV-46507 | mU*RfU*SmAmUmAfGmAmGmCmAmAn001RmGmAfAmCfAmCmU | UUAUAGAGCAAGAACACUGUU | RSOOOOOOOOnROOOOOOO |
| | mGmUmU*SmU*SmU | UU | OOSS |
| WV-46508 | mU*RfU*SmAmUmAfGmAmGmCmAmAmGn001RmAfAmCfAmCmU | UUAUAGAGCAAGAACACUGUU | RSOOOOOOOOOnROOOOOO |
| | mGmUmU*SmU*SmU | UU | OOSS |
| WV-46509 | mU*RfU*SmAmUmAfGmAmGmCmAmAmGmAn001RfAmCfAmCmU | UUAUAGAGCAAGAACACUGUU | RSOOOOOOOOOOnROOOOO |
| | mGmUmU*SmU*SmU | UU | OOSS |
| WV-46510 | mU*RfU*SmAmUmAfGmAmGmCmAmAmGmAfAn001RmCfAmCmU | UUAUAGAGCAAGAACACUGUU | RSOOOOOOOOOOOnROOOO |
| | mGmUmU*SmU*SmU | UU | OOSS |
| WV-46511 | mU*RfU*SmAmUmAfGmAmGmCmAmAmGmAfAmCn001RfAmCmU | UUAUAGAGCAAGAACACUGUU | RSOOOOOOOOOOOOnROOO |
| | mGmUmU*SmU*SmU | UU | OOSS |
| WV-46512 | mU*RfU*SmAmUmAfGmAmGmCmAmAmGmAfAmCfAn001RmCmU | UUAUAGAGCAAGAACACUGUU | RSOOOOOOOOOOOOOnROO |
| | mGmUmU*SmU*SmU | UU | OOSS |
| WV-46513 | mU*RfU*SmAmUmAfGmAmGmCmAmAmGmAfAmCfAmCn001RmU | UUAUAGAGCAAGAACACUGUU | RSOOOOOOOOOOOOOOnRO |
| | mGmUmU*SmU*SmU | UU | OOSS |
| WV-46514 | mU*RfU*SmAmUmAfGmAmGmCmAmAmGmAfAmCfAmCmUn001R | UUAUAGAGCAAGAACACUGUU | RSOOOOOOOOOOOOOOOnR |
| | mGmUmU*SmU*SmU | UU | OOSS |
| WV-46515 | mU*RfU*SmAmUmAfGmAmGmCmAmAmGmAfAmCfAmCmUmGn00 | UUAUAGAGCAAGAACACUGUU | RSOOOOOOOOOOOOOOOOn |
| | 1RmUmU*SmU*SmU | UU | ROSS |
| WV-46516 | mU*RfU*SmAmUmAfGmAmGmCmAmAmGmAfAmCfAmCmUmGmUn | UUAUAGAGCAAGAACACUGUU | RSOOOOOOOOOOOOOOOOO |
| | 001RmU*SmU*SmU | UU | nRSS |
| WV-46517 | mU*RfU*SmAmUmAfGmAmGmCmAmAmGmAfAmCfAmCmUmGmUm | UUAUAGAGCAAGAACACUGUU | RSOOOOOOOOOOOOOOOOO |
| | Un001RmU*SmU | UU | OnRS |
| WV-46518 | mU*RfU*SmAmUmAfGmAmGmCmAmAmGmAfAmCfAmCmUmGmUm | UUAUAGAGCAAGAACACUGUU | RSOOOOOOOOOOOOOOOOO |
| | U*SmUn001RmU | UU | OSnR |
| WV-46519 | mUn001SfU*SmAmUmAfGmAmGmCmAmAmGmAfAmCfAmCmUmG | UUAUAGAGCAAGAACACUGUU | nSSOOOOOOOOOOOOOOOO |
| | mUmU*SmU*SmU | UU | OOSS |
| WV-46520 | mU*RfUn001SmAmUmAfGmAmGmCmAmAmGmAfAmCfAmCmUmG | UUAUAGAGCAAGAACACUGUU | RnSOOOOOOOOOOOOOOOO |
| | mUmU*SmU*SmU | UU | OOSS |
| WV-45148 | mU*RfU*SmAn001SmUmAfGmAmGmCmAmAmGmAfAmCfAmCmU | UUAUAGAGCAAGAACACUGUU | RSnSOOOOOOOOOOOOOOO |
| | mGmUmU*SmU*SmU | UU | OOSS |
| WV-46521 | mU*RfU*SmAmUn001SmAfGmAmGmCmAmAmGmAfAmCfAmCmU | UUAUAGAGCAAGAACACUGUU | RSOnSOOOOOOOOOOOOOO |
| | mGmUmU*SmU*SmU | UU | OOSS |
| WV-46522 | mU*RfU*SmAmUmAn001SfGmAmGmCmAmAmGmAfAmCfAmCmU | UUAUAGAGCAAGAACACUGUU | RSOOnSOOOOOOOOOOOOO |
| | mGmUmU*SmU*SmU | UU | OOSS |
| WV-46523 | mU*RfU*SmAmUmAfGn001SmAmGmCmAmAmGmAfAmCfAmCmU | UUAUAGAGCAAGAACACUGUU | RSOOOnSOOOOOOOOOOOO |
| | mGmUmU*SmU*SmU | UU | OOSS |
| WV-46524 | mU*RfU*SmAmUmAfGmAn001SmGmCmAmAmGmAfAmCfAmCmU | UUAUAGAGCAAGAACACUGUU | RSOOOOnSOOOOOOOOOOO |
| | mGmUmU*SmU*SmU | UU | OOSS |
| WV-46525 | mU*RfU*SmAmUmAfGmAmGn001SmCmAmAmGmAfAmCfAmCmU | UUAUAGAGCAAGAACACUGUU | RSOOOOOnSOOOOOOOOOO |
| | mGmUmU*SmU*SmU | UU | OOSS |
| WV-46526 | mU*RfU*SmAmUmAfGmAmGmCn001SmAmAmGmAfAmCfAmCmU | UUAUAGAGCAAGAACACUGUU | RSOOOOOOnSOOOOOOOOO |
| | mGmUmU*SmU*SmU | UU | OOSS |
| WV-45147 | mU*RfU*SmAmUmAfGmAmGmCmAn001SmAmGmAfAmCfAmCmU | UUAUAGAGCAAGAACACUGUU | RSOOOOOOOnSOOOOOOOO |
| | mGmUmU*SmU*SmU | UU | OOSS |
| WV-46527 | mU*RfU*SmAmUmAfGmAmGmCmAmAn001SmGmAfAmCfAmCmU | UUAUAGAGCAAGAACACUGUU | RSOOOOOOOOnSOOOOOOO |
| | mGmUmU*SmU*SmU | UU | OOSS |
| WV-46528 | mU*RfU*SmAmUmAfGmAmGmCmAmAmGn001SmAfAmCfAmCmU | UUAUAGAGCAAGAACACUGUU | RSOOOOOOOOOn$OOOOOO |
| | mGmUmU*SmU*SmU | UU | OOSS |
| WV-46529 | mU*RfU*SmAmUmAfGmAmGmCmAmAmGmAn001SfAmCfAmCmU | UUAUAGAGCAAGAACACUGUU | RSOOOOOOOOOOnSOOOOO |
| | mGmUmU*SmU*SmU | UU | OOSS |
| WV-46530 | mU*RfU*SmAmUmAfGmAmGmCmAmAmGmAfAn001SmCfAmCmU | UUAUAGAGCAAGAACACUGUU | RSOOOOOOOOOOOnSOOOO |
| | mGmUmU*SmU*SmU | UU | OOSS |
| WV-46531 | mU*RfU*SmAmUmAfGmAmGmCmAmAmGmAfAmCn001SfAmCmU | UUAUAGAGCAAGAACACUGUU | RSOOOOOOOOOOOOnSOOO |
| | mGmUmU*SmU*SmU | UU | OOSS |
| WV-46532 | mU*RfU*SmAmUmAfGmAmGmCmAmAmGmAfAmCfAn001SmCmU | UUAUAGAGCAAGAACACUGUU | RSOOOOOOOOOOOOOnSOO |
| | mGmUmU*SmU*SmU | UU | OOSS |
| WV-46533 | mU*RfU*SmAmUmAfGmAmGmCmAmAmGmAfAmCfAmCn001SmU | UUAUAGAGCAAGAACACUGUU | RSOOOOOOOOOOOOOOnSO |
| | mGmUmU*SmU*SmU | UU | OOSS |
| WV-46534 | mU*RfU*SmAmUmAfGmAmGmCmAmAmGmAfAmCfAmCmUn001S | UUAUAGAGCAAGAACACUGUU | RSOOOOOOOOOOOOOOOnS |
| | mGmUmU*SmU*SmU | UU | OOSS |
| WV-45146 | mU*RfU*SmAmUmAfGmAmGmCmAmAmGmAfAmCfAmCmUmGn00 | UUAUAGAGCAAGAACACUGUU | RSOOOOOOOOOOOOOOOOn |
| | 1SmUmU*SmU*SmU | UU | SOSS |
| WV-46535 | mU*RfU*SmAmUmAfGmAmGmCmAmAmGmAfAmCfAmCmUmGmUn | UUAUAGAGCAAGAACACUGUU | RSOOOOOOOOOOOOOOOOO |
| | 001SmU*SmU*SmU | UU | nSSS |
| WV-46536 | mU*RfU*SmAmUmAfGmAmGmCmAmAmGmAfAmCfAmCmUmGmUm | UUAUAGAGCAAGAACACUGUU | RSOOOOOOOOOOOOOOOOO |
| | Un001SmU*SmU | UU | OnSS |
| WV-46537 | mU*RfU*SmAmUmAfGmAmGmCmAmAmGmAfAmCfAmCmUmGmUm | UUAUAGAGCAAGAACACUGUU | RSOOOOOOOOOOOOOOOOO |
| | U*SmUn001SmU | UU | OSnS |
| WV-46538 | mUn001RfU*RmAmUmAfGmAmGmCmAmAmGmAfAmCfAmCmUmG | UUAUAGAGCAAGAACACUGUU | nRROOOOOOOOOOOOOOOO |
| | mUmU*SmU*SmU | UU | OOSS |
| WV-46539 | mU*SfUn001RmAmUmAfGmAmGmCmAmAmGmAfAmCfAmCmUmG | UUAUAGAGCAAGAACACUGUU | SnROOOOOOOOOOOOOOOO |
| | mUmU*SmU*SmU | UU | OOSS |
| WV-46540 | mU*SfU*RmAn001RmUmAfGmAmGmCmAmAmGmAfAmCfAmCmU | UUAUAGAGCAAGAACACUGUU | SRnROOOOOOOOOOOOOOO |
| | mGmUmU*SmU*SmU | UU | OOSS |
| WV-46541 | mU*SfU*RmAmUn001RmAfGmAmGmCmAmAmGmAfAmCfAmCmU | UUAUAGAGCAAGAACACUGUU | SROnROOOOOOOOOOOOOO |
| | mGmUmU*SmU*SmU | UU | OOSS |
| WV-46542 | mU*SfU*RmAmUmAn001RfGmAmGmCmAmAmGmAfAmCfAmCmU | UUAUAGAGCAAGAACACUGUU | SROOnROOOOOOOOOOOOO |
| | mGmUmU*SmU*SmU | UU | OOSS |
| WV-46543 | mU*SfU*RmAmUmAfGn001RmAmGmCmAmAmGmAfAmCfAmCmU | UUAUAGAGCAAGAACACUGUU | SROOOnROOOOOOOOOOOO |
| | mGmUmU*SmU*SmU | UU | OOSS |
| WV-46544 | mU*SfU*RmAmUmAfGmAn001RmGmCmAmAmGmAfAmCfAmCmU | UUAUAGAGCAAGAACACUGUU | SROOOOnROOOOOOOOOOO |
| | mGmUmU*SmU*SmU | UU | OOSS |
| WV-46545 | mU*SfU*RmAmUmAfGmAmGn001RmCmAmAmGmAfAmCfAmCmU | UUAUAGAGCAAGAACACUGUU | SROOOOOnROOOOOOOOOO |
| | mGmUmU*SmU*SmU | UU | OOSS |
| WV-46546 | mU*SfU*RmAmUmAfGmAmGmCn001RmAmAmGmAfAmCfAmCmU | UUAUAGAGCAAGAACACUGUU | SROOOOOOnROOOOOOOOO |
| | mGmUmU*SmU*SmU | UU | OOSS |
| WV-46547 | mU*SfU*RmAmUmAfGmAmGmCmAn001RmAmGmAfAmCfAmCmU | UUAUAGAGCAAGAACACUGUU | SROOOOOOOnROOOOOOOO |
| | mGmUmU*SmU*SmU | UU | OOSS |
| WV-46548 | mU*SfU*RmAmUmAfGmAmGmCmAmAn001RmGmAfAmCfAmCmU | UUAUAGAGCAAGAACACUGUU | SROOOOOOOOnROOOOOOO |
| | mGmUmU*SmU*SmU | UU | OOSS |
| WV-46549 | mU*SfU*RmAmUmAfGmAmGmCmAmAmGn001RmAfAmCfAmCmU | UUAUAGAGCAAGAACACUGUU | SROOOOOOOOOnROOOOOO |
| | mGmUmU*SmU*SmU | UU | OOSS |
| WV-46550 | mU*SfU*RmAmUmAfGmAmGmCmAmAmGmAn001RfAmCfAmCmU | UUAUAGAGCAAGAACACUGUU | SROOOOOOOOOOnROOOOO |
| | mGmUmU*SmU*SmU | UU | OOSS |
| WV-46551 | mU*SfU*RmAmUmAfGmAmGmCmAmAmGmAfAn001RmCfAmCmU | UUAUAGAGCAAGAACACUGUU | SROOOOOOOOOOOnROOOO |
| | mGmUmU*SmU*SmU | UU | OOSS |
| WV-46552 | mU*SfU*RmAmUmAfGmAmGmCmAmAmGmAfAmCn001RfAmCmU | UUAUAGAGCAAGAACACUGUU | SROOOOOOOOOOOOnROOO |
| | mGmUmU*SmU*SmU | UU | OOSS |
| WV-46553 | mU*SfU*RmAmUmAfGmAmGmCmAmAmGmAfAmCfAn001RmCmU | UUAUAGAGCAAGAACACUGUU | SROOOOOOOOOOOOOnROO |
| | mGmUmU*SmU*SmU | UU | OOSS |
| WV-46554 | mU*SfU*RmAmUmAfGmAmGmCmAmAmGmAfAmCfAmCn001RmU | UUAUAGAGCAAGAACACUGUU | SROOOOOOOOOOOOOOnRO |
| | mGmUmU*SmU*SmU | UU | OOSS |
| WV-46555 | mU*SfU*RmAmUmAfGmAmGmCmAmAmGmAfAmCfAmCmUn001R | UUAUAGAGCAAGAACACUGUU | SROOOOOOOOOOOOOOOnR |
| | mGmUmU*SmU*SmU | UU | OOSS |
| WV-46556 | mU*SfU*RmAmUmAfGmAmGmCmAmAmGmAfAmCfAmCmUmGn00 | UUAUAGAGCAAGAACACUGUU | SROOOOOOOOOOOOOOOOn |
| | 1RmUmU*SmU*SmU | UU | ROSS |
| WV-46557 | mU*SfU*RmAmUmAfGmAmGmCmAmAmGmAfAmCfAmCmUmGmUn | UUAUAGAGCAAGAACACUGUU | SROOOOOOOOOOOOOOOOO |
| | 001RmU*SmU*SmU | UU | nRSS |
| WV-46558 | mU*SfU*RmAmUmAfGmAmGmCmAmAmGmAfAmCfAmCmUmGmUm | UUAUAGAGCAAGAACACUGUU | SROOOOOOOOOOOOOOOOO |
| | Un001RmU*SmU | UU | OnRS |
| WV-46559 | mU*SfU*RmAmUmAfGmAmGmCmAmAmGmAfAmCfAmCmUmGmUm | UUAUAGAGCAAGAACACUGUU | SROOOOOOOOOOOOOOOOO |
| | U*SmUn001RmU | UU | OSnR |
| WV-46560 | mUn001SfU*RmAmUmAfGmAmGmCmAmAmGmAfAmCfAmCmUmG | UUAUAGAGCAAGAACACUGUU | nSROOOOOOOOOOOOOOOO |
| | mUmU*SmU*SmU | UU | OOSS |
| WV-46561 | mU*SfUn001SmAmUmAfGmAmGmCmAmAmGmAfAmCfAmCmUmG | UUAUAGAGCAAGAACACUGUU | SnSOOOOOOOOOOOOOOOO |
| | mUmU*SmU*SmU | UU | OOSS |
| WV-44453 | mU*SfU*RmAn001SmUmAfGmAmGmCmAmAmGmAfAmCfAmCmU | UUAUAGAGCAAGAACACUGUU | SRnSOOOOOOOOOOOOOOO |
| | mGmUmU*SmU*SmU | UU | OOSS |
| WV-46562 | mU*SfU*RmAmUn001SmAfGmAmGmCmAmAmGmAfAmCfAmCmU | UUAUAGAGCAAGAACACUGUU | SROnSOOOOOOOOOOOOOO |
| | mGmUmU*SmU*SmU | UU | OOSS |
| WV-46563 | mU*SfU*RmAmUmAn001SfGmAmGmCmAmAmGmAfAmCfAmCmU | UUAUAGAGCAAGAACACUGUU | SROOnSOOOOOOOOOOOOO |
| | mGmUmU*SmU*SmU | UU | OOSS |
| WV-46564 | mU*SfU*RmAmUmAfGn001SmAmGmCmAmAmGmAfAmCfAmCmU | UUAUAGAGCAAGAACACUGUU | SROOOnSOOOOOOOOOOOO |
| | mGmUmU*SmU*SmU | UU | OOSS |
| WV-46565 | mU*SfU*RmAmUmAfGmAn001SmGmCmAmAmGmAfAmCfAmCmU | UUAUAGAGCAAGAACACUGUU | SROOOOnSOOOOOOOOOOO |
| | mGmUmU*SmU*SmU | UU | OOSS |
| WV-46566 | mU*SfU*RmAmUmAfGmAmGn001SmCmAmAmGmAfAmCfAmCmU | UUAUAGAGCAAGAACACUGUU | SROOOOOnSOOOOOOOOOO |
| | mGmUmU*SmU*SmU | UU | OOSS |
| WV-46567 | mU*SfU*RmAmUmAfGmAmGmCn001SmAmAmGmAfAmCfAmCmU | UUAUAGAGCAAGAACACUGUU | SROOOOOOnSOOOOOOOOO |
| | mGmUmU*SmU*SmU | UU | OOSS |
| WV-44452 | mU*SfU*RmAmUmAfGmAmGmCmAn001SmAmGmAfAmCfAmCmU | UUAUAGAGCAAGAACACUGUU | SROOOOOOOnSOOOOOOOO |
| | mGmUmU*SmU*SmU | UU | OOSS |
| WV-46568 | mU*SfU*RmAmUmAfGmAmGmCmAmAn001SmGmAfAmCfAmCmU | UUAUAGAGCAAGAACACUGUU | SROOOOOOOOnSOOOOOOO |
| | mGmUmU*SmU*SmU | UU | OOSS |
| WV-46569 | mU*SfU*RmAmUmAfGmAmGmCmAmAmGn001SmAfAmCfAmCmU | UUAUAGAGCAAGAACACUGUU | SROOOOOOOOOnSOOOOOO |
| | mGmUmU*SmU*SmU | UU | OOSS |
| WV-46570 | mU*SfU*RmAmUmAfGmAmGmCmAmAmGmAn001SfAmCfAmCmU | UUAUAGAGCAAGAACACUGUU | SROOOOOOOOOOnSOOOOO |
| | mGmUmU*SmU*SmU | UU | OOSS |
| WV-46571 | mU*SfU*RmAmUmAfGmAmGmCmAmAmGmAfAn001SmCfAmCmU | UUAUAGAGCAAGAACACUGUU | SROOOOOOOOOOOn$OOOO |
| | mGmUmU*SmU*SmU | UU | OOSS |
| WV-46572 | mU*SfU*RmAmUmAfGmAmGmCmAmAmGmAfAmCn001SfAmCmU | UUAUAGAGCAAGAACACUGUU | SROOOOOOOOOOOOnSOOO |
| | mGmUmU*SmU*SmU | UU | OOSS |
| WV-46573 | mU*SfU*RmAmUmAfGmAmGmCmAmAmGmAfAmCfAn001SmCmU | UUAUAGAGCAAGAACACUGUU | SROOOOOOOOOOOOOnSOO |
| | mGmUmU*SmU*SmU | UU | OOSS |
| WV-46574 | mU*SfU*RmAmUmAfGmAmGmCmAmAmGmAfAmCfAmCn001SmU | UUAUAGAGCAAGAACACUGUU | SROOOOOOOOOOOOOOnSO |
| | mGmUmU*SmU*SmU | UU | OOSS |
| WV-46575 | mU*SfU*RmAmUmAfGmAmGmCmAmAmGmAfAmCfAmCmUn001S | UUAUAGAGCAAGAACACUGUU | SROOOOOOOOOOOOOOOnS |
| | mGmUmU*SmU*SmU | UU | OOSS |
| WV-44451 | mU*SfU*RmAmUmAfGmAmGmCmAmAmGmAfAmCfAmCmUmGn00 | UUAUAGAGCAAGAACACUGUU | SROOOOOOOOOOOOOOOOn |
| | 1SmUmU*SmU*SmU | UU | SOSS |
| WV-46576 | mU*SfU*RmAmUmAfGmAmGmCmAmAmGmAfAmCfAmCmUmGmUn | UUAUAGAGCAAGAACACUGUU | SROOOOOOOOOOOOOOOOO |
| | 001SmU*SmU*SmU | UU | nSSS |
| WV-46577 | mU*SfU*RmAmUmAfGmAmGmCmAmAmGmAfAmCfAmCmUmGmUm | UUAUAGAGCAAGAACACUGUU | SROOOOOOOOOOOOOOOOO |
| | Un001SmU*SmU | UU | OnSS |
| WV-44457 | mU*SfU*RmAmUmAfGmAmGmCmAmAmGmAfAmCfAmCmUmGmUm | UUAUAGAGCAAGAACACUGUU | SROOOOOOOOOOOOOOOOO |
| | U*SmUn001SmU | UU | OSnS |
| WV-43774 | mU*RfU*SmAmUmAfGmAmGmCmAmAmGmAfAmCfAmCmUmGmUm | UUAUAGAGCAAGAACACUGUU | RSOOOOOOOOOOOOOOOOO |
| | U*SmU*SmU | UU | OSS |
| WV-47066 | mU*RfUn001RfAmUmAfGmAmGmCmAmAmGmAfAmCfAmCmUmG | UUAUAGAGCAAGAACACUGUU | RnROOOOOOOOOOOOOOOO |
| | mUmU*SmU*SmU | UU | OOSS |
| WV-47067 | mU*RfU*SmAn001RfUmAfGmAmGmCmAmAmGmAfAmCfAmCmU | UUAUAGAGCAAGAACACUGUU | RSnROOOOOOOOOOOOOOO |
| | mGmUmU*SmU*SmU | UU | OOSS |
| WV-47068 | mU*RfU*SmAmUn001RfAfGmAmGmCmAmAmGmAfAmCfAmCmU | UUAUAGAGCAAGAACACUGUU | RSOnROOOOOOOOOOOOOO |
| | mGmUmU*SmU*SmU | UU | OOSS |
| WV-47069 | mU*RfU*SmAmUmAfGn001RfAmGmCmAmAmGmAfAmCfAmCmU | UUAUAGAGCAAGAACACUGUU | RSOOOnROOOOOOOOOOOO |
| | mGmUmU*SmU*SmU | UU | OOSS |
| WV-47070 | mU*RfU*SmAmUmAfGmAn001RfGmCmAmAmGmAfAmCfAmCmU | UUAUAGAGCAAGAACACUGUU | RSOOOOnROOOOOOOOOOO |
| | mGmUmU*SmU*SmU | UU | OOSS |
| WV-47071 | mU*RfU*SmAmUmAfGmAmGn001RfCmAmAmGmAfAmCfAmCmU | UUAUAGAGCAAGAACACUGUU | RSOOOOOnROOOOOOOOOO |
| | mGmUmU*SmU*SmU | UU | OOSS |
| WV-47072 | mU*RfU*SmAmUmAfGmAmGmCn001RfAmAmGmAfAmCfAmCmU | UUAUAGAGCAAGAACACUGUU | RSOOOOOOnROOOOOOOOO |
| | mGmUmU*SmU*SmU | UU | OOSS |
| WV-47073 | mU*RfU*SmAmUmAfGmAmGmCmAn001RfAmGmAfAmCfAmCmU | UUAUAGAGCAAGAACACUGUU | RSOOOOOOOnROOOOOOOO |
| | mGmUmU*SmU*SmU | UU | OOSS |
| WV-47074 | mU*RfU*SmAmUmAfGmAmGmCmAmAn001RfGmAfAmCfAmCmU | UUAUAGAGCAAGAACACUGUU | RSOOOOOOOOnROOOOOOO |
| | mGmUmU*SmU*SmU | UU | OOSS |
| WV-47075 | mU*RfU*SmAmUmAfGmAmGmCmAmAmGn001RfAfAmCfAmCmU | UUAUAGAGCAAGAACACUGUU | RSOOOOOOOOOnROOOOOO |
| | mGmUmU*SmU*SmU | UU | OOSS |
| WV-47076 | mU*RfU*SmAmUmAfGmAmGmCmAmAmGmAfAn001RfCfAmCmU | UUAUAGAGCAAGAACACUGUU | RSOOOOOOOOOOOnROOOO |
| | mGmUmU*SmU*SmU | UU | oOSS |
| WV-47077 | mU*RfU*SmAmUmAfGmAmGmCmAmAmGmAfAmCfAn001RfCmU | UUAUAGAGCAAGAACACUGUU | RSOOOOOOOOOOOOOnROO |
| | mGmUmU*SmU*SmU | UU | OOSS |
| WV-47078 | mU*RfU*SmAmUmAfGmAmGmCmAmAmGmAfAmCfAmCn001RfU | UUAUAGAGCAAGAACACUGUU | RSOOOOOOOOOOOOOOnRO |
| | mGmUmU*SmU*SmU | UU | OOSS |
| WV-47079 | mU*RfU*SmAmUmAfGmAmGmCmAmAmGmAfAmCfAmCmUn001R | UUAUAGAGCAAGAACACUGUU | RSOOOOOOOOOOOOOOOnR |
| | fGmUmU*SmU*SmU | UU | OOSS |
| WV-47080 | mU*RfU*SmAmUmAfGmAmGmCmAmAmGmAfAmCfAmCmUmGn00 | UUAUAGAGCAAGAACACUGUU | RSOOOOOOOOOOOOOOOOn |
| | 1RfUmU*SmU*SmU | UU | ROSS |
| WV-47081 | mU*RfU*SmAmUmAfGmAmGmCmAmAmGmAfAmCfAmCmUmGmUn | UUAUAGAGCAAGAACACUGUU | RSOOOOOOOOOOOOOOOOO |
| | 001RfU*SmU*SmU | UU | nRSS |
| WV-47082 | mU*RfU*SmAmUmAfGmAmGmCmAmAmGmAfAmCfAmCmUmGmUm | UUAUAGAGCAAGAACACUGUU | RSOOOOOOOOOOOOOOOOO |
| | Un001RfU*SmU | UU | OnRS |
| WV-47083 | mU*RfU*SmAmUmAfGmAmGmCmAmAmGmAfAmCfAmCmUmGmUm | UUAUAGAGCAAGAACACUGUU | RSOOOOOOOOOOOOOOOOO |
| | U*SmUn001RfU | UU | OSnR |
| WV-47084 | mU*RfUn001SfAmUmAfGmAmGmCmAmAmGmAfAmCfAmCmUmG | UUAUAGAGCAAGAACACUGUU | RnSOOOOOOOOOOOOOOOO |
| | mUmU*SmU*SmU | UU | OOSS |
| WV-47085 | mU*RfU*SmAn001SfUmAfGmAmGmCmAmAmGmAfAmCfAmCmU | UUAUAGAGCAAGAACACUGUU | RSnSOOOOOOOOOOOOOOO |
| | mGmUmU*SmU*SmU | UU | OOSS |
| WV-47086 | mU*RfU*SmAmUn001SfAfGmAmGmCmAmAmGmAfAmCfAmCmU | UUAUAGAGCAAGAACACUGUU | RSOnSOOOOOOOOOOOOOO |
| | mGmUmU*SmU*SmU | UU | OOSS |
| WV-47087 | mU*RfU*SmAmUmAfGn001SfAmGmCmAmAmGmAfAmCfAmCmU | UUAUAGAGCAAGAACACUGUU | RSOOOnSOOOOOOOOOOOO |
| | mGmUmU*SmU*SmU | UU | OOSS |
| WV-47088 | mU*RfU*SmAmUmAfGmAn001SfGmCmAmAmGmAfAmCfAmCmU | UUAUAGAGCAAGAACACUGUU | RSOOOOnSOOOOOOOOOOO |
| | mGmUmU*SmU*SmU | UU | OOSS |
| WV-47089 | mU*RfU*SmAmUmAfGmAmGn001SfCmAmAmGmAfAmCfAmCmU | UUAUAGAGCAAGAACACUGUU | RSOOOOOnSOOOOOOOOOO |
| | mGmUmU*SmU*SmU | UU | OOSS |
| WV-47090 | mU*RfU*SmAmUmAfGmAmGmCn001SfAmAmGmAfAmCfAmCmU | UUAUAGAGCAAGAACACUGUU | RSOOOOOOnSOOOOOOOOO |
| | mGmUmU*SmU*SmU | UU | OOSS |
| WV-47091 | mU*RfU*SmAmUmAfGmAmGmCmAn001SfAmGmAfAmCfAmCmU | UUAUAGAGCAAGAACACUGUU | RSOOOOOOOnSOOOOOOOO |
| | mGmUmU*SmU*SmU | UU | OOSS |
| WV-47092 | mU*RfU*SmAmUmAfGmAmGmCmAmAn001SfGmAfAmCfAmCmU | UUAUAGAGCAAGAACACUGUU | RSOOOOOOOOnSOOOOOOO |
| | mGmUmU*SmU*SmU | UU | OOSS |
| WV-47093 | mU*RfU*SmAmUmAfGmAmGmCmAmAmGn001SfAfAmCfAmCmU | UUAUAGAGCAAGAACACUGUU | RSOOOOOOOOOnSOOOOOO |
| | mGmUmU*SmU*SmU | UU | OOSS |
| WV-47094 | mU*RfU*SmAmUmAfGmAmGmCmAmAmGmAfAn001SfCfAmCmU | UUAUAGAGCAAGAACACUGUU | RSOOOOOOOOOOOnSOOOO |
| | mGmUmU*SmU*SmU | UU | OOSS |
| WV-47095 | mU*RfU*SmAmUmAfGmAmGmCmAmAmGmAfAmCfAn001SfCmU | UUAUAGAGCAAGAACACUGUU | RSOOOOOOOOOOOOOnSOO |
| | mGmUmU*SmU*SmU | UU | OOSS |
| WV-47096 | mU*RfU*SmAmUmAfGmAmGmCmAmAmGmAfAmCfAmCn001SfU | UUAUAGAGCAAGAACACUGUU | RSOOOOOOOOOOOOOOnSO |
| | mGmUmU*SmU*SmU | UU | OOSS |
| WV-47097 | mU*RfU*SmAmUmAfGmAmGmCmAmAmGmAfAmCfAmCmUn001S | UUAUAGAGCAAGAACACUGUU | RSOOOOOOOOOOOOOOOnS |
| | fGmUmU*SmU*SmU | UU | OOSS |
| WV-47098 | mU*RfU*SmAmUmAfGmAmGmCmAmAmGmAfAmCfAmCmUmGn00 | UUAUAGAGCAAGAACACUGUU | RSOOOOOOOOOOOOOOOOn |
| | 1SfUmU*SmU*SmU | UU | SOSS |
| WV-47099 | mU*RfU*SmAmUmAfGmAmGmCmAmAmGmAfAmCfAmCmUmGmUn | UUAUAGAGCAAGAACACUGUU | RSOOOOOOOOOOOOOOOOO |
| | 001SfU*SmU*SmU | UU | nSSS |
| WV-47100 | mU*RfU*SmAmUmAfGmAmGmCmAmAmGmAfAmCfAmCmUmGmUm | UUAUAGAGCAAGAACACUGUU | RSOOOOOOOOOOOOOOOOO |
| | Un001SfU*SmU | UU | OnSS |
| WV-47101 | mU*RfU*SmAmUmAfGmAmGmCmAmAmGmAfAmCfAmCmUmGmUm | UUAUAGAGCAAGAACACUGUU | RSOOOOOOOOOOOOOOOOO |
| | U*SmUn001SfU | UU | OSnS |
| WV-43775 | mU*SfU*RmAmUmAfGmAmGmCmAmAmGmAfAmCfAmCmUmGmUm | UUAUAGAGCAAGAACACUGUU | SROOOOOOOOOOOOOOOOO |
| | U*SmU*SmU | UU | OSS |
| WV-47102 | mU*SfUn001RfAmUmAfGmAmGmCmAmAmGmAfAmCfAmCmUmG | UUAUAGAGCAAGAACACUGUU | SnROOOOOOOOOOOOOOOO |
| | mUmU*SmU*SmU | UU | OOSS |
| WV-47103 | mU*SfU*RmAn001RfUmAfGmAmGmCmAmAmGmAfAmCfAmCmU | UUAUAGAGCAAGAACACUGUU | SRnROOOOOOOOOOOOOOO |
| | mGmUmU*SmU*SmU | UU | OOSS |
| WV-47104 | mU*SfU*RmAmUn001RfAfGmAmGmCmAmAmGmAfAmCfAmCmU | UUAUAGAGCAAGAACACUGUU | SROnROOOOOOOOOOOOOO |
| | mGmUmU*SmU*SmU | UU | OOSS |
| WV-47105 | mU*SfU*RmAmUmAfGn001RfAmGmCmAmAmGmAfAmCfAmCmU | UUAUAGAGCAAGAACACUGUU | SROOOnROOOOOOOOOOOO |
| | mGmUmU*SmU*SmU | UU | OOSS |
| WV-47106 | mU*SfU*RmAmUmAfGmAn001RfGmCmAmAmGmAfAmCfAmCmU | UUAUAGAGCAAGAACACUGUU | SROOOOnROOOOOOOOOOO |
| | mGmUmU*SmU*SmU | UU | OOSS |
| WV-47107 | mU*SfU*RmAmUmAfGmAmGn001RfCmAmAmGmAfAmCfAmCmU | UUAUAGAGCAAGAACACUGUU | SROOOOOnROOOOOOOOOO |
| | mGmUmU*SmU*SmU | UU | OOSS |
| WV-47108 | mU*SfU*RmAmUmAfGmAmGmCn001RfAmAmGmAfAmCfAmCmU | UUAUAGAGCAAGAACACUGUU | SROOOOOOnROOOOOOOOO |
| | mGmUmU*SmU*SmU | UU | OOSS |
| WV-47109 | mU*SfU*RmAmUmAfGmAmGmCmAn001RfAmGmAfAmCfAmCmU | UUAUAGAGCAAGAACACUGUU | SROOOOOOOnROOOOOOOO |
| | mGmUmU*SmU*SmU | UU | OOSS |
| WV-47110 | mU*SfU*RmAmUmAfGmAmGmCmAmAn001RfGmAfAmCfAmCmU | UUAUAGAGCAAGAACACUGUU | SROOOOOOOOnROOOOOOO |
| | mGmUmU*SmU*SmU | UU | OOSS |
| WV-47111 | mU*SfU*RmAmUmAfGmAmGmCmAmAmGn001RfAfAmCfAmCmU | UUAUAGAGCAAGAACACUGUU | SROOOOOOOOOnROOOOOO |
| | mGmUmU*SmU*SmU | UU | OOSS |
| WV-47112 | mU*SfU*RmAmUmAfGmAmGmCmAmAmGmAfAn001RfCfAmCmU | UUAUAGAGCAAGAACACUGUU | SROOOOOOOOOOOnROOOO |
| | mGmUmU*SmU*SmU | UU | OOSS |
| WV-47113 | mU*SfU*RmAmUmAfGmAmGmCmAmAmGmAfAmCfAn001RfCmU | UUAUAGAGCAAGAACACUGUU | SROOOOOOOOOOOOOnROO |
| | mGmUmU*SmU*SmU | UU | OOSS |
| WV-47114 | mU*SfU*RmAmUmAfGmAmGmCmAmAmGmAfAmCfAmCn001RfU | UUAUAGAGCAAGAACACUGUU | SROOOOOOOOOOOOOOnRO |
| | mGmUmU*SmU*SmU | UU | OOSS |
| WV-47115 | mU*SfU*RmAmUmAfGmAmGmCmAmAmGmAfAmCfAmCmUn001R | UUAUAGAGCAAGAACACUGUU | SROOOOOOOOOOOOOOOnR |
| | fGmUmU*SmU*SmU | UU | OOSS |
| WV-47116 | mU*SfU*RmAmUmAfGmAmGmCmAmAmGmAfAmCfAmCmUmGn00 | UUAUAGAGCAAGAACACUGUU | SROOOOOOOOOOOOOOOOn |
| | 1RfUmU*SmU*SmU | UU | ROSS |
| WV-47118 | mU*SfU*RmAmUmAfGmAmGmCmAmAmGmAfAmCfAmCmUmGmUm | UUAUAGAGCAAGAACACUGUU | SROOOOOOOOOOOOOOOOO |
| | Un001RfU*SmU | UU | OnRS |
| WV-47119 | mU*SfU*RmAmUmAfGmAmGmCmAmAmGmAfAmCfAmCmUmGmUm | UUAUAGAGCAAGAACACUGUU | SROOOOOOOOOOOOOOOOO |
| | U*SmUn001RfU | UU | OSnR |
| WV-47120 | mU*SfUn001SfAmUmAfGmAmGmCmAmAmGmAfAmCfAmCmUmG | UUAUAGAGCAAGAACACUGUU | SnSOOOOOOOOOOOOOOOO |
| | mUmU*SmU*SmU | UU | OOSS |
| WV-47121 | mU*SfU*RmAn001SfUmAfGmAmGmCmAmAmGmAfAmCfAmCmU | UUAUAGAGCAAGAACACUGUU | SRnSOOOOOOOOOOOOOOO |
| | mGmUmU*SmU*SmU | UU | OOSS |
| WV-47122 | mU*SfU*RmAmUn001SfAfGmAmGmCmAmAmGmAfAmCfAmCmU | UUAUAGAGCAAGAACACUGUU | SROnSOOOOOOOOOOOOOO |
| | mGmUmU*SmU*SmU | UU | OOSS |
| WV-47123 | mU*SfU*RmAmUmAfGn001SfAmGmCmAmAmGmAfAmCfAmCmU | UUAUAGAGCAAGAACACUGUU | SROOOnSOOOOOOOOOOOO |
| | mGmUmU*SmU*SmU | UU | OOSS |
| WV-47124 | mU*SfU*RmAmUmAfGmAn001SfGmCmAmAmGmAfAmCfAmCmU | UUAUAGAGCAAGAACACUGUU | SROOOOnSOOOOOOOOOOO |
| | mGmUmU*SmU*SmU | UU | OOSS |
| WV-47125 | mU*SfU*RmAmUmAfGmAmGn001SfCmAmAmGmAfAmCfAmCmU | UUAUAGAGCAAGAACACUGUU | SROOOOOnSOOOOOOOOOO |
| | mGmUmU*SmU*SmU | UU | OOSS |
| WV-47126 | mU*SfU*RmAmUmAfGmAmGmCn001SfAmAmGmAfAmCfAmCmU | UUAUAGAGCAAGAACACUGUU | SROOOOOOnSOOOOOOOOO |
| | mGmUmU*SmU*SmU | UU | OOSS |
| WV-47127 | mU*SfU*RmAmUmAfGmAmGmCmAn001SfAmGmAfAmCfAmCmU | UUAUAGAGCAAGAACACUGUU | SROOOOOOOnSOOOOOOOO |
| | mGmUmU*SmU*SmU | UU | OOSS |
| WV-47128 | mU*SfU*RmAmUmAfGmAmGmCmAmAn001SfGmAfAmCfAmCmU | UUAUAGAGCAAGAACACUGUU | SROOOOOOOOnSOOOOOOO |
| | mGmUmU*SmU*SmU | UU | OOSS |
| WV-47129 | mU*SfU*RmAmUmAfGmAmGmCmAmAmGn001SfAfAmCfAmCmU | UUAUAGAGCAAGAACACUGUU | SROOOOOOOOOnSOOOOOO |
| | mGmUmU*SmU*SmU | UU | OOSS |
| WV-47130 | mU*SfU*RmAmUmAfGmAmGmCmAmAmGmAfAn001SfCfAmCmU | UUAUAGAGCAAGAACACUGUU | SROOOOOOOOOOOn$OOOO |
| | mGmUmU*SmU*SmU | UU | OOSS |
| WV-47131 | mU*SfU*RmAmUmAfGmAmGmCmAmAmGmAfAmCfAn001SfCmU | UUAUAGAGCAAGAACACUGUU | SROOOOOOOOOOOOOnSOO |
| | mGmUmU*SmU*SmU | UU | OOSS |
| WV-47132 | mU*SfU*RmAmUmAfGmAmGmCmAmAmGmAfAmCfAmCn001SfU | UUAUAGAGCAAGAACACUGUU | SROOOOOOOOOOOOOOnSO |
| | mGmUmU*SmU*SmU | UU | OOSS |
| WV-47133 | mU*SfU*RmAmUmAfGmAmGmCmAmAmGmAfAmCfAmCmUn001S | UUAUAGAGCAAGAACACUGUU | SROOOOOOOOOOOOOOOnS |
| | fGmUmU*SmU*SmU | UU | OOSS |
| WV-47134 | mU*SfU*RmAmUmAfGmAmGmCmAmAmGmAfAmCfAmCmUmGn00 | UUAUAGAGCAAGAACACUGUU | SROOOOOOOOOOOOOOOOn |
| | 1SfUmU*SmU*SmU | UU | SOSS |
| WV-47135 | mU*SfU*RmAmUmAfGmAmGmCmAmAmGmAfAmCfAmCmUmGmUn | UUAUAGAGCAAGAACACUGUU | SROOOOOOOOOOOOOOOOO |
| | 001SfU*SmU*SmU | UU | nSSS |
| WV-47136 | mU*SfU*RmAmUmAfGmAmGmCmAmAmGmAfAmCfAmCmUmGmUm | UUAUAGAGCAAGAACACUGUU | SROOOOOOOOOOOOOOOOO |
| | Un001SfU*SmU | UU | OnSS |
| WV-47137 | mU*SfU*RmAmUmAfGmAmGmCmAmAmGmAfAmCfAmCmUmGmUm | UUAUAGAGCAAGAACACUGUU | SROOOOOOOOOOOOOOOOO |
| | U*SmUn001SfU | UU | OSnS |
| WV-41826 | mU*fU*mAmUmAfGmAmGmCmAmAmGmAfAmCfAmCmUmGmUmU* | UUAUAGAGCAAGAACACUGUU | XXOOOOOOOOOOOOOOOOO |
| | mU*mU | UU | OXX |
| WV-41828 | Mod001L001mA*mAmCmAmGmUfGmUfUfCfUmUmGmCmUmCmU | AACAGUGUUCUUGCUCUAUAA | OXOOOOOOOOOOOOOOOOO |
| | mAmUmA*mA | | OX |
| WV-46380 | T*SfU*RmAmUmAfGmAmGmCmAmAmGmAfAmCfAmCmUmGmUmU | TUAUAGAGCAAGAACACUGUU | SROOOOOOOOOOOOOOOOO |
| | *SmU*SmU | UU | OSS |
| WV-46381 | POT*SfU*RmAmUmAfGmAmGmCmAmAmGmAfAmCfAmCmUmGmU | TUAUAGAGCAAGAACACUGUU | SROOOOOOOOOOOOOOOOO |
| | mU*SmU*SmU | UU | OSS |
| WV-46382 | PO5MSdT*SfU*RmAmUmAfGmAmGmCmAmAmGmAfAmCfAmCmU | TUAUAGAGCAAGAACACUGUU | SROOOOOOOOOOOOOOOOO |
| | mGmUmU*SmU*SmU | UU | OSS |
| WV-46383 | PO5MRdT*SfU*RmAmUmAfGmAmGmCmAmAmGmAfAmCfAmCmU | TUAUAGAGCAAGAACACUGUU | SROOOOOOOOOOOOOOOOO |
| | mGmUmU*SmU*SmU | UU | OSS |
| WV-46384 | VPT*SfU*RmAmUmAfGmAmGmCmAmAmGmAfAmCfAmCmUmGmU | TUAUAGAGCAAGAACACUGUU | SROOOOOOOOOOOOOOOOO |
| | mU*SmU*SmU | UU | OSS |
| WV-46385 | 5mspdT*SfU*RmAmUmAfGmAmGmCmAmAmGmAfAmCfAmCmUm | TUAUAGAGCAAGAACACUGUU | SROOOOOOOOOOOOOOOOO |
| | GmUmU*SmU*SmU | UU | OSS |
| WV-46386 | 5mrpdT*SfU*RmAmUmAfGmAmGmCmAmAmGmAfAmCfAmCmUm | TUAUAGAGCAAGAACACUGUU | SROOOOOOOOOOOOOOOOO |
| | GmUmU*SmU*SmU | UU | OSS |
| WV-42079 | mU*SfU*RmAn001SmUmAfGmAmGmCmAn001SmAmGmAfAmCf | UUAUAGAGCAAGAACACUGUU | SRnSOOOOOOnSOOOOOOO |
| | AmCmUmGn001SmUmU*SmU*SmU | UU | OnSOSS |
| WV-44434 | T*SfU*RmAn001SmUmAfGmAmGmCmAn001SmAmGmAfAmCfA | TUAUAGAGCAAGAACACUGUU | SRnSOOOOOOnSOOOOOOO |
| | mCmUmGn001SmUmU*SmU*SmU | UU | OnSOSS |
| WV-44435 | POT*SfU*RmAn001SmUmAfGmAmGmCmAn001SmAmGmAfAmC | TUAUAGAGCAAGAACACUGUU | SRnSOOOOOOnSOOOOOOO |
| | fAmCmUmGn001SmUmU*SmU*SmU | UU | OnsOSS |
| WV-44436 | PO5MSdT*SfU*RmAn001SmUmAfGmAmGmCmAn001SmAmGmA | TUAUAGAGCAAGAACACUGUU | SRnSOOOOOOnSOOOOOOO |
| | fAmCfAmCmUmGn001SmUmU*SmU*SmU | UU | OnSOSS |
| WV-44437 | PO5MRdT*SfU*RmAn001SmUmAfGmAmGmCmAn001SmAmGmA | TUAUAGAGCAAGAACACUGUU | SRnSOOOOOOnSOOOOOOO |
| | fAmCfAmCmUmGn001SmUmU*SmU*SmU | UU | OnSOSS |
| WV-44438 | VPT*SfU*RmAn001SmUmAfGmAmGmCmAn001SmAmGmAfAmC | TUAUAGAGCAAGAACACUGUU | SRnSOOOOOOnSOOOOOOO |
| | fAmCmUmGn001SmUmU*SmU*SmU | UU | OnSOSS |
| WV-44439 | 5mvpdT*SfU*RmAn001SmUmAfGmAmGmCmAn001SmAmGmAf | TUAUAGAGCAAGAACACUGUU | SRnSOOOOOOnSOOOOOOO |
| | AmCfAmCmUmGn001SmUmU*SmU*SmU | UU | OnSOSS |
| WV-44440 | 5mspdT*SfU*RmAn001SmUmAfGmAmGmCmAn001SmAmGmAf | TUAUAGAGCAAGAACACUGUU | SRnSOOOOOOnSOOOOOOO |
| | AmCfAmCmUmGn001SmUmU*SmU*SmU | UU | OnSOSS |
| WV-44441 | 5mrpdT*SfU*RmAn001SmUmAfGmAmGmCmAn001SmAmGmAf | TUAUAGAGCAAGAACACUGUU | SRnSOOOOOOnSOOOOOOO |
| | AmCfAmCmUmGn001SmUmU*SmU*SmU | UU | OnSOSS |
| WV-46387 | T*RfU*SmAmUmAfGmAmGmCmAmAmGmAfAmCfAmCmUmGmUmU | TUAUAGAGCAAGAACACUGUU | RSOOOOOOOOOOOOOOOOO |
| | *SmU*SmU | UU | OSS |
| WV-46388 | POT*RfU*SmAmUmAfGmAmGmCmAmAmGmAfAmCfAmCmUmGmU | TUAUAGAGCAAGAACACUGUU | RSOOOOOOOOOOOOOOOOO |
| | mU*SmU*SmU | UU | OSS |
| WV-46389 | PO5MSdT*RfU*SmAmUmAfGmAmGmCmAmAmGmAfAmCfAmCmU | TUAUAGAGCAAGAACACUGUU | RSOOOOOOOOOOOOOOOOO |
| | mGmUmU*SmU*SmU | UU | OSS |
| WV-46390 | PO5MRdT*RfU*SmAmUmAfGmAmGmCmAmAmGmAfAmCfAmCmU | TUAUAGAGCAAGAACACUGUU | RSOOOOOOOOOOOOOOOOO |
| | mGmUmU*SmU*SmU | UU | OSS |
| WV-46391 | VPT*RfU*SmAmUmAfGmAmGmCmAmAmGmAfAmCfAmCmUmGmU | TUAUAGAGCAAGAACACUGUU | RSOOOOOOOOOOOOOOOOO |
| | mU*SmU*SmU | UU | OSS |
| WV-46392 | 5mspdT*RfU*SmAmUmAfGmAmGmCmAmAmGmAfAmCfAmCmUm | TUAUAGAGCAAGAACACUGUU | RSOOOOOOOOOOOOOOOOO |
| | GmUmU*SmU*SmU | UU | OSS |
| WV-46393 | 5mrpdT*RfU*SmAmUmAfGmAmGmCmAmAmGmAfAmCfAmCmUm | TUAUAGAGCAAGAACACUGUU | RSOOOOOOOOOOOOOOOOO |
| | GmUmU*SmU*SmU | UU | OSS |
| WV-42078 | mU*RfU*SmAn001SmUmAfGmAmGmCmAn001SmAmGmAfAmCf | UUAUAGAGCAAGAACACUGUU | RSnSOOOOOOnSOOOOOOO |
| | AmCmUmGn001SmUmU*SmU*SmU | UU | OnSOSS |
| WV-46394 | T*RfU*SmAn001SmUmAfGmAmGmCmAn001SmAmGmAfAmCfA | TUAUAGAGCAAGAACACUGUU | RSnSOOOOOOnSOOOOOOO |
| | mCmUmGn001SmUmU*SmU*SmU | UU | OnSOSS |
| WV-46395 | POT*RfU*SmAn001SmUmAfGmAmGmCmAn001SmAmGmAfAmC | TUAUAGAGCAAGAACACUGUU | RSnSOOOOOOnSOOOOOOO |
| | fAmCmUmGn001SmUmU*SmU*SmU | UU | OnSOSS |
| WV-46396 | PO5MSdT*RfU*SmAn001SmUmAfGmAmGmCmAn001SmAmGmA | TUAUAGAGCAAGAACACUGUU | RSnSOOOOOOnSOOOOOOO |
| | fAmCfAmCmUmGn001SmUmU*SmU*SmU | UU | OnSOSS |
| WV-46397 | PO5MRdT*RfU*SmAn001SmUmAfGmAmGmCmAn001SmAmGmA | TUAUAGAGCAAGAACACUGUU | RSnSOOOOOOnSOOOOOOO |
| | fAmCfAmCmUmGn001SmUmU*SmU*SmU | UU | OnSOSS |
| WV-46398 | VPT*RfU*SmAn001SmUmAfGmAmGmCmAn001SmAmGmAfAmC | TUAUAGAGCAAGAACACUGUU | RSnSOOOOOOnSOOOOOOO |
| | fAmCmUmGn001SmUmU*SmU*SmU | UU | OnSOSS |
| WV-46399 | 5mspdT*RfU*SmAn001SmUmAfGmAmGmCmAn001SmAmGmAf | TUAUAGAGCAAGAACACUGUU | RSnSOOOOOOnSOOOOOOO |
| | AmCfAmCmUmGn001SmUmU*SmU*SmU | UU | OnSOSS |
| WV-46400 | 5mrpdT*RfU*SmAn001SmUmAfGmAmGmCmAn001SmAmGmAf | TUAUAGAGCAAGAACACUGUU | RSnSOOOOOOnSOOOOOOO |
| | AmCfAmCmUmGn001SmUmU*SmU*SmU | UU | OnSOSS |
| WV-42080 | Mod001L001mA*SmAmCmAmGmUfGmUfUfCfUmUmGmCmUmCm | AACAGUGUUCUUGCUCUAUAA | OSOOOOOOOOOOOOOOOOO |
| | UmAmUmA*SmA | | OS |
| WV-47144 | mU*RfU*SmAn001SfUmAfGmAmGmCmAn001SfAmGmAfAmCf | UUAUAGAGCAAGAACACUGUU | RSnSOOOOOOnSOOOOOOO |
| | AmCmUmGmUmU*SmU*SmU | UU | OOOSS |
| WV-47145 | mU*SfU*RmAn001SfUmAfGmAmGmCmAn001SfAmGmAfAmCf | UUAUAGAGCAAGAACACUGUU | SRnSOOOOOOnSOOOOOOO |
| | AmCmUmGmUmU*SmU*SmU | UU | OOOSS |
| WV-43771 | mU*SfU*RmAmUmAfGmAfGfCmAmAmGmAfAmCfAmCmUmGmUm | UUAUAGAGCAAGAACACUGUU | SROOOOOOOOOOOOOOOOO |
| | U*SmU*SmU | UU | OSS |
| WV-43773 | mU*SfU*RmAn001SmUmAfGmAfGfCmAn001SmAmGmAfAmCf | UUAUAGAGCAAGAACACUGUU | SRnSOOOOOOnSOOOOOOO |
| | AmCmUmGn001SmUmU*SmU*SmU | UU | OnSOSS |
| WV-43988 | mU*SfU*RmAn001SfUmAfGmAmGmCmAn001SfAmGmAfAmCf | UUAUAGAGCAAGAACACUGUU | SRnSOOOOOOnSOOOOOOO |
| | AmCmUmGn001SfUmU*SmU*SmU | UU | OnSOSS |
| WV-43989 | mU*SfU*RmAn001SfUmAfGmAmGmCfAn001SmAmGmAfAmCf | UUAUAGAGCAAGAACACUGUU | SRnSOOOOOOnSOOOOOOO |
| | AmCmUmGn001SfUmU*SmU*SmU | UU | OnSOSS |
| WV-43994 | mU*SfU*RfAn001SfUmAfGmAfGmCfAn001SfAmGmAfAmCf | UUAUAGAGCAAGAACACUGUU | SRnSOOOOOOnSOOOOOOO |
| | AmCfUfGn001SfUmU*SmU*SmU | UU | OnSOSS |
| WV-43996 | mU*SfU*RmAn001SfUmAfGmAfGfCmAn001SfAmGmAfAmCf | UUAUAGAGCAAGAACACUGUU | SRnSOOOOOOnSOOOOOOO |
| | AmCmUmGn001SfUmU*SmU*SmU | UU | OnSOSS |
| WV-43256 | mU*SfU*RmAn001SfUmAfGmAfGmCfAn001SmAmGmAfAmCf | UUAUAGAGCAAGAACACUGUU | SRnSOOOOOOnSOOOOOOO |
| | AmCfUmGn001SfUmU*SmU*SmU | UU | OnSOSS |
| WV-46991 | mU*fU*mAfUmAfGmAmGmCmAfAmGmAfAmCfAmCmUmGfUmU* | UUAUAGAGCAAGAACACUGUU | XXOOOOOOOOOOOOOOOOO |
| | mU*mU | UU | OXX |
| WV-46992 | mU*SfU*RmAfUmAfGmAmGmCmAfAmGmAfAmCfAmCmUmGfUm | UUAUAGAGCAAGAACACUGUU | SROOOOOOOOOOOOOOOOO |
| | U*SmU*SmU | UU | OSS |
| WV-46993 | T*SfU*RmAn001SfUmAfGmAmGmCmAn001SfAmGmAfAmCfA | TUAUAGAGCAAGAACACUGUU | SRnSOOOOOOnSOOOOOOO |
| | mCmUmGn001SfUmU*SmU*SmU | UU | OnSOSS |
| WV-46994 | POT*SfU*RmAn001SfUmAfGmAmGmCmAn001SfAmGmAfAmC | TUAUAGAGCAAGAACACUGUU | SRnSOOOOOOnSOOOOOOO |
| | fAmCmUmGn001SfUmU*SmU*SmU | UU | OnSOSS |
| WV-46995 | PO5MSdT*SfU*RmAn001SfUmAfGmAmGmCmAn001SfAmGmA | TUAUAGAGCAAGAACACUGUU | SRnSOOOOOOnSOOOOOOO |
| | fAmCfAmCmUmGn001SfUmU*SmU*SmU | UU | OnSOSS |
| WV-46996 | PO5MRdT*SfU*RmAn001SfUmAfGmAmGmCmAn001SfAmGmA | TUAUAGAGCAAGAACACUGUU | SRnSOOOOOOnSOOOOOOO |
| | fAmCfAmCmUmGn001SfUmU*SmU*SmU | UU | OnSOSS |
| WV-46997 | VPT*SfU*RmAn001SfUmAfGmAmGmCmAn001SfAmGmAfAmC | TUAUAGAGCAAGAACACUGUU | SRnSOOOOOOnSOOOOOOO |
| | fAmCmUmGn001SfUmU*SmU*SmU | UU | OnSOSS |
| WV-46998 | 5mspdT*SfU*RmAn001SfUmAfGmAmGmCmAn001SfAmGmAf | TUAUAGAGCAAGAACACUGUU | SRnSOOOOOOnSOOOOOOO |
| | AmCfAmCmUmGn001SfUmU*SmU*SmU | UU | OnSOSS |
| WV-46999 | 5mrpdT*SfU*RmAn001SfUmAfGmAmGmCmAn001SfAmGmAf | TUAUAGAGCAAGAACACUGUU | SRnSOOOOOOnSOOOOOOO |
| | AmCfAmCmUmGn001SfUmU*SmU*SmU | UU | OnSOSS |
| WV-47000 | mU*RfU*SmAfUmAfGmAmGmCmAfAmGmAfAmCfAmCmUmGfUm | UUAUAGAGCAAGAACACUGUU | RSOOOOOOOOOOOOOOOOO |
| | U*SmU*SmU | UU | OSS |
| WV-47001 | mU*RfU*SmAn001SfUmAfGmAmGmCmAn001SfAmGmAfAmCf | UUAUAGAGCAAGAACACUGUU | RSnSOOOOOOnSOOOOOOO |
| | AmCmUmGn001SfUmU*SmU*SmU | UU | OnSOSS |
| WV-47002 | T*RfU*SmAn001SfUmAfGmAmGmCmAn001SfAmGmAfAmCfA | TUAUAGAGCAAGAACACUGUU | RSnSOOOOOOnSOOOOOOO |
| | mCmUmGn001SfUmU*SmU*SmU | UU | OnSOSS |
| WV-47003 | POT*RfU*SmAn001SfUmAfGmAmGmCmAn001SfAmGmAfAmC | TUAUAGAGCAAGAACACUGUU | RSnSOOOOOOnSOOOOOOO |
| | fAmCmUmGn001SfUmU*SmU*SmU | UU | OnSOSS |
| WV-47004 | PO5MSdT*RfU*SmAn001SfUmAfGmAmGmCmAn001SfAmGmA | TUAUAGAGCAAGAACACUGUU | RSnSOOOOOOnSOOOOOOO |
| | fAmCfAmCmUmGn001SfUmU*SmU*SmU | UU | OnSOSS |
| WV-47005 | PO5MRdT*RfU*SmAn001SfUmAfGmAmGmCmAn001SfAmGmA | TUAUAGAGCAAGAACACUGUU | RSnSOOOOOOnSOOOOOOO |
| | fAmCfAmCmUmGn001SfUmU*SmU*SmU | UU | OnSOSS |
| WV-47006 | VPT*RfU*SmAn001SfUmAfGmAmGmCmAn001SfAmGmAfAmC | TUAUAGAGCAAGAACACUGUU | RSnSOOOOOOnSOOOOOOO |
| | fAmCmUmGn001SfUmU*SmU*SmU | UU | OnSOSS |
| WV-47007 | 5mspdT*RfU*SmAn001SfUmAfGmAmGmCmAn001SfAmGmAf | TUAUAGAGCAAGAACACUGUU | RSnSOOOOOOnSOOOOOOO |
| | AmCfAmCmUmGn001SfUmU*SmU*SmU | UU | OnSOSS |
| WV-47008 | 5mrpdT*RfU*SmAn001SfUmAfGmAmGmCmAn001SfAmGmAf | TUAUAGAGCAAGAACACUGUU | RSnSOOOOOOnSOOOOOOO |
| | AmCfAmCmUmGn001SfUmU*SmU*SmU | UU | OnSOSS |
| WV-47009 | mU*fU*mAfUmAfGmAfGfCmAfAmGmAfAmCfAmCmUmGfUmU* | UUAUAGAGCAAGAACACUGUU | XXOOOOOOOOOOOOOOOOO |
| | mU*mU | UU | OXX |
| WV-47010 | mU*SfU*RmAfUmAfGmAfGfCmAfAmGmAfAmCfAmCmUmGfUm | UUAUAGAGCAAGAACACUGUU | SROOOOOOOOOOOOOOOOO |
| | U*SmU*SmU | UU | OSS |
| WV-47011 | T*SfU*RmAn001SfUmAfGmAfGfCmAn001SfAmGmAfAmCfA | TUAUAGAGCAAGAACACUGUU | SRnSOOOOOOnSOOOOOOO |
| | mCmUmGn001SfUmU*SmU*SmU | UU | OnSOSS |
| WV-47012 | POT*SfU*RmAn001SfUmAfGmAfGfCmAn001SfAmGmAfAmC | TUAUAGAGCAAGAACACUGUU | SRnSOOOOOOnSOOOOOOO |
| | fAmCmUmGn001SfUmU*SmU*SmU | UU | OnSOSS |
| WV-47013 | PO5MSdT*SfU*RmAn001SfUmAfGmAfGfCmAn001SfAmGmA | TUAUAGAGCAAGAACACUGUU | SRnSOOOOOOnSOOOOOOO |
| | fAmCfAmCmUmGn001SfUmU*SmU*SmU | UU | OnSOSS |
| WV-47014 | PO5MRdT*SfU*RmAn001SfUmAfGmAfGfCmAn001SfAmGmA | TUAUAGAGCAAGAACACUGUU | SRnSOOOOOOnSOOOOOOO |
| | fAmCfAmCmUmGn001SfUmU*SmU*SmU | UU | OnSOSS |
| WV-47015 | VPT*SfU*RmAn001SfUmAfGmAfGfCmAn001SfAmGmAfAmC | TUAUAGAGCAAGAACACUGUU | SRnSOOOOOOnSOOOOOOO |
| | fAmCmUmGn001SfUmU*SmU*SmU | UU | OnSOSS |
| WV-47016 | 5mspdT*SfU*RmAn001SfUmAfGmAfGfCmAn001SfAmGmAf | TUAUAGAGCAAGAACACUGUU | SRnSOOOOOOnSOOOOOOO |
| | AmCfAmCmUmGn001SfUmU*SmU*SmU | UU | OnSOSS |
| WV-47017 | 5mrpdT*SfU*RmAn001SfUmAfGmAfGfCmAn001SfAmGmAf | TUAUAGAGCAAGAACACUGUU | SRnSOOOOOOnSOOOOOOO |
| | AmCfAmCmUmGn001SfUmU*SmU*SmU | UU | OnSOSS |
| WV-47018 | mU*RfU*SmAfUmAfGmAfGfCmAfAmGmAfAmCfAmCmUmGfUm | UUAUAGAGCAAGAACACUGUU | RSOOOOOOOOOOOOOOOOO |
| | U*SmU*SmU | UU | OSS |
| WV-47019 | mU*RfU*SmAn001SfUmAfGmAfGfCmAn001SfAmGmAfAmCf | UUAUAGAGCAAGAACACUGUU | RSnSOOOOOOnSOOOOOOO |
| | AmCmUmGn001SfUmU*SmU*SmU | UU | OnSOSS |
| WV-47020 | T*RfU*SmAn001SfUmAfGmAfGfCmAn001SfAmGmAfAmCfA | TUAUAGAGCAAGAACACUGUU | RSnSOOOOOOnSOOOOOOO |
| | mCmUmGn001SfUmU*SmU*SmU | UU | OnSOSS |
| WV-47021 | POT*RfU*SmAn001SfUmAfGmAfGfCmAn001SfAmGmAfAmC | TUAUAGAGCAAGAACACUGUU | RSnSOOOOOOnSOOOOOOO |
| | fAmCmUmGn001SfUmU*SmU*SmU | UU | OnSOSS |
| WV-47022 | PO5MSdT*RfU*SmAn001SfUmAfGmAfGfCmAn001SfAmGmA | TUAUAGAGCAAGAACACUGUU | RSnSOOOOOOnsOOOOOOO |
| | fAmCfAmCmUmGn001SfUmU*SmU*SmU | UU | OnSOSS |
| WV-47023 | PO5MRdT*RfU*SmAn001SfUmAfGmAfGfCmAn001SfAmGmA | TUAUAGAGCAAGAACACUGUU | RSnSOOOOOOnsOOOOOOO |
| | fAmCfAmCmUmGn001SfUmU*SmU*SmU | UU | OnSOSS |
| WV-47024 | VPT*RfU*SmAn001SfUmAfGmAfGfCmAn001SfAmGmAfAmC | TUAUAGAGCAAGAACACUGUU | RSnSOOOOOOnSOOOOOOO |
| | fAmCmUmGn001SfUmU*SmU*SmU | UU | OnSOSS |
| WV-47025 | 5mspdT*RfU*SmAn001SfUmAfGmAfGfCmAn001SfAmGmAf | TUAUAGAGCAAGAACACUGUU | RSnSOOOOOOnSOOOOOOO |
| | AmCfAmCmUmGn001SfUmU*SmU*SmU | UU | OnSOSS |
| WV-47026 | 5mrpdT*RfU*SmAn001SfUmAfGmAfGfCmAn001SfAmGmAf | TUAUAGAGCAAGAACACUGUU | RSnSOOOOOOnSOOOOOOO |
| | AmCfAmCmUmGn001SfUmU*SmU*SmU | UU | OnSOSS |
| WV-48526 | mU*RfU*SmAn001SfUmAfGmAfGmCfAn001RfAmGmAfAmCf | UUAUAGAGCAAGAACACUGUU | RSnSOOOOOOnROOOOOOO |
| | AmCfUmGn001RfUmU*SmU*SmU | UU | OnROSS |
| WV-48527 | mU*SfU*RmAn001SfUmAfGmAfGmCfAn001RfAmGmAfAmCf | UUAUAGAGCAAGAACACUGUU | SRnSOOOOOOnROOOOOOO |
| | AmCfUmGn001RfUmU*SmU*SmU | UU | OnROSS |
| WV-48528 | mU*SfU*RmAn001SfUmAfGmAmGmCmAn001RfAmGmAfAmCf | UUAUAGAGCAAGAACACUGUU | SRnSOOOOOOnROOOOOOO |
| | AmCmUmGmUmU*SmU*SmU | UU | OOOSS |
| WV-48529 | mU*SfU*RmAn001SfUmAfGmAmGmCmAn001RfAmGmAfAmCf | UUAUAGAGCAAGAACACUGUU | SRnSOOOOOOnROOOOOOO |
| | AmCmUmGn001RfUmU*SmU*SmU | UU | OnROSS |
| WV-48530 | mU*RfU*SmAmUmAfGmAmGmCmAn001RfAmGmAfAmCfAmCmU | UUAUAGAGCAAGAACACUGUU | RSOOOOOOOnROOOOOOOO |
| | mGn001RfUmU*SmU*SmU | UU | nROSS |
| WV-48531 | mU*SfU*RmAmUmAfGmAmGmCmAn001RfAmGmAfAmCfAmCmU | UUAUAGAGCAAGAACACUGUU | SROOOOOOOnROOOOOOOO |
| | mGn001RfUmU*SmU*SmU | UU | nROSS |
| WV-20167 | mU*fU*mAfUmAfGmAfGmCfAmAmGmAfAmCfAmCfUmGfUmU* | UUAUAGAGCAAGAACACUGUU | XXOOOOOOOOOOOOOOOOO |
| | mU*mU | UU | OXX |
| WV-20170 | mU*RfU*SmAfUmAfGmAfGmCfAmAmGmAfAmCfAmCfUmGfUm | UUAUAGAGCAAGAACACUGUU | RSOOOOOOOOOOOOOOOOO |
| | U*SmU*SmU | UU | OSS |
| WV-38708 | mU*RfU*SmAn001SfUmAfGmAfGmCfAn001SmAmGmAfAmCf | UUAUAGAGCAAGAACACUGUU | RSnSOOOOOOnSOOOOOOO |
| | AmCfUmGn001SfUmU*SmU*SmU | UU | OnSOSS |
| WV-36860 | Mod001L001mAmAfCmAfGmUfGmUfUfCfUmUfGmCfUmCfUm | AACAGUGUUCUUGCUCUAUAA | OOOOOOOOOOOOOOOOOOO |
| | AfUmAfA*mU*mU | UU | OOXX |
| WV-36807 | Mod001L001mAmAfCmAfGmUfGmUfUfCfUmUfGmCfUmCfUm | AACAGUGUUCUUGCUCUAUAA | OOOOOOOOOOOOOOOOOOO |
| | AfUmAfA*SmU*SmU | UU | OOSS |
| WV-20167 | mU*fU*mAfUmAfGmAfGmCfAmAmGmAfAmCfAmCfUmGfUmU* | UUAUAGAGCAAGAACACUGUU | XXOOOOOOOOOOOOOOOOO |
| | mU*mU | UU | OXX |
| WV-20169 | mU*SfU*SmAfUmAfGmAfGmCfAmAmGmAfAmCfAmCfUmGfUm | UUAUAGAGCAAGAACACUGUU | SSOOOOOOOOOOOOOOOOO |
| | U*SmU*SmU | UU | OSS |
| WV-20170 | mU*RfU*SmAfUmAfGmAfGmCfAmAmGmAfAmCfAmCfUmGfUm | UUAUAGAGCAAGAACACUGUU | RSOOOOOOOOOOOOOOOOO |
| | U*SmU*SmU | UU | OSS |
| WV-20171 | mU*SfU*RmAfUmAfGmAfGmCfAmAmGmAfAmCfAmCfUmGfUm | UUAUAGAGCAAGAACACUGUU | SROOOOOOOOOOOOOOOOO |
| | U*SmU*SmU | UU | OSS |
| WV-20172 | mU*RfU*RmAfUmAfGmAfGmCfAmAmGmAfAmCfAmCfUmGfUm | UUAUAGAGCAAGAACACUGUU | RROOOOOOOOOOOOOOOOO |
| | U*SmU*SmU | UU | OSS |
| WV-20183 | mU*SfU*RmAfUmAfGmAfGmCfAmAmGmAfAmCfAmCfUmGfUm | UUAUAGAGCAAGAACACUGUU | SROOOOOOOOOOOOOOOOO |
| | U*RmU*RmU | UU | ORR |
| WV-36836 | mUn001fU*mAfUmAfGmAfGmCfAmAmGmAfAmCfAmCfUmGfU | UUAUAGAGCAAGAACACUGUU | nXXOOOOOOOOOOOOOOOO |
| | mU*mU*mU | UU | OOXX |
| WV-36837 | mU*fUn001mAfUmAfGmAfGmCfAmAmGmAfAmCfAmCfUmGfU | UUAUAGAGCAAGAACACUGUU | XnXOOOOOOOOOOOOOOOO |
| | mU*mU*mU | UU | OOXX |
| WV-36838 | mU*fU*mAn001fUmAfGmAfGmCfAmAmGmAfAmCfAmCfUmGf | UUAUAGAGCAAGAACACUGUU | XXnXOOOOOOOOOOOOOOO |
| | UmU*mU*mU | UU | OOXX |
| WV-36839 | mU*fU*mAfUn001mAfGmAfGmCfAmAmGmAfAmCfAmCfUmGf | UUAUAGAGCAAGAACACUGUU | XXOnXOOOOOOOOOOOOOO |
| | UmU*mU*mU | UU | OOXX |
| WV-36840 | mU*fU*mAfUmAn001fGmAfGmCfAmAmGmAfAmCfAmCfUmGf | UUAUAGAGCAAGAACACUGUU | XXOOnXOOOOOOOOOOOOO |
| | UmU*mU*mU | UU | OOXX |
| WV-36841 | mU*fU*mAfUmAfGn001mAfGmCfAmAmGmAfAmCfAmCfUmGf | UUAUAGAGCAAGAACACUGUU | XXOOOnXOOOOOOOOOOOO |
| | UmU*mU*mU | UU | OOXX |
| WV-36842 | mU*fU*mAfUmAfGmAn001fGmCfAmAmGmAfAmCfAmCfUmGf | UUAUAGAGCAAGAACACUGUU | XXOOOOnXOOOOOOOOOOO |
| | UmU*mU*mU | UU | OOXX |
| WV-36843 | mU*fU*mAfUmAfGmAfGn001mCfAmAmGmAfAmCfAmCfUmGf | UUAUAGAGCAAGAACACUGUU | XXOOOOOnXOOOOOOOOOO |
| | UmU*mU*mU | UU | OOXX |
| WV-36844 | mU*fU*mAfUmAfGmAfGmCn001fAmAmGmAfAmCfAmCfUmGf | UUAUAGAGCAAGAACACUGUU | XXOOOOOOnXOOOOOOOOO |
| | UmU*mU*mU | UU | OOXX |
| WV-36845 | mU*fU*mAfUmAfGmAfGmCfAn001mAmGmAfAmCfAmCfUmGf | UUAUAGAGCAAGAACACUGUU | XXOOOOOOOnXOOOOOOOO |
| | UmU*mU*mU | UU | OOXX |
| WV-36846 | mU*fU*mAfUmAfGmAfGmCfAmAn001mGmAfAmCfAmCfUmGf | UUAUAGAGCAAGAACACUGUU | XXOOOOOOOOnxOOOOOOO |
| | UmU*mU*mU | UU | OOXX |
| WV-36847 | mU*fU*mAfUmAfGmAfGmCfAmAmGn001mAfAmCfAmCfUmGf | UUAUAGAGCAAGAACACUGUU | XXOOOOOOOOOnxOOOOOO |
| | UmU*mU*mU | UU | OOXX |
| WV-36848 | mU*fU*mAfUmAfGmAfGmCfAmAmGmAn001fAmCfAmCfUmGf | UUAUAGAGCAAGAACACUGUU | XXOOOOOOOOOOnXOOOOO |
| | UmU*mU*mU | UU | OOXX |
| WV-36849 | mU*fU*mAfUmAfGmAfGmCfAmAmGmAfAn001mCfAmCfUmGf | UUAUAGAGCAAGAACACUGUU | XXOOOOOOOOOOOnXOOOO |
| | UmU*mU*mU | UU | OOXX |
| WV-36850 | mU*fU*mAfUmAfGmAfGmCfAmAmGmAfAmCn001fAmCfUmGf | UUAUAGAGCAAGAACACUGUU | XXOOOOOOOOOOOOnxOOO |
| | UmU*mU*mU | UU | OOXX |
| WV-36851 | mU*fU*mAfUmAfGmAfGmCfAmAmGmAfAmCfAn001mCfUmGf | UUAUAGAGCAAGAACACUGUU | XXOOOOOOOOOOOOOnXOO |
| | UmU*mU*mU | UU | OOXX |
| WV-36852 | mU*fU*mAfUmAfGmAfGmCfAmAmGmAfAmCfAmCn001fUmGf | UUAUAGAGCAAGAACACUGUU | XXOOOOOOOOOOOOOOnXO |
| | UmU*mU*mU | UU | OOXX |
| WV-36853 | mU*fU*mAfUmAfGmAfGmCfAmAmGmAfAmCfAmCfUn001mGf | UUAUAGAGCAAGAACACUGUU | XXOOOOOOOOOOOOOOOnX |
| | UmU*mU*mU | UU | OOXX |
| WV-36854 | mU*fU*mAfUmAfGmAfGmCfAmAmGmAfAmCfAmCfUmGn001f | UUAUAGAGCAAGAACACUGUU | XXOOOOOOOOOOOOOOOOn |
| | UmU*mU*mU | UU | XOXX |
| WV-36855 | mU*fU*mAfUmAfGmAfGmCfAmAmGmAfAmCfAmCfUmGfUn00 | UUAUAGAGCAAGAACACUGUU | XXOOOOOOOOOOOOOOOOO |
| | 1mU*mU*mU | UU | nXXX |
| WV-36856 | mU*fU*mAfUmAfGmAfGmCfAmAmGmAfAmCfAmCfUmGfUmUn | UUAUAGAGCAAGAACACUGUU | XXOOOOOOOOOOOOOOOOO |
| | 001mU*mU | UU | OnXX |
| WV-36857 | mU*fU*mAfUmAfGmAfGmCfAmAmGmAfAmCfAmCfUmGfUmU* | UUAUAGAGCAAGAACACUGUU | XXOOOOOOOOOOOOOOOOO |
| | mUn001mU | UU | OXnX |
| WV-36980 | mU*RfU*RmA*RfU*RmA*RfG*RmA*RfG*SmCfAmAmGmAfAm | UUAUAGAGCAAGAACACUGUU | RRRRRRRSOOOOOOOOOOO |
| | CfAmCfUmGfUmU*SmU*SmU | UU | OSS |
| WV-36981 | mU*RfU*RmA*RfU*RmA*RfG*RmA*SfG*RmCfAmAmGmAfAm | UUAUAGAGCAAGAACACUGUU | RRRRRRSROOOOOOOOOOO |
| | CfAmCfUmGfUmU*SmU*SmU | UU | OSS |
| WV-36982 | mU*RfU*RmA*RfU*RmA*RfG*RmA*SfG*SmCfAmAmGmAfAm | UUAUAGAGCAAGAACACUGUU | RRRRRRSSOOOOOOOOOOO |
| | CfAmCfUmGfUmU*SmU*SmU | UU | OSS |
| WV-36983 | mU*RfU*RmA*RfU*RmA*RfG*SmA*RfG*RmCfAmAmGmAfAm | UUAUAGAGCAAGAACACUGUU | RRRRRSRROOOOOOOOOOO |
| | CfAmCfUmGfUmU*SmU*SmU | UU | OSS |
| WV-36984 | mU*RfU*RmA*RfU*RmA*RfG*SmA*RfG*SmCfAmAmGmAfAm | UUAUAGAGCAAGAACACUGUU | RRRRRSRSOOOOOOOOOOO |
| | CfAmCfUmGfUmU*SmU*SmU | UU | OSS |
| WV-36985 | mU*RfU*RmA*RfU*RmA*RfG*SmA*SfG*RmCfAmAmGmAfAm | UUAUAGAGCAAGAACACUGUU | RRRRRSSROOOOOOOOOOO |
| | CfAmCfUmGfUmU*SmU*SmU | UU | OSS |
| WV-36986 | mU*RfU*RmA*RfU*RmA*RfG*SmA*SfG*SmCfAmAmGmAfAm | UUAUAGAGCAAGAACACUGUU | RRRRRSSSOOOOOOOOOOO |
| | CfAmCfUmGfUmU*SmU*SmU | UU | OSS |
| WV-36987 | mU*RfU*RmA*RfU*RmA*SfG*RmA*RfG*RmCfAmAmGmAfAm | UUAUAGAGCAAGAACACUGUU | RRRRSRRROOOOOOOOOOO |
| | CfAmCfUmGfUmU*SmU*SmU | UU | OSS |
| WV-36988 | mU*RfU*RmA*RfU*RmA*SfG*RmA*RfG*SmCfAmAmGmAfAm | UUAUAGAGCAAGAACACUGUU | RRRRSRRSOOOOOOOOOOO |
| | CfAmCfUmGfUmU*SmU*SmU | UU | OSS |
| WV-36989 | mU*RfU*RmA*RfU*RmA*SfG*RmA*SfG*RmCfAmAmGmAfAm | UUAUAGAGCAAGAACACUGUU | RRRRSRSROOOOOOOOOOO |
| | CfAmCfUmGfUmU*SmU*SmU | UU | OSS |
| WV-36990 | mU*RfU*RmA*RfU*RmA*SfG*RmA*SfG*SmCfAmAmGmAfAm | UUAUAGAGCAAGAACACUGUU | RRRRSRSSOOOOOOOOOOO |
| | CfAmCfUmGfUmU*SmU*SmU | UU | OSS |
| WV-36991 | mU*RfU*RmA*RfU*RmA*SfG*SmA*RfG*RmCfAmAmGmAfAm | UUAUAGAGCAAGAACACUGUU | RRRRSSRROOOOOOOOOOO |
| | CfAmCfUmGfUmU*SmU*SmU | UU | OSS |
| WV-36992 | mU*RfU*RmA*RfU*RmA*SfG*SmA*RfG*SmCfAmAmGmAfAm | UUAUAGAGCAAGAACACUGUU | RRRRSSRSOOOOOOOOOOO |
| | CfAmCfUmGfUmU*SmU*SmU | UU | OSS |
| WV-36993 | mU*RfU*RmA*RfU*RmA*SfG*SmA*SfG*RmCfAmAmGmAfAm | UUAUAGAGCAAGAACACUGUU | RRRRSSSROOOOOOOOOOO |
| | CfAmCfUmGfUmU*SmU*SmU | UU | OSS |
| WV-36994 | mU*RfU*RmA*RfU*RmA*SfG*SmA*SfG*SmCfAmAmGmAfAm | UUAUAGAGCAAGAACACUGUU | RRRRSSSSOOOOOOOOOOO |
| | CfAmCfUmGfUmU*SmU*SmU | UU | OSS |
| WV-36995 | mU*RfU*RmA*RfU*SmA*RfG*RmA*RfG*RmCfAmAmGmAfAm | UUAUAGAGCAAGAACACUGUU | RRRSRRRROOOOOOOOOOO |
| | CfAmCfUmGfUmU*SmU*SmU | UU | OSS |
| WV-36996 | mU*RfU*RmA*RfU*SmA*RfG*RmA*RfG*SmCfAmAmGmAfAm | UUAUAGAGCAAGAACACUGUU | RRRSRRRSOOOOOOOOOOO |
| | CfAmCfUmGfUmU*SmU*SmU | UU | OSS |
| WV-36997 | mU*RfU*RmA*RfU*SmA*RfG*RmA*SfG*RmCfAmAmGmAfAm | UUAUAGAGCAAGAACACUGUU | RRRSRRSROOOOOOOOOOO |
| | CfAmCfUmGfUmU*SmU*SmU | UU | OSS |
| WV-36998 | mU*RfU*RmA*RfU*SmA*RfG*RmA*SfG*SmCfAmAmGmAfAm | UUAUAGAGCAAGAACACUGUU | RRRSRRSSOOOOOOOOOOO |
| | CfAmCfUmGfUmU*SmU*SmU | UU | OSS |
| WV-36999 | mU*RfU*RmA*RfU*SmA*RfG*SmA*RfG*RmCfAmAmGmAfAm | UUAUAGAGCAAGAACACUGUU | RRRSRSRROOOOOOOOOOO |
| | CfAmCfUmGfUmU*SmU*SmU | UU | OSS |
| WV-37000 | mU*RfU*RmA*RfU*SmA*RfG*SmA*RfG*SmCfAmAmGmAfAm | UUAUAGAGCAAGAACACUGUU | RRRSRSRSOOOOOOOOOOO |
| | CfAmCfUmGfUmU*SmU*SmU | UU | OSS |
| WV-37001 | mU*RfU*RmA*RfU*SmA*RfG*SmA*SfG*RmCfAmAmGmAfAm | UUAUAGAGCAAGAACACUGUU | RRRSRSSROOOOOOOOOOO |
| | CfAmCfUmGfUmU*SmU*SmU | UU | OSS |
| WV-37002 | mU*RfU*RmA*RfU*SmA*RfG*SmA*SfG*SmCfAmAmGmAfAm | UUAUAGAGCAAGAACACUGUU | RRRSRSSSOOOOOOOOOOO |
| | CfAmCfUmGfUmU*SmU*SmU | UU | OSS |
| WV-37003 | mU*RfU*RmA*RfU*SmA*SfG*RmA*RfG*RmCfAmAmGmAfAm | UUAUAGAGCAAGAACACUGUU | RRRSSRRROOOOOOOOOOO |
| | CfAmCfUmGfUmU*SmU*SmU | UU | OSS |
| WV-37004 | mU*RfU*RmA*RfU*SmA*SfG*RmA*RfG*SmCfAmAmGmAfAm | UUAUAGAGCAAGAACACUGUU | RRRSSRRSOOOOOOOOOOO |
| | CfAmCfUmGfUmU*SmU*SmU | UU | OSS |
| WV-37005 | mU*RfU*RmA*RfU*SmA*SfG*RmA*SfG*RmCfAmAmGmAfAm | UUAUAGAGCAAGAACACUGUU | RRRSSRSROOOOOOOOOOO |
| | CfAmCfUmGfUmU*SmU*SmU | UU | OSS |
| WV-37006 | mU*RfU*RmA*RfU*SmA*SfG*RmA*SfG*SmCfAmAmGmAfAm | UUAUAGAGCAAGAACACUGUU | RRRSSRSSOOOOOOOOOOO |
| | CfAmCfUmGfUmU*SmU*SmU | UU | OSS |
| WV-37007 | mU*RfU*RmA*RfU*SmA*SfG*SmA*RfG*RmCfAmAmGmAfAm | UUAUAGAGCAAGAACACUGUU | RRRSSSRROOOOOOOOOOO |
| | CfAmCfUmGfUmU*SmU*SmU | UU | OSS |
| WV-37008 | mU*RfU*RmA*RfU*SmA*SfG*SmA*RfG*SmCfAmAmGmAfAm | UUAUAGAGCAAGAACACUGUU | RRRSSSRSOOOOOOOOOOO |
| | CfAmCfUmGfUmU*SmU*SmU | UU | OSS |
| WV-37009 | mU*RfU*RmA*RfU*SmA*SfG*SmA*SfG*RmCfAmAmGmAfAm | UUAUAGAGCAAGAACACUGUU | RRRSSSSROOOOOOOOOOO |
| | CfAmCfUmGfUmU*SmU*SmU | UU | OSS |
| WV-37010 | mU*RfU*RmA*RfU*SmA*SfG*SmA*SfG*SmCfAmAmGmAfAm | UUAUAGAGCAAGAACACUGUU | RRRSSSSSOOOOOOOOOOO |
| | CfAmCfUmGfUmU*SmU*SmU | UU | OSS |
| WV-37011 | mU*RfU*RmA*SfU*RmA*RfG*RmA*RfG*RmCfAmAmGmAfAm | UUAUAGAGCAAGAACACUGUU | RRSRRRRROOOOOOOOOOO |
| | CfAmCfUmGfUmU*SmU*SmU | UU | OSS |
| WV-37012 | mU*RfU*RmA*SfU*RmA*RfG*RmA*RfG*SmCfAmAmGmAfAm | UUAUAGAGCAAGAACACUGUU | RRSRRRRSOOOOOOOOOOO |
| | CfAmCfUmGfUmU*SmU*SmU | UU | OSS |
| WV-37013 | mU*RfU*RmA*SfU*RmA*RfG*RmA*SfG*RmCfAmAmGmAfAm | UUAUAGAGCAAGAACACUGUU | RRSRRRSROOOOOOOOOOO |
| | CfAmCfUmGfUmU*SmU*SmU | UU | OSS |
| WV-37014 | mU*RfU*RmA*SfU*RmA*RfG*RmA*SfG*SmCfAmAmGmAfAm | UUAUAGAGCAAGAACACUGUU | RRSRRRSSOOOOOOOOOOO |
| | CfAmCfUmGfUmU*SmU*SmU | UU | OSS |
| WV-37015 | mU*RfU*RmA*SfU*RmA*RfG*SmA*RfG*RmCfAmAmGmAfAm | UUAUAGAGCAAGAACACUGUU | RRSRRSRROOOOOOOOOOO |
| | CfAmCfUmGfUmU*SmU*SmU | UU | OSS |
| WV-37016 | mU*RfU*RmA*SfU*RmA*RfG*SmA*RfG*SmCfAmAmGmAfAm | UUAUAGAGCAAGAACACUGUU | RRSRRSRSOOOOOOOOOOO |
| | CfAmCfUmGfUmU*SmU*SmU | UU | OSS |
| WV-37017 | mU*RfU*RmA*SfU*RmA*RfG*SmA*SfG*RmCfAmAmGmAfAm | UUAUAGAGCAAGAACACUGUU | RRSRRSSROOOOOOOOOOO |
| | CfAmCfUmGfUmU*SmU*SmU | UU | OSS |
| WV-37018 | mU*RfU*RmA*SfU*RmA*RfG*SmA*SfG*SmCfAmAmGmAfAm | UUAUAGAGCAAGAACACUGUU | RRSRRSSSOOOOOOOOOOO |
| | CfAmCfUmGfUmU*SmU*SmU | UU | OSS |
| WV-37019 | mU*RfU*RmA*SfU*RmA*SfG*RmA*RfG*RmCfAmAmGmAfAm | UUAUAGAGCAAGAACACUGUU | RRSRSRRROOOOOOOOOOO |
| | CfAmCfUmGfUmU*SmU*SmU | UU | OSS |
| WV-37020 | mU*RfU*RmA*SfU*RmA*SfG*RmA*RfG*SmCfAmAmGmAfAm | UUAUAGAGCAAGAACACUGUU | RRSRSRRSOOOOOOOOOOO |
| | CfAmCfUmGfUmU*SmU*SmU | UU | OSS |
| WV-37021 | mU*RfU*RmA*SfU*RmA*SfG*RmA*SfG*RmCfAmAmGmAfAm | UUAUAGAGCAAGAACACUGUU | RRSRSRSROOOOOOOOOOO |
| | CfAmCfUmGfUmU*SmU*SmU | UU | OSS |
| WV-37022 | mU*RfU*RmA*SfU*RmA*SfG*RmA*SfG*SmCfAmAmGmAfAm | UUAUAGAGCAAGAACACUGUU | RRSRSRSSOOOOOOOOOOO |
| | CfAmCfUmGfUmU*SmU*SmU | UU | OSS |
| WV-37023 | mU*RfU*RmA*SfU*RmA*SfG*SmA*RfG*RmCfAmAmGmAfAm | UUAUAGAGCAAGAACACUGUU | RRSRSSRROOOOOOOOOOO |
| | CfAmCfUmGfUmU*SmU*SmU | UU | OSS |
| WV-37024 | mU*RfU*RmA*SfU*RmA*SfG*SmA*RfG*SmCfAmAmGmAfAm | UUAUAGAGCAAGAACACUGUU | RRSRSSRSOOOOOOOOOOO |
| | CfAmCfUmGfUmU*SmU*SmU | UU | OSS |
| WV-37025 | mU*RfU*RmA*SfU*RmA*SfG*SmA*SfG*RmCfAmAmGmAfAm | UUAUAGAGCAAGAACACUGUU | RRSRSSSROOOOOOOOOOO |
| | CfAmCfUmGfUmU*SmU*SmU | UU | OSS |
| WV-37026 | mU*RfU*RmA*SfU*RmA*SfG*SmA*SfG*SmCfAmAmGmAfAm | UUAUAGAGCAAGAACACUGUU | RRSRSSSSOOOOOOOOOOO |
| | CfAmCfUmGfUmU*SmU*SmU | UU | OSS |
| WV-37027 | mU*RfU*RmA*SfU*SmA*RfG*RmA*RfG*RmCfAmAmGmAfAm | UUAUAGAGCAAGAACACUGUU | RRSSRRRROOOOOOOOOOO |
| | CfAmCfUmGfUmU*SmU*SmU | UU | OSS |
| WV-37028 | mU*RfU*RmA*SfU*SmA*RfG*RmA*RfG*SmCfAmAmGmAfAm | UUAUAGAGCAAGAACACUGUU | RRSSRRRSOOOOOOOOOOO |
| | CfAmCfUmGfUmU*SmU*SmU | UU | OSS |
| WV-37029 | mU*RfU*RmA*SfU*SmA*RfG*RmA*SfG*RmCfAmAmGmAfAm | UUAUAGAGCAAGAACACUGUU | RRSSRRSROOOOOOOOOOO |
| | CfAmCfUmGfUmU*SmU*SmU | UU | OSS |
| WV-37030 | mU*RfU*RmA*SfU*SmA*RfG*RmA*SfG*SmCfAmAmGmAfAm | UUAUAGAGCAAGAACACUGUU | RRSSRRSSOOOOOOOOOOO |
| | CfAmCfUmGfUmU*SmU*SmU | UU | OSS |
| WV-37031 | mU*RfU*RmA*SfU*SmA*RfG*SmA*RfG*RmCfAmAmGmAfAm | UUAUAGAGCAAGAACACUGUU | RRSSRSRROOOOOOOOOOO |
| | CfAmCfUmGfUmU*SmU*SmU | UU | OSS |
| WV-37032 | mU*RfU*RmA*SfU*SmA*RfG*SmA*RfG*SmCfAmAmGmAfAm | UUAUAGAGCAAGAACACUGUU | RRSSRSRSOOOOOOOOOOO |
| | CfAmCfUmGfUmU*SmU*SmU | UU | OSS |
| WV-37033 | mU*RfU*RmA*SfU*SmA*RfG*SmA*SfG*RmCfAmAmGmAfAm | UUAUAGAGCAAGAACACUGUU | RRSSRSSROOOOOOOOOOO |
| | CfAmCfUmGfUmU*SmU*SmU | UU | OSS |
| WV-37034 | mU*RfU*RmA*SfU*SmA*RfG*SmA*SfG*SmCfAmAmGmAfAm | UUAUAGAGCAAGAACACUGUU | RRSSRSSSOOOOOOOOOOO |
| | CfAmCfUmGfUmU*SmU*SmU | UU | OSS |
| WV-37035 | mU*RfU*RmA*SfU*SmA*SfG*RmA*RfG*RmCfAmAmGmAfAm | UUAUAGAGCAAGAACACUGUU | RRSSSRRROOOOOOOOOOO |
| | CfAmCfUmGfUmU*SmU*SmU | UU | OSS |
| WV-37036 | mU*RfU*RmA*SfU*SmA*SfG*RmA*RfG*SmCfAmAmGmAfAm | UUAUAGAGCAAGAACACUGUU | RRSSSRRSOOOOOOOOOOO |
| | CfAmCfUmGfUmU*SmU*SmU | UU | OSS |
| WV-37037 | mU*RfU*RmA*SfU*SmA*SfG*RmA*SfG*RmCfAmAmGmAfAm | UUAUAGAGCAAGAACACUGUU | RRSSSRSROOOOOOOOOOO |
| | CfAmCfUmGfUmU*SmU*SmU | UU | OSS |
| WV-37038 | mU*RfU*RmA*SfU*SmA*SfG*RmA*SfG*SmCfAmAmGmAfAm | UUAUAGAGCAAGAACACUGUU | RRSSSRSSOOOOOOOOOOO |
| | CfAmCfUmGfUmU*SmU*SmU | UU | OSS |
| WV-37039 | mU*RfU*RmA*SfU*SmA*SfG*SmA*RfG*RmCfAmAmGmAfAm | UUAUAGAGCAAGAACACUGUU | RRSSSSRROOOOOOOOOOO |
| | CfAmCfUmGfUmU*SmU*SmU | UU | OSS |
| WV-37040 | mU*RfU*RmA*SfU*SmA*SfG*SmA*RfG*SmCfAmAmGmAfAm | UUAUAGAGCAAGAACACUGUU | RRSSSSRSOOOOOOOOOOO |
| | CfAmCfUmGfUmU*SmU*SmU | UU | OSS |
| WV-37041 | mU*RfU*RmA*SfU*SmA*SfG*SmA*SfG*RmCfAmAmGmAfAm | UUAUAGAGCAAGAACACUGUU | RRSSSSSROOOOOOOOOOO |
| | CfAmCfUmGfUmU*SmU*SmU | UU | OSS |
| WV-37042 | mU*RfU*RmA*SfU*SmA*SfG*SmA*SfG*SmCfAmAmGmAfAm | UUAUAGAGCAAGAACACUGUU | RRSSSSSSOOOOOOOOOOO |
| | CfAmCfUmGfUmU*SmU*SmU | UU | OSS |
| WV-37043 | mU*RfU*SmA*RfU*RmA*RfG*RmA*RfG*RmCfAmAmGmAfAm | UUAUAGAGCAAGAACACUGUU | RSRRRRRROOOOOOOOOOO |
| | CfAmCfUmGfUmU*SmU*SmU | UU | OSS |
| WV-37044 | mU*RfU*SmA*RfU*RmA*RfG*RmA*RfG*SmCfAmAmGmAfAm | UUAUAGAGCAAGAACACUGUU | RSRRRRRSOOOOOOOOOOO |
| | CfAmCfUmGfUmU*SmU*SmU | UU | OSS |
| WV-37045 | mU*RfU*SmA*RfU*RmA*RfG*RmA*SfG*RmCfAmAmGmAfAm | UUAUAGAGCAAGAACACUGUU | RSRRRRSROOOOOOOOOOO |
| | CfAmCfUmGfUmU*SmU*SmU | UU | OSS |
| WV-37046 | mU*RfU*SmA*RfU*RmA*RfG*RmA*SfG*SmCfAmAmGmAfAm | UUAUAGAGCAAGAACACUGUU | RSRRRRSSOOOOOOOOOOO |
| | CfAmCfUmGfUmU*SmU*SmU | UU | OSS |
| WV-37047 | mU*RfU*SmA*RfU*RmA*RfG*SmA*RfG*RmCfAmAmGmAfAm | UUAUAGAGCAAGAACACUGUU | RSRRRSRROOOOOOOOOOO |
| | CfAmCfUmGfUmU*SmU*SmU | UU | OSS |
| WV-37048 | mU*RfU*SmA*RfU*RmA*RfG*SmA*RfG*SmCfAmAmGmAfAm | UUAUAGAGCAAGAACACUGUU | RSRRRSRSOOOOOOOOOOO |
| | CfAmCfUmGfUmU*SmU*SmU | UU | OSS |
| WV-37049 | mU*RfU*SmA*RfU*RmA*RfG*SmA*SfG*RmCfAmAmGmAfAm | UUAUAGAGCAAGAACACUGUU | RSRRRSSROOOOOOOOOOO |
| | CfAmCfUmGfUmU*SmU*SmU | UU | OSS |
| WV-37050 | mU*RfU*SmA*RfU*RmA*RfG*SmA*SfG*SmCfAmAmGmAfAm | UUAUAGAGCAAGAACACUGUU | RSRRRSSSOOOOOOOOOOO |
| | CfAmCfUmGfUmU*SmU*SmU | UU | OSS |
| WV-37051 | mU*RfU*SmA*RfU*RmA*SfG*RmA*RfG*RmCfAmAmGmAfAm | UUAUAGAGCAAGAACACUGUU | RSRRSRRROOOOOOOOOOO |
| | CfAmCfUmGfUmU*SmU*SmU | UU | OSS |
| WV-37052 | mU*RfU*SmA*RfU*RmA*SfG*RmA*RfG*SmCfAmAmGmAfAm | UUAUAGAGCAAGAACACUGUU | RSRRSRRSOOOOOOOOOOO |
| | CfAmCfUmGfUmU*SmU*SmU | UU | OSS |
| WV-37053 | mU*RfU*SmA*RfU*RmA*SfG*RmA*SfG*RmCfAmAmGmAfAm | UUAUAGAGCAAGAACACUGUU | RSRRSRSROOOOOOOOOOO |
| | CfAmCfUmGfUmU*SmU*SmU | UU | OSS |
| WV-37054 | mU*RfU*SmA*RfU*RmA*SfG*RmA*SfG*SmCfAmAmGmAfAm | UUAUAGAGCAAGAACACUGUU | RSRRSRSSOOOOOOOOOOO |
| | CfAmCfUmGfUmU*SmU*SmU | UU | OSS |
| WV-37055 | mU*RfU*SmA*RfU*RmA*SfG*SmA*RfG*RmCfAmAmGmAfAm | UUAUAGAGCAAGAACACUGUU | RSRRSSRROOOOOOOOOOO |
| | CfAmCfUmGfUmU*SmU*SmU | UU | OSS |
| WV-37056 | mU*RfU*SmA*RfU*RmA*SfG*SmA*RfG*SmCfAmAmGmAfAm | UUAUAGAGCAAGAACACUGUU | RSRRSSRSOOOOOOOOOOO |
| | CfAmCfUmGfUmU*SmU*SmU | UU | OSS |
| WV-37057 | mU*RfU*SmA*RfU*RmA*SfG*SmA*SfG*RmCfAmAmGmAfAm | UUAUAGAGCAAGAACACUGUU | RSRRSSSROOOOOOOOOOO |
| | CfAmCfUmGfUmU*SmU*SmU | UU | OSS |
| WV-37058 | mU*RfU*SmA*RfU*RmA*SfG*SmA*SfG*SmCfAmAmGmAfAm | UUAUAGAGCAAGAACACUGUU | RSRRSSSSOOOOOOOOOOO |
| | CfAmCfUmGfUmU*SmU*SmU | UU | OSS |
| WV-37059 | mU*RfU*SmA*RfU*SmA*RfG*RmA*RfG*RmCfAmAmGmAfAm | UUAUAGAGCAAGAACACUGUU | RSRSRRRROOOOOOOOOOO |
| | CfAmCfUmGfUmU*SmU*SmU | UU | OSS |
| WV-37060 | mU*RfU*SmA*RfU*SmA*RfG*RmA*RfG*SmCfAmAmGmAfAm | UUAUAGAGCAAGAACACUGUU | RSRSRRRSOOOOOOOOOOO |
| | CfAmCfUmGfUmU*SmU*SmU | UU | OSS |
| WV-37061 | mU*RfU*SmA*RfU*SmA*RfG*RmA*SfG*RmCfAmAmGmAfAm | UUAUAGAGCAAGAACACUGUU | RSRSRRSROOOOOOOOOOO |
| | CfAmCfUmGfUmU*SmU*SmU | UU | OSS |
| WV-37062 | mU*RfU*SmA*RfU*SmA*RfG*RmA*SfG*SmCfAmAmGmAfAm | UUAUAGAGCAAGAACACUGUU | RSRSRRSSOOOOOOOOOOO |
| | CfAmCfUmGfUmU*SmU*SmU | UU | OSS |
| WV-37063 | mU*RfU*SmA*RfU*SmA*RfG*SmA*RfG*RmCfAmAmGmAfAm | UUAUAGAGCAAGAACACUGUU | RSRSRSRROOOOOOOOOOO |
| | CfAmCfUmGfUmU*SmU*SmU | UU | OSS |
| WV-37064 | mU*RfU*SmA*RfU*SmA*RfG*SmA*RfG*SmCfAmAmGmAfAm | UUAUAGAGCAAGAACACUGUU | RSRSRSRSOOOOOOOOOOO |
| | CfAmCfUmGfUmU*SmU*SmU | UU | OSS |
| WV-37065 | mU*RfU*SmA*RfU*SmA*RfG*SmA*SfG*RmCfAmAmGmAfAm | UUAUAGAGCAAGAACACUGUU | RSRSRSSROOOOOOOOOOO |
| | CfAmCfUmGfUmU*SmU*SmU | UU | OSS |
| WV-37066 | mU*RfU*SmA*RfU*SmA*RfG*SmA*SfG*SmCfAmAmGmAfAm | UUAUAGAGCAAGAACACUGUU | RSRSRSSSOOOOOOOOOOO |
| | CfAmCfUmGfUmU*SmU*SmU | UU | OSS |
| WV-37067 | mU*RfU*SmA*RfU*SmA*SfG*RmA*RfG*RmCfAmAmGmAfAm | UUAUAGAGCAAGAACACUGUU | RSRSSRRROOOOOOOOOOO |
| | CfAmCfUmGfUmU*SmU*SmU | UU | OSS |
| WV-37068 | mU*RfU*SmA*RfU*SmA*SfG*RmA*RfG*SmCfAmAmGmAfAm | UUAUAGAGCAAGAACACUGUU | RSRSSRRSOOOOOOOOOOO |
| | CfAmCfUmGfUmU*SmU*SmU | UU | OSS |
| WV-37069 | mU*RfU*SmA*RfU*SmA*SfG*RmA*SfG*RmCfAmAmGmAfAm | UUAUAGAGCAAGAACACUGUU | RSRSSRSROOOOOOOOOOO |
| | CfAmCfUmGfUmU*SmU*SmU | UU | OSS |
| WV-37070 | mU*RfU*SmA*RfU*SmA*SfG*RmA*SfG*SmCfAmAmGmAfAm | UUAUAGAGCAAGAACACUGUU | RSRSSRSSOOOOOOOOOOO |
| | CfAmCfUmGfUmU*SmU*SmU | UU | OSS |
| WV-37071 | mU*RfU*SmA*RfU*SmA*SfG*SmA*RfG*RmCfAmAmGmAfAm | UUAUAGAGCAAGAACACUGUU | RSRSSSRROOOOOOOOOOO |
| | CfAmCfUmGfUmU*SmU*SmU | UU | OSS |
| WV-37072 | mU*RfU*SmA*RfU*SmA*SfG*SmA*RfG*SmCfAmAmGmAfAm | UUAUAGAGCAAGAACACUGUU | RSRSSSRSOOOOOOOOOOO |
| | CfAmCfUmGfUmU*SmU*SmU | UU | OSS |
| WV-37073 | mU*RfU*SmA*RfU*SmA*SfG*SmA*SfG*RmCfAmAmGmAfAm | UUAUAGAGCAAGAACACUGUU | RSRSSSSROOOOOOOOOOO |
| | CfAmCfUmGfUmU*SmU*SmU | UU | OSS |
| WV-37074 | mU*RfU*SmA*RfU*SmA*SfG*SmA*SfG*SmCfAmAmGmAfAm | UUAUAGAGCAAGAACACUGUU | RSRSSSSSOOOOOOOOOOO |
| | CfAmCfUmGfUmU*SmU*SmU | UU | OSS |
| WV-37075 | mU*RfU*SmA*SfU*RmA*RfG*RmA*RfG*RmCfAmAmGmAfAm | UUAUAGAGCAAGAACACUGUU | RSSRRRRROOOOOOOOOOO |
| | CfAmCfUmGfUmU*SmU*SmU | UU | OSS |
| WV-37076 | mU*RfU*SmA*SfU*RmA*RfG*RmA*RfG*SmCfAmAmGmAfAm | UUAUAGAGCAAGAACACUGUU | RSSRRRRSOOOOOOOOOOO |
| | CfAmCfUmGfUmU*SmU*SmU | UU | OSS |
| WV-37077 | mU*RfU*SmA*SfU*RmA*RfG*RmA*SfG*RmCfAmAmGmAfAm | UUAUAGAGCAAGAACACUGUU | RSSRRRSROOOOOOOOOOO |
| | CfAmCfUmGfUmU*SmU*SmU | UU | OSS |
| WV-37078 | mU*RfU*SmA*SfU*RmA*RfG*RmA*SfG*SmCfAmAmGmAfAm | UUAUAGAGCAAGAACACUGUU | RSSRRRSSOOOOOOOOOOO |
| | CfAmCfUmGfUmU*SmU*SmU | UU | OSS |
| WV-37079 | mU*RfU*SmA*SfU*RmA*RfG*SmA*RfG*RmCfAmAmGmAfAm | UUAUAGAGCAAGAACACUGUU | RSSRRSRROOOOOOOOOOO |
| | CfAmCfUmGfUmU*SmU*SmU | UU | OSS |
| WV-37080 | mU*RfU*SmA*SfU*RmA*RfG*SmA*RfG*SmCfAmAmGmAfAm | UUAUAGAGCAAGAACACUGUU | RSSRRSRSOOOOOOOOOOO |
| | CfAmCfUmGfUmU*SmU*SmU | UU | OSS |
| WV-37081 | mU*RfU*SmA*SfU*RmA*RfG*SmA*SfG*RmCfAmAmGmAfAm | UUAUAGAGCAAGAACACUGUU | RSSRRSSROOOOOOOOOOO |
| | CfAmCfUmGfUmU*SmU*SmU | UU | OSS |
| WV-37082 | mU*RfU*SmA*SfU*RmA*RfG*SmA*SfG*SmCfAmAmGmAfAm | UUAUAGAGCAAGAACACUGUU | RSSRRSSSOOOOOOOOOOO |
| | CfAmCfUmGfUmU*SmU*SmU | UU | OSS |
| WV-37083 | mU*RfU*SmA*SfU*RmA*SfG*RmA*RfG*RmCfAmAmGmAfAm | UUAUAGAGCAAGAACACUGUU | RSSRSRRROOOOOOOOOOO |
| | CfAmCfUmGfUmU*SmU*SmU | UU | OSS |
| WV-37084 | mU*RfU*SmA*SfU*RmA*SfG*RmA*RfG*SmCfAmAmGmAfAm | UUAUAGAGCAAGAACACUGUU | RSSRSRRSOOOOOOOOOOO |
| | CfAmCfUmGfUmU*SmU*SmU | UU | OSS |
| WV-37085 | mU*RfU*SmA*SfU*RmA*SfG*RmA*SfG*RmCfAmAmGmAfAm | UUAUAGAGCAAGAACACUGUU | RSSRSRSROOOOOOOOOOO |
| | CfAmCfUmGfUmU*SmU*SmU | UU | OSS |
| WV-37086 | mU*RfU*SmA*SfU*RmA*SfG*RmA*SfG*SmCfAmAmGmAfAm | UUAUAGAGCAAGAACACUGUU | RSSRSRSSOOOOOOOOOOO |
| | CfAmCfUmGfUmU*SmU*SmU | UU | OSS |
| WV-37087 | mU*RfU*SmA*SfU*RmA*SfG*SmA*RfG*RmCfAmAmGmAfAm | UUAUAGAGCAAGAACACUGUU | RSSRSSRROOOOOOOOOOO |
| | CfAmCfUmGfUmU*SmU*SmU | UU | OSS |
| WV-37088 | mU*RfU*SmA*SfU*RmA*SfG*SmA*RfG*SmCfAmAmGmAfAm | UUAUAGAGCAAGAACACUGUU | RSSRSSRSOOOOOOOOOOO |
| | CfAmCfUmGfUmU*SmU*SmU | UU | OSS |
| WV-37089 | mU*RfU*SmA*SfU*RmA*SfG*SmA*SfG*RmCfAmAmGmAfAm | UUAUAGAGCAAGAACACUGUU | RSSRSSSROOOOOOOOOOO |
| | CfAmCfUmGfUmU*SmU*SmU | UU | OSS |
| WV-37090 | mU*RfU*SmA*SfU*RmA*SfG*SmA*SfG*SmCfAmAmGmAfAm | UUAUAGAGCAAGAACACUGUU | RSSRSSSSOOOOOOOOOOO |
| | CfAmCfUmGfUmU*SmU*SmU | UU | OSS |
| WV-37091 | mU*RfU*SmA*SfU*SmA*RfG*RmA*RfG*RmCfAmAmGmAfAm | UUAUAGAGCAAGAACACUGUU | RSSSRRRROOOOOOOOOOO |
| | CfAmCfUmGfUmU*SmU*SmU | UU | OSS |
| WV-37092 | mU*RfU*SmA*SfU*SmA*RfG*RmA*RfG*SmCfAmAmGmAfAm | UUAUAGAGCAAGAACACUGUU | RSSSRRRSOOOOOOOOOOO |
| | CfAmCfUmGfUmU*SmU*SmU | UU | OSS |
| WV-37093 | mU*RfU*SmA*SfU*SmA*RfG*RmA*SfG*RmCfAmAmGmAfAm | UUAUAGAGCAAGAACACUGUU | RSSSRRSROOOOOOOOOOO |
| | CfAmCfUmGfUmU*SmU*SmU | UU | OSS |
| WV-37094 | mU*RfU*SmA*SfU*SmA*RfG*RmA*SfG*SmCfAmAmGmAfAm | UUAUAGAGCAAGAACACUGUU | RSSSRRSSOOOOOOOOOOO |
| | CfAmCfUmGfUmU*SmU*SmU | UU | OSS |
| WV-37095 | mU*RfU*SmA*SfU*SmA*RfG*SmA*RfG*RmCfAmAmGmAfAm | UUAUAGAGCAAGAACACUGUU | RSSSRSRROOOOOOOOOOO |
| | CfAmCfUmGfUmU*SmU*SmU | UU | OSS |
| WV-37096 | mU*RfU*SmA*SfU*SmA*RfG*SmA*RfG*SmCfAmAmGmAfAm | UUAUAGAGCAAGAACACUGUU | RSSSRSRSOOOOOOOOOOO |
| | CfAmCfUmGfUmU*SmU*SmU | UU | OSS |
| WV-37097 | mU*RfU*SmA*SfU*SmA*RfG*SmA*SfG*RmCfAmAmGmAfAm | UUAUAGAGCAAGAACACUGUU | RSSSRSSROOOOOOOOOOO |
| | CfAmCfUmGfUmU*SmU*SmU | UU | OSS |
| WV-37098 | mU*RfU*SmA*SfU*SmA*RfG*SmA*SfG*SmCfAmAmGmAfAm | UUAUAGAGCAAGAACACUGUU | RSSSRSSSOOOOOOOOOOO |
| | CfAmCfUmGfUmU*SmU*SmU | UU | OSS |
| WV-37099 | mU*RfU*SmA*SfU*SmA*SfG*RmA*RfG*RmCfAmAmGmAfAm | UUAUAGAGCAAGAACACUGUU | RSSSSRRROOOOOOOOOOO |
| | CfAmCfUmGfUmU*SmU*SmU | UU | OSS |
| WV-37100 | mU*RfU*SmA*SfU*SmA*SfG*RmA*RfG*SmCfAmAmGmAfAm | UUAUAGAGCAAGAACACUGUU | RSSSSRRSOOOOOOOOOOO |
| | CfAmCfUmGfUmU*SmU*SmU | UU | OSS |
| WV-37101 | mU*RfU*SmA*SfU*SmA*SfG*RmA*SfG*RmCfAmAmGmAfAm | UUAUAGAGCAAGAACACUGUU | RSSSSRSROOOOOOOOOOO |
| | CfAmCfUmGfUmU*SmU*SmU | UU | OSS |
| WV-37102 | mU*RfU*SmA*SfU*SmA*SfG*RmA*SfG*SmCfAmAmGmAfAm | UUAUAGAGCAAGAACACUGUU | RSSSSRSSOOOOOOOOOOO |
| | CfAmCfUmGfUmU*SmU*SmU | UU | OSS |
| WV-37103 | mU*RfU*SmA*SfU*SmA*SfG*SmA*RfG*RmCfAmAmGmAfAm | UUAUAGAGCAAGAACACUGUU | RSSSSSRROOOOOOOOOOO |
| | CfAmCfUmGfUmU*SmU*SmU | UU | OSS |
| WV-37104 | mU*RfU*SmA*SfU*SmA*SfG*SmA*RfG*SmCfAmAmGmAfAm | UUAUAGAGCAAGAACACUGUU | RSSSSSRSOOOOOOOOOOO |
| | CfAmCfUmGfUmU*SmU*SmU | UU | OSS |
| WV-37105 | mU*RfU*SmA*SfU*SmA*SfG*SmA*SfG*RmCfAmAmGmAfAm | UUAUAGAGCAAGAACACUGUU | RSSSSSSROOOOOOOOOOO |
| | CfAmCfUmGfUmU*SmU*SmU | UU | OSS |
| WV-37106 | mU*RfU*SmA*SfU*SmA*SfG*SmA*SfG*SmCfAmAmGmAfAm | UUAUAGAGCAAGAACACUGUU | RSSSSSSSOOOOOOOOOOO |
| | CfAmCfUmGfUmU*SmU*SmU | UU | OSS |
| WV-37107 | mU*SfU*RmA*RfU*RmA*RfG*RmA*RfG*RmCfAmAmGmAfAm | UUAUAGAGCAAGAACACUGUU | SRRRRRRROOOOOOOOOOO |
| | CfAmCfUmGfUmU*SmU*SmU | UU | OSS |
| WV-37108 | mU*SfU*RmA*RfU*RmA*RfG*RmA*RfG*SmCfAmAmGmAfAm | UUAUAGAGCAAGAACACUGUU | SRRRRRRSOOOOOOOOOOO |
| | CfAmCfUmGfUmU*SmU*SmU | UU | OSS |
| WV-37109 | mU*SfU*RmA*RfU*RmA*RfG*RmA*SfG*RmCfAmAmGmAfAm | UUAUAGAGCAAGAACACUGUU | SRRRRRSROOOOOOOOOOO |
| | CfAmCfUmGfUmU*SmU*SmU | UU | OSS |
| WV-37110 | mU*SfU*RmA*RfU*RmA*RfG*RmA*SfG*SmCfAmAmGmAfAm | UUAUAGAGCAAGAACACUGUU | SRRRRRSSOOOOOOOOOOO |
| | CfAmCfUmGfUmU*SmU*SmU | UU | OSS |
| WV-37111 | mU*SfU*RmA*RfU*RmA*RfG*SmA*RfG*RmCfAmAmGmAfAm | UUAUAGAGCAAGAACACUGUU | SRRRRSRROOOOOOOOOOO |
| | CfAmCfUmGfUmU*SmU*SmU | UU | OSS |
| WV-37112 | mU*SfU*RmA*RfU*RmA*RfG*SmA*RfG*SmCfAmAmGmAfAm | UUAUAGAGCAAGAACACUGUU | SRRRRSRSOOOOOOOOOOO |
| | CfAmCfUmGfUmU*SmU*SmU | UU | OSS |
| WV-37113 | mU*SfU*RmA*RfU*RmA*RfG*SmA*SfG*RmCfAmAmGmAfAm | UUAUAGAGCAAGAACACUGUU | SRRRRSSROOOOOOOOOOO |
| | CfAmCfUmGfUmU*SmU*SmU | UU | OSS |
| WV-37114 | mU*SfU*RmA*RfU*RmA*RfG*SmA*SfG*SmCfAmAmGmAfAm | UUAUAGAGCAAGAACACUGUU | SRRRRSSSOOOOOOOOOOO |
| | CfAmCfUmGfUmU*SmU*SmU | UU | OSS |
| WV-37115 | mU*SfU*RmA*RfU*RmA*SfG*RmA*RfG*RmCfAmAmGmAfAm | UUAUAGAGCAAGAACACUGUU | SRRRSRRROOOOOOOOOOO |
| | CfAmCfUmGfUmU*SmU*SmU | UU | OSS |
| WV-37116 | mU*SfU*RmA*RfU*RmA*SfG*RmA*RfG*SmCfAmAmGmAfAm | UUAUAGAGCAAGAACACUGUU | SRRRSRRSOOOOOOOOOOO |
| | CfAmCfUmGfUmU*SmU*SmU | UU | OSS |
| WV-37117 | mU*SfU*RmA*RfU*RmA*SfG*RmA*SfG*RmCfAmAmGmAfAm | UUAUAGAGCAAGAACACUGUU | SRRRSRSROOOOOOOOOOO |
| | CfAmCfUmGfUmU*SmU*SmU | UU | OSS |
| WV-37118 | mU*SfU*RmA*RfU*RmA*SfG*RmA*SfG*SmCfAmAmGmAfAm | UUAUAGAGCAAGAACACUGUU | SRRRSRSSOOOOOOOOOOO |
| | CfAmCfUmGfUmU*SmU*SmU | UU | OSS |
| WV-37119 | mU*SfU*RmA*RfU*RmA*SfG*SmA*RfG*RmCfAmAmGmAfAm | UUAUAGAGCAAGAACACUGUU | SRRRSSRROOOOOOOOOOO |
| | CfAmCfUmGfUmU*SmU*SmU | UU | OSS |
| WV-37120 | mU*SfU*RmA*RfU*RmA*SfG*SmA*RfG*SmCfAmAmGmAfAm | UUAUAGAGCAAGAACACUGUU | SRRRSSRSOOOOOOOOOOO |
| | CfAmCfUmGfUmU*SmU*SmU | UU | OSS |
| WV-37121 | mU*SfU*RmA*RfU*RmA*SfG*SmA*SfG*RmCfAmAmGmAfAm | UUAUAGAGCAAGAACACUGUU | SRRRSSSROOOOOOOOOOO |
| | CfAmCfUmGfUmU*SmU*SmU | UU | OSS |
| WV-37122 | mU*SfU*RmA*RfU*RmA*SfG*SmA*SfG*SmCfAmAmGmAfAm | UUAUAGAGCAAGAACACUGUU | SRRRSSSSOOOOOOOOOOO |
| | CfAmCfUmGfUmU*SmU*SmU | UU | OSS |
| WV-37123 | mU*SfU*RmA*RfU*SmA*RfG*RmA*RfG*RmCfAmAmGmAfAm | UUAUAGAGCAAGAACACUGUU | SRRSRRRROOOOOOOOOOO |
| | CfAmCfUmGfUmU*SmU*SmU | UU | OSS |
| WV-37124 | mU*SfU*RmA*RfU*SmA*RfG*RmA*RfG*SmCfAmAmGmAfAm | UUAUAGAGCAAGAACACUGUU | SRRSRRRSOOOOOOOOOOO |
| | CfAmCfUmGfUmU*SmU*SmU | UU | OSS |
| WV-37125 | mU*SfU*RmA*RfU*SmA*RfG*RmA*SfG*RmCfAmAmGmAfAm | UUAUAGAGCAAGAACACUGUU | SRRSRRSROOOOOOOOOOO |
| | CfAmCfUmGfUmU*SmU*SmU | UU | OSS |
| WV-37126 | mU*SfU*RmA*RfU*SmA*RfG*RmA*SfG*SmCfAmAmGmAfAm | UUAUAGAGCAAGAACACUGUU | SRRSRRSSOOOOOOOOOOO |
| | CfAmCfUmGfUmU*SmU*SmU | UU | OSS |
| WV-37127 | mU*SfU*RmA*RfU*SmA*RfG*SmA*RfG*RmCfAmAmGmAfAm | UUAUAGAGCAAGAACACUGUU | SRRSRSRROOOOOOOOOOO |
| | CfAmCfUmGfUmU*SmU*SmU | UU | OSS |
| WV-37128 | mU*SfU*RmA*RfU*SmA*RfG*SmA*RfG*SmCfAmAmGmAfAm | UUAUAGAGCAAGAACACUGUU | SRRSRSRSOOOOOOOOOOO |
| | CfAmCfUmGfUmU*SmU*SmU | UU | OSS |
| WV-37129 | mU*SfU*RmA*RfU*SmA*RfG*SmA*SfG*RmCfAmAmGmAfAm | UUAUAGAGCAAGAACACUGUU | SRRSRSSROOOOOOOOOOO |
| | CfAmCfUmGfUmU*SmU*SmU | UU | OSS |
| WV-37130 | mU*SfU*RmA*RfU*SmA*RfG*SmA*SfG*SmCfAmAmGmAfAm | UUAUAGAGCAAGAACACUGUU | SRRSRSSSOOOOOOOOOOO |
| | CfAmCfUmGfUmU*SmU*SmU | UU | OSS |
| WV-37131 | mU*SfU*RmA*RfU*SmA*SfG*RmA*RfG*RmCfAmAmGmAfAm | UUAUAGAGCAAGAACACUGUU | SRRSSRRROOOOOOOOOOO |
| | CfAmCfUmGfUmU*SmU*SmU | UU | OSS |
| WV-37132 | mU*SfU*RmA*RfU*SmA*SfG*RmA*RfG*SmCfAmAmGmAfAm | UUAUAGAGCAAGAACACUGUU | SRRSSRRSOOOOOOOOOOO |
| | CfAmCfUmGfUmU*SmU*SmU | UU | OSS |
| WV-37133 | mU*SfU*RmA*RfU*SmA*SfG*RmA*SfG*RmCfAmAmGmAfAm | UUAUAGAGCAAGAACACUGUU | SRRSSRSROOOOOOOOOOO |
| | CfAmCfUmGfUmU*SmU*SmU | UU | OSS |
| WV-37134 | mU*SfU*RmA*RfU*SmA*SfG*RmA*SfG*SmCfAmAmGmAfAm | UUAUAGAGCAAGAACACUGUU | SRRSSRSSOOOOOOOOOOO |
| | CfAmCfUmGfUmU*SmU*SmU | UU | OSS |
| WV-37135 | mU*SfU*RmA*RfU*SmA*SfG*SmA*RfG*RmCfAmAmGmAfAm | UUAUAGAGCAAGAACACUGUU | SRRSSSRROOOOOOOOOOO |
| | CfAmCfUmGfUmU*SmU*SmU | UU | OSS |
| WV-37136 | mU*SfU*RmA*RfU*SmA*SfG*SmA*RfG*SmCfAmAmGmAfAm | UUAUAGAGCAAGAACACUGUU | SRRSSSRSOOOOOOOOOOO |
| | CfAmCfUmGfUmU*SmU*SmU | UU | OSS |
| WV-37137 | mU*SfU*RmA*RfU*SmA*SfG*SmA*SfG*RmCfAmAmGmAfAm | UUAUAGAGCAAGAACACUGUU | SRRSSSSROOOOOOOOOOO |
| | CfAmCfUmGfUmU*SmU*SmU | UU | OSS |
| WV-37138 | mU*SfU*RmA*RfU*SmA*SfG*SmA*SfG*SmCfAmAmGmAfAm | UUAUAGAGCAAGAACACUGUU | SRRSSSSSOOOOOOOOOOO |
| | CfAmCfUmGfUmU*SmU*SmU | UU | OSS |
| WV-37139 | mU*SfU*RmA*SfU*RmA*RfG*RmA*RfG*RmCfAmAmGmAfAm | UUAUAGAGCAAGAACACUGUU | SRSRRRRROOOOOOOOOOO |
| | CfAmCfUmGfUmU*SmU*SmU | UU | OSS |
| WV-37140 | mU*SfU*RmA*SfU*RmA*RfG*RmA*RfG*SmCfAmAmGmAfAm | UUAUAGAGCAAGAACACUGUU | SRSRRRRSOOOOOOOOOOO |
| | CfAmCfUmGfUmU*SmU*SmU | UU | OSS |
| WV-37141 | mU*SfU*RmA*SfU*RmA*RfG*RmA*SfG*RmCfAmAmGmAfAm | UUAUAGAGCAAGAACACUGUU | SRSRRRSROOOOOOOOOOO |
| | CfAmCfUmGfUmU*SmU*SmU | UU | OSS |
| WV-37142 | mU*SfU*RmA*SfU*RmA*RfG*RmA*SfG*SmCfAmAmGmAfAm | UUAUAGAGCAAGAACACUGUU | SRSRRRSSOOOOOOOOOOO |
| | CfAmCfUmGfUmU*SmU*SmU | UU | OSS |
| WV-37143 | mU*SfU*RmA*SfU*RmA*RfG*SmA*RfG*RmCfAmAmGmAfAm | UUAUAGAGCAAGAACACUGUU | SRSRRSRROOOOOOOOOOO |
| | CfAmCfUmGfUmU*SmU*SmU | UU | OSS |
| WV-37144 | mU*SfU*RmA*SfU*RmA*RfG*SmA*RfG*SmCfAmAmGmAfAm | UUAUAGAGCAAGAACACUGUU | SRSRRSRSOOOOOOOOOOO |
| | CfAmCfUmGfUmU*SmU*SmU | UU | OSS |
| WV-37145 | mU*SfU*RmA*SfU*RmA*RfG*SmA*SfG*RmCfAmAmGmAfAm | UUAUAGAGCAAGAACACUGUU | SRSRRSSROOOOOOOOOOO |
| | CfAmCfUmGfUmU*SmU*SmU | UU | OSS |
| WV-37146 | mU*SfU*RmA*SfU*RmA*RfG*SmA*SfG*SmCfAmAmGmAfAm | UUAUAGAGCAAGAACACUGUU | SRSRRSSSOOOOOOOOOOO |
| | CfAmCfUmGfUmU*SmU*SmU | UU | OSS |
| WV-37147 | mU*SfU*RmA*SfU*RmA*SfG*RmA*RfG*RmCfAmAmGmAfAm | UUAUAGAGCAAGAACACUGUU | SRSRSRRROOOOOOOOOOO |
| | CfAmCfUmGfUmU*SmU*SmU | UU | OSS |
| WV-37148 | mU*SfU*RmA*SfU*RmA*SfG*RmA*RfG*SmCfAmAmGmAfAm | UUAUAGAGCAAGAACACUGUU | SRSRSRRSOOOOOOOOOOO |
| | CfAmCfUmGfUmU*SmU*SmU | UU | OSS |
| WV-37149 | mU*SfU*RmA*SfU*RmA*SfG*RmA*SfG*RmCfAmAmGmAfAm | UUAUAGAGCAAGAACACUGUU | SRSRSRSROOOOOOOOOOO |
| | CfAmCfUmGfUmU*SmU*SmU | UU | OSS |
| WV-37150 | mU*SfU*RmA*SfU*RmA*SfG*RmA*SfG*SmCfAmAmGmAfAm | UUAUAGAGCAAGAACACUGUU | SRSRSRSSOOOOOOOOOOO |
| | CfAmCfUmGfUmU*SmU*SmU | UU | OSS |
| WV-37151 | mU*SfU*RmA*SfU*RmA*SfG*SmA*RfG*RmCfAmAmGmAfAm | UUAUAGAGCAAGAACACUGUU | SRSRSSRROOOOOOOOOOO |
| | CfAmCfUmGfUmU*SmU*SmU | UU | OSS |
| WV-37152 | mU*SfU*RmA*SfU*RmA*SfG*SmA*RfG*SmCfAmAmGmAfAm | UUAUAGAGCAAGAACACUGUU | SRSRSSRSOOOOOOOOOOO |
| | CfAmCfUmGfUmU*SmU*SmU | UU | OSS |
| WV-37153 | mU*SfU*RmA*SfU*RmA*SfG*SmA*SfG*RmCfAmAmGmAfAm | UUAUAGAGCAAGAACACUGUU | SRSRSSSROOOOOOOOOOO |
| | CfAmCfUmGfUmU*SmU*SmU | UU | OSS |
| WV-37154 | mU*SfU*RmA*SfU*RmA*SfG*SmA*SfG*SmCfAmAmGmAfAm | UUAUAGAGCAAGAACACUGUU | SRSRSSSSOOOOOOOOOOO |
| | CfAmCfUmGfUmU*SmU*SmU | UU | OSS |
| WV-37155 | mU*SfU*RmA*SfU*SmA*RfG*RmA*RfG*RmCfAmAmGmAfAm | UUAUAGAGCAAGAACACUGUU | SRSSRRRROOOOOOOOOOO |
| | CfAmCfUmGfUmU*SmU*SmU | UU | OSS |
| WV-37156 | mU*SfU*RmA*SfU*SmA*RfG*RmA*RfG*SmCfAmAmGmAfAm | UUAUAGAGCAAGAACACUGUU | SRSSRRRSOOOOOOOOOOO |
| | CfAmCfUmGfUmU*SmU*SmU | UU | OSS |
| WV-37157 | mU*SfU*RmA*SfU*SmA*RfG*RmA*SfG*RmCfAmAmGmAfAm | UUAUAGAGCAAGAACACUGUU | SRSSRRSROOOOOOOOOOO |
| | CfAmCfUmGfUmU*SmU*SmU | UU | OSS |
| WV-37158 | mU*SfU*RmA*SfU*SmA*RfG*RmA*SfG*SmCfAmAmGmAfAm | UUAUAGAGCAAGAACACUGUU | SRSSRRSSOOOOOOOOOOO |
| | CfAmCfUmGfUmU*SmU*SmU | UU | OSS |
| WV-37159 | mU*SfU*RmA*SfU*SmA*RfG*SmA*RfG*RmCfAmAmGmAfAm | UUAUAGAGCAAGAACACUGUU | SRSSRSRROOOOOOOOOOO |
| | CfAmCfUmGfUmU*SmU*SmU | UU | OSS |
| WV-37160 | mU*SfU*RmA*SfU*SmA*RfG*SmA*RfG*SmCfAmAmGmAfAm | UUAUAGAGCAAGAACACUGUU | SRSSRSRSOOOOOOOOOOO |
| | CfAmCfUmGfUmU*SmU*SmU | UU | OSS |
| WV-37161 | mU*SfU*RmA*SfU*SmA*RfG*SmA*SfG*RmCfAmAmGmAfAm | UUAUAGAGCAAGAACACUGUU | SRSSRSSROOOOOOOOOOO |
| | CfAmCfUmGfUmU*SmU*SmU | UU | OSS |
| WV-37162 | mU*SfU*RmA*SfU*SmA*RfG*SmA*SfG*SmCfAmAmGmAfAm | UUAUAGAGCAAGAACACUGUU | SRSSRSSSOOOOOOOOOOO |
| | CfAmCfUmGfUmU*SmU*SmU | UU | OSS |
| WV-37163 | mU*SfU*RmA*SfU*SmA*SfG*RmA*RfG*RmCfAmAmGmAfAm | UUAUAGAGCAAGAACACUGUU | SRSSSRRROOOOOOOOOOO |
| | CfAmCfUmGfUmU*SmU*SmU | UU | OSS |
| WV-37164 | mU*SfU*RmA*SfU*SmA*SfG*RmA*RfG*SmCfAmAmGmAfAm | UUAUAGAGCAAGAACACUGUU | SRSSSRRSOOOOOOOOOOO |
| | CfAmCfUmGfUmU*SmU*SmU | UU | OSS |
| WV-37165 | mU*SfU*RmA*SfU*SmA*SfG*RmA*SfG*RmCfAmAmGmAfAm | UUAUAGAGCAAGAACACUGUU | SRSSSRSROOOOOOOOOOO |
| | CfAmCfUmGfUmU*SmU*SmU | UU | OSS |
| WV-37166 | mU*SfU*RmA*SfU*SmA*SfG*RmA*SfG*SmCfAmAmGmAfAm | UUAUAGAGCAAGAACACUGUU | SRSSSRSSOOOOOOOOOOO |
| | CfAmCfUmGfUmU*SmU*SmU | UU | OSS |
| WV-37167 | mU*SfU*RmA*SfU*SmA*SfG*SmA*RfG*RmCfAmAmGmAfAm | UUAUAGAGCAAGAACACUGUU | SRSSSSRROOOOOOOOOOO |
| | CfAmCfUmGfUmU*SmU*SmU | UU | OSS |
| WV-37168 | mU*SfU*RmA*SfU*SmA*SfG*SmA*RfG*SmCfAmAmGmAfAm | UUAUAGAGCAAGAACACUGUU | SRSSSSRSOOOOOOOOOOO |
| | CfAmCfUmGfUmU*SmU*SmU | UU | OSS |
| WV-37169 | mU*SfU*RmA*SfU*SmA*SfG*SmA*SfG*RmCfAmAmGmAfAm | UUAUAGAGCAAGAACACUGUU | SRSSSSSROOOOOOOOOOO |
| | CfAmCfUmGfUmU*SmU*SmU | UU | OSS |
| WV-37170 | mU*SfU*RmA*SfU*SmA*SfG*SmA*SfG*SmCfAmAmGmAfAm | UUAUAGAGCAAGAACACUGUU | SRSSSSSSOOOOOOOOOOO |
| | CfAmCfUmGfUmU*SmU*SmU | UU | OSS |
| WV-37171 | mU*SfU*SmA*RfU*RmA*RfG*RmA*RfG*RmCfAmAmGmAfAm | UUAUAGAGCAAGAACACUGUU | SSRRRRRROOOOOOOOOOO |
| | CfAmCfUmGfUmU*SmU*SmU | UU | OSS |
| WV-37172 | mU*SfU*SmA*RfU*RmA*RfG*RmA*RfG*SmCfAmAmGmAfAm | UUAUAGAGCAAGAACACUGUU | SSRRRRRSOOOOOOOOOOO |
| | CfAmCfUmGfUmU*SmU*SmU | UU | OSS |
| WV-37173 | mU*SfU*SmA*RfU*RmA*RfG*RmA*SfG*RmCfAmAmGmAfAm | UUAUAGAGCAAGAACACUGUU | SSRRRRSROOOOOOOOOOO |
| | CfAmCfUmGfUmU*SmU*SmU | UU | OSS |
| WV-37174 | mU*SfU*SmA*RfU*RmA*RfG*RmA*SfG*SmCfAmAmGmAfAm | UUAUAGAGCAAGAACACUGUU | SSRRRRSSOOOOOOOOOOO |
| | CfAmCfUmGfUmU*SmU*SmU | UU | OSS |
| WV-37175 | mU*SfU*SmA*RfU*RmA*RfG*SmA*RfG*RmCfAmAmGmAfAm | UUAUAGAGCAAGAACACUGUU | SSRRRSRROOOOOOOOOOO |
| | CfAmCfUmGfUmU*SmU*SmU | UU | OSS |
| WV-37176 | mU*SfU*SmA*RfU*RmA*RfG*SmA*RfG*SmCfAmAmGmAfAm | UUAUAGAGCAAGAACACUGUU | SSRRRSRSOOOOOOOOOOO |
| | CfAmCfUmGfUmU*SmU*SmU | UU | OSS |
| WV-37177 | mU*SfU*SmA*RfU*RmA*RfG*SmA*SfG*RmCfAmAmGmAfAm | UUAUAGAGCAAGAACACUGUU | SSRRRSSROOOOOOOOOOO |
| | CfAmCfUmGfUmU*SmU*SmU | UU | OSS |
| WV-37178 | mU*SfU*SmA*RfU*RmA*RfG*SmA*SfG*SmCfAmAmGmAfAm | UUAUAGAGCAAGAACACUGUU | SSRRRSSSOOOOOOOOOOO |
| | CfAmCfUmGfUmU*SmU*SmU | UU | OSS |
| WV-37179 | mU*SfU*SmA*RfU*RmA*SfG*RmA*RfG*RmCfAmAmGmAfAm | UUAUAGAGCAAGAACACUGUU | SSRRSRRROOOOOOOOOOO |
| | CfAmCfUmGfUmU*SmU*SmU | UU | OSS |
| WV-37180 | mU*SfU*SmA*RfU*RmA*SfG*RmA*RfG*SmCfAmAmGmAfAm | UUAUAGAGCAAGAACACUGUU | SSRRSRRSOOOOOOOOOOO |
| | CfAmCfUmGfUmU*SmU*SmU | UU | OSS |
| WV-37181 | mU*SfU*SmA*RfU*RmA*SfG*RmA*SfG*RmCfAmAmGmAfAm | UUAUAGAGCAAGAACACUGUU | SSRRSRSROOOOOOOOOOO |
| | CfAmCfUmGfUmU*SmU*SmU | UU | OSS |
| WV-37182 | mU*SfU*SmA*RfU*RmA*SfG*RmA*SfG*SmCfAmAmGmAfAm | UUAUAGAGCAAGAACACUGUU | SSRRSRSSOOOOOOOOOOO |
| | CfAmCfUmGfUmU*SmU*SmU | UU | OSS |
| WV-37183 | mU*SfU*SmA*RfU*RmA*SfG*SmA*RfG*RmCfAmAmGmAfAm | UUAUAGAGCAAGAACACUGUU | SSRRSSRROOOOOOOOOOO |
| | CfAmCfUmGfUmU*SmU*SmU | UU | OSS |
| WV-37184 | mU*SfU*SmA*RfU*RmA*SfG*SmA*RfG*SmCfAmAmGmAfAm | UUAUAGAGCAAGAACACUGUU | SSRRSSRSOOOOOOOOOOO |
| | CfAmCfUmGfUmU*SmU*SmU | UU | OSS |
| WV-37185 | mU*SfU*SmA*RfU*RmA*SfG*SmA*SfG*RmCfAmAmGmAfAm | UUAUAGAGCAAGAACACUGUU | SSRRSSSROOOOOOOOOOO |
| | CfAmCfUmGfUmU*SmU*SmU | UU | OSS |
| WV-37186 | mU*SfU*SmA*RfU*RmA*SfG*SmA*SfG*SmCfAmAmGmAfAm | UUAUAGAGCAAGAACACUGUU | SSRRSSSSOOOOOOOOOOO |
| | CfAmCfUmGfUmU*SmU*SmU | UU | OSS |
| WV-37187 | mU*SfU*SmA*RfU*SmA*RfG*RmA*RfG*RmCfAmAmGmAfAm | UUAUAGAGCAAGAACACUGUU | SSRSRRRROOOOOOOOOOO |
| | CfAmCfUmGfUmU*SmU*SmU | UU | OSS |
| WV-37188 | mU*SfU*SmA*RfU*SmA*RfG*RmA*RfG*SmCfAmAmGmAfAm | UUAUAGAGCAAGAACACUGUU | SSRSRRRSOOOOOOOOOOO |
| | CfAmCfUmGfUmU*SmU*SmU | UU | OSS |
| WV-37189 | mU*SfU*SmA*RfU*SmA*RfG*RmA*SfG*RmCfAmAmGmAfAm | UUAUAGAGCAAGAACACUGUU | SSRSRRSROOOOOOOOOOO |
| | CfAmCfUmGfUmU*SmU*SmU | UU | OSS |
| WV-37190 | mU*SfU*SmA*RfU*SmA*RfG*RmA*SfG*SmCfAmAmGmAfAm | UUAUAGAGCAAGAACACUGUU | SSRSRRSSOOOOOOOOOOO |
| | CfAmCfUmGfUmU*SmU*SmU | UU | OSS |
| WV-37191 | mU*SfU*SmA*RfU*SmA*RfG*SmA*RfG*RmCfAmAmGmAfAm | UUAUAGAGCAAGAACACUGUU | SSRSRSRROOOOOOOOOOO |
| | CfAmCfUmGfUmU*SmU*SmU | UU | OSS |
| WV-37192 | mU*SfU*SmA*RfU*SmA*RfG*SmA*RfG*SmCfAmAmGmAfAm | UUAUAGAGCAAGAACACUGUU | SSRSRSRSOOOOOOOOOOO |
| | CfAmCfUmGfUmU*SmU*SmU | UU | OSS |
| WV-37193 | mU*SfU*SmA*RfU*SmA*RfG*SmA*SfG*RmCfAmAmGmAfAm | UUAUAGAGCAAGAACACUGUU | SSRSRSSROOOOOOOOOOO |
| | CfAmCfUmGfUmU*SmU*SmU | UU | OSS |
| WV-37194 | mU*SfU*SmA*RfU*SmA*RfG*SmA*SfG*SmCfAmAmGmAfAm | UUAUAGAGCAAGAACACUGUU | SSRSRSSSOOOOOOOOOOO |
| | CfAmCfUmGfUmU*SmU*SmU | UU | OSS |
| WV-37195 | mU*SfU*SmA*RfU*SmA*SfG*RmA*RfG*RmCfAmAmGmAfAm | UUAUAGAGCAAGAACACUGUU | SSRSSRRROOOOOOOOOOO |
| | CfAmCfUmGfUmU*SmU*SmU | UU | OSS |
| WV-37196 | mU*SfU*SmA*RfU*SmA*SfG*RmA*RfG*SmCfAmAmGmAfAm | UUAUAGAGCAAGAACACUGUU | SSRSSRRSOOOOOOOOOOO |
| | CfAmCfUmGfUmU*SmU*SmU | UU | OSS |
| WV-37197 | mU*SfU*SmA*RfU*SmA*SfG*RmA*SfG*RmCfAmAmGmAfAm | UUAUAGAGCAAGAACACUGUU | SSRSSRSROOOOOOOOOOO |
| | CfAmCfUmGfUmU*SmU*SmU | UU | OSS |
| WV-37198 | mU*SfU*SmA*RfU*SmA*SfG*RmA*SfG*SmCfAmAmGmAfAm | UUAUAGAGCAAGAACACUGUU | SSRSSRSSOOOOOOOOOOO |
| | CfAmCfUmGfUmU*SmU*SmU | UU | OSS |
| WV-37199 | mU*SfU*SmA*RfU*SmA*SfG*SmA*RfG*RmCfAmAmGmAfAm | UUAUAGAGCAAGAACACUGUU | SSRSSSRROOOOOOOOOOO |
| | CfAmCfUmGfUmU*SmU*SmU | UU | OSS |
| WV-37200 | mU*SfU*SmA*RfU*SmA*SfG*SmA*RfG*SmCfAmAmGmAfAm | UUAUAGAGCAAGAACACUGUU | SSRSSSRSOOOOOOOOOOO |
| | CfAmCfUmGfUmU*SmU*SmU | UU | OSS |
| WV-37201 | mU*SfU*SmA*RfU*SmA*SfG*SmA*SfG*RmCfAmAmGmAfAm | UUAUAGAGCAAGAACACUGUU | SSRSSSSROOOOOOOOOOO |
| | CfAmCfUmGfUmU*SmU*SmU | UU | OSS |
| WV-37202 | mU*SfU*SmA*RfU*SmA*SfG*SmA*SfG*SmCfAmAmGmAfAm | UUAUAGAGCAAGAACACUGUU | SSRSSSSSOOOOOOOOOOO |
| | CfAmCfUmGfUmU*SmU*SmU | UU | OSS |
| WV-37203 | mU*SfU*SmA*SfU*RmA*RfG*RmA*RfG*RmCfAmAmGmAfAm | UUAUAGAGCAAGAACACUGUU | SSSRRRRROOOOOOOOOOO |
| | CfAmCfUmGfUmU*SmU*SmU | UU | OSS |
| WV-37204 | mU*SfU*SmA*SfU*RmA*RfG*RmA*RfG*SmCfAmAmGmAfAm | UUAUAGAGCAAGAACACUGUU | SSSRRRRSOOOOOOOOOOO |
| | CfAmCfUmGfUmU*SmU*SmU | UU | OSS |
| WV-37205 | mU*SfU*SmA*SfU*RmA*RfG*RmA*SfG*RmCfAmAmGmAfAm | UUAUAGAGCAAGAACACUGUU | SSSRRRSROOOOOOOOOOO |
| | CfAmCfUmGfUmU*SmU*SmU | UU | OSS |
| WV-37206 | mU*SfU*SmA*SfU*RmA*RfG*RmA*SfG*SmCfAmAmGmAfAm | UUAUAGAGCAAGAACACUGUU | SSSRRRSSOOOOOOOOOOO |
| | CfAmCfUmGfUmU*SmU*SmU | UU | OSS |
| WV-37207 | mU*SfU*SmA*SfU*RmA*RfG*SmA*RfG*RmCfAmAmGmAfAm | UUAUAGAGCAAGAACACUGUU | SSSRRSRROOOOOOOOOOO |
| | CfAmCfUmGfUmU*SmU*SmU | UU | OSS |
| WV-37208 | mU*SfU*SmA*SfU*RmA*RfG*SmA*RfG*SmCfAmAmGmAfAm | UUAUAGAGCAAGAACACUGUU | SSSRRSRSOOOOOOOOOOO |
| | CfAmCfUmGfUmU*SmU*SmU | UU | OSS |
| WV-37209 | mU*SfU*SmA*SfU*RmA*RfG*SmA*SfG*RmCfAmAmGmAfAm | UUAUAGAGCAAGAACACUGUU | SSSRRSSROOOOOOOOOOO |
| | CfAmCfUmGfUmU*SmU*SmU | UU | OSS |
| WV-37210 | mU*SfU*SmA*SfU*RmA*RfG*SmA*SfG*SmCfAmAmGmAfAm | UUAUAGAGCAAGAACACUGUU | SSSRRSSSOOOOOOOOOOO |
| | CfAmCfUmGfUmU*SmU*SmU | UU | OSS |
| WV-37211 | mU*SfU*SmA*SfU*RmA*SfG*RmA*RfG*RmCfAmAmGmAfAm | UUAUAGAGCAAGAACACUGUU | SSSRSRRROOOOOOOOOOO |
| | CfAmCfUmGfUmU*SmU*SmU | UU | OSS |
| WV-37212 | mU*SfU*SmA*SfU*RmA*SfG*RmA*RfG*SmCfAmAmGmAfAm | UUAUAGAGCAAGAACACUGUU | SSSRSRRSOOOOOOOOOOO |
| | CfAmCfUmGfUmU*SmU*SmU | UU | OSS |
| WV-37213 | mU*SfU*SmA*SfU*RmA*SfG*RmA*SfG*RmCfAmAmGmAfAm | UUAUAGAGCAAGAACACUGUU | SSSRSRSROOOOOOOOOOO |
| | CfAmCfUmGfUmU*SmU*SmU | UU | OSS |
| WV-37214 | mU*SfU*SmA*SfU*RmA*SfG*RmA*SfG*SmCfAmAmGmAfAm | UUAUAGAGCAAGAACACUGUU | SSSRSRSSOOOOOOOOOOO |
| | CfAmCfUmGfUmU*SmU*SmU | UU | OSS |
| WV-37215 | mU*SfU*SmA*SfU*RmA*SfG*SmA*RfG*RmCfAmAmGmAfAm | UUAUAGAGCAAGAACACUGUU | SSSRSSRROOOOOOOOOOO |
| | CfAmCfUmGfUmU*SmU*SmU | UU | OSS |
| WV-37216 | mU*SfU*SmA*SfU*RmA*SfG*SmA*RfG*SmCfAmAmGmAfAm | UUAUAGAGCAAGAACACUGUU | SSSRSSRSOOOOOOOOOOO |
| | CfAmCfUmGfUmU*SmU*SmU | UU | OSS |
| WV-37217 | mU*SfU*SmA*SfU*RmA*SfG*SmA*SfG*RmCfAmAmGmAfAm | UUAUAGAGCAAGAACACUGUU | SSSRSSSROOOOOOOOOOO |
| | CfAmCfUmGfUmU*SmU*SmU | UU | OSS |
| WV-37218 | mU*SfU*SmA*SfU*RmA*SfG*SmA*SfG*SmCfAmAmGmAfAm | UUAUAGAGCAAGAACACUGUU | SSSRSSSSOOOOOOOOOOO |
| | CfAmCfUmGfUmU*SmU*SmU | UU | OSS |
| WV-37219 | mU*SfU*SmA*SfU*SmA*RfG*RmA*RfG*RmCfAmAmGmAfAm | UUAUAGAGCAAGAACACUGUU | SSSSRRRROOOOOOOOOOO |
| | CfAmCfUmGfUmU*SmU*SmU | UU | OSS |
| WV-37220 | mU*SfU*SmA*SfU*SmA*RfG*RmA*RfG*SmCfAmAmGmAfAm | UUAUAGAGCAAGAACACUGUU | SSSSRRRSOOOOOOOOOOO |
| | CfAmCfUmGfUmU*SmU*SmU | UU | OSS |
| WV-37221 | mU*SfU*SmA*SfU*SmA*RfG*RmA*SfG*RmCfAmAmGmAfAm | UUAUAGAGCAAGAACACUGUU | SSSSRRSROOOOOOOOOOO |
| | CfAmCfUmGfUmU*SmU*SmU | UU | OSS |
| WV-37222 | mU*SfU*SmA*SfU*SmA*RfG*RmA*SfG*SmCfAmAmGmAfAm | UUAUAGAGCAAGAACACUGUU | SSSSRRSSOOOOOOOOOOO |
| | CfAmCfUmGfUmU*SmU*SmU | UU | OSS |
| WV-37223 | mU*SfU*SmA*SfU*SmA*RfG*SmA*RfG*RmCfAmAmGmAfAm | UUAUAGAGCAAGAACACUGUU | SSSSRSRROOOOOOOOOOO |
| | CfAmCfUmGfUmU*SmU*SmU | UU | OSS |
| WV-37224 | mU*SfU*SmA*SfU*SmA*RfG*SmA*RfG*SmCfAmAmGmAfAm | UUAUAGAGCAAGAACACUGUU | SSSSRSRSOOOOOOOOOOO |
| | CfAmCfUmGfUmU*SmU*SmU | UU | OSS |
| WV-37225 | mU*SfU*SmA*SfU*SmA*RfG*SmA*SfG*RmCfAmAmGmAfAm | UUAUAGAGCAAGAACACUGUU | SSSSRSSROOOOOOOOOOO |
| | CfAmCfUmGfUmU*SmU*SmU | UU | OSS |
| WV-37226 | mU*SfU*SmA*SfU*SmA*RfG*SmA*SfG*SmCfAmAmGmAfAm | UUAUAGAGCAAGAACACUGUU | SSSSRSSSOOOOOOOOOOO |
| | CfAmCfUmGfUmU*SmU*SmU | UU | OSS |
| WV-37227 | mU*SfU*SmA*SfU*SmA*SfG*RmA*RfG*RmCfAmAmGmAfAm | UUAUAGAGCAAGAACACUGUU | SSSSSRRROOOOOOOOOOO |
| | CfAmCfUmGfUmU*SmU*SmU | UU | OSS |
| WV-37228 | mU*SfU*SmA*SfU*SmA*SfG*RmA*RfG*SmCfAmAmGmAfAm | UUAUAGAGCAAGAACACUGUU | SSSSSRRSOOOOOOOOOOO |
| | CfAmCfUmGfUmU*SmU*SmU | UU | OSS |
| WV-37229 | mU*SfU*SmA*SfU*SmA*SfG*RmA*SfG*RmCfAmAmGmAfAm | UUAUAGAGCAAGAACACUGUU | SSSSSRSROOOOOOOOOOO |
| | CfAmCfUmGfUmU*SmU*SmU | UU | OSS |
| WV-37230 | mU*SfU*SmA*SfU*SmA*SfG*RmA*SfG*SmCfAmAmGmAfAm | UUAUAGAGCAAGAACACUGUU | SSSSSRSSOOOOOOOOOOO |
| | CfAmCfUmGfUmU*SmU*SmU | UU | OSS |
| WV-37231 | mU*SfU*SmA*SfU*SmA*SfG*SmA*RfG*RmCfAmAmGmAfAm | UUAUAGAGCAAGAACACUGUU | SSSSSSRROOOOOOOOOOO |
| | CfAmCfUmGfUmU*SmU*SmU | UU | OSS |
| WV-37232 | mU*SfU*SmA*SfU*SmA*SfG*SmA*RfG*SmCfAmAmGmAfAm | UUAUAGAGCAAGAACACUGUU | SSSSSSRSOOOOOOOOOOO |
| | CfAmCfUmGfUmU*SmU*SmU | UU | OSS |
| WV-37233 | mU*SfU*SmA*SfU*SmA*SfG*SmA*SfG*RmCfAmAmGmAfAm | UUAUAGAGCAAGAACACUGUU | SSSSSSSROOOOOOOOOOO |
| | CfAmCfUmGfUmU*SmU*SmU | UU | OSS |
| WV-37234 | mU*SfU*SmA*SfU*SmA*SfG*SmA*SfG*SmCfAmAmGmAfAm | UUAUAGAGCAAGAACACUGUU | SSSSSSSSOOOOOOOOOOO |
| | CfAmCfUmGfUmU*SmU*SmU | UU | OSS |
| WV-37235 | mU*RfU*RmA*RfU*RmA*RfG*RmA*RfG*RmCfAmAmGmAfAm | UUAUAGAGCAAGAACACUGUU | RRRRRRRROOOOOOOOOOO |
| | CfAmCfUmGfUmU*SmU*SmU | UU | OSS |
| WV-37236 | mU*fU*mA*fU*mA*fG*mA*fG*mCfAmAmGmAfAmCfAmCfUm | UUAUAGAGCAAGAACACUGUU | XXXXXXXXOOOOOOOOOOO |
| | GfUmU*mU*mU | UU | OXX |
| WV-37236 | mU*fU*mA*fU*mA*fG*mA*fG*mCfAmAmGmAfAmCfAmCfUm | UUAUAGAGCAAGAACACUGUU | XXXXXXXXOOOOOOOOOOO |
| | GfUmU*mU*mU | UU | OXX |
| WV-38082 | mUfUmAfUmAfGmAfGmCfAmAmGmAfAmCfAmCfUmGfUmU*mU | UUAUAGAGCAAGAACACUGUU | OOOOOOOOOOOOOOOOOOO |
| | *mU | UU | OXX |
| WV-38083 | mUfUmAfUmAfGmAfGmCfAmAmGmAfAmCfAmCfUmGfUmU*Sm | UUAUAGAGCAAGAACACUGUU | OOOOOOOOOOOOOOOOOOO |
| | U*SmU | UU | OSS |
| WV-38087 | mU*RfUmAfUmAfGmAfGmCfAmAmGmAfAmCfAmCfUmGfUmU* | UUAUAGAGCAAGAACACUGUU | ROOOOOOOOOOOOOOOOOO |
| | SmU*SmU | UU | OSS |
| WV-38088 | mUfU*RmAfUmAfGmAfGmCfAmAmGmAfAmCfAmCfUmGfUmU* | UUAUAGAGCAAGAACACUGUU | OROOOOOOOOOOOOOOOOO |
| | SmU*SmU | UU | OSS |
| WV-38089 | mUfUmA*RfUmAfGmAfGmCfAmAmGmAfAmCfAmCfUmGfUmU* | UUAUAGAGCAAGAACACUGUU | OOROOOOOOOOOOOOOOOO |
| | SmU*SmU | UU | OSS |
| WV-38090 | mUfUmAfU*RmAfGmAfGmCfAmAmGmAfAmCfAmCfUmGfUmU* | UUAUAGAGCAAGAACACUGUU | OOOROOOOOOOOOOOOOOO |
| | SmU*SmU | UU | OSS |
| WV-38091 | mUfUmAfUmA*RfGmAfGmCfAmAmGmAfAmCfAmCfUmGfUmU* | UUAUAGAGCAAGAACACUGUU | OOOOROOOOOOOOOOOOOO |
| | SmU*SmU | UU | OSS |
| WV-38092 | mUfUmAfUmAfG*RmAfGmCfAmAmGmAfAmCfAmCfUmGfUmU* | UUAUAGAGCAAGAACACUGUU | OOOOOROOOOOOOOOOOOO |
| | SmU*SmU | UU | OSS |
| WV-38093 | mUfUmAfUmAfGmA*RfGmCfAmAmGmAfAmCfAmCfUmGfUmU* | UUAUAGAGCAAGAACACUGUU | OOOOOOROOOOOOOOOOOO |
| | SmU*SmU | UU | OSS |
| WV-38094 | mUfUmAfUmAfGmAfG*RmCfAmAmGmAfAmCfAmCfUmGfUmU* | UUAUAGAGCAAGAACACUGUU | OOOOOOOROOOOOOOOOOO |
| | SmU*SmU | UU | OSS |
| WV-38095 | mUfUmAfUmAfGmAfGmC*RfAmAmGmAfAmCfAmCfUmGfUmU* | UUAUAGAGCAAGAACACUGUU | OOOOOOOOROOOOOOOOOO |
| | SmU*SmU | UU | OSS |
| WV-38096 | mUfUmAfUmAfGmAfGmCfA*RmAmGmAfAmCfAmCfUmGfUmU* | UUAUAGAGCAAGAACACUGUU | OOOOOOOOOROOOOOOOOO |
| | SmU*SmU | UU | OSS |
| WV-38097 | mUfUmAfUmAfGmAfGmCfAmA*RmGmAfAmCfAmCfUmGfUmU* | UUAUAGAGCAAGAACACUGUU | OOOOOOOOOOROOOOOOOO |
| | SmU*SmU | UU | OSS |
| WV-38098 | mUfUmAfUmAfGmAfGmCfAmAmG*RmAfAmCfAmCfUmGfUmU* | UUAUAGAGCAAGAACACUGUU | OOOOOOOOOOOROOOOOOO |
| | SmU*SmU | UU | OSS |
| WV-38099 | mUfUmAfUmAfGmAfGmCfAmAmGmA*RfAmCfAmCfUmGfUmU* | UUAUAGAGCAAGAACACUGUU | OOOOOOOOOOOOROOOOOO |
| | SmU*SmU | UU | OSS |
| WV-38100 | mUfUmAfUmAfGmAfGmCfAmAmGmAfA*RmCfAmCfUmGfUmU* | UUAUAGAGCAAGAACACUGUU | OOOOOOOOOOOOOROOOOO |
| | SmU*SmU | UU | OSS |
| WV-38101 | mUfUmAfUmAfGmAfGmCfAmAmGmAfAmC*RfAmCfUmGfUmU* | UUAUAGAGCAAGAACACUGUU | OOOOOOOOOOOOOOROOOO |
| | SmU*SmU | UU | OSS |
| WV-38102 | mUfUmAfUmAfGmAfGmCfAmAmGmAfAmCfA*RmCfUmGfUmU* | UUAUAGAGCAAGAACACUGUU | OOOOOOOOOOOOOOOROOO |
| | SmU*SmU | UU | OSS |
| WV-38103 | mUfUmAfUmAfGmAfGmCfAmAmGmAfAmCfAmC*RfUmGfUmU* | UUAUAGAGCAAGAACACUGUU | OOOOOOOOOOOOOOOOROO |
| | SmU*SmU | UU | OSS |
| WV-38104 | mUfUmAfUmAfGmAfGmCfAmAmGmAfAmCfAmCfU*RmGfUmU* | UUAUAGAGCAAGAACACUGUU | OOOOOOOOOOOOOOOOORO |
| | SmU*SmU | UU | OSS |
| WV-38105 | mUfUmAfUmAfGmAfGmCfAmAmGmAfAmCfAmCfUmG*RfUmU* | UUAUAGAGCAAGAACACUGUU | OOOOOOOOOOOOOOOOOOR |
| | SmU*SmU | UU | OSS |
| WV-38106 | mUfUmAfUmAfGmAfGmCfAmAmGmAfAmCfAmCfUmGfU*RmU* | UUAUAGAGCAAGAACACUGUU | OOOOOOOOOOOOOOOOOOO |
| | SmU*SmU | UU | RSS |
| WV-38107 | mU*SfUmAfUmAfGmAfGmCfAmAmGmAfAmCfAmCfUmGfUmU* | UUAUAGAGCAAGAACACUGUU | SOOOOOOOOOOOOOOOOOO |
| | SmU*SmU | UU | OSS |
| WV-38108 | mUfU*SmAfUmAfGmAfGmCfAmAmGmAfAmCfAmCfUmGfUmU* | UUAUAGAGCAAGAACACUGUU | OSOOOOOOOOOOOOOOOOO |
| | SmU*SmU | UU | OSS |
| WV-38109 | mUfUmA*SfUmAfGmAfGmCfAmAmGmAfAmCfAmCfUmGfUmU* | UUAUAGAGCAAGAACACUGUU | OOSOOOOOOOOOOOOOOOO |
| | SmU*SmU | UU | OSS |
| WV-38110 | mUfUmAfU*SmAfGmAfGmCfAmAmGmAfAmCfAmCfUmGfUmU* | UUAUAGAGCAAGAACACUGUU | OOOOSOOOOOOOOOOOOOO |
| | SmU*SmU | UU | OSS |
| WV-38111 | mUfUmAfUmA*SfGmAfGmCfAmAmGmAfAmCfAmCfUmGfUmU* | UUAUAGAGCAAGAACACUGUU | OOOOSOOOOOOOOOOOOOO |
| | SmU*SmU | UU | OSS |
| WV-38112 | mUfUmAfUmAfG*SmAfGmCfAmAmGmAfAmCfAmCfUmGfUmU* | UUAUAGAGCAAGAACACUGUU | OOOOOSOOOOOOOOOOOOO |
| | SmU*SmU | UU | OSS |
| WV-38113 | mUfUmAfUmAfGmA*SfGmCfAmAmGmAfAmCfAmCfUmGfUmU* | UUAUAGAGCAAGAACACUGUU | OOOOOOSOOOOOOOOOOOO |
| | SmU*SmU | UU | OSS |
| WV-38114 | mUfUmAfUmAfGmAfG*SmCfAmAmGmAfAmCfAmCfUmGfUmU* | UUAUAGAGCAAGAACACUGUU | OOOOOOOSOOOOOOOOOOO |
| | SmU*SmU | UU | OSS |
| WV-38115 | mUfUmAfUmAfGmAfGmC*SfAmAmGmAfAmCfAmCfUmGfUmU* | UUAUAGAGCAAGAACACUGUU | OOOOOOOOSOOOOOOOOOO |
| | SmU*SmU | UU | OSS |
| WV-38116 | mUfUmAfUmAfGmAfGmCfA*SmAmGmAfAmCfAmCfUmGfUmU* | UUAUAGAGCAAGAACACUGUU | OOOOOOOOOOSOOOOOOOO |
| | SmU*SmU | UU | OSS |
| WV-38117 | mUfUmAfUmAfGmAfGmCfAmA*SmGmAfAmCfAmCfUmGfUmU* | UUAUAGAGCAAGAACACUGUU | OOOOOOOOOOSOOOOOOOO |
| | SmU*SmU | UU | OSS |
| WV-38118 | mUfUmAfUmAfGmAfGmCfAmAmG*SmAfAmCfAmCfUmGfUmU* | UUAUAGAGCAAGAACACUGUU | OOOOOOOOOOO5OOOOOOO |
| | SmU*SmU | UU | OSS |
| WV-38119 | mUfUmAfUmAfGmAfGmCfAmAmGmA*SfAmCfAmCfUmGfUmU* | UUAUAGAGCAAGAACACUGUU | OOOOOOOOOOOOSOOOOOO |
| | SmU*SmU | UU | OSS |
| WV-38120 | mUfUmAfUmAfGmAfGmCfAmAmGmAfA*SmCfAmCfUmGfUmU* | UUAUAGAGCAAGAACACUGUU | OOOOOOOOOOOOO$OOOOO |
| | SmU*SmU | UU | OSS |
| WV-38121 | mUfUmAfUmAfGmAfGmCfAmAmGmAfAmC*SfAmCfUmGfUmU* | UUAUAGAGCAAGAACACUGUU | OOOOOOOOOOOOOOSOOOO |
| | SmU*SmU | UU | OSS |
| WV-38122 | mUfUmAfUmAfGmAfGmCfAmAmGmAfAmCfA*SmCfUmGfUmU* | UUAUAGAGCAAGAACACUGUU | OOOOOOOOOOOOOOOSOOO |
| | SmU*SmU | UU | OSS |
| WV-38123 | mUfUmAfUmAfGmAfGmCfAmAmGmAfAmCfAmC*SfUmGfUmU* | UUAUAGAGCAAGAACACUGUU | OOOOOOOOOOOOOOOOSOO |
| | SmU*SmU | UU | OSS |
| WV-38124 | mUfUmAfUmAfGmAfGmCfAmAmGmAfAmCfAmCfU*SmGfUmU* | UUAUAGAGCAAGAACACUGUU | OOOOOOOOOOOOOOOOOSO |
| | SmU*SmU | UU | OSS |
| WV-38125 | mUfUmAfUmAfGmAfGmCfAmAmGmAfAmCfAmCfUmG*SfUmU* | UUAUAGAGCAAGAACACUGUU | OOOOOOOOOOOOOOOOOOS |
| | SmU*SmU | UU | OSS |
| WV-38126 | mUfUmAfUmAfGmAfGmCfAmAmGmAfAmCfAmCfUmGfU*SmU* | UUAUAGAGCAAGAACACUGUU | OOOOOOOOOOOOOOOOOOO |
| | SmU*SmU | UU | SSS |
| WV-38127 | mU*fUmAfUmAfGmAfGmCfAmAmGmAfAmCfAmCfUmGfUmU*S | UUAUAGAGCAAGAACACUGUU | XOOOOOOOOOOOOOOOOOO |
| | mU*SmU | UU | OSS |
| WV-38128 | mUfU*mAfUmAfGmAfGmCfAmAmGmAfAmCfAmCfUmGfUmU*S | UUAUAGAGCAAGAACACUGUU | OXOOOOOOOOOOOOOOOOO |
| | mU*SmU | UU | OSS |
| WV-38129 | mUfUmA*fUmAfGmAfGmCfAmAmGmAfAmCfAmCfUmGfUmU*S | UUAUAGAGCAAGAACACUGUU | OOXOOOOOOOOOOOOOOOO |
| | mU*SmU | UU | OSS |
| WV-38130 | mUfUmAfU*mAfGmAfGmCfAmAmGmAfAmCfAmCfUmGfUmU*S | UUAUAGAGCAAGAACACUGUU | OOOXOOOOOOOOOOOOOOO |
| | mU*SmU | UU | OSS |
| WV-38131 | mUfUmAfUmA*fGmAfGmCfAmAmGmAfAmCfAmCfUmGfUmU*S | UUAUAGAGCAAGAACACUGUU | OOOOXOOOOOOOOOOOOOO |
| | mU*SmU | UU | OSS |
| WV-38132 | mUfUmAfUmAfG*mAfGmCfAmAmGmAfAmCfAmCfUmGfUmU*S | UUAUAGAGCAAGAACACUGUU | OOOOOXOOOOOOOOOOOOO |
| | mU*SmU | UU | OSS |
| WV-38133 | mUfUmAfUmAfGmA*fGmCfAmAmGmAfAmCfAmCfUmGfUmU*S | UUAUAGAGCAAGAACACUGUU | OOOOOOXOOOOOOOOOOOO |
| | mU*SmU | UU | OSS |
| WV-38134 | mUfUmAfUmAfGmAfG*mCfAmAmGmAfAmCfAmCfUmGfUmU*S | UUAUAGAGCAAGAACACUGUU | OOOOOOOXOOOOOOOOOOO |
| | mU*SmU | UU | OSS |
| WV-38135 | mUfUmAfUmAfGmAfGmC*fAmAmGmAfAmCfAmCfUmGfUmU*S | UUAUAGAGCAAGAACACUGUU | OOOOOOOOXOOOOOOOOOO |
| | mU*SmU | UU | OSS |
| WV-38136 | mUfUmAfUmAfGmAfGmCfA*mAmGmAfAmCfAmCfUmGfUmU*S | UUAUAGAGCAAGAACACUGUU | OOOOOOOOOXOOOOOOOOO |
| | mU*SmU | UU | OSS |
| WV-38137 | mUfUmAfUmAfGmAfGmCfAmA*mGmAfAmCfAmCfUmGfUmU*S | UUAUAGAGCAAGAACACUGUU | OOOOOOOOOOXOOOOOOOO |
| | mU*SmU | UU | OSS |
| WV-38138 | mUfUmAfUmAfGmAfGmCfAmAmG*mAfAmCfAmCfUmGfUmU*S | UUAUAGAGCAAGAACACUGUU | OOOOOOOOOOOXOOOOOOO |
| | mU*SmU | UU | OSS |
| WV-38139 | mUfUmAfUmAfGmAfGmCfAmAmGmA*fAmCfAmCfUmGfUmU*S | UUAUAGAGCAAGAACACUGUU | OOOOOOOOOOOOXOOOOOO |
| | mU*SmU | UU | OSS |
| WV-38140 | mUfUmAfUmAfGmAfGmCfAmAmGmAfA*mCfAmCfUmGfUmU*S | UUAUAGAGCAAGAACACUGUU | OOOOOOOOOOOOOXOOOOO |
| | mU*SmU | UU | OSS |
| WV-38141 | mUfUmAfUmAfGmAfGmCfAmAmGmAfAmC*fAmCfUmGfUmU*S | UUAUAGAGCAAGAACACUGUU | OOOOOOOOOOOOOOXOOOO |
| | mU*SmU | UU | OSS |
| WV-38142 | mUfUmAfUmAfGmAfGmCfAmAmGmAfAmCfA*mCfUmGfUmU*S | UUAUAGAGCAAGAACACUGUU | OOOOOOOOOOOOOOOXOOO |
| | mU*SmU | UU | OSS |
| WV-38143 | mUfUmAfUmAfGmAfGmCfAmAmGmAfAmCfAmC*fUmGfUmU*S | UUAUAGAGCAAGAACACUGUU | OOOOOOOOOOOOOOOOXOO |
| | mU*SmU | UU | OSS |
| WV-38144 | mUfUmAfUmAfGmAfGmCfAmAmGmAfAmCfAmCfU*mGfUmU*S | UUAUAGAGCAAGAACACUGUU | OOOOOOOOOOOOOOOOOXO |
| | mU*SmU | UU | OSS |
| WV-38145 | mUfUmAfUmAfGmAfGmCfAmAmGmAfAmCfAmCfUmG*fUmU*S | UUAUAGAGCAAGAACACUGUU | OOOOOOOOOOOOOOOOOOX |
| | mU*SmU | UU | OSS |
| WV-38146 | mUfUmAfUmAfGmAfGmCfAmAmGmAfAmCfAmCfUmGfU*mU*S | UUAUAGAGCAAGAACACUGUU | OOOOOOOOOOOOOOOOOOO |
| | mU*SmU | UU | XSS |
| WV-38678 | mU*fU*mAn001fUmAfGmAfGmCfAn001mAmGmAfAmCfAmCf | UUAUAGAGCAAGAACACUGUU | XXXOOOOOOnXOOOOOOOO |
| | UmGfUmU*mU*mU | UU | OOXX |
| WV-38687 | mU*fU*mAn001fUmAfGmAfGmCfAmAmGmAfAmCfAmCfUmGn | UUAUAGAGCAAGAACACUGUU | XXnXOOOOOOOOOOOOOOO |
| | 001fUmU*mU*mU | UU | nXOXX |
| WV-38703 | mU*RfU*SmAn001fUmAfGmAfGmCfAn001mAmGmAfAmCfAm | UUAUAGAGCAAGAACACUGUU | RSnXOOOOOOnXOOOOOOO |
| | CfUmGfUmU*SmU*SmU | UU | OOOSS |
| WV-38704 | mU*RfU*SmAn001fUmAfGmAfGmCfAn001mAmGmAfAmCfAm | UUAUAGAGCAAGAACACUGUU | RSnXOOOOOOnXOOOOOOO |
| | CfUmGn001fUmU*SmU*SmU | UU | OnXOSS |
| WV-38705 | mU*RfU*SmAn001RfUmAfGmAfGmCfAn001RmAmGmAfAmCf | UUAUAGAGCAAGAACACUGUU | RSnROOOOOOnROOOOOOO |
| | AmCfUmGfUmU*SmU*SmU | UU | OOOSS |
| WV-38706 | mU*RfU*SmAn001RfUmAfGmAfGmCfAn001RmAmGmAfAmCf | UUAUAGAGCAAGAACACUGUU | RSnROOOOOOnROOOOOOO |
| | AmCfUmGn001RfUmU*SmU*SmU | UU | OnROSS |
| WV-38707 | mU*RfU*SmAn001SfUmAfGmAfGmCfAn001SmAmGmAfAmCf | UUAUAGAGCAAGAACACUGUU | RSnSOOOOOOnSOOOOOOO |
| | AmCfUmGfUmU*SmU*SmU | UU | OOOSS |
| WV-40362 | Mod001L001mA*mAfCmAfGmUfGmUfUfCfUmUfGmCfUmCfU | AACAGUGUUCUUGCUCUAUAA | OXOOOOOOOOOOOOOOOOO |
| | mAfUmA*fA | | OX |
| WV-40363 | Mod001L001mA*SmAfCmAfGmUfGmUfUfCfUmUfGmCfUmCf | AACAGUGUUCUUGCUCUAUAA | OSOOOOOOOOOOOOOOOOO |
| | UmAfUmA*SfA | | OS |
| WV-40552 | mU*RfU*SmAn001SfUmAfGmAfGmCfAn001RmAmGmAfAmCf | UUAUAGAGCAAGAACACUGUU | RSnSOOOOOOnROOOOOOO |
| | AmCfUmGn001RfUmU*SmU*SmU | UU | OnROSS |
| WV-40553 | mU*RfU*SmAn001RfUmAfGmAfGmCfAn001RmAmGmAfAmCf | UUAUAGAGCAAGAACACUGUU | RSnROOOOOOnROOOOOOO |
| | AmCfUmGn001SfUmU*SmU*SmU | UU | OnSOSS |
| WV-40555 | mU*RfU*SmAn001RfUmAfGmAfGmCfAn001SmAmGmAfAmCf | UUAUAGAGCAAGAACACUGUU | RSnROOOOOOnSOOOOOOO |
| | AmCfUmGn001SfUmU*SmU*SmU | UU | OnSOSS |
| WV-40556 | mU*RfU*SmAn001SfUmAfGmAfGmCfAn001RmAmGmAfAmCf | UUAUAGAGCAAGAACACUGUU | RSnSOOOOOOnROOOOOOO |
| | AmCfUmGn001SfUmU*SmU*SmU | UU | OnSOSS |
| WV-40796 | mU*RfU*SmAn001RfUmAfGmAfGmCfAn001SmAmGmAfAmCf | UUAUAGAGCAAGAACACUGUU | RSnROOOOOOnSOOOOOOO |
| | AmCfUmGn001RfUmU*SmU*SmU | UU | OnROSS |
| WV-40797 | mU*RfU*SmAn001SfUmAfGmAfGmCfAn001SmAmGmAfAmCf | UUAUAGAGCAAGAACACUGUU | RSnSOOOOOOnSOOOOOOO |
| | AmCfUmGn001RfUmU*SmU*SmU | UU | OnROSS |
| WV-40838 | mU*RfU*SmAn001SfUmAfGmAn001SfGmCfAn001SmAmGmA | UUAUAGAGCAAGAACACUGUU | RSnSOOOnSOOnSOOOOOO |
| | fAmCfAmCfUmGn001SfUmU*SmU*SmU | UU | OOnSOSS |
| WV-40839 | mU*RfU*SmAn001SfUmAfGmAfGn001SmCfAn001SmAmGmA | UUAUAGAGCAAGAACACUGUU | RSnSOOOOnSOnSOOOOOO |
| | fAmCfAmCfUmGn001SfUmU*SmU*SmU | UU | OOnSOSS |
| WV-40842 | mU*RfU*SmAn001SfUmAfGmAn001SfGmCfAn001SmAmGmA | UUAUAGAGCAAGAACACUGUU | RSnSOOOnSOOnSOOOOOn |
| | fAmCfAn001SmCfUmGn001SfUmU*SmU*SmU | UU | SOOnSOSS |
| WV-40843 | mU*RfU*SmAn001SfUmAfGmAfGn001SmCfAn001SmAmGmA | UUAUAGAGCAAGAACACUGUU | RSnSOOOOnSOnSOOOOOn |
| | fAmCfAn001SmCfUmGn001SfUmU*SmU*SmU | UU | SOOnSOSS |
| WV-41896 | mU*RfU*SmAn001RfUmAfGmAfGmCfAmAmGmAfAmCfAmCfU | UUAUAGAGCAAGAACACUGUU | RSnROOOOOOOOOOOOOOO |
| | mGfUmU*SmU*SmU | UU | OOSS |
| WV-41898 | mU*RfU*SmAfUmAn001RfGmAfGmCfAmAmGmAfAmCfAmCfU | UUAUAGAGCAAGAACACUGUU | RSOOnROOOOOOOOOOOOO |
| | mGfUmU*SmU*SmU | UU | OOSS |
| WV-41903 | mU*RfU*SmAfUmAfGmAfGmCfAn001RmAmGmAfAmCfAmCfU | UUAUAGAGCAAGAACACUGUU | RSOOOOOOOnROOOOOOOO |
| | mGfUmU*SmU*SmU | UU | OOSS |
| WV-41912 | mU*RfU*SmAfUmAfGmAfGmCfAmAmGmAfAmCfAmCfUmGn00 | UUAUAGAGCAAGAACACUGUU | RSOOOOOOOOOOOOOOOOn |
| | 1RfUmU*SmU*SmU | UU | ROSS |
| WV-41918 | mU*RfU*SmAn001SfUmAfGmAfGmCfAmAmGmAfAmCfAmCfU | UUAUAGAGCAAGAACACUGUU | RSnSOOOOOOOOOOOOOOO |
| | mGfUmU*SmU*SmU | UU | OOSS |
| WV-41920 | mU*RfU*SmAfUmAn001SfGmAfGmCfAmAmGmAfAmCfAmCfU | UUAUAGAGCAAGAACACUGUU | RSOOnSOOOOOOOOOOOOO |
| | mGfUmU*SmU*SmU | UU | OOSS |
| WV-41925 | mU*RfU*SmAfUmAfGmAfGmCfAn001SmAmGmAfAmCfAmCfU | UUAUAGAGCAAGAACACUGUU | RSOOOOOOOnSOOOOOOOO |
| | mGfUmU*SmU*SmU | UU | OOSS |
| WV-41934 | mU*RfU*SmAfUmAfGmAfGmCfAmAmGmAfAmCfAmCfUmGn00 | UUAUAGAGCAAGAACACUGUU | RSOOOOOOOOOOOOOOOOn |
| | 1SfUmU*SmU*SmU | UU | SOSS |
| WV-41940 | mU*SfU*RmAn001RfUmAfGmAfGmCfAmAmGmAfAmCfAmCfU | UUAUAGAGCAAGAACACUGUU | SRnROOOOOOOOOOOOOOO |
| | mGfUmU*SmU*SmU | UU | OOSS |
| WV-41942 | mU*SfU*RmAfUmAn001RfGmAfGmCfAmAmGmAfAmCfAmCfU | UUAUAGAGCAAGAACACUGUU | SROOnROOOOOOOOOOOOO |
| | mGfUmU*SmU*SmU | UU | OOSS |
| WV-41947 | mU*SfU*RmAfUmAfGmAfGmCfAn001RmAmGmAfAmCfAmCfU | UUAUAGAGCAAGAACACUGUU | SROOOOOOOnROOOOOOOO |
| | mGfUmU*SmU*SmU | UU | OOSS |
| WV-41956 | mU*SfU*RmAfUmAfGmAfGmCfAmAmGmAfAmCfAmCfUmGn00 | UUAUAGAGCAAGAACACUGUU | SROOOOOOOOOOOOOOOOn |
| | 1RfUmU*SmU*SmU | UU | ROSS |
| WV-41962 | mU*SfU*RmAn001SfUmAfGmAfGmCfAmAmGmAfAmCfAmCfU | UUAUAGAGCAAGAACACUGUU | SRnSOOOOOOOOOOOOOOO |
| | mGfUmU*SmU*SmU | UU | OOSS |
| WV-41964 | mU*SfU*RmAfUmAn001SfGmAfGmCfAmAmGmAfAmCfAmCfU | UUAUAGAGCAAGAACACUGUU | SROOnSOOOOOOOOOOOOO |
| | mGfUmU*SmU*SmU | UU | OOSS |
| WV-41969 | mU*SfU*RmAfUmAfGmAfGmCfAn001SmAmGmAfAmCfAmCfU | UUAUAGAGCAAGAACACUGUU | SROOOOOOOnSOOOOOOOO |
| | mGfUmU*SmU*SmU | UU | OOSS |
| WV-41978 | mU*SfU*RmAfUmAfGmAfGmCfAmAmGmAfAmCfAmCfUmGn00 | UUAUAGAGCAAGAACACUGUU | SROOOOOOOOOOOOOOOOn |
| | 1SfUmU*SmU*SmU | UU | SOSS |
| WV-43987 | mU*SfU*RfAn001SmUmAfGmAmGmCfAn001SmAmGmAfAmCf | UUAUAGAGCAAGAACACUGUU | SRnSOOOOOOnSOOOOOOO |
| | AmCmUfGn001SmUmU*SmU*SmU | UU | OnSOSS |
| WV-43990 | mU*SfU*RfAn001SfUmAfGmAmGmCfAn001SfAmGmAfAmCf | UUAUAGAGCAAGAACACUGUU | SRnSOOOOOOnSOOOOOOO |
| | AmCmUfGn001SfUmU*SmU*SmU | UU | OnSOSS |
| WV-43991 | mU*SfU*RmAn001SmUmAfGmAfGmCmAn001SmAmGmAfAmCf | UUAUAGAGCAAGAACACUGUU | SRnSOOOOOOnSOOOOOOO |
| | AmCfUmGn001SmUmU*SmU*SmU | UU | OnSOSS |
| WV-43992 | mU*SfU*RfAn001SmUmAfGmAfGmCfAn001SmAmGmAfAmCf | UUAUAGAGCAAGAACACUGUU | SRnSOOOOOOnSOOOOOOO |
| | AmCfUfGn001SmUmU*SmU*SmU | UU | OnSOSS |
| WV-43993 | mU*SfU*RmAn001SfUmAfGmAfGmCmAn001SfAmGmAfAmCf | UUAUAGAGCAAGAACACUGUU | SRnSOOOOOOnSOOOOOOO |
| | AmCfUmGn001SfUmU*SmU*SmU | UU | OnSOSS |
| WV-49611 | mU*fU*mAfUmAfGmAmGmCmAfAmGmAfAmCfAmCmUmGmUmU* | UUAUAGAGCAAGAACACUGUU | XXOOOOOOOOOOOOOOOOO |
| | mU*mU | UU | OXX |
| WV-49612 | mU*fU*mAn001fUmAfGmAmGmCmAn001fAmGmAfAmCfAmCm | UUAUAGAGCAAGAACACUGUU | XXnXOOOOOOnXOOOOOOO |
| | UmGmUmU*mU*mU | UU | OOOXX |
| WV-49613 | mU*SfC*RmCn001SfUmUfCmCmCmUmGn001SfAmAmGfGmUf | UCCUUCCCUGAAGGUUCCUCC | SRnSOOOOOOnSOOOOOOO |
| | UmCmCmUmCmC*SmU*SmU | UU | OOOSS |
| WV-49614 | mU*SfC*RmCn001SfUmUfCmCmCmUmGn001RfAmAmGfGmUf | UCCUUCCCUGAAGGUUCCUCC | SRnSOOOOOOnROOOOOOO |
| | UmCmCmUmCmC*SmU*SmU | UU | OOOSS |
| WV-49615 | Mod001L001mG*SmGmAmGmGmAfAmCfCfUfUmCmAmGmGmGm | GGAGGAACCUUCAGGGAAGGA | OSOOOOOOOOOOOOOOOOO |
| | AmAmGmG*SmA | | OS |
| WV-49626 | mU*SfU*RmAn001fUmAfGmAmGmCmAn001fAmGmAfAmCfAm | UUAUAGAGCAAGAACACUGUU | SRnXOOOOOOnXOOOOOOO |
| | CmUmGmUmU*SmU*SmU | UU | OOOSS |
| WV-49900 | mU*fC*mCfUmUfCmCfCmUfGmAmAmGfGmUfUmCfCmUfCmC* | UCCUUCCCUGAAGGUUCCUCC | XXOOOOOOOOOOOOOOOOO |
| | mU*mU | UU | OXX |
| WV-49901 | Mod001L001mG*mGfAmGfGmAfAmCfCfUfUmCfAmGfGmGfA | GGAGGAACCUUCAGGGAAGGA | OXOOOOOOOOOOOOOOOOO |
| | mAfGmG*fA | | OX |
| WV-50034 | mU*SfU*RmAn003SfUmAfGmAmGmCmAn003RfAmGmAfAmCf | UUAUAGAGCAAGAACACUGUU | SRnSOOOOOOnROOOOOOO |
| | AmCmUmGmUmU*SmU*SmU | UU | OOOSS |
| WV-50035 | mU*SfU*RmAn003SfUmAfGmAmGmCmAn003SfAmGmAfAmCf | UUAUAGAGCAAGAACACUGUU | SRnSOOOOOOnSOOOOOOO |
| | AmCmUmGmUmU*SmU*SmU | UU | OOOSS |
| WV-50036 | mU*SfU*RmAn004SfUmAfGmAmGmCmAn004RfAmGmAfAmCf | UUAUAGAGCAAGAACACUGUU | SRnSOOOOOOnROOOOOOO |
| | AmCmUmGmUmU*SmU*SmU | UU | OOOSS |
| WV-50037 | mU*SfU*RmAn004SfUmAfGmAmGmCmAn004SfAmGmAfAmCf | UUAUAGAGCAAGAACACUGUU | SRnSOOOOOOnSOOOOOOO |
| | AmCmUmGmUmU*SmU*SmU | UU | OOOSS |
| WV-50040 | mU*SfU*RmAn008SfUmAfGmAmGmCmAn008RfAmGmAfAmCf | UUAUAGAGCAAGAACACUGUU | SRnSOOOOOOnROOOOOOO |
| | AmCmUmGmUmU*SmU*SmU | UU | OOOSS |
| WV-50041 | mU*SfU*RmAn008SfUmAfGmAmGmCmAn008SfAmGmAfAmCf | UUAUAGAGCAAGAACACUGUU | SRnSOOOOOOnSOOOOOOO |
| | AmCmUmGmUmU*SmU*SmU | UU | OOOSS |
| WV-50042 | mU*SfU*RmAn025SfUmAfGmAmGmCmAn025RfAmGmAfAmCf | UUAUAGAGCAAGAACACUGUU | SRnSOOOOOOnROOOOOOO |
| | AmCmUmGmUmU*SmU*SmU | UU | OOOSS |
| WV-50043 | mU*SfU*RmAn025SfUmAfGmAmGmCmAn025SfAmGmAfAmCf | UUAUAGAGCAAGAACACUGUU | SRnSOOOOOOnSOOOOOOO |
| | AmCmUmGmUmU*SmU*SmU | UU | OOOSS |
| WV-50044 | mU*SfU*RmAn026SfUmAfGmAmGmCmAn026RfAmGmAfAmCf | UUAUAGAGCAAGAACACUGUU | SRnSOOOOOOnROOOOOOO |
| | AmCmUmGmUmU*SmU*SmU | UU | OOOSS |
| WV-50045 | mU*SfU*RmAn026SfUmAfGmAmGmCmAn026SfAmGmAfAmCf | UUAUAGAGCAAGAACACUGUU | SRnSOOOOOOnSOOOOOOO |
| | AmCmUmGmUmU*SmU*SmU | UU | OOOSS |
| WV-50046 | mU*SfU*RmAn043SfUmAfGmAmGmCmAn043RfAmGmAfAmCf | UUAUAGAGCAAGAACACUGUU | SRnSOOOOOOnROOOOOOO |
| | AmCmUmGmUmU*SmU*SmU | UU | OOOSS |
| WV-50047 | mU*SfU*RmAn043SfUmAfGmAmGmCmAn043SfAmGmAfAmCf | UUAUAGAGCAAGAACACUGUU | SRnSOOOOOOnSOOOOOOO |
| | AmCmUmGmUmU*SmU*SmU | UU | OOOSS |
| WV-50048 | mU*SfU*RmAn058SfUmAfGmAmGmCmAn058RfAmGmAfAmCf | UUAUAGAGCAAGAACACUGUU | SRnSOOOOOOnROOOOOOO |
| | AmCmUmGmUmU*SmU*SmU | UU | OOOSS |
| WV-50049 | mU*SfU*RmAn058SfUmAfGmAmGmCmAn058SfAmGmAfAmCf | UUAUAGAGCAAGAACACUGUU | SRnSOOOOOOnSOOOOOOO |
| | AmCmUmGmUmU*SmU*SmU | UU | OOOSS |
| WV-50101 | 5mrpmU*RfU*SmAmUmAfGmAmGmCmAmAmGmAfAmCfAmCmUm | UUAUAGAGCAAGAACACUGUU | RSOOOOOOOOOOOOOOOOO |
| | GmUmU*SmU*SmU | UU | OSS |
| WV-50102 | 5mrpmU*SfU*RmAmUmAfGmAmGmCmAmAmGmAfAmCfAmCmUm | UUAUAGAGCAAGAACACUGUU | SROOOOOOOOOOOOOOOOO |
| | GmUmU*SmU*SmU | UU | OSS |
| WV-50103 | 5mrpmU*RfU*SmAn001SfUmAfGmAmGmCmAn001SfAmGmAf | UUAUAGAGCAAGAACACUGUU | RSnSOOOOOOnSOOOOOOO |
| | AmCfAmCmUmGmUmU*SmU*SmU | UU | OOOSS |
| WV-50104 | 5mrpmU*SfU*RmAn001SfUmAfGmAmGmCmAn001SfAmGmAf | UUAUAGAGCAAGAACACUGUU | SRnSOOOOOOnSOOOOOOO |
| | AmCfAmCmUmGmUmU*SmU*SmU | UU | OOOSS |
| WV-50105 | 5mrpmU*RfU*SmAn001SfUmAfGmAmGmCmAn001RfAmGmAf | UUAUAGAGCAAGAACACUGUU | RSnSOOOOOOnROOOOOOO |
| | AmCfAmCmUmGmUmU*SmU*SmU | UU | OOOSS |
| WV-50106 | 5mrpmU*SfU*RmAn001SfUmAfGmAmGmCmAn001RfAmGmAf | UUAUAGAGCAAGAACACUGUU | SRnSOOOOOOnROOOOOOO |
| | AmCfAmCmUmGmUmU*SmU*SmU | UU | OOOSS |
| WV-50108 | 5mvpmU*SfU*RmAmUmAfGmAmGmCmAmAmGmAfAmCfAmCmUm | UUAUAGAGCAAGAACACUGUU | SROOOOOOOOOOOOOOOOO |
| | GmUmU*SmU*SmU | UU | OSS |
| WV-50110 | 5mvpmU*SfU*RmAn001SfUmAfGmAmGmCmAn001SfAmGmAf | UUAUAGAGCAAGAACACUGUU | SRnSOOOOOOnSOOOOOOO |
| | AmCfAmCmUmGmUmU*SmU*SmU | UU | OOOSS |
| WV-50112 | 5mvpmU*SfU*RmAn001SfUmAfGmAmGmCmAn001RfAmGmAf | UUAUAGAGCAAGAACACUGUU | SRnSOOOOOOnROOOOOOO |
| | AmCfAmCmUmGmUmU*SmU*SmU | UU | OOOSS |
| WV-50113 | mU*SfU*RmAn001SfUmAfGmAmGmCmAn009RfAmGmAfAmCf | UUAUAGAGCAAGAACACUGUU | SRnSOOOOOOnROOOOOOO |
| | AmCmUmGmUmU*SmU*SmU | UU | OOOSS |
| WV-50114 | mU*SfU*RmAn001SfUmAfGmAmGmCmAn009SfAmGmAfAmCf | UUAUAGAGCAAGAACACUGUU | SRnSOOOOOOnSOOOOOOO |
| | AmCmUmGmUmU*SmU*SmU | UU | OOOSS |
| WV-50115 | mU*SfU*RmAn001SfUmAfGmAmGmCmAn033RfAmGmAfAmCf | UUAUAGAGCAAGAACACUGUU | SRnSOOOOOOnROOOOOOO |
| | AmCmUmGmUmU*SmU*SmU | UU | OOOSS |
| WV-50116 | mU*SfU*RmAn001SfUmAfGmAmGmCmAn033SfAmGmAfAmCf | UUAUAGAGCAAGAACACUGUU | SRnSOOOOOOnSOOOOOOO |
| | AmCmUmGmUmU*SmU*SmU | UU | OOOSS |
| WV-50481 | mU*fU*mAn001fUmAfGmAmGmCmAn001fAmGmAfAmCfAmCm | UUAUAGAGCAAGAACACUGUU | XXnXOOOOOOnXOOOOOOO |
| | UmGn001fUmU*mU*mU | UU | OnXOXX |
| WV-50482 | mU*fU*mAn001fUmAfGmAmGmCfAn001mAmGmAfAmCfAmCm | UUAUAGAGCAAGAACACUGUU | XXnXOOOOOOnXOOOOOOO |
| | UmGn001fUmU*mU*mU | UU | OnXOXX |
| WV-50485 | mU*SfU*RmAn001fUmAfGmAmGmCmAn001fAmGmAfAmCfAm | UUAUAGAGCAAGAACACUGUU | SRnXOOOOOOnXOOOOOOO |
| | CmUmGn001fUmU*SmU*SmU | UU | OnXOSS |
| WV-50486 | mU*SfU*RmAn001fUmAfGmAmGmCfAn001mAmGmAfAmCfAm | UUAUAGAGCAAGAACACUGUU | SRnXOOOOOOnXOOOOOOO |
| | CmUmGn001fUmU*SmU*SmU | UU | OnXOSS |
| WV-51122 | mU*SfU*RmAfUmAfGmAmGmCmAfAmGmAfAmCfAmCmUmGmUm | UUAUAGAGCAAGAACACUGUU | SROOOOOOOOOOOOOOOOO |
| | U*SmU*SmU | UU | OSS |

TABLE 1a
Example Oligonucleotides/Compositions for non-targeting controls.

| | | | Stereochemistry/ |
|---|---|---|---|
| ID | HELM | Naked Sequence | linkage |
| WV- | mU*SfC*RmCn001SfUmUfCmCmCmUmGn001SfAmAmGfGmU | UCCUUCCCUGAAGGUUCCUCCU | SRnSOOOOOOnSOOOOOOOO |
| 49613 | fUmCmCmUmCmC*SmU*SmU | U | OOSS |
| WV- | mU*SfC*RmCn001SfUmUfCmCmCmUmGn001RfAmAmGfGmU | UCCUUCCCUGAAGGUUCCUCCU | SRnSOOOOOOnROOOOOOO |
| 49614 | fUmCmCmUmCmC*SmU*SmU | U | OOOSS |
| WV- | Mod001L001mG*SmGmAmGmGmAfAmCfCfUfUmCmAmGmG | GGAGGAACCUUCAGGGAAGGA | OSOOOOOOOOOOOOOOOO |
| 49615 | mGmAmAmGmG*SmA | | OOS |
| WV- | mU*fC*mCfUmUfCmCfCmUfGmAmAmGfGmUfUmCfCmUfCm | UCCUUCCCUGAAGGUUCCUCCU | XXOOOOOOOOOOOOOOOO |
| 49900 | C*mU*mU | U | OOXX |
| WV- | Mod001L001mG*mGfAmGfGmAfAmCfCfUfUmCfAmGfGm | GGAGGAACCUUCAGGGAAGGA | OXOOOOOOOOOOOOOOOO |
| 49901 | GfAmAfGmG*fA | | OOX |
| WV- | mU*fC*mCmUmUfCmCmCmUmGmAmAmGfGmUfUmCmCmU | UCCUUCCCUGAAGGUUCCUCCU | XXOOOOOOOOOOOOOOOO |
| 49903 | mCmC*mU*mU | U | OOXX |
| WV- | Mod001L001mG*mGmAmGmGmAfAmCfCfUfUmCmAmGmG | GGAGGAACCUUCAGGGAAGGA | OXOOOOOOOOOOOOOOOO |
| 49904 | mGmAmAmGmG*mA | | OOX |

TABLE 1b
Example Oligonucleotides/Compositions that target TTR.

| ID | HELM | Naked Sequence |
|---|---|---|
| SSR- | RNA1{m(U)[Ssp].[fl2r](U)[Rsp].m(A)[n001S].[fl2r](U)p.m(A)p.[fl2r](G)p.m(A)p.m(G)p.m(C)p.m(A)[n001S].[fl2r](A) | UUAUAGAGCA |
| 0106266 | p.m(G)[n001S].m(A)[n001S].[fl2r](A)p.m(C)p.[fl2r](A)p.m(C)p.m(U)p.m(G)p.m(U)p.m(U)[Ssp].m(U)[Ssp].m(U)}$$ | AGAACACUGU |
| | $$V2.0 | UUU |
| SSR- | RNA1{m(U)[Ssp].[fl2r](U)[Rsp].m(A)[n001S].[fl2r](U)p.m(A)p.[fl2r](G)p.m(A)p.m(G)p.m(C)p.m(A)[n001S].[fl2r](A) | UUAUAGAGCA |
| 0106267 | p.m(G)p.m(A)[n001S].[fl2r](A)[n001S].m(C)p.[fl2r](A)p.m(C)p.m(U)p.m(G)p.m(U)p.m(U)[Ssp].m(U)[Ssp].m(U)}$$ | AGAACACUGU |
| | $$V2.0 | UUU |
| SSR- | RNA1{m(U)[Ssp].[fl2r](U)[Rsp].m(A)[n001S].[fl2r](U)p.m(A)p.[fl2r](G)p.m(A)p.m(G)p.m(C)p.m(A)[n001S].[fl2r](A) | UUAUAGAGCA |
| 0106268 | p.m(G)p.m(A)p.[fl2r](A)[n001S].m(C)[n001S].[fl2r](A)p.m(C)p.m(U)p.m(G)p.m(U)p.m(U)[Ssp].m(U)[Ssp].m(U)}$$ | AGAACACUGU |
| | $$V2.0 | UUU |
| SSR- | RNA1{m(U)[Ssp].[fl2r](U)[Rsp].m(A)[n001S].[fl2r](U)p.m(A)p.[fl2r](G)p.m(A)p.m(G)p.m(C)p.m(A)[n001S].[fl2r](A) | UUAUAGAGCA |
| 0106269 | p.m(G)p.m(A)p.[fl2r](A)p.m(C)[n001S].[fl2r](A)[n001S].m(C)p.m(U)p.m(G)p.m(U)p.m(U)[Ssp].m(U)[Ssp].m(U)}$$ | AGAACACUGU |
| | $$V2.0 | UUU |
| SSR- | RNA1{m(U)[Ssp].[fl2r](U)[Rsp].m(A)[n001S].[fl2r](U)p.m(A)p.[fl2r](G)p.m(A)p.m(G)p.m(C)p.m(A)[n001S].[fl2r](A) | UUAUAGAGCA |
| 0106270 | p.m(G)p.m(A)p.[fl2r](A)p.m(C)p.[fl2r](A)[n001S].m(C)[n001S].m(U)p.m(G)p.m(U)p.m(U)[Ssp].m(U)[Ssp].m(U)}$$ | AGAACACUGU |
| | $$V2.0 | UUU |
| SSR- | RNA1{m(U)[Ssp].[fl2r](U)[Rsp].m(A)[n001S].[fl2r](U)p.m(A)p.[fl2r](G)p.m(A)p.m(G)p.m(C)p.m(A)[n001S].[fl2r](A) | UUAUAGAGCA |
| 0106271 | p.m(G)p.m(A)p.[fl2r](A)p.m(C)p.[fl2r](A)p.m(C)[n001S].m(U)[n001S].m(G)p.m(U)p.m(U)[Ssp].m(U)[Ssp].m(U)}$$ | AGAACACUGU |
| | $$V2.0 | UUU |
| SSR- | RNA1{m(U)[Ssp].[fl2r](U)[Rsp].m(A)[n001S].[fl2r](U)p.m(A)p.[fl2r](G)p.m(A)p.m(G)p.m(C)p.m(A)[n001S].[fl2r](A) | UUAUAGAGCA |
| 0106272 | p.m(G)p.m(A)p.[fl2r](A)p.m(C)p.[fl2r](A)p.m(C)p.m(U)[n001S].m(G)[n001S].m(U)p.m(U)[Ssp].m(U)[Ssp].m(U)}$$ | AGAACACUGU |
| | $$V2.0 | UUU |
| SSR- | RNA1{m(U)[Ssp].[fl2r](U)[Rsp].m(A)[n001S].[fl2r](U)p.m(A)p.[fl2r](G)p.m(A)p.m(G)p.m(C)p.m(A)[n001S].[fl2r](A) | UUAUAGAGCA |
| 0106273 | p.m(G)p.m(A)p.[fl2r](A)p.m(C)p.[fl2r](A)p.m(C)p.m(U)p.m(G)[n001S].m(U)[n001S].m(U)[Ssp].m(U)[Ssp].m(U)}$$ | AGAACACUGU |
| | $$V2.0 | UUU |
| SSR- | RNA1{m(U)[Ssp].[fl2r](U)[Rsp].m(A)[n001S].[fl2r](U)p.m(A)p.[fl2r](G)p.m(A)p.m(G)p.m(C)p.m(A)[n001S].[fl2r](A) | UUAUAGAGCA |
| 0106274 | p.m(G)[n001S].m(A)[n001S].[fl2r](A)[n001S].m(C)p.[fl2r](A)p.m(C)p.m(U)p.m(G)p.m(U)p.m(U)[Ssp].m(U)[Ssp]. | AGAACACUGU |
| | m(U)}$$$$V2.0 | UUU |
| SSR- | RNA1{m(U)[Ssp].[fl2r](U)[Rsp].m(A)[n001S].[fl2r](U)p.m(A)p.[fl2r](G)p.m(A)p.m(G)p.m(C)p.m(A)[n001S].[fl2r](A) | UUAUAGAGCA |
| 0106275 | p.m(G)p.m(A)[n001S].[fl2r](A)[n001S].m(C)[n001S].[fl2r](A)p.m(C)p.m(U)p.m(G)p.m(U)p.m(U)[Ssp].m(U)[Ssp]. | AGAACACUGU |
| | m(U)}$$$$V2.0 | UUU |
| SSR- | RNA1{m(U)[Ssp].[fl2r](U)[Rsp].m(A)[n001S].[fl2r](U)p.m(A)p.[fl2r](G)p.m(A)p.m(G)p.m(C)p.m(A)[n001S].[fl2r](A) | UUAUAGAGCA |
| 0106276 | p.m(G)p.m(A)p.[fl2r](A)[n001S].m(C)[n001S].[fl2r](A)[n001S].m(C)p.m(U)p.m(G)p.m(U)p.m(U)[Ssp].m(U)[Ssp]. | AGAACACUGU |
| | m(U)}$$$$V2.0 | UUU |
| SSR- | RNA1{m(U)[Ssp].[fl2r](U)[Rsp].m(A)[n001S].[fl2r](U)p.m(A)p.[fl2r](G)p.m(A)p.m(G)p.m(C)p.m(A)[n001S].[fl2r](A) | UUAUAGAGCA |
| 0106277 | p.m(G)p.m(A)p.[fl2r](A)p.m(C)[n001S].[fl2r](A)[n001S].m(C)[n001S].m(U)p.m(G)p.m(U)p.m(U)[Ssp].m(U)[Ssp]. | AGAACACUGU |
| | m(U)}$$$$V2.0 | UUU |
| SSR- | RNA1{m(U)[Ssp].[fl2r](U)[Rsp].m(A)[n001S].[fl2r](U)p.m(A)p.[fl2r](G)p.m(A)p.m(G)p.m(C)p.m(A)[n001S].[fl2r](A) | UUAUAGAGCA |
| 0106278 | p.m(G)p.m(A)p.[fl2r](A)p.m(C)p.[fl2r](A)[n001S].m(C)[n001S].m(U)[n001S].m(G)p.m(U)p.m(U)[Ssp].m(U)[Ssp]. | AGAACACUGU |
| | m(U)}$$$$V2.0 | UUU |
| SSR- | RNA1{m(U)[Ssp].[fl2r](U)[Rsp].m(A)[n001S].[fl2r](U)p.m(A)p.[fl2r](G)p.m(A)p.m(G)p.m(C)p.m(A)[n001S].[fl2r](A) | UUAUAGAGCA |
| 0106279 | p.m(G)p.m(A)p.[fl2r](A)p.m(C)p.[fl2r](A)p.m(C)[n001S].m(U)[n001S].m(G)[n001S].m(U)p.m(U)[Ssp].m(U)[Ssp]. | AGAACACUGU |
| | m(U)}$$$$V2.0 | UUU |
| SSR- | RNA1{m(U)[Ssp].[fl2r](U)[Rsp].m(A)[n001S].[fl2r](U)p.m(A)p.[fl2r](G)p.m(A)p.m(G)p.m(C)p.m(A)[n001S].[fl2r](A) | UUAUAGAGCA |
| 0106280 | p.m(G)p.m(A)p.[fl2r](A)p.m(C)p.[fl2r](A)p.m(C)p.m(U)[n001S].m(G)[n001S].m(U)[n001S].m(U)[Ssp].m(U)[Ssp]. | AGAACACUGU |
| | m(U)}$$$$V2.0 | UUU |
| SSR- | RNA1{m(U)[Ssp].[fl2r](U)[Rsp].m(A)[n001S].[fl2r](U)p.m(A)p.[fl2r](G)p.m(A)p.m(G)p.m(C)p.m(A)[n001S].[fl2r](A) | UUAUAGAGCA |
| 0106281 | p.m(G)[n001S].m(A)[n001S].[fl2r](A)[n001S].m(C)[n001S].[fl2r](A)p.m(C)p.m(U)p.m(G)p.m(U)p.m(U)[Ssp].m(U)[S | AGAACACUGU |
| | sp].m(U)}$$$$V2.0 | UUU |
| SSR- | RNA1{m(U)[Ssp].[fl2r](U)[Rsp].m(A)[n001S].[fl2r](U)p.m(A)p.[fl2r](G)p.m(A)p.m(G)p.m(C)p.m(A)[n001S].[fl2r](A) | UUAUAGAGCA |
| 0106282 | p.m(G)p.m(A)[n001S].[fl2r](A)[n001S].m(C)[n001S].[fl2r](A)[n001S].m(C)p.m(U)p.m(G)p.m(U)p.m(U)[Ssp].m(U)[S | AGAACACUGU |
| | sp].m(U)}$$$$V2.0 | UUU |
| SSR- | RNA1{m(U)[Ssp].[fl2r](U)[Rsp].m(A)[n001S].[fl2r](U)p.m(A)p.[fl2r](G)p.m(A)p.m(G)p.m(C)p.m(A)[n001S].[fl2r](A) | UUAUAGAGCA |
| 0106283 | p.m(G)p.m(A)p.[fl2r](A)[n001S].m(C)[n001S].[fl2r](A)[n001S].m(C)[n001S].m(U)p.m(G)p.m(U)p.m(U)[Ssp].m(U)[S | AGAACACUGU |
| | sp].m(U)}$$$$V2.0 | UUU |
| SSR- | RNA1{m(U)[Ssp].[fl2r](U)[Rsp].m(A)[n001S].[fl2r](U)p.m(A)p.[fl2r](G)p.m(A)p.m(G)p.m(C)p.m(A)[n001S].[fl2r](A) | UUAUAGAGCA |
| 0106284 | p.m(G)p.m(A)p.[fl2r](A)p.m(C)[n001S].[fl2r](A)[n001S].m(C)[n001S].m(U)[n001S].m(G)p.m(U)p.m(U)[Ssp].m(U)[S | AGAACACUGU |
| | sp].m(U)}$$$$V2.0 | UUU |
| SSR- | RNA1{m(U)[Ssp].[fl2r](U)[Rsp].m(A)[n001S].[fl2r](U)p.m(A)p.[fl2r](G)p.m(A)p.m(G)p.m(C)p.m(A)[n001S].[fl2r](A) | UUAUAGAGCA |
| 0106285 | p.m(G)p.m(A)p.[fl2r](A)p.m(C)p.[fl2r](A)[n001S].m(C)[n001S].m(U)[n001S].m(G)[n001S].m(U)p.m(U)[Ssp].m(U)[S | AGAACACUGU |
| | sp].m(U)}$$$$V2.0 | UUU |
| SSR- | RNA1{m(U)[Ssp].[fl2r](U)[Rsp].m(A)[n001S].[fl2r](U)p.m(A)p.[fl2r](G)p.m(A)p.m(G)p.m(C)p.m(A)[n001S].[fl2r](A) | UUAUAGAGCA |
| 0106286 | p.m(G)p.m(A)p.[fl2r](A)p.m(C)p.[fl2r](A)p.m(C)[n001S].m(U)[n001S].m(G)[n001S].m(U)[n001S].m(U)[Ssp].m(U)[S | AGAACACUGU |
| | sp].m(U)}$$$$V2.0 | UUU |
| SSR- | RNA1{m(U)[Ssp].[fl2r](U)[Rsp].m(A)[n001S].[fl2r](U)p.m(A)p.[fl2r](G)p.m(A)p.m(G)p.m(C)p.m(A)[n001S].[fl2r](A) | UUAUAGAGCA |
| 0106287 | p.m(G)[n001S].m(A)p.[fl2r](A)[n001S].m(C)p.[fl2r](A)[n001S].m(C)p.m(U)[n001S].m(G)p.m(U)[n001S].m(U)[Ssp]. | AGAACACUGU |
| | m(U)[Ssp].m(U)}$$$$V2.0 | UUU |
| SSR- | RNA1{m(U)[Ssp].[fl2r](U)[Rsp].m(A)[n001S].[fl2r](U)p.m(A)p.[fl2r](G)p.m(A)p.m(G)p.m(C)p.m(A)[n001S].[fl2r](A) | UUAUAGAGCA |
| 0106288 | p.m(G)p.m(A)[n001S].[fl2r](A)p.m(C)[n001S].[fl2r](A)p.m(C)[n001S].m(U)p.m(G)[n001S].m(U)p.m(U)[Ssp].m(U)[S | AGAACACUGU |
| | sp].m(U)}$$$$V2.0 | UUU |
| SSR- | RNA1{m(U)[Ssp].[fl2r](U)[Rsp].m(A)[n001S].[fl2r](U)p.m(A)p.[fl2r](G)p.m(A)p.m(G)p.m(C)p.m(A)[n001S].[fl2r](A) | UUAUAGAGCA |
| 0106289 | p.m(G)[n001S].[fl2r](A)p.[fl2r](A)[n001S].[fl2r](C)p.[fl2r](A)[n001S].[fl2r](C)p.m(U)[n001S].[fl2r](G)p.m(U) | AGAACACUGU |
| | [n001S].[fl2r](U)[Ssp].m(U)[Ssp].m(U)}$$$$V2.0 | UUU |
| SSR- | RNA1{m(U)[Ssp].[fl2r](U)[Rsp].m(A)[n001S].[fl2r](U)p.m(A)p.[fl2r](G)p.m(A)p.m(G)p.m(C)p.m(A)[n001S].[fl2r](A) | UUAUAGAGCA |
| 0106290 | p.m(G)p.m(A)[n001S].[fl2r](A)p.m(C)[n001S].[fl2r](A)p.m(C)[n001S].[fl2r](U)p.m(G)[n001S].[fl2r](U)p.m(U)[Ssp]. | AGAACACUGU |
| | m(U)[Ssp].m(U)}$$$$V2.0 | UUU |
| SSR- | RNA1{m(U)[Ssp].[fl2r](U)[Rsp].m(A)[n001S].[fl2r](U)p.m(A)p.[fl2r](G)p.m(A)p.m(G)p.m(C)p.m(A)[n001S].[fl2r](A) | UUAUAGAGCA |
| 0106291 | p.m(G)p.m(A)[n001S].[fl2r](A)[n001S].m(C)p.[fl2r](A)p.m(C)[n001S].m(U)[n001S].m(G)p.m(U)p.m(U)[Ssp].m(U)[S | AGAACACUGU |
| | sp].m(U)}$$$$V2.0 | UUU |
| SSR- | RNA1{m(U)[Ssp].[fl2r](U)[Rsp].m(A)[n001S].[fl2r](U)p.m(A)p.[fl2r](G)p.m(A)p.m(G)p.m(C)p.m(A)[n001S].[fl2r](A) | UUAUAGAGCA |
| 0106292 | p.m(G)p.m(A)p.[fl2r](A)[n001S].m(C)[n001S].[fl2r](A)p.m(C)p.m(U)[n001S].m(G)[n001S].m(U)p.m(U)[Ssp].m(U)[S | AGAACACUGU |
| | sp].m(U)}$$$$V2.0 | UUU |
| SSR- | RNA1{m(U)[Ssp].[fl2r](U)[Rsp].m(A)[n001S].[fl2r](U)p.m(A)p.[fl2r](G)p.m(A)p.m(G)p.m(C)p.m(A)[n001S].[fl2r](A) | UUAUAGAGCA |
| 0106293 | p.m(G)[n001S].m(A)p.[fl2r](A)p.m(C)p.[fl2r](A)p.m(C)p.m(U)p.m(G)p.m(U)p.m(U)[Ssp].m(U)[Ssp].m(U)}$$$$V2.0 | AGAACACUGU |
| | | UUU |
| SSR- | RNA1{m(U)[Ssp].[fl2r](U)[Rsp].m(A)[n001S].[fl2r](U)p.m(A)p.[fl2r](G)p.m(A)p.m(G)p.m(C)p.m(A)[n001S].[fl2r](A) | UUAUAGAGCA |
| 0106294 | p.m(G)p.m(A)[n001S].[fl2r](A)p.m(C)p.[fl2r](A)p.m(C)p.m(U)p.m(G)p.m(U)p.m(U)[Ssp].m(U)[Ssp].m(U)}$$$$V2.0 | AGAACACUGU |
| | | UUU |
| SSR- | RNA1{m(U)[Ssp].[fl2r](U)[Rsp].m(A)[n001S].[fl2r](U)p.m(A)p.[fl2r](G)p.m(A)p.m(G)p.m(C)p.m(A)[n001S].[fl2r](A) | UUAUAGAGCA |
| 0106295 | p.m(G)p.m(A)p.[fl2r](A)[n001S].m(C)p.[fl2r](A)p.m(C)p.m(U)p.m(G)p.m(U)p.m(U)[Ssp].m(U)[Ssp].m(U)}$$$$V2.0 | AGAACACUGU |
| | | UUU |
| SSR- | RNA1{m(U)[Ssp].[fl2r](U)[Rsp].m(A)[n001S].[fl2r](U)p.m(A)p.[fl2r](G)p.m(A)p.m(G)p.m(C)p.m(A)[n001S].[fl2r](A) | UUAUAGAGCA |
| 0106296 | p.m(G)p.m(A)p.[fl2r](A)p.m(C)[n001S].[fl2r](A)p.m(C)p.m(U)p.m(G)p.m(U)p.m(U)[Ssp].m(U)[Ssp].m(U)}$$$$V2.0 | AGAACACUGU |
| | | UUU |
| SSR- | RNA1{m(U)[Ssp].[fl2r](U)[Rsp].m(A)[n001S].[fl2r](U)p.m(A)p.[fl2r](G)p.m(A)p.m(G)p.m(C)p.m(A)[n001S].[fl2r](A) | UUAUAGAGCA |
| 0106297 | p.m(G)p.m(A)p.[fl2r](A)p.m(C)p.[fl2r](A)[n001S].m(C)p.m(U)p.m(G)p.m(U)p.m(U)[Ssp].m(U)[Ssp].m(U)}$$$$V2.0 | AGAACACUGU |
| | | UUU |
| SSR- | RNA1{m(U)[Ssp].[fl2r](U)[Rsp].m(A)[n001S].[fl2r](U)p.m(A)p.[fl2r](G)p.m(A)p.m(G)p.m(C)p.m(A)[n001S].[fl2r](A) | UUAUAGAGCA |
| 0106298 | p.m(G)p.m(A)p.[fl2r](A)p.m(C)p.[fl2r](A)p.m(C)[n001S].m(U)p.m(G)p.m(U)p.m(U)[Ssp].m(U)[Ssp].m(U)}$$$$V2.0 | AGAACACUGU |
| | | UUU |
| SSR- | RNA1{m(U)[Ssp].[fl2r](U)[Rsp].m(A)[n001S].[fl2r](U)p.m(A)p.[fl2r](G)p.m(A)p.m(G)p.m(C)p.m(A)[n001S].[fl2r](A) | UUAUAGAGCA |
| 0106299 | p.m(G)p.m(A)p.[fl2r](A)p.m(C)p.[fl2r](A)p.m(C)p.m(U)[n001S].m(G)p.m(U)p.m(U)[Ssp].m(U)[Ssp].m(U)}$$$$V2.0 | AGAACACUGU |
| | | UUU |
| SSR- | RNA1{m(U)[Ssp].[fl2r](U)[Rsp].m(A)[n001S].[fl2r](U)p.m(A)p.[fl2r](G)p.m(A)p.m(G)p.m(C)p.m(A)[n001S].[fl2r](A) | UUAUAGAGCA |
| 0106300 | p.m(G)p.m(A)p.[fl2r](A)p.m(C)p.[fl2r](A)p.m(C)p.m(U)p.m(G)[n001S].m(U)p.m(U)[Ssp].m(U)[Ssp].m(U)}$$$$V2.0 | AGAACACUGU |
| | | UUU |
| SSR- | RNA1{m(U)[Ssp].[fl2r](U)[Rsp].m(A)[n001S].[fl2r](U)p.m(A)p.[fl2r](G)p.m(A)p.m(G)p.m(C)p.m(A)[n001S].[fl2r](A) | UUAUAGAGCA |
| 0106301 | p.m(G)p.m(A)p.[fl2r](A)p.m(C)p.[fl2r](A)p.m(C)p.m(U)p.m(G)p.m(U)[n001S].m(U)[Ssp].m(U)[Ssp].m(U)}$$$$V2.0 | AGAACACUGU |
| | | UUU |
| SSR- | RNA1{m(U)[Ssp].[fl2r](U)[Rsp].m(A)[n001S].[fl2r](U)p.m(A)p.[fl2r](G)p.m(A)p.m(G)p.m(C)p.m(A)[n001S].[fl2r](A) | UUAUAGAGCA |
| 0106302 | p.m(G)[n001S].[fl2r](A)p.[fl2r](A)p.m(C)p.[fl2r](A)p.m(C)p.m(U)p.m(G)p.m(U)p.m(U)[Ssp].m(U)[Ssp].m(U)}$$$$V | AGAACACUGU |
| | 2.0 | UUU |
| SSR- | RNA1{m(U)[Ssp].[fl2r](U)[Rsp].m(A)[n001S].[fl2r](U)p.m(A)p.[fl2r](G)p.m(A)p.m(G)p.m(C)p.m(A)[n001S].[fl2r](A) | UUAUAGAGCA |
| 0106303 | p.m(G)p.m(A)p.[fl2r](A)[n001S].[fl2r](C)p.[fl2r](A)p.m(C)p.m(U)p.m(G)p.m(U)p.m(U)[Ssp].m(U)[Ssp].m(U)}$$$$V | AGAACACUGU |
| | 2.0 | UUU |
| SSR- | RNA1{m(U)[Ssp].[fl2r](U)[Rsp].m(A)[n001S].[fl2r](U)p.m(A)p.[fl2r](G)p.m(A)p.m(G)p.m(C)p.m(A)[n001S].[fl2r](A) | UUAUAGAGCA |
| 0106304 | p.m(G)p.m(A)p.[fl2r](A)p.m(C)p.[fl2r](A)[n001S].[fl2r](C)p.m(U)p.m(G)p.m(U)p.m(U)[Ssp].m(U)[Ssp].m(U)}$$$$V | AGAACACUGU |
| | 2.0 | UUU |
| SSR- | RNA1{m(U)[Ssp].[fl2r](U)[Rsp].m(A)[n001S].[fl2r](U)p.m(A)p.[fl2r](G)p.m(A)p.m(G)p.m(C)p.m(A)[n001S].[fl2r](A) | UUAUAGAGCA |
| 0106305 | p.m(G)p.m(A)p.[fl2r](A)p.m(C)p.[fl2r](A)p.m(C)[n001S].[fl2r](U)p.m(G)p.m(U)p.m(U)[Ssp].m(U)[Ssp].m(U)}$$$$V | AGAACACUGU |
| | 2.0 | UUU |
| SSR- | RNA1{m(U)[Ssp].[fl2r](U)[Rsp].m(A)[n001S].[fl2r](U)p.m(A)p.[fl2r](G)p.m(A)p.m(G)p.m(C)p.m(A)[n001S].[fl2r](A) | UUAUAGAGCA |
| 0106306 | p.m(G)p.m(A)p.[fl2r](A)p.m(C)p.[fl2r](A)p.m(C)p.m(U)[n001S].[fl2r](G)p.m(U)p.m(U)[Ssp].m(U)[Ssp].m(U)}$$$$V | AGAACACUGU |
| | 2.0 | UUU |
| SSR- | RNA1{m(U)[Ssp].[fl2r](U)[Rsp].m(A)[n001S].[fl2r](U)p.m(A)p.[fl2r](G)p.m(A)p.m(G)p.m(C)p.m(A)[n001S].[fl2r](A) | UUAUAGAGCA |
| 0104474 | p.m(G)p.m(A)p.[fl2r](A)p.m(C)p.[fl2r](A)p.m(C)p.m(U)p.m(G)[n001S].[fl2r](U)p.m(U)[Ssp].m(U)[Ssp].m(U)}$$$$V | AGAACACUGU |
| | 2.0 | UUU |
| SSR- | RNA1{m(U)[Ssp].[fl2r](U)[Rsp].m(A)[n001S].[fl2r](U)p.m(A)p.[fl2r](G)p.m(A)p.m(G)p.m(C)p.m(A)[n001S].[fl2r](A) | UUAUAGAGCA |
| 0106307 | p.m(G)p.m(A)p.[fl2r](A)p.m(C)p.[fl2r](A)p.m(C)p.m(U)p.m(G)p.m(U)[n001S].[fl2r](U)[Ssp].m(U)[Ssp].m(U)}$$$$V | AGAACACUGU |
| | 2.0 | UUU |
| SSR- | RNA1{m(U)[Ssp].[fl2r](U)[Rsp].m(A)[n001S].[fl2r](U)p.m(A)p.[fl2r](G)p.m(A)p.m(G)p.m(C)p.m(A)[n001S].[fl2r](A) | UUAUAGAGCA |
| 0104475 | p.m(G)p.m(A)p.[fl2r](A)p.m(C)p.[fl2r](A)p.m(C)p.m(U)p.m(G)p.m(U)p.m(U)[Ssp].m(U)[Ssp].m(U)}$$$$V2.0 | AGAACACUGU |
| | | UUU |
| SSR- | RNA1{m(U)[sp].[fl2r](U)[sp].m(A)p.m(U)p.m(A)p.[fl2r](G)p.m(A)p.m(G)p.m(C)p.m(A)p.m(A)p.m(G)p.m(A)p.[fl2r] | UUAUAGAGCA |
| 0104720 | (A)p.m(C)p.[fl2r](A)p.m(C)p.m(U)p.m(G)p.m(U)p.m(U)[sp].m(U)[sp].m(U)}$$$$V2.0 | AGAACACUGU |
| | | UUU |
| SSR- | RNA1{p.m(A)[Ssp].m(A)p.m(C)p.m(A)p.m(G)p.m(U)p.[fl2r](G)p.m(U)p.[fl2r](U)p.[fl2r](C)p.[fl2r](U)p.m(U)p.m(G)p. | AACAGUGUUC |
| 0101599 | m(C)p.m(U)p.m(C)p.m(U)p.m(A)p.m(U)p.m(A)[Ssp].m(A)}\|CHEM1{[GalNAc3C12oyl]}\|CHEM2{[nC60]}$CHEM2,R | UUGCUCUAUA |
| | NA1,1:R1-1:R1\|CHEM2,CHEM1,1:R2-1:R1$$$V2. | A |
| SSR- | RNA1{p.m(A)[sp].m(A)p.m(C)p.m(A)p.m(G)p.m(U)p.[fl2r](G)p.m(U)p.[fl2r](U)p.[fl2r](C)p.[fl2r](U)p.m(U)p.m(G)p. | AACAGUGUUC |
| 0101596 | m(C)p.m(U)p.m(C)p.m(U)p.m(A)p.m(U)p.m(A)[sp].m(A)}\|CHEM1{[GalNAc3C12oyl]}\|CHEM2{[nC60]}$CHEM2,RN | UUGCUCUAUA |
| | A1,1:R1-1:R1\|CHEM2,CHEM1,1:R2-1:R1$$$V2.0 | A |
| SSR- | RNA1{p.m(U)[sp].m(G)p.m(G)p.m(U)p.m(A)p.m(U)p.[fl2r](C)p.m(A)p.[fl2r](A)p.[fl2r](A)p.[fl2r](A)p.m(C)p.m(C)p. | UGGUAUCAAA |
| 0101630 | m(U)p.m(C)p.m(A)p.m(U)p.m(G)p.m(U)p.m(C)[sp].m(A)}\|CHEM1{[GalNAc3C12oyl]}\|CHEM2{[nC60]}$CHEM2,RN | ACCUCAUGUC |
| | A1,1:R1-1:R1\|CHEM2,CHEM1,1:R2-1:R1$$$V2.0 | A |
| SSR- | RNA1{p.m(U)[Ssp].m(G)p.m(G)p.m(U)p.m(A)p.m(U)p.[fl2r](C)p.m(A)p.[fl2r](A)p.[fl2r](A)p.[fl2r](A)p.m(C)p.m(C)p. | UGGUAUCAAA |
| 0101695 | m(U)p.m(C)p.m(A)p.m(U)p.m(G)p.m(U)p.m(C)[Ssp].m(A)}\|CHEM1{[GalNAc3C12oyl]}\|CHEM2{[nC60]}$CHEM2,R | ACCUCAUGUC |
| | NA1,1:R1-1:R1\|CHEM2,CHEM1,1:R2-1:R1$$$V2.0 | A |
| SSR- | RNA1{m(U)[sp].[fl2r](G)[sp].m(A)p.[fl2r](C)p.m(A)p.[fl2r](U)p.m(G)p.m(A)p.m(G)p.m(G)p.[fl2r](U)p.m(U)p.m(U)p. | UGACAUGAGG |
| 0105101 | [fl2r](U)p.m(G)p.[fl2r](A)p.m(U)p.m(A)p.m(C)p.m(C)p.m(A)[sp].m(U)[sp].m(U)}$$$$V2.0 | UUUUGAUACC |
| | | AUU |
| SSR- | RNA1{m(U)[Ssp].[fl2r](U)[Rsp].m(A)p.[fl2r](U)p.m(A)p.[fl2r](G)p.m(A)p.m(G)p.m(C)p.m(A)p.m(A)p.m(G)p.m(A)p. | UUAUAGAGCA |
| 0106582 | [fl2r](A)p.m(C)p.[fl2r](A)p.m(C)p.m(U)p.m(G)p.m(U)p.m(U)[Ssp].m(U)[Ssp].m(U)}$$$$V2.0 | AGAACACUGU |
| | | UUU |

Note:
SSR-0104474 = WV-43988
SSR-0104475 = WV-47145
SSR-0104720 = WV-41826
SSR-0101599 = WV-42080
SSR-0101596 = WV-41828
SSR-0101630 = WV-42427
SSR-0101695 = WV-42589
SSR-0105101 = WV-49593

TABLE 1c
Example Oligonucleotides/Compositions

| Compound | | Naked | |
|---|---|---|---|
| ID | HELM | Sequence | Name |
| SSR-0101596 | RNA1{p.m(A)[sp].m(A)p.m(C)p.m(A)p.m(G)p.m(U)p.[fl2 | AACAGUGUUC | WV-41828 |
| | r](G)p.m(U)p.[fl2r](U)p.[fl2r](C)p.[fl2r](U)p.m(U) | UUGCUCUAUA | |
| | p.m(G)p.m(C)p.m(U)p.m(C)p.m(U)p.m(A)p.m(U)p.m(A)[s | A | |
| | p].m(A)}\|CHEM1{[GalNAc3C12oyl]}\|CHEM2{[nC6o]}$CHEM | | |
| | 2,RNA1,1:R1-1:R1\|CHEM2,CHEM1,1:R2-1:R1$$$V2.0 | | |
| SSR-0101599 | RNA1{p.m(A)[Ssp].m(A)p.m(C)p.m(A)p.m(G)p.m(U)p.[fl | AACAGUGUUC | WV-42080 |
| | 2r](G)p.m(U)p.[fl2r](U)p.[fl2r](C)p.[fl2r](U)p.m(U | UUGCUCUAUA | |
| | )p.m(G)p.m(C)p.m(U)p.m(C)p.m(U)p.m(A)p.m(U)p.m(A)[ | A | |
| | Ssp].m(A)}\|CHEM1{[GalNAc3C12oyl]}\|CHEM2{[nC6o]}$CH | | |
| | EM2,RNA1,1:R1-1:R1\|CHEM2,CHEM1,1:R2-1:R1$$$V2.0 | | |
| SSR-0104021 | RNA1{m(A)[Ssp].m(G)[Ssp].[fl2r](A)[Ssp].m(U)[Ssp]. | AGAUGCCUGA | WV-48429 |
| | [fl2r](G)[Ssp].m(C)[Ssp].[fl2r](C)[n001R].m(U)p.[f | ACUUGAAUUA | |
| | l2r](G)p.[fl2r](A)p.[fl2r](A)p.m(C)p.[fl2r](U)p.m( | A | |
| | U)p.[fl2r](G)[n001R].m(A)[Ssp].[fl2r](A)[Ssp].m(U) | | |
| | [Ssp].[fl2r](U)[Ssp].m(A)[Ssp].m(A)}$$$$V2.0 | | |
| SSR-0104022 | RNA1{m(A)[Ssp].m(G)[Ssp].m(A)[Ssp].m(U)[Ssp].m(G)[ | AGAUGCCUGA | WV-49628 |
| | Ssp].m(C)[Ssp].[fl2r](C)[n001R].m(U)p.[fl2r](G)p.[ | ACUUGAAUUA | |
| | fl2r](A)p.[fl2r](A)p.m(C)p.m(U)p.m(U)p.m(G)[n001R] | A | |
| | .m(A)[Ssp].m(A)[Ssp].m(U)[Ssp].m(U)[Ssp].m(A)[Ssp] | | |
| | .m(A)}$$$$V2.0 | | |
| SSR-0104024 | RNA1{m(A)[sp].m(G)p.[fl2r](A)p.m(U)p.[fl2r](G)p.m( | AGAUGCCUGA | WV-50665 |
| | C)p.[fl2r](C)p.m(U)p.[fl2r](G)p.[fl2r](A)p.[fl2r]( | ACUUGAAUUA | |
| | A)p.m(C)p.[fl2r](U)p.m(U)p.[fl2r](G)p.m(A)p.[fl2r] | A | |
| | (A)p.m(U)p.[fl2r](U)p.m(A)[sp].[fl2r](A)}$$$$V2.0 | | |
| SSR-0104025 | RNA1{m(A)[sp].m(G)p.m(A)p.m(U)p.m(G)p.m(C)p.[fl2r] | AGAUGCCUGA | WV-50667 |
| | (C)p.m(U)p.[fl2r](G)p.[fl2r](A)p.[fl2r](A)p.m(C)p. | ACUUGAAUUA | |
| | m(U)p.m(U)p.m(G)p.m(A)p.m(A)p.m(U)p.m(U)p.m(A)[sp] | A | |
| | .m(A)}$$$$V2.0 | | |
| SSR-0104026 | RNA1{m(C)[Ssp].m(U)[Ssp].[fl2r](G)[Ssp].m(U)[Ssp]. | CUGUGCUGUA | WV-48435 |
| | [fl2r](G)[Ssp].m(C)[Ssp].[fl2r](U)[n001R].m(G)p.[f | ACACAAGUAG | |
| | l2r](U)p.[fl2r](A)p.[fl2r](A)p.m(C)p.[fl2r](A)p.m( | A | |
| | C)p.[fl2r](A)[n001R].m(A)[Ssp].[fl2r](G)[Ssp].m(U) | | |
| | [Ssp].[fl2r](A)[Ssp].m(G)[Ssp].m(A)}$$$$V2.0 | | |
| SSR-0104027 | RNA1{m(C)[Ssp].m(U)[Ssp].m(G)[Ssp].m(U)[Ssp].m(G)[ | CUGUGCUGUA | WV-49633 |
| | Ssp].m(C)[Ssp].[fl2r](U)[n001R].m(G)p.[fl2r](U)p.[ | ACACAAGUAG | |
| | fl2r](A)p.[fl2r](A)p.m(C)p.m(A)p.m(C)p.m(A)[n001R] | A | |
| | .m(A)[Ssp].m(G)[Ssp].m(U)[Ssp].m(A)[Ssp].m(G)[Ssp] | | |
| | .m(A)}$$$$V2.0 | | |
| SSR-0104028 | RNA1{m(C)[sp].m(U)p.[fl2r](G)p.m(U)p.[fl2r](G)p.m( | CUGUGCUGUA | WV-50669 |
| | C)p.[fl2r](U)p.m(G)p.[fl2r](U)p.[f\|2r](A)p.[fl2r]( | ACACAAGUAG | |
| | A)p.m(C)p.[fl2r](A)p.m(C)p.[fl2r](A)p.m(A)p.[fl2r] | A | |
| | (G)p.m(U)p.[fl2r](A)p.m(G)[sp].[fl2r](A)}$$$$V2.0 | | |
| SSR-0104029 | RNA1{m(C)[sp].m(U)p.m(G)p.m(U)p.m(G)p.m(C)p.[fl2r] | CUGUGCUGUA | WV-50671 |
| | (U)p.m(G)p.[fl2r](U)p.[fl2r](A)p.[fl2r](A)p.m(C)p. | ACACAAGUAG | |
| | m(A)p.m(C)p.m(A)p.m(A)p.m(G)p.m(U)p.m(A)p.m(G)[sp] | A | |
| | .m(A)}$$$$V2.0 | | |
| SSR-0104030 | RNA1{m(G)[Ssp].m(G)[Ssp].[fl2r](A)[Ssp].m(U)[Ssp]. | GGAUGAUUGU | WV-48438 |
| | [fl2r](G)[Ssp].m(A)[Ssp].[fl2r](U)[n001R].m(U)p.[f | ACAGAAUCAU | |
| | l2r](G)p.[fl2r](U)p.[fl2r](A)p.m(C)p.[fl2r](A)p.m( | A | |
| | G)p.[fl2r](A)[n001R].m(A)[Ssp].[fl2r](U)[Ssp].m(C) | | |
| | [Ssp].[fl2r](A)[Ssp].m(U)[Ssp].m(A)}$$$$V2.0 | | |
| SSR-0104031 | RNA1{m(U)[Rsp].[fl2r](A)[Ssp].m(A)[n001S].[fl2r](A | UAAAUGCAUA | WV-48433 |
| | )p.m(U)p.[fl2r](G)p.m(C)[n001S].[fl2r](A)p.m(U)p.[ | GUGAUCAGGA | |
| | fl2r](A)p.m(G)p.m(U)p.m(G)[Ssp].[fl2r](A)[Ssp].m(U | AUU | |
| | )[Ssp].[fl2r](C)[Ssp].m(A)[Ssp].[fl2r](G)[n001S].m | | |
| | (G)[Ssp].[fl2r](A)[Ssp].m(A)[Ssp].m(U)[Ssp].m(U)}$ | | |
| | $$$V2.0 | | |
| SSR-0104035 | RNA1{m(U)[Rsp].[fl2r](A)[Ssp].m(U)[n001S].[fl2r] | UAUGAUUCUG | WV-48439 |
| | (G)p.m(A)p.[fl2r](U)p.m(U)[n001S].[fl2r](C)p.m(U) | UACAAUCAUC | |
| | p.[fl2r](G)p.m(U)p.m(A)p.m(C)[Ssp].[fl2r](A)[Ssp] | CUU | |
| | .m(A)[Ssp].[fl2r](U)[Ssp].m(C)[Ssp].[fl2r](A)[n00 | | |
| | 1S].m(U)[Ssp].[fl2r](C)[Ssp].m(C)[Ssp].m(U)[Ssp]. | | |
| | m(U)}$$$$V2.0 | | |
| SSR-0104036 | RNA1{m(U)[Rsp].[fl2r](C)[Ssp].m(A)[n001S].[fl2r](U | UCAUGUUGAG | WV-48427 |
| | )p.m(G)p.[fl2r](U)p.m(U)[n001S].[fl2r](G)p.m(A)p.[ | UUUACCACAG | |
| | fl2r](G)p.m(U)p.m(U)p.m(U)[Ssp].[fl2r](A)[Ssp].m(C | AUU | |
| | )[Ssp].[fl2r](C)[Ssp].m(A)[Ssp].[fl2r](C)[n001S].m | | |
| | (A)[Ssp].[fl2r](G)[Ssp].m(A)[Ssp].m(U)[Ssp].m(U)}$ | | |
| | $$$V2.0 | | |
| SSR-0104037 | RNA1{m(U)[Rsp].[fl2r](C)[Ssp].m(U)[n001S].[fl2r](A | UCUACUUGUG | WV-48436 |
| | )p.m(C)p.[fl2r](U)p.m(U)[n001S].[fl2r](G)p.m(U)p.[ | UUACAGCACA | |
| | fl2r](G)p.m(U)p.m(U)p.m(A)[Ssp].[fl2r](C)[Ssp].m(A | GUU | |
| | )[Ssp].[fl2r](G)[Ssp].m(C)[Ssp].[fl2r](A)[n001S].m | | |
| | (C)[Ssp].[fl2r](A)[Ssp].m(G)[Ssp].m(U)[Ssp].m(U)}$ | | |
| | $$$V2.0 | | |
| SSR-0104038 | RNA1{m(U)[Rsp].[fl2r](C)[Ssp].m(U)[n001S].[fl2r](A | UCUACUUGUG | WV-49636 |
| | )p.m(C)p.[fl2r](U)p.m(U)p.m(G)p.m(U)p.m(G)[n001R]. | UUACAGCACA | |
| | [fl2r](U)p.m(U)p.m(A)[Ssp].[fl2r](C)[Ssp].m(A)[Ssp | GUU | |
| | ].[fl2r](G)[Ssp].m(C)[Ssp].m(A)[Ssp].m(C)[Ssp].m(A | | |
| | )[Ssp].m(G)[Ssp].m(U)[Ssp].m(U)}$$$$V2.0 | | |
| SSR-0104039 | RNA1{m(U)[Rsp].[fl2r](C)[Ssp].m(U)[n001S].[fl2r](A | UCUACUUGUG | WV-49634 |
| | )p.m(C)p.[fl2r](U)p.m(U)p.m(G)p.m(U)p.m(G)[n001S]. | UUACAGCACA | |
| | [fl2r](U)p.m(U)p.m(A)[Ssp].[fl2r](C)[Ssp].m(A)[Ssp | GUU | |
| | ].[fl2r](G)[Ssp].m(C)[Ssp].m(A)[Ssp].m(C)[Ssp].m(A | | |
| | )[Ssp].m(G)[Ssp].m(U)[Ssp].m(U)}$$$$V2.0 | | |
| SSR-0104043 | RNA1{m(U)[Rsp].[fl2r](G)[Ssp].m(A)[n001S].[fl2r](G | UGAGAAUCUA | WV-48442 |
| | )p.m(A)p.[fl2r](A)p.m(U)[n001S].[fl2r](C)p.m(U)p.[ | UUCAUGCACU | |
| | fl2r](A)p.m(U)p.m(U)p.m(C)[Ssp].[fl2r](A)[Ssp].m(U | AUU | |
| | )[Ssp].[fl2r](G)[Ssp].m(C)[Ssp].[fl2r](A)[n001S].m | | |
| | (C)[Ssp].[fl2r](U)[Ssp].m(A)[Ssp].m(U)[Ssp].m(U)}$ | | |
| | $$$V2.0 | | |
| SSR-0104044 | RNA1{m(U)[Rsp].[fl2r](G)[Ssp].m(A)[n001S].[fl2r](G | UGAGAAUCUA | WV-49641 |
| | )p.m(A)p.[fl2r](A)p.m(U)p.m(C)p.m(U)p.m(A)[n001R]. | UUCAUGCACU | |
| | [fl2r](U)p.m(U)p.m(C)[Ssp].[fl2r](A)[Ssp].m(U)[Ssp | AUU | |
| | ].[fl2r](G)[Ssp].m(C)[Ssp].m(A)[Ssp].m(C)[Ssp].m(U | | |
| | )[Ssp].m(A)[Ssp].m(U)[Ssp].m(U)}$$$$V2.0 | | |
| SSR-0104045 | RNA1{m(U)[Rsp].[fl2r](G)[Ssp].m(A)[n001S].[fl2r](G | UGAGAAUCUA | WV-49639 |
| | )p.m(A)p.[fl2r](A)p.m(U)p.m(C)p.m(U)p.m(A)[n001S]. | UUCAUGCACU | |
| | [fl2r](U)p.m(U)p.m(C)[Ssp].[fl2r](A)[Ssp].m(U)[Ssp | AUU | |
| | ].[fl2r](G)[Ssp].m(C)[Ssp].m(A)[Ssp].m(C)[Ssp].m(U | | |
| | )[Ssp].m(A)[Ssp].m(U)[Ssp].m(U)}$$$$V2.0 | | |
| SSR-0104052 | RNA1{m(U)[Rsp].[fl2r](U)[Ssp].m(A)[n001S].[fl2r](A | UUAAUUCAAG | WV-48430 |
| | )p.m(U)p.[fl2r](U)p.m(C)[n001S].[fl2r](A)p.m(A)p.[ | UUCAGGCAUC | |
| | fl2r](G)p.m(U)p.m(U)p.m(C)[Ssp].[fl2r](A)[Ssp].m(G | UUU | |
| | )[Ssp].[fl2r](G)[Ssp].m(C)[Ssp].[fl2r](A)[n001S].m | | |
| | (U)[Ssp].[fl2r](C)[Ssp].m(U)[Ssp].m(U)[Ssp].m(U)}$ | | |
| | $$$V2.0 | | |
| SSR-0104053 | RNA1{m(U)[Rsp].[fl2r](U)[Ssp].m(A)[n001S].[fl2r](A | UUAAUUCAAG | WV-49631 |
| | )p.m(U)p.[fl2r](U)p.m(C)p.m(A)p.m(A)p.m(G)[n001R]. | UUCAGGCAUC | |
| | [fl2r](U)p.m(U)p.m(C)[Ssp].[fl2r](A)[Ssp].m(G)[Ssp | UUU | |
| | ].[fl2r](G)[Ssp].m(C)[Ssp].m(A)[Ssp].m(U)[Ssp].m(C | | |
| | )[Ssp].m(U)[Ssp].m(U)[Ssp].m(U)}$$$$V2.0 | | |
| SSR-0104054 | RNA1{m(U)[Rsp].[fl2r](U)[Ssp].m(A)[n001S].[fl2r](A | UUAAUUCAAG | WV-49629 |
| | )p.m(U)p.[fl2r](U)p.m(C)p.m(A)p.m(A)p.m(G)[n001S]. | UUCAGGCAUC | |
| | [fl2r](U)p.m(U)p.m(C)[Ssp].[fl2r](A)[Ssp].m(G)[Ssp | UUU | |
| | ].[fl2r](G)[Ssp].m(C)[Ssp].m(A)[Ssp].m(U)[Ssp].m(C | | |
| | )[Ssp].m(U)[Ssp].m(U)[Ssp].m(U)}$$$$V2.0 | | |
| SSR-0104061 | RNA1{m(U)[Ssp].[fl2r](A)[Rsp].m(A)[n001S].[fl2r](A | UAAAUGCAUA | WV-48434 |
| | )p.m(U)p.[fl2r](G)p.m(C)[n001S].[fl2r](A)p.m(U)p.[ | GUGAUCAGGA | |
| | fl2r](A)p.m(G)p.m(U)p.m(G)[Ssp].[fl2r](A)[Ssp].m(U | AUU | |
| | )[Ssp].[fl2r](C)[Ssp].m(A)[Ssp].[fl2r](G)[n001S].m | | |
| | (G)[Ssp].[fl2r](A)[Ssp].m(A)[Ssp].m(U)[Ssp].m(U)}$ | | |
| | $$$V2.0 | | |
| SSR-0104065 | RNA1{m(U)[Ssp].[fl2r](A)[Rsp].m(U)[n001S].[fl2r](G | UAUGAUUCUG | WV-48440 |
| | )p.m(A)p.[fl2r](U)p.m(U)[n001S].[fl2r](C)p.m(U)p.[ | UACAAUCAUC | |
| | fl2r](G)p.m(U)p.m(A)p.m(C)[Ssp].[fl2r](A)[Ssp].m(A | CUU | |
| | )[Ssp].[fl2r](U)[Ssp].m(C)[Ssp].[fl2r](A)[n001S].m | | |
| | (U)[Ssp].[fl2r](C)[Ssp].m(C)[Ssp].m(U)[Ssp].m(U)}$ | | |
| | $$$V2.0 | | |
| SSR-0104066 | RNA1{m(U)[Ssp].[fl2r](C)[Rsp].m(A)[n001S].[fl2r](U | UCAUGUUGAG | WV-48428 |
| | )p.m(G)p.[fl2r](U)p.m(U)[n001S].[fl2r](G)p.m(A)p.[ | UUUACCACAG | |
| | fl2r](G)p.m(U)p.m(U)p.m(U)[Ssp].[fl2r](A)[Ssp].m(C | AUU | |
| | )[Ssp].[fl2r](C)[Ssp].m(A)[Ssp].[fl2r](C)[n001S].m | | |
| | (A)[Ssp].[fl2r](G)[Ssp].m(A)[Ssp].m(U)[Ssp].m(U)}$ | | |
| | $$$V2.0 | | |
| SSR-0104067 | RNA1{m(U)[Ssp].[fl2r](C)[Rsp].m(U)[n001S].[fl2r](A | UCUACUUGUG | WV-48437 |
| | )p.m(C)p.[fl2r](U)p.m(U)[n001S].[fl2r](G)p.m(U)p.[ | UUACAGCACA | |
| | fl2r](G)p.m(U)p.m(U)p.m(A)[Ssp].[fl2r](C)[Ssp].m(A | GUU | |
| | )[Ssp].[fl2r](G)[Ssp].m(C)[Ssp].[fl2r](A)[n001S].m | | |
| | (C)[Ssp].[fl2r](A)[Ssp].m(G)[Ssp].m(U)[Ssp].m(U)}$ | | |
| | $$$V2.0 | | |
| SSR-0104068 | RNA1{m(U)[Ssp].[fl2r](C)[Rsp].m(U)[n001S].[fl2r](A | UCUACUUGUG | WV-49637 |
| | )p.m(C)p.[fl2r](U)p.m(U)p.m(G)p.m(U)p.m(G)[n001R]. | UUACAGCACA | |
| | [fl2r](U)p.m(U)p.m(A)[Ssp].[fl2r](C)[Ssp].m(A)[Ssp | GUU | |
| | ].[fl2r](G)[Ssp].m(C)[Ssp].m(A)[Ssp].m(C)[Ssp].m(A | | |
| | )[Ssp].m(G)[Ssp].m(U)[Ssp].m(U)}$$$$V2.0 | | |
| SSR-0104069 | RNA1{m(U)[Ssp].[fl2r](C)[Rsp].m(U)[n001S].[fl2r](A | UCUACUUGUG | WV-49635 |
| | )p.m(C)p.[fl2r](U)p.m(U)p.m(G)p.m(U)p.m(G)[n001S]. | UUACAGCACA | |
| | [fl2r](U)p.m(U)p.m(A)[Ssp].[fl2r](C)[Ssp].m(A)[Ssp | GUU | |
| | ].[fl2r](G)[Ssp].m(C)[Ssp].m(A)[Ssp].m(C)[Ssp].m(A | | |
| | )[Ssp].m(G)[Ssp].m(U)[Ssp].m(U)}$$$$V2.0 | | |
| SSR-0104073 | RNA1{m(U)[Ssp].[fl2r](G)[Rsp].m(A)[n001S].[fl2r](G | UGAGAAUCUA | WV-48443 |
| | )p.m(A)p.[fl2r](A)p.m(U)[n001S].[fl2r](C)p.m(U)p.[ | UUCAUGCACU | |
| | fl2r](A)p.m(U)p.m(U)p.m(C)[Ssp].[fl2r](A)[Ssp].m(U | AUU | |
| | )[Ssp].[fl2r](G)[Ssp].m(C)[Ssp].[fl2r](A)[n001S].m | | |
| | (C)[Ssp].[fl2r](U)[Ssp].m(A)[Ssp].m(U)[Ssp].m(U)}$ | | |
| | $$$V2.0 | | |
| SSR-0104074 | RNA1{m(U)[Ssp].[fl2r](G)[Rsp].m(A)[n001S].[fl2r](G | UGAGAAUCUA | WV-49642 |
| | )p.m(A)p.[fl2r](A)p.m(U)p.m(C)p.m(U)p.m(A)[n001R]. | UUCAUGCACU | |
| | [fl2r](U)p.m(U)p.m(C)[Ssp].[fl2r](A)[Ssp].m(U)[Ssp | AUU | |
| | ].[fl2r](G)[Ssp].m(C)[Ssp].m(A)[Ssp].m(C)[Ssp].m(U | | |
| | )[Ssp].m(A)[Ssp].m(U)[Ssp].m(U)}$$$$V2.0 | | |
| SSR-0104075 | RNA1{m(U)[Ssp].[fl2r](G)[Rsp].m(A)[n001S].[fl2r](G | UGAGAAUCUA | WV-49640 |
| | )p.m(A)p.[fl2r](A)p.m(U)p.m(C)p.m(U)p.m(A)[n001S]. | UUCAUGCACU | |
| | [fl2r](U)p.m(U)p.m(C)[Ssp].[fl2r](A)[Ssp].m(U)[Ssp | AUU | |
| | ].[fl2r](G)[Ssp].m(C)[Ssp].m(A)[Ssp].m(C)[Ssp].m(U | | |
| | )[Ssp].m(A)[Ssp].m(U)[Ssp].m(U)}$$$$V2.0 | | |
| SSR-0104082 | RNA1{m(U)[Ssp].[fl2r](U)[Rsp].m(A)[n001S].[fl2r](A | UUAAUUCAAG | WV-48431 |
| | )p.m(U)p.[fl2r](U)p.m(C)[n001S].[fl2r](A)p.m(A)p.[ | UUCAGGCAUC | |
| | fl2r](G)p.m(U)p.m(U)p.m(C)[Ssp].[fl2r](A)[Ssp].m(G | UUU | |
| | )[Ssp].[fl2r](G)[Ssp].m(C)[Ssp].[fl2r](A)[n001S].m | | |
| | (U)[Ssp].[fl2r](C)[Ssp].m(U)[Ssp].m(U)[Ssp].m(U)}$ | | |
| | $$$V2.0 | | |
| SSR-0104083 | RNA1{m(U)[Ssp].[fl2r](U)[Rsp].m(A)[n001S].[fl2r](A | UUAAUUCAAG | WV-49632 |
| | )p.m(U)p.[fl2r](U)p.m(C)p.m(A)p.m(A)p.m(G)[n001R]. | UUCAGGCAUC | |
| | [fl2r](U)p.m(U)p.m(C)[Ssp].[fl2r](A)[Ssp].m(G)[Ssp | UUU | |
| | ].[fl2r](G)[Ssp].m(C)[Ssp].m(A)[Ssp].m(U)[Ssp].m(C | | |
| | )[Ssp].m(U)[Ssp].m(U)[Ssp].m(U)}$$$$V2.0 | | |
| SSR-0104084 | RNA1{m(U)[Ssp].[fl2r](U)[Rsp].m(A)[n001S].[fl2r](A | UUAAUUCAAG | WV-49630 |
| | )p.m(U)p.[fl2r](U)p.m(C)p.m(A)p.m(A)p.m(G)[n001S]. | UUCAGGCAUC | |
| | [fl2r](U)p.m(U)p.m(C)[Ssp].[fl2r](A)[Ssp].m(G)[Ssp | UUU | |
| | ].[fl2r](G)[Ssp].m(C)[Ssp].m(A)[Ssp].m(U)[Ssp].m(C | | |
| | )[Ssp].m(U)[Ssp].m(U)[Ssp].m(U)}$$$$V2.0 | | |
| SSR-0104092 | RNA1{m(U)[Ssp].m(A)[Ssp].[fl2r](G)[Ssp].m(U)[Ssp]. | UAGUGCAUGA | WV-48441 |
| | [fl2r](G)[Ssp].m(C)[Ssp].[fl2r](A)[n001R].m(U)p.[f | AUAGAUUCUC | |
| | l2r](G)p.[fl2r](A)p.[fl2r](A)p.m(U)p.[fl2r](A)p.m( | A | |
| | G)p.[fl2r](A)[n001R].m(U)[Ssp].[fl2r](U)[Ssp].m(C) | | |
| | [Ssp].[fl2r](U)[Ssp].m(C)[Ssp].m(A)}$$$$V2.0 | | |
| SSR-0104094 | RNA1{m(U)[Ssp].m(A)[Ssp].m(G)[Ssp].m(U)[Ssp].m(G)[ | UAGUGCAUGA | WV-49638 |
| | Ssp].m(C)[Ssp].[fl2r](A)[n001R].m(U)p.[fl2r](G)p.[ | AUAGAUUCUC | |
| | fl2r](A)p.[fl2r](A)p.m(U)p.m(A)p.m(G)p.m(A)[n001R] | A | |
| | .m(U)[Ssp].m(U)[Ssp].m(C)[Ssp].m(U)[Ssp].m(C)[Ssp] | | |
| | .m(A)}$$$$V2.0 | | |
| SSR-0104097 | RNA1{m(U)[Ssp].m(C)[Ssp].[fl2r](U)[Ssp].m(G)[Ssp]. | UCUGUGGUAA | WV-48426 |
| | [fl2r](U)[Ssp].m(G)[Ssp].[fl2r](G)[n001R].m(U)p.[f | ACUCAACAUG | |
| | l2r](A)p.[fl2r](A)p.[fl2r](A)p.m(C)p.[fl2r](U)p.m( | A | |
| | C)p.[fl2r](A)[n001R].m(A)[Ssp].[fl2r](C)[Ssp].m(A) | | |
| | [Ssp].[fl2r](U)[Ssp].m(G)[Ssp].m(A)}$$$$V2.0 | | |
| SSR-0104099 | RNA1{m(U)[Ssp].m(U)[Ssp].[fl2r](C)[Ssp].m(C)[Ssp]. | UUCCUGAUCA | WV-48432 |
| | [fl2r](U)[Ssp].m(G)[Ssp].[fl2r](A)[n001R].m(U)p.[f | CUAUGCAUUU | |
| | l2r](C)p.[fl2r](A)p.[fl2r](C)p.m(U)p.[fl2r](A)p.m( | A | |
| | U)p.[fl2r](G)[n001R].m(C)[Ssp].[fl2r](A)[Ssp].m(U) | | |
| | [Ssp].[fl2r](U)[Ssp].m(U)[Ssp].m(A)}$$$$V2.0 | | |
| SSR-0104100 | RNA1{m(U)[sp].[fl2r](C)[sp].m(U)p.[fl2r](A)p.m(C)p | UCUACUUGUG | WV-50670 |
| | .[fl2r](U)p.m(U)p.[fl2r](G)p.m(U)p.[fl2r](G)p.m(U) | UUACAGCACA | |
| | p.m(U)p.m(A)p.[fl2r](C)p.m(A)p.[fl2r](G)p.m(C)p.[f | GUU | |
| | l2r](A)p.m(C)p.[fl2r](A)p.m(G)[sp].m(U)[sp].m(U)}$ | | |
| | $$$V2.0 | | |
| SSR-0104101 | RNA1{m(U)[sp].[fl2r](C)[sp].m(U)p.m(A)p.m(C)p.[fl2 | UCUACUUGUG | WV-50672 |
| | r](U)p.m(U)p.m(G)p.m(U)p.m(G)p.m(U)p.m(U)p.m(A)p.[ | UUACAGCACA | |
| | fl2r](C)p.m(A)p.[fl2r](G)p.m(C)p.m(A)p.m(C)p.m(A)p | GUU | |
| | .m(G)[sp].m(U)[sp].m(U)}$$$$V2.0 | | |
| SSR-0104102 | RNA1{m(U)[sp].[fl2r](G)[sp].m(A)p.[fl2r](G)p.m(A)p | UGAGAAUCUA | WV-50674 |
| | .[fl2r](A)p.m(U)p.[fl2r](C)p.m(U)p.[fl2r](A)p.m(U) | UUCAUGCACU | |
| | p.m(U)p.m(C)p.[fl2r](A)p.m(U)p.[fl2r](G)p.m(C)p.[f | AUU | |
| | l2r](A)p.m(C)p.[fl2r](U)p.m(A)[sp].m(U)[sp].m(U)}$ | | |
| | $$$V2.0 | | |
| SSR-0104103 | RNA1{m(U)[sp].[fl2r](G)[sp].m(A)p.m(G)p.m(A)p.[fl2 | UGAGAAUCUA | WV-50676 |
| | r](A)p.m(U)p.m(C)p.m(U)p.m(A)p.m(U)p.m(U)p.m(C)p.[ | UUCAUGCACU | |
| | fl2r](A)p.m(U)p.[fl2r](G)p.m(C)p.m(A)p.m(C)p.m(U)p | AUU | |
| | .m(A)[sp].m(U)[sp].m(U)}$$$$V2.0 | | |
| SSR-0104104 | RNA1{m(U)[sp].[fl2r](U)[sp].m(A)p.[fl2r](A)p.m(U)p | UUAAUUCAAG | WV-50666 |
| | .[fl2r](U)p.m(C)p.[fl2r](A)p.m(A)p.[fl2r](G)p.m(U) | UUCAGGCAUC | |
| | p.m(U)p.m(C)p.[fl2r](A)p.m(G)p.[fl2r](G)p.m(C)p.[f | UUU | |
| | l2r](A)p.m(U)p.[fl2r](C)p.m(U)[sp].m(U)[sp].m(U)}$ | | |
| | $$$V2.0 | | |
| SSR-0104105 | RNA1{m(U)[sp].[fl2r](U)[sp].m(A)p.m(A)p.m(U)p.[fl2 | UUAAUUCAAG | WV-50668 |
| | r](U)p.m(C)p.m(A)p.m(A)p.m(G)p.m(U)p.m(U)p.m(C)p.[ | UUCAGGCAUC | |
| | fl2r](A)p.m(G)p.[fl2r](G)p.m(C)p.m(A)p.m(U)p.m(C)p | UUU | |
| | .m(U)[sp].m(U)[sp].m(U)}$$$$V2.0 | | |
| SSR-0104106 | RNA1{m(U)[sp].m(A)p.[fl2r](G)p.m(U)p.[fl2r](G)p.m( | UAGUGCAUGA | WV-50673 |
| | C)p.[fl2r](A)p.m(U)p.[fl2r](G)p.[fl2r](A)p.[fl2r]( | AUAGAUUCUC | |
| | A)p.m(U)p.[fl2r](A)p.m(G)p.[fl2r](A)p.m(U)p.[fl2r] | A | |
| | (U)p.m(C)p.[fl2r](U)p.m(C)[sp].[fl2r](A)}$$$$V2.0 | | |
| SSR-0104107 | RNA1{m(U)[sp].m(A)p.m(G)p.m(U)p.m(G)p.m(C)p.[fl2r] | UAGUGCAUGA | WV-50675 |
| | (A)p.m(U)p.[fl2r](G)p.[fl2r](A)p.[fl2r](A)p.m(U)p. | AUAGAUUCUC | |
| | m(A)p.m(G)p.m(A)p.m(U)p.m(U)p.m(C)p.m(U)p.m(C)[sp] | A | |
| | .m(A)}$$$$V2.0 | | |
| SSR-0104474 | RNA1{m(U)[Ssp].[fl2r](U)[Rsp].m(A)[n001S].[fl2r](U | UUAUAGAGCA | WV-43988 |
| | )p.m(A)p.[fl2r](G)p.m(A)p.m(G)p.m(C)p.m(A)[n001S]. | AGAACACUGU | |
| | [fl2r](A)p.m(G)p.m(A)p.[fl2r](A)p.m(C)p.[fl2r](A)p | UUU | |
| | .m(C)p.m(U)p.m(G)[n001S].[fl2r](U)p.m(U)[Ssp].m(U) | | |
| | [Ssp].m(U)}$$$$V2.0 | | |
| SSR-0104475 | RNA1{m(U)[Ssp].[fl2r](U)[Rsp].m(A)[n001S].[fl2r](U | UUAUAGAGCA | WV-47145 |
| | )p.m(A)p.[fl2r](G)p.m(A)p.m(G)p.m(C)p.m(A)[n001S]. | AGAACACUGU | |
| | [fl2r](A)p.m(G)p.m(A)p.[fl2r](A)p.m(C)p.[fl2r](A)p | UUU | |
| | .m(C)p.m(U)p.m(G)p.m(U)p.m(U)[Ssp].m(U)[Ssp].m(U)} | | |
| | $$$$V2.0 | | |
| SSR-0104720 | RNA1{m(U)[sp].[fl2r](U)[sp].m(A)p.m(U)p.m(A)p.[fl2 | UUAUAGAGCA | WV-41826 |
| | r](G)p.m(A)p.m(G)p.m(C)p.m(A)p.m(A)p.m(G)p.m(A)p.[ | AGAACACUGU | |
| | fl2r](A)p.m(C)p.[fl2r](A)p.m(C)p.m(U)p.m(G)p.m(U)p | UUU | |
| | .m(U)[sp].m(U)[sp].m(U)}$$$$V2.0 | | |
| SSR-0106170 | RNA1{m(U)[Rsp].[fl2r](C)[Ssp].m(U)[n001S].[fl2r](A | UCUACUUGUG | |
| | )p.m(C)p.[fl2r](U)p.m(U)[n001S].[fl2r](G)p.m(U)p.[ | UUACAGCACA | |
| | fl2r](G)p.m(U)p.m(U)p.m(A)[Ssp].[fl2r](C)[Ssp].m(A | GUU | |
| | )[Ssp].[fl2r](G)[Ssp].m(C)[Ssp].[fl2r](A)[Ssp].m(C | | |
| | )[n001S].[fl2r](A)[Ssp].m(G)[Ssp].m(U)[Ssp].m(U)}$ | | |
| | $$$V2.0 | | |
| SSR-0106171 | RNA1{m(U)[Rsp].[fl2r](C)[Ssp].m(U)[n001S].[fl2r](A | UCUACUUGUG | |
| | )p.m(C)p.[fl2r](U)p.m(U)p.[fl2r](G)p.m(U)p.[fl2r]( | UUACAGCACA | |
| | G)[n001S].m(U)p.m(U)p.m(A)[Ssp].[fl2r](C)[Ssp].m(A | GUU | |
| | )[Ssp].[fl2r](G)[Ssp].m(C)[Ssp].[fl2r](A)[Ssp].m(C | | |
| | )[n001S].[fl2r](A)[Ssp].m(G)[Ssp].m(U)[Ssp].m(U)}$ | | |
| | $$$V2.0 | | |
| SSR-0106172 | RNA1{m(U)[Rsp].[fl2r](C)[Ssp].m(U)[n001S].[fl2r](A | UCUACUUGUG | |
| | )p.m(C)p.[fl2r](U)p.m(U)p.[fl2r](G)p.m(U)p.m(G)[n0 | UUACAGCACA | |
| | 01S].[fl2r](U)p.m(U)p.m(A)[Ssp].[fl2r](C)[Ssp].m(A | GUU | |
| | )[Ssp].[fl2r](G)[Ssp].m(C)[Ssp].[fl2r](A)[Ssp].m(C | | |
| | )[n001S].[fl2r](A)[Ssp].m(G)[Ssp].m(U)[Ssp].m(U)}$ | | |
| | $$$V2.0 | | |
| SSR-0106173 | RNA1{m(U)[Rsp].[fl2r](C)[Ssp].m(U)[n001S].[fl2r](A | UCUACUUGUG | |
| | )p.m(C)p.[fl2r](U)p.m(U)p.m(G)p.m(U)p.m(G)[n001S]. | UUACAGCACA | |
| | [fl2r](U)p.m(U)p.m(A)[Ssp].[fl2r](C)[Ssp].m(A)[Ssp | GUU | |
| | ].[fl2r](G)[Ssp].m(C)[Ssp].m(A)[Ssp].m(C)[n001S].[ | | |
| | fl2r](A)[Ssp].m(G)[Ssp].m(U)[Ssp].m(U)}$$$$V2.0 | | |
| SSR-0106174 | RNA1{m(U)[Rsp].[fl2r](C)[Ssp].m(U)[n001S].[fl2r](A | UCUACUUGUG | |
| | )p.m(C)p.[fl2r](U)p.m(U)[n001S].[fl2r](G)p.m(U)p.[ | UUACAGCACA | |
| | fl2r](G)p.m(U)p.m(U)p.m(A)[Ssp].[fl2r](C)[Ssp].m(A | GUU | |
| | )[Ssp].[fl2r](G)[Ssp].m(C)[Ssp].[fl2r](A)[Ssp].m(C | | |
| | )[Ssp].[fl2r](A)[Ssp].m(G)[Ssp].m(U)[Ssp].m(U)}$$$ | | |
| | $V2.0 | | |
| SSR-0106175 | RNA1{m(U)[Rsp].[fl2r](C)[Ssp].m(U)[n001S].[fl2r](A | UCUACUUGUG | |
| | )p.m(C)p.[fl2r](U)p.m(U)p.[fl2r](G)p.m(U)p.[fl2r]( | UUACAGCACA | |
| | G)[n001S].m(U)p.m(U)p.m(A)[Ssp].[fl2r](C)[Ssp].m(A | GUU | |
| | )[Ssp].[fl2r](G)[Ssp].m(C)[Ssp].[fl2r](A)[Ssp].m(C | | |
| | )[Ssp].[fl2r](A)[Ssp].m(G)[Ssp].m(U)[Ssp].m(U)}$$$ | | |
| | $V2.0 | | |
| SSR-0106176 | RNA1{m(U)[Rsp].[fl2r](C)[Ssp].m(U)[n001S].[fl2r](A | UCUACUUGUG | |
| | )p.m(C)p.[fl2r](U)p.m(U)p.[fl2r](G)p.m(U)p.m(G)[n0 | UUACAGCACA | |
| | 01S].[fl2r](U)p.m(U)p.m(A)[Ssp].[fl2r](C)[Ssp].m(A | GUU | |
| | )[Ssp].[fl2r](G)[Ssp].m(C)[Ssp].[fl2r](A)[Ssp].m(C | | |
| | )[Ssp].[fl2r](A)[Ssp].m(G)[Ssp].m(U)[Ssp].m(U)}$$$ | | |
| | $V2.0 | | |
| SSR-0106177 | RNA1{m(U)[Rsp].[fl2r](C)[Ssp].m(U)[n001S].[fl2r](A | UCUACUUGUG | |
| | )p.m(C)p.[fl2r](U)p.m(U)p.m(G)p.m(U)p.m(G)[n001S]. | UUACAGCACA | |
| | [fl2r](U)p.m(U)p.m(A)[Ssp].[fl2r](C)[Ssp].m(A)[Ssp | GUU | |
| | ].[fl2r](G)[Ssp].m(C)[Ssp].m(A)[Ssp].m(C)[Ssp].[fl | | |
| | 2r](A)[Ssp].m(G)[Ssp].m(U)[Ssp].m(U)}$$$$V2.0 | | |
| SSR-0106178 | RNA1{m(U)[Rsp].[fl2r](C)[Ssp].m(U)[n001S].[fl2r](A | UCUACUUGUG | |
| | )p.m(C)p.[fl2r](U)p.m(U)[n001S].[fl2r](G)p.m(U)p.[ | UUACAGCACA | |
| | fl2r](G)p.m(U)p.m(U)p.m(A)[Ssp].[fl2r](C)[Ssp].m(A | GUU | |
| | )[n001S].[fl2r](G)[Ssp].m(C)[Ssp].[fl2r](A)[n001S] | | |
| | .m(C)[Ssp].[fl2r](A)[Ssp].m(G)[Ssp].m(U)[Ssp].m(U) | | |
| | }$$$$V2.0 | | |
| SSR-0106179 | RNA1{m(U)[Rsp].[fl2r](C)[Ssp].m(U)[n001S].[fl2r](A | UCUACUUGUG | |
| | )p.m(C)p.[fl2r](U)p.m(U)[n001S].[fl2r](G)p.m(U)p.[ | UUACAGCACA | |
| | fl2r](G)p.m(U)p.m(U)p.m(A)[Ssp].[fl2r](C)[Ssp].m(A | GUU | |
| | )[n001S].[fl2r](G)[Ssp].m(C)[Ssp].[fl2r](A)[Ssp].m | | |
| | (C)[n001S].[fl2r](A)[Ssp].m(G)[Ssp].m(U)[Ssp].m(U) | | |
| | }$$$$V2.0 | | |
| SSR-0106180 | RNA1{m(U)[Rsp].[fl2r](C)[Ssp].m(U)[n001S].[fl2r](A | UCUACUUGUG | |
| | )p.m(C)p.[fl2r](U)p.m(U)p.[fl2r](G)p.m(U)p.[fl2r]( | UUACAGCACA | |
| | G)[n001S].m(U)p.m(U)p.m(A)[Ssp].[fl2r](C)[Ssp].m(A | GUU | |
| | )[n001S].[fl2r](G)[Ssp].m(C)[Ssp].[fl2r](A)[Ssp].m | | |
| | (C)[n001S].[fl2r](A)[Ssp].m(G)[Ssp].m(U)[Ssp].m(U) | | |
| | }$$$$V2.0 | | |
| SSR-0106181 | RNA1{m(U)[Rsp].[fl2r](C)[Ssp].m(U)[n001S].[fl2r](A | UCUACUUGUG | |
| | )p.m(C)p.[fl2r](U)p.m(U)p.[fl2r](G)p.m(U)p.m(G)[n0 | UUACAGCACA | |
| | 01S].[fl2r](U)p.m(U)p.m(A)[Ssp].[fl2r](C)[Ssp].m(A | GUU | |
| | )[n001S].[fl2r](G)[Ssp].m(C)[Ssp].[fl2r](A)[Ssp].m | | |
| | (C)[n001S].[fl2r](A)[Ssp].m(G)[Ssp].m(U)[Ssp].m(U) | | |
| | }$$$$V2.0 | | |
| SSR-0106182 | RNA1{m(U)[Rsp].[fl2r](C)[Ssp].m(U)[n001S].[fl2r](A | UCUACUUGUG | |
| | )p.m(C)p.[fl2r](U)p.m(U)p.m(G)p.m(U)p.m(G)[n001S]. | UUACAGCACA | |
| | [fl2r](U)p.m(U)p.m(A)[Ssp].[fl2r](C)[Ssp].m(A)[n00 | GUU | |
| | 1S].[fl2r](G)[Ssp].m(C)[Ssp].m(A)[Ssp].m(C)[n001S] | | |
| | .[fl2r](A)[Ssp].m(G)[Ssp].m(U)[Ssp].m(U)}$$$$V2.0 | | |
| SSR-0106183 | RNA1{p.[Rm5d5m](U)[Rsp].[fl2r](C)[Ssp].m(U)[n001S] | UCUACUUGUG | |
| | .[fl2r](A)p.m(C)p.[fl2r](U)p.m(U)[n001S].[fl2r](G) | UUACAGCACA | |
| | p.m(U)p.[fl2r](G)p.m(U)p.m(U)p.m(A)[Ssp].[fl2r](C) | GUU | |
| | [Ssp].m(A)[Ssp].[fl2r](G)[Ssp].m(C)[Ssp].[fl2r](A) | | |
| | [n001S].m(C)[Ssp].[fl2r](A)[Ssp].m(G)[Ssp].m(U)[Ss | | |
| | p].m(U)}$$$$V2.0 | | |
| SSR-0106184 | RNA1{p.[Rm5d5m](U)[Rsp].[fl2r](C)[Ssp].m(U)[n001S] | UCUACUUGUG | |
| | .[fl2r](A)p.m(C)p.[fl2r](U)p.m(U)[n001S].[fl2r](G) | UUACAGCACA | |
| | p.m(U)p.[fl2r](G)p.m(U)p.m(U)p.m(A)[Ssp].[fl2r](C) | GUU | |
| | [Ssp].m(A)[Ssp].[fl2r](G)[Ssp].m(C)[Ssp].[fl2r](A) | | |
| | [Ssp].m(C)[n001S].[fl2r](A)[Ssp].m(G)[Ssp].m(U)[Ss | | |
| | p].m(U)}$$$$V2.0 | | |
| SSR-0106185 | RNA1{p.[Rm5d5m](U)[Rsp].[fl2r](C)[Ssp].m(U)[n001S] | UCUACUUGUG | |
| | .[fl2r](A)p.m(C)p.[fl2r](U)p.m(U)p.[fl2r](G)p.m(U) | UUACAGCACA | |
| | p.[fl2r](G)[n001S].m(U)p.m(U)p.m(A)[Ssp].[fl2r](C) | GUU | |
| | [Ssp].m(A)[Ssp].[fl2r](G)[Ssp].m(C)[Ssp].[fl2r](A) | | |
| | [Ssp].m(C)[n001S].[fl2r](A)[Ssp].m(G)[Ssp].m(U)[Ss | | |
| | p].m(U)}$$$$V2.0 | | |
| SSR-0106186 | RNA1{p.[Rm5d5m](U)[Rsp].[fl2r](C)[Ssp].m(U)[n001S] | UCUACUUGUG | |
| | .[fl2r](A)p.m(C)p.[fl2r](U)p.m(U)p.[fl2r](G)p.m(U) | UUACAGCACA | |
| | p.m(G)[n001S].[fl2r](U)p.m(U)p.m(A)[Ssp].[fl2r](C) | GUU | |
| | [Ssp].m(A)[Ssp].[fl2r](G)[Ssp].m(C)[Ssp].[fl2r](A) | | |
| | [Ssp].m(C)[n001S].[fl2r](A)[Ssp].m(G)[Ssp].m(U)[Ss | | |
| | p].m(U)}$$$$V2.0 | | |
| SSR-0106187 | RNA1{p.[Rm5d5m](U)[Rsp].[fl2r](C)[Ssp].m(U)[n001S] | UCUACUUGUG | |
| | .[fl2r](A)p.m(C)p.[fl2r](U)p.m(U)p.m(G)p.m(U)p.m(G | UUACAGCACA | |
| | )[n001S].[fl2r](U)p.m(U)p.m(A)[Ssp].[fl2r](C)[Ssp] | GUU | |
| | .m(A)[Ssp].[fl2r](G)[Ssp].m(C)[Ssp].m(A)[Ssp].m(C) | | |
| | [n001S].[fl2r](A)[Ssp].m(G)[Ssp].m(U)[Ssp].m(U)}$$ | | |
| | $$V2.0 | | |
| SSR-0106188 | RNA1{p.[Rm5d5m](U)[Rsp].[fl2r](C)[Ssp].m(U)[n001S] | UCUACUUGUG | |
| | .[fl2r](A)p.m(C)p.[fl2r](U)p.m(U)[n001S].[fl2r](G) | UUACAGCACA | |
| | p.m(U)p.[fl2r](G)p.m(U)p.m(U)p.m(A)[Ssp].[fl2r](C) | GUU | |
| | [Ssp].m(A)[Ssp].[fl2r](G)[Ssp].m(C)[Ssp].[fl2r](A) | | |
| | [Ssp].m(C)[Ssp].[fl2r](A)[Ssp].m(G)[Ssp].m(U)[Ssp] | | |
| | .m(U)}$$$$V2.0 | | |
| SSR-0106189 | RNA1{p.[Rm5d5m](U)[Rsp].[fl2r](C)[Ssp].m(U)[n001S] | UCUACUUGUG | |
| | .[fl2r](A)p.m(C)p.[fl2r](U)p.m(U)p.[fl2r](G)p.m(U) | UUACAGCACA | |
| | p.[fl2r](G)[n001S].m(U)p.m(U)p.m(A)[Ssp].[fl2r](C) | GUU | |
| | [Ssp].m(A)[Ssp].[fl2r](G)[Ssp].m(C)[Ssp].[fl2r](A) | | |
| | [Ssp].m(C)[Ssp].[fl2r](A)[Ssp].m(G)[Ssp].m(U)[Ssp] | | |
| | .m(U)}$$$$V2.0 | | |
| SSR-0106190 | RNA1{p.[Rm5d5m](U)[Rsp].[fl2r](C)[Ssp].m(U)[n001S] | UCUACUUGUG | |
| | .[fl2r](A)p.m(C)p.[fl2r](U)p.m(U)p.[fl2r](G)p.m(U) | UUACAGCACA | |
| | p.m(G)[n001S].[fl2r](U)p.m(U)p.m(A)[Ssp].[fl2r](C) | GUU | |
| | [Ssp].m(A)[Ssp].[fl2r](G)[Ssp].m(C)[Ssp].[fl2r](A) | | |
| | [Ssp].m(C)[Ssp].[fl2r](A)[Ssp].m(G)[Ssp].m(U)[Ssp] | | |
| | .m(U)}$$$$V2.0 | | |
| SSR-0106191 | RNA1{p.[Rm5d5m](U)[Rsp].[fl2r](C)[Ssp].m(U)[n001S] | UCUACUUGUG | |
| | .[fl2r](A)p.m(C)p.[fl2r](U)p.m(U)p.m(G)p.m(U)p.m(G | UUACAGCACA | |
| | )[n001S].[fl2r](U)p.m(U)p.m(A)[Ssp].[fl2r](C)[Ssp] | GUU | |
| | .m(A)[Ssp].[fl2r](G)[Ssp].m(C)[Ssp].m(A)[Ssp].m(C) | | |
| | [Ssp].[fl2r](A)[Ssp].m(G)[Ssp].m(U)[Ssp].m(U)}$$$$ | | |
| | V2.0 | | |
| SSR-0106192 | RNA1{p.[Rm5d5m](U)[Rsp].[fl2r](C)[Ssp].m(U)[n001S] | UCUACUUGUG | |
| | .[fl2r](A)p.m(C)p.[fl2r](U)p.m(U)p.m(G)p.m(U)p.m(G | UUACAGCACA | |
| | )[n001S].[fl2r](U)p.m(U)p.m(A)[Ssp].[fl2r](C)[Ssp] | GUU | |
| | .m(A)[Ssp].[fl2r](G)[Ssp].m(C)[Ssp].m(A)[Ssp].m(C) | | |
| | [Ssp].m(A)[Ssp].m(G)[Ssp].m(U)[Ssp].m(U)}$$$$V2.0 | | |
| SSR-0106193 | RNA1{p.[Rm5d5m](U)[Rsp].[fl2r](C)[Ssp].m(U)[n001S] | UCUACUUGUG | |
| | .[fl2r](A)p.m(C)p.[fl2r](U)p.m(U)[n001S].[fl2r](G) | UUACAGCACA | |
| | p.m(U)p.[fl2r](G)p.m(U)p.m(U)p.m(A)[Ssp].[fl2r](C) | GUU | |
| | [Ssp].m(A)[n001S].[fl2r](G)[Ssp].m(C)[Ssp].[fl2r]( | | |
| | A)[n001S].m(C)[Ssp].[fl2r](A)[Ssp].m(G)[Ssp].m(U)[ | | |
| | Ssp].m(U)}$$$$V2.0 | | |
| SSR-0106194 | RNA1{p.[Rm5d5m](U)[Rsp].[fl2r](C)[Ssp].m(U)[n001S] | UCUACUUGUG | |
| | .[fl2r](A)p.m(C)p.[fl2r](U)p.m(U)[n001S].[fl2r](G) | UUACAGCACA | |
| | p.m(U)p.[fl2r](G)p.m(U)p.m(U)p.m(A)[Ssp].[fl2r](C) | GUU | |
| | [Ssp].m(A)[n001S].[fl2r](G)[Ssp].m(C)[Ssp].[fl2r]( | | |
| | A)[Ssp].m(C)[n001S].[fl2r](A)[Ssp].m(G)[Ssp].m(U)[ | | |
| | Ssp].m(U)}$$$$V2.0 | | |
| SSR-0106195 | RNA1{p.[Rm5d5m](U)[Rsp].[fl2r](C)[Ssp].m(U)[n001S] | UCUACUUGUG | |
| | .[fl2r](A)p.m(C)p.[fl2r](U)p.m(U)p.[fl2r](G)p.m(U) | UUACAGCACA | |
| | p.[fl2r](G)[n001S].m(U)p.m(U)p.m(A)[Ssp].[fl2r](C) | GUU | |
| | [Ssp].m(A)[n001S].[fl2r](G)[Ssp].m(C)[Ssp].[fl2r]( | | |
| | A)[Ssp].m(C)[n001S].[fl2r](A)[Ssp].m(G)[Ssp].m(U)[ | | |
| | Ssp].m(U)}$$$$V2.0 | | |
| SSR-0106196 | RNA1{p.[Rm5d5m](U)[Rsp].[fl2r](C)[Ssp].m(U)[n001S] | UCUACUUGUG | |
| | .[fl2r](A)p.m(C)p.[fl2r](U)p.m(U)p.[fl2r](G)p.m(U) | UUACAGCACA | |
| | p.m(G)[n001S].[fl2r](U)p.m(U)p.m(A)[Ssp].[fl2r](C) | GUU | |
| | [Ssp].m(A)[n001S].[fl2r](G)[Ssp].m(C)[Ssp].[fl2r]( | | |
| | A)[Ssp].m(C)[n001S].[fl2r](A)[Ssp].m(G)[Ssp].m(U)[ | | |
| | Ssp].m(U)}$$$$V2.0 | | |
| SSR-0106197 | RNA1{p.[Rm5d5m](U)[Rsp].[fl2r](C)[Ssp].m(U)[n001S] | UCUACUUGUG | |
| | .[fl2r](A)p.m(C)p.[fl2r](U)p.m(U)p.m(G)p.m(U)p.m(G | UUACAGCACA | |
| | )[n001S].[fl2r](U)p.m(U)p.m(A)[Ssp].[fl2r](C)[Ssp] | GUU | |
| | .m(A)[n001S].[fl2r](G)[Ssp].m(C)[Ssp].m(A)[Ssp].m( | | |
| | C)[n001S].[fl2r](A)[Ssp].m(G)[Ssp].m(U)[Ssp].m(U)} | | |
| | $$$$V2.0 | | |
| SSR-0106206 | RNA1{m(C)[Ssp].m(U)p.[fl2r](G)p.m(U)p.[fl2r](G)p.m | CUGUGCUGUA | |
| | (C)p.[fl2r](U)[n001R].m(G)p.[fl2r](U)p.[fl2r](A)p. | ACACAAGUAG | |
| | [fl2r](A)p.m(C)p.[fl2r](A)p.m(C)p.[fl2r](A)[n001R] | A | |
| | .m(A)p.[fl2r](G)p.m(U)p.[fl2r](A)p.m(G)[Ssp].m(A)} | | |
| | $$$$V2.0 | | |
| SSR-0106207 | RNA1{m(C)[Ssp].m(U)p.[fl2r](G)p.m(U)p.[fl2r](G)p.m | CUGUGCUGUA | |
| | (C)p.[fl2r](U)[n001R].m(G)p.[fl2r](U)p.[fl2r](A)p. | ACACAAGUAG | |
| | [fl2r](A)p.m(C)p.[fl2r](A)p.m(C)p.[fl2r](A)[n001R] | A | |
| | .m(A)[Ssp].[fl2r](G)[Ssp].m(U)[Ssp].[fl2r](A)[Ssp] | | |
| | .m(G)[Ssp].m(A)}$$$$V2.0 | | |
| SSR-0106208 | RNA1{m(C)[Ssp].m(U)[Ssp].[fl2r](G)[Ssp].m(U)[Ssp]. | CUGUGCUGUA | |
| | [fl2r](G)[Ssp].m(C)[Ssp].[fl2r](U)[n001R].m(G)p.[f | ACACAAGUAG | |
| | l2r](U)p.[fl2r](A)p.[fl2r](A)p.m(C)p.[fl2r](A)p.m( | A | |
| | C)p.[fl2r](A)[n001R].m(A)p.[fl2r](G)p.m(U)p.[fl2r] | | |
| | (A)p.m(G)[Ssp].m(A)}$$$$V2.0 | | |
| SSR-0106209 | RNA1{m(C)[Ssp].m(U)p.[fl2r](G)p.m(U)p.[fl2r](G)p.m | CUGUGCUGUA | |
| | (C)p.[fl2r](U)[n001R].m(G)p.[fl2r](U)p.[fl2r](A)p. | ACACAAGUAG | |
| | [fl2r](A)p.m(C)[Ssp].[fl2r](A)[Ssp].m(C)[Ssp].[fl2 | A | |
| | r](A)[n001R].m(A)[Ssp].[fl2r](G)[Ssp].m(U)[Ssp].[f | | |
| | 12r](A)[Ssp].m(G)[Ssp].m(A)}$$$$V2.0 | | |
| SSR-0106210 | RNA1{m(C)[Ssp].m(U)[Ssp].[fl2r](G)[Ssp].m(U)[Ssp]. | CUGUGCUGUA | |
| | [fl2r](G)[Ssp].m(C)[Ssp].[fl2r](U)[n001R].m(G)[Ssp | ACACAAGUAG | |
| | ].[fl2r](U)[Ssp].[fl2r](A)[Ssp].[fl2r](A)p.m(C)p.[ | A | |
| | fl2r](A)p.m(C)p.[fl2r](A)[n001R].m(A)p.[fl2r](G)p. | | |
| | m(U)p.[fl2r](A)p.m(G)[Ssp].m(A)}$$$$V2.0 | | |
| SSR-0106211 | RNA1{m(C)[Ssp].m(U)[Ssp].[fl2r](G)[Ssp].m(U)[Ssp]. | CUGUGCUGUA | |
| | [fl2r](G)[Ssp].m(C)[Ssp].[fl2r](U)[n001R].m(G)[Ssp | ACACAAGUAG | |
| | ].[fl2r](U)[Ssp].[fl2r](A)[Ssp].[fl2r](A)p.m(C)[Ss | A | |
| | p].[fl2r](A)[Ssp].m(C)[Ssp].[fl2r](A)[n001R].m(A)[ | | |
| | Ssp].[fl2r](G)[Ssp].m(U)[Ssp].[fl2r](A)[Ssp].m(G)[ | | |
| | Ssp].m(A)}$$$$V2.0 | | |
| SSR-0106212 | RNA1{m(C)[Ssp].m(U)[Ssp].[fl2r](G)[Ssp].m(U)[Ssp]. | CUGUGCUGUA | |
| | [fl2r](G)[Ssp].m(C)[Ssp].[fl2r](U)[n001R].m(G)[Ssp | ACACAAGUAG | |
| | ].[fl2r](U)[Ssp].[fl2r](A)[Ssp].[fl2r](A)[Ssp].m(C | A | |
| | )[Ssp].[fl2r](A)[Ssp].m(C)[Ssp].[fl2r](A)[n001R].m | | |
| | (A)[Ssp].[fl2r](G)[Ssp].m(U)[Ssp].[fl2r](A)[Ssp].m | | |
| | (G)[Ssp].m(A)}$$$$V2.0 | | |
| SSR-0106213 | RNA1{m(C)[Ssp].m(U)p.m(G)p.m(U)p.m(G)p.m(C)p.[fl2r | CUGUGCUGUA | |
| | ](U)[n001R].m(G)p.[fl2r](U)p.[fl2r](A)p.[fl2r](A)p | ACACAAGUAG | |
| | .m(C)p.m(A)p.m(C)p.m(A)[n001R].m(A)p.m(G)p.m(U)p.m | A | |
| | (A)p.m(G)[Ssp].m(A)}$$$$V2.0 | | |
| SSR-0106214 | RNA1{m(C)[Ssp].m(U)p.m(G)p.m(U)p.m(G)p.m(C)p.[fl2r | CUGUGCUGUA | |
| | ](U)[n001R].m(G)p.[fl2r](U)p.[fl2r](A)p.[fl2r](A)p | ACACAAGUAG | |
| | .m(C)p.m(A)p.m(C)p.m(A)[n001R].m(A)[Ssp].m(G)[Ssp] | A | |
| | .m(U)[Ssp].m(A)[Ssp].m(G)[Ssp].m(A)}$$$$V2.0 | | |
| SSR-0106215 | RNA1{m(C)[Ssp].m(U)[Ssp].m(G)[Ssp].m(U)[Ssp].m(G)[ | CUGUGCUGUA | |
| | Ssp].m(C)[Ssp].[fl2r](U)[n001R].m(G)p.[fl2r](U)p.[ | ACACAAGUAG | |
| | fl2r](A)p.[fl2r](A)p.m(C)p.m(A)p.m(C)p.m(A)[n001R] | A | |
| | .m(A)p.m(G)p.m(U)p.m(A)p.m(G)[Ssp].m(A)}$$$$V2.0 | | |
| SSR-0106216 | RNA1{m(C)[Ssp].m(U)p.m(G)p.m(U)p.m(G)p.m(C)p.[fl2r | CUGUGCUGUA | |
| | ](U)[n001R].m(G)p.[fl2r](U)p.[fl2r](A)p.[fl2r](A)p | ACACAAGUAG | |
| | .m(C)[Ssp].m(A)[Ssp].m(C)[Ssp].m(A)[n001R].m(A)[Ss | A | |
| | p].m(G)[Ssp].m(U)[Ssp].m(A)[Ssp].m(G)[Ssp].m(A)}$$ | | |
| | $$V2.0 | | |
| SSR-0106217 | RNA1{m(C)[Ssp].m(U)[Ssp].m(G)[Ssp].m(U)[Ssp].m(G)[ | CUGUGCUGUA | |
| | Ssp].m(C)[Ssp].[fl2r](U)[n001R].m(G)[Ssp].[fl2r](U | ACACAAGUAG | |
| | )[Ssp].[fl2r](A)[Ssp].[fl2r](A)p.m(C)p.m(A)p.m(C)p | A | |
| | .m(A)[n001R].m(A)p.m(G)p.m(U)p.m(A)p.m(G)[Ssp].m(A | | |
| | )}$$$$V2.0 | | |
| SSR-0106218 | RNA1{m(C)[Ssp].m(U)[Ssp].m(G)[Ssp].m(U)[Ssp].m(G)[ | CUGUGCUGUA | |
| | Ssp].m(C)[Ssp].[fl2r](U)[n001R].m(G)[Ssp].[fl2r](U | ACACAAGUAG | |
| | )[Ssp].[fl2r](A)[Ssp].[fl2r](A)p.m(C)[Ssp].m(A)[Ss | A | |
| | p].m(C)[Ssp].m(A)[n001R].m(A)[Ssp].m(G)[Ssp].m(U)[ | | |
| | Ssp].m(A)[Ssp].m(G)[Ssp].m(A)}$$$$V2.0 | | |
| SSR-0106219 | RNA1{m(C)[Ssp].m(U)[Ssp].m(G)[Ssp].m(U)[Ssp].m(G)[ | CUGUGCUGUA | |
| | Ssp].m(C)[Ssp].[fl2r](U)[n001R].m(G)[Ssp].[fl2r](U | ACACAAGUAG | |
| | )[Ssp].[fl2r](A)[Ssp].[fl2r](A)[Ssp].m(C)[Ssp].m(A | A | |
| | )[Ssp].m(C)[Ssp].m(A)[n001R].m(A)[Ssp].m(G)[Ssp].m | | |
| | (U)[Ssp].m(A)[Ssp].m(G)[Ssp].m(A)}$$$$V2.0 | | |
| SSR-0106220 | RNA1{p.[Rm5d5m](U)[Ssp].[fl2r](C)[Rsp].m(U)[n001S] | UCUACUUGUG | |
| | .[fl2r](A)p.m(C)p.[fl2r](U)p.m(U)[n001S].[fl2r](G) | UUACAGCACA | |
| | p.m(U)p.[fl2r](G)p.m(U)p.m(U)p.m(A)[Ssp].[fl2r](C) | GUU | |
| | [Ssp].m(A)[Ssp].[fl2r](G)[Ssp].m(C)[Ssp].[fl2r](A) | | |
| | [n001S].m(C)[Ssp].[fl2r](A)[Ssp].m(G)[Ssp].m(U)[Ss | | |
| | p].m(U)}$$$$V2.0 | | |
| SSR-0106221 | RNA1{p.[Rm5d5m](U)[Ssp].[fl2r](C)[Rsp].m(U)[n001S] | UCUACUUGUG | |
| | .[fl2r](A)p.m(C)p.[fl2r](U)p.m(U)[n001S].[fl2r](G) | UUACAGCACA | |
| | p.m(U)p.[fl2r](G)p.m(U)p.m(U)p.m(A)[Ssp].[fl2r](C) | GUU | |
| | [Ssp].m(A)[Ssp].[fl2r](G)[Ssp].m(C)[Ssp].[fl2r](A) | | |
| | [Ssp].m(C)[Ssp].[fl2r](A)[Ssp].m(G)[Ssp].m(U)[Ssp] | | |
| | .m(U)}$$$$V2.0 | | |
| SSR-0106222 | RNA1{p.[Rm5d5m](U)[Rsp].[fl2r](C)[Rsp].m(U)[n001S] | UCUACUUGUG | |
| | .[fl2r](A)p.m(C)p.[fl2r](U)p.m(U)[n001S].[fl2r](G) | UUACAGCACA | |
| | p.m(U)p.[fl2r](G)p.m(U)p.m(U)p.m(A)[Ssp].[fl2r](C) | GUU | |
| | [Ssp].m(A)[Ssp].[fl2r](G)[Ssp].m(C)[Ssp].[fl2r](A) | | |
| | [n001S].m(C)[Ssp].[fl2r](A)[Ssp].m(G)[Ssp].m(U)[Ss | | |
| | p].m(U)}$$$$V2.0 | | |
| SSR-0106223 | RNA1{p.[Rm5d5m](U)[Rsp].[fl2r](C)[Rsp].m(U)[n001S] | UCUACUUGUG | |
| | .[fl2r](A)p.m(C)p.[fl2r](U)p.m(U)[n001S].[fl2r](G) | UUACAGCACA | |
| | p.m(U)p.[fl2r](G)p.m(U)p.m(U)p.m(A)[Ssp].[fl2r](C) | GUU | |
| | [Ssp].m(A)[Ssp].[fl2r](G)[Ssp].m(C)[Ssp].[fl2r](A) | | |
| | [Ssp].m(C)[Ssp].[fl2r](A)[Ssp].m(G)[Ssp].m(U)[Ssp] | | |
| | .m(U)}$$$$V2.0 | | |
| SSR-0106224 | RNA1{p.[Rm5d5m](U)[Ssp].[fl2r](C)[Ssp].m(U)[n001S] | UCUACUUGUG | |
| | .[fl2r](A)p.m(C)p.[fl2r](U)p.m(U)[n001S].[fl2r](G) | UUACAGCACA | |
| | p.m(U)p.[fl2r](G)p.m(U)p.m(U)p.m(A)[Ssp].[fl2r](C) | GUU | |
| | [Ssp].m(A)[Ssp].[fl2r](G)[Ssp].m(C)[Ssp].[fl2r](A) | | |
| | [n001S].m(C)[Ssp].[fl2r](A)[Ssp].m(G)[Ssp].m(U)[Ss | | |
| | p].m(U)}$$$$V2.0 | | |
| SSR-0106225 | RNA1{p.[Rm5d5m](U)[Ssp].[fl2r](C)[Ssp].m(U)[n001S] | UCUACUUGUG | |
| | .[fl2r](A)p.m(C)p.[fl2r](U)p.m(U)[n001S].[fl2r](G) | UUACAGCACA | |
| | p.m(U)p.[fl2r](G)p.m(U)p.m(U)p.m(A)[Ssp].[fl2r](C) | GUU | |
| | [Ssp].m(A)[Ssp].[fl2r](G)[Ssp].m(C)[Ssp].[fl2r](A) | | |
| | [Ssp].m(C)[Ssp].[fl2r](A)[Ssp].m(G)[Ssp].m(U)[Ssp] | | |
| | .m(U)}$$$$V2.0 | | |
| SSR-0106266 | RNA1{m(U)[Ssp].[fl2r](U)[Rsp].m(A)[n001S].[fl2r](U | UUAUAGAGCA | |
| | )p.m(A)p.[fl2r](G)p.m(A)p.m(G)p.m(C)p.m(A)[n001S]. | AGAACACUGU | |
| | [fl2r](A)p.m(G)[n001S].m(A)[n001S].[fl2r](A)p.m(C) | UUU | |
| | p.[fl2r](A)p.m(C)p.m(U)p.m(G)p.m(U)p.m(U)[Ssp].m(U | | |
| | )[Ssp].m(U)}$$$$V2.0 | | |
| SSR-0106267 | RNA1{m(U)[Ssp].[fl2r](U)[Rsp].m(A)[n001S].[fl2r](U | UUAUAGAGCA | |
| | )p.m(A)p.[fl2r](G)p.m(A)p.m(G)p.m(C)p.m(A)[n001S]. | AGAACACUGU | |
| | [fl2r](A)p.m(G)p.m(A)[n001S].[fl2r](A)[n001S].m(C) | UUU | |
| | p.[fl2r](A)p.m(C)p.m(U)p.m(G)p.m(U)p.m(U)[Ssp].m(U | | |
| | )[Ssp].m(U)}$$$$V2.0 | | |
| SSR-0106268 | RNA1{m(U)[Ssp].[fl2r](U)[Rsp].m(A)[n001S].[fl2r](U | UUAUAGAGCA | |
| | )p.m(A)p.[fl2r](G)p.m(A)p.m(G)p.m(C)p.m(A)[n001S]. | AGAACACUGU | |
| | [fl2r](A)p.m(G)p.m(A)p.[fl2r](A)[n001S].m(C)[n001S | UUU | |
| | ].[fl2r](A)p.m(C)p.m(U)p.m(G)p.m(U)p.m(U)[Ssp].m(U | | |
| | )[Ssp].m(U)}$$$$V2.0 | | |
| SSR-0106269 | RNA1{m(U)[Ssp].[fl2r](U)[Rsp].m(A)[n001S].[fl2r](U | UUAUAGAGCA | |
| | )p.m(A)p.[fl2r](G)p.m(A)p.m(G)p.m(C)p.m(A)[n001S]. | AGAACACUGU | |
| | [fl2r](A)p.m(G)p.m(A)p.[fl2r](A)p.m(C)[n001S].[fl2 | UUU | |
| | r](A)[n001S].m(C)p.m(U)p.m(G)p.m(U)p.m(U)[Ssp].m(U | | |
| | )[Ssp].m(U)}$$$$V2.0 | | |
| SSR-0106270 | RNA1{m(U)[Ssp].[fl2r](U)[Rsp].m(A)[n001S].[fl2r](U | UUAUAGAGCA | |
| | )p.m(A)p.[fl2r](G)p.m(A)p.m(G)p.m(C)p.m(A)[n001S]. | AGAACACUGU | |
| | [fl2r](A)p.m(G)p.m(A)p.[fl2r](A)p.m(C)p.[fl2r](A)[ | UUU | |
| | n001S].m(C)[n001S].m(U)p.m(G)p.m(U)p.m(U)[Ssp].m(U | | |
| | )[Ssp].m(U)}$$$$V2.0 | | |
| SSR-0106271 | RNA1{m(U)[Ssp].[fl2r](U)[Rsp].m(A)[n001S].[fl2r](U | UUAUAGAGCA | |
| | )p.m(A)p.[fl2r](G)p.m(A)p.m(G)p.m(C)p.m(A)[n001S]. | AGAACACUGU | |
| | [fl2r](A)p.m(G)p.m(A)p.[fl2r](A)p.m(C)p.[fl2r](A)p | UUU | |
| | .m(C)[n001S].m(U)[n001S].m(G)p.m(U)p.m(U)[Ssp].m(U | | |
| | )[Ssp].m(U)}$$$$V2.0 | | |
| SSR-0106272 | RNA1{m(U)[Ssp].[fl2r](U)[Rsp].m(A)[n001S].[fl2r](U | UUAUAGAGCA | |
| | )p.m(A)p.[fl2r](G)p.m(A)p.m(G)p.m(C)p.m(A)[n001S]. | AGAACACUGU | |
| | [fl2r](A)p.m(G)p.m(A)p.[fl2r](A)p.m(C)p.[fl2r](A)p | UUU | |
| | .m(C)p.m(U)[n001S].m(G)[n001S].m(U)p.m(U)[Ssp].m(U | | |
| | )[Ssp].m(U)}$$$$V2.0 | | |
| SSR-0106273 | RNA1{m(U)[Ssp].[fl2r](U)[Rsp].m(A)[n001S].[fl2r](U | UUAUAGAGCA | |
| | )p.m(A)p.[fl2r](G)p.m(A)p.m(G)p.m(C)p.m(A)[n001S]. | AGAACACUGU | |
| | [fl2r](A)p.m(G)p.m(A)p.[fl2r](A)p.m(C)p.[fl2r](A)p | UUU | |
| | .m(C)p.m(U)p.m(G)[n001S].m(U)[n001S].m(U)[Ssp].m(U | | |
| | )[Ssp].m(U)}$$$$V2.0 | | |
| SSR-0106274 | RNA1{m(U)[Ssp].[fl2r](U)[Rsp].m(A)[n001S].[fl2r](U | UUAUAGAGCA | |
| | )p.m(A)p.[fl2r](G)p.m(A)p.m(G)p.m(C)p.m(A)[n001S]. | AGAACACUGU | |
| | [fl2r](A)p.m(G)[n001S].m(A)[n001S].[fl2r](A)[n001S | UUU | |
| | ].m(C)p.[fl2r](A)p.m(C)p.m(U)p.m(G)p.m(U)p.m(U)[Ss | | |
| | p].m(U)[Ssp].m(U)}$$$$V2.0 | | |
| SSR-0106275 | RNA1{m(U)[Ssp].[fl2r](U)[Rsp].m(A)[n001S].[fl2r](U | UUAUAGAGCA | |
| | )p.m(A)p.[fl2r](G)p.m(A)p.m(G)p.m(C)p.m(A)[n001S]. | AGAACACUGU | |
| | [fl2r](A)p.m(G)p.m(A)[n001S].[fl2r](A)[n001S].m(C) | UUU | |
| | [n001S].[fl2r](A)p.m(C)p.m(U)p.m(G)p.m(U)p.m(U)[Ss | | |
| | p].m(U)[Ssp].m(U)}$$$$V2.0 | | |
| SSR-0106276 | RNA1{m(U)[Ssp].[fl2r](U)[Rsp].m(A)[n001S].[fl2r](U | UUAUAGAGCA | |
| | )p.m(A)p.[fl2r](G)p.m(A)p.m(G)p.m(C)p.m(A)[n001S]. | AGAACACUGU | |
| | [fl2r](A)p.m(G)p.m(A)p.[fl2r](A)[n001S].m(C)[n001S | UUU | |
| | ].[fl2r](A)[n001S].m(C)p.m(U)p.m(G)p.m(U)p.m(U)[Ss | | |
| | p].m(U)[Ssp].m(U)}$$$$V2.0 | | |
| SSR-0106277 | RNA1{m(U)[Ssp].[fl2r](U)[Rsp].m(A)[n001S].[fl2r](U | UUAUAGAGCA | |
| | )p.m(A)p.[fl2r](G)p.m(A)p.m(G)p.m(C)p.m(A)[n001S]. | AGAACACUGU | |
| | [fl2r](A)p.m(G)p.m(A)p.[fl2r](A)p.m(C)[n001S].[fl2 | UUU | |
| | r](A)[n001S].m(C)[n001S].m(U)p.m(G)p.m(U)p.m(U)[Ss | | |
| | p].m(U)[Ssp].m(U)}$$$$V2.0 | | |
| SSR-0106278 | RNA1{m(U)[Ssp].[fl2r](U)[Rsp].m(A)[n001S].[fl2r](U | UUAUAGAGCA | |
| | )p.m(A)p.[fl2r](G)p.m(A)p.m(G)p.m(C)p.m(A)[n001S]. | AGAACACUGU | |
| | [fl2r](A)p.m(G)p.m(A)p.[fl2r](A)p.m(C)p.[fl2r](A)[ | UUU | |
| | n001S].m(C)[n001S].m(U)[n001S].m(G)p.m(U)p.m(U)[Ss | | |
| | p].m(U)[Ssp].m(U)}$$$$V2.0 | | |
| SSR-0106279 | RNA1{m(U)[Ssp].[fl2r](U)[Rsp].m(A)[n001S].[fl2r](U | UUAUAGAGCA | |
| | )p.m(A)p.[fl2r](G)p.m(A)p.m(G)p.m(C)p.m(A)[n001S]. | AGAACACUGU | |
| | [fl2r](A)p.m(G)p.m(A)p.[fl2r](A)p.m(C)p.[fl2r](A)p | UUU | |
| | .m(C)[n001S].m(U)[n001S].m(G)[n001S].m(U)p.m(U)[Ss | | |
| | p].m(U)[Ssp].m(U)}$$$$V2.0 | | |
| SSR-0106280 | RNA1{m(U)[Ssp].[fl2r](U)[Rsp].m(A)[n001S].[fl2r](U | UUAUAGAGCA | |
| | )p.m(A)p.[fl2r](G)p.m(A)p.m(G)p.m(C)p.m(A)[n001S]. | AGAACACUGU | |
| | [fl2r](A)p.m(G)p.m(A)p.[fl2r](A)p.m(C)p.[fl2r](A)p | UUU | |
| | .m(C)p.m(U)[n001S].m(G)[n001S].m(U)[n001S].m(U)[Ss | | |
| | p].m(U)[Ssp].m(U)}$$$$V2.0 | | |
| SSR-0106281 | RNA1{m(U)[Ssp].[fl2r](U)[Rsp].m(A)[n001S].[fl2r](U | UUAUAGAGCA | |
| | )p.m(A)p.[fl2r](G)p.m(A)p.m(G)p.m(C)p.m(A)[n001S]. | AGAACACUGU | |
| | [fl2r](A)p.m(G)[n001S].m(A)[n001S].[fl2r](A)[n001S | UUU | |
| | ].m(C)[n001S].[fl2r](A)p.m(C)p.m(U)p.m(G)p.m(U)p.m | | |
| | (U)[Ssp].m(U)[Ssp].m(U)}$$$$V2.0 | | |
| SSR-0106282 | RNA1{m(U)[Ssp].[fl2r](U)[Rsp].m(A)[n001S].[fl2r](U | UUAUAGAGCA | |
| | )p.m(A)p.[fl2r](G)p.m(A)p.m(G)p.m(C)p.m(A)[n001S]. | AGAACACUGU | |
| | [fl2r](A)p.m(G)p.m(A)[n001S].[fl2r](A)[n001S].m(C) | UUU | |
| | [n001S].[fl2r](A)[n001S].m(C)p.m(U)p.m(G)p.m(U)p.m | | |
| | (U)[Ssp].m(U)[Ssp].m(U)}$$$$V2.0 | | |
| SSR-0106283 | RNA1{m(U)[Ssp].[fl2r](U)[Rsp].m(A)[n001S].[fl2r](U | UUAUAGAGCA | |
| | )p.m(A)p.[fl2r](G)p.m(A)p.m(G)p.m(C)p.m(A)[n001S]. | AGAACACUGU | |
| | [fl2r](A)p.m(G)p.m(A)p.[fl2r](A)[n001S].m(C)[n001S | UUU | |
| | ].[fl2r](A)[n001S].m(C)[n001S].m(U)p.m(G)p.m(U)p.m | | |
| | (U)[Ssp].m(U)[Ssp].m(U)}$$$$V2.0 | | |
| SSR-0106284 | RNA1{m(U)[Ssp].[fl2r](U)[Rsp].m(A)[n001S].[fl2r](U | UUAUAGAGCA | |
| | )p.m(A)p.[fl2r](G)p.m(A)p.m(G)p.m(C)p.m(A)[n001S]. | AGAACACUGU | |
| | [fl2r](A)p.m(G)p.m(A)p.[fl2r](A)p.m(C)[n001S].[fl2 | UUU | |
| | r](A)[n001S].m(C)[n001S].m(U)[n001S].m(G)p.m(U)p.m | | |
| | (U)[Ssp].m(U)[Ssp].m(U)}$$$$V2.0 | | |
| SSR-0106285 | RNA1{m(U)[Ssp].[fl2r](U)[Rsp].m(A)[n001S].[fl2r](U | UUAUAGAGCA | |
| | )p.m(A)p.[fl2r](G)p.m(A)p.m(G)p.m(C)p.m(A)[n001S]. | AGAACACUGU | |
| | [fl2r](A)p.m(G)p.m(A)p.[fl2r](A)p.m(C)p.[fl2r](A)[ | UUU | |
| | n001S].m(C)[n001S].m(U)[n001S].m(G)[n001S].m(U)p.m | | |
| | (U)[Ssp].m(U)[Ssp].m(U)}$$$$V2.0 | | |
| SSR-0106286 | RNA1{m(U)[Ssp].[fl2r](U)[Rsp].m(A)[n001S].[fl2r](U | UUAUAGAGCA | |
| | )p.m(A)p.[fl2r](G)p.m(A)p.m(G)p.m(C)p.m(A)[n001S]. | AGAACACUGU | |
| | [fl2r](A)p.m(G)p.m(A)p.[fl2r](A)p.m(C)p.[fl2r](A)p | UUU | |
| | .m(C)[n001S].m(U)[n001S].m(G)[n001S].m(U)[n001S].m | | |
| | (U)[Ssp].m(U)[Ssp].m(U)}$$$$V2.0 | | |
| SSR-0106287 | RNA1{m(U)[Ssp].[fl2r](U)[Rsp].m(A)[n001S].[fl2r](U | UUAUAGAGCA | |
| | )p.m(A)p.[fl2r](G)p.m(A)p.m(G)p.m(C)p.m(A)[n001S]. | AGAACACUGU | |
| | [fl2r](A)p.m(G)[n001S].m(A)p.[fl2r](A)[n001S].m(C) | UUU | |
| | p.[fl2r](A)[n001S].m(C)p.m(U)[n001S].m(G)p.m(U)[n0 | | |
| | 01S].m(U)[Ssp].m(U)[Ssp].m(U)}$$$$V2.0 | | |
| SSR-0106288 | RNA1{m(U)[Ssp].[fl2r](U)[Rsp].m(A)[n001S].[fl2r](U | UUAUAGAGCA | |
| | )p.m(A)p.[fl2r](G)p.m(A)p.m(G)p.m(C)p.m(A)[n001S]. | AGAACACUGU | |
| | [fl2r](A)p.m(G)p.m(A)[n001S].[fl2r](A)p.m(C)[n001S | UUU | |
| | ].[fl2r](A)p.m(C)[n001S].m(U)p.m(G)[n001S].m(U)p.m | | |
| | (U)[Ssp].m(U)[Ssp].m(U)}$$$$V2.0 | | |
| SSR-0106289 | RNA1{m(U)[Ssp].[fl2r](U)[Rsp].m(A)[n001S].[fl2r](U | UUAUAGAGCA | |
| | )p.m(A)p.[fl2r](G)p.m(A)p.m(G)p.m(C)p.m(A)[n001S]. | AGAACACUGU | |
| | [fl2r](A)p.m(G)[n001S].[fl2r](A)p.[fl2r](A)[n001S] | UUU | |
| | .[fl2r](C)p.[fl2r](A)[n001S].[fl2r](C)p.m(U)[n001S | | |
| | ].[fl2r](G)p.m(U)[n001S].[fl2r](U)[Ssp].m(U)[Ssp]. | | |
| | m(U)}$$$$V2.0 | | |
| SSR-0106290 | RNA1{m(U)[Ssp].[fl2r](U)[Rsp].m(A)[n001S].[fl2r](U | UUAUAGAGCA | |
| | )p.m(A)p.[fl2r](G)p.m(A)p.m(G)p.m(C)p.m(A)[n001S]. | AGAACACUGU | |
| | [fl2r](A)p.m(G)p.m(A)[n001S].[fl2r](A)p.m(C)[n001S | UUU | |
| | ].[fl2r](A)p.m(C)[n001S].[fl2r](U)p.m(G)[n001S].[f | | |
| | l2r](U)p.m(U)[Ssp].m(U)[Ssp].m(U)}$$$$V2.0 | | |
| SSR-0106291 | RNA1{m(U)[Ssp].[fl2r](U)[Rsp].m(A)[n001S].[fl2r](U | UUAUAGAGCA | |
| | )p.m(A)p.[fl2r](G)p.m(A)p.m(G)p.m(C)p.m(A)[n001S]. | AGAACACUGU | |
| | [fl2r](A)p.m(G)p.m(A)[n001S].[fl2r](A)[n001S].m(C) | UUU | |
| | p.[fl2r](A)p.m(C)[n001S].m(U)[n001S].m(G)p.m(U)p.m | | |
| | (U)[Ssp].m(U)[Ssp].m(U)}$$$$V2.0 | | |
| SSR-0106292 | RNA1{m(U)[Ssp].[fl2r](U)[Rsp].m(A)[n001S].[fl2r](U | UUAUAGAGCA | |
| | )p.m(A)p.[fl2r](G)p.m(A)p.m(G)p.m(C)p.m(A)[n001S]. | AGAACACUGU | |
| | [fl2r](A)p.m(G)p.m(A)p.[fl2r](A)[n001S].m(C)[n001S | UUU | |
| | ].[fl2r](A)p.m(C)p.m(U)[n001S].m(G)[n001S].m(U)p.m | | |
| | (U)[Ssp].m(U)[Ssp].m(U)}$$$$V2.0 | | |
| SSR-0106293 | RNA1{m(U)[Ssp].[fl2r](U)[Rsp].m(A)[n001S].[fl2r](U | UUAUAGAGCA | |
| | )p.m(A)p.[fl2r](G)p.m(A)p.m(G)p.m(C)p.m(A)[n001S]. | AGAACACUGU | |
| | [fl2r](A)p.m(G)[n001S].m(A)p.[fl2r](A)p.m(C)p.[fl2 | UUU | |
| | r](A)p.m(C)p.m(U)p.m(G)p.m(U)p.m(U)[Ssp].m(U)[Ssp] | | |
| | .m(U)}$$$$V2.0 | | |
| SSR-0106294 | RNA1{m(U)[Ssp].[fl2r](U)[Rsp].m(A)[n001S].[fl2r](U | UUAUAGAGCA | |
| | )p.m(A)p.[fl2r](G)p.m(A)p.m(G)p.m(C)p.m(A)[n001S]. | AGAACACUGU | |
| | [fl2r](A)p.m(G)p.m(A)[n001S].[fl2r](A)p.m(C)p.[fl2 | UUU | |
| | r](A)p.m(C)p.m(U)p.m(G)p.m(U)p.m(U)[Ssp].m(U)[Ssp] | | |
| | .m(U)}$$$$V2.0 | | |
| SSR-0106295 | RNA1{m(U)[Ssp].[fl2r](U)[Rsp].m(A)[n001S].[fl2r](U | UUAUAGAGCA | |
| | )p.m(A)p.[fl2r](G)p.m(A)p.m(G)p.m(C)p.m(A)[n001S]. | AGAACACUGU | |
| | [fl2r](A)p.m(G)p.m(A)p.[fl2r](A)[n001S].m(C)p.[fl2 | UUU | |
| | r](A)p.m(C)p.m(U)p.m(G)p.m(U)p.m(U)[Ssp].m(U)[Ssp] | | |
| | .m(U)}$$$$V2.0 | | |
| SSR-0106296 | RNA1{m(U)[Ssp].[fl2r](U)[Rsp].m(A)[n001S].[fl2r](U | UUAUAGAGCA | |
| | )p.m(A)p.[fl2r](G)p.m(A)p.m(G)p.m(C)p.m(A)[n001S]. | AGAACACUGU | |
| | [fl2r](A)p.m(G)p.m(A)p.[fl2r](A)p.m(C)[n001S].[fl2 | UUU | |
| | r](A)p.m(C)p.m(U)p.m(G)p.m(U)p.m(U)[Ssp].m(U)[Ssp] | | |
| | .m(U)}$$$$V2.0 | | |
| SSR-0106297 | RNA1{m(U)[Ssp].[fl2r](U)[Rsp].m(A)[n001S].[fl2r](U | UUAUAGAGCA | |
| | )p.m(A)p.[fl2r](G)p.m(A)p.m(G)p.m(C)p.m(A)[n001S]. | AGAACACUGU | |
| | [fl2r](A)p.m(G)p.m(A)p.[fl2r](A)p.m(C)p.[fl2r](A)[ | UUU | |
| | n001S].m(C)p.m(U)p.m(G)p.m(U)p.m(U)[Ssp].m(U)[Ssp] | | |
| | .m(U)}$$$$V2.0 | | |
| SSR-0106298 | RNA1{m(U)[Ssp].[fl2r](U)[Rsp].m(A)[n001S].[fl2r](U | UUAUAGAGCA | |
| | )p.m(A)p.[fl2r](G)p.m(A)p.m(G)p.m(C)p.m(A)[n001S]. | AGAACACUGU | |
| | [fl2r](A)p.m(G)p.m(A)p.[fl2r](A)p.m(C)p.[fl2r](A)p | UUU | |
| | .m(C)[n001S].m(U)p.m(G)p.m(U)p.m(U)[Ssp].m(U)[Ssp] | | |
| | .m(U)}$$$$V2.0 | | |
| SSR-0106299 | RNA1{m(U)[Ssp].[fl2r](U)[Rsp].m(A)[n001S].[fl2r](U | UUAUAGAGCA | |
| | )p.m(A)p.[fl2r](G)p.m(A)p.m(G)p.m(C)p.m(A)[n001S]. | AGAACACUGU | |
| | [fl2r](A)p.m(G)p.m(A)p.[fl2r](A)p.m(C)p.[fl2r](A)p | UUU | |
| | .m(C)p.m(U)[n001S].m(G)p.m(U)p.m(U)[Ssp].m(U)[Ssp] | | |
| | .m(U)}$$$$V2.0 | | |
| SSR-0106300 | RNA1{m(U)[Ssp].[fl2r](U)[Rsp].m(A)[n001S].[fl2r](U | UUAUAGAGCA | |
| | )p.m(A)p.[fl2r](G)p.m(A)p.m(G)p.m(C)p.m(A)[n001S]. | AGAACACUGU | |
| | [fl2r](A)p.m(G)p.m(A)p.[fl2r](A)p.m(C)p.[fl2r](A)p | UUU | |
| | .m(C)p.m(U)p.m(G)[n001S].m(U)p.m(U)[Ssp].m(U)[Ssp] | | |
| | .m(U)}$$$$V2.0 | | |
| SSR-0106301 | RNA1{m(U)[Ssp].[fl2r](U)[Rsp].m(A)[n001S].[fl2r](U | UUAUAGAGCA | |
| | )p.m(A)p.[fl2r](G)p.m(A)p.m(G)p.m(C)p.m(A)[n001S]. | AGAACACUGU | |
| | [fl2r](A)p.m(G)p.m(A)p.[fl2r](A)p.m(C)p.[fl2r](A)p | UUU | |
| | .m(C)p.m(U)p.m(G)p.m(U)[n001S].m(U)[Ssp].m(U)[Ssp] | | |
| | .m(U)}$$$$V2.0 | | |
| SSR-0106302 | RNA1{m(U)[Ssp].[fl2r](U)[Rsp].m(A)[n001S].[fl2r](U | UUAUAGAGCA | |
| | )p.m(A)p.[fl2r](G)p.m(A)p.m(G)p.m(C)p.m(A)[n001S]. | AGAACACUGU | |
| | [fl2r](A)p.m(G)[n001S].[fl2r](A)p.[fl2r](A)p.m(C)p | UUU | |
| | .[fl2r](A)p.m(C)p.m(U)p.m(G)p.m(U)p.m(U)[Ssp].m(U) | | |
| | [Ssp].m(U)}$$$$V2.0 | | |
| SSR-0106303 | RNA1{m(U)[Ssp].[fl2r](U)[Rsp].m(A)[n001S].[fl2r](U | UUAUAGAGCA | |
| | )p.m(A)p.[fl2r](G)p.m(A)p.m(G)p.m(C)p.m(A)[n001S]. | AGAACACUGU | |
| | [fl2r](A)p.m(G)p.m(A)p.[fl2r](A)[n001S].[fl2r](C)p | UUU | |
| | .[fl2r](A)p.m(C)p.m(U)p.m(G)p.m(U)p.m(U)[Ssp].m(U) | | |
| | [Ssp].m(U)}$$$$V2.0 | | |
| SSR-0106304 | RNA1{m(U)[Ssp].[fl2r](U)[Rsp].m(A)[n001S].[fl2r](U | UUAUAGAGCA | |
| | )p.m(A)p.[fl2r](G)p.m(A)p.m(G)p.m(C)p.m(A)[n001S]. | AGAACACUGU | |
| | [fl2r](A)p.m(G)p.m(A)p.[fl2r](A)p.m(C)p.[fl2r](A)[ | UUU | |
| | n001S].[fl2r](C)p.m(U)p.m(G)p.m(U)p.m(U)[Ssp].m(U) | | |
| | [Ssp].m(U)}$$$$V2.0 | | |
| SSR-0106305 | RNA1{m(U)[Ssp].[fl2r](U)[Rsp].m(A)[n001S].[fl2r](U | UUAUAGAGCA | |
| | )p.m(A)p.[fl2r](G)p.m(A)p.m(G)p.m(C)p.m(A)[n001S]. | AGAACACUGU | |
| | [fl2r](A)p.m(G)p.m(A)p.[fl2r](A)p.m(C)p.[fl2r](A)p | UUU | |
| | .m(C)[n001S].[fl2r](U)p.m(G)p.m(U)p.m(U)[Ssp].m(U) | | |
| | [Ssp].m(U)}$$$$V2.0 | | |
| SSR-0106306 | RNA1{m(U)[Ssp].[fl2r](U)[Rsp].m(A)[n001S].[fl2r](U | UUAUAGAGCA | |
| | )p.m(A)p.[fl2r](G)p.m(A)p.m(G)p.m(C)p.m(A)[n001S]. | AGAACACUGU | |
| | [fl2r](A)p.m(G)p.m(A)p.[fl2r](A)p.m(C)p.[fl2r](A)p | UUU | |
| | .m(C)p.m(U)[n001S].[fl2r](G)p.m(U)p.m(U)[Ssp].m(U) | | |
| | [Ssp].m(U)}$$$$V2.0 | | |
| SSR-0106307 | RNA1{m(U)[Ssp].[fl2r](U)[Rsp].m(A)[n001S].[fl2r](U | UUAUAGAGCA | |
| | )p.m(A)p.[fl2r](G)p.m(A)p.m(G)p.m(C)p.m(A)[n001S]. | AGAACACUGU | |
| | [fl2r](A)p.m(G)p.m(A)p.[fl2r](A)p.m(C)p.[fl2r](A)p | UUU | |
| | .m(C)p.m(U)p.m(G)p.m(U)[n001S].[fl2r](U)[Ssp].m(U) | | |
| | [Ssp].m(U)}$$$$V2.0 | | |
| SSR-0106445 | RNA1{m(C)[sp].m(U)p.[fl2r](G)p.m(U)p.[fl2r](G)p.m( | CUGUGCUGUA | |
| | C)p.[fl2r](U)[n009R].m(G)p.[fl2r](U)p.[fl2r](A)p.[ | ACACAAGUAG | |
| | fl2r](A)p.m(C)p.[fl2r](A)p.m(C)p.[fl2r](A)p.m(A)p. | A | |
| | [fl2r](G)p.m(U)p.[fl2r](A)p.m(G)[sp].[fl2r](A)}$$$ | | |
| | $V2.0 | | |
| SSR-0106446 | RNA1{m(C)[sp].m(U)p.[fl2r](G)p.m(U)p.[fl2r](G)p.m( | CUGUGCUGUA | |
| | C)p.[fl2r](U)[n033R].m(G)p.[fl2r](U)p.[fl2r](A)p.[ | ACACAAGUAG | |
| | fl2r](A)p.m(C)p.[fl2r](A)p.m(C)p.[fl2r](A)p.m(A)p. | A | |
| | [fl2r](G)p.m(U)p.[fl2r](A)p.m(G)[sp].[fl2r](A)}$$$ | | |
| | $V2.0 | | |
| SSR-0106447 | RNA1{m(C)[Ssp].m(U)p.[fl2r](G)p.m(U)p.[fl2r](G)p.m | CUGUGCUGUA | |
| | (C)p.[fl2r](U)[n009R].m(G)p.[fl2r](U)p.[fl2r](A)p. | ACACAAGUAG | |
| | [fl2r](A)p.m(C)p.[fl2r](A)p.m(C)p.[fl2r](A)p.m(A)p | A | |
| | .[fl2r](G)p.m(U)p.[fl2r](A)p.m(G)[Ssp].[fl2r](A)}$ | | |
| | $$$V2.0 | | |
| SSR-0106448 | RNA1{m(C)[Ssp].m(U)p.[fl2r](G)p.m(U)p.[fl2r](G)p.m | CUGUGCUGUA | |
| | (C)p.[fl2r](U)[n033R].m(G)p.[fl2r](U)p.[fl2r](A)p. | ACACAAGUAG | |
| | [fl2r](A)p.m(C)p.[fl2r](A)p.m(C)p.[fl2r](A)p.m(A)p | A | |
| | .[fl2r](G)p.m(U)p.[fl2r](A)p.m(G)[Ssp].[fl2r](A)}$ | | |
| | $$$V2.0 | | |
| SSR-0106449 | RNA1{m(C)[Ssp].m(U)p.[fl2r](G)p.m(U)p.[fl2r](G)p.m | CUGUGCUGUA | |
| | (C)p.[fl2r](U)[n009R].m(G)p.[fl2r](U)p.[fl2r](A)p. | ACACAAGUAG | |
| | [fl2r](A)p.m(C)p.[fl2r](A)p.m(C)p.[fl2r](A)[n001R] | A | |
| | .m(A)p.[fl2r](G)p.m(U)p.[fl2r](A)p.m(G)[Ssp].[fl2r | | |
| | ](A)}$$$$V2.0 | | |
| SSR-0106450 | RNA1{m(C)[Ssp].m(U)p.[fl2r](G)p.m(U)p.[fl2r](G)p.m | CUGUGCUGUA | |
| | (C)p.[fl2r](U)[n033R].m(G)p.[fl2r](U)p.[fl2r](A)p. | ACACAAGUAG | |
| | [fl2r](A)p.m(C)p.[fl2r](A)p.m(C)p.[fl2r](A)[n001R] | A | |
| | .m(A)p.[fl2r](G)p.m(U)p.[fl2r](A)p.m(G)[Ssp].[fl2r | | |
| | ](A)}$$$$V2.0 | | |
| SSR-0106451 | RNA1{m(C)[Ssp].m(U)[Ssp].[fl2r](G)[Ssp].m(U)[Ssp]. | CUGUGCUGUA | |
| | [fl2r](G)[Ssp].m(C)[Ssp].[fl2r](U)[n009R].m(G)p.[f | ACACAAGUAG | |
| | l2r](U)p.[fl2r](A)p.[fl2r](A)p.m(C)p.[fl2r](A)p.m( | A | |
| | C)p.[fl2r](A)[n001R].m(A)[Ssp].[fl2r](G)[Ssp].m(U) | | |
| | [Ssp].[fl2r](A)[Ssp].m(G)[Ssp].m(A)}$$$$V2.0 | | |
| SSR-0106452 | RNA1{m(C)[Ssp].m(U)[Ssp].[fl2r](G)[Ssp].m(U)[Ssp]. | CUGUGCUGUA | |
| | [fl2r](G)[Ssp].m(C)[Ssp].[fl2r](U)[n033R].m(G)p.[f | ACACAAGUAG | |
| | l2r](U)p.[fl2r](A)p.[fl2r](A)p.m(C)p.[fl2r](A)p.m( | A | |
| | C)p.[fl2r](A)[n001R].m(A)[Ssp].[fl2r](G)[Ssp].m(U) | | |
| | [Ssp].[fl2r](A)[Ssp].m(G)[Ssp].m(A)}$$$$V2.0 | | |
| SSR-0106479 | RNA1{p.[Rm5d5m](U)[Rsp].[fl2r](C)[Ssp].m(U)[n065S] | UCUACUUGUG | |
| | .[fl2r](A)p.m(C)p.[fl2r](U)p.m(U)[n065S].[fl2r](G) | UUACAGCACA | |
| | p.m(U)p.[fl2r](G)p.m(U)p.m(U)p.m(A)[Ssp].[fl2r](C) | GUU | |
| | [Ssp].m(A)[Ssp].[fl2r](G)[Ssp].m(C)[Ssp].[fl2r](A) | | |
| | [n065S].m(C)[Ssp].[fl2r](A)[Ssp].m(G)[Ssp].m(U)[Ss | | |
| | p].m(U)}$$$$V2.0 | | |
| SSR-0106480 | RNA1{p.[Rm5d5m](U)[Rsp].[fl2r](C)[Ssp].m(U)[n070S] | UCUACUUGUG | |
| | .[fl2r](A)p.m(C)p.[fl2r](U)p.m(U)[n070S].[fl2r](G) | UUACAGCACA | |
| | p.m(U)p.[fl2r](G)p.m(U)p.m(U)p.m(A)[Ssp].[fl2r](C) | GUU | |
| | [Ssp].m(A)[Ssp].[fl2r](G)[Ssp].m(C)[Ssp].[fl2r](A) | | |
| | [n070S].m(C)[Ssp].[fl2r](A)[Ssp].m(G)[Ssp].m(U)[Ss | | |
| | p].m(U)}$$$$V2.0 | | |
| SSR-0106481 | RNA1{p.[Rm5d5m](U)[Rsp].[fl2r](C)[Ssp].m(U)[n071S] | UCUACUUGUG | |
| | .[fl2r](A)p.m(C)p.[fl2r](U)p.m(U)[n071S].[fl2r](G) | UUACAGCACA | |
| | p.m(U)p.[fl2r](G)p.m(U)p.m(U)p.m(A)[Ssp].[fl2r](C) | GUU | |
| | [Ssp].m(A)[Ssp].[fl2r](G)[Ssp].m(C)[Ssp].[fl2r](A) | | |
| | [n071S].m(C)[Ssp].[fl2r](A)[Ssp].m(G)[Ssp].m(U)[Ss | | |
| | p].m(U)}$$$$V2.0 | | |
| SSR-0106482 | RNA1{m(U)[Rsp].[fl2r](C)[Ssp].m(U)[n065S].[fl2r](A | UCUACUUGUG | |
| | )p.m(C)p.[fl2r](U)p.m(U)[n065S].[fl2r](G)p.m(U)p.[ | UUACAGCACA | |
| | fl2r](G)p.m(U)p.m(U)p.m(A)[Ssp].[fl2r](C)[Ssp].m(A | GUU | |
| | )[Ssp].[fl2r](G)[Ssp].m(C)[Ssp].[fl2r](A)[n065S].m | | |
| | (C)[Ssp].[fl2r](A)[Ssp].m(G)[Ssp].m(U)[Ssp].m(U)}$ | | |
| | $$$V2.0 | | |
| SSR-0106483 | RNA1{m(U)[Rsp].[fl2r](C)[Ssp].m(U)[n070S].[fl2r](A | UCUACUUGUG | |
| | )p.m(C)p.[fl2r](U)p.m(U)[n070S].[fl2r](G)p.m(U)p.[ | UUACAGCACA | |
| | fl2r](G)p.m(U)p.m(U)p.m(A)[Ssp].[fl2r](C)[Ssp].m(A | GUU | |
| | )[Ssp].[fl2r](G)[Ssp].m(C)[Ssp].[fl2r](A)[n070S].m | | |
| | (C)[Ssp].[fl2r](A)[Ssp].m(G)[Ssp].m(U)[Ssp].m(U)}$ | | |
| | $$$V2.0 | | |
| SSR-0106484 | RNA1{m(U)[Rsp].[fl2r](C)[Ssp].m(U)[n071S].[fl2r](A | UCUACUUGUG | |
| | )p.m(C)p.[fl2r](U)p.m(U)[n071S].[fl2r](G)p.m(U)p.[ | UUACAGCACA | |
| | fl2r](G)p.m(U)p.m(U)p.m(A)[Ssp].[fl2r](C)[Ssp].m(A | GUU | |
| | )[Ssp].[fl2r](G)[Ssp].m(C)[Ssp].[fl2r](A)[n071S].m | | |
| | (C)[Ssp].[fl2r](A)[Ssp].m(G)[Ssp].m(U)[Ssp].m(U)}$ | | |
| | $$$V2.0 | | |
| SSR-0106487 | RNA1{m(C)[Ssp].m(U)p.[fl2r](G)p.m(U)p.[fl2r](G)p.m | CUGUGCUGUA | |
| | (C)p.[fl2r](U)[n069R].m(G)p.[fl2r](U)p.[fl2r](A)p. | ACACAAGUAG | |
| | [fl2r](A)p.m(C)p.[fl2r](A)p.m(C)p.[fl2r](A)[n069R] | A | |
| | .m(A)p.[fl2r](G)p.m(U)p.[fl2r](A)p.m(G)[Ssp].[fl2r | | |
| | ](A)}$$$$V2.0 | | |
| SSR-0106517 | RNA1{[vped5m](U)[Ssp].[fl2r](C)[Ssp].m(U)[n001S].[ | UCUACUUGUG | |
| | fl2r](A)p.m(C)p.[fl2r](U)p.m(U)[n001S].[fl2r](G)p. | UUACAGCACA | |
| | m(U)p.[fl2r](G)p.m(U)p.m(U)p.m(A)[Ssp].[fl2r](C)[S | GUU | |
| | sp].m(A)[Ssp].[fl2r](G)[Ssp].m(C)[Ssp].[fl2r](A)[n | | |
| | 001S].m(C)[Ssp].[fl2r](A)[Ssp].m(G)[Ssp].m(U)[Ssp] | | |
| | .m(U)}$$$$V2.0 | | |
| SSR-0106564 | RNA1{p.m(C)[Ssp].m(U)p.[fl2r](G)p.m(U)p.[f]2r](G)p | CUGUGCUGUA | |
| | .m(C)p.[fl2r](U)[n001R].m(G)p.[fl2r](U)p.[fl2r](A) | ACACAAGUAG | |
| | p.[fl2r](A)p.m(C)p.[fl2r](A)p.m(C)p.[fl2r](A)[n001 | A | |
| | R].m(A)p.[f]2r](G)p.m(U)p.[fl2r](A)p.m(G)[Ssp].[fl | | |
| | 2r](A)}\|CHEM1{[nC6o]$CHEM1,RNA1,1:R1-1:R1$$$V2.0 | | |
| SSR-0106565 | A)p.[fl2r](A)p.m(C)p.[fl2r](A)p.m(C)p.[f]2r](A)[n0 | CUGUGCUGUA | |
| | 01R].m(A)p.[fl2r](G)p.m(U)p.[fl2r](A)p.m(G)[Ssp].[ | ACACAAGUAG | |
| | fl2r](A)}\|CHEM1{[nC6o]}\|CHEM2{[C16oyl]}$CHEM1,RNA1 | A | |
| | ,1:R1-1:R1\|CHEM2,CHEM1,1:R1-1:R2$$$V2.0 | | |
| SSR-0106566 | RNA1{p.m(C)[Ssp].m(U)p.[fl2r](G)p.m(U)p.[f]2r](G)p | CUGUGCUGUA | |
| | .m(C)p.[f12r](U)[n001R].m(G)p.[fl2r](U)p.[f12r](A) | ACACAAGUAG | |
| | p.[fl2r](A)p.m(C)p.[f12r](A)p.m(C)p.[f]2r](A)[n001 | A | |
| | R].m(A)p.[f]2r](G)p.m(U)p.[fl2r](A)p.m(G)[Ssp].[fl | | |
| | 2r](A)}\|CHEM1{[nC6o]}\|CHEM2{[C18oyl]}$CHEM1,RNA1,1 | | |
| | :R1-1:R1\|CHEM2,CHEM1,1:R1-1:R2$$$V2.0 | | |
| SSR-0106568 | RNA1{p.m(C)[Ssp].m(U)p.[fl2r](G)p.m(U)p.[f]2r](G)p | CUGUGCUGUA | |
| | .m(C)p.[f12r](U)[n001R].m(G)p.[fl2r](U)p.[f12r](A) | ACACAAGUAG | |
| | p.[fl2r](A)p.m(C)p.[fl2r](A)p.m(C)p.[f]2r](A)[n001 | A | |
| | R].m(A)p.[f]2r](G)p.m(U)p.[fl2r](A)p.m(G)[Ssp].[fl | | |
| | 2r](A)}\|CHEM1{[nC6o]}\|CHEM2{[lin]}$CHEM1,RNA1,1:R1 | | |
| | -1:R1\|CHEM2,CHEM1,1:R1-1:R2$$$V2.0 | | |
| SSR-0106569 | RNA1{p.m(C)[Ssp].m(U)p.[fl2r](G)p.m(U)p.[fl2r](G)p | CUGUGCUGUA | |
| | .m(C)p.[f12r](U)[n001R].m(G)p.[f12r](U)p.[f]2r](A) | ACACAAGUAG | |
| | p.[fl2r](A)p.m(C)p.[fl2r](A)p.m(C)p.[f]2r](A)[n001 | A | |
| | R].m(A)p.[f]2r](G)p.m(U)p.[fl2r](A)p.m(G)[Ssp].[fl | | |
| | 2r](A)}\|CHEM1{[nC6o]}\|CHEM2{[Srl1C11oyl]}$CHEM1,RN | | |
| | A1,1:R1-1:R1\|CHEM2,CHEM1,1:R1-1:R2$$$V2.0 | | |
| SSR-0106580 | RNA1{p.m(C)[Ssp].m(U)p.[fl2r](G)p.m(U)p.[fl2r](G)p | CUGUGCUGUA | |
| | .m(C)p.[fl2r](U)[n001R].m(G)p.[fl2r](U)p.[fl2r](A) | ACACAAGUAG | |
| | p.[fl2r](A)p.m(C)p.[fl2r](A)p.m(C)p.[fl2r](A)[n001 | A | |
| | R].m(A)p.[fl2r](G)p.m(U)p.[fl2r](A)p.m(G)[Ssp].[fl | | |
| | 2r](A)}\|CHEM1{[nC6o]}\|CHEM2{[Citr]}$CHEM1,RNA1,1:R | | |
| | 1-1:R1\|CHEM2,CHEM1,1:R1-1:R2$$$V2.0 | | |
| SSR-0106652 | RNA1{p.m(C)[Ssp].m(U)p.[fl2r](G)p.m(U)p.[fl2r](G)p | CUGUGCUGUA | |
| | .m(C)p.[fl2r](U)[n001R].m(G)p.[fl2r](U)p.[fl2r](A) | ACACAAGUAG | |
| | p.[fl2r](A)p.m(C)p.[fl2r](A)p.m(C)p.[fl2r](A)[n001 | A | |
| | R].m(A)p.[fl2r](G)p.m(U)p.[fl2r](A)p.m(G)[Ssp].[fl | | |
| | 2r](A)}\|CHEM1{[nC6o]}\|CHEM2{[Phodec]}$CHEM1,RNA1,1 | | |
| | :R1-1:R1\|CHEM2,CHEM1,1:R1-1:R2$$$V2.0 | | |
| SSR-0106653 | RNA1{p.m(C)[Ssp].m(U)p.[fl2r](G)p.m(U)p.[fl2r](G)p | CUGUGCUGUA | |
| | .m(C)p.[fl2r](U)[n001R].m(G)p.[fl2r](U)p.[fl2r](A) | ACACAAGUAG | |
| | p.[fl2r](A)p.m(C)p.[fl2r](A)p.m(C)p.[fl2r](A)[n001 | A | |
| | R].m(A)p.[fl2r](G)p.m(U)p.[fl2r](A)p.m(G)[Ssp].[fl | | |
| | 2r](A)}\|CHEM1{[nC6o]}\|CHEM2{[EPhodec]}$CHEM1,RNA1, | | |
| | 1:R1-1:R1\|CHEM2,CHEM1,1:R1-1:R2$$$V2.0 | | |
| SSR-0106660 | RNA1{p.m(C)[Ssp].m(U)p.[fl2r](G)p.m(U)p.[fl2r](G)p | CUGUGCUGUA | |
| | .m(C)p.[fl2r](U)[n001R].m(G)p.[fl2r](U)p.[fl2r](A) | ACACAAGUAG | |
| | p.[fl2r](A)p.m(C)p.[fl2r](A)p.m(C)p.[fl2r](A)[n001 | A | |
| | R].m(A)p.[fl2r](G)p.m(U)p.[fl2r](A)p.m(G)[Ssp].[fl | | |
| | 2r](A)}\|CHEM1{[nC6o]}\|CHEM2{[Bardoyl]}$CHEM1,RNA1, | | |
| | 1:R1-1:R1\|CHEM2,CHEM1,1:R1-1:R2$$$V2.0 | | |
| SSR-0106679 | RNA1{p.m(C)[Ssp].m(U)p.[fl2r](G)p.m(U)p.[fl2r](G)p | CUGUGCUGUA | |
| | .m(C)p.[fl2r](U)[n001R].m(G)p.[fl2r](U)p.[fl2r](A) | ACACAAGUAG | |
| | p.[fl2r](A)p.m(C)p.[fl2r](A)p.m(C)p.[fl2r](A)[n001 | A | |
| | R].m(A)p.[fl2r](G)p.m(U)p.[fl2r](A)p.m(G)[Ssp].[fl | | |
| | 2r](A)}\|CHEM1{[nC6o]}\|CHEM2{[anis3C5oyl]}$CHEM1,RN | | |
| | A1,1:R1-1:R1\|CHEM2,CHEM1,1:R1-1:R2$$$V2.0 | | |
| SSR-0107358 | RNA1{m(U)[Ssp].[fl2r](U)[Rsp].m(A)p.[fl2r](U)p.m(A | UUAUAGAGCA | |
| | )p.[fl2r](G)p.m(A)p.m(G)p.m(C)p.m(A)[n001S].[fl2r] | AGAACACUGU | |
| | (A)p.m(G)p.m(A)p.[fl2r](A)p.m(C)p.[fl2r](A)p.m(C)p | UUU | |
| | .m(U)p.m(G)p.m(U)p.m(U)[Ssp].m(U)[Ssp].m(U)}$$$$V2 | | |
| | .0 | | |
| SSR-0107359 | RNA1{m(U)[Ssp].[fl2r](U)[Rsp].[Rm5m](A)p.[Rm5fl2r] | UUAUAGAGCA | |
| | (U)p.m(A)p.[fl2r](G)p.m(A)p.m(G)p.m(C)p.m(A)[n001S | AGAACACUGU | |
| | ].[fl2r](A)p.m(G)p.m(A)p.[fl2r](A)p.m(C)p.[fl2r](A | UUU | |
| | )p.m(C)p.m(U)p.m(G)p.m(U)p.m(U)[Ssp].m(U)[Ssp].m(U | | |
| | )}$$$$V2.0 | | |
| SSR-0107360 | RNA1{m(U)[Ssp].[fl2r](U)[Rsp].[Sm5m](A)p.[Sm5fl2r] | UUAUAGAGCA | |
| | (U)p.m(A)p.[fl2r](G)p.m(A)p.m(G)p.m(C)p.m(A)[n001S | AGAACACUGU | |
| | ].[fl2r](A)p.m(G)p.m(A)p.[fl2r](A)p.m(C)p.[fl2r](A | UUU | |
| | )p.m(C)p.m(U)p.m(G)p.m(U)p.m(U)[Ssp].m(U)[Ssp].m(U | | |
| | )}$$$$V2.0 | | |
| SSR-0106644 | RNA1{m(U)[Ssp].[fl2r](U)[Rsp].m(A)[n001S].[Rm5fl2r | UUAUAGAGCA | |
| | ](U)p.m(A)p.[fl2r](G)p.m(A)p.m(G)p.m(C)p.m(A)[n001 | AGAACACUGU | |
| | S].[fl2r](A)p.m(G)p.m(A)p.[fl2r](A)p.m(C)p.[fl2r]( | UUU | |
| | A)p.m(C)p.m(U)p.m(G)p.m(U)p.m(U)[Ssp].m(U)[Ssp].m( | | |
| | U)}$$$$V2.0 | | |
| SSR-0106645 | RNA1{m(U)[Ssp].[fl2r](U)[Rsp].m(A)[n001S].[Sm5fl2r | UUAUAGAGCA | |
| | ](U)p.m(A)p.[fl2r](G)p.m(A)p.m(G)p.m(C)p.m(A)[n001 | AGAACACUGU | |
| | S].[fl2r](A)p.m(G)p.m(A)p.[fl2r](A)p.m(C)p.[fl2r]( | UUU | |
| | A)p.m(C)p.m(U)p.m(G)p.m(U)p.m(U)[Ssp].m(U)[Ssp].m( | | |
| | U)}$$$$V2.0 | | |
| SSR-0106646 | RNA1{m(U)[Ssp].[fl2r](U)[Rsp].[Rm5m](A)[n001S].[fl | UUAUAGAGCA | |
| | 2r](U)p.m(A)p.[fl2r](G)p.m(A)p.m(G)p.m(C)p.m(A)[n0 | AGAACACUGU | |
| | 01S].[fl2r](A)p.m(G)p.m(A)p.[fl2r](A)p.m(C)p.[fl2r | UUU | |
| | ](A)p.m(C)p.m(U)p.m(G)p.m(U)p.m(U)[Ssp].m(U)[Ssp]. | | |
| | m(U)}$$$$V2.0 | | |
| SSR-0106648 | RNA1{m(U)[Ssp].[fl2r](U)[Ssp].[Rm5m](A)[n001S].[Rm | UUAUAGAGCA | |
| | 5fl2r](U)p.m(A)p.[fl2r](G)p.m(A)p.m(G)p.m(C)p.m(A) | AGAACACUGU | |
| | [n001S].[fl2r](A)p.m(G)p.m(A)p.[fl2r](A)p.m(C)p.[f | UUU | |
| | l2r](A)p.m(C)p.m(U)p.m(G)p.m(U)p.m(U)[Ssp].m(U)[Ss | | |
| | p].m(U)}$$$$V2.0 | | |
| SSR-0106649 | RNA1{m(U)[Ssp].[fl2r](U)[Ssp].[Sm5m](A)[n001S].[Sm | UUAUAGAGCA | |
| | 5fl2r](U)p.m(A)p.[fl2r](G)p.m(A)p.m(G)p.m(C)p.m(A) | AGAACACUGU | |
| | [n001S].[fl2r](A)p.m(G)p.m(A)p.[fl2r](A)p.m(C)p.[f | UUU | |
| | l2r](A)p.m(C)p.m(U)p.m(G)p.m(U)p.m(U)[Ssp].m(U)[Ss | | |
| | p].m(U)}$$$$V2.0 | | |
| SSR-0106650 | RNA1{m(U)[Ssp].[fl2r](U)[Ssp].m(A)[n001S].[Sgna](U | UUAUAGAGCA | |
| | )p.m(A)p.[fl2r](G)p.m(A)p.m(G)p.m(C)p.m(A)[n001S]. | AGAACACUGU | |
| | [fl2r](A)p.m(G)p.m(A)p.[fl2r](A)p.m(C)p.[fl2r](A)p | UUU | |
| | .m(C)p.m(U)p.m(G)p.m(U)p.m(U)[Ssp].m(U)[Ssp].m(U)} | | |
| | $$$$V2.0 | | |
| SSR-0107361 | RNA1{m(U)[Ssp].[fl2r](U)[Rsp].m(A)[n001S].[fl2r](U | UUAUAGAGCA | |
| | )p.m(A)p.[fl2r](G)p.m(A)p.[Sgna](G)p.m(C)p.m(A)[n0 | AGAACACUGU | |
| | 01S].[fl2r](A)p.m(G)p.m(A)p.[fl2r](A)p.m(C)p.[fl2r | UUU | |
| | ](A)p.m(C)p.m(U)p.m(G)p.m(U)p.m(U)[Ssp].m(U)[Ssp]. | | |
| | m(U)}$$$$V2.0 | | |
| SSR-0107362 | RNA1{m(U)[Ssp].[fl2r](U)[Rsp].m(A)[n001S].[fl2r](U | UUAUAGAGCA | |
| | )p.m(A)p.[fl2r](G)p.[Sgna](A)p.m(G)p.m(C)p.m(A)[n0 | AGAACACUGU | |
| | 01S].[fl2r](A)p.m(G)p.m(A)p.[fl2r](A)p.m(C)p.[fl2r | UUU | |
| | ](A)p.m(C)p.m(U)p.m(G)p.m(U)p.m(U)[Ssp].m(U)[Ssp]. | | |
| | m(U)}$$$$V2.0 | | |
| SSR-0107363 | RNA1{m(U)[Ssp].[fl2r](U)[Rsp].m(A)[n001S].[fl2r](U | UUAUAGAGCA | |
| | )p.m(A)p.[Sgna](G)p.m(A)p.m(G)p.m(C)p.m(A)[n001S]. | AGAACACUGU | |
| | [fl2r](A)p.m(G)p.m(A)p.[fl2r](A)p.m(C)p.[fl2r](A)p | UUU | |
| | .m(C)p.m(U)p.m(G)p.m(U)p.m(U)[Ssp].m(U)[Ssp].m(U)} | | |
| | $$$$V2.0 | | |
| SSR-0107364 | RNA1{m(U)[Ssp].[fl2r](U)[Rsp].m(A)[n001S].[fl2r](U | UUAUAGAGCA | |
| | )p.[Sgna](A)p.[fl2r](G)p.m(A)p.m(G)p.m(C)p.m(A)[n0 | AGAACACUGU | |
| | 01S].[fl2r](A)p.m(G)p.m(A)p.[fl2r](A)p.m(C)p.[fl2r | UUU | |
| | ](A)p.m(C)p.m(U)p.m(G)p.m(U)p.m(U)[Ssp].m(U)[Ssp]. | | |
| | m(U)}$$$$V2.0 | | |
| SSR-0107416 | RNA1{m(U)[Ssp].[fl2r](U)[Rsp].m(A)[n001S].[Sgna](T | UUATAGAGCA | |
| | )p.m(A)p.[fl2r](G)p.m(A)p.m(G)p.m(C)p.m(A)[n001S]. | AGAACACUGU | |
| | [fl2r](A)p.m(G)p.m(A)p.[fl2r](A)p.m(C)p.[fl2r](A)p | UUU | |
| | .m(C)p.m(U)p.m(G)p.m(U)p.m(U)[Ssp].m(U)[Ssp].m(U)} | | |
| | $$$$V2.0 | | |
| SSR-0107366 | RNA1{m(U)[Ssp].[fl2r](U)[Rsp].[Sgna](A)[n001S].[fl | UUAUAGAGCA | |
| | 2r](U)p.m(A)p.[fl2r](G)p.m(A)p.m(G)p.m(C)p.m(A)[n0 | AGAACACUGU | |
| | 01S].[fl2r](A)p.m(G)p.m(A)p.[fl2r](A)p.m(C)p.[fl2r | UUU | |
| | ](A)p.m(C)p.m(U)p.m(G)p.m(U)p.m(U)[Ssp].m(U)[Ssp]. | | |
| | m(U)}$$$$V2.0 | | |
| SSR-0107417 | RNA1{m(U)[Ssp].[Sgna](T)[Rsp].m(A)[n001S].[fl2r](U | UTAUAGAGCA | |
| | )p.m(A)p.[fl2r](G)p.m(A)p.m(G)p.m(C)p.m(A)[n001S]. | AGAACACUGU | |
| | [fl2r](A)p.m(G)p.m(A)p.[fl2r](A)p.m(C)p.[fl2r](A)p | UUU | |
| | .m(C)p.m(U)p.m(G)p.m(U)p.m(U)[Ssp].m(U)[Ssp].m(U)} | | |
| | $$$$V2.0 | | |
| SSR-0107418 | RNA1{[Sgna](T)[Ssp].[fl2r](U)[Rsp].m(A)[n001S].[fl | TUAUAGAGCA | |
| | 2r](U)p.m(A)p.[fl2r](G)p.m(A)p.m(G)p.m(C)p.m(A)[n0 | AGAACACUGU | |
| | 01S].[fl2r](A)p.m(G)p.m(A)p.[fl2r](A)p.m(C)p.[fl2r | UUU | |
| | ](A)p.m(C)p.m(U)p.m(G)p.m(U)p.m(U)[Ssp].m(U)[Ssp]. | | |
| | m(U)}$$$$V2.0 | | |
| SSR-0107369 | RNA1{m(U)[Ssp].[fl2r](U)[Rsp].m(A)[n001S].[fl2r](U | UUAUAGAGCA | |
| | )p.m(A)p.[fl2r](G)p.m(A)p.d(G)p.m(C)p.m(A)[n001S]. | AGAACACUGU | |
| | [fl2r](A)p.m(G)p.m(A)p.[fl2r](A)p.m(C)p.[fl2r](A)p | UUU | |
| | .m(C)p.m(U)p.m(G)p.m(U)p.m(U)[Ssp].m(U)[Ssp].m(U)} | | |
| | $$$$V2.0 | | |
| SSR-0107370 | RNA1{m(U)[Ssp].[fl2r](U)[Rsp].m(A)[n001S].[fl2r](U | UUAUAGAGCA | |
| | )p.m(A)p.[fl2r](G)p.d(A)p.m(G)p.m(C)p.m(A)[n001S]. | AGAACACUGU | |
| | [fl2r](A)p.m(G)p.m(A)p.[fl2r](A)p.m(C)p.[fl2r](A)p | UUU | |
| | .m(C)p.m(U)p.m(G)p.m(U)p.m(U)[Ssp].m(U)[Ssp].m(U)} | | |
| | $$$$V2.0 | | |
| SSR-0107371 | RNA1{m(U)[Ssp].[fl2r](U)[Rsp].m(A)[n001S].[fl2r](U | UUAUAGAGCA | |
| | )p.m(A)p.d(G)p.m(A)p.m(G)p.m(C)p.m(A)[n001S].[fl2r | AGAACACUGU | |
| | ](A)p.m(G)p.m(A)p.[fl2r](A)p.m(C)p.[fl2r](A)p.m(C) | UUU | |
| | p.m(U)p.m(G)p.m(U)p.m(U)[Ssp].m(U)[Ssp].m(U)}$$$$V | | |
| | 2.0 | | |
| SSR-0107372 | RNA1{m(U)[Ssp].[fl2r](U)[Rsp].m(A)[n001S].[fl2r](U | UUAUAGAGCA | |
| | )p.d(A)p.[fl2r](G)p.m(A)p.m(G)p.m(C)p.m(A)[n001S]. | AGAACACUGU | |
| | [fl2r](A)p.m(G)p.m(A)p.[fl2r](A)p.m(C)p.[fl2r](A)p | UUU | |
| | .m(C)p.m(U)p.m(G)p.m(U)p.m(U)[Ssp].m(U)[Ssp].m(U)} | | |
| | $$$$V2.0 | | |
| SSR-0107373 | RNA1{m(U)[Ssp].[fl2r](U)[Rsp].m(A)[n001S].d(T)p.m( | UUATAGAGCA | |
| | A)p.[fl2r](G)p.m(A)p.m(G)p.m(C)p.m(A)[n001S].[fl2r | AGAACACUGU | |
| | ](A)p.m(G)p.m(A)p.[fl2r](A)p.m(C)p.[fl2r](A)p.m(C) | UUU | |
| | p.m(U)p.m(G)p.m(U)p.m(U)[Ssp].m(U)[Ssp].m(U)}$$$$V | | |
| | 2.0 | | |
| SSR-0107374 | RNA1{m(U)[Ssp].[fl2r](U)[Rsp].d(A)[n001S].[fl2r](U | UUAUAGAGCA | |
| | )p.m(A)p.[fl2r](G)p.m(A)p.m(G)p.m(C)p.m(A)[n001S]. | AGAACACUGU | |
| | [fl2r](A)p.m(G)p.m(A)p.[fl2r](A)p.m(C)p.[fl2r](A)p | UUU | |
| | .m(C)p.m(U)p.m(G)p.m(U)p.m(U)[Ssp].m(U)[Ssp].m(U)} | | |
| | $$$$V2.0 | | |
| SSR-0107375 | RNA1{m(U)[Ssp].d(T)[Rsp].m(A)[n001S].[fl2r](U)p.m( | UTAUAGAGCA | |
| | A)p.[fl2r](G)p.m(A)p.m(G)p.m(C)p.m(A)[n001S].[fl2r | AGAACACUGU | |
| | ](A)p.m(G)p.m(A)p.[fl2r](A)p.m(C)p.[fl2r](A)p.m(C) | UUU | |
| | p.m(U)p.m(G)p.m(U)p.m(U)[Ssp].m(U)[Ssp].m(U)}$$$$V | | |
| | 2.0 | | |
| SSR-0107376 | RNA1{d(T)[Ssp].[fl2r](U)[Rsp].m(A)[n001S].[fl2r](U | TUAUAGAGCA | |
| | )p.m(A)p.[fl2r](G)p.m(A)p.m(G)p.m(C)p.m(A)[n001S]. | AGAACACUGU | |
| | [fl2r](A)p.m(G)p.m(A)p.[fl2r](A)p.m(C)p.[fl2r](A)p | UUU | |
| | .m(C)p.m(U)p.m(G)p.m(U)p.m(U)[Ssp].m(U)[Ssp].m(U)} | | |
| | $$$$V2.0 | | |
| SSR-0107377 | RNA1{m(U)[Ssp].[fl2r](U)[Rsp].m(A)[n001S].[fl2r](U | UUAUAGAGCA | |
| | )p.m(A)p.[fl2r](G)p.m(A)p.[moe](G)p.m(C)p.m(A)[n00 | AGAACACUGU | |
| | 1S].[fl2r](A)p.m(G)p.m(A)p.[fl2r](A)p.m(C)p.[fl2r] | UUU | |
| | (A)p.m(C)p.m(U)p.m(G)p.m(U)p.m(U)[Ssp].m(U)[Ssp].m | | |
| | (U)}$$$$V2.0 | | |
| SSR-0107378 | RNA1{m(U)[Ssp].[fl2r](U)[Rsp].m(A)[n001S].[fl2r](U | UUAUAGAGCA | |
| | )p.m(A)p.[fl2r](G)p.[moe](A)p.m(G)p.m(C)p.m(A)[n00 | AGAACACUGU | |
| | 1S].[fl2r](A)p.m(G)p.m(A)p.[fl2r](A)p.m(C)p.[fl2r] | UUU | |
| | (A)p.m(C)p.m(U)p.m(G)p.m(U)p.m(U)[Ssp].m(U)[Ssp].m | | |
| | (U)}$$$$V2.0 | | |
| SSR-0107379 | RNA1{m(U)[Ssp].[fl2r](U)[Rsp].m(A)[n001S].[fl2r](U | UUAUAGAGCA | |
| | )p.m(A)p.[moe](G)p.m(A)p.m(G)p.m(C)p.m(A)[n001S].[ | AGAACACUGU | |
| | fl2r](A)p.m(G)p.m(A)p.[fl2r](A)p.m(C)p.[fl2r](A)p. | UUU | |
| | m(C)p.m(U)p.m(G)p.m(U)p.m(U)[Ssp].m(U)[Ssp].m(U)}$ | | |
| | $$$V2.0 | | |
| SSR-0107380 | RNA1{m(U)[Ssp].[fl2r](U)[Rsp].m(A)[n001S].[fl2r](U | UUAUAGAGCA | |
| | )p.[moe](A)p.[fl2r](G)p.m(A)p.m(G)p.m(C)p.m(A)[n00 | AGAACACUGU | |
| | 1S].[fl2r](A)p.m(G)p.m(A)p.[fl2r](A)p.m(C)p.[fl2r] | UUU | |
| | (A)p.m(C)p.m(U)p.m(G)p.m(U)p.m(U)[Ssp].m(U)[Ssp].m | | |
| | (U)}$$$$V2.0 | | |
| SSR-0107381 | RNA1{m(U)[Ssp].[fl2r](U)[Rsp].m(A)[n001S].[moe](T) | UUATAGAGCA | |
| | p.m(A)p.[fl2r](G)p.m(A)p.m(G)p.m(C)p.m(A)[n001S].[ | AGAACACUGU | |
| | fl2r](A)p.m(G)p.m(A)p.[fl2r](A)p.m(C)p.[fl2r](A)p. | UUU | |
| | m(C)p.m(U)p.m(G)p.m(U)p.m(U)[Ssp].m(U)[Ssp].m(U)}$ | | |
| | $$$V2.0 | | |
| SSR-0107382 | RNA1{m(U)[Ssp].[fl2r](U)[Rsp].[moe](A)[n001S].[fl2 | UUAUAGAGCA | |
| | r](U)p.m(A)p.[fl2r](G)p.m(A)p.m(G)p.m(C)p.m(A)[n00 | AGAACACUGU | |
| | 1S].[fl2r](A)p.m(G)p.m(A)p.[fl2r](A)p.m(C)p.[fl2r] | UUU | |
| | (A)p.m(C)p.m(U)p.m(G)p.m(U)p.m(U)[Ssp].m(U)[Ssp].m | | |
| | (U)}$$$$V2.0 | | |
| SSR-0107383 | RNA1{m(U)[Ssp].[moe](T)[Rsp].m(A)[n001S].[fl2r](U) | UTAUAGAGCA | |
| | p.m(A)p.[fl2r](G)p.m(A)p.m(G)p.m(C)p.m(A)[n001S].[ | AGAACACUGU | |
| | fl2r](A)p.m(G)p.m(A)p.[fl2r](A)p.m(C)p.[fl2r](A)p. | UUU | |
| | m(C)p.m(U)p.m(G)p.m(U)p.m(U)[Ssp].m(U)[Ssp].m(U)}$ | | |
| | $$$V2.0 | | |
| SSR-0107384 | RNA1{[moe](T)[Ssp].[fl2r](U)[Rsp].m(A)[n001S].[fl2 | TUAUAGAGCA | |
| | r](U)p.m(A)p.[fl2r](G)p.m(A)p.m(G)p.m(C)p.m(A)[n00 | AGAACACUGU | |
| | 1S].[fl2r](A)p.m(G)p.m(A)p.[fl2r](A)p.m(C)p.[fl2r] | UUU | |
| | (A)p.m(C)p.m(U)p.m(G)p.m(U)p.m(U)[Ssp].m(U)[Ssp].m | | |
| | (U)}$$$$V2.0 | | |
| SSR-0107385 | RNA1{m(U)[Ssp].[fl2r](U)[Rsp].m(A)[n001S].[fl2r](U | UUAUAGAGCA | |
| | )p.m(A)p.[fl2r](G)p.m(A)p.[Ir](G)p.m(C)p.m(A)[n001 | AGAACACUGU | |
| | S].[fl2r](A)p.m(G)p.m(A)p.[fl2r](A)p.m(C)p.[fl2r]( | UUU | |
| | A)p.m(C)p.m(U)p.m(G)p.m(U)p.m(U)[Ssp].m(U)[Ssp].m( | | |
| | U)}$$$$V2.0 | | |
| SSR-0107386 | RNA1{m(U)[Ssp].[fl2r](U)[Rsp].m(A)[n001S].[fl2r](U | UUAUAGAGCA | |
| | )p.m(A)p.[fl2r](G)p.[Ir](A)p.m(G)p.m(C)p.m(A)[n001 | AGAACACUGU | |
| | S].[fl2r](A)p.m(G)p.m(A)p.[fl2r](A)p.m(C)p.[fl2r]( | UUU | |
| | A)p.m(C)p.m(U)p.m(G)p.m(U)p.m(U)[Ssp].m(U)[Ssp].m( | | |
| | U)}$$$$V2.0 | | |
| SSR-0107387 | RNA1{m(U)[Ssp].[fl2r](U)[Rsp].m(A)[n001S].[fl2r](U | UUAUAGAGCA | |
| | )p.m(A)p.[Ir](G)p.m(A)p.m(G)p.m(C)p.m(A)[n001S].[f | AGAACACUGU | |
| | l2r](A)p.m(G)p.m(A)p.[fl2r](A)p.m(C)p.[fl2r](A)p.m | UUU | |
| | (C)p.m(U)p.m(G)p.m(U)p.m(U)[Ssp].m(U)[Ssp].m(U)}$$ | | |
| | $$V2.0 | | |
| SSR-0107388 | RNA1{m(U)[Ssp].[fl2r](U)[Rsp].m(A)[n001S].[fl2r](U | UUAUAGAGCA | |
| | )p.[Ir](A)p.[fl2r](G)p.m(A)p.m(G)p.m(C)p.m(A)[n001 | AGAACACUGU | |
| | S].[fl2r](A)p.m(G)p.m(A)p.[fl2r](A)p.m(C)p.[fl2r]( | UUU | |
| | A)p.m(C)p.m(U)p.m(G)p.m(U)p.m(U)[Ssp].m(U)[Ssp].m( | | |
| | U)}$$$$V2.0 | | |
| SSR-0107389 | RNA1{m(U)[Ssp].[fl2r](U)[Rsp].m(A)[n001S].[Ir](T)p | UUATAGAGCA | |
| | .m(A)p.[fl2r](G)p.m(A)p.m(G)p.m(C)p.m(A)[n001S].[f | AGAACACUGU | |
| | l2r](A)p.m(G)p.m(A)p.[fl2r](A)p.m(C)p.[fl2r](A)p.m | UUU | |
| | (C)p.m(U)p.m(G)p.m(U)p.m(U)[Ssp].m(U)[Ssp].m(U)}$$ | | |
| | $$V2.0 | | |
| SSR-0107390 | RNA1{m(U)[Ssp].[fl2r](U)[Rsp].[Ir](A)[n001S].[fl2r | UUAUAGAGCA | |
| | ](U)p.m(A)p.[fl2r](G)p.m(A)p.m(G)p.m(C)p.m(A)[n001 | AGAACACUGU | |
| | S].[fl2r](A)p.m(G)p.m(A)p.[fl2r](A)p.m(C)p.[fl2r]( | UUU | |
| | A)p.m(C)p.m(U)p.m(G)p.m(U)p.m(U)[Ssp].m(U)[Ssp].m( | | |
| | U)}$$$$V2.0 | | |
| SSR-0107391 | RNA1{m(U)[Ssp].[Ir](T)[Rsp].m(A)[n001S].[fl2r](U)p | UTAUAGAGCA | |
| | .m(A)p.[fl2r](G)p.m(A)p.m(G)p.m(C)p.m(A)[n001S].[f | AGAACACUGU | |
| | l2r](A)p.m(G)p.m(A)p.[fl2r](A)p.m(C)p.[fl2r](A)p.m | UUU | |
| | (C)p.m(U)p.m(G)p.m(U)p.m(U)[Ssp].m(U)[Ssp].m(U)}$$ | | |
| | $$V2.0 | | |
| SSR-0107392 | RNA1{[Ir](T)[Ssp].[fl2r](U)[Rsp].m(A)[n001S].[fl2r | TUAUAGAGCA | |
| | ](U)p.m(A)p.[fl2r](G)p.m(A)p.m(G)p.m(C)p.m(A)[n001 | AGAACACUGU | |
| | S].[fl2r](A)p.m(G)p.m(A)p.[fl2r](A)p.m(C)p.[fl2r]( | UUU | |
| | A)p.m(C)p.m(U)p.m(G)p.m(U)p.m(U)[Ssp].m(U)[Ssp].m( | | |
| | U)}$$$$V2.0 | | |
| SSR-0106661 | RNA1{[oC60][n009].m(C)[Ssp].m(U)p.[fl2r](G)p.m(U)p | CUGUGCUGUA | |
| | .[fl2r](G)p.m(C)p.[fl2r](U)[n001R].m(G)p.[fl2r](U) | ACACAAGUAG | |
| | p.[fl2r](A)p.[fl2r](A)p.m(C)p.[fl2r](A)p.m(C)p.[fl | A | |
| | 2r](A)[n001R].m(A)p.[fl2r](G)p.m(U)p.[fl2r](A)p.m( | | |
| | G)[Ssp].[fl2r](A)}$$$$V2.0 | | |
| SSR-0106662 | RNA1{[oC60][n033].m(C)[Ssp].m(U)p.[fl2r](G)p.m(U)p | CUGUGCUGUA | |
| | .[fl2r](G)p.m(C)p.[fl2r](U)[n001R].m(G)p.[fl2r](U) | ACACAAGUAG | |
| | p.[fl2r](A)p.[fl2r](A)p.m(C)p.[fl2r](A)p.m(C)p.[fl | A | |
| | 2r](A)[n001R].m(A)p.[fl2r](G)p.m(U)p.[fl2r](A)p.m( | | |
| | G)[Ssp].[fl2r](A)}$$$$V2.0 | | |
| SSR-0106663 | RNA1{[oC60][n039].m(C)[Ssp].m(U)p.[fl2r](G)p.m(U)p | CUGUGCUGUA | |
| | .[fl2r](G)p.m(C)p.[fl2r](U)[n001R].m(G)p.[fl2r](U) | ACACAAGUAG | |
| | p.[fl2r](A)p.[fl2r](A)p.m(C)p.[fl2r](A)p.m(C)p.[fl | A | |
| | 2r](A)[n001R].m(A)p.[fl2r](G)p.m(U)p.[fl2r](A)p.m( | | |
| | G)[Ssp].[fl2r](A)}$$$$V2.0 | | |
| SSR-0106664 | RNA1{[oC60][n040].m(C)[Ssp].m(U)p.[fl2r](G)p.m(U)p | CUGUGCUGUA | |
| | .[fl2r](G)p.m(C)p.[fl2r](U)[n001R].m(G)p.[fl2r](U) | ACACAAGUAG | |
| | p.[fl2r](A)p.[fl2r](A)p.m(C)p.[fl2r](A)p.m(C)p.[fl | A | |
| | 2r](A)[n001R].m(A)p.[fl2r](G)p.m(U)p.[fl2r](A)p.m( | | |
| | G)[Ssp].[fl2r](A)}$$$$V2.0 | | |
| SSR-0106665 | RNA1{m(C)[Ssp].m(U)p.[fl2r](G)p.m(U)p.[fl2r](G)p.m | CUGUGCUGUA | |
| | (C)p.[fl2r](U)[n039R].m(G)p.[fl2r](U)p.[fl2r](A)p. | ACACAAGUAG | |
| | [fl2r](A)p.m(C)p.[fl2r](A)p.m(C)p.[fl2r](A)p.m(A)p | A | |
| | .[fl2r](G)p.m(U)p.[fl2r](A)p.m(G)[Ssp].[fl2r](A)}$ | | |
| | $$$V2.0 | | |
| SSR-0106666 | RNA1{m(C)[Ssp].m(U)p.[fl2r](G)p.m(U)p.[fl2r](G)p.m | CUGUGCUGUA | |
| | (C)p.[fl2r](U)[n040R].m(G)p.[fl2r](U)p.[fl2r](A)p. | ACACAAGUAG | |
| | [fl2r](A)p.m(C)p.[fl2r](A)p.m(C)p.[fl2r](A)p.m(A)p | A | |
| | .[fl2r](G)p.m(U)p.[fl2r](A)p.m(G)[Ssp].[fl2r](A)}$ | | |
| | $$$V2.0 | | |
| SSR-0106601 | RNA1{m(C)[Ssp].m(U)p.[fl2r](G)p.m(U)p.[fl2r](G)p.m | CUGUGCUGUA | |
| | (C)p.[hexdec2r](U)[n009R].m(G)p.[fl2r](U)p.[fl2r]( | ACACAAGUAG | |
| | A)p.[fl2r](A)p.m(C)p.[fl2r](A)p.m(C)p.[fl2r](A)p.m | A | |
| | (A)p.[fl2r](G)p.m(U)p.[fl2r](A)p.m(G)[Ssp].[fl2r]( | | |
| | A)}$$$$V2.0 | | |
| SSR-0106602 | RNA1{m(C)[Ssp].m(U)p.[fl2r](G)p.m(U)p.[fl2r](G)p.m | CUGUGCUGUA | |
| | (C)p.[hexdec2r](U)[n033R].m(G)p.[fl2r](U)p.[fl2r]( | ACACAAGUAG | |
| | A)p.[fl2r](A)p.m(C)p.[fl2r](A)p.m(C)p.[fl2r](A)p.m | A | |
| | (A)p.[fl2r](G)p.m(U)p.[fl2r](A)p.m(G)[Ssp].[fl2r]( | | |
| | A)}$$$$V2.0 | | |

TABLE 1d
Example Guide Strand Compositions.

| ID | HELM | BASE SEQUENCE | NAME |
|---|---|---|---|
| SSR-0104100 | RNA1{m(U)[sp].[fl2r](C)[sp].m(U)p.[fl2r](A)p.m(C)p.[fl2r](U)p.m(U)p.[fl2r](G)p.m(U)p.[fl2r](G)p.m(U)p. | UCUACUUGUGUUA | WV-50670 |
| | m(U)p.m(A)p.[fl2r](C)p.m(A)p.[fl2r](G)p.m(C)p.[fl2r](A)p.m(C)p.[fl2r](A)p.m(G)[sp].m(U)[sp].m(U)}$$$$ | CAGCACAGUU | |
| | V2.0 | | |
| SSR-0104186 | RNA1{p.[Rm5d5m](U)[Rsp].[fl2r](U)[Ssp].m(A)[n001S].[fl2r](U)p.m(A)p.[fl2r](G)p.m(A)p.m(G)p.m(C)p. | UUAUAGAGCAAGA | WV-50105 |
| | m(A)[n001R].[fl2r](A)p.m(G)p.m(A)p.[fl2r](A)p.m(C)p.[fl2r](A)p.m(C)p.m(U)p.m(G)p.m(U)p.m(U)[Ssp].m | ACACUGUUUU | |
| | (U)[Ssp].m(U)}$$$$V2.0 | | |
| SSR-0104189 | RNA1{p.[Rm5d5m](U)[Ssp].[fl2r](U)[Rsp].m(A)[n001S].[fl2r](U)p.m(A)p.[fl2r](G)p.m(A)p.m(G)p.m(C)p. | UUAUAGAGCAAGA | WV-50106 |
| | m(A)[n001R].[fl2r](A)p.m(G)p.m(A)p.[fl2r](A)p.m(C)p.[fl2r](A)p.m(C)p.m(U)p.m(G)p.m(U)p.m(U)[Ssp].m | ACACUGUUUU | |
| | (U)[Ssp].m(U)}$$$$V2.0 | | |
| SSR-0104473 | RNA1{m(U)[Ssp].[fl2r](U)[Rsp].m(A)[n001S].[fl2r](U)p.m(A)p.[fl2r](G)p.m(A)p.m(G)p.m(C)p.m(A)[n001R | UUAUAGAGCAAGA | WV-48528 |
| | ].[fl2r](A)p.m(G)p.m(A)p.[fl2r](A)p.m(C)p.[fl2r](A)p.m(C)p.m(U)p.m(G)p.m(U)p.m(U)[Ssp].m(U)[Ssp].m( | ACACUGUUUU | |
| | U)}$$$$V2.0 | | |
| SSR-0104475 | RNA1{m(U)[Ssp].[fl2r](U)[Rsp].m(A)[n001S].[fl2r](U)p.m(A)p.[fl2r](G)p.m(A)p.m(G)p.m(C)p.m(A)[n001S | UUAUAGAGCAAGA | WV-47145 |
| | ].[fl2r](A)p.m(G)p.m(A)p.[fl2r](A)p.m(C)p.[fl2r](A)p.m(C)p.m(U)p.m(G)p.m(U)p.m(U)[Ssp].m(U)[Ssp].m( | ACACUGUUUU | |
| | U)}$$$$V2.0 | | |
| SSR-0104717 | RNA1{m(U)[sp].[fl2r](U)[sp].m(A)p.[fl2r](U)p.m(A)p.[fl2r](G)p.m(A)p.m(G)p.m(C)p.m(A)p.[fl2r](A)p.m(G | UUAUAGAGCAAGA | WV-49611 |
| | )p.m(A)p.[fl2r](A)p.m(C)p.[fl2r](A)p.m(C)p.m(U)p.m(G)p.m(U)p.m(U)[sp].m(U)[sp].m(U)}$$$$V2.0 | ACACUGUUUU | |
| SSR-0105217 | RNA1{m(U)[Ssp].[fl2r](C)[Rsp].m(C)[n001S].[fl2r](U)p.m(U)p.[fl2r](C)p.m(C)p.m(C)p.m(U)p.m(G)[n001R] | UCCUUCCCUGAAG | WV-49614 |
| | .[fl2r](A)p.m(A)p.m(G)p.[fl2r](G)p.m(U)p.[fl2r](U)p.m(C)p.m(C)p.m(U)p.m(C)p.m(C)[Ssp].m(U)[Ssp].m(U | GUUCCUCCUU | |
| | )}$$$$V2.0 | | |
| SSR-0105245 | RNA1{m(U)[Ssp].[fl2r](U)[Rsp].m(A)p.[fl2r](U)p.m(A)p.[fl2r](G)p.m(A)p.m(G)p.m(C)p.m(A)p.[fl2r](A)p.m | UUAUAGAGCAAGA | WV-51122 |
| | (G)p.m(A)p.[fl2r](A)p.m(C)p.[fl2r](A)p.m(C)p.m(U)p.m(G)p.m(U)p.m(U)[Ssp].m(U)[Ssp].m(U)}$$$$V2.0 | ACACUGUUUU | |
| SSR-0106183 | RNA1{p.[Rm5d5m](U)[Rsp].[fl2r](C)[Ssp].m(U)[n001S].[fl2r](A)p.m(C)p.[fl2r](U)p.m(U)[n001S].[fl2r](G)p | UCUACUUGUGUUA | |
| | .m(U)p.[fl2r](G)p.m(U)p.m(U)p.m(A)[Ssp].[fl2r](C)[Ssp].m(A)[Ssp].[fl2r](G)[Ssp].m(C)[Ssp].[fl2r](A) | CAGCACAGUU | |
| | [n001S].m(C)[Ssp].[fl2r](A)[Ssp].m(G)[Ssp].m(U)[Ssp].m(U)}$$$$V2.0 | | |
| SSR-0106224 | RNA1{p.[Rm5d5m](U)[Ssp].[fl2r](C)[Ssp].m(U)[n001S].[fl2r](A)p.m(C)p.[fl2r](U)p.m(U)[n001S].[fl2r](G)p | UCUACUUGUGUUA | |
| | .m(U)p.[fl2r](G)p.m(U)p.m(U)p.m(A)[Ssp].[fl2r](C)[Ssp].m(A)[Ssp].[fl2r](G)[Ssp].m(C)[Ssp].[fl2r](A) | CAGCACAGUU | |
| | [n001S].m(C)[Ssp].[fl2r](A)[Ssp].m(G)[Ssp].m(U)[Ssp].m(U)}$$$$V2.0 | | |
| SSR-0106453 | RNA1{m(U)[Ssp].[fl2r](U)[Rsp].[thpyr](A)[n001S].[fl2r](U)p.m(A)p.[fl2r](G)p.m(A)p.m(G)p.m(C)p.m(A)[n | UUAUAGAGCAAGA | |
| | 001S].[fl2r](A)p.m(G)p.m(A)p.[fl2r](A)p.m(C)p.[fl2r](A)p.m(C)p.m(U)p.m(G)p.m(U)p.m(U)[Ssp].m(U)[Ssp | ACACUGUUUU | |
| | ].m(U)}$$$$V2.0 | | |
| SSR-0106454 | RNA1{m(U)[Ssp].[fl2r](U)[Rsp].m(A)[n001S].[fl2r](U)p.[thpyr](A)p.[fl2r](G)p.m(A)p.m(G)p.m(C)p.m(A)[n | UUAUAGAGCAAGA | |
| | 001S].[fl2r](A)p.m(G)p.m(A)p.[fl2r](A)p.m(C)p.[fl2r](A)p.m(C)p.m(U)p.m(G)p.m(U)p.m(U)[Ssp].m(U)[Ssp | ACACUGUUUU | |
| | ].m(U)}$$$$V2.0 | | |
| SSR-0106455 | RNA1{m(U)[Ssp].[fl2r](U)[Rsp].m(A)[n001S].[fl2r](U)p.m(A)p.[fl2r](G)p.[thpyr](A)p.m(G)p.m(C)p.m(A)[n | UUAUAGAGCAAGA | |
| | 001S].[fl2r](A)p.m(G)p.m(A)p.[fl2r](A)p.m(C)p.[fl2r](A)p.m(C)p.m(U)p.m(G)p.m(U)p.m(U)[Ssp].m(U)[Ssp | ACACUGUUUU | |
| | ].m(U)}$$$$V2.0 | | |
| SSR-0106456 | RNA1{m(U)[Ssp].[fl2r](U)[Rsp].m(A)[n001S].[fl2r](U)p.m(A)p.[fl2r](G)p.m(A)p.m(G)p.m(C)p.[thpyr](A)[n | UUAUAGAGCAAGA | |
| | 001S].[fl2r](A)p.m(G)p.m(A)p.[fl2r](A)p.m(C)p.[fl2r](A)p.m(C)p.m(U)p.m(G)p.m(U)p.m(U)[Ssp].m(U)[Ssp | ACACUGUUUU | |
| | ].m(U)}$$$$V2.0 | | |
| SSR-0106457 | RNA1{d([3nU])[Ssp].[fl2r](U)[Rsp].m(A)[n001S].[fl2r](U)p.m(A)p.[fl2r](G)p.m(A)p.m(G)p.m(C)p.m(A)[n0 | UUAUAGAGCAAGA | |
| | 01S].[fl2r](A)p.m(G)p.m(A)p.[fl2r](A)p.m(C)p.[fl2r](A)p.m(C)p.m(U)p.m(G)p.m(U)p.m(U)[Ssp].m(U)[Ssp] | ACACUGUUUU | |
| | .m(U)}$$$$V2.0 | | |
| SSR-0106512 | RNA1{m(U)[Rsp].[fl2r](C)[Ssp].m(U)[n029S].[fl2r](A)p.m(C)p.[fl2r](U)p.m(U)[n029S].[fl2r](G)p.m(U)p. | UCUACUUGUGUUA | |
| | [fl2r](G)p.m(U)p.m(U)p.m(A)[Ssp].[fl2r](C)[Ssp].m(A)[Ssp].[fl2r](G)[Ssp].m(C)[Ssp].[fl2r](A)[n029S]. | CAGCACAGUU | |
| | m(C)[Ssp].[fl2r](A)[Ssp].m(G)[Ssp].m(U)[Ssp].m(U)}$$$$V2.0 | | |
| SSR-0106514 | RNA1{[d5m](U)[Rsp].[fl2r](C)[Ssp].m(U)[n001S].[fl2r](A)p.m(C)p.[fl2r](U)p.m(U)[n001S].[fl2r](G)p.m(U) | UCUACUUGUGUUA | |
| | p.[fl2r](G)p.m(U)p.m(U)p.m(A)[Ssp].[fl2r](C)[Ssp].m(A)[Ssp].[fl2r](G)[Ssp].m(C)[Ssp].[fl2r](A)[n001S]. | CAGCACAGUU | |
| | m(C)[Ssp].[fl2r](A)[Ssp].m(G)[Ssp].m(U)[Ssp].m(U)}\|CHEM1{[ptz]}$CHEM1, RNA1,1: R1-1: R1$$$V2.0 | | |
| SSR-0106515 | RNA1{[d5m](U)[Ssp].[fl2r](C)[Ssp].m(U)[n001S].[fl2r](A)p.m(C)p.[fl2r](U)p.m(U)[n001S].[fl2r](G)p.m(U) | UCUACUUGUGUUA | |
| | p.[fl2r](G)p.m(U)p.m(U)p.m(A)[Ssp].[fl2r](C)[Ssp].m(A)[Ssp].[fl2r](G)[Ssp].m(C)[Ssp].[fl2r](A)[n001S]. | CAGCACAGUU | |
| | m(C)[Ssp].[fl2r](A)[Ssp].m(G)[Ssp].m(U)[Ssp].m(U)}\|CHEM1{[ptz]}$CHEM1,RNA1,1:R1-1: R1$$$V2.0 | | |
| SSR-0106516 | RNA1{[vped5m](U)[Rsp].[fl2r](C)[Ssp].m(U)[n001S].[fl2r](A)p.m(C)p.[fl2r](U)p.m(U)[n001S].[fl2r](G)p.m | UCUACUUGUGUUA | |
| | (U)p.[fl2r](G)p.m(U)p.m(U)p.m(A)[Ssp].[fl2r](C)[Ssp].m(A)[Ssp].[fl2r](G)[Ssp].m(C)[Ssp].[fl2r](A) | CAGCACAGUU | |
| | [n001S].m(C)[Ssp].[fl2r](A)[Ssp].m(G)[Ssp].m(U)[Ssp].m(U)}$$$$V2.0 | | |
| SSR-0106517 | RNA1{[vped5m](U)[Ssp].[fl2r](C)[Ssp].m(U)[n001S].[fl2r](A)p.m(C)p.[fl2r](U)p.m(U)[n001S].[fl2r](G)p. | UCUACUUGUGUUA | |
| | m(U)p.[fl2r](G)p.m(U)p.m(U)p.m(A)[Ssp].[fl2r](C)[Ssp].m(A)[Ssp].[fl2r](G)[Ssp].m(C)[Ssp].[fl2r](A) | CAGCACAGUU | |
| | [n001S].m(C)[Ssp].[fl2r](A)[Ssp].m(G)[Ssp].m(U)[Ssp].m(U)}$$$$V2.0 | | |
| SSR-0106518 | RNA1{[d25r](T)[Ssp].[fl2r](C)[Ssp].m(U)[n001S].[fl2r](A)p.m(C)p.[fl2r](U)p.m(U)[n001S].[fl2r](G)p. | TCUACUUGUGUUA | |
| | m(U)p.[fl2r](G)p.m(U)p.m(U)p.m(A)[Ssp].[fl2r](C)[Ssp].m(A)[Ssp].[fl2r](G)[Ssp].m(C)[Ssp].[fl2r](A) | CAGCACAGUU | |
| | [n001S].m(C)[Ssp].[fl2r](A)[Ssp].m(G)[Ssp].m(U)[Ssp].m(U)}\|CHEM1{[ptz]}$CHEM1, RNA1,1:R1-1:R1$$$V2.0 | | |
| SSR-0106519 | RNA1{p.[Rm5d5m](U)[Rsp].[fl2r](C)[Ssp].m(U)[n029S].[fl2r](A)p.m(C)p.[fl2r](U)p.m(U)[n029S].[fl2r](G)p | UCUACUUGUGUUA | |
| | .m(U)p.[fl2r](G)p.m(U)p.m(U)p.m(A)[Ssp].[fl2r](C)[Ssp].m(A)[Ssp].[fl2r](G)[Ssp].m(C)[Ssp].[fl2r](A) | CAGCACAGUU | |
| | [n029S].m(C)[Ssp].[fl2r](A)[Ssp].m(G)[Ssp].m(U)[Ssp].m(U)}$$$$V2.0 | | |
| SSR-0106520 | RNA1{m(U)[sp].[fl2r](U)[sp].m(A)p.[fl2r](U)p.m(A)p.[fl2r](G)p.[thpyr](A)p.m(G)p.m(C)p.m(A)p.[fl2r](A)p | UUAUAGAGCAAGA | |
| | .m(G)p.m(A)p.[fl2r](A)p.m(C)p.[fl2r](A)p.m(C)p.m(U)p.m(G)p.m(U)p.m(U)[sp].m(U)[sp].m(U)}$$$$V2.0 | ACACUGUUUU | |
| SSR-0106521 | RNA1{[thpyr](T)[Ssp].[fl2r](U)[Rsp].m(A)[n001S].[fl2r](U)p.m(A)p.[fl2r](G)p.m(A)p.m(G)p.m(C)p.m(A)[n | TUAUAGAGCAAGA | |
| | 001S].[fl2r](A)p.m(G)p.m(A)p.[fl2r](A)p.m(C)p.[fl2r](A)p.m(C)p.m(U)p.m(G)p.m(U)p.m(U)[Ssp].m(U)[Ssp | ACACUGUUUU | |
| | ].m(U)}$$$$V2.0 | | |
| SSR-0106522 | RNA1{m(U)[Ssp].[thpyr](T)[Rsp].m(A)[n001S].[fl2r](U)p.m(A)p.[fl2r](G)p.m(A)p.m(G)p.m(C)p.m(A)[n001 | UTAUAGAGCAAGA | |
| | S].[fl2r](A)p.m(G)p.m(A)p.[fl2r](A)p.m(C)p.[fl2r](A)p.m(C)p.m(U)p.m(G)p.m(U)p.m(U)[Ssp].m(U)[Ssp].m | ACACUGUUUU | |
| | (U)}$$$$V2.0 | | |
| SSR-0106523 | RNA1{m(U)[Ssp].[fl2r](U)[Rsp].m(A)[n001S].[thpyr](T)p.m(A)p.[fl2r](G)p.m(A)p.m(G)p.m(C)p.m(A)[n001 | UUATAGAGCAAGA | |
| | S].[fl2r](A)p.m(G)p.m(A)p.[fl2r](A)p.m(C)p.[fl2r](A)p.m(C)p.m(U)p.m(G)p.m(U)p.m(U)[Ssp].m(U)[Ssp].m | ACACUGUUUU | |
| | (U)}$$$$V2.0 | | |
| SSR-0106524 | RNA1{m(U)[Ssp].[fl2r](U)[Rsp].m(A)[n001S].[fl2r](U)p.m(A)p.[thpyr](G)p.m(A)p.m(G)p.m(C)p.m(A)[n00 | UUAUAGAGCAAGA | |
| | 1S].[fl2r](A)p.m(G)p.m(A)p.[fl2r](A)p.m(C)p.[fl2r](A)p.m(C)p.m(U)p.m(G)p.m(U)p.m(U)[Ssp].m(U)[Ssp]. | ACACUGUUUU | |
| | m(U)}$$$$V2.0 | | |
| SSR-0106525 | RNA1{m(U)[Ssp].[fl2r](U)[Rsp].m(A)[n001S].[fl2r](U)p.m(A)p.[fl2r](G)p.m(A)p.[thpyr](G)p.m(C)p.m(A)[n | UUAUAGAGCAAGA | |
| | 001S].[fl2r](A)p.m(G)p.m(A)p.[fl2r](A)p.m(C)p.[fl2r](A)p.m(C)p.m(U)p.m(G)p.m(U)p.m(U)[Ssp].m(U)[Ssp | ACACUGUUUU | |
| | ].m(U)}$$$$V2.0 | | |
| SSR-0106526 | RNA1{m(U)[Ssp].[fl2r](U)[Rsp].m(A)[n001S].[fl2r](U)p.m(A)p.[fl2r](G)p.m(A)p.m(G)p.[thpyr](C)p.m(A)[n | UUAUAGAGCAAGA | |
| | 001S].[fl2r](A)p.m(G)p.m(A)p.[fl2r](A)p.m(C)p.[fl2r](A)p.m(C)p.m(U)p.m(G)p.m(U)p.m(U)[Ssp].m(U)[Ssp | ACACUGUUUU | |
| | ].m(U)}$$$$V2.0 | | |
| SSR-0106527 | RNA1{m(U)[Ssp].[fl2r](U)[Rsp].m(A)[n001S].[fl2r](U)p.m(A)p.[fl2r](G)p.m(A)p.m(G)p.m(C)p.m(A)[n001S | UUAUAGAGCAAGA | |
| | ].[thpyr](A)p.m(G)p.m(A)p.[fl2r](A)p.m(C)p.[fl2r](A)p.m(C)p.m(U)p.m(G)p.m(U)p.m(U)[Ssp].m(U)[Ssp]. | ACACUGUUUU | |
| | m(U)}$$$$V2.0 | | |
| SSR-0106528 | RNA1{m(U)[Ssp].[fl2r](U)[Rsp].m(A)[n001S].[fl2r](U)p.m(A)p.[fl2r](G)p.m(A)p.m(G)p.m(C)p.m(A)[n001S | UUAUAGAGCAAGA | |
| | ].[fl2r](A)p.[thpyr](G)p.m(A)p.[fl2r](A)p.m(C)p.[fl2r](A)p.m(C)p.m(U)p.m(G)p.m(U)p.m(U)[Ssp].m(U) | ACACUGUUUU | |
| | [Ssp].m(U)}$$$$V2.0 | | |
| SSR-0106529 | RNA1{m(U)[Ssp].[fl2r](U)[Rsp].m(A)[n001S].[fl2r](U)p.m(A)p.[fl2r](G)p.m(A)p.m(G)p.m(C)p.m(A)[n001S | UUAUAGAGCAAGA | |
| | ].[fl2r](A)p.m(G)p.[thpyr](A)p.[fl2r](A)p.m(C)p.[fl2r](A)p.m(C)p.m(U)p.m(G)p.m(U)p.m(U)[Ssp].m(U) | ACACUGUUUU | |
| | [Ssp].m(U)}$$$$V2.0 | | |
| SSR-0106530 | RNA1{m(U)[Ssp].[fl2r](U)[Rsp].m(A)[n001S].[fl2r](U)p.m(A)p.[fl2r](G)p.m(A)p.m(G)p.m(C)p.m(A)[n001S | UUAUAGAGCAAGA | |
| | ].[fl2r](A)p.m(G)p.m(A)p.[thpyr](A)p.m(C)p.[fl2r](A)p.m(C)p.m(U)p.m(G)p.m(U)p.m(U)[Ssp].m(U)[Ssp]. | ACACUGUUUU | |
| | m(U)}$$$$V2.0 | | |
| SSR-0106531 | RNA1{m(U)[Ssp].[fl2r](U)[Rsp].m(A)[n001S].[fl2r](U)p.m(A)p.[fl2r](G)p.m(A)p.m(G)p.m(C)p.m(A)[n001S | UUAUAGAGCAAGA | |
| | ].[fl2r](A)p.m(G)p.m(A)p.[fl2r](A)p.[thpyr](C)p.[fl2r](A)p.m(C)p.m(U)p.m(G)p.m(U)p.m(U)[Ssp].m(U) | ACACUGUUUU | |
| | [Ssp].m(U)}$$$$V2.0 | | |
| SSR-0106532 | RNA1{m(U)[Ssp].[fl2r](U)[Rsp].m(A)[n001S].[fl2r](U)p.m(A)p.[fl2r](G)p.m(A)p.m(G)p.m(C)p.m(A)[n001S | UUAUAGAGCAAGA | |
| | ].[fl2r](A)p.m(G)p.m(A)p.[fl2r](A)p.m(C)p.[thpyr](A)p.m(C)p.m(U)p.m(G)p.m(U)p.m(U)[Ssp].m(U)[Ssp]. | ACACUGUUUU | |
| | m(U)}$$$$V2.0 | | |
| SSR-0106533 | RNA1{m(U)[Ssp].[fl2r](U)[Rsp].m(A)[n001S].[fl2r](U)p.m(A)p.[fl2r](G)p.m(A)p.m(G)p.m(C)p.m(A)[n001S | UUAUAGAGCAAGA | |
| | ].[fl2r](A)p.m(G)p.m(A)p.[fl2r](A)p.m(C)p.[fl2r](A)p.[thpyr](C)p.m(U)p.m(G)p.m(U)p.m(U)[Ssp].m(U) | ACACUGUUUU | |
| | [Ssp].m(U)}$$$$V2.0 | | |
| SSR-0106534 | RNA1{m(U)[Ssp].[fl2r](U)[Rsp].m(A)[n001S].[fl2r](U)p.m(A)p.[fl2r](G)p.m(A)p.m(G)p.m(C)p.m(A)[n001S | UUAUAGAGCAAGA | |
| | ].[fl2r](A)p.m(G)p.m(A)p.[fl2r](A)p.m(C)p.[fl2r](A)p.m(C)p.[thpyr](T)p.m(G)p.m(U)p.m(U)[Ssp].m(U) | ACACTGUUUU | |
| | [Ssp].m(U)}$$$$V2.0 | | |
| SSR-0106535 | RNA1{m(U)[Ssp].[fl2r](U)[Rsp].m(A)[n001S].[fl2r](U)p.m(A)p.[fl2r](G)p.m(A)p.m(G)p.m(C)p.m(A)[n001S | UUAUAGAGCAAGA | |
| | ].[fl2r](A)p.m(G)p.m(A)p.[fl2r](A)p.m(C)p.[fl2r](A)p.m(C)p.m(U)p.[thpyr](G)p.m(U)p.m(U)[Ssp].m(U) | ACACUGUUUU | |
| | [Ssp].m(U)}$$$$V2.0 | | |
| SSR-0106536 | RNA1{m(U)[Ssp].[fl2r](U)[Rsp].m(A)[n001S].[fl2r](U)p.m(A)p.[fl2r](G)p.m(A)p.m(G)p.m(C)p.m(A)[n001S | UUAUAGAGCAAGA | |
| | ].[fl2r](A)p.m(G)p.m(A)p.[fl2r](A)p.m(C)p.[fl2r](A)p.m(C)p.m(U)p.m(G)p.[thpyr](T)p.m(U)[Ssp].m(U) | ACACUGTUUU | |
| | [Ssp].m(U)}$$$$V2.0 | | |
| SSR-0106537 | RNA1{m(U)[Ssp].[fl2r](U)[Rsp].m(A)[n001S].[fl2r](U)p.m(A)p.[fl2r](G)p.m(A)p.m(G)p.m(C)p.m(A)[n001S | UUAUAGAGCAAGA | |
| | ].[fl2r](A)p.m(G)p.m(A)p.[fl2r](A)p.m(C)p.[fl2r](A)p.m(C)p.m(U)p.m(G)p.m(U)p.[thpyr](T)[Ssp].m(U) | ACACUGUTUU | |
| | [Ssp].m(U)}$$$$V2.0 | | |
| SSR-0106538 | RNA1{m(U)[Ssp].[fl2r](U)[Rsp].m(A)[n001S].[fl2r](U)p.m(A)p.[fl2r](G)p.m(A)p.m(G)p.m(C)p.m(A)[n001S | UUAUAGAGCAAGA | |
| | ].[fl2r](A)p.m(G)p.m(A)p.[fl2r](A)p.m(C)p.[fl2r](A)p.m(C)p.m(U)p.m(G)p.m(U)p.m(U)[Ssp].[thpyr](T) | ACACUGUUTU | |
| | [Ssp].m(U)}$$$$V2.0 | | |
| SSR-0106539 | RNA1{m(U)[Ssp].[fl2r](U)[Rsp].m(A)[n001S].[fl2r](U)p.m(A)p.[fl2r](G)p.m(A)p.m(G)p.m(C)p.m(A)[n001S | UUAUAGAGCAAGA | |
| | ].[fl2r](A)p.m(G)p.m(A)p.[fl2r](A)p.m(C)p.[fl2r](A)p.m(C)p.m(U)p.m(G)p.m(U)p.m(U)[Ssp].m(U)[Ssp]. | ACACUGUUUT | |
| | [thpyr](T)}$$$$V2.0 | | |
| SSR-0106584 | RNA1{m(U)[sp].[fl2r](C)[sp].m(U)[n001].[fl2r](A)p.m(C)p.[fl2r](U)p.m(U)[n001].[fl2r](G)p.m(U)p. | UCUACUUGUGUUA | |
| | [fl2r](G)p.m(U)p.m(U)p.m(A)[sp].[fl2r](C)[sp].m(A)[sp].[fl2r](G)[sp].m(C)[sp].[fl2r](A)[n001]. | CAGCACAGUU | |
| | m(C)[sp].[fl2r](A)[sp].m(G)[sp].m(U)[sp].m(U)}$$$$V2.0 | | |
| SSR-0106587 | RNA1{p.[Rm5d5m](U)[Rsp].[fl2r](C)[Rsp].m(C)[n001S].[fl2r](U)p.m(U)p.[fl2r](C)p.m(C)[n001S].[fl2r] | UCCUUCCCUGAAG | |
| | (C)p.m(U)p.[fl2r](G)p.m(A)p.m(A)p.m(G)[Ssp].[fl2r](G)[Ssp].m(U)[Ssp].[fl2r](U)[Ssp].m(C)[Ssp].[fl2r] | GUUCCUCCUU | |
| | (C)[n001S].m(U)[Ssp].[fl2r](C)[Ssp].m(C)[Ssp].m(U)[Ssp].m(U)}$$$$V2.0 | | |
| SSR-0106620 | RNA1{p.[Rm5d5m](U)[Rsp].[fl2r](U)[Rsp].m(A)[n001S].[fl2r](U)p.m(A)p.[fl2r](G)p.m(A)p.m(G)p.m(C)p. | UUAUAGAGCAAGA | |
| | m(A)[n001R].[fl2r](A)p.m(G)p.m(A)p.[fl2r](A)p.m(C)p.[fl2r](A)p.m(C)p.m(U)p.m(G)p.m(U)p.m(U)[Ssp].m | ACACUGUUUU | |
| | (U)[Ssp].m(U)}$$$$V2.0 | | |
| SSR-0106621 | RNA1{p.[Rm5d5m](U)[Ssp].[fl2r](U)[Ssp].m(A)[n001S].[fl2r](U)p.m(A)p.[fl2r](G)p.m(A)p.m(G)p.m(C)p.m | UUAUAGAGCAAGA | |
| | (A)[n001R].[fl2r](A)p.m(G)p.m(A)p.[fl2r](A)p.m(C)p.[fl2r](A)p.m(C)p.m(U)p.m(G)p.m(U)p.m(U)[Ssp].m( | ACACUGUUUU | |
| | U)[Ssp].m(U)}$$$$V2.0 | | |
| SSR-0106625 | RNA1{m(U)[Ssp].[fl2r](U)[Rsp].[thpyr](A)p.[fl2r](U)p.m(A)p.[fl2r](G)p.m(A)p.m(G)p.m(C)p.m(A)p.[fl2r] | UUAUAGAGCAAGA | |
| | (A)p.m(G)p.m(A)p.[fl2r](A)p.m(C)p.[fl2r](A)p.m(C)p.m(U)p.m(G)p.m(U)p.m(U)[Ssp].m(U)[Ssp].m(U)}$$$$ | ACACUGUUUU | |
| | V2.0 | | |
| SSR-0106626 | RNA1{m(U)[Ssp].[fl2r](U)[Rsp].m(A)p.[thpyr](T)p.m(A)p.[fl2r](G)p.m(A)p.m(G)p.m(C)p.m(A)p.[fl2r](A)p. | UUATAGAGCAAGA | |
| | m(G)p.m(A)p.[fl2r](A)p.m(C)p.[fl2r](A)p.m(C)p.m(U)p.m(G)p.m(U)p.m(U)[Ssp].m(U)[Ssp].m(U)}$$$$V2.0 | ACACUGUUUU | |
| SSR-0106627 | RNA1{m(U)[Ssp].[fl2r](U)[Rsp].m(A)p.[fl2r](U)p.m(A)p.[fl2r](G)p.m(A)p.m(G)p.m(C)p.[thpyr](A)p.[fl2r] | UUAUAGAGCAAGA | |
| | (A)p.m(G)p.m(A)p.[fl2r](A)p.m(C)p.[fl2r](A)p.m(C)p.m(U)p.m(G)p.m(U)p.m(U)[Ssp].m(U)[Ssp].m(U)}$$$$ | ACACUGUUUU | |
| | V2.0 | | |
| SSR-0106628 | RNA1{m(U)[Ssp].[fl2r](U)[Rsp].m(A)p.[fl2r](U)p.m(A)p.[fl2r](G)p.m(A)p.m(G)p.m(C)p.m(A)p.[thpyr](A)p. | UUAUAGAGCAAGA | |
| | m(G)p.m(A)p.[fl2r](A)p.m(C)p.[fl2r](A)p.m(C)p.m(U)p.m(G)p.m(U)p.m(U)[Ssp].m(U)[Ssp].m(U)}$$$$V2.0 | ACACUGUUUU | |
| SSR-0107427 | RNA1{p.[Rm5d5m](U)[Rsp].[fl2r](U)[Ssp].m(U)[n001S].[fl2r](A)p.m(G)p.[fl2r](A)p.m(G)[n001S].[fl2r](U) | UUUAGAGUGAGG | |
| | p.[fl2r](G)p.m(A)p.m(G)p.m(G)p.m(A)[Ssp].[fl2r](U)[Ssp].m(U)[Ssp].[fl2r](A)[Ssp].m(A)[Ssp].m(A)[n001S] | AUUAAAAUGUU | |
| | .m(A)[Ssp].m(U)[Ssp].m(G)[Ssp].m(U)[Ssp].m(U)}$$$$V2.0 | | |
| SSR-0107428 | RNA1{p.[Rm5d5m](U)[Rsp].[fl2r](U)[Ssp].m(U)[n001S].[fl2r](A)p.m(G)p.[fl2r](A)p.m(G)[n001S].[fl2r](U) | UUUAGAGUGAGG | |
| | p.[fl2r](G)p.m(A)p.m(G)p.m(G)p.m(A)p.[fl2r](U)p.m(U)p.[fl2r](A)p.m(A)p.m(A)[n001S].m(A)p.m(U)p.m( | AUUAAAAUGUU | |
| | G}[Ssp].m(U)[Ssp].m(U)}$$$$V2.0 | | |
| SSR-0108082 | RNA1{p.[Rm5d5m](U)[Rsp].[fl2r](A)[Ssp].m(G)[n001S].[fl2r](A)p.m(C)p.[fl2r](A)p.m(A)[n001S].[fl2r](U)p | UAGACAAUAAAUA | |
| | .[fl2r](A)p.m(A)p.m(A)p.m(U)p.m(A)[Ssp].[fl2r](C)[Ssp].m(C)[Ssp].[fl2r](G)[Ssp].m(A)[Ssp].m(G)[n001S]. | CCGAGGAAUU | |
| | m(G)[Ssp].m(A)[Ssp].m(A)[Ssp].m(U)[Ssp].m(U)}$$$$V2.0 | | |
| SSR-0108084 | RNA1{p.[Rm5d5m](U)[Rsp].[fl2r](A)[Ssp].m(G)[n001S].[fl2r](A)p.m(C)p.[fl2r](A)p.m(A)[n001S].[fl2r](U)p | UAGACAAUAAAUA | |
| | .[fl2r](A)p.m(A)p.m(A)p.m(U)p.m(A)p.[fl2r](C)p.m(C)p.[fl2r](G)p.m(A)p.m(G)[n001S].m(G)p.m(A)p.m(A) | CCGAGGAAUU | |
| | [Ssp].m(U)[Ssp].m(U)}$$$$V2.0 | | |
| SSR-0108085 | RNA1{p.[Rm5d5m](U)[Rsp].[fl2r](A)[Ssp].m(G)[n001S].[fl2r](A)p.m(C)p.[fl2r](A)p.m(A)[n001S].[fl2r] | UAGACAAUAAAUA | |
| | (U)p.[fl2r](A)p.m(A)p.m(A)p.m(U)p.m(A)p.[fl2r](C)p.m(C)p.[fl2r](G)p.m(A)p.m(G)p.m(G)p.m(A)p.m(A) | CCGAGGAAUU | |
| | [Ssp].m(U)[Ssp].m(U)}$$$$V2.0 | | |

TABLE le
Example Passenger Strand Compositions.

| ID | HELM | BASE_SEQUENCE | NAME |
|---|---|---|---|
| SSR-0101596 | RNA1{p.m(A)[sp].m(A)p.m(C)p.m(A)p.m(G)p.m(U)p.[fl2r](G)p.m(U)p.[fl2r](U)p.[fl2r](C)p.[fl2r](U)p. | AACAGUGUUCU | WV-41828 |
| | m(U)p.m(G)p.m(C)p.m(U)p.m(C)p.m(U)p.m(A)p.m(U)p.m(A)[sp].m(A)}\|CHEM1{[GalNAc3C12oyl]}\| | UGCUCUAUAA | |
| | CHEM2{[nC6o]}$CHEM2,RNA1,1:R1-1:R1\|CHEM2,CHEM1,1:R2-1:R1$$$V2.0 | | |
| SSR-0101599 | RNA1{p.m(A)[Ssp].m(A)p.m(C)p.m(A)p.m(G)p.m(U)p.[fl2r](G)p.m(U)p.[fl2r](U)p.[fl2r](C)p.[fl2r](U) | AACAGUGUUCU | WV-42080 |
| | p.m(U)p.m(G)p.m(C)p.m(U)p.m(C)p.m(U)p.m(A)p.m(U)p.m(A)[Ssp].m(A)}\|CHEM1{[GalNAc3C12oy | UGCUCUAUAA | |
| | l]]}\|CHEM2{[nC6o]}$CHEM2,RNA1,1:R1-1:R1\|CHEM2,CHEM1,1:R2-1:R1$$$V2.0 | | |
| SSR-0105234 | RNA1{p.m(G)[Ssp].m(G)p.m(A)p.m(G)p.m(G)p.m(A)p.[fl2r](A)p.m(C)p.[fl2r](C)p.[fl2r](U)p.[fl2r](U) | GGAGGAACCUUC | WV-49615 |
| | p.m(C)p.m(A)p.m(G)p.m(G)p.m(G)p.m(A)p.m(A)p.m(G)p.m(G)[Ssp].m(A)}\|CHEM1{[GalNAc3C12oy | AGGGAAGGA | |
| | l]]}\|CHEM2{[nC6o]}$CHEM2,RNA1,1:R1-1:R1\|CHEM2,CHEM1,1:R2-1:R1$$$V2.0 | | |
| SSR-0106206 | RNA1{m(C)[Ssp].m(U)p.[fl2r](G)p.m(U)p.[fl2r](G)p.m(C)p.[fl2r](U)[n001R].m(G)p.[fl2r](U)p.[fl2r](A) | CUGUGCUGUAA | |
| | p.[fl2r](A)p.m(C)p.[fl2r](A)p.m(C)p.[fl2r](A)[n001R].m(A)p.[fl2r](G)p.m(U)p.[fl2r](A)p.m(G)[Ssp]. | CACAAGUAGA | |
| | m(A)}$$$$V2.0 | | |
| SSR-0106503 | RNA1{m(C)[Ssp].m(U)p.[fl2r](G)p.m(U)p.[fl2r](G)p.m(C)p.[fl2r](U)[n001R].m(G)p.[fl2r](U)p.[fl2r](A) | CUGUGCUGUAA | |
| | p.[fl2r](A)p.m(C)p.[fl2r](A)p.m(C)p.[fl2r](A)p.m(A)p.[fl2r](G)p.m(U)p.[fl2r](A)p.m(G)[Ssp].[fl2r] | CACAAGUAGA | |
| | (A)}$$$$V2.0 | | |
| SSR-0106506 | RNA1{m(C)[Ssp].m(U)p.[fl2r](G)p.m(U)p.[fl2r](G)p.m(C)p.[hexdec2r](U)p.m(G)p.[fl2r](U)p.[fl2r](A) | CUGUGCUGUAA | |
| | p.[fl2r](A)p.m(C)p.[fl2r](A)p.m(C)p.[fl2r](A)p.m(A)p.[fl2r](G)p.m(U)p.[fl2r](A)p.m(G)[Ssp].[fl2r] | CACAAGUAGA | |
| | (A)}$$$$V2.0 | | |
| SSR-0106507 | RNA1{m(C)[Ssp].m(U)p.[fl2r](G)p.m(U)p.[fl2r](G)p.m(C)p.[hexdec2r](U)[n001R].m(G)p.[fl2r](U)p.[f | CUGUGCUGUAA | |
| | 12r](A)p.[fl2r](A)p.m(C)p.[fl2r](A)p.m(C)p.[fl2r](A)p.m(A)p.[fl2r](G)p.m(U)p.[fl2r](A)p.m(G)[Ssp]. | CACAAGUAGA | |
| | [fl2r](A)}$$$$V2.0 | | |
| SSR-0106508 | RNA1{m(C)[Ssp].m(U)p.[fl2r](G)p.m(U)p.[fl2r](G)p.m(C)p.[hexdec2r](U)p.m(G)p.[fl2r](U)p.[fl2r](A) | CUGUGCUGUAA | |
| | p.[fl2r](A)p.m(C)p.[fl2r](A)p.m(C)p.[fl2r](A)[n001R].m(A)p.[fl2r](G)p.m(U)p.[fl2r](A)p.m(G)[Ssp]. | CACAAGUAGA | |
| | [fl2r](A)}$$$$V2.0 | | |
| SSR-0106509 | RNA1{m(C)[Ssp].m(U)p.[fl2r](G)p.m(U)p.[fl2r](G)p.m(C)p.[hexdec2r](U)[n001R].m(G)p.[fl2r](U)p.[f | CUGUGCUGUAA | |
| | 12r](A)p.[fl2r](A)p.m(C)p.[fl2r](A)p.m(C)p.[fl2r](A)[n001R].m(A)p.[fl2r](G)p.m(U)p.[fl2r](A)p.m(G) | CACAAGUAGA | |
| | [Ssp].[fl2r](A)}$$$$V2.0 | | |
| SSR-0106510 | RNA1{m(C)[Ssp].m(U)[Ssp].[fl2r](G)[Ssp].m(U)[Ssp].[fl2r](G)[Ssp].m(C)[Ssp].[hexdec2r](U)[n001R]. | CUGUGCUGUAA | |
| | m(G)p.[fl2r](U)p.[fl2r](A)p.[fl2r](A)p.m(C)p.[fl2r](A)p.m(C)p.[fl2r](A)[n001R].m(A)[Ssp].[fl2r](G) | CACAAGUAGA | |
| | [Ssp].m(U)[Ssp].[fl2r](A)[Ssp].m(G)[Ssp].m(A)}$$$$V2.0 | | |
| SSR-0106511 | RNA1{m(C)[Ssp].m(U)p.[fl2r](G)p.m(U)p.[fl2r](G)p.m(C)p.[fl2r](U)[n029R].m(G)p.[fl2r](U)p.[fl2r](A) | CUGUGCUGUAA | |
| | p.[fl2r](A)p.m(C)p.[fl2r](A)p.m(C)p.[fl2r](A)[n029R].m(A)p.[fl2r](G)p.m(U)p.[fl2r](A)p.m(G)[Ssp]. | CACAAGUAGA | |
| | [fl2r](A)}$$$$V2.0 | | |
| SSR-0106513 | RNA1{m(C)[Ssp].m(U)[Ssp].[fl2r](G)[Ssp].m(U)[Ssp].[fl2r](G)[Ssp].m(C)[Ssp].[fl2r](U)[n029R].m(G)p | CUGUGCUGUAA | |
| | .[fl2r](U)p.[fl2r](A)p.[fl2r](A)p.m(C)p.[fl2r](A)p.m(C)p.[fl2r](A)[n029R].m(A)[Ssp].[fl2r](G)[Ssp]. | CACAAGUAGA | |
| | m(U)[Ssp].[fl2r](A)[Ssp].m(G)[Ssp].m(A)}$$$$V2.0 | | |
| SSR-0106563 | RNA1{m(C)[sp].m(U)p.[fl2r](G)p.m(U)p.[fl2r](G)p.m(C)p.[fl2r](U)[n001].m(G)p.[fl2r](U)p.[fl2r](A)p. | CUGUGCUGUAA | |
| | [fl2r](A)p.m(C)p.[fl2r](A)p.m(C)p.[fl2r](A)[n001].m(A)p.[fl2r](G)p.m(U)p.[fl2r](A)p.m(G)[sp].m(A)} | CACAAGUAGA | |
| | $$$$V2.0 | | |
| SSR-0106589 | RNA1{m(G)[Ssp].m(G)p.[fl2r](A)p.m(G)p.[fl2r](G)p.m(A)p.[fl2r](A)[n001R].m(C)p.[fl2r](C)p.[fl2r](U) | GGAGGAACCUUC | |
| | p.[fl2r](U)p.m(C)p.[fl2r](A)p.m(G)p.[fl2r](G)[n001R].m(G)p.[fl2r](A)p.m(A)p.[fl2r](G)p.m(G)[Ssp].m | AGGGAAGGA | |
| | (A)}$$$$V2.0 | | |
| SSR-0106597 | RNA1{m(C)[sp].m(U)p.[fl2r](G)p.m(U)p.[fl2r](G)p.m(C)p.[hexdec2r](U)p.m(G)p.[fl2r](U)p.[fl2r](A)p. | CUGUGCUGUAA | |
| | [fl2r](A)p.m(C)p.[fl2r](A)p.m(C)p.[fl2r](A)p.m(A)p.[fl2r](G)p.m(U)p.[fl2r](A)p.m(G)[sp].[fl2r](A)}$$$ | CACAAGUAGA | |
| | $V2.0 | | |
| SSR-0106598 | RNA1{m(C)[sp].m(U)p.[fl2r](G)p.m(U)p.[fl2r](G)p.m(C)p.[hexdec2r](U)[n001R].m(G)p.[fl2r](U)p.[fl | CUGUGCUGUAA | |
| | 2r](A)p.[fl2r](A)p.m(C)p.[fl2r](A)p.m(C)p.[fl2r](A)p.m(A)p.[fl2r](G)p.m(U)p.[fl2r](A)p.m(G)[sp].[fl2r] | CACAAGUAGA | |
| | (A)}$$$$V2.0 | | |
| SSR-0106599 | RNA1{m(C)[sp].m(U)p.[fl2r](G)p.m(U)p.[fl2r](G)p.m(C)p.[hexdec2r](U)[n009R].m(G)p.[fl2r](U)p.[fl | CUGUGCUGUAA | |
| | 2r](A)p.[fl2r](A)p.m(C)p.[fl2r](A)p.m(C)p.[fl2r](A)p.m(A)p.[fl2r](G)p.m(U)p.[fl2r](A)p.m(G)[sp].[fl2r] | CACAAGUAGA | |
| | (A)}$$$$V2.0 | | |
| SSR-0106600 | RNA1{m(C)[sp].m(U)p.[fl2r](G)p.m(U)p.[fl2r](G)p.m(C)p.[hexdec2r](U)[n033R].m(G)p.[fl2r](U)p.[fl | CUGUGCUGUAA | |
| | 2r](A)p.[fl2r](A)p.m(C)p.[fl2r](A)p.m(C)p.[fl2r](A)p.m(A)p.[fl2r](G)p.m(U)p.[fl2r](A)p.m(G)[sp].[fl2r] | CACAAGUAGA | |
| | (A)}$$$$V2.0 | | |
| SSR-0106601 | RNA1{m(C)[Ssp].m(U)p.[fl2r](G)p.m(U)p.[fl2r](G)p.m(C)p.[hexdec2r](U)[n009R].m(G)p.[fl2r](U)p.[f | CUGUGCUGUAA | |
| | 12r](A)p.[fl2r](A)p.m(C)p.[fl2r](A)p.m(C)p.[fl2r](A)p.m(A)p.[fl2r](G)p.m(U)p.[fl2r](A)p.m(G)[Ssp].[fl2 | CACAAGUAGA | |
| | r](A)}$$$$V2.0 | | |
| SSR-0106602 | RNA1{m(C)[Ssp].m(U)p.[fl2r](G)p.m(U)p.[fl2r](G)p.m(C)p.[hexdec2r](U)[n033R].m(G)p.[fl2r](U)p.[f | CUGUGCUGUAA | |
| | 12r](A)p.[fl2r](A)p.m(C)p.[fl2r](A)p.m(C)p.[fl2r](A)p.m(A)p.[fl2r](G)p.m(U)p.[fl2r](A)p.m(G)[Ssp].[fl2 | CACAAGUAGA | |
| | r](A)}$$$$V2.0 | | |
| SSR-0106603 | RNA1{m(C)[Ssp].m(U)p.[fl2r](G)p.m(U)p.[fl2r](G)p.m(C)p.[fl2r](U)[n031R].m(G)p.[fl2r](U)p.[fl2r](A) | CUGUGCUGUAA | |
| | p.[fl2r](A)p.m(C)p.[fl2r](A)p.m(C)p.[fl2r](A)p.m(A)p.[fl2r](G)p.m(U)p.[fl2r](A)p.m(G)[Ssp].[fl2r](A)} | CACAAGUAGA | |
| | $$$$V2.0 | | |
| SSR-0106604 | RNA1{m(C)[Ssp].m(U)p.[fl2r](G)p.m(U)p.[fl2r](G)p.m(C)p.[fl2r](U)[n037R].m(G)p.[fl2r](U)p.[fl2r](A) | CUGUGCUGUAA | |
| | p.[fl2r](A)p.m(C)p.[fl2r](A)p.m(C)p.[fl2r](A)p.m(A)p.[fl2r](G)p.m(U)p.[fl2r](A)p.m(G)[Ssp].[fl2r](A)} | CACAAGUAGA | |
| | $$$$V2.0 | | |
| SSR-0106605 | RNA1{m(C)[Ssp].m(U)p.[fl2r](G)p.m(U)p.[fl2r](G)p.m(C)p.[fl2r](U)[n046R].m(G)p.[fl2r](U)p.[fl2r](A) | CUGUGCUGUAA | |
| | p.[fl2r](A)p.m(C)p.[fl2r](A)p.m(C)p.[fl2r](A)p.m(A)p.[fl2r](G)p.m(U)p.[fl2r](A)p.m(G)[Ssp].[fl2r](A)} | CACAAGUAGA | |
| | $$$$V2.0 | | |
| SSR-0106606 | RNA1{m(C)[Ssp].m(U)p.[fl2r](G)p.m(U)p.[fl2r](G)p.m(C)p.[fl2r](U)[n047R].m(G)p.[fl2r](U)p.[fl2r](A) | CUGUGCUGUAA | |
| | p.[fl2r](A)p.m(C)p.[fl2r](A)p.m(C)p.[fl2r](A)p.m(A)p.[fl2r](G)p.m(U)p.[fl2r](A)p.m(G)[Ssp].[fl2r](A)} | CACAAGUAGA | |
| | $$$$V2.0 | | |
| SSR-0106607 | RNA1{m(C)[Ssp].m(U)p.[fl2r](G)p.m(U)p.[fl2r](G)p.m(C)p.[fl2r](U)[n043R].m(G)p.[fl2r](U)p.[fl2r](A) | CUGUGCUGUAA | |
| | p.[fl2r](A)p.m(C)p.[fl2r](A)p.m(C)p.[fl2r](A)p.m(A)p.[fl2r](G)p.m(U)p.[fl2r](A)p.m(G)[Ssp].[fl2r](A)} | CACAAGUAGA | |
| | $$$$V2.0 | | |
| SSR-0106629 | RNA1{p.m(A)[Ssp].m(A)p.m(C)p.m(A)p.m(G)p.m(U)p.[fl2r](G)p.m(U)p.[fl2r](U)p.[fl2r](C)p.[fl2r](U) | AACAGUGUUCU | |
| | p.m(U)p.m(G)p.m(C)p.m(U)p.m(C)p.m(U)p.m(A)p.m(U)p.m(A)[Ssp].[thpyr](A)}\|CHEM1{[nC6o]}\|C | UGCUCUAUAA | |
| | HEM2{[GalNAc3C12oyl]}$CHEM1,RNA1,1:R1-1:R1\|CHEM1,CHEM2,1:R2-1:R1$$$V2.0 | | |
| SSR-0106630 | RNA1{p.m(A)[Ssp].m(A)p.m(C)p.m(A)p.m(G)p.m(U)p.[fl2r](G)p.m(U)p.[fl2r](U)p.[fl2r](C)p.[fl2r](U) | AACAGUGUUCU | |
| | p.m(U)p.m(G)p.m(C)p.m(U)p.m(C)p.m(U)p.[thpyr](A)p.m(U)p.m(A)[Ssp].m(A)}\|CHEM1{[nC6o]}\|C | UGCUCUAUAA | |
| | HEM2{[GalNAc3C12oyl]}$CHEM1,RNA1,1:R1-1:R1\|CHEM1,CHEM2,1:R2-1:R1$$$V2.0 | | |
| SSR-0106631 | RNA1{p.m(A)[Ssp].m(A)p.m(C)p.m(A)p.m(G)p.m(U)p.[fl2r](G)p.m(U)p.[fl2r](U)p.[fl2r](C)p.[fl2r](U) | AACAGUGUUCU | |
| | p.m(U)p.m(G)p.m(C)p.m(U)p.m(C)p.m(U)p.m(A)p.[thpyr](T)p.m(A)[Ssp].m(A)}\|CHEM1{[nC6o]}\|CH | UGCUCUATAA | |
| | EM2{[GalNAc3C12oyl]}$CHEM1,RNA1,1:R1-1:R1\|CHEM1,CHEM2,1:R2-1:R1$$$V2.0 | | |
| SSR-0106632 | RNA1{p.m(A)[Ssp].m(A)p.m(C)p.m(A)p.m(G)p.m(U)p.[fl2r](G)p.m(U)p.[fl2r](U)p.[fl2r](C)p.[fl2r](U) | AACAGUGUUCU | |
| | p.m(U)p.m(G)p.m(C)p.m(U)p.m(C)p.m(U)p.m(A)p.m(U)p.[thpyr](A)[Ssp].m(A)}\|CHEM1{[nC6o]}\|C | UGCUCUAUAA | |
| | HEM2{[GalNAc3C12oyl]}$CHEM1,RNA1,1:R1-1:R1\|CHEM1,CHEM2,1:R2-1:R1$$$V2.0 | | |
| SSR-0106633 | RNA1{p.m(A)[Ssp].m(A)p.m(C)p.m(A)p.m(G)p.m(U)p.[fl2r](G)p.m(U)p.[fl2r](U)p.[fl2r](C)p.[fl2r](U) | AACAGUGUUCU | |
| | p.m(U)p.m(G)p.m(C)p.m(U)p.m(C)p.[thpyr](T)p.m(A)p.m(U)p.m(A)[Ssp].m(A)}\|CHEM1{[nC6o]}\|CH | UGCUCTAUAA | |
| | EM2{[GalNAc3C12oyl]}$CHEM1,RNA1,1:R1-1:R1\|CHEM1,CHEM2,1:R2-1:R1$$$V2.0 | | |
| SSR-0106634 | RNA1{p.m(A)[Ssp].m(A)p.m(C)p.m(A)p.m(G)p.m(U)p.[fl2r](G)p.m(U)p.[fl2r](U)p.[fl2r](C)p.[fl2r](U) | AACAGUGUUCU | |
| | p.m(U)p.m(G)p.[thpyr](C)p.m(U)p.m(C)p.m(U)p.m(A)p.m(U)p.m(A)[Ssp].m(A)}\|CHEM1{[nC6o]}\|C | UGCUCUAUAA | |
| | HEM2{[GalNAc3C12oyl]}$CHEM1,RNA1,1:R1-1:R1\|CHEM1,CHEM2,1:R2-1:R1$$$V2.0 | | |
| SSR-0106635 | RNA1{p.m(A)[Ssp].m(A)p.m(C)p.m(A)p.m(G)p.m(U)p.[fl2r](G)p.m(U)p.[fl2r](U)p.[fl2r](C)p.[fl2r](U) | AACAGUGUUCU | |
| | p.m(U)p.m(G)p.m(C)p.[thpyr](T)p.m(C)p.m(U)p.m(A)p.m(U)p.m(A)[Ssp].m(A)}\|CHEM1{[nC6o]}\|CH | UGCTCUAUAA | |
| | EM2{[GalNAc3C12oyl]}$CHEM1,RNA1,1:R1-1:R1\|CHEM1,CHEM2,1:R2-1:R1$$$V2.0 | | |
| SSR-0106637 | RNA1{p.m(A)[Ssp].m(A)p.m(C)p.m(A)p.m(G)p.m(U)p.[fl2r](G)p.m(U)p.[fl2r](U)p.[fl2r](C)p.[fl2r](U) | AACAGUGUUCU | |
| | p.m(U)p.[thpyr](G)p.m(C)p.m(U)p.m(C)p.m(U)p.m(A)p.m(U)p.m(A)[Ssp].m(A)}\|CHEM1{[nC6o]}\|C | UGCUCUAUAA | |
| | HEM2{[GalNAc3C12oyl]}$CHEM1,RNA1,1:R1-1:R1\|CHEM1,CHEM2,1:R2-1:R1$$$V2.0 | | |
| SSR-0106638 | RNA1{p.m(A)[Ssp].m(A)p.m(C)p.m(A)p.m(G)p.m(U)p.[fl2r](G)p.m(U)p.[fl2r](U)p.[fl2r](C)p.[fl2r](U) | AACAGUGUUCUT | |
| | p.[thpyr](T)p.m(G)p.m(C)p.m(U)p.m(C)p.m(U)p.m(A)p.m(U)p.m(A)[Ssp].m(A)}\|CHEM1{[nC6o]}\|CH | GCUCUAUAA | |
| | EM2{[GalNAc3C12oyl]}$CHEM1,RNA1,1:R1-1:R1\|CHEM1,CHEM2,1:R2-1:R1$$$V2.0 | | |
| SSR-0106640 | RNA1{p.m(A)[Ssp].m(A)p.m(C)p.m(A)p.m(G)p.m(U)p.[fl2r](G)p.m(U)p.[fl2r](U)p.[fl2r](C)p.[fl2r](U) | AACAGUGUUCU | |
| | p.m(U)p.m(G)p.m(C)p.m(U)p.[thpyr](C)p.m(U)p.m(A)p.m(U)p.m(A)[Ssp].m(A)}\|CHEM1{[nC6o]}\|C | UGCUCUAUAA | |
| | HEM2{[GalNAc3C12oyl]}$CHEM1,RNA1,1:R1-1:R1\|CHEM1,CHEM2,1:R2-1:R1$$$V2.0 | | |
| SSR-0106643 | RNA1{p.m(A)[sp].m(A)p.m(C)p.m(A)p.m(G)p.m(U)p.[fl2r](G)p.m(U)p.[fl2r](U)p.[fl2r](C)p.[fl2r](U)p. | AACAGUGUUCU | |
| | m(U)p.m(G)p.m(C)p.m(U)p.m(C)p.m(U)p.m(A)p.m(U)p.[thpyr](A)[sp].m(A)}\|CHEM1{[nC6o]}\|CHE | UGCUCUAUAA | |
| | M2{[GalNAc3C12oyl]}$CHEM1,RNA1,1:R1-1:R1\|CHEM1,CHEM2,1:R2-1:R1$$$V2.0 | | |
| SSR-0107400 | RNA1{m(C)[Ssp].m(A)p.m(U)p.m(U)p.m(U)p.m(U)p.[fl2r](A)[n001R].m(A)p.[fl2r](U)p.[fl2r](C)p.[fl2r | CAUUUUAAUCCU | |
| | ](C)p.m(U)p.m(C)p.m(A)p.m(C)[n001R].m(U)p.m(C)p.m(U)p.m(A)p.m(A)[Ssp].m(A)}$$$$V2.0 | CACUCUAAA | |
| SSR-0107412 | RNA1{m(C)[Ssp].m(A)p.m(U)p.m(U)p.m(U)p.m(U)p.[fl2r](A)[n009R].m(A)p.[fl2r](U)p.[fl2r](C)p.[fl2r | CAUUUUAAUCCU | |
| | ](C)p.m(U)p.m(C)p.m(A)p.m(C)[n009R].m(U)p.m(C)p.m(U)p.m(A)p.m(A)[Ssp].m(A)}$$$$V2.0 | CACUCUAAA | |
| SSR-0107413 | RNA1{m(C)[Ssp].m(A)p.m(U)p.m(U)p.m(U)p.m(U)p.[fl2r](A)[n001R].m(A)p.[fl2r](U)p.[fl2r](C)p.[fl2r | CAUUUUAAUCCU | |
| | (C)p.m(U)p.m(C)p.m(A)p.m(C)p.m(U)p.m(C)p.m(U)p.m(A)p.m(A)[Ssp].m(A)}$$$$V2.0 | CACUCUAAA | |
| SSR-0107414 | RNA1{m(C)[Ssp].m(A)p.m(U)p.m(U)p.m(U)p.m(U)p.[fl2r](A)[n009R].m(A)p.[fl2r](U)p.[fl2r](C)p.[fl2r | CAUUUUAAUCCU | |
| | ](C)p.m(U)p.m(C)p.m(A)p.m(C)p.m(U)p.m(C)p.m(U)p.m(A)p.m(A)[Ssp].m(A)}$$$$V2.0 | CACUCUAAA | |
| SSR-0107415 | RNA1{m(C)[Ssp].m(A)p.m(U)p.m(U)p.m(U)p.m(U)p.[fl2r](A)[n039R].m(A)p.[fl2r](U)p.[fl2r](C)p.[fl2r | CACUCUAAA | |
| | ](C)p.m(U)p.m(C)p.m(A)p.m(C)p.m(U)p.m(C)p.m(U)p.m(A)p.m(A)[Ssp].m(A)}$$$$V2.0 | CAUUUUAAUCCU | |
| SSR-0107419 | RNA1{m(C)[Ssp].m(A)p.m(U)p.m(U)p.m(U)p.m(U)p.[fl2r](A)[n009R].m(A)p.[fl2r](U)p.[fl2r](C)p.[fl2r | CAUUUUAAUCCU | |
| | ](C)p.m(U)p.m(C)p.m(A)p.m(C)[n001R].m(U)p.m(C)p.m(U)p.m(A)p.m(A)[Ssp].m(A)}$$$$V2.0 | CACUCUAAA | |
| SSR-0107420 | RNA1{m(C)[Ssp].m(A)p.m(U)p.m(U)p.m(U)p.m(U)p.[fl2r](A)[n033R].m(A)p.[fl2r](U)p.[fl2r](C)p.[fl2r | CAUUUUAAUCCU | |
| | ](C)p.m(U)p.m(C)p.m(A)p.m(C)[n001R].m(U)p.m(C)p.m(U)p.m(A)p.m(A)[Ssp].m(A)}$$$$V2.0 | CACUCUAAA | |
| SSR-0107421 | RNA1{m(C)[Ssp].m(A)p.m(U)p.m(U)p.m(U)p.m(U)p.[fl2r](A)[n033R].m(A)p.[fl2r](U)p.[fl2r](C)p.[fl2r | CAUUUUAAUCCU | |
| | ](C)p.m(U)p.m(C)p.m(A)p.m(C)p.m(U)p.m(C)p.m(U)p.m(A)p.m(A)[Ssp].m(A)}$$$$V2.0 | CACUCUAAA | |
| SSR-0108086 | RNA1{m(U)[Ssp].m(U)p.m(C)p.m(C)p.m(U)p.m(C)p.[fl2r](G)[n009R].m(G)p.[fl2r](U)p.[fl2r](A)p.[fl2r | UUCCUCGGUAU | |
| | ](U)p.m(U)p.m(U)p.m(A)p.m(U)[n009R].m(U)p.m(G)p.m(U)p.m(C)p.m(U)[Ssp].m(A)}$$$$V2.0 | UUAUUGUCUA | |

TABLE 1f
Example Oligonucleotides/Compositions

| Duplex | Guide | Passenger |
|---|---|---|
| DSR-0101651 | SSR-0104720 | SSR-0101596 |
| DSR-0102161 | SSR-0104474 | SSR-0101599 |
| DSR-0102524 | SSR-0104475 | SSR-0101599 |
| DSR-0102630 | SSR-0104036 | SSR-0104097 |
| DSR-0102631 | SSR-0104066 | SSR-0104097 |
| DSR-0102632 | SSR-0104052 | SSR-0104021 |
| DSR-0102633 | SSR-0104082 | SSR-0104021 |
| DSR-0102634 | SSR-0104031 | SSR-0104099 |
| DSR-0102635 | SSR-0104061 | SSR-0104099 |
| DSR-0102636 | SSR-0104037 | SSR-0104026 |
| DSR-0102637 | SSR-0104067 | SSR-0104026 |
| DSR-0102638 | SSR-0104035 | SSR-0104030 |
| DSR-0102639 | SSR-0104065 | SSR-0104030 |
| DSR-0102640 | SSR-0104043 | SSR-0104092 |
| DSR-0102641 | SSR-0104073 | SSR-0104092 |
| DSR-0102661 | SSR-0104054 | SSR-0104022 |
| DSR-0102662 | SSR-0104084 | SSR-0104022 |
| DSR-0102663 | SSR-0104053 | SSR-0104022 |
| DSR-0102664 | SSR-0104083 | SSR-0104022 |
| DSR-0102665 | SSR-0104039 | SSR-0104027 |
| DSR-0102666 | SSR-0104069 | SSR-0104027 |
| DSR-0102667 | SSR-0104038 | SSR-0104027 |
| DSR-0102668 | SSR-0104068 | SSR-0104027 |
| DSR-0102669 | SSR-0104045 | SSR-0104094 |
| DSR-0102670 | SSR-0104075 | SSR-0104094 |
| DSR-0102671 | SSR-0104044 | SSR-0104094 |
| DSR-0102672 | SSR-0104074 | SSR-0104094 |
| DSR-0103133 | SSR-0106188 | SSR-0104027 |
| DSR-0103134 | SSR-0106190 | SSR-0104027 |
| DSR-0103135 | SSR-0106180 | SSR-0104027 |
| DSR-0103136 | SSR-0106181 | SSR-0104027 |
| DSR-0103137 | SSR-0104101 | SSR-0104029 |
| DSR-0103138 | SSR-0106185 | SSR-0104026 |
| DSR-0103139 | SSR-0106189 | SSR-0104026 |
| DSR-0103140 | SSR-0106195 | SSR-0104026 |
| DSR-0103141 | SSR-0106172 | SSR-0104026 |
| DSR-0103142 | SSR-0106184 | SSR-0104026 |
| DSR-0103143 | SSR-0106189 | SSR-0104027 |
| DSR-0103144 | SSR-0106170 | SSR-0104027 |
| DSR-0103145 | SSR-0106172 | SSR-0104027 |
| DSR-0103146 | SSR-0106178 | SSR-0104027 |
| DSR-0103147 | SSR-0106194 | SSR-0104026 |
| DSR-0103148 | SSR-0106178 | SSR-0104026 |
| DSR-0103149 | SSR-0106181 | SSR-0104026 |
| DSR-0103150 | SSR-0106187 | SSR-0104027 |
| DSR-0103151 | SSR-0106197 | SSR-0104027 |
| DSR-0103152 | SSR-0106174 | SSR-0104027 |
| DSR-0103153 | SSR-0106191 | SSR-0104026 |
| DSR-0103154 | SSR-0106179 | SSR-0104026 |
| DSR-0103155 | SSR-0106182 | SSR-0104026 |
| DSR-0103156 | SSR-0106186 | SSR-0104027 |
| DSR-0103157 | SSR-0106173 | SSR-0104027 |
| DSR-0103158 | SSR-0106176 | SSR-0104027 |
| DSR-0103159 | SSR-0106184 | SSR-0104027 |
| DSR-0103160 | SSR-0106187 | SSR-0104026 |
| DSR-0103161 | SSR-0106188 | SSR-0104026 |
| DSR-0103162 | SSR-0106176 | SSR-0104026 |
| DSR-0103163 | SSR-0106177 | SSR-0104026 |
| DSR-0103164 | SSR-0106183 | SSR-0104026 |
| DSR-0103165 | SSR-0106185 | SSR-0104027 |
| DSR-0103166 | SSR-0106193 | SSR-0104027 |
| DSR-0103167 | SSR-0106175 | SSR-0104027 |
| DSR-0103168 | SSR-0106186 | SSR-0104026 |
| DSR-0103169 | SSR-0106190 | SSR-0104026 |
| DSR-0103170 | SSR-0106192 | SSR-0104026 |
| DSR-0103171 | SSR-0106175 | SSR-0104026 |
| DSR-0103172 | SSR-0104039 | SSR-0104026 |
| DSR-0103173 | SSR-0106180 | SSR-0104026 |
| DSR-0103174 | SSR-0106192 | SSR-0104027 |
| DSR-0103175 | SSR-0106195 | SSR-0104027 |
| DSR-0103176 | SSR-0106171 | SSR-0104027 |
| DSR-0103177 | SSR-0106179 | SSR-0104027 |
| DSR-0103178 | SSR-0106197 | SSR-0104026 |
| DSR-0103179 | SSR-0106171 | SSR-0104026 |
| DSR-0103180 | SSR-0106174 | SSR-0104026 |
| DSR-0103181 | SSR-0106194 | SSR-0104027 |
| DSR-0103182 | SSR-0106196 | SSR-0104027 |
| DSR-0103183 | SSR-0106182 | SSR-0104027 |
| DSR-0103184 | SSR-0106193 | SSR-0104026 |
| DSR-0103185 | SSR-0106170 | SSR-0104026 |
| DSR-0103186 | SSR-0106191 | SSR-0104027 |
| DSR-0103187 | SSR-0106177 | SSR-0104027 |
| DSR-0103188 | SSR-0106183 | SSR-0104027 |
| DSR-0103189 | SSR-0104037 | SSR-0104027 |
| DSR-0103190 | SSR-0104100 | SSR-0104028 |
| DSR-0103191 | SSR-0106196 | SSR-0104026 |
| DSR-0103192 | SSR-0106173 | SSR-0104026 |
| DSR-0103199 | SSR-0106222 | SSR-0104026 |
| DSR-0103200 | SSR-0106225 | SSR-0104026 |
| DSR-0103201 | SSR-0106188 | SSR-0106208 |
| DSR-0103202 | SSR-0106188 | SSR-0106210 |
| DSR-0103203 | SSR-0106188 | SSR-0106212 |
| DSR-0103204 | SSR-0106188 | SSR-0106218 |
| DSR-0103205 | SSR-0106224 | SSR-0104026 |
| DSR-0103206 | SSR-0106183 | SSR-0106447 |
| DSR-0103207 | SSR-0106188 | SSR-0106211 |
| DSR-0103208 | SSR-0106188 | SSR-0106215 |
| DSR-0103209 | SSR-0106188 | SSR-0106217 |
| DSR-0103210 | SSR-0104100 | SSR-0106445 |
| DSR-0103211 | SSR-0104100 | SSR-0106446 |
| DSR-0103212 | SSR-0106188 | SSR-0106207 |
| DSR-0103213 | SSR-0106188 | SSR-0106213 |
| DSR-0103214 | SSR-0106188 | SSR-0106214 |
| DSR-0103215 | SSR-0106188 | SSR-0106219 |
| DSR-0103216 | SSR-0106223 | SSR-0104026 |
| DSR-0103217 | SSR-0106183 | SSR-0106448 |
| DSR-0103218 | SSR-0106183 | SSR-0106452 |
| DSR-0103219 | SSR-0106188 | SSR-0106206 |
| DSR-0103220 | SSR-0106188 | SSR-0106216 |
| DSR-0103221 | SSR-0106220 | SSR-0104026 |
| DSR-0103222 | SSR-0106221 | SSR-0104026 |
| DSR-0103223 | SSR-0106183 | SSR-0106449 |
| DSR-0103224 | SSR-0106183 | SSR-0106450 |
| DSR-0103225 | SSR-0106183 | SSR-0106451 |
| DSR-0103226 | SSR-0106188 | SSR-0106209 |
| DSR-0103227 | SSR-0106266 | SSR-0101599 |
| DSR-0103228 | SSR-0106267 | SSR-0101599 |
| DSR-0103229 | SSR-0106268 | SSR-0101599 |
| DSR-0103230 | SSR-0106269 | SSR-0101599 |
| DSR-0103231 | SSR-0106270 | SSR-0101599 |
| DSR-0103232 | SSR-0106271 | SSR-0101599 |
| DSR-0103233 | SSR-0106272 | SSR-0101599 |
| DSR-0103234 | SSR-0106273 | SSR-0101599 |
| DSR-0103235 | SSR-0106274 | SSR-0101599 |
| DSR-0103236 | SSR-0106275 | SSR-0101599 |
| DSR-0103237 | SSR-0106276 | SSR-0101599 |
| DSR-0103238 | SSR-0106277 | SSR-0101599 |
| DSR-0103239 | SSR-0106278 | SSR-0101599 |
| DSR-0103240 | SSR-0106279 | SSR-0101599 |
| DSR-0103241 | SSR-0106280 | SSR-0101599 |
| DSR-0103242 | SSR-0106281 | SSR-0101599 |
| DSR-0103243 | SSR-0106282 | SSR-0101599 |
| DSR-0103244 | SSR-0106283 | SSR-0101599 |
| DSR-0103245 | SSR-0106284 | SSR-0101599 |
| DSR-0103246 | SSR-0106285 | SSR-0101599 |
| DSR-0103247 | SSR-0106286 | SSR-0101599 |
| DSR-0103248 | SSR-0106287 | SSR-0101599 |
| DSR-0103249 | SSR-0106288 | SSR-0101599 |
| DSR-0103250 | SSR-0106289 | SSR-0101599 |
| DSR-0103251 | SSR-0106290 | SSR-0101599 |
| DSR-0103252 | SSR-0106291 | SSR-0101599 |
| DSR-0103253 | SSR-0106292 | SSR-0101599 |
| DSR-0103254 | SSR-0106293 | SSR-0101599 |
| DSR-0103255 | SSR-0106294 | SSR-0101599 |
| DSR-0103256 | SSR-0106295 | SSR-0101599 |
| DSR-0103257 | SSR-0106296 | SSR-0101599 |
| DSR-0103258 | SSR-0106297 | SSR-0101599 |
| DSR-0103259 | SSR-0106298 | SSR-0101599 |
| DSR-0103260 | SSR-0106299 | SSR-0101599 |
| DSR-0103261 | SSR-0106300 | SSR-0101599 |
| DSR-0103262 | SSR-0106301 | SSR-0101599 |
| DSR-0103263 | SSR-0106302 | SSR-0101599 |
| DSR-0103264 | SSR-0106303 | SSR-0101599 |
| DSR-0103265 | SSR-0106304 | SSR-0101599 |
| DSR-0103266 | SSR-0106305 | SSR-0101599 |
| DSR-0103267 | SSR-0106306 | SSR-0101599 |
| DSR-0103268 | SSR-0106307 | SSR-0101599 |
| DSR-0103275 | SSR-0106183 | SSR-0106206 |
| DSR-0103276 | SSR-0106183 | SSR-0106209 |
| DSR-0103299 | SSR-0106222 | SSR-0106206 |
| DSR-0103300 | SSR-0106222 | SSR-0106209 |
| DSR-0103301 | SSR-0106222 | SSR-0106447 |
| DSR-0103319 | SSR-0106480 | SSR-0106206 |
| DSR-0103320 | SSR-0106481 | SSR-0106206 |
| DSR-0103321 | SSR-0106482 | SSR-0106206 |
| DSR-0103322 | SSR-0106483 | SSR-0106206 |
| DSR-0103323 | SSR-0106484 | SSR-0106206 |
| DSR-0103324 | SSR-0106487 | SSR-0106206 |
| DSR-0103325 | SSR-0104037 | SSR-0106206 |
| DSR-0103326 | SSR-0106479 | SSR-0106206 |
| DSR-0103337 | SSR-0104103 | SSR-0104107 |
| DSR-0103338 | SSR-0104104 | SSR-0104024 |
| DSR-0103339 | SSR-0104102 | SSR-0104106 |
| DSR-0103340 | SSR-0104105 | SSR-0104025 |
| DSR-0102626 | SSR-0104473 | SSR-0101599 |
| DSR-0102657 | SSR-0105217 | SSR-0105234 |
| DSR-0103028 | SSR-0104717 | SSR-0101596 |
| DSR-0103099 | SSR-0105245 | SSR-0101599 |
| DSR-0103327 | SSR-0106453 | SSR-0101599 |
| DSR-0103328 | SSR-0106454 | SSR-0101599 |
| DSR-0103329 | SSR-0106455 | SSR-0101599 |
| DSR-0103330 | SSR-0106456 | SSR-0101599 |
| DSR-0103331 | SSR-0106457 | SSR-0101599 |
| DSR-0103342 | SSR-0106584 | SSR-0106563 |
| DSR-0103343 | SSR-0104100 | SSR-0106563 |
| DSR-0103359 | SSR-0104186 | SSR-0101599 |
| DSR-0103360 | SSR-0106620 | SSR-0101599 |
| DSR-0103361 | SSR-0104189 | SSR-0101599 |
| DSR-0103362 | SSR-0106621 | SSR-0101599 |
| DSR-0103363 | SSR-0106530 | SSR-0101599 |
| DSR-0103364 | SSR-0104475 | SSR-0106630 |
| DSR-0103365 | SSR-0106527 | SSR-0101599 |
| DSR-0103366 | SSR-0106528 | SSR-0101599 |
| DSR-0103367 | SSR-0106533 | SSR-0101599 |
| DSR-0103368 | SSR-0106536 | SSR-0101599 |
| DSR-0103369 | SSR-0106625 | SSR-0101599 |
| DSR-0103370 | SSR-0106520 | SSR-0101596 |
| DSR-0103371 | SSR-0104475 | SSR-0106629 |
| DSR-0103372 | SSR-0106532 | SSR-0101599 |
| DSR-0103373 | SSR-0104475 | SSR-0106640 |
| DSR-0103374 | SSR-0104475 | SSR-0106637 |
| DSR-0103375 | SSR-0104475 | SSR-0106638 |
| DSR-0103376 | SSR-0106522 | SSR-0101599 |
| DSR-0103377 | SSR-0106523 | SSR-0101599 |
| DSR-0103378 | SSR-0106524 | SSR-0101599 |
| DSR-0103379 | SSR-0106526 | SSR-0101599 |
| DSR-0103380 | SSR-0106534 | SSR-0101599 |
| DSR-0103381 | SSR-0106627 | SSR-0101599 |
| DSR-0103382 | SSR-0104475 | SSR-0106632 |
| DSR-0103383 | SSR-0104475 | SSR-0106633 |
| DSR-0103384 | SSR-0104475 | SSR-0106634 |
| DSR-0103385 | SSR-0104717 | SSR-0106643 |
| DSR-0103386 | SSR-0106626 | SSR-0101599 |
| DSR-0103387 | SSR-0106628 | SSR-0101599 |
| DSR-0103388 | SSR-0104475 | SSR-0106635 |
| DSR-0103389 | SSR-0106525 | SSR-0101599 |
| DSR-0103390 | SSR-0106535 | SSR-0101599 |
| DSR-0103391 | SSR-0106537 | SSR-0101599 |
| DSR-0103392 | SSR-0106538 | SSR-0101599 |
| DSR-0103393 | SSR-0106539 | SSR-0101599 |
| DSR-0103394 | SSR-0104475 | SSR-0106631 |
| DSR-0103395 | SSR-0106521 | SSR-0101599 |
| DSR-0103396 | SSR-0106529 | SSR-0101599 |
| DSR-0103397 | SSR-0106531 | SSR-0101599 |
| DSR-0103399 | SSR-0106180 | SSR-0106595 |
| DSR-0103404 | SSR-0106183 | SSR-0106603 |
| DSR-0103408 | SSR-0106183 | SSR-0106510 |
| DSR-0103409 | SSR-0106183 | SSR-0106604 |
| DSR-0103411 | SSR-0106183 | SSR-0106506 |
| DSR-0103412 | SSR-0106183 | SSR-0106511 |
| DSR-0103414 | SSR-0106183 | SSR-0106508 |
| DSR-0103415 | SSR-0106183 | SSR-0106513 |
| DSR-0103416 | SSR-0106183 | SSR-0106503 |
| DSR-0103418 | SSR-0106183 | SSR-0106598 |
| DSR-0103419 | SSR-0106183 | SSR-0106600 |
| DSR-0103420 | SSR-0106183 | SSR-0106602 |
| DSR-0103424 | SSR-0106183 | SSR-0106509 |
| DSR-0103425 | SSR-0106183 | SSR-0106601 |
| DSR-0103426 | SSR-0106183 | SSR-0106605 |
| DSR-0103427 | SSR-0106183 | SSR-0106606 |
| DSR-0103428 | SSR-0106183 | SSR-0106607 |
| DSR-0103429 | SSR-0106183 | SSR-0106507 |
| DSR-0103430 | SSR-0106183 | SSR-0106597 |
| DSR-0103431 | SSR-0106183 | SSR-0106599 |
| DSR-0103432 | SSR-0106587 | SSR-0106589 |
| DSR-0103712 | SSR-0106224 | SSR-0106206 |
| DSR-0103713 | SSR-0106516 | SSR-0106206 |
| DSR-0103714 | SSR-0106517 | SSR-0106206 |
| DSR-0103715 | SSR-0106514 | SSR-0106206 |
| DSR-0103716 | SSR-0106515 | SSR-0106206 |
| DSR-0103717 | SSR-0106518 | SSR-0106206 |
| DSR-0103718 | SSR-0106512 | SSR-0106206 |
| DSR-0103719 | SSR-0106519 | SSR-0106206 |
| DSR-0103966 | SSR-0107427 | SSR-0107400 |
| DSR-0103967 | SSR-0107427 | SSR-0107419 |
| DSR-0103968 | SSR-0107427 | SSR-0107420 |
| DSR-0103969 | SSR-0107427 | SSR-0107412 |
| DSR-0103970 | SSR-0107427 | SSR-0107413 |
| DSR-0103971 | SSR-0107427 | SSR-0107414 |
| DSR-0103972 | SSR-0107427 | SSR-0107421 |
| DSR-0103973 | SSR-0107427 | SSR-0107415 |
| DSR-0104104 | SSR-0107428 | SSR-0107412 |
| DSR-0104105 | SSR-0108082 | SSR-0108086 |
| DSR-0104106 | SSR-0108084 | SSR-0108086 |
| DSR-0104107 | SSR-0108085 | SSR-0108086 |
| DSR-0104213 | SSR-0106222 | SSR-0106485 |
| DSR-0104214 | SSR-0106222 | SSR-0106564 |
| DSR-0104215 | SSR-0106222 | SSR-0106565 |
| DSR-0104216 | SSR-0106222 | SSR-0106566 |
| DSR-0104217 | SSR-0106222 | SSR-0106568 |
| DSR-0104218 | SSR-0106222 | SSR-0106569 |
| DSR-0104219 | SSR-0106222 | SSR-0106570 |
| DSR-0104220 | SSR-0106222 | SSR-0106571 |
| DSR-0104221 | SSR-0106222 | SSR-0106576 |
| DSR-0104222 | SSR-0106222 | SSR-0106577 |
| DSR-0104223 | SSR-0106222 | SSR-0106578 |
| DSR-0104224 | SSR-0106222 | SSR-0106652 |
| DSR-0104225 | SSR-0106222 | SSR-0106653 |
| DSR-0104226 | SSR-0106222 | SSR-0106579 |
| DSR-0104227 | SSR-0106222 | SSR-0106573 |
| DSR-0104228 | SSR-0106222 | SSR-0106575 |
| DSR-0104229 | SSR-0106222 | SSR-0106679 |
| DSR-0104230 | SSR-0106222 | SSR-0106580 |
| DSR-0104231 | SSR-0106222 | SSR-0106660 |
| DSR-0104232 | SSR-0106222 | SSR-0106503 |
| DSR-0104233 | SSR-0106222 | SSR-0106507 |
| DSR-0104234 | SSR-0106222 | SSR-0106665 |
| DSR-0104235 | SSR-0106222 | SSR-0106666 |
| DSR-0104236 | SSR-0106222 | SSR-0106601 |
| DSR-0104237 | SSR-0106222 | SSR-0106602 |
| DSR-0104238 | SSR-0106222 | SSR-0106661 |
| DSR-0104239 | SSR-0106222 | SSR-0106662 |
| DSR-0104240 | SSR-0106222 | SSR-0106663 |
| DSR-0104241 | SSR-0106222 | SSR-0106664 |

TABLE 1g
Example Oligonucleotides/Compositions

| | | BASE | NAME |
|---|---|---|---|
| ID | HELM | SEQUENCE | |
| ASO-0104323 | RNA1{[moe](T)[sp].[moe]([m5C])[sp].[moe](A)[sp]. | TCACTTTC | WV- |
| | [moe]([m5C])[sp].[moe](T)[sp].[moe](T)[sp].[moe | ATAATGCT | 2782 |
| | ](T)[sp].[moe]([m5C])[sp].[moe](A)[sp].[moe](T) | GG | |
| | [sp].[moe](A)[sp].[moe](A)[sp].[moe](T)[sp]. | | |
| | [moe](G)[sp].[moe]([m5C])[sp].[moe](T)[sp]. | | |
| | [moe](G)[sp].[moe](G)}$$$$V2.0 | | |
| ASO-0109259 | RNA1{m(C)[Ssp].[moe]([m5C])p.[moe](T)p.[moe] | CCTCACTC | WV- |
| | ([m5C])p.m(A)[Ssp].d(C)[Ssp].d(T)[Ssp].d(C)[Rsp]. | ACCCACTC | 8012 |
| | d(A)[Ssp].d(C)[Ssp].d(C)[Rsp].d(C)[Ssp].d(A)[Ssp]. | GCCA | |
| | d(C)[Ssp].d(T)[Ssp].m(C)[Ssp].m(G)[Ssp].m(C)[Ssp]. | | |
| | m(C)[Ssp].m(A)}$$$$V2.0 | | |
| ASO-0100157 | RNA1{m(U)[sp].m(C)[sp].m(A)[sp].m(A)[sp].m(G)[sp]. | UCAAGGAA | WV- |
| or | m(G)[sp].m(A)[sp].m(A)[sp].m(G)[sp].m(A)[sp].m(U) | GAUGGCAU | 942 |
| ASO-0100157- | [sp].m(G)[sp].m(G)[sp].m(C)[sp].m(A)[sp].m(U)[sp]. | UUCU | |
| 004) | m(U)[sp].m(U)[sp].m(C)[sp].m(U)}$$$$V2.0 | | |
| ASO-0136356 | RNA1{d(T)[sp].d(C)[sp].d(G)[sp].d(T)[sp].d(C)[sp]. | TCGTCGTT | WV- |
| | d(G)[sp].d(T)[sp].d(T)[sp].d(T)[sp].d(T)[sp].d(G) | TTGTCGTT | 2021 |
| | [sp].d(T)[sp].d(C)[sp].d(G)[sp].d(T)[sp].d(T)[sp]. | TTGTCGTT | |
| | d(T)[sp].d(T)[sp].d(G)[sp].d(T)[sp].d(C)[sp].d(G) | | |
| | [sp].d(T)[sp].d(T)}$$$$V2.0 | | |

### Notes

Such notation language is described in Zhang, T. et. al. Chem. Inf Model. 2012, 52, 10, 2796-2806 and Milton, J. et al. J. Chem. Inf Model. 2017, 57, 6, 1233-1239. Description, Base Sequence and Stereochemistry/Linkage, due to their length, may be divided into multiple lines in Table 1. Unless otherwise specified, all oligonucleotides in Table 1 are single-stranded. As appreciated by those skilled in the art, nucleoside units are unmodified and contain unmodified nucleobases and 2′-deoxy sugars unless otherwise indicated (e.g., with r, m, etc.); linkages, unless otherwise indicated, are natural phosphate linkages; and acidic/basic groups may independently exist in their salt forms. If a sugar is not specified, the sugar is a natural DNA sugar; and if an internucleotidic linkage is not specified, the internucleotidic linkage is a natural phosphate linkage. Moieties and modifications:
m: 2′-OMe;
f or [fl2r]: 2′-F;
O, PO, p: phosphodiester (phosphate). It can a linkage or be an end group (or a component thereof), e.g., a linkage between a linker and an oligonucleotide chain, an internucleotidic linkage (a natural phosphate linkage), etc. Phosphodiesters are typically indicated with “O” in the Stereochemistry/Linkage column and are typically not marked in the Description column (if it is an end group, e.g., a 5′-end group, it is indicated in the Description and typically not in Stereochemistry/Linkage); if no linkage is indicated in the Description column, it is typically a phosphodiester unless otherwise indicated. Note that a phosphate linkage between a linker (e.g., L001) and an oligonucleotide chain may not be marked in the Description column, but may be indicated with “O” in the Stereochemistry/Linkage column;
*, PS, sp: Phosphorothioate. It can be an end group (if it is an end group, e.g., a 5′-end group, it is indicated in the Description and typically not in Stereochemistry/Linkage), or a linkage, e.g., a linkage between linker (e.g., L001) and an oligonucleotide chain, an internucleotidic linkage (a phosphorothioate internucleotidic linkage), etc.;
R, Rp, or [Rsp]: Phosphorothioate or phosophoryl guanidine in the Rp configuration. Note that *R in Description indicates a single phosphorothioate or phosphoryl guanidine linkage in the Rp configuration;
S, Sp, or [Ssp]: Phosphorothioate or phosphoryl guanidine in the Sp configuration. Note that *S in Description indicates a single phosphorothioate or phosphoryl guanidine linkage in the Sp configuration;
X: stereorandom phosphorothioate or phosphoryl guanidine;
CHEM1: ligand;
CHEM2: 5′-linker;

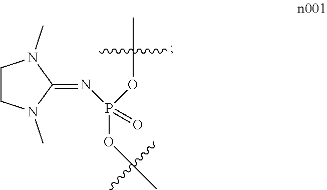 [SMILES: *OP(=O)(N=C1N(C)CCN1C)O*]

nX: stereorandom n001;
nR or n001R or [n001R]: n001 in Rp configuration;
nS or n001S or [n001S]: n001 in Sp configuration;

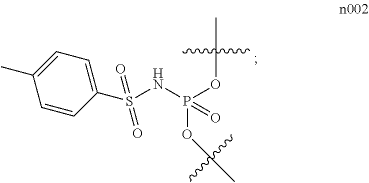 [SMILES: *OP(=O)(NS(=O)(=O)c1ccc(C)cc1)O*]

nX: stereorandom n002;
nR or n002R: n002 in Rp configuration;
nS or n002S: n002 in Sp configuration;

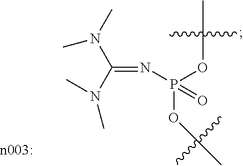 [SMILES: *OP(=O)(N=C(N(C)C)N(C)C)O*]

nX: stereorandom n003;
nR or n003R: n003 in Rp configuration;
nS or n003S: n003 in Sp configuration;

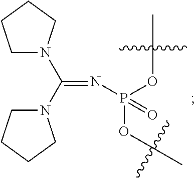 [SMILES: *OP(=O)(N=C(N1CCCC1)N1CCCC1)O*]

nX: stereorandom n004;
nR or n004R: n004 in Rp configuration;
nS or n004S: n004 in Sp configuration;

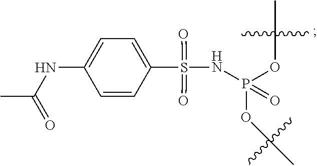 [SMILES: *OP(=O)(NS(=O)(=O)c1ccc(NC(C)=O)cc1)O*]

nX: stereorandom n006;
nR or n006R: n006 in Rp configuration;
nS or n006S: n006 in Sp configuration;

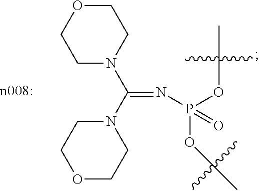 [SMILES: *OP(=O)(N=C(N1CCOCC1)N1CCOCC1)O*]

nX: stereorandom n008;
nR or n008R: n008 in Rp configuration;
nS or n008S: n008 in Sp configuration;

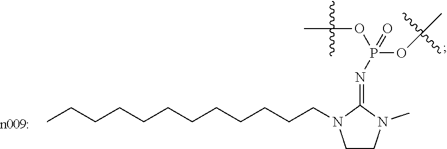 [SMILES: *OP(=O)(/N=C1\N(C)CCN1CCCCCCCCCCCC)O*]

nX: stereorandom n009;
nR or n009R: n009 in Rp configuration;
nS or n009S: n009 in Sp configuration;

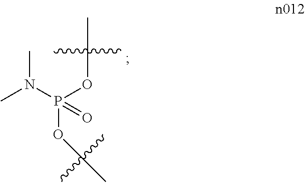 [SMILES: *OP(=O)(O*)N(C)C]

nX: stereorandom n012;
nR or n012R: n012 in Rp configuration;
nS or n012S: n012 in Sp configuration;

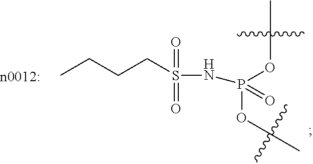 [SMILES: *OP(=O)(NS(=O)(=O)CCCC)O*]

nX: stereorandom n020;
nR or n020R: n020 in Rp configuration;
nS or n020S: n020 in Sp configuration;

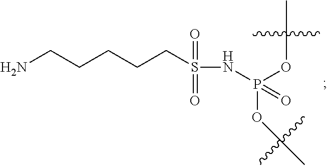 [SMILES: *OP(=O)(NS(=O)(=O)CCCCCN)O*]

nX: stereorandom n021;
nR or n021R: n021 in Rp configuration;
nS or n021S: n021 in Sp configuration;

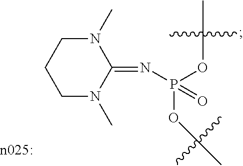 [SMILES: *OP(=O)(N=C1N(C)CCCN1C)O*]

nX: stereorandom n025;
nR or n025R: n025 in Rp configuration;
nS or n025S: n025 in Sp configuration;

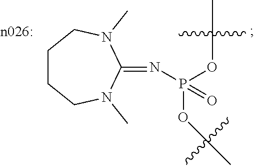 [SMILES: *OP(=O)(N=C1N(C)CCCCN1C)O*]

nX: stereorandom n026;
nR or n026R: n026 in Rp configuration;
nS or n026S: n026 in Sp configuration;

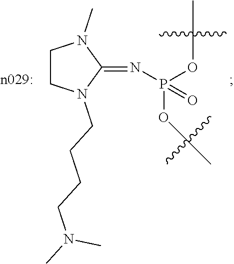 [SMILES: *OP(=O)(/N=C1/N(C)CCN1CCCCN(C)C)O*]

nX: stereorandom n029;
nR or n029R: n029 in Rp configuration;
nS or n029S: n029 in Sp configuration;

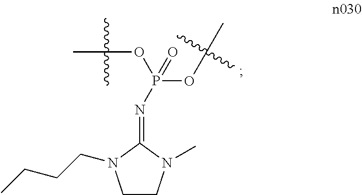 [SMILES: *OP(=O)(/N=C1\N(C)CCN1CCCC)O*]

nX: stereorandom n030;
nR or n030R: n030 in Rp configuration;
nS or n030S: n030 in Sp configuration;

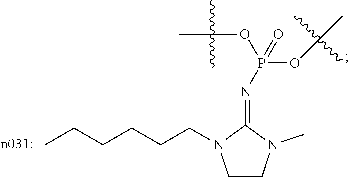 [SMILES: *OP(=O)(/N=C1\N(C)CCN1CCCCCC)O*]

nX: stereorandom n031;
nR or n031R: n031 in Rp configuration;
nS or n031S: n031 in Sp configuration;

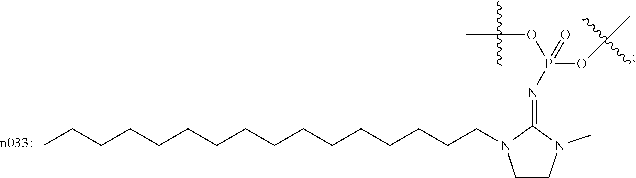 [SMILES: *OP(=O)(/N=C1\N(C)CCN1CCCCCCCCCCCCCCCC)O*]

nX: stereorandom n033;
nR or n033R: n033 in Rp configuration;
nS or n033S: n033 in Sp configuration;

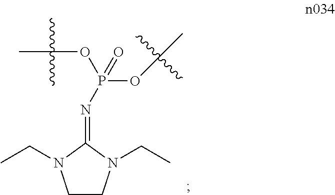 [SMILES: *OP(=O)(N=C1N(CC)CCN1CC)O*]

nX: stereorandom n034;
nR or n034R: n034 in Rp configuration;
nS or n034S: n034 in Sp configuration;

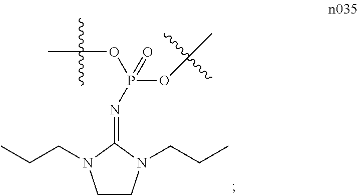 [SMILES: *OP(=O)(N=C1N(CCC)CCN1CCC)O*]

nX: stereorandom n035;
nR or n035R: n035 in Rp configuration;
nS or n035S: n035 in Sp configuration;

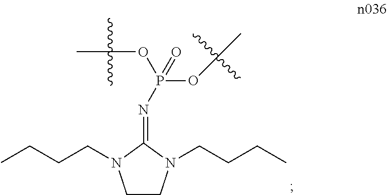 [SMILES: *OP(=O)(N=C1N(CCCC)CCN1CCCC)O*]

nX: stereorandom n036;
nR or n036R: n036 in Rp configuration;
nS or n036S: n036 in Sp configuration;

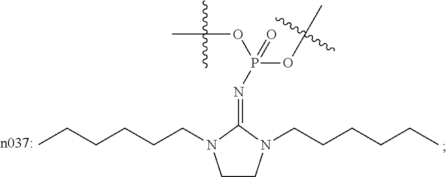 [SMILES: *OP(=O)(N=C1N(CCCCCC)CCN1CCCCCC)O*]

nX: stereorandom n037;
nR or n037R: n037 in Rp configuration;
nS or n037S: n037 in Sp configuration;

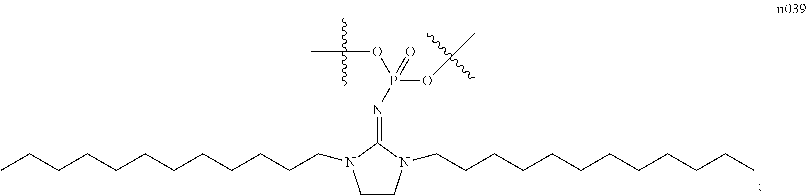 [SMILES: *OP(=O)(N=C1N(CCCCCCCCCCCC)CCN1CCCCCCCCCCCC)O*]

nX: stereorandom n039;
nR or n039R: n039 in Rp configuration;
nS or n039S: n039 in Sp configuration;

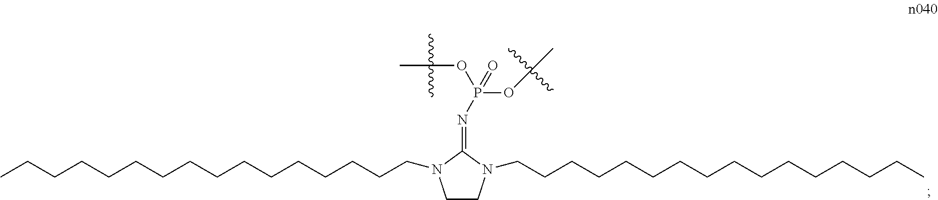 [SMILES: *OP(=O)(N=C1N(CCCCCCCCCCCCCCCC)CCN1CCCCCCCCCCCCCCCC)O*]

nX: stereorandom n040;
nR or n040R: n040 in Rp configuration;
nS or n040S: n040 in Sp configuration;

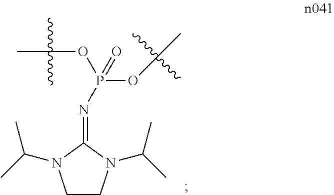 [SMILES: *OP(=O)(N=C1N(C(C)C)CCN1C(C)C)O*]

nX: stereorandom n041;
nR or n041R: n041 in Rp configuration;
nS or n041S: n041 in Sp configuration;

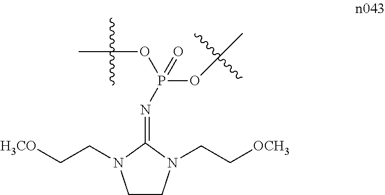 [SMILES: *OP(=O)(N=C1N(CCOC)CCN1CCOC)O*]

nX: stereorandom n043;
nR or n043R: n043 in Rp configuration;
nS or n043S: n043 in Sp configuration;

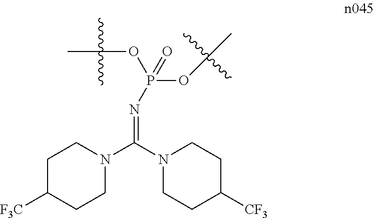 [SMILES: *OP(=O)(N=C(N1CCC(C(F)(F)F)CC1)N1CCC(C(F)(F)F)CC1)O*]

nX: stereorandom n045;
nR or n045R: n045 in Rp configuration;
nS or n045S: n045 in Sp configuration;

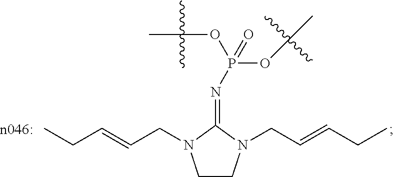 [SMILES: *OP(=O)(N=C1N(C/C=C/CC)CCN1C/C=C/CC)O*]

nX: stereorandom n046;
nR or n046R: n046 in Rp configuration;
nS or n046S: n046 in Sp configuration;

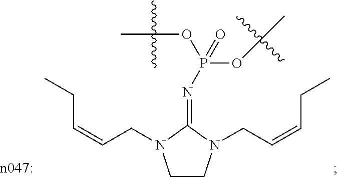 [SMILES: *OP(=O)(N=C1N(C/C=C\CC)CCN1C/C=C\CC)O*]

nX: stereorandom n047;
nR or n047R: n047 in Rp configuration;
nS or n047S: n047 in Sp configuration;

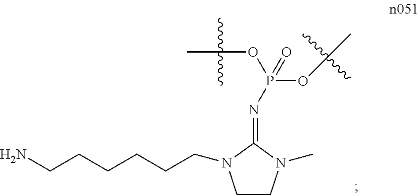 [SMILES: *OP(=O)(/N=C1\N(C)CCN1CCCCCCN)O*]

nX: stereorandom n051;
nR or n051R: n051 in Rp configuration;
nS or n051S: n051 in Sp configuration;

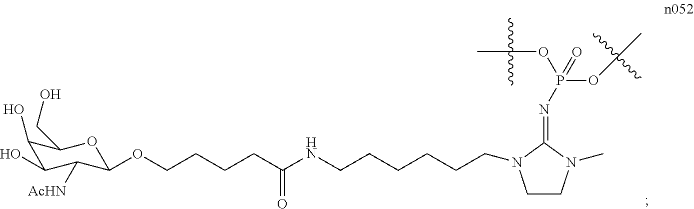 [SMILES: *OP(=O)(/N=C1\N(C)CCN1CCCCCCNC(=O)CCCCOC1OC(CO)C(O)C(O)C1NC(C)=O)O*]

nX: stereorandom n052;
nR or n052R: n052 in Rp configuration;
nS or n052S: n052 in Sp configuration;

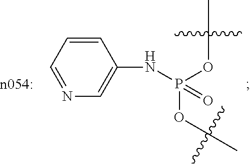 [SMILES: *OP(=O)(Nc1cccnc1)O*]

nX: stereorandom n054;
nR or n054R: n054 in Rp configuration;
nS or n054S: n054 in Sp configuration;

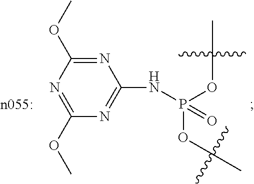 [SMILES: *OP(=O)(Nc1nc(OC)nc(OC)n1)O*]

nX: stereorandom n055;
nR or n055R: n055 in Rp configuration;
nS or n055S: n055 in Sp configuration;

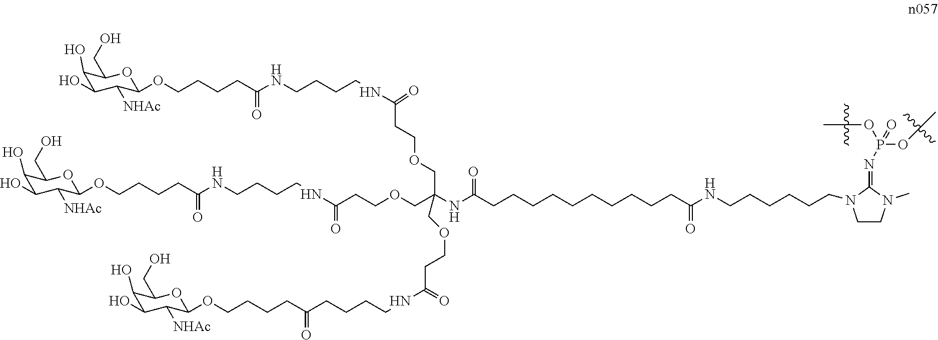 [SMILES: *OP(=O)(/N=C1\N(C)CCN1CCCCCCNC(=O)CCCCCCCCCCC(=O)NC(COCCC(=O)NCCCCNC(=O)CCCCOC1OC(CO)C(O)C(O)C1NC(C)=O)(COCCC(=O)NCCCCNC(=O)CCCCOC1OC(CO)C(O)C(O)C1NC(C)=O)COCCC(=O)NCCCCC(=O)CCCCOC1OC(CO)C(O)C(O)C1NC(C)=O)O*]

nX: stereorandom n058;
nR or n058R: n058 in Rp configuration;
nS or n058S: n058 in Sp configuration;

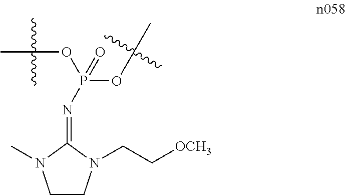 [SMILES: *OP(=O)(/N=C1/N(C)CCN1CCOC)O*]

nX: stereorandom n060;
nR or n060R: n060 in Rp configuration;
nS or n060S: n060 in Sp configuration;

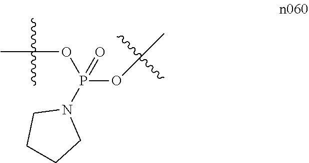 [SMILES: *OP(=O)(O*)N1CCCC1]

nX: stereorandom n061;
nR or n061R: n061 in Rp configuration;
nS or n061S: n061 in Sp configuration;

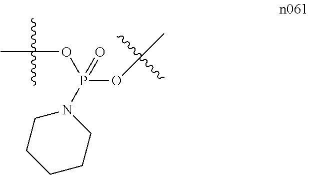 [SMILES: *OP(=O)(O*)N1CCCCC1]

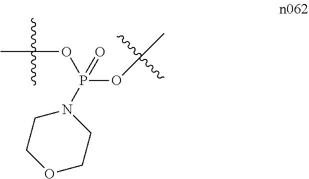 [SMILES: *OP(=O)(O*)N1CCOCC1]

nX: stereorandom n062;
nR or n062R: n062 in Rp configuration;
nS or n062S: n062 in Sp configuration;

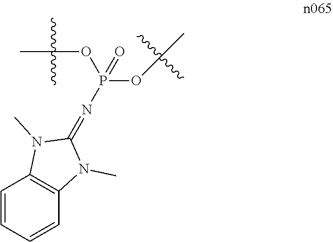 [SMILES: *OP(=O)(N=c1n(C)c2ccccc2n1C)O*]

nX: stereorandom n065;
nR or n065R: n065 in Rp configuration;
nS or n065S: n065 in Sp configuration;

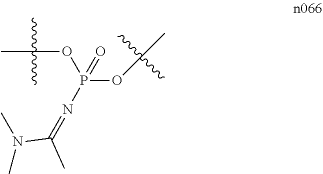 [SMILES: *OP(=O)(N=C(C)N(C)C)O*]

nX: stereorandom n066;
nR or n066R: n066 in Rp configuration;
nS or n066S: n066 in Sp configuration;

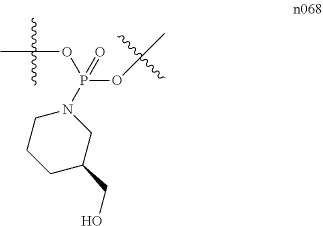 [SMILES: *OP(=O)(O*)N1CCC[C@H](CO)C1]

nX: stereorandom n068;
nR or n068R: n068 in Rp configuration;
nS or n068S: n068 in Sp configuration;

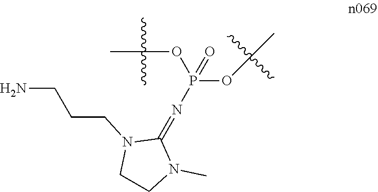 [SMILES: *OP(=O)(N=C1N(C)CCN1CCCN)O*]

nX: stereorandom n069;
nR or n069R: n069 in Rp configuration;
nS or n069S: n069 in Sp configuration;

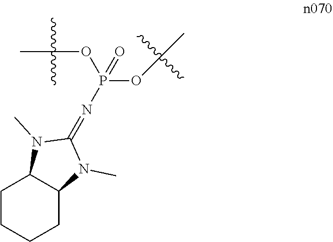 [SMILES: *OP(=O)(N=C1N(C)[C@@H]2CCCC[C@@H]2N1C)O*]

nX: stereorandom n070;
nR or n070R: n070 in Rp configuration;
nS or n070S: n070 in Sp configuration;

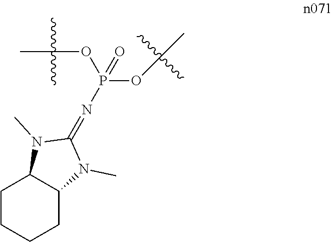 [SMILES: *OP(=O)(N=C1N(C)[C@@H]2CCCC[C@H]2N1C)O*]

nX: stereorandom n071;
nR or n071R: n071 in Rp configuration;
nS or n071S: n071 in Sp configuration;

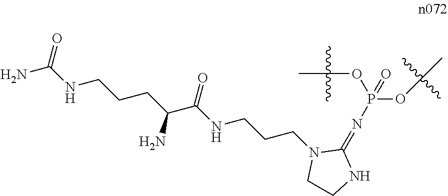 [SMILES: *OP(=O)(N=C1NCCN1CCCNC(=O)[C@@H](N)CCCNC(N)=O)O*]

nX: stereorandom n072;
nR or n072R: n072 in Rp configuration;
nS or n072S: n072 in Sp configuration;

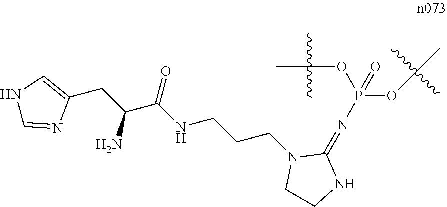 [SMILES: *OP(=O)(N=C1NCCN1CCCNC(=O)[C@@H](N)Cc1c[nH]cn1)O*]

nX: stereorandom n073;
nR or n073R: n073 in Rp configuration;
nS or n073S: n073 in Sp configuration;

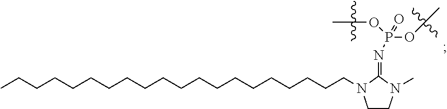 [SMILES: *OP(=O)(/N=C1\N(C)CCN1CCCCCCCCCCCCCCCCCCCC)O*]

nX: stereorandom n076;
nR or n076R: n076 in Rp configuration;
nS or n076S: n076 in Sp configuration;

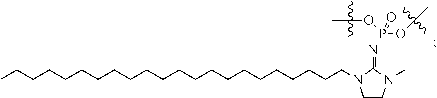 [SMILES: *OP(=O)(/N=C1\N(C)CCN1CCCCCCCCCCCCCCCCCCCCCC)O*]

nX: stereorandom n077;
nR or n077R: n077 in Rp configuration;
nS or n077S: n077 in Sp configuration;
X: stereorandom phosphorothioate or phosphoryl guanidine;

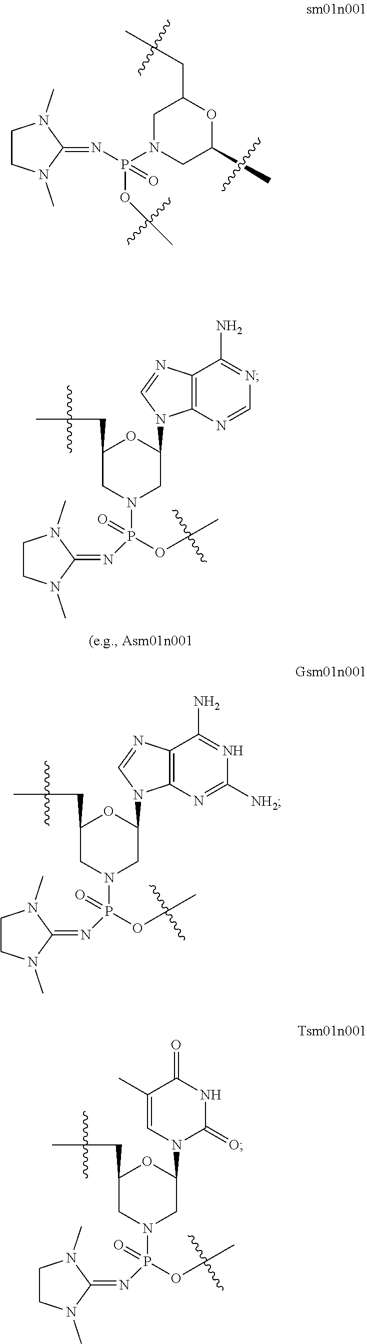

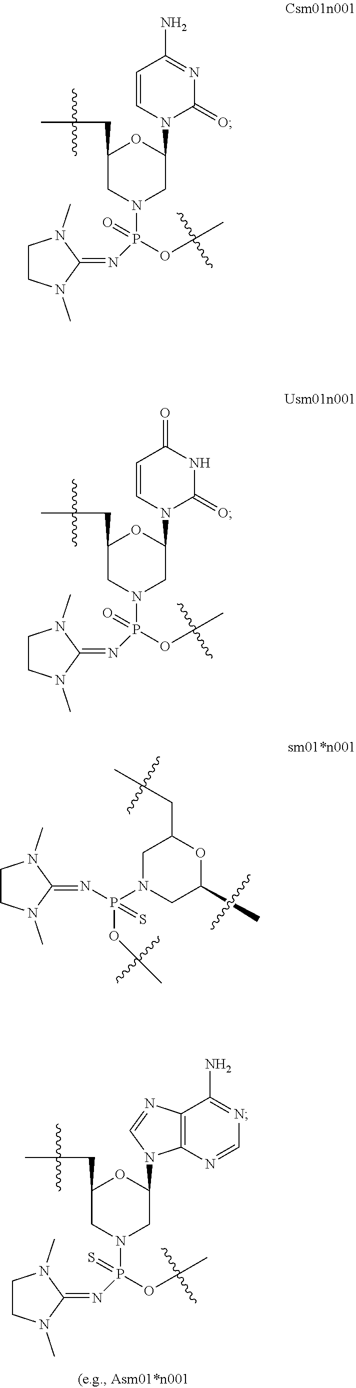

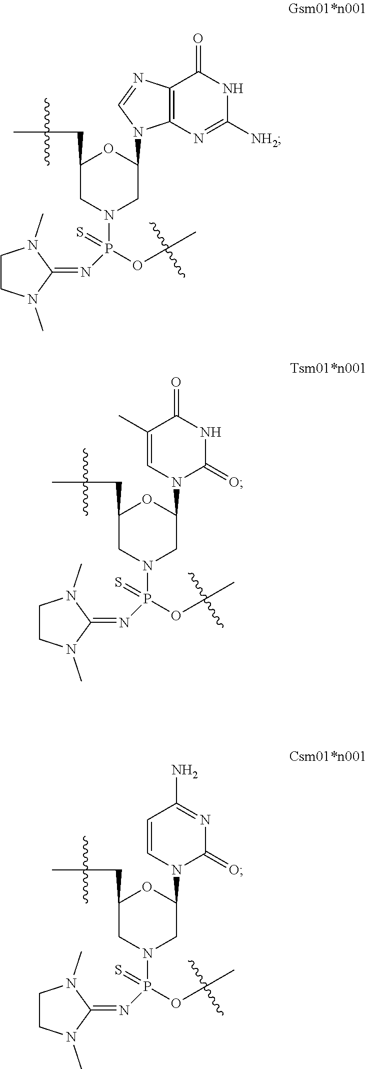 [SMILES: *C[C@@H]1CN(P(=S)(N=C2N(C)CCN2C)O*)C[C@H](n2cnc3c(=O)[nH]c(N)nc32)O1 ; *C[C@@H]1CN(P(=S)(N=C2N(C)CCN2C)O*)C[C@H](n2cc(C)c(=O)[nH]c2=O)O1 ; *C[C@@H]1CN(P(=S)(N=C2N(C)CCN2C)O*)C[C@H](n2ccc(N)nc2=O)O1]

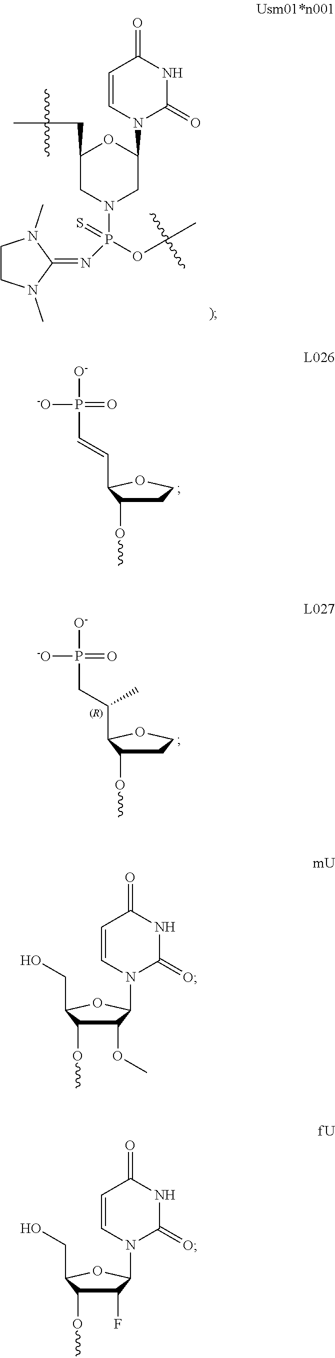

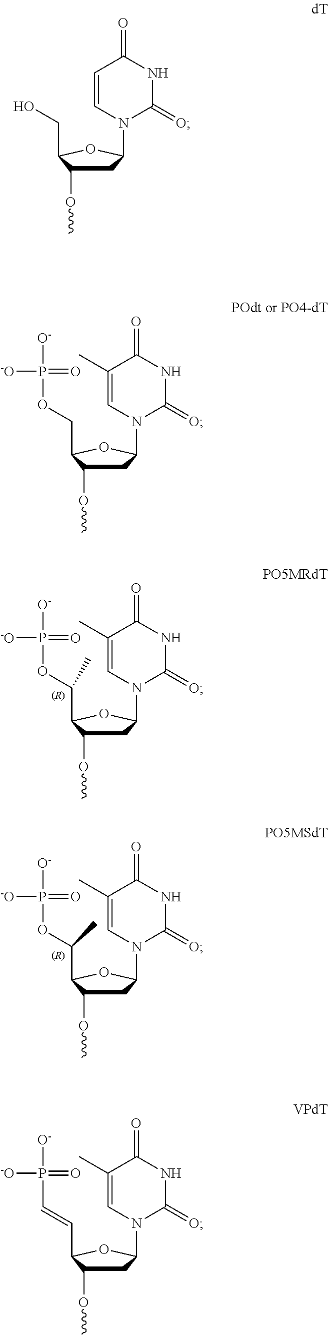

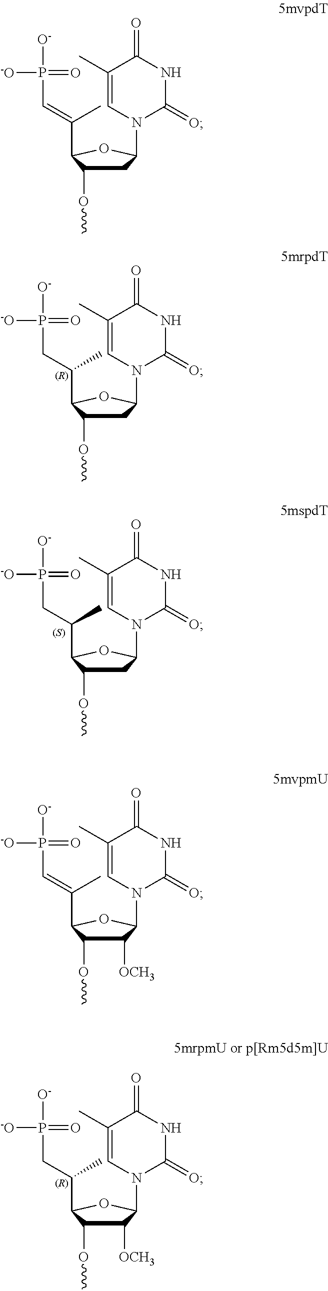

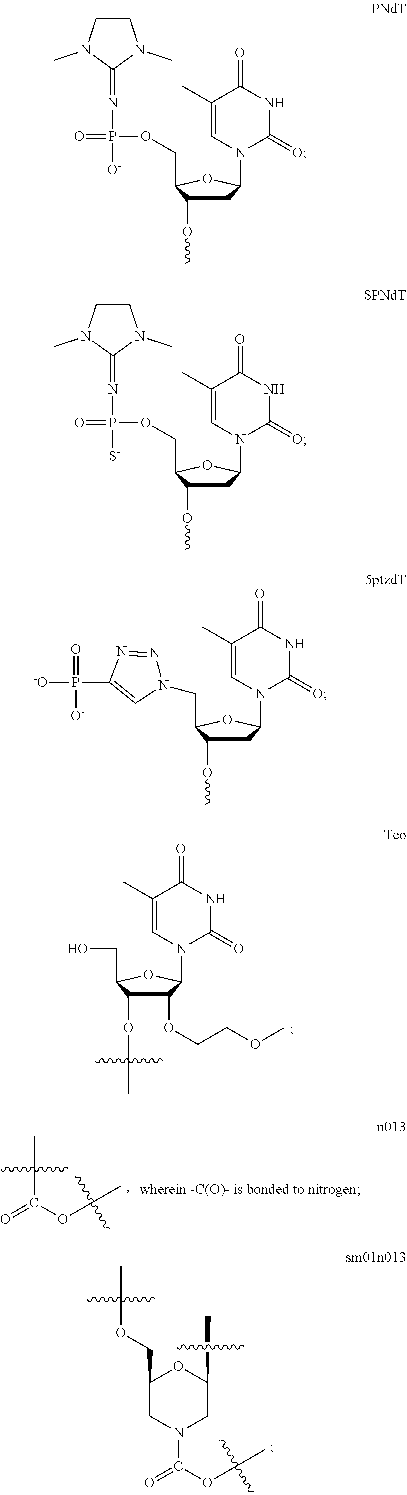

i.e., morpholine carbamate internucleotidic linkage

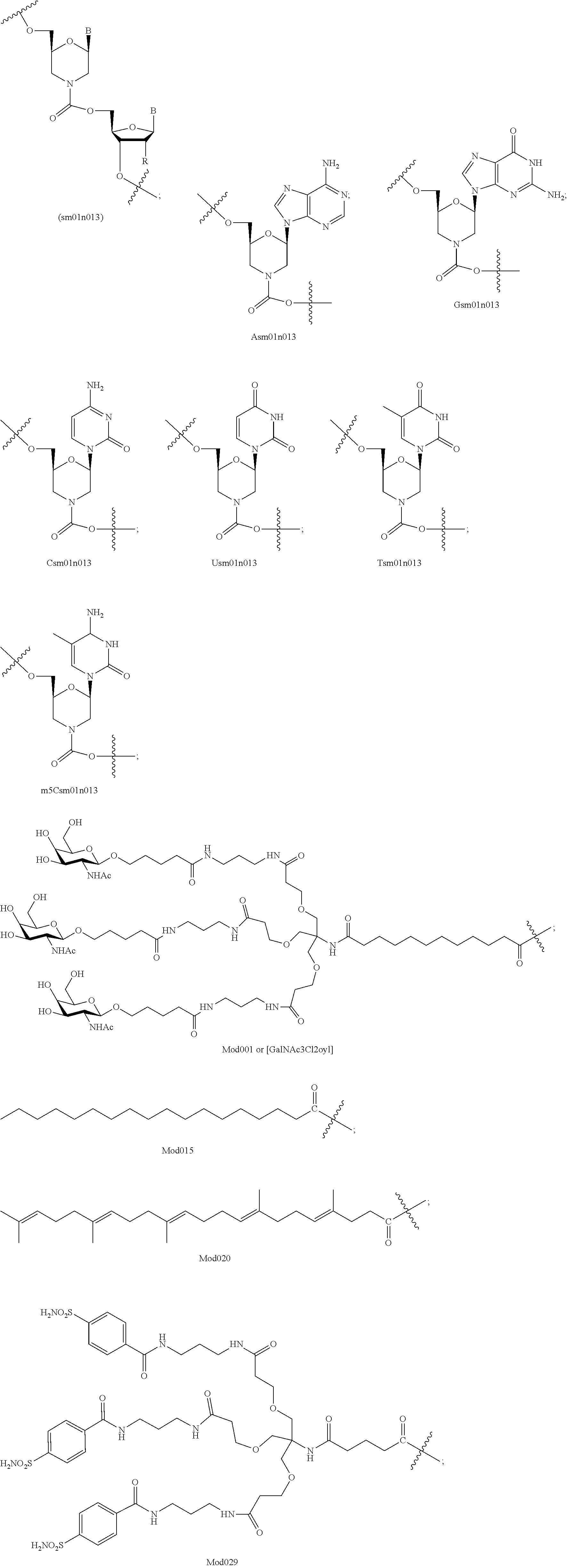

L001 or nC6o: —NH—(CH₂)₆-linker (C6 linker, C6 amine linker or C6 amino linker), connected to Mod (e.g., Mod001) through —NH—, and, in the case of, for example, VW-38061, the 5′-end of the oligonucleotide chain through a phosphate linkage (O or PO). For example, in WV-38061, L001 is connected to Mod001 through —NH— (forming an amide group —C(O)—NH—), and is connected to the oligonucleotide chain through a phosphate linkage (O).

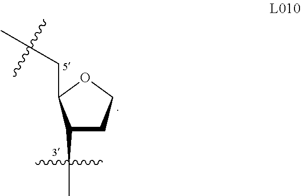

In some embodiments, when L010 is present in the middle of an oligonucleotide, it is bonded to internucleotidic linkages as other sugars (e.g., DNA sugars), e.g., its 5′-carbon is connected to another unit (e.g., 3′ of a sugar) and its 3′-carbon is connected to another unit (e.g., a 5′-carbon of a carbon) independently, e.g., via a linkage (e.g., a phosphate linkage (O or PO) or a phosphorothioate linkage (can be either not chirally controlled or chirally controlled (Sp or Rp)));
L012:—CH₂CH₂OCH₂CH₂OCH₂CH₂—. When L012 is present in the middle of an oligonucleotide, each of its two ends is independently bonded to an internucleotidic linkage (e.g., a phosphate linkage (O or PO) or a phosphorothioate linkage (can be either not chirally controlled or chirally controlled (Sp or Rp)));

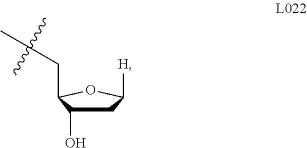

wherein L022 is connected to the rest of a molecule through a phosphate unless indicated otherwise;
L023: HO—(CH₂)₆—, wherein CH₂ is connected to the rest of a molecule through a phosphate unless indicated otherwise. For example, in WV-42644 (wherein the O in OnRnRnRnRSSSSSSSSSSSSSSSSSSnRSSSSSnRSSnR indicates a phosphate linkage connecting L023 to the rest of the molecule);

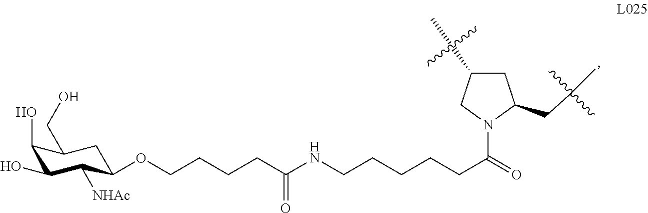

wherein the —CH₂— connection site is utilized as a C5 connection site of a sugar (e.g., a DNA sugar) and is connected to another unit (e.g., 3′ of a sugar), and the connection site on the ring is utilized as a C3 connection site and is connected to another unit (e.g., a 5′-carbon of a carbon), each of which is independently, e.g., via a linkage (e.g., a phosphate linkage (O or PO) or a phosphorothioate linkage (can be either not chirally controlled or chirally controlled (Sp or Rp))). When L025 is at a 5′-end without any modifications, its —CH₂-connection site is bonded to —OH. For example, L025L025L025—in various oligonucleotides has the structure of

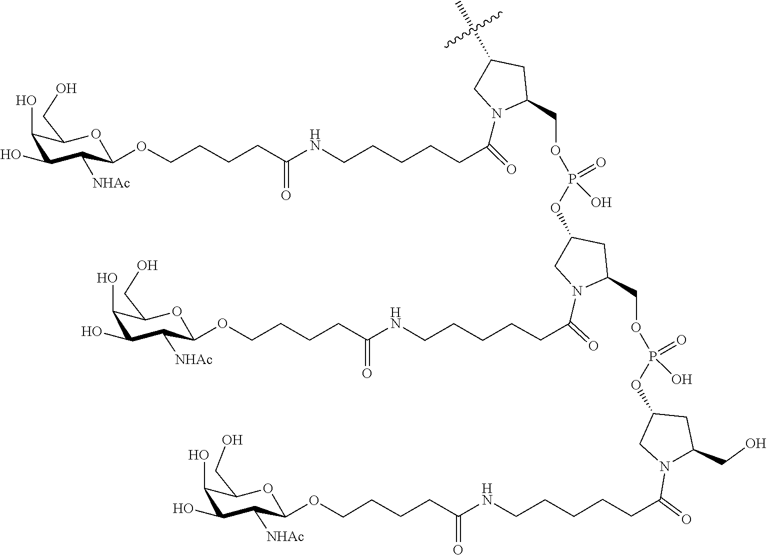

(may exist as various salt forms) and is connected to 5′-carbon of an oligonucleotide chain via a linkage as indicated (e.g., a phosphate linkage (O or PO) or a phosphorothioate linkage (can be either not chirally controlled or chirally controlled (Sp or Rp)));

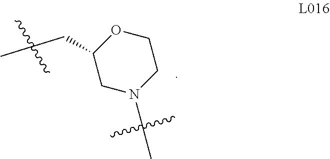

wherein L016 is connected to the rest of a molecule through a phosphate unless indicated otherwise; L016 is utilized with n001 to form L016n001, which has the structure of

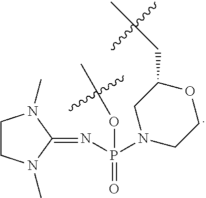 [SMILES: *C[C@H]1CN(P(=O)(N=C2N(C)CCN2C)O*)CCO1]

### 1.1.1 Double Stranded Oligonucleotide Lengths

As appreciated by those skilled in the art, ds oligonucleotides can be of various lengths to provide desired properties and/or activities for various uses. Many technologies for assessing, selecting and/or optimizing ds oligonucleotide length are available in the art and can be utilized in accordance with the present disclosure. As demonstrated herein, in certain embodiments, dsRNAi oligonucleotides are of suitable lengths to hybridize with their targets and reduce levels of their targets and/or an encoded product thereof. In certain embodiments, a ds oligonucleotide is long enough to recognize a target nucleic acid (e.g., a target mRNA). In certain embodiments, a ds oligonucleotide is sufficiently long to distinguish between a target nucleic acid and other nucleic acids (e.g., a nucleic acid having a base sequence which is not a target sequence) to reduce off-target effects. In certain embodiments, a dsRNAi oligonucleotide is sufficiently short to reduce complexity of manufacture or production and to reduce cost of products.

In certain embodiments, the base sequence of a ds oligonucleotide is about 10-500 nucleobases in length. In certain embodiments, a base sequence is about 10-500 nucleobases in length. In certain embodiments, a base sequence is about 10-50 nucleobases in length. In certain embodiments, a base sequence is about 15-50 nucleobases in length. In certain embodiments, a base sequence is from about 15 to about 30 nucleobases in length. In certain embodiments, abase sequence is from about 10 to about 25 nucleobases in length. In certain embodiments, a base sequence is from about 15 to about 22 nucleobases in length. In certain embodiments, a base sequence is about 10, 11, 12, 13, 14, 15, 16, 17, 18, 19, 20, 21, 22, 23, 24, or 25 nucleobases in length. In certain embodiments, a base sequence is about 18 nucleobases in length. In certain embodiments, a base sequence is about 19 nucleobases in length. In certain embodiments, a base sequence is about 20 nucleobases in length. In certain embodiments, a base sequence is about 21 nucleobases in length. In certain embodiments, a base sequence is about 22 nucleobases in length. In certain embodiments, a base sequence is about 23 nucleobases in length. In certain embodiments, a base sequence is about 24 nucleobases in length. In certain embodiments, a base sequence is about 25 nucleobases in length. In certain embodiments, each nucleobase is optionally substituted A, T, C, G, U, or an optionally substituted tautomer of A, T, C, G, or U.

### 2.2.3. Internucleotidic Linkages

In certain embodiments, ds oligonucleotides comprise base modifications, sugar modifications, and/or internucleotidic linkage modifications. Various internucleotidic linkages can be utilized in accordance with the present disclosure to link units comprising nucleobases, e.g., nucleosides. In certain embodiments, provided ds oligonucleotides comprise both one or more modified internucleotidic linkages and one or more natural phosphate linkages. As widely known by those skilled in the art, natural phosphate linkages are widely found in natural DNA and RNA molecules; they have the structure of —OP(O)(OH)O—, connect sugars in the nucleosides in DNA and RNA, and may be in various salt forms, for example, at physiological pH (about 7.4), natural phosphate linkages are predominantly exist in salt forms with the anion being —OP(O)(O⁻)O—. A modified internucleotidic linkage, or a non-natural phosphate linkage, is an internucleotidic linkage that is not natural phosphate linkage or a salt form thereof. Modified internucleotidic linkages, depending on their structures, may also be in their salt forms. For example, as appreciated by those skilled in the art, phosphorothioate internucleotidic linkages which have the structure of —OP(O)(SH)O— may be in various salt forms, e.g., at physiological pH (about 7.4) with the anion being —OP(O)(S⁻)O—.

In certain embodiments, a ds oligonucleotide comprises an internucleotidic linkage which is a modified internucleotidic linkage, e.g., phosphorothioate, phosphorodithioate, methylphosphonate, phosphoroamidate, thiophosphate, 3′-thiophosphate, or 5′-thiophosphate.

In certain embodiments, a modified internucleotidic linkage is a chiral internucleotidic linkage which comprises a chiral linkage phosphorus. In certain embodiments, a chiral internucleotidic linkage is a phosphorothioate linkage. In certain embodiments, a chiral internucleotidic linkage is a non-negatively charged internucleotidic linkage. In certain embodiments, a chiral internucleotidic linkage is a neutral internucleotidic linkage. In certain embodiments, a chiral internucleotidic linkage is chirally controlled with respect to its chiral linkage phosphorus. In certain embodiments, a chiral internucleotidic linkage is stereochemically pure with respect to its chiral linkage phosphorus. In certain embodiments, a chiral internucleotidic linkage is not chirally controlled. In certain embodiments, a pattern of backbone chiral centers comprises or consists of positions and linkage phosphorus configurations of chirally controlled internucleotidic linkages (Rp or Sp) and positions of achiral internucleotidic linkages (e.g., natural phosphate linkages).

In certain embodiments, an internucleotidic linkage comprises a P-modification, wherein a P-modification is a modification at a linkage phosphorus. In certain embodiments, a modified internucleotidic linkage is a moiety which does not comprise a phosphorus but serves to link two sugars or two moieties that each independently comprises a nucleobase, e.g., as in peptide nucleic acid (PNA).

In certain embodiments, a ds oligonucleotide comprises a modified internucleotidic linkage, e.g., those having the structure of Formula I, I-a, I-b, or I-c and described herein and/or in: WO 2018/022473, WO 2018/098264, WO 2018/223056, WO 2018/223073, WO 2018/223081, WO 2018/237194, WO 2019/032607, WO 2019/055951, WO 2019/075357, WO 2019/200185, WO 2019/217784, and/or WO 2019/032612, the internucleotidic linkages (e.g., those of Formula I, I-a, I-b, I-c, etc.) of each of which are independently incorporated herein by reference. In certain embodiments, a modified internucleotidic linkage is a chiral internucleotidic linkage. In certain embodiments, a modified internucleotidic linkage is a phosphorothioate internucleotidic linkage.

In certain embodiments, a modified internucleotidic linkage is a non-negatively charged internucleotidic linkage. In certain embodiments, provided ds oligonucleotides comprise one or more non-negatively charged internucleotidic linkages. In certain embodiments, a non-negatively charged internucleotidic linkage is a positively charged internucleotidic linkage. In certain embodiments, a non-negatively charged internucleotidic linkage is a neutral internucleotidic linkage. In certain embodiments, the present disclosure provides ds oligonucleotides comprising one or more neutral internucleotidic linkages. In certain embodiments, a non-negatively charged internucleotidic linkage has the structure of Formula I-n-1, I-n-2, I-n-3, I-n-4, II, II-a-1, II-a-2, II-b-1, II-b-2, II-c-1, II-c-2, II-d-1, II-d-2, etc., or a salt form thereof, as described herein and/or in U.S. Pat. Nos. 9,394,333, 9,744,183, 9,605,019, 9,982,257, US 20170037399, US 20180216108, US 20180216107, U.S. Pat. No. 9,598,458, WO 2017/062862, WO 2018/067973, WO 2017/160741, WO 2017/192679, WO 2017/210647, WO 2018/098264, WO 2018/022473, WO 2018/223056, WO 2018/223073, WO 2018/223081, WO 2018/237194, WO 2019/032607, WO2019/032612, WO 2019/055951, WO 2019/075357, WO 2019/200185, WO 2019/217784, and/or WO 2019/032612, the non-negatively charged internucleotidic linkages (e.g., those of Formula I-n-1, I-n-2, I-n-3, I-n-4, II, II-a-1, II-a-2, II-b-1, II-b-2, II-c-1, II-c-2, II-d-1, II-d-2, etc., or a suitable salt form thereof) of each of which are independently incorporated herein by reference.

In certain embodiments, a non-negatively charged internucleotidic linkage can improve the delivery and/or activities (e.g., adenosine editing activity).

In certain embodiments, a modified internucleotidic linkage (e.g., a non-negatively charged internucleotidic linkage) comprises optionally substituted triazolyl. In certain embodiments, a modified internucleotidic linkage (e.g., a non-negatively charged internucleotidic linkage) comprises optionally substituted alkynyl. In certain embodiments, a modified internucleotidic linkage comprises a triazole or alkyne moiety. In certain embodiments, a triazole moiety, e.g., a triazolyl group, is optionally substituted. In certain embodiments, a triazole moiety, e.g., a triazolyl group) is substituted. In certain embodiments, a triazole moiety is unsubstituted. In certain embodiments, a modified internucleotidic linkage comprises an optionally substituted cyclic guanidine moiety. In certain embodiments, a modified internucleotidic linkage has the structure of

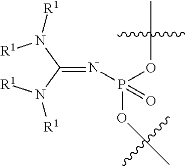

and is optionally chirally controlled, wherein R¹ is -L-R′, wherein L is L^{B} as described herein, and R′ is as described herein. In certain embodiments, each R¹ is independently R′. In certain embodiments, each R′ is independently R. In certain embodiments, two R¹ are R and are taken together to form a ring as described herein. In certain embodiments, two R¹ on two different nitrogen atoms are R and are taken together to form a ring as described herein. In certain embodiments, R¹ is independently optionally substituted C₁₋₆ aliphatic as described herein. In certain embodiments, R¹ is methyl. In certain embodiments, two R′ on the same nitrogen atom are R and are taken together to form a ring as described herein. In certain embodiments, a modified internucleotidic linkage has the structure of

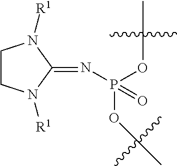

and is optionally chirally controlled. In certain embodiments,

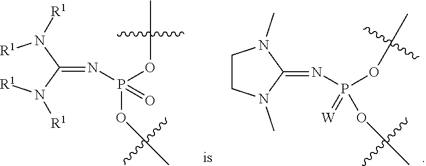

In certain embodiments, a modified internucleotidic linkage comprises an optionally substituted cyclic guanidine moiety and has the structure of:

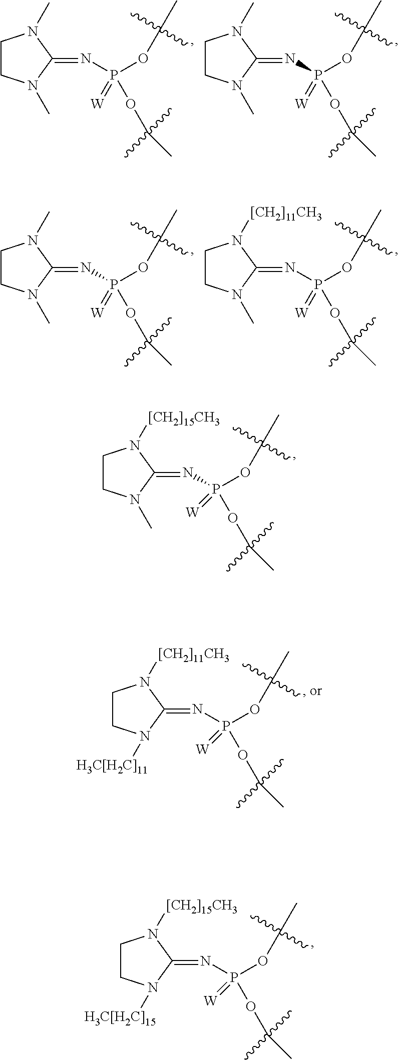

wherein W is O or S. In certain embodiments, W is O. In certain embodiments, W is S. In certain embodiments, a non-negatively charged internucleotidic linkage is stereochemically controlled.
In certain embodiments, a modified internucleotidic linkage has the structure of

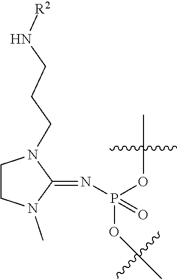

and is optionally chirally controlled

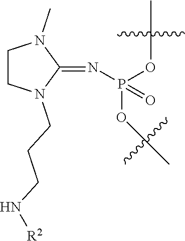

and is optionally chirally controlled,

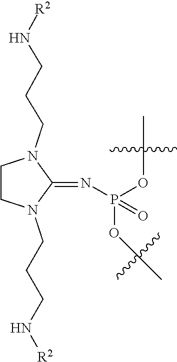

and is optionally chirally controlled,

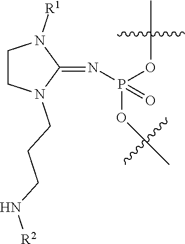

and is optionally chirally controlled, or

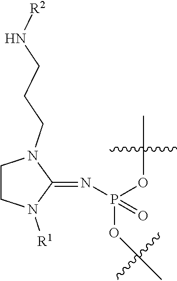

and is optionally chirally controlled wherein R² is H, C6-amine,

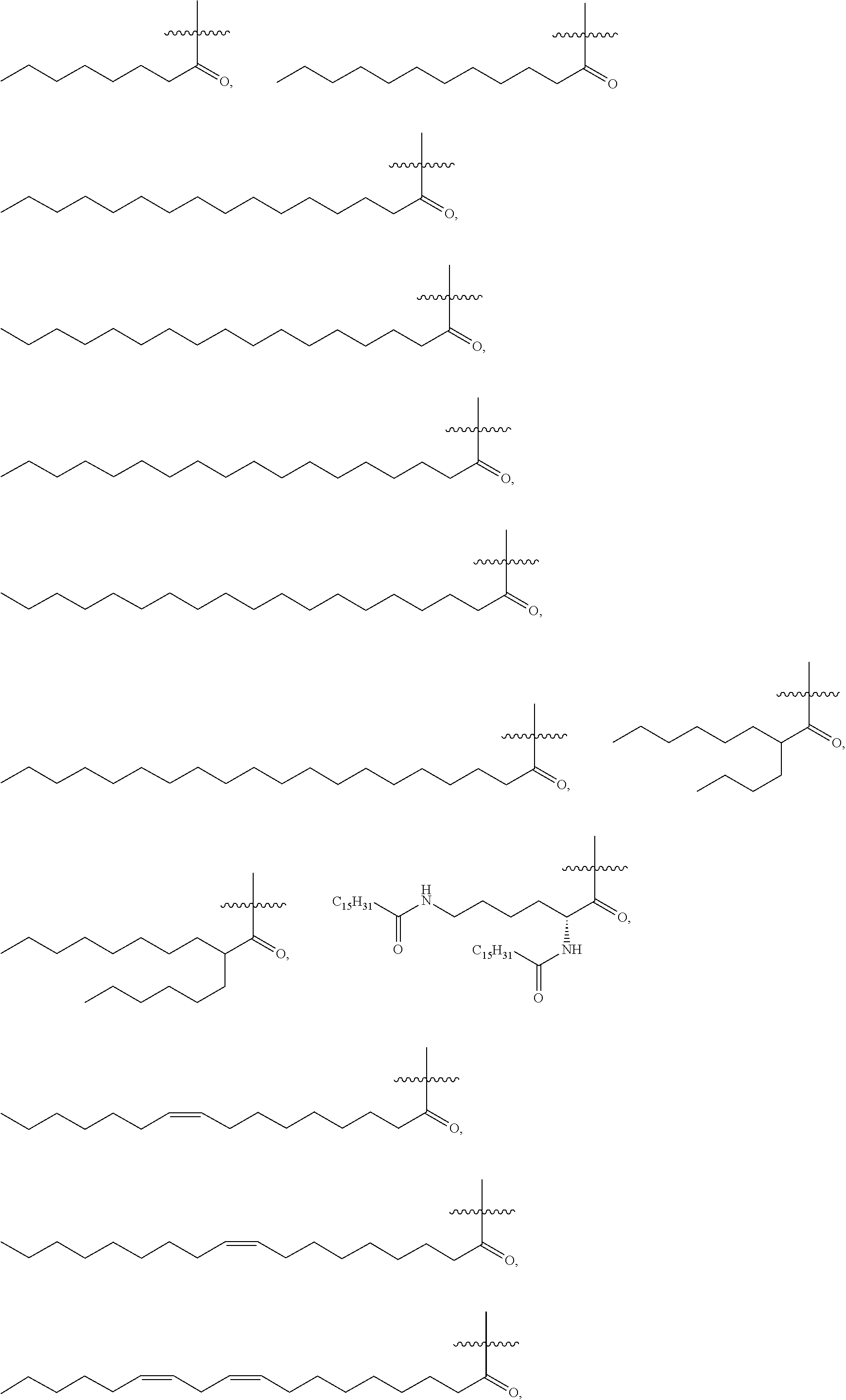

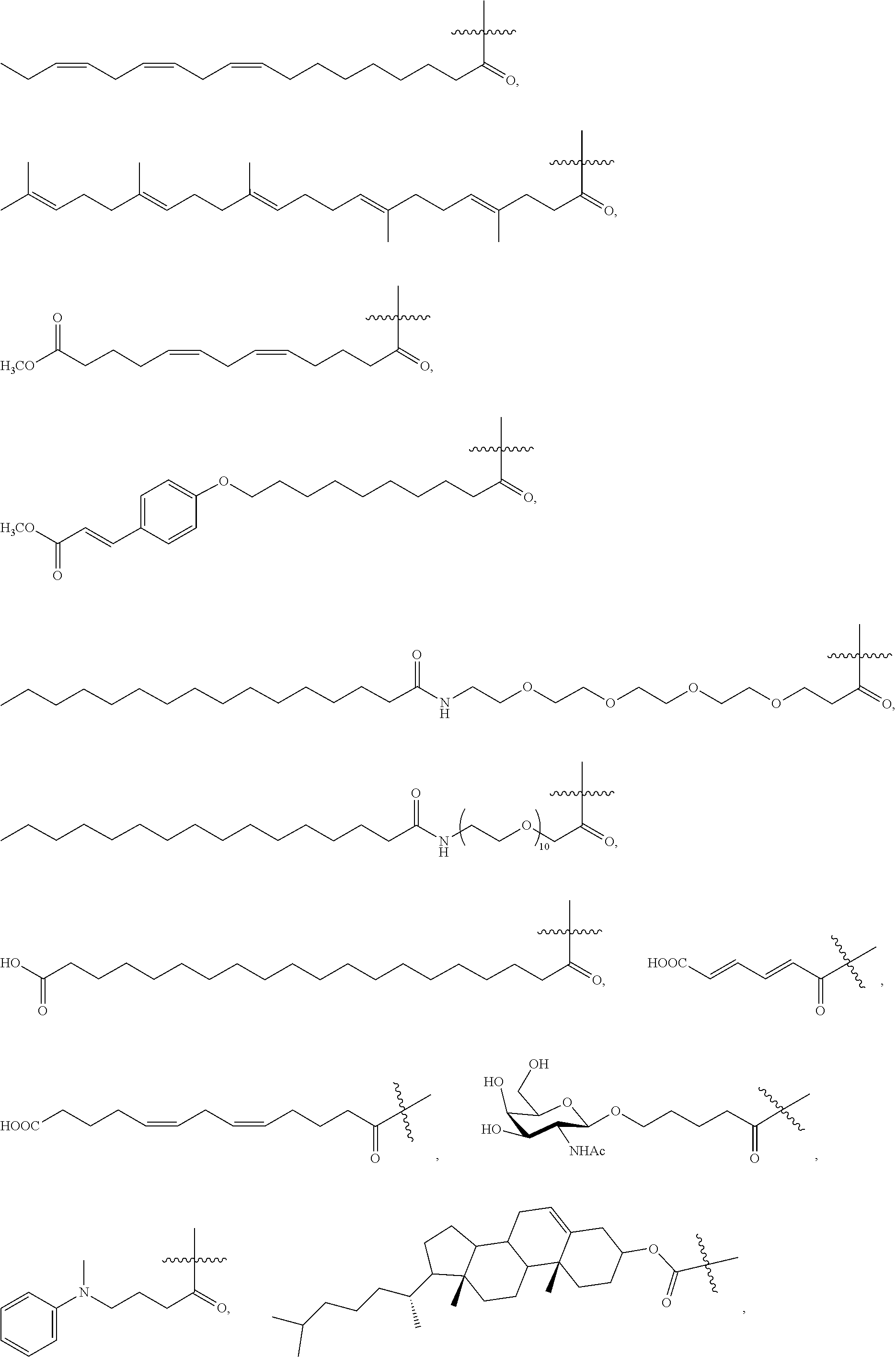

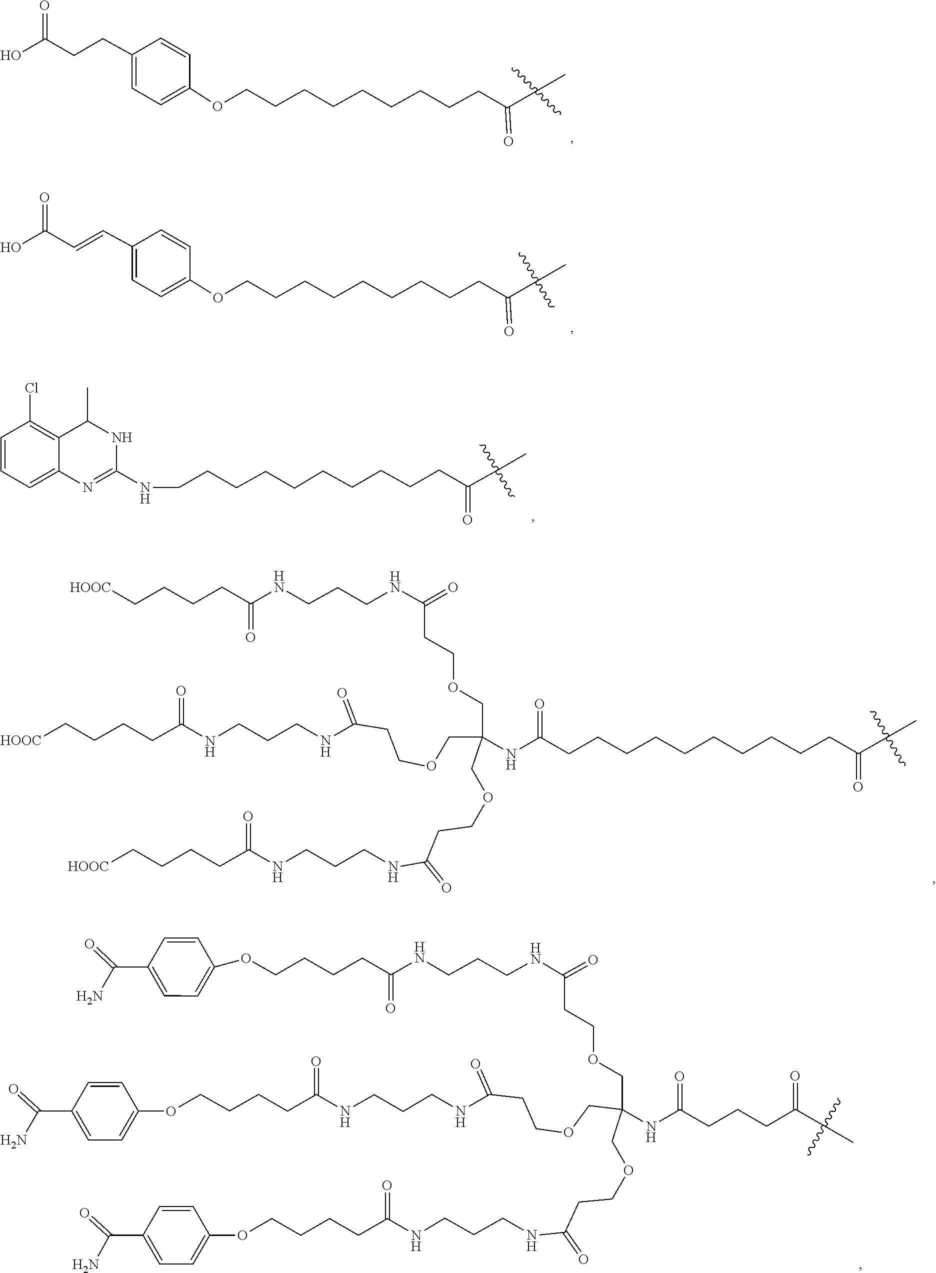 [SMILES: *C(=O)CCCCCCCCCOc1ccc(CCC(=O)O)cc1 ; *C(=O)CCCCCCCCCOc1ccc(/C=C/C(=O)O)cc1 ; *C(=O)CCCCCCCCCCNC1=Nc2cccc(Cl)c2C(C)N1 ; *C(=O)CCCCCCCCCCC(=O)NC(COCCC(=O)NCCCNC(=O)CCCCC(=O)O)(COCCC(=O)NCCCNC(=O)CCCCC(=O)O)COCCC(=O)NCCCNC(=O)CCCCC(=O)O ; *C(=O)CCCC(=O)NC(COCCC(=O)NCCCNC(=O)CCCCOc1ccc(C(N)=O)cc1)(COCCC(=O)NCCCNC(=O)CCCCOc1ccc(C(N)=O)cc1)COCCC(=O)NCCCNC(=O)CCCCOc1ccc(C(N)=O)cc1]

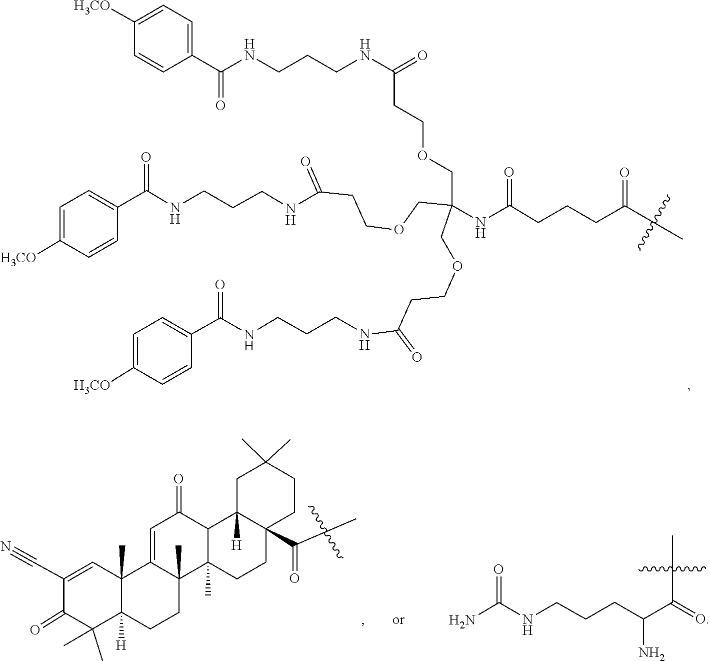

In certain embodiments, a non-negatively charged internucleotidic linkage or a neutral internucleotidic linkage is an internucleotidic linkage comprising a triazole moiety. In some embodiments, an internucleotidic linkage comprising a triazole moiety (e.g., an optionally substituted triazolyl group) has the structure of

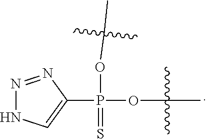 [SMILES: *OP(=S)(O*)c1c[nH]nn1]

In some embodiments, an internucleotidic linkage comprising a triazole moiety has the structure of

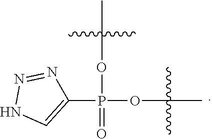 [SMILES: *OP(=O)(O*)c1c[nH]nn1]

In some embodiments, an internucleotidic linkage comprising a triazole moiety has the formula of

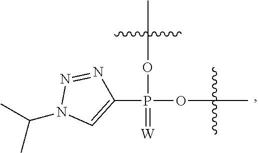

where W is O or S. In some embodiments, an internucleotidic linkage comprising an alkyne moiety (e.g., an optionally substituted alkynyl group) has the formula of

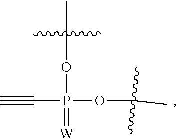

wherein W is O or S. In some embodiments, an internucleotidic linkage, e.g., a non-negatively charged internucleotidic linkage, a neutral internucleotidic linkage, comprises a cyclic guanidine moiety. In some embodiments, an internucleotidic linkage comprising a cyclic guanidine moiety has the structure of

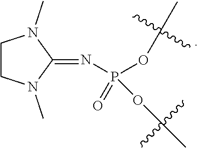 [SMILES: *OP(=O)(N=C1N(C)CCN1C)O*]

In some embodiments, a non-negatively charged internucleotidic linkage, or a neutral internucleotidic linkage, is or comprising a structure selected from

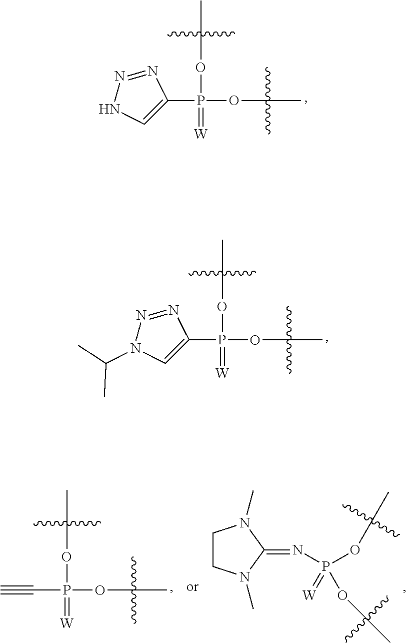

wherein W is O or S. In certain embodiments, an internucleotidic linkage, e.g., a non-negatively charged internucleotidic linkage, a neutral internucleotidic linkage, comprises a cyclic guanidine moiety. In certain embodiments, an internucleotidic linkage comprising a cyclic guanidine moiety has the structure of

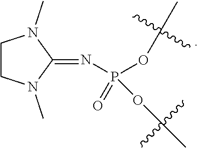 [SMILES: *OP(=O)(N=C1N(C)CCN1C)O*]

In certain embodiments, a non-negatively charged internucleotidic linkage, or a neutral internucleotidic linkage, is or comprising a structure

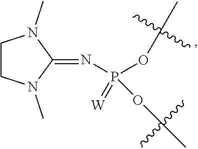

wherein W is O or S.

In certain embodiments, an internucleotidic linkage comprises a Tmg group

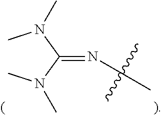 [SMILES: *N=C(N(C)C)N(C)C]

In certain embodiments, an internucleotidic linkage comprises a Tmg group and has the structure of

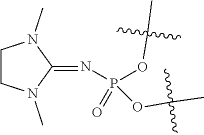 [SMILES: *OP(=O)(N=C1N(C)CCN1C)O*]

(the “Tmg internucleotidic linkage”). In certain embodiments, neutral internucleotidic linkages include internucleotidic linkages of PNA and PMO, and a Tmg internucleotidic linkage.

In certain embodiments, a non-negatively charged internucleotidic linkage has the structure of Formula I, I-a, I-b, I-c, I-n-1, I-n-2, I-n-3, I-n-4, II, II-a-1, II-a-2, II-b-1, II-b-2, II-c-1, II-c-2, II-d-1, II-d-2, etc., or a salt form thereof. In certain embodiments, a non-negatively charged internucleotidic linkage comprises an optionally substituted 3-20 membered heterocyclyl or heteroaryl group having 1-10 heteroatoms. In certain embodiments, a non-negatively charged internucleotidic linkage comprises an optionally substituted 3-20 membered heterocyclyl or heteroaryl group having 1-10 heteroatoms, wherein at least one heteroatom is nitrogen. In certain embodiments, such a heterocyclyl or heteroaryl group is of a 5-membered ring. In certain embodiments, such a heterocyclyl or heteroaryl group is of a 6-membered ring.

In certain embodiments, a non-negatively charged internucleotidic linkage comprises an optionally substituted 5-20 membered heteroaryl group having 1-10 heteroatoms. In certain embodiments, a non-negatively charged internucleotidic linkage comprises an optionally substituted 5-20 membered heteroaryl group having 1-10 heteroatoms, wherein at least one heteroatom is nitrogen. In certain embodiments, a non-negatively charged internucleotidic linkage comprises an optionally substituted 5-6 membered heteroaryl group having 1-4 heteroatoms, wherein at least one heteroatom is nitrogen. In certain embodiments, a non-negatively charged internucleotidic linkage comprises an optionally substituted 5-membered heteroaryl group having 1-4 heteroatoms, wherein at least one heteroatom is nitrogen. In certain embodiments, a heteroaryl group is directly bonded to a linkage phosphorus.

In certain embodiments, a non-negatively charged internucleotidic linkage comprises an optionally substituted 5-20 membered heterocyclyl group having 1-10 heteroatoms. In certain embodiments, a non-negatively charged internucleotidic linkage comprises an optionally substituted 5-20 membered heterocyclyl group having 1-10 heteroatoms, wherein at least one heteroatom is nitrogen. In certain embodiments, a non-negatively charged internucleotidic linkage comprises an optionally substituted 5-6 membered heterocyclyl group having 1-4 heteroatoms, wherein at least one heteroatom is nitrogen. In certain embodiments, a non-negatively charged internucleotidic linkage comprises an optionally substituted 5-membered heterocyclyl group having 1-4 heteroatoms, wherein at least one heteroatom is nitrogen. In certain embodiments, at least two heteroatoms are nitrogen. In some embodiments, a non-negatively charged internucleotidic linkage comprises an optionally substituted triazolyl group. In some embodiments, a non-negatively charged internucleotidic linkage comprises an unsubstituted triazolyl group, e.g.,

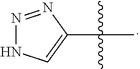 [SMILES: *c1c[nH]nn1]

In some embodiments, a non-negatively charged internucleotidic linkage comprises a substituted triazolyl group, e.g.,

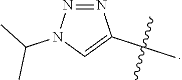 [SMILES: *c1cn(C(C)C)nn1]

In certain embodiments, a heterocyclyl group is directly bonded to a linkage phosphorus. In certain embodiments, a heterocyclyl group is bonded to a linkage phosphorus through a linker, e.g., ═N— when the heterocyclyl group is part of a guanidine moiety who directed bonded to a linkage phosphorus through its ═N—. In certain embodiments, a non-negatively charged internucleotidic linkage comprises an optionally substituted

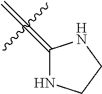 [SMILES: *=C1NCCN1]

group. In certain embodiments, a non-negatively charged internucleotidic linkage comprises a substituted

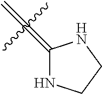 [SMILES: *=C1NCCN1]

group. In certain embodiments, a non-negatively charged internucleotidic linkage comprises a

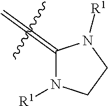

group, wherein each R¹ is independently -L-R. In certain embodiments, each R¹ is independently optionally substituted C₁₋₆ alkyl. In certain embodiments, each R¹ is independently methyl.

In certain embodiments, a modified internucleotidic linkage, e.g., a non-negatively charged internucleotidic linkage, comprises a triazole or alkyne moiety, each of which is optionally substituted. In certain embodiments, a modified internucleotidic linkage comprises a triazole moiety. In certain embodiments, a modified internucleotidic linkage comprises an unsubstituted triazole moiety. In certain embodiments, a modified internucleotidic linkage comprises a substituted triazole moiety. In certain embodiments, a modified internucleotidic linkage comprises an alkyl moiety. In certain embodiments, a modified internucleotidic linkage comprises an optionally substituted alkynyl group. In certain embodiments, a modified internucleotidic linkage comprises an unsubstituted alkynyl group. In certain embodiments, a modified internucleotidic linkage comprises a substituted alkynyl group. In certain embodiments, an alkynyl group is directly bonded to a linkage phosphorus.

In certain embodiments, a ds oligonucleotide comprises different types of internucleotidic phosphorus linkages. In certain embodiments, a chirally controlled oligonucleotide comprises at least one natural phosphate linkage and at least one modified (non-natural) internucleotidic linkage. In certain embodiments, a ds oligonucleotide comprises at least one natural phosphate linkage and at least one phosphorothioate. In certain embodiments, a ds oligonucleotide comprises at least one non-negatively charged internucleotidic linkage. In certain embodiments, a ds oligonucleotide comprises at least one natural phosphate linkage and at least one non-negatively charged internucleotidic linkage. In certain embodiments, a ds oligonucleotide comprises at least one phosphorothioate internucleotidic linkage and at least one non-negatively charged internucleotidic linkage. In certain embodiments, a ds oligonucleotide comprises at least one phosphorothioate internucleotidic linkage, at least one natural phosphate linkage, and at least one non-negatively charged internucleotidic linkage. In certain embodiments, ds oligonucleotides comprise one or more, e.g., 1-50, 1-40, 1-30, 1-20, 1-15, 1-10, 1, 2, 3, 4, 5, 6, 7, 8, 9, 10, 11, 12, 13, 14, 15, 16, 17, 18, 19, 20 or more non-negatively charged internucleotidic linkages. In certain embodiments, a non-negatively charged internucleotidic linkage is not negatively charged in that at a given pH in an aqueous solution less than 50%, 40%, 40%, 30%, 20%, 10%, 5%, or 1% of the internucleotidic linkage exists in a negatively charged salt form. In certain embodiments, a pH is about pH 7.4. In certain embodiments, a pH is about 4-9. In certain embodiments, the percentage is less than 10%. In certain embodiments, the percentage is less than 5%. In certain embodiments, the percentage is less than 1%. In certain embodiments, an internucleotidic linkage is a non-negatively charged internucleotidic linkage in that the neutral form of the internucleotidic linkage has no pKa that is no more than about 1, 2, 3, 4, 5, 6, or 7 in water. In certain embodiments, no pKa is 7 or less. In certain embodiments, no pKa is 6 or less. In certain embodiments, no pKa is 5 or less. In certain embodiments, no pKa is 4 or less. In certain embodiments, no pKa is 3 or less. In certain embodiments, no pKa is 2 or less. In certain embodiments, no pKa is 1 or less. In certain embodiments, pKa of the neutral form of an internucleotidic linkage can be represented by pKa of the neutral form of a compound having the structure of CH₃—the internucleotidic linkage-CH₃. For example, pKa of the neutral form of an internucleotidic linkage having the structure of Formula I may be represented by the pKa of the neutral form of a compound having the structure of

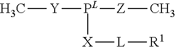

(wherein each of X, Y, Z is independently —O—, —S—, —N(R′)—; L is L^{B}, and R¹ is -L-R′), pKa of

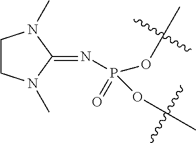 [SMILES: *OP(=O)(N=C1N(C)CCN1C)O*]

can be represented by pKa

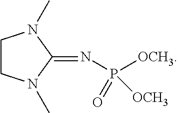

In certain embodiments, a non-negatively charged internucleotidic linkage is a neutral internucleotidic linkage. In certain embodiments, a non-negatively charged internucleotidic linkage is a positively-charged internucleotidic linkage. In certain embodiments, a non-negatively charged internucleotidic linkage comprises a guanidine moiety. In certain embodiments, a non-negatively charged internucleotidic linkage comprises a heteroaryl base moiety. In certain embodiments, a non-negatively charged internucleotidic linkage comprises a triazole moiety. In certain embodiments, a non-negatively charged internucleotidic linkage comprises an alkynyl moiety.

In certain embodiments, a neutral or non-negatively charged internucleotidic linkage has the structure of any neutral or non-negatively charged internucleotidic linkage described in any of: U.S. Pat. Nos. 9,394,333, 9,744,183, 9,605,019, 9,982,257, US 20170037399, US 20180216108, US 20180216107, U.S. Pat. No. 9,598,458, WO 2017/062862, WO 2018/067973, WO 2017/160741, WO 2017/192679, WO 2017/210647, WO 2018/098264, WO 2018/022473, WO 2018/223056, WO 2018/223073, WO 2018/223081, WO 2018/237194, WO 2019/032607, WO2019/032612, WO 2019/055951, WO 2019/075357, WO 2019/200185, WO 2019/217784, and/or WO 2019/032612,2607, WO2019032612, WO 2019/055951, WO 2019/075357, WO 2019/200185, WO 2019/217784, and/or WO 2019/032612, each neutral or non-negatively charged internucleotidic linkage of each of which is hereby incorporated by reference.

In certain embodiments, each R′ is independently optionally substituted C₁. 6 aliphatic. In certain embodiments, each R′ is independently optionally substituted C₁₋₆ alkyl. In certain embodiments, each R′ is independently —CH₃. In certain embodiments, each R⁸ is —H.

In certain embodiments, a non-negatively charged internucleotidic linkage has the structure of

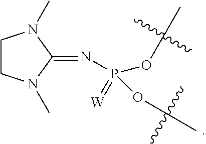

In certain embodiments, a non-negatively charged internucleotidic linkage has the structure of

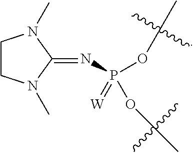

In certain embodiments, a non-negatively charged internucleotidic linkage has the structure of

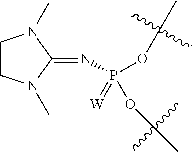

In some embodiments, a non-negatively charged internucleotidic linkage has the structure of

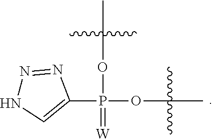

In some embodiments, a non-negatively charged internucleotidic linkage has the structure of

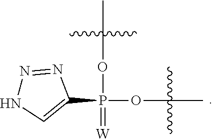

In some embodiments, a non-negatively charged internucleotidic linkage has the structure of

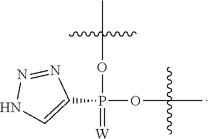

In some embodiments, anon-negatively charged internucleotidic linkage has the structure of

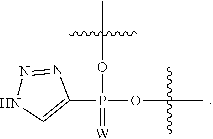

In some embodiments, a non-negatively charged internucleotidic linkage has the structure of

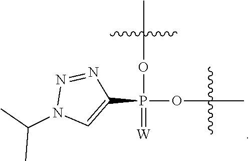

In some embodiments, a non-negatively charged internucleotidic linkage has the structure of

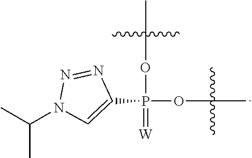

In some embodiments, a non-negatively charged internucleotidic linkage has the structure of

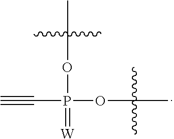

In some embodiments, a non-negatively charged internucleotidic linkage has the structure of

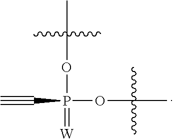

In some embodiments, a non-negatively charged internucleotidic linkage has the structure of

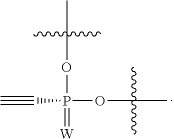

In some embodiments, W is O. In some embodiments, W is S. In some embodiments, a neutral internucleotidic linkage is a non-negatively charged internucleotidic linkage described above.

In certain embodiments, provided ds oligonucleotides comprise 1 or more internucleotidic linkages of Formula I, I-a, I-b, I-c, I-n-1, I-n-2, I-n-3, I-n-4, II, II-a-1, II-a-2, II-b-1, II-b-2, II-c-1, II-c-2, II-d-1, or II-d-2, which are described in U.S. Pat. Nos. 9,394,333, 9,744,183, 9,605,019, 9,982,257, US 20170037399, US 20180216108, US 20180216107, U.S. Pat. No. 9,598,458, WO 2017/062862, WO 2018/067973, WO 2017/160741, WO 2017/192679, WO 2017/210647, WO 2018/098264, WO 2018/022473, WO 2018/223056, WO 2018/223073, WO 2018/223081, WO 2018/237194, WO 2019/032607, WO2019/032612, WO 2019/055951, WO 2019/075357, WO 2019/200185, WO 2019/217784, and/or WO 2019/032612,2607, WO2019032612, WO 2019/055951, WO 2019/075357, WO 2019/200185, WO 2019/217784, and/or WO 2019/032612, the Formula I, I-a, I-b, I-c, I-n-1, I-n-2, I-n-3, I-n-4, II, II-a-1, II-a-2, II-b-1, II-b-2, II-c-1, II-c-2, II-d-1, or II-d-2, or salt forms thereof, each of which are independently incorporated herein by reference.

In certain embodiments, a ds oligonucleotide comprises a neutral internucleotidic linkage and a chirally controlled internucleotidic linkage. In certain embodiments, a ds oligonucleotide comprises a neutral internucleotidic linkage and a chirally controlled internucleotidic linkage which is not the neutral internucleotidic linkage. In certain embodiments, a ds oligonucleotide comprises a neutral internucleotidic linkage and a chirally controlled phosphorothioate internucleotidic linkage. In certain embodiments, the present disclosure provides a ds oligonucleotide comprising one or more non-negatively charged internucleotidic linkages and one or more phosphorothioate internucleotidic linkages, wherein each phosphorothioate internucleotidic linkage in the oligonucleotide is independently a chirally controlled internucleotidic linkage. In certain embodiments, the present disclosure provides a ds oligonucleotide comprising one or more neutral internucleotidic linkages and one or more phosphorothioate internucleotidic linkage, wherein each phosphorothioate internucleotidic linkage in the ds oligonucleotide is independently a chirally controlled internucleotidic linkage. In certain embodiments, a ds oligonucleotide comprises at least 5, 6, 7, 8, 9, 10, 11, 12, 13, 14, 15, 16, 17, 18, 19, 20 or more chirally controlled phosphorothioate internucleotidic linkages. In certain embodiments, non-negatively charged internucleotidic linkage is chirally controlled. In certain embodiments, non-negatively charged internucleotidic linkage is not chirally controlled. In certain embodiments, a neutral internucleotidic linkage is chirally controlled. In certain embodiments, a neutral internucleotidic linkage is not chirally controlled.

Without wishing to be bound by any particular theory, the present disclosure notes that a neutral internucleotidic linkage can be more hydrophobic than a phosphorothioate internucleotidic linkage (PS), which can be more hydrophobic than a natural phosphate linkage (PO). Typically, unlike a PS or PO, a neutral internucleotidic linkage bears less charge. Without wishing to be bound by any particular theory, the present disclosure notes that incorporation of one or more neutral internucleotidic linkages into a ds oligonucleotide may increase the ds oligonucleotides' ability to be taken up by a cell and/or to escape from endosomes. Without wishing to be bound by any particular theory, the present disclosure notes that incorporation of one or more neutral internucleotidic linkages can be utilized to modulate melting temperature of duplexes formed between a ds oligonucleotide and its target nucleic acid.

Without wishing to be bound by any particular theory, the present disclosure notes that incorporation of one or more non-negatively charged internucleotidic linkages, e.g., neutral internucleotidic linkages, into a ds oligonucleotide may be able to increase the ds oligonucleotide's ability to mediate a function such as target adenosine editing.

As appreciated by those skilled in the art, internucleotidic linkages such as natural phosphate linkages and those of Formula I, I-a, I-b, I-c, I-n-1, I-n-2, I-n-3, I-n-4, II, II-a-1, II-a-2, II-b-1, II-b-2, II-c-1, II-c-2, II-d-1, II-d-2, or salt forms thereof typically connect two nucleosides (which can either be natural or modified) as described in U.S. Pat. Nos. 9,394,333, 9,744,183, 9,605,019, 9,982,257, US 20170037399, US 20180216108, US 20180216107, U.S. Pat. No. 9,598,458, WO 2017/062862, WO 2018/067973, WO 2017/160741, WO 2017/192679, WO 2017/210647, WO 2018/098264, WO 2018/022473, WO 2018/223056, WO 2018/223073, WO 2018/223081, WO 2018/237194, WO 2019/032607, WO2019032612, WO 2019/055951, WO 2019/075357, WO 2019/200185, WO 2019/217784, and/or WO 2019/032612, the Formula I, I-a, I-b, I-c, I-n-1, I-n-2, I-n-3, I-n-4, II, II-a-1, II-a-2, II-b-1, II-b-2, II-c-1, II-c-2, II-d-1, II-d-2, or salt forms thereof, each of which are independently incorporated herein by reference. A typical connection, as in natural DNA and RNA, is that an internucleotidic linkage forms bonds with two sugars (which can be either unmodified or modified as described herein). In many embodiments, as exemplified herein an internucleotidic linkage forms bonds through its oxygen atoms or heteroatoms (e.g., Y and Z in various formulae) with one optionally modified ribose or deoxyribose at its 5′ carbon, and the other optionally modified ribose or deoxyribose at its 3′ carbon. In certain embodiments, each nucleoside units connected by an internucleotidic linkage independently comprises a nucleobase which is independently an optionally substituted A, T, C, G, or U, or a substituted tautomer of A, T, C, G or U, or a nucleobase comprising an optionally substituted heterocyclyl and/or a heteroaryl ring having at least one nitrogen atom.

In some embodiments, a linkage has the structure of or comprises —Y—P^{L}(—X—R^{L})—Z—, or a salt form thereof, wherein:
P^{L} is P, P(═W), P—>B(-L_{L}-R^{L})₃, or P^{N};
W is O, N(-L^{L}-R^{L}), S or Se;
P^{N} is P═N—C(-L^{L}-R′)(=L^{N}-R′) or P═N-L^{L}-R^{L};
L^{N} is ═N-L^{L1}-, ═CH-L^{L1}- wherein CH is optionally substituted, or ═N⁺(R′)(Q⁻)-L^{L1}-;
Q⁻ is an anion;
each of X, Y and Z is independently —O—, —S—, -L^{L}-N(-L^{L}-R^{L})-L^{L}, -L^{L}-N=C(-L^{L}-R^{L})-L^{L}-, or L^{L};
each R^{L} is independently-L^{L}-N(R′)₂, -L^{L}-R′, —N═C(-L^{L}-R′)₂, -L^{L}-N(R′)C(NR′)N(R′)₂, -L^{L}-N(R′)C(O)N(R′)₂, a carbohydrate, or one or more additional chemical moieties optionally connected through a linker;
each of L^{L1} and L^{L} is independently L;
-Cy^{IL}- is -Cy-;
each L is independently a covalent bond, or a bivalent, optionally substituted, linear or branched group selected from a C₁₋₃₀ aliphatic group and a C₁₋₃₀ heteroaliphatic group having 1-10 heteroatoms, wherein one or more methylene units are optionally and independently replaced by an optionally substituted group selected from C₁₋₆ alkylene, C₁₋₆ alkenylene, —C≡C—, a bivalent C1-C₆ heteroaliphatic group having 1-5 heteroatoms, —C(R′)₂—, -Cy-, —O—, —S—, —S—S—, —N(R′)—, —C(O)—, —C(S)—, —C(NR′)—, —C(NR′)N(R′)—, —N(R′)C(NR′)N(R′)—, —C(O)N(R′)—, —N(R′)C(O)N(R′)—, —N(R′)C(O)O—, —S(O)—, —S(O)₂—, —S(O)₂N(R′)—, —C(O)S—, —C(O)O—, —P(O)(OR′)—, —P(O)(SR′)—, —P(O)(R′)—, —P(O)(NR′)—, —P(S)(OR′)—, —P(S)(SR′)—, —P(S)(R′)—, —P(S)(NR′)—, —P(R′)—, —P(OR′)—, —P(SR′)—, —P(NR′)—, —P(OR′)[B(R′)₃]—, —OP(O)(OR′)O—, —OP(O)(SR′)O—, —OP(O)(R′)O—, —OP(O)(NR′)O—, —OP(OR′)O—, —OP(SR′)O—, —OP(NR′)O—, —OP(R′)O—, —OP(OR′)[B(R′)₃]O—, and —[C(R′)₂C(R′)₂O]n-, wherein n is 1-50, and one or more nitrogen or carbon atoms are optionally and independently replaced with Cy^{L};
each -Cy- is independently an optionally substituted bivalent 3-30 membered, monocyclic, bicyclic or polycyclic ring having 0-10 heteroatoms;
each Cy^{L} is independently an optionally substituted trivalent or tetravalent, 3-30 membered, monocyclic, bicyclic or polycyclic ring having 0-10 heteroatoms;
each R′ is independently —R, —C(O)R, —C(O)N(R)₂, —C(O)OR, or —S(O)₂R;
each R is independently —H, or an optionally substituted group selected from C₁₋₃₀ aliphatic, C₁₋₃₀ heteroaliphatic having 1-10 heteroatoms, C₆₋₃₀ aryl, C₆₋₃₀ arylaliphatic, C₆₋₃₀ arylheteroaliphatic having 1-10 heteroatoms, 5-30 membered heteroaryl having 1-10 heteroatoms, and 3-30 membered heterocyclyl having 1-10 heteroatoms, or
two R groups are optionally and independently taken together to form a covalent bond, or:
two or more R groups on the same atom are optionally and independently taken together with the atom to form an optionally substituted, 3-30 membered, monocyclic, bicyclic or polycyclic ring having, in addition to the atom, 0-10 heteroatoms; or
two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms.

In some embodiments, an internucleotidic linkage has the structure of —O—P^{L}(—X—R^{L})—O—, wherein each variable is independently as described herein. In some embodiments, an internucleotidic linkage has the structure of —O—P(═W)(—X—R^{L})—O— wherein each variable is independently as described herein. In some embodiments, an internucleotidic linkage has the structure of —O—P(═W)[—N(-L_{L}-R^{L})—R^{L}]—O—, wherein each variable is independently as described herein. In some embodiments, an internucleotidic linkage has the structure of —O—P(═W)(—NH-L^{L}-R^{L})—O—, wherein each variable is independently as described herein. In some embodiments, an internucleotidic linkage has the structure of —O—P(═W)[—N(R′)₂]—O—, wherein each variable is independently as described herein. In some embodiments, an internucleotidic linkage has the structure of —O—P(═W)(—NHR′)—O—, wherein each variable is independently as described herein. In some embodiments, an internucleotidic linkage has the structure of —O—P(═W)(—NHSO₂R)—O—, wherein each variable is independently as described herein. In some embodiments, an internucleotidic linkage has the structure of —O—P(═W)[—N═C(-L_{L}-R′)₂]—O—, wherein each variable is independently as described herein. In some embodiments, an internucleotidic linkage has the structure of —O—P(═W)[—N═C[N(R′)₂]₂]—O—, wherein each variable is independently as described herein. In some embodiments, an internucleotidic linkage has the structure of —OP(═W)(—N═C(R″)₂)—O—, wherein each variable is independently as described herein. In some embodiments, an internucleotidic linkage has the structure of —OP(═W)(—N(R″)₂)—O—, wherein each variable is independently as described herein. In some embodiments, W is O. In some embodiments, W is S. In some embodiments, such an internucleotidic linkage is a non-negatively charged internucleotidic linkage. In some embodiments, such an internucleotidic linkage is a neutral internucleotidic linkage.

In some embodiments, an internucleotidic linkage has the structure of —P^{L}(—X—R^{L})—Z—, wherein each variable is independently as described herein. In some embodiments, an internucleotidic linkage has the structure of —P^{L}(—X—R^{L})—, wherein each variable is independently as described herein. In some embodiments, an internucleotidic linkage has the structure of —P(═W)(—X—R^{L})—O—, wherein each variable is independently as described herein. In some embodiments, an internucleotidic linkage has the structure of —P(═W)[—N(-L^{L}-R^{L})—R^{L}]—O—, wherein each variable is independently as described herein. In some embodiments, an internucleotidic linkage has the structure of —P(═W)(—NH-L^{L}-R^{L})—O—, wherein each variable is independently as described herein. In some embodiments, an interucleotidic linkage has the structure of —P(═W)[—N(R′)₂]—O—, wherein each variable is independently as described herein. In some embodiments, an internucleotidic linkage has the structure of —P(═W)(—NHR′)—O—, wherein each variable is independently as described herein. In some embodiments, an internucleotidic linkage has the structure of —P(═W)(—NHSO₂R)—O—, wherein each variable is independently as described herein. In some embodiments, an internucleotidic linkage has the structure of —P(═W)[—N═C(-L^{L}-R′)₂]—O—, wherein each variable is independently as described herein. In some embodiments, an internucleotidic linkage has the structure of —P(═W)[—N═C[N(R′)₂]₂]—O—, wherein each variable is independently as described herein. In some embodiments, an internucleotidic linkage has the structure of —P(═W)(—N═C(R″)₂)—O—, wherein each variable is independently as described herein. In some embodiments, an internucleotidic linkage has the structure of —P(═W)(—N(R″)₂)—O—, wherein each variable is independently as described herein. In some embodiments, W is O. In some embodiments, W is S. In some embodiments, such an interucleotidic linkage is a non-negatively charged internucleotidic linkage. In some embodiments, such an internucleotidic linkage is a neutral internucleotidic linkage. In some embodiments, P of such an internucleotidic linkage is bonded to N of a sugar.

In some embodiments, a linkage is a phosphoryl guanidine internucleotidic linkage. In some embodiments, a linkage is a thio-phosphoryl guanidine internucleotidic linkage.

In some embodiments, one or more methylene units are optionally and independently replaced with a moiety as described herein. In some embodiments, L or L^{L} is or comprises —SO₂—. In some embodiments, L or L^{L} is or comprises —SO₂N(R′)—. In some embodiments, L or L^{L} is or comprises —C(O)—. In some embodiments, L or L^{L} is or comprises —C(O)O—. In some embodiments, L or L^{L} is or comprises —C(O)N(R′)—. In some embodiments, L or L^{L} is or comprises —P(═W)(R′)—. In some embodiments, L or L^{L} is or comprises —P(═O)(R′)—. In some embodiments, L or L^{L} is or comprises —P(═S)(R′)—. In some embodiments, L or L^{L} is or comprises —P(R′)—. In some embodiments, L or L^{L} is or comprises —P(═W)(OR′)—. In some embodiments, L or L^{L} is or comprises —P(═O)(OR′)—. In some embodiments, L or L^{L} is or comprises —P(═S)(OR′)—. In some embodiments, L or L^{L} is or comprises —P(OR′)—.

In some embodiments, —X—R^{L} is —N(R′)SO₂R^{L}. In some embodiments, —X—R^{L} is-N(R′)C(O)R^{L}. In some embodiments, —X—R^{L} is-N(R′)P(═O)(R′)R^{L}.

In some embodiments, a linkage, e.g., a non-negatively charged internucleotidic linkage or neutral internucleotidic linkage, has the structure of or comprises —P(═W)(—N═C(R″)₂)—, —P(═W)(—N(R′)SO₂R″)—, —P(═W)(—N(R′)C(O)R″)—, —P(═W)(—N(R″)₂)—, —P(═W)(—N(R′)P(O)(R″)₂)—, —OP(═W)(—N═C(R″)₂)O—, —OP(═W)(—N(R′)SO₂R″)O—, —OP(═W)(—N(R′)C(O)R″)O—, —OP(═W)(—N(R″)₂)O—, —OP(═W)(—N(R′)P(O)(R″)₂)O—, —P(═W)(—N═C(R″)₂)O—, —P(═W)(—N(R′)SO₂R″)O—, —P(═W)(—N(R′)C(O)R″)O—, —P(═W)(—N(R″)₂)O—, or —P(═W)(—N(R′)P(O)(R″)₂)O—, or a salt form thereof, wherein:
W is O or S;
each R″ is independently R′, —OR′, —P(═W)(R′)₂, or —N(R′)₂;
each R′ is independently —R, —C(O)R, —C(O)N(R)₂, —C(O)OR, or —S(O)₂R;
each R is independently —H, or an optionally substituted group selected from C₁₋₃₀ aliphatic, C₁₋₃₀ heteroaliphatic having 1-10 heteroatoms, C₆₋₃₀ aryl, C₆₋₃₀ arylaliphatic, C₆₋₃₀ arylheteroaliphatic having 1-10 heteroatoms, 5-30 membered heteroaryl having 1-10 heteroatoms, and 3-30 membered heterocyclyl having 1-10 heteroatoms, or
two R groups are optionally and independently taken together to form a covalent bond, or:
two or more R groups on the same atom are optionally and independently taken together with the atom to form an optionally substituted, 3-30 membered, monocyclic, bicyclic or polycyclic ring having, in addition to the atom, 0-10 heteroatoms; or
two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms.

In some embodiments, W is O. In some embodiments, an internucleotidic linkage has the structure of —P(═O)(—N═C(R″)₂)—, —P(═O)(—N(R′)SO₂R″)—, —P(═O)(—N(R′)C(O)R″)—, —P(═O)(—N(R″)₂)—, —P(═O)(—N(R′)P(O)(R″)₂)—, —OP(═O)(—N═C(R″)₂)O—, —OP(═O)(—N(R′)SO₂R″)O—, —OP(═O)(—N(R′)C(O)R″)O—, —OP(═O)(—N(R″)₂)O—, —OP(═O)(—N(R′)P(O)(R″)₂)O—, —P(═O)(—N═C(R″)₂)O—, —P(═O)(—N(R′)SO₂R″)O—, —P(═O)(—N(R′)C(O)R″)O—, —P(═O)(—N(R″)₂)O—, or —P(═O)(—N(R′)P(O)(R″)₂)O—, or a salt form thereof. In some embodiments, an internucleotidic linkage has the structure of —P(═O)(—N═C(R″)₂)— —P(═O)(—N(R″)₂)—, —OP(═O)(—N═C(R″)₂)—O—, —OP(═O)(—N(R″)₂)—O—, —P(═O)(—N═C(R″)₂)—O— or —P(═O)(—N(R″)₂)—O— or a salt form thereof. In some embodiments, an internucleotidic linkage has the structure of —OP(═O)(—N═C(R″)₂)—O— or —OP(═O)(—N(R″)₂)—O—, or a salt form thereof. In some embodiments, an internucleotidic linkage has the structure of —OP(═O)(—N═C(R″)₂)—O—, or a salt form thereof. In some embodiments, an internucleotidic linkage has the structure of —OP(═O)(—N(R″)₂)—O—, or a salt form thereof. In some embodiments, an internucleotidic linkage has the structure of —OP(═O)(—N(R′)SO₂R″)O—, or a salt form thereof. In some embodiments, an internucleotidic linkage has the structure of —OP(═O)(—N(R′)C(O)R″)O—, or a salt form thereof. In some embodiments, an internucleotidic linkage has the structure of —OP(═O)(—N(R′)P(O)(R″)₂)O—, or a salt form thereof. In some embodiments, an internucleotidic linkage is n001.

In some embodiments, W is S. In some embodiments, an internucleotidic linkage has the structure of —P(═S)(—N═C(R″)₂)—, —P(═S)(—N(R′)SO₂R″)—, —P(═S)(—N(R′)C(O)R″)—, —P(═S)(—N(R″)₂)—, —P(═S)(—N(R′)P(O)(R″)₂)—, —OP(═S)(—N═C(R″)₂)O—, —OP(═S)(—N(R′)SO₂R″)O—, —OP(═S)(—N(R′)C(O)R″)O—, —OP(═S)(—N(R″)₂)O—, —OP(═S)(—N(R′)P(O)(R″)₂)O—, —P(═S)(—N═C(R″)₂)O—, —P(═S)(—N(R′)SO₂R″)O—, —P(═S)(—N(R′)C(O)R″)O—, —P(═S)(—N(R″)₂)O—, or —P(═S)(—N(R′)P(O)(R″)₂)O—, or a salt form thereof. In some embodiments, an internucleotidic linkage has the structure of —P(═S)(—N═C(R″)₂)— —P(═S)(—N(R″)₂)—, —OP(═S)(—N═C(R″)₂)—O—, —OP(═S)(—N(R″)₂)—O—, —P(═S)(—N═C(R″)₂)—O— or —P(═S)(—N(R″)₂)—O— or a salt form thereof. In some embodiments, an internucleotidic linkage has the structure of —OP(═S)(—N═C(R″)₂)—O— or —OP(═S)(—N(R″)₂)—O—, or a salt form thereof. In some embodiments, an internucleotidic linkage has the structure of —OP(═S)(—N═C(R″)₂)—O—, or a salt form thereof. In some embodiments, an internucleotidic linkage has the structure of —OP(═S)(—N(R″)₂)—O—, or a salt form thereof. In some embodiments, an internucleotidic linkage has the structure of —OP(═S)(—N(R′)SO₂R″)O—, or a salt form thereof. In some embodiments, an internucleotidic linkage has the structure of —OP(═S)(—N(R′)C(O)R″)O—, or a salt form thereof. In some embodiments, an internucleotidic linkage has the structure of —OP(═S)(—N(R′)P(O)(R″)₂)O—, or a salt form thereof. In some embodiments, an internucleotidic linkage is *n001.

In some embodiments, an internucleotidic linkage has the structure of —P(═O)(—N(R′)SO₂R″)—, wherein R″ is as described herein. In some embodiments, an internucleotidic linkage has the structure of —P(═S)(—N(R′)SO₂R″)—, wherein R″ is as described herein. In some embodiments, an internucleotidic linkage has the structure of —P(═O)(—N(R′)SO₂R″)O—, wherein R″ is as described herein. In some embodiments, an internucleotidic linkage has the structure of —P(═S)(—N(R′)SO₂R″)O—, wherein R″ is as described herein. In some embodiments, an internucleotidic linkage has the structure of —OP(═O)(—N(R′)SO₂R″)O—, wherein R″ is as described herein. In some embodiments, an internucleotidic linkage has the structure of —OP(═S)(—N(R′)SO₂R″)O—, wherein R″ is as described herein. In some embodiments, R′, e.g., of —N(R′)—, is hydrogen or optionally substituted C₁₋₆ aliphatic. In some embodiments, R′ is C₁₋₆ alkyl. In some embodiments, R′ is hydrogen. In some embodiments, R″, e.g., in —SO₂R″, is R′ as described herein. In some embodiments, an internucleotidic linkage has the structure of —P(═O)(—NHSO₂R″)—, wherein R″ is as described herein. In some embodiments, an internucleotidic linkage has the structure of —P(═S)(—NHSO₂R″)—, wherein R″ is as described herein. In some embodiments, an internucleotidic linkage has the structure of —P(═O)(—NHSO₂R″)O—, wherein R″ is as described herein. In some embodiments, an internucleotidic linkage has the structure of —P(═S)(—NHSO₂R″)O—, wherein R″ is as described herein. In some embodiments, an internucleotidic linkage has the structure of —OP(═O)(—NHSO₂R″)O—, wherein R″ is as described herein. In some embodiments, an internucleotidic linkage has the structure of —OP(═S)(—NHSO₂R″)O—, wherein R″ is as described herein. In some embodiments, —X—R^{L} is-N(R′)SO₂R^{L}, wherein each of R′ and R^{L} is independently as described herein. In some embodiments, R^{L} is R″. In some embodiments, R^{L} is R′. In some embodiments, —X—R^{L} is —N(R′)SO₂R″, wherein R′ is as described herein. In some embodiments, —X—R^{L} is —N(R′)SO₂R′, wherein R′ is as described herein. In some embodiments, —X—R^{L} is —NHSO₂R′, wherein R′ is as described herein. In some embodiments, R′ is R as described herein. In some embodiments, R′ is optionally substituted C₁₋₆ aliphatic. In some embodiments, R′ is optionally substituted C₁₋₆ alkyl. In some embodiments, R′ is optionally substituted phenyl. In some embodiments, R′ is optionally substituted heteroaryl. In some embodiments, R″, e.g., in —SO₂R″, is R. In some embodiments, R is an optionally substituted group selected from C₁₋₆ aliphatic, aryl, heterocyclyl, and heteroaryl. In some embodiments, R is optionally substituted C₁₋₆ aliphatic. In some embodiments, R is optionally substituted C₁₋₆ alkyl. In some embodiments, R is optionally substituted C₁₋₆ alkenyl. In some embodiments, R is optionally substituted C₁₋₆ alkynyl. In some embodiments, R is optionally substituted methyl. In some embodiments, —X—R^{L} is —NHSO₂CH₃. In some embodiments, R is —CF₃. In some embodiments, R is methyl. In some embodiments, R is optionally substituted ethyl. In some embodiments, R is ethyl. In some embodiments, R is —CH₂CHF₂. In some embodiments, R is —CH₂CH₂OCH₃. In some embodiments, R is optionally substituted propyl. In some embodiments, R is optionally substituted butyl. In some embodiments, R is n-butyl. In some embodiments, R is —(CH₂)₆NH₂. In some embodiments, R is an optionally substituted linear C₂₋₂₀ aliphatic. In some embodiments, R is optionally substituted linear C₂₋₂₀ alkyl. In some embodiments, R is linear C₂₋₂₀ alkyl. In some embodiments, R is optionally substituted C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, or C₂₀ aliphatic. In some embodiments, R is optionally substituted C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, or C₂₀ alkyl. In some embodiments, R is optionally substituted linear C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, or C₂₀ alkyl. In some embodiments, R is linear C₁, C₂, C₃, C₄, C₅, C₆, C₇, C₈, C₉, C₁₀, C₁₁, C₁₂, C₁₃, C₁₄, C₁₈, C₁₆, C₁₇, C₁₈, C₁₉, or C₂₀ alkyl. In some embodiments, R is optionally substituted phenyl. In some embodiments, R is phenyl. In some embodiments, R is p-methylphenyl. In some embodiments, R is 4-dimethylaminophenyl. In some embodiments, R is 3-pyridinyl. In some embodiments, R is

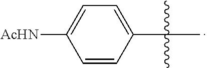 [SMILES: *c1ccc(NC(C)=O)cc1]

In some embodiments, R is

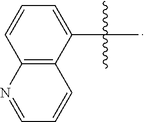 [SMILES: *c1cccc2ncccc12]

In some embodiments, R is benzyl. In some embodiments, R is optionally substituted heteroaryl. In some embodiments, R is optionally substituted 1,3-diazolyl. In some embodiments, R is optionally substituted 2-(1,3)-diazolyl. In some embodiments, R is optionally substituted 1-methyl-2-(1,3)-diazolyl. In some embodiments, R is isopropyl. In some embodiments, R″ is —N(R′)₂. In some embodiments, R″ is —N(CH₃)₂. In some embodiments, R″, e.g., in —SO₂R″, is —OR′, wherein R′ is as described herein. In some embodiments, R′ is R as described herein. In some embodiments, R″ is —OCH₃. In some embodiments, a linkage is —OP(═O)(—NHSO₂R)O—, wherein R is as described herein. In some embodiments, R is optionally substituted linear alkyl as described herein. In some embodiments, R is linear alkyl as described herein. In some embodiments, a linkage is —OP(═O)(—NHSO₂CH₃)O—. In some embodiments, a linkage is —OP(═O)(—NHSO₂CH₂CH₃)O—. In some embodiments, a linkage is —OP(═O)(—NHSO₂CH₂CH₂OCH₃)O—. In some embodiments, a linkage is —OP(═O)(—NHSO₂CH₂Ph)O—. In some embodiments, a linkage is —OP(═O)(—NHSO₂CH₂CHF₂)O—. In some embodiments, a linkage is —OP(═O)(—NHSO₂(4-methylphenyl))O—. In some embodiments, —X—R^{L} is

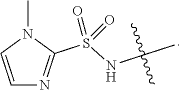 [SMILES: *NS(=O)(=O)c1nccn1C]

In some embodiments, a linkage is —OP(═O)(—X—R^{L})O—, wherein —X—R^{L} is

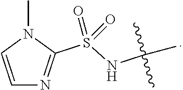 [SMILES: *NS(=O)(=O)c1nccn1C]

In some embodiments, a linkage is —OP(═O)(—NHSO₂CH(CH₃)₂)O—. In some embodiments, a linkage is —OP(═O)(—NHSO₂N(CH₃)₂)O—.

In some embodiments, an internucleotidic linkage has the structure of —P(═O)(—N(R′)C(O)R″)—, wherein R″ is as described herein. In some embodiments, an internucleotidic linkage has the structure of —P(═S)(—N(R′)C(O)R″)—, wherein R″ is as described herein. In some embodiments, an internucleotidic linkage has the structure of —P(═O)(—N(R′)C(O)R″)O—, wherein R″ is as described herein. In some embodiments, an internucleotidic linkage has the structure of —P(═S)(—N(R′)C(O)R″)O—, wherein R″ is as described herein. In some embodiments, an internucleotidic linkage has the structure of —OP(═O)(—N(R′)C(O)R″)O—, wherein R″ is as described herein. In some embodiments, an internucleotidic linkage has the structure of —OP(═S)(—N(R′)C(O)R″)O—, wherein R″ is as described herein. In some embodiments, R′, e.g., of —N(R′)—, is hydrogen or optionally substituted C₁₋₆ aliphatic. In some embodiments, R′ is C₁₋₆ alkyl. In some embodiments, R′ is hydrogen. In some embodiments, R″, e.g., in —C(O)R″, is R′ as described herein. In some embodiments, an internucleotidic linkage has the structure of —P(═O)(—NHC(O)R″)—, wherein R″ is as described herein. In some embodiments, an internucleotidic linkage has the structure of —P(═S)(—NHC(O)R″)—, wherein R″ is as described herein. In some embodiments, an internucleotidic linkage has the structure of —P(═O)(—NHC(O)R″)O—, wherein R″ is as described herein. In some embodiments, an internucleotidic linkage has the structure of —P(═S)(—NHC(O)R″)O—, wherein R″ is as described herein. In some embodiments, an internucleotidic linkage has the structure of —OP(═O)(—NHC(O)R″)O—, wherein R″ is as described herein. In some embodiments, an internucleotidic linkage has the structure of —OP(═S)(—NHC(O)R″)O—, wherein R″ is as described herein. In some embodiments, —X—R^{L} is-N(R′)COR^{L}, wherein R^{L} is as described herein. In some embodiments, —X—R^{L} is —N(R′)COR″, wherein R″ is as described herein. In some embodiments, —X—R^{L} is —N(R′)COR′, wherein R′ is as described herein. In some embodiments, —X—R^{L} is —NHCOR′, wherein R′ is as described herein. In some embodiments, R′ is R as described herein. In some embodiments, R′ is optionally substituted C₁₋₆ aliphatic. In some embodiments, R′ is optionally substituted C₁₋₆ alkyl. In some embodiments, R′ is optionally substituted phenyl. In some embodiments, R′ is optionally substituted heteroaryl. In some embodiments, R″, e.g., in —C(O)R″, is R. In some embodiments, R is an optionally substituted group selected from C₁₋₆ aliphatic, aryl, heterocyclyl, and heteroaryl. In some embodiments, R is optionally substituted C₁₋₆ aliphatic. In some embodiments, R is optionally substituted C₁₋₆ alkyl. In some embodiments, R is optionally substituted C₁₋₆ alkenyl. In some embodiments, R is optionally substituted C₁₋₆ alkynyl. In some embodiments, R is methyl. In some embodiments, —X—R^{L} is —NHC(O)CH₃. In some embodiments, R is optionally substituted methyl. In some embodiments, R is —CF₃. In some embodiments, R is optionally substituted ethyl. In some embodiments, R is ethyl. In some embodiments, R is —CH₂CHF₂. In some embodiments, R is —CH₂CH₂OCH₃. In some embodiments, R is optionally substituted C₁₋₂₀ (e.g., C₁₋₆, C₂₋₆, C₃₋₆, C₁₋₁₀, C₂₋₁₀, C₃₋₁₀, C₂₋₂₀, C₃₋₂₀, C₁₀₋₂₀, 1, 2, 3, 4, 5, 6, 7, 8, 9, 10, 11, 12, 13, 14, 15, 16, 17, 18, 19, or 20, etc.) aliphatic. In some embodiments, R is optionally substituted C₁₋₂₀ (e.g., C₁₋₆, C₂₋₆, C₃₋₆, C₁₋₁₀, C₂₋₁₀, C₃₋₁₀, C₂₋₂₀, C₃₋₂₀, C₁₀₋₂₀, 1, 2, 3, 4, 5, 6, 7, 8, 9, 10, 11, 12, 13, 14, 15, 16, 17, 18, 19, or 20, etc.) alkyl. In some embodiments, R is an optionally substituted linear C₂₋₂₀ aliphatic. In some embodiments, R is optionally substituted linear C₂₋₂₀ alkyl. In some embodiments, R is linear C₂₋₂₀ alkyl. In some embodiments, R is optionally substituted C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, or C₂₀ aliphatic. In some embodiments, R is optionally substituted C₁, C₂, C₃, C₄, C₅, C₆, C₇, C₈, C₉, C₁₀, C₁₁, C₁₂, C₁₃, C₁₄, C₁₈, C₁₆, C₁₇, C₁₈, C₁₉, or C₂₀ alkyl. In some embodiments, R is optionally substituted linear C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, or C₂₀ alkyl. In some embodiments, R is linear C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, or C₂₀ alkyl. In some embodiments, R is optionally substituted aryl. In some embodiments, R is optionally substituted phenyl. In some embodiments, R is p-methylphenyl. In some embodiments, R is benzyl. In some embodiments, R is optionally substituted heteroaryl. In some embodiments, R is optionally substituted 1,3-diazolyl. In some embodiments, R is optionally substituted 2-(1,3)-diazolyl. In some embodiments, R is optionally substituted 1-methyl-2-(1,3)-diazolyl. In some embodiments, R^{L} is —(CH₂)₅NH₂. In some embodiments, R^{L} is

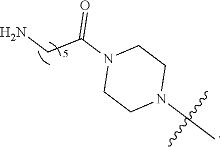

In some embodiments, R^{L} is

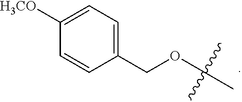 [SMILES: *OCc1ccc(OC)cc1]

In some embodiments, R″ is —N(R′)₂. In some embodiments, R″ is —N(CH₃)₂. In some embodiments, —X—R^{L} is —N(R′)CON(R^{L})₂, wherein each of R′ and R^{L} is independently as described herein. In some embodiments, —X—R^{L} is —NHCON(R^{L})₂, wherein R^{L} is as described herein. In some embodiments, two R′ or two R^{L} are taken together with the nitrogen atom to which they are attached to form a ring as described herein, e.g., optionally substituted C

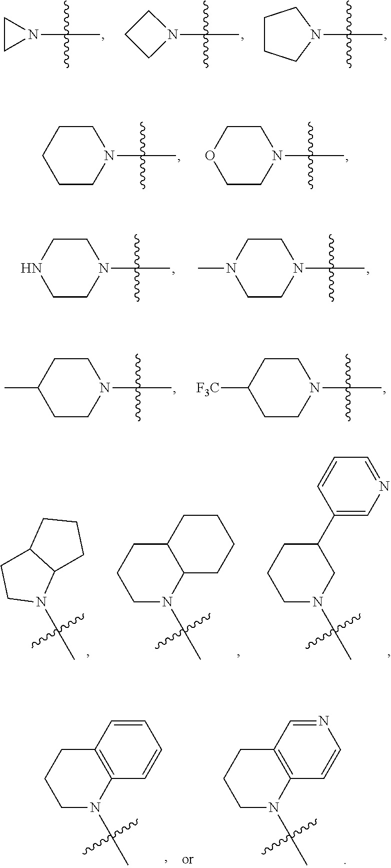

In some embodiments, R″, e.g., in —C(O)R″, is —OR′, wherein R′ is as described herein. In some embodiments, R′ is R as described herein. In some embodiments, is optionally substituted C₁₋₆ aliphatic. In some embodiments, is optionally substituted C₁₋₆ alkyl. In some embodiments, R″ is —OCH₃. In some embodiments, —X—R^{L} is —N(R′)C(O)OR^{L}, wherein each of R′ and R^{L} is independently as described herein. In some embodiments, R is

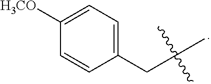 [SMILES: *Cc1ccc(OC)cc1]

In some embodiments, —X—R^{L} is —NHC(O)OCH₃. In some embodiments, —X—R^{L} is —NHC(O)N(CH₃)₂. In some embodiments, a linkage is —OP(O)(NHC(O)CH₃)O—. In some embodiments, a linkage is —OP(O)(NHC(O)OCH₃)O—. In some embodiments, a linkage is —OP(O)(NHC(O)(p-methylphenyl))O—. In some embodiments, a linkage is —OP(O)(NHC(O)N(CH₃)₂)O—. In some embodiments, —X—R^{L} is —N(R′)R^{L}, wherein each of R′ and R^{L} is independently as described herein. In some embodiments, —X—R^{L} is N(R′)R^{L}, wherein each of R′ and R^{L} is independently not hydrogen. In some embodiments, —X—R^{L} is —NHR^{L}, wherein R^{L} is as described herein. In some embodiments, R^{L} is not hydrogen. In some embodiments, R^{L} is optionally substituted aryl or heteroaryl. In some embodiments, R^{L} is optionally substituted aryl. In some embodiments, R^{L} is optionally substituted phenyl. In some embodiments, —X—R^{L} is —N(R′)₂, wherein each R′ is independently as described herein. In some embodiments, —X—R^{L} is NHR′, wherein R′ is as described herein. In some embodiments, —X—R^{L} is —NHR, wherein R is as described herein. In some embodiments, —X—R^{L} is R^{L}, wherein R^{L} is as described herein. In some embodiments, R^{L} is —N(R′)₂, wherein each R′ is independently as described herein. In some embodiments, R^{L} is —NHR′, wherein R′ is as described herein. In some embodiments, R^{L} is —NHR, wherein R is as described herein. In some embodiments, R^{L} is —N(R′)₂, wherein each R′ is independently as described herein. In some embodiments, none of R′ in —N(R′)₂ is hydrogen. In some embodiments, R^{L} is —N(R′)₂, wherein each R′ is independently C₁₋₆ aliphatic. In some embodiments, R^{L} is -L-R′, wherein each of L and R′ is independently as described herein. In some embodiments, R^{L} is -L-R, wherein each of L and R is independently as described herein. In some embodiments, R^{L} is —N(R′)-Cy-N(R′)—R′. In some embodiments, R^{L} is —N(R′)-Cy-C(O)—R′. In some embodiments, R^{L} is —N(R′)-Cy-O—R′. In some embodiments, R^{L} is —N(R′)-Cy-SO₂—R′. In some embodiments, R^{L} is —N(R′)-Cy-SO₂—N(R′)₂. In some embodiments, R^{L} is —N(R′)-Cy-C(O)—N(R′)₂. In some embodiments, R^{L} is —N(R′)-Cy-OP(O)(R″)₂. In some embodiments, -Cy- is an optionally substituted bivalent aryl group. In some embodiments, -Cy- is optionally substituted phenylene. In some embodiments, -Cy- is optionally substituted 1,4-phenylene. In some embodiments, -Cy- is 1,4-phenylene. In some embodiments, R^{L} is —N(CH₃)₂. In some embodiments, R^{L} is —N(i-Pr)₂. In some embodiments, R^{L} is

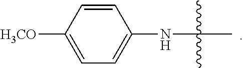 [SMILES: *Nc1ccc(OC)cc1]

In some embodiments, R^{L} is

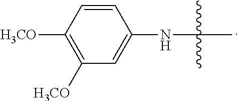 [SMILES: *Nc1ccc(OC)c(OC)c1]

In some embodiments, R^{L} is

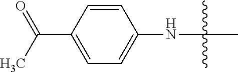 [SMILES: *Nc1ccc(C(C)=O)cc1]

In some embodiments, R^{L} is

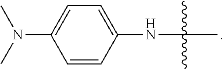 [SMILES: *Nc1ccc(N(C)C)cc1]

In some embodiments, R^{L} is

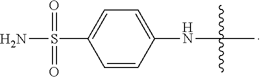 [SMILES: *Nc1ccc(S(N)(=O)=O)cc1]

In some embodiments, R^{L} is

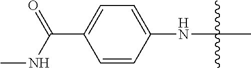 [SMILES: *Nc1ccc(C(=O)NC)cc1]

In some embodiments, R^{L} is

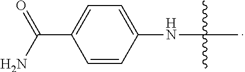 [SMILES: *Nc1ccc(C(N)=O)cc1]

In some embodiments, R^{L} is

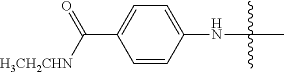

In some embodiments, R^{L} is

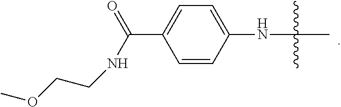 [SMILES: *Nc1ccc(C(=O)NCCOC)cc1]

In some embodiments, R^{L} is

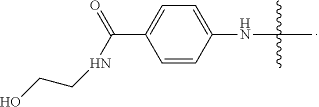 [SMILES: *Nc1ccc(C(=O)NCCO)cc1]

In some embodiments, R^{L} is

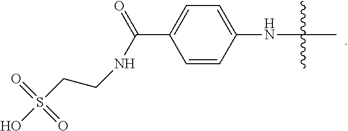 [SMILES: *Nc1ccc(C(=O)NCCS(=O)(=O)O)cc1]

In some embodiments, R^{L} is

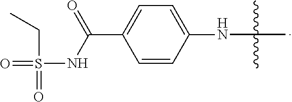 [SMILES: *Nc1ccc(C(=O)NS(=O)(=O)CC)cc1 ; "."]

In some embodiments, R^{L} is

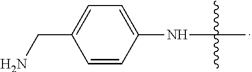 [SMILES: *Nc1ccc(CN)cc1 ; "."]

In some embodiments, R^{L} is

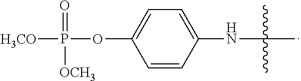 [SMILES: *Nc1ccc(OP(=O)(OC)OC)cc1 ; "."]

In some embodiments, R^{L} is

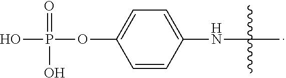 [SMILES: *Nc1ccc(OP(=O)(O)O)cc1 ; "."]

In some embodiments, R^{L} is

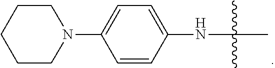 [SMILES: *Nc1ccc(N2CCCCC2)cc1]

In some embodiments, R^{L} is

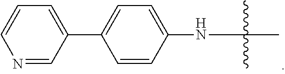 [SMILES: *Nc1ccc(-c2cccnc2)cc1]

In some embodiments. R^{L} is

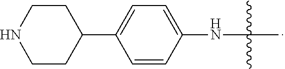 [SMILES: *Nc1ccc(C2CCNCC2)cc1 ; "."]

In some embodiments, R^{L} is

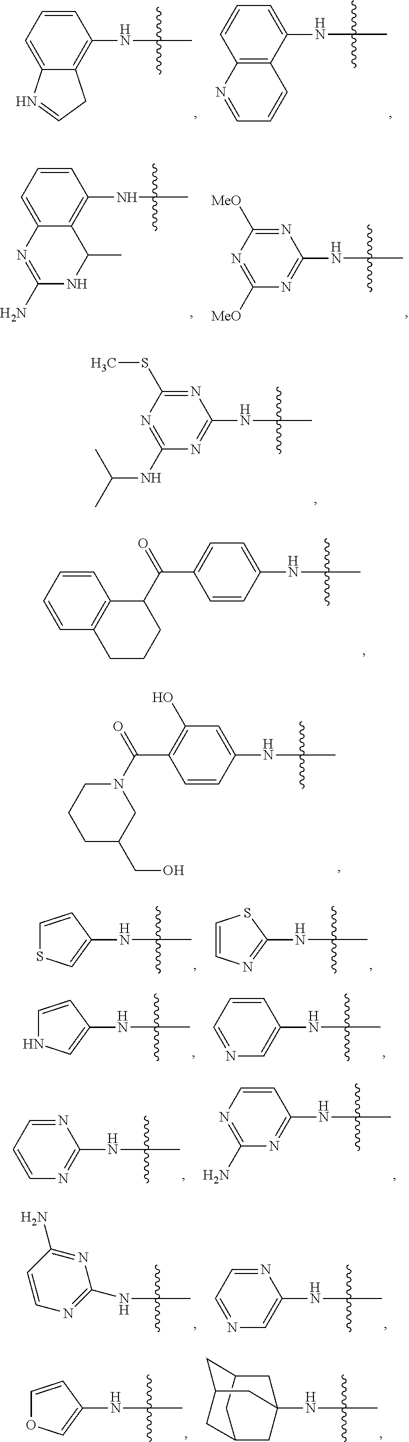

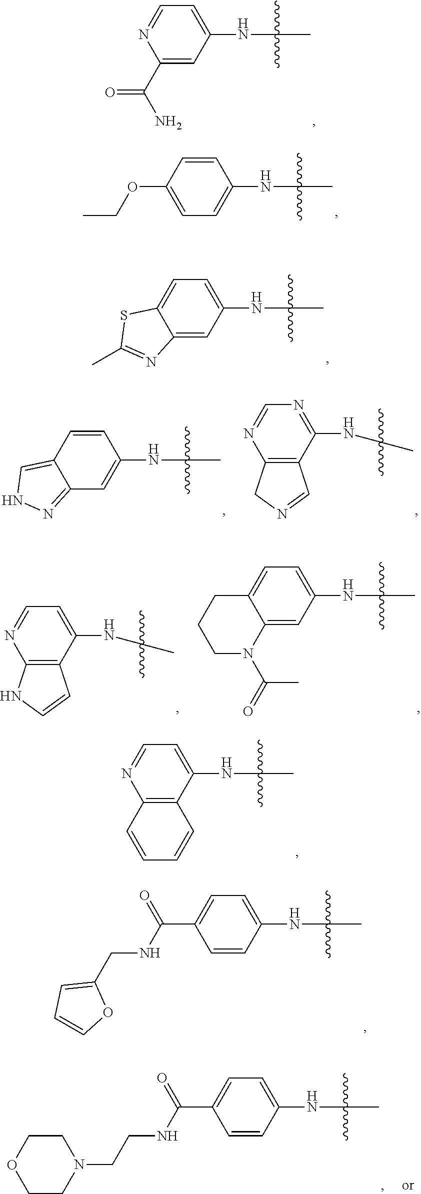

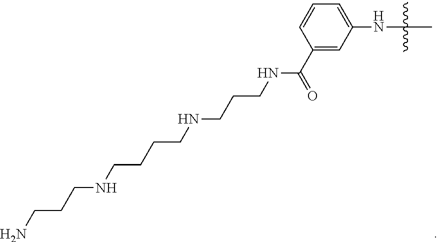 [SMILES: *Nc1cccc(C(=O)NCCCNCCCCNCCCN)c1]

In some embodiments, —X—R^{L} is —N(R′)—C(O)-Cy-R^{L}. In some embodiments, —X—R^{L} is R^{L}. In some embodiments, R^{L} is —N(R′)—C(O)-Cy-O—R′. In some embodiments, R^{L} is —N(R′)—C(O)-Cy-R′. In some embodiments, R^{L} is —N(R′)—C(O)-Cy-C(O)—R′. In some embodiments, R^{L} is —N(R′)—C(O)-Cy-N(R′)₂. In some embodiments, R^{L} is —N(R′)—C(O)-Cy-SO₂—N(R′)₂. In some embodiments, R^{L} is N(R′)—C(O)-Cy-C(O)—N(R′)₂. In some embodiments, R^{L} is —N(R′)—C(O)-Cy-C(O)—N(R′)—SO₂—R′. In some embodiments, R′ is R as described herein. In some embodiments, R^{L} is

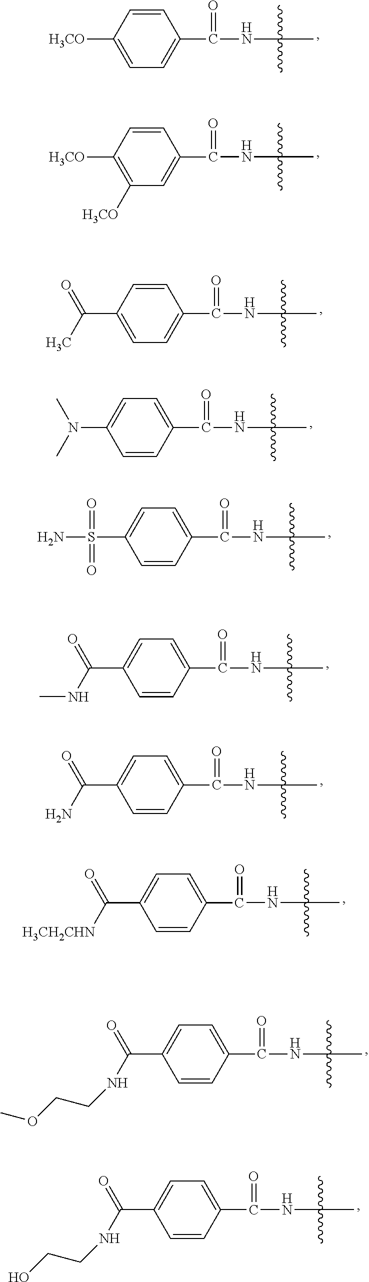

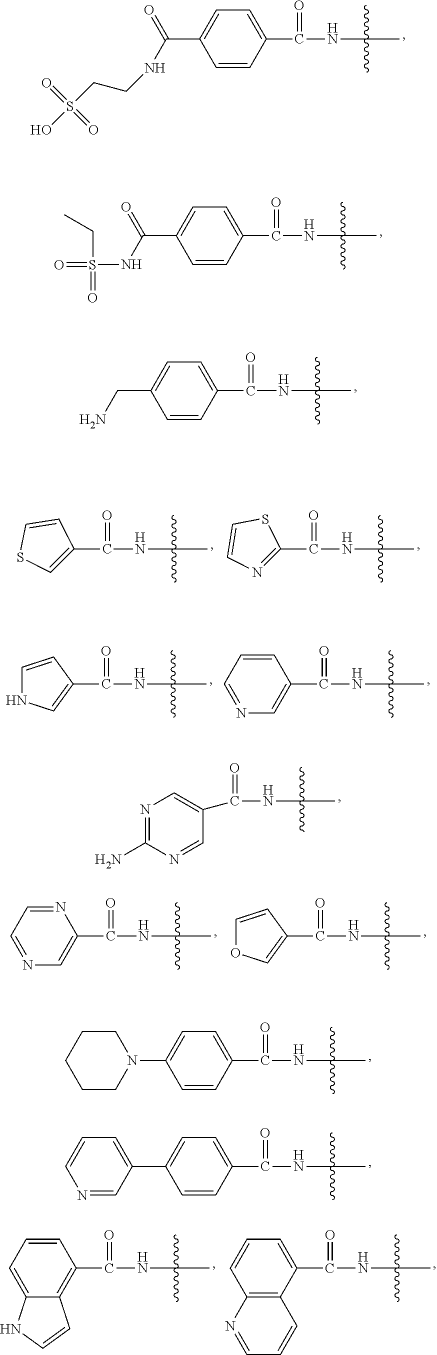

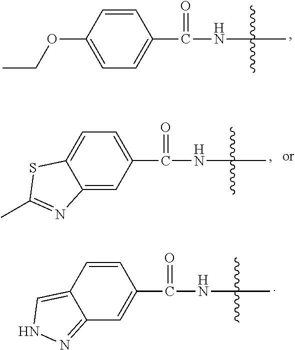 [SMILES: *NC(=O)c1ccc(OCC)cc1 ; *NC(=O)c1ccc2sc(C)nc2c1 ; *NC(=O)c1ccc2c[nH]nc2c1]

As described herein, in some embodiments, one or more methylene units of L, or a variable which comprises or is L, are independently replaced with —O—, —N(R′)—, —C(O)—, —C(O)N(R′)—, —SO₂—, —SO₂N(R′)—, or -Cy-. In some embodiments, a methylene unit is replaced with -Cy-. In some embodiments, -Cy- is an optionally substituted bivalent aryl group. In some embodiments, -Cy- is optionally substituted phenylene. In some embodiments, -Cy- is optionally substituted 1,4-phenylene. In some embodiments, -Cy- is an optionally substituted bivalent 5-20 (e.g. 5, 6, 7, 8, 9, 10, 11, 12, 13, 14, 15, 16, 17, 18, 19, or 20) membered heteroaryl group having 1-10 (e.g., 1, 2, 3, 4, 5, 6, 7, 8, 9, or 10) heteroatoms. In some embodiments, -Cy- is monocyclic. In some embodiments, -Cy- is bicyclic. In some embodiments, -Cy- is polycyclic. In some embodiments, each monocyclic unit in -Cy- is independently 3-10 (e.g., 3, 4, 5, 6, 7, 8, 9, or 10) membered, and is independently saturated, partially saturated, or aromatic. In some embodiments, -Cy- is an optionally substituted 3-20 (e.g., 3, 4, 5, 6, 7, 8, 9, 10, 11, 12, 13, 14, 15, 16, 17, 18, 19, or 20) membered monocyclic, bicyclic or polycyclic aliphatic group. In some embodiments, -Cy- is an optionally substituted 3-20 (e.g., 3, 4, 5, 6, 7, 8, 9, 10, 11, 12, 13, 14, 15, 16, 17, 18, 19, or 20) membered monocyclic, bicyclic or polycyclic heteroaliphatic group having 1-10 (e.g., 1, 2, 3, 4, 5, 6, 7, 8, 9, or 10) heteroatoms.

In some embodiments, an internucleotidic linkage has the structure of —P(═O)(—N(R′)P(O)(R″)₂)—, wherein each R″ is independently as described herein. In some embodiments, an internucleotidic linkage has the structure of —P(═S)(—N(R′)P(O)(R″)₂)—, wherein each R″ is independently as described herein. In some embodiments, an internucleotidic linkage has the structure of —P(═O)(—N(R′)P(O)(R″)₂)O—, wherein each R″ is independently as described herein. In some embodiments, an internucleotidic linkage has the structure of —P(═S)(—N(R′)P(O)(R″)₂)O—, wherein each R″ is independently as described herein. In some embodiments, an internucleotidic linkage has the structure of —OP(═O)(—N(R′)P(O)(R″)₂)O—, wherein each R″ is independently as described herein. In some embodiments, an internucleotidic linkage has the structure of —OP(═S)(—N(R′)P(O)(R″)₂)O—, wherein each R″ is independently as described herein. In some embodiments, R′, e.g., of —N(R′)—, is hydrogen or optionally substituted C₁₋₆ aliphatic. In some embodiments, R′ is C₁₋₆ alkyl. In some embodiments, R′ is hydrogen. In some embodiments, R″, e.g., in —P(O)(R″)₂, is R′ as described herein. In some embodiments, an internucleotidic linkage has the structure of —P(═O)(—NHP(O)(R″)₂)—, wherein each R″ is independently as described herein. In some embodiments, an internucleotidic linkage has the structure of —P(═S)(—NHP(O)(R″)₂)—, wherein each R″ is independently as described herein. In some embodiments, an internucleotidic linkage has the structure of —P(═O)(—NHP(O)(R″)₂)O—, wherein each R″ is independently as described herein. In some embodiments, an internucleotidic linkage has the structure of —P(═S)(—NHP(O)(R″)₂)O—, wherein each R″ is independently as described herein. In some embodiments, an internucleotidic linkage has the structure of —OP(═O)(—NHP(O)(R″)₂)O—, wherein each R″ is independently as described herein. In some embodiments, an internucleotidic linkage has the structure of —OP(═S)(—NHP(O)(R″)₂)O—, wherein each R″ is independently as described herein. In some embodiments, an occurrence of R″, e.g., in —P(O)(R″)₂, is R. In some embodiments, R is an optionally substituted group selected from C₁₋₆ aliphatic, aryl, heterocyclyl, and heteroaryl. In some embodiments, R is optionally substituted C₁₋₆ aliphatic. In some embodiments, R is optionally substituted C₁₋₆ alkyl. In some embodiments, R is optionally substituted C₁₋₆ alkenyl. In some embodiments, R is optionally substituted C₁₋₆ alkynyl. In some embodiments, R is methyl. In some embodiments, R is optionally substituted methyl. In some embodiments, R is —CF₃. In some embodiments, R is optionally substituted ethyl. In some embodiments, R is ethyl. In some embodiments, R is —CH₂CHF₂. In some embodiments, R is —CH₂CH₂OCH₃. In some embodiments, R is optionally substituted C₁₋₂₀ (e.g., C₁₋₆, C₂₋₆, C₃₋₆, C₁₋₁₀, C₂₋₁₀, C₃₋₁₀, C₂₋₂₀, C₃₋₂₀, C₁₀₋₂₀, 1, 2, 3, 4, 5, 6, 7, 8, 9, 10, 11, 12, 13, 14, 15, 16, 17, 18, 19, or 20, etc.) aliphatic. In some embodiments, R is optionally substituted C₁₋₂₀ (e.g., C₁₋₆, C₂₋₆, C₃₋₆, C₁₋₁₀, C₂₋₁₀, C₃₋₁₀, C₂₋₂₀, C₃₋₂₀, C₁₀₋₂₀, 1, 2, 3, 4, 5, 6, 7, 8, 9, 10, 11, 12, 13, 14, 15, 16, 17, 18, 19, or 20, etc.) alkyl. In some embodiments, R is an optionally substituted linear C₂₋₂₀ aliphatic. In some embodiments, R is optionally substituted linear C₂₋₂₀ alkyl. In some embodiments, R is linear C₂₋₂₀ alkyl. In some embodiments, R is isopropyl. In some embodiments, R is optionally substituted C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, or C₂₀ aliphatic. In some embodiments, R is optionally substituted C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, or C₂₀ alkyl. In some embodiments, R is optionally substituted linear C₁, C₂, C₃, C₄, C₅, C₆, C₇, C₈, C₉, C₁₀, C₁₁, C₁₂, C₁₃, C₁₄, C₁₈, C₁₆, C₁₇, C₁₈, C₁₉, or C₂₀ alkyl. In some embodiments, R is linear C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, or C₂₀ alkyl. In some embodiments, each R″ is independently R as described herein, for example, in some embodiments, each R″ is methyl. In some embodiments, R″ is optionally substituted aryl. In some embodiments, R is optionally substituted phenyl. In some embodiments, R is p-methylphenyl. In some embodiments, R is benzyl. In some embodiments, R is optionally substituted heteroaryl. In some embodiments, R is optionally substituted 1,3-diazolyl. In some embodiments, R is optionally substituted 2-(1,3)-diazolyl. In some embodiments, R is optionally substituted 1-methyl-2-(1,3)-diazolyl. In some embodiments, an occurrence of R″ is —N(R′)₂. In some embodiments, R″ is —N(CH₃)₂. In some embodiments, an occurrence of R″, e.g., in —P(O)(R″)₂, is —OR′, wherein R′ is as described herein. In some embodiments, R′ is R as described herein. In some embodiments, is optionally substituted C₁₋₆ aliphatic. In some embodiments, is optionally substituted C₁₋₆ alkyl. In some embodiments, R″ is —OCH₃. In some embodiments, each R″ is —OR′ as described herein. In some embodiments, each R″ is —OCH₃. In some embodiments, each R″ is —OH. In some embodiments, a linkage is —OP(O)(NHP(O)(OH)₂)O—. In some embodiments, a linkage is —OP(O)(NHP(O)(OCH₃)₂)O—. In some embodiments, a linkage is —OP(O)(NHP(O)(CH₃)₂)O—.

In some embodiments, —N(R″)₂ is —N(R′)₂. In some embodiments, —N(R″)₂ is —NHR. In some embodiments, —N(R″)₂ is —NHC(O)R. In some embodiments, —N(R″)₂ is —NHC(O)OR. In some embodiments, —N(R″)₂ is —NHS(O)₂R.

In some embodiments, an internucleotidic linkage is a phosphoryl guanidine internucleotidic linkage. In some embodiments, an internucleotidic linkage comprises —X—R^{L} as described herein. In some embodiments, —X—R^{L} is-N=C(-L^{L}-R^{L})₂. In some embodiments, —X—R^{L} is-N=C[N(R^{L})₂]₂. In some embodiments, —X—R^{L} is-N=C[NR′R^{L}]₂. In some embodiments, —X—R^{L} is —N═C[N(R′)₂]₂. In some embodiments, —X—R^{L} is —N═C[N(R^{L})₂](CHR^{L1}R^{L2}), wherein each of R^{L1} and R^{L2} is independently as described herein. In some embodiments, —X—R^{L} is-N=C(NR′R^{L})(CHR^{L1}R^{L2}), wherein each of R^{L1} and R^{L2} is independently as described herein. In some embodiments, —X—R^{L} is —N═C(NR′R^{L})(CR′R^{L1}R^{L2}), wherein each of R^{L1} and R^{L2} is independently as described herein. In some embodiments, —X—R^{L} is —N=C[N(R′)₂](CHR′R^{L2}). In some embodiments, —X—R^{L} is —N=C[N(R^{L})₂](R^{L}). In some embodiments, —X—R^{L} is —N=C(NR′R^{L})(R^{L}). In some embodiments, —X—R^{L} is —N=C(NR′R^{L})(R′). In some embodiments, —X—R^{L} is —N═C[N(R′)₂](R′). In some embodiments, —X—R^{L} is —N=C(NR′R^{L})(NR′R^{L2}), wherein each R^{L1} and R^{L2} is independently R^{L}, and each R′ and R^{L} is independently as described herein. In some embodiments, —X—R^{L} is —N=C(NR′R^{L1})(NR′R^{L2}), wherein variable is independently as described herein. In some embodiments, —X—R^{L} is —N═C(NR′R^{L1})(CHR′R^{L2}), wherein variable is independently as described herein. In some embodiments, —X—R^{L} is N=C(NR′R^{L1})(R′), wherein variable is independently as described herein. In some embodiments, each R′ is independently R. In some embodiments, R is optionally substituted C₁₋₆ aliphatic. In some embodiments, R is methyl. In some embodiments, —X—R^{L} is

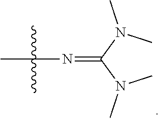 [SMILES: *N=C(N(C)C)N(C)C]

In some embodiments, two groups selected from R′, R^{L}, R^{L1}, R^{L2}, etc. (in some embodiments, on the same atom (e.g., —N(R′)₂, or —NR′R^{L}, or —N(R^{L})₂, wherein R′ and R^{L} can independently be R as described herein), etc.), or on different atoms (e.g., the two R′ in —N═C(NR′R^{L})(CR′R^{L1}R^{L2}) or —N═C(NR′R^{L1})(NR′R^{L2}); can also be two other variables that can be R, e.g., R^{L}, R^{L}, R^{L2} etc.)) are independently R and are taken together with their intervening atoms to form a ring as described herein. In some embodiments, two of R, R′, R^{L}, R^{L1}, or R^{L2} on the same atom, e.g., of —N(R′)₂, —N(R^{L})₂, —NR′R^{L}, —NR′R^{L}, —NR′R^{L2}, —CR′R^{L1}R^{L2}, etc., are taken together to form a ring as described herein. In some embodiments, two R′, R^{L}, R^{L1}, or R^{L2} on two different atoms, e.g., the two R′ in —N═C(NR′R^{L})(CR′R^{L1}R^{L2}), —N═C(NR′R^{L1})(NR′R^{L2}), etc. are taken together to form a ring as described herein. In some embodiments, a formed ring is an optionally substituted 3-20 (e.g., 3-15, 3-12, 3-10, 3-9, 3-8, 3-7, 3-6, 4-15, 4-12, 4-10, 4-9, 4-8, 4-7, 4-6, 5-15, 5-12, 5-10, 5-9, 5-8, 5-7, 5-6, 1, 2, 3, 4, 5, 6, 7, 8, 9, 10, 11, 12, 13, 14, 15, 16, 17, 18, 19, 20, etc.) monocyclic, bicyclic or tricyclic ring having 0-5 additional heteroatoms. In some embodiments, a formed ring is monocyclic as described herein. In some embodiments, a formed ring is an optionally substituted 5-10 membered monocyclic ring. In some embodiments, a formed ring is bicyclic. In some embodiments, a formed ring is polycyclic. In some embodiments, two groups that are or can be R (e.g., the two R′ in —N═C(NR′R^{L})(CR′R^{L1}R^{L2}) or —N═C(NR′RY)(NR′R^{L2}), the two R′ in —N═C(NR′R^{L})(CR′R^{L1}R^{L2}), —N═C(NR′R^{L1})(NR′R^{L2}), etc.) are taken together to form an optionally substituted bivalent hydrocarbon chain, e.g., an optionally substituted C₁₋₂₀ aliphatic chain, optionally substituted —(CH₂)n- wherein n is 1-20 (e.g., 1, 2, 3, 4, 5, 6, 7, 8, 9, 10, 11, 12, 13, 14, 15, 16, 17, 18, 19, or 20). In some embodiments, a hydrocarbon chain is saturated. In some embodiments, a hydrocarbon chain is partially unsaturated. In some embodiments, a hydrocarbon chain is unsaturated. In some embodiments, two groups that are or can be R (e.g., the two R′ in —N═C(NR′R^{L})(CR′R^{L1}R^{L2}) or —N═C(NR′R^{L1})(NR′R^{L2}), the two R′ in —N═C(NR′R^{L})(CR′R^{L1}R^{L2}), —N═C(NR′R^{L1})(NR′R^{L2}), etc.) are taken together to form an optionally substituted bivalent heteroaliphatic chain, e.g., an optionally substituted C₁₋₂₀ heteroaliphatic chain having 1-10 heteroatoms. In some embodiments, a heteroaliphatic chain is saturated. In some embodiments, a heteroaliphatic chain is partially unsaturated. In some embodiments, a heteroaliphatic chain is unsaturated. In some embodiments, a chain is optionally substituted —(CH₂)—. In some embodiments, a chain is optionally substituted —(CH₂)₂—. In some embodiments, a chain is optionally substituted —(CH₂)—. In some embodiments, a chain is optionally substituted —(CH₂)₂—. In some embodiments, a chain is optionally substituted —(CH₂)₃—. In some embodiments, a chain is optionally substituted —(CH₂)₄—. In some embodiments, a chain is optionally substituted —(CH₂)₅—. In some embodiments, a chain is optionally substituted —(CH₂)₆—. In some embodiments, a chain is optionally substituted —CH═CH—. In some embodiments, a chain is optionally substituted

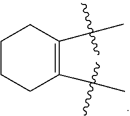 [SMILES: *C1=C(*)CCCC1]

In some embodiments, a chain is optionally substituted

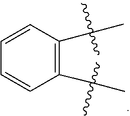 [SMILES: *c1ccccc1*]

In some embodiments, a chain is optionally substituted

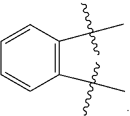 [SMILES: *c1ccccc1*]

In some embodiments, a chain is optionally substituted

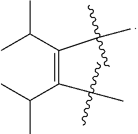 [SMILES: */C(=C(\*)C(C)C)C(C)C]

In some embodiments, a chain is optionally substituted

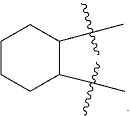 [SMILES: *C1CCCCC1*]

In some embodiments, a chain is optionally substituted

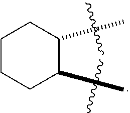

In some embodiments, a chain is optionally substituted

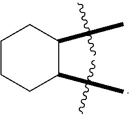

In some embodiments, a chain is optionally substituted

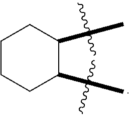

In some embodiments, a chain is optionally substituted

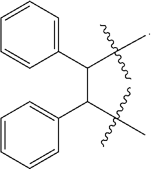 [SMILES: *C(c1ccccc1)C(*)c1ccccc1]

In some embodiments, two of R, R′, R^{L}, R^{L1}, R^{L2}, etc. on different atoms are taken together to form a ring as described herein. For examples, in some embodiments, —X—R^{L} is

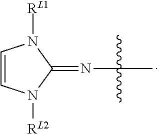

In some embodiments, —X—R^{L} is

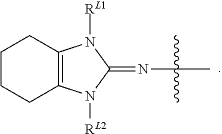

In some embodiments, —X—R^{L} is

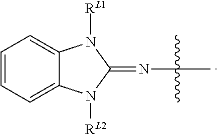

In some embodiments, —X—R^{L} is

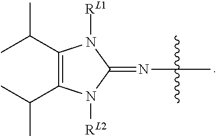

In some embodiments, —X—R^{L} is

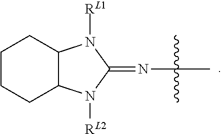

In some embodiments, —X—R^{L} is

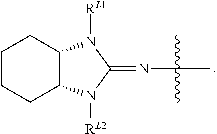

In some embodiments, —X—R^{L} is

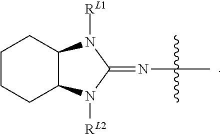

In some embodiments, —X—R^{L} is R^{L2} is

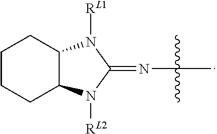

In some embodiments, —X—R^{L} is

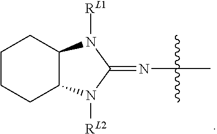

In some embodiments, —X—R^{L} is

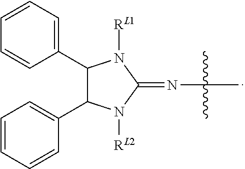

In some embodiments, —N(R′)₂, —N(R)₂, —N(R^{L})₂, —NR′R^{L}, —NR′^{L1}, —NR′R^{L2}, NR^{L1}R^{L2}, etc. is a formed ring. In some embodiments, a ring is optionally substituted

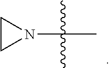 [SMILES: *N1CC1]

In some embodiments, a ring is optionally substituted

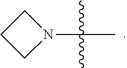 [SMILES: *N1CCC1]

In some embodiments, a ring is optionally substituted

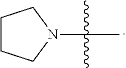 [SMILES: *N1CCCC1]

In some embodiments, a ring is optionally substituted

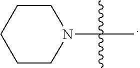 [SMILES: *N1CCCCC1]

In some embodiments, a ring is optionally substituted

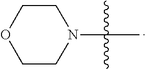 [SMILES: *N1CCOCC1]

In some embodiments, a ring is optionally substituted

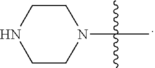 [SMILES: *N1CCNCC1]

In some embodiments, a ring is optionally substituted

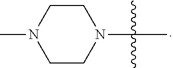 [SMILES: *N1CCN(C)CC1]

In some embodiments, a ring is optionally substituted

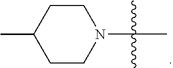 [SMILES: *N1CCC(C)CC1]

In some embodiments, a ring is optionally substituted

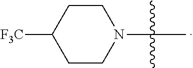 [SMILES: *N1CCC(C(F)(F)F)CC1]

In some embodiments, a ring is optionally substituted

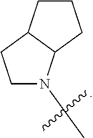 [SMILES: *N1CCC2CCCC21]

In some embodiments, a ring is optionally substituted

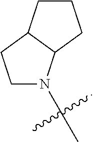 [SMILES: *N1CCC2CCCC21]

In some embodiments, a ring is optionally substituted

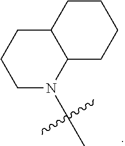 [SMILES: *N1CCCC2CCCCC21]

In some embodiments, a ring is optionally substituted

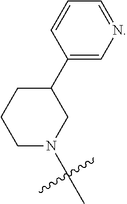 [SMILES: *N1CCCC(c2cccnc2)C1]

In some embodiments, a ring is optionally substituted

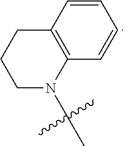 [SMILES: *N1CCCc2ccccc21]

In some embodiments, a ring is optionally substituted

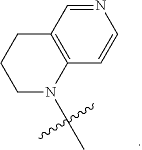 [SMILES: *N1CCCc2cnccc21]

In some embodiments, R^{L1} and R^{L2} are the same. In some embodiments, R^{L1} and R^{L2} are different. In some embodiments, each of R^{L1} and R^{L2} is independently R^{L} as described herein, e.g., below.

In some embodiments, R^{L} is optionally substituted C₁₋₃₀ aliphatic. In some embodiments, R^{L} is optionally substituted C₁₋₃₀ alkyl. In some embodiments, R^{L} is linear. In some embodiments, R^{L} is optionally substituted linear C₁₋₃₀ alkyl. In some embodiments, R^{L} is optionally substituted C₁₋₆ alkyl. In some embodiments, R^{L} is methyl. In some embodiments, R^{L} is ethyl. In some embodiments, R^{L} is n-propyl. In some embodiments, R^{L} is isopropyl. In some embodiments, R^{L} is n-butyl. In some embodiments, R^{L} is tert-butyl. In some embodiments, R^{L} is (E)-CH₂—CH═CH—CH₂—CH₃. In some embodiments R^{L} is (Z)—CH₂—CH═CH—CH₂—CH₃. In some embodiments, R^{L} is

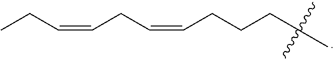 [SMILES: *CCC/C=C\C/C=C\CC]

In some embodiments, R^{L} is

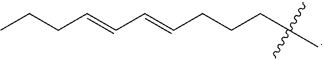 [SMILES: *CCC/C=C/C=C/CCC]

In some embodiments, R^{L} is CH₃(CH₂)₂C≡CC≡C(CH₂)₃—. In some embodiments, R^{L} is CH₃(CH₂)₅C≡C—. In some embodiments, R^{L} optionally substituted aryl. In some embodiments, R^{L} is optionally substituted phenyl. In some embodiments, R^{L} is phenyl substituted with one or more halogen. In some embodiments, R^{L} is phenyl optionally substituted with halogen, —N(R′), or —N(R′)C(O)R′. In some embodiments, R^{L} is phenyl optionally substituted with —C₁, —Br, —F, —N(Me)₂, or —NHCOCH₃. In some embodiments, R^{L} is LLR′, wherein L^{L} is an optionally substituted C₁₋₂₀ saturated, partially unsaturated or unsaturated hydrocarbon chain. In some embodiments, such a hydrocarbon chain is linear. In some embodiments, such a hydrocarbon chain is unsubstituted. In some embodiments, L^{L} is (E)-CH₂—CH═CH—. In some embodiments, L^{L} is —CH₂—C≡C—CH₂—. In some embodiments, L^{L} is —(CH₂)₃—. In some embodiments, L^{L} is —(CH₂)₄—. In some embodiments, L^{L} is —(CH₂)r-, wherein n is 1-30 (e.g., 1-20, 5-30, 6-30, 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30, etc.). In some embodiments, R′ is optionally substituted aryl as described herein. In some embodiments, R′ is optionally substituted phenyl. In some embodiments, R′ is phenyl. In some embodiments, R′ is optionally substituted heteroaryl as described herein. In some embodiments, R′ is 2′-pyridinyl. In some embodiments, R′ is 3′-pyridinyl. In some embodiments, R^{L} is

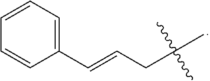 [SMILES: *C/C=C/c1ccccc1]

In some embodiments, R^{L} is

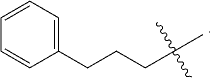 [SMILES: *CCCc1ccccc1]

In some embodiments, R^{L} is

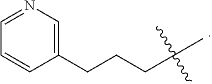 [SMILES: *CCCc1cccnc1]

In some embodiments, R^{L} is-L^{L}-N(R′)₂, wherein each variable is independently as described herein. In some embodiments, each R′ is independently C₁₋₆ aliphatic as described herein. In some embodiments, —N(R′)₂ is —N(CH₃)₂. In some embodiments, —N(R′)₂ is —NH₂. In some embodiments, R^{L} is —(CH₂)r-N(R′)₂, wherein n is 1-30 (e.g., 1-20, 5-30, 6-30, 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30, etc.). In some embodiments, R^{L} is —(CH₂CH₂O)ₙ—CH₂CH₂—N(R′)₂, wherein n is 1-30 (e.g., 1-20, 5-30, 6-30, 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30, etc.). In some embodiments, R^{L} is

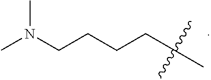 [SMILES: *CCCCN(C)C]

In some embodiments, R^{L} is

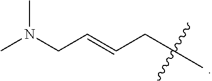 [SMILES: *C/C=C/CN(C)C]

In some embodiments, R^{L} is

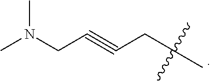 [SMILES: *CC#CCN(C)C]

In some embodiments, R^{L} is —(CH₂)ₙ—NH₂. In some embodiments, R^{L} is —(CH₂CH₂O)r-CH₂CH₂—NH₂. In some embodiments, R^{L} is —(CH₂CH₂O)ₙ—CH₂CH₂—R′, wherein n is 1-30 (e.g., 1-20, 5-30, 6-30, 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30, etc.). In some embodiments, R^{L} is —(CH₂CH₂O)ₙₙ—CH₂CH₂CH₃, wherein n is 1-30 (e.g., 1-20, 5-30, 6-30, 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30, etc.). In some embodiments, R^{L} is —(CH₂CH₂O)ₙ—CH₂CH₂OH, wherein n is 1-30 (e.g., 1-20, 5-30, 6-30, 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30, etc.). In some embodiments, R^{L} is or comprises a carbohydrate moiety, e.g., GalNAc. In some embodiments, R^{L} is -L^{L}-GalNAc. In some embodiments, R^{L} is

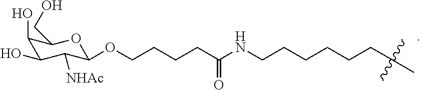 [SMILES: *CCCCCCNC(=O)CCCCOC1OC(CO)C(O)C(O)C1NC(C)=O]

In some embodiments, one or more methylene units of L^{L} are independently replaced with -Cy- (e.g., optionally substituted 1,4-phenylene, a 3-30 membered bivalent optionally substituted monocyclic, bicyclic, or polycyclic cycloaliphatic ring, etc.), —O—, —N(R′)— (e.g., —NH), —C(O)—, —C(O)N(R′)— (e.g., —C(O)NH—), —C(NR′)— (e.g., —C(NH)—), —N(R′)C(O)(N(R′)— (e.g., —NHC(O)NH—), —N(R′)C(NR′)(N(R′)— (e.g., —NHC(NH)NH—), —(CH₂CH₂O)ₙ—, etc. For example, in some embodiments, R^{L} is

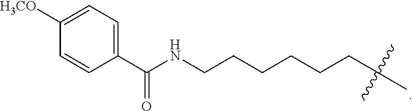 [SMILES: *CCCCCCNC(=O)c1ccc(OC)cc1]

In some embodiments, R^{L} is

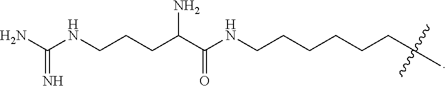 [SMILES: *CCCCCCNC(=O)C(N)CCCNC(=N)N]

In some embodiments, R^{L} is

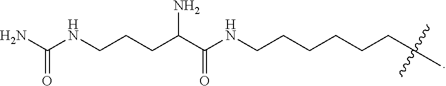 [SMILES: *CCCCCCNC(=O)C(N)CCCNC(N)=O]

In some embodiments, R^{L} is

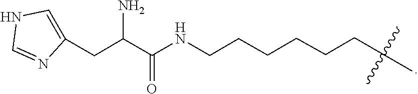 [SMILES: *CCCCCCNC(=O)C(N)Cc1c[nH]cn1]

In some embodiments, R^{L} is

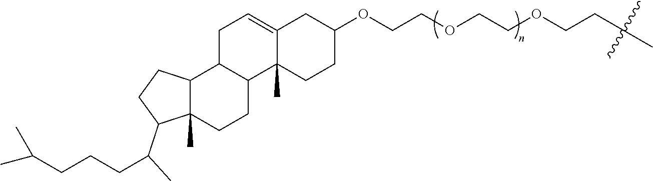

wherein n is 0-20. In some embodiments, R^{L} is or comprises one or more additional chemical moieties (e.g., carbohydrate moieties, GalNAc moieties, etc.) optionally substituted connected through a linker (which can be bivalent or polyvalent). For example, in some embodiments, R^{L} is

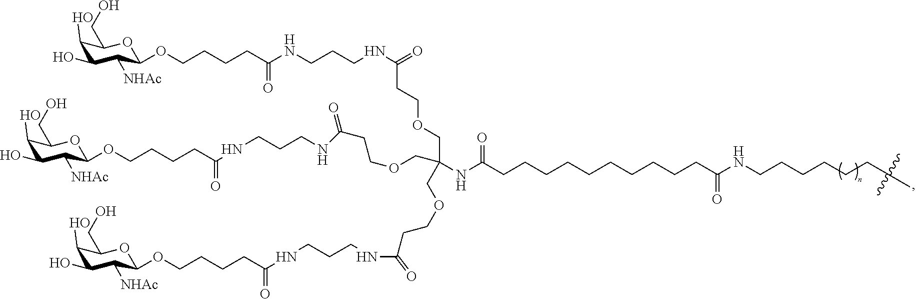

wherein n is 0-20. In some embodiments, R^{L} is

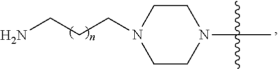

wherein n is 0-20. In some embodiments, R^{L} is R′ as described herein. As described herein, many variable can independently be R′. In some embodiments, R′ is R as described herein. As described herein, various variables can independently be R. In some embodiments, R is optionally substituted C₁₋₆ aliphatic. In some embodiments, R is optionally substituted C₁₋₆ alkyl. In some embodiments, R is methyl. In some embodiments, R is optionally substituted cycloaliphatic. In some embodiments, R is optionally substituted cycloalkyl. In some embodiments, R is optionally substituted aryl. In some embodiments, R is optionally substituted phenyl. In some embodiments, R is optionally substituted heteroaryl. In some embodiments, R is optionally substituted heterocyclyl. In some embodiments, R is optionally substituted C₁₋₂₀ heterocyclyl having 1-5 heteroatoms, e.g., one of which is nitrogen. In some embodiments, R is optionally substituted

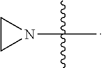 [SMILES: *N1CC1]

In some embodiments, R is optionally substituted

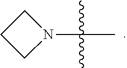 [SMILES: *N1CCC1]

In some embodiments, R is optionally substituted

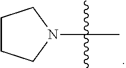 [SMILES: *N1CCCC1]

In some embodiments, R is optionally substituted

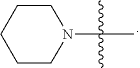 [SMILES: *N1CCCCC1]

In some embodiments, R is optionally substituted

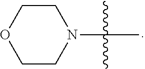 [SMILES: *N1CCOCC1]

In some embodiments, R is optionally substituted

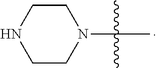 [SMILES: *N1CCNCC1]

In some embodiments, R is optionally substituted

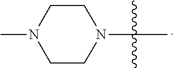 [SMILES: *N1CCN(C)CC1]

In some embodiments, R is optionally substituted

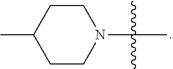 [SMILES: *N1CCC(C)CC1]

In some embodiments, R is optionally substituted

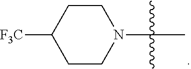 [SMILES: *N1CCC(C(F)(F)F)CC1]

In some embodiments, R is optionally substituted

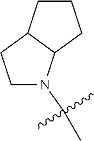 [SMILES: *N1CCC2CCCC21]

In some embodiments, R is optionally substituted

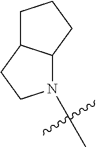 [SMILES: *N1CCC2CCCC21]

In some embodiments, R is optionally substituted

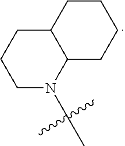 [SMILES: *N1CCCC2CCCCC21]

In some embodiments, R is optionally substituted

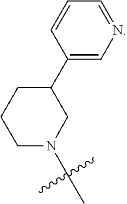 [SMILES: *N1CCCC(c2cccnc2)C1]

In some embodiments, R is optionally substituted

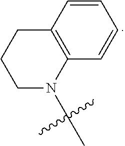 [SMILES: *N1CCCc2ccccc21]

In some embodiments, R is optionally substituted

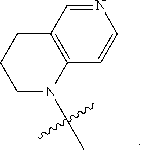 [SMILES: *N1CCCc2cnccc21]

In some embodiments, —X—R^{L} is

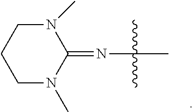 [SMILES: *N=C1N(C)CCCN1C]

In some embodiments, —X—R^{L} is

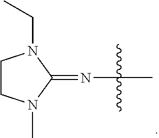 [SMILES: *N=C1N(C)CCN1CC]

In some embodiments, —X—R^{L} is

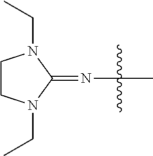 [SMILES: *N=C1N(CC)CCN1CC]

In some embodiments, —X—R^{L} is

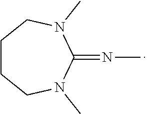

In some embodiments, —X—R^{L} is

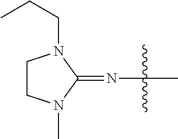 [SMILES: *N=C1N(C)CCN1CCC]

In some embodiments, —X—R^{L} is

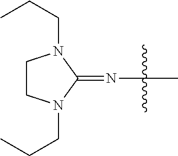 [SMILES: *N=C1N(CCC)CCN1CCC]

In some embodiments, —X—R^{L} is

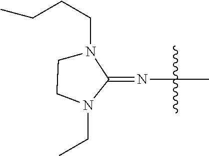 [SMILES: *N=C1N(CC)CCN1CCCC]

In some embodiments, —X—R^{L} is

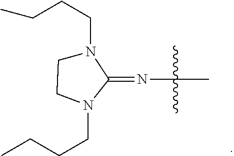 [SMILES: *N=C1N(CCCC)CCN1CCCC]

In some embodiments, —X—R^{L} is

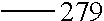

In some embodiments, —X—R^{L} is

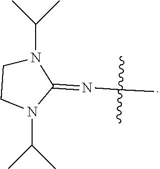 [SMILES: *N=C1N(C(C)C)CCN1C(C)C]

In some embodiments, —X—R^{L} is

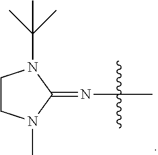 [SMILES: *N=C1N(C)CCN1C(C)(C)C]

In some embodiments, —X—R^{L} is

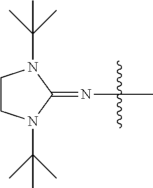 [SMILES: *N=C1N(C(C)(C)C)CCN1C(C)(C)C]

In some embodiments, —X—R^{L} is

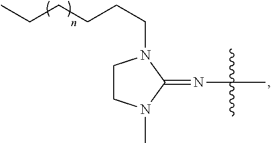

wherein n is 1-20. In some embodiments, —X—R^{L} is

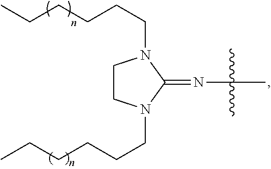

wherein n is 1-20. In some embodiments, —X—R^{L} is selected from:

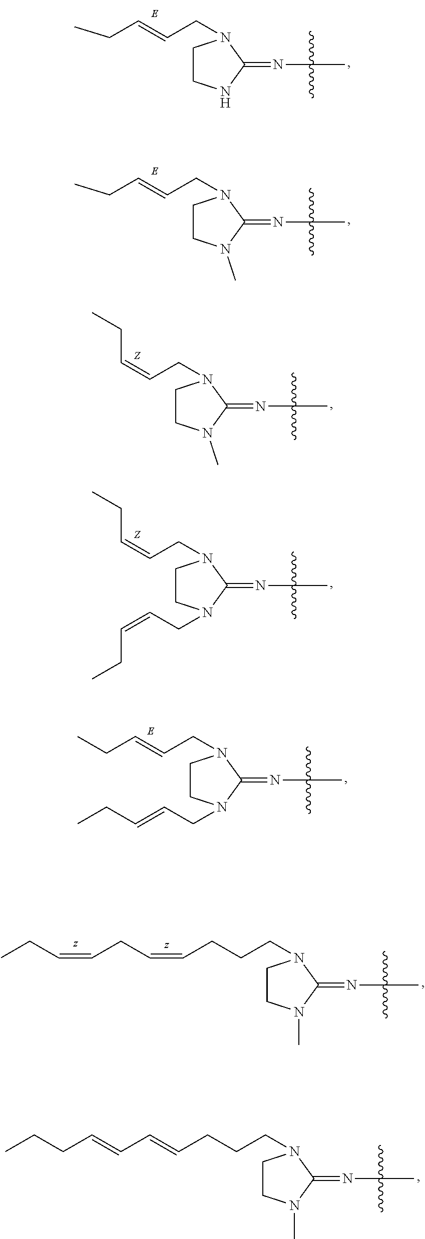

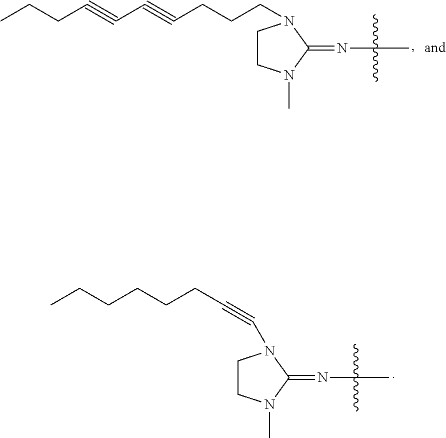 [SMILES: *N=C1N(C)CCN1CCCC#CC#CCCC ; *N=C1N(C)CCN1C#CCCCCCC]

In some embodiments, —X—R^{L} is

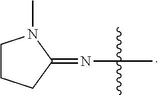 [SMILES: *N=C1CCCN1C]

In some embodiments, —X—R^{L} is

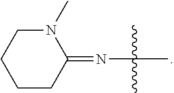 [SMILES: *N=C1CCCCN1C]

In some embodiments, —X—R^{L} is

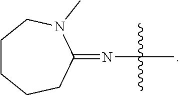 [SMILES: *N=C1CCCCCN1C]

In some embodiments, R^{L} is R″ as described herein. In some embodiments, R^{L} is R as described herein.

In some embodiments, R″ or R^{L} is or comprises an additional chemical moiety. In some embodiments, R″ or R^{L} is or comprises an additional chemical moiety, wherein the additional chemical moiety is or comprises a carbohydrate moiety. In some embodiments, R″ or R^{L} is or comprises a GalNAc. In some embodiments, R^{L} or R″ is replaced with, or is utilized to connect to, an additional chemical moiety.

In some embodiments, X is —O—. In some embodiments, X is —S—. In some embodiments, X is -L^{L}-N(-L^{L}-R^{L})-L^{L}-. In some embodiments, X is —N(-L^{L}-R^{L})L^{L} In some embodiments, X is -L^{L}-N(-L^{L}-R^{L})—. In some embodiments, X is —N(-L^{L}-R^{L})—. In some embodiments, X is -L^{L}-N=C(-L^{L}-R^{L})-L^{L}-. In some embodiments, X is —N═C(-L^{L}-R^{L})-L^{L}-. In some embodiments, X is -L^{L}-N=C(-L^{L}-R^{L})—. In some embodiments, X is —N═C(-L^{L}-R^{L})—. In some embodiments, X is L^{L}. In some embodiments, X is a covalent bond.

In some embodiments, Y is a covalent bond. In some embodiments, Y is —O—. In some embodiments, Y is —N(R′)—. In some embodiments, Z is a covalent bond. In some embodiments, Z is —O—. In some embodiments, Z is —N(R′)—. In some embodiments, R′ is R. In some embodiments, R is —H. In some embodiments, R is optionally substituted C₁₋₆ aliphatic. In some embodiments, R is methyl. In some embodiments, R is ethyl. In some embodiments, R is propyl. In some embodiments, R is optionally substituted phenyl. In some embodiments, R is phenyl.

As described herein, various variables in structures in the present disclosure can be or comprise R. Suitable embodiments for R are described extensively in the present disclosure. As appreciated by those skilled in the art, R embodiments described for a variable that can be R may also be applicable to another variable that can be R. Similarly, embodiments described for a component/moiety (e.g., L) for a variable may also be applicable to other variables that can be or comprise the component/moiety.

In some embodiments, R″ is R′. In some embodiments, R″ is —N(R′)₂.

In some embodiments, —X—R^{L} is —SH. In some embodiments, —X—R^{L} is —OH.

In some embodiments, —X—R^{L} is —N(R′)₂. In some embodiments, each R′ is independently optionally substituted C₁₋₆ aliphatic. In some embodiments, each R′ is independently methyl.

In some embodiments, a non-negatively charged internucleotidic linkage has the structure of —OP(═O)(—N═C((N(R′)₂)₂—O—. In some embodiments, a R′ group of one N(R′)₂ is R, a R′ group of the other N(R′)₂ is R, and the two R groups are taken together with their intervening atoms to form an optionally substituted ring, e.g., a 5-membered ring as in n001. In some embodiments, each R′ is independently R, wherein each R is independently optionally substituted C₁₋₆ aliphatic.

In some embodiments, —X—R^{L} is —N=C(-L^{L}-R′)₂. In some embodiments, —X—R^{L} is —N=C(-L^{L1}-L^{L2}-L^{L3}-R′)₂, wherein each L^{L1}, L^{L2} and L^{L3} is independently L″, wherein each L″ is independently a covalent bond, or a bivalent, optionally substituted, linear or branched group selected from a C₁₋₁₀ aliphatic group and a C₁₋₁₀ heteroaliphatic group having 1-5 heteroatoms, wherein one or more methylene units are optionally and independently replaced by an optionally substituted group selected from C₁₋₆ alkylene, C₁₋₆ alkenylene, —C≡C—, a bivalent C₁-C₆ heteroaliphatic group having 1-5 heteroatoms, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, —C(O)O—, —P(O)(OR′)—, —P(O)(SR′)—, —P(O)(R′)—, —P(O)(NR′)—, —P(S)(OR′)—, —P(S)(SR′)—, —P(S)(R′)—, —P(S)(NR′)—, —P(R′)—, —P(OR′)—, —P(SR′)—, —P(NR′)—, —P(OR′)[B(R′)₃]—, —OP(O)(OR′)O—, —OP(O)(SR′)O—, —OP(O)(R′)O—, —OP(O)(NR′)O—, —OP(OR′)O—, —OP(SR′)O—, —OP(NR′)O—, —OP(R′)O—, or —OP(OR′)[B(R′)₃]O—, and one or more nitrogen or carbon atoms are optionally and independently replaced with Cy^{L}. In some embodiments, L^{L2} is -Cy-. In some embodiments, L^{L}1 is a covalent bond. In some embodiments, L^{L3} is a covalent bond. In some embodiments, —X—R^{L} is —N═C(-L^{L1}-Cy-L^{L3}-R′)₂. In some embodiments, —X—R^{L} is

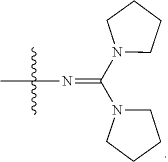 [SMILES: *N=C(N1CCCC1)N1CCCC1]

In some embodiments, —X—R^{L} is

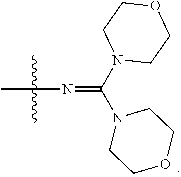 [SMILES: *N=C(N1CCOCC1)N1CCOCC1]

In some embodiments, —X—R^{L} is

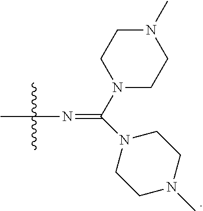 [SMILES: *N=C(N1CCN(C)CC1)N1CCN(C)CC1]

In some embodiments, —X—R^{L} is

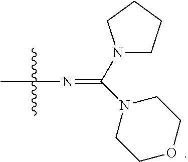 [SMILES: *N=C(N1CCCC1)N1CCOCC1]

In some embodiments, —X—R^{L} is

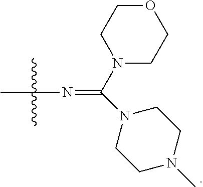 [SMILES: *N=C(N1CCOCC1)N1CCN(C)CC1]

In some embodiments, —X—R^{L} is

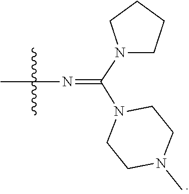 [SMILES: *N=C(N1CCCC1)N1CCN(C)CC1]

In some embodiments, as utilized in the present disclosure, L is covalent bond. In some embodiments, L is a bivalent, optionally substituted, linear or branched group selected from a C₁₋₃₀ aliphatic group and a C₁₋₃₀ heteroaliphatic group having 1-10 heteroatoms, wherein one or more methylene units are optionally and independently replaced by an optionally substituted group selected from C₁₋₆ alkylene, C₁₋₆ alkenylene, —C≡C—, a bivalent C₁-C₆ heteroaliphatic group having 1-5 heteroatoms, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, —C(O)O—, —P(O)(OR′)—, —P(O)(SR′)—, —P(O)(R′)—, —P(O)(NR′)—, —P(S)(OR′)—, —P(S)(SR′)—, —P(S)(R′)—, —P(S)(NR′)—, —P(R′)—, —P(OR′)—, —P(SR′)—, —P(NR′)—, —P(OR′)[B(R′)₃]—, —OP(O)(OR′)O—, —OP(O)(SR′)O—, —OP(O)(R′)O—, —OP(O)(NR′)O—, —OP(OR′)O—, —OP(SR′)O—, —OP(NR′)O—, —OP(R′)O—, or —OP(OR′)[B(R′)₃]O—, and one or more nitrogen or carbon atoms are optionally and independently replaced with Cy^{L}. In some embodiments, L is a bivalent, optionally substituted, linear or branched group selected from a C₁₋₃₀ aliphatic group and a C₁₋₃₀ heteroaliphatic group having 1-10 heteroatoms, wherein one or more methylene units are optionally and independently replaced by an optionally substituted group selected from —C≡C—, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, —C(O)O—, —P(O)(OR′)—, —P(O)(SR′)—, —P(O)(R′)—, —P(O)(NR′)—, —P(S)(OR′)—, —P(S)(SR′)—, —P(S)(R′)—, —P(S)(NR′)—, —P(R′)—, —P(OR′)—, —P(SR′)—, —P(NR′)—, —P(OR′)[B(R′)₃]—, —OP(O)(OR′)O—, —OP(O)(SR′)O—, —OP(O)(R′)O—, —OP(O)(NR′)O—, —OP(OR′)O—, —OP(SR′)O—, —OP(NR′)O—, —OP(R′)O—, or —OP(OR′)[B(R′)₃]O—, and one or more nitrogen or carbon atoms are optionally and independently replaced with Cy^{L}. In some embodiments, L is a bivalent, optionally substituted, linear or branched group selected from a C₁₋₁₀ aliphatic group and a C₁₋₁₀ heteroaliphatic group having 1-10 heteroatoms, wherein one or more methylene units are optionally and independently replaced by an optionally substituted group selected from —C≡C—, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, —C(O)O—, —P(O)(OR′)—, —P(O)(SR′)—, —P(O)(R′)—, —P(O)(NR′)—, —P(S)(OR′)—, —P(S)(SR′)—, —P(S)(R′)—, —P(S)(NR′)—, —P(R′)—, —P(OR′)—, —P(SR′)—, —P(NR′)—, —P(OR′)[B(R′)₃]—, —OP(O)(OR′)O—, —OP(O)(SR′)O—, —OP(O)(R′)O—, —OP(O)(NR′)O—, —OP(OR′)O—, —OP(SR′)O—, —OP(NR′)O—, —OP(R′)O—, or —OP(OR′)[B(R′)₃]O—, and one or more nitrogen or carbon atoms are optionally and independently replaced with Cy^{L}. In some embodiments, one or more methylene units are optionally and independently replaced by an optionally substituted group selected from —C≡C—, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—.

In some embodiments, an internucleotidic linkage is a phosphoryl guanidine internucleotidic linkage. In some embodiments, —X—R^{L} is —N═C[N(R′)₂]2. In some embodiments, each R′ is independently R. In some embodiments, R is optionally substituted C₁₋₆ aliphatic. In some embodiments, R is methyl. In some embodiments, —X—R^{L} is

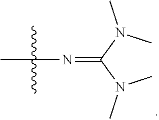 [SMILES: *N=C(N(C)C)N(C)C]

In some embodiments, one R′ on a nitrogen atom is taken with a R′ on the other nitrogen to form a ring as described herein.

In some embodiments, —X—R^{L} is

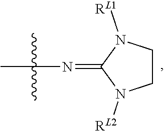

wherein R¹ and R² are independently R′. In some embodiments, —X—R^{L} is

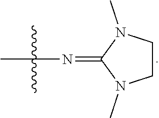 [SMILES: *N=C1N(C)CCN1C]

In some embodiments, —X—R^{L} is

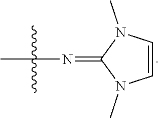 [SMILES: *N=c1n(C)ccn1C]

In some embodiments, two R′ on the same nitrogen are taken together to form a ring as described herein. In some embodiments, —X—R^{L} is

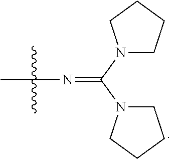 [SMILES: *N=C(N1CCCC1)N1CCCC1]

In some embodiments, —X—R^{L} is

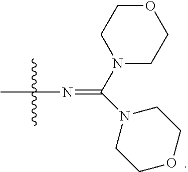 [SMILES: *N=C(N1CCOCC1)N1CCOCC1]

In some embodiments, —X—R^{L} is

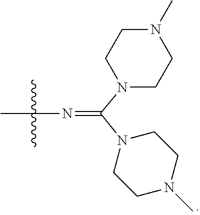 [SMILES: *N=C(N1CCN(C)CC1)N1CCN(C)CC1]

In some embodiments, —X—R^{L} is

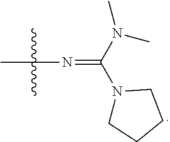 [SMILES: *N=C(N(C)C)N1CCCC1]

In some embodiments, —X—R^{L} is

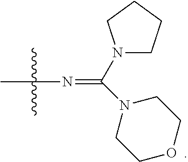 [SMILES: *N=C(N1CCCC1)N1CCOCC1]

In some embodiments, —X—R^{L} is

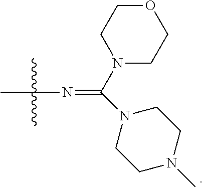 [SMILES: *N=C(N1CCOCC1)N1CCN(C)CC1]

In some embodiments, —X—R^{L} is

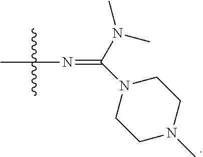 [SMILES: *N=C(N(C)C)N1CCN(C)CC1]

In some embodiments, —X—R^{L} is

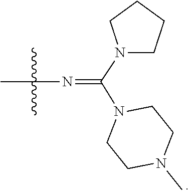 [SMILES: *N=C(N1CCCC1)N1CCN(C)CC1]

In some embodiments, —X—R^{L} is

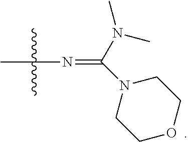 [SMILES: *N=C(N(C)C)N1CCOCC1]

In some embodiments, —X—R^{L} is R as described herein. In some embodiments, R is not hydrogen. In some embodiments, R is optionally substituted C₁₋₆ aliphatic. In some embodiments, R is optionally substituted C₁₋₆ alkyl. In some embodiments, R is methyl.

In some embodiments, —X—R^{L} is selected from Tables below. In some embodiments, X is as described herein. In some embodiments, R^{L} is as described herein. In some embodiments, a linkage has the structure of —Y—P^{L}(—X—R^{L})—Z—, wherein —X—R^{L} is selected from Tables below, and each other variable is independently as described herein. In some embodiments, a linkage has the structure of or comprises —P(O)(—X—R^{L})—, wherein —X—R^{L} is selected from Tables below. In some embodiments, a linkage has the structure of or comprises —P(S)(—X—R^{L})—, wherein —X—R^{L} is selected from Tables below. In some embodiments, a linkage has the structure of or comprises —P(—X—R^{L})—, wherein —X—R^{L} is selected from Tables below. In some embodiments, a linkage has the structure of or comprises —O—P(O)(—X—R^{L})—O—, wherein —X—R^{L} is selected from Tables below. In some embodiments, a linkage has the structure of or comprises —O—P(S)(—X—R^{L})—O—, wherein —X—R^{L} is selected from Tables below. In some embodiments, a linkage has the structure of or comprises —O—P(—X—R^{L})—O—, wherein —X—R^{L} is selected from Tables below. In some embodiments, a linkage has the structure of —O—P(O)(—X—R^{L})—O—, wherein —X—R^{L} is selected from Tables below. In some embodiments, a linkage has the structure of —O—P(S)(—X—R^{L})—O—, wherein —X—R^{L} is selected from Tables below. In some embodiments, a linkage has the structure of —O—P(—X—R^{L})—, wherein —X—R^{L} is selected from Tables below. In some embodiments, the Tables below, n is 0-20 or as described herein.

TABLE L-1
Certain useful moieties bonded to linkage phosphorus (e.g., —X—R^{L}).

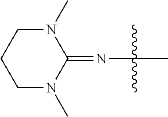

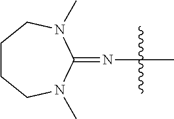

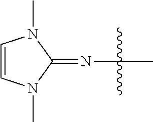

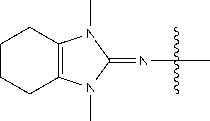

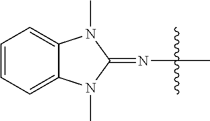

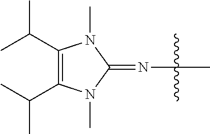

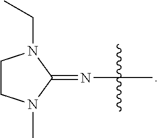

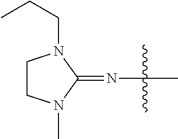

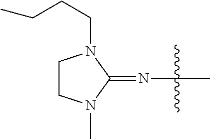

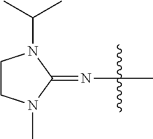

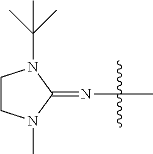

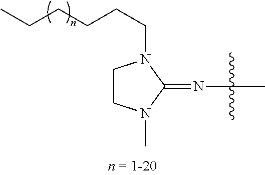

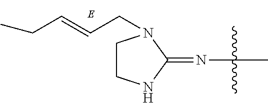

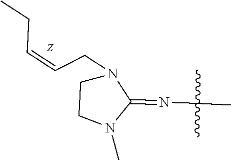

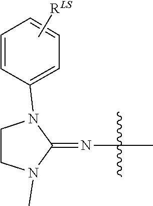

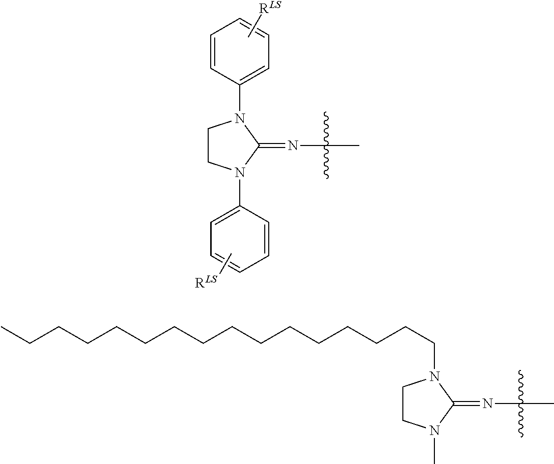

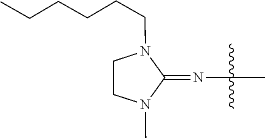

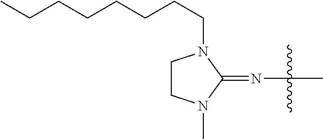

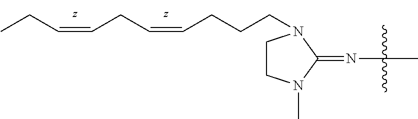

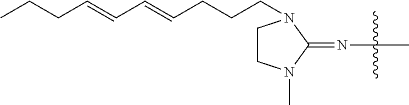

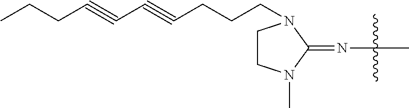

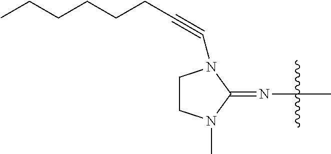

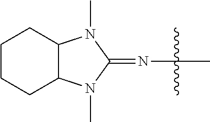

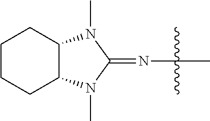

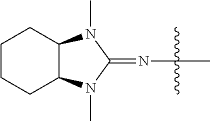

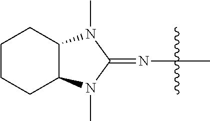

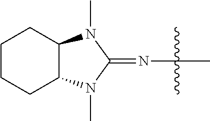

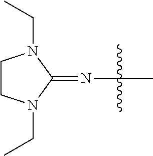

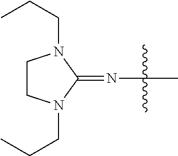

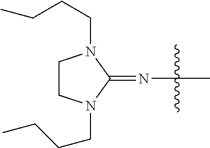

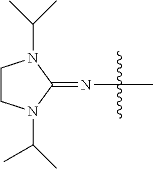

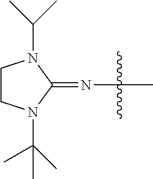

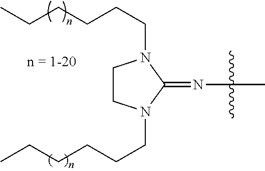

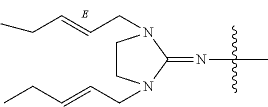

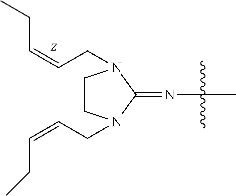

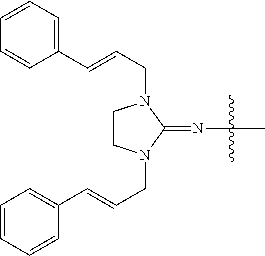

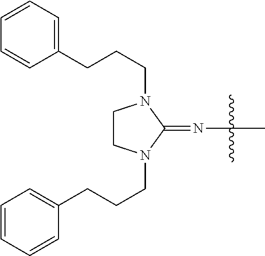

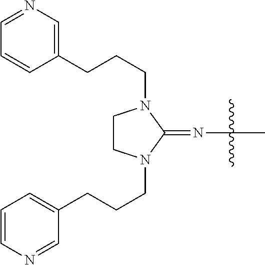

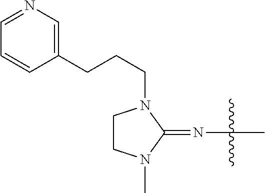

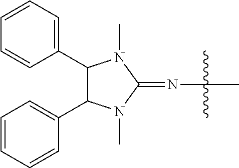

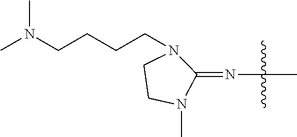

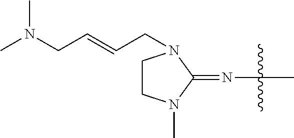

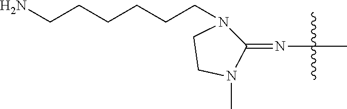

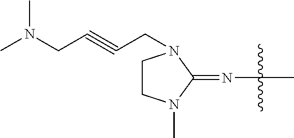

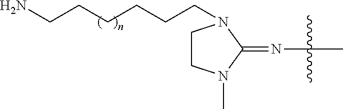

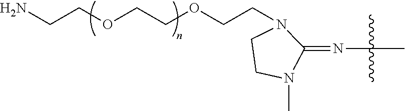

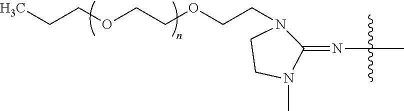

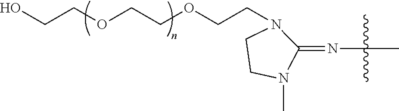

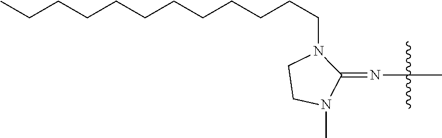

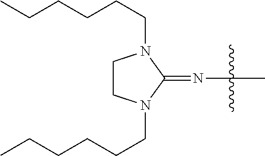

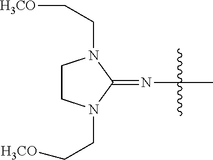

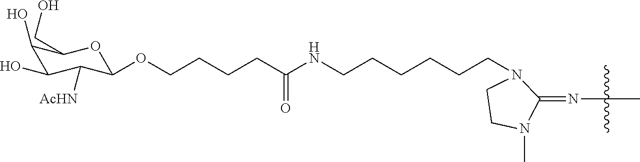

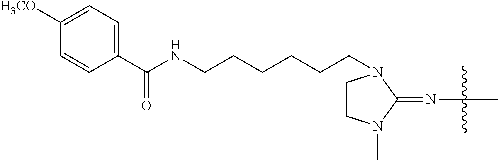

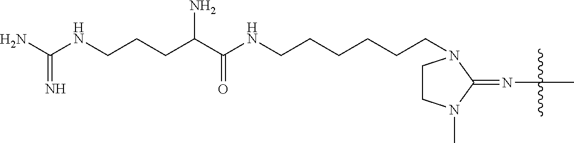

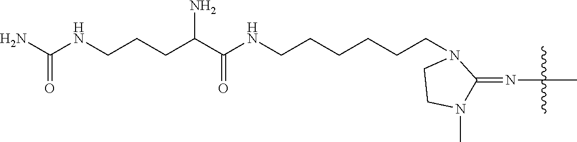

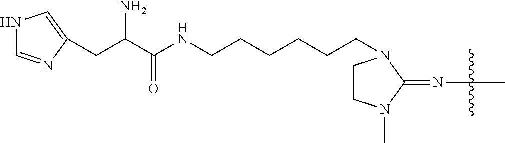

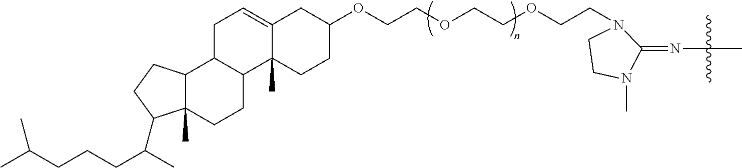

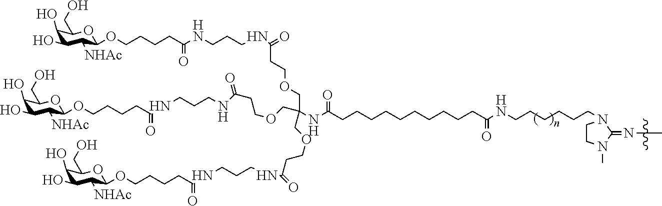

wherein each R^{LS} is independently R^{S}. In some embodiments, each R^{LS} is independently
—Cl, —Br, —F, —N(Me)₂, or —NHCOCH₃.

TABLE L-2
Certain useful moieties bonded to linkage phosphorus
(e.g., —X—R^{L}).

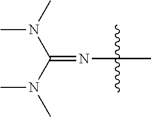 [SMILES: *N=C(N(C)C)N(C)C]

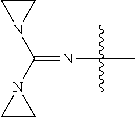 [SMILES: *N=C(N1CC1)N1CC1]

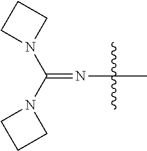 [SMILES: *N=C(N1CCC1)N1CCC1]

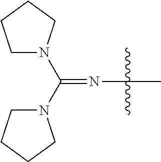 [SMILES: *N=C(N1CCCC1)N1CCCC1]

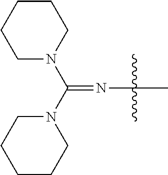 [SMILES: *N=C(N1CCCCC1)N1CCCCC1]

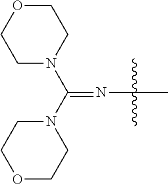 [SMILES: *N=C(N1CCOCC1)N1CCOCC1]

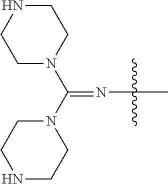 [SMILES: *N=C(N1CCNCC1)N1CCNCC1]

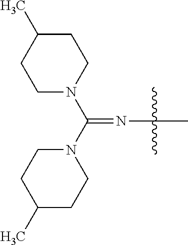 [SMILES: *N=C(N1CCC(C)CC1)N1CCC(C)CC1]

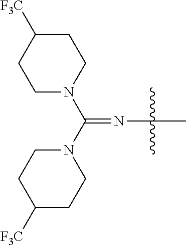 [SMILES: *N=C(N1CCC(C(F)(F)F)CC1)N1CCC(C(F)(F)F)CC1]

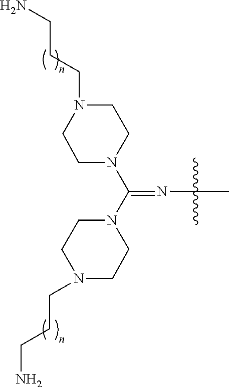

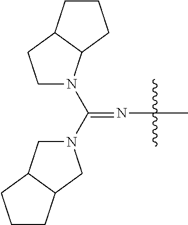 [SMILES: *N=C(N1CC2CCCC2C1)N1CCC2CCCC21]

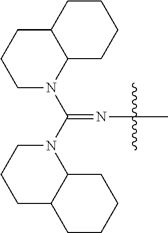 [SMILES: *N=C(N1CCCC2CCCCC21)N1CCCC2CCCCC21]

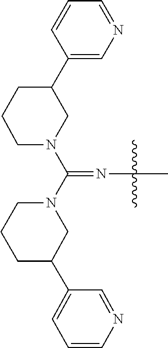 [SMILES: *N=C(N1CCCC(c2cccnc2)C1)N1CCCC(c2cccnc2)C1]

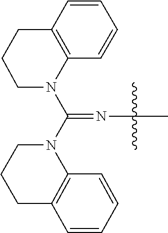

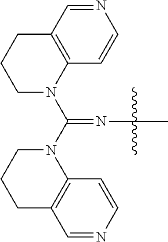 [SMILES: *N=C(N1CCCc2cnccc21)N1CCCc2cnccc21]

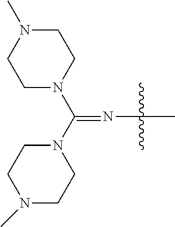 [SMILES: *N=C(N1CCN(C)CC1)N1CCN(C)CC1]

TABLE L-3
Certain useful moieties bonded to linkage phosphorus
(e.g., —X—R^{L}).

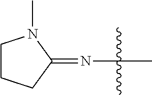 [SMILES: *N=C1CCCN1C]

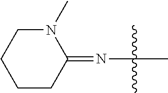 [SMILES: *N=C1CCCCN1C]

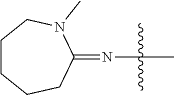 [SMILES: *N=C1CCCCCN1C]

TABLE L-4
Certain useful moieties bonded to linkage phosphorus
(e.g., —X—R^{L}).

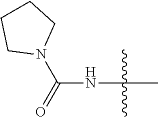 [SMILES: *NC(=O)N1CCCC1]

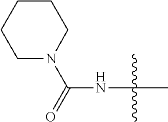 [SMILES: *NC(=O)N1CCCCC1]

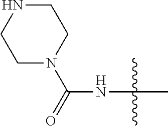 [SMILES: *NC(=O)N1CCNCC1]

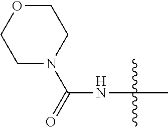 [SMILES: *NC(=O)N1CCOCC1]

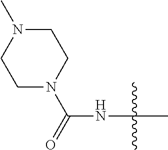 [SMILES: *NC(=O)N1CCN(C)CC1]

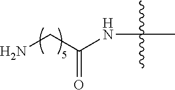

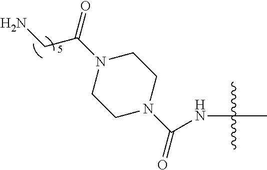

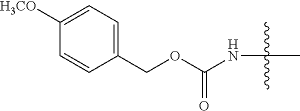 [SMILES: *NC(=O)OCc1ccc(OC)cc1]

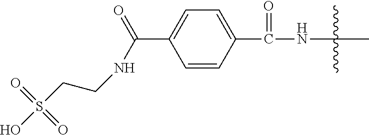 [SMILES: *NC(=O)c1ccc(C(=O)NCCS(=O)(=O)O)cc1]

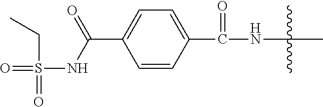 [SMILES: *NC(=O)c1ccc(C(=O)NS(=O)(=O)CC)cc1]

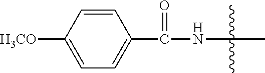 [SMILES: *NC(=O)c1ccc(OC)cc1]

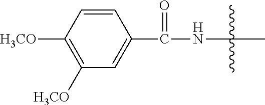 [SMILES: *NC(=O)c1ccc(OC)c(OC)c1]

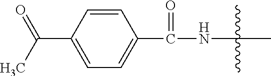 [SMILES: *NC(=O)c1ccc(C(C)=O)cc1]

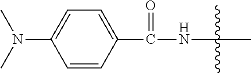 [SMILES: *NC(=O)c1ccc(N(C)C)cc1]

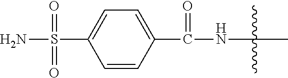 [SMILES: *NC(=O)c1ccc(S(N)(=O)=O)cc1]

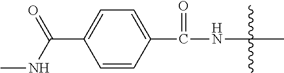 [SMILES: *NC(=O)c1ccc(C(=O)NC)cc1]

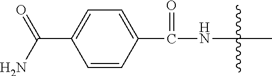 [SMILES: *NC(=O)c1ccc(C(N)=O)cc1]

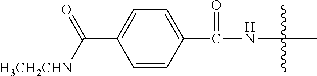

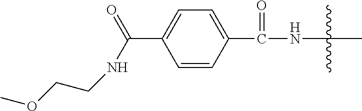 [SMILES: *NC(=O)c1ccc(C(=O)NCCOC)cc1]

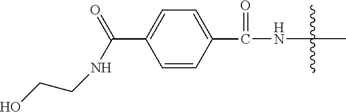 [SMILES: *NC(=O)c1ccc(C(=O)NCCO)cc1]

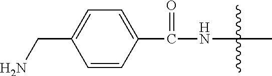 [SMILES: *NC(=O)c1ccc(CN)cc1]

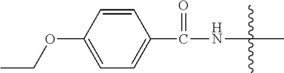 [SMILES: *NC(=O)c1ccc(OCC)cc1]

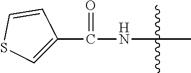 [SMILES: *NC(=O)c1ccsc1]

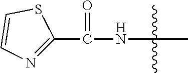 [SMILES: *NC(=O)c1nccs1]

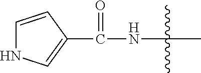 [SMILES: *NC(=O)c1cc[nH]c1]

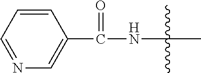 [SMILES: *NC(=O)c1cccnc1]

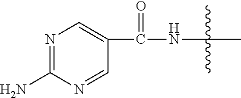 [SMILES: *NC(=O)c1cnc(N)nc1]

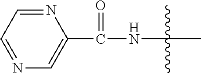 [SMILES: *NC(=O)c1cnccn1]

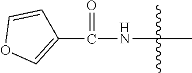 [SMILES: *NC(=O)c1ccoc1]

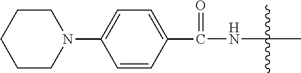 [SMILES: *NC(=O)c1ccc(N2CCCCC2)cc1]

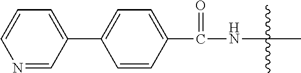 [SMILES: *NC(=O)c1ccc(-c2cccnc2)cc1]

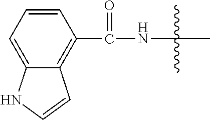 [SMILES: *NC(=O)c1cccc2[nH]ccc12]

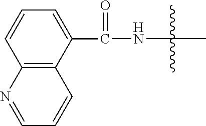 [SMILES: *NC(=O)c1cccc2ncccc12]

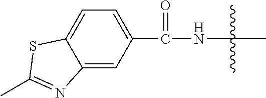 [SMILES: *NC(=O)c1ccc2sc(C)nc2c1]

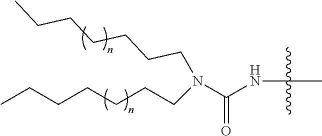

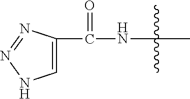 [SMILES: *NC(=O)c1c[nH]nn1]

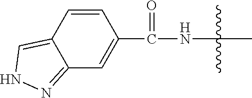 [SMILES: *NC(=O)c1ccc2c[nH]nc2c1]

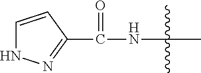 [SMILES: *NC(=O)c1cc[nH]n1]

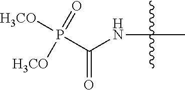 [SMILES: *NC(=O)P(=O)(OC)OC]

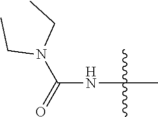 [SMILES: *NC(=O)N(CC)CC]

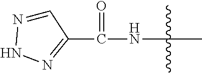 [SMILES: *NC(=O)c1cn[nH]n1]

TABLE L-5
Certain useful moieties bonded to linkage phosphorus (e.g., —X—R^{L}).

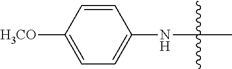 [SMILES: *Nc1ccc(OC)cc1]

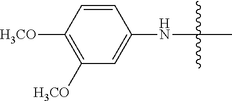 [SMILES: *Nc1ccc(OC)c(OC)c1]

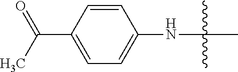 [SMILES: *Nc1ccc(C(C)=O)cc1]

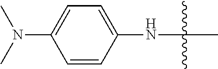 [SMILES: *Nc1ccc(N(C)C)cc1]

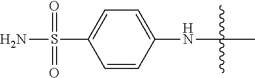 [SMILES: *Nc1ccc(S(N)(=O)=O)cc1]

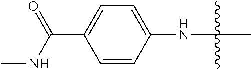 [SMILES: *Nc1ccc(C(=O)NC)cc1]

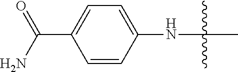 [SMILES: *Nc1ccc(C(N)=O)cc1]

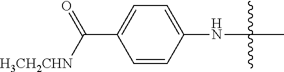

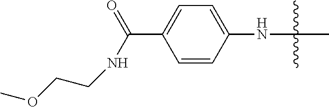 [SMILES: *Nc1ccc(C(=O)NCCOC)cc1]

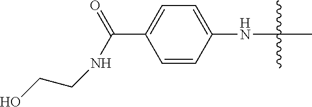 [SMILES: *Nc1ccc(C(=O)NCCO)cc1]

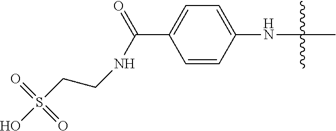 [SMILES: *Nc1ccc(C(=O)NCCS(=O)(=O)O)cc1]

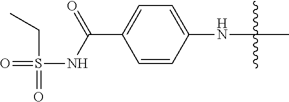 [SMILES: *Nc1ccc(C(=O)NS(=O)(=O)CC)cc1]

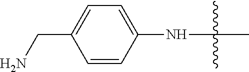 [SMILES: *Nc1ccc(CN)cc1]

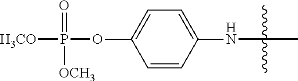 [SMILES: *Nc1ccc(OP(=O)(OC)OC)cc1]

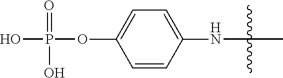 [SMILES: *Nc1ccc(OP(=O)(O)O)cc1]

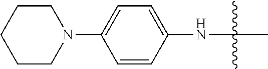 [SMILES: *Nc1ccc(N2CCCCC2)cc1]

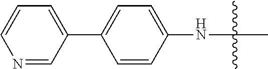 [SMILES: *Nc1ccc(-c2cccnc2)cc1]

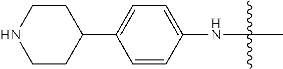 [SMILES: *Nc1ccc(C2CCNCC2)cc1]

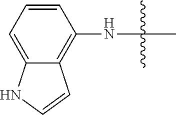 [SMILES: *Nc1cccc2[nH]ccc12]

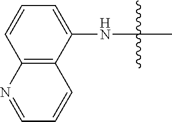 [SMILES: *Nc1cccc2ncccc12]

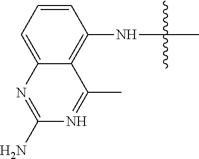 [SMILES: *Nc1cccc2nc(N)nc(C)c12]

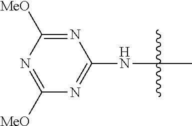 [SMILES: *Nc1nc(OC)nc(OC)n1]

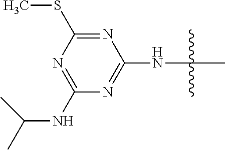 [SMILES: *Nc1nc(NC(C)C)nc(SC)n1]

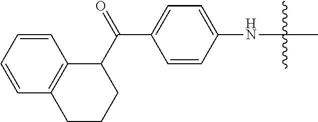 [SMILES: *Nc1ccc(C(=O)C2CCCc3ccccc32)cc1]

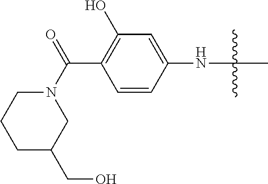 [SMILES: *Nc1ccc(C(=O)N2CCCC(CO)C2)c(O)c1]

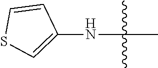 [SMILES: *Nc1ccsc1]

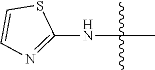 [SMILES: *Nc1nccs1]

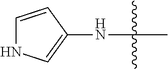 [SMILES: *Nc1cc[nH]c1]

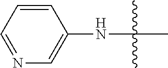 [SMILES: *Nc1cccnc1]

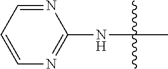 [SMILES: *Nc1ncccn1]

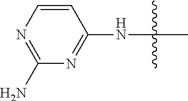 [SMILES: *Nc1ccnc(N)n1]

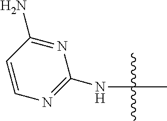 [SMILES: *Nc1nccc(N)n1]

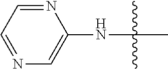 [SMILES: *Nc1cnccn1]

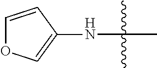 [SMILES: *Nc1ccoc1]

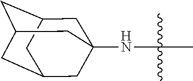 [SMILES: *NC12CC3CC(CC(C3)C1)C2]

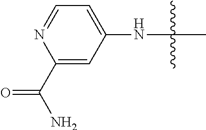 [SMILES: *Nc1ccnc(C(N)=O)c1]

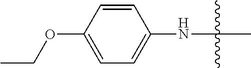 [SMILES: *Nc1ccc(OCC)cc1]

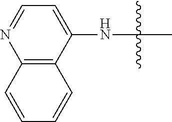 [SMILES: *Nc1ccnc2ccccc12]

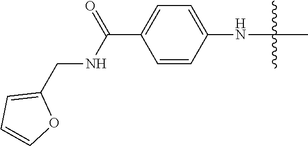 [SMILES: *Nc1ccc(C(=O)NCc2ccco2)cc1]

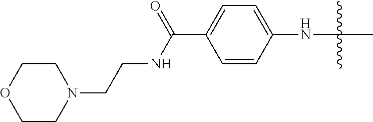 [SMILES: *Nc1ccc(C(=O)NCCN2CCOCC2)cc1]

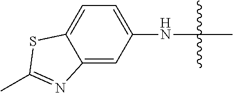 [SMILES: *Nc1ccc2sc(C)nc2c1]

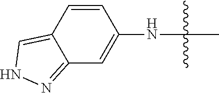 [SMILES: *Nc1ccc2c[nH]nc2c1]

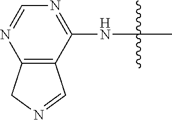 [SMILES: *Nc1ncnc2c1C=NC2]

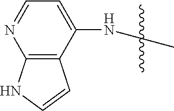 [SMILES: *Nc1ccnc2[nH]ccc12]

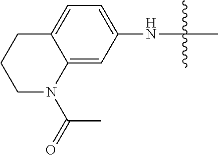 [SMILES: *Nc1ccc2c(c1)N(C(C)=O)CCC2]

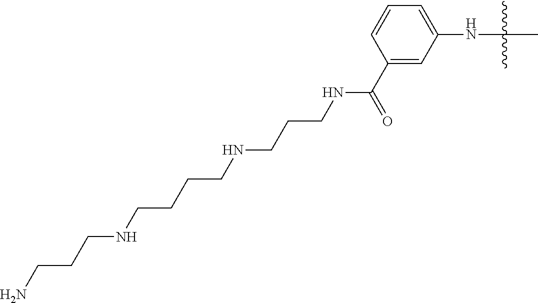 [SMILES: *Nc1cccc(C(=O)NCCCNCCCCNCCCN)c1]

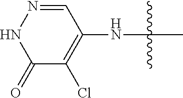 [SMILES: *Nc1cn[nH]c(=O)c1Cl]

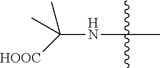 [SMILES: *NC(C)(C)C(=O)O]

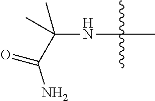 [SMILES: *NC(C)(C)C(N)=O]

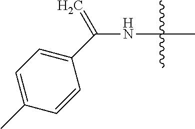 [SMILES: *NC(=C)c1ccc(C)cc1]

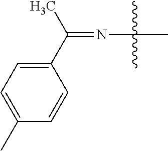 [SMILES: *N=C(C)c1ccc(C)cc1]

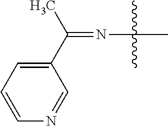 [SMILES: *N=C(C)c1cccnc1]

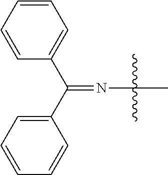 [SMILES: *N=C(c1ccccc1)c1ccccc1]

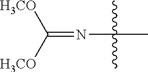 [SMILES: *N=C(OC)OC]

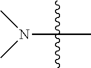 [SMILES: *N(C)C]

TABLE L-6
Certain useful moieties bonded to linkage phosphorus
(e.g., —X—R^{L}).

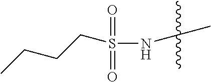 [SMILES: *NS(=O)(=O)CCCC]

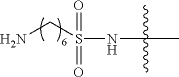

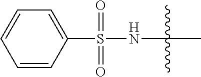 [SMILES: *NS(=O)(=O)c1ccccc1]

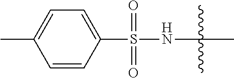 [SMILES: *NS(=O)(=O)c1ccc(C)cc1]

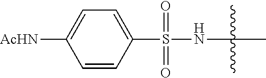 [SMILES: *NS(=O)(=O)c1ccc(NC(C)=O)cc1]

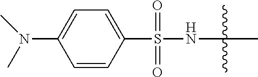 [SMILES: *NS(=O)(=O)c1ccc(N(C)C)cc1]

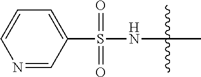 [SMILES: *NS(=O)(=O)c1cccnc1]

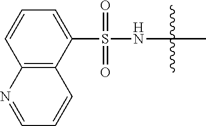 [SMILES: *NS(=O)(=O)c1cccc2ncccc12]

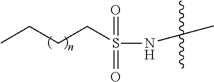

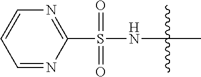 [SMILES: *NS(=O)(=O)c1ncccn1]

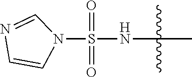 [SMILES: *NS(=O)(=O)n1ccnc1]

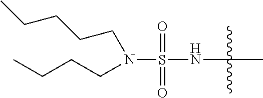 [SMILES: *NS(=O)(=O)N(CCCC)CCCCC]

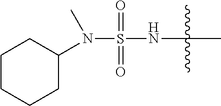 [SMILES: *NS(=O)(=O)N(C)C1CCCCC1]

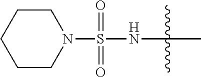 [SMILES: *NS(=O)(=O)N1CCCCC1]

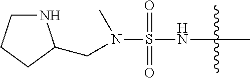 [SMILES: *NS(=O)(=O)N(C)CC1CCCN1]

In some embodiments, an internucleotidic linkage, e.g., a non-negatively charged internucleotidic linkage or a neutral internucleotidic linkage, has the structure of -L^{L1}-Cy^{IL}-L^{L2}-. In some embodiments, L^{L1} is bonded to a 3′-carbon of a sugar. In some embodiments, L^{L2} is bonded to a 5′-carbon of a sugar. In some embodiments, L^{L1} is —O—CH₂—. In some embodiments, L^{L2} is a covalent bond. In some embodiments, L^{L2} is a —N(R′)—. In some embodiments, L^{L2} is a —NH—. In some embodiments, L^{L2} is bonded to a 5′-carbon of a sugar, which 5′-carbon is substituted with ═O. In some embodiments, Cy^{IL} is optionally substituted 3-10 membered saturated, partially unsaturated, or aromatic ring having 0-5 heteroatoms. In some embodiments, Cy^{IL} is an optionally substituted triazole ring. In some embodiments, Cy^{IL} is

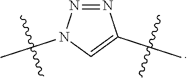 [SMILES: *c1cn(*)nn1]

In some embodiments, a linkage is

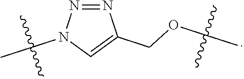 [SMILES: *OCc1cn(*)nn1]

In some embodiments, a non-negatively charged internucleotidic linkage has the structure of —OP(═W)(—N(R′)₂)—O—.

In some embodiments, R′ is R. In some embodiments, R′ is H. In some embodiments, R′ is —C(O)R. In some embodiments, R′ is —C(O)OR. In some embodiments, R′ is —S(O)₂R.

In some embodiments, R″ is —NHR′. In some embodiments, —N(R′)₂ is —NHR′.

As described herein, some embodiments, R is H. In some embodiments, R is optionally substituted C₁₋₆ aliphatic. In some embodiments, R is optionally substituted C₁₋₆ alkyl. In some embodiments, R is methyl. In some embodiments, R is substituted methyl. In some embodiments, R is ethyl. In some embodiments, R is substituted ethyl.

In some embodiments, as described herein, a non-negatively charged internucleotidic linkage is a neutral internucleotidic linkage.

In some embodiments, a modified internucleotidic linkage (e.g., a non-negatively charged internucleotidic linkage) comprises optionally substituted triazolyl. In some embodiments, R′ is or comprises optionally substituted triazolyl. In some embodiments, a modified internucleotidic linkage (e.g., a non-negatively charged internucleotidic linkage) comprises optionally substituted alkynyl. In some embodiments, R′ is optionally substituted alkynyl. In some embodiments, R′ comprises an optionally substituted triple bond. In some embodiments, a modified internucleotidic linkage comprises a triazole or alkyne moiety. In some embodiments, R′ is or comprises an optionally substituted triazole or alkyne moiety. In some embodiments, a triazole moiety, e.g., a triazolyl group, is optionally substituted. In some embodiments, a triazole moiety, e.g., a triazolyl group) is substituted. In some embodiments, a triazole moiety is unsubstituted. In some embodiments, a modified internucleotidic linkage comprises an optionally substituted guanidine moiety. In some embodiments, a modified internucleotidic linkage comprises an optionally substituted cyclic guanidine moiety. In some embodiments, R′, R^{L}, or —X—R^{L}, is or comprises an optionally substituted guanidine moiety. In some embodiments, R′, R^{L}, or —X—R^{L}, is or comprises an optionally substituted cyclic guanidine moiety. In some embodiments, R′, R^{L}, or —X—R^{L} comprises an optionally substituted cyclic guanidine moiety and an internucleotidic linkage has the structure of:

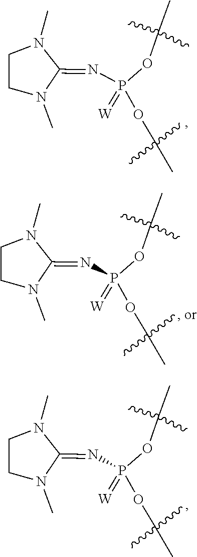

wherein W is O or S. In some embodiments, W is O. In some embodiments, W is S. In some embodiments, a non-negatively charged internucleotidic linkage is stereochemically controlled.

In some embodiments, a non-negatively charged internucleotidic linkage or a neutral internucleotidic linkage is an internucleotidic linkage comprising a triazole moiety. In some embodiments, a non-negatively charged internucleotidic linkage or a non-negatively charged interucleotidic linkage comprises an optionally substituted triazolyl group. In some embodiments, an internucleotidic linkage comprising a triazole moiety (e.g., an optionally substituted triazolyl group) has the structure of

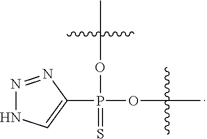 [SMILES: *OP(=S)(O*)c1c[nH]nn1]

In some embodiments, an internucleotidic linkage comprising a triazole moiety has the structure of

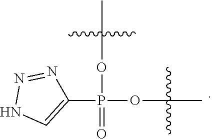 [SMILES: *OP(=O)(O*)c1c[nH]nn1]

In some embodiments, an internucleotidic linkage, e.g., a non-negatively charged internucleotidic linkage, a neutral internucleotidic linkage, comprises a cyclic guanidine moiety. In some embodiments, an interucleotidic linkage comprising a cyclic guanidine moiety has the structure of

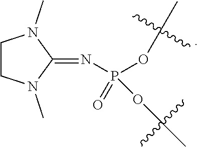 [SMILES: *OP(=O)(N=C1N(C)CCN1C)O*]

In some embodiments, a non-negatively charged interucleotidic linkage, or a neutral internucleotidic linkage, is or comprising a structure selected from

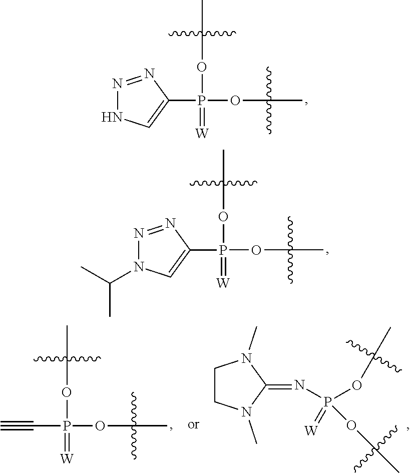

wherein W is O or S.

In some embodiments, an internucleotidic linkage comprises a Tmg group

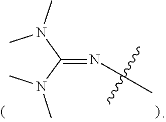 [SMILES: *N=C(N(C)C)N(C)C]

In some embodiments, an internucleotidic linkage comprises a Tmg group and has the structure of

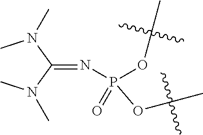 [SMILES: *OP(=O)(N=C(N(C)C)N(C)C)O*]

(the “Tmg internucleotidic linkage”). In some embodiments, neutral internucleotidic linkages include internucleotidic linkages of PNA and PMO, and an Tmg internucleotidic linkage.

In some embodiments, a non-negatively charged internucleotidic linkage comprises an optionally substituted 3-20 membered heterocyclyl or heteroaryl group having 1-10 heteroatoms. In some embodiments, a non-negatively charged internucleotidic linkage comprises an optionally substituted 3-20 membered heterocyclyl or heteroaryl group having 1-10 heteroatoms, wherein at least one heteroatom is nitrogen. In some embodiments, such a heterocyclyl or heteroaryl group is of a 5-membered ring. In some embodiments, such a heterocyclyl or heteroaryl group is of a 6-membered ring.

In some embodiments, a non-negatively charged internucleotidic linkage comprises an optionally substituted 5-20 membered heteroaryl group having 1-10 heteroatoms. In some embodiments, a non-negatively charged internucleotidic linkage comprises an optionally substituted 5-20 membered heteroaryl group having 1-10 heteroatoms, wherein at least one heteroatom is nitrogen. In some embodiments, a non-negatively charged internucleotidic linkage comprises an optionally substituted 5-6 membered heteroaryl group having 1-4 heteroatoms, wherein at least one heteroatom is nitrogen. In some embodiments, a non-negatively charged internucleotidic linkage comprises an optionally substituted 5-membered heteroaryl group having 1-4 heteroatoms, wherein at least one heteroatom is nitrogen. In some embodiments, a heteroaryl group is directly bonded to a linkage phosphorus. In some embodiments, a non-negatively charged internucleotidic linkage comprises an optionally substituted 5-20 membered heterocyclyl group having 1-10 heteroatoms. In some embodiments, a non-negatively charged internucleotidic linkage comprises an optionally substituted 5-20 membered heterocyclyl group having 1-10 heteroatoms, wherein at least one heteroatom is nitrogen. In some embodiments, a non-negatively charged internucleotidic linkage comprises an optionally substituted 5-6 membered heterocyclyl group having 1-4 heteroatoms, wherein at least one heteroatom is nitrogen. In some embodiments, a non-negatively charged internucleotidic linkage comprises an optionally substituted 5-membered heterocyclyl group having 1-4 heteroatoms, wherein at least one heteroatom is nitrogen. In some embodiments, at least two heteroatoms are nitrogen. In some embodiments, a heterocyclyl group is directly bonded to a linkage phosphorus. In some embodiments, a heterocyclyl group is bonded to a linkage phosphorus through a linker, e.g., ═N— when the heterocyclyl group is part of a guanidine moiety who directed bonded to a linkage phosphorus through its ═N—. In some embodiments, a non-negatively charged internucleotidic linkage comprises an optionally substituted

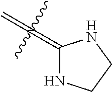 [SMILES: *=C1NCCN1]

group. In some embodiments, anon-negatively charged internucleotidic linkage comprises a substituted

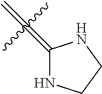 [SMILES: *=C1NCCN1]

group. In some embodiments, a non-negatively charged internucleotidic linkage comprises a

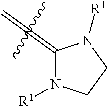

group. In some embodiments, each R¹ is independently optionally substituted C₁₋₆ alkyl. In some embodiments, each R¹ is independently methyl.

In some embodiments, a non-negatively charged internucleotidic linkage, e.g., a neutral internucleotidic linkage is not chirally controlled. In some embodiments, a non-negatively charged internucleotidic linkage is chirally controlled. In some embodiments, a non-negatively charged internucleotidic linkage is chirally controlled and its linkage phosphorus is Rp. In some embodiments, a non-negatively charged internucleotidic linkage is chirally controlled and its linkage phosphorus is Sp.

In some embodiments, an internucleotidic linkage comprises no linkage phosphorus. In some embodiments, an internucleotidic linkage has the structure of —C(O)—(O)— or —C(O)—N(R′)—, wherein R′ is as described herein. In some embodiments, an internucleotidic linkage has the structure of —C(O)—(O)—. In some embodiments, an internucleotidic linkage has the structure of —C(O)—N(R′)—, wherein R′ is as described herein. In various embodiments, —C(O)— is bonded to nitrogen. In some embodiments, an internucleotidic linkage is or comprises —C(O)—O— which is part of a carbamate moiety. In some embodiments, an internucleotidic linkage is or comprises —C(O)—O— which is part of a urea moiety.

In some embodiments, an oligonucleotide comprises 1-20, 1-15, 1-10, 1-5, or 1, 2, 3, 4, 5, 6, 7, 8, 9, 10, or more non-negatively charged internucleotidic linkages. In some embodiments, an oligonucleotide comprises 1-20, 1-15, 1-10, 1-5, or 1, 2, 3, 4, 5, 6, 7, 8, 9, 10, or more neutral internucleotidic linkages. In some embodiments, each of non-negatively charged internucleotidic linkage and/or neutral internucleotidic linkages is optionally and independently chirally controlled. In some embodiments, each non-negatively charged internucleotidic linkage in an oligonucleotide is independently a chirally controlled internucleotidic linkage. In some embodiments, each neutral internucleotidic linkage in an oligonucleotide is independently a chirally controlled internucleotidic linkage. In some embodiments, at least one non-negatively charged internucleotidic linkage/neutral internucleotidic linkage has the structure of

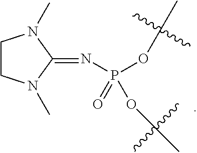 [SMILES: *OP(=O)(N=C1N(C)CCN1C)O*]

In some embodiments, an oligonucleotide comprises at least one non-negatively charged internucleotidic linkage wherein its linkage phosphorus is in Rp configuration, and at least one non-negatively charged internucleotidic linkage wherein its linkage phosphorus is in Sp configuration.

In many embodiments, as demonstrated extensively, oligonucleotides of the present disclosure comprise two or more different internucleotidic linkages. In some embodiments, an oligonucleotide comprises a phosphorothioate internucleotidic linkage and a non-negatively charged internucleotidic linkage. In some embodiments, an oligonucleotide comprises a phosphorothioate internucleotidic linkage, a non-negatively charged internucleotidic linkage, and a natural phosphate linkage. In some embodiments, a non-negatively charged internucleotidic linkage is a neutral internucleotidic linkage. In some embodiments, a non-negatively charged internucleotidic linkage is n001, n003, n004, n006, n008 or n009, n013, n020, n021, n025, n026, n029, n031, n033, n037, n046, n047, n048, n054, or n055). In some embodiments, a non-negatively charged internucleotidic linkage is n001. In some embodiments, each phosphorothioate internucleotidic linkage is independently chirally controlled. In some embodiments, each chiral modified internucleotidic linkage is independently chirally controlled. In some embodiments, one or more non-negatively charged internucleotidic linkage are not chirally controlled.

A typical connection, as in natural DNA and RNA, is that an internucleotidic linkage forms bonds with two sugars (which can be either unmodified or modified as described herein). In many embodiments, as exemplified herein an internucleotidic linkage forms bonds through its oxygen atoms or heteroatoms with one optionally modified ribose or deoxyribose at its 5′ carbon, and the other optionally modified ribose or deoxyribose at its 3′ carbon. In some embodiments, internucleotidic linkages connect sugars that are not ribose sugars, e.g., sugars comprising N ring atoms and acyclic sugars as described herein.

In some embodiments, each nucleoside units connected by an internucleotidic linkage independently comprises a nucleobase which is independently an optionally substituted A, T, C, G, or U, or an optionally substituted tautomer of A, T, C, G or U.

In some embodiments, an oligonucleotide comprises a modified internucleotidic linkage (e.g., a modified internucleotidic linkage having the structure of Formula I, I-a, I-b, or I-c, I-n-1, I-n-2, I-n-3, I-n-4, II, II-a-1, II-a-2, II-b-1, II-b-2, II-c-1, II-c-2, II-d-1, II-d-2, etc., or a salt form thereof) as described in U.S. Pat. Nos. 9,394,333, 9,744,183, 9,605,019, 9,598,458, 9,982,257, U.S. Ser. No. 10/160,969, U.S. Ser. No. 10/479,995, US 2020/0056173, US 2018/0216107, US 2019/0127733, U.S. Ser. No. 10/450,568, US 2019/0077817, US 2019/0249173, US 2019/0375774, WO 2018/223056, WO 2018/223073, WO 2018/223081, WO 2018/237194, WO 2019/032607, WO 2019/055951, WO 2019/075357, WO 2019/200185, WO 2019/217784, and/or WO 2019/032612 the internucleotidic linkages (e.g., those of Formula I, I-a, I-b, or I-c, I-n-1, I-n-2, I-n-3, I-n-4, II, II-a-1, II-a-2, II-b-1, II-b-2, II-c-1, II-c-2, II-d-1, II-d-2, etc.,) of each of which are independently incorporated herein by reference. In some embodiments, a modified internucleotidic linkage is a non-negatively charged internucleotidic linkage. In some embodiments, provided oligonucleotides comprise one or more non-negatively charged internucleotidic linkages. In some embodiments, a non-negatively charged internucleotidic linkage is a positively charged internucleotidic linkage. In some embodiments, a non-negatively charged internucleotidic linkage is a neutral internucleotidic linkage. In some embodiments, the present disclosure provides oligonucleotides comprising one or more neutral internucleotidic linkages. In some embodiments, a non-negatively charged internucleotidic linkage or a neutral internucleotidic linkage (e.g., one of Formula I-n-1, I-n-2, I-n-3, I-n-4, II, II-a-1, II-a-2, II-b-1, II-b-2, II-c-1, II-c-2, II-d-1, II-d-2, etc.) is as described in U.S. Pat. Nos. 9,394,333, 9,744,183, 9,605,019, 9,598,458, 9,982,257, U.S. Ser. No. 10/160,969, U.S. Ser. No. 10/479,995, US 2020/0056173, US 2018/0216107, US 2019/0127733, U.S. Ser. No. 10/450,568, US 2019/0077817, US 2019/0249173, US 2019/0375774, WO 2018/223056, WO 2018/223073, WO 2018/223081, WO 2018/237194, WO 2019/032607, WO 2019/055951, WO 2019/075357, WO 2019/200185, WO 2019/217784, and/or WO 2019/032612. In some embodiments, a non-negatively charged internucleotidic linkage or neutral internucleotidic linkage is one of Formula I-n-1, I-n-2, I-n-3, I-n-4, II, II-a-1, II-a-2, II-b-1, II-b-2, II-c-1, II-c-2, II-d-1, II-d-2, etc. as described in WO 2018/223056, WO 2019/032607, WO 2019/075357, WO 2019/032607, WO 2019/075357, WO 2019/200185, WO 2019/217784, and/or WO 2019/032612, such internucleotidic linkages of each of which are independently incorporated herein by reference.

As described herein, various variables can be R, e.g., R′, R^{L}, etc. Various embodiments for R are described in the present disclosure (e.g., when describing variables that can be R). Such embodiments are generally useful for all variables that can be R. In some embodiments, R is hydrogen. In some embodiments, R is optionally substituted C₁₋₃₀ (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30) aliphatic. In some embodiments, R is optionally substituted C₁₋₂₀ aliphatic. In some embodiments, R is optionally substituted C₁₋₁₀ aliphatic. In some embodiments, R is optionally substituted C₁₋₆ aliphatic. In some embodiments, R is optionally substituted alkyl. In some embodiments, R is optionally substituted C₁₋₆ alkyl. In some embodiments, R is optionally substituted methyl. In some embodiments, R is methyl. In some embodiments, R is optionally substituted ethyl. In some embodiments, R is optionally substituted propyl. In some embodiments, R is isopropyl. In some embodiments, R is optionally substituted butyl. In some embodiments, R is optionally substituted pentyl. In some embodiments, R is optionally substituted hexyl.

In some embodiments, R is optionally substituted 3-30 membered (e.g., 3, 4, 5, 6, 7, 8, 9, 10, 11, 12, 13, 14, 15, 16, 17, 18, 19, 20, 21, 22, 23, 24, 25, 26, 27, 28, 29 or 30) cycloaliphatic. In some embodiments, R is optionally substituted cycloalkyl. In some embodiments, cycloaliphatic is monocyclic, bicyclic, or polycyclic, wherein each monocyclic unit is independently saturated or partially saturated. In some embodiments, R is optionally substituted cyclopropyl. In some embodiments, R is optionally substituted cyclobutyl. In some embodiments, R is optionally substituted cyclopentyl. In some embodiments, R is optionally substituted cyclohexyl. In some embodiments, R is optionally substituted adamantyl.

In some embodiments, R is optionally substituted C₁₋₃₀ (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30) heteroaliphatic having 1-10 heteroatoms. In some embodiments, R is optionally substituted C₁₋₂₀ aliphatic having 1-10 heteroatoms. In some embodiments, R is optionally substituted C₁₋₁₀ aliphatic having 1-10 heteroatoms. In some embodiments, R is optionally substituted C₁₋₆ aliphatic having 1-3 heteroatoms. In some embodiments, R is optionally substituted heteroalkyl. In some embodiments, R is optionally substituted C₁₋₆ heteroalkyl. In some embodiments, R is optionally substituted 3-30 membered (e.g., 3, 4, 5, 6, 7, 8, 9, 10, 11, 12, 13, 14, 15, 16, 17, 18, 19, 20, 21, 22, 23, 24, 25, 26, 27, 28, 29 or 30) heterocycloaliphatic having 1-10 heteroatoms. In some embodiments, R is optionally substituted heterocycloalkyl. In some embodiments, heterocycloaliphatic is monocyclic, bicyclic, or polycyclic, wherein each monocyclic unit is independently saturated or partially saturated.

In some embodiments, R is optionally substituted C₆₋₃₀ aryl. In some embodiments, R is optionally substituted phenyl. In some embodiments, R is optionally substituted phenyl. In some embodiments, R is C₆₋₁₄ aryl. In some embodiments, R is optionally substituted bicyclic aryl. In some embodiments, R is optionally substituted polycyclic aryl. In some embodiments, R is optionally substituted C₆-30 arylaliphatic. In some embodiments, R is C₆₋₃₀ arylheteroaliphatic having 1-10 heteroatoms.

In some embodiments, R is optionally substituted 5-30 (5, 6, 7, 8, 9, 10, 11, 12, 13, 14, 15, 16, 17, 18, 19, 20, 21, 22, 23, 24, 25, 26, 27, 28, 29 or 30) membered heteroaryl having 1-10 heteroatoms. In some embodiments, R is optionally substituted 5-20 membered heteroaryl having 1-10 heteroatoms. In some embodiments, R is optionally substituted 5-10 membered heteroaryl having 1-10 heteroatoms. In some embodiments, R is optionally substituted 5-membered heteroaryl having 1-5 heteroatoms. In some embodiments, R is optionally substituted 5-membered heteroaryl having 1-4 heteroatoms. In some embodiments, R is optionally substituted 5-membered heteroaryl having 1-3 heteroatoms. In some embodiments, R is optionally substituted 5-membered heteroaryl having 1-2 heteroatoms. In some embodiments, R is optionally substituted 5-membered heteroaryl having one heteroatom. In some embodiments, R is optionally substituted 6-membered heteroaryl having 1-5 heteroatoms. In some embodiments, R is optionally substituted 6-membered heteroaryl having 1-4 heteroatoms. In some embodiments, R is optionally substituted 6-membered heteroaryl having 1-3 heteroatoms. In some embodiments, R is optionally substituted 6-membered heteroaryl having 1-2 heteroatoms. In some embodiments, R is optionally substituted 6-membered heteroaryl having one heteroatom. In some embodiments, R is optionally substituted monocyclic heteroaryl. In some embodiments, R is optionally substituted bicyclic heteroaryl. In some embodiments, R is optionally substituted polycyclic heteroaryl. In some embodiments, a heteroatom is nitrogen.

In some embodiments, R is optionally substituted 2-pyridinyl. In some embodiments, R is optionally substituted 3-pyridinyl. In some embodiments, R is optionally substituted 4-pyridinyl. In some embodiments, R is optionally substituted

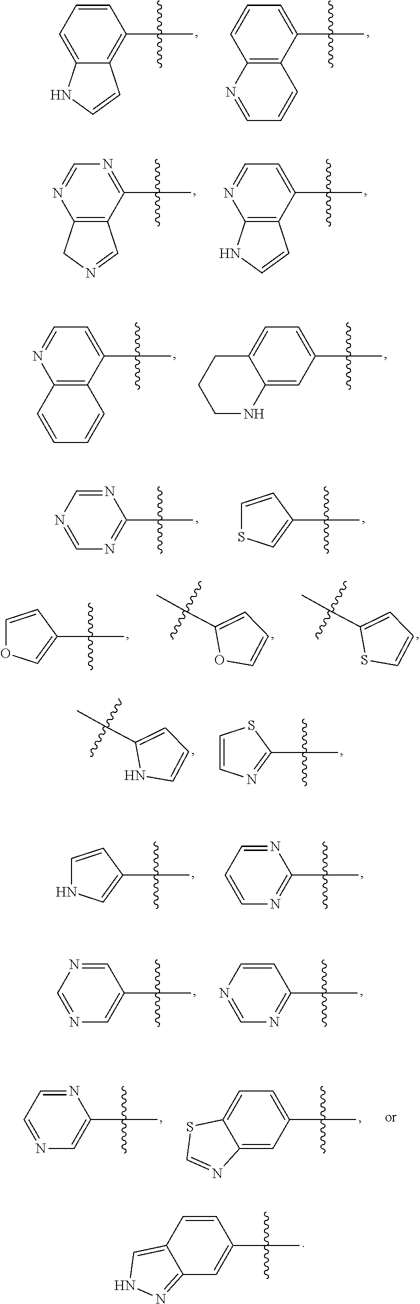

In some embodiments, R is optionally substituted 3-30 (3, 4, 5, 6, 7, 8, 9, 10, 11, 12, 13, 14, 15, 16, 17, 18, 19, 20, 21, 22, 23, 24, 25, 26, 27, 28, 29 or 30) membered heterocyclyl having 1-10 heteroatoms. In some embodiments, R is optionally substituted 3-membered heterocyclyl having 1-2 heteroatoms. In some embodiments, R is optionally substituted 4-membered heterocyclyl having 1-2 heteroatoms. In some embodiments, R is optionally substituted 5-20 membered heterocyclyl having 1-10 heteroatoms. In some embodiments, R is optionally substituted 5-10 membered heterocyclyl having 1-10 heteroatoms. In some embodiments, R is optionally substituted 5-membered heterocyclyl having 1-5 heteroatoms. In some embodiments, R is optionally substituted 5-membered heterocyclyl having 1-4 heteroatoms. In some embodiments, R is optionally substituted 5-membered heterocyclyl having 1-3 heteroatoms. In some embodiments, R is optionally substituted 5-membered heterocyclyl having 1-2 heteroatoms. In some embodiments, R is optionally substituted 5-membered heterocyclyl having one heteroatom. In some embodiments, R is optionally substituted 6-membered heterocyclyl having 1-5 heteroatoms. In some embodiments, R is optionally substituted 6-membered heterocyclyl having 1-4 heteroatoms. In some embodiments, R is optionally substituted 6-membered heterocyclyl having 1-3 heteroatoms. In some embodiments, R is optionally substituted 6-membered heterocyclyl having 1-2 heteroatoms. In some embodiments, R is optionally substituted 6-membered heterocyclyl having one heteroatom. In some embodiments, R is optionally substituted monocyclic heterocyclyl. In some embodiments, R is optionally substituted bicyclic heterocyclyl. In some embodiments, R is optionally substituted polycyclic heterocyclyl. In some embodiments, R is optionally substituted saturated heterocyclyl. In some embodiments, R is optionally substituted partially unsaturated heterocyclyl. In some embodiments, a heteroatom is nitrogen. In some embodiments, R is optionally substituted

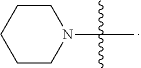 [SMILES: *N1CCCCC1]

In some embodiments, R is optionally substituted

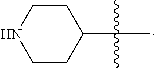 [SMILES: *C1CCNCC1]

In some embodiments, R is optionally substituted

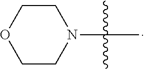 [SMILES: *N1CCOCC1]

In some embodiments, two R groups are optionally and independently taken together to form a covalent bond. In some embodiments, two or more R groups on the same atom are optionally and independently taken together with the atom to form an optionally substituted, 3-30 membered, monocyclic, bicyclic or polycyclic ring having, in addition to the atom, 0-10 heteroatoms. In some embodiments, two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms.

Various variables may comprise an optionally substituted ring, or can be taken together with their intervening atom(s) to form a ring. In some embodiments, a ring is 3-30 (e.g., 3, 4, 5, 6, 7, 8, 9, 10, 11, 12, 13, 14, 15, 16, 17, 18, 19, 20, 21, 22, 23, 24, 25, 26, 27, 28, 29 or 30) membered. In some embodiments, a ring is 3-20 membered. In some embodiments, a ring is 3-15 membered. In some embodiments, a ring is 3-10 membered. In some embodiments, a ring is 3-8 membered. In some embodiments, a ring is 3-7 membered. In some embodiments, a ring is 3-6 membered. In some embodiments, a ring is 4-20 membered. In some embodiments, a ring is 5-20 membered. In some embodiments, a ring is monocyclic. In some embodiments, a ring is bicyclic. In some embodiments, a ring is polycyclic. In some embodiments, each monocyclic ring or each monocyclic ring unit in bicyclic or polycyclic rings is independently saturated, partially saturated or aromatic. In some embodiments, each monocyclic ring or each monocyclic ring unit in bicyclic or polycyclic rings is independently 3-10 membered and has 0-5 heteroatoms.

In some embodiments, each heteroatom is independently selected oxygen, nitrogen, sulfur, silicon, and phosphorus. In some embodiments, each heteroatom is independently selected oxygen, nitrogen, sulfur, and phosphorus. In some embodiments, each heteroatom is independently selected oxygen, nitrogen, and sulfur. In some embodiments, a heteroatom is in an oxidized form.

As appreciated by those skilled in the art, many other types of internucleotidic linkages may be utilized in accordance with the present disclosure, for example, those described in U.S. Pat. Nos. 3,687,808; 4,469,863; 4,476,301; 5,177,195; 5,023,243; 5,034,506; 5,166,315; 5,185,444; 5,188,897; 5,214,134; 5,216,141; 5,235,033; 5,264,423; 5,264,564; 5,276,019; 5,278,302; 5,286,717; 5,321,131; 5,399,676; 5,405,938; 5,405,939; 5,434,257; 5,453,496; 5,455,233; 5,466,677; 5,466,677; 5,470,967; 5,476,925; 5,489,677; 5,519,126; 5,536,821; 5,541,307; 5,541,316; 5,550,111; 5,561,225; 5,563,253; 5,571,799; 5,587,361; 5,596,086; 5,602,240; 5,608,046; 5,610,289; 5,618,704; 5,623,070; 5,625,050; 5,633,360; 5,64,562; 5,663,312; 5,677,437; 5,677,439; 6,160,109; 6,239,265; 6,028,188; 6,124,445; 6,169,170; 6,172,209; 6,277,603; 6,326,199; 6,346,614; 6,444,423; 6,531,590; 6,534,639; 6,608,035; 6,683,167; 6,858,715; 6,867,294; 6,878,805; 7,015,315; 7,041,816; 7,273,933; 7,321,029; or RE39464. In certain embodiments, a modified internucleotidic linkage is one described in U.S. Pat. No. 9,982,257, US 20170037399, US 20180216108, WO 2017192664, WO 2017015575, WO2017062862, WO 2018067973, WO 2017160741, WO 2017192679, WO 2017210647, WO 2018098264, PCT/US18/35687, PCT/US18/38835, or PCT/US18/51398, the nucleobases, sugars, internucleotidic linkages, chiral auxiliaries/reagents, and technologies for oligonucleotide synthesis (reagents, conditions, cycles, etc.) of each of which is independently incorporated herein by reference.

In certain embodiments, each internucleotidic linkage in a ds oligonucleotide is independently selected from a natural phosphate linkage, a phosphorothioate linkage, and anon-negatively charged internucleotidic linkage (e.g., n001). In certain embodiments, each internucleotidic linkage in a ds oligonucleotide is independently selected from a natural phosphate linkage, a phosphorothioate linkage, and a neutral internucleotidic linkage (e.g., n001).

In certain embodiments, a ds oligonucleotide comprises one or more nucleotides that independently comprise a phosphorus modification prone to “autorelease” under certain conditions. That is, under certain conditions, a particular phosphorus modification is designed such that it self-cleaves from the ds oligonucleotide to provide, e.g., a natural phosphate linkage. In certain embodiments, such a phosphorus modification has a structure of —O-L-R¹, wherein L is L^{B} as described herein, and R¹ is R′ as described herein. In certain embodiments, a phosphorus modification has a structure of —S-L-R¹, wherein each L and R¹ is independently as described in the present disclosure. Certain examples of such phosphorus modification groups can be found in U.S. Pat. No. 9,982,257. In certain embodiments, an autorelease group comprises a morpholino group. In certain embodiments, an autorelease group is characterized by the ability to deliver an agent to the internucleotidic phosphorus linker, which agent facilitates further modification of the phosphorus atom such as, e.g., desulfurization. In certain embodiments, the agent is water and the further modification is hydrolysis to form a natural phosphate linkage.

In certain embodiments, a ds oligonucleotide comprises one or more internucleotidic linkages that improve one or more pharmaceutical properties and/or activities of the oligonucleotide. It is well documented in the art that certain oligonucleotides are rapidly degraded by nucleases and exhibit poor cellular uptake through the cytoplasmic cell membrane (Poijarvi-Virta et al., Curr. Med. Chem. (2006), 13(28); 3441-65; Wagner et al., Med. Res. Rev. (2000), 20(6):417-51; Peyrottes et al., Mini Rev. Med. Chem. (2004), 4(4):395-408; Gosselin et al., (1996), 43(1):196-208; Bologna et al., (2002), Antisense & Nucleic Acid Drug Development 12:33-41). Vives et al. (Nucleic Acids Research (1999), 27(20):4071-76) reported that tert-butyl SATE pro-oligonucleotides displayed markedly increased cellular penetration compared to the parent oligonucleotide under certain conditions.

Ds oligonucleotides can comprise various number of natural phosphate linkages. In certain embodiments, 5% or more of the internucleotidic linkages of provided ds oligonucleotides are natural phosphate linkages. In certain embodiments, 10% or more of the internucleotidic linkages of provided ds oligonucleotides are natural phosphate linkages. In certain embodiments, 15% or more of the internucleotidic linkages of provided ds oligonucleotides are natural phosphate linkages. In certain embodiments, 20% or more of the internucleotidic linkages of provided ds oligonucleotides are natural phosphate linkages. In certain embodiments, 25% or more of the internucleotidic linkages of provided ds oligonucleotides are natural phosphate linkages. In certain embodiments, 30% or more of the internucleotidic linkages of provided ds oligonucleotides are natural phosphate linkages. In certain embodiments, 35% or more of the internucleotidic linkages of provided ds oligonucleotides are natural phosphate linkages. In certain embodiments, 40% or more of the internucleotidic linkages of provided ds oligonucleotides are natural phosphate linkages. In certain embodiments, provided ds oligonucleotides comprises 1, 2, 3, 4, 5, 6, 7, 8, 9, 10 or more natural phosphate linkages. In certain embodiments, provided ds oligonucleotides comprises 4, 5, 6, 7, 8, 9, 10 or more natural phosphate linkages. In certain embodiments, the number of natural phosphate linkages is 2. In certain embodiments, the number of natural phosphate linkages is 3. In certain embodiments, the number of natural phosphate linkages is 4. In certain embodiments, the number of natural phosphate linkages is 5. In certain embodiments, the number of natural phosphate linkages is 6. In certain embodiments, the number of natural phosphate linkages is 7. In certain embodiments, the number of natural phosphate linkages is 8. In certain embodiments, some or all of the natural phosphate linkages are consecutive.

In certain embodiments, the present disclosure demonstrates that, in at least some cases, Sp internucleotidic linkages, among other things, at the 5′- and/or 3′-end can improve ds oligonucleotide stability. In certain embodiments, the present disclosure demonstrates that, among other things, natural phosphate linkages and/or Rp internucleotidic linkages may improve removal of ds oligonucleotides from a system. As appreciated by a person having ordinary skill in the art, various assays known in the art can be utilized to assess such properties in accordance with the present disclosure.

In certain embodiments, each phosphorothioate internucleotidic linkage in a ds oligonucleotide or a portion thereof (e.g., a domain, a subdomain, etc.) is independently chirally controlled. In certain embodiments, each is independently Sp or Rp. In certain embodiments, a high level is Sp as described herein. In certain embodiments, each phosphorothioate internucleotidic linkage in a ds oligonucleotide or a portion thereof is chirally controlled and is Sp. In certain embodiments, one or more, e.g., about 1-5 (e.g., about 1, 2, 3, 4, or 5) is Rp.

In certain embodiments, as illustrated in certain examples, a ds oligonucleotide or a portion thereof comprises one or more non-negatively charged internucleotidic linkages, each of which is optionally and independently chirally controlled. In certain embodiments, each non-negatively charged internucleotidic linkage is independently n001. In certain embodiments, a chiral non-negatively charged internucleotidic linkage is not chirally controlled. In certain embodiments, each chiral non-negatively charged internucleotidic linkage is not chirally controlled. In certain embodiments, a chiral non-negatively charged internucleotidic linkage is chirally controlled. In certain embodiments, a chiral non-negatively charged internucleotidic linkage is chirally controlled and is Rp. In certain embodiments, a chiral non-negatively charged internucleotidic linkage is chirally controlled and is Sp. In certain embodiments, each chiral non-negatively charged internucleotidic linkage is chirally controlled. In certain embodiments, the number of non-negatively charged internucleotidic linkages in a ds oligonucleotide or a portion thereof is about 1-10, or about 1, 2, 3, 4, 5, 6, 7, 8, 9, or 10. In certain embodiments, it is about 1. In certain embodiments, it is about 2. In certain embodiments, it is about 3. In certain embodiments, it is about 4. In certain embodiments, it is about 5. In certain embodiments, it is about 6. In certain embodiments, it is about 7. In certain embodiments, it is about 8. In certain embodiments, it is about 9. In certain embodiments, it is about 10. In certain embodiments, two or more non-negatively charged internucleotidic linkages are consecutive. In certain embodiments, no two non-negatively charged internucleotidic linkages are consecutive. In certain embodiments, all non-negatively charged internucleotidic linkages in a ds oligonucleotide or a portion thereof are consecutive (e.g., 3 consecutive non-negatively charged internucleotidic linkages). In certain embodiments, a non-negatively charged internucleotidic linkage, or two or more (e.g., about 2, about 3, about 4 etc.) consecutive non-negatively charged internucleotidic linkages, are at the 3′-end of a ds oligonucleotide or a portion thereof. In certain embodiments, the last two or three or four internucleotidic linkages of a ds oligonucleotide or a portion thereof comprise at least one internucleotidic linkage that is not a non-negatively charged internucleotidic linkage. In certain embodiments, the last two or three or four internucleotidic linkages of a ds oligonucleotide or a portion thereof comprise at least one internucleotidic linkage that is not n001. In certain embodiments, the internucleotidic linkage linking the first two nucleosides of a ds oligonucleotide or a portion thereof is a non-negatively charged internucleotidic linkage. In certain embodiments, the internucleotidic linkage linking the last two nucleosides of a ds oligonucleotide or a portion thereof is a non-negatively charged internucleotidic linkage. In certain embodiments, the internucleotidic linkage linking the first two nucleosides of a ds oligonucleotide or a portion thereof is a phosphorothioate internucleotidic linkage. In certain embodiments, it is Sp. In certain embodiments, the internucleotidic linkage linking the last two nucleosides of a ds oligonucleotide or a portion thereof is a phosphorothioate internucleotidic linkage. In certain embodiments, it is Sp.

In certain embodiments, one or more chiral internucleotidic linkages are chirally controlled and one or more chiral internucleotidic linkages are not chirally controlled. In certain embodiments, each phosphorothioate internucleotidic linkage is independently chirally controlled, and one or more non-negatively charged internucleotidic linkage are not chirally controlled. In certain embodiments, each phosphorothioate internucleotidic linkage is independently chirally controlled, and each non-negatively charged internucleotidic linkage is not chirally controlled. In certain embodiments, the internucleotidic linkage between the first two nucleosides of a ds oligonucleotide is a non-negatively charged internucleotidic linkage. In certain embodiments, the internucleotidic linkage between the last two nucleosides are each independently a non-negatively charged internucleotidic linkage. In certain embodiments, both are independently non-negatively charged internucleotidic linkages. In certain embodiments, each non-negatively charged internucleotidic linkage is independently neutral internucleotidic linkage. In certain embodiments, each non-negatively charged internucleotidic linkage is independently n001.

In certain embodiments, a controlled level of ds oligonucleotides in a composition are desired ds oligonucleotides. In certain embodiments, of all ds oligonucleotides in a composition that share a common base sequence (e.g., a desired sequence for a purpose), or of all ds oligonucleotides in a composition, level of desired ds oligonucleotides (which may exist in various forms (e.g., salt forms) and typically differ only at non-chirally controlled internucleotidic linkages (various forms of the same stereoisomer can be considered the same for this purpose)) is about 5%-100%, 10%-100%, 20%-100%, 30%-100%, 40%-100%, 50%-100%, 60%-100%, 70%-100%, 80-100%, 90-100%, 95-100%, 50%-90%, about 5%, 10%, 15%, 20%, 25%, 30%, 35%, 40%, 45%, 50%, 55%, 60%, 65%, 70%, 75%, 80%, 85%, 90%, 95%, or 100%, or at least 5%, 10%, 15%, 20%, 25%, 30%, 35%, 40%, 45%, 50%, 55%, 60%, 65%, 70%, 75%, 80%, 85%, 90%, 95%, or 99%. In certain embodiments, a level is at least about 50%. In certain embodiments, a level is at least about 60%. In certain embodiments, a level is at least about 70%. In certain embodiments, a level is at least about 75%. In certain embodiments, a level is at least about 80%. In certain embodiments, a level is at least about 85%. In certain embodiments, a level is at least about 90%. In certain embodiments, a level is or is at least (DS)^{nc}, wherein DS is about 90%, 91%, 92%, 93%, 94%, 95%, 96%, 97%, 98%, 99% or 99.5% and nc is the number of chirally controlled internucleotidic linkages as described in the present disclosure (e.g., 1-50, 1-40, 1-30, 1-25, 1-20, 5-50, 5-40, 5-30, 5-25, 5-20, 1, 2, 3, 4, 5, 6, 7, 8, 9, 10, 11, 12, 13, 14, 15, 16, 17, 18, 19, 20, 21, 22, 23, 24, 25 or more). In certain embodiments, a level is or is at least (DS)^{nc}, wherein DS is 95%-100%.

Various types of internucleotidic linkages may be utilized in combination of other structural elements, e.g., sugars, to achieve desired ds oligonucleotide properties and/or activities. For example, the present disclosure routinely utilizes modified internucleotidic linkages and modified sugars, optionally with natural phosphate linkages and natural sugars, in designing ds oligonucleotides. In certain embodiments, the present disclosure provides a ds oligonucleotide comprising one or more modified sugars. In certain embodiments, the present disclosure provides a ds oligonucleotide comprising one or more modified sugars and one or more modified internucleotidic linkages, one or more of which are natural phosphate linkages.

### 2.3. Double Stranded Oligonucleotide Compositions

Among other things, the present disclosure provides various ds oligonucleotide compositions. In certain embodiments, the present disclosure provides ds oligonucleotide compositions of ds oligonucleotides described herein. In certain embodiments, a ds oligonucleotide composition, e.g., a dsRNAi oligonucleotide composition, comprises a plurality of a ds oligonucleotide described in the present disclosure. In certain embodiments, a ds oligonucleotide composition, e.g., a dsRNAi oligonucleotide composition, is chirally controlled. In certain embodiments, a ds oligonucleotide composition, e.g., a dsRNAi oligonucleotide composition, is not chirally controlled (stereorandom).

Linkage phosphorus of natural phosphate linkages is achiral. Linkage phosphorus of many modified internucleotidic linkages, e.g., phosphorothioate internucleotidic linkages, are chiral. In certain embodiments, during preparation of ds oligonucleotide compositions (e.g., in traditional phosphoramidite ds oligonucleotide synthesis), configurations of chiral linkage phosphorus are not purposefully designed or controlled, creating non-chirally controlled (stereorandom) ds oligonucleotide compositions (substantially racemic preparations) which are complex, random mixtures of various stereoisomers (diastereoisomers)—for ds oligonucleotides with n chiral internucleotidic linkages (linkage phosphorus being chiral), typically 2ⁿ stereoisomers (e.g., when n is 10, 2¹⁰=1,032; when n is 20, 2²⁰=1,048,576). These stereoisomers have the same constitution, but differ with respect to the pattern of stereochemistry of their linkage phosphorus.

In certain embodiments, stereorandom ds oligonucleotide compositions have sufficient properties and/or activities for certain purposes and/or applications. In certain embodiments, stereorandom ds oligonucleotide compositions can be cheaper, easier and/or simpler to produce than chirally controlled ds oligonucleotide compositions. However, stereoisomers within stereorandom compositions may have different properties, activities, and/or toxicities, resulting in inconsistent therapeutic effects and/or unintended side effects by stereorandom compositions, particularly compared to certain chirally controlled ds oligonucleotide compositions of ds oligonucleotides of the same constitution.

### 2.3.1. Chirally Controlled Double Stranded Oligonucleotide Compositions

In certain embodiments, the present disclosure encompasses technologies for designing and preparing chirally controlled ds oligonucleotide compositions. In certain embodiments, a chirally controlled ds oligonucleotide composition comprises a controlled/pre-determined (not random as in stereorandom compositions) level of a plurality of ds oligonucleotides, wherein the ds oligonucleotides share the same linkage phosphorus stereochemistry at one or more chiral internucleotidic linkages (chirally controlled internucleotidic linkages). In certain embodiments, ds oligonucleotides of a plurality share the same pattern of backbone chiral centers (stereochemistry of linkage phosphorus). In certain embodiments, a pattern of backbone chiral centers is as described in the present disclosure. In certain embodiments, ds oligonucleotides of a plurality share a common constitution. In certain embodiments, they are structurally identical.

For example, in certain embodiments, the present disclosure provides a ds oligonucleotide composition comprising a plurality of ds oligonucleotides, wherein ds oligonucleotides of the plurality share:
1) a common base sequence, and
2) the same linkage phosphorus stereochemistry independently at one or more (e.g., about 1-50, 1-40, 1-30, 1-25, 1-20, 1-15, 1-10, 5-50, 5-40, 5-30, 5-25, 5-20, 5-15, 5-10, 1, 2, 3, 4, 5, 6, 7, 8, 9, 10, 11, 12, 13, 14, 15, 16, 17, 18, 19, 20, 21, 22, 23, 24, or 25 or more) chiral internucleotidic linkages (“chirally controlled internucleotidic linkages”); wherein level of ds oligonucleotides of the plurality in the composition is non-random (e.g., controlled/pre-determined as described herein).

In certain embodiments, the present disclosure provides a ds oligonucleotide composition comprising a plurality of ds oligonucleotides, wherein ds oligonucleotides of the plurality share:
1) a common base sequence, and
2) the same linkage phosphorus stereochemistry independently at one or more (e.g., about 1-50, 1-40, 1-30, 1-25, 1-20, 1-15, 1-10, 5-50, 5-40, 5-30, 5-25, 5-20, 5-15, 5-10, 1, 2, 3, 4, 5, 6, 7, 8, 9, 10, 11, 12, 13, 14, 15, 16, 17, 18, 19, 20, 21, 22, 23, 24, or 25 or more) chiral internucleotidic linkages (“chirally controlled internucleotidic linkages”); wherein the composition is enriched relative to a substantially racemic preparation of ds oligonucleotides sharing the common base sequence for oligonucleotides of the plurality.

In certain embodiments, the present disclosure provides a ds oligonucleotide composition comprising a plurality of ds oligonucleotides, wherein ds oligonucleotides of the plurality share:
1) a common base sequence, and
2) the same linkage phosphorus stereochemistry independently at one or more (e.g., about 1-50, 1-40, 1-30, 1-25, 1-20, 1-15, 1-10, 5-50, 5-40, 5-30, 5-25, 5-20, 5-15, 5-10, 1, 2, 3, 4, 5, 6, 7, 8, 9, 10, 11, 12, 13, 14, 15, 16, 17, 18, 19, 20, 21, 22, 23, 24, or 25 or more) chiral internucleotidic linkages (“chirally controlled internucleotidic linkages”); wherein about 1%-100%, (e.g., about 5%-100%, 10%-100%, 20%-100%, 30%-100%, 40%-100%, 50%-100%, 60%-100%, 70%-100%, 80-100%, 90-100%, 95-100%, 50%-90%, or about 5%, 10%, 20%, 30%, 40%, 50%, 60%, 70%, 80%, 85%, 90%, 91%, 92%, 93%, 94%, 95%, 96%, 97%, 98%, 99%, or 100%, or at least 5%, 10%, 20%, 30%, 40%, 50%, 60%, 70%, 80%, 85%, 90%, 91%, 92%, 93%, 94%, 95%, 96%, 97%, 98%, or 99%) of all ds oligonucleotides in the composition that share the common base sequence are ds oligonucleotides of the plurality.

In certain embodiments, the percentage/level of the ds oligonucleotides of a plurality is or is at least (DS)^{nc}, wherein DS is 90%-100%, and nc is the number of chirally controlled internucleotidic linkages. In certain embodiments, nc is 5, 6, 7, 8, 9, 10 or more. In certain embodiments, a percentage/level is at least 10%.

In certain embodiments, a percentage/level is at least 20%. In certain embodiments, a percentage/level is at least 30%. In certain embodiments, a percentage/level is at least 40%. In certain embodiments, a percentage/level is at least 50%. In certain embodiments, a percentage/level is at least 60%. In certain embodiments, a percentage/level is at least 65%. In certain embodiments, a percentage/level is at least 70%. In certain embodiments, a percentage/level is at least 75%. In certain embodiments, a percentage/level is at least 80%. In certain embodiments, a percentage/level is at least 85%. In certain embodiments, a percentage/level is at least 90%. In certain embodiments, a percentage/level is at least 95%.

In certain embodiments, ds oligonucleotides of a plurality share a common pattern of backbone linkages. In certain embodiments, each ds oligonucleotide of a plurality independently has an internucleotidic linkage of a particular constitution (e.g., —O—P(O)(SH)—O—) or a salt form thereof (e.g., —O—P(O)(SNa)—O—) independently at each internucleotidic linkage site. In certain embodiments, internucleotidic linkages at each internucleotidic linkage site are of the same form. In certain embodiments, internucleotidic linkages at each internucleotidic linkage site are of different forms.

In certain embodiments, ds oligonucleotides of a plurality share a common constitution. In certain embodiments, ds oligonucleotides of a plurality are of the same form of a common constitution. In certain embodiments, ds oligonucleotides of a plurality are of two or more forms of a common constitution. In certain embodiments, ds oligonucleotides of a plurality are each independently of a particular oligonucleotide or a pharmaceutically acceptable salt thereof, or of a ds oligonucleotide having the same constitution as the particular ds oligonucleotide or a pharmaceutically acceptable salt thereof. In certain embodiments, about 1%-100%, (e.g., about 5%-100%, 10%-100%, 20%-100%, 30%-100%, 40%-100%, 50%-100%, 60%-100%, 70%-100%, 80-100%, 90-100%, 95-100%, 50%-90%, or about 5%, 10%, 20%, 30%, 40%, 50%, 60%, 70%, 80%, 85%, 90%, 91%, 92%, 93%, 94%, 95%, 96%, 97%, 98%, 99%, or 100%, or at least 5%, 10%, 20%, 30%, 40%, 50%, 60%, 70%, 80%, 85%, 90%, 91%, 92%, 93%, 94%, 95%, 96%, 97%, 98%, or 99%) of all ds oligonucleotides in the composition that share a common constitution are ds oligonucleotides of the plurality. In certain embodiments, a percentage of a level is or is at least (DS)^{nc}, wherein DS is 90%-100%, and nc is the number of chirally controlled internucleotidic linkages. In certain embodiments, nc is 5, 6, 7, 8, 9, 10 or more. In certain embodiments, a level is at least 10%. In certain embodiments, a level is at least 20%. In certain embodiments, a level is at least 30%. In certain embodiments, a level is at least 40%. In certain embodiments, a level is at least 50%. In certain embodiments, a level is at least 60%. In certain embodiments, a level is at least 65%. In certain embodiments, a level is at least 70%. In certain embodiments, a level is at least 75%. In certain embodiments, a level is at least 80%. In certain embodiments, a level is at least 85%. In certain embodiments, a level is at least 90%. In certain embodiments, a level is at least 95%.

In certain embodiments, each phosphorothioate internucleotidic linkage is independently a chirally controlled internucleotidic linkage.

In certain embodiments, the present disclosure provides a chirally controlled ds oligonucleotide composition comprising a plurality of ds oligonucleotides of a particular ds oligonucleotide type characterized by:
a) a common base sequence;
b) a common pattern of backbone linkages;
c) a common pattern of backbone chiral centers; wherein the composition is enriched, relative to a substantially racemic preparation of ds oligonucleotides having the same common base sequence, for ds oligonucleotides of the particular oligonucleotide type.

In certain embodiments, the present disclosure provides a chirally controlled ds oligonucleotide composition comprising a plurality of ds oligonucleotides of a particular ds oligonucleotide type characterized by:
a) a common base sequence;
b) a common pattern of backbone linkages;
c) a common pattern of backbone chiral centers; wherein ds oligonucleotides of the plurality comprise at least one internucleotidic linkage comprising a common linkage phosphorus in the Sp configuration; wherein the composition is enriched, relative to a substantially racemic preparation of d oligonucleotides having the same common base sequence, for ds oligonucleotides of the particular ds oligonucleotide type.

Common patterns of backbone chiral centers, as appreciated by those skilled in the art, comprise at least one Rp or at least one Sp. Certain patterns of backbone chiral centers are illustrated in, e.g., Table 1.

In certain embodiments, a chirally controlled ds oligonucleotide composition is enriched, relative to a substantially racemic preparation of ds oligonucleotides share the same common base sequence and a common pattern of backbone linkages, for ds oligonucleotides of the particular ds oligonucleotide type.

In certain embodiments, ds oligonucleotides of a plurality, e.g., a particular ds oligonucleotide type, have a common pattern of backbone phosphorus modifications and a common pattern of nucleoside modifications. In certain embodiments, ds oligonucleotides of a plurality have a common pattern of sugar modifications. In certain embodiments, ds oligonucleotides of a plurality have a common pattern of base modifications. In certain embodiments, ds oligonucleotides of a plurality have a common pattern of nucleoside modifications. In certain embodiments, ds oligonucleotides of a plurality have the same constitution. In certain embodiments, ds oligonucleotides of a plurality are identical. In certain embodiments, ds oligonucleotides of a plurality are of the same ds oligonucleotide (as those skilled in the art will appreciate, such ds oligonucleotides may each independently exist in one of the various forms of the ds oligonucleotide, and may be the same, or different forms of the ds oligonucleotide). In certain embodiments, ds oligonucleotides of a plurality are each independently of the same ds oligonucleotide or a pharmaceutically acceptable salt thereof.

In certain embodiments, the present disclosure provides chirally controlled ds oligonucleotide compositions, e.g., of many oligonucleotides in Table 1, whose “stereochemistry/linkage” contain S and/or R. In certain embodiments, ds oligonucleotides of a plurality are each independently a particular ds oligonucleotide in Table 1 whose “stereochemistry/linkage” contains S and/or R, optionally in various forms. In certain embodiments, ds oligonucleotides of a plurality are each independently a particular ds oligonucleotide in Table 1, whose “stereochemistry/linkage” contains S and/or R, or a pharmaceutically acceptable salt thereof.

In certain embodiments, level of a plurality of ds oligonucleotides in a composition can be determined as the product of the diastereopurity of each chirally controlled internucleotidic linkage in the ds oligonucleotides. In certain embodiments, diastereopurity of an internucleotidic linkage connecting two nucleosides in a ds oligonucleotide (or nucleic acid) is represented by the diastereopurity of an internucleotidic linkage of a dimer connecting the same two nucleosides, wherein the dimer is prepared using comparable conditions, in some instances, identical synthetic cycle conditions.

In certain embodiments, all chiral internucleotidic linkages are independently chiral controlled, and the composition is a completely chirally controlled ds oligonucleotide composition. In certain embodiments, not all chiral internucleotidic linkages are chiral controlled internucleotidic linkages, and the composition is a partially chirally controlled ds oligonucleotide composition.

Ds oligonucleotides may comprise or consist of various patterns of backbone chiral centers (patterns of stereochemistry of chiral linkage phosphorus). Certain useful patterns of backbone chiral centers are described in the present disclosure. In certain embodiments, a plurality of ds oligonucleotides share a common pattern of backbone chiral centers, which is or comprises a pattern described in the present disclosure (e.g., as in “Stereochemistry and Patterns of Backbone Chiral Centers”, a pattern of backbone chiral centers of a chirally controlled ds oligonucleotide in Table 1, etc.).

In certain embodiments, a chirally controlled ds oligonucleotide composition is chirally pure (or stereopure, stereochemically pure) ds oligonucleotide composition, wherein the ds oligonucleotide composition comprises a plurality of ds oligonucleotides, wherein the ds oligonucleotides are independently of the same stereoisomer (including that each chiral element of the ds oligonucleotides, including each chiral linkage phosphorus, is independently defined (stereodefined)). A chirally pure (or stereopure, stereochemically pure) ds oligonucleotide composition of a ds oligonucleotide stereoisomer does not contain other stereoisomers (as appreciated by those skilled in the art, one or more unintended stereoisomers may exist as impurities from, e.g., preparation, storage, etc.).

### 2.3.2 Stereochemistry and Patterns of Backbone Chiral Centers

In contrast to natural phosphate linkages, linkage phosphorus of chiral modified internucleotidic linkages, e.g., phosphoryl guanidine or phosphorothioate internucleotidic linkages, are chiral. Among other things, the present disclosure provides technologies (e.g., oligonucleotides, compositions, methods, etc.) comprising control of stereochemistry of chiral linkage phosphorus in chiral internucleotidic linkages. In certain embodiments, as demonstrated herein, control of stereochemistry can provide improved properties and/or activities, including desired stability, reduced toxicity, improved reduction of target nucleic acids, etc. In certain embodiments, the present disclosure provides useful patterns of backbone chiral centers for oligonucleotides and/or regions thereof, which pattern is a combination of stereochemistry of each chiral linkage phosphorus (Rp or Sp) of chiral linkage phosphorus, indication of each achiral linkage phosphorus (Op, if any), etc. from 5′ to 3′. In certain embodiments, patterns of backbone chiral centers can control cleavage patterns of target nucleic acids when they are contacted with provided ds oligonucleotides or compositions thereof in a cleavage system (e.g., in vitro assay, cells, tissues, organs, organisms, subjects, etc.). In certain embodiments, patterns of backbone chiral centers improve cleavage efficiency and/or selectivity of target nucleic acids when they are contacted with provided ds oligonucleotides or compositions thereof in a cleavage system.

In certain embodiments, a pattern of backbone chiral centers of a ds oligonucleotide or a region thereof comprises or is any (Np)n(Op)m, wherein Np is Rp or Sp, Op represents a linkage phosphorus being achiral (e.g., as for the linkage phosphorus of natural phosphate linkages), and each of n and m is independently as defined and described in the present disclosure. In certain embodiments, a pattern of backbone chiral centers of a ds oligonucleotide or a region thereof comprises or is (Sp)n(Op)m, wherein each variable is independently as defined and described in the present disclosure. In certain embodiments, a pattern of backbone chiral centers of a ds oligonucleotide or a region thereof comprises or is (Rp)n(Op)m, wherein each variable is independently as defined and described in the present disclosure. In certain embodiments, n is 1. In certain embodiments, a pattern of backbone chiral centers of a ds oligonucleotide or a region thereof comprises or is (Sp)(Op)m, wherein m is 1, 2, 3, 4, 5, 6, 7, 8, 9, or 10. In certain embodiments, a pattern of backbone chiral centers of an oligonucleotide or a region thereof comprises or is (Rp)(Op)m, wherein m is 1, 2, 3, 4, 5, 6, 7, 8, 9, or 10. In certain embodiments, the pattern of backbone chiral centers of a 5′-wing is or comprises (Np)n(Op)m. In certain embodiments, the pattern of backbone chiral centers of a 5′-wing is or comprises (Sp)n(Op)m. In certain embodiments, the pattern of backbone chiral centers of a 5′-wing is or comprises (Rp)n(Op)m. In certain embodiments, the pattern of backbone chiral centers of a 5′-wing is or comprises (Sp)(Op)m. In certain embodiments, the pattern of backbone chiral centers of a 5′-wing is or comprises (Rp)(Op)m. In certain embodiments, the pattern of backbone chiral centers of a 5′-wing is (Sp)(Op)m. In certain embodiments, the pattern of backbone chiral centers of a 5′-wing is (Rp)(Op)m. In certain embodiments, the pattern of backbone chiral centers of a 5′-wing is (Sp)(Op)m, wherein Sp is the linkage phosphorus configuration of the first internucleotidic linkage of the oligonucleotide from the 5′-end. In certain embodiments, the pattern of backbone chiral centers of a 5′-wing is (Rp)(Op)m, wherein Rp is the linkage phosphorus configuration of the first internucleotidic linkage of the oligonucleotide from the 5′-end. In certain embodiments, as described in the present disclosure, m is 2; in certain embodiments, m is 3; in certain embodiments, m is 4; in certain embodiments, m is 5; in certain embodiments, m is 6.

In certain embodiments, a pattern of backbone chiral centers of a ds oligonucleotide or a region thereof comprises or is (Op)m(Np)n, wherein Np is Rp or Sp, Op represents a linkage phosphorus being achiral (e.g., as for the linkage phosphorus of natural phosphate linkages), and each of n and m is independently as defined and described in the present disclosure. In certain embodiments, a pattern of backbone chiral centers of an oligonucleotide or a region thereof comprises or is (Op)m(Sp)n, wherein each variable is independently as defined and described in the present disclosure. In certain embodiments, a pattern of backbone chiral centers of a ds oligonucleotide or a region thereof comprises or is (Op)m(Rp)n, wherein each variable is independently as defined and described in the present disclosure. In certain embodiments, n is 1. In certain embodiments, a pattern of backbone chiral centers of a ds oligonucleotide or a region thereof comprises or is (Op)m(Sp), wherein m is 1, 2, 3, 4, 5, 6, 7, 8, 9, or 10. In certain embodiments, a pattern of backbone chiral centers of an oligonucleotide or a region thereof comprises or is (Op)m(Rp), wherein m is 1, 2, 3, 4, 5, 6, 7, 8, 9, or 10. In certain embodiments, the pattern of backbone chiral centers of a 3′-wing is or comprises (Op)m(Np)n. In certain embodiments, the pattern of backbone chiral centers of a 3′-wing is or comprises (Op)m(Sp)n. In certain embodiments, the pattern of backbone chiral centers of a 3′-wing is or comprises (Op)m(Rp)n. In certain embodiments, the pattern of backbone chiral centers of a 3′-wing is or comprises (Op)m(Sp). In certain embodiments, the pattern of backbone chiral centers of a 3′-wing is or comprises (Op)m(Rp). In certain embodiments, the pattern of backbone chiral centers of a 3′-wing is (Op)m(Sp). In certain embodiments, the pattern of backbone chiral centers of a 3′-wing is (Op)m(Rp). In certain embodiments, the pattern of backbone chiral centers of a 3′-wing is (Op)m(Sp), wherein Sp is the linkage phosphorus configuration of the last internucleotidic linkage of the ds oligonucleotide from the 5′-end. In certain embodiments, the pattern of backbone chiral centers of a 3′-wing is (Op)m(Rp), wherein Rp is the linkage phosphorus configuration of the last internucleotidic linkage of the oligonucleotide from the 5′-end. In certain embodiments, as described in the present disclosure, m is 2; in certain embodiments, m is 3; in certain embodiments, m is 4; in certain embodiments, m is 5; in certain embodiments, m is 6.

In certain embodiments, a pattern of backbone chiral centers of a ds oligonucleotide or a region thereof (e.g., a core) comprises or is (Sp)m(Rp/Op)n or (Rp/Op)n(Sp)m, wherein each variable is independently as described in the present disclosure. In certain embodiments, a pattern of backbone chiral centers of a ds oligonucleotide or a region thereof (e.g., a core) comprises or is (Sp)m(Rp)n or (Rp)n(Sp)m, wherein each variable is independently as described in the present disclosure. In certain embodiments, a pattern of backbone chiral centers of a ds oligonucleotide or a region thereof (e.g., a core) comprises or is (Sp)m(Op)n or (Op)n(Sp)m, wherein each variable is independently as described in the present disclosure. In certain embodiments, a pattern of backbone chiral centers of a ds oligonucleotide or a region thereof (e.g., a core) comprises or is (Np)t[(Rp/Op)n(Sp)m]y or [(Rp/Op)n(Sp)m]y(Np)t, wherein y is 1-50, and each other variable is independently as described in the present disclosure. In certain embodiments, a pattern of backbone chiral centers of a ds oligonucleotide or a region thereof (e.g., a core) comprises or is (Np)t[(Rp)n(Sp)m]y or [(Rp)n(Sp)m]y(Np)t, wherein each variable is independently as described in the present disclosure. In certain embodiments, a pattern of backbone chiral centers of a ds n oligonucleotide or a region thereof (e.g., a core) comprises or is [(Rp/Op)n(Sp)m]y(Rp)k, [(Rp/Op)n(Sp)m]y, (Sp)t[(Rp/Op)n(Sp)m]y, (Sp)t[(Rp/Op)n(Sp)m]y(Rp)k, wherein k is 1-50, and each other variable is independently as described in the present disclosure. In certain embodiments, a pattern of backbone chiral centers of a ds oligonucleotide or a region thereof (e.g., a core) comprises or is [(Op)n(Sp)m]y(Rp)k, [(Op)n(Sp)m]y, (Sp)t[(Op)n(Sp)m]y, (Sp)t[(Op)n(Sp)m]y(Rp)k, wherein each variable is independently as described in the present disclosure. In certain embodiments, a pattern of backbone chiral centers of a ds oligonucleotide or a region thereof (e.g., a core) comprises or is [(Rp)n(Sp)m]y(Rp)k, [(Rp)n(Sp)m]y, (Sp)t[(Rp)n(Sp)m]y, (Sp)t[(Rp)n(Sp)m]y(Rp)k, wherein each variable is independently as described in the present disclosure. In certain embodiments, an oligonucleotide comprises a core region. In certain embodiments, an oligonucleotide comprises a core region, wherein each sugar in the core region does not contain a 2′-OR¹, wherein R¹ is as described in the present disclosure. In certain embodiments, a ds oligonucleotide comprises a core region, wherein each sugar in the core region is independently a natural DNA sugar. In certain embodiments, the pattern of backbone chiral centers of the core comprises or is (Rp)(Sp)m. In certain embodiments, the pattern of backbone chiral centers of the core comprises or is (Op)(Sp)m. In certain embodiments, the pattern of backbone chiral centers of the core comprises or is (Np)t[(Rp/Op)n(Sp)m]y or [(Rp/Op)n(Sp)m]y(Np)t. In certain embodiments, the pattern of backbone chiral centers of the core comprises or is (Np)t[(Rp/Op)n(Sp)m]y or [(Rp/Op)n(Sp)m]y(Np)t. In certain embodiments, the pattern of backbone chiral centers of the core comprises or is (Np)t[(Rp)n(Sp)m]y or [(Rp)n(Sp)m]y(Np)t. In certain embodiments, the pattern of backbone chiral centers of a core comprises or is [(Rp/Op)n(Sp)m]y(Rp)k, [(Rp/Op)n(Sp)m]y, (Sp)t[(Rp/Op)n(Sp)m]y, (Sp)t[(Rp/Op)n(Sp)m]y(Rp)k. In certain embodiments, a pattern of backbone chiral centers of a core comprises or is [(Op)n(Sp)m]y(Rp)k, [(Op)n(Sp)m]y, (Sp)t[(Op)n(Sp)m]y, (Sp)t[(Op)n(Sp)m]y(Rp)k. In certain embodiments, a pattern of backbone chiral centers of a core comprises or is [(Rp)n(Sp)m]y(Rp)k, [(Rp)n(Sp)m]y, (Sp)t[(Rp)n(Sp)m]y, or (Sp)t[(Rp)n(Sp)m]y(Rp)k. In certain embodiments, a pattern of backbone chiral centers of a core comprises [(Rp)n(Sp)m]y(Rp)k. In certain embodiments, a pattern of backbone chiral centers of a core comprises [(Rp)n(Sp)m]y(Rp). In certain embodiments, a pattern of backbone chiral centers of a core comprises [(Rp)n(Sp)m]y. In certain embodiments, a pattern of backbone chiral centers of a core comprises (Sp)t[(Rp)n(Sp)m]y. In certain embodiments, a pattern of backbone chiral centers of a core comprises (Sp)t[(Rp)n(Sp)m]y(Rp)k. In certain embodiments, a pattern of backbone chiral centers of a core comprises (Sp)t[(Rp)n(Sp)m]y(Rp). In certain embodiments, a pattern of backbone chiral centers of a core is [(Rp)n(Sp)m]y(Rp)k. In certain embodiments, a pattern of backbone chiral centers of a core is [(Rp)n(Sp)m]y(Rp). In certain embodiments, a pattern of backbone chiral centers of a core is [(Rp)n(Sp)m]y. In certain embodiments, a pattern of backbone chiral centers of a core is (Sp)t[(Rp)n(Sp)m]y. In certain embodiments, a pattern of backbone chiral centers of a core is (Sp)t[(Rp)n(Sp)m]y(Rp)k. In certain embodiments, a pattern of backbone chiral centers of a core is (Sp)t[(Rp)n(Sp)m]y(Rp). In certain embodiments, each n is 1. In certain embodiments, each t is 1. In certain embodiments, t is 2, 3, 4, 5, 6, 7, 8, 9, or 10. In certain embodiments, each of t and n is 1. In certain embodiments, each m is 2 or more. In certain embodiments, k is 1. In certain embodiments, k is 2-10.

In certain embodiments, a pattern of backbone chiral centers comprises or is (Sp)m(Rp)n, (Rp)n(Sp)m, (Np)t(Rp)n(Sp)m, (Sp)t(Rp)n(Sp)m, (Np)t[(Rp)n(Sp)m]2, (Sp)t[(Rp)n(Sp)m]2, (Np)t(Op)n(Sp)m, (Sp)t(Op)n(Sp)m, (Np)t[(Op)n(Sp)m]2, or (Sp)t[(Op)n(Sp)m]2. In certain embodiments, a pattern is (Np)t(Op/Rp)n(Sp)m(Op/Rp)n(Sp)m. In certain embodiments, a pattern is (Np)t(Op/Rp)n(Sp)1-5(Op/Rp)n(Sp)m. In certain embodiments, a pattern is (Np)t(Op/Rp)n(Sp)2-5(Op/Rp)n(Sp)m. In certain embodiments, a pattern is (Np)t(Op/Rp)n(Sp)2(Op/Rp)n(Sp)m. In certain embodiments, a pattern is (Np)t(Op/Rp)n(Sp)3(Op/Rp)n(Sp)m. In certain embodiments, a pattern is (Np)t(Op/Rp)n(Sp)4(Op/Rp)n(Sp)m. In certain embodiments, a pattern is (Np)t(Op/Rp)n(Sp)5(Op/Rp)n(Sp)m.

In certain embodiments, Np is Sp. In certain embodiments, (Op/Rp) is Op. In certain embodiments, (Op/Rp) is Rp. In certain embodiments, Np is Sp and (Op/Rp) is Rp. In certain embodiments, Np is Sp and (Op/Rp) is Op. In certain embodiments, Np is Sp and at least one (Op/Rp) is Rp, and at least one (Op/Rp) is Op. In certain embodiments, a pattern of backbone chiral centers comprises or is (Rp)n(Sp)m, (Np)t(Rp)n(Sp)m, or (Sp)t(Rp)n(Sp)m, wherein m>2. In certain embodiments, a pattern of backbone chiral centers comprises or is (Rp)n(Sp)m, (Np)t(Rp)n(Sp)m, or (Sp)t(Rp)n(Sp)m, wherein n is 1, at least one t>1, and at least one m>2.

In certain embodiments, oligonucleotides comprising core regions whose patterns of backbone chiral centers starting with Rp can provide high activities and/or improved properties. In certain embodiments, oligonucleotides comprising core regions whose patterns of backbone chiral centers ending with Rp can provide high activities and/or improved properties. In certain embodiments, oligonucleotides comprising core regions whose patterns of backbone chiral centers starting with Rp provide high activities (e.g., target cleavage) without significantly impacting its properties, e.g., stability. In certain embodiments, oligonucleotides comprising core regions whose patterns of backbone chiral centers ending with Rp provide high activities (e.g., target cleavage) without significantly impacting its properties, e.g., stability. In certain embodiments, patterns of backbone chiral centers start with Rp and end with Sp. In certain embodiments, patterns of backbone chiral centers start with Rp and end with Rp. In certain embodiments, patterns of backbone chiral centers start with Sp and end with Rp.

In certain embodiments, a pattern of backbone chiral centers of a RNAi oligonucleotide or a region thereof (e.g., a core) comprises or is (Op)[(Rp/Op)n(Sp)m]y(Rp)k(Op), (Op)[(Rp/Op)n(Sp)m]y(Op), (Op)(Sp)t[(Rp/Op)n(Sp)m]y(Op), or (Op)(Sp)t[(Rp/Op)n(Sp)m]y(Rp)k(Op), wherein k is 1-50, and each other variable is independently as described in the present disclosure. In certain embodiments, a pattern of backbone chiral centers of a RNAi oligonucleotide comprises or is (Op)[(Rp/Op)n(Sp)m]y(Rp)k(Op), (Op)[(Rp/Op)n(Sp)m]y(Op), (Op)(Sp)t[(Rp/Op)n(Sp)m]y(Op), or (Op)(Sp)t[(Rp/Op)n(Sp)m]y(Rp)k(Op), wherein each of f, g, h and j is independently 1-50, and each other variable is independently as described in the present disclosure, and the oligonucleotide comprises a core region whose pattern of backbone chiral centers comprises or is [(Rp/Op)n(Sp)m]y(Rp)k, [(Rp/Op)n(Sp)m]y, (Sp)t[(Rp/Op)n(Sp)m]y, or (Sp)t[(Rp/Op)n(Sp)m]y(Rp)k as described in the present disclosure. In certain embodiments, a pattern of backbone chiral centers is or comprises (Op)[(Rp/Op)n(Sp)m]y(Rp)k(Op). In certain embodiments, a pattern of backbone chiral centers is or comprises (Op)[(Rp/Op)n(Sp)m]y(Rp)(Op). In certain embodiments, a pattern of backbone chiral centers is or comprises (Op)[(Rp/Op)n(Sp)m]y(Op). In certain embodiments, a pattern of backbone chiral centers is or comprises (Op)(Sp)t[(Rp/Op)n(Sp)m]y(Op). In certain embodiments, a pattern of backbone chiral centers is or comprises (Op)(Sp)t[(Rp/Op)n(Sp)m]y(Rp)k(Op). In certain embodiments, a pattern of backbone chiral centers is or comprises (Op)(Sp)t[(Rp/Op)n(Sp)m]y(Rp)(Op). In certain embodiments, a pattern of backbone chiral centers is or comprises (Op)[(Rp)n(Sp)m]y(Rp)k(Op). In certain embodiments, a pattern of backbone chiral centers is or comprises (Op)[(Rp)n(Sp)m]y(Rp)(Op). In certain embodiments, a pattern of backbone chiral centers is or comprises (Op)[(Rp)n(Sp)m]y(Op). In certain embodiments, a pattern of backbone chiral centers is or comprises (Op)(Sp)t[(Rp)n(Sp)m]y(Op). In certain embodiments, a pattern of backbone chiral centers is or comprises (Op)(Sp)t[(Rp)n(Sp)m]y(Rp)k(Op). In certain embodiments, a pattern of backbone chiral centers is or comprises (Op)(Sp)t[(Rp)n(Sp)m]y(Rp)(Op). In certain embodiments, each n is 1. In certain embodiments, k is 1. In certain embodiments, k is 2-10.

In certain embodiments, a pattern of backbone chiral centers of a RNAi oligonucleotide or a region thereof (e.g., a core) comprises or is (Np)f(Op)g[(Rp/Op)n(Sp)m]y(Rp)k(Op)h(Np)j, (Np)f(Op)g[(Rp/Op)n(Sp)m]y(Op)h(Np)j, (Np)f(Op)g(Sp)t[(Rp/Op)n(Sp)m]y(Op)h(Np)j, or (Np)f(Op)g(Sp)t[(Rp/Op)n(Sp)m]y(Rp)k(Op)h(Np)j, wherein each of f, g, h and j is independently 1-50, and each other variable is independently as described in the present disclosure.

In certain embodiments, a pattern of backbone chiral centers of a RNAi oligonucleotide comprises or is (Np)f(Op)g[(Rp/Op)n(Sp)m]y(Rp)k(Op)h(Np)j, (Np)f(Op)g[(Rp/Op)n(Sp)m]y(Op)h(Np)j, (Np)f(Op)g(Sp)t[(Rp/Op)n(Sp)m]y(Op)h(Np)j, or (Np)f(Op)g(Sp)t[(Rp/Op)n(Sp)m]y(Rp)k(Op)h(Np)j, and the oligonucleotide comprises a core region whose pattern of backbone chiral centers comprises or is [(Rp/Op)n(Sp)m]y(Rp)k, [(Rp/Op)n(Sp)m]y, (Sp)t[(Rp/Op)n(Sp)m]y, or (Sp)t[(Rp/Op)n(Sp)m]y(Rp)k as described in the present disclosure.

In certain embodiments, a pattern of backbone chiral centers of a RNAi oligonucleotide is (Np)f(Op)g[(Rp/Op)n(Sp)m]y(Rp)k(Op)h(Np)j, (Np)f(Op)g[(Rp/Op)n(Sp)m]y(Op)h(Np)j, (Np)f(Op)g(Sp)t[(Rp/Op)n(Sp)m]y(Op)h(Np)j, or (Np)f(Op)g(Sp)t[(Rp/Op)n(Sp)m]y(Rp)k(Op)h(Np)j, and the oligonucleotide comprises a core region whose pattern of backbone chiral centers comprises or is [(Rp/Op)n(Sp)m]y(Rp)k, [(Rp/Op)n(Sp)m]y, (Sp)t[(Rp/Op)n(Sp)m]y, or (Sp)t[(Rp/Op)n(Sp)m]y(Rp)k as described in the present disclosure. In certain embodiments, a pattern of backbone chiral centers is or comprises (Np)f(Op)g[(Rp/Op)n(Sp)m]y(Rp)k(Op)h(Np)j.

In certain embodiments, a pattern of backbone chiral centers is or comprises (Np)f(Op)g[(Rp/Op)n(Sp)m]y(Rp)(Op)h(Np)j. In certain embodiments, a pattern of backbone chiral centers is or comprises (Np)f(Op)g[(Rp/Op)n(Sp)m]y(Op)h(Np)j. In certain embodiments, a pattern of backbone chiral centers is or comprises (Np)f(Op)g(Sp)t[(Rp/Op)n(Sp)m]y(Op)h(Np)j. In certain embodiments, a pattern of backbone chiral centers is or comprises (Np)f(Op)g(Sp)t[(Rp/Op)n(Sp)m]y(Rp)k(Op)h(Np)j. In certain embodiments, a pattern of backbone chiral centers is or comprises (Np)f(Op)g(Sp)t[(Rp/Op)n(Sp)m]y(Rp)(Op)h(Np)j.

In certain embodiments, a pattern of backbone chiral centers is or comprises (Np)f(Op)g[(Rp)n(Sp)m]y(Rp)k(Op)h(Np)j. In certain embodiments, a pattern of backbone chiral centers is or comprises (Np)f(Op)g[(Rp)n(Sp)m]y(Rp)(Op)h(Np)j. In certain embodiments, a pattern of backbone chiral centers is or comprises (Np)f(Op)g[(Rp)n(Sp)m]y(Op)h(Np)j. In certain embodiments, a pattern of backbone chiral centers is or comprises (Np)f(Op)g(Sp)t[(Rp)n(Sp)m]y(Op)h(Np)j.

In certain embodiments, a pattern of backbone chiral centers is or comprises (Np)f(Op)g(Sp)t[(Rp)n(Sp)m]y(Rp)k(Op)h(Np)j. In certain embodiments, a pattern of backbone chiral centers is or comprises (Np)f(Op)g(Sp)t[(Rp)n(Sp)m]y(Rp)(Op)h(Np)j.

In certain embodiments, at least one Np is Sp. In certain embodiments, at least one Np is Rp. In certain embodiments, the 5′ most Np is Sp. In certain embodiments, the 3′ most Np is Sp. In certain embodiments, each Np is Sp. In certain embodiments, (Np)f(Op)g[(Rp/Op)n(Sp)m]y(Rp)k(Op)h(Np)j is (Sp)(Op)g[(Rp)n(Sp)m]y(Rp)k(Op)h(Sp).

In certain embodiments, (Np)f(Op)g[(Rp/Op)n(Sp)m]y(Rp)k(Op)h(Np)j is (Sp)(Op)g[(Rp)n(Sp)m]y(Rp)(Op)h(Sp). In certain embodiments, a pattern of backbone chiral center of a ds oligonucleotide is or comprises (Sp)(Op)g[(Rp)n(Sp)m]y(Rp)(Op)h(Sp). In certain embodiments, a pattern of backbone chiral center of a ds oligonucleotide is (Sp)(Op)g[(Rp)n(Sp)m]y(Rp)(Op)h(Sp). In certain embodiments, (Np)f(Op)g[(Rp/Op)n(Sp)m]y(Op)h(Np)j is (Sp)(Op)g[(Rp)n(Sp)m]y(Op)h(Sp). In certain embodiments, a pattern of backbone chiral center of a ds oligonucleotide is or comprises (Sp)(Op)g[(Rp)n(Sp)m]y(Op)h(Sp). In certain embodiments, a pattern of backbone chiral center of a ds oligonucleotide is (Sp)(Op)g[(Rp)n(Sp)m]y(Op)h(Sp).

In certain embodiments, (Np)f(Op)g(Sp)t[(Rp/Op)n(Sp)m]y(Op)h(Np)j is (Sp)(Op)g(Sp)t[(Rp)n(Sp)m]y(Op)h(Sp). In certain embodiments, a pattern of backbone chiral center of a ds oligonucleotide is or comprises (Sp)(Op)g(Sp)t[(Rp)n(Sp)m]y(Op)h(Sp). In certain embodiments, a pattern of backbone chiral center of a ds oligonucleotide is (Sp)(Op)g(Sp)t[(Rp)n(Sp)m]y(Op)h(Sp). In certain embodiments, (Np)f(Op)g(Sp)t[(Rp/Op)n(Sp)m]y(Rp)k(Op)h(Np)j is (Sp)(Op)g(Sp)t[(Rp)n(Sp)m]y(Rp)k(Op)h(Sp).

In certain embodiments, (Np)f(Op)g(Sp)t[(Rp/Op)n(Sp)m]y(Rp)k(Op)h(Np)j is (Sp)(Op)g(Sp)t[(Rp)n(Sp)m]y(Rp)(Op)h(Sp). In certain embodiments, a pattern of backbone chiral center of a ds oligonucleotide is or comprises (Sp)(Op)g(Sp)t[(Rp)n(Sp)m]y(Rp)(Op)h(Sp). In certain embodiments, a pattern of backbone chiral center of a ds oligonucleotide is (Sp)(Op)g(Sp)t[(Rp)n(Sp)m]y(Rp)(Op)h(Sp). In certain embodiments, each n is 1. In certain embodiments, f is 1. In certain embodiments, g is 1. In certain embodiments, g is greater than 1. In certain embodiments, g is 2. In certain embodiments, g is 3. In certain embodiments, g is 4. In certain embodiments, g is 5. In certain embodiments, g is 6. In certain embodiments, g is 7. In certain embodiments, g is 8. In certain embodiments, g is 9. In certain embodiments, g is 10. In certain embodiments, h is 1. In certain embodiments, h is greater than 1. In certain embodiments, h is 2. In certain embodiments, h is 3. In certain embodiments, h is 4. In certain embodiments, h is 5. In certain embodiments, h is 6. In certain embodiments, h is 7. In certain embodiments, h is 8. In certain embodiments, h is 9. In certain embodiments, h is 10. In certain embodiments, j is 1. In certain embodiments, k is 1. In certain embodiments, k is 2-10.

In certain embodiments, a pattern of backbone chiral centers of a RNAi oligonucleotide or a region thereof (e.g., a core) comprises or is [(Rp/Op)n(Sp)m]y, (Sp)t[(Rp/Op)n(Sp)m]y, (Sp)t[(Rp/Op)n(Sp)m]yRp, [(Rp/Op)n(Sp)m]y(Rp)k, (Sp)t[(Rp/Op)n(Sp)m]y(Rp)k, (Sp)t[(Rp/Op)n(Sp)m]y(Rp)k(Op)h, (Sp)t[(Rp/Op)n(Sp)m]y(Rp)k(Op)h(Np)j, wherein each variable is independently as described in the present disclosure.

In certain embodiments, in a provided pattern of backbone chiral centers, at least one (Rp/Op) is Rp. In certain embodiments, at least one (Rp/Op) is Op. In certain embodiments, each (Rp/Op) is Rp. In certain embodiments, each (Rp/Op) is Op. In certain embodiments, at least one of [(Rp)n(Sp)m]y or [(Rp/Op)n(Sp)m]y of a pattern is RpSp. In certain embodiments, at least one of [(Rp)n(Sp)m]y or [(Rp/Op)n(Sp)m]y of a pattern is or comprises RpSpSp. In certain embodiments, at least one of [(Rp)n(Sp)m]y or [(Rp/Op)n(Sp)m]y in a pattern is RpSp, and at least one of [(Rp)n(Sp)m]y or [(Rp/Op)n(Sp)m]y in a pattern is or comprises RpSpSp. For example, in certain embodiments, [(Rp)n(Sp)m]y in a pattern is (RpSp)[(Rp)n(Sp)m]_{(y-1)}; in certain embodiments, [(Rp)n(Sp)m]y in a pattern is (RpSp)[RpSpSp(Sp)₍ₘ₋₂₎][(Rp)n(Sp)m]_{(y-2)}. In certain embodiments, (Sp)t[(Rp)n(Sp)m]y(Rp) is (Sp)t(RpSp)[(Rp)n(Sp)m]_{(y-1)}(Rp). In certain embodiments, (Sp)t[(Rp)n(Sp)m]y(Rp) is (Sp)t(RpSp)[RpSpSp(Sp)₍ₘ₋₂₎][(Rp)n(Sp)m]_{(y-2)}(Rp). In certain embodiments, each [(Rp/Op)n(Sp)m] is independently [Rp(Sp)m]. In certain embodiments, the first Sp of (Sp)t represents linkage phosphorus stereochemistry of the first internucleotidic linkage of a ds oligonucleotide from 5′ to 3′. In certain embodiments, the first Sp of (Sp)t represents linkage phosphorus stereochemistry of the first internucleotidic linkage of a region from 5′ to 3′, e.g., a core. In certain embodiments, the last Np of (Np)j represents linkage phosphorus stereochemistry of the last internucleotidic linkage of the oligonucleotide from 5′ to 3′. In certain embodiments, the last Np is Sp.

In certain embodiments, a pattern of backbone chiral centers of a ds oligonucleotide or a region (e.g., of a 5′-wing) is or comprises Sp(Op)₃. In certain embodiments, a pattern of backbone chiral centers of a ds oligonucleotide or a region (e.g., of a 5′-wing) is or comprises Rp(Op)₃. In certain embodiments, a pattern of backbone chiral centers of a ds oligonucleotide or a region (e.g., of a 3′-wing) is or comprises (Op)₃Sp. In certain embodiments, a pattern of backbone chiral centers of a ds oligonucleotide or a region (e.g., of a 3′-wing) is or comprises (Op)₃Rp. In certain embodiments, a pattern of backbone chiral centers of a ds oligonucleotide or a region (e.g., of a core) is or comprises Rp(Sp)₄Rp(Sp)₄Rp. In certain embodiments, a pattern of backbone chiral centers of a ds oligonucleotide or a region (e.g., of a core) is or comprises (Sp)₅Rp(Sp)₄Rp. In certain embodiments, a pattern of backbone chiral centers of a ds oligonucleotide or a region (e.g., of a core) is or comprises (Sp)₅Rp(Sp)₅. In certain embodiments, a pattern of backbone chiral centers of a ds oligonucleotide or a region (e.g., of a core) is or comprises Rp(Sp)₄Rp(Sp)₅. In certain embodiments, a pattern of backbone chiral centers of a ds oligonucleotide is or comprises Np(Op)₃Rp(Sp)₄Rp(Sp)₄Rp(Op)₃Np. In certain embodiments, a pattern of backbone chiral centers of a ds oligonucleotide is or comprises Np(Op)₃(Sp)₅Rp(Sp)₄Rp(Op)₃Np. In certain embodiments, a pattern of backbone chiral centers of a ds oligonucleotide is or comprises Np(Op)₃(Sp)₅Rp(Sp)₅(Op)₃Np. In certain embodiments, a pattern of backbone chiral centers of a ds oligonucleotide is or comprises Np(Op)₃Rp(Sp)₄Rp(Sp)₅(Op)₃Np. In certain embodiments, a pattern of backbone chiral centers of a ds oligonucleotide is or comprises Sp(Op)₃Rp(Sp)₄Rp(Sp)₄Rp(Op)₃Sp. In certain embodiments, a pattern of backbone chiral centers of a ds oligonucleotide is or comprises Sp(Op)₃(Sp)₅Rp(Sp)₄Rp(Op)₃Sp. In certain embodiments, a pattern of backbone chiral centers of a ds oligonucleotide is or comprises Sp(Op)₃(Sp)₅Rp(Sp)₅(Op)₃Sp. In certain embodiments, a pattern of backbone chiral centers of a ds oligonucleotide is or comprises Sp(Op)₃Rp(Sp)₄Rp(Sp)₅(Op)₃Sp. In certain embodiments, a pattern of backbone chiral centers of a ds oligonucleotide is or comprises Rp(Op)₃Rp(Sp)₄Rp(Sp)₄Rp(Op)₃Rp. In certain embodiments, a pattern of backbone chiral centers of a ds oligonucleotide is or comprises Rp(Op)₃(Sp)₅Rp(Sp)₄Rp(Op)₃Rp. In certain embodiments, a pattern of backbone chiral centers of a ds oligonucleotide is or comprises Rp(Op)₃(Sp)₅Rp(Sp)₅(Op)₃Rp. In certain embodiments, a pattern of backbone chiral centers of a ds oligonucleotide is or comprises Rp(Op)₃Rp(Sp)₄Rp(Sp)₅(Op)₃Rp.

In certain embodiments, each of m, y, t, n, k, f, g, h, and j is independently 1-25.

In certain embodiments, m is 1-25. In certain embodiments, m is 1-20. In certain embodiments, m is 1-15. In certain embodiments, m is 1-10. In certain embodiments, m is 1-5. In certain embodiments, m is 2-20. In certain embodiments, m is 2-15. In certain embodiments, m is 2-10. In certain embodiments, m is 2-5. In certain embodiments, m is 1, 2, 3, 4, 5, 6, 7, 8, 9, 10, 11, 12, 13, 14, 15, 16, 17, 18, 19, 20, 21, 22, 23, 24, or 25. In certain embodiments, in a pattern of backbone chiral centers each m is independently 2 or more. In certain embodiments, each m is independently 2, 3, 4, 5, 6, 7, 8, 9, or 10. In certain embodiments, each m is independently 2-3, 2-5, 2-6, or 2-10. In certain embodiments, m is 2. In certain embodiments, m is 3. In certain embodiments, m is 4. In certain embodiments, m is 5. In certain embodiments, m is 6. In certain embodiments, m is 7. In certain embodiments, m is 8. In certain embodiments, m is 9. In certain embodiments, m is 10. In certain embodiments, where there are two or more occurrences of m, they can be the same or different, and each of them is independently as described in the present disclosure.

In certain embodiments, y is 1-25. In certain embodiments, y is 1-20. In certain embodiments, y is 1-15. In certain embodiments, y is 1-10. In certain embodiments, y is 1-5. In certain embodiments, y is 2-20. In certain embodiments, y is 2-15. In certain embodiments, y is 2-10. In certain embodiments, y is 2-5. In certain embodiments, y is 1, 2, 3, 4, 5, 6, 7, 8, 9, 10, 11, 12, 13, 14, 15, 16, 17, 18, 19, 20, 21, 22, 23, 24, or 25. In certain embodiments, y is 1, 2, 3, 4, 5, 6, 7, 8, 9, or 10. In certain embodiments, y is 1. In certain embodiments, y is 2. In certain embodiments, y is 3. In certain embodiments, y is 4. In certain embodiments, y is 5. In certain embodiments, y is 6. In certain embodiments, y is 7. In certain embodiments, y is 8. In certain embodiments, y is 9. In certain embodiments, y is 10.

In certain embodiments, t is 1-25. In certain embodiments, t is 1-20. In certain embodiments, t is 1-15. In certain embodiments, t is 1-10. In certain embodiments, t is 1-5. In certain embodiments, t is 2-20. In certain embodiments, t is 2-15. In certain embodiments, t is 2-10. In certain embodiments, t is 2-5. In certain embodiments, t is 1, 2, 3, 4, 5, 6, 7, 8, 9, 10, 11, 12, 13, 14, 15, 16, 17, 18, 19, 20, 21, 22, 23, 24, or 25. In certain embodiments, each t is independently 1, 2, 3, 4, 5, 6, 7, 8, 9, or 10. In certain embodiments, t is 2 or more. In certain embodiments, t is 1. In certain embodiments, t is 2. In certain embodiments, t is 3. In certain embodiments, t is 4. In certain embodiments, t is 5. In certain embodiments, t is 6. In certain embodiments, t is 7. In certain embodiments, t is 8. In certain embodiments, t is 9. In certain embodiments, t is 10. In certain embodiments, where there are two or more occurrences of t, they can be the same or different, and each of them is independently as described in the present disclosure.

In certain embodiments, n is 1-25. In certain embodiments, n is 1, 2, 3, 4, 5, 6, 7, 8, 9, 10, 11, 12, 13, 14, 15, 16, 17, 18, 19, 20, 21, 22, 23, 24, or 25. In certain embodiments, n is 1. In certain embodiments, n is 2. In certain embodiments, n is 3. In certain embodiments, n is 4. In certain embodiments, n is 5. In certain embodiments, n is 6. In certain embodiments, n is 7. In certain embodiments, n is 8. In certain embodiments, n is 9. In certain embodiments, n is 10. In certain embodiments, where there are two or more occurrences of n, they can be the same or different, and each of them is independently as described in the present disclosure. In certain embodiments, in a pattern of backbone chiral centers, at least one occurrence of n is 1; in some cases, each n is 1.

In certain embodiments, k is 1-25. In certain embodiments, k is 1, 2, 3, 4, 5, 6, 7, 8, 9, 10, 11, 12, 13, 14, 15, 16, 17, 18, 19, 20, 21, 22, 23, 24, or 25. In certain embodiments, k is 1. In certain embodiments, k is 2. In certain embodiments, k is 3. In certain embodiments, k is 4. In certain embodiments, k is 5. In certain embodiments, k is 6. In certain embodiments, k is 7. In certain embodiments, k is 8. In certain embodiments, k is 9. In certain embodiments, k is 10.

In certain embodiments, f is 1-25. In certain embodiments, f is 1-20. In certain embodiments, f is 1-10. In certain embodiments, f is 1-5. In certain embodiments, f is 1, 2, 3, 4, 5, 6, 7, 8, 9, 10, 11, 12, 13, 14, 15, 16, 17, 18, 19, 20, 21, 22, 23, 24, or 25. In certain embodiments, f is 1. In certain embodiments, f is 2. In certain embodiments, f is 3. In certain embodiments, f is 4. In certain embodiments, f is 5. In certain embodiments, f is 6. In certain embodiments, f is 7. In certain embodiments, f is 8. In certain embodiments, f is 9. In certain embodiments, f is 10.

In certain embodiments, g is 1-25. In certain embodiments, g is 1-20. In certain embodiments, g is 1-9. In certain embodiments, g is 1-5. In certain embodiments, g is 2-10. In certain embodiments, g is 2-5. In certain embodiments, g is 1, 2, 3, 4, 5, 6, 7, 8, 9, 10, 11, 12, 13, 14, 15, 16, 17, 18, 19, 20, 21, 22, 23, 24, or 25. In certain embodiments, g is 1. In certain embodiments, g is 2. In certain embodiments, g is 3. In certain embodiments, g is 4. In certain embodiments, g is 5. In certain embodiments, g is 6. In certain embodiments, g is 7. In certain embodiments, g is 8. In certain embodiments, g is 9. In certain embodiments, g is 10.

In certain embodiments, h is 1-25. In certain embodiments, h is 1-10. In certain embodiments, h is 1-5. In certain embodiments, h is 2-10. In certain embodiments, h is 2-5. In certain embodiments, h is 1, 2, 3, 4, 5, 6, 7, 8, 9, 10, 11, 12, 13, 14, 15, 16, 17, 18, 19, 20, 21, 22, 23, 24, or 25. In certain embodiments, h is 1. In certain embodiments, h is 2. In certain embodiments, h is 3. In certain embodiments, h is 4. In certain embodiments, h is 5. In certain embodiments, h is 6. In certain embodiments, h is 7. In certain embodiments, h is 8. In certain embodiments, h is 9. In certain embodiments, h is 10.

In certain embodiments, j is 1-25. In certain embodiments, j is 1-10. In certain embodiments, j is 1-5. In certain embodiments, j is 1, 2, 3, 4, 5, 6, 7, 8, 9, 10, 11, 12, 13, 14, 15, 16, 17, 18, 19, 20, 21, 22, 23, 24, or 25. In certain embodiments, j is 1. In certain embodiments, j is 2. In certain embodiments, j is 3. In certain embodiments, j is 4. In certain embodiments, j is 5. In certain embodiments, j is 6. In certain embodiments, j is 7. In certain embodiments, j is 8. In certain embodiments, j is 9. In certain embodiments, j is 10.

In certain embodiments, at least one n is 1, and at least one m is no less than 2. In certain embodiments, at least one n is 1, at least one t is no less than 2, and at least one m is no less than 3. In certain embodiments, each n is 1. In certain embodiments, t is 1. In certain embodiments, at least one t>1. In certain embodiments, at least one t>2. In certain embodiments, at least one t>3. In certain embodiments, at least one t>4. In certain embodiments, at least one m>1. In certain embodiments, at least one m>2. In certain embodiments, at least one m>3. In certain embodiments, at least one m>4. In certain embodiments, a pattern of backbone chiral centers comprises one or more achiral natural phosphate linkages. In certain embodiments, the sum of m, t, and n (or m and n if no t is in a pattern) is no less than 5, 6, 7, 8, 9, 10, 11, 12, 13, 14, 15, 16, 17, 18, 19 or 20. In certain embodiments, the sum is 5. In certain embodiments, the sum is 6. In certain embodiments, the sum is 7. In certain embodiments, the sum is 8. In certain embodiments, the sum is 9. In certain embodiments, the sum is 10. In certain embodiments, the sum is 11. In certain embodiments, the sum is 12. In certain embodiments, the sum is 13. In certain embodiments, the sum is 14. In certain embodiments, the sum is 15.

In certain embodiments, a number of linkage phosphorus in chirally controlled internucleotidic linkages are Sp. In certain embodiments, at least 10%, 20%, 25%, 30%, 35%, 40%, 45%, 50%, 55%, 60%, 65%, 70%, 75%, 80%, 85%, 90% or 95% of chirally controlled internucleotidic linkages have Sp linkage phosphorus. In certain embodiments, at least 10%, 20%, 25%, 30%, 35%, 40%, 45%, 50%, 55%, 60%, 65%, 70%, 75%, 80%, 85%, 90% or 95% of all chiral internucleotidic linkages are chirally controlled internucleotidic linkages having Sp linkage phosphorus. In certain embodiments, at least 10%, 20%, 25%, 30%, 35%, 40%, 45%, 50%, 55%, 60%, 65%, 70%, 75%, 80%, 85%, 90% or 95% of all internucleotidic linkages are chirally controlled internucleotidic linkages having Sp linkage phosphorus. In certain embodiments, the percentage is at least 20%. In certain embodiments, the percentage is at least 30%. In certain embodiments, the percentage is at least 40%. In certain embodiments, the percentage is at least 50%. In certain embodiments, the percentage is at least 60%.

In certain embodiments, the percentage is at least 65%. In certain embodiments, the percentage is at least 70%. In certain embodiments, the percentage is at least 75%. In certain embodiments, the percentage is at least 80%. In certain embodiments, the percentage is at least 90%. In certain embodiments, the percentage is at least 95%. In certain embodiments, at least 1, 2, 3, 4, 5, 6, 7, 8, 9, 10, 11, 12, 13, 14, 15, 16, 17, 18, 19, 20, 21, 22, 23, 24, or 25 internucleotidic linkages are chirally controlled internucleotidic linkages having Sp linkage phosphorus. In certain embodiments, at least 5 internucleotidic linkages are chirally controlled internucleotidic linkages having Sp linkage phosphorus. In certain embodiments, at least 6 internucleotidic linkages are chirally controlled internucleotidic linkages having Sp linkage phosphorus. In certain embodiments, at least 7 internucleotidic linkages are chirally controlled internucleotidic linkages having Sp linkage phosphorus. In certain embodiments, at least 8 internucleotidic linkages are chirally controlled internucleotidic linkages having Sp linkage phosphorus. In certain embodiments, at least 9 internucleotidic linkages are chirally controlled internucleotidic linkages having Sp linkage phosphorus. In certain embodiments, at least 10 internucleotidic linkages are chirally controlled internucleotidic linkages having Sp linkage phosphorus. In certain embodiments, at least 11 internucleotidic linkages are chirally controlled internucleotidic linkages having Sp linkage phosphorus. In certain embodiments, at least 12 internucleotidic linkages are chirally controlled internucleotidic linkages having Sp linkage phosphorus. In certain embodiments, at least 13 internucleotidic linkages are chirally controlled internucleotidic linkages having Sp linkage phosphorus. In certain embodiments, at least 14 internucleotidic linkages are chirally controlled internucleotidic linkages having Sp linkage phosphorus. In certain embodiments, at least 15 internucleotidic linkages are chirally controlled internucleotidic linkages having Sp linkage phosphorus. In certain embodiments, at least 1, 2, 3, 4, 5, 6, 7, 8, 9, 10, 11, 12, 13, 14, 15, 16, 17, 18, 19, 20, 21, 22, 23, 24, or 25 internucleotidic linkages are chirally controlled internucleotidic linkages having Rp linkage phosphorus. In certain embodiments, no more than 1, 2, 3, 4, 5, 6, 7, 8, 9, 10, 11, 12, 13, 14, 15, 16, 17, 18, 19, 20, 21, 22, 23, 24, or 25 internucleotidic linkages are chirally controlled internucleotidic linkages having Rp linkage phosphorus. In certain embodiments, one and no more than one internucleotidic linkage in a ds oligonucleotide is a chirally controlled internucleotidic linkage having Rp linkage phosphorus. In certain embodiments, 2 and no more than 2 internucleotidic linkages in a ds oligonucleotide are chirally controlled internucleotidic linkages having Rp linkage phosphorus. In certain embodiments, 3 and no more than 3 internucleotidic linkages in a ds oligonucleotide are chirally controlled internucleotidic linkages having Rp linkage phosphorus. In certain embodiments, 4 and no more than 4 internucleotidic linkages in a ds oligonucleotide are chirally controlled internucleotidic linkages having Rp linkage phosphorus. In certain embodiments, 5 and no more than 5 internucleotidic linkages in a ds oligonucleotide are chirally controlled internucleotidic linkages having Rp linkage phosphorus.

In certain embodiments, all, essentially all or most of the internucleotidic linkages in a ds oligonucleotide are in the Sp configuration (e.g., about 50%-100%, 55%-100%, 60%-100%, 65%-100%, 70%-100%, 75%-100%, 80%-100%, 85%-100%, 90%-100%, 55%-95%, 60%-95%, 65%-95%, or about 55%, 60%, 65%, 70%, 75%, 80%, 85%, 90%, 95%, 97%, 99% r more of all chirally controlled internucleotidic linkages, or of all chiral internucleotidic linkages, or of all internucleotidic linkages in the oligonucleotide) except for one or a minority of internucleotidic linkages (e.g., 1, 2, 3, 4, or 5, and/or less than 50%, 45%, 40%, 35%, 30%, 25%, 20%, 15%, 10%, or 5% f all chirally controlled internucleotidic linkages, or of all chiral internucleotidic linkages, or of all internucleotidic linkages in the oligonucleotide) being in the Rp configuration. In certain embodiments, all, essentially all or most of the internucleotidic linkages in a core are in the Sp configuration (e.g., about 50%-100%, 55%-100%, 60%-100%, 65%-100%, 70%-100%, 75%-100%, 80%-100%, 85%-100%, 90%-100%, 55%-95%, 60%-95%, 65%-95%, or about 55%, 60%, 65%, 70%, 75%, 80%, 85%, 90%, 95%, 97%, 99% r more of all chirally controlled internucleotidic linkages, or of all chiral internucleotidic linkages, or of all internucleotidic linkages, in the core) except for one or a minority of internucleotidic linkages (e.g., 1, 2, 3, 4, or 5, and/or less than 50%, 45%, 40%, 35%, 30%, 25%, 20%, 15%, 10%, or 5% f all chirally controlled internucleotidic linkages, or of all chiral internucleotidic linkages, or of all internucleotidic linkages, in the core) being in the Rp configuration. In certain embodiments, all, essentially all or most of the internucleotidic linkages in the core are a phosphoryl guanidine or a phosphorothioate in the Sp configuration (e.g., about 50%-100%, 55%-100%, 60%-100%, 65%-100%, 70%-100%, 75%-100%, 80%-100%, 85%-100%, 90%-100%, 55%-95%, 60%-95%, 65%-95%, or about 55%, 60%, 65%, 70%, 75%, 80%, 85%, 90%, 95%, 97%, 99% r more of all chirally controlled internucleotidic linkages, or of all chiral internucleotidic linkages, or of all internucleotidic linkages, in the core) except for one or a minority of internucleotidic linkages (e.g., 1, 2, 3, 4, or 5, and/or less than 50%, 45%, 40%, 35%, 30%, 25%, 20%, 15%, 10%, or 5% f all chirally controlled internucleotidic linkages, or of all chiral internucleotidic linkages, or of all internucleotidic linkages, in the core) being a phosphorothioate in the Rp configuration. In certain embodiments, each interucleotidic linkage in the core is a phosphoryl guanidine in the Sp configuration. In certain embodiments, each internucleotidic linkage in the core is a phosphorothioate in the Sp configuration except for one phosphorothioate in the Rp configuration. In certain embodiments, each internucleotidic linkage in the core is a phosphorothioate in the Sp configuration except for one phosphorothioate in the Rp configuration.

In certain embodiments, a ds oligonucleotide comprises one or more Rp internucleotidic linkages. In certain embodiments, a ds oligonucleotide comprises one and no more than one Rp internucleotidic linkages. In certain embodiments, a ds oligonucleotide comprises two or more Rp internucleotidic linkages. In certain embodiments, a ds oligonucleotide comprises three or more Rp internucleotidic linkages. In certain embodiments, a ds oligonucleotide comprises four or more Rp internucleotidic linkages. In certain embodiments, a ds oligonucleotide comprises five or more Rp internucleotidic linkages. In certain embodiments, about 5%-50% f all chirally controlled internucleotidic linkages in a ds oligonucleotide are Rp. In certain embodiments, about 5%-40% f all chirally controlled internucleotidic linkages in a ds oligonucleotide are Rp. In certain embodiments, about 10%-40% f all chirally controlled internucleotidic linkages in a ds oligonucleotide are Rp. In certain embodiments, about 15%-40% f all chirally controlled internucleotidic linkages in a ds oligonucleotide are Rp. In certain embodiments, about 20%-40% f all chirally controlled internucleotidic linkages in a ds oligonucleotide are Rp. In certain embodiments, about 25%-40% f all chirally controlled internucleotidic linkages in a ds oligonucleotide are Rp. In certain embodiments, about 30%-40% f all chirally controlled internucleotidic linkages in a ds oligonucleotide are Rp. In certain embodiments, about 35%-40% f all chirally controlled internucleotidic linkages in a ds oligonucleotide are Rp.

In certain embodiments, instead of an Rp internucleotidic linkage, a natural phosphate linkage may be similarly utilized, optionally with a modification, e.g., a sugar modification (e.g., a 5′-modification such as R^{5s} as described herein). In certain embodiments, a modification improves stability of a natural phosphate linkage.

In certain embodiments, the present disclosure provides a ds oligonucleotide having a pattern of backbone chiral centers as described herein. In certain embodiments, oligonucleotides in a chirally controlled ds oligonucleotide composition share a common pattern of backbone chiral centers as described herein.

In certain embodiments, at least about 25% f the internucleotidic linkages of a dsRNAi oligonucleotide are chirally controlled and have Sp linkage phosphorus. In certain embodiments, at least about 30% f the internucleotidic linkages of a ds oligonucleotide are chirally controlled and have Sp linkage phosphorus. In certain embodiments, at least about 40% f the internucleotidic linkages of a provided ds oligonucleotide are chirally controlled and have Sp linkage phosphorus. In certain embodiments, at least about 50% f the internucleotidic linkages of a provided ds oligonucleotide are chirally controlled and have Sp linkage phosphorus. In certain embodiments, at least about 60% f the internucleotidic linkages of a provided ds oligonucleotide are chirally controlled and have Sp linkage phosphorus. In certain embodiments, at least about 65% f the internucleotidic linkages of a provided ds oligonucleotide are chirally controlled and have Sp linkage phosphorus. In certain embodiments, at least about 70% f the internucleotidic linkages of a provided ds oligonucleotide are chirally controlled and have Sp linkage phosphorus. In certain embodiments, at least about 75% f the internucleotidic linkages of a provided ds oligonucleotide are chirally controlled and have Sp linkage phosphorus. In certain embodiments, at least about 80% f the internucleotidic linkages of a provided ds oligonucleotide are chirally controlled and have Sp linkage phosphorus. In certain embodiments, at least about 85% f the internucleotidic linkages of a provided ds oligonucleotide are chirally controlled and have Sp linkage phosphorus. In certain embodiments, at least about 90% f the internucleotidic linkages of a provided ds oligonucleotide are chirally controlled and have Sp linkage phosphorus. In certain embodiments, at least about 95% f the internucleotidic linkages of a provided ds oligonucleotide are chirally controlled and have Sp linkage phosphorus.

In certain embodiments, the present disclosure provides chirally controlled ds oligonucleotide compositions, e.g., chirally controlled dsRNAi oligonucleotide compositions, wherein the composition comprises a non-random or controlled level of a plurality of oligonucleotides, wherein oligonucleotides of the plurality share a common base sequence, and share the same configuration of linkage phosphorus independently at 1-50, 1-40, 1-30, 1-25, 1-20, 1-15, 1-10, 5-50, 5-40, 5-30, 5-25, 5-20, 5-15, 5-10, 1, 2, 3, 4, 5, 6, 7, 8, 9, 10, 11, 12, 13, 14, 15, 16, 17, 18, 19, 20, 21, 22, 23, 24, or 25 or more chiral internucleotidic linkages.

In certain embodiments, dsRNAi oligonucleotides comprise 2-30 chirally controlled internucleotidic linkages. In certain embodiments, provided ds oligonucleotide compositions comprise 5-30 chirally controlled internucleotidic linkages. In certain embodiments, provided ds oligonucleotide compositions comprise 10-30 chirally controlled internucleotidic linkages.

In certain embodiments, a percentage is about 5%-100%. In certain embodiments, a percentage is at least 50%, 55%, 60%, 65%, 70%, 75%, 80%, 85%, 90%, 95%, 965, 96%, 98%, or 99%. In certain embodiments, a percentage is about 5%, 10%, 15%, 20%, 25%, 30%, 35%, 40%, 45%, 50%, 55%, 60%, 65%, 70%, 75%, 80%, 85%, 90%, 95%, 965, 96%, 98%, or 99%.

In certain embodiments, a pattern of backbone chiral centers in a dsRNAi oligonucleotide comprises a pattern of i^{o}-i^{s}-i^{o}-i^{s}-i^{o}, i^{o}-i^{s}-i^{s}-i^{s}-i^{o}, i^{o}-i^{s}-i^{s}-i^{s}-i^{o}-i^{s}, i^{s}-i^{o}-i^{s}-i^{o}, i^{s}-i^{o}-i^{s}-i^{o}, i^{s}-i^{o}-i^{s}-i^{o}-i^{s}, i^{s}-i^{o}-i^{s}-i^{o}-i^{s}-i^{o}, i^{s}-i^{o}-i^{s}-i^{o}-i^{s}-i^{o}-i^{s}-i^{o}, i^{s}-i^{o}-i^{s}-i^{s}-i^{s}-i^{o}, i^{s}-i^{s}-i^{o}-i^{s}-i^{s}-i^{s}-i^{o}-i^{s}-i^{s}, i^{s}-i^{s}-i^{s}-i^{o}-i^{s}-i^{o}-i^{s}-i^{s}-i^{s}, i^{s}-i^{s}-i^{s}-i^{s}-i^{o}-i^{s}-i^{o}-i^{s}-i^{s}-i^{s}-i^{s}, i^{s}-i^{s}-i^{s}-i^{s}-i^{s}, i^{s}-i^{s}-i^{s}-i^{s}-i^{s}-i^{s}, i^{s}-i^{s}-i^{s}-i^{s}-i^{s}-i^{s}-i^{s}, i^{s}-i^{s}-i^{s}-i^{s}-i^{s}-i^{s}-i^{s}-i^{s}, i^{s}-i^{s}-i^{s}-i^{s}-i^{s}-i^{s}-i^{s}-i^{s}-i^{s}, or i^{r}-i^{r}-i^{r}, wherein i^{s} represents an internucleotidic linkage in the Sp configuration; i^{o} represents an achiral internucleotidic linkage; and i^{r} represents an internucleotidic linkage in the Rp configuration.

In certain embodiments, an internucleotidic linkage in the Sp configuration (having a Sp linkage phosphorus) is a phosphoryl guanidine internucleotidic linkage. In certain embodiments, an achiral internucleotidic linkage is a natural phosphate linkage. In certain embodiments, an internucleotidic linkage in the Rp configuration (having a Rp linkage phosphorus) is a phosphorothioate internucleotidic linkage. In certain embodiments, each internucleotidic linkage in the Sp configuration is a phosphoryl guanidine internucleotidic linkage. In certain embodiments, each achiral internucleotidic linkage is a natural phosphate linkage. In certain embodiments, each internucleotidic linkage in the Rp configuration is a phosphoryl guanidine internucleotidic linkage. In certain embodiments, each internucleotidic linkage in the Sp configuration is a phosphorothioate internucleotidic linkage, each achiral internucleotidic linkage is a natural phosphate linkage, and each internucleotidic linkage in the Rp configuration is a phosphoryl guanidine internucleotidic linkage. In certain embodiments, an internucleotidic linkage in the Sp configuration (having a Sp linkage phosphorus) is a phosphorothioate internucleotidic linkage. In certain embodiments, an achiral internucleotidic linkage is a natural phosphate linkage. In certain embodiments, an internucleotidic linkage in the Rp configuration (having a Rp linkage phosphorus) is a phosphorothioate internucleotidic linkage. In certain embodiments, each internucleotidic linkage in the Sp configuration is a phosphorothioate internucleotidic linkage. In certain embodiments, each achiral internucleotidic linkage is a natural phosphate linkage. In certain embodiments, each internucleotidic linkage in the Rp configuration is a phosphorothioate internucleotidic linkage. In certain embodiments, each internucleotidic linkage in the Sp configuration is a phosphorothioate internucleotidic linkage, each achiral internucleotidic linkage is a natural phosphate linkage, and each internucleotidic linkage in the Rp configuration is a phosphorothioate internucleotidic linkage.

In certain embodiments, dsRNAi oligonucleotides in chirally controlled oligonucleotide compositions each comprise different types of internucleotidic linkages. In certain embodiments, dsRNAi oligonucleotides comprise at least one natural phosphate linkage and at least one modified internucleotidic linkage. In certain embodiments, dsRNAi oligonucleotides comprise at least one natural phosphate linkage and at least two modified internucleotidic linkages. In certain embodiments, dsRNAi oligonucleotides comprise at least one natural phosphate linkage and at least three modified internucleotidic linkages. In certain embodiments, dsRNAi oligonucleotides comprise at least one natural phosphate linkage and at least four modified internucleotidic linkages. In certain embodiments, dsRNAi oligonucleotides comprise at least one natural phosphate linkage and at least five modified internucleotidic linkages. In certain embodiments, dsRNAi oligonucleotides comprise at least one natural phosphate linkage and 1, 2, 3, 4, 5, 6, 7, 8, 9, 10, 11, 12, 13, 14, 15, 16, 17, 18, 19, 20, 21, 22, 23, 24, or 25 modified internucleotidic linkages. In certain embodiments, a modified internucleotidic linkage is a phosphorothioate or a phosphoryl guanidine internucleotidic linkage. In certain embodiments, each modified internucleotidic linkage is a phosphorothioate or a phosphoryl guanidine internucleotidic linkage. In certain embodiments, a modified internucleotidic linkage is a phosphorothioate triester internucleotidic linkage. In certain embodiments, each modified interucleotidic linkage is a phosphorothioate or a phosphoryl guanidine triester internucleotidic linkage. In certain embodiments, RNAi oligonucleotides comprise at least one natural phosphate linkage and at least 2, 3, 4, 5, 6, 7, 8, 9, 10, 11, 12, 13, 14, 15, 16, 17, 18, 19, 20, 21, 22, 23, 24, or 25 consecutive modified internucleotidic linkages. In certain embodiments, RNAi oligonucleotides comprise at least one natural phosphate linkage and at least 2, 3, 4, 5, 6, 7, 8, 9, 10, 11, 12, 13, 14, 15, 16, 17, 18, 19, 20, 21, 22, 23, 24, or 25 consecutive phosphorothioate or a phosphoryl guanidine internucleotidic linkages. In certain embodiments, dsRNAi oligonucleotides comprise at least one natural phosphate linkage and at least 2, 3, 4, 5, 6, 7, 8, 9, 10, 11, 12, 13, 14, 15, 16, 17, 18, 19, 20, 21, 22, 23, 24, or 25 consecutive phosphorothioate or a phosphoryl guanidine triester internucleotidic linkages.

In certain embodiments, oligonucleotides in a chirally controlled ds oligonucleotide composition each comprise at least two internucleotidic linkages that have different stereochemistry and/or different P-modifications relative to one another. In certain embodiments, at least two internucleotidic linkages have different stereochemistry relative to one another, and the ds oligonucleotides each comprise a pattern of backbone chiral centers comprising alternating linkage phosphorus stereochemistry.

In certain embodiments, a linkage comprises a chiral auxiliary, which, for example, is used to control the stereoselectivity of a reaction, e.g., a coupling reaction in a ds oligonucleotide synthesis cycle. In certain embodiments, a phosphorothioate or a phosphoryl guanidine triester linkage does not comprise a chiral auxiliary. In certain embodiments, a phosphorothioate or a phosphoryl guanidine triester linkage is intentionally maintained until and/or during the administration of the oligonucleotide composition to a subject.

In certain embodiments, purity, particularly stereochemical purity, and particularly diastereomeric purity of many ds oligonucleotides and compositions thereof wherein all other chiral centers in the ds oligonucleotides but the chiral linkage phosphorus centers have been stereodefined (e.g., carbon chiral centers in the sugars, which are defined in, e.g., phosphoramidites for ds oligonucleotide synthesis), can be controlled by stereoselectivity (as appreciated by those skilled in this art, diastereoselectivity in many cases of ds oligonucleotide synthesis wherein the ds oligonucleotide comprise more than one chiral centers) at chiral linkage phosphorus in coupling steps when forming chiral internucleotidic linkages. In certain embodiments, a coupling step has a stereoselectivity (diastereoselectivity when there are other chiral centers) of 60% at the linkage phosphorus. After such a coupling step, the new internucleotidic linkage formed may be referred to have a 60% stereochemical purity (for ds oligonucleotides, typically diastereomeric purity in view of the existence of other chiral centers). In certain embodiments, each coupling step independently has a stereoselectivity of at least 60%. In certain embodiments, each coupling step independently has a stereoselectivity of at least 70%. In certain embodiments, each coupling step independently has a stereoselectivity of at least 80%. In certain embodiments, each coupling step independently has a stereoselectivity of at least 85%. In certain embodiments, each coupling step independently has a stereoselectivity of at least 90%. In certain embodiments, each coupling step independently has a stereoselectivity of at least 91%. In certain embodiments, each coupling step independently has a stereoselectivity of at least 92%. In certain embodiments, each coupling step independently has a stereoselectivity of at least 93%. In certain embodiments, each coupling step independently has a stereoselectivity of at least 94%. In certain embodiments, each coupling step independently has a stereoselectivity of at least 95%. In certain embodiments, each coupling step independently has a stereoselectivity of at least 96%. In certain embodiments, each coupling step independently has a stereoselectivity of at least 97%. In certain embodiments, each coupling step independently has a stereoselectivity of at least 98%. In certain embodiments, each coupling step independently has a stereoselectivity of at least 99%. In certain embodiments, each coupling step independently has a stereoselectivity of at least 99.5%. In certain embodiments, each coupling step independently has a stereoselectivity of virtually 100%. In certain embodiments, a coupling step has a stereoselectivity of virtually 100% in that each detectable product from the coupling step analyzed by an analytical method (e.g., NMR, HPLC, etc.) has the intended stereoselectivity. In certain embodiments, a chirally controlled internucleotidic linkage is typically formed with a stereoselectivity of at least 90%, 91%, 92%, 93%, 94%, 95%, 96%, 97%, 98%, 99.5% r virtually 100% (in certain embodiments, at least 90%; in certain embodiments, at least 95%; in certain embodiments, at least 96%; in certain embodiments, at least 97%; in certain embodiments, at least 98%; in certain embodiments, at least 99%). In certain embodiments, a chirally controlled internucleotidic linkage has a stereochemical purity (typically diastereomeric purity for oligonucleotides with multiple chiral centers) of at least 90%, 91%, 92%, 93%, 94%, 95%, 96%, 97%, 98%, 99.5% r virtually 100% (in certain embodiments, at least 90%; in certain embodiments, at least 95%; in certain embodiments, at least 96%; in certain embodiments, at least 97%; in certain embodiments, at least 98%; in certain embodiments, at least 99%) at its chiral linkage phosphorus. In certain embodiments, each chirally controlled internucleotidic linkage independently has a stereochemical purity (typically diastereomeric purity for oligonucleotides with multiple chiral centers) of at least 90%, 91%, 92%, 93%, 94%, 95%, 96%, 97%, 98%, 99.5% r virtually 100% (in certain embodiments, at least 90%; in certain embodiments, at least 95%; in certain embodiments, at least 96%; in certain embodiments, at least 97%; in certain embodiments, at least 98%; in certain embodiments, at least 99%) at its chiral linkage phosphorus. In certain embodiments, a non-chirally controlled internucleotidic linkage is typically formed with a stereoselectivity of less than 60%, 70%, 80%, 85%, or 90% (in certain embodiments, less than 60%; in certain embodiments, less than 70%; in certain embodiments, less than 80%; in certain embodiments, less than 85%; in certain embodiments, less than 90%). In certain embodiments, each non-chirally controlled internucleotidic linkage is independently formed with a stereoselectivity of less than 60%, 70%, 80%, 85%, or 90% (in certain embodiments, less than 60%; in certain embodiments, less than 70%; in certain embodiments, less than 80%; in certain embodiments, less than 85%; in certain embodiments, less than 90%). In certain embodiments, a non-chirally controlled internucleotidic linkage has a stereochemical purity (typically diastereomeric purity for oligonucleotides with multiple chiral centers) of less than 60%, 70%, 80%, 85%, or 90% (in certain embodiments, less than 60%; in certain embodiments, less than 70%; in certain embodiments, less than 80%; in certain embodiments, less than 85%; in certain embodiments, less than 90%) at its chiral linkage phosphorus. In certain embodiments, each non-chirally controlled internucleotidic linkage independently has a stereochemical purity (typically diastereomeric purity for oligonucleotides with multiple chiral centers) of less than 60%, 70%, 80%, 85%, or 90% (in certain embodiments, less than 60%; in certain embodiments, less than 70%; in certain embodiments, less than 80%; in certain embodiments, less than 85%; in certain embodiments, less than 90%) at its chiral linkage phosphorus.

In certain embodiments, at least 1, 2, 3, 4, 5, 6, 7, 8, 9, or 10 couplings of a monomer (as appreciated by those skilled in the art in certain embodiments a phosphoramidite for oligonucleotide synthesis) independently have a stereoselectivity less than about 60%, 70%, 80%, 85%, or 90% [for oligonucleotide synthesis, typically diastereoselectivity with respect to formed linkage phosphorus chiral center(s)]. In certain embodiments, at least one coupling has a stereoselectivity less than about 60%, 70%, 80%, 85%, or 90%. In certain embodiments, at least two couplings independently have a stereoselectivity less than about 60%, 70%, 80%, 85%, or 90%. In certain embodiments, at least three couplings independently have a stereoselectivity less than about 60%, 70%, 80%, 85%, or 90%. In certain embodiments, at least four couplings independently have a stereoselectivity less than about 60%, 70%, 80%, 85%, or 90%. In certain embodiments, at least five couplings independently have a stereoselectivity less than about 60%, 70%, 80%, 85%, or 90%. In certain embodiments, each coupling independently has a stereoselectivity less than about 60%, 70%, 80%, 85%, or 90%. In certain embodiments, each non-chirally controlled internucleotidic linkage is independently formed with a stereoselectivity less than about 60%, 70%, 80%, 85%, or 90%. In certain embodiments, a stereoselectivity is less than about 60%. In certain embodiments, a stereoselectivity is less than about 70%. In certain embodiments, a stereoselectivity is less than about 80%. In certain embodiments, a stereoselectivity is less than about 90%. In certain embodiments, at least 1, 2, 3, 4, 5, 6, 7, 8, 9, 10, 11, 12, 13, 14, 15, 16, 17, 18, 19, 20, 21, 22, 23, 24, or 25 couplings independently have a stereoselectivity less than about 90%. In certain embodiments, at least one coupling has a stereoselectivity less than about 90%. In certain embodiments, at least two couplings have a stereoselectivity less than about 90%. In certain embodiments, at least three couplings have a stereoselectivity less than about 90%. In certain embodiments, at least four couplings have a stereoselectivity less than about 90%. In certain embodiments, at least five couplings have a stereoselectivity less than about 90%. In certain embodiments, each coupling independently has a stereoselectivity less than about 90%. In certain embodiments, at least 1, 2, 3, 4, 5, 6, 7, 8, 9, 10, 11, 12, 13, 14, 15, 16, 17, 18, 19, 20, 21, 22, 23, 24, or 25 couplings independently have a stereoselectivity less than about 85%. In certain embodiments, each coupling independently has a stereoselectivity less than about 85%. In certain embodiments, at least 1, 2, 3, 4, 5, 6, 7, 8, 9, 10, 11, 12, 13, 14, 15, 16, 17, 18, 19, 20, 21, 22, 23, 24, or 25 couplings independently have a stereoselectivity less than about 80%. In certain embodiments, each coupling independently has a stereoselectivity less than about 80%. In certain embodiments, at least 1, 2, 3, 4, 5, 6, 7, 8, 9, 10, 11, 12, 13, 14, 15, 16, 17, 18, 19, 20, 21, 22, 23, 24, or 25 couplings independently have a stereoselectivity less than about 70%. In certain embodiments, each coupling independently has a stereoselectivity less than about 70%.

In certain embodiments, ds oligonucleotides and compositions of the present disclosure have high purity. In certain embodiments, ds oligonucleotides and compositions of the present disclosure have high stereochemical purity. In certain embodiments, a stereochemical purity, e.g., diastereomeric purity, is about 60%-100%. In certain embodiments, a diastereomeric purity, is about 60%-100%. In certain embodiments, the percentage is at least 60%, 65%, 70%, 75%, 80%, 85%, 90%, 91%, 92%, 93%, 93%, 95%, 96%, 97%, 98%, or 99%. In certain embodiments, the percentage is at least 80%, 85%, 90%, 91%, 92%, 93%, 93%, 95%, 96%, 97%, 98%, or 99%. In certain embodiments, the percentage is at least 90%, 91%, 92%, 93%, 93%, 95%, 96%, 97%, 98%, or 99%. In certain embodiments, a diastereomeric purity is at least 60%. In certain embodiments, a diastereomeric purity is at least 70%. In certain embodiments, a diastereomeric purity is at least 80%. In certain embodiments, a diastereomeric purity is at least 85%. In certain embodiments, a diastereomeric purity is at least 90%. In certain embodiments, a diastereomeric purity is at least 91%. In certain embodiments, a diastereomeric purity is at least 92%. In certain embodiments, a diastereomeric purity is at least 93%. In certain embodiments, a diastereomeric purity is at least 94%. In certain embodiments, a diastereomeric purity is at least 95%. In certain embodiments, a diastereomeric purity is at least 96%. In certain embodiments, a diastereomeric purity is at least 97%. In certain embodiments, a diastereomeric purity is at least 98%. In certain embodiments, a diastereomeric purity is at least 99%. In certain embodiments, a diastereomeric purity is at least 99.5%.

In certain embodiments, compounds of the present disclosure (e.g., oligonucleotides, chiral auxiliaries, etc.) comprise multiple chiral elements (e.g., multiple carbon and/or phosphorus (e.g., linkage phosphorus of chiral internucleotidic linkages) chiral centers). In certain embodiments, at least 1, 2, 3, 4, 5, 6, 7, 8, 9 or more chiral elements of a provided compound (e.g., a ds oligonucleotide) each independently have a diastereomeric purity as described herein. In certain embodiments, at least 1, 2, 3, 4, 5, 6, 7, 8, 9 or more chiral carbon centers of a provided compound each independently have a diastereomeric purity as described herein. In certain embodiments, at least 1, 2, 3, 4, 5, 6, 7, 8, 9 or more chiral phosphorus centers of a provided compound each independently have a diastereomeric purity as described herein. In certain embodiments, each chiral element independently has a diastereomeric purity as described herein. In certain embodiments, each chiral center independently has a diastereomeric purity as described herein. In certain embodiments, each chiral carbon center independently has a diastereomeric purity as described herein. In certain embodiments, each chiral phosphorus center independently has a diastereomeric purity as described herein. In certain embodiments, each chiral phosphorus center independently has a diastereomeric purity of at least 90%, 91%, 92%, 93%, 93%, 95%, 96%, 97%, 98%, or 99% r more.

As understood by a person having ordinary skill in the art, in certain embodiments, diastereoselectivity of a coupling or diastereomeric purity of a chiral linkage phosphorus center can be assessed through the diastereoselectivity of a dimer formation or diastereomeric purity of a dimer prepared under the same or comparable conditions, wherein the dimer has the same 5′- and 3′-nucleosides and internucleotidic linkage.

Various technologies can be utilized for identifying or confirming stereochemistry of chiral elements (e.g., configuration of chiral linkage phosphorus) and/or patterns of backbone chiral centers, and/or for assessing stereoselectivity (e.g., diastereoselectivity of couple steps in oligonucleotide synthesis) and/or stereochemical purity (e.g., diastereomeric purity of internucleotidic linkages, compounds (e.g., oligonucleotides), etc.). Example technologies include NMR [e.g., 1D (one-dimensional) and/or 2D (two-dimensional) ¹H-³¹P HETCOR (heteronuclear correlation spectroscopy)], HPLC, RP-HPLC, mass spectrometry, LC-MS, and cleavage of internucleotidic linkages by stereospecific nucleases, etc., which may be utilized individually or in combination. Example useful nucleases include benzonase, micrococcal nuclease, and svPDE (snake venom phosphodiesterase), which are specific for certain internucleotidic linkages with Rp linkage phosphorus (e.g., a Rp phosphorothioate linkage); and nuclease P1, mung bean nuclease, and nuclease S1, which are specific for internucleotidic linkages with Sp linkage phosphorus (e.g., a Sp phosphorothioate linkage). Without wishing to be bound by any particular theory, the present disclosure notes that, in at least some cases, cleavage of oligonucleotides by a particular nuclease may be impacted by structural elements, e.g., chemical modifications (e.g., 2′-modifications of a sugars), base sequences, or stereochemical contexts. For example, it is observed that in some cases, benzonase and micrococcal nuclease, which are specific for internucleotidic linkages with Rp linkage phosphorus, were unable to cleave an isolated Rp phosphorothioate internucleotidic linkage flanked by Sp phosphorothioate internucleotidic linkages.

In certain embodiments, ds oligonucleotides sharing a common base sequence, a common pattern of backbone linkages, and a common pattern of backbone chiral centers share a common pattern of backbone phosphorus modifications and a common pattern of base modifications. In certain embodiments, sd oligonucleotide compositions sharing a common base sequence, a common pattern of backbone linkages, and a common pattern of backbone chiral centers share a common pattern of backbone phosphorus modifications and a common pattern of nucleoside modifications. In certain embodiments, ds oligonucleotides share a common base sequence, a common pattern of backbone linkages, and a common pattern of backbone chiral centers have identical structures.

In certain embodiments, the present disclosure provides a ds oligonucleotide composition comprising a plurality of oligonucleotides capable of directing RNAi knockdown, wherein ds oligonucleotides of the plurality are of a particular ds oligonucleotide type, which composition is chirally controlled in that it is enriched, relative to a substantially racemic preparation of ds oligonucleotides having the same base sequence, for ds oligonucleotides of the particular ds oligonucleotide type.

In certain embodiments, ds oligonucleotides having a common base sequence, a common pattern of backbone linkages, and a common pattern of backbone chiral centers have a common pattern of backbone phosphorus modifications and a common pattern of base modifications. In certain embodiments, ds oligonucleotides having a common base sequence, a common pattern of backbone linkages, and a common pattern of backbone chiral centers have a common pattern of backbone phosphorus modifications and a common pattern of nucleoside modifications. In certain embodiments, ds oligonucleotides having a common base sequence, a common pattern of backbone linkages, and a common pattern of backbone chiral centers have identical structures.

In certain embodiments, the present disclosure provides dsRNAi oligonucleotide compositions comprising a plurality of oligonucleotides. In certain embodiments, the present disclosure provides chirally controlled oligonucleotide compositions of dsRNAi oligonucleotides. In certain embodiments, the present disclosure provides a dsRNAi oligonucleotide whose base sequence is or is complementary to a dsRNAi sequence disclosed herein or a portion thereof (e.g., various bases sequences in Table 1, wherein each T may be independently replaced with U and vice versa). In certain embodiments, the present disclosure provides a dsRNAi oligonucleotide whose base sequence comprises a base sequence that is or is complementary to a dsRNAi sequence disclosed herein or a portion thereof (e.g., various bases sequences in Table 1). In certain embodiments, the present disclosure provides a dsRNAi oligonucleotide whose base sequence comprises 15 contiguous bases of a base sequence that is or is complementary to a dsRNAi sequence disclosed herein or a portion thereof (e.g., various bases sequences in Table 1, wherein each T may be independently replaced with U and vice versa). In certain embodiments, the present disclosure provides a dsRNAi oligonucleotide which has a base sequence comprising 15 contiguous bases with 0-3 mismatches of a base sequence that is or is complementary to a dsRNAi sequence disclosed herein or a portion thereof (e.g., various bases sequences in Table 1, wherein each T may be independently replaced with U and vice versa). In certain embodiments, the present disclosure provides a dsRNAi oligonucleotide composition wherein the dsRNAi oligonucleotides comprise at least one chiral internucleotidic linkage which is not chirally controlled. In certain embodiments, the present disclosure provides a dsRNAi oligonucleotide comprising a non-chirally controlled chiral internucleotidic linkage, wherein the base sequence of the dsRNAi oligonucleotide comprises a base sequence that is or is complementary to a dsRNAi sequence disclosed herein or a portion thereof (e.g., various bases sequences in Table 1, wherein each T may be independently replaced with U and vice versa). In certain embodiments, the present disclosure provides a dsRNAi oligonucleotide composition comprising a non-chirally controlled chiral internucleotidic linkage, wherein the base sequence of the dsRNAi oligonucleotide is a base sequence that is or is complementary to a dsRNAi sequence disclosed herein or a portion thereof (e.g., various bases sequences in Table 1, wherein each T may be independently replaced with U and vice versa). In certain embodiments, the present disclosure provides a RNAi oligonucleotide comprising a non-chirally controlled chiral internucleotidic linkage, wherein the base sequence of the dsRNAi oligonucleotide comprises 15 contiguous bases of a base sequence that is or is complementary to a dsRNAi sequence disclosed herein or a portion thereof (e.g., various bases sequences in Table 1, wherein each T may be independently replaced with U and vice versa). In certain embodiments, the present disclosure provides a dsRNAi oligonucleotide comprising a non-chirally controlled chiral internucleotidic linkage, wherein the base sequence of the dsRNAi oligonucleotides comprises 15 contiguous bases with 0-3 mismatches of a base sequence that is or is complementary to a RNAi sequence disclosed herein or a portion thereof (e.g., various bases sequences in Table 1, wherein each T may be independently replaced with U and vice versa). In certain embodiments, the present disclosure provides a dsRNAi oligonucleotide comprising a chirally controlled chiral internucleotidic linkage, wherein the base sequence of the dsRNAi oligonucleotide comprises a base sequence that is or is complementary to a dsRNAi sequence disclosed herein or a portion thereof (e.g., various bases sequences in Table 1, wherein each T may be independently replaced with U and vice versa). In certain embodiments, the present disclosure provides a dsRNAi oligonucleotide composition comprising a chirally controlled chiral internucleotidic linkage, wherein the base sequence of the RNAi oligonucleotide is a base sequence that is or is complementary to a dsRNAi sequence disclosed herein or a portion thereof (e.g., various bases sequences in Table 1, wherein each T may be independently replaced with U and vice versa). In certain embodiments, the present disclosure provides a dsRNAi oligonucleotide comprising a chirally controlled chiral internucleotidic linkage, wherein the base sequence of the dsRNAi oligonucleotide comprises 15 contiguous bases of a base sequence that is or is complementary to a dsRNAi sequence disclosed herein or a portion thereof (e.g., various bases sequences in Table 1, wherein each T may be independently replaced with U and vice versa). In certain embodiments, the present disclosure provides a RNAi oligonucleotide comprising a chirally controlled chiral internucleotidic linkage, wherein the base sequence of the RNAi oligonucleotides comprises 15 contiguous bases with 0-3 mismatches of abase sequence that is or is complementary to a dsRNAi sequence disclosed herein or a portion thereof (e.g., various bases sequences in Table 1, wherein each T may be independently replaced with U and vice versa).

In certain embodiments, ds oligonucleotides of the same ds oligonucleotide type have a common pattern of backbone phosphorus modifications and a common pattern of nucleoside modifications. In certain embodiments, ds oligonucleotides of the same ds oligonucleotide type have a common pattern of sugar modifications. In certain embodiments, ds oligonucleotides of the same ds oligonucleotide type have a common pattern of base modifications. In certain embodiments, ds oligonucleotides of the same ds oligonucleotide type have a common pattern of nucleoside modifications. In certain embodiments, ds oligonucleotides of the same ds oligonucleotide type have the same constitution. In certain embodiments, ds oligonucleotides of the same ds oligonucleotide type are identical. In certain embodiments, ds oligonucleotides of the same ds oligonucleotide type are of the same ds oligonucleotide (as those skilled in the art will appreciate, such ds oligonucleotides may each independently exist in one of the various forms of the ds oligonucleotide, and may be the same, or different forms of the ds oligonucleotide). In certain embodiments, ds oligonucleotides of the same ds oligonucleotide type are each independently of the same ds oligonucleotide or a pharmaceutically acceptable salt thereof. In certain embodiments, a plurality of ds oligonucleotides or ds oligonucleotides of a particular ds oligonucleotide type in a provided ds oligonucleotide composition are sdRNAi oligonucleotides. In certain embodiments, the present disclosure provides a chirally controlled dsRNAi oligonucleotide composition comprising a plurality of dsRNAi oligonucleotides, wherein the ds oligonucleotides share:
1) a common base sequence;
2) a common pattern of backbone linkages; and
3) the same linkage phosphorus stereochemistry at one or more chiral internucleotidic linkages (chirally controlled internucleotidic linkages), wherein the composition is enriched, relative to a substantially racemic preparation of oligonucleotides sharing the common base sequence and pattern of backbone linkages, for oligonucleotides of the plurality.

In certain embodiments, as used herein, “one or more” or “at least one” is 1-50, 1-40, 1-30, 1-25, 1-20, 1-15, 1-10, 5-50, 5-40, 5-30, 5-25, 5-20, 5-15, 5-10, 1, 2, 3, 4, 5, 6, 7, 8, 9, 10, 11, 12, 13, 14, 15, 16, 17, 18, 19, 20, or more.

In certain embodiments, a ds oligonucleotide type is further defined by: 4) additional chemical moiety, if any.

In certain embodiments, the percentage is at least about 10%. In certain embodiments, the percentage is at least about 20%. In certain embodiments, the percentage is at least about 30%. In certain embodiments, the percentage is at least about 40%. In certain embodiments, the percentage is at least about 50%. In certain embodiments, the percentage is at least about 60%. In certain embodiments, the percentage is at least about 70%. In certain embodiments, the percentage is at least about 75%. In certain embodiments, the percentage is at least about 80%. In certain embodiments, the percentage is at least about 85%. In certain embodiments, the percentage is at least about 90%. In certain embodiments, the percentage is at least about 91%. In certain embodiments, the percentage is at least about 92%. In certain embodiments, the percentage is at least about 93%. In certain embodiments, the percentage is at least about 94%. In certain embodiments, the percentage is at least about 95%. In certain embodiments, the percentage is at least about 96%. In certain embodiments, the percentage is at least about 97%. In certain embodiments, the percentage is at least about 98%. In certain embodiments, the percentage is at least about 99%. In certain embodiments, the percentage is or is greater than (DS)^{nc}, wherein DS and nc are each independently as described in the present disclosure.

In certain embodiments, a plurality of ds oligonucleotides, e.g., dsRNAi oligonucleotides, share the same constitution. In certain embodiments, a plurality of oligonucleotides, e.g., dsRNAi oligonucleotides, are identical (the same stereoisomer). In certain embodiments, a chirally controlled ds oligonucleotide composition, e.g., a chirally controlled dsRNAi oligonucleotide composition, is a stereopure ds oligonucleotide composition wherein ds oligonucleotides of the plurality are identical (the same stereoisomer), and the composition does not contain any other stereoisomers. Those skilled in the art will appreciate that one or more other stereoisomers may exist as impurities as processes, selectivities, purifications, etc. may not achieve completeness.

In certain embodiments, a provided composition is characterized in that when it is contacted with a target nucleic acid (e.g., a transcript (e.g., pre-mRNA, mature mRNA, other types of RNA, etc. that hybridizes with oligonucleotides of the composition)), levels of the target nucleic acid and/or a product encoded thereby is reduced compared to that observed under a reference condition. In certain embodiments, a reference condition is selected from the group consisting of absence of the composition, presence of a reference composition, and combinations thereof. In certain embodiments, a reference condition is absence of the composition. In certain embodiments, a reference condition is presence of a reference composition. In certain embodiments, a reference composition is a composition whose oligonucleotides do not hybridize with the target nucleic acid. In certain embodiments, a reference composition is a composition whose oligonucleotides do not comprise a sequence that is sufficiently complementary to the target nucleic acid. In certain embodiments, a provided composition is a chirally controlled oligonucleotide composition and a reference composition is a non-chirally controlled oligonucleotide composition which is otherwise identical but is not chirally controlled (e.g., a racemic preparation of oligonucleotides of the same constitution as oligonucleotides of a plurality in the chirally controlled oligonucleotide composition).

In certain embodiments, the present disclosure provides a chirally controlled dsRNAi oligonucleotide composition comprising a plurality of dsRNAi oligonucleotides capable of directing RNAi knockdown, wherein the oligonucleotides share:
1) a common base sequence,
2) a common pattern of backbone linkages, and
3) the same linkage phosphorus stereochemistry at one or more (e.g., 1-50, 1-40, 1-30, 1-25, 1-20, 1-15, 1-10, 5-50, 5-40, 5-30, 5-25, 5-20, 5-15, 5-10, 1, 2, 3, 4, 5, 6, 7, 8, 9, 10, 11, 12, 13, 14, 15, 16, 17, 18, 19, 20, or more) chiral internucleotidic linkages (chirally controlled internucleotidic linkages),
wherein the composition is enriched, relative to a substantially racemic preparation of oligonucleotides sharing the common base sequence and pattern of backbone linkages, for oligonucleotides of the plurality, the ds oligonucleotide composition being characterized in that, when it is contacted with a transcript in a dsRNAi knockdown system, knockdown of the transcript is improved relative to that observed under reference conditions selected from the group consisting of absence of the composition, presence of a reference composition, and combinations thereof.

As noted above and understood in the art, in certain embodiments, the base sequence of a ds oligonucleotide may refer to the identity and/or modification status of nucleoside residues (e.g., of sugar and/or base components, relative to standard naturally occurring nucleotides such as adenine, cytosine, guanosine, thymine, and uracil) in the ds oligonucleotide and/or to the hybridization character (i.e., the ability to hybridize with particular complementary residues) of such residues.

As demonstrated herein, ds oligonucleotide structural elements (e.g., patterns of sugar modifications, backbone linkages, backbone chiral centers, backbone phosphorus modifications, etc.) and combinations thereof can provide surprisingly improved properties and/or bioactivities.

In certain embodiments, ds oligonucleotide compositions are capable of reducing the expression, level and/or activity of a target gene or a gene product thereof. In certain embodiments, ds oligonucleotide compositions are capable of reducing in the expression, level and/or activity of a target gene or a gene product thereof by sterically blocking translation after annealing to a target gene mRNA, by cleaving mRNA (pre-mRNA or mature mRNA), and/or by altering or interfering with mRNA splicing. In certain embodiments, provided dsRNAi oligonucleotide compositions are capable of reducing the expression, level and/or activity of a target gene or a gene product thereof. In certain embodiments, provided dsRNAi oligonucleotide compositions are capable of reducing in the expression, level and/or activity of a target gene or a gene product thereof by sterically blocking translation after annealing to a target gene mRNA, by cleaving target mRNA (pre-mRNA or mature mRNA), and/or by altering or interfering with mRNA splicing.

In certain embodiments, a ds oligonucleotide composition, e.g., a dsdRNAi oligonucleotide composition, is a substantially pure preparation of a single ds oligonucleotide stereoisomer, e.g., a dsRNAi oligonucleotide stereoisomer, in that oligonucleotides in the composition that are not of the oligonucleotide stereoisomer are impurities from the preparation process of said ds oligonucleotide stereoisomer, in some case, after certain purification procedures.

In certain embodiments, the present disclosure provides ds oligonucleotides and oligonucleotide compositions that are chirally controlled, and in certain embodiments, stereopure. For instance, in certain embodiments, a provided composition contains non-random or controlled levels of one or more individual oligonucleotide types as described herein. In certain embodiments, oligonucleotides of the same oligonucleotide type are identical.

### 3. Sugars

Various sugars, including modified sugars, can be utilized in accordance with the present disclosure. In certain embodiments, the present disclosure provides sugar modifications and patterns thereof optionally in combination with other structural elements (e.g., internucleotidic linkage modifications and patterns thereof, pattern of backbone chiral centers thereof, etc.) that when incorporated into oligonucleotides can provide improved properties and/or activities.

The most common naturally occurring nucleosides comprise ribose sugars (e.g., in RNA) or deoxyribose sugars (e.g., in DNA) linked to the nucleobases adenosine (A), cytosine (C), guanine (G), thymine (T) or uracil (U). In certain embodiments, a sugar, e.g., various sugars in many oligonucleotides in Table 1 (unless otherwise notes), is a natural DNA sugar (in DNA nucleic acids or oligonucleotides, having the structure of

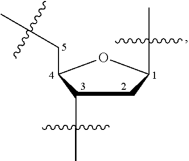 [SMILES: *C[C@H]1O[C@@H](*)C[C@H]1*]

wherein a nucleobase is attached to the 1′ position, and the 3′ and 5′ positions are connected to internucleotidic linkages (as appreciated by those skilled in the art, if at the 5′-end of a ds oligonucleotide, the 5′ position may be connected to a 5′-end group (e.g., —OH), and if at the 3′-end of a ds oligonucleotide, the 3′ position may be connected to a 3′-end group (e.g., —OH). In certain embodiments, a sugar is a natural RNA sugar (in RNA nucleic acids or oligonucleotides, having the structure of

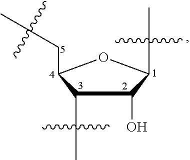

wherein a nucleobase is attached to the 1′ position, and the 3′ and 5′ positions are connected to internucleotidic linkages (as appreciated by those skilled in the art, if at the 5′-end of a ds oligonucleotide, the 5′ position may be connected to a 5′-end group (e.g., —OH), and if at the 3′-end of a ds oligonucleotide, the 3′ position may be connected to a 3′-end group (e.g., —OH). In certain embodiments, a sugar is a modified sugar in that it is not a natural DNA sugar or a natural RNA sugar. Among other things, modified sugars may provide improved stability. In certain embodiments, modified sugars can be utilized to alter and/or optimize one or more hybridization characteristics. In certain embodiments, modified sugars can be utilized to alter and/or optimize target recognition. In certain embodiments, modified sugars can be utilized to optimize Tm. In certain embodiments, modified sugars can be utilized to improve oligonucleotide activities.

Sugars can be bonded to internucleotidic linkages at various positions. As non-limiting examples, internucleotidic linkages can be bonded to the 2′, 3′, 4′ or 5′ positions of sugars. In certain embodiments, as most commonly in natural nucleic acids, an internucleotidic linkage connects with one sugar at the 5′ position and another sugar at the 3′ position unless otherwise indicated.

In certain embodiments, a sugar is an optionally substituted natural DNA or RNA sugar. In certain embodiments, a sugar is optionally substituted

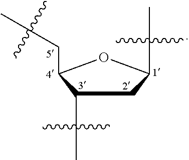 [SMILES: *C[C@H]1O[C@@H](*)C[C@H]1*]

In certain embodiments, the 2′ position is optionally substituted. In certain embodiments, a sugar is

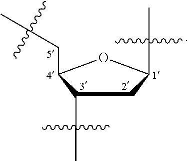 [SMILES: *C[C@H]1O[C@@H](*)C[C@H]1*]

In certain embodiments, a sugar has the structure of

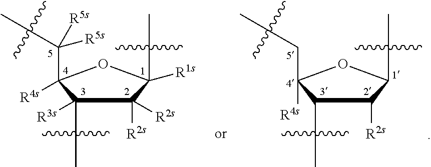

wherein each of R^{1s}, R^{2s}, R^{3s}, R^{4s}, and R^{5s} is independently —H, a suitable substituent or suitable sugar modification (e.g., those described in U.S. Pat. Nos. 9,394,333, 9,744,183, 9,605,019, 9,982,257, US 20170037399, US 20180216108, US 20180216107, U.S. Pat. No. 9,598,458, WO 2017/062862, WO 2018/067973, WO 2017/160741, WO 2017/192679, WO 2017/210647, WO 2018/098264, WO 2018/022473, WO 2018/223056, WO 2018/223073, WO 2018/223081, WO 2018/237194, WO 2019/032607, WO2019/032612, WO 2019/055951, and/or WO 2019/075357, the substituents, sugar modifications, descriptions of R^{1s}, R^{2s}, R^{3s}, R^{4s}, and R^{5s}, and modified sugars of each of which are independently incorporated herein by reference). In certain embodiments, a sugar has the structure of

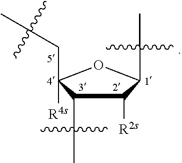

In certain embodiments, R^{4s} is —H. In certain embodiments, a sugar has the structure of

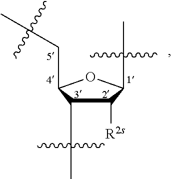

wherein R^{2s} is —H, halogen, or —OR, wherein R is optionally substituted C₁₋₆ aliphatic. In certain embodiments, R^{2s} is —H. In certain embodiments, R^{2s} is —F. In certain embodiments, R^{2s} is —OMe. In certain embodiments, R^{2s} is —OCH₂CH₂OMe.

In certain embodiments, a sugar has the structure of

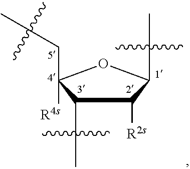

wherein R^{2s} and R^{4s} are taken together to form -L^{s}-, wherein L^{s} is a covalent bond or optionally substituted bivalent C₁₋₆ aliphatic or heteroaliphatic having 1-4 heteroatoms. In certain embodiments, each heteroatom is independently selected from nitrogen, oxygen or sulfur). In certain embodiments, L^{s} is optionally substituted C₂O—CH₂—C₄. In certain embodiments, L^{s} is C₂O—CH₂—C₄. In certain embodiments, L^{s} is C2-O—(R)—CH(CH₂CH₃)—C₄. In certain embodiments, L^{s} is C₂—O—(S)—CH(CH₂CH₃)—C₄.

In certain embodiments, a modified sugar contains one or more substituents at the 2′ position (typically one substituent, and often at the axial position) independently selected from —F; —CF₃, —CN, —N₃, —NO, —NO₂, —OR′, —SR′, or —N(R′)₂, wherein each R′ is independently optionally substituted C₁₋₁₀ aliphatic; —O—(C₁-C₁₀ alkyl), —S—(C₁-C₁₀ alkyl), —NH—(C₁-C₁₀ alkyl), or —N(C₁-C₁₀ alkyl)₂; —O—(C₂-C₁₀ alkenyl), —S—(C₂-C₁₀ alkenyl), —NH—(C₂-C₁₀ alkenyl), or —N(C₂-C₁₀ alkenyl)₂; —O—(C₂-C₁₀ alkynyl), —S—(C₂-C₁₀ alkynyl), —NH—(C₂-C₁₀ alkynyl), or —N(C₂-C₁₀ alkynyl)₂; or —O—(C₁-C₁₀ alkylene)-O—(C₁-C₁₀ alkyl), —O—(C₁-C₁₀ alkylene)-NH—(C₁-C₁₀ alkyl) or —O—(C₁-C₁₀ alkylene)-NH(C₁-C₁₀ alkyl)₂, —NH—(C₁-C₁₀ alkylene)-O—(C₁-C₁₀ alkyl), or —N(C₁-C₁₀ alkyl)-(C₁-C₁₀ alkylene)-O—(C₁-C₁₀ alkyl), wherein each of the alkyl, alkylene, alkenyl and alkynyl is independently and optionally substituted. In certain embodiments, a substituent is —O(CH₂)ₙOCH₃, —O(CH₂)ₙNH₂, MOE, DMAOE, or DMAEOE, wherein n is from 1 to about 10.

In certain embodiments, the 2′-OH of a ribose is replaced with a group selected from —H, —F; —CF₃, —CN, —N₃, —NO, —NO₂, —OR′, —SR′, or —N(R′)₂, wherein each R′ is independently described in the present disclosure; —O—(C₁-C₁₀ alkyl), —S—(C₁-C₁₀ alkyl), —NH—(C₁-C₁₀ alkyl), or —N(C₁-C₁₀ alkyl)₂; —O—(C₂-C₁₀ alkenyl), —S—(C₂-C₁₀ alkenyl), —NH—(C₂-C₁₀ alkenyl), or —N(C₂-C₁₀ alkenyl)₂; —O—(C₂-C₁₀ alkynyl), —S—(C₂-C₁₀ alkynyl), —NH—(C₂-C₁₀ alkynyl), or —N(C₂-C₁₀ alkynyl)₂; or —O—(C₁-C₁₀ alkylene)-O—(C₁-C₁₀ alkyl), —O—(C₁-C₁₀ alkylene)-NH—(C₁-C₁₀ alkyl) or —O—(C₁-C₁₀ alkylene)-NH(C₁-C₁₀ alkyl)₂, —NH—(C₁-C₁₀ alkylene)-O—(C₁-C₁₀ alkyl), or —N(C₁-C₁₀ alkyl)-(C₁—C₁₀ alkylene)-O—(C₁-C₁₀ alkyl), wherein each of the alkyl, alkylene, alkenyl and alkynyl is independently and optionally substituted. In certain embodiments, the 2′-OH is replaced with —H (deoxyribose). In certain embodiments, the 2′-OH is replaced with —F. In certain embodiments, the 2′-OH is replaced with —OR′. In certain embodiments, the 2′-OH is replaced with —OMe. In certain embodiments, the 2′-OH is replaced with —OCH₂CH₂OMe.

In certain embodiments, a sugar modification is a 2′-modification. Commonly used 2′-modifications include but are not limited to 2′-OR, wherein R is optionally substituted C₁₋₆ aliphatic. In certain embodiments, a modification is 2′-OR, wherein R is optionally substituted C₁₋₆ alkyl. In certain embodiments, a modification is 2′-OMe. In certain embodiments, a modification is 2′-MOE. In certain embodiments, a 2′-modification is S-cEt. In certain embodiments, a modified sugar is an LNA sugar. In certain embodiments, a 2′-modification is —F.

In certain embodiments, a sugar modification replaces a sugar moiety with another cyclic or acyclic moiety. Examples of such moieties are widely known in the art, including but not limited to those used in morpholino (optionally with its phosphorodiamidate linkage), glycol nucleic acids, etc.

In certain embodiments, one or more of the sugars of an ATXN3 oligonucleotide are modified. In certain embodiments, each sugar of a ds oligonucleotide is independently modified. In certain embodiments, a modified sugar comprises a 2′-modification. In certain embodiments, each modified sugar independently comprises a 2′-modification. In certain embodiments, a 2′-modification is 2′-OR, wherein R is optionally substituted C₁₋₆ aliphatic. In certain embodiments, a 2′-modification is a 2′-O—C16 lipid modification. In certain embodiments, a 2′-modification is a 2′-OMe. In certain embodiments, a 2′-modification is a 2′-MOE. In certain embodiments, a 2′-modification is an LNA sugar modification. In certain embodiments, a 2′-modification is 2′-F. In certain embodiments, each sugar modification is independently a 2′-modification. In certain embodiments, each sugar modification is independently 2′-OR. In certain embodiments, each sugar modification is independently 2′-OR, wherein R is optionally substituted C₁₋₆ alkyl. In certain embodiments, each sugar modification is a 2′-O—C16 lipid modification. In certain embodiments, each sugar modification is 2′-OMe. In certain embodiments, each sugar modification is 2′-MOE. In certain embodiments, each sugar modification is independently 2′-OMe, a 2′-O—C16 lipid modification or 2′-MOE. In certain embodiments, each sugar modification is independently 2′-OMe, 2′-MOE, or a LNA sugar.

In certain embodiments, a modified sugar comprises a 5′-modification. In certain embodiments, each modified sugar independently comprises a 5′-modification. In certain embodiments, a 5′-modification is 5′-alkyl modification. In certain embodiments, a 5′-alkyl modification is a 5′-methyl modification. In particular embodiments, a 5′-methyl modification is a 5′-(R)-methyl modification. In certain embodiments, a 5′-methyl modification is a 5′-(S)-methyl modification.

As those skilled in the art will appreciate, modifications of sugars, nucleobases, internucleotidic linkages, etc. can and are often utilized in combination in oligonucleotides, e.g., see various oligonucleotides in Table 1. For example, a combination of sugar modification and nucleobase modification is 2′-F (sugar) 5-methyl (nucleobase) modified nucleosides. In certain embodiments, a combination is replacement of a ribosyl ring oxygen atom with S and substitution at the 2′-position.

In certain embodiments, a sugar is one described in U.S. Pat. Nos. 9,394,333, 9,744,183, 9,605,019, 9,598,458, 9,982,257, U.S. Ser. No. 10/160,969, U.S. Ser. No. 10/479,995, US 2020/0056173, US 2018/0216107, US 2019/0127733, U.S. Ser. No. 10/450,568, US 2019/0077817, US 2019/0249173, US 2019/0375774, WO 2018/223056, WO 2018/223073, WO 2018/223081, WO 2018/237194, WO 2019/032607, WO 2019/055951, WO 2019/075357, WO 2019/200185, WO 2019/217784, and/or WO 2019/032612, the sugars of each of which is incorporated herein by reference.

Various additional sugars useful for preparing oligonucleotides or analogs thereof are known in the art and may be utilized in accordance with the present disclosure.

### 4. Nucleobases

Various nucleobases may be utilized in provided ds oligonucleotides in accordance with the present disclosure. In certain embodiments, a nucleobase is a natural nucleobase, the most commonly occurring ones being A, T, C, G and U. In certain embodiments, a nucleobase is a modified nucleobase in that it is not A, T, C, G or U. In certain embodiments, a nucleobase is optionally substituted A, T, C, G or U, or a substituted tautomer of A T, C, G or U. In certain embodiments, a nucleobase is optionally substituted A, T, C, G or U, e.g., 5mC, 5-hydroxymethyl C, etc. In certain embodiments, a nucleobase is alkyl-substituted A, T, C, G or U. In certain embodiments, a nucleobase is A. In certain embodiments, a nucleobase is T. In certain embodiments, a nucleobase is C. In certain embodiments, a nucleobase is G. In certain embodiments, a nucleobase is U. In certain embodiments, a nucleobase is 5mC. In certain embodiments, a nucleobase is substituted A, T, C, G or U. In certain embodiments, a nucleobase is a substituted tautomer of A, T, C, G or U. In certain embodiments, substitution protects certain functional groups in nucleobases to minimize undesired reactions during oligonucleotide synthesis. Suitable technologies for nucleobase protection in oligonucleotide synthesis are widely known in the art and may be utilized in accordance with the present disclosure. In certain embodiments, modified nucleobases improves properties and/or activities of ds oligonucleotides. For example, in many cases, 5mC may be utilized in place of C to modulate certain undesired biological effects, e.g., immune responses. In certain embodiments, when determining sequence identity, a substituted nucleobase having the same hydrogen-bonding pattern is treated as the same as the unsubstituted nucleobase, e.g., 5mC may be treated the same as C [e.g., a ds oligonucleotide having 5mC in place of C (e.g., AT5mCG) is considered to have the same base sequence as a ds oligonucleotide having C at the corresponding location(s) (e.g., ATCG)].

In certain embodiments, a ds oligonucleotide comprises one or more A, T, C, G or U. In certain embodiments, a ds oligonucleotide comprises one or more optionally substituted A, T, C, G or U. In certain embodiments, a ds oligonucleotide comprises one or more 5-methylcytidine, 5-hydroxymethylcytidine, 5-formylcytosine, or 5-carboxylcytosine. In certain embodiments, a ds oligonucleotide comprises one or more 5-methylcytidine. In certain embodiments, each nucleobase in a ds oligonucleotide is selected from the group consisting of optionally substituted A, T, C, G and U, and optionally substituted tautomers of A, T, C, G and U.

In certain embodiments, each nucleobase in a ds oligonucleotide is optionally protected A, T, C, G and U. In certain embodiments, each nucleobase in a ds oligonucleotide is optionally substituted A, T, C, G or U. In certain embodiments, each nucleobase in a ds oligonucleotide is selected from the group consisting of A, T, C, G, U, and 5mC.

In certain embodiments, a nucleobase is optionally substituted 2AP or DAP. In certain embodiments, a nucleobase is optionally substituted 2AP. In certain embodiments, a nucleobase is optionally substituted DAP. In certain embodiments, a nucleobase is 2AP. In certain embodiments, a nucleobase is DAP.

In certain embodiments, a nucleobase is a natural nucleobase or a modified nucleobase derived from a natural nucleobase. Examples include uracil, thymine, adenine, cytosine, and guanine optionally having their respective amino groups protected by acyl protecting groups, 2-fluorouracil, 2-fluorocytosine, 5-bromouracil, 5-iodouracil, 2,6-diaminopurine, azacytosine, pyrimidine analogs such as pseudoisocytosine and pseudouracil and other modified nucleobases such as 8-substituted purines, xanthine, or hypoxanthine (the latter two being the natural degradation products). Certain examples of modified nucleobases are disclosed in Chiu and Rana, R N A, 2003, 9, 1034-1048, Limbach et al. Nucleic Acids Research, 1994, 22, 2183-2196 and Revankar and Rao, Comprehensive Natural Products Chemistry, vol. 7, 313. In certain embodiments, a modified nucleobase is substituted uracil, thymine, adenine, cytosine, or guanine. In certain embodiments, a modified nucleobase is a functional replacement, e.g., in terms of hydrogen bonding and/or base pairing, of uracil, thymine, adenine, cytosine, or guanine. In certain embodiments, a nucleobase is optionally substituted uracil, thymine, adenine, cytosine, 5-methylcytosine, or guanine. In certain embodiments, a nucleobase is uracil, thymine, adenine, cytosine, 5-methylcytosine, or guanine.

In certain embodiments, a provided ds oligonucleotide comprises one or more 5-methylcytosine. In certain embodiments, the present disclosure provides a ds oligonucleotide whose base sequence is disclosed herein, e.g., in Table 1, wherein each T may be independently replaced with U and vice versa, and each cytosine is optionally and independently replaced with 5-methylcytosine or vice versa. As appreciated by those skilled in the art, in certain embodiments, 5mC may be treated as C with respect to base sequence of an oligonucleotide—such oligonucleotide comprises a nucleobase modification at the C position (e.g., see various oligonucleotides in Table 1). In description of oligonucleotides, typically unless otherwise noted, nucleobases, sugars and internucleotidic linkages are non-modified.

In certain embodiments, a modified base is optionally substituted adenine, cytosine, guanine, thymine, or uracil, or a tautomer thereof. In certain embodiments, a modified nucleobase is a modified adenine, cytosine, guanine, thymine or uracil, modified by one or more modifications by which:
1) a nucleobase is modified by one or more optionally substituted groups independently selected from acyl, halogen, amino, azide, alkyl, alkenyl, alkynyl, aryl, heteroalkyl, heteroalkenyl, heteroalkynyl, heterocyclyl, heteroaryl, carboxyl, hydroxyl, biotin, avidin, streptavidin, substituted silyl, and combinations thereof;
2) one or more atoms of a nucleobase are independently replaced with a different atom selected from carbon, nitrogen and sulfur;
3) one or more double bonds in a nucleobase are independently hydrogenated; or
4) one or more aryl or heteroaryl rings are independently inserted into a nucleobase.

In certain embodiments, a modified nucleobase is a modified nucleobase known in the art, e.g., WO2017/210647. In certain embodiments, modified nucleobases are expanded-size nucleobases in which one or more aryl and/or heteroaryl rings, such as phenyl rings, have been added.

In certain embodiments, a modified nucleobase is selected from 5-substituted pyrimidines, 6-azapyrimidines, alkyl or alkynyl substituted pyrimidines, alkyl substituted purines, and N-2, N-6 and 0-6 substituted purines. In certain embodiments, modified nucleobases are selected from 2-aminopropyladenine, 5-hydroxymethyl cytosine, xanthine, hypoxanthine, 2-aminoadenine, 6-N-methylguanine, 6-N-methyladenine, 2-propyladenine, 2-thiouracil, 2-thiothymine and 2-thiocytosine, 5-propynyl (—C≡C—CH₃) uracil, 5-propynylcytosine, 6-azouracil, 6-azocytosine, 6-azothymine, 5-ribosyluracil (pseudouracil), 4-thiouracil, 8-halo, 8-amino, 8-thiol, 8-thioalkyl, 8-hydroxyl, 8-aza and other 8-substituted purines, 5-halo, particularly 5-bromo, 5-trifluoromethyl, 5-halouracil, and 5-halocytosine, 7-methylguanine, 7-methyladenine, 2-F-adenine, 2-aminoadenine, 7-deazaguanine, 7-deazaadenine, 3-deazaguanine, 3-deazaadenine, 6-N— benzoyladenine, 2-N-isobutyrylguanine, 4-N-benzoylcytosine, 4-N-benzoyluracil, 5-methyl 4-N— benzoylcytosine, 5-methyl 4-N— benzoyluracil, universal bases, hydrophobic bases, promiscuous bases, size-expanded bases, and fluorinated bases. In certain embodiments, modified nucleobases are tricyclic pyrimidines, such as 1,3-diazaphenoxazine-2-one, 1,3-diazaphenothiazine-2-one or 9-(2-aminoethoxy)-1,3-diazaphenoxazine-2-one (G-clamp). In certain embodiments, modified nucleobases are those in which the purine or pyrimidine base is replaced with other heterocycles, for example, 7-deaza-adenine, 7-deazaguanosine, 2-aminopyridine or 2-pyridone.

In certain embodiments, a modified nucleobase is substituted. In certain embodiments, a modified nucleobase is substituted such that it contains, e.g., heteroatoms, alkyl groups, or linking moieties connected to fluorescent moieties, biotin or avidin moieties, or other protein or peptides. In certain embodiments, a modified nucleobase is a “universal base” that is not a nucleobase in the most classical sense, but that functions similarly to a nucleobase. One example of a universal base is 3-nitropyrrole.

In certain embodiments, nucleosides that can be utilized in provided technologies comprise modified nucleobases and/or modified sugars, e.g., 4-acetylcytidine; 5-(carboxyhydroxylmethyl)uridine; 2′-O— methylcytidine; 5-carboxymethylaminomethyl-2-thiouridine; 5-carboxymethylaminomethyluridine; dihydrouridine; 2′-O— methylpseudouridine; beta,D-galactosylqueosine; 2′-O-methylguanosine; N⁶-isopentenyladenosine; 1-methyladenosine; 1-methylpseudouridine; 1-methylguanosine; 1-methylinosine; 2,2-dimethylguanosine; 2-methyladenosine; 2-methylguanosine; N⁷-methylguanosine; 3-methyl-cytidine; 5-methylcytidine; 5-hydroxymethylcytidine; 5-formylcytosine; 5-carboxylcytosine; N⁶-methyladenosine; 7-methylguanosine; 5-methylaminoethyluridine; 5-methoxyaminomethyl-2-thiouridine; beta,D-mannosylqueosine; 5-methoxycarbonylmethyluridine; 5-methoxyuridine; 2-methylthio-N⁶-isopentenyladenosine; N-((9-beta,D-ribofuranosyl-2-methylthiopurine-6-yl)carbamoyl)threonine; N-((9-beta,D-ribofuranosylpurine-6-yl)-N-methylcarbamoyl)threonine; uridine-5-oxyacetic acid methylester; uridine-5-oxy acetic acid (v); pseudouridine; queosine; 2-thiocytidine; 5-methyl-2-thiouridine; 2-thiouridine; 4-thiouridine; 5-methyluridine; 2′-O-methyl-5-methyluridine; and 2′-O-methyluridine. In certain embodiments, a nucleobase, e.g., a modified nucleobase comprises one or more biomolecule binding moieties such as e.g., antibodies, antibody fragments, biotin, avidin, streptavidin, receptor ligands, or chelating moieties. In other embodiments, a nucleobase is 5-bromouracil, 5 iodouracil, or 2,6-diaminopurine. In certain embodiments, a nucleobase comprises substitution with a fluorescent or biomolecule binding moiety. In certain embodiments, a substituent is a fluorescent moiety.

In certain embodiments, a substituent is biotin or avidin.

In certain embodiments, a nucleobase is one described in U.S. Pat. Nos. 9,394,333, 9,744,183, 9,605,019, 9,598,458, 9,982,257, U.S. Ser. No. 10/160,969, U.S. Ser. No. 10/479,995, US 2020/0056173, US 2018/0216107, US 2019/0127733, U.S. Ser. No. 10/450,568, US 2019/0077817, US 2019/0249173, US 2019/0375774, WO 2018/223056, WO 2018/223073, WO 2018/223081, WO 2018/237194, WO 2019/032607, WO 2019/055951, WO 2019/075357, WO 2019/200185, WO 2019/217784, and/or WO 2019/032612, the nucleobases of each of which is incorporated herein by reference.

### 5. Additional Chemical Moieties

In certain embodiments, a ds oligonucleotide comprises one or more additional chemical moieties. Various additional chemical moieties, e.g., targeting moieties, carbohydrate moieties, lipid moieties, etc. are known in the art and can be utilized in accordance with the present disclosure to modulate properties and/or activities of provided oligonucleotides, e.g., stability, half-life, activities, delivery, pharmacodynamics properties, pharmacokinetic properties, etc. In certain embodiments, certain additional chemical moieties facilitate delivery of oligonucleotides to desired cells, tissues and/or organs, including but not limited the cells of the central nervous system. In certain embodiments, certain additional chemical moieties facilitate internalization of oligonucleotides. In certain embodiments, certain additional chemical moieties increase oligonucleotide stability. In certain embodiments, the present disclosure provides technologies for incorporating various additional chemical moieties into oligonucleotides.

In certain embodiments, a ds oligonucleotide comprises an additional chemical moiety demonstrates increased delivery to and/or activity in a tissue compared to a reference oligonucleotide, e.g., a reference oligonucleotide which does not have the additional chemical moiety but is otherwise identical.

In certain embodiments, non-limiting examples of additional chemical moieties include carbohydrate moieties, targeting moieties, etc., which, when incorporated into oligonucleotides, can improve one or more properties. In certain embodiments, an additional chemical moiety is selected from: glucose, GluNAc (N-acetyl amine glucosamine) and anisamide moieties. In certain embodiments, a provided ds oligonucleotide can comprise two or more additional chemical moieties, wherein the additional chemical moieties are identical or non-identical, or are of the same category (e.g., carbohydrate moiety, sugar moiety, targeting moiety, etc.) or not of the same category.

In certain embodiments, an additional chemical moiety is a targeting moiety. In certain embodiments, an additional chemical moiety is or comprises a carbohydrate moiety. In certain embodiments, an additional chemical moiety is or comprises a lipid moiety. In certain embodiments, an additional chemical moiety is or comprises a ligand moiety for, e.g., cell receptors such as a sigma receptor, an asialoglycoprotein receptor, etc. In certain embodiments, a ligand moiety is or comprises an anisamide moiety, which may be a ligand moiety for a sigma receptor. In certain embodiments, a ligand moiety is or comprises a GalNAc moiety, which may be a ligand moiety for an asialoglycoprotein receptor. In certain embodiments, an additional chemical moiety facilitates delivery to liver.

In certain embodiments, a provided ds oligonucleotide can comprise one or more linkers and additional chemical moieties (e.g., targeting moieties), and/or can be chirally controlled or not chirally controlled, and/or have a bases sequence and/or one or more modifications and/or formats as described herein.

Various linkers, carbohydrate moieties and targeting moieties, including many known in the art, can be utilized in accordance with the present disclosure. In certain embodiments, a carbohydrate moiety is a targeting moiety. In certain embodiments, a targeting moiety is a carbohydrate moiety.

In certain embodiments, a provided ds oligonucleotide comprises an additional chemical moiety suitable for delivery, e.g., glucose, GluNAc (N-acetyl amine glucosamine), anisamide, or a structure selected from:

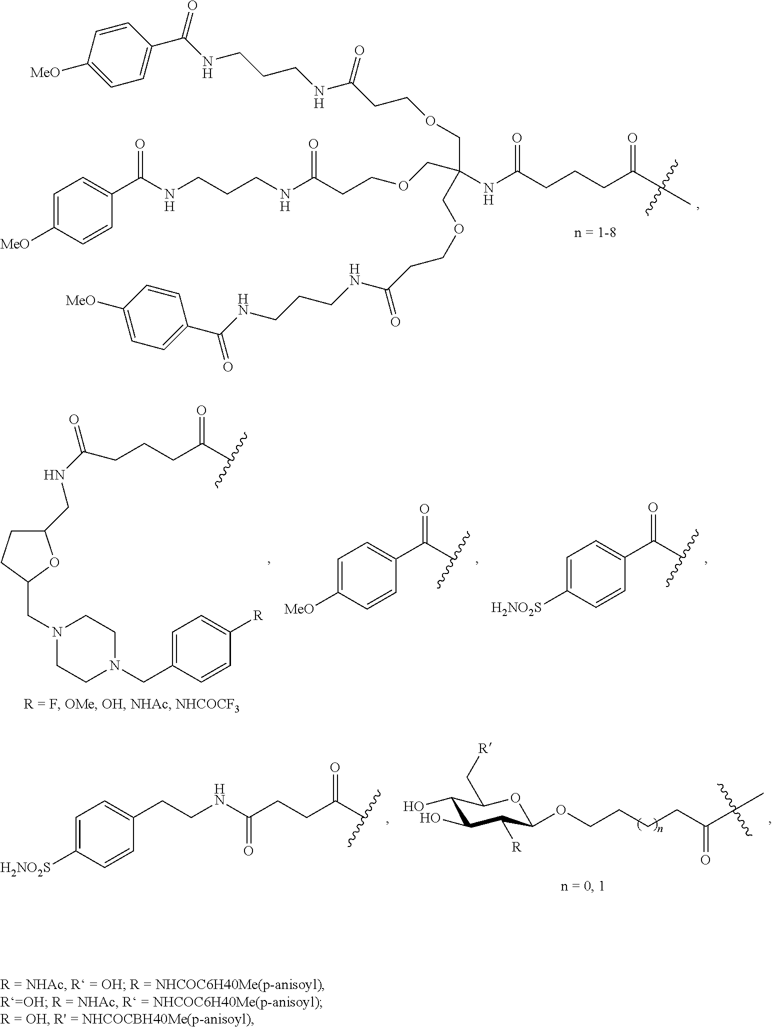

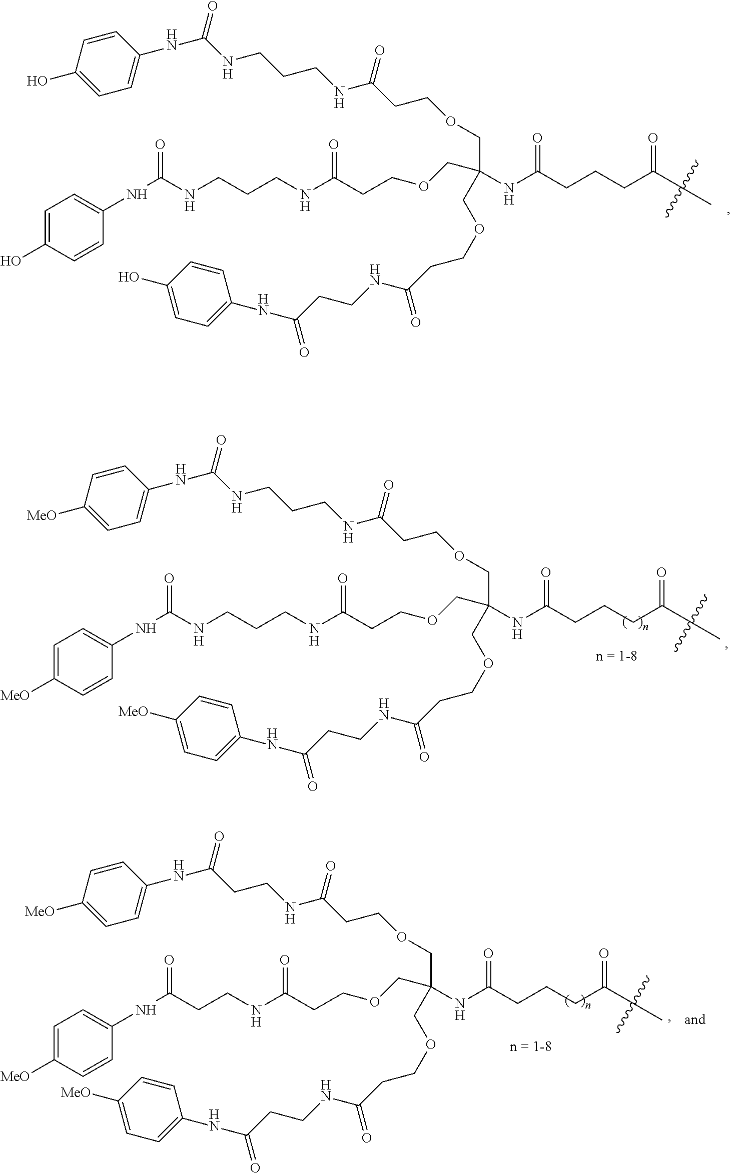

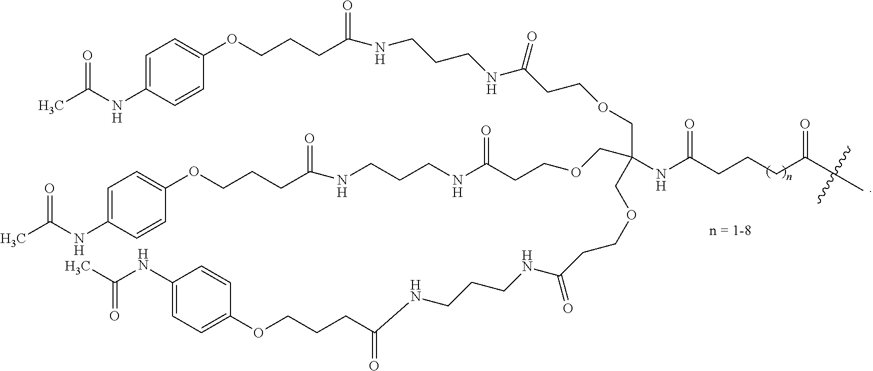

In certain embodiments, n is 1. In certain embodiments, n is 2. In certain embodiments, n is 3. In certain embodiments, n is 4. In certain embodiments, n is 5. In certain embodiments, n is 6. In certain embodiments, n is 7. In certain embodiments, n is 8.

In certain embodiments, additional chemical moieties are any of ones described in the Examples, including examples of various additional chemical moieties incorporated into various ds oligonucleotides.

In certain embodiments, an additional chemical moiety conjugated to a ds oligonucleotide is capable of targeting the ds oligonucleotide to a cell in the central nervous system.

In certain embodiments, an additional chemical moiety comprises or is a cell receptor ligand. In certain embodiments, an additional chemical moiety comprises or is a protein binder, e.g., one binds to a cell surface protein. Such moieties among other things can be useful for targeted delivery of ds oligonucleotides to cells expressing the corresponding receptors or proteins. In certain embodiments, an additional chemical moiety of a provided ds oligonucleotide comprises anisamide or a derivative or an analog thereof and is capable of targeting the ds oligonucleotide to a cell expressing a particular receptor, such as the sigma 1 receptor.

In certain embodiments, a provided ds oligonucleotide is formulated for administration to a body cell and/or tissue expressing its target. In certain embodiments, an additional chemical moiety conjugated to a ds oligonucleotide is capable of targeting the oligonucleotide to a cell.

In certain embodiments, an additional chemical moiety is selected from optionally substituted phenyl,

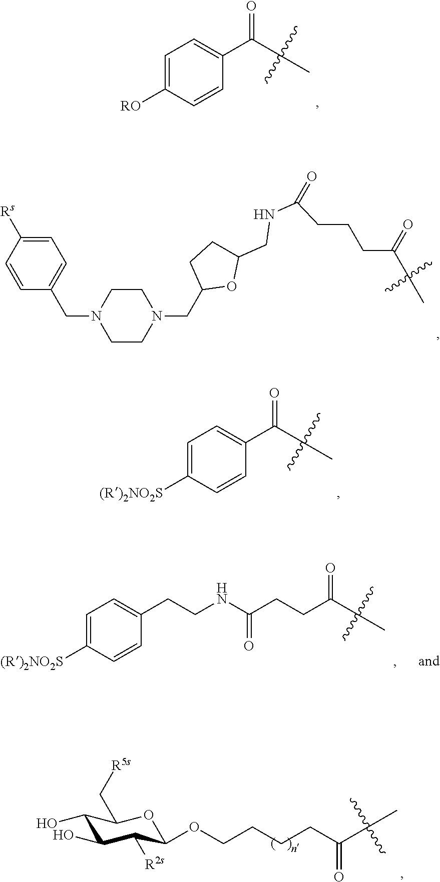

wherein n′ is 1, 2, 3, 4, 5, 6, 7, 8, 9, or 10, and each other variable is as described in the present disclosure. In certain embodiments, R^{s} is F. In certain embodiments, R^{s} is OMe. In certain embodiments, R^{s} is OH. In certain embodiments, R^{s} is NHAc. In certain embodiments, R^{s} is NHCOCF₃. In certain embodiments, R′ is H. In certain embodiments, R is H. In certain embodiments, R^{2s} is NHAc, and R^{5S} is OH. In certain embodiments, R^{2s} is p-anisoyl, and R^{5s} is OH. In certain embodiments, R^{2s} is NHAc and R^{5s} is p-anisoyl. In certain embodiments, R^{2s} is OH, and R^{5s} is p-anisoyl. In certain embodiments, an additional chemical moiety is selected from

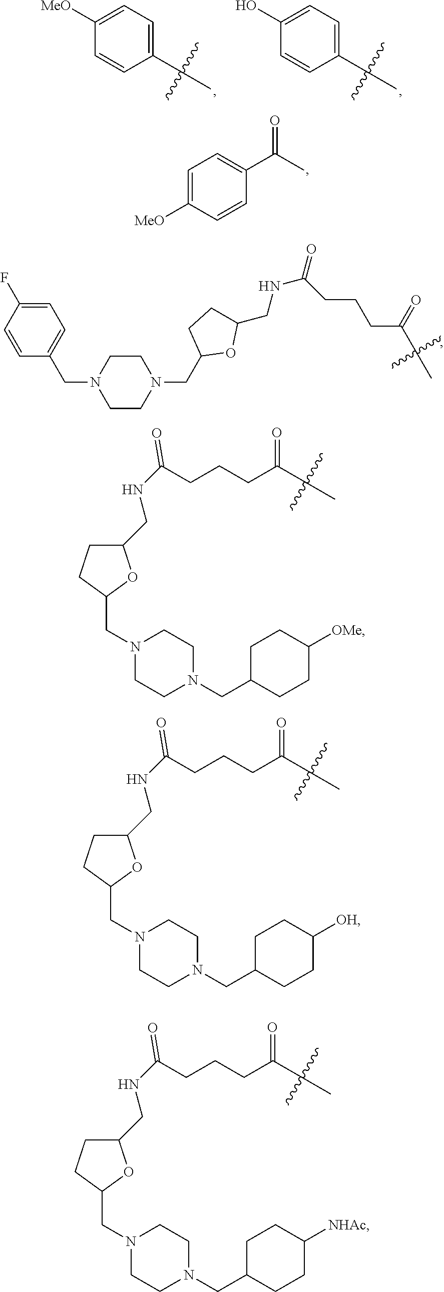

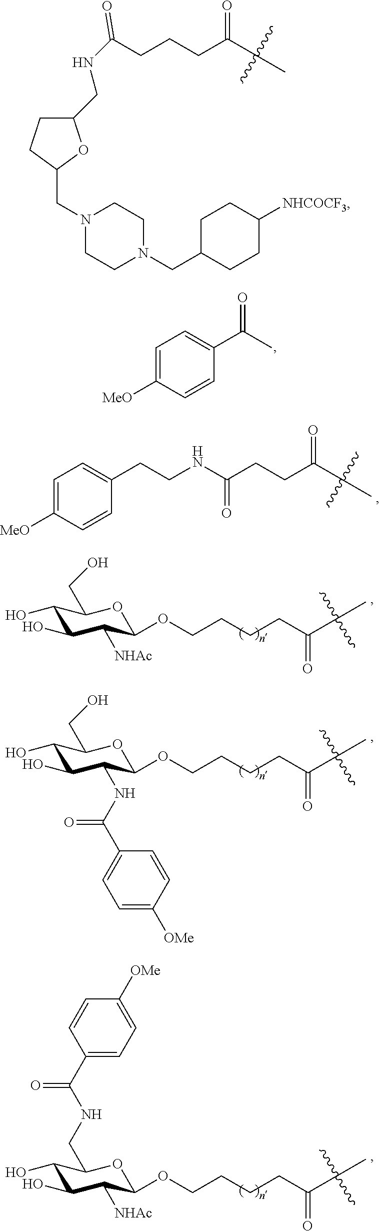

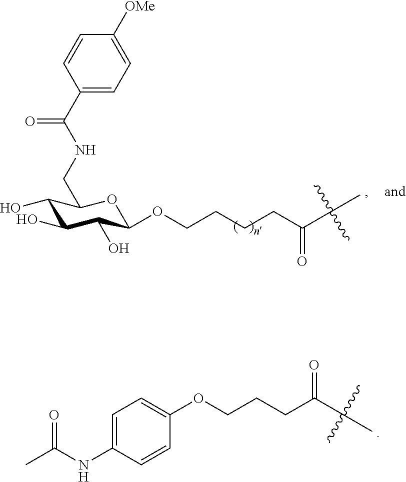

In certain embodiments, n′ is 1. In certain embodiments, n′ is 0. In certain embodiments, n″ is 1. In certain embodiments, n″ is 2.

In certain embodiments, an additional chemical moiety is or comprises an asialoglycoprotein receptor (ASGPR) ligand.

Without wishing to be bound by any particular theory, the present disclosure notes that ASGPR1 has also been reported to be expressed in the hippocampus region and/or cerebellum Purkinje cell layer of the mouse. http://mouse.brain-map.org/experiment/show/2048

Various other ASGPR ligands are known in the art and can be utilized in accordance with the present disclosure. In certain embodiments, an ASGPR ligand is a carbohydrate. In certain embodiments, an ASGPR ligand is GalNac or a derivative or an analog thereof. In certain embodiments, an ASGPR ligand is one described in Sanhueza et al. J. Am. Chem. Soc., 2017, 139 (9), pp 3528-3536. In certain embodiments, an ASGPR ligand is one described in Mamidyala et al. J. Am. Chem. Soc., 2012, 134, pp 1978-1981. In certain embodiments, an ASGPR ligand is one described in US 20160207953. In certain embodiments, an ASGPR ligand is a substituted-6,8-dioxabicyclo[3.2.1]octane-2,3-diol derivative disclosed in, e.g., US 20160207953. In certain embodiments, an ASGPR ligand is one described in, e.g., US 20150329555. In certain embodiments, an ASGPR ligand is a substituted-6,8-dioxabicyclo[3.2.1]octane-2,3-diol derivative disclosed e.g., in US 20150329555. In certain embodiments, an ASGPR ligand is one described in U.S. Pat. No. 8,877,917, US 20160376585, U.S. Ser. No. 10/086,081, or U.S. Pat. No. 8,106,022. ASGPR ligands described in these documents are incorporated herein by reference. Those skilled in the art will appreciate that various technologies are known in the art, including those described in these documents, for assessing binding of a chemical moiety to ASGPR and can be utilized in accordance with the present disclosure. In certain embodiments, a provided ds oligonucleotide is conjugated to an ASGPR ligand. In certain embodiments, a provided ds oligonucleotide comprises an ASGPR ligand. In certain embodiments, an additional chemical moiety comprises an ASGPR ligand is

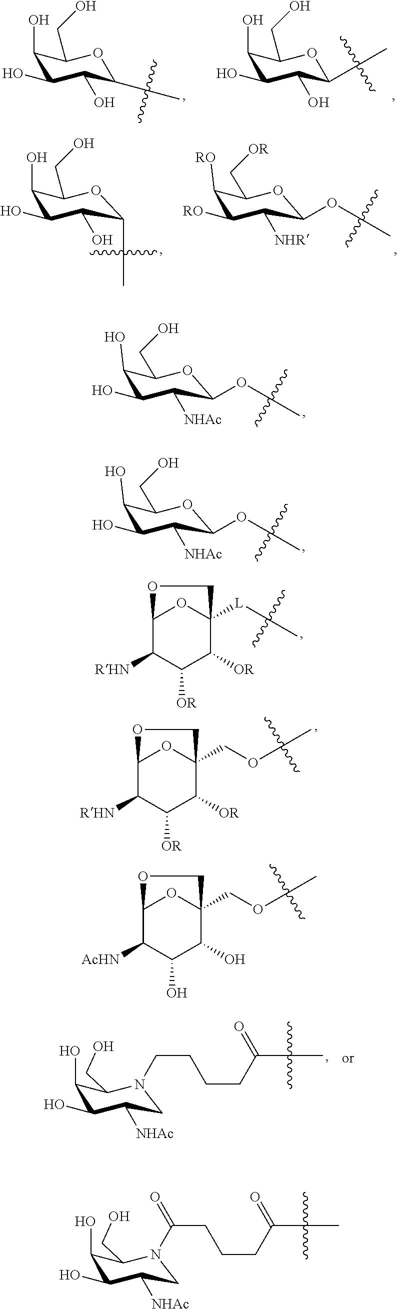

wherein each variable is independently as described in the present disclosure. In certain embodiments, R is —H. In certain embodiments, R′ is —C(O)R.

In certain embodiments, an additional chemical moiety is or comprises

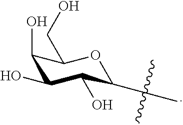

In certain embodiments, an additional chemical moiety is or comprises

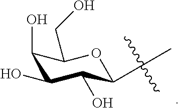

In certain embodiments, an additional chemical moiety is or comprises

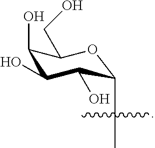

In certain embodiments, an additional chemical moiety is or comprises

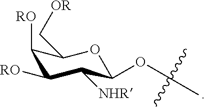

In certain embodiments, an additional chemical moiety is or comprises optionally substituted

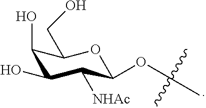

In certain embodiments, an additional chemical moiety is or comprises

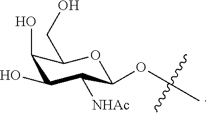

In certain embodiments, an additional chemical moiety is or comprises

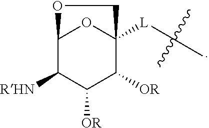

In certain embodiments, an additional chemical moiety is or comprises

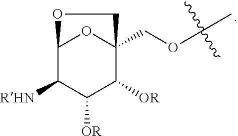

In certain embodiments, an additional chemical moiety is or comprises

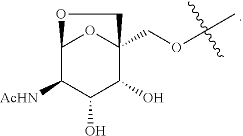 [SMILES: *OC[C@@]12CO[C@@H](O1)[C@H](NC(C)=O)[C@@H](O)[C@H]2O]

In certain embodiments, an additional chemical moiety comprises one or more moieties that can bind to, e.g., oligonucleotide target cells. For example, in certain embodiments, an additional chemistry moiety comprises one or more protein ligand moieties, e.g., in certain embodiments, an additional chemical moiety comprises multiple moieties, each of which independently is an ASGPR ligand.

In particular embodiments, an additional chemical moiety is or comprises a mono-ASGPR ligand with one such moiety. In certain embodiments a mono-ASGPR ligand comprises:

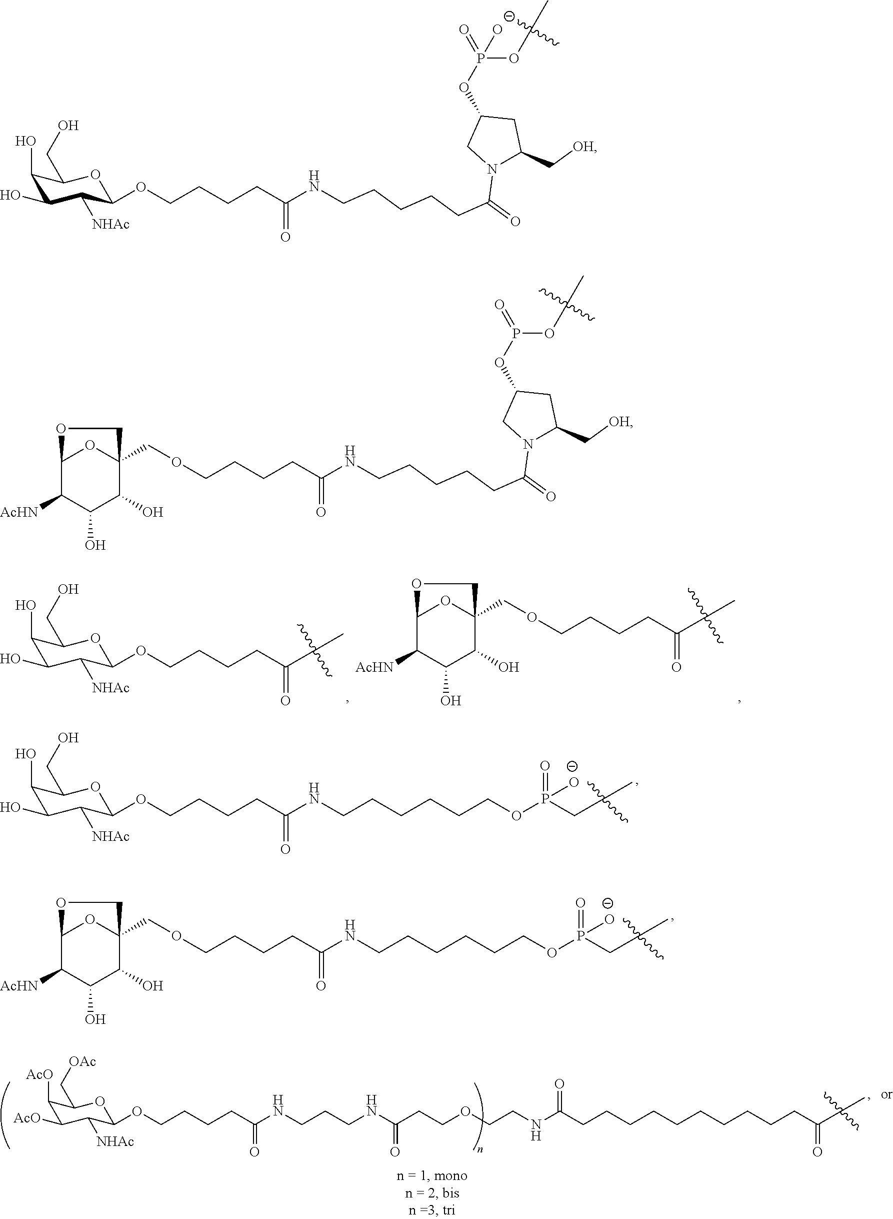

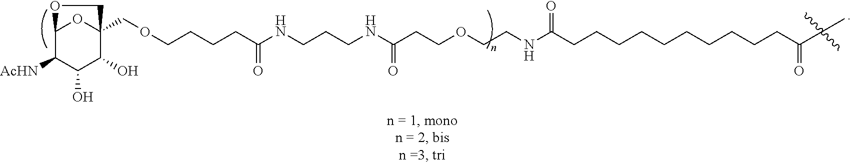

In particular embodiments, an additional chemical moiety is or comprises a bi-ASGPR ligand with two such moieties. In certain embodiments a bi-ASGPR ligand comprises:

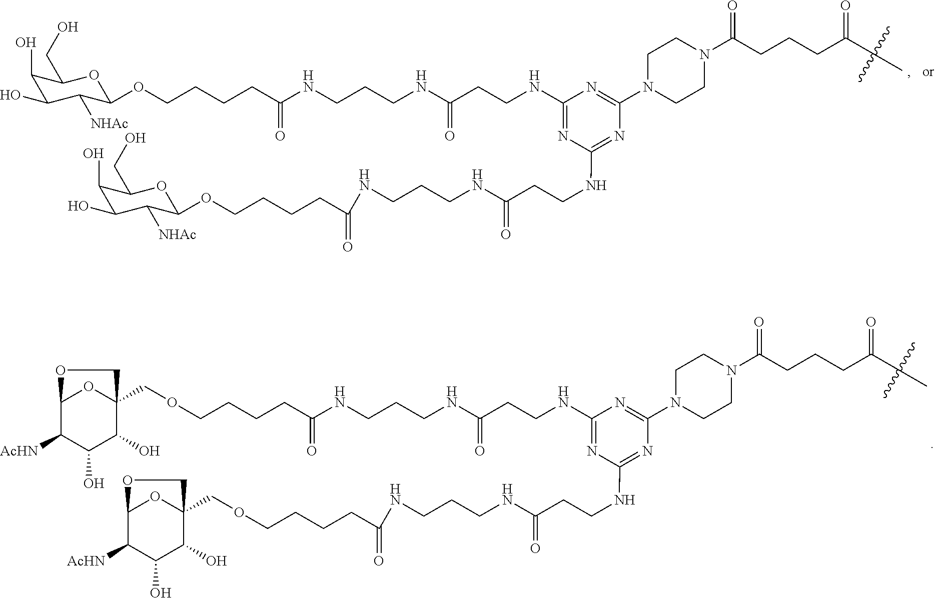 [SMILES: *C(=O)CCCC(=O)N1CCN(c2nc(NCCC(=O)NCCCNC(=O)CCCCOC3OC(CO)C(O)C(O)C3NC(C)=O)nc(NCCC(=O)NCCCNC(=O)CCCCOC3OC(CO)C(O)C(O)C3NC(C)=O)n2)CC1 ; *C(=O)CCCC(=O)N1CCN(c2nc(NCCC(=O)NCCCNC(=O)CCCCOC[C@@]34CO[C@@H](O3)[C@H](NC(C)=O)[C@@H](O)[C@H]4O)nc(NCCC(=O)NCCCNC(=O)CCCCOC[C@@]34CO[C@@H](O3)[C@H](NC(C)=O)[C@@H](O)[C@H]4O)n2)CC1]

In particular embodiments, an additional chemical moiety is or comprises a tri-antennary ASGPR ligand with three such moieties. In certain embodiments a tri-antennary ASGPR ligand comprises:

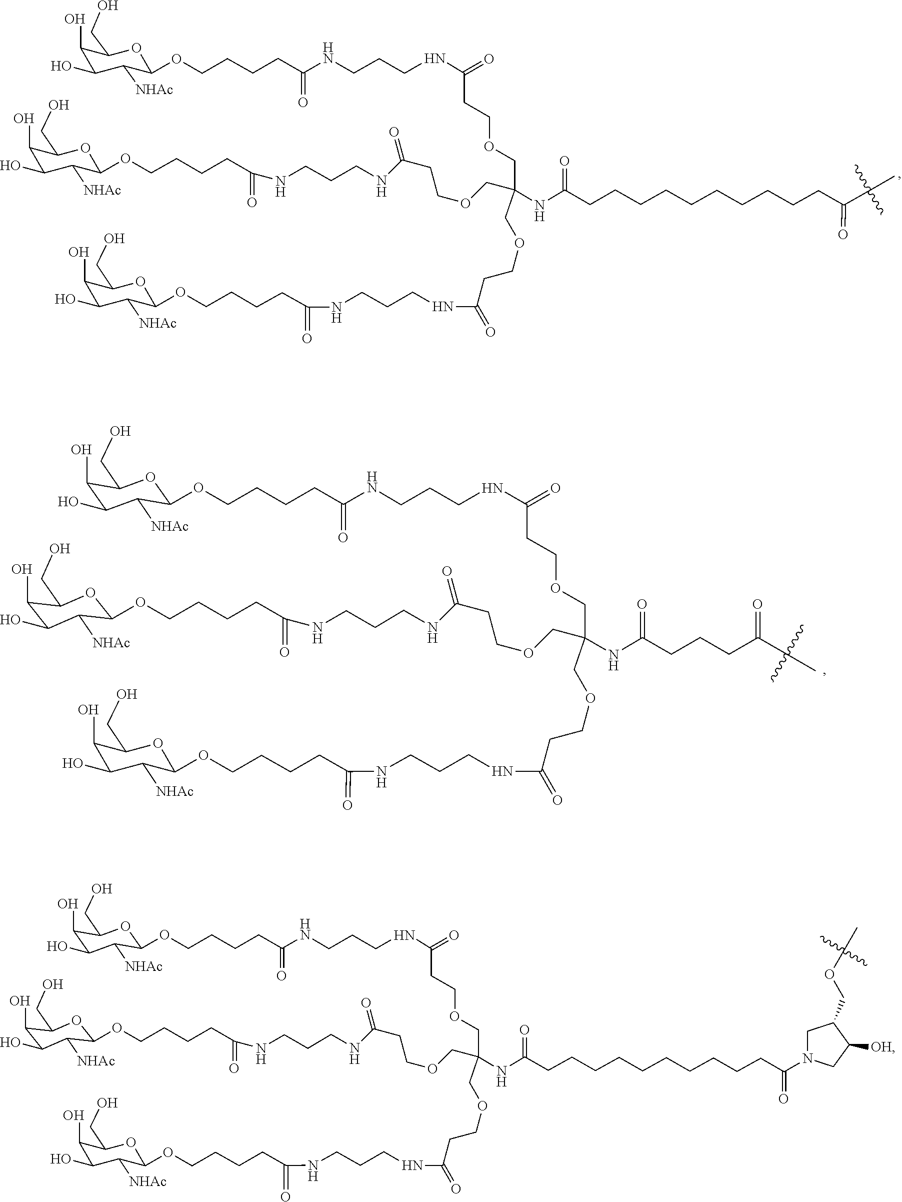

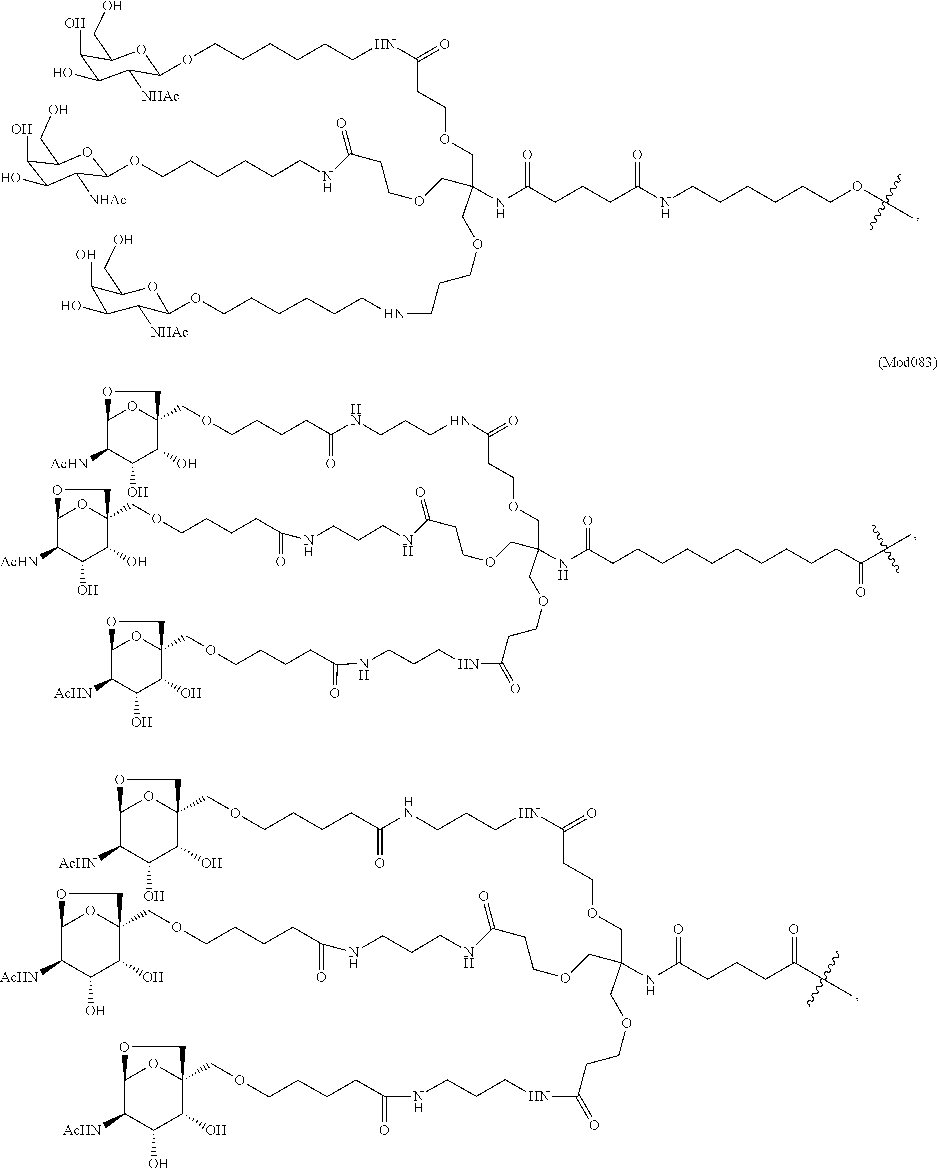

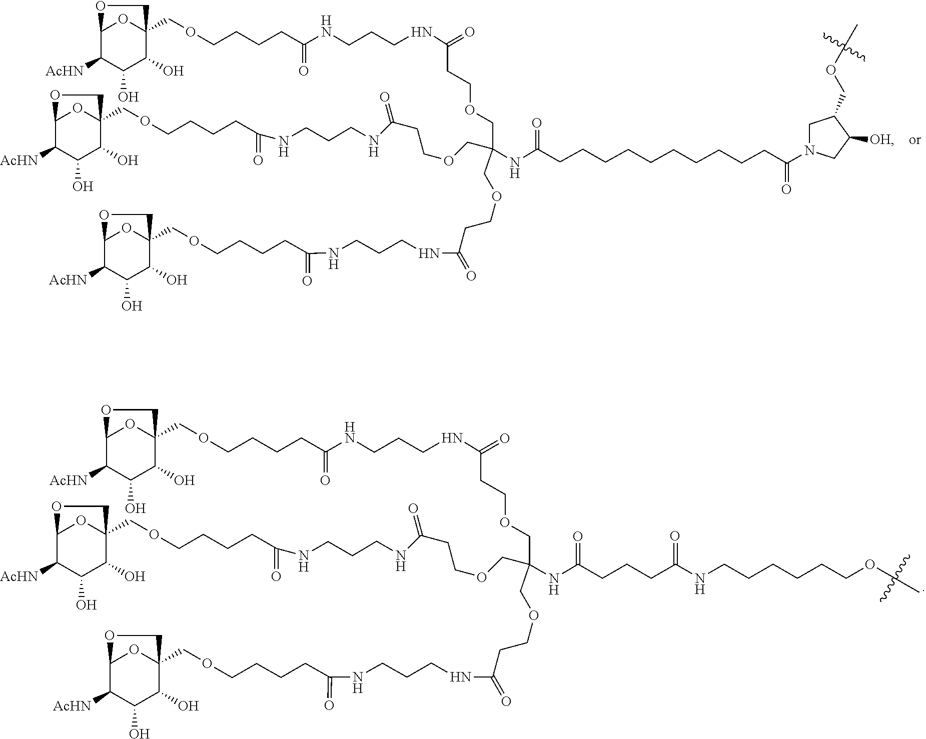

In certain embodiments, a tri-antennary ASPGR ligand is Mod 001, Mod083, Mod071, Mod153, or Mod155.

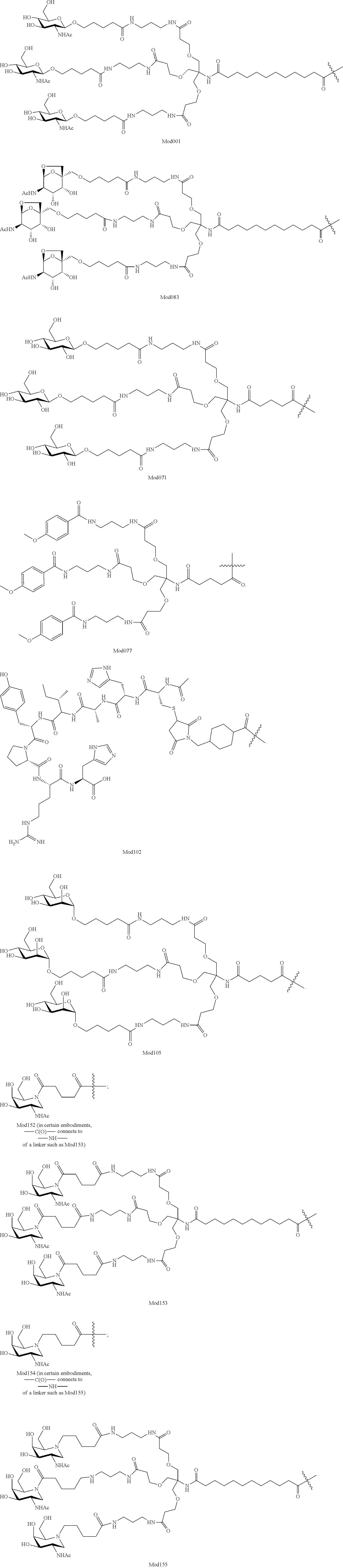

In some embodiments, an oligonucleotide comprises

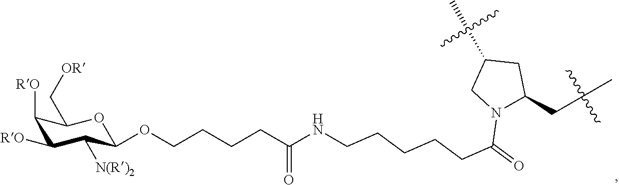

wherein each variable is independently as described herein. In some embodiments, each —OR′ is —OAc, and —N(R′)₂—NHAc. In some embodiments, an oligonucleotide comprises

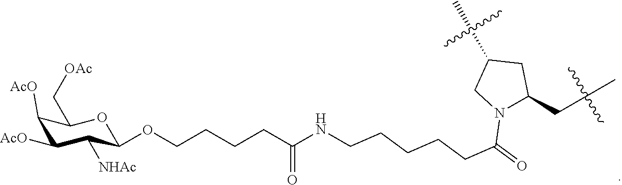

In some embodiments, each R′ is —H. In some embodiments, each —OR′ is —OH, and each —N(R′)₂ is —NHC(O)R. In some embodiments, each —OR′ is —OH, and each —N(R′)₂ is —NHAc. In some embodiments, an oligonucleotide comprises

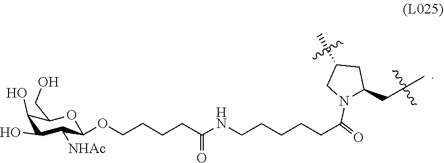

In some embodiments, the —CH₂— connection site is utilized as a C₅ connection site in a sugar. In some embodiments, the connection site on the ring is utilized as a C₃ connection site in a sugar. Such moieties may be introduced utilizing, e.g., phosphoramidites such as

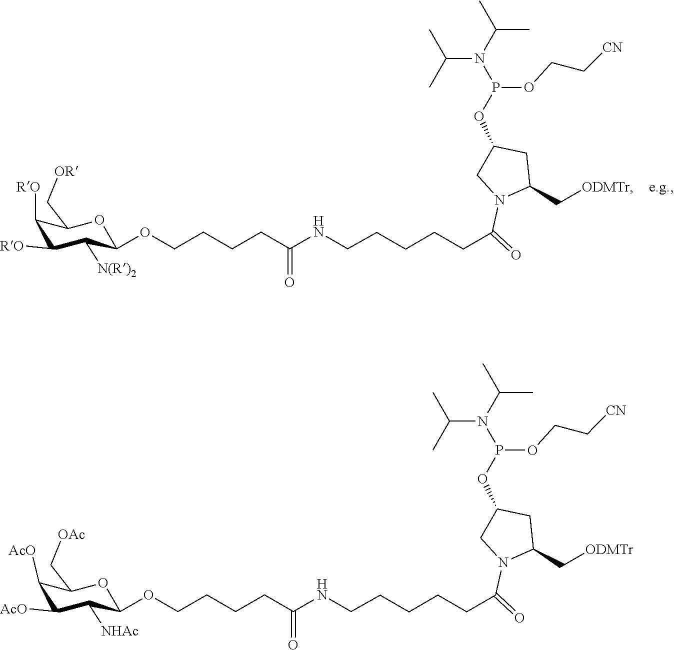

(those skilled in the art appreciate that one or more other groups, such as protection groups for —OH, —NH₂—, —N(i-Pr)₂, —OCH₂CH₂CN, etc., may be alternatively utilized, and protection groups can be removed under various suitable conditions, sometimes during oligonucleotide de-protection and/or cleavage steps). In some embodiments, an oligonucleotide comprises 2, 3 or more (e.g., 3 and no more than 3)

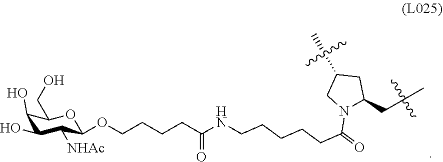

In some embodiments, an oligonucleotide comprises 2, 3 or more (e.g., 3 and no more than 3)

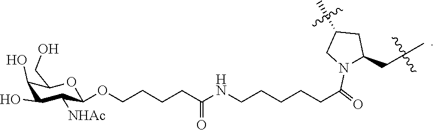

In some embodiments, copies of such moieties are linked by internucleotidic linkages, e.g., natural phosphate linkages, as described herein. In some embodiments, when at a 5′-end, a —CH₂— connection site is bonded to —OH. In some embodiments, an oligonucleotide comprises

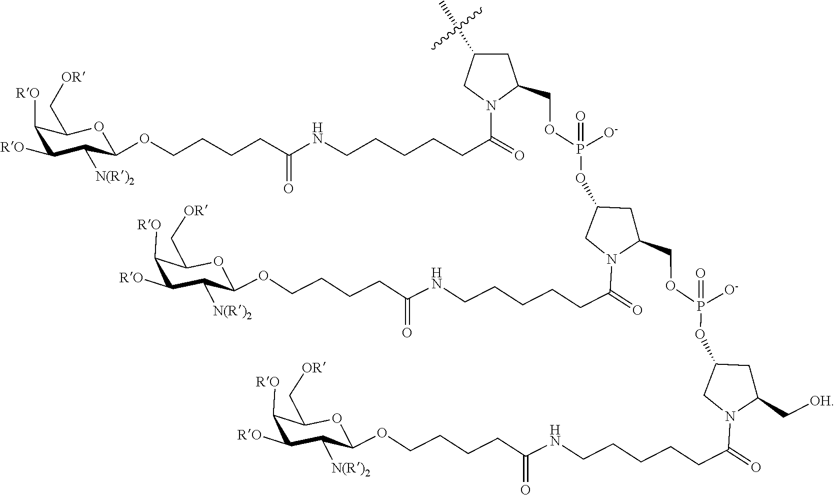

In some embodiments, each —OR′ is —OAc, and —N(R′)₂ is —NHAc. In some embodiments, an oligonucleotide comprises

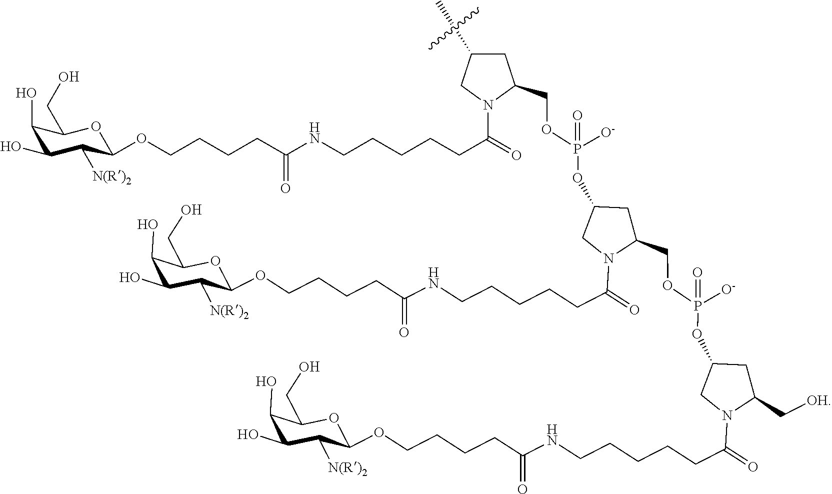

Among other things,

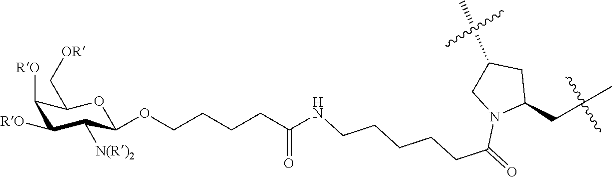

may be utilized to introduce

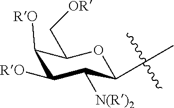

with comparable and/or better activities and/or properties. In some embodiments, it provides improved preparation efficiency and/or lower cost for the same number of

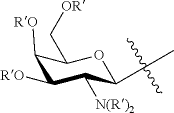

(e.g., when compared to Mod001).

In certain embodiments, an additional chemical moiety is a Mod group described herein, e.g., in Table 1.

In certain embodiments, an additional chemical moiety is Mod001. In certain embodiments, an additional chemical moiety is Mod083. In certain embodiments, an additional chemical moiety, e.g., a Mod group, is directly conjugated (e.g., without a linker) to the remainder of the ds oligonucleotide. In certain embodiments, an additional chemical moiety is conjugated via a linker to the remainder of the ds oligonucleotide. In certain embodiments, additional chemical moieties, e.g., Mod groups, may be directly connected, and/or via a linker, to nucleobases, sugars and/or internucleotidic linkages of ds oligonucleotides. In certain embodiments, Mod groups are connected, either directly or via a linker, to sugars. In certain embodiments, Mod groups are connected, either directly or via a linker, to 5′-end sugars. In certain embodiments, Mod groups are connected, either directly or via a linker, to 5′-end sugars via 5′ carbon. For examples, see various ds oligonucleotides in Table 1. In certain embodiments, Mod groups are connected, either directly or via a linker, to 3′-end sugars. In certain embodiments, Mod groups are connected, either directly or via a linker, to 3′-end sugars via 3′ carbon. In certain embodiments, Mod groups are connected, either directly or via a linker, to nucleobases. In certain embodiments, Mod groups are connected, either directly or via a linker, to internucleotidic linkages. In certain embodiments, provided oligonucleotides comprise Mod001 connected to 5′-end of oligonucleotide chains through L001.

As appreciated by those skilled in the art, an additional chemical moiety may be connected to a ds oligonucleotide chain at various locations, e.g., 5′-end, 3′-end, or a location in the middle (e.g., on a sugar, a base, an internucleotidic linkage, etc.). In certain embodiments, it is connected at a 5′-end. In certain embodiments, it is connected at a 3′-end. In certain embodiments, it is connected at a nucleotide in the middle. Certain additional chemical moieties (e.g., lipid moieties, targeting moieties, carbohydrate moieties), including but not limited to Mod012, Mod039, Mod062, Mod085, Mod086, and Mod094, and various linkers for connecting additional chemical moieties to ds oligonucleotide chains, including but not limited to L001, L003, L004, L008, L009, and L010, are described in WO 2017/062862, WO 2018/067973, WO 2017/160741, WO 2017/192679, WO 2017/210647, WO 2018/098264, WO 2018/022473, WO 2018/223056, WO 2018/223073, WO 2018/223081, WO 2018/237194, WO 2019/032607, WO2019032612, WO 2019/055951, WO 2019/075357, WO 2019/200185, WO 2019/217784, and/or WO 2019/032612, the additional chemical moieties and linkers of each of which are independently incorporated herein by reference, and can be utilized in accordance with the present disclosure. In certain embodiments, an additional chemical moiety is digoxigenin or biotin or a derivative thereof.

In certain embodiments, a ds oligonucleotide comprises a linker, e.g., L001 L004, L008, and/or an additional chemical moiety, e.g., Mod012, Mod039, Mod062, Mod085, Mod086, or Mod094. In certain embodiments, a linker, e.g., L001, L003, L004, L008, L009, L110, etc. is linked to a Mod, e.g., Mod012, Mod039, Mod062, Mod085, Mod086, Mod094, Mod152, Mod153, Mod154, Mod155 etc. L001: —NH—(CH₂)₆— linker (also known as a C₆ linker, C₆ amine linker or C₆ amino linker), connected to Mod, if any, through —NH—, and the 5′-end or 3′-end of the ds oligonucleotide chain through either a phosphate linkage (—O—P(O)(OH)—O—, which may exist as a salt form, and may be indicated as O or PO) or a phosphorothioate linkage (—O—P(O)(SH)—O—, which may exist as a salt form, and may be indicated as * if the phosphorothioate is not chirally controlled; or *S, S, or Sp, if the phosphorothioate is chirally controlled and has an Sp configuration, or *R, R, or Rp, if the phosphorothioate is chirally controlled and has an Rp configuration) as indicated at the —CH₂— connecting site. If no Mod is present, L001 is connected to —H through —NH—;
L003:

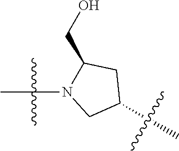

linker. In certain embodiments, it is connected to Mod, if any (if no Mod, —H), through its amino group, and the 5′-end or 3′-end of an oligonucleotide chain e.g., via a linkage (e.g., a phosphate linkage (0 or PO) or a phosphorothioate linkage (can be either not chirally controlled or chirally controlled (Sp or Rp))); L004: linker having the structure of —NH(CH₂)₄CH(CH₂OH)CH₂—, wherein —NH— is connected to Mod (through —C(O)—) or —H, and the —CH₂— connecting site is connected to an oligonucleotide chain (e.g., at the 3′-end) through a linkage, e.g., phosphodiester (—O—P(O)(OH)—O—, which may exist as a salt form, and may be indicated as O or PO), phosphorothioate (—O—P(O)(SH)—O—, which may exist as a salt form, and may be indicated as * if the phosphorothioate is not chirally controlled; or *S, S, or Sp, if the phosphorothioate is chirally controlled and has an Sp configuration, or *R, R, or Rp, if the phosphorothioate is chirally controlled and has an Rp configuration), or phosphorodithioate (—O—P(S)(SH)—O—, which may exist as a salt form, and may be indicated as PS2 or: or D) linkage. For example, an asterisk immediately preceding a L004 (e.g., *L004) indicates that the linkage is a phosphorothioate linkage, and the absence of an asterisk immediately preceding L004 indicates that the linkage is a phosphodiester linkage. For example, in an oligonucleotide which terminates in . . . mAL004, the linker L004 is connected (via the —CH₂— site) through a phosphodiester linkage to the 3′ position of the 3′-terminal sugar (which is 2′-OMe modified and connected to the nucleobase A), and the L004 linker is connected via —NH— to —H. Similarly, in one or more oligonucleotides, the L004 linker is connected (via the —CH₂— site) through the phosphodiester linkage to the 3′ position of the 3′-terminal sugar, and the L004 is connected via —NH— to, e.g., Mod012, Mod085, Mod086, etc.; L008: linker having the structure of —C(O)—(CH₂)₉—, wherein —C(O)— is connected to Mod (through —NH—) or —OH (if no Mod indicated), and the —CH₂— connecting site is connected to an oligonucleotide chain (e.g., at the 5′-end) through a linkage, e.g., phosphodiester (—O—P(O)(OH)—O—, which may exist as a salt form, and may be indicated as O or PO), phosphorothioate (—O—P(O)(SH)—O—, which may exist as a salt form, and may be indicated as * if the phosphorothioate is not chirally controlled; or *S, S, or Sp, if the phosphorothioate is chirally controlled and has an Sp configuration, or *R, R, or Rp, if the phosphorothioate is chirally controlled and has an Rp configuration), or phosphorodithioate (—O—P(S)(SH)—O—, which may exist as a salt form, and may be indicated as PS2 or: or D) linkage. For example, in an example oligonucleotide which has the sequence of 5′-L008 mN*mN*mN*mN*N*N*N*N*N*N*N*N*N*N*mN*mN*mN*mN-3′, and which has a Stereochemistry/Linkage of OXXXXXXXXX XXXXXXXX, wherein N is a base, wherein O is a natural phosphate internucleotidic linkage, and wherein X is a stereorandom phosphorothioate, L008 is connected to —OH through —C(O)—, and the 5′-end of an oligonucleotide chain through a phosphate linkage (indicated as “0” in “Stereochemistry/Linkage”); in another example oligonucleotide, which has the sequence of 5′-Mod062L008 mN*mN*mN*mN*N*N*N*N*N*N*N*N*N*N*mN*mN*mN*mN-3′, and which has a Stereochemistry/Linkage of OXXXXXXXXX XXXXXXXX, wherein N is a base, L008 is connected to Mod062 through —C(O)—, and the 5′-end of an oligonucleotide chain through a phosphate linkage (indicated as “O” in “Stereochemistry/Linkage”);
L009: —CH₂CH₂CH₂—. In certain embodiments, when L009 is present at the 5′-end of an oligonucleotide without a Mod, one end of L009 is connected to —OH and the other end connected to a 5′-carbon of the oligonucleotide chain e.g., via a linkage (e.g., a phosphate linkage (O or PO) or a phosphorothioate linkage (can be either not chirally controlled or chirally controlled (Sp or Rp))); L010:

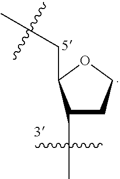

In certain embodiments, when LOW1 is present at the 5′-end of an oligonucleotide without a Mod, the 5′-carbon of L010 is connected to —OH and the 3′-carbon connected to a 5′-carbon of the oligonucleotide chain e.g., via a linkage (e.g., a phosphate linkage (O or PO) or a phosphorothioate or a phosphoryl guanidine linkage (can be either not chirally controlled or chirally controlled (Sp or Rp))); Mod012 (in certain embodiments, —C(O)— connects to —NH— of a linker such as L001, L004, L008, etc.):
L010 is utilized with n001R to form L010n001R, which has the structure of

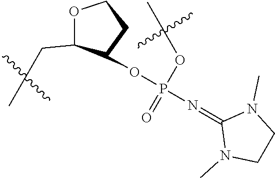

and wherein the configuration of linkage phosphorus is Rp. In some embodiments, multiple L010n001R may be utilized. For example, L023L010n001R^{L}010n001R^{L}010n001R, which has the following structure (which is bonded to the 5′-carbon at the 5′-end of the oligonucleotide chain, and each linkage phosphorus is independently Rp):

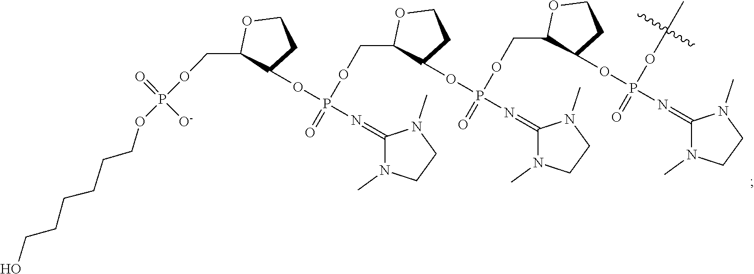

L023 is utilized with n001 to form L023n001, which has the structure of

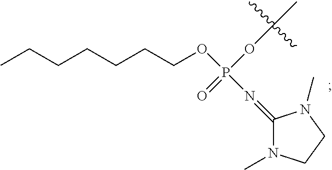 [SMILES: *OP(=O)(N=C1N(C)CCN1C)OCCCCCCC]

L023 is utilized with n009 to form L023n009, as in WV-42644 which has the structure of

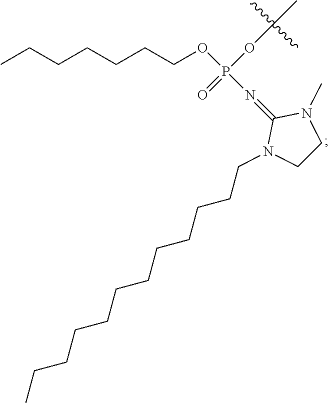 [SMILES: *OP(=O)(N=C1N(C)CCN1CCCCCCCCCCCC)OCCCCCCC]

In some embodiments, L023n001L009n001L009n001 may be utilized. For example, L023n001L009n001L009n001 as in WV-42643

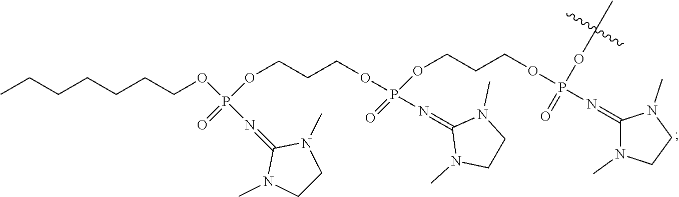 [SMILES: *OP(=O)(N=C1N(C)CCN1C)OCCCOP(=O)(N=C1N(C)CCN1C)OCCCOP(=O)(N=C1N(C)CCN1C)OCCCCCCC]

In some embodiments, L023n009L009n009 may be utilized. For example, as in WV-42646

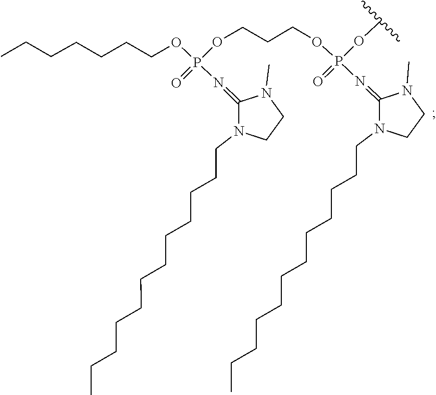 [SMILES: *OP(=O)(N=C1N(C)CCN1CCCCCCCCCCCC)OCCCOP(=O)(N=C1N(C)CCN1CCCCCCCCCCCC)OCCCCCCC]

In some embodiments, L023n009L009n009L009n009 may be utilized. For example, as in WV-42648

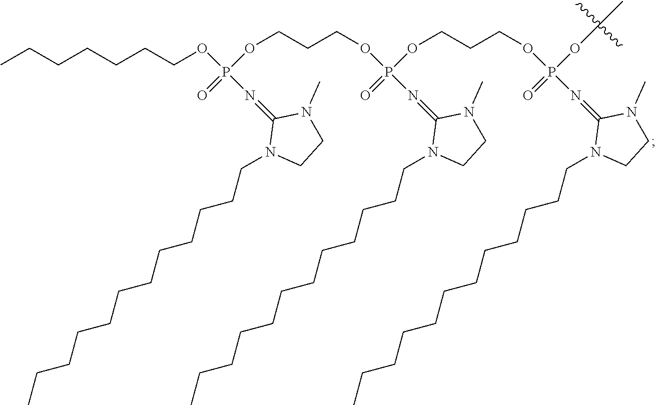 [SMILES: *OP(=O)(N=C1N(C)CCN1CCCCCCCCCCCC)OCCCOP(=O)(N=C1N(C)CCN1CCCCCCCCCCCC)OCCCOP(=O)(N=C1N(C)CCN1CCCCCCCCCCCC)OCCCCCCC]

In some embodiments L025 may be utilized; as in WV-41390,

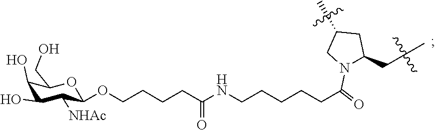

wherein the —CH₂— connection site is utilized as a C₅ connection site of a sugar (e.g., a DNA sugar) and is connected to another unit (e.g., 3′ of a sugar), and the connection site on the ring is utilized as a C₃ connection site and is connected to another unit (e.g., a 5′-carbon of a carbon), each of which is independently, e.g., via a linkage (e.g., a phosphate linkage (O or PO) or a phosphorothioate or a phosphoryl guanidine linkage (can be either not chirally controlled or chirally controlled (Sp or Rp))). When L025 is at a5′-end without any modifications, its —CH₂— connection site is bonded to —OH. For example, L025L025L025—in various oligonucleotides has the structure of

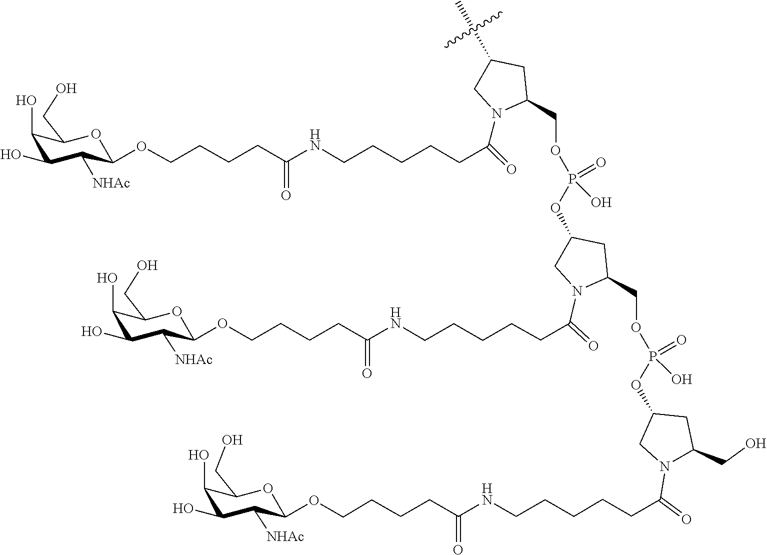

(may exist as various salt forms) and is connected to 5′-carbon of an oligonucleotide chain via a linkage as indicated (e.g., a phosphate linkage (O or PO) or a phosphorothioate or a phosphoryl guanidine linkage (can be either not chirally controlled or chirally controlled (Sp or Rp)));
In some embodiments L026 may be utilized; as in WV-44444,

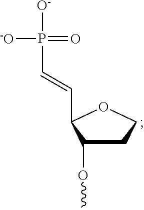

In some embodiments L027 may be utilized; as in WV-44445,

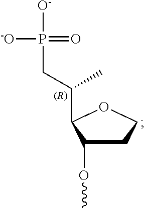

In some embodiments mU may be utilized; as in WV-42079,

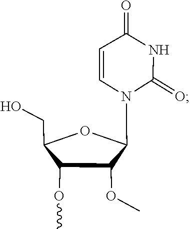

In some embodiments p[Rm5d5m]U may be utilized; as in SSR-0106183

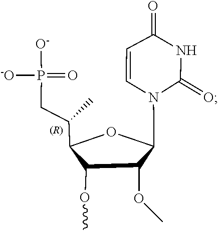

In some embodiments fU may be utilized; as in WV-44433,

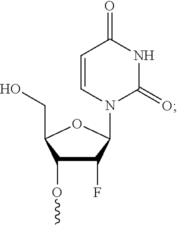

In some embodiments dT may be utilized; as in WV-44434,

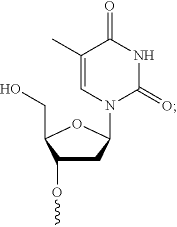

In some embodiments POdT or PO4-dT may be utilized; as in WV-44435,

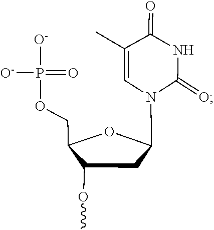

In some embodiments PO5MRdT may be utilized; as in WV-44436,

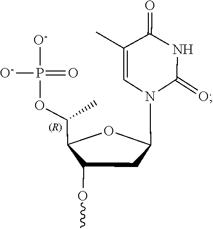

In some embodiments PO5MSdT may be utilized; as in WV-44437,

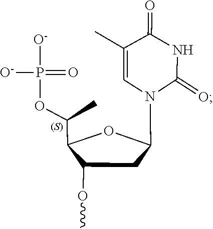

In some embodiments VPdT may be utilized; as in WV-44438,

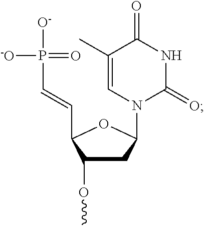

In some embodiments 5mvpdT may be utilized; as in WV-44439,

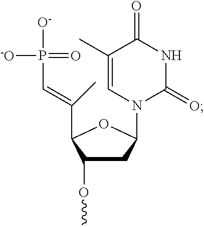

In some embodiments 5mrpdT may be utilized; as in WV-44440,

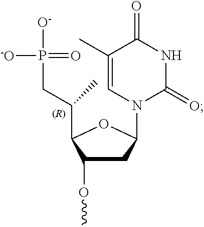

In some embodiments 5mspdT may be utilized; as in WV-44441,

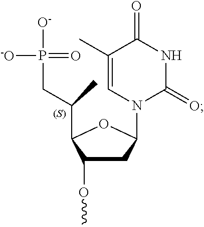

In some embodiments PNdT may be utilized; as in WV-44442,

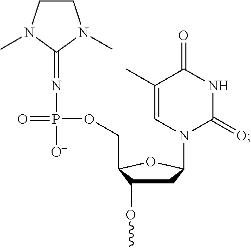

In some embodiments SPNdT may be utilized; as in WV-44443,

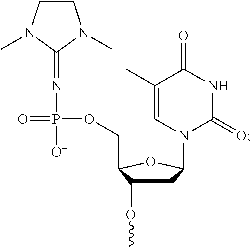

In some embodiments 5ptzdT may be utilized; as in WV-44446,

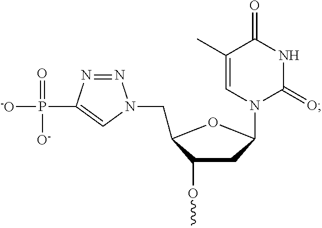

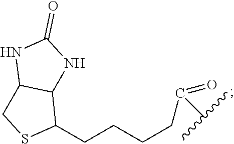 [SMILES: *C(=O)CCCCC1SCC2NC(=O)NC21]

Mod039 (in certain embodiments, —C(O)— connects to —NH— of a linker such as L001, L003, L004, L008, L009, L110, etc.):

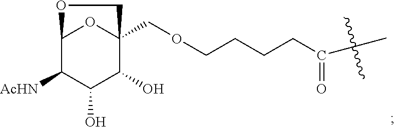 [SMILES: *C(=O)CCCCOC[C@@]12CO[C@@H](O1)[C@H](NC(C)=O)[C@@H](O)[C@H]2O]

Mod062 (in certain embodiments, —C(O)— connects to —NH— of a linker such as L001, L003, L004, L008, L009, L110, etc.):

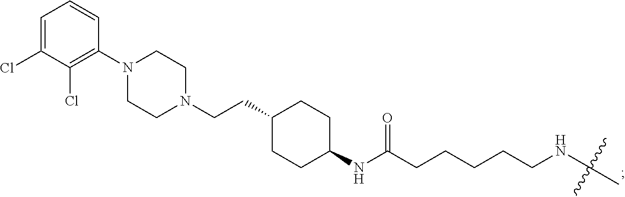 [SMILES: *NCCCCCC(=O)N[C@H]1CC[C@H](CCN2CCN(c3cccc(Cl)c3Cl)CC2)CC1]

Mod085 (in certain embodiments, —C(O)— connects to —NH— of a linker such as L001, L003, L004, L008, L009, L110, etc.):

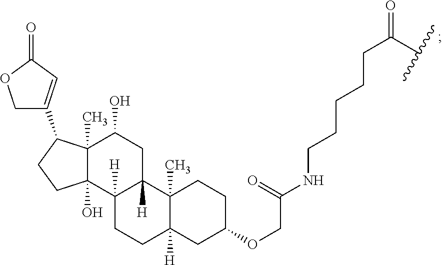

Mod086 (in certain embodiments, —C(O)— connects to —NH— of a linker such as L001, L003, L004, L008, L009, L110, etc.):

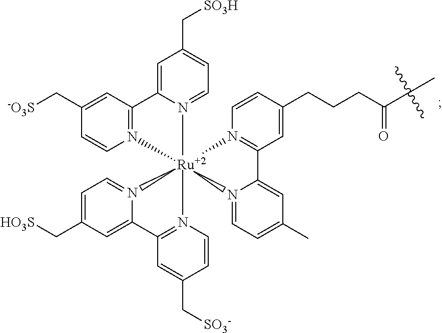

Mod094 (in certain embodiments, connects to an internucleotidic linkage, or to the 5′-end or 3′-end of an oligonucleotide via a linkage, e.g., a phosphate linkage, a phosphorothioate linkage (which is optionally chirally controlled), etc. For example, in an example oligonucleotide which has the sequence of 5′-mN*mN * mN*mN*N*N*N*N*N*N*N*N*N*N*mN*mN*mN*mNMod094-3′, and which has a Stereochemistry/Linkage of XXXXX XXXXX XXXXX XXO, wherein N is a base, Mod094 is connected to the 3′-end of the oligonucleotide chain (3′-carbon of the 3′-end sugar) through a phosphate group (which is not shown below and which may exist as a salt form; and which is indicated as “0” in “Stereochemistry/Linkage” ( . . . XXXXO))):

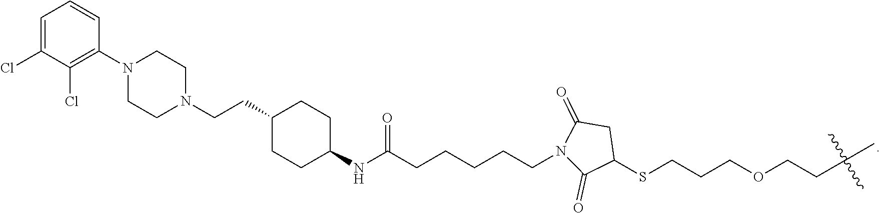 [SMILES: *CCOCCCSC1CC(=O)N(CCCCCC(=O)N[C@H]2CC[C@H](CCN3CCN(c4cccc(Cl)c4Cl)CC3)CC2)C1=O]

In certain embodiments, an additional chemical moiety is one described in WO 2012/030683. In certain embodiments, a provided ds oligonucleotide comprise a chemical structure (e.g., a linker, lipid, solubilizing group, and/or targeting ligand) described in WO 2012/030683.

In certain embodiments, a provided ds oligonucleotide comprises an additional chemical moiety and/or a modification (e.g., of nucleobase, sugar, internucleotidic linkage, etc.) described in: U.S. Pat. Nos. 5,688,941; 6,294,664; 6,320,017; 6,576,752; 5,258,506; 5,591,584; 4,958,013; 5,082,830; 5,118,802; 5,138,045; 6,783,931; 5,254,469; 5,414,077; 5,486,603; 5,112,963; 5,599,928; 6,900,297; 5,214,136; 5,109,124; 5,512,439; 4,667,025; 5,525,465; 5,514,785; 5,565,552; 5,541,313; 5,545,730; 4,835,263; 4,876,335; 5,578,717; 5,580,731; 5,451,463; 5,510,475; 4,904,582; 5,082,830; 4,762,779; 4,789,737; 4,824,941; 4,828,979; 5,595,726; 5,214,136; 5,245,022; 5,317,098; 5,371,241; 5,391,723; 4,948,882; 5,218,105; 5,112,963; 5,567,810; 5,574,142; 5,578,718; 5,608,046; 4,587,044; 4,605,735; 5,585,481; 5,292,873; 5,552,538; 5,512,667; 5,597,696; 5,599,923; 7,037,646; 5,587,371; 5,416,203; 5,262,536; 5,272,250; or 8,106,022.

In certain embodiments, an additional chemical moiety, e.g., a Mod, is connected via a linker. Various linkers are available in the art and may be utilized in accordance with the present disclosure, for example, those utilized for conjugation of various moieties with proteins (e.g., with antibodies to form antibody-drug conjugates), nucleic acids, etc. Certain useful linkers are described in U.S. Pat. No. 9,982,257, US 20170037399, US 20180216108, US 20180216107, U.S. Pat. No. 9,598,458, WO 2017/062862, WO 2018/067973, WO 2017/160741, WO 2017/192679, WO 2017/210647, WO 2018/098264, WO 2018/223056, WO 2018/237194, WO 2019/032607, WO 2019/055951, WO 2019/075357, WO 2019/200185, WO 2019/217784, and/or WO 2019/032612, the linker moieties of each which are independently incorporated herein by reference. In certain embodiments, a linker is, as non-limiting examples, L001, L004, L009 or LO10. In certain embodiments, an oligonucleotide comprises a linker, but not an additional chemical moiety other than the linker. In certain embodiments, a ds oligonucleotide comprises a linker, but not an additional chemical moiety other than the linker, wherein the linker is L001, L004, L009, or L010.

As demonstrated herein, provided technologies can provide high levels of activities and/or desired properties, in certain embodiments, without utilizing particular structural elements (e.g., modifications, linkage configurations and/or patterns, etc.) reported to be desired and/or necessary (e.g., those reported in WO 2019/219581), though certain such structural elements may be incorporated into ds oligonucleotides in combination with various other structural elements in accordance with the present disclosure. For example, in certain embodiments, ds oligonucleotides of the present disclosure have fewer nucleosides 3′ to a nucleoside opposite to a target nucleoside (e.g., a target adenosine), contain one or more phosphorothioate internucleotidic linkages at one or more positions where a phosphorothioate internucleotidic linkage was reportedly not favored or not allowed, contain one or more Sp phosphorothioate internucleotidic linkages at one or more positions where a Sp phosphorothioate internucleotidic linkage was reportedly not favored or not allowed, contain one or more Rp phosphorothioate internucleotidic linkages at one or more positions where a Rp phosphorothioate internucleotidic linkage was reportedly not favored or not allowed, and/or contain different modifications (e.g., internucleotidic linkage modifications, sugar modifications, etc.) and/or stereochemistry at one or more locations compared to those reportedly favorable or required for certain oligonucleotide properties and/or activities (e.g., presence of 2′-MOE, absence of phosphorothioate linkages at certain positions, absence of Sp phosphorothioate linkages at certain positions, and/or absence of Rp phosphorothioate linkages at certain positions were reportedly favorable or required for certain oligonucleotide properties and/or activities; as demonstrated herein, provided technologies can provide desired properties and/or high activities without utilizing 2′-MOE, without avoiding phosphorothioate linkages at one or more such certain positions, without avoiding Sp phosphorothioate linkages at one or more such certain positions, and/or without avoiding Rp phosphorothioate linkages at one or more such certain positions). Additionally or alternatively, provided ds oligonucleotides incorporates structural elements that were not previously recognized such as utilization of certain modifications (e.g., base modifications, sugar modifications (e.g., 2′-F), linkage modifications (e.g., non-negatively charged internucleotidic linkages), additional moieties, etc.) and levels, patterns, and combinations thereof.

For example, in certain embodiments, as described herein, provided d oligonucleotides contain no more than 5, 6, 7, 8, 9, 10, 11 or 12 nucleosides 3′ to a nucleoside opposite to a target nucleoside (e.g., a target adenosine).

Alternatively or additionally, as described herein (e.g., illustrated in certain Examples), for structural elements 3′ to a nucleoside opposite to a target nucleoside (e.g., a target adenosine), in certain embodiments, about 50%-100% (e.g., about or at least about 50%, 55%, 60%, 65%, 70%, 75%, 80%, 85%, 90%, 95%) of internucleotidic linkages 3′ to a nucleoside opposite to a target nucleoside (e.g., a target adenosine) are each independently a modified internucleotidic linkage, which is optionally chirally controlled. In certain embodiments, no more than 1, 2, or 3 internucleotidic linkages 3′ to a nucleoside opposite to a target nucleoside are natural phosphate linkages. In certain embodiments, no such internucleotidic linkage is natural phosphate linkages. In certain embodiments, no more than 1 such internucleotidic linkage is natural phosphate linkages. In certain embodiments, no more than 2 such internucleotidic linkages are natural phosphate linkages. In certain embodiments, no more than 3 such internucleotidic linkages are natural phosphate linkages. In certain embodiments, each modified internucleotidic linkage is independently a phosphorothioate or a non-negatively charged internucleotidic linkage (e.g., n001). In certain embodiments, each phosphorothioate internucleotidic linkage is chirally controlled. In certain embodiments, no more than 1, 2, or 3 internucleotidic linkages 3′ to a nucleoside opposite to a target nucleoside are Rp phosphorothioate internucleotidic linkage.

Alternatively or additionally, as described herein (e.g., illustrated in certain Examples), in certain embodiments, about 50%-100% (e.g., about or at least about 50%, 55%, 60%, 65%, 70%, 75%, 80%, 85%, 90%, or 95%) of internucleotidic linkages 5′ to a nucleoside opposite to a target nucleoside (e.g., a target adenosine) are each independently a modified internucleotidic linkage, which is optionally chirally controlled. In certain embodiments, no or no more than 1, 2, or 3 internucleotidic linkages 5′ to a nucleoside opposite to a target nucleoside (e.g., a target adenosine) are not modified internucleotidic linkages. In certain embodiments, no or no more than 1, 2, or 3 internucleotidic linkages 5′ to a nucleoside opposite to a target nucleoside (e.g., a target adenosine) are not phosphorothioate internucleotidic linkages. In certain embodiments, no or no more than 1, 2, or 3 internucleotidic linkages 5′ to a nucleoside opposite to a target nucleoside (e.g., a target adenosine) are not Sp phosphorothioate internucleotidic linkages. In certain embodiments, no more than 1, 2, or 3 internucleotidic linkages 5′ to a nucleoside opposite to a target nucleoside (e.g., a target adenosine) are natural phosphate linkages. In certain embodiments, no such internucleotidic linkage is natural phosphate linkages. In certain embodiments, no more than 1 such internucleotidic linkage is natural phosphate linkages. In certain embodiments, no more than 2 such internucleotidic linkages are natural phosphate linkages. In certain embodiments, no more than 3 such internucleotidic linkages are natural phosphate linkages. In certain embodiments, each modified internucleotidic linkage is independently a phosphorothioate or a non-negatively charged internucleotidic linkage (e.g., n001). In certain embodiments, there are no 2, 3, or 4 consecutive internucleotidic linkages 5′ to a nucleoside opposite to a target nucleoside, each of which is not a phosphorothioate internucleotidic linkage. In certain embodiments, there are no 2, 3, or 4 consecutive internucleotidic linkages 5′ to a nucleoside opposite to a target nucleoside, each of which is chirally controlled and is not a Sp phosphorothioate internucleotidic linkage. In certain embodiments, no or no more than 1, 2, 3, 4, or 5 internucleotidic linkages 5′ to a nucleoside opposite to a target nucleoside (e.g., a target adenosine) are Rp phosphorothioate internucleotidic linkage. In certain embodiments, at least about 1, 2, 3, 4, 5, 6, 7, 8, 9, or 10, or about 1, 2, 3, 4, 5, 6, 7, 8, 9, 10, 11, 12, 13, 14, 15, 16, 18, 19, 20, 21, 22, 23, 24, 25, 26, 27, 28, 29, 30, 31, or 32, or about 50%-100% (e.g., about or at least about 50%, 55%, 60%, 65%, 70%, 75%, 80%, 85%, 90%, or 95%) of internucleotidic linkages 5′ to a nucleoside opposite to a target nucleoside (e.g., a target adenosine) are each independently chirally controlled and a Sp internucleotidic linkage. In certain embodiments, at least about 1, 2, 3, 4, 5, 6, 7, 8, 9, or 10, or about 1, 2, 3, 4, 5, 6, 7, 8, 9, 10, 11, 12, 13, 14, 15, 16, 18, 19, 20, 21, 22, 23, 24, 25, 26, 27, 28, 29, 30, 31, or 32, or about 50%-100% (e.g., about or at least about 50%, 55%, 60%, 65%, 70%, 75%, 80%, 85%, 90%, or 95%) of phosphorothioate internucleotidic linkages 5′ to a nucleoside opposite to a target nucleoside (e.g., a target adenosine) are each independently chirally controlled and are Sp. In certain embodiments, each phosphorothioate internucleotidic linkages 5′ to a nucleoside opposite to a target nucleoside (e.g., a target adenosine) is chirally controlled. In certain embodiments, each phosphorothioate internucleotidic linkages 5′ to a nucleoside opposite to a target nucleoside (e.g., a target adenosine) is Sp.

### 6. Production of Oligonucleotides and Compositions

Various methods can be utilized for production of ds oligonucleotides and compositions and can be utilized in accordance with the present disclosure. For example, traditional phosphoramidite chemistry can be utilized to prepare stereorandom oligonucleotides and compositions, and certain reagents and chirally controlled technologies can be utilized to prepare chirally controlled oligonucleotide compositions, e.g., as described in U.S. Pat. No. 9,982,257, US 20170037399, US 20180216108, US 20180216107, U.S. Pat. No. 9,598,458, WO 2017/062862, WO 2018/067973, WO 2017/160741, WO 2017/192679, WO 2017/210647, WO 2018/098264, WO 2018/223056, WO 2018/237194, WO 2019/032607, WO 2019/055951, WO 2019/075357, WO 2019/200185, WO 2019/217784, and/or WO 2019/032612, the reagents and methods of each of which is incorporated herein by reference.

In certain embodiments, chirally controlled/stereoselective preparation of ds oligonucleotides and compositions thereof comprise utilization of a chiral auxiliary, e.g., as part of monomeric phosphoramidites. Examples of such chiral auxiliary reagents and phosphoramidites are described in U.S. Pat. No. 9,982,257, US 20170037399, US 20180216108, US 20180216107, U.S. Pat. No. 9,598,458, WO 2017/062862, WO 2018/067973, WO 2017/160741, WO 2017/192679, WO 2017/210647, WO 2018/098264, WO 2018/223056, WO 2018/237194, WO 2019/032607, WO 2019/055951, WO 2019/075357, WO 2019/200185, WO 2019/217784, and/or WO 2019/032612, the chiral auxiliary reagents and phosphoramidites of each of which are independently incorporated herein by reference. In certain embodiments, a chiral auxiliary is a chiral auxiliary described in any of: WO 2018/022473, WO 2018/098264, WO 2018/223056, WO 2018/223073, WO 2018/223081, WO 2018/237194, WO 2019/032607, WO 2019/055951, WO 2019/075357, WO 2019/200185, WO 2019/217784, and/or WO 2019/032612, the chiral auxiliaries of each of which are independently incorporated herein by reference.

In certain embodiments, chirally controlled preparation technologies, including oligonucleotide synthesis cycles, reagents and conditions are described in U.S. Pat. No. 9,982,257, US 20170037399, US 20180216108, US 20180216107, U.S. Pat. No. 9,598,458, WO 2017/062862, WO 2018/067973, WO 2017/160741, WO 2017/192679, WO 2017/210647, and/WO 2018/098264, WO 2018/022473, WO 2018/223056, WO 2018/223073, WO 2018/223081, WO 2018/237194, WO 2019/032607, WO2019032612, WO 2019/055951, WO 2019/075357, WO 2019/200185, WO 2019/217784, and/or WO 2019/032612, WO 2018/223056, WO 2018/223073, WO 2018/223081, WO 2018/237194, WO 2019/032607, WO 2019/055951, WO 2019/075357, WO 2019/200185, WO 2019/217784, and/or WO 2019/032612, the oligonucleotide synthesis methods, cycles, reagents and conditions of each of which are independently incorporated herein by reference.

Once synthesized, provided ds oligonucleotides and compositions are typically further purified. Suitable purification technologies are widely known and practiced by those skilled in the art, including but not limited to those described in U.S. Pat. No. 9,982,257, US 20170037399, US 20180216108, US 20180216107, U.S. Pat. No. 9,598,458, WO 2017/062862, WO 2018/067973, WO 2017/160741, WO 2017/192679, WO 2017/210647, WO 2018/098264, WO 2018/223056, WO 2018/237194, WO 2019/032607, WO 2019/055951, WO 2019/075357, WO 2019/200185, WO 2019/217784, and/or WO 2019/032612, the purification technologies of each of which are independently incorporated herein by reference.

In certain embodiments, a cycle comprises or consists of coupling, capping, modification and deblocking. In certain embodiments, a cycle comprises or consists of coupling, capping, modification, capping and deblocking. These steps are typically performed in the order they are listed, but in certain embodiments, as appreciated by those skilled in the art, the order of certain steps, e.g., capping and modification, may be altered. If desired, one or more steps may be repeated to improve conversion, yield and/or purity as those skilled in the art often perform in syntheses. For example, in certain embodiments, coupling may be repeated; in certain embodiments, modification (e.g., oxidation to install ═O, sulfurization to install=S, etc.) may be repeated; in certain embodiments, coupling is repeated after modification which can convert a P(III) linkage to a P(V) linkage which can be more stable under certain circumstances, and coupling is routinely followed by modification to convert newly formed P(III) linkages to P(V) linkages. In certain embodiments, when steps are repeated, different conditions may be employed (e.g., concentration, temperature, reagent, time, etc.).

Technologies for formulating provided ds oligonucleotides and/or preparing pharmaceutical compositions, e.g., for administration to subjects via various routes, are readily available in the art and can be utilized in accordance with the present disclosure, e.g., those described in U.S. Pat. No. 9,982,257, US 20170037399, US 20180216108, US 20180216107, U.S. Pat. No. 9,598,458, WO 2017/062862, WO 2018/067973, WO 2017/160741, WO 2017/192679, WO 2017/210647, WO 2018/098264, WO 2018/223056, or WO 2018/237194 and references cited therein.

Technologies for formulating provided ds oligonucleotides and/or preparing pharmaceutical compositions, e.g., for administration to subjects via various routes, are readily available in the art and can be utilized in accordance with the present disclosure, e.g., those described in U.S. Pat. No. 9,982,257, US 20170037399, US 20180216108, US 20180216107, U.S. Pat. No. 9,598,458, WO 2017/062862, WO 2018/067973, WO 2017/160741, WO 2017/192679, WO 2017/210647, WO 2018/098264, WO 2018/223056, or WO 2018/237194 and references cited therein.

In certain embodiments, a useful chiral auxiliary has the structure of

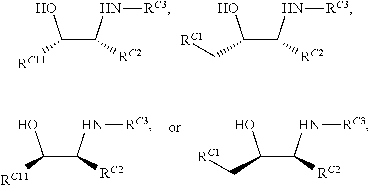

or a salt thereof, wherein R^{C11} is -L^{C1}-R^{C1}, L^{C1} is optionally substituted —CH₂—. R^{C1} is R, —Si(R)₃, —SO₂R or an electron-withdrawing group, and R^{C2} and R^{C3} are taken together with their intervening atoms to form an optionally substituted 3-10 membered saturated ring having, in addition to the nitrogen atom, 0-2 heteroatoms. In certain embodiments, a useful chiral auxiliary has the structure of

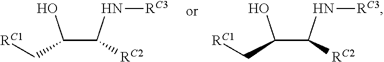

wherein R^{C1} is R, —Si(R)₃ or —SO₂R, and R^{C2} and R^{C3} are taken together with their intervening atoms to form an optionally substituted 3-7 membered saturated ring having, in addition to the nitrogen atom, 0-2 heteroatoms. is a formed ring is an optionally substituted 5-membered ring. In certain embodiments, a useful chiral auxiliary has the structure of

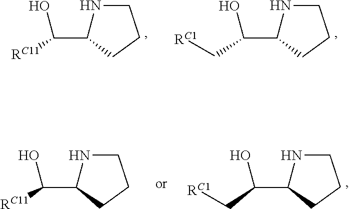

or a salt thereof. In certain embodiments, a useful chiral auxiliary has the structure of

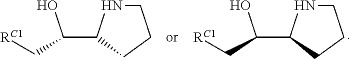

In certain embodiments, a useful chiral auxiliary is a DPSE chiral auxiliary. In certain embodiments, purity or stereochemical purity of a chiral auxiliary is at least 85%, 90%, 95%, 96%, 97%, 98%, or 99%. In certain embodiments, it is at least 85%. In certain embodiments, it is at least 90%. In certain embodiments, it is at least 95%. In certain embodiments, it is at least 96%. In certain embodiments, it is at least 97%. In certain embodiments, it is at least 98%. In certain embodiments, it is at least 99%.

In certain embodiments, L^{C1} is —CH₂—. In certain embodiments, L^{C1} is substituted —CH₂—. In certain embodiments, L^{C1} is monosubstituted —CH₂—.

In certain embodiments, R^{C1} is R. In certain embodiments, R^{C1} is optionally substituted phenyl. In certain embodiments, R^{C1} is —SiR₃. In certain embodiments, R^{C1} is —SiPh₂Me. In certain embodiments, R^{C1} is —SO₂R. In certain embodiments, R is not hydrogen. In certain embodiments, R is optionally substituted phenyl. In certain embodiments, R is phenyl. In certain embodiments, R is optionally substituted C₁₋₆ aliphatic. In certain embodiments, R is C₁₋₆ alkyl. In certain embodiments, R is methyl. In certain embodiments, R is t-butyl.

In certain embodiments, R^{C1} is an electron-withdrawing group, such as —C(O)R, —OP(O)(OR)₂, —OP(O)(R)₂, —P(O)(R)₂, —S(O)R, —S(O)₂R, etc. In certain embodiments, chiral auxiliaries comprising electron-withdrawing group R^{C1} groups are particularly useful for preparing chirally controlled non-negatively charged internucleotidic linkages and/or chirally controlled internucleotidic linkages bonded to natural RNA sugar.

In certain embodiments, R^{C2} and R^{C3} are taken together with their intervening atoms to form an optionally substituted 3-10 (e.g., 3, 4, 5, 6, 7, 8, 9, or 10) membered saturated ring having no heteroatoms in addition to the nitrogen atom. In certain embodiments, R^{C2} and R^{C3} are taken together with their intervening atoms to form an optionally substituted 5-membered saturated ring having no heteroatoms in addition to the nitrogen atom.

In certain embodiments, the present disclosure provides useful reagents for preparation of ds oligonucleotides and compositions thereof. In certain embodiments, phosphoramidites comprise nucleosides, nucleobases and sugars as described herein. In certain embodiments, nucleobases and sugars are properly protected for oligonucleotide synthesis as those skilled in the art will appreciate. In certain embodiments, a phosphoramidite has the structure of R^{NS}—P(OR)N(R)₂, wherein R^{NS} is an optionally protected nucleoside moiety. In certain embodiments, a phosphoramidite has the structure of R^{NS}—P(OCH₂CH₂CN)N(i-Pr)₂. In certain embodiments, a phosphoramidite comprises a nucleobase which is or comprises Ring BA, wherein Ring BA has the structure of BA-I, BA-I-a, BA-I-b, BA-II, BA-II-a, BA-II-b, BA-III, BA-III-a, BA-III-b, BA-IV, BA-IV-a, BA-IV-b, BA-V, BA-V-a, BA-V-b, or BA-VI, or a tautomer of Ring BA, wherein the nucleobase is optionally substituted or protected. In certain embodiments, a phosphoramidite comprises a chiral auxiliary moiety, wherein the phosphorus is bonded to an oxygen and a nitrogen atom of the chiral auxiliary moiety. In certain embodiments, a phosphoramidite has the structure of

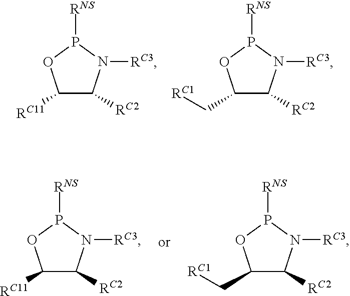

or a salt thereof, wherein R^{NS} is a protected nucleoside moiety (e.g., 5′-OH and/or nucleobases suitably protected for oligonucleotide synthesis), and each other variable is independently as described herein. In certain embodiments, a phosphoramidite has the structure of

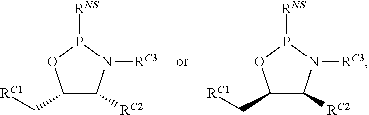

wherein R^{NS} is a protected nucleoside moiety (e.g., 5′—OH and/or nucleobases suitably protected for oligonucleotide synthesis), R^{C1} is R, —Si(R)₃ or —SO₂R, and R^{C2} and R^{C3} are taken together with their intervening atoms to form an optionally substituted 3-7 membered saturated ring having, in addition to the nitrogen atom, 0-2 heteroatoms, wherein the coupling forms an internucleotidic linkage. In certain embodiments, 5′—OH of R^{N}S is protected. In certain embodiments, 5′—OH of R^{NS} is protected as —ODMTr. In certain embodiments, R^{NS} is bonded to phosphorus through its 3′—O—. In certain embodiments, a formed ring by R^{C2} and R^{C3} is an optionally substituted 5-membered ring. In certain embodiments, a phosphoramidite has the structure of

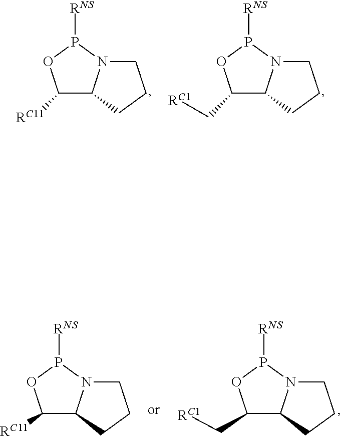

or a salt thereof. In certain embodiments, a phosphoramidite has the structure of

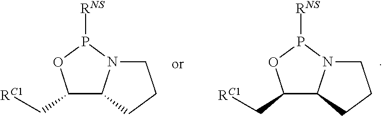

In certain embodiments, purity or stereochemical purity of a phosphoramidite is at least 85%, 90%, 95%, 96%, 97%, 98%, or 99%. In certain embodiments, it is at least 85%. In certain embodiments, it is at least 90%. In certain embodiments, it is at least 95%.

In certain embodiments, the present disclosure provides a method for preparing an oligonucleotide or composition, comprising coupling a free —OH, e.g., a free 5′—OH, of an oligonucleotide or a nucleoside with a phosphoramidite as described herein.

In certain embodiments, the present disclosure provides an oligonucleotide, wherein the oligonucleotide comprises one or more modified internucleotidic linkages each independently having the structure of —O⁵—P^{L}(W)(R^{CA})—O³—, wherein:
P^{L} is P, or P(═W);
W is O, S, or W^{N};
W^{N} is ═N—C(—N(R)₂═N⁺(R¹)₂Q⁻;
Q⁻ is an anion;
R^{CA} is or comprises an optionally capped chiral auxiliary moiety,
O⁵ is an oxygen bonded to a 5′-carbon of a sugar, and
O³ is an oxygen bonded to a 3′-carbon of a sugar.

In certain embodiments, a modified internucleotidic linkage is optionally chirally controlled. In certain embodiments, a modified internucleotidic linkage is optionally chirally controlled.

In certain embodiments, a provided methods comprising removing R^{CA} from such a modified internucleotidic linkages. In certain embodiments, after removal, bonding to R^{CA} is replaced with —OH. In certain embodiments, after removal, bonding to R^{CA} is replaced with ═O, and bonding to W^{N} is replaced with —N═C(N(R¹)₂)₂.

In certain embodiments, P^{L} is P═S, and when R^{CA} is removed, such an internucleotidic linkage is converted into a phosphorothioate internucleotidic linkage.

In certain embodiments, P^{L} is P═W^{N}, and when R^{CA} is removed, such an internucleotidic linkage is converted into an internucleotidic linkage having the structure of

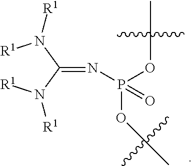

In certain embodiments, an internucleotidic linkage having the structure of

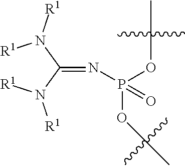

has the structure of

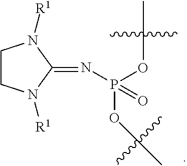

In certain embodiments, an internucleotidic linkage having the structure of

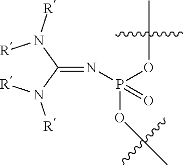

has the structure of

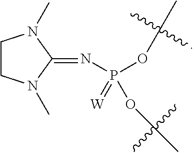

In certain embodiments, P^{L} is P (e.g., in newly formed internucleotidic linkage from coupling of a phosphoramidite with a 5′—OH). In certain embodiments, W is O or S. In certain embodiments, W is S (e.g., after sulfurization). In certain embodiments, W is O (e.g., after oxidation). In certain embodiments, certain non-negatively charged internucleotidic linkages or neutral internucleotidic linkages may be prepared by reacting a P(III) phosphite triester internucleotidic linkage with azido imidazolinium salts (e.g., compounds comprising

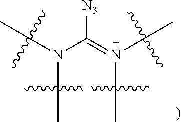

under suitable conditions. In certain embodiments, an azido imidazolinium salt is a salt of PF₆⁻. In certain embodiments, an azido imidazolinium salt is a slat of

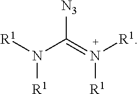

In certain embodiments, an azido imidazolinium salt is 2-azido-1,3-dimethylimidazolinium hexafluorophosphate.

As appreciated by those skilled in the art, Q⁻ can be various suitable anion present in a system (e.g., in oligonucleotide synthesis), and may vary during oligonucleotide preparation processes depending on cycles, process stages, reagents, solvents, etc. In certain embodiments, Q⁻ is PF₆⁻.

In certain embodiments, R^{CA} is

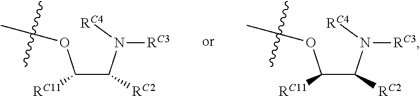

wherein R^{C4} is —H or —C(O)R′, and each other variable is independently as described herein. In certain embodiments, R^{CA} is

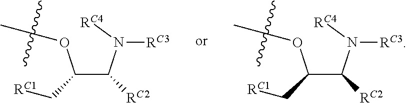

wherein R^{C1} is R, —Si(R)₃ or —SO₂R, R^{C2} and R^{C3} are taken together with their intervening atoms to form an optionally substituted 3-7 membered saturated ring having, in addition to the nitrogen atom, 0-2 heteroatoms, R^{C4} is —H or —C(O)R′. In certain embodiments, R^{C4} is —H. In certain embodiments, R^{C4} is —C(O)CH₃. In certain embodiments, R^{C2} and R^{C3} are taken together to form an optionally substituted 5-membered ring.

In certain embodiments, R^{C4} is —H (e.g., in n newly formed internucleotidic linkage from coupling of a phosphoramidite with a 5′—OH). In certain embodiments, R^{C4} is —C(O)R (e.g., after capping of the amine). In certain embodiments, R is methyl.

In certain embodiments, each chirally controlled phosphorothioate internucleotidic linkage is independently converted from —O⁵—P^{L}(W)(R^{CA})—O³—.

### 8. Characterization and Assessment

In certain embodiments, properties and/or activities of dsRNAi oligonucleotides and compositions thereof can be characterized and/or assessed using various technologies available to those skilled in the art, e.g., biochemical assays, cell based assays, animal models, clinical trials, etc.

In certain embodiments, a method of identifying and/or characterizing an oligonucleotide composition, e.g., a dsRNAi oligonucleotide composition, comprises steps of: providing at least one composition comprising a plurality of oligonucleotides; and assessing delivery relative to a reference composition.

In certain embodiments, the present disclosure provides a method of identifying and/or characterizing a ds oligonucleotide composition, e.g., a dsRNAi oligonucleotide composition, comprises steps of: providing at least one composition comprising a plurality of ds oligonucleotides; and assessing cellular uptake relative to a reference composition.

In certain embodiments, the present disclosure provides a method of identifying and/or characterizing a ds oligonucleotide composition, e.g., a dsRNAi oligonucleotide composition, comprises steps of: providing at least one composition comprising a plurality of ds oligonucleotides; and assessing reduction of transcripts of a target gene and/or a product encoded thereby relative to a reference composition.

In certain embodiments, the present disclosure provides a method of identifying and/or characterizing a ds oligonucleotide composition, e.g., a dsRNAi oligonucleotide composition, comprises steps of: providing at least one composition comprising a plurality of ds oligonucleotides; and assessing reduction of tau levels, its aggregation and/or spreading relative to a reference composition.

In certain embodiments, properties and/or activities of ds oligonucleotides, e.g., dsRNAi oligonucleotides, and compositions thereof are compared to reference ds oligonucleotides and compositions thereof, respectively.

In certain embodiments, a reference ds oligonucleotide composition is a stereorandom ds oligonucleotide composition. In certain embodiments, a reference ds oligonucleotide composition is a stereorandom composition of ds oligonucleotides of which all internucleotidic linkages are phosphorothioate. In certain embodiments, a reference ds oligonucleotide composition is a ds DNA oligonucleotide composition with all phosphate linkages. In certain embodiments, a reference ds oligonucleotide composition is otherwise identical to a provided chirally controlled ds oligonucleotide composition except that it is not chirally controlled. In certain embodiments, a reference ds oligonucleotide composition is otherwise identical to a provided chirally controlled oligonucleotide composition except that it has a different pattern of stereochemistry. In certain embodiments, a reference ds oligonucleotide composition is similar to a provided ds oligonucleotide composition except that it has a different modification of one or more sugar, base, and/or internucleotidic linkage, or pattern of modifications. In certain embodiments, a ds oligonucleotide composition is stereorandom and a reference ds oligonucleotide composition is also stereorandom, but they differ in regard to sugar and/or base modification(s) or patterns thereof.

In certain embodiments, a reference composition is a composition of ds oligonucleotides having the same base sequence and the same chemical modifications. In certain embodiments, a reference composition is a composition of ds oligonucleotides having the same base sequence and the same pattern of chemical modifications. In certain embodiments, a reference composition is a non-chirally controlled (or stereorandom) composition of ds oligonucleotides having the same base sequence and chemical modifications. In certain embodiments, a reference composition is a non-chirally controlled (or stereorandom) composition of ds oligonucleotides of the same constitution but is otherwise identical to a provided chirally controlled ds oligonucleotide composition.

In certain embodiments, a reference ds oligonucleotide composition is of ds oligonucleotides having a different base sequence. In certain embodiments, a reference ds oligonucleotide composition is of ds oligonucleotides that do not target RNAi (e.g., as negative control for certain assays).

In certain embodiments, a reference composition is a composition of ds oligonucleotides having the same base sequence but different chemical modifications, including but not limited to chemical modifications described herein. In certain embodiments, a reference composition is a composition of ds oligonucleotides having the same base sequence but different patterns of internucleotidic linkages and/or stereochemistry of internucleotidic linkages and/or chemical modifications.

Various methods are known in the art for detection of gene products, the expression, level and/or activity of which may be altered after introduction or administration of a provided ds oligonucleotide. For example, transcripts and their knockdown can be detected and quantified with qPCR, and protein levels can be determined via Western blot.

In certain embodiments, assessment of efficacy of ds oligonucleotides can be performed in biochemical assays or in vitro in cells. In certain embodiments, dsRNAi oligonucleotides can be introduced to cells via various methods available to those skilled in the art, e.g., gymnotic delivery, transfection, lipofection, etc.

In certain embodiments, the efficacy of a putative dsRNAi oligonucleotide can be tested in vitro.

In certain embodiments, the efficacy of a putative dsRNAi oligonucleotide can be tested in vitro using any known method of testing the expression, level and/or activity of a gene or gene product thereof.

In certain embodiments, dsRNAi soluble aggregates can be observed by immunoblotting.

In certain embodiments, a dsRNAi oligonucleotide is tested in a cell or animal model of a disease.

In certain embodiments, an animal model administered a dsRNAi oligonucleotide can be evaluated for safety and/or efficacy.

In certain embodiments, the effect(s) of administration of a ds oligonucleotide to an animal can be evaluated, including any effects on behavior, inflammation, and toxicity. In certain embodiments, following dosing, animals can be observed for signs of toxicity including trouble grooming, lack of food consumption, and any other signs of lethargy. In certain embodiments, in a mouse model, following administration of a dsRNAi oligonucleotide, the animals can be monitored for timing of onset of a rear paw clasping phenotype.

In certain embodiments, following administration of a dsRNAi oligonucleotide to an animal, the animal can be sacrificed and analysis of tissues or cells can be performed to determine changes in RNAi activity, or other biochemical or other changes. In certain embodiments, following necropsy, liver, heart, lung, kidney, and spleen can be collected, fixed, and processed for histopathological evaluation (standard light microscopic examination of hematoxylin and eosin-stained tissue slides).

In certain embodiments, following administration of a dsRNAi oligonucleotide to an animal, behavioral changes can be monitored or assessed. In certain embodiments, such an assessment can be performed using a technique described in the scientific literature.

Various effects of testing in animals described herein can also be monitored in human subjects or patients following administration of a dsRNAi oligonucleotide.

In addition, the efficacy of a dsRNAi oligonucleotide in a human subject can be measured by evaluating, after administration of the oligonucleotide, any of various parameters known in the art, including but not limited to a reduction in a symptom, or a decrease in the rate of worsening or onset of a symptom of a disease.

In certain embodiments, following human treatment with a ds oligonucleotide, or contacting a cell or tissue in vitro with an oligonucleotide, cells and/or tissues are collected for analysis.

In certain embodiments, in various cells and/or tissues, target nucleic acid levels can be quantitated by methods available in the art, many of which can be accomplished with commercially available kits and materials. Such methods include, e.g., Northern blot analysis, competitive polymerase chain reaction (PCR), quantitative real-time PCR, etc. RNA analysis can be performed on total cellular RNA or poly(A)+ mRNA. Probes and primers are designed to hybridize to a nucleic acid to be detected. Methods for designing real-time PCR probes and primers are well known and widely practiced in the art. For example, to detect and quantify RNAi RNA, an example method comprises isolation of total RNA (e.g., including mRNA) from a cell or animal treated with an oligonucleotide or a composition and subjecting the RNA to reverse transcription and/or quantitative real-time PCR, for example, as described herein, or in: Moon et al. 2012 Cell Metab. 15: 240-246.

In certain embodiments, protein levels can be evaluated or quantitated in various methods known in the art, e.g., enzyme-linked immunosorbent assay (ELISA), Western blot analysis (immunoblotting), immunocytochemistry, fluorescence-activated cell sorting (FACS), immuno-histochemistry, immunoprecipitation, protein activity assays (for example, caspase activity assays), and quantitative protein assays. Antibodies useful for the detection of mouse, rat, monkey, and human proteins are commercially available or can be generated if needed. For example, various RNAi antibodies have been reported.

Various technologies are available and/or known in the art for detecting levels of ds oligonucleotides or other nucleic acids. Such technologies are useful for detecting dsRNAi oligonucleotides when administered to assess, e.g., delivery, cell uptake, stability, distribution, etc.

In certain embodiments, selection criteria are used to evaluate the data resulting from various assays and to select particularly desirable ds oligonucleotides, e.g., desirable dsRNAi oligonucleotides, with certain properties and activities. In certain embodiments, selection criteria include an IC₅₀ of less than about 10 nM, less than about 5 nM or less than about 1 nM. In certain embodiments, selection criteria for a stability assay include at least 50% stability [at least 50% f an oligonucleotide is still remaining and/or detectable] at Day 1. In certain embodiments, selection criteria for a stability assay include at least 50% stability at Day 2. In certain embodiments, selection criteria for a stability assay include at least 50% stability at Day 3. In certain embodiments, selection criteria for a stability assay include at least 50% stability at Day 4. In certain embodiments, selection criteria for a stability assay include at least 50% stability at Day 5. In certain embodiments, selection criteria for a stability assay include at least 80% [at least 80% f the oligonucleotide remains] at Day 5.

In certain embodiments, efficacy of a dsRNAi oligonucleotide is assessed directly or indirectly by monitoring, measuring or detecting a change in a condition, disorder or disease or a biological pathway.

In certain embodiments, efficacy of a dsRNAi oligonucleotide is assessed directly or indirectly by monitoring, measuring or detecting a change in a response to be affected by knockdown.

In certain embodiments, a provided ds oligonucleotide (e.g., a dsRNAi oligonucleotide) can by analyzed by a sequence analysis to determine what other genes (e.g., genes which are not a target gene) have a sequence which is complementary to the base sequence of the provided ds oligonucleotide (e.g., the dsRNAi oligonucleotide) or which have 0, 1, 2 or more mismatches from the base sequence of the provided ds oligonucleotide (e.g., the dsRNAi oligonucleotide). Knockdown, if any, by the ds oligonucleotide of these potential off-targets can be determined to evaluate potential off-target effects of a ds oligonucleotide (e.g., a dsRNAi oligonucleotide). In certain embodiments, an off-target effect is also termed an unintended effect and/or related to hybridization to a bystander (non-target) sequence or gene.

In certain embodiments, a dsRNAi oligonucleotide which has been evaluated and tested for its ability to provide a particular biological effect (e.g., reduction of level, expression and/or activity of a target gene or a gene product thereof) can be used to treat, ameliorate and/or prevent a condition, disorder or disease.

### 9. Biologically Active Oligonucleotides

In certain embodiments, the present disclosure encompasses ds oligonucleotides which capable of acting as dsRNAi agents.

In certain embodiments, provided compositions include one or more oligonucleotides fully or partially complementary to a strand of: structural genes, genes control and/or termination regions, and/or self-replicating systems such as viral or plasmid DNA. In certain embodiments, provided compositions include one or more oligonucleotides that are or act as RNAi agents or other RNA interference reagents (RNAi agents or iRNA agents), shRNA, antisense oligonucleotides, self-cleaving RNAs, ribozymes, fragment thereof and/or variants thereof (such as Peptidyl transferase 23S rRNA, RNase P, Group I and Group II introns, GIR1 branching ribozymes, Leadzyme, Hairpin ribozymes, Hammerhead ribozymes, HDV ribozymes, Mammalian CPEB3 ribozyme, VS ribozymes, glmS ribozymes, CoTC ribozyme, etc.), microRNAs, microRNA mimics, supermirs, aptamers, antimirs, antagomirs, Ul adaptors, triplex-forming oligonucleotides, RNA activators, long non-coding RNAs, short non-coding RNAs (e.g., piRNAs), immunomodulatory oligonucleotides (such as immunostimulatory oligonucleotides, immunoinhibitory oligonucleotides), GNA, LNA, ENA, PNA, TNA, morpholinos, G-quadruplex (RNA and DNA), antiviral oligonucleotides, and decoy oligonucleotides.

In certain embodiments, provided compositions include one or more hybrid (e.g., chimeric) oligonucleotides. In the context of the present disclosure, the term “hybrid” broadly refers to mixed structural elements of oligonucleotides. Hybrid oligonucleotides may refer to, for example, (1) an oligonucleotide molecule having mixed classes of nucleotides, e.g., part DNA and part RNA within the single molecule (e.g., DNA-RNA); (2) complementary pairs of nucleic acids of different classes, such that DNA:RNA base pairing occurs either intramolecularly or intermolecularly; or both; (3) an oligonucleotide with two or more kinds of the backbone or internucleotide linkages.

In certain embodiments, provided compositions include one or more oligonucleotide that comprises more than one classes of nucleic acid residues within a single molecule. For example, in any of the embodiments described herein, an oligonucleotide may comprise a DNA portion and an RNA portion. In certain embodiments, an oligonucleotide may comprise an unmodified portion and modified portion.

Provided ds oligonucleotide compositions can include oligonucleotides containing any of a variety of modifications, for example as described herein. In certain embodiments, particular modifications are selected, for example, in light of intended use. In certain embodiments, it is desirable to modify one or both strands of a double-stranded oligonucleotide (or a double-stranded portion of a single-stranded oligonucleotide). In certain embodiments, the two strands (or portions) include different modifications. In certain embodiments, the two strands include the same modifications. One of skill in the art will appreciate that the degree and type of modifications enabled by methods of the present disclosure allow for numerous permutations of modifications to be made. Examples of such modifications are described herein and are not meant to be limiting.

The phrase “antisense strand” or “guide strand” as used herein, refers to an oligonucleotide that is substantially or 100% complementary to a target sequence of interest. The phrase “antisense strand” or “guide strand” includes the antisense region of both oligonucleotides that are formed from two separate strands, as well as unimolecular oligonucleotides that are capable of forming hairpin or dumbbell type structures. In reference to a double-stranded RNAi agent such as a siRNA, the antisense strand is the strand preferentially incorporated into RISC, and which targets RISC-mediated knockdown of an RNA target. In reference to a double-stranded RNAi agent, the terms “antisense strand” and “guide strand” are used interchangeably herein; and the terms “sense strand” or “passenger strand” are used interchangeably herein in reference to the strand which is not the antisense strand.

The phrase “sense strand” refers to an oligonucleotide that has the same nucleoside sequence, in whole or in part, as a target sequence such as a messenger RNA or a sequence of DNA.

By “target sequence” is meant any nucleic acid sequence whose expression or activity is to be modulated. The target nucleic acid can be DNA or RNA, such as endogenous DNA or RNA, viral DNA or viral RNA, or other RNA encoded by a gene, virus, bacteria, fungus, mammal, or plant. In certain embodiments, a target sequence is associated with a disease or disorder. In reference to RNA interference and RNase H-mediated knockdown, a target sequence is generally an RNA target sequence.

By “specifically hybridizable” and “complementary” is meant that a nucleic acid can form hydrogen bond(s) with another nucleic acid sequence by either traditional Watson-Crick or other non-traditional types. In reference to the nucleic molecules of the present disclosure, the binding free energy for a nucleic acid molecule with its complementary sequence is sufficient to allow the relevant function of the nucleic acid to proceed, e.g., RNAi activity. Determination of binding free energies for nucleic acid molecules is well known in the art (see, e.g., Turner et al, 1987, CSH Symp. Quant. Biol. LIT pp. 123-133; Frier et al., 1986, Proc. Nat. Acad. Sci. USA83:9373-9377; Turner et al., 1987, J. Am. Chem. Soc. 109:3783-3785)

A percent complementarity indicates the percentage of contiguous residues in a nucleic acid molecule that can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). “Perfectly complementary” or 100% complementarity means that all the contiguous residues of a nucleic acid sequence will hydrogen bond with the same number of contiguous residues in a second nucleic acid sequence. Less than perfect complementarity refers to the situation in which some, but not all, nucleoside units of two strands can hydrogen bond with each other. “Substantial complementarity” refers to polynucleotide strands exhibiting 90% r greater complementarity, excluding regions of the polynucleotide strands, such as overhangs, that are selected so as to be noncomplementary. Specific binding requires a sufficient degree of complementarity to avoid non-specific binding of the oligomeric compound to non-target sequences under conditions in which specific binding is desired, e.g., under physiological conditions in the case of in vivo assays or therapeutic treatment, or in the case of in vitro assays, under conditions in which the assays are performed. In certain embodiments, non-target sequences differ from corresponding target sequences by at least 5 nucleotides.

When used as therapeutics, a provided ds oligonucleotide is administered as a pharmaceutical composition. In certain embodiments, the pharmaceutical composition comprises a therapeutically effective amount of a provided oligonucleotide comprising, or a pharmaceutically acceptable salt thereof, and at least one pharmaceutically acceptable inactive ingredient selected from pharmaceutically acceptable diluents, pharmaceutically acceptable excipients, and pharmaceutically acceptable carriers. In certain embodiments, the pharmaceutical composition is formulated for intravenous injection, oral administration, buccal administration, inhalation, nasal administration, topical administration, ophthalmic administration or otic administration. In further embodiments, the pharmaceutical composition is a tablet, a pill, a capsule, a liquid, an inhalant, a nasal spray solution, a suppository, a suspension, a gel, a colloid, a dispersion, a suspension, a solution, an emulsion, an ointment, a lotion, an eye drop or an ear drop.

### 10. Administration of Oligonucleotides and Compositions

Many delivery methods, regimen, etc. can be utilized in accordance with the present disclosure for administering provided ds oligonucleotides and compositions thereof (typically pharmaceutical compositions for therapeutic purposes), including various technologies known in the art.

In certain embodiments, a ds oligonucleotide composition, e.g., a dsRNAi oligonucleotide composition, is administered at a dose and/or frequency lower than that of an otherwise comparable reference ds oligonucleotide composition and has comparable or improved effects. In certain embodiments, a chirally controlled ds oligonucleotide composition is administered at a dose and/or frequency lower than that of a comparable, otherwise identical stereorandom reference ds oligonucleotide composition and with comparable or improved effects, e.g., in improving the knockdown of the target transcript.

In certain embodiments, the present disclosure recognizes that properties and activities, e.g., knockdown activity, stability, toxicity, etc. of ds oligonucleotides and compositions thereof can be modulated and optimized by chemical modifications and/or stereochemistry. In certain embodiments, the present disclosure provides methods for optimizing ds oligonucleotide properties and/or activities through chemical modifications and/or stereochemistry. In certain embodiments, the present disclosure provides ds oligonucleotides and compositions thereof with improved properties and/or activities. Without wishing to be bound by any theory, due to, e.g., their better activity, stability, delivery, distribution, toxicity, pharmacokinetic, pharmacodynamics and/or efficacy profiles, Applicant notes that provided ds oligonucleotides and compositions thereof in certain embodiments can be administered at lower dosage and/or reduced frequency to achieve comparable or better efficacy, and in certain embodiments can be administered at higher dosage and/or increased frequency to provide enhanced effects. In certain embodiments, the present disclosure provides chirally controlled ds oligonucleotides and compositions thereof, wherein the chirally controlled ds oligonucleotides and compositions thereof do not exhibit increased off-target effects relative non-chirally controlled ds oligonucleotides. Moreover, in certain embodiments, the present disclosure provides chirally controlled ds oligonucleotides and compositions thereof, wherein the chirally controlled ds oligonucleotides and compositions thereof exhibit increased Ago2 loading of guide strand relative non-chirally controlled ds oligonucleotides.

In certain embodiments, the present disclosure provides, in a method of administering a ds oligonucleotide composition comprising a plurality of ds oligonucleotides sharing a common base sequence, the improvement comprising administering a ds oligonucleotide comprising a plurality of ds oligonucleotides that is characterized by improved delivery relative to a reference ds oligonucleotide composition of the same common base sequence.

In certain embodiments, provided ds oligonucleotides, compositions and methods provide improved delivery. In certain embodiments, provided ds oligonucleotides, compositions and methods provide improved cytoplasmatic delivery. In certain embodiments, improved delivery is to a population of cells. In certain embodiments, improved delivery is to a tissue. In certain embodiments, improved delivery is to an organ. In certain embodiments, improved delivery is to an organism, e.g., a patient or subject. Example structural elements (e.g., chemical modifications, stereochemistry, combinations thereof, etc.), oligonucleotides, compositions and methods that provide improved delivery are extensively described in the present disclosure.

In certain embodiments, provided ds oligonucleotides, compositions and methods can be delivered to various regions of the central nervous system. For example, but not by way of limitation, in certain embodiments, provided ds oligonucleotides, compositions and methods can be delivered to various regions of the brain. In certain embodiments, provided ds oligonucleotides, compositions and methods can be delivered to the cerebellum. In certain embodiments, provided ds oligonucleotides, compositions and methods can be delivered to the hippocampus. In certain embodiments, provided ds oligonucleotides, compositions and methods can be delivered to the cortex. In certain embodiments, provided ds oligonucleotides, compositions and methods can be delivered to the striatum. In certain embodiments, provided ds oligonucleotides, compositions and methods can be delivered to the spinal cord. In certain embodiments, provided ds oligonucleotides, compositions and methods can be delivered to the brain stem.

In certain embodiments, provided ds oligonucleotides, compositions and methods can be delivered to various regions of the central nervous system without conjugation to a targeting ligand (e.g., peptide, sugar, etc.), without a delivery system (e.g., lipid nanoparticles, etc.), or combinations thereof.

Various dosing regimens can be utilized to administer ds oligonucleotides and compositions of the present disclosure. In certain embodiments, multiple unit doses are administered, separated by periods of time. In certain embodiments, the present disclosure provides chirally controlled ds oligonucleotides and compositions thereof, wherein the chirally controlled ds oligonucleotides and compositions thereof do not exhibit diminished attributes relative non-chirally controlled ds oligonucleotides upon repeated dosing. For example, but not by way of limitation, such attributes can comprise one or more markers of liver function. Exemplary, markers of liver function include, but are not limited to ALT, alanine transaminase; AST, aspartate transaminase; ALP, alkaline phosphatase; ALB, albumin; TP, total protein. In certain embodiments, a given composition has a recommended dosing regimen, which may involve one or more doses. In certain embodiments, a dosing regimen comprises a plurality of doses each of which are separated from one another by a time period of the same length; in certain embodiments, a dosing regimen comprises a plurality of doses and at least two different time periods separating individual doses. In certain embodiments, all doses within a dosing regimen are of the same unit dose amount. In certain embodiments, different doses within a dosing regimen are of different amounts. In certain embodiments, a dosing regimen comprises a first dose in a first dose amount, followed by one or more additional doses in a second dose amount different from the first dose amount. In certain embodiments, a dosing regimen comprises a first dose in a first dose amount, followed by one or more additional doses in a second (or subsequent) dose amount that is the same as or different from the first dose (or another prior dose) amount. In certain embodiments, a chirally controlled ds oligonucleotide composition is administered according to a dosing regimen that differs from that utilized for a non-chirally controlled (e.g., stereorandom) ds oligonucleotide composition of the same sequence, and/or of a different chirally controlled ds oligonucleotide composition of the same sequence. In certain embodiments, a chirally controlled ds oligonucleotide composition is administered according to a dosing regimen that is reduced as compared with that of a chirally uncontrolled (e.g., stereorandom) ds oligonucleotide composition of the same sequence in that it achieves a lower level of total exposure over a given unit of time, involves one or more lower unit doses, and/or includes a smaller number of doses over a given unit of time. In certain embodiments, a chirally uncontrolled ds oligonucleotide is administered according to a dosing regimen that extends for a longer period of time than does that of a chirally uncontrolled (e.g., stereorandom) ds oligonucleotide composition of the same sequence. Without wishing to be limited by theory, Applicant notes that in certain embodiments, the shorter dosing regimen, and/or longer time periods between doses, may be due to the improved stability, bioavailability, and/or efficacy of a chirally controlled ds oligonucleotide composition. In certain embodiments, with their improved delivery (and other properties), provided compositions can be administered in lower dosages and/or with lower frequency to achieve biological effects, for example, clinical efficacy.

In certain embodiments, provided ds oligonucleotides, compositions and methods provide durable effects. In certain embodiments, the compositions of the present disclosure can maintain efficacy for up to 14 days. In certain embodiments, the compositions of the present disclosure can maintain efficacy for up to 56 days. In certain embodiments, durable effects can be achieved in the absence of substantial neurotoxicity. For example, but not by way of limitatation, such durable effects can be achieved while eliciting little or no detectable or only transiently detectable elevation of neuroinflammation markers and little or no detectable decrease in markers of Purkinje cell loss. In certain embodiments, there is no significant expression changes observed in Purkinjie cell markers associated with neurotoxicity. In certain embodiments, such durable effects can be achieved in the absence of conjugation of the ds oligonucleotide to a targeting ligand (e.g., peptide, sugar, etc.), without a delivery system (e.g., lipid nanoparticles, etc.), or combinations thereof.

### 11. Pharmaceutical Compositions

When used as therapeutics, a provided ds oligonucleotide, e.g., a dsRNAi oligonucleotide, or ds oligonucleotide composition thereof is typically administered as a pharmaceutical composition. In certain embodiments, the present disclosure provides pharmaceutical compositions comprising a provided compound, e.g., a ds oligonucleotide, or a pharmaceutically acceptable salt thereof, and a pharmaceutical carrier. In certain embodiments, for therapeutic and clinical purposes, ds oligonucleotides of the present disclosure are provided as pharmaceutical compositions. As appreciated by those skilled in the art, ds oligonucleotides of the present disclosure can be provided in their acid, base or salt forms. In certain embodiments, ds oligonucleotides can be in acid forms, e.g., for natural phosphate linkages, in the form of —OP(O)(OH)O—; for phosphorothioate internucleotidic linkages, in the form of —OP(O)(SH)O—; etc. In certain embodiments, dsRNAi oligonucleotides can be in salt forms, e.g., for natural phosphate linkages, in the form of —OP(O)(ONa)O— in sodium salts; for phosphorothioate internucleotidic linkages, in the form of —OP(O)(SNa)O— in sodium salts; etc. Unless otherwise noted, ds oligonucleotides of the present disclosure can exist in acid, base and/or salt forms.

In certain embodiments, a pharmaceutical composition is a liquid composition. In certain embodiments, a pharmaceutical composition is provided by dissolving a solid ds oligonucleotide composition, or diluting a concentrated ds oligonucleotide composition, using a suitable solvent, e.g., water or a pharmaceutically acceptable buffer. In certain embodiments, liquid compositions comprise anionic forms of provided ds oligonucleotides and one or more cations. In certain embodiments, liquid compositions have pH values in the weak acidic, about neutral, or basic range. In certain embodiments, pH of a liquid composition is about a physiological pH, e.g., about 7.4.

In certain embodiments, a provided ds oligonucleotide is formulated for administration to and/or contact with a body cell and/or tissue expressing its target. For example, in certain embodiments, a provided dsRNAi oligonucleotide is formulated for administration to a body cell and/or tissue. In certain embodiments such a body cell and/or tissue is selected from the group consisting of immune cells, blood cells, cardiac cells, lung cells, muscle cells, optic cells, liver cells, kidney cells, brain cells, cells of the central nervous system, and cells of the peripheral nervous system. In certain embodiments, such a body cell and/or tissue are a neuron or a cell and/or tissue of the liver. In certain embodiments, broad distribution of ds oligonucleotides and compositions may be achieved with intraparenchymal administration, intrathecal administration, or intracerebroventricular administration. In certain embodiments, the pharmaceutical composition is formulated for intravenous injection, oral administration, buccal administration, inhalation, nasal administration, topical administration, ophthalmic administration or optic administration. In certain embodiments, the pharmaceutical composition is a tablet, a pill, a capsule, a liquid, an inhalant, a nasal spray solution, a suppository, a suspension, a gel, a colloid, a dispersion, a suspension, a solution, an emulsion, an ointment, a lotion, an eye drop or an ear drop.

In certain embodiments, the present disclosure provides a pharmaceutical composition comprising chirally controlled ds oligonucleotide or composition thereof, in admixture with a pharmaceutically acceptable inactive ingredient (e.g., a pharmaceutically acceptable excipient, a pharmaceutically acceptable carrier, etc.). One of skill in the art will recognize that the pharmaceutical compositions include pharmaceutically acceptable salts of provided ds oligonucleotide or compositions. In certain embodiments, a pharmaceutical composition is a chirally controlled ds oligonucleotide composition. In certain embodiments, a pharmaceutical composition is a stereopure ds oligonucleotide composition.

In certain embodiments, the present disclosure provides salts of ds oligonucleotides and pharmaceutical compositions thereof. In certain embodiments, a salt is a pharmaceutically acceptable salt. In certain embodiments, a pharmaceutical composition comprises a ds oligonucleotide, optionally in its salt form, and a sodium salt. In certain embodiments, a pharmaceutical composition comprises a ds oligonucleotide, optionally in its salt form, and sodium chloride. In certain embodiments, each hydrogen ion of a ds oligonucleotide that may be donated to a base (e.g., under conditions of an aqueous solution, a pharmaceutical composition, etc.) is replaced by a non-H⁺ cation. For example, in certain embodiments, a pharmaceutically acceptable salt of a ds oligonucleotide is an all-metal ion salt, wherein each hydrogen ion (for example, of —OH, —SH, etc.) of each internucleotidic linkage (e.g., a natural phosphate linkage, a phosphoryl guanidine internucleotidic linkage, a phosphorothioate internucleotidic linkage, etc.) is replaced by a metal ion. Various suitable metal salts for pharmaceutical compositions are widely known in the art and can be utilized in accordance with the present disclosure. In certain embodiments, a pharmaceutically acceptable salt is a sodium salt. In certain embodiments, a pharmaceutically acceptable salt is magnesium salt. In certain embodiments, a pharmaceutically acceptable salt is a calcium salt. In certain embodiments, a pharmaceutically acceptable salt is a potassium salt. In certain embodiments, a pharmaceutically acceptable salt is an ammonium salt (cation N(R)₄⁺). In certain embodiments, a pharmaceutically acceptable salt comprises one and no more than one types of cation. In certain embodiments, a pharmaceutically acceptable salt comprises two or more types of cation. In certain embodiments, a cation is Li⁺, Na⁺, K⁺, Mg²⁺ or Ca²⁺. In certain embodiments, a pharmaceutically acceptable salt is an all-sodium salt. In certain embodiments, a pharmaceutically acceptable salt is an all-sodium salt, wherein each internucleotidic linkage which is a natural phosphate linkage (acid form —O—P(O)(OH)—O—), if any, exists as its sodium salt form (—O—P(O)(ONa)—O—), and each internucleotidic linkage which is a phosphorothioate or a phosphoryl guanidine internucleotidic linkage (acid form —O—P(O)(SH)—O—), if any, exists as its sodium salt form (—O—P(O)(SNa)—O—).

Various technologies for delivering nucleic acids and/or oligonucleotides are known in the art can be utilized in accordance with the present disclosure. For example, a variety of supramolecular nanocarriers can be used to deliver nucleic acids. Example nanocarriers include, but are not limited to liposomes, cationic polymer complexes and various polymeric compounds. Complexation of nucleic acids with various polycations is another approach for intracellular delivery; this includes use of PEGylated polycations, polyethyleneamine (PEI) complexes, cationic block co-polymers, and dendrimers. Several cationic nanocarriers, including PEI and polyamidoamine dendrimers help to release contents from endosomes. Other approaches include use of polymeric nanoparticles, microspheres, liposomes, dendrimers, biodegradable polymers, conjugates, prodrugs, inorganic colloids such as sulfur or iron, antibodies, implants, biodegradable implants, biodegradable microspheres, osmotically controlled implants, lipid nanoparticles, emulsions, oily solutions, aqueous solutions, biodegradable polymers, poly(lactide-coglycolic acid), poly(lactic acid), liquid depot, polymer micelles, quantum dots and lipoplexes. In certain embodiments, a ds oligonucleotide is conjugated to another molecule.

In therapeutic and/or diagnostic applications, compounds, e.g., ds oligonucleotides, of the disclosure can be formulated for a variety of modes of administration, including systemic and topical or localized administration. Techniques and formulations generally may be found in Remington, The Science and Practice of Pharmacy (20th ed. 2000).

Pharmaceutically acceptable salts for basic moieties are generally well known to those of ordinary skill in the art, and may include, e.g., acetate, benzenesulfonate, besylate, benzoate, bicarbonate, bitartrate, bromide, calcium edetate, camsylate, carbonate, citrate, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isethionate, lactate, lactobionate, malate, maleate, mandelate, mesylate, mucate, napsylate, nitrate, pamoate (embonate), pantothenate, phosphate/diphosphate, polygalacturonate, salicylate, stearate, subacetate, succinate, sulfate, tannate, tartrate, or teoclate. Other pharmaceutically acceptable salts may be found in, for example, Remington, The Science and Practice of Pharmacy (20th ed. 2000). Preferred pharmaceutically acceptable salts include, for example, acetate, benzoate, bromide, carbonate, citrate, gluconate, hydrobromide, hydrochloride, maleate, mesylate, napsylate, pamoate (embonate), phosphate, salicylate, succinate, sulfate, or tartrate.

In certain embodiments, dsRNAi oligonucleotides are formulated in pharmaceutical compositions described in WO 2005/060697, WO 2011/076807 or WO 2014/136086.

Depending on the specific conditions, disorders or diseases being treated, provided agents, e.g., ds oligonucleotides, may be formulated into liquid or solid dosage forms and administered systemically or locally. Provided ds oligonucleotides may be delivered, for example, in a timed- or sustained-low release form as is known to those skilled in the art. Techniques for formulation and administration may be found in Remington, The Science and Practice of Pharmacy (20th ed. 2000). Suitable routes may include oral, buccal, by inhalation spray, sublingual, rectal, transdermal, vaginal, transmucosal, nasal or intestinal administration; parenteral delivery, including intramuscular, subcutaneous, intramedullary injections, as well as intrathecal, direct intraventricular, intravenous, intra-articullar, intra-stemal, intra-synovial, intra-hepatic, intralesional, intracranial, intraperitoneal, intranasal, or intraocular injections or another mode of delivery.

For injection, provided agents, e.g., oligonucleotides may be formulated and diluted in aqueous solutions, such as in physiologically compatible buffers such as Hank's solution, Ringer's solution, or physiological saline buffer. For such transmucosal administration, penetrants appropriate to the barrier to be permeated are used in the formulations. Such penetrants are generally known in the art and can be utilized in accordance with the present disclosure.

Use of pharmaceutically acceptable carriers to formulate compounds, e.g., provided ds oligonucleotides, for the practice of the disclosure into dosages suitable for various mods of administration is well known in the art. With proper choice of carrier and suitable manufacturing practice, compositions of the present disclosure, e.g., those formulated as solutions, may be administered via various routes, e.g., parenterally, such as by intravenous injection.

In certain embodiments, a composition comprising a dsRNAi oligonucleotide further comprises any or all of calcium chloride dihydrate, magnesium chloride hexahydrate, potassium chloride, sodium chloride, sodium phosphate dibasic anhydrous, sodium phosphate, monobasic dihydrate, and/or water for Injection. In certain embodiments, a composition further comprises any or all of calcium chloride dihydrate (0.21 mg) USP, magnesium chloride hexahydrate (0.16 mg) USP, potassium chloride (0.22 mg) USP, sodium chloride (8.77 mg) USP, sodium phosphate dibasic anhydrous (0.10 mg) USP, sodium phosphate monobasic dihydrate (0.05 mg) USP, and Water for Injection USP.

In certain embodiments, a composition comprising a ds oligonucleotide further comprises any or all of: cholesterol, (6Z,9Z,28Z,31Z)-heptatriaconta-6,9,28,31-tetraen-19-yl-4-(dimethylamino) butanoate(DLin-MC3-DMA), 1,2-distearoyl-sn-glycero-3-phosphocholine (DSPC), alpha-(3′-{[1,2-di(myristyloxy)propanoxy]carbonylamino}propyl)-omega-methoxy, polyoxyethylene(PEG2000-C-DMG), potassium phosphate monobasic anhydrous NF, sodium chloride, sodium phosphate dibasic heptahydrate, and Water for Injection. In certain embodiments, the pH of a composition comprising a RNAi oligonucleotide is -7.0. In certain embodiments, a composition comprising an oligonucleotide further comprises any or all of: 6.2 mg cholesterol USP, 13.0 mg (6Z,9Z,28Z,31Z)-heptatriaconta-6,9,28,31-tetraen-19-yl-4-(dimethylamino) butanoate(DLin-MC3-DMA), 3.3 mg 1,2-distearoyl-sn-glycero-3-phosphocholine (DSPC), 1.6 mg α-(3′-{[1,2-di(myristyloxy)propanoxy]carbonylamino}propyl)-ω-methoxy, polyoxyethylene(PEG2000—C-DMG), 0.2 mg potassium phosphate monobasic anhydrous NF, 8.8 mg sodium chloride USP, 2.3 mg sodium phosphate dibasic heptahydrate USP, and Water for Injection USP, in an approximately 1 mL total volume.

Provided compounds, e.g., ds oligonucleotides, can be formulated readily using pharmaceutically acceptable carriers well known in the art into dosages suitable for oral administration. In certain embodiments, such carriers enable provided oligonucleotides to be formulated as tablets, pills, capsules, liquids, gels, syrups, slurries, suspensions and the like, for, e.g., oral ingestion by a subject (e.g., patient) to be treated.

For nasal or inhalation delivery, provided compounds, e.g., ds oligonucleotides, may be formulated by methods known to those of skill in the art, and may include, e.g., examples of solubilizing, diluting, or dispersing substances such as saline, preservatives, such as benzyl alcohol, absorption promoters, and fluorocarbons.

In certain embodiments, methods of specifically localizing provided compounds, e.g., ds oligonucleotides, such as by bolus injection, may decrease median effective concentration (EC50) by a factor of 20, 25, 30, 35, 40, 45 or 50. In certain embodiments, a targeted tissue is brain tissue. In certain embodiments, a targeted tissue is striatal tissue. In certain embodiments, decreasing EC50 is desirable because it reduces the dose required to achieve a pharmacological result in a patient in need thereof.

In certain embodiments, a provided ds oligonucleotide is delivered by injection or infusion once every month, every two months, every 90 days, every 3 months, every 6 months, twice a year or once a year.

Pharmaceutical compositions suitable for use in the present disclosure include compositions wherein the active ingredients, e.g., ds oligonucleotides, are contained in effective amounts to achieve their intended purposes. Determination of the effective amounts is well within the capability of those skilled in the art, especially in light of the detailed disclosure provided herein.

In addition to active ingredients, pharmaceutical compositions may contain suitable pharmaceutically acceptable carriers comprising excipients and auxiliaries which facilitate processing of an active compound into preparations which can be used pharmaceutically. Preparations formulated for oral administration may be in the form of tablets, dragees, capsules, or solutions.

In certain embodiments, pharmaceutical compositions for oral use can be obtained by combining an active compound with solid excipients, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations, for example, maize starch, wheat starch, rice starch, potato starch, gelatin, gum tragacanth, methyl cellulose, hydroxypropylmethyl-cellulose, sodium carboxymethyl-cellulose (CMC), and/or polyvinylpyrrolidone (PVP: povidone). If desired, disintegrating agents may be added, such as the cross-linked polyvinylpyrrolidone, agar, or alginic acid or a salt thereof such as sodium alginate.

In certain embodiments, dragee cores are provided with suitable coatings. For this purpose, concentrated sugar solutions may be used, which may optionally contain gum arabic, talc, polyvinylpyrrolidone, carbopol gel, polyethylene glycol (PEG), and/or titanium dioxide, lacquer solutions, and suitable organic solvents or solvent mixtures. Dye-stuffs or pigments may be added to the tablets or dragee coatings for identification or to characterize different combinations of active compound doses.

Pharmaceutical preparations that can be used orally include push-fit capsules made of gelatin, as well as soft, sealed capsules made of gelatin, and a plasticizer, such as glycerol or sorbitol. Push-fit capsules can contain active ingredients, e.g., ds oligonucleotides, in admixture with fillers such as lactose, binders such as starches, and/or lubricants such as talc or magnesium stearate and, optionally, stabilizers. In soft capsules, active compounds, e.g., ds oligonucleotides, may be dissolved or suspended in suitable liquids, such as fatty oils, liquid paraffin, or liquid polyethylene glycols (PEGs). In addition, stabilizers may be added.

In certain embodiments, a provided composition comprises a lipid. In certain embodiments, a lipid is conjugated to an active compound, e.g., an oligonucleotide. In certain embodiments, a lipid is not conjugated to an active compound. In certain embodiments, a lipid comprises a C₁₀-C₄₀ linear, saturated or partially unsaturated, aliphatic chain. In certain embodiments, a lipid comprises a C₁₀-C₄₀ linear, saturated or partially unsaturated, aliphatic chain, optionally substituted with one or more C₁₋₄ aliphatic group. In certain embodiments, the lipid is selected from the group consisting of lauric acid, myristic acid, palmitic acid, stearic acid, oleic acid, linoleic acid, alpha-linolenic acid, gamma-linolenic acid, docosahexaenoic acid (cis-DHA), turbinaric acid and dilinoleyl alcohol. In certain embodiments, an active compound is a provided oligonucleotide. In certain embodiments, a composition comprises a lipid and an active compound, and further comprises another component which is another lipid or a targeting compound or moiety. In certain embodiments, a lipid is an amino lipid; an amphipathic lipid; an anionic lipid; an apolipoprotein; a cationic lipid; a low molecular weight cationic lipid; a cationic lipid such as CLinDMA and DLinDMA; an ionizable cationic lipid; a cloaking component; a helper lipid; a lipopeptide; a neutral lipid; a neutral zwitterionic lipid; a hydrophobic small molecule; a hydrophobic vitamin; a PEG-lipid; an uncharged lipid modified with one or more hydrophilic polymers; phospholipid; a phospholipid such as 1,2-dioleoyl-sn-glycero-3-phosphoethanolamine; a stealth lipid; a sterol; a cholesterol; a targeting lipid; or another lipid described herein or reported in the art suitable for pharmaceutical uses. In certain embodiments, a composition comprises a lipid and a portion of another lipid capable of mediating at least one function of another lipid. In certain embodiments, the lipid comprises a C₁₂ linear, saturated, aliphatic chain. In certain embodiments, the lipid comprises a C₁₆ linear, saturated, aliphatic chain. In certain embodiments, the lipid is incorporated into a phosphoryl guanidine chiral center. In certain embodiments of the compositions and methods described herein, the composition does not comprise a lipid. In certain of such compositions that do not comprise a lipid, the activity of the composition that does not comprise a lipid is preserved relative to the same composition comprising a lipid.

In certain embodiments, a targeting compound or moiety is capable of targeting a compound (e.g., a ds oligonucleotide) to a particular cell or tissue or subset of cells or tissues. In certain embodiments, a targeting moiety is designed to take advantage of cell- or tissue-specific expression of particular targets, receptors, proteins, or another subcellular component. In certain embodiments, a targeting moiety is a ligand (e.g., a small molecule, antibody, peptide, protein, carbohydrate, aptamer, etc.) that targets a composition to a cell or tissue, and/or binds to a target, receptor, protein, or another subcellular component.

Certain example lipids for delivery of an active compound, e.g., a ds oligonucleotide, allow (e.g., do not prevent or interfere with) the function of an active compound. In certain embodiments, a lipid is lauric acid, myristic acid, palmitic acid, stearic acid, oleic acid, linoleic acid, alpha-linolenic acid, gamma-linolenic acid, docosahexaenoic acid (cis-DHA), turbinaric acid or dilinoleyl alcohol.

As described in the present disclosure, lipid conjugation, such as conjugation with fatty acids, may improve one or more properties of ds oligonucleotides.

In certain embodiments, a composition for delivery of an active compound, e.g., a ds oligonucleotide, is capable of targeting an active compound to particular cells or tissues as desired. In certain embodiments, a composition for delivery of an active compound is capable of targeting an active compound to a muscle cell or tissue. In certain embodiments, the present disclosure provides compositions and methods related to delivery of active compounds, wherein the compositions comprise an active compound and a lipid. In various embodiments to a hepatic cell or tissue, a lipid is selected from lauric acid, myristic acid, palmitic acid, stearic acid, oleic acid, linoleic acid, alpha-linolenic acid, gamma-linolenic acid, docosahexaenoic acid (cis-DHA), turbinaric acid and dilinoleyl alcohol.

In certain embodiments, a dsRNAi oligonucleotide is delivered to the central nervous or hepetic system, or a cell or tissue or portion thereof, via a delivery method or composition designed for delivery of nucleic acids to the central nervous or hepetic system, or a cell or tissue or portion thereof.

In certain embodiments, a dsRNAi oligonucleotide is delivered via a composition comprising any one or more of, or a method of delivery involving the use of any one or more of: transferrin receptor-targeted nanoparticle; cationic liposome-based delivery strategy; cationic liposome; polymeric nanoparticle; viral carrier; retrovirus; adeno-associated virus; stable nucleic acid lipid particle; polymer; cell-penetrating peptide; lipid; dendrimer; neutral lipid; cholesterol; lipid-like molecule; fusogenic lipid; hydrophilic molecule; polyethylene glycol (PEG) or a derivative thereof; shielding lipid; PEGylated lipid; PEG-C-DMSO; PEG-C-DMSA; DSPC; ionizable lipid; a guanidinium-based cholesterol derivative; ion-coated nanoparticle; metal-ion coated nanoparticle; manganese ion-coated nanoparticle; angubindin-1; nanogel; incorporation of the dsRNAi into a branched nucleic acid structure; and/or incorporation of the dsRNAi into a branched nucleic acid structure comprising 2, 3, 4 or more oligonucleotides.

In certain embodiments, a composition comprising a ds oligonucleotide is lyophilized. In certain embodiments, a composition comprising a ds oligonucleotide is lyophilized, and the lyophilized ds oligonucleotide is in a vial. In certain embodiments, the vial is back filled with nitrogen. In certain embodiments, the lyophilized ds oligonucleotide composition is reconstituted prior to administration. In certain embodiments, the lyophilized ds oligonucleotide composition is reconstituted with a sodium chloride solution prior to administration. In certain embodiments, the lyophilized ds oligonucleotide composition is reconstituted with a 0.9% sodium chloride solution prior to administration. In certain embodiments, reconstitution occurs at the clinical site for administration. In certain embodiments, in a lyophilized composition, a ds oligonucleotide composition is chirally controlled or comprises at least one chirally controlled internucleotidic linkage and/or the ds oligonucleotide targets.

### II. EXEMPLIFICATION

Various technologies can be utilized to assess properties and/or activities of provided oligonucleotides and compositions thereof. Some such technologies are described in this Example. Those skilled in the art appreciate that many other technologies can be readily utilized. As demonstrated herein, provided oligonucleotides and compositions, among other things, can be highly active, e.g., in reducing levels of their target nucleic acids. Certain examples of provided technologies (compounds (oligonucleotides, reagents, etc.), compositions, methods (methods of preparation, use, assessment, etc.), etc.) were presented herein.

### Example 1. Oligonucleotide Synthesis

Various technologies for preparing oligonucleotides and oligonucleotide compositions (both stereorandom and chirally controlled) are known and can be utilized in accordance with the present disclosure, including, for example, those in U.S. Pat. Nos. 9,394,333, 9,744,183, 9,605,019, 9,598,458, 9,982,257, U.S. Ser. No. 10/160,969, U.S. Ser. No. 10/479,995, US 2020/0056173, US 2018/0216107, US 2019/0127733, U.S. Ser. No. 10/450,568, US 2019/0077817, US 2019/0249173, US 2019/0375774, WO 2018/223056, WO 2018/223073, WO 2018/223081, WO 2018/237194, WO 2019/032607, WO 2019/055951, WO 2019/075357, WO 2019/200185, WO 2019/217784, WO 2019/032612, WO 2020/191252, and/or WO 2021/071858, the methods and reagents of each of which are incorporated herein by reference. Stereorandom and chirally controlled guide strand sequences were prepared utilizing the synthetic procedures as exemplified in above mentioned disclosures. Respective passenger strands were designed to have covalently linked GalNAc moiety as delivery vehicle at either end of sequences. Oligonucleotides with 5′-GalNAc modifications were synthesized by coupling C₆-amino modifier linker at the 5′-end of sequence. Oligonucleotides with 3′-GalNAc moiety as delivery vehicle were synthesized by utilizing 3′-C6 amino modified support. The single strand was cleaved from CPG by using deprotection condition as exemplified in earlier disclosures. The resulting amino group containing crude oligonucleotide was purified by ion exchange chromatography on AKTA pure system using a sodium chloride gradient. Desired product was desalted and further used for conjugation with GalNAc acid. After conjugation reaction was found to be complete the material was further purified by ion exchange chromatography and desalted to achieve desired material. For introduction of PN linkages in guide and passenger strands, specific PN coupling cycles were introduced at desired positions in oligonucleotide sequence utilizing the conditions as exemplified in WO2019/200185.

In certain embodiments, oligonucleotides were prepared using suitable chiral auxiliaries, e.g., DPSE and PSM chiral auxiliaries. Various oligonucleotides, e.g., those in Table 1, and compositions thereof, were prepared in accordance with the present disclosure.

Various technologies can be utilized to assess properties and/or activities of provided oligonucleotides and compositions thereof. Some such technologies are described in this Example. Those skilled in the art appreciate that many other technologies can be readily utilized. As demonstrated herein, provided oligonucleotides and compositions, among other things, can be highly active, e.g., in reducing levels of their target nucleic acids.

### ABBREVIATION

1× reagent: TEA-3HF:TEA:H₂O: DMSO=5.0:1.8:15.5:77.7 (v/v/v/v)
ADIH: 2-azido-1,3-dimethylimidazolium hexafluorophosphate
CMIMT: N-cyanomethylimidazolium triflate
CPG: controlled pore glass
DCM: dichloromethane, CH₂Cl₂
DIPEA: diisopropylethylamine
DMSO: dimethylsulfoxide
DMTr: 4,4′-dimethoxytrityl
GalNAc: N-acetylgalactosamine
HF: hydrogen fluoride
HATU: 1-[bis(dimethylamino)methylene]-1H-1,2,3-triazolo[4,5-b]pyridinium 3-oxide hexafluorophosphate
IBN: isobutyronitrile
MeCN: acetonitrile
MeIm: N-methylimidazole
TCA: trichloroacetic acid
TEA: triethylamine
XH: xanthane hydride

### General Procedure for the Synthesis of Chiral-Oligos (25 μMol Scale)

The automated solid-phase synthesis of chiral-oligos was performed according to the cycles shown in Table 16 (regular amidite cycle, for PO linkages), Table 17 (regular amidite cycle, for stereo-random PS linkages), Table 18 (DPSE amidite cycle, for chiral PS linkages), and Table 19 (PSM amidite cycle, for chiral PN linkages).

TABLE 2
Regular Amidite Synthetic Cycle for PO linkages

| step | operation | reagents and solvent | volume | | waiting time | |
|---|---|---|---|---|---|---|
| 1 | detritylation | 3% TCA/DCM | 10 | mL | 65 | s |
| 2 | coupling | 0.2M monomer/20% IBN-MeCN | 0.5 | mL | 8 | min |
| | | 0.5M CMIMT/MeCN | 1.0 | mL | | |
| 3 | oxidation | 50 mM I₂/pyridine-H₂O (9:1, v/v) | 2.0 | mL | 1 | min |
| 4 | cap-2 | 20% Ac₂O, 30% 2,6-lutidine/MeCN | 1.0 | mL | 45 | s |

| | | |
|---|---|---|
| 20% MeIm/MeCN | 1.0 | mL |

TABLE 3
Regular Amidite Synthetic Cycle for stereo-random PS linkages

| step | operation | reagents and solvent | volume | | waiting time | |
|---|---|---|---|---|---|---|
| 1 | detritylation | 3% TCA/DCM | 10 | mL | 65 | s |
| 2 | coupling | 0.2M monomer/20% IBN-MeCN | 0.5 | mL | 8 | min |
| | | 0.5M CMIMT/MeCN | 1.0 | mL | | |
| 3 | sulfurization | 0.2M XH/pyridine | 2.0 | mL | 6 | min |
| 4 | cap-2 | 20% Ac₂O, 30% 2,6-lutidine/MeCN | 1.0 | mL | 45 | s |

| | | |
|---|---|---|
| 20% MeIm/MeCN | 1.0 | mL |

TABLE 4
DPSE Amidite Synthetic Cycle for chiral PS linkages

| step | operation | reagents and solvent | volume | | waiting time | |
|---|---|---|---|---|---|---|
| 1 | detritylation | 3% TCA/DCM | 10 | mL | 65 | s |
| 2 | coupling | 0.2M monomer/20% IBN-MeCN | 0.5 | mL | 8 | min |
| | | 0.5M CMIMT/MeCN | 1.0 | mL | | |
| 3 | cap-1 | 20% Ac₂O, 30% 2,6-lutidine/MeCN | 2.0 | mL | 2 | min |
| 4 | sulfurization | 0.2M XH/pyridine | 2.0 | mL | 6 | min |
| 5 | cap-2 | 20% Ac₂O, 30% 2,6-lutidine/MeCN | 1.0 | mL | 45 | s |

| | | |
|---|---|---|
| 20% MeIm/MeCN | 1.0 | mL |

TABLE 5
PSM Amidite Synthetic Cycle for chiral PN linkages

| step | operation | reagents and solvent | volume | | waiting time | |
|---|---|---|---|---|---|---|
| 1 | detritylation | 3% TCA/DCM | 10 | mL | 65 | s |
| 2 | coupling | 0.2M monomer/20% IBN-MeCN | 0.5 | mL | 8 | min |
| | | 0.5M CMIMT/MeCN | 1.0 | mL | | |
| 3 | cap-1 | 20% Ac₂O, 30% 2,6-lutidine/MeCN | 2.0 | mL | 2 | min |
| 4 | imidation | 0.5M ADIH reagent/MeCN | 2.0 | mL | 6 | min |
| 5 | cap-2 | 20% Ac₂O, 30% 2,6-lutidine/MeCN | 1.0 | mL | 45 | s |

| | | |
|---|---|---|
| 20% MeIm/MeCN | 1.0 | mL |

1. In some embodiments, preparations include one or more DPSE and/or PSM cycles

### General Procedure for the C&D Conditions (25 μmol Scale)

After completion of the synthesis, the CPG solid support was dried and transferred into 50 mL plastic tube. The CPG was treated with 1× reagent (2.5 mL; 100 μL/umol) for 3 h at 28° C., then added conc. NH₃ (5.0 mL; 200 μL/umol) for 24 h at 37° C. The reaction mixture was cooled to room temperature and the CPG was separated by membrane filtration, washed with 15 mL of H₂O. The crude material (filtrate) was analyzed by LTQ and RP-UPLC.

### General Procedure for the GalNAc Conjugation Conditions (1 μmol Scale)

Into a plastic tube, tri-GalNAc (2.0 eq.), HATU (1.9 eq.), and DIPEA (10 eq.) were dissolved in anhydrous MeCN (0.5 mL). The mixture was stirred for 10 min at room temperature, then the mixture was added into the amino-oligo (1 mol) in H₂O (1 mL) and stirred for 1 h at 37° C. The reaction was monitored by LC-MS and RP-UPLC. After the reaction was completed, the resultant GalNAc-conjugated oligo was treated with conc. NH₃ (2 mL) for 1 h at 37° C. The solution was concentrated under vacuum to remove MeCN and conc. NH₃. The residue was then dissolved in H₂O (10 mL) for reversed phase purification.

While various embodiments have been described and illustrated herein, those of ordinary skill in the art will readily envision a variety of other means and/or structures for performing the functions and/or obtaining the results and/or one or more of the advantages described in the present disclosure, and each of such variations and/or modifications is deemed to be included. More generally, those skilled in the art will readily appreciate that all parameters, dimensions, materials, and configurations described herein are meant to be example and that the actual parameters, dimensions, materials, and/or configurations may depend upon the specific application or applications for which the teachings of the present disclosure is/are used. Those skilled in the art will recognize, or be able to ascertain using no more than routine experimentation, many equivalents to the embodiments of the present disclosure. It is, therefore, to be understood that the foregoing embodiments are presented by way of example only and that, within the scope of the appended claims and equivalents thereto, claimed technologies may be practiced otherwise than as specifically described and claimed. In addition, any combination of two or more features, systems, articles, materials, kits, and/or methods, if such features, systems, articles, materials, kits, and/or methods are not mutually inconsistent, is included within the scope of the present disclosure.

### Example 2. Provided Oligonucleotides and Compositions are Active In Vivo

In vivo determination of mouse TTR siRNA activity: All animal procedures were performed under IACUC guidelines. To evaluate the potency and liver exposure of provided oligonucleotides and compositions, male 8-10 weeks of age C₅₇BL/6 mice were dose at 0.6, 2 or 6 mg/kg at desired oligonucleotide concentration on Day 1 by subcutaneous administration. Animals were euthanized on Day 8 by CO₂ asphyxiation followed by thoracotomy and terminal blood collection. After cardiac perfusion with PBS, liver samples were harvested and flash-frozen in dry ice. Liver total RNA was extracted using SV96 Total RNA Isolation kit (Promega), after tissue lysis with TRIzol and bromochloropropane. cDNA production from RNA samples were performed using High-Capacity cDNA Reverse Transcription kit (Thermo Fisher) following manufacturer's instructions and qPCR analysis performed in CFX System using iQ Multiplex Powermix (Bio-Rad). For mouse TTR mRNA, the following qPCR assay were utilized: IDT Taqman qPCR assay ID Mm.PT.58.11922308. Oligonucleotide accumulation in liver was determined by hybrid ELISA.

To evaluate the durability of provided oligonucleotides and compositions, male 8-10 weeks of age C57BL/6 mice were dose at 2 or 6 mg/kg at desired oligonucleotide concentration on Day 1 by subcutaneous administration. On Day 1 (pre-dose) and then weekly, whole blood was collected via submandibular bleeding into serum separator tubes, and processed serum samples were kept at −70° C. Mouse TTR protein concentration in the serum was assessed using the Mouse Prealbumin ELISA kit (Crystal Chem) and following manufacturer's instructions.

Ago2 immunoprecipitation assay: Tissues (1 mpk dosed) were lysed in lysis buffer 50 mM Tris-HCl at pH 7.5, 200 mM NaCl, 0.5% Triton X-100, 2 mM EDTA, 1 mg/mL heparin) with protease inhibitor (Sigma-Aldrich). Lysate concentration was measured with a protein BCA kit (Pierce BCA protein assay kit or Bradford protein assay kit). Anti-Ago2 antibody was purchased from Wako Chemicals. Control mouse IgG was from eBioscience. Dynabeads (Invitrogen) were used to precipitate antibodies. Ago2-associated siRNA and endogenous miR122 were measured by Stem-Loop RT followed by TaqMan PCR analysis using Taqman miRNA and siRNA assay kit (Thermo fisher) based on manufacturer's methods.

Table 6, shows % mouse TTR mRNA remaining relative to PBS control. N=5. N.D.: Not determined.

TABLE 6

| | WV-20167/WV-36860 | | |
|---|---|---|---|
| PBS | 0.6 mg/kg | 2 mg/kg | 6 mg/kg |

| | % remaining | | % remaining | | % remaining | | % remaining |
|---|---|---|---|---|---|---|---|
| animal | of mTTR | animal | of mTTR | animal | of mTTR | animal | of mTTR |
| No. | mRNA | No. | mRNA | No. | mRNA | No. | mRNA |
| 1 | 126.89 | 6 | 81.73 | 11 | 38.85 | 16 | 35.76 |
| 2 | 86.61 | 7 | 70.20 | 12 | 42.98 | 17 | 17.77 |
| 3 | 95.66 | 8 | 75.48 | 13 | 40.83 | 18 | 20.10 |
| 4 | 93.00 | 9 | 65.90 | 14 | 48.03 | 19 | 11.25 |
| 5 | 97.84 | 10 | 77.26 | 15 | 42.39 | 20 | 10.30 |
| Mean | 100 | Mean | 74.11 | Mean | 42.62 | Mean | 19.03 |

| WV-20170/WV-36807 | | | WV-38708/WV-36807 |
|---|---|---|---|
| 0.6 mg/kg | 2 mg/kg | 6 mg/kg | 0.6 mg/kg |

| | % remaining | | % remaining | | % remaining | | % remaining |
|---|---|---|---|---|---|---|---|
| animal | of mTTR | animal | of mTTR | animal | of mTTR | animal | of mTTR |
| No. | mRNA | No. | mRNA | No. | mRNA | No. | mRNA |
| 21 | 59.04 | 26 | 27.62 | 31 | 8.38 | 36 | 74.11 |
| 22 | 64.79 | 27 | 24.76 | 32 | 9.90 | 37 | 51.80 |
| 23 | 57.41 | 28 | 39.43 | 33 | 4.61 | 38 | 75.30 |
| 24 | 63.78 | 29 | 33.50 | 34 | 6.79 | 39 | 58.13 |
| 25 | 61.99 | 30 | 28.70 | 35 | 14.13 | 40 | 75.99 |
| Mean | 61.40 | Mean | 30.80 | Mean | 8.76 | Mean | 67.07 |

WV-38708/WV-36807

| | |
|---|---|
| 2 mg/kg | 6 mg/kg |

| | % remaining | | % remaining |
|---|---|---|---|
| animal | of mTTR | animal | of mTTR |
| No. | mRNA | No. | mRNA |
| 41 | 14.89 | 46 | 3.01 |
| 42 | 12.19 | 47 | 5.27 |
| 43 | 19.18 | 48 | 3.76 |
| 44 | 23.48 | 49 | 2.98 |
| 45 | 17.54 | 50 | 3.94 |
| Mean | 17.46 | Mean | 3.79 |

Table 7, shows the accumulation of antisense strand in liver tissue. N=5. N.D.: Not determined.

TABLE 7

| | | WV-20167/WV-36860 | | | | | |
|---|---|---|---|---|---|---|---|
| | | | | 2 mg/kg | | | |
| | PBS | 0.6 mg/kg | | | % | 6 mg/kg | |
| | antisense | | antisense | | antisense | | antisense |
| | strand | | strand | | strand | | strand |
| animal | (μg/g of | animal | (μg/g of | animal | (μg/g of | animal | (μg/g of |
| No. | tissue) | No. | tissue) | No. | tissue) | No. | tissue) |
| 1 | 0.000 | 6 | 0.024 | 11 | 0.031 | 16 | 0.055 |
| 2 | 0.000 | 7 | 0.048 | 12 | 0.084 | 17 | 0.121 |
| 3 | 0.000 | 8 | 0.032 | 13 | 0.069 | 18 | 0.113 |
| 4 | 0.000 | 9 | 0.025 | 14 | 0.049 | 19 | 0.101 |
| 5 | 0.000 | 10 | 0.026 | 15 | 0.042 | 20 | 0.131 |
| Mean | 0.000 | Mean | 0.031 | Mean | 0.055 | Mean | 0.104 |

| WV-20170/WV-36807 | | | | | | WV-38708/WV-36807 | |
|---|---|---|---|---|---|---|---|
| 0.6 mg/kg | | 2 mg/kg | | 6 mg/kg | | 0.6 mg/kg | |
| | antisense | | antisense | | antisense | | antisense |
| | strand | | strand | | strand | | strand |
| animal | (μg/g of | animal | (μg/g of | animal | (μg/g of | animal | (μg/g of |
| No. | tissue) | No. | tissue) | No. | tissue) | No. | tissue) |
| 21 | 0.040 | 26 | 0.066 | 31 | 0.057 | 36 | 0.017 |
| 22 | 0.024 | 27 | 0.032 | 32 | 0.062 | 37 | 0.015 |
| 23 | 0.006 | 28 | 0.015 | 33 | 0.110 | 38 | 0.011 |
| 24 | 0.000 | 29 | 0.019 | 34 | 0.072 | 39 | 0.004 |
| 25 | 0.000 | 30 | 0.022 | 35 | 0.103 | 40 | 0.010 |
| Mean | 0.014 | Mean | 0.031 | Mean | 0.081 | Mean | 0.012 |

WV-38708/WV-36807

| 2 mg/kg | | 6 mg/kg | |
|---|---|---|---|
| | antisense | | antisense |
| | strand | | strand |
| animal | (μg/g of | animal | (μg/g of |
| No. | tissue) | No. | tissue) |
| 41 | 0.063 | 46 | 0.298 |
| 42 | 0.088 | 47 | 0.365 |
| 43 | 0.065 | 48 | 0.196 |
| 44 | 0.092 | 49 | 0.229 |
| 45 | 0.050 | 50 | 0.322 |
| Mean | 0.072 | Mean | 0.282 |

Table 8. shows Ago 2 loading of guide strand relative to miR-122. N=2.

| | Ct: | Ct: miR- | Ct | Ct: miR- | Relative |
|---|---|---|---|---|---|
| | mTTR/Ago2 | 122/Ago2 | mTTR/IgG | 122/IgG | mTTR/miR122 |
| PBS-1 | 39.07 | 16.86 | 38.67 | 27.78 | −0.02 |
| PBS-2 | 38.86 | 16.95 | 39.06 | 28.16 | 0.01 |
| WV-20167-1 | 36.57 | 16.94 | 38.63 | 28.58 | 0.34 |
| WV-20167-2 | 36.40 | 16.90 | 38.87 | 28.55 | 0.40 |
| WV-20170-1 | 34.56 | 16.63 | 38.67 | 27.49 | 1.35 |
| WV-20170-2 | 33.91 | 16.91 | 38.93 | 27.80 | 2.64 |
| WV-38708-1 | 31.10 | 16.48 | 36.70 | 28.02 | 13.94 |
| WV-38708-2 | 31.15 | 16.37 | 35.22 | 27.33 | 11.90 |

Table 8a. shows % mouse TTR protein remaining relative to PBS control. N=5. N.D.: Not determined.

| | PBS | | | | | |
|---|---|---|---|---|---|---|
| Day | animal1 | animal2 | animal3 | animal4 | animal5 | Mean |
| 1 | 91 | 114 | 104 | 91 | 100 | 100 |
| 8 | 103 | 120 | 98 | 91 | 87 | 100 |
| 15 | 91 | 100 | 112 | 103 | 93 | 100 |
| 22 | 94 | 107 | 91 | 102 | 106 | 100 |
| 29 | 93 | 101 | 87 | 108 | 111 | 100 |
| 36 | 87 | 102 | 95 | 112 | 104 | 100 |
| 43 | 98 | 95 | 95 | 101 | 112 | 100 |

| | WV-20167/WV-36860, 2 mg/kg | | | | | |
|---|---|---|---|---|---|---|
| Day | animal6 | animal7 | animal8 | animal9 | animal10 | Mean |
| 1 | 97 | 105 | 72 | 80 | 90 | 89 |
| 8 | 54 | 48 | 39 | 51 | 44 | 47 |
| 15 | 68 | 70 | 62 | 62 | 63 | 65 |
| 22 | 90 | 96 | 80 | 92 | 92 | 90 |
| 29 | 85 | 116 | 82 | 98 | 91 | 94 |
| 36 | 115 | 120 | 108 | 88 | 122 | 110 |
| 43 | 102 | 115 | 99 | 99 | 94 | 102 |

| | WV-20167/WV-36860, 6 mg/kg | | | | | |
|---|---|---|---|---|---|---|
| Day | animal11 | animal12 | animal13 | animal14 | animal15 | Mean |
| 1 | 92 | 102 | 109 | 119 | 92 | 103 |
| 8 | 18 | 18 | 19 | 18 | 16 | 18 |
| 15 | 33 | 38 | 39 | 30 | 34 | 35 |
| 22 | 57 | 78 | 72 | 55 | 66 | 66 |
| 29 | 72 | 80 | 85 | 71 | 81 | 78 |
| 36 | 90 | 119 | 104 | 96 | 100 | 102 |
| 43 | 94 | 98 | 115 | 105 | 106 | 104 |
| | WV-20170/WV-36807, 2 mg/kg | | | | | |
| Day | animal16 | animal17 | animal18 | animal19 | animal20 | Mean |
| 1 | 65 | 83 | 95 | 83 | 103 | 86 |
| 8 | 36 | 37 | 43 | 41 | 34 | 38 |
| 15 | 53 | 47 | 49 | 60 | 55 | 53 |
| 22 | 67 | 76 | 71 | 84 | 76 | 75 |
| 29 | 78 | 85 | 85 | 103 | 69 | 84 |
| 36 | 90 | 93 | 93 | 96 | 93 | 93 |
| 43 | 88 | 93 | 94 | 117 | 79 | 94 |
| | WV-20170/WV-36807, 6 mg/kg | | | | | |
| Day | animal21 | animal22 | animal23 | animal24 | animal25 | Mean |
| 1 | 108 | 129 | 100 | 105 | 107 | 110 |
| 8 | 9 | 11 | 4 | 8 | 15 | 10 |
| 15 | 17 | 19 | 9 | 19 | 29 | 19 |
| 22 | 39 | 40 | 18 | 39 | 58 | 39 |
| 29 | 67 | 69 | 41 | 56 | 79 | 62 |
| 36 | 90 | 94 | 61 | 84 | 110 | 88 |
| 43 | 108 | 114 | 80 | 90 | 107 | 100 |
| | WV-38708/WV-36807, 2 mg/kg | | | | | |
| Day | animal26 | animal27 | animal28 | animal29 | animal30 | Mean |
| 1 | 120 | 102 | 117 | 99 | 141 | 116 |
| 8 | 32 | 31 | 21 | 22 | 30 | 27 |
| 15 | 46 | 40 | 38 | 36 | 44 | 41 |
| 22 | 77 | 79 | 69 | 65 | 70 | 72 |
| 29 | 85 | 69 | 82 | 77 | 87 | 80 |
| 36 | 100 | 95 | 92 | 98 | 103 | 98 |
| 43 | 106 | 101 | 91 | 113 | 119 | 106 |
| | WV-38708/WV-36807, 6 mg/kg | | | | | |
| Day | animal31 | animal32 | animal33 | animal34 | animal35 | Mean |
| 1 | 102 | 103 | 107 | 105 | 117 | 107 |
| 8 | 7 | 4 | 4 | 5 | 7 | 6 |
| 15 | 13 | 9 | 7 | 11 | 12 | 10 |
| 22 | 27 | 20 | 19 | 27 | 30 | 25 |
| 29 | 48 | 38 | 40 | 52 | 63 | 48 |
| 36 | 77 | 66 | 66 | 72 | 83 | 73 |
| 43 | 82 | 89 | 77 | 115 | 94 | 91 |
| | WV-38706/WV-36807, 6 mg/kg | | | | | |
| Day | animal36 | animal37 | animal38 | animal39 | Animal40 | Mean |
| 1 | 114 | 129 | 142 | 104 | 111 | 120 |
| 8 | 123 | 93 | 100 | 106 | 102 | 105 |
| 15 | 127 | 111 | 119 | 104 | 100 | 112 |
| 22 | 130 | 100 | 101 | 103 | 109 | 109 |
| 29 | 121 | 91 | 126 | 102 | 105 | 109 |
| 36 | 117 | 135 | 108 | 106 | 109 | 115 |
| 43 | 120 | 114 | 120 | 132 | 102 | 117 |

Table 9. shows % mouse TTR mRNA remaining (at 300 and 100 pM siRNA treatment) relative to mouse HPRT control. N=2. N.D.: Not determined.

| | 300 pM | | | 100 pM | | |
|---|---|---|---|---|---|---|
| | % remaining | % remaining | | % remaining | % remaining | |
| | mRNA | mRNA | | mRNA | mRNA | |
| | (mTTR/ | (mTTR/ | | (mTTR/ | (mTTR/ | |
| siRNA | mHPRT)-1 | mHPRT)-2 | Mean | mHPRT)-1 | mHPRT)-2 | Mean |
| DSR-0101651 | 56.113 | 54.657 | 55.39 | 70.934 | 79.092 | 75.01 |
| DSR-0102524 | 46.386 | 34.305 | 40.35 | 76.224 | 57.865 | 67.04 |
| DSR-0103268 | 53.02 | 46.219 | 49.62 | 66.65 | 85.311 | 75.98 |
| DSR-0102161 | 57.793 | 45.721 | 51.76 | 91.94 | 60.264 | 76.10 |
| DSR-0103267 | 55.08 | 47.509 | 51.29 | 76.377 | 85.699 | 81.04 |
| DSR-0103266 | 47.401 | 41.727 | 44.56 | 75.259 | 62.845 | 69.05 |
| DSR-0103265 | 44.067 | 38.667 | 41.37 | 58.235 | 68.8 | 63.52 |
| DSR-0103264 | 52.212 | 48.956 | 50.58 | 78.578 | 67.927 | 73.25 |
| DSR-0103263 | 45.545 | 42.045 | 43.80 | 58.894 | 68.952 | 63.92 |
| DSR-0103262 | 53.611 | 41.367 | 47.49 | 79.49 | 60.584 | 70.04 |
| DSR-0103261 | 48.408 | 39.907 | 44.16 | 81.101 | 66.145 | 73.62 |
| DSR-0103260 | 58.72 | 47.415 | 53.07 | 87.199 | 62.272 | 74.74 |
| DSR-0103259 | 51.685 | 51.323 | 51.50 | 81.791 | 68.437 | 75.11 |
| DSR-0103258 | 47.667 | 38.217 | 42.94 | 68.023 | 65.279 | 66.65 |
| DSR-0103257 | 42.808 | 33.274 | 38.04 | 60.211 | 56.174 | 58.19 |
| DSR-0103256 | 53.223 | 46.577 | 49.90 | 73.002 | 70.513 | 71.76 |
| DSR-0103255 | 47.099 | 34.879 | 40.99 | 67.35 | 54.488 | 60.92 |
| DSR-0103254 | 58.712 | 49.158 | 53.94 | 82.3 | 67.993 | 75.15 |
| DSR-0103253 | 80.254 | 72.068 | 76.16 | 89.74 | 90.965 | 90.35 |
| DSR-0103252 | 83.201 | 80.517 | 81.86 | 95.172 | 102.116 | 98.64 |
| DSR-0103251 | 80.899 | 73.316 | 77.11 | 101.592 | 87.385 | 94.49 |
| DSR-0103250 | 95.286 | 95.152 | 95.22 | 106.407 | 109.407 | 107.91 |
| DSR-0103249 | 69.998 | 64.799 | 67.40 | 77.547 | 83.51 | 80.53 |
| DSR-0103248 | 88.732 | 86.846 | 87.79 | 96.76 | 104.801 | 100.78 |
| DSR-0103247 | 76.182 | 81.738 | 78.96 | 88.763 | 82.923 | 85.84 |
| DSR-0103246 | 98.911 | 92.585 | 95.75 | 110.514 | 113.56 | 112.04 |
| DSR-0103245 | 79.964 | 81.799 | 80.88 | 96.89 | 107.033 | 101.96 |
| DSR-0103244 | 82.89 | 81.533 | 82.21 | 98.283 | 94.37 | 96.33 |
| DSR-0103243 | 76.706 | 69.282 | 72.99 | 93.716 | 83.245 | 88.48 |
| DSR-0103242 | 70.084 | 64.651 | 67.37 | 97.212 | 85.997 | 91.60 |
| DSR-0103241 | 58.731 | 59.878 | 59.30 | 85.01 | 72.463 | 78.74 |
| DSR-0103240 | 81.878 | 79.308 | 80.59 | 96.758 | 100.935 | 98.85 |
| DSR-0103239 | 78.511 | 66.294 | 72.40 | 81.031 | 84.232 | 82.63 |
| DSR-0103238 | 70.707 | 66.602 | 68.65 | 95.922 | 87.921 | 91.92 |
| DSR-0103237 | 59.689 | 57.305 | 58.50 | 85.747 | 71.655 | 78.70 |
| DSR-0103236 | 58.868 | 59.182 | 59.03 | 81.563 | 77.43 | 79.50 |
| DSR-0103235 | 51.897 | 51.53 | 51.71 | 77.548 | 77.375 | 77.46 |
| DSR-0103234 | 51.616 | 43.004 | 47.31 | 67.075 | 75.288 | 71.18 |
| DSR-0103233 | 62.17 | 45.953 | 54.06 | 69.925 | 69.112 | 69.52 |
| DSR-0103232 | 62.285 | 51.385 | 56.84 | 70.226 | 84.397 | 77.31 |
| DSR-0103231 | 60.632 | 45.751 | 53.19 | 64.284 | 81.317 | 72.80 |
| DSR-0103230 | 60.696 | 53.028 | 56.86 | 74.074 | 81.77 | 77.92 |
| DSR-0103229 | 55.893 | 43.043 | 49.47 | 66.193 | 70.503 | 68.35 |
| DSR-0103228 | 61.225 | 50.887 | 56.06 | 61.807 | 78.262 | 70.03 |
| DSR-0103227 | 40.582 | 39.321 | 39.95 | 77.481 | 73.723 | 75.60 |

### Example 3. Improved Silencing Corresponds to Lower Thermal Stability

To investigate how the inclusion of PN linkages at backbone position +3 of the antisense strand impacts silencing, the thermal stability of siRNAs with and without these linkages were determined. For SSR-0102772, which is a stereopure siRNA that lacks PN linkages, the measured Tm was 69.6° C. Adding a PN linkage at position +3 in either an Sp (SSR-0104275) or Rp configuration (SSR-0104267) lowered Tm by ˜1° C. For WV-43775, which is a stereopure siRNA that lacks PN linkages and has a 2′-OMe on the 3′-side of the third backbone position, the measured Tm was 64.4° C. Adding a PN linkage at position +3 in either an Sp (WV-44453) or an Rp configuration (WV-46540) preserved or increased Tm compared with TTR-872. By contrast for SSR-0\, which has a 2′-F on the 3′-side of the third backbone position, the Tm decreased by ˜1° C. by the addition of a PN linkage in either an Sp (WV-47121) or Rp configuration (WV-47103). These data demonstrate that the inclusion of PN linkages can increase thermal instability, and increases in thermal instability are observed if the PN linkage is accompanied by an adjacent 2′-F ribose modification but not a 2′-OMe modification 3′ of the linkage.

To perform the thermal denaturation (Tm) experiments, equimolar amounts of sense and antisense strands were combined and annealed in 0.1×PBS (pH 7.2) to obtain a final concentration of 1 mM of each strand (3 ml). UV absorbance at 254 nm was recorded at intervals of 30 s as the temperature was raised from 15° C. to 95° C. at a rate of +0.5° C. per min, using a Cary Series UV-Vis spectrophotometer (Agilent Technologies). Absorbance was plotted against the temperature, and Tm values were calculated by taking the first derivative of each curve. The results are presented in Table 16.

Table 10. shows

| Passenger | Guide | Tm (Derivative) |
|---|---|---|
| WV-40362 | WV-20167 | 68.52 |
| WV-40363 | WV-20170 | 69.62 |
| WV-40363 | WV-41918 | 68.67 |
| WV-40363 | WV-41896 | 67.77 |
| WV-40363 | WV-38708 | 69.81 |
| WV-40363 | WV-38706 | 66.52 |
| WV-41828 | WV-49611 | 65.52 |
| WV-41828 | WV-49612 | 65.62 |
| WV-42080 | WV-51122 | 65.67 |
| WV-42080 | WV-47145 | 65.77 |
| WV-42080 | WV-48528 | 64.81 |
| SSR-0101630 | SSR-0105101 | 66.52 |
| SSR-0101630 | WV-49592 | 66.57 |
| SSR-0101695 | WV-49590 | 66.67 |
| — | — | — |
| WV-42080 | WV-43775 | 64.37 |
| WV-42080 | WV-44453 | 65.62 |
| WV-42080 | WV-46540 | 64.63 |
| WV-42080 | WV-47121 | 64.72 |
| WV-42080 | WV-47103 | 64.77 |
| WV-42080 | SSR-0106582 | 65.52 |

### Example 4. Provided Oligonucleotides and Compositions are Active In Vitro in Mouse Primary Hepatocytes

Various siRNAs for mouse APP were designed and constructed. A number of siRNAs were tested in vitro in mouse primary hepatocytes at one or a range of concentrations. Some siRNAs were also tested in mice (e.g., C57BL6 wild type mice).

Example protocol for in vitro determination of siRNA activity in mouse primary hepatocytes: For determination of siRNAs activity, siRNAs at specific concentration were gymnotically delivered to mouse primary hepatocytes plated at 96-well plates, with 10,000 cells/well. Following 48 hours treatment, total RNA was extracted using SV96 Total RNA Isolation kit (Promega). cDNA production from RNA samples were performed using High-Capacity cDNA Reverse Transcription kit (Thermo Fisher) following manufacturer's instructions and qPCR analysis performed in CFX System using iQ Multiplex Powermix (Bio-Rad). For mouse APP mRNA, the following qPCR assay were utilized: Thermofisher Taqman qPCR assay ID Mm01344172-ml. Mouse HPRT was used as normalizer (Forward 5′CAAACTTTGCTTTCCCTGGTT3′, Reverse 5′TGGCCTGTATCCAACACTTC3′, Probe 5′/5HEX/ACCAGCAAG/Zen/CTTGCAACCTTAACC/31ABkFQ/3′. mRNA knockdown levels were calculated as % mRNA remaining relative to mock treatment.

Table 11. shows 0% mouse APP mRNA remaining (at 300, 33.33 and 3.70 nM siRNA treatment) in primary mouse hepatocytes relative to mouse HPRT control. N=2. N.D.: Not determined.

| | 300 nM | | | 33.33 nM | | | 3.70 nM | | |
|---|---|---|---|---|---|---|---|---|---|
| | % | % | | % | % | | % | % | |
| | remaining | remaining | | remaining | remaining | | remaining | remaining | |
| | mRNA | mRNA | | mRNA | mRNA | | mRNA | mRNA | |
| | (mAPP/ | (mAPP/ | | (mAPP/ | (mAPP/ | | (mAPP/ | (mAPP/ | |
| siRNA | mHPRT)-1 | mHPRT)-2 | Mean | mHPRT)-1 | mHPRT)-2 | Mean | mHPRT)-1 | mHPRT)-2 | Mean |
| DSR- | 12.32 | 13.87 | 13.10 | 64.86 | 49 | 56.93 | 82.78 | 71.7 | 77.24 |
| 0102630 | | | | | | | | | |
| DSR- | 10.74 | 8.72 | 9.73 | 59.49 | 47.91 | 53.70 | 78.51 | 65.26 | 71.89 |
| 0102631 | | | | | | | | | |
| DSR- | 16.33 | 16.47 | 16.40 | 61.18 | 54.57 | 57.88 | 92.66 | 79.42 | 86.04 |
| 0102632 | | | | | | | | | |
| DSR- | 21.67 | 22.28 | 21.98 | 64.56 | 72.98 | 68.77 | 81.04 | 87.01 | 84.03 |
| 0102633 | | | | | | | | | |
| DSR- | 34.72 | 28.21 | 31.47 | 86.97 | 67.85 | 77.41 | 119.52 | 84.49 | 102.01 |
| 0102634 | | | | | | | | | |
| DSR- | 41.92 | 36.12 | 39.02 | 95.2 | 73.04 | 84.12 | 94.34 | 96 | 95.17 |
| 0102635 | | | | | | | | | |
| DSR- | 26.86 | 28.9 | 27.88 | 82.47 | 76.43 | 79.45 | 92.47 | 92.13 | 92.30 |
| 0102636 | | | | | | | | | |
| DSR- | 32.77 | 38.3 | 35.54 | 81.11 | 84.64 | 82.88 | 102.91 | 90.86 | 96.89 |
| 0102637 | | | | | | | | | |
| DSR- | 24.94 | 30.67 | 27.81 | 61.1 | 78.7 | 69.90 | 89.39 | 99.77 | 94.58 |
| 0102638 | | | | | | | | | |
| DSR- | 58.21 | 67.28 | 62.75 | 82.47 | 86.68 | 84.58 | 101.7 | 89.15 | 95.43 |
| 0102639 | | | | | | | | | |
| DSR- | 16.91 | 19.73 | 18.32 | 51.45 | 56.1 | 53.78 | 91.95 | 84.61 | 88.28 |
| 0102640 | | | | | | | | | |
| DSR- | 15.14 | 28.45 | 21.80 | 57.69 | 61.42 | 59.56 | 93.1 | 43.45 | 68.28 |
| 0102641 | | | | | | | | | |

### Example 5. Provided Oligonucleotides and Compositions are Active In Vitro in Mouse Neuro 2A Cells

Various siRNAs for mouse TTR were designed and constructed. A number of siRNAs were tested in vitro in mouse Neuro 2A cells at one or a range of concentrations. Some siRNAs were also tested in mice (e.g., C57BL6 wild type mice).

Example protocol for in vitro determination of siRNA activity in mouse Neuro2A cells: For determination of siRNAs activity, siRNAs at specific concentration were gymnotically delivered to mouse neuro2A cells plated at 96-well plates, with 25,000 cells/well. Following 48 hours treatment, total RNA was extracted using SV96 Total RNA Isolation kit (Promega). cDNA production from RNA samples were performed using High-Capacity cDNA Reverse Transcription kit (Thermo Fisher) following manufacturer's instructions and qPCR analysis performed in CFX System using iQ Multiplex Powermix (Bio-Rad). For mouse APP mRNA, the following qPCR assay were utilized: Thermofisher Taqman qPCR assay ID Mm01344172_m1. Mouse HPRT was used as normalizer (Forward 5′CAAACTTTGCTTTCCCTGGTT3′, Reverse 5′TGGCCTGTATCCAACACTTC3′, Probe 5′/5HEX/ACCAGCAAG/Zen/CTTGCAACCTTAACC/3IABkFQ/3′. mRNA knockdown levels were calculated as % mRNA remaining relative to mock treatment.

Table 12. shows % mouse APP mRNA remaining (at 5, 1 and 0.3 uM siRNA treatment) in mouse Neuro2A cells relative to mouse HPRT control. N=2. N.D.: Not determined.

| | 5 uM | | | 1 uM | | | 0.3 uM | | |
|---|---|---|---|---|---|---|---|---|---|
| | % | % | | % | % | | % | % | |
| | remaining | remaining | | remaining | remaining | | remaining | remaining | |
| | mRNA | mRNA | | mRNA | mRNA | | mRNA | mRNA | |
| | (mAPP/ | (mAPP/ | | (mAPP/ | (mAPP/ | | (mAPP/ | (mAPP/ | |
| siRNA | mHPRT)-1 | mHPRT)-2 | Mean | mHPRT)-1 | mHPRT)-2 | Mean | mHPRT)-1 | mHPRT)-2 | Mean |
| DSR- | 62.52 | 70.91 | 66.715 | 79.8 | 76.37 | 78.085 | 82.26 | 80.53 | 81.395 |
| 0102630 | | | | | | | | | |
| DSR- | 44.58 | 54.09 | 49.335 | 60.98 | 65.1 | 63.04 | 69.2 | 76.51 | 72.855 |
| 0102631 | | | | | | | | | |
| DSR- | 38.46 | 45.4 | 41.93 | 50.52 | 59.57 | 55.045 | 64.93 | 70.54 | 67.735 |
| 0102632 | | | | | | | | | |
| DSR- | 40.55 | 48.76 | 44.655 | 53.95 | 63.91 | 58.93 | 71.39 | 73.08 | 72.235 |
| 0102633 | | | | | | | | | |
| DSR- | 61.54 | 73.98 | 67.76 | 74.59 | 86.23 | 80.41 | 83.67 | 92.22 | 87.945 |
| 0102634 | | | | | | | | | |
| DSR- | 59.37 | 73.75 | 66.56 | 75.53 | 83.09 | 79.31 | 82.91 | 93.59 | 88.25 |
| 0102635 | | | | | | | | | |
| DSR- | 39.73 | 43.97 | 41.85 | 51.03 | 59.09 | 55.06 | 63.95 | 70.45 | 67.2 |
| 0102636 | | | | | | | | | |
| DSR- | 46.79 | 50.85 | 48.82 | 61.37 | 75.94 | 68.655 | 70.35 | 81.95 | 76.15 |
| 0102637 | | | | | | | | | |
| DSR- | 35.91 | 41.66 | 38.785 | 50.49 | 58.78 | 54.635 | 63.09 | 71.4 | 67.245 |
| 0102638 | | | | | | | | | |
| DSR- | 60.24 | 67.39 | 63.815 | 70.34 | 80.46 | 75.4 | 75.55 | 90.75 | 83.15 |
| 0102639 | | | | | | | | | |
| DSR- | 52.5 | 50.43 | 51.465 | 68.41 | 64.26 | 66.335 | 78.86 | 74.75 | 76.805 |
| 0102640 | | | | | | | | | |
| DSR- | 50.78 | 45.85 | 48.315 | 69.79 | 56.48 | 63.135 | 80.11 | 68.27 | 74.19 |
| 0102641 | | | | | | | | | |

Table 13. shows % mouse APP mRNA remaining (at 5, 1 and 0.3 uM siRNA treatment) in mouse Neuro2A cells relative to mouse HPRT control. N=2. N.D.: Not determined.

| | 5 uM | | | 1 uM | | | 0.3 uM | | |
|---|---|---|---|---|---|---|---|---|---|
| | % | % | | % | % | | % | % | |
| | remaining | remaining | | remaining | remaining | | remaining | remaining | |
| | mRNA | mRNA | | mRNA | mRNA | | mRNA | mRNA | |
| | (mAPP/ | (mAPP/ | | (mAPP/ | (mAPP/ | | (mAPP/ | (mAPP/ | |
| siRNA | mHPRT)-1 | mHPRT)-2 | Mean | mHPRT)-1 | mHPRT)-2 | Mean | mHPRT)-1 | mHPRT)-2 | Mean |
| DSR- | 69.20 | 71.60 | 70.40 | 91.70 | 90.10 | 90.90 | 98.60 | 108.00 | 103.30 |
| 0102661 | | | | | | | | | |
| DSR- | 57.10 | 63.60 | 60.35 | 76.80 | 78.50 | 77.65 | 83.30 | 99.10 | 91.20 |
| 0102662 | | | | | | | | | |
| DSR- | 66.00 | 75.80 | 70.90 | 95.70 | 89.20 | 92.45 | 90.30 | 112.90 | 101.60 |
| 0102663 | | | | | | | | | |
| DSR- | 67.30 | 73.70 | 70.50 | 84.20 | 97.50 | 90.85 | 89.60 | 96.00 | 92.80 |
| 0102664 | | | | | | | | | |
| DSR- | 37.20 | 44.30 | 40.75 | 58.00 | 70.70 | 64.35 | 71.50 | 88.10 | 79.80 |
| 0102665 | | | | | | | | | |
| DSR- | 40.90 | 44.70 | 42.80 | 54.80 | 28.40 | 41.60 | 70.40 | 82.90 | 76.65 |
| 0102666 | | | | | | | | | |
| DSR- | 41.60 | 46.60 | 44.10 | 64.20 | 65.30 | 64.75 | 80.30 | 91.60 | 85.95 |
| 0102667 | | | | | | | | | |
| DSR- | 43.90 | 43.70 | 43.80 | 68.10 | 57.70 | 62.90 | 74.20 | 98.40 | 86.30 |
| 0102668 | | | | | | | | | |
| DSR- | 44.60 | 54.10 | 49.35 | 66.60 | 72.30 | 69.45 | 77.60 | 90.00 | 83.80 |
| 0102669 | | | | | | | | | |
| DSR- | 42.50 | 55.50 | 49.00 | 66.10 | 67.20 | 66.65 | 51.20 | 85.80 | 68.50 |
| 0102670 | | | | | | | | | |
| DSR- | 44.90 | 45.90 | 45.40 | 66.30 | 71.10 | 68.70 | 74.50 | 86.30 | 80.40 |
| 0102671 | | | | | | | | | |
| DSR- | 51.30 | 52.90 | 52.10 | 80.70 | 36.80 | 58.75 | 80.00 | 50.40 | 65.20 |
| 0102672 | | | | | | | | | |
| DSR- | 36.29 | 49.24 | 42.77 | 46.54 | 62.03 | 54.29 | 64.88 | 78.58 | 71.73 |
| 0102632 | | | | | | | | | |
| DSR- | 29.87 | 40.51 | 35.19 | 35.11 | 59.41 | 47.26 | 43.02 | 73.69 | 58.36 |
| 0102636 | | | | | | | | | |
| DSR- | 15.88 | 30.89 | 23.39 | 38.94 | 67.51 | 53.23 | 92.96 | 131.61 | 112.29 |
| 0102640 | | | | | | | | | |

### Example 6. Provided Oligonucleotides and Compositions are Active In Vitro in Human iCell GABA Neurons

Various siRNAs for mouse TTR were designed and constructed. A number of siRNAs were tested in vitro in human iCell GABA neurons at one or a range of concentrations. Some siRNAs were also tested in mice (e.g., C57BL6 wild type mice).

Example protocol for in vitro determination of siRNA activity in human iCell GABA neurons: For determination of siRNAs activity, siRNAs at specific concentration were gymnotically delivered to human iCell GABA neurons plated at 96-well plates, with 40,000 cells/well. Following 5 days treatment, total RNA was extracted using SV96 Total RNA Isolation kit (Promega). cDNA production from RNA samples were performed using High-Capacity cDNA Reverse Transcription kit (Thermo Fisher) following manufacturer's instructions and qPCR analysis performed in CFX System using iQ Multiplex Powermix (Bio-Rad). For human APP mRNA, the following qPCR assay were utilized: Thermofisher Taqman qPCR assay ID Hs00169098-ml. Human SFRS9 was used as normalizer (Forward, 5′-TGGAATATGCCCTGCGTAAA-3′; Reverse, 5′-TGGTGCTTCTCTCAGGATAAAC-3′, Probe, 5′-TGGATGACACCAAATTCCGCTCTCA-3′. mRNA knockdown levels were calculated as % mRNA remaining relative to mock treatment.

Table 14. shows % human APP mRNA remaining (at 5, 1 and 0.3 uM siRNA treatment) in iCell GABA neurons relative to human SFRS9 control. N=2. N.D.: Not determined.

| | 5 uM | | | 1 uM | | | 0.3 uM | | |
|---|---|---|---|---|---|---|---|---|---|
| | % | % | | % | % | | % | % | |
| | remaining | remaining | | remaining | remaining | | remaining | remaining | |
| | mRNA | mRNA | | mRNA | mRNA | | mRNA | mRNA | |
| | (hAPP/ | (hAPP/ | | (hAPP/ | (hAPP/ | | (hAPP/ | (hAPP/ | |
| siRNA | hSFRS9)-1 | hSFRS9)-2 | Mean | hSFRS9)-1 | hSFRS9)-2 | Mean | hSFRS9)-1 | hSFRS9)-2 | Mean |
| DSR- | 97.60 | 108.20 | 102.9 | 113.30 | 111.00 | 112.15 | 94.60 | 110.80 | 102.7 |
| 0102630 | | | | | | | | | |
| DSR- | 97.30 | 96.60 | 96.95 | 91.90 | 97.90 | 94.9 | 94.20 | 97.10 | 95.65 |
| 0102631 | | | | | | | | | |
| DSR- | 31.70 | 30.20 | 30.95 | 62.90 | 72.90 | 67.9 | 82.40 | 90.30 | 86.35 |
| 0102632 | | | | | | | | | |
| DSR- | 41.00 | 55.90 | 48.45 | 77.10 | 84.90 | 81 | 80.10 | 79.70 | 79.9 |
| 0102633 | | | | | | | | | |
| DSR- | 51.40 | 55.60 | 53.5 | 73.80 | 79.00 | 76.4 | 89.40 | 77.70 | 83.55 |
| 0102634 | | | | | | | | | |
| DSR- | 53.70 | 68.60 | 61.15 | 65.80 | 96.10 | 80.95 | 88.50 | 96.00 | 92.25 |
| 0102635 | | | | | | | | | |
| DSR- | 25.50 | 28.30 | 26.9 | 53.20 | 74.80 | 64 | 85.50 | 77.00 | 81.25 |
| 0102636 | | | | | | | | | |
| DSR- | 44.90 | 57.80 | 51.35 | 71.50 | 71.90 | 71.7 | 82.90 | 76.10 | 79.5 |
| 0102637 | | | | | | | | | |
| DSR- | 73.00 | 36.00 | 54.5 | 68.10 | 82.70 | 75.4 | 128.30 | 71.50 | 99.9 |
| 0102638 | | | | | | | | | |
| DSR- | N.D | 33.90 | 16.95 | 64.80 | 56.00 | 60.4 | 75.70 | 73.10 | 74.4 |
| 0102639 | | | | | | | | | |
| DSR- | 44.60 | 42.20 | 43.4 | 82.30 | 88.90 | 85.6 | 82.40 | 103.50 | 92.95 |
| 0102640 | | | | | | | | | |
| DSR- | 58.80 | 65.30 | 62.05 | 98.20 | 74.50 | 86.35 | 116.20 | 89.00 | 102.6 |
| 0102641 | | | | | | | | | |

Table 15. shows 0% human APP mRNA remaining (at 5, 1.5 and 0.5 uM siRNA treatment) in iCell GABA neurons relative to human SFRS9 control. N=2. N.D.: Not determined.

| | 5 uM | | | 1.5 uM | | | 0.5 uM | | |
|---|---|---|---|---|---|---|---|---|---|
| | % | % | | % | % | | % | % | |
| | remaining | remaining | | remaining | remaining | | remaining | remaining | |
| | mRNA | mRNA | | mRNA | mRNA | | mRNA | mRNA | |
| | (hAPP/ | (hAPP/ | | (hAPP/ | (hAPP/ | | (hAPP/ | (hAPP/ | |
| siRNA | hSFRS9)-1 | hSFRS9)-2 | Mean | hSFRS9)-1 | hSFRS9)-2 | Mean | hSFRS9)-1 | hSFRS9)-2 | Mean |
| DSR- | 43.50 | 102.80 | 73.15 | 41.20 | 54.00 | 47.60 | 62.50 | 0.00 | 31.25 |
| 0102663 | | | | | | | | | |
| DSR- | 40.90 | 46.10 | 43.50 | 39.00 | 53.90 | 46.45 | 55.30 | 65.30 | 60.30 |
| 0102664 | | | | | | | | | |
| DSR- | 43.30 | 38.90 | 41.10 | N.D | 45.20 | 22.60 | 66.70 | 86.80 | 76.75 |
| 0102665 | | | | | | | | | |
| DSR- | 127.20 | 33.00 | 80.10 | 59.20 | 69.50 | 64.35 | 35.40 | 85.00 | 60.20 |
| 0102667 | | | | | | | | | |
| DSR- | N.D | 78.00 | 39.00 | 77.20 | 72.20 | 74.70 | 61.70 | 77.50 | 69.60 |
| 0102668 | | | | | | | | | |
| DSR- | 56.30 | 60.30 | 58.30 | 95.90 | 72.10 | 84.00 | 80.60 | 108.30 | 94.45 |
| 0102669 | | | | | | | | | |
| DSR- | 46.50 | 62.00 | 54.25 | 37.30 | 67.10 | 52.20 | 92.00 | 93.70 | 92.85 |
| 0102670 | | | | | | | | | |
| DSR- | 67.50 | 132.70 | 100.10 | 160.40 | 137.60 | 149.00 | 147.20 | 149.70 | 148.45 |
| 0102672 | | | | | | | | | |
| DSR- | 16.40 | 20.30 | 18.35 | 23.80 | 41.80 | 32.80 | 27.10 | 155.70 | 91.40 |
| 0102632 | | | | | | | | | |
| DSR- | 19.10 | 22.10 | 20.60 | 28.70 | 34.10 | 31.40 | 37.70 | 59.50 | 48.60 |
| 0102636 | | | | | | | | | |
| DSR- | 48.30 | 54.60 | 51.45 | 59.30 | 61.10 | 60.20 | 125.00 | 100.90 | 112.95 |
| 0102640 | | | | | | | | | |

Table 16. shows % human APP mRNA remaining (at 5 uM siRNA treatment) in iCell GABA neurons relative to human SFRS9 control. N=2. N.D. Not determined.

5 uM

| | % remaining | % remaining | |
|---|---|---|---|
| | mRNA (hAPP/ | mRNA (hAPP/ | |
| siRNA | hSFRS9)-1 | hSFRS9)-2 | Mean |
| DSR-0102661 | 48.40 | 41.50 | 44.95 |
| DSR-0102662 | 73.10 | 54.10 | 63.60 |
| DSR-0102663 | 67.40 | 59.80 | 63.60 |
| DSR-0102664 | 62.50 | 62.10 | 62.30 |
| DSR-0102665 | 27.70 | 29.30 | 28.50 |
| DSR-0102666 | 47.60 | 46.10 | 46.85 |
| DSR-0102667 | 30.00 | 34.60 | 32.30 |
| DSR-0102668 | 33.90 | 34.20 | 34.05 |
| DSR-0102669 | 32.20 | 35.10 | 33.65 |
| DSR-0102670 | 54.00 | 48.70 | 51.35 |
| DSR-0102671 | 54.90 | 60.80 | 57.85 |
| DSR-0102672 | 70.70 | 77.90 | 74.30 |
| DSR-0102632 | 25.00 | 25.50 | 25.25 |
| DSR-0102636 | 23.00 | 22.50 | 22.75 |
| DSR-0102640 | 46.80 | 53.30 | 50.05 |
| DSR-0103338 | 151.30 | 65.30 | 108.30 |
| DSR-0103340 | 64.30 | 54.30 | 59.30 |
| DSR-0103190 | 85.70 | 76.00 | 80.85 |
| DSR-0103137 | 81.30 | 118.10 | 99.70 |
| DSR-0103339 | 79.70 | 71.00 | 75.35 |
| DSR-0103337 | 81.10 | 99.60 | 90.35 |

Table 17. shows % human APP mRNA remaining (at 3 uM siRNA treatment) in iCell GABA neurons relative to human SFRS9 control. N=2. N.D. Not determined.

3 uM

| | % remaining | % remaining | |
|---|---|---|---|
| | mRNA (hAPP/ | mRNA (hAPP/ | |
| siRNA | hSFRS9)-1 | hSFRS9)-2 | Mean |
| DSR-0103190 | 47.40 | 44.30 | 45.85 |
| DSR-0103137 | 77.30 | 76.80 | 77.05 |
| DSR-0102636 | 17.50 | 25.10 | 21.30 |
| DSR-0103185 | 17.60 | 27.80 | 22.70 |
| DSR-0103179 | 21.30 | 24.50 | 22.90 |
| DSR-0103141 | 21.80 | 28.40 | 25.10 |
| DSR-0103192 | 27.10 | 35.90 | 31.50 |
| DSR-0103180 | 18.70 | 25.20 | 21.95 |
| DSR-0103171 | 15.90 | 15.10 | 15.50 |
| DSR-0103162 | 33.90 | 43.70 | 38.80 |
| DSR-0103163 | 26.80 | 28.00 | 27.40 |
| DSR-0103172 | 33.60 | 42.60 | 38.10 |
| DSR-0103148 | 24.20 | 21.30 | 22.75 |
| DSR-0103154 | 18.60 | 19.60 | 19.10 |
| DSR-0103173 | 20.60 | 21.40 | 21.00 |
| DSR-0103149 | 28.00 | 28.70 | 28.35 |
| DSR-0103155 | 19.20 | 26.80 | 23.00 |
| DSR-0103164 | 12.80 | 11.70 | 12.25 |
| DSR-0103142 | 15.10 | 15.10 | 15.10 |
| DSR-0103138 | 14.00 | 15.00 | 14.50 |
| DSR-0103168 | 15.60 | 15.40 | 15.50 |
| DSR-0103160 | 15.20 | 19.10 | 17.15 |
| DSR-0103161 | 14.20 | 18.00 | 16.10 |
| DSR-0103139 | 14.80 | 15.50 | 15.15 |
| DSR-0103169 | 14.50 | 24.20 | 19.35 |
| DSR-0103153 | 23.20 | 18.60 | 20.90 |
| DSR-0103170 | 28.80 | 32.40 | 30.60 |
| DSR-0103184 | 18.20 | 21.10 | 19.65 |
| DSR-0103147 | 18.90 | 14.70 | 16.80 |
| DSR-0103140 | 22.40 | 21.30 | 21.85 |
| DSR-0103191 | 22.30 | 31.50 | 26.90 |
| DSR-0103178 | 17.70 | 19.50 | 18.60 |

Table 18. shows % human APP mRNA remaining (at 3 uM siRNA treatment) in iCell GABA neurons relative to human SFRS9 control. N=2. N.D. Not determined.

3 uM

| | % remaining | % remaining | |
|---|---|---|---|
| | mRNA (hAPP/ | mRNA (hAPP/ | |
| siRNA | hSFRS9)-1 | hSFRS9)-2 | Mean |
| DSR-0103190 | 47.40 | 44.30 | 45.85 |
| DSR-0103137 | 77.30 | 76.80 | 77.05 |
| DSR-0103189 | 19.40 | 21.70 | 20.55 |
| DSR-0103144 | 20.00 | 21.90 | 20.95 |
| DSR-0103176 | 21.40 | 21.30 | 21.35 |
| DSR-0103145 | 19.90 | 26.20 | 23.05 |
| DSR-0103157 | 20.10 | 28.00 | 24.05 |
| DSR-0103152 | 18.40 | 26.20 | 22.30 |
| DSR-0103167 | 20.00 | 25.50 | 22.75 |
| DSR-0103158 | 31.30 | 37.90 | 34.60 |
| DSR-0103187 | 24.20 | 22.50 | 23.35 |
| DSR-0102665 | 29.70 | 33.90 | 31.80 |
| DSR-0103146 | 22.70 | 29.40 | 26.05 |
| DSR-0103177 | 19.50 | 22.00 | 20.75 |
| DSR-0103135 | 21.20 | 21.20 | 21.20 |
| DSR-0103136 | 28.10 | 36.20 | 32.15 |
| DSR-0103183 | 27.70 | 35.10 | 31.40 |
| DSR-0103188 | 17.70 | 18.80 | 18.25 |
| DSR-0103159 | 19.30 | 21.20 | 20.25 |
| DSR-0103165 | 20.90 | 20.70 | 20.80 |
| DSR-0103156 | 24.10 | 20.90 | 22.50 |
| DSR-0103150 | 18.50 | 17.20 | 17.85 |
| DSR-0103133 | 17.60 | 18.20 | 17.90 |
| DSR-0103143 | 19.90 | 24.20 | 22.05 |
| DSR-0103134 | 28.30 | 31.90 | 30.10 |
| DSR-0103186 | 25.80 | 21.70 | 23.75 |
| DSR-0103174 | 21.00 | 24.00 | 22.50 |
| DSR-0103166 | 23.30 | 22.60 | 22.95 |
| DSR-0103181 | 25.70 | 19.30 | 22.50 |
| DSR-0103175 | 24.30 | 22.60 | 23.45 |
| DSR-0103182 | 36.50 | 35.40 | 35.95 |
| DSR-0103151 | 26.50 | 26.40 | 26.45 |

Table 19. shows 0% human APP mRNA remaining (at 6, 0.6, and 0.03 uM siRNA treatment) in iCell GABA neurons relative to human SFRS9 control. N=2. N.D.: Not determined.

| | 6 uM | | | 0.6 uM | | | 0.06 uM | | |
|---|---|---|---|---|---|---|---|---|---|
| | % | % | | % | % | | % | % | |
| | remaining | remaining | | remaining | remaining | | remaining | remaining | |
| | mRNA | mRNA | | mRNA | mRNA | | mRNA | mRNA | |
| | (hAPP/ | (hAPP/ | | (hAPP/ | (hAPP/ | | (hAPP/ | (hAPP/ | |
| siRNA | hSFRS9)-1 | hSFRS9)-2 | Mean | hSFRS9)-1 | hSFRS9)-2 | Mean | hSFRS9)-1 | hSFRS9)-2 | Mean |
| DSR- | 45.00 | 61.00 | 53.00 | 69.00 | 88.50 | 78.75 | 91.30 | 95.60 | 93.45 |
| 0103190 | | | | | | | | | |
| DSR- | 11.10 | 9.40 | 10.25 | 35.80 | 39.30 | 37.55 | 99.10 | 82.20 | 90.65 |
| 0103164 | | | | | | | | | |
| DSR- | 12.30 | 10.50 | 11.40 | 45.40 | 45.50 | 45.45 | 88.90 | 88.50 | 88.70 |
| 0103142 | | | | | | | | | |
| DSR- | 12.00 | 10.70 | 11.35 | 42.10 | 38.80 | 40.45 | 85.80 | 86.50 | 86.15 |
| 0103138 | | | | | | | | | |
| DSR- | 12.80 | 13.40 | 13.10 | 39.50 | 46.70 | 43.10 | 78.60 | 90.50 | 84.55 |
| 0103168 | | | | | | | | | |
| DSR- | 12.60 | 15.20 | 13.90 | 52.60 | 54.10 | 53.35 | 87.50 | 113.90 | 100.70 |
| 0103160 | | | | | | | | | |
| DSR- | 10.80 | 9.60 | 10.20 | 36.90 | 39.40 | 38.15 | 79.20 | 85.90 | 82.55 |
| 0103161 | | | | | | | | | |
| DSR- | 14.10 | 16.00 | 15.05 | 42.90 | 44.80 | 43.85 | 111.10 | 102.40 | 106.75 |
| 0103139 | | | | | | | | | |
| DSR- | 15.60 | 17.80 | 16.70 | 45.20 | 60.00 | 52.60 | 70.40 | 80.40 | 75.40 |
| 0103169 | | | | | | | | | |
| DSR- | 17.50 | 15.10 | 16.30 | 65.70 | 66.30 | 66.00 | 101.50 | 83.40 | 92.45 |
| 0103153 | | | | | | | | | |
| DSR- | 17.80 | 14.70 | 16.25 | 67.30 | 69.30 | 68.30 | 73.20 | 93.30 | 83.25 |
| 0103170 | | | | | | | | | |
| DSR- | 15.10 | 13.30 | 14.20 | 49.70 | 47.40 | 48.55 | 90.80 | 91.30 | 91.05 |
| 0103184 | | | | | | | | | |
| DSR- | 11.00 | 11.20 | 11.10 | 38.60 | 39.10 | 38.85 | 61.90 | 78.20 | 70.05 |
| 0103147 | | | | | | | | | |
| DSR- | 15.40 | 14.70 | 15.05 | 33.00 | 38.40 | 35.70 | 74.10 | 100.60 | 87.35 |
| 0103140 | | | | | | | | | |
| DSR- | 20.00 | 20.20 | 20.10 | 47.20 | 49.00 | 48.10 | 76.50 | 80.00 | 78.25 |
| 0103191 | | | | | | | | | |
| DSR- | 18.30 | 18.70 | 18.50 | 62.50 | 74.70 | 68.60 | 112.30 | 110.80 | 111.55 |
| 0103178 | | | | | | | | | |

Table 20. shows 00 human APP mRNA remaining (at 3 and 1 uM siRNA treatment) in iCell GABA neurons relative to human SFRS9 control. N=2. N.D. Not determined.

| | 3 uM | | | 1 uM | | |
|---|---|---|---|---|---|---|
| | % | % | | % | % | |
| | remaining | remaining | | remaining | remaining | |
| | mRNA | mRNA | | mRNA | mRNA | |
| | (hAPP/ | (hAPP/ | | (hAPP/ | (hAPP/ | |
| siRNA | hSFRS9)-1 | hSFRS9)-2 | Mean | hSFRS9)-1 | hSFRS9)-2 | Mean |
| DSR-0103190 | 54.40 | 56.70 | 55.55 | 58.00 | 86.50 | 72.25 |
| DSR-0103161 | 14.50 | 15.20 | 14.85 | 37.00 | 45.40 | 41.20 |
| DSR-0103219 | 19.10 | 17.50 | 18.30 | 33.90 | 36.40 | 35.15 |
| DSR-0103212 | 13.90 | 17.70 | 15.80 | 35.20 | 39.60 | 37.40 |
| DSR-0103201 | 16.80 | 16.70 | 16.75 | 33.20 | 43.90 | 38.55 |
| DSR-0103226 | 13.40 | 15.90 | 14.65 | 27.30 | 33.00 | 30.15 |
| DSR-0103202 | 16.70 | 20.50 | 18.60 | 33.00 | 48.00 | 40.50 |
| DSR-0103207 | 15.30 | 16.10 | 15.70 | 37.80 | 30.70 | 34.25 |
| DSR-0103203 | 27.10 | 29.00 | 28.05 | 42.30 | 62.40 | 52.35 |
| DSR-0103213 | 26.20 | 31.20 | 28.70 | 52.80 | 70.40 | 61.60 |
| DSR-0103214 | 22.60 | 24.10 | 23.35 | 41.30 | 45.30 | 43.30 |
| DSR-0103208 | 24.20 | 30.30 | 27.25 | 47.10 | 56.30 | 51.70 |
| DSR-0103220 | 22.50 | 19.40 | 20.95 | 29.90 | 32.30 | 31.10 |
| DSR-0103209 | 22.00 | 25.30 | 23.65 | 54.30 | 68.20 | 61.25 |
| DSR-0103204 | 17.70 | 24.70 | 21.20 | 44.90 | 46.60 | 45.75 |
| DSR-0103215 | 20.20 | 24.00 | 22.10 | 43.70 | 62.40 | 53.05 |

Table 21. shows 00 human APP mRNA remaining (at 3 and 1 uM siRNA treatment) in iCell GABA neurons relative to human SFRS9 control. N=2. N.D. Not determined.

| | 3 uM | | | 1 uM | | |
|---|---|---|---|---|---|---|
| | % | % | | % | % | |
| | remaining | remaining | | remaining | remaining | |
| | mRNA | mRNA | | mRNA | mRNA | |
| | (hAPP/ | (hAPP/ | | (hAPP/ | (hAPP/ | |
| siRNA | hSFRS9)-1 | hSFRS9)-2 | Mean | hSFRS9)-1 | hSFRS9)-2 | Mean |
| DSR-0103190 | 70.20 | 87.40 | 78.80 | 96.40 | 74.30 | 85.35 |
| DSR-0103164 | 13.60 | 16.40 | 15.00 | 27.10 | 30.10 | 28.60 |
| DSR-0103161 | 7.10 | 26.30 | 16.70 | 36.80 | 40.40 | 38.60 |
| DSR-0103221 | 14.20 | 14.20 | 14.20 | 24.70 | 29.30 | 27.00 |
| DSR-0103222 | 11.80 | 18.90 | 15.35 | 28.40 | 48.50 | 38.45 |
| DSR-0103199 | 12.20 | 30.50 | 21.35 | 21.70 | 24.10 | 22.90 |
| DSR-0103216 | 15.70 | 16.40 | 16.05 | 25.00 | 28.10 | 26.55 |
| DSR-0103205 | 10.40 | 15.70 | 13.05 | 35.70 | 32.40 | 34.05 |
| DSR-0103200 | 12.10 | 19.10 | 15.60 | 25.50 | 32.40 | 28.95 |

Table 22. shows % human APP mRNA remaining (at 3 and 1 uM siRNA treatment) in iCell GABA neurons relative to human SFRS9 control. N=2 N.D.: Not determined.

| | 3 uM | | | 1 uM | | |
|---|---|---|---|---|---|---|
| | % remaining | % remaining | | % remaining | % remaining | |
| | mRNA | mRNA | | mRNA | mRNA | |
| | (hAPP/ | (hAPP/ | | (hAPP/ | (hAPP/ | |
| siRNA | hSFRS9)-1 | hSFRS9)-2 | Mean | hSFRS9)-1 | hSFRS9)-2 | Mean |
| DSR-0103190 | 61.00 | 60.40 | 60.70 | 70.70 | 87.30 | 79.00 |
| DSR-0103164 | 12.90 | 14.40 | 13.65 | 27.20 | 27.90 | 27.55 |
| DSR-0103210 | 53.20 | 50.00 | 51.60 | 60.90 | 74.20 | 67.55 |
| DSR-0103211 | 60.70 | 64.40 | 62.55 | 88.90 | 73.90 | 81.40 |
| DSR-0103206 | 13.70 | 17.40 | 15.55 | 32.10 | 46.90 | 39.50 |
| DSR-0103217 | 27.90 | 35.10 | 31.50 | 66.10 | 61.90 | 64.00 |
| DSR-0103223 | 14.90 | 17.00 | 15.95 | 30.70 | 45.60 | 38.15 |
| DSR-0103224 | 28.40 | 78.60 | 53.50 | 61.20 | 66.20 | 63.70 |
| DSR-0103225 | 16.70 | 22.20 | 19.45 | 47.20 | 63.20 | 55.20 |
| DSR-0103218 | 26.30 | 33.50 | 29.90 | 79.00 | 64.60 | 71.80 |

Table 23. shows % IC50 of knocking down human APP mRNA in human iCell GABA neurons.

| siRNA | IC50 (uM) | 95% CI |
|---|---|---|
| DSR-0103164 | 0.2700 | 0.1474 to 0.4916 |
| DSR-0103275 | 0.3049 | 0.1615 to 0.5520 |
| DSR-0103276 | 0.2856 | 0.1768 to 0.4624 |
| DSR-0103199 | 0.2226 | 0.1584 to 0.3131 |
| DSR-0103216 | 0.2762 | 0.1250 to 0.5945 |
| DSR-0103206 | 0.7064 | 0.3981 to 1.271 |
| DSR-0103223 | 1.051 | 0.5869 to 1.932 |
| DSR-0103225 | 1.902 | 0.7058 to 6.344 |

Table 24. shows % human APP mRNA remaining (at 6, 0.6, and 0.2 uM siRNA treatment) in iCell GABA neurons relative to human SFRS9 control. N=2. N.D.: Not determined.

| | 6 uM | | | 0.6 uM | | | 0.2 uM | | |
|---|---|---|---|---|---|---|---|---|---|
| | % remaining | % remaining | | % remaining | % remaining | | % remaining | % remaining | |
| | mRNA | mRNA | | mRNA | mRNA | | mRNA | mRNA | |
| | (hAPP/ | (hAPP/ | | (hAPP/ | (hAPP/ | | (hAPP/ | (hAPP/ | |
| siRNA | hSFRS9)-1 | hSFRS9)-2 | Mean | hSFRS9)-1 | hSFRS9)-2 | Mean | hSFRS9)-1 | hSFRS9)-2 | Mean |
| DSR-0103275 | 10.70 | 12.50 | 11.60 | 34.40 | 38.40 | 36.40 | 53.30 | 62.70 | 58.00 |
| DSR-0103325 | 15.00 | 18.30 | 16.65 | 43.60 | 50.30 | 46.95 | 67.00 | 74.60 | 70.80 |
| DSR-0103326 | 10.00 | 11.80 | 10.90 | 35.90 | 35.50 | 35.70 | 56.40 | 55.80 | 56.10 |
| DSR-0103319 | 9.40 | 12.40 | 10.90 | 31.10 | 40.50 | 35.80 | 59.70 | 60.90 | 60.30 |
| DSR-0103320 | 9.90 | 13.30 | 11.60 | 41.10 | 32.00 | 36.55 | 61.50 | 66.30 | 63.90 |
| DSR-0103321 | 16.90 | 19.50 | 18.20 | 55.50 | 53.70 | 54.60 | 77.70 | 70.60 | 74.15 |
| DSR-0103322 | 13.40 | 17.30 | 15.35 | 47.80 | 49.40 | 48.60 | 72.10 | 71.10 | 71.60 |
| DSR-0103323 | 16.90 | 17.60 | 17.25 | 48.50 | 60.20 | 54.35 | 74.50 | 77.70 | 76.10 |
| DSR-0103324 | 85.10 | 91.10 | 88.10 | 93.00 | 98.60 | 95.80 | 93.30 | 108.90 | 101.10 |

### Example 7. Provided Oligonucleotides and Compositions are Active In Vivo

All animal procedures were performed under IACUC guidelines. Male 8-9 weeks of age C57BL/6 mice underwent surgery for the implantation of intracerebroventricular cannulas. At 10-11 weeks of age the same mice were dosed 100 mg/kg on Day 0 by intracerebroventricular injection. At day 14 animals in groups 1-6 were euthanized by CO₂ asphyxiation followed by thoracotomy. After cardiac perfusion with PBS brain samples were taken from both hemispheres (cortex, striatum, hippocampus, cerebellum, and brain stem) and flash frozen on dry ice. At day 56 animals in groups 7-12 were euthanized by CO₂ asphyxiation followed by thoracotomy and terminal blood collection. After cardiac perfusion with PBS brain samples were taken from both hemispheres (cortex, striatum, hippocampus, cerebellum, brain stem, and spinal cord) and flash frozen on dry ice. Two animals from each group had the right hemisphere of their brain placed in buffered 10% formalin. Total RNA from different CNS regions was extracted using RNeasy 96 kit (Qiagen), after tissue lysis with TRIzol and chloroform. cDNA production from RNA samples were performed using High-Capacity cDNA Reverse Transcription kit (Thermo Fisher) following manufacturer's instructions and qPCR analysis performed in CFX System using iQ Multiplex Powermix (Bio-Rad For mouse APP mRNA, the following qPCR assay were utilized: Thermofisher Taqman qPCR assay ID Mm01344172_m1. Mouse HPRT was used as normalizer (Forward 5′CAAACTTTGCTTTCCCTGGTT3′, Reverse 5′TGGCCTGTATCCAACACTTC3′, Probe 5′/5HEX/ACCAGCAAG/Zen/CTTGCAACCTTAACC/3IABkFQ/3′. cDNA production from RNA samples were performed using High-Capacity cDNA Reverse Transcription kit (Thermo Fisher) following manufacturer's instructions and qPCR analysis performed in CFX System using iQ Multiplex Powermix (Bio-Rad For neurotoxicity markers, the following qPCR assays were utilized: Thermofisher Taqman qPCR assay ID for Calb1: Mm00486647_m1, Grid2: Mm00515047_m1, Pcp2: Mm00435514_m1, Gfap: Mm01253033_m1, Aif1: Mm00479862_g1. Thermofisher Taqman qPCR assay ID Mouse Tubb3 which was used as normalizer: Mm00727586_s1.

Oligonucleotide accumulation in CNS regions was determined by sandwich ELISA.

To evaluate the safety of siRNAs used, serum NfL levels were measured at 4-, 6-, and 8-weeks following administration. Monoclonal antibody UD1 (Uman Diagnotics) was covalently coupled to MagPlex-C Microspheres #12 (MC10012-01, Luminex) using xMAP Antibody Coupling Kit (#40-50016, Luminex). Serum samples from mice were diluted as 1:4 with Specimen Diluent Buffer (Quidel #A3670) and incubated with UD1 coupled microspheres (2500 beads/well) in a 96-well plate for 18-20 hours at 4 C on an orbital plated shaker. Following three washes with Wash Buffer (EMD Millipore #L-WB), the wells were subjected to an incubation step with 25 μL of Detection antibody (1:500, UD3, Uman Diagnostics) on a plate shaker for 2 hours at ambient temperature. 25 μL of Streptavidin-Phycoerthrin (EMD Millipore #LB-SAPE10) was introduced into each well, followed by an incubation period of 30 minutes with continuous agitation. Following three rounds of washing, 150 microliters of Sheath Fluid PLUS (EMD Millipore #40-50021) was introduced, and subsequently, the beads were resuspended on a plate shaker for a duration of 5 minutes. The fluorescence intensity signal of the beads was measured using FLEXMAP 3D (Luminex) instrument with xPONENT software (Luminex). The analyses were conducted in duplicate to ensure accuracy and reliability. The standard NfL protein (Progen #RPC20469) was used to prepare five standards at the concentration from 30 to 200,000 pg/ml in Assay buffer (EMD Millipore #L-AB) Unspiked assay buffer was used as blank. Standard curves were fitted using 5-Parameter Logistic regression analysis by XPONENT software. Average NfL concentration of each group was calculated and plotted using GraphPad Prism. Minimal changes in the serum levels of NfL were observed in the mice treated with siRNA, in comparison to the group treated with PBS. There was no statistically significant difference observed between the groups.

Cerebellum total RNA extracted from mice treated with PBS, DSR-0103199, and DSR-0103299 (3 animals from each group) were RNA-sequenced and Fastq's were obtained from Novogene. nf-core/rnaseq (version 3.10.1) pipeline was used to process the data, with the specific packages following. Adapters were trimmed using Trim Galore!(Version 0.6.7 & cutadapt version 3.4) then aligned using STAR (version 2.7.9a) and transcript counts quantified using Salmon (version 1.9.0). Aligned bam files were processed using R (version 4.3.0) and differential expression results calculated using DEseq2 (version 1.40.2) and plotted using EnhancedVolcano (version 1.18.0) with cutoffs greater than |log 2foldChange|>1 and padj (Benjamini-Hochberg) less than 0.01.

Table 25. shows % mouse APP mRNA remaining relative to PBS control in the cortex 14 days post dosing. N=7. N.D.: Not determined.

| animal | % remaining of mAPP mRNA | | | | | |
|---|---|---|---|---|---|---|
| No. | PBS | DSR-0103199 | DSR-0103216 | DSR-0103299 | DSR-0103300 | DSR-0103301 |
| 1 | 93.40 | 44.60 | 40.40 | 15.60 | 33.90 | 37.60 |
| 2 | 84.40 | 9.40 | 22.60 | 49.30 | 46.20 | 16.20 |
| 3 | 98.10 | 17.10 | 22.40 | 6.50 | 15.50 | 31.90 |
| 4 | 101.50 | 74.10 | 16.20 | 19.70 | 38.30 | 16.90 |
| 5 | 105.50 | 35.30 | 78.10 | 24.90 | 55.60 | 19.40 |
| 6 | 116.90 | 29.20 | 30.70 | 29.50 | 23.20 | 19.40 |
| 7 | 100.20 | 16.50 | 26.60 | 38.30 | 20.10 | 19.80 |
| Mean | 100.00 | 32.31 | 33.86 | 26.26 | 33.26 | 23.03 |

Table 26. shows % mouse APP mRNA remaining relative to PBS control in the cortex 56 days post dosing. N=7. N.D.: Not determined.

| animal | % remaining of mAPP mRNA | | | | | |
|---|---|---|---|---|---|---|
| No. | PBS | DSR-0103199 | DSR-0103216 | DSR-0103299 | DSR-0103300 | DSR-0103301 |
| 1 | 114.3 | 15.5 | 27.1 | 35.4 | 35.2 | 9.9 |
| 2 | 105.7 | 9.4 | 17.4 | 21.3 | 21.9 | 17.1 |
| 3 | 96.9 | 113.7 | 60 | 30.8 | 21.6 | 29.2 |
| 4 | 88.9 | 14.3 | 42 | 22.1 | 61.9 | 11.3 |
| 5 | 106 | 20.6 | 43.6 | 20.4 | 19.7 | 30 |
| 6 | 117.6 | 31.6 | 20.5 | 30.6 | 14.2 | 33.6 |
| 7 | 70.6 | 12.6 | 13 | 21.1 | N.D. | 11.9 |
| Mean | 100.00 | 31.10 | 31.94 | 25.96 | 24.93 | 20.43 |

Table 27. shows % mouse APP mRNA remaining relative to PBS control in the striatum 14 days post dosing. N=7. N.D.: Not determined.

| animal | % remaining of mAPP mRNA | | | | | |
|---|---|---|---|---|---|---|
| No. | PBS | DSR-0103199 | DSR-0103216 | DSR-0103299 | DSR-0103300 | DSR-0103301 |
| 1 | 86.90 | 56.60 | 52.10 | 15.90 | 45.70 | 33.50 |
| 2 | 125.10 | 14.00 | 17.40 | 37.30 | 53.40 | 13.00 |
| 3 | 90.00 | 18.10 | 26.30 | 22.10 | 28.80 | 11.40 |
| 4 | 96.20 | 90.60 | 19.30 | 19.70 | 40.00 | 13.60 |
| 5 | N.D. | 40.80 | 77.40 | 16.90 | 37.00 | 16.50 |
| 6 | 99.80 | 21.50 | 31.50 | 27.70 | 30.40 | N.D. |
| 7 | 102.10 | 12.60 | 23.10 | 46.90 | 14.90 | 19.30 |
| Mean | 85.73 | 36.31 | 35.30 | 26.64 | 35.74 | 15.33 |

Table 28. shows % mouse APP mRNA remaining relative to PBS control in the striatum 56 days post dosing. N=7. N.D.: Not determined.

| animal | % remaining of mAPP mRNA | | | | | |
|---|---|---|---|---|---|---|
| No. | PBS | DSR-0103199 | DSR-0103216 | DSR-0103299 | DSR-0103300 | DSR-0103301 |
| 1 | 104.40 | 8.40 | 16.00 | 73.60 | 28.60 | 8.10 |
| 2 | 102.00 | 8.80 | N.D. | 14.70 | 14.60 | 12.90 |
| 3 | 104.50 | 76.30 | 59.10 | 25.10 | 32.40 | 11.10 |
| 4 | 99.70 | 13.10 | 20.10 | 17.40 | 18.00 | 13.00 |
| 5 | 88.10 | 19.40 | 39.50 | 17.80 | 17.30 | 36.40 |
| 6 | 100.70 | 14.30 | 20.10 | 29.70 | 11.80 | 26.20 |
| 7 | 100.60 | 10.70 | 22.30 | 19.50 | N.D. | 21.80 |
| Mean | 100.00 | 21.57 | 25.30 | 28.26 | 17.53 | 18.50 |

Table 29. shows % mouse APP mRNA remaining relative to PBS control in the cerebellum 14 days post dosing. N=7. N.D.: Not determined.

| animal | % remaining of mAPP mRNA | | | | | |
|---|---|---|---|---|---|---|
| No. | PBS | DSR-0103199 | DSR-0103216 | DSR-0103299 | DSR-0103300 | DSR-0103301 |
| 1 | 99.30 | 52.10 | 44.70 | 32.40 | 39.80 | 51.30 |
| 2 | 92.40 | 28.60 | 33.00 | 44.50 | 39.50 | 18.80 |
| 3 | 102.70 | 32.90 | 27.20 | 41.20 | 27.20 | 34.90 |
| 4 | 92.20 | 64.10 | 45.60 | 45.60 | 42.50 | 16.80 |
| 5 | 104.00 | 58.50 | 65.40 | 37.80 | 69.50 | 18.80 |
| 6 | 108.00 | 38.60 | 33.00 | 41.60 | 31.90 | 19.20 |
| 7 | 101.40 | 12.50 | 31.70 | 43.90 | 24.90 | 22.50 |
| Mean | 100.00 | 41.04 | 40.09 | 41.00 | 39.33 | 26.04 |

Table 29. shows % mouse APP mRNA remaining relative to PBS control in the cerebellum 56 days post dosing. N=7. N.D.: Not determined.

| animal | % remaining of mAPP mRNA | | | | | |
|---|---|---|---|---|---|---|
| No. | PBS | DSR-0103199 | DSR-0103216 | DSR-0103299 | DSR-0103300 | DSR-0103301 |
| 1 | 105.30 | 20.10 | 28.60 | 80.80 | 20.90 | 16.60 |
| 2 | 86.00 | 20.20 | 24.20 | 19.40 | 22.10 | 14.10 |
| 3 | 90.80 | 78.70 | 52.80 | 25.90 | 23.70 | 20.30 |
| 4 | 81.60 | 20.90 | 35.90 | 20.30 | 27.40 | 18.30 |
| 5 | 103.10 | 25.30 | 42.60 | 27.40 | 23.40 | 22.90 |
| 6 | 109.80 | 11.10 | 29.50 | 30.90 | 21.60 | 26.10 |
| 7 | 123.40 | 23.20 | 32.60 | 42.10 | N.D. | 20.20 |
| Mean | 100.00 | 28.50 | 35.17 | 35.26 | 19.87 | 19.79 |

Table 30. shows % mouse APP mRNA remaining relative to PBS control in the hippocampus 14 days post dosing. N=7. N.D.: Not determined.

| animal | % remaining of mAPP mRNA | | | | | |
|---|---|---|---|---|---|---|
| No. | PBS | DSR-0103199 | DSR-0103216 | DSR-0103299 | DSR-0103300 | DSR-0103301 |
| 1 | 85.80 | 20.60 | 16.70 | 19.70 | 21.90 | 27.70 |
| 2 | 107.40 | 7.20 | 17.70 | 23.50 | 16.30 | 8.30 |
| 3 | 85.60 | 8.90 | 8.30 | 19.50 | 16.60 | 21.70 |
| 4 | 93.90 | 50.40 | 7.20 | 21.60 | 15.20 | 11.80 |
| 5 | 86.40 | 22.30 | 57.20 | 55.10 | 27.90 | 13.10 |
| 6 | 104.10 | 18.10 | 12.20 | 18.80 | 13.00 | 8.10 |
| 7 | 136.80 | 9.40 | 11.20 | 21.60 | 12.00 | 14.80 |
| Mean | 100.00 | 19.56 | 18.64 | 25.69 | 17.56 | 15.07 |

Table 31. shows % mouse APP mRNA remaining relative to PBS control in the hippocampus 56 days post dosing. N=7. N.D.: Not determined.

| animal | % remaining of mAPP mRNA | | | | | |
|---|---|---|---|---|---|---|
| No. | PBS | DSR-0103199 | DSR-0103216 | DSR-0103299 | DSR-0103300 | DSR-0103301 |
| 1 | 102.70 | 24.40 | 7.20 | 42.10 | 14.20 | 6.90 |
| 2 | 107.50 | 7.20 | 57.10 | N.D. | 11.80 | 7.60 |
| 3 | 101.60 | 45.10 | 32.10 | 14.50 | 13.70 | 13.60 |
| 4 | 113.70 | 8.20 | 13.60 | 8.40 | 20.20 | 7.50 |
| 5 | 108.90 | 6.60 | 28.50 | 9.80 | 13.40 | 13.20 |
| 6 | 89.20 | 27.10 | 7.50 | 16.00 | 7.20 | 8.50 |
| 7 | 76.30 | 6.00 | 10.50 | 13.80 | N.D. | 7.30 |
| Mean | 100.00 | 17.80 | 22.36 | 14.94 | 11.50 | 9.23 |

Table 32. shows % mouse APP mRNA remaining relative to PBS control in the brainstem 14 days post dosing. N=7. N.D.: Not determined.

| animal | % remaining of mAPP mRNA | | | | | |
|---|---|---|---|---|---|---|
| No. | PBS | DSR-0103199 | DSR-0103216 | DSR-0103299 | DSR-0103300 | DSR-0103301 |
| 1 | 99.40 | 27.00 | 25.20 | 14.80 | 22.80 | 41.40 |
| 2 | 93.60 | 10.20 | 14.70 | 28.90 | 24.90 | 13.20 |
| 3 | 95.80 | 13.50 | 12.50 | 30.00 | 18.50 | 20.50 |
| 4 | 91.20 | 54.20 | 15.10 | 26.00 | 23.60 | 11.60 |
| 5 | 117.30 | 34.80 | 49.80 | 36.90 | 38.50 | 13.90 |
| 6 | 99.00 | 20.40 | 17.30 | 25.90 | 19.70 | 12.50 |
| 7 | 103.60 | 11.30 | 15.10 | 27.00 | 19.00 | 14.00 |
| Mean | 100.00 | 24.49 | 21.39 | 27.07 | 23.86 | 18.16 |

Table 33. shows % mouse APP mRNA remaining relative to PBS control in the brainstem 56 days post dosing. N=7. N.D.: Not determined.

| animal | % remaining of mAPP mRNA | | | | | |
|---|---|---|---|---|---|---|
| No. | PBS | DSR-0103199 | DSR-0103216 | DSR-0103299 | DSR-0103300 | DSR-0103301 |
| 1 | 108.50 | 17.00 | 22.60 | 85.20 | 18.20 | 11.30 |
| 2 | 93.90 | 10.10 | N.D. | 20.50 | 20.20 | 13.20 |
| 3 | 97.70 | 71.10 | 47.70 | 20.60 | 19.80 | 14.50 |
| 4 | 105.00 | 15.20 | 22.20 | 15.00 | 21.20 | 12.40 |
| 5 | 93.00 | 11.10 | 27.80 | 16.30 | 14.60 | 17.90 |
| 6 | 110.00 | 19.60 | 15.90 | 20.90 | 13.90 | 15.40 |
| 7 | 92.00 | 13.60 | 22.20 | 24.00 | N.D. | 13.70 |
| Mean | 100.00 | 22.53 | 22.63 | 28.93 | 15.41 | 14.06 |

Table 34. shows % mouse APP mRNA remaining relative to PBS control in the spinal cord 56 days post dosing. N=7. N.D.: Not determined.

| animal | % remaining of mAPP mRNA | | | | | |
|---|---|---|---|---|---|---|
| No. | PBS | DSR-0103199 | DSR-0103216 | DSR-0103299 | DSR-0103300 | DSR-0103301 |
| 1 | 94.90 | 10.50 | 10.60 | 72.20 | 9.20 | 8.40 |
| 2 | 99.70 | 8.90 | 24.70 | 17.30 | 11.70 | 8.00 |
| 3 | 99.20 | 71.40 | 33.30 | 16.50 | 11.70 | 9.70 |
| 4 | 108.10 | 19.50 | 14.60 | 10.80 | 17.40 | 9.70 |
| 5 | 97.80 | 11.10 | 14.80 | 16.70 | 10.90 | 13.50 |
| 6 | 104.50 | 11.50 | 11.50 | 18.00 | 10.90 | 13.10 |
| 7 | 95.70 | 19.80 | 20.20 | 24.60 | N.D. | 8.80 |
| Mean | 100.00 | 21.81 | 18.53 | 25.16 | 10.26 | 10.17 |

Table 35. shows changes in mouse GFAP mRNA remaining relative to PBS control in the cerebellum 14 days post dosing. N=7. N.D.: Not determined.

| animal | Relative change of mGFAP mRNA | | | | | |
|---|---|---|---|---|---|---|
| No. | PBS | DSR-0103199 | DSR-0103216 | DSR-0103299 | DSR-0103300 | DSR-0103301 |
| 1 | 0.93 | 1.23 | 0.96 | 0.74 | 1.02 | 0.86 |
| 2 | 0.92 | 1.20 | 1.18 | 0.90 | 1.06 | 1.06 |
| 3 | 1.11 | 1.45 | 1.68 | 0.81 | 0.87 | 0.88 |
| 4 | 0.95 | 0.81 | 1.11 | 1.07 | 1.03 | 1.21 |
| 5 | 0.88 | 1.31 | 0.99 | 1.09 | 1.32 | 1.12 |
| 6 | 1.17 | 1.60 | 1.37 | 1.11 | 1.15 | 1.40 |
| 7 | 1.04 | 1.14 | 1.79 | 1.10 | 1.03 | 1.00 |
| Mean | 1.00 | 1.25 | 1.30 | 0.97 | 1.07 | 1.08 |

Table 36. shows changes in mouse GFAP mRNA remaining relative to PBS control in the cerebellum 56 days post dosing. N=7. N.D.: Not determined.

| animal | Relative change of mGFAP mRNA | | | | | |
|---|---|---|---|---|---|---|
| No. | PBS | DSR-0103199 | DSR-0103216 | DSR-0103299 | DSR-0103300 | DSR-0103301 |
| 1 | 1.09 | 0.64 | 0.91 | 1.00 | 1.07 | 0.97 |
| 2 | 0.86 | 1.09 | N.D. | 0.80 | 6.32 | 0.98 |
| 3 | 1.10 | 1.08 | 0.84 | 1.13 | 0.81 | 1.08 |
| 4 | 0.78 | 0.88 | 0.86 | 1.05 | 1.03 | 0.83 |
| 5 | 1.10 | 1.23 | 0.97 | 1.08 | 0.89 | 1.22 |
| 6 | 0.96 | 1.86 | 0.89 | 1.13 | 0.96 | 1.22 |
| 7 | 1.10 | 1.01 | 1.07 | 1.12 | N.D. | 1.01 |
| Mean | 1.00 | 1.11 | 0.79 | 1.04 | 1.58 | 1.04 |

Table 37. shows changes in mouse GFAP mRNA remaining relative to PBS control in the brainstem 14 days post dosing. N=7. N.D.: Not determined.

| | Relative change of mGFAP mRNA | | | | | |
|---|---|---|---|---|---|---|
| animal No. | PBS | DSR-0103199 | DSR-0103216 | DSR-0103299 | DSR-0103300 | DSR-0103301 |
| 1 | 1.07 | 1.22 | 1.52 | 1.06 | 0.91 | 0.87 |
| 2 | 1.03 | 1.43 | 1.54 | 0.92 | 1.00 | 0.90 |
| 3 | 1.02 | 1.34 | 1.67 | 0.95 | 0.60 | 0.55 |
| 4 | 0.86 | 0.76 | 1.59 | 1.34 | 0.82 | 0.84 |
| 5 | 1.25 | 1.17 | 1.39 | 0.84 | 1.51 | 0.77 |
| 6 | 0.85 | 0.99 | 1.89 | 0.90 | 0.84 | 1.02 |
| 7 | 0.92 | 1.53 | 1.92 | 0.89 | 1.03 | 0.60 |
| Mean | 1.00 | 1.21 | 1.65 | 0.99 | 0.96 | 0.79 |

Table 38. shows changes in mouse GFAP mRNA remaining relative to PBS control in the brainstem 56 days post dosing. N=7. N.D.: Not determined.

| | Relative change of mGFAP mRNA | | | | | |
|---|---|---|---|---|---|---|
| animal No. | PBS | DSR-0103199 | DSR-0103216 | DSR-0103299 | DSR-0103300 | DSR-0103301 |
| 1 | 1.06 | 0.80 | 0.66 | 0.93 | 0.84 | 1.15 |
| 2 | 1.30 | 0.89 | N.D. | 0.94 | 4.45 | 0.75 |
| 3 | 0.56 | 0.86 | 0.74 | 0.74 | 0.90 | 0.83 |
| 4 | 0.85 | 0.99 | 0.93 | 0.88 | 0.68 | 1.10 |
| 5 | 1.19 | 0.95 | 0.71 | 1.02 | 0.65 | 0.73 |
| 6 | 0.98 | 0.90 | 0.67 | 0.84 | 1.04 | 0.87 |
| 7 | 1.05 | 1.35 | 0.76 | 0.78 | N.D. | 0.97 |
| Mean | 1.00 | 0.96 | 0.64 | 0.88 | 1.22 | 0.91 |

Table 39. shows changes in mouse GFAP mRNA remaining relative to PBS control in the spinal cord 56 days post dosing. N=7. N.D.: Not determined.

| | Relative change of mGFAP mRNA | | | | | |
|---|---|---|---|---|---|---|
| animal No. | PBS | DSR-0103199 | DSR-0103216 | DSR-0103299 | DSR-0103300 | DSR-0103301 |
| 1 | 1.04 | 1.03 | 1.06 | 1.20 | 1.08 | 1.36 |
| 2 | 0.97 | 1.06 | 1.12 | 1.27 | 2.00 | 1.12 |
| 3 | 1.05 | 1.08 | 0.98 | 1.03 | 0.96 | 1.07 |
| 4 | 0.87 | 1.04 | 0.95 | 1.10 | 1.06 | 1.11 |
| 5 | 1.00 | 0.98 | 0.76 | 1.19 | 1.17 | 0.93 |
| 6 | 1.04 | 1.10 | 0.87 | 1.13 | 1.10 | 1.24 |
| 7 | 1.03 | 1.14 | 0.89 | 1.16 | 0.00 | 1.21 |
| Mean | 1.00 | 1.06 | 0.95 | 1.15 | 1.05 | 1.15 |

Table 40. shows changes in mouse AIF1 mRNA remaining relative to PBS control in the cerebellum 14 days post dosing. N=7. N.D.: Not determined.

| | Relative change of mAIF1 mRNA | | | | | |
|---|---|---|---|---|---|---|
| animal No. | PBS | DSR-0103199 | DSR-0103216 | DSR-0103299 | DSR-0103300 | DSR-0103301 |
| 1 | 0.88 | 1.26 | 2.25 | 0.97 | 0.99 | 0.91 |
| 2 | 0.95 | 2.15 | 2.43 | 0.95 | 0.95 | 1.20 |
| 3 | 0.99 | 3.04 | 3.82 | 0.99 | 0.72 | 0.98 |
| 4 | 1.00 | 1.03 | 2.14 | 1.22 | 1.10 | 1.49 |
| 5 | 0.80 | 1.69 | 1.37 | 0.94 | 2.74 | 0.95 |
| 6 | 0.94 | 3.24 | 2.81 | 0.89 | 0.91 | 1.81 |
| 7 | 1.44 | 1.69 | 2.83 | 0.82 | 1.06 | 1.74 |
| Mean | 1.00 | 2.01 | 2.52 | 0.97 | 1.21 | 1.30 |

Table 41. shows changes in mouse AIF1 mRNA remaining relative to PBS control in the cerebellum 56 days post dosing. N=7. N.D.: Not determined.

| | Relative change of mAIF1 mRNA | | | | | |
|---|---|---|---|---|---|---|
| animal No. | PBS | DSR-0103199 | DSR-0103216 | DSR-0103299 | DSR-0103300 | DSR-0103301 |
| 1 | 1.01 | 0.99 | 1.43 | 0.93 | 0.99 | 1.06 |
| 2 | 0.88 | 1.46 | 0.00 | 0.73 | 9.58 | 1.01 |
| 3 | 0.82 | 1.03 | 0.93 | 0.95 | 0.87 | 0.89 |
| 4 | 0.89 | 1.43 | 1.16 | 1.05 | 0.96 | 0.73 |
| 5 | 1.13 | 1.42 | 1.00 | 1.00 | 0.91 | 1.10 |
| 6 | 0.96 | 2.23 | 1.17 | 0.95 | 0.95 | 0.79 |
| 7 | 1.30 | 1.46 | 1.37 | 0.94 | 0.00 | 0.91 |
| Mean | 1.00 | 1.43 | 1.01 | 0.94 | 2.04 | 0.93 |

Table 42. shows changes in mouse AIF1 mRNA remaining relative to PBS control in the brainstem 14 days post dosing. N=7. N.D.: Not determined.

| | Relative change of mAIF1 mRNA | | | | | |
|---|---|---|---|---|---|---|
| animal No. | PBS | DSR-0103199 | DSR-0103216 | DSR-0103299 | DSR-0103300 | DSR-0103301 |
| 1 | 1.09 | 1.40 | 1.85 | 1.12 | 1.06 | 1.31 |
| 2 | 0.93 | 1.99 | 2.12 | 0.98 | 1.14 | 1.01 |
| 3 | 0.97 | 2.28 | 2.41 | 1.01 | 1.01 | 1.15 |
| 4 | 0.99 | 1.08 | 1.67 | 1.37 | 0.83 | 0.90 |
| 5 | 0.93 | 1.25 | 1.42 | 0.82 | 2.63 | 0.59 |
| 6 | 0.82 | 1.26 | 2.50 | 0.71 | 0.73 | 1.35 |
| 7 | 1.27 | 1.60 | 2.46 | 1.05 | 1.02 | 0.85 |
| Mean | 1.00 | 1.55 | 2.06 | 1.01 | 1.20 | 1.02 |

Table 43. shows changes in mouse AIF1 mRNA remaining relative to PBS control in the brainstem 56 days post dosing. N=7. N.D.: Not determined.

| | Relative change of mAIF1 mRNA | | | | | |
|---|---|---|---|---|---|---|
| animal No. | PBS | DSR-0103199 | DSR-0103216 | DSR-0103299 | DSR-0103300 | DSR-0103301 |
| 1 | 1.22 | 1.24 | 1.17 | 1.02 | 0.87 | 0.87 |
| 2 | 0.96 | 1.44 | 0.00 | 0.80 | 5.80 | 0.68 |
| 3 | 0.84 | 0.79 | 0.80 | 0.80 | 0.87 | 0.78 |
| 4 | 0.91 | 1.17 | 1.01 | 0.76 | 1.07 | 0.85 |
| 5 | 1.24 | 1.27 | 0.99 | 0.84 | 0.74 | 0.74 |
| 6 | 0.66 | 1.29 | 0.75 | 0.74 | 0.85 | 0.77 |
| 7 | 1.17 | 1.43 | 0.83 | 0.73 | 0.00 | 0.81 |
| Mean | 1.00 | 1.23 | 0.79 | 0.81 | 1.46 | 0.79 |

Table 44. shows changes in mouse AIF1 mRNA remaining relative to PBS control in the spinal cord 56 days post dosing. N=7. N.D.: Not determined.

| | Relative change of mAIF1 mRNA | | | | | |
|---|---|---|---|---|---|---|
| animal No. | PBS | DSR-0103199 | DSR-0103216 | DSR-0103299 | DSR-0103300 | DSR-0103301 |
| 1 | 1.08 | 1.21 | 1.63 | 1.03 | 0.95 | 1.08 |
| 2 | 0.96 | 1.60 | 1.28 | 1.21 | 4.91 | 1.15 |
| 3 | 0.88 | 0.97 | 1.28 | 0.91 | 1.06 | 1.08 |
| 4 | 0.89 | 1.35 | 1.31 | 1.12 | 1.11 | 1.05 |
| 5 | 1.12 | 1.23 | 1.08 | 1.12 | 0.98 | 1.09 |
| 6 | 1.03 | 1.41 | 1.27 | 1.14 | 0.86 | 0.99 |
| 7 | 1.03 | 1.29 | 1.37 | 1.01 | N.D. | 1.13 |
| Mean | 1.00 | 1.30 | 1.32 | 1.08 | 1.41 | 1.08 |

Table 45. shows changes in mouse PCP2 mRNA remaining relative to PBS control in the cerebellum-14 days-pos-t-dosing. N=7. N.D.: Not determined.

| | Relative change of mPCP2 mRNA | | | | | |
|---|---|---|---|---|---|---|
| animal No. | PBS | DSR-0103199 | DSR-0103216 | DSR-0103299 | DSR-0103300 | DSR-0103301 |
| 1 | 0.96 | 0.97 | 1.05 | 0.98 | 1.04 | 1.26 |
| 2 | 1.01 | 1.07 | 1.00 | 1.28 | 1.50 | 0.85 |
| 3 | 1.11 | 1.15 | 0.64 | 0.95 | 0.92 | 1.06 |
| 4 | 1.23 | 0.89 | 1.19 | 1.06 | 1.08 | 1.00 |
| 5 | 0.67 | 1.20 | 0.92 | 0.80 | 0.79 | 0.96 |
| 6 | 1.14 | 1.22 | 1.08 | 1.01 | 0.87 | 1.06 |
| 7 | 0.87 | 0.00 | 1.31 | 1.00 | 1.12 | 1.42 |
| Mean | 1.00 | 0.93 | 1.03 | 1.01 | 1.05 | 1.09 |

Table 46. shows changes in mouse PCP2 mRNA remaining relative to PBS control in the cerebellum 56 days post dosing. N=7. N.D.: Not determined.

| | Relative change of mPCP2 mRNA | | | | | |
|---|---|---|---|---|---|---|
| animal No. | PBS | DSR-0103199 | DSR-0103216 | DSR-0103299 | DSR-0103300 | DSR-0103301 |
| 1 | 0.82 | 0.56 | 0.59 | 0.55 | 0.61 | 0.63 |
| 2 | 0.52 | 0.75 | 0.43 | 0.39 | 0.61 | 0.69 |
| 3 | 0.74 | 0.73 | 0.68 | 0.60 | 0.65 | 0.70 |
| 4 | 0.81 | 0.87 | 0.64 | 0.62 | 0.54 | 0.78 |
| 5 | 1.78 | 0.97 | 0.40 | 0.68 | 0.65 | 0.85 |
| 6 | 1.71 | 0.00 | 0.77 | 0.77 | 0.84 | 0.93 |
| 7 | 0.62 | 0.87 | 0.70 | 0.81 | N.D. | 1.04 |
| Mean | 1.00 | 0.68 | 0.60 | 0.63 | 0.56 | 0.80 |

Table 47. shows changes in mouse GRID2 mRNA remaining relative to PBS control in the cerebellum 14 days post dosing. N=7. N.D.: Not determined.

| | Relative change of mGRID2 mRNA | | | | | |
|---|---|---|---|---|---|---|
| animal No. | PBS | DSR-0103199 | DSR-0103216 | DSR-0103299 | DSR-0103300 | DSR-0103301 |
| 1 | 0.99 | 1.08 | 1.12 | 1.11 | 1.25 | 1.27 |
| 2 | 0.87 | 1.18 | 1.01 | 1.19 | 1.08 | 0.95 |
| 3 | 1.12 | 1.14 | 0.72 | 0.96 | 1.09 | 1.06 |
| 4 | 1.11 | 0.97 | 0.92 | 1.03 | 1.01 | 1.02 |
| 5 | 0.78 | 1.12 | 1.00 | 0.82 | 0.93 | 1.07 |
| 6 | 1.00 | 1.12 | 1.01 | 1.09 | 0.95 | 1.10 |
| 7 | 1.13 | 0.04 | 1.06 | 1.16 | 1.28 | 1.70 |
| Mean | 1.00 | 0.95 | 0.98 | 1.05 | 1.09 | 1.17 |

Table 48. shows changes in mouse GRID2 mRNA remaining relative to PBS control in the cerebellum 56 days post dosing. N=7. N.D.: Not determined.

| | Relative change of mGRID2 mRNA | | | | | |
|---|---|---|---|---|---|---|
| animal No. | PBS | DSR-0103199 | DSR-0103216 | DSR-0103299 | DSR-0103300 | DSR-0103301 |
| 1 | 0.87 | 0.79 | 1.04 | 1.02 | 1.07 | 1.23 |
| 2 | 0.82 | 1.28 | 0.57 | 0.56 | 0.98 | 1.17 |
| 3 | 0.98 | 1.16 | 1.17 | 0.87 | 1.21 | 1.20 |
| 4 | 0.81 | 1.09 | 1.19 | 0.97 | 0.88 | 1.21 |
| 5 | 1.03 | 1.21 | 0.60 | 0.99 | 0.83 | 1.26 |
| 6 | 0.99 | 0.06 | 1.21 | 1.11 | 1.08 | 1.27 |
| 7 | 1.50 | 1.61 | 1.49 | 1.40 | N.D. | 1.70 |
| Mean | 1.00 | 1.03 | 1.04 | 0.99 | 0.86 | 1.29 |

Table 49. shows changes in mouse CALBINDIN1 mRNA remaining relative to PBS control in the cerebellum 14 days post dosing. N=7. N.D.: Not determined.

| | Relative change of m CALBINDIN1 mRNA | | | | | |
|---|---|---|---|---|---|---|
| animal No. | PBS | DSR-0103199 | DSR-0103216 | DSR-0103299 | DSR-0103300 | DSR-0103301 |
| 1 | 0.95 | 1.04 | 1.04 | 0.75 | 1.09 | 1.06 |
| 2 | 0.96 | 0.91 | 1.18 | 1.08 | 1.13 | 0.96 |
| 3 | 1.32 | 0.99 | 0.81 | 0.98 | 0.80 | 0.67 |
| 4 | 1.16 | 1.02 | 0.72 | 0.97 | 1.08 | 1.09 |
| 5 | 0.76 | 1.00 | 0.77 | 0.77 | 0.56 | 1.07 |
| 6 | 0.88 | 0.98 | 0.89 | 0.85 | 0.97 | 1.06 |
| 7 | 0.97 | 0.03 | 1.00 | 0.86 | 1.11 | 0.89 |
| Mean | 1.00 | 0.85 | 0.92 | 0.89 | 0.96 | 0.97 |

Table 50. shows changes in mouse CALBINDIN1 mRNA remaining relative to PBS control in the cerebellum 56 days post dosing. N=7. N.D.: Not determined.

| | Relative change of m CALBINDIN1 mRNA | | | | | |
|---|---|---|---|---|---|---|
| animal No. | PBS | DSR-0103199 | DSR-0103216 | DSR-0103299 | DSR-0103300 | DSR-0103301 |
| 1 | 1.14 | 0.99 | 1.05 | 1.13 | 1.09 | 1.09 |
| 2 | 0.83 | 1.08 | 0.62 | 0.68 | 0.82 | 1.10 |
| 3 | 0.95 | 1.29 | 0.92 | 0.77 | 1.05 | 1.05 |
| 4 | 1.07 | 1.09 | 0.95 | 1.02 | 0.86 | 0.90 |
| 5 | 0.95 | 1.03 | 0.48 | 0.89 | 0.78 | 1.13 |
| 6 | 1.16 | 0.12 | 0.94 | 1.11 | 0.98 | 1.07 |
| 7 | 0.91 | 0.92 | 0.94 | 1.01 | 0.00 | 1.09 |
| Mean | 1.00 | 0.93 | 0.84 | 0.94 | 0.80 | 1.06 |

Table 51. shows the accumulation of antisense strand in the cortex 14 days post dosing. N=7. N.D.: Not determined.

| | Cortex Guide Strand PK (ug oligo/g tissue) | | | | | |
|---|---|---|---|---|---|---|
| Aimal No. | PBS | DSR-0103199 | DSR-0103216 | DSR-0103299 | DSR-0103300 | DSR-0103301 |
| 1 | 0 | 1.33948 | 1.80608 | 3.82596 | 2.8835 | 0.66222 |
| 2 | 0 | 16.8195 | 3.84503 | 1.95844 | 1.86272 | 2.61393 |
| 3 | 0 | 2.85246 | 1.87771 | 15.1319 | 3.79604 | 2.71381 |
| 4 | 0 | 0.50572 | 5.66784 | 18.1493 | 1.77258 | 2.95025 |
| 5 | 0 | 1.06663 | 1.45146 | 6.11727 | 2.16316 | 5.23829 |
| 6 | 0 | 2.35304 | 2.9701 | 2.25564 | 4.74025 | 13.5293 |
| 7 | 0 | 3.67902 | 4.78442 | 2.51718 | 4.55966 | 3.24826 |
| Mean | 0 | 4.08798 | 3.20038 | 7.13653 | 3.11113 | 4.42229 |

Table 52. shows the accumulation of antisense strand in the cortex 56 days post dosing. N=7. N.D.: Not determined.

| Aimal | Cortex Guide Strand PK (ug oligo/g tissue) | | | | | |
|---|---|---|---|---|---|---|
| No. | PBS | DSR-0103199 | DSR-0103216 | DSR-0103299 | DSR-0103300 | DSR-0103301 |
| 3 | 0 | 0.20081 | 0.93402 | 2.00189 | 1.86236 | 2.37207 |
| 4 | 0 | 2.53469 | 1.8332 | 3.21747 | 1.84593 | 2.95649 |
| 5 | 0 | 3.0488 | 0.48963 | 2.58878 | 2.49124 | 0.91171 |
| 6 | 0 | 3.11275 | 9.08395 | 1.71651 | 2.2698 | 2.145 |
| 7 | 0 | 26.6596 | 6.58865 | 7.95356 | | 2.94993 |
| Mean | 0 | 7.11133 | 3.78589 | 3.49564 | 2.11733 | 2.26704 |

Table 53. shows the accumulation of antisense strand in the striatum 14 days post dosing. N=7. N.D.: Not determined.

| Aimal | Striatum Guide Strand PK (ug oligo/g tissue) | | | | | |
|---|---|---|---|---|---|---|
| No. | PBS | DSR-0103199 | DSR-0103216 | DSR-0103299 | DSR-0103300 | DSR-0103301 |
| 1 | 0 | 0.551 | 2.21288 | 2.32673 | 2.53787 | 0.73583 |
| 2 | 0 | 4.12606 | 3.36196 | 1.06003 | 1.21851 | 2.98398 |
| 3 | 0 | 2.18424 | 2.73792 | 5.76885 | 2.45364 | 0.9651 |
| 4 | 0 | 0.50011 | 2.12554 | 4.48963 | 1.52145 | 2.2157 |
| 5 | 0 | 0.61944 | 0.5552 | 1.46772 | 1.96907 | 2.73949 |
| 6 | 0 | 1.43174 | 2.79406 | 0.83037 | 1.87262 | 8.63543 |
| 7 | 0 | 4.75519 | 3.1783 | 1.98539 | 2.93697 | 5.0838 |
| Mean | 0 | 2.02397 | 2.42369 | 2.56125 | 2.07288 | 3.33705 |

Table 54. shows the accumulation of antisense strand in the striatum 56 days post dosing. N=7. N.D.: Not determined.

| Aimal | Striatum Guide Strand PK (ug oligo/g tissue) | | | | | |
|---|---|---|---|---|---|---|
| No. | PBS | DSR-0103199 | DSR-0103216 | DSR-0103299 | DSR-0103300 | DSR-0103301 |
| 3 | 0 | 0.42331 | 0.85208 | 0.7444 | 1.12985 | 2.20913 |
| 4 | 0 | 1.07156 | 2.51691 | 3.6308 | 1.91271 | 3.13026 |
| 5 | 0 | 1.79049 | 1.5583 | 0.78541 | 1.8188 | 1.61048 |
| 6 | 0 | 1.55346 | 1.71599 | 0.9843 | 0.86184 | 2.15115 |
| 7 | 0 | 11.8794 | 6.19197 | 5.16557 | | 9.43752 |
| Mean | 0 | 3.34364 | 2.56705 | 2.2621 | 1.4308 | 3.70771 |

Table 55. shows the accumulation of antisense strand in the hippocampus 14 days post dosing. N=7. N.D.: Not determined.

| Aimal | Hippocampus Guide Strand PK (ug oligo/g tissue) | | | | | |
|---|---|---|---|---|---|---|
| No. | PBS | DSR-0103199 | DSR-0103216 | DSR-0103299 | DSR-0103300 | DSR-0103301 |
| 1 | 0 | 2.81788 | 3.90611 | 3.31764 | 2.09342 | 1.03392 |
| 2 | 0 | 15.6481 | 5.6271 | 1.34039 | 1.5961 | 4.11233 |
| 3 | 0 | 5.18201 | 4.0548 | 5.6222 | 2.47653 | 1.63066 |
| 4 | 0 | 0.86528 | 5.96447 | 35.2191 | 1.04451 | 3.02446 |
| 5 | 0 | 1.20395 | 2.13628 | 11.0307 | 1.64763 | 3.54461 |
| 6 | 0 | 2.57232 | 7.11894 | 1.19691 | 3.50326 | 28.5453 |
| 7 | 0 | 5.39943 | 9.24162 | 1.93582 | 5.84708 | 1.62606 |
| Mean | 0 | 4.81271 | 5.43562 | 8.52325 | 2.60122 | 6.21676 |

Table 56. shows the accumulation of antisense strand in the hippocampus 56 days post dosing. N=7. N.D.: Not determined.

| Aimal | Hippocampus Guide Strand PK (ug oligo/g tissue) | | | | | |
|---|---|---|---|---|---|---|
| No. | PBS | DSR-0103199 | DSR-0103216 | DSR-0103299 | DSR-0103300 | DSR-0103301 |
| 3 | 0 | 0.22335 | 1.51477 | 2.20613 | 2.3042 | 2.98112 |
| 4 | 0 | 6.3419 | 2.4054 | 2.34931 | 2.30888 | 4.40675 |
| 5 | 0 | 3.83749 | 1.90508 | 2.74854 | 5.23838 | 1.74007 |
| 6 | 0 | 2.17971 | 9.00748 | 1.71049 | 2.99417 | 3.75598 |
| 7 | 0 | 23.3133 | 14.5841 | 9.43574 | | 5.2144 |
| Mean | 0 | 7.17915 | 5.88337 | 3.69004 | 3.21141 | 3.61966 |

Table 57. shows the accumulation of antisense strand in the cerebellum 14 days post dosing. N=7. N.D.: Not determined.

| Aimal | Cerebellum Guide Strand PK (ug oligo/g tissue) | | | | | |
|---|---|---|---|---|---|---|
| No. | PBS | DSR-0103199 | DSR-0103216 | DSR-0103299 | DSR-0103300 | DSR-0103301 |
| 1 | 0 | 1.40272 | 2.7092 | 3.72023 | 2.54973 | 0.79629 |
| 2 | 0 | 5.82978 | 5.76795 | 1.89276 | 1.64845 | 4.96995 |
| 3 | 0 | 3.79642 | 6.6563 | 2.65203 | 3.61692 | 2.17467 |
| 4 | 0 | 0.59886 | 7.38686 | 5.00374 | 2.06531 | 5.45346 |
| 5 | 0 | 1.44633 | 0.7256 | 2.3963 | 1.0604 | 4.8901 |
| 6 | 0 | 3.1469 | 4.99195 | 2.31226 | 3.37586 | 6.84859 |
| 7 | 0 | 6.31042 | 6.15305 | 2.08616 | 4.4272 | 4.68073 |
| Mean | 0 | 3.21878 | 4.91299 | 2.86621 | 2.6777 | 4.25911 |

Table 58. shows the accumulation of antisense strand in the cerebellum 56 days post dosing. N=7. N.D.: Not determined.

| Aimal | Cerebellum Guide Strand PK (ug oligo/g tissue) | | | | | |
|---|---|---|---|---|---|---|
| No. | PBS | DSR-0103199 | DSR-0103216 | DSR-0103299 | DSR-0103300 | DSR-0103301 |
| 3 | 0 | 0.15425 | 1.06097 | 2.0099 | 2.56686 | 4.3132 |
| 4 | 0 | 3.11745 | 2.08099 | 3.05043 | 1.0629 | 4.87016 |
| 5 | 0 | 5.12822 | 1.92624 | 2.1483 | 4.51151 | 2.93411 |
| 6 | 0 | 1.90138 | 4.63673 | 1.91964 | 3.78654 | 2.31057 |
| 7 | 0 | 4.8623 | 4.15376 | 2.11323 | | 4.6793 |
| Mean | 0 | 3.03272 | 2.77174 | 2.2483 | 2.98195 | 3.82147 |

Table 59. shows the accumulation of antisense strand in the brain stem14 days post dosing. N=7. N.D.: Not determined.

| Aimal | Brain Stem Guide Strand PK (ug oligo/g tissue) | | | | | |
|---|---|---|---|---|---|---|
| No. | PBS | DSR-0103199 | DSR-0103216 | DSR-0103299 | DSR-0103300 | DSR-0103301 |
| 1 | 0 | 2.77113 | 4.0305 | 3.56911 | 2.96699 | 1.08657 |
| 2 | 0 | 8.36009 | 4.857 | 2.03299 | 2.25413 | 5.09715 |
| 3 | 0 | 5.36779 | 7.76106 | 3.75237 | 4.66602 | 2.98417 |
| 4 | 0 | 1.35822 | 11.5525 | 16.0687 | 2.57904 | 6.48715 |
| 5 | 0 | 2.40431 | 1.7073 | 5.46635 | 1.66483 | 3.52442 |
| 6 | 0 | 3.31405 | 6.95348 | 2.31155 | 4.1131 | 12.8219 |
| 7 | 0 | 7.87811 | 11.0572 | 2.50131 | 5.24387 | 4.77719 |
| Mean | 0 | 4.49339 | 6.84558 | 5.10034 | 3.35543 | 5.25408 |

Table 60. shows the accumulation of antisense strand in the brain stem 56 days post dosing. N=7. N.D.: Not determined.

| Aimal | Brain Stem Guide Strand PK (ug oligo/g tissue) | | | | | |
|---|---|---|---|---|---|---|
| No. | PBS | DSR-0103199 | DSR-0103216 | DSR-0103299 | DSR-0103300 | DSR-0103301 |
| 3 | 0 | 0.40218 | 1.55602 | 2.05998 | 2.26861 | 3.13505 |
| 4 | 0 | 7.497 | 2.57235 | 3.62769 | 2.61901 | 3.91605 |
| 5 | 0 | 5.24135 | 1.89323 | 2.50807 | 4.59302 | 1.8709 |
| 6 | 0 | 2.5117 | 4.55858 | 2.05992 | 4.99378 | 2.50165 |
| 7 | 0 | 15.1391 | 9.30655 | 5.36508 | | 4.79568 |
| Mean | 0 | 6.15827 | 3.97735 | 3.12415 | 3.61861 | 3.24387 |

Table 61. shows the accumulation of antisense strand in the spinal cord 56 days post dosing. N=7. N.D.: Not determined.

| Aimal | Spinal Cord Guide Strand PK (ug oligo/g tissue) | | | | | |
|---|---|---|---|---|---|---|
| No. | PBS | DSR-0103199 | DSR-0103216 | DSR-0103299 | DSR-0103300 | DSR-0103301 |
| 1 | 0 | 0.33782 | 0.38522 | 0.0687 | 0.38175 | 0.8507 |
| 2 | 0 | 0.72056 | 0.17588 | 0.22064 | 0.32037 | 0.70698 |
| 3 | 0 | 0.08108 | 0.16001 | 0.20748 | 0.29364 | 0.46593 |
| 4 | 0 | 0.21632 | 0.31657 | 0.33833 | 0.21955 | 0.64514 |
| 5 | 0 | 0.4908 | 0.29841 | 0.20039 | 0.5234 | 0.38035 |
| 6 | 0 | 0.44083 | 0.4376 | 0.18571 | 0.38424 | 0.3374 |
| 7 | 0 | 0.22957 | 0.22118 | 0.16681 | | 0.58253 |
| Mean | 0 | 0.35957 | 0.28498 | 0.19829 | 0.35383 | 0.567 |

Table 61a. shows Ago 2 loading of guide strand relative to miR-124 in cortex. N=3.

| | Day 56 | | | | |
|---|---|---|---|---|---|
| | Ct: | Ct: | Ct: | Ct: | Relative |
| | mAPP/ | miR- | mAPP/ | miR- | mAPP/miR- |
| | Ago2 | 124/Ago2 | IgG | 124/IgG | 124 |
| PBS-1 | 33.48 | 30.09 | 32.62 | 38.6 | 0.08 |
| PBS-2 | 33.28 | 28.83 | 33.51 | 39.01 | 0.01 |
| PBS-3 | 32.27 | 28.16 | 32.65 | 38.56 | 0.01 |
| DSR-0103199-1 | 22.89 | 28.72 | 28.53 | 40 | 55.75 |
| DSR-0103199-2 | 21.32 | 28.78 | 27.2 | 37.89 | 173.08 |
| DSR-0103199-3 | 27.35 | 27.51 | 31.03 | 37.53 | 1.03 |
| DSR-0103216-1 | 20.59 | 28.03 | 26.94 | 36.64 | 171.52 |
| DSR-0103216-2 | 25.78 | 31.48 | 29.46 | 40 | 47.93 |
| DSR-0103216-3 | 24.65 | 28.89 | 28.74 | 36 | 17.79 |
| DSR-0103299-1 | 24.31 | 30.76 | 29.03 | 35.82 | 84.11 |
| DSR-0103299-2 | 22.55 | 28.33 | 26.41 | 36.1 | 51.16 |
| DSR-0103299-3 | 24.21 | 30.34 | 29.03 | 36.42 | 67.56 |
| DSR-0103300-1 | 23.63 | 29.16 | 28.72 | 37.79 | 44.85 |
| DSR-0103300-2 | 22.08 | 29.23 | 27.49 | 37.56 | 138.68 |
| DSR-0103300-3 | 23.52 | 28.31 | 28.77 | 36.2 | 26.94 |
| DSR-0103301-1 | 20.27 | 27.47 | 26.48 | 33.72 | 145.05 |
| DSR-0103301-2 | 22.80 | 28.74 | 27.42 | 35.09 | 58.90 |
| DSR-0103301-3 | 22.19 | 27.3 | 26.65 | 33.36 | 32.97 |

Table 61b. shows average serum NfL levels 4, 6, or 8 weeks post siRNA administration. N=7. N.D.: Not determined.

| | Group average NfL (pg/ml) | | |
|---|---|---|---|
| Group | Week 2 | Week 4 | Week 8 |
| PBS | 1383 | 1413 | 1440 |
| DSR-0103199 | 519.6 | 1014 | 826.5 |
| DSR-0103216 | 826.2 | 1017 | 1281 |
| DSR-0103299 | 1946 | 2563 | 1641 |
| DSR-0103300 | 744.5 | 981.3 | 1464 |
| DSR-0103301 | 746.9 | 860.7 | 724.5 |

Table 61c. shows gene numbers for downregulated, unchanged, and upregulated genes in RNAseq.

| | Downregulated | Unchanged | Upregulated |
|---|---|---|---|
| DSR-0103199 | 9 | 55156 | 256 |
| DSR-0103299 | 1 | 55420 | 0 |

### Provided Oligonucleotides and Compositions are Active In Situ

Various siRNAs for APP siRNA distribution in brain tissue were designed and constructed. A number of siRNAs were tested in situ using a ViewRNA in situ hybridization (ISH) Assay. Example protocol for ViewRNA in situ hybridization assay: Mouse brain hemisphere were fixed in 10% neutral buffered formalin overnight at 2-8° C., processed and embedded in paraffin. The tissue was cut into 5 μm sections and evaluated by using ViewRNA ISH Tissue 1-Plex Assay (Thermo Fisher Scientific, Cat. No. QVT0051) with custom designed APP siRNA antisense guide strand probe set (Thermo Fisher), according to manufacturer's protocol, with Fast Red Substrate. The representative digital images were generated using a Zeiss Axio Observer microscope (Zeiss, Thornwood, NY, USA) under brightfield or fluorescent field.

ViewRNA results show uniform distribution of APP siRNA guide strand SSR-0106222 in regions of Cortex, Hippocampus, Striatum, Cerebellum, Brainstem (MidBrain and Medulla), 8 weeks after a single ICV injection at P0. While guide strand SSR-0106223 appears less effective in some regions. DSR-0103299 (Passenger strand SSR-0106206) treated animal shows reduced distribution (detectible) of the guide strand SSR-0106222 in all regions of mouse brain at 8 weeks. APP siRNA guide strand broadly distributed and retained within cytoplasm of neuron cells in these target regions of mouse brain at 8 weeks; siRNA passenger strand was also detectible, although at levels less than Guide strand, in neuron cells for up to 8 weeks. Lipid conjugate (DSR-0103301) on passenger PN showed no benefit under these conditions.

Various siRNAs for APP protein knockdown in brain tissues were designed and constructed. A number of siRNAs were tested in situ using an Immunohistochemistry (IHC) Assay. Example protocol for Immunohistochemistry Assay: Mouse brain hemisphere biopsies were fixed and embedded in paraffin as described above. 10 um sections were cut and followed by standard IHC staining methods using anti-APP Rabbit mAb #19389 (E3F3P) and isotype control Rabbit mAb #3900 (DA1E) (Cell Signaling Technology), followed by Polymer-HRP 2nd Detection antibody, and developed with DAB (brown) for 10 min and counterstained with hematoxylin (blue). The representative digital images were generated using a Zeiss Axio Observer microscope (Zeiss, Thornwood, NY, USA) under brightfield.

IHC results show specific APP protein staining in all brain regions of the mouse in PBS treated animals. DSR-0103199, DSR-0103216, DSR-0103300 and DSR-0103301 showed marked reduction of APP protein staining in frontal cortex while DSR-0103299 showed reduced APP protein staining, but not as strong as other siRNAs in frontal cortex. All siRNA showed reduction of APP proteins in some areas of striatum, all areas of hippocampus (CA1-CA4), all areas of cerebellum including Purkinje cells, and mid brain regions of brain stem. The extend of APP protein knock down is less obvious for DSR-01013216 and DSR-0103299 in medulla region of brain stem, while DSR-0103199, DSR-0103300 and DSR-0103301 have profound APP protein reduction in the similar regions in medulla.

Various siRNAs for APP protein knockdown in brain tissues were designed and constructed. Histopathology analysis was conducted on a number of siRNAs tested in situ. Sagittal half brain samples were fixed in 10% neutral buffered formalin. Samples were grossed, embedded in paraffin, trimmed at 4-6 μm thickness, and stained with hematoxylin and eosin. Histopathology assessment of sagittal brain sections obtained from 7 to 8-week-old male B6 mice administered single dose of DSR-0103199, DSR-0103216, DSR-0103299, DSR-0103300, or DSR-0103301 at 100 μg and PBS as a control resulted in no test-article or adverse findings. Bacterial meningoencephalitis in 1 mouse administered DSR-0103300 was present and was due to contamination of administration site that resulted in increased serum NFL levels in this animal. Procedure-related cannula track was present in 1 each out of 2 PBS (control animals), DSR-0103216, or DSR-0103301 administered animals and 2 out of 2 animals in DSR-0103199 and DSR-0103299 groups. These changes were chronic and had healed except for a control animal with an active neurodegenerative process that contributed to increased serum NFL levels.

### Example 8. Provided Oligonucleotides and Compositions are Tested in In Vitro BJAB and PBMC Cytokine Assay

BJAB cells (a human Burkitt lymphoma B cell line) or human peripheral blood mononuclear cells (PBMCs) were incubated at a density of 4×10⁵ cells/well for 24 hours with test articles in a 96-well plate. BJAB cells were treated with a 4-point curve (1, 3, 10 and 30 μM), while PBMCs were treated with a 6-point curve (0.1, 0.3, 1, 3, 10 and 30 μM). After 24 hours, the supernatant was transferred and the presence of cytokines was assessed using a commercial cytokine magnetic bead kit (EMD Millipore, catalog number HCYTOMAG-60K). The procedure was carried out in accordance with the manufacturer's instructions and the samples were acquired on the FLEXMAP 3D® system. Median fluorescent intensity was analyzed using a 5-parameter logistic curve-fitting method to calculate analyte concentrations in the test samples based on standard curves.

Average fold changes vs. PBS were calculated per cytokine by dividing average cytokine concentration per condition by average cytokine concentration for PBS-treated wells. For the PBMC experiment, fold changes were calculated per donor. Cytokine fold changes were Z-standardized by subtracting the mean Cytokine fold change and dividing the centered values by the standard deviation of the fold changes. For PBMC experiments, Z standardization was performed separately per donor. For each compound, the maximum responding concentration was identified per donor/cell line as the concentration at which the sum of the Z-standardized Cytokine fold changes was highest. For ranking of pro-inflammatory potential, Euclidean distance vs. PBS was calculated for each compound's maximal responding concentration per donor/cell line across Z-standardized Cytokine fold changes. For the BJAB experiment, compounds were ranked according to Euclidean distance vs. PBS. For the PBMC experiment, compounds were first ranked separately by donor according to Euclidean distance vs. PBS. The compound with the highest overall distance vs. PBS across donors was identified as the one in which the sum of the individual donor ranks was highest. To normalize distance values per donor, Euclidean distances were scaled per donor relative to that respective donor's Nusinersen distance (0%) and its distance for the compound with the highest overall distance vs. PBS (100%). Final rankings for the PBMC experiment were assigned based on the average scaled Euclidean distance vs. PBS across all donors.

Table 62. shows euclidian distance from PBS in BJAB cytokine assay.

| Compound ID | Distance vs PBS | Rank |
|---|---|---|
| DSR-0103199 | 0.0107 | 1 |
| DSR-0103216 | 0.0705 | 2 |
| ASO-0104323 | 0.0967 | 3 |
| DSR-0103300 | 0.1644 | 4 |
| DSR-0103299 | 0.2072 | 5 |
| DSR-0103301 | 0.2142 | 6 |
| ASO-0109259 | 0.4144 | 7 |
| ASO-0100157 | 1.9408 | 8 |
| ASO-0136356 | 9.0727 | 9 |

Table 63. shows euclidian distance from PBS in PBMC cytokine assay.

| | | | | Mean | |
|---|---|---|---|---|---|
| | Donor | Distance | Distance | (Distance | Rank |
| Compound ID | ID | vs PBS | Scaled | Scaled) | Mean |
| DSR-0103301 | 201563 | 0.5136 | −7.9161 | −3.0687 | 1 |
| DSR-0103301 | 201648 | 0.8717 | 0.6655 | −3.0687 | 1 |
| DSR-0103301 | 201653 | 1.3897 | −1.9555 | −3.0687 | 1 |
| ASO-0104323 | 201563 | 1.1614 | 0.0000 | 0.0000 | 2 |
| ASO-0104323 | 201648 | 0.8061 | 0.0000 | 0.0000 | 2 |
| ASO-0104323 | 201653 | 1.5957 | 0.0000 | 0.0000 | 2 |
| DSR-0103216 | 201563 | 1.1857 | 0.2968 | 3.4977 | 3 |
| DSR-0103216 | 201648 | 0.6761 | −1.3182 | 3.4977 | 3 |
| DSR-0103216 | 201653 | 2.8084 | 11.5144 | 3.4977 | 3 |
| DSR-0103300 | 201563 | 2.8519 | 20.6573 | 3.7459 | 4 |
| DSR-0103300 | 201648 | 0.5660 | −2.4354 | 3.7459 | 4 |
| DSR-0103300 | 201653 | 0.8600 | −6.9842 | 3.7459 | 4 |
| DSR-0103299 | 201563 | 2.1014 | 11.4867 | 4.0156 | 5 |
| DSR-0103299 | 201648 | 0.9080 | 1.0340 | 4.0156 | 5 |
| DSR-0103299 | 201653 | 1.5458 | −0.4738 | 4.0156 | 5 |
| DSR-0103199 | 201563 | 6.2669 | 62.3872 | 22.1240 | 6 |
| DSR-0103199 | 201648 | 0.9124 | 1.0784 | 22.1240 | 6 |
| DSR-0103199 | 201653 | 1.9018 | 2.9064 | 22.1240 | 6 |
| ASO-0109259 | 201563 | 4.2381 | 37.5963 | 31.4447 | 7 |
| ASO-0109259 | 201648 | 4.4812 | 37.2854 | 31.4447 | 7 |
| ASO-0109259 | 201653 | 3.6445 | 19.4523 | 31.4447 | 7 |
| ASO-0100157- | 201563 | 8.5738 | 90.5770 | 77.9365 | 8 |
| 004 | | | | | |
| ASO-0100157 | 201648 | 9.4751 | 87.9503 | 77.9365 | 8 |
| ASO-0100157 | 201653 | 7.4183 | 55.2822 | 77.9365 | 8 |
| ASO-0136356 | 201563 | 9.3450 | 100.0000 | 100.0000 | 9 |
| ASO-0136356 | 201648 | 10.6628 | 100.0000 | 100.0000 | 9 |
| ASO-0136356 | 201653 | 12.1283 | 100.0000 | 100.0000 | 9 |

### Example 9. Provided Oligonucleotides and Compositions are Active In Vitro in Mouse Primary Hepatocytes

Various siRNAs for mouse TTR were designed and constructed. A number of siRNAs were tested in vitro in mouse primary hepatocytes at one or a range of concentrations. Some siRNAs were also tested in mice (e.g., C57BL6 wild type mice).

Example protocol for in vitro determination of siRNA activity in mouse primary hepatocytes: For determination of siRNAs activity, siRNAs at specific concentration were gymnotically delivered to mouse primary hepatocytes plated at 96-well plates, with 10,000 cells/well. Following 48 hours treatment, total RNA was extracted using SV96 Total RNA Isolation kit (Promega). cDNA production from RNA samples were performed using High-Capacity cDNA Reverse Transcription kit (Thermo Fisher) following manufacturer's instructions and qPCR analysis performed in CFX System using iQ Multiplex Powermix (Bio-Rad). For mouse TTR mRNA, the following qPCR assay were utilized: IDT PrimeTime predesigned qPCR Assay Mm.PT.58.11922308. Mouse HPRT was used as normalizer (Forward 5′CAAACTTTGCTTTCCCTGGTT3′, Reverse 5′TGGCCTGTATCCAACACTTC3′, Probe 5′/5HEX/ACCAGCAAG/Zen/CTTGCAACCTTAACC/3IABkFQ/3′. mRNA knockdown levels were calculated as % mRNA remaining relative to mock treatment.

Table 64. shows % mouse TTR mRNA remaining (at 500, 150 and 50 μM siRNA treatment) in primary mouse hepatocytes relative to mouse HPRT control. N=2. N.D.: Not determined.

| | 500 pM | | | 150 pM | | |
|---|---|---|---|---|---|---|
| | % remaining | % remaining | | % remaining | % remaining | |
| | mRNA (mTTR/ | mRNA (mTTR/ | | mRNA (mTTR/ | mRNA (mTTR/ | |
| siRNA | mHPRT)-1 | mHPRT)-2 | Mean | mHPRT)-1 | mHPRT)-2 | Mean |
| DSR-0102524 | 32.28 | 24.27 | 28.27 | 67.91 | 46.56 | 57.24 |
| DSR-0102626 | 36.38 | 18.77 | 27.58 | 53.31 | 33.71 | 43.51 |
| DSR-0103360 | 32.67 | 23.34 | 28.01 | 59.74 | 59.18 | 59.46 |
| DSR-0103362 | 25.63 | 15.67 | 20.65 | 43.67 | 35.29 | 39.48 |
| DSR-0103361 | 20.01 | 13.79 | 16.9 | 50.24 | 43.74 | 46.99 |
| DSR-0103359 | 15.78 | 12.43 | 14.11 | 59.69 | 41.97 | 50.83 |

| | 50 pM | | |
|---|---|---|---|
| | % remaining | % remaining | |
| | mRNA (mTTR/ | mRNA (mTTR/ | |
| siRNA | mHPRT)-1 | mHPRT)-2 | Mean |
| DSR-0102524 | 77.27 | 63.16 | 70.21 |
| DSR-0102626 | 83.12 | 63.14 | 73.13 |
| DSR-0103360 | 95.39 | 84.64 | 90.01 |
| DSR-0103362 | 65.87 | 57.07 | 61.47 |
| DSR-0103361 | 44.22 | 61.16 | 52.69 |
| DSR-0103359 | 50.86 | 82.76 | 66.81 |

Table 65. shows IC50 of knocking down mouse TTR mRNA in mouse primary hepatocyte.

| siRNA | IC50 (pM) | 95% CI |
|---|---|---|
| DSR-0103028 | 62.88 | 46.20 to 85.94 |
| DSR-0102626 | 57.87 | 38.64 to 87.34 |
| DSR-0103361 | 52.80 | 29.38 to 95.40 |

### Example 10. Provided Oligonucleotides and Compositions are Active In Vivo

In vivo determination of mouse TTR siRNA activity: All animal procedures were performed under IACUC guidelines. To evaluate the potency and liver exposure of provided oligonucleotides and compositions, male 8-10 weeks of age C57BL/6 mice were dose at 0.5 mg/kg at desired oligonucleotide concentration on Day 0 by subcutaneous administration. Animals were euthanized on Day 7 by CO₂ asphyxiation followed by thoracotomy and terminal blood collection. After cardiac perfusion with PBS, liver samples were harvested and flash-frozen in dry ice. Liver total RNA was extracted using SV96 Total RNA Isolation kit (Promega), after tissue lysis with TRIzol and bromochloropropane. cDNA production from RNA samples were performed using High-Capacity cDNA Reverse Transcription kit (Thermo Fisher) following manufacturer's instructions and qPCR analysis performed in CFX System using iQ Multiplex Powermix (Bio-Rad). For mouse TTR mRNA, the following qPCR assay were utilized: IDT Taqman qPCR assay ID Mm.PT.58.11922308. Oligonucleotide accumulation in liver was determined by hybrid ELISA.

To evaluate the durability of provided oligonucleotides and compositions, male 8-10 weeks of age C₅₇BL/6 mice were dose at 0.5 mg/kg at desired oligonucleotide concentration on Day 0 by subcutaneous administration. On Day 0 (pre-dose) and then weekly, whole blood was collected via submandibular bleeding into serum separator tubes, and processed serum samples were kept at −70° C. Mouse TTR protein concentration in the serum was assessed using the Mouse Prealbumin ELISA kit (Crystal Chem) and following manufacturer's instructions.

Ago2 immunoprecipitation assay: Tissues were lysed in lysis buffer 50 mM Tris-HCl at pH 7.5, 200 mM NaCl, 0.5% Triton X-100, 2 mM EDTA, 1 mg/mL heparin) with protease inhibitor (Sigma-Aldrich). Lysate concentration was measured with a protein BCA kit (Pierce BCA protein assay kit or Bradford protein assay kit). Anti-Ago2 antibody was purchased from Wako Chemicals. Control mouse IgG was from eBioscience. Dynabeads (Invitrogen) were used to precipitate antibodies. Ago2-associated siRNA and endogenous miR22 were measured by Stem-Loop RT followed by TaRMan PCR analysis using Taqman miRNA and siRNA assay kit (Thermo fisher) based on manufacturer's methods.

Table 66. shows 0% mouse TTR mRNA remaining relative to PBS control. N=5. N.D.: Not determined.

| PBS | | DSR-0102657 | | DSR-0102626 | | DSR-0103359 | |
|---|---|---|---|---|---|---|---|
| | % remaining | | % remaining | | % remaining | | % remaining |
| animal | of mTTR | animal | of mTTR | animal | of mTTR | animal | of mTTR |
| No. | mRNA | No. | mRNA | No. | mRNA | No. | mRNA |
| 1 | 86.94 | 6 | 93.70 | 11 | 8.06 | 16 | 22.76 |
| 2 | 107.68 | 7 | 111.90 | 12 | 20.69 | 17 | 12.89 |
| 3 | 101.18 | 8 | 134.51 | 13 | 14.45 | 18 | 10.79 |
| 4 | 111.85 | 9 | 108.31 | 14 | 9.28 | 19 | 18.96 |
| 5 | 92.36 | 10 | 123.57 | 15 | 10.49 | 20 | 18.74 |
| Mean | 100 | Mean | 114.40 | Mean | 12.59 | Mean | 16.83 |

| | | |
|---|---|---|
| DSR-0103360 | DSR-0103361 | DSR-0103362 |

| | % remaining | | % remaining | | % remaining |
|---|---|---|---|---|---|
| animal | of mTTR | animal | of mTTR | animal | of mTTR |
| No. | mRNA | No. | mRNA | No. | mRNA |
| 21 | 28.40 | 26 | 9.40 | 31 | 10.50 |
| 22 | 23.51 | 27 | 15.91 | 32 | 26.17 |
| 23 | 24.40 | 28 | 18.63 | 33 | 4.86 |
| 24 | 21.07 | 29 | 23.16 | 34 | 21.58 |
| 25 | 38.78 | 30 | 14.89 | 35 | 3.42 |
| Mean | 27.23 | Mean | 16.40 | Mean | 13.31 |

Table 67. shows % mouse TTR protein remaining relative to PBS control. N=5. N.D.: Not Determined.

| | PBS | | | | | |
|---|---|---|---|---|---|---|
| Day | animal1 | animal2 | animal3 | animal4 | animal5 | Mean |
| 0 | 108 | 105 | 71 | 118 | 98 | 100 |
| 7 | 103 | 98 | 80 | 114 | 106 | 100 |
| 14 | 93 | 99 | 88 | 120 | 100 | 100 |
| 21 | 101 | 99 | 90 | 102 | 108 | 100 |
| 28 | 134 | 82 | 89 | 105 | 91 | 100 |
| 35 | 105 | 93 | 94 | 110 | 99 | 100 |
| 42 | 101 | 83 | 109 | 115 | 92 | 100 |
| 49 | 110 | 100 | 108 | 87 | 94 | 100 |

| | DSR-0102657, 0.5 mg/kg | | | | | |
|---|---|---|---|---|---|---|
| Day | animal6 | animal7 | animal8 | animal9 | animal10 | Mean |
| 0 | 81 | 59 | 121 | 62 | 99 | 84 |
| 7 | 86 | 68 | 112 | 58 | 89 | 83 |
| 14 | 91 | 87 | 109 | 86 | 105 | 96 |
| 21 | 69 | 89 | 95 | 72 | 100 | 85 |
| 28 | 88 | 86 | 102 | 83 | 92 | 90 |
| 35 | 84 | 502 | 95 | 72 | 92 | 169 |
| 42 | 80 | 110 | 72 | 60 | 87 | 82 |
| 49 | 66 | 95 | 89 | 74 | 86 | 82 |

| | DSR-0102626, 0.5 mg/kg | | | | | |
|---|---|---|---|---|---|---|
| Day | animal11 | animal12 | animal13 | animal14 | animal15 | Mean |
| 0 | 88 | 99 | 103 | 122 | 102 | 103 |
| 7 | 7 | 7 | 8 | 9 | 6 | 8 |
| 14 | 14 | 12 | 15 | 11 | 10 | 12 |
| 21 | 23 | 24 | 26 | 19 | 21 | 22 |
| 28 | 38 | 30 | 31 | 22 | 32 | 31 |
| 35 | 54 | 54 | 55 | 46 | 55 | 53 |
| 42 | 80 | 68 | 70 | 72 | 88 | 76 |
| 49 | 81 | 73 | 77 | 75 | 85 | 78 |
| | DSR-0103359, 0.5 mg/kg | | | | | |
| Day | animal16 | animal17 | animal18 | animal19 | animal20 | Mean |
| 0 | 160 | 94 | 97 | 77 | 95 | 104 |
| 7 | 12 | 15 | 15 | 14 | 20 | 15 |
| 14 | 12 | 17 | 13 | 9 | 22 | 15 |
| 21 | 15 | 25 | 24 | 16 | 26 | 21 |
| 28 | 17 | 36 | 22 | 22 | 31 | 26 |
| 35 | 25 | 43 | 41 | 33 | 39 | 36 |
| 42 | 38 | 44 | 61 | 55 | 53 | 50 |
| 49 | 59 | 58 | 73 | 67 | 63 | 64 |
| | DSR-0103360, 0.5 mg/kg | | | | | |
| Day | animal21 | animal22 | animal23 | animal24 | animal25 | Mean |
| 0 | 86 | 79 | 84 | 98 | 91 | 88 |
| 7 | 13 | 16 | 11 | 13 | 10 | 12 |
| 14 | 14 | 22 | 14 | 17 | 8 | 15 |
| 21 | 15 | 22 | 15 | 17 | 12 | 16 |
| 28 | 20 | 31 | 19 | 27 | 16 | 23 |
| 35 | 31 | 37 | 27 | 38 | 25 | 32 |
| 42 | 39 | 47 | 41 | 50 | 32 | 42 |
| 49 | 53 | 49 | 44 | 66 | 62 | 55 |
| | DSR-0103361, 0.5 mg/kg | | | | | |
| Day | animal26 | animal27 | animal28 | animal29 | animal30 | Mean |
| 0 | 88 | 80 | 96 | 88 | 85 | 87 |
| 7 | 7 | 8 | 6 | 8 | 10 | 8 |
| 14 | 6 | 5 | 5 | 7 | 4 | 6 |
| 21 | 10 | 9 | 13 | 12 | 14 | 11 |
| 28 | 10 | 7 | 16 | 16 | 17 | 13 |
| 35 | 18 | 11 | 30 | 25 | 3 | 18 |
| 42 | 33 | 23 | 51 | 35 | 48 | 38 |
| 49 | 40 | 27 | 60 | 59 | 69 | 51 |
| | DSR-0103362, 0.5 mg/kg | | | | | |
| Day | animal31 | animal32 | animal33 | animal34 | animal35 | Mean |
| 0 | 86 | 70 | 85 | 91 | 97 | 86 |
| 7 | 16 | 13 | 9 | 14 | 14 | 13 |
| 14 | 14 | 15 | 7 | 14 | 13 | 13 |
| 21 | 13 | 23 | 14 | 16 | 15 | 16 |
| 28 | 18 | 32 | 15 | 15 | 21 | 20 |
| 35 | 26 | 38 | 29 | 22 | 29 | 29 |
| 42 | 28 | 50 | 37 | 25 | 42 | 36 |
| 49 | 44 | 60 | 59 | 44 | 71 | 56 |

Table 68. shows Ago 2 loading of guide strand relative to miR-122 at day 7. N=3.

| | Ct: | Ct: | Ct: | Ct: | Relative |
|---|---|---|---|---|---|
| | mTTR/ | miR- | mTTR/ | miR- | mTTR/ |
| | Ago2 | 122/Ago2 | IgG | 122/IgG | miR122 |
| PBS-1 | 36.77 | 21.2 | 35.93 | 28.23 | 0.0043 |
| PBS-2 | 34.14 | 18.7 | 37.45 | 29.61 | 0.0052 |
| PBS-3 | 34.54 | 18.92 | 35.8 | 29.57 | 0.0015 |
| DSR- | 30.56 | 17.97 | 37.54 | 28.24 | 0.1022 |
| 0102626-1 | | | | | |
| DSR- | 29.70 | 17.85 | 34.63 | 28.24 | 0.0227 |
| 0102626-2 | | | | | |
| DSR- | 30.58 | 17.58 | 34.1 | 27.74 | 0.0100 |
| 0102626-3 | | | | | |
| DSR- | 31.33 | 17.26 | 34.12 | 27.23 | 0.0069 |
| 0103359-1 | | | | | |
| DSR- | 30.21 | 17.36 | 33.55 | 27.86 | 0.0070 |
| 0103359-2 | | | | | |
| DSR- | 30.17 | 17.07 | 34.15 | 27.24 | 0.0137 |
| 0103359-3 | | | | | |
| DSR- | 32.66 | 17.51 | 34.26 | 28.29 | 0.0017 |
| 0103360-1 | | | | | |
| DSR- | 34.13 | 18.86 | 34.39 | 27.73 | 0.0026 |
| 0103360-2 | | | | | |
| DSR- | 33.27 | 17.18 | 34.32 | 27.95 | 0.0012 |
| 0103360-3 | | | | | |
| DSR- | 33.15 | 18.46 | 35.9 | 29.02 | 0.0045 |
| 0103361-1 | | | | | |
| DSR- | 33.36 | 18.76 | 33.91 | 28.58 | 0.0016 |
| 0103361-2 | | | | | |
| DSR- | 32.16 | 17.88 | 35.62 | 28.65 | 0.0063 |
| 0103361-3 | | | | | |
| DSR- | 29.66 | 17.22 | 33.94 | 27.65 | 0.0141 |
| 0103362-1 | | | | | |
| DSR- | 29.13 | 17.43 | 34.14 | 27.78 | 0.0247 |
| 0103362-2 | | | | | |
| DSR- | 28.95 | 17.3 | 34.35 | 28.74 | 0.0152 |
| 0103362-3 | | | | | |

### Example 11. Provided Oligonucleotides and Compositions are Active In Vitro in Human iCell GABA Neurons

Various siRNAs for mouse APP were designed and constructed. A number of siRNAs were tested in vitro in human iCell GABA neurons at one or a range of concentrations. Some siRNAs were also tested in mice (e.g., C57BL6 wild type mice).

Example protocol for in vitro determination of siRNA activity in human iCell GABA neurons: For determination of siRNAs activity, siRNAs at specific concentration were gymnotically delivered to human iCell GABA neurons plated at 96-well plates, with 40,000 cells/well. Following 5 days treatment, total RNA was extracted using SV96 Total RNA Isolation kit (Promega). cDNA production from RNA samples were performed using High-Capacity cDNA Reverse Transcription kit (Thermo Fisher) following manufacturer's instructions and qPCR analysis performed in CFX System using iQ Multiplex Powermix (Bio-Rad). For human APP mRNA, the following qPCR assay were utilized: Thermofisher Taqman qPCR assay ID Hs00169098-ml. Human SFRS9 was used as normalizer (Forward, 5′-TGGAATATGCCCTGCGTAAA-3′; Reverse, 5′-TGGTGCTTCTCTCAGGATAAAC-3′, Probe, 5′-TGGATGACACCAAATTCCGCTCTCA-3′. mRNA knockdown levels were calculated as % mRNA remaining relative to mock treatment.

Table 69. shows % human APP mRNA remaining (at 3 and 1 uM siRNA treatment) in iCell GABA neurons relative to human SFRS9 control. N=2. N.D.: Not determined.

| | 3 uM | | | 1 uM | | |
|---|---|---|---|---|---|---|
| | % remaining | % remaining | | % remaining | % remaining | |
| | mRNA (hAPP/ | mRNA (hAPP/ | | mRNA (hAPP/ | mRNA (hAPP/ | |
| siRNA | hSFRS9)- 1 | hSFRS9)-2 | Mean | hSFRS9)- 1 | hSFRS9)-2 | Mean |
| DSR-0103190 | 61.00 | 60.40 | 60.70 | 70.70 | 87.30 | 79.00 |
| DSR-0103164 | 12.90 | 14.40 | 13.65 | 27.20 | 27.90 | 27.55 |
| DSR-0103210 | 53.20 | 50.00 | 51.60 | 60.90 | 74.20 | 67.55 |
| DSR-0103211 | 60.70 | 64.40 | 62.55 | 88.90 | 73.90 | 81.40 |
| DSR-0103206 | 13.70 | 17.40 | 15.55 | 32.10 | 46.90 | 39.50 |
| DSR-0103217 | 27.90 | 35.10 | 31.50 | 66.10 | 61.90 | 64.00 |
| DSR-0103223 | 14.90 | 17.00 | 15.95 | 30.70 | 45.60 | 38.15 |
| DSR-0103224 | 28.40 | 78.60 | 53.50 | 61.20 | 66.20 | 63.70 |
| DSR-0103225 | 16.70 | 22.20 | 19.45 | 47.20 | 63.20 | 55.20 |
| DSR-0103218 | 26.30 | 33.50 | 29.90 | 79.00 | 64.60 | 71.80 |

Table 70. shows % human APP mRNA remaining (at 3 and 1 uM siRNA treatment) in iCell GABA neurons relative to human SFRS9 control. N=2. N.D.: Not determined.

| | 3 uM | | | 1 uM | | |
|---|---|---|---|---|---|---|
| | % remaining | % remaining | | % remaining | % remaining | |
| | mRNA (hAPP/ | mRNA (hAPP/ | | mRNA (hAPP/ | mRNA (hAPP/ | |
| siRNA | hSFRS9)- 1 | hSFRS9)-2 | Mean | hSFRS9)- 1 | hSFRS9)-2 | Mean |
| DSR-0103190 | 114.20 | 92.20 | 103.20 | 98.00 | 92.10 | 95.05 |
| DSR-0103342 | 77.70 | 66.90 | 72.30 | 74.10 | 72.30 | 73.20 |
| DSR-0103343 | 74.30 | 80.40 | 77.35 | 100.00 | 104.70 | 102.35 |
| DSR-0103408 | 14.90 | 15.10 | 15.00 | 47.90 | 54.10 | 51.00 |
| DSR-0103424 | 15.50 | 18.40 | 16.95 | 84.90 | 60.70 | 72.80 |
| DSR-0103414 | 17.60 | 19.30 | 18.45 | 53.80 | 49.50 | 51.65 |
| DSR-0103429 | 16.40 | 21.20 | 18.80 | 52.00 | 60.00 | 56.00 |
| DSR-0103411 | 18.90 | 24.40 | 21.65 | 50.80 | 57.60 | 54.20 |
| DSR-0103217 | 17.10 | 24.10 | 20.60 | 47.20 | 59.30 | 53.25 |
| DSR-0103206 | 11.30 | 12.50 | 11.90 | 30.40 | 31.70 | 31.05 |
| DSR-0103164 | 12.80 | 18.90 | 15.85 | 22.70 | 26.50 | 24.60 |
| DSR-0103416 | 14.90 | 16.40 | 15.65 | 30.90 | 28.30 | 29.60 |

Table 71. shows 0% human APP mRNA remaining (at 1, 0.04, and 0.008 uM siRNA treatment) in primary human hepatocytes relative to human SFRS9 control. N=2. N.D.: Not determined.

| | 1 uM | | | 0.04 uM | | | 0.008 uM | | |
|---|---|---|---|---|---|---|---|---|---|
| | % | % | | % | % | | % | % | |
| | remaining | remaining | | remaining | remaining | | remaining | remaining | |
| | mRNA | mRNA | | mRNA | mRNA | | mRNA | mRNA | |
| | (hAPP/ | (hAPP/ | | (hAPP/ | (hAPP/ | | (hAPP/ | (hAPP/ | |
| siRNA | hSFRS9) − 1 | hSFRS9) − 2 | Mean | hSFRS9) − 1 | hSFRS9) − 2 | Mean | hSFRS9) − 1 | hSFRS9) − 2 | Mean |
| DSR- | 6.08 | 5.70 | 5.89 | 23.85 | 23.17 | 23.51 | 57.65 | 54.43 | 56.04 |
| 0103301 | | | | | | | | | |
| DSR- | 7.33 | 7.06 | 7.19 | 13.98 | 15.65 | 14.82 | 40.10 | 43.25 | 41.67 |
| 0103300 | | | | | | | | | |
| DSR- | 11.94 | 12.26 | 12.10 | 31.40 | 31.31 | 31.35 | 58.22 | 55.42 | 56.82 |
| 0103299 | | | | | | | | | |
| DSR- | 8.12 | 6.01 | 7.06 | 17.73 | 18.47 | 18.10 | 43.00 | 41.26 | 42.13 |
| 0103216 | | | | | | | | | |
| DSR- | 7.78 | 6.44 | 7.11 | 15.04 | 18.77 | 16.90 | 39.37 | 39.18 | 39.27 |
| 0103199 | | | | | | | | | |

Table 72. shows IC50 of knocking down human APP mRNA in human iCell GABA neurons.

| siRNA | IC50 (uM) | 95% CI |
|---|---|---|
| DSR-0103199 | 0.32 | 0.16 to 0.64 |
| DSR-0103216 | 0.34 | 0.23 to 0.50 |
| DSR-0103299 | 0.63 | 0.45 to 0.88 |
| DSR-0103300 | 0.23 | 0.18 to 0.29 |
| DSR-0103301 | 0.61 | 0.51 to 0.73 |

Table 73. shows 0% human APP mRNA remaining (at 3 and 1 uM siRNA treatment) in iCell GABA neurons relative to human SFRS9 control. N=2. N.D.: Not determined.

| | 3 uM | | | 1 uM | | |
|---|---|---|---|---|---|---|
| | % remaining | % remaining | | % remaining | % remaining | |
| | mRNA | mRNA | | mRNA | mRNA | |
| | (hAPP/ | (hAPP/ | | (hAPP/ | (hAPP/ | |
| siRNA | hSFRS9) − 1 | hSFRS9) − 2 | Mean | hSFRS9) − 1 | hSFRS9) − 2 | Mean |
| DSR- | 15.80 | 14.40 | 15.10 | 23.10 | 31.30 | 27.20 |
| 0103415 | | | | | | |
| DSR- | 22.40 | 24.60 | 23.50 | 32.80 | 34.50 | 33.65 |
| 0103412 | | | | | | |
| DSR- | 42.70 | 38.60 | 40.65 | 61.70 | 79.40 | 70.55 |
| 0103719 | | | | | | |
| DSR- | 53.00 | 59.50 | 56.25 | 72.70 | 69.90 | 71.30 |
| 0103718 | | | | | | |

Table 74. shows 00 human APP mRNA remaining (at 3 and 1 uM siRNA treatment) in iCell GABA neurons relative to human SFRS9 control. N=2. N.D.: Not determined.

| | 3 uM | | | 1 uM | | |
|---|---|---|---|---|---|---|
| | % remaining | % remaining | | % remaining | % remaining | |
| | mRNA | mRNA | | mRNA | mRNA | |
| | (hAPP/ | (hAPP/ | | (hAPP/ | (hAPP/ | |
| siRNA | hSFRS9) − 1 | hSFRS9) − 2 | Mean | hSFRS9) − 1 | hSFRS9) − 2 | Mean |
| DSR- | 15.60 | 16.30 | 15.95 | 28.40 | 28.80 | 28.60 |
| 0103428 | | | | | | |
| DSR- | 19.90 | 15.80 | 17.85 | 28.50 | 30.80 | 29.65 |
| 0103427 | | | | | | |
| DSR- | 18.50 | 12.30 | 15.40 | 29.90 | 27.20 | 28.55 |
| 0103426 | | | | | | |
| DSR- | 20.00 | 18.00 | 19.00 | 33.60 | 33.30 | 33.45 |
| 0103409 | | | | | | |
| DSR- | 15.80 | 16.20 | 16.00 | 33.50 | 27.40 | 30.45 |
| 0103404 | | | | | | |
| DSR- | 37.60 | 35.80 | 36.70 | 55.00 | 66.80 | 60.90 |
| 0103420 | | | | | | |
| DSR- | 22.70 | 21.80 | 22.25 | 56.20 | 55.00 | 55.60 |
| 0103425 | | | | | | |
| DSR- | 22.20 | 27.20 | 24.70 | 71.60 | 67.60 | 69.60 |
| 0103419 | | | | | | |
| DSR- | 20.70 | 21.10 | 20.90 | 47.60 | 60.10 | 53.85 |
| 0103431 | | | | | | |
| DSR- | 20.70 | 20.50 | 20.60 | 52.00 | 68.20 | 60.10 |
| 0103418 | | | | | | |
| DSR- | 22.70 | 18.10 | 20.40 | 51.30 | 51.30 | 51.30 |
| 0103430 | | | | | | |
| DSR- | 12.80 | 18.90 | 15.85 | 22.70 | 26.50 | 24.60 |
| 0103164 | | | | | | |
| DSR- | 14.90 | 16.40 | 15.65 | 30.90 | 28.30 | 29.60 |
| 0103416 | | | | | | |

Table 75. shows % human APP mRNA remaining (at 3 and 1 uM siRNA treatment) in iCell GABA neurons relative to human SFRS9 control. N=2. N.D.: Not determined.

| | 3 uM | | | 1 uM | | |
|---|---|---|---|---|---|---|
| | % remaining | % remaining | | % remaining | % remaining | |
| | mRNA | mRNA | | mRNA | mRNA | |
| | (hAPP/ | (hAPP/ | | (hAPP/ | (hAPP/ | |
| siRNA | hSFRS9) − 1 | hSFRS9) − 2 | Mean | hSFRS9) − 1 | hSFRS9) − 2 | Mean |
| DSR- | 15.90 | 14.60 | 15.25 | 26.10 | 31.30 | 28.70 |
| 0103717 | | | | | | |
| DSR- | 15.60 | 13.70 | 14.65 | 27.50 | 23.10 | 25.30 |
| 0103716 | | | | | | |
| DSR- | 14.30 | 14.50 | 14.40 | 32.00 | 38.00 | 35.00 |
| 0103715 | | | | | | |
| DSR- | 14.80 | 15.20 | 15.00 | 30.20 | 31.00 | 30.60 |
| 0103714 | | | | | | |
| DSR- | 17.20 | 17.60 | 17.40 | 30.60 | 39.70 | 35.15 |
| 0103713 | | | | | | |
| DSR- | 13.80 | 15.20 | 14.50 | 28.10 | 25.40 | 26.75 |
| 0103712 | | | | | | |
| DSR- | 31.70 | 26.70 | 29.20 | 45.70 | 52.10 | 48.90 |
| 0103325 | | | | | | |

### Example 12. Provided Oligonucleotides and Compositions are Active In Vivo

All animal procedures were performed under IACUC guidelines. To evaluate the potency and liver exposure of provided oligonucleotides and compositions, male 8-10 weeks of age C57BL/6 mice were dose at 50 mg/kg on Day 0 by subcutaneous administration. Animals were euthanized on Day 7 or 28 by CO2 asphyxiation followed by thoracotomy and terminal blood collection. After cardiac perfusion with PBS, various tissue samples were harvested and flash-frozen in dry ice. Total RNA was extracted using RNeasy 96 kit (Qiagen), after tissue lysis with TRIzol and chloroform. cDNA production from RNA samples were performed using High-Capacity cDNA Reverse Transcription kit (Thermo Fisher) following manufacturer's instructions and qPCR analysis performed in CFX System using iQ Multiplex Powermix (Bio-Rad for mouse APP mRNA, the following qPCR assay were utilized: Thermofisher Taqman qPCR assay ID Mm01344172_m1. Mouse HPRT was used as normalizer (Forward 5′CAAACTTTGCTTTCCCTGGTT3′, Reverse 5′TGGCCTGTATCCAACACTTC3′, Probe 5′/5HEX/ACCAGCAAG/Zen/CTTGCAACCTTAACC/3IABkFQ/3′. Oligonucleotide accumulation was determined by sandwich ELISA. Serum biomarkers were measured at Charles River Laboratory.

Ago2 immunoprecipitation assay: Tissues were lysed in lysis buffer 50 mM Tris-HCl at pH 7.5, 200 mM NaCl, 0.5% Triton X-100, 2 mM EDTA, 1 mg/mL heparin) with protease inhibitor (Sigma-Aldrich). Lysate concentration was measured with a protein BCA kit (Pierce BCA protein assay kit or Bradford protein assay kit). Anti-Ago2 antibody was purchased from Wako Chemicals. Control mouse IgG was from eBioscience. Dynabeads (Invitrogen) were used to precipitate antibodies. Ago2-associated siRNA and endogenous miR122 or miR143 were measured by Stem-Loop RT followed by TaqMan PCR analysis using Taqman miRNA and siRNA assay kit (Thermo fisher) based on manufacturer's methods.

Table 76. shows % mouse APP mRNA remaining relative to PBS control in the kidney 7 days post dosing. N=5. N.D.: Not determined.

| | % remaining of mAPP mRNA | | | | | |
|---|---|---|---|---|---|---|
| animal No. | PBS | DSR-0103199 | DSR-0103216 | DSR-0103299 | DSR-0103300 | DSR-0103301 |
| 1 | 85.36 | 122.85 | 70.14 | 57.26 | 51.35 | 47.02 |
| 2 | 99.41 | 70.02 | 73.48 | 61.90 | 48.81 | 49.16 |
| 3 | 122.58 | 67.48 | 60.40 | 50.00 | 58.70 | 56.62 |
| 4 | 114.39 | 122.01 | 76.33 | 51.77 | 46.71 | 53.10 |
| 5 | 78.26 | 68.62 | 57.15 | 64.94 | 52.17 | 49.27 |
| Mean | 100.00 | 90.19 | 67.50 | 57.17 | 51.55 | 51.03 |

Table 77. shows % mouse APP mRNA remaining relative to PBS control in the lung 7 days post dosing. N=5. N.D.: Not determined.

| | % remaining of mAPP mRNA | | | | | |
|---|---|---|---|---|---|---|
| animal No. | PBS | DSR-0103199 | DSR-0103216 | DSR-0103299 | DSR-0103300 | DSR-0103301 |
| 1 | 118.57 | 86.19 | 61.17 | 83.76 | 93.37 | 84.73 |
| 2 | 88.93 | 86.56 | 80.45 | 90.96 | 83.56 | 71.78 |
| 3 | 108.52 | 107.77 | 102.05 | 84.27 | 76.72 | 64.47 |
| 4 | 75.01 | 139.34 | 118.34 | 73.79 | 105.69 | 79.28 |
| 5 | 108.97 | 117.36 | 52.35 | 65.36 | 81.96 | 68.27 |
| Mean | 100.00 | 107.44 | 82.87 | 79.63 | 88.26 | 73.71 |

Table 78. shows 00 mouse APP mRNA remaining relative to PBS control in the quad 7 days post dosing. N=5. N.D.: Not determined.

| | % remaining of mAPP mRNA | | | | | |
|---|---|---|---|---|---|---|
| animal No. | PBS | DSR-0103199 | DSR-0103216 | DSR-0103299 | DSR-0103300 | DSR-0103301 |
| 1 | 63.45 | 63.97 | 36.05 | 65.64 | 51.59 | 31.96 |
| 2 | 78.76 | 74.49 | 48.73 | 45.92 | 45.36 | 59.03 |
| 3 | 121.49 | 75.26 | 56.15 | 45.70 | 66.45 | 64.54 |
| 4 | 134.75 | 48.11 | 51.88 | 48.18 | 72.16 | 66.54 |
| 5 | 101.55 | 61.49 | 47.66 | 69.01 | 79.49 | 60.32 |
| Mean | 100.00 | 64.67 | 48.09 | 54.89 | 63.01 | 56.48 |

Table 79. shows 00 mouse APP mRNA remaining relative to PBS control in the spleen 7 days post dosing. N=5. N.D.: Not determined.

| | % remaining of mAPP mRNA | | | | | |
|---|---|---|---|---|---|---|
| animal No. | PBS | DSR-0103199 | DSR-0103216 | DSR-0103299 | DSR-0103300 | DSR-0103301 |
| 1 | 86.01 | 100.17 | 85.40 | 107.31 | 86.87 | 97.31 |
| 2 | 96.23 | 115.38 | 97.66 | 130.66 | 89.36 | 107.61 |
| 3 | 113.18 | 83.79 | 73.64 | 89.08 | 99.30 | 91.44 |
| 4 | 87.72 | 144.33 | 93.30 | 124.44 | 122.73 | 99.96 |
| 5 | 116.86 | 84.92 | 86.15 | 160.76 | 132.16 | 112.01 |
| Mean | 100.00 | 105.72 | 87.23 | 122.45 | 106.08 | 101.67 |

Table 80. shows % mouse APP mRNA remaining relative to PBS control in the liver 7 days post dosing. N=5. N.D.: Not determined.

| | % remaining of mAPP mRNA | | | | | |
|---|---|---|---|---|---|---|
| animal No. | PBS | DSR-0103199 | DSR-0103216 | DSR-0103299 | DSR-0103300 | DSR-0103301 |
| 1 | 163.61 | 23.56 | 25.82 | 27.03 | 30.78 | 24.75 |
| 2 | 76.61 | 27.91 | 30.56 | 39.24 | 29.70 | 35.40 |
| 3 | 129.52 | 44.78 | 38.09 | 28.04 | 25.62 | 28.29 |
| 4 | 76.48 | 41.92 | 23.95 | 27.55 | 26.39 | 27.33 |
| 5 | 53.78 | 61.43 | 37.42 | 40.15 | 32.40 | 31.33 |
| Mean | 100.00 | 39.92 | 31.17 | 32.40 | 28.98 | 29.42 |

Table 81. shows % mouse APP mRNA remaining relative to PBS control in the white Adipose Tissue 7 days post dosing. N=5. N.D.: Not determined.

| | % remaining of mAPP mRNA | | | | | |
|---|---|---|---|---|---|---|
| animal No. | PBS | DSR-0103199 | DSR-0103216 | DSR-0103299 | DSR-0103300 | DSR-0103301 |
| 1 | 111.65 | 58.32 | 52.64 | 39.07 | 30.36 | 19.07 |
| 2 | 99.73 | 63.99 | 52.98 | 53.75 | 34.25 | 23.22 |
| 3 | 100.06 | 62.41 | 40.10 | 51.87 | 42.47 | 25.18 |
| 4 | 117.14 | 50.79 | 56.61 | 59.20 | 27.33 | 20.33 |
| 5 | 71.42 | 66.34 | 37.61 | 31.01 | 41.22 | 27.47 |
| Mean | 100.00 | 60.37 | 47.99 | 46.98 | 35.13 | 23.05 |

Table 82. shows % mouse APP mRNA remaining relative to PBS control in the diaphragm 7 days post dosing. N=5. N.D.: Not determined.

| | % remaining of mAPP mRNA | | | | | |
|---|---|---|---|---|---|---|
| animal No. | PBS | DSR-0103199 | DSR-0103216 | DSR-0103299 | DSR-0103300 | DSR-0103301 |
| 1 | 100.17 | 70.03 | 71.55 | 55.95 | 53.54 | 59.52 |
| 2 | 106.27 | 72.40 | 64.20 | 26.03 | 61.82 | 64.63 |
| 3 | 100.72 | 71.96 | 75.35 | 62.05 | 55.46 | 70.65 |
| 4 | 104.18 | 68.96 | 61.86 | 67.66 | 79.59 | 66.05 |
| 5 | 88.66 | 78.04 | 60.27 | 62.49 | 67.34 | 72.90 |
| Mean | 100.00 | 72.28 | 66.65 | 54.83 | 63.55 | 66.75 |

Table 83. shows 00 mouse APP mRNA remaining relative to PBS control in the heart 7 days post dosing. N=5. N.D.: Not determined.

| | % remaining of mAPP mRNA | | | | | |
|---|---|---|---|---|---|---|
| animal No. | PBS | DSR-0103199 | DSR-0103216 | DSR-0103299 | DSR-0103300 | DSR-0103301 |
| 1 | 96.13 | 87.50 | 87.10 | 111.69 | 83.24 | 122.16 |
| 2 | 98.95 | 89.23 | 68.59 | 110.87 | 145.74 | 111.76 |
| 3 | 100.66 | 147.70 | 81.57 | 118.51 | 86.36 | 86.66 |
| 4 | 102.65 | 107.22 | 100.47 | 63.14 | 90.02 | 89.57 |
| 5 | 101.61 | 160.56 | 62.98 | 79.63 | 74.25 | 117.66 |
| Mean | 100.00 | 118.44 | 80.14 | 96.77 | 95.92 | 105.56 |

Table 84. shows 00 mouse APP mRNA remaining relative to PBS control in the brown adipose tissue 7 days post dosing. N=5. N.D.: Not determined.

| | % remaining of mAPP mRNA | | | | | |
|---|---|---|---|---|---|---|
| animal No. | PBS | DSR-0103199 | DSR-0103216 | DSR-0103299 | DSR-0103300 | DSR-0103301 |
| 1 | 98.64 | 87.16 | 53.93 | 68.76 | 53.89 | 56.82 |
| 2 | 103.60 | 78.20 | 59.43 | 40.22 | 75.71 | 57.52 |
| 3 | 115.72 | 75.22 | 59.11 | 62.40 | 58.59 | 58.58 |
| 4 | 106.25 | 76.82 | 48.27 | 47.05 | 50.93 | 57.52 |
| 5 | 75.80 | 85.23 | 47.40 | 51.04 | 69.98 | 75.92 |
| Mean | 100.00 | 80.53 | 53.63 | 53.89 | 61.82 | 61.27 |

Table 85. shows 0% mouse APP mRNA remaining relative to PBS control in the sciatic nerve 7 days post dosing. N=5. N.D.: Not determined.

| | % remaining of mAPP mRNA | | | | | |
|---|---|---|---|---|---|---|
| animal No. | PBS | DSR-0103199 | DSR-0103216 | DSR-0103299 | DSR-0103300 | DSR-0103301 |
| 1 | 83.92 | 130.05 | 106.24 | 95.43 | 100.69 | 106.78 |
| 2 | 109.97 | 106.48 | 112.25 | 92.31 | 106.98 | 93.03 |
| 3 | 99.66 | 86.13 | 94.10 | 105.63 | 95.61 | 121.80 |
| 4 | 123.80 | 114.09 | 88.33 | 109.07 | 102.57 | 123.55 |
| 5 | 82.65 | 112.82 | 89.68 | 95.35 | 107.03 | 130.62 |
| Mean | 100.00 | 109.91 | 98.12 | 99.56 | 102.57 | 115.16 |

Table 86. shows 00 mouse APP mRNA remaining relative to PBS control in the kidney 28 days post dosing. N=5. N.D.: Not determined.

| | % remaining of mAPP mRNA | | | | | |
|---|---|---|---|---|---|---|
| animal No. | PBS | DSR-0103199 | DSR-0103216 | DSR-0103299 | DSR-0103300 | DSR-0103301 |
| 1 | 142.83 | 105.21 | 61.95 | 48.40 | 53.98 | 55.48 |
| 2 | 89.54 | 108.78 | 61.85 | 41.58 | 58.40 | 58.34 |
| 3 | 115.84 | 110.45 | 54.66 | 46.97 | 51.19 | 47.22 |
| 4 | 81.76 | 59.46 | 60.62 | 47.92 | 71.17 | 45.28 |
| 5 | 70.04 | 66.05 | 64.74 | 72.52 | 53.80 | 44.73 |
| Mean | 100.00 | 89.99 | 60.76 | 51.48 | 57.71 | 50.21 |

Table 87. shows 00 mouse APP mRNA remaining relative to PBS control in the white adipose tissue 28 days post dosing. N=5. N.D.: Not determined.

| | % remaining of mAPP mRNA | | | | | |
|---|---|---|---|---|---|---|
| animal No. | PBS | DSR-0103199 | DSR-0103216 | DSR-0103299 | DSR-0103300 | DSR-0103301 |
| 1 | 105.68 | 43.35 | 64.44 | 52.30 | 23.07 | 18.15 |
| 2 | 106.98 | 54.95 | 50.36 | 35.22 | 30.17 | 28.18 |
| 3 | 75.05 | 51.67 | 38.09 | 47.91 | 34.75 | 25.35 |
| 4 | 102.13 | 41.01 | 59.99 | 40.19 | 36.47 | 23.06 |
| 5 | 110.15 | 47.19 | 35.53 | 40.97 | 31.37 | 18.93 |
| Mean | 100.00 | 47.63 | 49.68 | 43.32 | 31.17 | 22.73 |

Table 88. shows 00 mouse APP mRNA remaining relative to PBS control in the lung 28 days post dosing. N=5. N.D.: Not determined.

| | % remaining of mAPP mRNA | | | | | |
|---|---|---|---|---|---|---|
| animal No. | PBS | DSR-0103199 | DSR-0103216 | DSR-0103299 | DSR-0103300 | DSR-0103301 |
| 1 | 87.37 | 60.12 | 80.64 | 83.06 | 88.84 | 86.21 |
| 2 | 95.25 | 54.70 | 50.24 | 57.44 | 98.53 | 76.04 |
| 3 | 133.07 | 85.99 | 86.28 | 80.34 | 115.05 | 120.09 |
| 4 | 108.00 | 99.91 | 80.82 | 93.62 | 106.36 | 108.49 |
| 5 | 76.30 | 103.14 | 72.38 | 102.19 | 104.54 | 118.93 |
| Mean | 100.00 | 80.77 | 74.07 | 83.33 | 102.66 | 101.95 |

Table 89. shows 0 mouse APP mRNA remaining relative to PBS control in the sciatic nerve 28 days post dosing. N=5. N.D.: Not determined.

| | % remaining of mAPP mRNA | | | | | |
|---|---|---|---|---|---|---|
| animal No. | PBS | DSR-0103199 | DSR-0103216 | DSR-0103299 | DSR-0103300 | DSR-0103301 |
| 1 | 97.55 | 109.13 | 92.94 | 89.25 | 115.20 | 120.54 |
| 2 | 94.03 | 133.87 | 101.66 | 97.61 | 56.72 | 130.48 |
| 3 | 84.73 | 117.17 | 96.62 | 115.81 | 117.36 | 123.16 |
| 4 | 113.86 | 90.40 | 78.62 | 117.32 | 114.89 | 108.30 |
| 5 | 109.83 | 164.38 | 72.38 | 126.63 | 142.37 | 134.97 |
| Mean | 100.00 | 122.99 | 88.44 | 109.32 | 109.31 | 123.49 |

Table 90. shows 00 mouse APP mRNA remaining relative to PBS control in the liver 28 days post dosing. N=5. N.D.: Not determined.

| | % remaining of mAPP mRNA | | | | | |
|---|---|---|---|---|---|---|
| animal No. | PBS | DSR-0103199 | DSR-0103216 | DSR-0103299 | DSR-0103300 | DSR-0103301 |
| 1 | 120.64 | 29.60 | 37.96 | 37.93 | 70.51 | 28.43 |
| 2 | 110.00 | 34.08 | 30.67 | 62.85 | 34.50 | 25.48 |
| 3 | 97.39 | 38.12 | 53.76 | 70.08 | 24.32 | 33.28 |
| 4 | 97.99 | 25.30 | 29.45 | 18.96 | 24.34 | 42.90 |
| 5 | 73.97 | 27.57 | 43.56 | 21.19 | 39.88 | 26.65 |
| Mean | 100.00 | 30.94 | 39.08 | 42.20 | 38.71 | 31.35 |

Table 91. shows 00 mouse APP mRNA remaining relative to PBS control in the quad 28 days post dosing. N=5. N.D.: Not determined.

| | % remaining of mAPP mRNA | | | | | |
|---|---|---|---|---|---|---|
| animal No. | PBS | DSR-0103199 | DSR-0103216 | DSR-0103299 | DSR-0103300 | DSR-0103301 |
| 1 | 95.16 | 66.82 | 57.30 | 55.46 | 68.68 | 67.90 |
| 2 | 120.59 | 92.11 | 53.32 | 32.47 | 60.84 | 61.95 |
| 3 | 74.23 | 59.65 | 57.08 | 73.37 | 70.74 | 67.48 |
| 4 | 99.66 | 82.46 | 50.96 | 51.96 | 68.29 | 71.28 |
| 5 | 110.35 | 87.01 | 45.35 | 49.89 | 67.97 | 66.58 |
| Mean | 100.00 | 77.61 | 52.80 | 52.63 | 67.31 | 67.04 |

Table 92. shows % mouse APP mRNA remaining relative to PBS control in the heart 28 days post dosing. N=5. N.D.: Not determined.

| | % remaining of mAPP mRNA | | | | | |
|---|---|---|---|---|---|---|
| animal No. | PBS | DSR-0103199 | DSR-0103216 | DSR-0103299 | DSR-0103300 | DSR-0103301 |
| 1 | 95.78 | 93.83 | 69.95 | 104.65 | 93.97 | 96.06 |
| 2 | 81.11 | 108.10 | 80.18 | 79.32 | 94.20 | 89.34 |
| 3 | 113.00 | 89.21 | 78.66 | 93.32 | 92.11 | 85.11 |
| 4 | 103.33 | 113.18 | 75.98 | 101.30 | 87.40 | 84.47 |
| 5 | 106.77 | 102.96 | 65.41 | 99.61 | 98.10 | 107.25 |
| Mean | 100.00 | 101.46 | 74.04 | 95.64 | 93.16 | 92.45 |

Table 93. shows % mouse APP mRNA remaining relative to PBS control in the brown adipose tissue 28 days post dosing. N=5. N.D.: Not determined.

| | % remaining of mAPP mRNA | | | | | |
|---|---|---|---|---|---|---|
| animal No. | PBS | DSR-0103199 | DSR-0103216 | DSR-0103299 | DSR-0103300 | DSR-0103301 |
| 1 | 90.51 | 68.67 | 44.32 | 39.35 | 63.71 | 71.65 |
| 2 | 72.38 | 53.59 | 55.12 | 29.34 | 53.43 | 54.67 |
| 3 | 106.20 | 61.32 | 43.70 | 29.84 | 70.51 | 57.92 |
| 4 | 141.92 | 49.80 | 38.68 | 33.32 | 0.00 | 42.33 |
| 5 | 89.00 | 39.05 | 50.65 | 45.76 | 61.68 | 77.61 |
| Mean | 100.00 | 54.49 | 46.50 | 35.52 | 49.86 | 60.84 |

Table 94. shows % mouse APP mRNA remaining relative to PBS control in the diaphragm 28 days post dosing. N=5. N.D.: Not determined.

| | % remaining of mAPP mRNA | | | | | |
|---|---|---|---|---|---|---|
| animal No. | PBS | DSR-0103199 | DSR-0103216 | DSR-0103299 | DSR-0103300 | DSR-0103301 |
| 1 | 93.20 | 90.54 | 50.00 | 65.63 | 47.80 | 95.23 |
| 2 | 107.68 | 82.19 | 62.21 | 40.27 | 79.21 | 89.17 |
| 3 | 107.34 | 126.09 | 67.31 | 60.95 | 79.69 | 88.75 |
| 4 | 121.54 | 89.87 | 57.14 | 61.48 | 85.73 | 88.29 |
| 5 | 70.24 | 97.75 | 71.41 | 62.35 | 68.53 | 89.27 |
| Mean | 100.00 | 97.29 | 61.61 | 58.13 | 72.19 | 90.14 |

Table 95. shows 00 mouse APP mRNA remaining relative to PBS control in the spleen 28 days post dosing. N=5. N.D.: Not determined. PGP-522j12

| | % remaining of mAPP mRNA | | | | | |
|---|---|---|---|---|---|---|
| animal No. | PBS | DSR-0103199 | DSR-0103216 | DSR-0103299 | DSR-0103300 | DSR-0103301 |
| 1 | 99.75 | 115.40 | 101.33 | 150.07 | 137.19 | 139.26 |
| 2 | 84.89 | 114.64 | 104.16 | 145.16 | 111.57 | 155.64 |
| 3 | 117.16 | 123.92 | 102.77 | 102.47 | 131.90 | 117.07 |
| 4 | 99.68 | 153.03 | 108.68 | 143.43 | 125.83 | 151.01 |
| 5 | 98.51 | 128.75 | 111.11 | 149.40 | 139.82 | 107.25 |
| Mean | 100.00 | 127.15 | 105.61 | 138.11 | 129.26 | 134.05 |

Table 96. shows serum ALT relative to PBS control in the Black/6 mice on 7 days and 28 days post dosing. N=5. N.D.: Not determined.

| | animal | ALT (U/L) | | | | | |
|---|---|---|---|---|---|---|---|
| | No. | PBS | DSR-0103301 | DSR-0103300 | DSR-0103299 | DSR-0103216 | DSR-0103199 |
| 7 days | 1 | 23.00 | 17.00 | 20.00 | 19.00 | 18.00 | 20.00 |
| post | 2 | 23.00 | 16.00 | 33.00 | 23.00 | 23.00 | 19.00 |
| dosing | 3 | 23.00 | 21.00 | 21.00 | 34.00 | 23.00 | 23.00 |
| | 4 | 17.00 | 18.00 | 24.00 | 84.00 | 20.00 | 23.00 |
| | 5 | 23.00 | 26.00 | 92.00 | 27.00 | 24.00 | 20.00 |
| | Mean | 21.80 | 19.60 | 38.00 | 37.40 | 21.60 | 21.00 |
| 28 days | 1 | 20.00 | 17.00 | 19.00 | 27.00 | 26.00 | N.D |
| post | 2 | 20.00 | 31.00 | 33.00 | 18.00 | 41.00 | 19.00 |
| dosing | 3 | 17.00 | 21.00 | 19.00 | 19.00 | 21.00 | 19.00 |
| | 4 | N.D | 16.00 | 21.00 | 21.00 | 22.00 | 27.00 |
| | 5 | 25.00 | 21.00 | 19.00 | 22.00 | 23.00 | 22.00 |
| | Mean | 16.40 | 21.20 | 22.20 | 21.40 | 26.60 | 17.40 |

Table 97. shows serum AST relative to PBS control in the Black/6 mice on 7 days and 28 days post dosing. N=5. N.D.: Not determined.

| | animal | AST (U/L) | | | | | |
|---|---|---|---|---|---|---|---|
| | No. | PBS | DSR-0103301 | DSR-0103300 | DSR-0103299 | DSR-0103216 | DSR-0103199 |
| 7 days | 1 | 49.00 | 41.00 | 61.00 | 72.00 | 107.00 | 119.00 |
| post | 2 | 70.00 | 39.00 | 65.00 | 62.00 | 88.00 | 62.00 |
| dosing | 3 | 51.00 | 60.00 | 91.00 | 206.00 | 111.00 | 90.00 |
| | 4 | 80.00 | 59.00 | 59.00 | 279.00 | 61.00 | 113.00 |
| | 5 | 132.00 | 50.00 | 201.00 | 87.00 | 146.00 | 82.00 |
| | Mean | 76.40 | 49.80 | 95.40 | 141.20 | 102.60 | 93.20 |
| 28 days | 1 | 127.00 | 42.00 | 44.00 | 65.00 | 161.00 | N.D |
| post | 2 | 57.00 | 59.00 | 79.00 | 48.00 | 84.00 | 58.00 |
| dosing | 3 | 57.00 | 108.00 | 86.00 | 56.00 | 61.00 | 46.00 |
| | 4 | ND | 56.00 | 77.00 | 62.00 | 55.00 | 86.00 |
| | 5 | 113.00 | 70.00 | 88.00 | 45.00 | 69.00 | 80.00 |
| | Mean | 70.80 | 67.00 | 74.80 | 55.20 | 86.00 | 54.00 |

Table 98. shows serum ALP relative to PBS control in the Black/6 mice on 7 days and 28 days post dosing. N=5. N.D.: Not determined.

| | animal | ALP (U/L) | | | | | |
|---|---|---|---|---|---|---|---|
| | No. | PBS | DSR-0103301 | DSR-0103300 | DSR-0103299 | DSR-0103216 | DSR-0103199 |
| 7 days | 1 | N.D | 129.00 | 125.00 | 103.00 | 170.00 | 105.00 |
| post | 2 | 138.00 | 126.00 | 99.00 | N.D | 152.00 | N.D |
| dosing | 3 | N.D | N.D | 78.00 | N.D | 138.00 | N.D |
| | 4 | 146.00 | N.D | N.D | N.D | 147.00 | N.D |
| | 5 | N.D | 118.00 | N.D | 130.00 | 130.00 | N.D |
| | Mean | 56.80 | 74.60 | 60.40 | 46.60 | 147.40 | 21.00 |
| 28 days | 1 | 96.00 | N.D | 104.00 | N.D | N.D | N.D |
| post | 2 | 116.00 | 122.00 | N.D | 112.00 | 136.00 | 127.00 |
| dosing | 3 | 142.00 | 113.00 | N.D | N.D | 112.00 | N.D |
| | 4 | N.D | 123.00 | N.D | 130.00 | N.D | N.D |
| | 5 | N.D | 119.00 | 113.00 | 136.00 | 152.00 | 127.00 |
| | Mean | 70.80 | 95.40 | 43.40 | 75.60 | 80.00 | 50.80 |

Table 99. shows serum GLDH relative to PBS control in the Black/6 mice on 7 days and 28 days post dosing. N=5. N.D.: Not determined.

| | animal | GLDH (U/L) | | | | | |
|---|---|---|---|---|---|---|---|
| | No. | PBS | DSR-0103301 | DSR-0103300 | DSR-0103299 | DSR-0103216 | DSR-0103199 |
| 7 days | 1 | 10.20 | 10.90 | 9.20 | 8.50 | 10.90 | 9.00 |
| post | 2 | 8.60 | 7.30 | 10.40 | 13.20 | 10.00 | 8.00 |
| dosing | 3 | 9.50 | 12.60 | 11.40 | 21.40 | 11.90 | 6.90 |
| | 4 | 7.90 | 6.20 | 12.30 | 18.00 | 9.10 | 7.40 |
| | 5 | 8.50 | 15.30 | 14.20 | 12.80 | 9.80 | 7.40 |
| | Mean | 8.94 | 10.46 | 11.50 | 14.78 | 10.34 | 7.74 |
| 28 days | 1 | 9.90 | 8.50 | 7.10 | 12.40 | 13.30 | N.D |
| post | 2 | 6.90 | 9.60 | 12.70 | 9.70 | 15.00 | 8.20 |
| dosing | 3 | 7.90 | 8.90 | 8.00 | 9.10 | 12.50 | 11.70 |
| | 4 | N.D | 12.30 | 8.60 | 9.20 | 10.40 | 8.20 |
| | 5 | 9.00 | 8.50 | 8.50 | 10.40 | 12.70 | 8.10 |
| | Mean | 6.74 | 9.56 | 8.98 | 10.16 | 12.78 | 7.24 |

Table 100. shows serum UREAN relative to PBS control in the Black/6 mice on 7 days and 28 days post dosing. N=5. N.D.: Not determined.

| | animal | UREAN (mg/dL) | | | | | |
|---|---|---|---|---|---|---|---|
| | No. | PBS | DSR-0103301 | DSR-0103300 | DSR-0103299 | DSR-0103216 | DSR-0103199 |
| 7 days | 1 | 24.00 | 19.00 | 24.00 | 26.00 | 22.00 | 29.00 |
| post | 2 | 26.00 | 28.00 | 23.00 | 25.00 | 27.00 | N.D |
| dosing | 3 | 25.00 | 23.00 | 29.00 | N.D | 25.00 | N.D |
| | 4 | 30.00 | 27.00 | 25.00 | N.D | 27.00 | 26.00 |
| | 5 | 33.00 | 28.00 | 26.00 | 26.00 | 26.00 | 26.00 |
| | Mean | 27.60 | 25.00 | 25.40 | 15.40 | 25.40 | 16.20 |
| 28 days | 1 | 31.00 | 29.00 | 24.00 | N.D | 25.00 | N.D |
| post | 2 | 30.00 | 27.00 | 28.00 | 31.00 | 26.00 | 28.00 |
| dosing | 3 | 26.00 | 30.00 | 26.00 | N.D | 26.00 | N.D |
| | 4 | N.D | 25.00 | 27.00 | 31.00 | 29.00 | 31.00 |
| | 5 | 33.00 | 27.00 | 27.00 | 24.00 | 24.00 | 28.00 |
| | Mean | 24.00 | 27.60 | 26.40 | 17.20 | 26.00 | 17.40 |

Table 101. shows serum CREAT relative to PBS control in the Black/6 mice on 7 days and 28 days post dosing. N=5. N.D.: Not determined.

| | animal | CREAT (mg/dL) | | | | | |
|---|---|---|---|---|---|---|---|
| | No. | PBS | DSR-0103301 | DSR-0103300 | DSR-0103299 | DSR-0103216 | DSR-0103199 |
| 7 days | 1 | 0.30 | 0.30 | 0.20 | 0.30 | 0.30 | 0.30 |
| post | 2 | 0.30 | 0.20 | 0.20 | 0.30 | 0.30 | 0.50 |
| dosing | 3 | 0.30 | 0.30 | 0.30 | 0.30 | 0.30 | 0.30 |
| | 4 | 0.40 | 0.40 | 0.30 | 0.30 | 0.30 | 0.40 |
| | 5 | 0.40 | 0.30 | 0.40 | 0.30 | 0.30 | 0.30 |
| | Mean | 0.34 | 0.30 | 0.28 | 0.30 | 0.30 | 0.36 |
| 28 days | 1 | 0.30 | 0.20 | 0.30 | 0.20 | 0.30 | N.D |
| post | 2 | 0.30 | 0.20 | 0.30 | 0.30 | 0.30 | 0.30 |
| dosing | 3 | 0.30 | 0.30 | 0.30 | 0.30 | 0.30 | 0.30 |
| | 4 | N.D | 0.20 | 0.30 | 0.40 | 0.30 | 0.40 |
| | 5 | 0.40 | 0.30 | 0.30 | 0.30 | 0.30 | 0.30 |
| | Mean | 0.26 | 0.24 | 0.30 | 0.30 | 0.30 | 0.26 |

Table 102. shows serum TBIL relative to PBS control in the Black/6 mice on 7 days and 28 days post dosing. N=5. N.D.: Not determined.

| | animal | TBIL (mg/dL) | | | | | |
|---|---|---|---|---|---|---|---|
| | No. | PBS | DSR-0103301 | DSR-0103300 | DSR-0103299 | DSR-0103216 | DSR-0103199 |
| 7 days | 1 | 0.04 | 0.04 | 0.06 | 0.04 | 0.13 | 0.07 |
| post | 2 | 0.05 | 0.05 | 0.07 | 0.05 | 0.06 | N.D |
| dosing | 3 | 0.07 | 0.06 | 0.06 | N.D | 0.07 | N.D |
| | 4 | 0.01 | 0.06 | 0.09 | N.D | 0.06 | 0.06 |
| | 5 | 0.04 | 0.04 | 0.05 | 0.04 | 0.08 | 0.07 |
| | Mean | 0.04 | 0.05 | 0.07 | 0.03 | 0.08 | 0.04 |
| 28 days | 1 | 0.05 | 0.07 | 0.05 | N.D | 0.06 | N.D |
| post | 2 | 0.04 | 0.08 | 0.08 | 0.07 | 0.04 | 0.08 |
| dosing | 3 | 0.09 | 0.07 | 0.09 | N.D | 0.04 | N.D |
| | 4 | N.D | 0.18 | 0.07 | 0.05 | 0.05 | 0.04 |
| | 5 | 0.07 | 0.06 | 0.08 | 0.06 | 0.13 | 0.06 |
| | Mean | 0.05 | 0.09 | 0.07 | 0.04 | 0.06 | 0.04 |

Table 103. shows the accumulation of antisense strand in the kidney at 7 days and 28 days post dosing. N=5. N.D.: Not determined.

| | animal | Kidney Guide Strand PK (ug oligo/g tissue) | | | | | |
|---|---|---|---|---|---|---|---|
| | No. | PBS | DSR-0103199 | DSR-0103216 | DSR-0103299 | DSR-0103300 | DSR-0103301 |
| 7 days | 1 | 0.00 | 18.41 | 28.59 | 69.40 | 48.56 | 60.33 |
| post | 2 | 0.00 | 19.33 | 22.90 | 63.28 | 48.54 | 70.44 |
| dosing | 3 | 0.00 | 21.00 | 30.52 | 62.15 | 60.84 | 68.75 |
| | 4 | 0.00 | 17.13 | 16.84 | 76.81 | 51.11 | 77.24 |
| | 5 | 0.00 | 20.10 | 32.42 | 82.56 | 49.74 | 84.28 |
| | Mean | 0.00 | 19.19 | 26.26 | 70.84 | 51.76 | 72.21 |
| 28 days | 1 | 0.00 | 2.52 | 4.21 | 7.21 | 6.84 | 6.88 |
| post | 2 | 0.00 | 1.66 | 4.59 | 11.92 | 5.56 | 6.19 |
| dosing | 3 | 0.00 | 2.44 | 3.33 | 12.82 | 7.08 | 8.53 |
| | 4 | 0.00 | 2.24 | 3.73 | 9.33 | 6.03 | 12.31 |
| | 5 | 0.00 | 2.14 | 5.67 | 7.90 | 7.66 | 8.22 |
| | Mean | 0.00 | 2.20 | 4.31 | 9.84 | 6.63 | 8.43 |

Table 104. shows the accumulation of antisense strand in the liver at 7 days and 28 days post dosing. N=5. N.D.: Not determined.

| | animal | Liver Guide Strand PK (ug oligo/g tissue) | | | | | |
|---|---|---|---|---|---|---|---|
| | No. | PBS | DSR-0103199 | DSR-0103216 | DSR-0103299 | DSR-0103300 | DSR-0103301 |
| 7 days | 1 | 0.00 | 14.68 | 23.94 | 34.05 | 29.04 | 38.68 |
| post | 2 | 0.00 | 17.64 | 31.55 | 45.08 | 28.22 | 42.87 |
| dosing | 3 | 0.00 | 18.92 | 22.71 | 30.85 | 35.84 | 35.55 |
| | 4 | 0.00 | 12.85 | 23.54 | 30.60 | 29.88 | 46.55 |
| | 5 | 0.00 | 22.04 | 25.97 | 33.40 | 28.82 | 37.78 |
| | Mean | 0.00 | 17.23 | 25.54 | 34.80 | 30.36 | 40.29 |
| 28 days | 1 | 0.00 | 12.14 | 22.00 | 32.62 | 32.67 | 29.44 |
| post | 2 | 0.00 | 12.81 | 20.99 | 38.26 | 32.74 | 30.33 |
| dosing | 3 | 0.00 | 14.64 | 18.81 | 41.06 | 34.65 | 29.40 |
| | 4 | 0.00 | 11.62 | 23.46 | 45.41 | 26.00 | 38.92 |
| | 5 | 0.00 | 11.09 | 23.03 | 41.72 | 29.83 | 34.23 |
| | Mean | 0.00 | 12.46 | 21.66 | 39.81 | 31.18 | 32.46 |

Table 105. shows the accumulation of antisense strand in the spleen at 7 days and 28 days post dosing. N=5. N.D.: Not determined.

| | animal | Spleen Guide Strand PK (ug oligo/g tissue) | | | | | |
|---|---|---|---|---|---|---|---|
| | No. | PBS | DSR-0103199 | DSR-0103216 | DSR-0103299 | DSR-0103300 | DSR-0103301 |
| 7 days | 1 | 0.00 | 5.52 | 9.17 | 9.97 | 9.47 | 13.43 |
| post | 2 | 0.00 | 6.42 | 8.22 | 11.63 | 10.55 | 12.29 |
| dosing | 3 | 0.00 | 7.49 | 9.00 | 9.73 | 13.39 | 14.34 |
| | 4 | 0.00 | 5.37 | 9.36 | 10.76 | 11.35 | 14.39 |
| | 5 | 0.00 | 7.03 | 9.33 | 11.35 | 11.13 | 14.18 |
| | Mean | 0.00 | 6.37 | 9.02 | 10.69 | 11.18 | 13.73 |
| 28 days | 1 | 0.00 | 3.55 | 9.00 | 8.80 | 7.84 | 9.60 |
| post | 2 | 0.00 | 3.28 | 8.73 | 10.43 | 7.44 | 10.77 |
| dosing | 3 | 0.00 | 3.58 | 9.13 | 10.14 | 8.01 | 9.10 |
| | 4 | 0.00 | 3.18 | 10.30 | 9.43 | 7.85 | 13.05 |
| | 5 | 0.00 | 2.76 | 11.68 | 14.06 | 7.40 | N.D |
| | Mean | 0.00 | 3.27 | 9.77 | 10.57 | 7.71 | 8.51 |

Table 106. shows the accumulation of antisense strand in the brown adipose at 7 days and 28 days post dosing. N=5. N.D.: Not determined.

| | animal | Brown adipose Guide Strand PK (ug oligo/g tissue) | | | | | |
|---|---|---|---|---|---|---|---|
| | No. | PBS | DSR-0103199 | DSR-0103216 | DSR-0103299 | DSR-0103300 | DSR-0103301 |
| 7 days | 1 | 0.00 | 2.66 | 7.12 | 11.14 | 4.98 | 21.30 |
| post | 2 | 0.00 | 3.58 | 5.20 | 7.68 | 4.18 | 6.15 |
| dosing | 3 | 0.00 | 1.65 | 4.99 | 7.29 | 6.47 | 23.87 |
| | 4 | 0.00 | 2.66 | 4.65 | 13.80 | 6.43 | 12.59 |
| | 5 | 0.00 | 1.88 | 5.24 | 7.38 | 3.20 | 9.00 |
| | Mean | 0.00 | 2.48 | 5.44 | 9.46 | 5.05 | 14.58 |
| 28 days | 1 | 0.00 | 4.89 | 10.79 | 14.20 | 2.95 | 9.20 |
| post | 2 | 0.00 | 2.50 | 5.45 | 12.64 | 10.21 | 10.93 |
| dosing | 3 | 0.00 | 5.66 | 8.39 | 11.47 | 3.88 | 9.66 |
| | 4 | 0.00 | 3.56 | 9.46 | 7.86 | N.D | 10.93 |
| | 5 | 0.00 | 1.92 | 12.25 | 11.99 | 7.24 | 5.52 |
| | Mean | 0.00 | 3.71 | 9.27 | 11.63 | 4.86 | 9.25 |

Table 107. shows the accumulation of antisense strand in the diaphragm at 7 days and 28 days post dosing. N=5. N.D.: Not determined.

| | animal | Diaphragm Guide Strand PK (ug oligo/g tissue) | | | | | |
|---|---|---|---|---|---|---|---|
| | No. | PBS | DSR-0103199 | DSR-0103216 | DSR-0103299 | DSR-0103300 | DSR-0103301 |
| 7 days | 1 | 0.00 | 1.86 | 5.36 | 4.51 | 2.43 | 3.70 |
| post | 2 | 0.00 | 3.26 | 4.87 | 4.31 | 3.39 | 3.72 |
| dosing | 3 | 0.00 | 2.61 | 3.29 | 3.42 | 3.90 | 3.71 |
| | 4 | 0.00 | 2.56 | 4.14 | 4.92 | 3.16 | 3.69 |
| | 5 | 0.00 | 2.31 | 3.82 | 4.90 | 3.59 | 3.96 |
| | Mean | 0.00 | 2.52 | 4.29 | 4.41 | 3.29 | 3.75 |
| 28 days | 1 | 0.00 | 0.94 | 2.91 | 2.50 | 2.36 | 2.20 |
| post | 2 | 0.00 | 1.73 | 3.41 | 4.28 | 2.49 | 2.44 |
| dosing | 3 | 0.00 | 1.00 | 4.53 | 3.50 | 2.20 | 2.34 |
| | 4 | 0.00 | 1.11 | 3.80 | 3.34 | 2.24 | 3.44 |
| | 5 | 0.00 | 1.05 | 3.07 | 3.26 | 2.49 | 2.54 |
| | Mean | 0.00 | 1.16 | 3.54 | 3.37 | 2.36 | 2.59 |

Table 108. shows the accumulation of antisense strand in the lung at 7 days and 28 days post dosing. N=5. N.D.: Not determined.

| | animal | Lung Guide Strand PK (ug oligo/g tissue) | | | | | |
|---|---|---|---|---|---|---|---|
| | No. | PBS | DSR-0103199 | DSR-0103216 | DSR-0103299 | DSR-0103300 | DSR-0103301 |
| 7 days | 1 | 0.00 | 0.85 | 2.46 | 3.44 | 1.11 | 4.55 |
| post | 2 | 0.00 | 1.01 | 2.18 | 1.82 | 1.67 | 3.45 |
| dosing | 3 | 0.00 | 1.12 | 1.89 | 2.73 | 2.87 | 2.25 |
| | 4 | 0.00 | 0.90 | 2.39 | 4.13 | 2.19 | 3.35 |
| | 5 | 0.00 | 1.58 | 3.28 | 4.04 | 2.93 | 2.35 |
| | Mean | 0.00 | 1.09 | 2.44 | 3.23 | 2.15 | 3.19 |
| 28 days | 1 | 0.00 | 0.66 | 2.61 | 3.41 | 2.20 | 2.60 |
| post | 2 | 0.00 | 0.81 | 2.42 | 3.43 | 2.53 | 2.34 |
| dosing | 3 | 0.00 | 0.82 | 2.51 | 3.27 | 2.03 | 2.61 |
| | 4 | 0.00 | 0.61 | 3.90 | 3.75 | 1.57 | 3.53 |
| | 5 | 0.00 | 0.54 | 4.41 | 4.56 | 2.68 | 3.25 |
| | Mean | 0.00 | 0.69 | 3.17 | 3.68 | 2.20 | 2.87 |

Table 109. shows the accumulation of antisense strand in the heart at 7 days and 28 days post dosing. N=5. N.D.: Not determined.

| | animal | Heart Guide Strand PK (ug oligo/g tissue) | | | | | |
|---|---|---|---|---|---|---|---|
| | No. | PBS | DSR-0103199 | DSR-0103216 | DSR-0103299 | DSR-0103300 | DSR-0103301 |
| 7 days | 1 | 0.00 | 1.47 | 2.91 | 2.27 | 1.69 | 4.35 |
| post | 2 | 0.00 | 1.75 | 2.37 | 2.55 | 2.39 | 4.02 |
| dosing | 3 | 0.00 | 2.86 | 2.42 | 2.63 | 2.56 | 3.10 |
| | 4 | 0.00 | 1.63 | 3.94 | 2.18 | 1.57 | 3.28 |
| | 5 | 0.00 | 1.68 | 2.86 | 3.10 | 2.53 | 3.51 |
| | Mean | 0.00 | 1.88 | 2.90 | 2.55 | 2.15 | 3.65 |
| 28 days | 1 | 0.00 | 1.09 | 1.28 | 2.03 | 1.94 | 2.46 |
| post | 2 | 0.00 | 1.05 | 1.75 | 2.31 | 1.92 | 3.03 |
| dosing | 3 | 0.00 | 1.47 | 1.84 | 2.11 | 1.45 | 2.56 |
| | 4 | 0.00 | 1.34 | 2.08 | 2.23 | 1.66 | 3.56 |
| | 5 | 0.00 | 1.15 | 2.56 | 2.52 | 1.92 | 2.66 |
| | Mean | 0.00 | 1.22 | 1.90 | 2.24 | 1.78 | 2.85 |

Table 110. shows the accumulation of antisense strand in the quad at 7 days and 28 days post dosing. N=5. N.D.: Not determined.

| | animal | Quad Guide Strand PK (ug oligo/g tissue) | | | | | |
|---|---|---|---|---|---|---|---|
| | No. | PBS | DSR-0103199 | DSR-0103216 | DSR-0103299 | DSR-0103300 | DSR-0103301 |
| 7 days | 1 | 0.00 | 0.59 | 2.41 | 2.34 | 1.27 | 0.98 |
| post | 2 | 0.00 | 1.22 | 2.17 | 2.72 | 1.82 | 2.50 |
| dosing | 3 | 0.00 | 1.64 | 1.51 | 2.68 | 1.77 | 1.84 |
| | 4 | 0.00 | 1.14 | 2.16 | 1.92 | 2.00 | 1.46 |
| | 5 | 0.00 | 1.38 | 1.91 | 2.33 | 1.15 | 1.96 |
| | Mean | 0.00 | 1.19 | 2.03 | 2.40 | 1.60 | 1.75 |
| 28 days | 1 | 0.00 | 0.83 | 1.65 | 0.97 | 1.25 | 1.01 |
| post | 2 | 0.00 | 0.52 | 1.74 | 1.97 | 1.09 | 1.06 |
| dosing | 3 | 0.00 | 1.05 | 1.64 | 1.10 | 1.09 | 0.88 |
| | 4 | 0.00 | 0.70 | 1.80 | 1.77 | 1.22 | 1.34 |
| | 5 | 0.00 | 0.57 | 0.98 | 1.46 | 1.63 | 1.67 |
| | Mean | 0.00 | 0.74 | 1.56 | 1.45 | 1.26 | 1.19 |

Table 111. shows the accumulation of antisense strand in the white adipose at 7 days and 28 days post dosing. N=5. N.D.: Not determined.

| | animal | White adipose Guide Strand PK (ug oligo/g tissue) | | | | | |
|---|---|---|---|---|---|---|---|
| | No. | PBS | DSR-0103199 | DSR-0103216 | DSR-0103299 | DSR-0103300 | DSR-0103301 |
| 7 days | 1 | 0.00 | 1.09 | 1.45 | 1.49 | 0.93 | 1.11 |
| post | 2 | 0.00 | 1.46 | 0.99 | 1.33 | 0.84 | 2.52 |
| dosing | 3 | 0.00 | 1.08 | 0.96 | 1.71 | 2.17 | 1.64 |
| | 4 | 0.00 | 0.89 | 1.01 | 1.69 | 0.93 | 2.62 |
| | 5 | 0.00 | 1.05 | 0.96 | 3.26 | 1.39 | 1.79 |
| | Mean | 0.00 | 1.11 | 1.07 | 1.90 | 1.25 | 1.93 |
| 28 days | 1 | 0.00 | 0.60 | 0.81 | 1.39 | 0.49 | 0.99 |
| post | 2 | 0.00 | 0.71 | 0.71 | 1.05 | 1.00 | 2.16 |
| dosing | 3 | 0.00 | 0.72 | 0.72 | 0.66 | 0.77 | 1.15 |
| | 4 | 0.00 | 0.46 | 0.93 | 0.61 | 0.73 | 2.45 |
| | 5 | 0.00 | 0.47 | 1.01 | 1.54 | 1.37 | 2.33 |
| | Mean | 0.00 | 0.59 | 0.84 | 1.05 | 0.87 | 1.82 |

Table 112. shows Ago 2 loading of guide strand relative to mi-143 in liver. N=3

| | Ct: | Ct: | Ct: | Ct: | Relative |
|---|---|---|---|---|---|
| | mAPP/ | miR- | mAPP/ | miR- | mAPP/miR- |
| | Ago2 | 143/Ago2 | IgG | 143/IgG | 143 |
| | Day 7 | | | | |
| DSR-0103199-1 | 30.56 | 32.94 | 33.38 | 40 | 0.053 |
| DSR-0103199-2 | 30.06 | 31.57 | 33.1 | 40 | 0.024 |
| DSR-0103199-3 | 29.73 | 31.45 | 33.24 | 40 | 0.030 |
| DSR-0103216-1 | 28.16 | 29.58 | 31.82 | 40 | 0.018 |
| DSR-0103216-2 | 28.63 | 30.07 | 30.12 | 39.35 | 0.005 |
| DSR-0103216-3 | 29.18 | 31.35 | 30.32 | 39.47 | 0.006 |
| DSR-0103299-1 | 28.74 | 30.02 | 31.16 | 39.24 | 0.009 |
| DSR-0103299-2 | 27.84 | 29.62 | 31.07 | 36.47 | 0.081 |
| DSR-0103299-3 | 28.15 | 30.81 | 30.61 | 38.94 | 0.020 |
| DSR-0103300-1 | 27.42 | 31.22 | 29.5 | 39.16 | 0.017 |
| DSR-0103300-2 | 26.92 | 30.49 | 30.07 | 39.65 | 0.016 |
| DSR-0103300-3 | 26.93 | 30.7 | 29.75 | 38.87 | 0.025 |
| DSR-0103301-1 | 27.18 | 29.31 | 31.16 | 38.87 | 0.021 |
| DSR-0103301-2 | 27.15 | 28.75 | 31.4 | 38.32 | 0.025 |
| DSR-0103301-3 | 27.23 | 29.63 | 31.32 | 39.28 | 0.021 |
| | Day 28 | | | | |
| DSR-0103199-1 | 28.66 | 28.68 | 32.72 | 40 | 0.007 |
| DSR-0103199-2 | 28.61 | 28.29 | 32.96 | 40 | 0.006 |
| DSR-0103199-3 | 28.66 | 28.32 | 32.92 | 40 | 0.006 |
| DSR-0103216-1 | 27.54 | 28.45 | 30.99 | 39.53 | 0.005 |
| DSR-0103216-2 | 27.29 | 28.09 | 31.22 | 40 | 0.004 |
| DSR-0103216-3 | 27.51 | 28.33 | 31.07 | 39.76 | 0.004 |
| DSR-0103299-1 | 27.95 | 28.25 | 31.59 | 39.96 | 0.004 |
| DSR-0103299-2 | 27.91 | 28.85 | 31.79 | 40 | 0.006 |
| DSR-0103299-3 | 28.09 | 28.92 | 31.76 | 40 | 0.006 |
| DSR-0103300-1 | 27.38 | 28.92 | 31.81 | 39.32 | 0.016 |
| DSR-0103300-2 | 27.90 | 29.44 | 31.88 | 40 | 0.010 |
| DSR-0103300-3 | 28.46 | 30.86 | 31.2 | 40 | 0.012 |
| DSR-0103301-1 | 27.96 | 29.25 | 31.52 | 40 | 0.007 |
| DSR-0103301-2 | 27.68 | 29.32 | 31.67 | 40 | 0.010 |
| DSR-0103301-3 | 28.16 | 29.41 | 31.66 | 40 | 0.007 |

Table 113. shows Ago 2 loading of guide strand relative to miR-143 in kidney. N=3

| | Ct: | Ct: | Ct: | Ct: | Relative |
|---|---|---|---|---|---|
| | mAPP/ | miR- | mAPP/ | miR- | mAPP/miR- |
| | Ago2 | 143/Ago2 | IgG | 143/IgG | 143 |
| | Day 7 | | | | |
| DSR-0103199-1 | 28.37 | 31.75 | 29.03 | 34.13 | 0.304 |
| DSR-0103199-2 | 26.74 | 31.26 | 29.22 | 34.03 | 0.818 |
| DSR-0103199-3 | 27.29 | 31.43 | 28.57 | 33.6 | 0.540 |
| DSR-0103216-1 | 27.21 | 31.54 | 29.21 | 34.08 | 0.688 |
| DSR-0103216-2 | 27.90 | 31.49 | 29.11 | 33.41 | 0.611 |
| DSR-0103216-3 | 28.19 | 31.76 | 29.72 | 33.49 | 0.871 |
| DSR-0103299-1 | 28.46 | 31.56 | 28.9 | 32.38 | 0.768 |
| DSR-0103299-2 | 29.44 | 32.15 | 29.6 | 30.25 | 4.170 |
| DSR-0103299-3 | 29.16 | 31.4 | 29.93 | 33.62 | 0.366 |
| DSR-0103300-1 | 30.26 | 31.82 | 30.51 | 32.62 | 0.683 |
| DSR-0103300-2 | 30.47 | 31.67 | 30.77 | 33.84 | 0.274 |
| DSR-0103300-3 | 30.38 | 31.89 | 30.65 | 33.99 | 0.281 |
| DSR-0103301-1 | 20.57 | 31.75 | 19.45 | 32.26 | 0.323 |
| DSR-0103301-2 | 21.10 | 31.71 | 19.64 | 32.5 | 0.210 |
| DSR-0103301-3 | 21.12 | 31.68 | 20.9 | 32.93 | 0.361 |
| | Day 28 | | | | |
| DSR-0103199-1 | 31.33 | 33.46 | 31.05 | 34.73 | 0.342 |
| DSR-0103199-2 | 31.15 | 31.63 | 30.52 | 33.73 | 0.151 |
| DSR-0103199-3 | 31.21 | 33.54 | 30.67 | 36.01 | 0.124 |
| DSR-0103216-1 | 30.81 | 32.08 | 30.11 | 33 | 0.325 |
| DSR-0103216-2 | 30.79 | 32.5 | 30.26 | 33.28 | 0.403 |
| DSR-0103216-3 | 30.73 | 32.3 | 30.32 | 33.82 | 0.262 |
| DSR-0103299-1 | 30.73 | 31.9 | 30.52 | 33.06 | 0.387 |
| DSR-0103299-2 | 30.69 | 31.86 | 30.34 | 33.68 | 0.222 |
| DSR-0103299-3 | 30.30 | 31.26 | 30.58 | 34.16 | 0.163 |
| DSR-0103300-1 | 30.37 | 30.6 | 30.41 | 33.48 | 0.140 |
| DSR-0103300-2 | 30.41 | 31.69 | 30.64 | 33.32 | 0.379 |
| DSR-0103300-3 | 30.68 | 32.63 | 30.4 | 35.03 | 0.156 |
| DSR-0103301-1 | 27.45 | 33.29 | 26.84 | 34.67 | 0.252 |
| DSR-0103301-2 | 27.47 | 31.64 | 25.9 | 33.48 | 0.094 |
| DSR-0103301-3 | 25.69 | 32.27 | 24.7 | 33.04 | 0.295 |

Table 114. shows Ago 2 loading of guide strand relative to mi-143 in white adipose tissue. N=3

| | Ct: | Ct: | Ct: | Ct: | Relative |
|---|---|---|---|---|---|
| | mAPP/ | miR- | mAPP/ | miR- | mAPP/miR- |
| | Ago2 | 143/Ago2 | IgG | 143/IgG | 143 |
| | Day 7 | | | | |
| DSR-0103199-1 | 30.25 | 24.04 | 30.11 | 32.59 | 0.002 |
| DSR-0103199-2 | 30.18 | 24.18 | 30.7 | 32.59 | 0.004 |
| DSR-0103199-3 | 30.11 | 22.66 | 30.97 | 32.77 | 0.002 |
| DSR-0103216-1 | 30.08 | 23.41 | 30.6 | 32.83 | 0.002 |
| DSR-0103216-2 | 30.36 | 23.41 | 31.01 | 33.39 | 0.002 |
| DSR-0103216-3 | 30.60 | 23.23 | 31.03 | 33.61 | 0.001 |
| DSR-0103299-1 | 30.42 | 24.24 | 31.06 | 33.82 | 0.002 |
| DSR-0103299-2 | 30.49 | 23.29 | 30.88 | 32.18 | 0.003 |
| DSR-0103299-3 | 30.41 | 23.46 | 31.18 | 34.16 | 0.001 |
| DSR-0103300-1 | 30.46 | 24.4 | 31.04 | 33.78 | 0.002 |
| DSR-0103300-2 | 30.56 | 24.05 | 31.35 | 34.85 | 0.001 |
| DSR-0103300-3 | 30.51 | 24.58 | 31.28 | 34.4 | 0.002 |
| DSR-0103301-1 | 25.50 | 23.23 | 26.5 | 31.54 | 0.006 |
| DSR-0103301-2 | 25.60 | 23.07 | 26.86 | 31.64 | 0.006 |
| DSR-0103301-3 | 24.64 | 22.27 | 25.85 | 31.52 | 0.004 |
| | Day 28 | | | | |
| DSR-0103199-1 | 29.23 | 23.17 | 29.17 | 31.1 | 0.004 |
| DSR-0103199-2 | 30.78 | 24.38 | 30.93 | 31.56 | 0.008 |
| DSR-0103199-3 | 30.16 | 23.29 | 30.12 | 31.03 | 0.005 |
| DSR-0103216-1 | 30.78 | 22.74 | 30.4 | 31.35 | 0.002 |
| DSR-0103216-2 | 30.78 | 22.56 | 30.47 | 31.04 | 0.002 |
| DSR-0103216-3 | 30.62 | 23.99 | 30.82 | 31.42 | 0.007 |
| DSR-0103299-1 | 30.64 | 23.6 | 31.24 | 33.3 | 0.002 |
| DSR-0103299-2 | 30.58 | 23.46 | 31.05 | 32.04 | 0.004 |
| DSR-0103299-3 | 30.43 | 23.4 | 31.27 | 33.13 | 0.002 |
| DSR-0103300-1 | 30.66 | 24.48 | 31.33 | 34.57 | 0.001 |
| DSR-0103300-2 | 30.25 | 24.24 | 32.26 | 37.54 | 0.000 |
| DSR-0103300-3 | 30.45 | 25.01 | 31.2 | 33.97 | 0.003 |
| DSR-0103301-1 | 26.03 | 21.96 | 26.95 | 31.16 | 0.003 |
| DSR-0103301-2 | 25.47 | 22.76 | 26.71 | 31.96 | 0.004 |
| DSR-0103301-3 | 26.71 | 23.73 | 26.58 | 32.18 | 0.003 |

### Example 13. Provided Oligonucleotides and Compositions are Active In Vivo

All animal procedures were performed under IACUC guidelines. Male 8-9 weeks of age C57BL/6 mice underwent surgery for the implantation of intracerebroventricular cannulas. At 10-11 weeks of age the same mice were dosed 100 μg on Day 0 by intracerebroventricular injection. After 8 weeks animals in groups 1-5 were euthanized by CO₂ asphyxiation followed by thoracotomy. After cardiac perfusion with PBS brain samples were taken from both hemispheres (cortex, striatum, hippocampus, cerebellum, and brain stem) and flash frozen on dry ice. After 12 weeks animals in groups 6-10 were euthanized by CO₂ asphyxiation followed by thoracotomy and terminal blood collection. After cardiac perfusion with PBS brain samples were taken from both hemispheres (cortex, striatum, hippocampus, cerebellum, brain stem, and spinal cord) and flash frozen on dry ice. After 16 weeks animals in groups 11-15 were euthanized by CO₂ asphyxiation followed by thoracotomy and terminal blood collection. After cardiac perfusion with PBS brain samples were taken from both hemispheres (cortex, striatum, hippocampus, cerebellum, brain stem, and spinal cord) and flash frozen on dry ice. One animals from each group had the right hemisphere of their brain placed in buffered 10% formalin. Total RNA from different CNS regions was extracted using RNeasy 96 kit (Qiagen), after tissue lysis with TRIzol and chloroform. cDNA production from RNA samples were performed using High-Capacity cDNA Reverse Transcription kit (Thermo Fisher) following manufacturer's instructions and qPCR analysis performed in CFX System using iQ Multiplex Powermix (Bio-Rad). For mouse APP mRNA, the following qPCR assay were utilized: Thermofisher Taqman qPCR assay ID Mm01344172-ml. Mouse HPRT was used as normalizer (Forward 5′CAAACTTTGCTTTCCCTGGTT3′, Reverse 5′TGGCCTGTATCCAACACTTC3′, Probe 5′/5HEX/ACCAGCAAG/Zen/CTTGCAACCTTAACC/3IABkFQ/3′. Thermofisher Taqman qPCR assay ID for Calb1: Mm00486647_m1, Grid2: Mm00515047_m1, Pcp2: Mm00435514_m1, Gfap: Mm01253033_m1, Aif1: Mm00479862-g1. Thermofisher Taqman qPCR assay ID Mouse Tubb3 which was used as normalizer: Mm00727586-s1. Oligonucleotide accumulation in CNS regions was determined by sandwich ELISA.

To evaluate the safety of siRNAs used, serum NfL levels were measured at 4-, 6-, and 8-weeks following administration. Monoclonal antibody UD1 (Uman Diagnotics) was covalently coupled to MagPlex-C Microspheres #12 (MC10012-01, Luminex) using xMAP Antibody Coupling Kit (#40-50016, Luminex). Serum samples from mice were diluted as 1:4 with Specimen Diluent Buffer (Quidel #A3670) and incubated with UD1 coupled microspheres (2500 beads/well) in a 96-well plate for 18-20 hours at 4 C on an orbital plated shaker. Following three washes with Wash Buffer (EMD Millipore #L-WB), the wells were subjected to an incubation step with 25 μL of Detection antibody (1:500, UD3, Uman Diagnostics) on a plate shaker for 2 hours at ambient temperature. 25 μL of Streptavidin-Phycoerthrin (EMD Millipore #LB-SAPE10) was introduced into each well, followed by an incubation period of 30 minutes with continuous agitation. Following three rounds of washing, 150 microliters of Sheath Fluid PLUS (EMD Millipore #40-50021) was introduced, and subsequently, the beads were resuspended on a plate shaker for a duration of 5 minutes. The fluorescence intensity signal of the beads was measured using FLEXMAP 3D (Luminex) instrument with xPONENT software (Luminex). The analyses were conducted in duplicate to ensure accuracy and reliability. The standard NfL protein (Progen #RPC20469) was used to prepare five standards at the concentration from 30 to 200,000 μg/ml in Assay buffer (EMD Millipore #L-AB) Unspiked assay buffer was used as blank. Standard curves were fitted using 5-Parameter Logistic regression analysis by XPONENT software. Average NfL concentration of each group was calculated and plotted using GraphPad Prism. Minimal changes in the serum levels of NfL were observed in the mice treated with siRNA, in comparison to the group treated with PBS. There was no statistically significant difference observed between the groups.

Cerebellum total RNA extracted from mice treated with PBS, DSR-0103432, DSR-0103299, DSR-0103300, and DSR-0103301 (3 animals from each group) were RNA-sequenced and Fastq's were obtained from Novogene. nf-core/rnaseq (version 3.10.1) pipeline was used to process the data, with the specific packages following. Adapters were trimmed using Trim Galore!(Version 0.6.7 & cutadapt version 3.4) then aligned using STAR (version 2.7.9a) and transcript counts quantified using Salmon (version 1.9.0). Aligned bam files were processed using R (version 4.3.0) and differential expression results calculated using DEseq2 (version 1.40.2) and plotted using EnhancedVolcano (version 1.18.0) with cutoffs greater than |log 2foldChange|>1 and padj (Benjamini-Hochberg) less than 0.01.

Table 115. shows % mouse APP mRNA remaining relative to PBS control in the cerebellum 8 weeks post dosing. N=6. N.D. Not determined.

| | % remaining of mAPP mRNA | | | | |
|---|---|---|---|---|---|
| | | DSR- | DSR- | DSR- | DSR- |
| animal No. | PBS | 0103432 | 0103299 | 0103300 | 0103301 |
| 1 | 97.70 | 136.10 | 36.10 | 32.00 | 21.10 |
| 2 | 91.50 | 140.50 | 23.60 | 86.90 | 24.20 |
| 3 | 115.60 | 113.50 | 25.90 | 19.30 | 15.40 |
| 4 | 99.50 | 102.90 | 31.30 | 42.50 | 24.30 |
| 5 | 90.10 | 115.40 | 23.40 | 19.90 | 17.80 |
| 6 | 105.50 | 81.00 | 27.20 | 20.30 | 17.30 |
| Mean | 99.98 | 114.90 | 27.92 | 36.82 | 20.02 |

Table 116. shows 0% mouse APP mRNA remaining relative to PBS control in the spinal cord 8 weeks post dosing. N=6. N.D.: Not determined.

| | % remaining of mAPP mRNA | | | | |
|---|---|---|---|---|---|
| | | DSR- | DSR- | DSR- | DSR- |
| animal No. | PBS | 0103432 | 0103299 | 0103300 | 0103301 |
| 1 | 94.30 | 109.80 | 26.90 | 12.70 | 7.60 |
| 2 | 97.70 | 99.20 | 16.60 | 76.30 | 8.10 |
| 3 | 103.30 | 119.30 | 18.90 | 15.40 | 8.60 |
| 4 | 88.80 | 110.20 | 26.20 | 29.10 | 9.60 |
| 5 | 106.60 | 148.50 | 20.90 | 9.40 | 8.30 |
| 6 | 109.30 | 115.30 | 24.60 | 14.60 | 5.50 |
| Mean | 100.00 | 117.05 | 22.35 | 26.25 | 7.95 |

Table 117. shows 00 mouse APP mRNA remaining relative to PBS control in the cortex 8 weeks post dosing. N=6. N.D.: Not determined.

| | % remaining of mAPP mRNA | | | | |
|---|---|---|---|---|---|
| | | DSR- | DSR- | DSR- | DSR- |
| animal No. | PBS | 0103432 | 0103299 | 0103300 | 0103301 |
| 1 | 98.30 | 92.30 | 36.90 | 22.00 | 17.00 |
| 2 | 99.00 | 100.60 | 35.70 | 32.10 | 28.80 |
| 3 | 93.80 | 110.10 | 21.30 | 23.20 | 21.50 |
| 4 | 102.00 | 97.50 | 30.50 | 54.80 | 22.00 |
| 5 | 96.50 | 119.70 | 36.60 | 17.90 | 7.90 |
| 6 | 110.50 | 116.20 | 18.60 | 18.70 | 10.00 |
| Mean | 100.02 | 106.07 | 29.93 | 28.12 | 17.87 |

Table 118. shows 0% mouse APP mRNA remaining relative to PBS control in the hippocampus 8 weeks post dosing. N=6. N.D.: Not determined.

| | % remaining of mAPP mRNA | | | | |
|---|---|---|---|---|---|
| | | DSR- | DSR- | DSR- | DSR- |
| animal No. | PBS | 0103432 | 0103299 | 0103300 | 0103301 |
| 1 | 93.10 | 105.20 | 20.70 | 10.10 | 7.40 |
| 2 | 103.50 | 98.90 | 16.00 | 15.30 | 11.60 |
| 3 | 112.20 | 113.50 | 10.00 | 9.70 | 9.30 |
| 4 | 94.30 | 116.50 | 18.00 | 30.80 | 13.30 |
| 5 | 92.30 | 107.90 | 11.90 | 10.20 | 5.90 |
| 6 | 104.60 | 107.90 | 10.90 | 8.80 | 8.60 |
| Mean | 100.00 | 108.32 | 14.58 | 14.15 | 9.35 |

Table 119. shows 00 mouse APP mRNA remaining relative to PBS control in the striatum 8 weeks post dosing. N=6. N.D.: Not determined.

| | % remaining of mAPP mRNA | | | | |
|---|---|---|---|---|---|
| | | DSR- | DSR- | DSR- | DSR- |
| animal No. | PBS | 0103432 | 0103299 | 0103300 | 0103301 |
| 1 | 105.20 | 116.30 | 27.70 | 24.40 | 22.60 |
| 2 | 107.90 | 85.40 | 42.60 | 28.50 | 45.60 |
| 3 | 95.10 | 97.00 | 20.10 | 18.30 | 15.50 |
| 4 | 97.80 | 97.40 | 23.20 | 51.70 | 24.30 |
| 5 | 95.90 | 95.50 | 26.70 | 10.60 | 13.70 |
| 6 | 98.00 | 94.70 | 25.50 | 12.30 | 13.60 |
| Mean | 99.98 | 97.72 | 27.63 | 24.30 | 22.55 |

Table 120. shows 0% mouse APP mRNA remaining relative to PBS control in the brainstem 8 weeks post dosing. N=6. N.D.: Not determined.

| | % remaining of mAPP mRNA | | | | |
|---|---|---|---|---|---|
| | | DSR- | DSR- | DSR- | DSR- |
| animal No. | PBS | 0103432 | 0103299 | 0103300 | 0103301 |
| 1 | 103.90 | 126.80 | 33.40 | 16.80 | 13.30 |
| 2 | 117.40 | 109.70 | 22.60 | 59.00 | 13.70 |
| 3 | 99.50 | 108.80 | 15.80 | 17.50 | 10.60 |
| 4 | 92.00 | 101.60 | 20.20 | 42.60 | 13.80 |
| 5 | 94.30 | 98.00 | 16.40 | 13.20 | 10.00 |
| 6 | 92.90 | 101.10 | 20.60 | 12.30 | 8.80 |
| Mean | 100.00 | 107.67 | 21.50 | 26.90 | 11.70 |

Table 121. shows 00 mouse APP mRNA remaining relative to PBS control in the brainstem 12 weeks post dosing. N=6. N.D.: Not determined.

| | % remaining of mAPP mRNA | | | | |
|---|---|---|---|---|---|
| | | DSR- | DSR- | DSR- | DSR- |
| animal No. | PBS | 0103432 | 0103299 | 0103300 | 0103301 |
| 1 | 110.00 | 103.40 | 21.00 | 17.70 | 18.00 |
| 2 | 104.20 | 99.10 | 31.60 | 12.00 | 10.40 |
| 3 | 101.00 | 101.80 | 17.30 | 14.10 | 10.60 |
| 4 | 94.20 | 82.60 | 16.90 | 16.90 | N.D |
| 5 | 110.00 | 107.10 | 35.40 | 19.90 | 10.00 |
| 6 | 80.60 | 86.70 | 23.50 | 12.10 | 13.10 |
| Mean | 100.00 | 96.78 | 24.28 | 15.45 | 10.35 |

Table 122. shows 0% mouse APP mRNA remaining relative to PBS control in the striatum 12 weeks post dosing. N=6. N.D.: Not determined.

| | % remaining of mAPP mRNA | | | | |
|---|---|---|---|---|---|
| | | DSR- | DSR- | DSR- | DSR- |
| animal No. | PBS | 0103432 | 0103299 | 0103300 | 0103301 |
| 1 | 83.30 | 103.80 | 41.00 | 18.80 | 31.70 |
| 2 | 125.60 | 71.30 | 44.00 | 11.10 | 10.40 |
| 3 | 95.30 | 96.00 | 16.20 | 25.70 | 13.20 |
| 4 | 104.40 | 98.90 | 24.30 | 44.20 | N.D |
| 5 | 98.70 | 84.30 | 52.80 | 75.70 | 15.40 |
| 6 | 92.70 | 89.00 | 69.20 | 11.40 | 31.20 |
| Mean | 100.00 | 90.55 | 41.25 | 31.15 | 16.98 |

Table 123. shows 00 mouse APP mRNA remaining relative to PBS control in the hippocampus 12 weeks post dosing. N=6. N.D.: Not determined.

| | % remaining of mAPP mRNA | | | | |
|---|---|---|---|---|---|
| | | DSR- | DSR- | DSR- | DSR- |
| animal No. | PBS | 0103432 | 0103299 | 0103300 | 0103301 |
| 1 | 102.50 | 92.20 | 18.70 | 8.20 | 8.50 |
| 2 | 110.30 | 107.20 | 25.00 | 8.10 | 7.70 |
| 3 | 90.20 | 90.40 | 9.10 | 8.70 | 7.00 |
| 4 | 97.50 | 109.60 | 12.40 | 18.50 | N.D |
| 5 | 89.60 | 99.10 | 33.10 | 21.60 | 10.80 |
| 6 | 109.90 | 109.50 | 36.80 | 7.50 | 23.00 |
| Mean | 100.00 | 101.33 | 22.52 | 12.10 | 9.50 |

Table 124. shows 0% mouse APP mRNA remaining relative to PBS control in the cortex 12 weeks post dosing. N=6. N.D.: Not determined.

| | % remaining of mAPP mRNA | | | | |
|---|---|---|---|---|---|
| | | DSR- | DSR- | DSR- | DSR- |
| animal No. | PBS | 0103432 | 0103299 | 0103300 | 0103301 |
| 1 | 98.70 | 116.60 | 37.20 | 29.70 | 24.00 |
| 2 | 95.30 | 116.40 | 47.20 | 20.90 | 18.20 |
| 3 | 94.10 | 108.00 | 15.20 | 21.00 | 12.20 |
| 4 | 100.00 | 118.40 | 25.70 | 36.40 | N.D |
| 5 | 108.10 | 96.10 | 62.90 | 48.60 | 17.50 |
| 6 | 103.90 | 104.90 | 56.50 | 8.10 | 33.50 |
| Mean | 100.02 | 110.07 | 40.78 | 27.45 | 17.57 |

Table 125. shows 00 mouse APP mRNA remaining relative to PBS control in the spinal cord 12 weeks post dosing. N=6. N.D.: Not determined.

| | % remaining of mAPP mRNA | | | | |
|---|---|---|---|---|---|
| | | DSR- | DSR- | DSR- | DSR- |
| animal No. | PBS | 0103432 | 0103299 | 0103300 | 0103301 |
| 1 | 111.00 | 90.10 | 25.80 | 15.70 | 10.40 |
| 2 | 105.60 | 101.80 | 27.70 | 11.80 | 8.20 |
| 3 | 103.30 | 75.60 | 17.30 | 13.20 | 9.50 |
| 4 | 106.30 | 106.40 | 22.10 | 12.90 | N.D |
| 5 | 90.70 | 102.90 | 50.90 | 18.30 | 8.70 |
| 6 | 83.00 | 104.20 | 45.00 | 10.40 | 8.80 |
| Mean | 99.98 | 96.83 | 31.47 | 13.72 | 7.60 |

Table 126. shows 0% mouse APP mRNA remaining relative to PBS control in the cerebellum 12 weeks post dosing. N=6. N.D.: Not determined.

| | % remaining of mAPP mRNA | | | | |
|---|---|---|---|---|---|
| | | DSR- | DSR- | DSR- | DSR- |
| animal No. | PBS | 0103432 | 0103299 | 0103300 | 0103301 |
| 1 | 99.50 | 111.20 | 20.10 | 16.10 | 15.80 |
| 2 | 94.30 | 96.40 | 34.80 | 17.90 | 14.30 |
| 3 | 98.00 | 101.70 | 21.00 | 14.50 | 14.90 |
| 4 | 96.30 | 108.80 | 21.60 | 25.90 | N.D |
| 5 | 97.20 | 112.40 | 44.40 | 35.00 | 16.60 |
| 6 | 114.60 | 116.40 | 43.00 | 21.30 | 18.60 |
| Mean | 99.98 | 107.82 | 30.82 | 21.78 | 13.37 |

Table 126A. Shows 00 mouse APP mRNA remaining relative to PBS control in the brainstem 16 weeks post dosing. N=6. N.D.: Not determined.

| | % remaining of mAPP mRNA | | | | |
|---|---|---|---|---|---|
| | | DSR- | DSR- | DSR- | DSR- |
| animal No. | PBS | 0103432 | 0103299 | 0103300 | 0103301 |
| 1 | 106.90 | 96.90 | 32.30 | 20.20 | 14.50 |
| 2 | 88.10 | 103.30 | 29.90 | 18.50 | 13.80 |
| 3 | 99.30 | 84.60 | 27.30 | 16.30 | 36.00 |
| 4 | 95.80 | 94.50 | 38.30 | 27.30 | 13.80 |
| 5 | N.D | 106.20 | 37.30 | 19.10 | 14.60 |
| 6 | 109.80 | 100.20 | 40.00 | 17.70 | 17.50 |
| Mean | 99.98 | 97.62 | 34.18 | 19.85 | 18.37 |

Table 126B. Shows 0% mouse APP mRNA remaining relative to PBS control in the striatum 16 weeks post dosing. N=6. N.D.: Not determined.

| | % remaining of mAPP mRNA | | | | |
|---|---|---|---|---|---|
| | | DSR- | DSR- | DSR- | DSR- |
| animal No. | PBS | 0103432 | 0103299 | 0103300 | 0103301 |
| 1 | 91.20 | 109.70 | 34.00 | 76.90 | 26.70 |
| 2 | 105.90 | 91.40 | 46.50 | 32.30 | 25.20 |
| 3 | 107.10 | 110.40 | 26.10 | 33.10 | 48.60 |
| 4 | 98.30 | 97.30 | 30.00 | 30.10 | 14.60 |
| 5 | N.D | 92.60 | 38.20 | 28.00 | 17.80 |
| 6 | 97.60 | 91.70 | 30.60 | 24.20 | 22.50 |
| Mean | 100.02 | 98.85 | 34.23 | 37.43 | 25.90 |

Table 126C. Shows 00 mouse APP mRNA remaining relative to PBS control in the hippocampus 16 weeks post dosing. N=6. N.D.: Not determined.

| | % remaining of mAPP mRNA | | | | |
|---|---|---|---|---|---|
| | | DSR- | DSR- | DSR- | DSR- |
| animal No. | PBS | 0103432 | 0103299 | 0103300 | 0103301 |
| 1 | 98.00 | 101.60 | 23.70 | 16.10 | 10.50 |
| 2 | 113.50 | 91.70 | 33.80 | 13.00 | 11.50 |
| 3 | 85.60 | 75.60 | 41.10 | 11.70 | 32.80 |
| 4 | 92.40 | 101.20 | 40.90 | 26.20 | 6.40 |
| 5 | N.D | 89.30 | 28.80 | 22.40 | 7.80 |
| 6 | 110.60 | 82.40 | 33.70 | 9.20 | 7.40 |
| Mean | 100.02 | 90.30 | 33.67 | 16.43 | 12.73 |

Table 126D. Shows 0% mouse APP mRNA remaining relative to PBS control in the cortex 16 weeks post dosing. N=6. N.D.: Not determined.

| | % remaining of mAPP mRNA | | | | |
|---|---|---|---|---|---|
| | | DSR- | DSR- | DSR- | DSR- |
| animal No. | PBS | 0103432 | 0103299 | 0103300 | 0103301 |
| 1 | 113.70 | 108.00 | 34.90 | 40.00 | 21.00 |
| 2 | 101.30 | 117.60 | 50.50 | 31.70 | 40.10 |
| 3 | 106.20 | 103.60 | 61.30 | 29.60 | 32.80 |
| 4 | 99.10 | 90.00 | 49.60 | 43.20 | 11.60 |
| 5 | N.D | 98.00 | 50.00 | 33.80 | 18.10 |
| 6 | 79.70 | 88.80 | 47.90 | 23.20 | 20.20 |
| Mean | 100.00 | 101.00 | 49.03 | 33.58 | 23.97 |

Table 126E. Shows 00 mouse APP mRNA remaining relative to PBS control in the spinal cord 16 weeks post dosing. N=6. N.D.: Not determined.

| | % remaining of mAPP mRNA | | | | |
|---|---|---|---|---|---|
| | | DSR- | DSR- | DSR- | DSR- |
| animal No. | PBS | 0103432 | 0103299 | 0103300 | 0103301 |
| 1 | 104.20 | 88.00 | 39.50 | 22.40 | 26.00 |
| 2 | 97.00 | 84.30 | 38.30 | 17.00 | 51.50 |
| 3 | 90.00 | 118.30 | 48.70 | 15.00 | 14.10 |
| 4 | 98.30 | 113.40 | 61.90 | 19.00 | 15.90 |
| 5 | N.D | 99.70 | 49.50 | 24.50 | 20.20 |
| 6 | 110.40 | 112.40 | 59.20 | 17.50 | 20.40 |
| Mean | 99.98 | 102.68 | 49.52 | 19.23 | 24.68 |

Table 126F. Shows 00 mouse APP mRNA remaining relative to PBS control in the cerebellum 16 weeks post dosing. N=6. N.D.: Not determined.

| | % remaining of mAPP mRNA | | | | |
|---|---|---|---|---|---|
| | | DSR- | DSR- | DSR- | DSR- |
| animal No. | PBS | 0103432 | 0103299 | 0103300 | 0103301 |
| 1 | 92.50 | 102.00 | 36.50 | 26.30 | 17.30 |
| 2 | 89.40 | 86.60 | 29.50 | 29.60 | 21.10 |
| 3 | 91.00 | 89.60 | 38.10 | 24.50 | 51.30 |
| 4 | 109.50 | 114.10 | 32.60 | 26.30 | 24.70 |
| 5 | N.D | 108.70 | 41.70 | 25.90 | 29.00 |
| 6 | 117.70 | 105.00 | 49.00 | 33.40 | 24.10 |
| Mean | 100.02 | 101.00 | 37.90 | 27.67 | 27.92 |

Table 126G. Shows the accumulation of antisense strand in the cortex at 8 weeks, 12 weeks and 16 weeks post dosing. N=5. N.D.: Not determined.

| | animal | Cortex Guide Strand PK (ug oligo/g tissue) | | | | |
|---|---|---|---|---|---|---|
| | No. | PBS | DSR-0103432 | DSR-0103299 | DSR-0103300 | DSR-0103301 |
| 8 weeks | 1 | 0.00 | 0.63 | 1.66 | 1.23 | 2.47 |
| post | 2 | 0.00 | 0.71 | 2.37 | 2.24 | 2.08 |
| dosing | 3 | 0.00 | 0.04 | 2.09 | 0.82 | 2.94 |
| | 4 | 0.00 | 1.29 | 2.57 | 2.93 | 8.67 |
| | 5 | 0.00 | 0.28 | 7.81 | 2.52 | 9.00 |
| | Mean | 0.00 | 0.59 | 3.30 | 1.95 | 5.03 |
| 12 weeks | 1 | 0.01 | 3.77 | 3.54 | 4.99 | 3.20 |
| post | 2 | 0.00 | 1.25 | 1.76 | 1.59 | 4.92 |
| dosing | 3 | 0.00 | 3.84 | 1.90 | 1.45 | N.D. |
| | 4 | 0.00 | 4.20 | 0.56 | 0.78 | 4.01 |
| | 5 | 0.07 | 0.48 | 1.86 | 12.68 | 1.32 |
| | Mean | 0.02 | 2.71 | 1.92 | 4.30 | 3.36 |
| 16 weeks | 1 | 0.00 | 0.66 | 1.75 | 0.73 | 1.73 |
| post | 2 | 0.00 | 0.94 | 0.97 | 2.73 | 7.52 |
| dosing | 3 | 0.00 | 0.95 | 0.77 | 0.73 | 1.57 |
| | 4 | N.D. | 0.67 | 1.58 | 2.13 | 2.56 |
| | 5 | 0.00 | 0.98 | 2.14 | 3.03 | 1.93 |
| | Mean | 0.00 | 0.84 | 1.44 | 1.87 | 3.06 |

Table 126H. Shows the accumulation of antisense strand in the spinal cord at 8 weeks, 12 weeks and 16 weeks post dosing. N=6. N.D.: Not determined.

| | | Spinal cord Guide Strand PK (ug oligo/g tissue) | | | | |
|---|---|---|---|---|---|---|
| | Animal | PBS | DSR-0103432 | DSR-0103299 | DSR-0103300 | DSR-0103301 |
| 8 weeks | 1 | 0.00 | 0.25 | 0.76 | 1.26 | 1.94 |
| post | 2 | 0.00 | 0.20 | N.D. | 0.07 | 2.01 |
| dosing | 3 | 0.00 | 0.21 | 0.94 | 1.65 | 2.53 |
| | 4 | 0.00 | 0.63 | 0.85 | 0.41 | 1.41 |
| | 5 | 0.00 | 0.53 | 1.13 | 2.81 | 2.04 |
| | 6 | 0.00 | 0.07 | 0.69 | 2.28 | 3.13 |
| | Mean | 0.00 | 0.31 | 0.87 | 1.41 | 2.18 |
| 12 weeks | 1 | 0.00 | 0.24 | 0.61 | 1.01 | 2.15 |
| post | 2 | 0.00 | 0.32 | 0.57 | 0.53 | 3.01 |
| dosing | 3 | 0.00 | 0.28 | 0.97 | 1.63 | 3.13 |
| | 4 | 0.00 | 0.32 | 0.88 | 1.55 | N.D. |
| | 5 | 0.00 | 0.28 | 0.31 | 0.74 | 2.69 |
| | 6 | 0.00 | 0.05 | 0.26 | 2.38 | 2.56 |
| | Mean | 0.00 | 0.25 | 0.60 | 1.31 | 2.71 |
| 16 weeks | 1 | 0.00 | 0.14 | 0.31 | 0.87 | 0.84 |
| post | 2 | 0.00 | 0.10 | 0.49 | 0.44 | 0.32 |
| dosing | 3 | 0.00 | 0.09 | 0.45 | 1.34 | 1.66 |
| | 4 | 0.00 | 0.20 | 0.24 | 0.64 | 1.43 |
| | 5 | N.D. | 0.12 | 0.24 | 0.95 | 1.41 |
| | 6 | 0.00 | 0.20 | 0.32 | 1.25 | 0.99 |
| | Mean | 0.00 | 0.14 | 0.34 | 0.92 | 1.11 |

Table 126I. Shows the accumulation of antisense strand in the hippocampus at 8 weeks, 12 weeks and 16 weeks post dosing. N=5. N.D.: Not determined.

| | animal | Hippocampus Guide Strand PK (ug oligo/g tissue) | | | | |
|---|---|---|---|---|---|---|
| | No. | PBS | DSR-0103432 | DSR-0103299 | DSR-0103300 | DSR-0103301 |
| 8 weeks | 1 | 0.00 | 0.67 | 1.45 | 2.30 | 5.95 |
| post | 2 | 0.00 | 0.88 | 2.15 | 3.09 | 5.67 |
| dosing | 3 | 0.00 | 2.37 | 1.15 | 5.31 | 8.51 |
| | 4 | 0.00 | 1.07 | 7.34 | 5.48 | 14.08 |
| | 5 | 0.00 | 0.18 | 10.66 | 5.44 | 18.04 |
| | Mean | 0.00 | 1.03 | 4.55 | 4.32 | 10.45 |
| 12 weeks | 1 | 0.00 | 2.57 | 1.87 | 5.37 | 3.93 |
| post | 2 | 0.00 | 1.26 | 2.94 | 2.27 | 6.44 |
| dosing | 3 | 0.00 | 2.68 | 2.41 | 1.50 | N.D. |
| | 4 | 0.00 | 6.98 | 0.65 | 0.97 | 7.95 |
| | 5 | 0.00 | 0.39 | 2.08 | 23.47 | 2.51 |
| | Mean | 0.00 | 2.78 | 1.99 | 6.72 | 5.21 |
| 16 weeks | 1 | 0.00 | 0.47 | 1.52 | 2.18 | 2.45 |
| post | 2 | 0.00 | 1.06 | 1.36 | 5.13 | 4.29 |
| dosing | 3 | 0.00 | 1.00 | 0.88 | 1.26 | 1.34 |
| | 4 | 0.00 | 0.57 | 1.39 | 2.38 | 6.67 |
| | 5 | 0.00 | 0.91 | 2.22 | 3.45 | 1.72 |
| | Mean | 0.00 | 0.80 | 1.47 | 2.88 | 3.29 |

Table 126J. Shows the accumulation of antisense strand in the striatum at 8 weeks, 12 weeks and 16 weeks post dosing. N=5. N.D.: Not determined.

| | animal | Striatum Guide Strand PK (ug oligo/g tissue) | | | | |
|---|---|---|---|---|---|---|
| | No. | PBS | DSR-0103432 | DSR-0103299 | DSR-0103300 | DSR-0103301 |
| 8 weeks | 1 | 0.00 | 0.52 | 0.68 | 1.39 | 2.23 |
| post | 2 | 0.00 | 0.96 | 1.59 | 2.02 | 2.68 |
| dosing | 3 | 0.00 | 1.53 | 1.51 | 1.28 | 2.47 |
| | 4 | 0.00 | 0.96 | 1.46 | 2.21 | 3.74 |
| | 5 | 0.00 | 0.22 | 2.59 | 2.76 | 4.02 |
| | Mean | 0.00 | 0.84 | 1.57 | 1.93 | 3.03 |
| 12 weeks | 1 | 0.01 | 1.89 | 1.64 | 3.65 | 7.41 |
| post | 2 | 0.00 | 0.91 | 1.13 | 1.83 | 3.74 |
| dosing | 3 | 0.00 | 2.66 | 1.50 | 1.79 | N.D |
| | 4 | 0.00 | 3.15 | 0.59 | 0.44 | 4.27 |
| | 5 | 0.01 | 0.42 | 1.58 | 6.46 | 1.22 |
| | Mean | 0.00 | 1.80 | 1.29 | 2.83 | 4.16 |
| 16 weeks | 1 | 0.01 | 0.85 | 0.76 | 0.33 | 2.00 |
| post | 2 | 0.01 | 0.56 | 0.58 | 1.36 | 3.90 |
| dosing | 3 | 0.01 | 1.22 | 0.60 | 0.52 | 0.61 |
| | 4 | N.D | 0.57 | 0.67 | 1.87 | 2.15 |
| | 5 | 0.01 | 0.38 | 2.34 | 1.48 | 2.45 |
| | Mean | 0.01 | 0.72 | 0.99 | 1.11 | 2.22 |

Table 126K. Shows changes in mouse AIF1 mRNA relative to PBS control in the spinal cord 8 weeks, 12 weeks and 16 weeks post dosing. N=6. N.D.: Not determined.

| | Animal | PBS | DSR-0103432 | DSR-0103299 | DSR-0103300 | DSR-0103301 |
|---|---|---|---|---|---|---|
| 8 weeks | 1 | 1.04 | 1.21 | 1.15 | 1.05 | 1.54 |
| post | 2 | 0.99 | 0.59 | 0.67 | 1.25 | 0.92 |
| dosing | 3 | 1.05 | 1.15 | 1.41 | 1.49 | 1.07 |
| | 4 | 0.84 | 0.95 | 1.06 | 1.33 | 0.91 |
| | 5 | 1.20 | 1.34 | 1.22 | 1.34 | 1.41 |
| | 6 | 0.89 | 1.09 | 0.89 | 1.24 | 1.50 |
| | Mean | 1.00 | 1.06 | 1.07 | 1.28 | 1.23 |
| 12 weeks | 1 | 1.39 | 0.82 | 1.12 | 1.78 | 1.06 |
| post | 2 | 0.95 | 0.75 | 0.61 | 0.71 | 0.65 |
| dosing | 3 | 0.77 | 0.59 | 0.78 | 0.58 | 0.67 |
| | 4 | 0.97 | 0.85 | 0.92 | 0.77 | N.D |
| | 5 | 1.08 | 0.85 | 0.93 | 0.66 | 0.63 |
| | 6 | 0.83 | 1.52 | 1.01 | 0.80 | 0.92 |
| | Mean | 1.00 | 0.90 | 0.89 | 0.88 | 0.79 |
| 16 weeks | 1 | 1.32 | 1.10 | 1.09 | 0.78 | 1.01 |
| post | 2 | 0.97 | 0.61 | 1.03 | 0.96 | 1.04 |
| dosing | 3 | 0.79 | 1.13 | 0.92 | 0.84 | 0.87 |
| | 4 | 0.91 | 0.78 | 1.05 | 0.80 | 0.68 |
| | 5 | N.D | 0.75 | 1.08 | 1.02 | 1.01 |
| | 6 | 1.01 | 1.00 | 0.94 | 0.66 | 0.86 |
| | Mean | 1.00 | 0.90 | 1.02 | 0.84 | 0.91 |

Table 126L. Shows changes in mouse GFAP mRNA relative to PBS control in the spinal cord 8 weeks, 12 weeks and 16 weeks post dosing. N=6. N.D.: Not determined.

| | | | DSR- | DSR- | DSR- | DSR- |
|---|---|---|---|---|---|---|
| | Animal | PBS | 0103432 | 0103299 | 0103300 | 0103301 |
| 8 | 1 | 0.95 | 1.09 | 1.23 | 1.09 | 1.26 |
| weeks | 2 | 0.83 | 0.71 | 0.73 | 1.19 | 0.91 |
| post | 3 | 1.06 | 0.92 | 1.50 | 1.40 | 1.15 |
| dosing | 4 | 0.83 | 1.06 | 0.99 | 1.18 | 1.06 |
| | 5 | 1.29 | 1.55 | 1.35 | 1.31 | 1.36 |
| | 6 | 1.04 | 1.33 | 0.82 | 1.52 | 1.26 |
| | Mean | 1.00 | 1.11 | 1.10 | 1.28 | 1.17 |
| 12 | 1 | 1.36 | 1.00 | 1.09 | 1.48 | 1.00 |
| weeks | 2 | 0.91 | 0.80 | 0.75 | 0.95 | 0.74 |
| post | 3 | 0.86 | 0.71 | 1.01 | 0.84 | 0.87 |
| dosing | 4 | 0.95 | 1.00 | 1.28 | 0.97 | N.D |
| | 5 | 0.98 | 0.96 | 1.37 | 0.68 | 0.79 |
| | 6 | 0.94 | 1.42 | 1.31 | 0.97 | 0.81 |
| | Mean | 1.00 | 0.98 | 1.14 | 0.98 | 0.85 |
| 16 | 1 | 1.04 | 1.27 | 1.23 | 0.87 | 1.17 |
| weeks | 2 | 1.02 | 0.55 | 1.04 | 0.97 | 1.08 |
| post | 3 | 0.76 | 1.25 | 1.15 | 1.10 | 0.95 |
| dosing | 4 | 0.94 | 0.91 | 1.24 | 1.01 | 0.96 |
| | 5 | N.D | 0.88 | 1.49 | 1.33 | 1.44 |
| | 6 | 1.23 | 1.21 | 1.47 | 0.94 | 1.04 |
| | Mean | 1.00 | 1.01 | 1.27 | 1.04 | 1.11 |

Table 126M. Shows changes in mouse AIF1 mRNA relative to PBS control in the cerebellum 8 weeks, 12 weeks and 16 weeks post dosing. N=6. N.D.: Not determined.

| | | | DSR- | DSR- | DSR- | DSR- |
|---|---|---|---|---|---|---|
| | Animal | PBS | 0103432 | 0103299 | 0103300 | 0103301 |
| 8 | 1 | 1.01 | 0.71 | 0.72 | 0.75 | 0.91 |
| weeks | 2 | 0.99 | 0.63 | 1.01 | 0.63 | 0.91 |
| post | 3 | 0.92 | 0.95 | 1.11 | 0.68 | 1.07 |
| dosing | 4 | 1.10 | 0.72 | 1.06 | 0.67 | 0.99 |
| | 5 | 0.91 | 0.46 | 1.26 | 0.76 | 1.42 |
| | 6 | 1.07 | 0.96 | 1.29 | 0.65 | 0.97 |
| | Mean | 1.00 | 0.74 | 1.07 | 0.69 | 1.05 |
| 12 | 1 | 0.98 | 0.84 | 1.46 | 0.90 | 1.06 |
| weeks | 2 | 1.03 | 0.75 | 1.42 | 1.01 | 1.20 |
| post | 3 | 0.94 | 0.75 | 1.25 | 1.50 | 1.42 |
| dosing | 4 | 1.08 | 0.92 | 1.46 | 1.24 | |
| | 5 | 1.02 | 0.94 | 1.37 | 1.16 | 1.05 |
| | 6 | 0.94 | 1.63 | 0.79 | 1.22 | 0.78 |
| | Mean | 1.00 | 0.97 | 1.29 | 1.17 | 1.10 |
| 16 | 1 | 1.55 | 0.87 | 0.99 | 0.62 | 0.88 |
| weeks | 2 | 0.89 | 0.79 | 1.17 | 0.99 | 0.82 |
| post | 3 | 0.92 | 0.90 | 0.91 | 0.71 | 0.81 |
| dosing | 4 | 0.84 | 0.95 | 0.72 | 0.64 | 0.90 |
| | 5 | N.D | 0.84 | 1.07 | 0.64 | 0.81 |
| | 6 | 0.79 | 0.98 | 0.91 | 0.72 | 0.69 |
| | Mean | 1.00 | 0.89 | 0.96 | 0.72 | 0.82 |

Table 126N. Shows changes in mouse GFAP mRNA relative to PBS control in the cerebellum 8 weeks, 12 weeks and 16 weeks post dosing. N=6. N.D. Not determined.

| | | | DSR- | DSR- | DSR- | DSR- |
|---|---|---|---|---|---|---|
| | Animal | PBS | 0103432 | 0103299 | 0103300 | 0103301 |
| 8 | 1 | 1.17 | 1.37 | 0.79 | 1.33 | 1.63 |
| weeks | 2 | 0.63 | 1.36 | 0.96 | 1.30 | 0.97 |
| post | 3 | 0.92 | 1.33 | 1.05 | 1.68 | 1.27 |
| dosing | 4 | 0.96 | 1.16 | 0.85 | 1.24 | 0.80 |
| | 5 | 0.95 | 0.76 | 1.03 | 1.55 | 1.41 |
| | 6 | 1.36 | 1.13 | 0.92 | 1.16 | 0.96 |
| | Mean | 1.00 | 1.18 | 0.93 | 1.38 | 1.17 |
| 12 | 1 | 0.70 | 1.17 | 0.35 | 1.27 | 0.98 |
| weeks | 2 | 1.02 | 0.97 | 0.91 | 1.42 | 1.10 |
| post | 3 | 0.92 | 1.10 | 0.85 | 0.93 | 1.42 |
| dosing | 4 | 1.10 | 1.35 | 0.75 | 1.49 | N.D |
| | 5 | 1.01 | 0.99 | 1.14 | 1.29 | 1.08 |
| | 6 | 1.24 | 1.27 | 1.14 | 1.02 | 0.97 |
| | Mean | 1.00 | 1.14 | 0.86 | 1.23 | 1.11 |
| 16 | 1 | 0.98 | 1.06 | 1.10 | 0.86 | 1.15 |
| weeks | 2 | 0.96 | 0.80 | 1.08 | 1.26 | 1.09 |
| post | 3 | 1.01 | 0.86 | 1.21 | 1.15 | 0.95 |
| dosing | 4 | 0.99 | 1.18 | 0.69 | 0.93 | 1.21 |
| | 5 | N.D | 1.04 | 1.40 | 0.90 | 1.11 |
| | 6 | 1.06 | 1.18 | 1.37 | 1.20 | 1.00 |
| | Mean | 1.00 | 1.02 | 1.14 | 1.05 | 1.08 |

Table 126O. Shows changes in mouse PCP2 mRNA relative to PBS control in the cerebellum 8 weeks, 12 weeks and 16 weeks post dosing. N=6. N.D.: Not determined.

| | | | DSR- | DSR- | DSR- | DSR- |
|---|---|---|---|---|---|---|
| | Animal | PBS | 0103432 | 0103299 | 0103300 | 0103301 |
| 8 weeks | 1 | 1.10 | 1.06 | 1.04 | 1.46 | 1.87 |
| post | 2 | 0.99 | 1.33 | 1.30 | 1.27 | 1.37 |
| dosing | 3 | 1.05 | 1.10 | 1.31 | 1.09 | 1.27 |
| | 4 | 1.09 | 1.32 | 1.08 | 1.30 | 1.17 |
| | 5 | 0.95 | 1.19 | 1.26 | 1.26 | 1.14 |
| | 6 | 0.82 | 1.07 | 1.12 | 1.20 | 0.93 |
| | Mean | 1.00 | 1.18 | 1.19 | 1.26 | 1.29 |
| 12 | 1 | 0.85 | 1.20 | 0.04 | 0.68 | 0.85 |
| weeks | 2 | 0.88 | 0.92 | 0.84 | 1.23 | 1.14 |
| post | 3 | 0.98 | 1.07 | 0.82 | 0.00 | 1.09 |
| dosing | 4 | 1.18 | 1.33 | 0.84 | 1.29 | N.D |
| | 5 | 1.04 | 1.25 | 1.01 | 1.27 | 1.29 |
| | 6 | 1.08 | 0.94 | 1.24 | 1.16 | 1.06 |
| | Mean | 1.00 | 1.12 | 0.80 | 0.94 | 1.09 |
| 16 | 1 | 0.78 | 0.92 | 1.03 | 0.98 | 0.72 |
| weeks | 2 | 0.90 | 0.84 | 1.09 | 0.96 | 1.14 |
| post | 3 | 0.98 | 0.45 | 0.68 | 0.80 | 1.13 |
| dosing | 4 | 1.14 | 0.96 | 0.97 | 0.91 | 0.90 |
| | 5 | N.D | 0.57 | 1.06 | 1.02 | 1.26 |
| | 6 | 1.20 | 1.01 | 1.18 | 0.83 | 1.64 |
| | Mean | 1.00 | 0.79 | 1.00 | 0.92 | 1.13 |

Table 126P. Shows changes in mouse GRID2 mRNA relative to PBS control in the cerebellum 8 weeks, 12 weeks and 16 weeks post dosing. N=6. N.D.: Not determined.

| | | | DSR- | DSR- | DSR- | DSR- |
|---|---|---|---|---|---|---|
| | Animal | PBS | 0103432 | 0103299 | 0103300 | 0103301 |
| 8 | 1 | 1.15 | 0.97 | 0.89 | 1.09 | 1.32 |
| weeks | 2 | 1.07 | 1.03 | 1.12 | 0.93 | 0.96 |
| post | 3 | 0.92 | 1.05 | 1.14 | 0.95 | 1.18 |
| dosing | 4 | 1.05 | 0.86 | 0.91 | 0.99 | 0.98 |
| | 5 | 0.94 | 0.70 | 1.14 | 0.86 | 1.14 |
| | 6 | 0.87 | 0.78 | 1.02 | 0.78 | 0.83 |
| | Mean | 1.00 | 0.90 | 1.03 | 0.93 | 1.07 |
| 12 | 1 | 0.93 | 1.00 | 0.14 | 0.63 | 0.92 |
| weeks | 2 | 1.05 | 0.88 | 0.88 | 1.08 | 1.26 |
| post | 3 | 0.87 | 0.93 | 0.82 | 0.08 | 1.03 |
| dosing | 4 | 1.12 | 0.99 | 0.82 | 1.05 | N.D |
| | 5 | 0.96 | 1.02 | 0.94 | 1.12 | 1.06 |
| | 6 | 1.07 | 0.94 | 1.11 | 1.23 | 0.82 |
| | Mean | 1.00 | 0.96 | 0.78 | 0.87 | 1.02 |
| 16 | 1 | 0.92 | 0.90 | 1.19 | 0.90 | 0.82 |
| weeks | 2 | 0.94 | 0.83 | 1.10 | 0.98 | 1.30 |
| post | 3 | 0.95 | 0.58 | 0.77 | 0.82 | 1.15 |
| dosing | 4 | 0.98 | 0.94 | 0.87 | 0.96 | 0.81 |
| | 5 | N.D | 0.76 | 0.96 | 1.15 | 0.97 |
| | 6 | 1.20 | 1.16 | 1.01 | 1.09 | 1.02 |
| | Mean | 1.00 | 0.86 | 0.98 | 0.98 | 1.01 |

Table 126Q. Shows changes in mouse CALB1 mRNA relative to PBS control in the cerebellum 8 weeks, 12 weeks and 16 weeks post dosing. N=6. N.D.: Not determined.

| | | | DSR- | DSR- | DSR- | DSR- |
|---|---|---|---|---|---|---|
| | Animal | PBS | 0103432 | 0103299 | 0103300 | 0103301 |
| 8 | 1 | 1.29 | 1.01 | 0.96 | 1.10 | 1.75 |
| weeks | 2 | 0.98 | 0.98 | 1.30 | 1.00 | 0.84 |
| post | 3 | 0.92 | 1.30 | 1.06 | 1.34 | 1.29 |
| dosing | 4 | 0.75 | 1.47 | 0.98 | 1.50 | 0.66 |
| | 5 | 1.20 | 0.75 | 1.40 | 1.03 | 1.13 |
| | 6 | 0.87 | 1.32 | 1.07 | 0.96 | 0.56 |
| | Mean | 1.00 | 1.14 | 1.13 | 1.16 | 1.04 |
| 12 | 1 | 0.91 | 1.17 | 0.13 | 0.82 | 0.82 |
| weeks | 2 | 0.96 | 1.11 | 0.89 | 1.25 | 1.02 |
| post | 3 | 0.90 | 1.19 | 0.85 | 0.08 | 0.88 |
| dosing | 4 | 1.04 | 1.25 | 0.79 | 1.27 | N.D |
| | 5 | 1.03 | 1.10 | 1.08 | 1.27 | 0.88 |
| | 6 | 1.17 | 1.05 | 1.23 | 1.19 | 0.90 |
| | Mean | 1.00 | 1.14 | 0.83 | 0.98 | 0.90 |
| 16 | 1 | 0.94 | 1.71 | 1.33 | 1.32 | 0.82 |
| weeks | 2 | 0.94 | 1.49 | 1.21 | 1.56 | 1.27 |
| post | 3 | 0.99 | 0.85 | 0.76 | 1.06 | 1.11 |
| dosing | 4 | 1.11 | 1.27 | 1.06 | 1.31 | 0.77 |
| | 5 | N.D | 0.99 | 1.03 | 1.29 | 0.82 |
| | 6 | 1.01 | 1.37 | 1.00 | 1.12 | 1.10 |
| | Mean | 1.00 | 1.28 | 1.06 | 1.28 | 0.98 |

Table 126R. Shows changes in mouse serum NfL relative to PBS control 16 weeks post dosing. N=6. N.D.: Not determined.

| | | DSR- | DSR- | DSR- | DSR- |
|---|---|---|---|---|---|
| Animal | PBS | 0103432 | 0103299 | 0103300 | 0103301 |
| 1 | 930.4 | 532.9 | 1185.1 | 556.4 | 627.0 |
| 2 | 767.5 | 367.3 | 1346.2 | 673.8 | 199.5 |
| 3 | 579.9 | 1092.7 | 1277.4 | 461.4 | 1000.1 |
| 4 | 414.8 | 953.6 | 438.4 | 907.2 | 484.8 |
| 5 | N.D. | 697.4 | 1023.1 | 1069.5 | 603.6 |
| 6 | 720.7 | 391.1 | 462.2 | 790.1 | 391.1 |
| Mean | 682.7 | 672.5 | 955.4 | 743.1 | 551.0 |

### Provided Oligonucleotides and Compositions are Active In Situ

Various siRNAs for APP protein knockdown in brain tissues were designed and constructed. A number of siRNAs were tested in situ using an Immunohistochemistry (IHC) Assay. Example protocol for Immunohistochemistry Assay: Mouse brain hemisphere biopsies were fixed and embedded in paraffin as described above. 10 um sections were cut and followed by standard IHC staining methods using anti-APP Rabbit mAb #19389 (E3F3P) and isotype control Rabbit mAb #3900 (DA1E) (Cell Signaling Technology), followed by Polymer-HRP 2nd Detection antibody, and developed with DAB (brown) for 10 min and counterstained with hematoxylin (blue). The representative digital images were generated using a Zeiss Axio Observer microscope (Zeiss, Thornwood, NY, USA) under brightfield.

IHC results show potent and sustainable APP KD across all the different brain regions of interest, including Frontal Cx, Striatum, Hippocampus, Brain stem (MidBrain and Medulla) and Cerebellum, for up to 16 weeks after single ICV administration of siRNA. APP protein prominently expressed in Neuron cells in all key brain regions, and potent APP protein KD were observed in these Neuron cells. Although only detectable level of APP protein was seen in Oligodendrocytes, and very low or no detectable APP protein expression in Astrocytes and Microglia. Furthermore, no differences of APP knockdown were observed between two time-points of 12 weeks and 16 weeks for each individual siRNA compounds.

### Example 14. Provided Oligonucleotides and Compositions are Active In Vivo

All animal procedures were performed under IACUC guidelines. To evaluate the potency and liver exposure of provided oligonucleotides and compositions, male 8-10 weeks of age C57BL/6 mice were dose at 5 mg/kg on Day 0 by subcutaneous administration. Animals were euthanized on Day 14 or 28 by CO2 asphyxiation followed by thoracotomy and terminal blood collection. After cardiac perfusion with PBS, various tissue samples were harvested and flash-frozen in dry ice. Total RNA was extracted using RNeasy 96 kit (Qiagen), after tissue lysis with TRIzol and chloroform. cDNA production from RNA samples were performed using High-Capacity cDNA Reverse Transcription kit (Thermo Fisher) following manufacturer's instructions and qPCR analysis performed in CFX System using iQ Multiplex Powermix (Bio-Rad for mouse SOD1 mRNA, the following qPCR assay were utilized: mouse Sod1 from IDT (Integrated DNA Technologies) qPCR assay ID Mm.PT.58.12368303. Mouse HPRT was used as normalizer (Forward 5′CAAACTTTGCTTTCCCTGGTT3′, Reverse 5′TGGCCTGTATCCAACACTTC3′, Probe 5′/5HEX/ACCAGCAAG/Zen/CTTGCAACCTTAACC/3IABkFQ/3′. Oligonucleotide accumulation was determined by sandwich ELISA.

Table 127. shows % mouse SOD1 mRNA remaining relative to PBS control in the quad 14 days post dosing. N=5. N.D.: Not determined.

| | animal No. | | | | | |
|---|---|---|---|---|---|---|
| siRNA | 1 | 2 | 3 | 4 | 5 | Mean |
| PBS | 113.30 | 101.49 | 99.58 | 95.38 | 90.26 | 100.00 |
| DSR- | 81.53 | 85.53 | 92.84 | 76.08 | 78.33 | 82.86 |
| 0103301 | | | | | | |
| DSR- | 71.27 | 66.66 | 75.66 | 70.10 | 75.14 | 71.76 |
| 0103966 | | | | | | |
| DSR- | 63.81 | 65.26 | 58.77 | 59.10 | 61.60 | 61.71 |
| 0103967 | | | | | | |
| DSR- | 59.72 | 62.65 | 65.81 | 61.38 | 57.37 | 61.38 |
| 0103968 | | | | | | |
| DSR- | 42.78 | 53.38 | 46.17 | 61.90 | 56.02 | 52.05 |
| 0103969 | | | | | | |
| DSR- | 71.38 | 72.15 | 67.96 | 68.31 | 56.63 | 67.28 |
| 0103970 | | | | | | |
| DSR- | 60.24 | 56.92 | 61.07 | 65.48 | 56.70 | 60.08 |
| 0103971 | | | | | | |
| DSR- | 61.59 | 61.97 | 65.67 | 62.31 | 67.48 | 63.80 |
| 0103972 | | | | | | |
| DSR- | 73.24 | 77.14 | 82.19 | 79.50 | 76.85 | 77.78 |
| 0103973 | | | | | | |

Table 128. shows % mouse SOD1 mRNA remaining relative to PBS control in the liver 14 days post dosing. N=5. N.D.: Not determined.

| | animal No. | | | | | |
|---|---|---|---|---|---|---|
| siRNA | 1 | 2 | 3 | 4 | 5 | Mean |
| PBS | 99.87 | 105.72 | 94.66 | 107.34 | 92.41 | 100.00 |
| DSR- | 108.78 | 100.15 | 112.64 | 92.71 | 89.65 | 100.79 |
| 0103301 | | | | | | |
| DSR- | 92.95 | 82.34 | 75.88 | 63.38 | 50.96 | 73.10 |
| 0103966 | | | | | | |
| DSR- | 66.05 | 47.41 | 49.65 | 41.66 | 29.74 | 46.90 |
| 0103967 | | | | | | |
| DSR- | 80.98 | 57.11 | 60.94 | 50.65 | 33.93 | 56.72 |
| 0103968 | | | | | | |
| DSR- | 57.31 | 57.10 | 36.40 | 38.92 | 40.18 | 45.98 |
| 0103969 | | | | | | |
| DSR- | 89.18 | 70.46 | 85.49 | 44.57 | 59.49 | 69.84 |
| 0103970 | | | | | | |
| DSR- | 63.71 | 59.85 | 44.64 | 52.05 | 44.59 | 52.97 |
| 0103971 | | | | | | |
| DSR- | 41.51 | 49.36 | 49.34 | 46.58 | 41.21 | 45.60 |
| 0103972 | | | | | | |
| DSR- | 77.74 | 75.75 | 78.11 | 72.26 | 76.41 | 76.05 |
| 0103973 | | | | | | |

Table 129. shows % mouse SOD1 mRNA remaining relative to PBS control in the lung 14 days post dosing. N=5. N.D.: Not determined.

| | animal No. | | | | | |
|---|---|---|---|---|---|---|
| siRNA | 1 | 2 | 3 | 4 | 5 | Mean |
| PBS | 144.11 | 100.58 | 54.30 | 117.86 | 83.14 | 100.00 |
| DSR- | 111.07 | 65.80 | 93.41 | 103.28 | 99.48 | 94.61 |
| 0103301 | | | | | | |
| DSR- | 89.60 | 103.54 | 102.34 | 97.63 | 97.62 | 98.15 |
| 0103966 | | | | | | |
| DSR- | 127.70 | 82.95 | 74.17 | 100.48 | 68.01 | 90.66 |
| 0103967 | | | | | | |
| DSR- | 95.88 | 104.18 | 96.00 | 106.81 | 78.98 | 96.37 |
| 0103968 | | | | | | |
| DSR- | 71.26 | 86.56 | 75.25 | 86.95 | 90.04 | 82.01 |
| 0103969 | | | | | | |
| DSR- | 120.52 | 80.04 | 100.37 | 70.25 | 107.07 | 95.65 |
| 0103970 | | | | | | |
| DSR- | 127.88 | 106.71 | 85.71 | 125.39 | 87.48 | 106.64 |
| 0103971 | | | | | | |
| DSR- | 44.81 | 78.44 | 85.17 | 29.87 | 71.07 | 61.87 |
| 0103972 | | | | | | |
| DSR- | 80.77 | 90.34 | 92.41 | 71.71 | 75.12 | 82.07 |
| 0103973 | | | | | | |

Table 130. shows % mouse SOD1 mRNA remaining relative to PBS control in the white adipose tissue 14 days post dosing. N=5. N.D.: Not determined.

| | animal No. | | | | | |
|---|---|---|---|---|---|---|
| siRNA | 1 | 2 | 3 | 4 | 5 | Mean |
| PBS | 62.01 | 149.52 | 92.68 | 79.84 | 115.95 | 100.00 |
| DSR- | 119.78 | 137.31 | 105.19 | 74.89 | 118.17 | 111.07 |
| 0103301 | | | | | | |
| DSR- | 106.64 | 137.95 | 110.05 | 117.07 | 107.04 | 115.75 |
| 0103966 | | | | | | |
| DSR- | 69.85 | 99.61 | 63.45 | 81.87 | 88.47 | 80.65 |
| 0103967 | | | | | | |
| DSR- | 65.54 | 84.67 | 51.20 | 48.11 | 28.31 | 55.57 |
| 0103968 | | | | | | |
| DSR- | 8.87 | 22.79 | 18.96 | 25.58 | 30.18 | 21.27 |
| 0103969 | | | | | | |
| DSR- | 76.04 | 75.88 | 67.01 | 53.61 | 57.89 | 66.08 |
| 0103970 | | | | | | |
| DSR- | 32.94 | 26.87 | 51.32 | 36.73 | 45.84 | 38.74 |
| 0103971 | | | | | | |
| DSR- | 44.25 | 52.44 | 58.82 | 39.40 | 33.26 | 45.63 |
| 0103972 | | | | | | |
| DSR- | 70.48 | 93.79 | 45.42 | 57.55 | 53.03 | 64.05 |
| 0103973 | | | | | | |

Table 131. shows 0% mouse SOD1 mRNA remaining relative to PBS control in the diaphragm 14 days post dosing. N=5. N.D.: Not determined.

| | animal No. | | | | | |
|---|---|---|---|---|---|---|
| siRNA | 1 | 2 | 3 | 4 | 5 | Mean |
| PBS | 144.92 | 99.33 | 101.93 | 77.94 | 75.89 | 100.00 |
| DSR- | 71.24 | 109.36 | 91.45 | 90.45 | 89.24 | 90.35 |
| 0103301 | | | | | | |
| DSR- | 72.45 | 74.99 | 64.46 | 53.97 | 44.24 | 62.02 |
| 0103966 | | | | | | |
| DSR- | 51.69 | 49.49 | 45.69 | 61.79 | 54.62 | 52.66 |
| 0103967 | | | | | | |
| DSR- | 46.83 | 44.75 | 35.78 | 40.42 | 31.43 | 39.84 |
| 0103968 | | | | | | |
| DSR- | 41.18 | 66.70 | 23.14 | 33.42 | 50.21 | 42.93 |
| 0103969 | | | | | | |
| DSR- | 51.31 | 56.94 | 40.89 | 50.61 | 47.58 | 49.47 |
| 0103970 | | | | | | |
| DSR- | 46.69 | 45.35 | 44.82 | 47.79 | 53.08 | 47.54 |
| 0103971 | | | | | | |
| DSR- | 32.06 | 32.69 | 50.27 | 39.15 | 31.33 | 37.10 |
| 0103972 | | | | | | |
| DSR- | 61.49 | 82.46 | 64.69 | 55.92 | 55.05 | 63.92 |
| 0103973 | | | | | | |

Table 132. shows 00 mouse SOD1 mRNA remaining relative to PBS control in the kidney 14 days post dosing. N=5. N.D.: Not determined.

| | animal No. | | | | | |
|---|---|---|---|---|---|---|
| siRNA | 1 | 2 | 3 | 4 | 5 | Mean |
| PBS | 74.03 | 135.48 | 74.55 | 125.76 | 90.18 | 100.00 |
| DSR- | 153.76 | 117.75 | 160.86 | 141.15 | 157.42 | 146.19 |
| 0103301 | | | | | | |
| DSR- | 64.17 | 46.23 | 51.26 | 51.97 | 56.40 | 54.01 |
| 0103966 | | | | | | |
| DSR- | 72.54 | 72.16 | 73.68 | 55.02 | 65.81 | 67.84 |
| 0103967 | | | | | | |
| DSR- | 48.32 | 55.39 | 81.81 | 70.76 | 45.83 | 60.42 |
| 0103968 | | | | | | |
| DSR- | 48.05 | 67.41 | 46.72 | 46.74 | 65.31 | 54.85 |
| 0103969 | | | | | | |
| DSR- | 39.30 | 39.37 | 31.29 | 28.99 | 28.17 | 33.42 |
| 0103970 | | | | | | |
| DSR- | 48.38 | 54.30 | 47.44 | 47.42 | 48.37 | 49.18 |
| 0103971 | | | | | | |
| DSR- | 45.75 | 48.74 | 49.47 | 44.88 | 40.72 | 45.91 |
| 0103972 | | | | | | |
| DSR- | 56.97 | 58.43 | 62.70 | 58.76 | 62.54 | 59.88 |
| 0103973 | | | | | | |

Table 133. shows % mouse SOD1 mRNA remaining relative to PBS control in the heart 14 days post dosing. N=5. N.D.: Not determined.

| | animal No. | | | | | |
|---|---|---|---|---|---|---|
| siRNA | 1 | 2 | 3 | 4 | 5 | Mean |
| PBS | 91.79 | 89.69 | 117.44 | 96.54 | 104.55 | 100.00 |
| DSR- | 88.86 | 89.56 | 86.37 | 54.16 | 57.16 | 75.22 |
| 0103301 | | | | | | |
| DSR- | 83.51 | 86.09 | 75.36 | 93.07 | 92.15 | 86.04 |
| 0103966 | | | | | | |
| DSR- | 62.10 | 67.18 | 53.06 | 60.65 | 48.43 | 58.28 |
| 0103967 | | | | | | |
| DSR- | 46.27 | 61.16 | 48.35 | 48.72 | 33.29 | 47.56 |
| 0103968 | | | | | | |
| DSR- | 39.43 | 28.22 | 28.00 | 30.86 | 52.83 | 35.87 |
| 0103969 | | | | | | |
| DSR- | 59.02 | 50.14 | 43.80 | 47.55 | 48.31 | 49.76 |
| 0103970 | | | | | | |
| DSR- | 44.01 | 52.12 | 54.20 | 43.53 | 42.61 | 47.30 |
| 0103971 | | | | | | |
| DSR- | 40.97 | 41.53 | 41.05 | 33.81 | 34.29 | 38.33 |
| 0103972 | | | | | | |
| DSR- | 49.31 | 47.09 | 43.57 | 40.82 | 54.54 | 47.07 |
| 0103973 | | | | | | |

Table 134. shows % mouse SOD1 mRNA remaining relative to PBS control in the brown adipose tissue 14 days post dosing. N=5. N.D.: Not determined.

| | animal No. | | | | | |
|---|---|---|---|---|---|---|
| siRNA | 1 | 2 | 3 | 4 | 5 | Mean |
| PBS | 115.33 | 61.22 | 110.89 | 96.12 | 116.45 | 100.00 |
| DSR- | 87.99 | 107.63 | 100.08 | 95.56 | 84.66 | 95.18 |
| 0103301 | | | | | | |
| DSR- | 45.56 | 75.35 | 81.71 | 89.80 | 85.48 | 75.58 |
| 0103966 | | | | | | |
| DSR- | 66.17 | 19.37 | 52.57 | 60.09 | 44.20 | 48.48 |
| 0103967 | | | | | | |
| DSR- | 34.38 | 77.87 | 49.98 | 39.96 | 30.47 | 46.53 |
| 0103968 | | | | | | |
| DSR- | 22.80 | 30.54 | 28.58 | 33.18 | 41.67 | 31.35 |
| 0103969 | | | | | | |
| DSR- | 66.32 | 68.81 | 72.18 | 61.66 | 60.39 | 65.87 |
| 0103970 | | | | | | |
| DSR- | 45.50 | 77.53 | 70.70 | 48.28 | 50.06 | 58.41 |
| 0103971 | | | | | | |
| DSR- | 64.15 | 35.70 | 37.35 | 34.49 | 25.57 | 39.45 |
| 0103972 | | | | | | |
| DSR- | 17.71 | 18.62 | 14.44 | 19.55 | 16.60 | 17.38 |
| 0103973 | | | | | | |

Table 135. shows 0 mouse SOD1 mRNA remaining relative to PBS control in the sciatic nerve 14 days post dosing. N=5. N.D.: Not determined.

| | animal No. | | | | | |
|---|---|---|---|---|---|---|
| siRNA | 1 | 2 | 3 | 4 | 5 | Mean |
| PBS | 107.08 | 118.32 | 70.75 | 84.99 | 118.85 | 100.00 |
| DSR- | 64.26 | 53.99 | 54.35 | 74.62 | 58.95 | 61.23 |
| 0103301 | | | | | | |
| DSR- | 77.13 | 61.46 | 73.76 | 78.31 | 44.48 | 67.03 |
| 0103966 | | | | | | |
| DSR- | 52.63 | 87.92 | 88.42 | 48.29 | 123.15 | 80.08 |
| 0103967 | | | | | | |
| DSR- | 70.46 | 84.33 | 92.46 | 84.62 | 114.57 | 89.29 |
| 0103968 | | | | | | |
| DSR- | 72.02 | 72.06 | 83.91 | 74.07 | 93.48 | 79.11 |
| 0103969 | | | | | | |
| DSR- | 66.91 | 79.45 | 158.02 | 77.31 | 70.56 | 90.45 |
| 0103970 | | | | | | |
| DSR- | 58.28 | 84.77 | 81.38 | 82.78 | 66.61 | 74.77 |
| 0103971 | | | | | | |
| DSR- | 90.88 | 90.53 | 84.72 | 112.18 | 111.85 | 98.03 |
| 0103972 | | | | | | |
| DSR- | 93.07 | 87.41 | 71.50 | 126.02 | 93.91 | 94.38 |
| 0103973 | | | | | | |

Table 136. shows % mouse SOD1 mRNA remaining relative to PBS control in the kidney 28 days post dosing. N=5. N.D.: Not determined.

| | animal No. | | | | | |
|---|---|---|---|---|---|---|
| siRNA | 1 | 2 | 3 | 4 | 5 | Mean |
| PBS | 84.31 | 109.05 | 83.68 | 86.11 | 136.86 | 100.00 |
| DSR- | 108.99 | 80.43 | 89.94 | 76.17 | 77.62 | 86.63 |
| 0103301 | | | | | | |
| DSR- | 71.84 | 36.95 | 33.35 | 45.02 | 48.13 | 47.06 |
| 0103966 | | | | | | |
| DSR- | 57.45 | 47.72 | 61.47 | 52.38 | 44.79 | 52.76 |
| 0103967 | | | | | | |
| DSR- | 86.61 | 58.64 | 51.98 | 84.41 | 132.57 | 82.84 |
| 0103968 | | | | | | |
| DSR- | 35.54 | 46.64 | 52.21 | 48.39 | 48.21 | 46.20 |
| 0103969 | | | | | | |
| DSR- | 31.27 | 39.31 | 29.94 | 27.64 | 48.00 | 35.23 |
| 0103970 | | | | | | |
| DSR- | 32.86 | 41.89 | 38.76 | 45.97 | 38.74 | 39.64 |
| 0103971 | | | | | | |
| DSR- | 33.42 | 43.23 | 44.58 | 39.15 | 36.90 | 39.45 |
| 0103972 | | | | | | |
| DSR- | 46.37 | 64.79 | 103.57 | 42.23 | 50.00 | 61.39 |
| 0103973 | | | | | | |

Table 137. shows % mouse SOD1 mRNA remaining relative to PBS control in the liver 28 days post dosing. N=5. N.D.: Not determined.

| | animal No. | | | | | |
|---|---|---|---|---|---|---|
| siRNA | 1 | 2 | 3 | 4 | 5 | Mean |
| PBS | 144.45 | 85.98 | 122.11 | 79.00 | 68.47 | 100.00 |
| DSR- | 100.54 | 73.36 | 48.72 | 68.00 | 36.30 | 65.39 |
| 0103301 | | | | | | |
| DSR- | 95.02 | 68.33 | 51.90 | 33.43 | 28.66 | 55.47 |
| 0103966 | | | | | | |
| DSR- | 43.88 | N.D | 47.76 | 29.06 | 34.07 | 30.95 |
| 0103967 | | | | | | |
| DSR- | 48.97 | 51.65 | 42.01 | 29.62 | 62.28 | 46.91 |
| 0103968 | | | | | | |
| DSR- | 64.86 | 28.88 | 43.79 | 58.65 | 29.79 | 45.19 |
| 0103969 | | | | | | |
| DSR- | 105.47 | 63.17 | 36.19 | 47.94 | 113.20 | 73.20 |
| 0103970 | | | | | | |
| DSR- | 36.45 | 48.36 | 36.05 | 69.46 | 36.58 | 45.38 |
| 0103971 | | | | | | |
| DSR- | 16.03 | 23.57 | 15.49 | 33.22 | N.D | 17.66 |
| 0103972 | | | | | | |
| DSR- | 29.89 | 28.62 | 31.19 | 35.31 | 25.30 | 30.06 |
| 0103973 | | | | | | |

Table 138. shows % mouse SOD1 mRNA remaining relative to PBS control in the lung 28 days post dosing. N=5. N.D.: Not determined.

| | animal No. | | | | | |
|---|---|---|---|---|---|---|
| siRNA | 1 | 2 | 3 | 4 | 5 | Mean |
| PBS | 131.58 | 91.47 | 110.52 | 84.16 | 82.27 | 100.00 |
| DSR- | 123.81 | 118.74 | 120.00 | 103.64 | 119.99 | 117.24 |
| 0103301 | | | | | | |
| DSR- | 98.33 | 98.60 | 115.33 | 66.64 | 83.48 | 92.48 |
| 0103966 | | | | | | |
| DSR- | 69.92 | 84.41 | 94.34 | 118.71 | 66.82 | 86.84 |
| 0103967 | | | | | | |
| DSR- | 131.02 | 72.71 | 71.77 | 73.20 | 132.83 | 96.31 |
| 0103968 | | | | | | |
| DSR- | 131.78 | 113.43 | 91.18 | 91.31 | 60.40 | 97.62 |
| 0103969 | | | | | | |
| DSR- | 186.84 | 155.21 | 85.99 | 141.00 | 91.62 | 132.13 |
| 0103970 | | | | | | |
| DSR- | 279.94 | 220.40 | 174.03 | 173.51 | 151.51 | 199.88 |
| 0103971 | | | | | | |
| DSR- | 109.99 | 108.93 | 91.98 | 100.72 | 100.21 | 102.37 |
| 0103972 | | | | | | |
| DSR- | 88.64 | 69.07 | 85.09 | 70.41 | 94.20 | 81.48 |
| 0103973 | | | | | | |

Table 139. shows % mouse SOD1 mRNA remaining relative to PBS control in the white adipose tissue 28 days post dosing. N=5. N.D.: Not determined.

| | animal No. | | | | | |
|---|---|---|---|---|---|---|
| siRNA | 1 | 2 | 3 | 4 | 5 | Mean |
| PBS | 94.07 | 102.55 | 68.52 | 117.52 | 117.34 | 100.00 |
| DSR- | 125.24 | 170.45 | 112.01 | 186.45 | 173.61 | 153.55 |
| 0103301 | | | | | | |
| DSR- | 82.01 | 149.20 | 109.65 | 91.63 | 110.98 | 108.70 |
| 0103966 | | | | | | |
| DSR- | 91.09 | 93.73 | 87.28 | 53.31 | 81.97 | 81.48 |
| 0103967 | | | | | | |
| DSR- | 59.79 | 59.79 | 51.43 | 63.19 | 59.41 | 58.72 |
| 0103968 | | | | | | |
| DSR- | 28.52 | 99.87 | 26.45 | 29.90 | 25.19 | 41.98 |
| 0103969 | | | | | | |
| DSR- | 87.01 | 119.88 | 103.78 | 91.24 | 153.19 | 111.02 |
| 0103970 | | | | | | |
| DSR- | 79.54 | 112.65 | 100.26 | 66.54 | 98.24 | 91.45 |
| 0103971 | | | | | | |
| DSR- | 14.19 | 44.21 | 34.07 | 31.42 | 48.54 | 34.49 |
| 0103972 | | | | | | |
| DSR- | 87.60 | 93.56 | 99.00 | 131.83 | 118.41 | 106.08 |
| 0103973 | | | | | | |

Table 140. shows % mouse SOD1 mRNA remaining relative to PBS control in the brown adipose tissue 28 days post dosing. N=5. N.D.: Not determined.

| | animal No. | | | | | |
|---|---|---|---|---|---|---|
| siRNA | 1 | 2 | 3 | 4 | 5 | Mean |
| PBS | 106.11 | 93.51 | 85.87 | 110.31 | 104.20 | 100.00 |
| DSR- | 119.27 | 114.23 | 136.73 | 113.12 | 110.21 | 118.71 |
| 0103301 | | | | | | |
| DSR- | 76.10 | 81.39 | 54.23 | 68.75 | 64.02 | 68.90 |
| 0103966 | | | | | | |
| DSR- | 49.69 | 57.20 | 77.69 | 47.15 | 42.89 | 54.92 |
| 0103967 | | | | | | |
| DSR- | 46.00 | 47.95 | 34.80 | 39.24 | 55.35 | 44.67 |
| 0103968 | | | | | | |
| DSR- | 41.64 | 29.27 | 31.11 | 33.02 | 38.21 | 34.65 |
| 0103969 | | | | | | |
| DSR- | 90.01 | 74.38 | 67.29 | 52.89 | 56.31 | 68.17 |
| 0103970 | | | | | | |
| DSR- | 23.77 | 116.00 | 59.02 | 56.81 | 69.54 | 65.03 |
| 0103971 | | | | | | |
| DSR- | 38.11 | 50.34 | 37.78 | 31.89 | 46.08 | 40.84 |
| 0103972 | | | | | | |
| DSR- | 34.82 | 56.04 | 45.44 | 58.40 | 38.53 | 46.65 |
| 0103973 | | | | | | |

Table 141. shows % mouse SOD1 mRNA remaining relative to PBS control in the quad 28 days post dosing. N=5. N.D.: Not determined.

| | animal No. | | | | | |
|---|---|---|---|---|---|---|
| siRNA | 1 | 2 | 3 | 4 | 5 | Mean |
| PBS | 88.66 | 120.42 | 103.49 | 81.45 | 105.98 | 100.00 |
| DSR- | 96.11 | 114.54 | 102.34 | 121.09 | 104.74 | 107.76 |
| 0103301 | | | | | | |
| DSR- | 79.85 | 76.33 | 82.12 | 73.40 | 74.68 | 77.28 |
| 0103966 | | | | | | |
| DSR- | 73.59 | 75.45 | 57.53 | 71.99 | 63.18 | 68.35 |
| 0103967 | | | | | | |
| DSR- | 68.02 | 69.04 | 52.62 | 64.34 | 65.86 | 63.97 |
| 0103968 | | | | | | |
| DSR- | 56.00 | 61.96 | 50.33 | 50.52 | 50.30 | 53.82 |
| 0103969 | | | | | | |
| DSR- | 70.38 | 70.61 | 65.62 | 71.25 | 63.91 | 68.35 |
| 0103970 | | | | | | |
| DSR- | 66.26 | 68.94 | 68.27 | 64.51 | 59.40 | 65.48 |
| 0103971 | | | | | | |
| DSR- | 71.05 | 74.03 | 72.04 | 71.38 | 67.96 | 71.29 |
| 0103972 | | | | | | |
| DSR- | 97.33 | 96.61 | 86.43 | 100.79 | 85.05 | 93.24 |
| 0103973 | | | | | | |

Table 142. shows % mouse SOD1 mRNA remaining relative to PBS control in the diaphragm 28 days post dosing. N=5. N.D.: Not determined.

| | animal No. | | | | | |
|---|---|---|---|---|---|---|
| siRNA | 1 | 2 | 3 | 4 | 5 | Mean |
| PBS | 83.26 | 112.06 | 78.72 | 92.79 | 133.17 | 100.00 |
| DSR- | 62.33 | 76.97 | 70.93 | 78.78 | 100.03 | 77.81 |
| 0103301 | | | | | | |
| DSR- | 59.61 | 77.34 | 70.65 | 84.77 | 65.53 | 71.58 |
| 0103966 | | | | | | |
| DSR- | 87.58 | 64.29 | 58.83 | 57.11 | 66.80 | 66.92 |
| 0103967 | | | | | | |
| DSR- | 60.23 | 61.95 | 49.54 | 47.79 | 53.74 | 54.65 |
| 0103968 | | | | | | |
| DSR- | 49.18 | 62.02 | 69.92 | 48.91 | 46.98 | 55.40 |
| 0103969 | | | | | | |
| DSR- | 68.88 | 62.33 | 112.39 | 109.78 | 89.10 | 88.50 |
| 0103970 | | | | | | |
| DSR- | 58.38 | 78.39 | 84.39 | 76.11 | 73.13 | 74.08 |
| 0103971 | | | | | | |
| DSR- | 31.36 | 39.44 | 49.05 | 43.40 | 37.05 | 40.06 |
| 0103972 | | | | | | |
| DSR- | 74.04 | 76.44 | 71.23 | 75.46 | 80.24 | 75.48 |
| 0103973 | | | | | | |

Table 143. shows % mouse SOD1 mRNA remaining relative to PBS control in the nerve 28 days post dosing. N=5. N.D.: Not determined.

| | animal No. | | | | | |
|---|---|---|---|---|---|---|
| siRNA | 1 | 2 | 3 | 4 | 5 | Mean |
| PBS | 114.45 | 101.39 | 109.73 | 71.19 | 103.24 | 100.00 |
| DSR- | 140.20 | 49.68 | 94.50 | 124.83 | 160.04 | 113.85 |
| 0103301 | | | | | | |
| DSR- | 61.10 | 87.58 | 82.39 | 82.94 | 103.81 | 83.56 |
| 0103966 | | | | | | |
| DSR- | 176.57 | 101.67 | 108.07 | 83.77 | 137.56 | 121.53 |
| 0103967 | | | | | | |
| DSR- | 66.54 | 114.77 | 81.96 | 61.68 | 95.10 | 84.01 |
| 0103968 | | | | | | |
| DSR- | 39.54 | 64.27 | 66.82 | 128.64 | 104.96 | 80.85 |
| 0103969 | | | | | | |
| DSR- | 116.41 | 82.85 | 74.08 | 54.17 | 48.26 | 75.15 |
| 0103970 | | | | | | |
| DSR- | 117.84 | 103.90 | 67.49 | 117.58 | 84.41 | 98.24 |
| 0103971 | | | | | | |
| DSR- | 61.89 | 59.78 | 58.70 | 151.07 | 87.68 | 83.82 |
| 0103972 | | | | | | |
| DSR- | 114.05 | 214.60 | 87.80 | 285.22 | 132.29 | 166.79 |
| 0103973 | | | | | | |

Table 144. shows % mouse SOD1 mRNA remaining relative to PBS control in the heart 28 days post dosing. N=5. N.D.: Not determined.

| | animal No. | | | | | |
|---|---|---|---|---|---|---|
| siRNA | 1 | 2 | 3 | 4 | 5 | Mean |
| PBS | 166.90 | 69.60 | 70.84 | 68.86 | 123.80 | 100.00 |
| DSR- | 153.35 | 77.87 | 138.07 | 124.13 | 133.26 | 125.34 |
| 0103301 | | | | | | |
| DSR- | 141.58 | 118.97 | 64.38 | 103.39 | 84.04 | 102.47 |
| 0103966 | | | | | | |
| DSR- | 90.96 | 58.25 | 90.06 | 111.57 | 108.65 | 91.90 |
| 0103967 | | | | | | |
| DSR- | 103.28 | 43.46 | 64.24 | 39.05 | 40.87 | 58.18 |
| 0103968 | | | | | | |
| DSR- | 47.62 | 71.48 | 42.58 | 39.95 | 45.10 | 49.35 |
| 0103969 | | | | | | |
| DSR- | 66.54 | 64.13 | 64.72 | 56.31 | 57.79 | 61.90 |
| 0103970 | | | | | | |
| DSR- | 75.70 | 76.86 | 66.89 | 62.33 | 71.58 | 70.67 |
| 0103971 | | | | | | |
| DSR- | 48.01 | 57.20 | 51.45 | 44.47 | 46.92 | 49.61 |
| 0103972 | | | | | | |
| DSR- | 82.65 | 84.59 | 85.51 | 80.80 | 83.11 | 83.33 |
| 0103973 | | | | | | |

Table 145. shows the accumulation of antisense strand in the liver at 14 days and 28 days post dosing. N=5. N.D.: Not determined.

| | | animal No. | | | | | |
|---|---|---|---|---|---|---|---|
| | siRNA | 1 | 2 | 3 | 4 | 5 | Mean |
| 14 days | PBS | 0.00 | 0.00 | 0.00 | 0.00 | 0.00 | 0.00 |
| post | DSR- | 5.60 | 5.49 | 6.11 | N.D | 6.18 | 4.68 |
| dosing | 0103301 | | | | | | |
| | DSR- | 4.44 | 4.88 | 3.85 | 3.58 | 4.18 | 4.19 |
| | 0103966 | | | | | | |
| | DSR- | 6.12 | 8.59 | 7.32 | 7.15 | 7.77 | 7.39 |
| | 0103967 | | | | | | |
| | DSR- | 7.24 | 6.96 | 7.27 | 6.59 | 6.99 | 7.01 |
| | 0103968 | | | | | | |
| | DSR- | 6.48 | 6.65 | 7.15 | 6.56 | 8.35 | 7.03 |
| | 0103969 | | | | | | |
| | DSR- | 4.50 | 3.34 | 4.15 | 4.38 | 4.40 | 4.15 |
| | 0103970 | | | | | | |
| | DSR- | 6.69 | 6.67 | 6.88 | 6.51 | 8.64 | 7.08 |
| | 0103971 | | | | | | |
| | DSR- | 5.22 | 8.51 | 6.43 | 7.44 | 7.91 | 7.10 |
| | 0103972 | | | | | | |
| | DSR- | 5.64 | 6.13 | 5.04 | 5.94 | 4.99 | 5.55 |
| | 0103973 | | | | | | |
| 28 days | PBS | 0.00 | 0.00 | 0.00 | 0.00 | 0.00 | 0.00 |
| post | DSR- | 3.04 | 3.72 | 2.75 | 2.98 | 3.29 | 3.16 |
| dosing | 0103301 | | | | | | |
| | DSR- | 2.65 | 1.77 | 2.47 | 3.28 | 3.11 | 2.66 |
| | 0103966 | | | | | | |
| | DSR- | 3.91 | 4.31 | 4.17 | 4.66 | 5.28 | 4.47 |
| | 0103967 | | | | | | |
| | DSR- | 5.06 | 5.65 | 4.35 | 5.51 | 5.98 | 5.31 |
| | 0103968 | | | | | | |
| | DSR- | 4.51 | 5.99 | 4.92 | 4.94 | 7.19 | 5.51 |
| | 0103969 | | | | | | |
| | DSR- | 2.38 | 1.92 | 2.25 | 2.95 | 1.42 | 2.19 |
| | 0103970 | | | | | | |
| | DSR- | 4.53 | 5.84 | 4.77 | 6.31 | 8.37 | 5.96 |
| | 0103971 | | | | | | |
| | DSR- | 4.62 | 5.71 | 3.27 | 5.49 | 5.97 | 5.01 |
| | 0103972 | | | | | | |
| | DSR- | 1.90 | 2.82 | 3.73 | 2.94 | 6.15 | 3.51 |
| | 0103973 | | | | | | |

Table 146. shows the accumulation of antisense strand in the kidney at 14 days and 28 days post dosing. N=5. N.D.: Not determined.

| | | animal No. | | | | | |
|---|---|---|---|---|---|---|---|
| | siRNA | 1 | 2 | 3 | 4 | 5 | Mean |
| 14 days | PBS | 0.00 | 0.00 | 0.00 | 0.00 | 0.00 | 0.00 |
| post | DSR- | 3.47 | 4.22 | 3.91 | N.D | 4.56 | 3.23 |
| dosing | 0103301 | | | | | | |
| | DSR- | 7.71 | 7.52 | 7.67 | 8.30 | 7.75 | 7.79 |
| | 0103966 | | | | | | |
| | DSR- | 7.58 | 7.55 | 8.65 | 10.77 | 13.72 | 9.65 |
| | 0103967 | | | | | | |
| | DSR- | 1.81 | 1.57 | 2.62 | 2.09 | 2.73 | 2.17 |
| | 0103968 | | | | | | |
| | DSR- | 4.69 | 4.27 | 5.90 | 3.52 | 4.00 | 4.48 |
| | 0103969 | | | | | | |
| | DSR- | 7.35 | 6.96 | 11.60 | 18.41 | 13.33 | 11.53 |
| | 0103970 | | | | | | |
| | DSR- | 9.70 | 12.42 | 13.25 | 13.97 | 12.49 | 12.37 |
| | 0103971 | | | | | | |
| | DSR- | 1.80 | 3.35 | 2.09 | 2.48 | 2.99 | 2.54 |
| | 0103972 | | | | | | |
| | DSR- | 0.36 | 0.40 | 0.39 | 0.33 | 0.35 | 0.37 |
| | 0103973 | | | | | | |
| 28 days | PBS | 0.00 | 0.00 | 0.00 | 0.00 | 0.00 | 0.00 |
| post | DSR- | 2.40 | 2.20 | 2.35 | 2.03 | 2.17 | 2.23 |
| dosing | 0103301 | | | | | | |
| | DSR- | 3.47 | 6.05 | 5.40 | 4.99 | 4.09 | 4.80 |
| | 0103966 | | | | | | |
| | DSR- | 5.33 | 5.17 | 7.44 | 10.66 | 8.02 | 7.32 |
| | 0103967 | | | | | | |
| | DSR- | 1.84 | 2.16 | 2.05 | 2.06 | 2.39 | 2.10 |
| | 0103968 | | | | | | |
| | DSR- | 3.00 | 2.71 | 4.55 | 3.89 | 3.94 | 3.62 |
| | 0103969 | | | | | | |
| | DSR- | 3.88 | 4.35 | 3.84 | 3.72 | 4.89 | 4.14 |
| | 0103970 | | | | | | |
| | DSR- | 3.57 | 3.71 | 3.57 | 4.55 | 4.43 | 3.97 |
| | 0103971 | | | | | | |
| | DSR- | 2.04 | 1.32 | 1.62 | 1.86 | 1.66 | 1.70 |
| | 0103972 | | | | | | |
| | DSR- | 0.34 | 0.41 | 0.48 | 0.50 | 0.59 | 0.46 |
| | 0103973 | | | | | | |

Table 147. shows the accumulation of antisense strand in the lung at 14 days and 28 days post dosing. N=5. N.D.: Not determined.

| | | animal No. | | | | | |
|---|---|---|---|---|---|---|---|
| | siRNA | 1 | 2 | 3 | 4 | 5 | Mean |
| 14 days | PBS | 0.00 | 0.00 | 0.00 | 0.00 | 0.00 | 0.00 |
| post | DSR- | 0.13 | 0.22 | 0.19 | 0.01 | 0.16 | 0.14 |
| dosing | 0103301 | | | | | | |
| | DSR- | 0.37 | 0.36 | 0.14 | 0.20 | 0.21 | 0.26 |
| | 0103966 | | | | | | |
| | DSR- | 0.24 | 0.36 | 0.46 | 0.45 | 0.35 | 0.37 |
| | 0103967 | | | | | | |
| | DSR- | 0.22 | 0.17 | 0.20 | 0.26 | 0.20 | 0.21 |
| | 0103968 | | | | | | |
| | DSR- | 0.37 | 0.50 | 0.60 | 0.30 | 0.42 | 0.44 |
| | 0103969 | | | | | | |
| | DSR- | 0.27 | 0.24 | 0.22 | 0.27 | 0.33 | 0.27 |
| | 0103970 | | | | | | |
| | DSR- | 0.24 | 0.33 | 0.39 | 0.30 | 0.25 | 0.30 |
| | 0103971 | | | | | | |
| | DSR- | 0.28 | 0.35 | 0.32 | 0.31 | 0.36 | 0.32 |
| | 0103972 | | | | | | |
| | DSR- | 0.10 | 0.17 | 0.07 | 0.19 | 0.10 | 0.13 |
| | 0103973 | | | | | | |
| 28 days | PBS | 0.00 | 0.00 | 0.00 | 0.00 | 0.00 | 0.00 |
| post | DSR- | 0.16 | 0.13 | 0.07 | 0.07 | 0.13 | 0.11 |
| dosing | 0103301 | | | | | | |
| | DSR- | 0.08 | 0.10 | 0.10 | 0.10 | 0.09 | 0.09 |
| | 0103966 | | | | | | |
| | DSR- | 0.11 | 0.15 | 0.20 | 0.13 | 0.20 | 0.16 |
| | 0103967 | | | | | | |
| | DSR- | 0.15 | 0.15 | 0.15 | 0.21 | 0.20 | 0.17 |
| | 0103968 | | | | | | |
| | DSR- | 0.27 | 0.24 | 0.35 | 0.39 | 0.32 | 0.31 |
| | 0103969 | | | | | | |
| | DSR- | 0.07 | 0.09 | 0.09 | 0.07 | 0.14 | 0.09 |
| | 0103970 | | | | | | |
| | DSR- | 0.14 | 0.12 | 0.18 | 0.17 | 0.16 | 0.15 |
| | 0103971 | | | | | | |
| | DSR- | 0.18 | 0.08 | 0.14 | 0.17 | 0.16 | 0.15 |
| | 0103972 | | | | | | |
| | DSR- | 0.05 | 0.05 | 0.04 | 0.04 | 0.07 | 0.05 |
| | 0103973 | | | | | | |

Table 148. shows the accumulation of antisense strand in the heart at 14 days and 28 days post dosing. N=5. N.D.: Not determined.

| | | animal No. | | | | | |
|---|---|---|---|---|---|---|---|
| | siRNA | 1 | 2 | 3 | 4 | 5 | Mean |
| 14 days | PBS | 0.00 | 0.00 | 0.00 | 0.00 | 0.00 | 0.00 |
| post | DSR- | 0.30 | 0.46 | 0.37 | N.D | 0.34 | 0.30 |
| dosing | 0103301 | | | | | | |
| | DSR- | 0.20 | 0.23 | 0.24 | 0.20 | 0.21 | 0.20 |
| | 0103966 | | | | | | |
| | DSR- | 0.17 | 0.24 | 0.25 | 0.28 | 0.32 | 0.17 |
| | 0103967 | | | | | | |
| | DSR- | 0.20 | 0.16 | 0.33 | 0.21 | 0.27 | 0.20 |
| | 0103968 | | | | | | |
| | DSR- | 0.34 | 0.29 | 0.37 | 0.25 | 0.26 | 0.34 |
| | 0103969 | | | | | | |
| | DSR- | 0.33 | 0.30 | 0.35 | 0.50 | 0.31 | 0.33 |
| | 0103970 | | | | | | |
| | DSR- | 0.50 | 0.44 | 0.67 | 0.48 | 0.40 | 0.50 |
| | 0103971 | | | | | | |
| | DSR- | 0.28 | 0.37 | 0.35 | 0.26 | 0.38 | 0.28 |
| | 0103972 | | | | | | |
| | DSR- | 0.07 | 0.09 | 0.06 | 0.09 | 0.08 | 0.07 |
| | 0103973 | | | | | | |
| 28 days | PBS | 0.00 | 0.00 | 0.00 | 0.00 | 0.00 | 0.00 |
| post | DSR- | 0.23 | 0.22 | 0.19 | 0.21 | 0.24 | 0.22 |
| dosing | 0103301 | | | | | | |
| | DSR- | 0.16 | 0.25 | 0.26 | 0.18 | 0.21 | 0.21 |
| | 0103966 | | | | | | |
| | DSR- | 0.22 | 0.23 | 0.27 | 0.27 | 0.26 | 0.25 |
| | 0103967 | | | | | | |
| | DSR- | 0.27 | 0.23 | 0.19 | 0.37 | 0.37 | 0.29 |
| | 0103968 | | | | | | |
| | DSR- | 0.26 | 0.22 | 0.25 | 0.28 | 0.46 | 0.29 |
| | 0103969 | | | | | | |
| | DSR- | 0.18 | 0.11 | 0.16 | 0.19 | 0.18 | 0.16 |
| | 0103970 | | | | | | |
| | DSR- | 0.23 | 0.17 | 0.25 | 0.31 | 0.30 | 0.25 |
| | 0103971 | | | | | | |
| | DSR- | 0.21 | 0.21 | 0.32 | 0.24 | 0.25 | 0.25 |
| | 0103972 | | | | | | |
| | DSR- | 0.03 | 0.04 | 0.04 | 0.04 | 0.14 | 0.06 |
| | 0103973 | | | | | | |

Table 149. shows the accumulation of antisense strand in the diaphragm at 14 days and 28 days post dosing. N=5. N.D.: Not determined.

| | | animal No. | | | | | |
|---|---|---|---|---|---|---|---|
| | siRNA | 1 | 2 | 3 | 4 | 5 | Mean |
| 14 days | PBS | 0.00 | 0.00 | 0.00 | 0.00 | 0.00 | 0.00 |
| post | DSR- | 0.17 | 0.28 | 0.19 | N.D | 0.19 | 0.17 |
| dosing | 0103301 | | | | | | |
| | DSR- | 0.34 | 0.23 | 0.14 | 0.13 | 0.15 | 0.20 |
| | 0103966 | | | | | | |
| | DSR- | 0.63 | 0.83 | 0.57 | 2.23 | 0.35 | 0.92 |
| | 0103967 | | | | | | |
| | DSR- | 0.15 | 0.09 | 0.16 | 0.10 | 0.16 | 0.13 |
| | 0103968 | | | | | | |
| | DSR- | 0.34 | 0.29 | 1.15 | 0.15 | 0.45 | 0.48 |
| | 0103969 | | | | | | |
| | DSR- | 0.16 | 0.11 | 0.22 | 0.21 | 0.18 | 0.18 |
| | 0103970 | | | | | | |
| | DSR- | 0.29 | 0.31 | 0.27 | 0.25 | 0.18 | 0.26 |
| | 0103971 | | | | | | |
| | DSR- | 0.18 | 0.19 | 0.17 | 0.19 | 0.17 | 0.18 |
| | 0103972 | | | | | | |
| | DSR- | 0.25 | 0.12 | 0.80 | 0.05 | 0.16 | 0.28 |
| | 0103973 | | | | | | |
| 28 days | PBS | 0.00 | 0.00 | 0.00 | 0.00 | 0.00 | 0.00 |
| post | DSR- | 0.08 | 0.06 | 0.07 | 0.07 | 0.10 | 0.07 |
| dosing | 0103301 | | | | | | |
| | DSR- | 0.10 | 0.13 | 0.11 | 0.19 | 0.12 | 0.13 |
| | 0103966 | | | | | | |
| | DSR- | 0.09 | 0.09 | 0.11 | 0.07 | 0.11 | 0.09 |
| | 0103967 | | | | | | |
| | DSR- | 0.14 | 0.07 | 0.06 | 0.14 | 0.10 | 0.10 |
| | 0103968 | | | | | | |
| | DSR- | 0.15 | 0.30 | 0.17 | 0.09 | 0.12 | 0.17 |
| | 0103969 | | | | | | |
| | DSR- | 0.07 | 0.11 | 0.10 | 0.56 | 0.10 | 0.19 |
| | 0103970 | | | | | | |
| | DSR- | 0.16 | 0.28 | 0.35 | 0.20 | 0.14 | 0.23 |
| | 0103971 | | | | | | |
| | DSR- | 0.33 | 0.11 | 0.13 | 0.07 | 0.13 | 0.16 |
| | 0103972 | | | | | | |
| | DSR- | 0.02 | 0.02 | 0.02 | 0.02 | 0.03 | 0.02 |
| | 0103973 | | | | | | |

Table 150. shows the accumulation of antisense strand in the quad at 14 days and 28 days post dosing. N=5. N.D.: Not determined.

| | | animal No. | | | | | |
|---|---|---|---|---|---|---|---|
| | siRNA | 1 | 2 | 3 | 4 | 5 | Mean |
| 14 days | PBS | 0.00 | 0.00 | 0.00 | 0.00 | 0.00 | 0.00 |
| post | DSR- | 0.12 | 0.25 | 0.11 | 0.01 | 0.14 | 0.13 |
| dosing | 0103301 | | | | | | |
| | DSR- | 0.13 | 0.15 | 0.21 | 0.10 | 0.10 | 0.14 |
| | 0103966 | | | | | | |
| | DSR- | 0.10 | 0.11 | 0.20 | 0.12 | 0.15 | 0.14 |
| | 0103967 | | | | | | |
| | DSR- | 0.08 | 0.05 | 0.06 | 0.06 | 0.07 | 0.06 |
| | 0103968 | | | | | | |
| | DSR- | 0.13 | 0.09 | 0.09 | 0.07 | 0.08 | 0.09 |
| | 0103969 | | | | | | |
| | DSR- | 0.13 | 0.10 | 0.17 | 0.17 | 0.11 | 0.14 |
| | 0103970 | | | | | | |
| | DSR- | 0.14 | 0.12 | 0.19 | 0.10 | 0.17 | 0.15 |
| | 0103971 | | | | | | |
| | DSR- | 0.07 | 0.09 | 0.05 | 0.09 | 0.60 | 0.18 |
| | 0103972 | | | | | | |
| | DSR- | 0.01 | 0.01 | 0.01 | 0.02 | 0.01 | 0.01 |
| | 0103973 | | | | | | |
| 28 days | PBS | 0.00 | 0.00 | 0.00 | 0.00 | 0.00 | 0.00 |
| post | DSR- | 0.09 | 0.06 | 0.05 | 0.06 | 0.07 | 0.06 |
| dosing | 0103301 | | | | | | |
| | DSR- | 0.12 | 0.09 | 0.11 | 0.12 | 0.07 | 0.10 |
| | 0103966 | | | | | | |
| | DSR- | 0.07 | 0.07 | 0.08 | 0.08 | 0.09 | 0.08 |
| | 0103967 | | | | | | |
| | DSR- | 0.04 | 0.04 | 0.05 | 0.06 | 0.13 | 0.06 |
| | 0103968 | | | | | | |
| | DSR- | 0.05 | 0.04 | 0.07 | 0.03 | 0.07 | 0.05 |
| | 0103969 | | | | | | |
| | DSR- | 0.09 | 0.11 | 0.15 | 0.14 | 0.22 | 0.14 |
| | 0103970 | | | | | | |
| | DSR- | 0.07 | 0.10 | 0.09 | 0.09 | 0.11 | 0.09 |
| | 0103971 | | | | | | |
| | DSR- | 0.10 | 0.05 | 0.03 | 0.05 | 0.09 | 0.07 |
| | 0103972 | | | | | | |
| | DSR- | 0.01 | 0.01 | 0.00 | 0.01 | 0.00 | 0.01 |
| | 0103973 | | | | | | |

Table 151. shows the accumulation of antisense strand in the white adipose at 14 days and 28 days post dosing. N=5. N.D.: Not determined.

| | | animal No. | | | | | |
|---|---|---|---|---|---|---|---|
| | siRNA | 1 | 2 | 3 | 4 | 5 | Mean |
| 14 days | PBS | 0.00 | 0.00 | 0.00 | 0.00 | 0.00 | 0.00 |
| post | DSR- | 0.06 | 0.11 | 0.09 | 0.01 | 0.10 | 0.07 |
| dosing | 0103301 | | | | | | |
| | DSR- | 0.08 | 0.10 | 0.07 | 0.05 | 0.04 | 0.07 |
| | 0103966 | | | | | | |
| | DSR- | 0.12 | 0.29 | 0.11 | 0.10 | 0.12 | 0.15 |
| | 0103967 | | | | | | |
| | DSR- | 0.10 | 0.06 | 0.12 | 0.07 | 0.08 | 0.09 |
| | 0103968 | | | | | | |
| | DSR- | 0.15 | 0.11 | 0.22 | 0.10 | 0.09 | 0.13 |
| | 0103969 | | | | | | |
| | DSR- | 0.13 | 0.08 | 0.08 | 0.14 | 0.10 | 0.11 |
| | 0103970 | | | | | | |
| | DSR- | 0.13 | 0.06 | 0.13 | 0.08 | 0.12 | 0.10 |
| | 0103971 | | | | | | |
| | DSR- | 0.15 | 0.12 | 0.09 | 0.10 | 0.11 | 0.11 |
| | 0103972 | | | | | | |
| | DSR- | 0.03 | 0.03 | 0.02 | 0.03 | 0.02 | 0.03 |
| | 0103973 | | | | | | |
| 28 days | PBS | 0.00 | 0.00 | 0.00 | 0.00 | 0.00 | 0.00 |
| post | DSR- | 0.03 | 0.02 | 0.02 | 0.02 | 0.04 | 0.03 |
| dosing | 0103301 | | | | | | |
| | DSR- | 0.02 | 0.02 | 0.03 | 0.02 | 0.02 | 0.02 |
| | 0103966 | | | | | | |
| | DSR- | 0.04 | 0.05 | 0.06 | 0.05 | 0.05 | 0.05 |
| | 0103967 | | | | | | |
| | DSR- | 0.07 | 0.07 | 0.04 | 0.09 | 0.05 | 0.06 |
| | 0103968 | | | | | | |
| | DSR- | 0.12 | 0.11 | 0.15 | 0.07 | 0.16 | 0.12 |
| | 0103969 | | | | | | |
| | DSR- | 0.02 | 0.02 | 0.02 | 0.04 | 0.04 | 0.03 |
| | 0103970 | | | | | | |
| | DSR- | 0.05 | 0.06 | 0.05 | 0.03 | 0.06 | 0.05 |
| | 0103971 | | | | | | |
| | DSR- | 0.07 | 0.05 | 0.10 | 0.06 | 0.08 | 0.07 |
| | 0103972 | | | | | | |
| | DSR- | 0.02 | 0.02 | 0.02 | 0.01 | 0.05 | 0.02 |
| | 0103973 | | | | | | |

Table 152. shows the accumulation of antisense strand in the brown adipose at 14 days and 28 days post dosing. N=5. N.D.: Not determined.

| | | animal No. | | | | | |
|---|---|---|---|---|---|---|---|
| | siRNA | 1 | 2 | 3 | 4 | 5 | Mean |
| 14 days | PBS | 0.00 | 0.00 | 0.00 | 0.00 | 0.00 | 0.00 |
| post | DSR- | 0.45 | 1.34 | 1.86 | 0.01 | 0.83 | 0.90 |
| dosing | 0103301 | | | | | | |
| | DSR- | 0.34 | 0.90 | 1.04 | 0.52 | 0.97 | 0.75 |
| | 0103966 | | | | | | |
| | DSR- | 1.02 | 0.38 | 1.62 | 1.61 | 1.31 | 1.19 |
| | 0103967 | | | | | | |
| | DSR- | 0.96 | 1.05 | 1.26 | 0.79 | 0.86 | 0.98 |
| | 0103968 | | | | | | |
| | DSR- | 1.99 | 1.72 | 1.51 | 0.40 | 0.29 | 1.18 |
| | 0103969 | | | | | | |
| | DSR- | 1.06 | 0.10 | 0.90 | 0.51 | 0.29 | 0.57 |
| | 0103970 | | | | | | |
| | DSR- | 0.91 | 0.97 | 2.84 | 1.03 | 1.72 | 1.49 |
| | 0103971 | | | | | | |
| | DSR- | 1.78 | 1.97 | 2.02 | 2.48 | 1.50 | 1.95 |
| | 0103972 | | | | | | |
| | DSR- | 2.98 | 4.58 | 4.06 | 2.80 | 2.79 | 3.44 |
| | 0103973 | | | | | | |
| 28 days | PBS | 0.00 | 0.00 | 0.00 | 0.00 | 0.00 | 0.00 |
| post | DSR- | 0.45 | 0.63 | 0.55 | 0.98 | 0.26 | 0.57 |
| dosing | 0103301 | | | | | | |
| | DSR- | 0.80 | 0.54 | 0.34 | 0.67 | 1.10 | 0.69 |
| | 0103966 | | | | | | |
| | DSR- | 0.62 | 0.49 | 0.96 | 1.96 | 1.13 | 1.03 |
| | 0103967 | | | | | | |
| | DSR- | 1.16 | 2.00 | 0.81 | 2.98 | 1.13 | 1.62 |
| | 0103968 | | | | | | |
| | DSR- | 1.50 | 2.66 | 5.19 | 2.11 | 1.55 | 2.60 |
| | 0103969 | | | | | | |
| | DSR- | 0.38 | 0.36 | 0.48 | 1.37 | 0.55 | 0.63 |
| | 0103970 | | | | | | |
| | DSR- | 0.68 | 0.12 | 1.19 | 0.67 | 1.07 | 0.75 |
| | 0103971 | | | | | | |
| | DSR- | 1.68 | 1.65 | 1.69 | 1.09 | 0.97 | 1.42 |
| | 0103972 | | | | | | |
| | DSR- | 2.18 | 1.81 | 2.14 | 2.29 | 2.89 | 2.26 |
| | 0103973 | | | | | | |

Table 153. shows the accumulation of antisense strand in the sciatic nerve at 14 days and 28 days post dosing. N=5. N.D.: Not determined.

| | | animal No. | | | | | |
|---|---|---|---|---|---|---|---|
| | siRNA | 1 | 2 | 3 | 4 | 5 | Mean |
| 14 days | PBS | 0.00 | 0.00 | 0.00 | 0.00 | 0.00 | 0.00 |
| post | DSR- | 0.06 | 0.08 | 0.10 | 0.00 | 0.09 | 0.07 |
| dosing | 0103301 | | | | | | |
| | DSR- | 0.10 | 0.05 | 0.05 | 0.06 | 0.04 | 0.06 |
| | 0103966 | | | | | | |
| | DSR- | 0.04 | 0.05 | 0.06 | 0.03 | 0.09 | 0.05 |
| | 0103967 | | | | | | |
| | DSR- | 0.02 | 0.02 | 0.00 | 0.01 | 0.05 | 0.02 |
| | 0103968 | | | | | | |
| | DSR- | 0.07 | 0.04 | 0.03 | 0.03 | 0.03 | 0.04 |
| | 0103969 | | | | | | |
| | DSR- | 0.09 | 0.06 | 0.12 | 0.05 | 0.10 | 0.09 |
| | 0103970 | | | | | | |
| | DSR- | 0.08 | 0.14 | 0.06 | 0.03 | 0.05 | 0.07 |
| | 0103971 | | | | | | |
| | DSR- | 0.03 | 0.03 | 0.01 | 0.03 | 0.00 | 0.02 |
| | 0103972 | | | | | | |
| | DSR- | 0.02 | 0.00 | 0.00 | 0.00 | 0.00 | 0.00 |
| | 0103973 | | | | | | |
| 28 days | PBS | 0.00 | 0.00 | 0.00 | 0.00 | 0.00 | 0.00 |
| post | DSR- | 0.11 | 0.02 | 0.06 | 0.03 | 0.04 | 0.05 |
| dosing | 0103301 | | | | | | |
| | DSR- | 0.06 | 0.06 | 0.06 | 0.04 | 0.03 | 0.05 |
| | 0103966 | | | | | | |
| | DSR- | 0.03 | 0.07 | 0.03 | 0.03 | 0.05 | 0.04 |
| | 0103967 | | | | | | |
| | DSR- | 0.00 | 0.01 | 0.00 | 0.00 | 0.02 | 0.01 |
| | 0103968 | | | | | | |
| | DSR- | 0.00 | 0.01 | 0.00 | 0.01 | 0.00 | 0.01 |
| | 0103969 | | | | | | |
| | DSR- | 0.07 | 0.00 | 0.02 | 0.03 | 0.03 | 0.03 |
| | 0103970 | | | | | | |
| | DSR- | 0.05 | 0.03 | 0.01 | 0.03 | 0.02 | 0.03 |
| | 0103971 | | | | | | |
| | DSR- | 0.04 | 0.00 | 0.03 | 0.00 | 0.00 | 0.01 |
| | 0103972 | | | | | | |
| | DSR- | 0.00 | 0.00 | 0.00 | 0.00 | 0.00 | 0.00 |
| | 0103973 | | | | | | |

### Example 15. Provided Oligonucleotides and Compositions are Active In Vivo

All animal procedures were performed under IACUC guidelines. To evaluate the potency and liver exposure of provided oligonucleotides and compositions, male 8-10 weeks of age C57B3L/6 mice were dose at 5 mg/kg on Day 0 by subcutaneous administration. Animals were euthanized on Day 14 or 28 by CO2 asphyxiation followed by thoracotomy and terminal blood collection. After cardiac perfusion with PBS, various tissue samples were harvested and flash-frozen in dry ice. Total RNA was extracted using RNeasy 96 kit (Qiagen), after tissue lysis with TRIzol and chloroform. cDNA production from RNA samples were performed using High-Capacity cDNA Reverse Transcription kit (Thermo Fisher) following manufacturer's instructions and qPCR analysis performed in CFX System using iQ Multiplex Powermix (Bio-Rad for mouse APP mRNA, the following qPCR assay were utilized: mouse Sodl from IDT (Integrated DNA Technologies) qPCR assay ID Mm.PT.58.12368303. Mouse HPRT was used as normalizer (Forward 5′CAAACTTTGCTTTCCCTGGTT3′, Reverse 5′TGGCCTGTATCCAACACTTC3′, Probe 5′/5HEX/ACCAGCAAG/Zen/CTTGCAACCTTAACC/3IABkFQ/3′. Oligonucleotide accumulation was determined by sandwich ELISA.

Ago2 immunoprecipitation assay: Tissues were lysed in lysis buffer 50 mM Tris-HCl at pH 7.5, 200 mM NaCl, 0.5% Triton X-100, 2 mM EDTA, 1 mg/mL heparin) with protease inhibitor (Sigma-Aldrich). Lysate concentration was measured with a protein BCA kit (Pierce BCA protein assay kit or Bradford protein assay kit). Anti-Ago2 antibody was purchased from Wako Chemicals. Control mouse IgG was from eBioscience. Dynabeads (Invitrogen) were used to precipitate antibodies. Ago2-associated siRNA and endogenous miR122 were measured by Stem-Loop RT followed by TaqMan PCR analysis using Taqman miRNA and siRNA assay kit (Thermo fisher) based on manufacturer's methods.

Table 154. shows % mouse SOD1 mRNA remaining relative to PBS control in the quad 14 days post dosing. N=5. N.D.: Not determined.

% remaining of mSOD1 mRNA

| animal No. | PBS | DSR-0103969 | DSR-0104104 |
|---|---|---|---|
| 1 | 102.02 | 70.94 | 45.51 |
| 2 | 101.90 | 65.75 | 59.63 |
| 3 | 106.40 | 72.98 | 55.69 |
| 4 | 99.32 | 63.28 | 59.28 |
| 5 | 90.35 | 53.74 | 47.60 |
| Mean | 100.00 | 65.34 | 53.54 |

Table 155. shows % mouse SOD1 mRNA remaining relative to PBS control in the kidney 14 days post dosing. N=5. N.D.: Not determined.

% remaining of mSOD1 mRNA

| animal No. | PBS | DSR-0103969 | DSR-0104104 |
|---|---|---|---|
| 1 | 90.31 | 63.51 | 41.29 |
| 2 | 105.37 | 49.18 | 33.09 |
| 3 | 89.30 | 72.60 | 35.85 |
| 4 | 124.38 | 51.75 | 35.86 |
| 5 | 90.64 | 69.82 | 33.42 |
| Mean | 100.00 | 61.37 | 35.90 |

Table 156. shows % mouse SOD1 mRNA remaining relative to PBS control in the liver 14 days post dosing. N=5. N.D.: Not determined.

% remaining of mSOD1 mRNA

| animal No. | PBS | DSR-0103969 | DSR-0104104 |
|---|---|---|---|
| 1 | 97.47 | 35.94 | 10.38 |
| 2 | 99.35 | 30.93 | 14.89 |
| 3 | 93.07 | 36.56 | 13.13 |
| 4 | 105.18 | 25.63 | 13.04 |
| 5 | 104.92 | 32.74 | 10.78 |
| Mean | 100.00 | 32.36 | 12.45 |

Table 157. shows % mouse SOD1 mRNA remaining relative to PBS control in the heart 14 days post dosing. N=5. N.D.: Not determined.

% remaining of mSOD1 mRNA

| animal No. | PBS | DSR-0103969 | DSR-0104104 |
|---|---|---|---|
| 1 | 133.88 | 36.44 | 12.29 |
| 2 | 73.22 | 34.16 | 17.01 |
| 3 | 118.66 | 55.04 | 25.54 |
| 4 | 73.57 | 42.10 | 27.69 |
| 5 | 100.67 | 41.99 | 16.11 |
| Mean | 100.00 | 41.95 | 19.73 |

Table 158. shows % mouse SOD1 mRNA remaining relative to PBS control in the lung 14 days post dosing. N=5. N.D.: Not determined.

% remaining of mSOD1 mRNA

| animal No. | PBS | DSR-0103969 | DSR-0104104 |
|---|---|---|---|
| 1 | 111.31 | 93.91 | 77.56 |
| 2 | 103.46 | 79.21 | 53.76 |
| 3 | 105.29 | 96.75 | 58.84 |
| 4 | 92.42 | 92.80 | 82.50 |
| 5 | 87.53 | 100.69 | 70.26 |
| Mean | 100.00 | 92.67 | 68.59 |

Table 159. shows % mouse SOD1 mRNA remaining relative to PBS control in the brown adipose tissue 14 days post dosing. N=5. N.D.: Not determined.

% remaining of mSOD1 mRNA

| animal No. | PBS | DSR-0103969 | DSR-0104104 |
|---|---|---|---|
| 1 | 93.39 | 39.84 | 31.66 |
| 2 | 105.89 | 53.20 | 22.30 |
| 3 | 93.48 | 38.48 | 30.02 |
| 4 | 117.69 | 42.93 | 13.55 |
| 5 | 89.55 | 38.53 | 10.96 |
| Mean | 100.00 | 42.60 | 21.70 |

Table 160. shows % mouse SOD1 mRNA remaining relative to PBS control in the white adipose tissue 14 days post dosing. N=5. N.D.: Not determined.

% remaining of mSOD1 mRNA

| animal No. | PBS | DSR-0103969 | DSR-0104104 |
|---|---|---|---|
| 1 | 120.40 | 30.58 | 13.10 |
| 2 | 93.83 | 28.11 | 63.67 |
| 3 | 106.73 | 43.07 | 16.18 |
| 4 | 95.02 | 30.94 | 13.59 |
| 5 | 84.02 | 40.94 | 16.89 |
| Mean | 100.00 | 34.73 | 24.69 |

Table 161. shows % mouse SOD1 mRNA remaining relative to PBS control in the sciatic nerve 14 days post dosing. N=5. N.D. Not determined.

% remaining of mSOD1 mRNA

| animal No. | PBS | DSR-0103969 | DSR-0104104 |
|---|---|---|---|
| 1 | 94.08 | 155.52 | 104.44 |
| 2 | 97.98 | 126.78 | 98.92 |
| 3 | 90.62 | 95.06 | 120.87 |
| 4 | 108.16 | 120.99 | 95.01 |
| 5 | 109.15 | 116.63 | 85.83 |
| Mean | 100.00 | 123.00 | 101.01 |

Table 162. shows % mouse SOD1 mRNA remaining relative to PBS control in the diaphragm 14 days post dosing. N=5. N.D.; Not determined.

% remaining of mSOD1 mRNA

| animal No. | PBS | DSR-0103969 | DSR-0104104 |
|---|---|---|---|
| 1 | 117.76 | 48.63 | 32.82 |
| 2 | 115.31 | 54.70 | 34.53 |
| 3 | 105.64 | 51.48 | 29.51 |
| 4 | 74.27 | 41.64 | 20.87 |
| 5 | 87.02 | 36.54 | 26.86 |
| Mean | 100.00 | 46.60 | 28.92 |

Table 163. shows % mouse SOD1 mRNA remaining relative to PBS control in the quad 28 days post dosing. N=5. N.D.: Not determined.

% remaining of mSOD1 mRNA

| animal No. | PBS | DSR-0103969 | DSR-0104104 |
|---|---|---|---|
| 1 | 106.41 | 55.01 | 43.53 |
| 2 | 101.01 | 55.39 | 52.78 |
| 3 | 97.16 | 55.83 | 46.02 |
| 4 | 96.83 | 56.96 | 56.25 |
| 5 | 98.59 | 57.81 | 53.04 |
| Mean | 100.00 | 56.20 | 50.32 |

Table 164. shows % mouse SOD1 mRNA remaining relative to PBS control in the kidney 28 days post dosing. N=5. N.D.: Not determined.

% remaining of mSOD1 mRNA

| animal No. | PBS | DSR-0103969 | DSR-0104104 |
|---|---|---|---|
| 1 | 116.21 | 63.06 | 86.05 |
| 2 | 100.07 | 71.17 | 66.88 |
| 3 | 113.33 | 92.58 | 74.60 |
| 4 | 125.10 | 108.89 | 66.64 |
| 5 | 45.28 | 80.55 | 83.29 |
| Mean | 100.00 | 83.25 | 75.49 |

Table 165. shows % mouse SOD1 mRNA remaining relative to PBS control in the liver 28 days post dosing. N=5. N.D.: Not determined.

% remaining of mSOD1 mRNA

| animal No. | PBS | DSR-0103969 | DSR-0104104 |
|---|---|---|---|
| 1 | 100.18 | 26.85 | 20.53 |
| 2 | 100.78 | 28.22 | 13.20 |
| 3 | 89.83 | 36.84 | 12.02 |
| 4 | 107.46 | 23.22 | 12.82 |
| 5 | 101.74 | 27.66 | 20.87 |
| Mean | 100.00 | 28.56 | 15.89 |

Table 166. shows % mouse SOD1 mRNA remaining relative to PBS control in the heart 28 days post dosing. N=5. N.D.: Not determined.

% remaining of mSOD1 mRNA

| animal No. | PBS | DSR-0103969 | DSR-0104104 |
|---|---|---|---|
| 1 | 84.12 | 31.31 | 15.89 |
| 2 | 111.47 | 44.03 | 16.94 |
| 3 | 119.16 | 46.17 | 18.04 |
| 4 | 99.35 | 37.37 | 13.60 |
| 5 | 85.89 | 44.04 | 23.76 |
| Mean | 100.00 | 40.58 | 17.65 |

Table 167. shows % mouse SOD1 mRNA remaining relative to PBS control in the lung 28 days post dosing. N=5. N.D.: Not determined.

% remaining of mSOD1 mRNA

| animal No. | PBS | DSR-0103969 | DSR-0104104 |
|---|---|---|---|
| 1 | 66.59 | 112.11 | 51.64 |
| 2 | 94.07 | 87.96 | 77.26 |
| 3 | 111.54 | 114.55 | 61.94 |
| 4 | 150.46 | 120.53 | 71.26 |
| 5 | 77.34 | 97.47 | 71.83 |
| Mean | 100.00 | 106.52 | 66.79 |

Table 168. shows % mouse SOD1 mRNA remaining relative to PBS control in the brown adipose tissue 28 days post dosing. N=5. N.D.: Not determined.

% remaining of mSOD1 mRNA

| animal No. | PBS | DSR-0103969 | DSR-0104104 |
|---|---|---|---|
| 1 | 119.54 | 38.62 | 27.28 |
| 2 | 114.41 | 34.32 | 29.17 |
| 3 | 63.98 | 58.81 | 17.27 |
| 4 | 124.76 | 33.65 | 20.21 |
| 5 | 77.32 | 47.92 | 18.77 |
| Mean | 100.00 | 42.66 | 22.54 |

Table 169. shows % mouse SOD1 mRNA remaining relative to PBS control in the white adipose tissue 28 days post dosing. N=5. N.D.: Not determined.

% remaining of mSOD1 mRNA

| animal No. | PBS | DSR-0103969 | DSR-0104104 |
|---|---|---|---|
| 1 | 86.47 | 37.33 | 28.58 |
| 2 | 113.09 | 55.53 | 28.38 |
| 3 | 88.82 | 34.94 | 97.75 |
| 4 | 97.09 | 34.02 | 48.66 |
| 5 | 114.53 | 42.00 | 29.26 |
| Mean | 100.00 | 40.76 | 46.52 |

Table 170. shows % mouse SOD1 mRNA remaining relative to PBS control in the sciatic nerve 28 days post dosing. N=5. N.D.: Not determined.

% remaining of mSOD1 mRNA

| animal No. | PBS | DSR-0103969 | DSR-0104104 |
|---|---|---|---|
| 1 | 126.48 | 103.19 | 104.99 |
| 2 | 81.53 | 131.79 | 64.07 |
| 3 | 72.46 | 100.85 | 112.64 |
| 4 | 114.48 | 123.10 | 63.15 |
| 5 | 105.05 | 80.28 | 98.09 |
| Mean | 100.00 | 107.84 | 88.59 |

Table 171. shows % mouse SOD1 mRNA remaining relative to PBS control in the diaphragm 28 days post dosing. N=5. N.D.: Not determined.

% remaining of mSOD1 mRNA

| animal No. | PBS | DSR-0103969 | DSR-0104104 |
|---|---|---|---|
| 1 | 122.75 | 43.12 | 30.26 |
| 2 | 70.63 | 36.51 | 26.31 |
| 3 | 105.65 | 37.63 | 22.41 |
| 4 | 103.22 | 38.43 | 23.78 |
| 5 | 97.74 | 44.76 | 30.83 |
| Mean | 100.00 | 40.09 | 26.72 |

Table 171a. shows Ago2 loading of guide strand in the liver relative to miR-143 in 5 mg/kg treated mouse cohort 14 days and 28 days post dose

| | Ct: | Ct: | Ct: | Ct: | Relative |
|---|---|---|---|---|---|
| | mSOD1/ | miR- | mSOD1/ | miR- | mSOD1/miR- |
| | Ago2 | 143/Ago2 | IgG | 143/IgG | 143 |
| | Day 14 | | | | |
| PBS-1 | 35.13 | 28.37 | 35.27 | 37.61 | 0.002 |
| PBS-2 | 34.78 | 27.56 | 34.54 | 35.97 | 0.002 |
| PBS-3 | 35.24 | 28.27 | 34.32 | 37.63 | 0.001 |
| DSR- | 31.46 | 28.63 | 33.54 | 37.73 | 0.008 |
| 0103969- | | | | | |
| 1 | | | | | |
| DSR- | 31.65 | 29.02 | 33.8 | 36.22 | 0.030 |
| 0103969- | | | | | |
| 2 | | | | | |
| DSR- | 31.61 | 28.31 | 34.99 | 39.55 | 0.004 |
| 0103969- | | | | | |
| 3 | | | | | |
| DSR- | 28.86 | 28.42 | 32.79 | 37.09 | 0.037 |
| 0104104- | | | | | |
| 1 | | | | | |
| DSR- | 29.84 | 28.86 | 33.31 | 37.01 | 0.039 |
| 0104104- | | | | | |
| 2 | | | | | |
| DSR- | 29.48 | 28.31 | 33.09 | 37.65 | 0.019 |
| 0104104- | | | | | |
| 3 | | | | | |
| | Day 28 | | | | |
| DSR- | 32.43 | 28.48 | 34.77 | 38.25 | 0.006 |
| 0103969- | | | | | |
| 1 | | | | | |
| DSR- | 32.84 | 28.6 | 34.19 | 38.04 | 0.004 |
| 0103969- | | | | | |
| 2 | | | | | |
| DSR- | 33.95 | 30.09 | 34.48 | 37.43 | 0.009 |
| 0103969- | | | | | |
| 3 | | | | | |
| DSR- | 28.04 | 28.03 | 31.56 | 37.87 | 0.013 |
| 0104104- | | | | | |
| 1 | | | | | |
| DSR- | 27.88 | 27.68 | 33.58 | 35.89 | 0.176 |
| 0104104- | | | | | |
| 2 | | | | | |
| DSR- | 28.8 | 28.31 | 32.87 | 36.95 | 0.042 |
| 0104104- | | | | | |
| 3 | | | | | |

Table 171b. shows Ago2 loading of guide strand in the kidney relative to miR-143 in 5 mg/kg treated mouse cohort 14 days and 28 days post dose

| | Ct: | Ct: | Ct: | Ct: | Relative |
|---|---|---|---|---|---|
| | mSOD1/ | miR- | mSOD1/ | miR- | mSOD1/miR- |
| | Ago2 | 143/Ago2 | IgG | 143/IgG | 143 |
| | Day 14 | | | | |
| PBS-1 | 34.01 | 30.88 | 34.04 | 34.79 | 0.068 |
| PBS-2 | 34.37 | 30.78 | 34.5 | 34.64 | 0.075 |
| PBS-3 | 34.06 | 29.91 | 34.66 | 34.68 | 0.056 |
| DSR- | 31.07 | 30.28 | 34.29 | 35.69 | 0.219 |
| 0103969- | | | | | |
| 1 | | | | | |
| DSR- | 32.46 | 30.89 | 33.65 | 35.18 | 0.117 |
| 0103969- | | | | | |
| 2 | | | | | |
| DSR- | 32.26 | 30.43 | 32.64 | 35.18 | 0.048 |
| 0103969- | | | | | |
| 3 | | | | | |
| DSR- | 31.18 | 30.34 | 32.72 | 35.6 | 0.076 |
| 0104104- | | | | | |
| 1 | | | | | |
| DSR- | 30.75 | 30.07 | 32.82 | 34.31 | 0.222 |
| 0104104- | | | | | |
| 2 | | | | | |
| DSR- | 31.72 | 30.78 | 34.14 | 35.8 | 0.165 |
| 0104104- | | | | | |
| 3 | | | | | |
| | Day 28 | | | | |
| DSR- | 31.66 | 31.26 | 31.33 | 34.65 | 0.076 |
| 0103969- | | | | | |
| 1 | | | | | |
| DSR- | 32.67 | 31.97 | 32.48 | 34.48 | 0.154 |
| 0103969- | | | | | |
| 2 | | | | | |
| DSR- | 31.99 | 30.91 | 32.68 | 35.17 | 0.084 |
| 0103969- | | | | | |
| 3 | | | | | |
| DSR- | 30.47 | 30.68 | 31.89 | 35.43 | 0.099 |
| 0104104- | | | | | |
| 1 | | | | | |
| DSR- | 31.33 | 31.59 | 32.11 | 36.16 | 0.072 |
| 0104104- | | | | | |
| 2 | | | | | |
| DSR- | 30.93 | 30.66 | 31.09 | 34.45 | 0.081 |
| 0104104- | | | | | |
| 3 | | | | | |

Table 171c. shows Ago2 loading of guide strand in the white adipose tissue relative to miR-143 in 5 mg/kg treated mouse cohort 14 days and 28 days post dose

| | Ct: | Ct: | Ct: | Ct: | Relative |
|---|---|---|---|---|---|
| | mSOD1/ | miR- | mSOD1/ | miR- | mSOD1/miR- |
| | Ago2 | 143/Ago2 | IgG | 143/IgG | 143 |
| | Day 14 | | | | |
| PBS-1 | 33.88 | 28.25 | 35.85 | 36.21 | 0.016 |
| PBS-2 | 35.72 | 30.36 | 35.45 | 34.88 | 0.036 |
| PBS-3 | 34.60 | 29.23 | 35.34 | 35.00 | 0.031 |
| DSR- | 31.18 | 29.28 | 36.02 | 35.88 | 0.295 |
| 0103969- | | | | | |
| 1 | | | | | |
| DSR- | 31.47 | 30.05 | 32.86 | 36.15 | 0.038 |
| 0103969- | | | | | |
| 2 | | | | | |
| DSR- | 30.85 | 29.50 | 33.55 | 34.80 | 0.165 |
| 0103969- | | | | | |
| 3 | | | | | |
| DSR- | 29.58 | 29.68 | 33.44 | 35.47 | 0.262 |
| 0104104- | | | | | |
| 1 | | | | | |
| DSR- | 28.89 | 29.16 | 33.69 | 36.26 | 0.203 |
| 0104104- | | | | | |
| 2 | | | | | |
| DSR- | 28.23 | 29.50 | 32.77 | 34.83 | 0.578 |
| 0104104- | | | | | |
| 3 | | | | | |
| | Day 28 | | | | |
| DSR- | 31.26 | 28.84 | 34.44 | 34.88 | 0.138 |
| 0103969- | | | | | |
| 1 | | | | | |
| DSR- | 32.81 | 29.77 | 34.81 | 34.82 | 0.121 |
| 0103969- | | | | | |
| 2 | | | | | |
| DSR- | 36.11 | 31.05 | 34.29 | 34.74 | 0.022 |
| 0103969- | | | | | |
| 3 | | | | | |
| DSR- | 28.28 | 29.29 | 32.93 | 34.61 | 0.629 |
| 0104104- | | | | | |
| 1 | | | | | |
| DSR- | 27.20 | 27.24 | 32.88 | 33.55 | 0.646 |
| 0104104- | | | | | |
| 2 | | | | | |
| DSR- | 28.03 | 28.09 | 33.09 | 33.94 | 0.578 |
| 0104104- | | | | | |
| 3 | | | | | |

### Example 16. Provided Oligonucleotides and Compositions are Active In Vitro

Various siRNAs for mouse TTR were designed and constructed. A number of siRNAs were tested in vitro in mouse primary hepatocytes at one or a range of concentrations.

Example protocol for in vitro determination of siRNA activity in mouse primary hepatocytes: For determination of siRNAs activity, siRNAs at specific concentration were gymnotically delivered to mouse primary hepatocytes plated at 96-well plates, with 10,000 cells/well. Following 48 hours treatment, total RNA was extracted using SV96 Total RNA Isolation kit (Promega). cDNA production from RNA samples were performed using High-Capacity cDNA Reverse Transcription kit (Thermo Fisher) following manufacturer's instructions and qPCR analysis performed in CFX System using iQ Multiplex Powermix (Bio-Rad). For mouse TTR mRNA, the following qPCR assay were utilized: IDT PrimeTime predesigned qPCR Assay Mm.PT.58.11922308. Mouse HPRT was used as normalizer (Mm.PT.39a.22214828). mRNA knockdown levels were calculated as % mRNA remaining relative to mock treatment.

Table 172. shows 0% mouse TTR mRNA remaining (at 500 pM, 150 pM and 50 pM siRNA treatment) relative to mouse HPRT control. N=2. N.D.: Not determined.

| | 500 pM | | | 150 pM | | |
|---|---|---|---|---|---|---|
| | % remaining | % remaining | | % remaining | % remaining | |
| | mRNA | mRNA | | mRNA | mRNA | |
| | (mTTR/ | (mTTR/ | | (mTTR/ | (mTTR/ | |
| siRNA | mHPRT) − 1 | mHPRT) − 2 | Mean | mHPRT) − 1 | mHPRT) − 2 | Mean |
| DSR- | 44.914 | 13.442 | 29.18 | 61.248 | 37.444 | 49.35 |
| 0103330 | | | | | | |
| DSR- | 40.223 | 18.057 | 29.14 | 63.607 | 38.704 | 51.16 |
| 0103329 | | | | | | |
| DSR- | 53.107 | 18.136 | 35.62 | 62.262 | 38.074 | 50.17 |
| 0103328 | | | | | | |
| DSR- | 50.343 | 19.86 | 35.10 | 56.943 | 43.087 | 50.02 |
| 0103327 | | | | | | |
| DSR- | 24.61 | 6.02 | 15.32 | 34.349 | 25.768 | 30.06 |
| 0102524 | | | | | | |

| | 150 pM | | |
|---|---|---|---|
| | % remaining | % remaining | |
| | mRNA | mRNA | |
| | (mTTR/ | (mTTR/ | |
| siRNA | mHPRT) − 1 | mHPRT) − 2 | Mean |
| DSR- | 74.715 | 39.185 | 56.95 |
| 0103330 | | | |
| DSR- | 88.027 | 51.044 | 69.54 |
| 0103329 | | | |
| DSR- | 72.845 | 58.895 | 65.87 |
| 0103328 | | | |
| DSR- | 76.226 | 48.962 | 62.59 |
| 0103327 | | | |
| DSR- | 49.451 | 32.499 | 40.98 |
| 0102524 | | | |

Table 173. shows % mouse TTR mRNA remaining (at 500 pM, 150 pM and 50 pM siRNA treatment) relative to mouse HPRT control. N=2. N.D.: Not determined.

| | 500 pM | | | 150 pM | | |
|---|---|---|---|---|---|---|
| | % remaining | % remaining | | % remaining | % remaining | |
| | mRNA | mRNA | | mRNA | mRNA | |
| | (mTTR/ | (mTTR/ | | (mTTR/ | (mTTR/ | |
| siRNA | mHPRT) − 1 | mHPRT) − 2 | Mean | mHPRT) − 1 | mHPRT) − 2 | Mean |
| DSR- | 63.66 | 31.39 | 47.52 | 84.45 | 82.49 | 83.47 |
| 0103370- | | | | | | |
| 001-001 | | | | | | |
| DSR- | 37.34 | 13.41 | 25.37 | 76.07 | 56.59 | 66.33 |
| 0103028- | | | | | | |
| 010-001 | | | | | | |
| DSR- | 47.96 | 38.22 | 43.09 | 83.56 | 65.42 | 74.49 |
| 0103393- | | | | | | |
| 001-001 | | | | | | |
| DSR- | 29.32 | 17.64 | 23.48 | 65.62 | 43.76 | 54.69 |
| 0103392- | | | | | | |
| 001-001 | | | | | | |
| DSR- | 35.52 | 13.59 | 24.55 | 82.44 | 53.13 | 67.79 |
| 0103391- | | | | | | |
| 001-001 | | | | | | |
| DSR- | 37.87 | 14.15 | 26.01 | 82.49 | 57.92 | 70.21 |
| 0103368- | | | | | | |
| 001-001 | | | | | | |
| DSR- | 47.48 | 26.76 | 37.12 | 83.56 | 51.44 | 67.50 |
| 0103390- | | | | | | |
| 001-001 | | | | | | |
| DSR- | 40.44 | 25.79 | 33.12 | 72.90 | 58.79 | 65.85 |
| 0103380- | | | | | | |
| 001-001 | | | | | | |
| DSR- | 36.17 | 12.88 | 24.53 | 63.33 | 63.40 | 63.36 |
| 0103367- | | | | | | |
| 001-001 | | | | | | |
| DSR- | 43.64 | 23.82 | 33.73 | 72.78 | 69.23 | 71.01 |
| 0103372- | | | | | | |
| 001-001 | | | | | | |
| DSR- | 72.04 | 55.84 | 63.94 | 100.89 | 90.65 | 95.77 |
| 0103397- | | | | | | |
| 001-001 | | | | | | |
| DSR- | 61.28 | 29.19 | 45.23 | 79.97 | 76.43 | 78.20 |
| 0103363- | | | | | | |
| 001-001 | | | | | | |
| DSR- | 56.24 | 22.45 | 39.34 | 90.12 | 74.89 | 82.50 |
| 0103396- | | | | | | |
| 001-001 | | | | | | |
| DSR- | 45.97 | 20.13 | 33.05 | 90.30 | 64.04 | 77.17 |
| 0103366- | | | | | | |
| 001-001 | | | | | | |
| DSR- | 72.99 | 42.47 | 57.73 | 106.82 | 75.99 | 91.40 |
| 0103365- | | | | | | |
| 001-001 | | | | | | |
| DSR- | 39.85 | 30.43 | 35.14 | 81.03 | 64.18 | 72.60 |
| 0103379- | | | | | | |
| 001-001 | | | | | | |
| DSR- | 56.05 | 12.40 | 34.23 | 75.53 | 91.26 | 83.39 |
| 0103389- | | | | | | |
| 001-001 | | | | | | |
| DSR- | 35.87 | 19.91 | 27.89 | 77.15 | 68.67 | 72.91 |
| 0103378- | | | | | | |
| 001-001 | | | | | | |
| DSR- | 62.41 | 51.39 | 56.90 | 101.18 | 85.47 | 93.32 |
| 0103377- | | | | | | |
| 001-001 | | | | | | |
| DSR- | 104.43 | 110.61 | 107.52 | 94.10 | 95.24 | 94.67 |
| 0103376- | | | | | | |
| 001-001 | | | | | | |
| DSR- | 33.45 | 14.85 | 24.15 | 80.81 | 67.21 | 74.01 |
| 0103395- | | | | | | |
| 001-001 | | | | | | |
| DSR- | 15.78 | 12.43 | 14.11 | 59.69 | 41.97 | 50.83 |
| 0102524- | | | | | | |
| 014-001 | | | | | | |

| | 50 pM | | |
|---|---|---|---|
| | % remaining | % remaining | |
| | mRNA | mRNA | |
| | (mTTR/ | (mTTR/ | |
| siRNA | mHPRT) − 1 | mHPRT) − 2 | Mean |
| DSR- | 75.91 | 88.05 | 81.98 |
| 0103370- | | | |
| 001-001 | | | |
| DSR- | 59.64 | 80.80 | 70.22 |
| 0103028- | | | |
| 010-001 | | | |
| DSR- | 52.18 | 50.23 | 51.20 |
| 0103393- | | | |
| 001-001 | | | |
| DSR- | 56.03 | 54.13 | 55.08 |
| 0103392- | | | |
| 001-001 | | | |
| DSR- | 67.42 | 67.11 | 67.27 |
| 0103391- | | | |
| 001-001 | | | |
| DSR- | 62.35 | 67.23 | 64.79 |
| 0103368- | | | |
| 001-001 | | | |
| DSR- | 81.66 | 84.30 | 82.98 |
| 0103390- | | | |
| 001-001 | | | |
| DSR- | 91.97 | 92.40 | 92.19 |
| 0103380- | | | |
| 001-001 | | | |
| DSR- | 63.14 | 57.91 | 60.53 |
| 0103367- | | | |
| 001-001 | | | |
| DSR- | 70.03 | 69.09 | 69.56 |
| 0103372- | | | |
| 001-001 | | | |
| DSR- | 76.44 | 77.11 | 76.78 |
| 0103397- | | | |
| 001-001 | | | |
| DSR- | 83.23 | 72.99 | 78.11 |
| 0103363- | | | |
| 001-001 | | | |
| DSR- | 73.89 | 90.45 | 82.17 |
| 0103396- | | | |
| 001-001 | | | |
| DSR- | 69.56 | 83.56 | 76.56 |
| 0103366- | | | |
| 001-001 | | | |
| DSR- | 93.25 | 107.55 | 100.40 |
| 0103365- | | | |
| 001-001 | | | |
| DSR- | 80.16 | 102.49 | 91.33 |
| 0103379- | | | |
| 001-001 | | | |
| DSR- | 73.71 | 53.61 | 63.66 |
| 0103389- | | | |
| 001-001 | | | |
| DSR- | 62.78 | 56.42 | 59.60 |
| 0103378- | | | |
| 001-001 | | | |
| DSR- | 72.39 | 66.57 | 69.48 |
| 0103377- | | | |
| 001-001 | | | |
| DSR- | 88.23 | 90.90 | 89.57 |
| 0103376- | | | |
| 001-001 | | | |
| DSR- | 66.75 | 76.92 | 71.84 |
| 0103395- | | | |
| 001-001 | | | |
| DSR- | 50.86 | 82.76 | 66.81 |
| 0102524- | | | |
| 014-001 | | | |

Table 174. shows % mouse TTR mRNA remaining (at 500 pM, 150 pM and 50 pM siRNA treatment) relative to mouse HPRT control. N=2. N.D.: Not determined.

| | 500 pM | | | 150 pM | | |
|---|---|---|---|---|---|---|
| | % remaining | % remaining | | % remaining | % remaining | |
| | mRNA | mRNA | | mRNA | mRNA | |
| | (mTTR/ | (mTTR/ | | (mTTR/ | (mTTR/ | |
| siRNA | mHPRT) − 1 | mHPRT) − 2 | Mean | mHPRT) − 1 | mHPRT) − 2 | Mean |
| DSR- | 46.19 | 32.66 | 39.42 | 76.73 | 69.64 | 73.18 |
| 0103385- | | | | | | |
| 001-001 | | | | | | |
| DSR- | 56.78 | 47.17 | 51.97 | 83.86 | 81.04 | 82.45 |
| 0103375- | | | | | | |
| 001-001 | | | | | | |
| DSR- | 29.08 | 24.69 | 26.89 | 61.39 | 52.44 | 56.91 |
| 0103374- | | | | | | |
| 001-001 | | | | | | |
| DSR- | 34.47 | 28.43 | 31.45 | 69.52 | 60.36 | 64.94 |
| 0103384- | | | | | | |
| 001-001 | | | | | | |
| DSR- | 31.11 | 17.55 | 24.33 | 60.40 | 45.28 | 52.84 |
| 0103388- | | | | | | |
| 001-001 | | | | | | |
| DSR- | 41.88 | 21.61 | 31.74 | 91.27 | 63.17 | 77.22 |
| 0103373- | | | | | | |
| 001-001 | | | | | | |
| DSR- | 39.69 | 18.78 | 29.23 | 73.72 | 51.10 | 62.41 |
| 0103383- | | | | | | |
| 001-001 | | | | | | |
| DSR- | 50.75 | 34.12 | 42.43 | 77.93 | 67.21 | 72.57 |
| 0103364- | | | | | | |
| 001-001 | | | | | | |
| DSR- | 32.34 | 27.16 | 29.75 | 67.23 | 55.40 | 61.31 |
| 0103394- | | | | | | |
| 001-001 | | | | | | |
| DSR- | 35.48 | 20.81 | 28.15 | 67.58 | 64.45 | 66.02 |
| 0103382- | | | | | | |
| 001-001 | | | | | | |
| DSR- | 31.57 | 21.62 | 26.60 | 63.28 | 50.26 | 56.77 |
| 0103371- | | | | | | |
| 001-001 | | | | | | |
| DSR- | 15.78 | 12.43 | 14.11 | 59.69 | 41.97 | 50.83 |
| 0102524- | | | | | | |
| 014-001 | | | | | | |

| | 50 pM | | |
|---|---|---|---|
| | % remaining | % remaining | |
| | mRNA | mRNA | |
| | (mTTR/ | (mTTR/ | |
| siRNA | mHPRT) − 1 | mHPRT) − 2 | Mean |
| DSR- | 89.76 | 86.30 | 88.03 |
| 0103385- | | | |
| 001-001 | | | |
| DSR- | 74.30 | 77.99 | 76.14 |
| 0103375- | | | |
| 001-001 | | | |
| DSR- | 57.03 | 64.71 | 60.87 |
| 0103374- | | | |
| 001-001 | | | |
| DSR- | 55.88 | 60.95 | 58.42 |
| 0103384- | | | |
| 001-001 | | | |
| DSR- | 60.77 | 56.25 | 58.51 |
| 0103388- | | | |
| 001-001 | | | |
| DSR- | 79.91 | 82.16 | 81.04 |
| 0103373- | | | |
| 001-001 | | | |
| DSR- | 79.95 | 74.08 | 77.01 |
| 0103383- | | | |
| 001-001 | | | |
| DSR- | 93.41 | 85.81 | 89.61 |
| 0103364- | | | |
| 001-001 | | | |
| DSR- | 88.59 | 89.65 | 89.12 |
| 0103394- | | | |
| 001-001 | | | |
| DSR- | 76.96 | 67.49 | 72.23 |
| 0103382- | | | |
| 001-001 | | | |
| DSR- | 59.24 | 66.84 | 63.04 |
| 0103371- | | | |
| 001-001 | | | |
| DSR- | 50.86 | 82.76 | 66.81 |
| 0102524- | | | |
| 014-001 | | | |

Table 175. shows % mouse TTR mRNA remaining (at 500 pM, 150 pM and 50 pM siRNA treatment) relative to mouse HPRT control. N=2. N.D.: Not determined.

| | 500 pM | | | 150 pM | | |
|---|---|---|---|---|---|---|
| | % remaining | % remaining | | % remaining | % remaining | |
| | mRNA | mRNA | | mRNA | mRNA | |
| | (mTTR/ | (mTTR/ | | (mTTR/ | (mTTR/ | |
| siRNA | mHPRT) − 1 | mHPRT) − 2 | Mean | mHPRT) − 1 | mHPRT) − 2 | Mean |
| DSR- | 59.82 | 39.42 | 49.62 | 93.05 | 77.15 | 85.10 |
| 0103387- | | | | | | |
| 001-001 | | | | | | |
| DSR- | 61.51 | 42.14 | 51.83 | 87.76 | 71.35 | 79.55 |
| 0103381- | | | | | | |
| 001-001 | | | | | | |
| DSR- | 77.05 | 57.43 | 67.24 | 96.11 | 80.94 | 88.53 |
| 0103386- | | | | | | |
| 001-001 | | | | | | |
| DSR- | 65.22 | 57.73 | 61.47 | 93.47 | 76.52 | 84.99 |
| 0103369- | | | | | | |
| 001-001 | | | | | | |
| DSR- | 23.84 | 14.11 | 18.98 | 51.07 | 33.24 | 42.16 |
| 0103099- | | | | | | |
| 010-001 | | | | | | |
| DSR- | 15.78 | 12.43 | 14.11 | 59.69 | 41.97 | 50.83 |
| 0102524- | | | | | | |
| 014-001 | | | | | | |

| | 50 pM | | |
|---|---|---|---|
| | % remaining | % remaining | |
| | mRNA | mRNA | |
| | (mTTR/ | (mTTR/ | |
| siRNA | mHPRT) − 1 | mHPRT) − 2 | Mean |
| DSR- | 79.56 | 90.29 | 84.92 |
| 0103387- | | | |
| 001-001 | | | |
| DSR- | 67.33 | 64.11 | 65.72 |
| 0103381- | | | |
| 001-001 | | | |
| DSR- | 116.43 | 94.04 | 105.24 |
| 0103386- | | | |
| 001-001 | | | |
| DSR- | 91.10 | 85.91 | 88.51 |
| 0103369- | | | |
| 001-001 | | | |
| DSR- | 69.86 | 59.10 | 64.48 |
| 0103099- | | | |
| 010-001 | | | |
| DSR- | 50.86 | 82.76 | 66.81 |
| 0102524- | | | |
| 014-001 | | | |

Table 176. shows % mouse TTR mRNA remaining (at 500 pM, 150 pM and 50 pM siRNA treatment) relative to mouse HPRT control. N=2. N.D.: Not determined.

| | 500 pM | | | 150 pM | | |
|---|---|---|---|---|---|---|
| | % remaining | % remaining | | % remaining | % remaining | |
| | mRNA | mRNA | | mRNA | mRNA | |
| | (mTTR/ | (mTTR/ | | (mTTR/ | (mTTR/ | |
| siRNA | mHPRT) − 1 | mHPRT) − 2 | Mean | mHPRT) − 1 | mHPRT) − 2 | Mean |
| DSR- | 21.85 | 20.66 | 21.25 | 54.92 | 57.17 | 56.04 |
| 0104178 | | | | | | |
| DSR- | 39.29 | 32.72 | 36.00 | 71.95 | 72.27 | 72.11 |
| 0104177 | | | | | | |
| DSR- | 11.07 | 8.49 | 9.78 | 36.56 | 40.84 | 38.70 |
| 0104176 | | | | | | |
| DSR- | 18.55 | 14.18 | 16.37 | 47.85 | 39.74 | 43.80 |
| 0104175 | | | | | | |
| DSR- | 18.42 | 13.52 | 15.97 | 49.68 | 43.54 | 46.61 |
| 0104174 | | | | | | |
| DSR- | 78.56 | 70.27 | 74.42 | 106.46 | 89.22 | 97.84 |
| 0104173 | | | | | | |
| DSR- | 31.05 | 27.79 | 29.42 | 69.97 | 55.37 | 62.67 |
| 0104172 | | | | | | |
| DSR- | 11.72 | 5.58 | 8.65 | 28.88 | 26.76 | 27.82 |
| 0104171 | | | | | | |
| DSR- | 9.56 | 4.44 | 7.00 | 29.73 | 21.58 | 25.65 |
| 0102626 | | | | | | |
| DSR- | 6.26 | 4.21 | 5.23 | 19.95 | 22.30 | 21.13 |
| 0102524 | | | | | | |

| | 50 pM | | |
|---|---|---|---|
| | % remaining | % remaining | |
| | mRNA | mRNA | |
| | (mTTR/ | (mTTR/ | |
| siRNA | mHPRT) − 1 | mHPRT) − 2 | Mean |
| DSR- | 66.46 | 63.38 | 64.92 |
| 0104178 | | | |
| DSR- | 79.42 | 87.51 | 83.46 |
| 0104177 | | | |
| DSR- | 56.94 | 43.39 | 50.16 |
| 0104176 | | | |
| DSR- | 61.55 | 42.48 | 52.01 |
| 0104175 | | | |
| DSR- | 67.60 | 52.03 | 59.81 |
| 0104174 | | | |
| DSR- | 94.05 | 86.83 | 90.44 |
| 0104173 | | | |
| DSR- | 64.26 | 70.29 | 67.28 |
| 0104172 | | | |
| DSR- | 38.01 | 34.84 | 36.43 |
| 0104171 | | | |
| DSR- | 43.41 | 40.04 | 41.72 |
| 0102626 | | | |
| DSR- | 50.50 | 36.79 | 43.65 |
| 0102524 | | | |

Table 177. shows % mouse TTR mRNA remaining (at 500 pM, 150 pM and 50 pM siRNA treatment) relative to mouse HPRT control. N=2. N.D.: Not determined.

| | 500 pM | | | 150 pM | | |
|---|---|---|---|---|---|---|
| | % remaining | % remaining | | % remaining | % remaining | |
| | mRNA | mRNA | | mRNA | mRNA | |
| | (mTTR/ | (mTTR/ | | (mTTR/ | (mTTR/ | |
| siRNA | mHPRT) − 1 | mHPRT) − 2 | Mean | mHPRT) − 1 | mHPRT) − 2 | Mean |
| DSR- | 8.01 | 5.16 | 6.59 | 21.99 | 20.84 | 21.41 |
| 0104187 | | | | | | |
| DSR- | 61.87 | 46.53 | 54.20 | 80.93 | 81.97 | 81.45 |
| 0104184 | | | | | | |
| DSR- | 25.11 | 16.72 | 20.92 | 55.65 | 54.04 | 54.84 |
| 0104183 | | | | | | |
| DSR- | 20.82 | 14.98 | 17.90 | 53.77 | 49.97 | 51.87 |
| 0104181 | | | | | | |
| DSR- | 9.56 | 4.44 | 7.00 | 29.73 | 21.58 | 25.65 |
| 0102626 | | | | | | |
| DSR- | 6.26 | 4.21 | 5.23 | 19.95 | 22.30 | 21.13 |
| 0102524 | | | | | | |

| | 50 pM | | |
|---|---|---|---|
| | % remaining | % remaining | |
| | mRNA | mRNA | |
| | (mTTR/ | (mTTR/ | |
| siRNA | mHPRT) − 1 | mHPRT) − 2 | Mean |
| DSR- | 47.64 | 51.08 | 49.36 |
| 0104187 | | | |
| DSR- | 96.30 | 86.65 | 91.48 |
| 0104184 | | | |
| DSR- | 68.28 | 60.71 | 64.50 |
| 0104183 | | | |
| DSR- | 63.27 | 59.44 | 61.35 |
| 0104181 | | | |
| DSR- | 43.41 | 40.04 | 41.72 |
| 0102626 | | | |
| DSR- | 50.50 | 36.79 | 43.65 |
| 0102524 | | | |

Table 178 shows % mouse TTR mRNA remaining (at 500 pM, 150 pM and 50 pM siRNA treatment) relative to mouse HPRT control. N=2. N.D.: Not determined.

| | 500 pM | | | 150 pM | | |
|---|---|---|---|---|---|---|
| | % remaining | % remaining | | % remaining | % remaining | |
| | mRNA | mRNA | | mRNA | mRNA | |
| | (mTTR/ | (mTTR/ | | (mTTR/ | (mTTR/ | |
| siRNA | mHPRT) − 1 | mHPRT) − 2 | Mean | mHPRT) − 1 | mHPRT) − 2 | Mean |
| DSR- | 37.51 | 17.76 | 27.63 | 62.55 | 61.23 | 61.89 |
| 0104195 | | | | | | |
| DSR- | 58.55 | 36.88 | 47.71 | 87.97 | 77.66 | 82.82 |
| 0104194 | | | | | | |
| DSR- | 9.58 | 7.49 | 8.53 | 39.20 | 32.93 | 36.06 |
| 0104193 | | | | | | |
| DSR- | 13.14 | 7.94 | 10.54 | 44.02 | 30.37 | 37.19 |
| 0104192 | | | | | | |
| DSR- | 15.78 | 9.95 | 12.86 | 52.74 | 38.31 | 45.53 |
| 0104191 | | | | | | |
| DSR- | 9.38 | 7.34 | 8.36 | 42.30 | 33.88 | 38.09 |
| 0104190 | | | | | | |
| DSR- | 12.15 | 8.99 | 10.57 | 36.30 | 25.84 | 31.07 |
| 0104189 | | | | | | |
| DSR- | 14.20 | 7.08 | 10.64 | 33.73 | 29.77 | 31.75 |
| 0104188 | | | | | | |
| DSR- | 9.56 | 4.44 | 7.00 | 29.73 | 21.58 | 25.65 |
| 0102626 | | | | | | |
| DSR- | 6.26 | 4.21 | 5.23 | 19.95 | 22.30 | 21.13 |
| 0102524 | | | | | | |

| | 50 pM | | |
|---|---|---|---|
| | % remaining | % remaining | |
| | mRNA | mRNA | |
| | (mTTR/ | (mTTR/ | |
| siRNA | mHPRT) − 1 | mHPRT) − 2 | Mean |
| DSR- | 83.75 | 74.55 | 79.15 |
| 0104195 | | | |
| DSR- | 92.42 | 79.02 | 85.72 |
| 0104194 | | | |
| DSR- | 55.87 | 38.79 | 47.33 |
| 0104193 | | | |
| DSR- | 53.59 | 41.64 | 47.61 |
| 0104192 | | | |
| DSR- | 67.24 | 44.44 | 55.84 |
| 0104191 | | | |
| DSR- | 57.64 | 41.91 | 49.77 |
| 0104190 | | | |
| DSR- | 57.65 | 32.08 | 44.87 |
| 0104189 | | | |
| DSR- | 52.91 | 35.11 | 44.01 |
| 0104188 | | | |
| DSR- | 43.41 | 40.04 | 41.72 |
| 0102626 | | | |
| DSR- | 50.50 | 36.79 | 43.65 |
| 0102524 | | | |

Table 179. shows % mouse TTR mRNA remaining (at 500 pM, 150 pM and 50 pM siRNA treatment) relative to mouse HPRT control. N=2. N.D.: Not determined.

| | 500 pM | | | 150 pM | | |
|---|---|---|---|---|---|---|
| | % remaining | % remaining | | % remaining | % remaining | |
| | mRNA | mRNA | | mRNA | mRNA | |
| | (mTTR/ | (mTTR/ | | (mTTR/ | (mTTR/ | |
| siRNA | mHPRT) − 1 | mHPRT) − 2 | Mean | mHPRT) − 1 | mHPRT) − 2 | Mean |
| DSR- | 10.02 | 5.79 | 7.90 | 33.88 | 29.60 | 31.74 |
| 0104203 | | | | | | |
| DSR- | 99.03 | 82.07 | 90.55 | 107.75 | 95.77 | 101.76 |
| 0104202 | | | | | | |
| DSR- | 13.54 | 5.61 | 9.57 | 39.60 | 33.15 | 36.38 |
| 0104201 | | | | | | |
| DSR- | 13.88 | 7.60 | 10.74 | 46.38 | 32.98 | 39.68 |
| 0104200 | | | | | | |
| DSR- | 6.49 | 7.07 | 6.78 | 17.62 | 19.21 | 18.42 |
| 0104199 | | | | | | |
| DSR- | 11.01 | 5.20 | 8.10 | 25.17 | 21.66 | 23.42 |
| 0104198 | | | | | | |
| DSR- | 18.04 | 12.43 | 15.23 | 31.84 | 30.58 | 31.21 |
| 0104197 | | | | | | |
| DSR- | 12.66 | 5.99 | 9.32 | 26.79 | 24.61 | 25.70 |
| 0104196 | | | | | | |
| DSR- | 9.56 | 4.44 | 7.00 | 29.73 | 21.58 | 25.65 |
| 0102626 | | | | | | |
| DSR- | 6.26 | 4.21 | 5.23 | 19.95 | 22.30 | 21.13 |
| 0102524 | | | | | | |

| | 50 pM | | |
|---|---|---|---|
| | % remaining | % remaining | |
| | mRNA | mRNA | |
| | (mTTR/ | (mTTR/ | |
| siRNA | mHPRT) − 1 | mHPRT) − 2 | Mean |
| DSR- | 52.26 | 41.98 | 47.12 |
| 0104203 | | | |
| DSR- | 88.46 | 99.42 | 93.94 |
| 0104202 | | | |
| DSR- | 50.68 | 44.27 | 47.48 |
| 0104201 | | | |
| DSR- | 53.43 | 52.31 | 52.87 |
| 0104200 | | | |
| DSR- | 51.46 | 36.42 | 43.94 |
| 0104199 | | | |
| DSR- | 48.50 | 29.30 | 38.90 |
| 0104198 | | | |
| DSR- | 64.28 | 49.15 | 56.72 |
| 0104197 | | | |
| DSR- | 49.82 | 36.49 | 43.16 |
| 0104196 | | | |
| DSR- | 43.41 | 40.04 | 41.72 |
| 0102626 | | | |
| DSR- | 50.50 | 36.79 | 43.65 |
| 0102524 | | | |

Table 180. shows % mouse TTR mRNA remaining (at 500 pM, 150 pM and 50 pM siRNA treatment) relative to mouse HPRT control. N=2. N.D.: Not determined.

| | 500 pM | | | 150 pM | | |
|---|---|---|---|---|---|---|
| | % remaining | % remaining | | % remaining | % remaining | |
| | mRNA | mRNA | | mRNA | mRNA | |
| | (mTTR/ | (mTTR/ | | (mTTR/ | (mTTR/ | |
| siRNA | mHPRT) − 1 | mHPRT) − 2 | Mean | mHPRT) − 1 | mHPRT) − 2 | Mean |
| DSR- | 14.51 | 10.61 | 12.56 | 32.18 | 41.17 | 36.67 |
| 0104211 | | | | | | |
| DSR- | 30.92 | 23.80 | 27.36 | 55.84 | 75.52 | 65.68 |
| 0104210 | | | | | | |
| DSR- | 19.60 | 11.60 | 15.60 | 48.47 | 51.32 | 49.90 |
| 0104208 | | | | | | |
| DSR- | 19.64 | 11.12 | 15.38 | 50.40 | 48.19 | 49.30 |
| 0104207 | | | | | | |
| DSR- | 14.04 | 7.77 | 10.90 | 46.65 | 40.12 | 43.38 |
| 0104206 | | | | | | |
| DSR- | 9.84 | 6.34 | 8.09 | 39.68 | 33.06 | 36.37 |
| 0104205 | | | | | | |
| DSR- | 11.09 | 6.75 | 8.92 | 44.00 | 29.73 | 36.87 |
| 0104204 | | | | | | |
| DSR- | 9.56 | 4.44 | 7.00 | 29.73 | 21.58 | 25.65 |
| 0102626 | | | | | | |
| DSR- | 6.26 | 4.21 | 5.23 | 19.95 | 22.30 | 21.13 |
| 0102524 | | | | | | |

| | 50 pM | | |
|---|---|---|---|
| | % remaining | % remaining | |
| | mRNA | mRNA | |
| | (mTTR/ | (mTTR/ | |
| siRNA | mHPRT) − 1 | mHPRT) − 2 | Mean |
| DSR- | 65.09 | 57.10 | 61.09 |
| 0104211 | | | |
| DSR- | 81.88 | 68.36 | 75.12 |
| 0104210 | | | |
| DSR- | 70.93 | 59.49 | 65.21 |
| 0104208 | | | |
| DSR- | 66.28 | 60.44 | 63.36 |
| 0104207 | | | |
| DSR- | 55.19 | 46.36 | 50.78 |
| 0104206 | | | |
| DSR- | 58.92 | 36.51 | 47.72 |
| 0104205 | | | |
| DSR- | 55.79 | 47.81 | 51.80 |
| 0104204 | | | |
| DSR- | 43.41 | 40.04 | 41.72 |
| 0102626 | | | |
| DSR- | 50.50 | 36.79 | 43.65 |
| 0102524 | | | |

### Example 17. Provided Oligonucleotides and Compositions are Active In Vitro in Human iCell GABA Neurons

Various siRNAs for mouse APP were designed and constructed. A number of siRNAs were tested in vitro in human iCell GABA neurons at one or a range of concentrations. Some siRNAs were also tested in mice (e.g., C57BL6 wild type mice).

Example protocol for in vitro determination of siRNA activity in human iCell GABA neurons: For determination of siRNAs activity, siRNAs at specific concentration were gymnotically delivered to human iCell GABA neurons plated at 96-well plates, with 40,000 cells/well. Following 5 days treatment, total RNA was extracted using SV96 Total RNA Isolation kit (Promega). cDNA production from RNA samples were performed using High-Capacity cDNA Reverse Transcription kit (Thermo Fisher) following manufacturer's instructions and qPCR analysis performed in CFX System using iQ Multiplex Powermix (Bio-Rad). For human APP mRNA, the following qPCR assay were utilized: Thermofisher Taqman qPCR assay ID Hs00169098_m1. Human SFRS9 was used as normalizer (Forward, 5′-TGGAATATGCCCTGCGTAAA-3′; Reverse, 5′-TGGTGCTTCTCTCAGGATAAAC-3′, Probe, 5′-TGGATGACACCAAATTCCGCTCTCA-3′. mRNA knockdown levels were calculated as % mRNA remaining relative to mock treatment.

Table 181. shows % human APP mRNA remaining (at 3 and 1 uM siRNA treatment) in iCell GABA neurons relative to human SFRS9 control. N=2. N.D.: Not determined.

| | 3 uM | | | 1 uM | | |
|---|---|---|---|---|---|---|
| | % remaining | % remaining | | % remaining | % remaining | |
| | mRNA | mRNA | | mRNA | mRNA | |
| | (hAPP/ | (hAPP/ | | (hAPP/ | (hAPP/ | |
| siRNA | hSFRS9) − 1 | hSFRS9) − 2 | Mean | hSFRS9) − 1 | hSFRS9) − 2 | Mean |
| DSR- | 15.80 | 13.20 | 14.50 | 26.70 | 21.60 | 24.15 |
| 0104213 | | | | | | |
| DSR- | 19.60 | 18.70 | 19.15 | 34.90 | 42.60 | 38.75 |
| 0104223 | | | | | | |
| DSR- | 18.30 | 17.90 | 18.10 | 31.10 | 30.80 | 30.95 |
| 0104222 | | | | | | |
| DSR- | 64.10 | 62.10 | 63.10 | 79.00 | 74.00 | 76.50 |
| 0104221 | | | | | | |
| DSR- | 39.40 | 37.80 | 38.60 | 81.70 | 65.70 | 73.70 |
| 0104220 | | | | | | |
| DSR- | 28.70 | 32.10 | 30.40 | 55.70 | 58.80 | 57.25 |
| 0104219 | | | | | | |
| DSR- | 52.20 | 61.80 | 57.00 | 74.30 | 78.00 | 76.15 |
| 0104218 | | | | | | |
| DSR- | 40.40 | 40.10 | 40.25 | 66.40 | 63.50 | 64.95 |
| 0104217 | | | | | | |
| DSR- | 51.20 | 57.10 | 54.15 | 60.10 | 63.10 | 61.60 |
| 0104216 | | | | | | |
| DSR- | 43.40 | 42.10 | 42.75 | 65.90 | 54.90 | 60.40 |
| 0104215 | | | | | | |
| DSR- | 19.60 | 19.40 | 19.50 | 34.60 | 31.00 | 32.80 |
| 0104214 | | | | | | |

Table 182. shows % human APP mRNA remaining (at 3 and 1 uM siRNA treatment) in iCell GABA neurons relative to human SFRS9 control. N=2. N.D.: Not determined.

| | 3 uM | | | 1 uM | | |
|---|---|---|---|---|---|---|
| | % remaining | % remaining | | % remaining | % remaining | |
| | mRNA | mRNA | | mRNA | mRNA | |
| | (hAPP/ | (hAPP/ | | (hAPP/ | (hAPP/ | |
| siRNA | hSFRS9) − 1 | hSFRS9) − 2 | Mean | hSFRS9) − 1 | hSFRS9) − 2 | Mean |
| DSR- | 15.80 | 13.20 | 14.50 | 26.70 | 21.60 | 24.15 |
| 0104213 | | | | | | |
| DSR- | 9.20 | 9.10 | 9.15 | 27.00 | 8.30 | 17.65 |
| 0104231 | | | | | | |
| DSR- | 18.90 | 18.90 | 18.90 | 32.20 | 32.60 | 32.40 |
| 0104230 | | | | | | |
| DSR- | 13.80 | 11.70 | 12.75 | 31.70 | 31.10 | 31.40 |
| 0104229 | | | | | | |
| DSR- | 12.20 | 12.70 | 12.45 | 26.10 | 25.60 | 25.85 |
| 0104228 | | | | | | |
| DSR- | 19.20 | 20.60 | 19.90 | 36.80 | 39.90 | 38.35 |
| 0104227 | | | | | | |
| DSR- | 13.30 | 12.80 | 13.05 | 30.00 | 27.40 | 28.70 |
| 0104226 | | | | | | |
| DSR- | 32.30 | 34.70 | 33.50 | 57.60 | 56.80 | 57.20 |
| 0104225 | | | | | | |
| DSR- | 55.10 | 62.80 | 58.95 | 65.60 | 81.40 | 73.50 |
| 0104224 | | | | | | |

Table 183. shows % human APP mRNA remaining (at 3 and 1 uM siRNA treatment) in iCell GABA neurons relative to human SFRS9 control. N=2. N.D.: Not determined.

| | 3 uM | | | 1 uM | | |
|---|---|---|---|---|---|---|
| | % remaining | % remaining | | % remaining | % remaining | |
| | mRNA | mRNA | | mRNA | mRNA | |
| | (hAPP/ | (hAPP/ | | (hAPP/ | (hAPP/ | |
| siRNA | hSFRS9) − 1 | hSFRS9) − 2 | Mean | hSFRS9) − 1 | hSFRS9) − 2 | Mean |
| DSR- | 15.80 | 13.20 | 14.50 | 26.70 | 21.60 | 24.15 |
| 0104213 | | | | | | |
| DSR- | 18.30 | 18.70 | 18.50 | 43.40 | 51.00 | 47.20 |
| 0104233 | | | | | | |
| DSR- | 15.70 | 15.50 | 15.60 | 21.70 | 28.10 | 24.90 |
| 0104232 | | | | | | |
| DSR- | 11.70 | 10.90 | 11.30 | 28.10 | 27.90 | 28.00 |
| 0103301 | | | | | | |
| DSR- | 32.10 | 31.20 | 31.65 | 62.90 | 57.10 | 60.00 |
| 0104241 | | | | | | |
| DSR- | 50.50 | 47.30 | 48.90 | 64.70 | 76.10 | 70.40 |
| 0104240 | | | | | | |
| DSR- | 38.60 | 39.40 | 39.00 | 55.00 | 61.80 | 58.40 |
| 0104239 | | | | | | |
| DSR- | 25.80 | 26.30 | 26.05 | 55.70 | 56.70 | 56.20 |
| 0104238 | | | | | | |
| DSR- | 25.40 | 21.50 | 23.45 | 50.20 | 55.20 | 52.70 |
| 0104237 | | | | | | |
| DSR- | 24.80 | 24.80 | 24.80 | 61.40 | 66.70 | 64.05 |
| 0104236 | | | | | | |
| DSR- | 29.00 | 28.60 | 28.80 | 58.60 | 62.70 | 60.65 |
| 0104235 | | | | | | |
| DSR- | 21.70 | 23.70 | 22.70 | 51.90 | 59.60 | 55.75 |
| 0104234 | | | | | | |

### Example 18: Synthesis and Deprotection of Stereopure Oligonucleotide Sequences Containing 5′-Phosphonate

### Abbreviation

ETT: 5-(Ethylthio)-1H-tetrazole
CMIMT: N-cyanomethylimidazolium triflate
ADIH: 2-azido-1,3-dimethylimidazolium hexafluorophosphate
CPG: controlled pore glass
DCM: dichloromethane, CH₂Cl₂
ACN: acetonitrile
IBN: isobutyronitrile
DMSO: Dimethyl sulfoxide
MeIm: N-methylimidazole
TCA: trichloroacetic acid
TEA: triethylamine
TEA-3HF: triethylamine trihydrofluoride
DS1 reagent: TEA-3HF:TEA:H₂O:DMSO=5.0:7.0:14.7:73.3 (v/v/v/v)
TMSI: Trimethylsilyl iodide
XH: xanthane hydride
Ac₂O: acetic anhydride
Vped5m: 5-(E)-vinylphosphonate-5-deoxy-5methyl

### General Procedure for the Synthesis of Chiral-Oligos (25 μMol Scale)

The automated solid-phase synthesis of chiral-oligos was performed according to the cycles shown in Table 1 (regular amidite cycle, for PO linkages), Table 2 (DPSE amidite cycle using 2′-deoxy-5′-phosphonate nucleosides, for chiral PS linkages), Table 3 (PSM amidite cycle using 2′-O-methyl and 2′-O-methoxyethyl nucleosides, for chiral PS linkages), Table 4 (PSM amidite cycle, for chiral P^{N} linkages). All the amidites were dissolved into the appropriate solvents (Acetonitrile or 20% isobutyronitrile in 80% acetonitrile), dried at least for 2 h under 4A molecular sieves and then was used in the synthetic cycle. After the last coupling of 5′-phosphonate amidite, the column was washed with 20% DEA in acetonitrile.

TABLE 184
Regular Amidite Synthetic Cycle for PO linkages

| | | | | waiting |
|---|---|---|---|---|
| step | operation | reagents and solvent | volume | time |
| 1 | detritylation | 3% TCA/DCM | 10 mL | 65 s |
| 2 | coupling | 0.2M monomer/20% IBN- | 0.5 mL | 8 min |
| | | MeCN 0.5M ETT/MeCN | 1.0 mL | |
| 3 | oxidation | 50 mM I₂/pyridine-H₂O (9:1, | 2.0 mL | 1 min |
| | | v/v) | | |
| 4 | cap-2 | 20% Ac₂O, 30% 2,6-lutidine/ | 1.0 mL | 45 s |
| | | MeCN 20% MeIm/MeCN | 1.0 mL | |

TABLE 185
DPSE Amidite Synthetic Cycle for chiral PS linkages

| | | | | waiting |
|---|---|---|---|---|
| step | operation | reagents and solvent | volume | time |
| 1 | detritylation | 3% TCA/DCM | 10 mL | 65 s |
| 2 | coupling | 0.2M monomer/20% IBN- | 0.5 mL | 8 min |
| | | MeCN 0.5M CMIMT/MeCN | 1.0 mL | |
| 3 | cap-1 | 20% Ac₂O, 30% 2,6-lutidine/ | 2.0 mL | 2 min |
| | | MeCN | | |
| 4 | sulfurization | 0.2M XH/pyridine | 2.0 mL | 6 min |
| 5 | cap-2 | 20% Ac₂O, 30% 2,6-lutidine/ | 1.0 mL | 45 s |
| | | MeCN 20% MeIm/MeCN | 1.0 mL | |

TABLE 186
PSM Amidite Synthetic Cycle for chiral PS linkages

| | | | | waiting |
|---|---|---|---|---|
| step | operation | reagents and solvent | volume | time |
| 1 | detritylation | 3% TCA/DCM | 10 mL | 65 s |
| 2 | coupling | 0.2M monomer/20% IBN- | 0.5 mL | 8 min |
| | | MeCN 0.5M CMIMT/MeCN | 1.0 mL | |
| 3 | cap-1 | 20% Ac₂O, 30% 2,6-lutidine/ | 2.0 mL | 2 min |
| | | MeCN | | |
| 4 | sulfurization | 0.2M XH/pyridine | 2.0 mL | 6 min |
| 5 | cap-2 | 20% Ac₂O, 30% 2,6-lutidine/ | 1.0 mL | 45 s |
| | | MeCN 20% MeIm/MeCN | 1.0 mL | |

TABLE 187
PSM Amidite Synthetic Cycle for chiral PN linkages (n001)

| | | | | waiting |
|---|---|---|---|---|
| step | operation | reagents and solvent | volume | time |
| 1 | detritylation | 3% TCA/DCM | 10 mL | 65 s |
| 2 | coupling | 0.2M monomer/20% IBN- | 0.5 mL | 8 min |
| | | MeCN 0.5M CMIMT/MeCN | 1.0 mL | |
| 3 | Imidation | 0.5M ADIH/MeCN | 2.0 mL | 6 min |
| 4 | cap-2 | 20% Ac₂O, 30% 2,6-lutidine/ | 1.0 mL | 45 s |
| | | MeCN 20% MeIm/MeCN | 1.0 mL | |

### General Procedure for the 5′-phosphonate Deprotection Conditions (25 μmole)

To prepare TMSI solution for 5′-phosphonate deprotection, pyridine (0.5 mL) was added to DCM (23.9 mL) and the resulting solution was cooled in ice-bath for 15 minutes. After that TMSI reagent (0.6 mL) was added to the mixture to get a bright yellow solution (total volume 25.0 mL).

TMSI quenching solution (50 mL) was prepared by adding 2-Dodecane thiol (12.0 mL) and TEA (18.0 mL) in acetonitrile (18.0 mL).

After completion of the synthesis, the CPG solid support was dried and transferred into 50 mL plastic tube. Minimum amount of DCM was added to 5′-Phosphonate containing oligonucleotide on CPG and the CPG was vortexed to get a homogenous slurry. To this homogenous slurry, the TMSI solution (10.0 mL) was added slowly and mixed well. After the addition of total TMSI solution the color of the reaction mixture turns yellow indicating excess of TMSI solution. The resulting reaction mixture was stirred for 30 minutes at room temperature. After 30 min. the support was promptly washed using excess of acetonitrile followed by addition of quenching solution (10.0 mL) and this process was repeated three times (Total quenching volume 30.0 mL). The total time of exposure was limited to 20 min. The CPG was rinsed thoroughly using acetonitrile and drying under vacuum. Afterwards, CPG was subjected to standard cleavage and deprotection condition.

### General Procedure for the C&D Conditions (25 μMol Scale)

After completion of the synthesis and 5′-phosphonate deprotection, the CPG solid support was dried and transferred into 50 mL plastic tube. The CPG was treated with DS1 reagent (2.5 mL; 100 uL/umol) for 3 h at 27° C., then added conc. NH₃ (5.0 mL; 200 umol/umol) for 24 h at 37° C. The reaction mixture was cooled to room temperature and the CPG was separated by membrane filtration, washed with 15 mL of H₂O. The crude material (filtrate) was analyzed by LTQ and RP-UPLC.

| | Crude ODs/μmol | calcd. [M] | found [M] | UPLC purity |
|---|---|---|---|---|
| SSR-0106222 | 64.94 O.D./μmole | 8014.8 | 8013.2 | 25.95% |
| SSR-0106517 | 34.0 O.D./μmole | 7998.8 | 7998.9 | 25.60% |

### Example 19: Synthesis of 5′-PO(OEt)₂ Vinyl Phosphonate-dT (WV-NU-017)

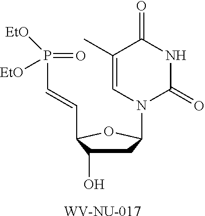

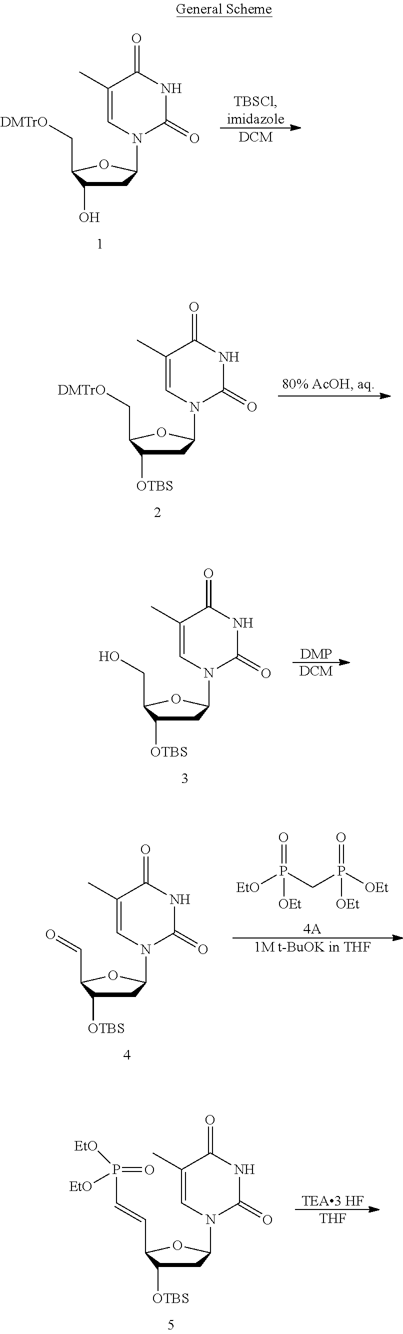

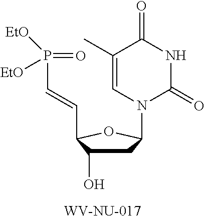

### 1. Preparation of Compound 2

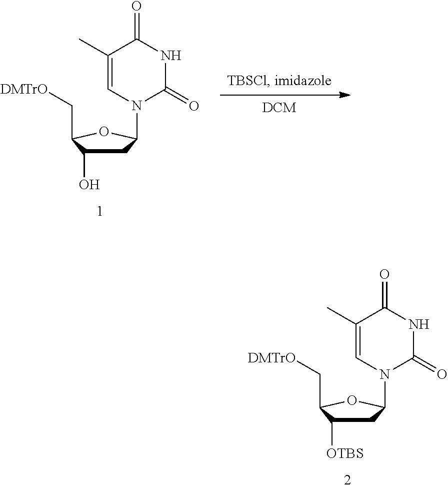

Two batches: To a solution of compound 1 (150 g, 275.43 mmol) and imidazole (56.25 g, 826.30 mmol) in DCM (2 L) was added TBSCl (83.03 g, 550.87 mmol, 67.50 mL). The mixture was stirred at 15° C. for 16 hr. TLC showed compound 1 was consumed. Two batches: The mixture was washed with sat. NaHCO₃ (aq., 2 L * 2), the combined aqueous was extracted with EtOAc (500 mL*2), the combined organic was dried over Na₂SO₄, filtered and concentrated to give crude. Compound 2 (362 g, crude) was obtained as a yellow oil.

TLC (Petroleum ether/Ethyl acetate=1:1, 5% TEA) R_{f}=0.39.

### 2. Preparation of Compound 3

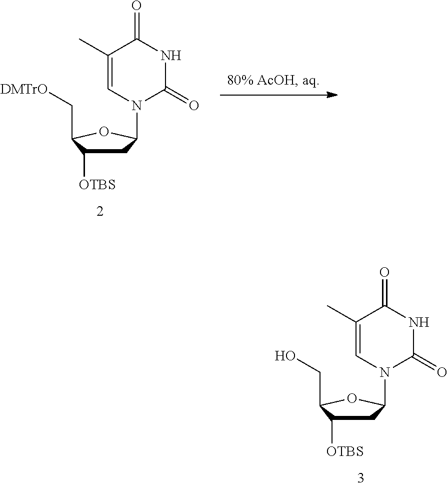

A solution of compound 2 (362 g, 549.44 mmol) in H₂O (360 mL) and CH₃COOH (1440 mL), the mixture was stirred at 15° C. for 16 hr. TLC showed compound 2 was consumed. The mixture was added sat. NaHCO₃ (aq., 3000 mL), and the organic layer was separated and the aqueous layer extracted with EtOAc (2000 mL*3), the combined organic was dried over Na₂SO₄, filtered and concentrated to get the crude. The residue was purified by silica gel chromatography (SiO₂, Petroleum ether:Ethyl acetate=30:1 to 1:2) to give compound 3 (185 g, 94.45% yield) was obtained as a white solid.

TLC (Petroleum ether:Ethyl acetate=1:1) Rf=0.24.

### 3. Preparation of Compound 4

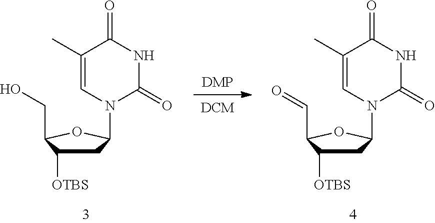

To a solution of compound 3 (110 g, 308.57 mmol) in DCM (500 mL) was added DMP (157.05 g, 370.28 mmol, 114.64 mL) in portions at 0° C. The mixture was stirred at 30° C. for 2 hr. TLC showed most of compound 3 was disappeared and a new spot was found. Two batches: The mixture was added 5% Na₂S₂O₃ (2000 mL) at 0° C., the mixture was stirred at 0° C. for 20 min then sat. NaHCO₃ (2000 mL) was added, the mixture was extracted with DCM (2000 mL*4) and Ethyl acetate (2000 mL*4), the combined organic was dried over Na₂SO₄, filtered, and concentrated to get compound 4 (190 g, crude) was obtained as a white solid.

TLC (Petroleum ether:Ethyl acetate=1:3) R_{f}=0.36.

### 4. Preparation of Compound 5

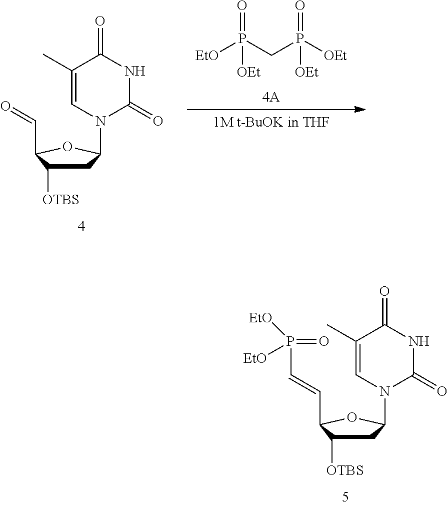

To a solution of compound 4A (216.28 g, 750.41 mmol) in THF (400 mL) was added t-BuOK (1 M, 750 mL) at 0° C. and stirred at 0° C. for 10 min, then warmed up to 20° C. for 2 hr. The above mixture was added to the solution of compound 4 (190 g, 536.01 mmol) in THF (400 mL) at 0° C. The reaction mixture was stirred at 0° C. for 1 hr and then allowed to warm up to 20° C. in 6 hr. TLC showed the reaction was complete. To the reaction mixture water (1000 mL) was added and extracted with EtOAc (2000 mL*4) and DCM (1000 mL*3). The organic phase was dried over Na₂SO₄, filtered, and concentrated to give residue. The residue was purified by column chromatography (SiO₂, Petroleum ether:Ethyl acetate=10:1 1:1, 0:1) to give compound 5 (250 g, crude) was obtained as a yellow oil.

TLC (Petroleum ether/Ethyl acetate=1:2), R_{f}=0.32.

### 5. Preparation of WV-NU-017

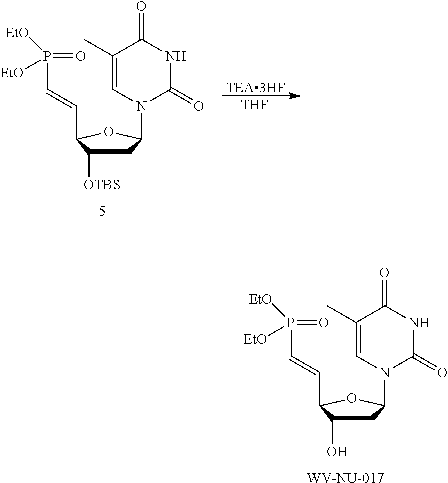

To a solution of compound 5 (243 g, 497.35 mmol) in THF (1300 mL) was added TEA.3HF (320.71 g, 1.99 mol, 324.28 mL). The mixture was stirred at 15° C. for 16 hr. TLC (Petroleum ether: (Ethyl acetate: Ethyl Alcohol=3:1)=1:1, R_{f}=0.18) showed a few of compound 5 was remained and new spot was detected. The reaction mixture was concentrated under reduced pressure and the mixture was neutralized with Na₂CO₃ (aq., sat., ˜1 L) until pH=7. The mixture was concentrated under reduced pressure to removed most of water. The mixture was added EtOAc:EtOH=10:1 (1 L * 2) and dried over Na₂SO₄, filtered and concentrated under reduced pressure to give a residue. The residue was purified by column chromatography (SiO₂, (Ethyl acetate:Ethyl Alcohol=3:1)/Petroleum ether=5%, 10%, 15%, 20%, 25%, 30%, 40%, 50%) to give compound WV-NU-017 (76 g, 38.51% yield) was obtained as a white solid.

¹H NMR (400 MHz, CDCl₃) δ=9.59 (br s, 1H), 7.13 (d, J=1.1 Hz, 1H), 7.09-6.94 (m, 1H), 6.41 (t, J=6.6 Hz, 1H), 6.03 (ddd, J=1.8, 17.4, 19.6 Hz, 1H), 4.76 (br d, J=4.0 Hz, 1H), 4.50 (br d, J=2.4 Hz, 1H), 4.39 (br d, J=2.0 Hz, 1H), 4.21-4.00 (m, 4H), 2.43 (ddd, J=4.5, 6.3, 13.8 Hz, 1H), 2.18 (td, J=6.9, 13.8 Hz, 1H), 2.02-1.86 (m, 3H), 1.38-1.30 (m, 6H).

³¹P NMR (162 MHz, CDCl₃) δ=17.71.

¹³C NMR (101 MHz, CDCl₃) δ=163.76, 150.63, 149.04, 135.29, 118.47, 116.58, 111.67, 85.87, 85.65, 85.07, 73.91, 62.23, 39.15, 16.38, 12.66.

LCMS (M−H⁺): 373.1, purity: 94.34%.

TLC (Petroleum ether: (Ethyl acetate: Ethyl Alcohol=3:1)=1:1) R_{f}=0.18;

### Example 20: Synthesis of 5′-PO(OMe)₂ Vinyl Phosphonate-dT (WV-NU-010), and 5′-PO(OMe)₂ Vinyl Phosphonate-3′-CNE-dT Phosphoramidite (WV-NU-10-CNE)

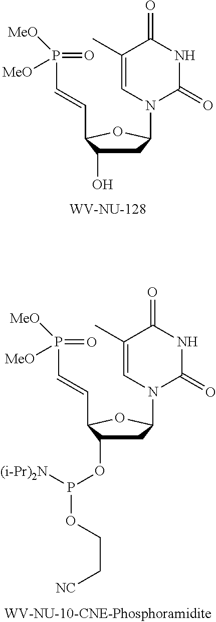

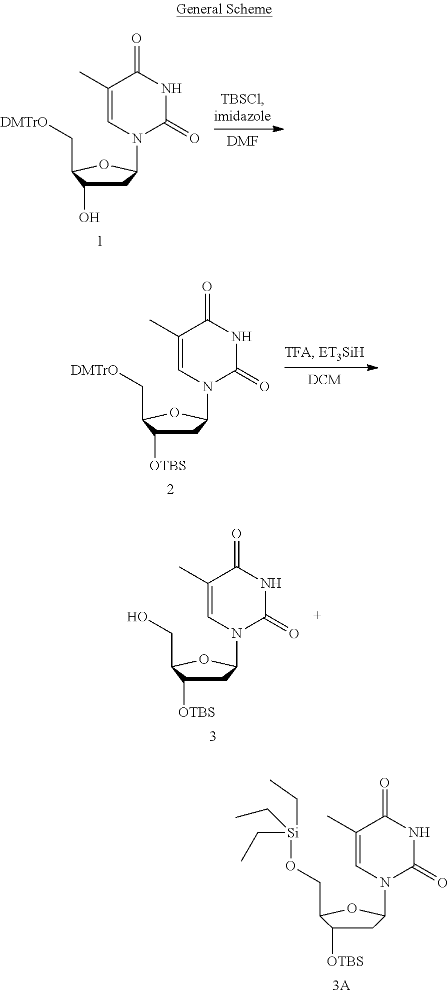

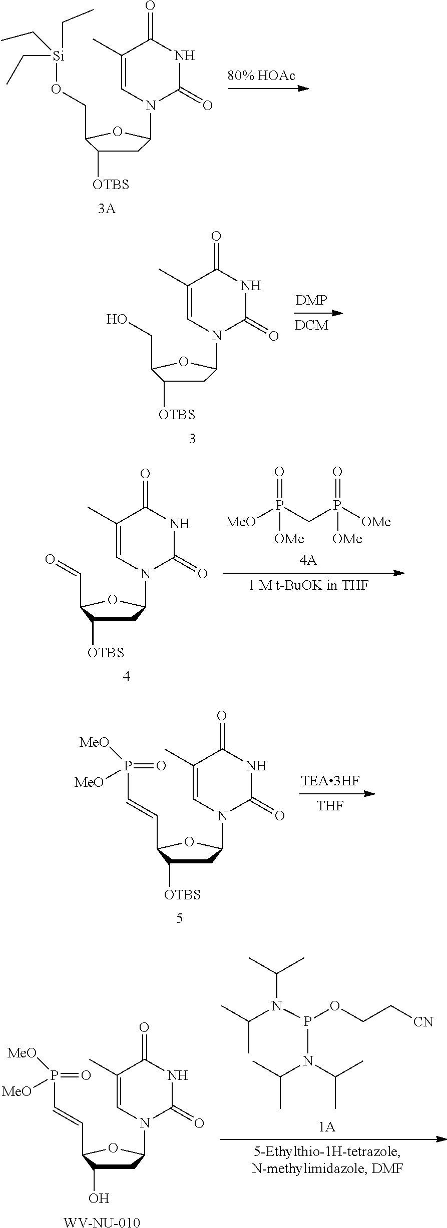

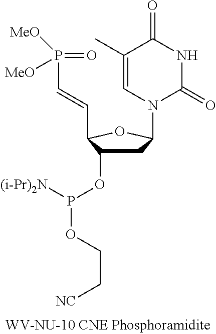

### 2. Preparation of Compound 2

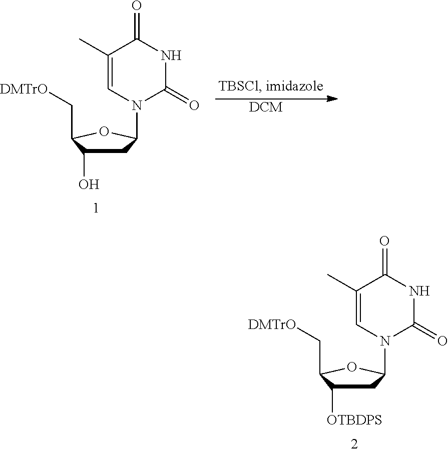

To a solution of compound 1 (100.00 g, 183.62 mmol) and imidazole (37.50 g, 550.86 mmol) in DCM (1.00 L) was added TBSCl (55.35 g, 367.24 mmol) at 0° C., and the mixture was stirred at 18° C. for 14 hr. TLC showed the starting material was consumed. The mixture was washed with sat. NaHCO₃ (200 mL) and brine (100 mL), dried over Na₂SO₄, filtered and concentrated to get the compound 2 as a white solid (120.98 g, crude). The mixture was used for next step directly without any purification.

TLC (Ethyl acetate/Petroleum ether=3:1, 5% TEA) Rf=0.43.

### 2. Preparation of Compound 3 & Compound 3A

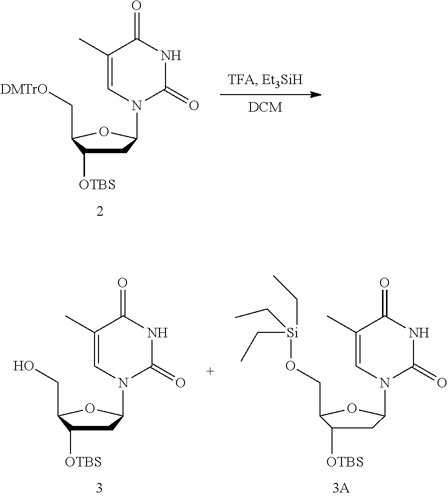

To a solution of TFA (41.85 g, 367.00 mmol) and Et₃SiH (64.01 g, 550.50 mmol) in DCM (1.20 L) was added compound 2 (120.9 g, 183.50 mmol) dissolved in DCM (200.00 mL), and the mixture was stirred at 15° C. for 0.5 h. TLC showed the starting material was consumed. The mixture was added sat. NaHCO₃ (aq, 300 mL), and the organic phase was separated and the aqueous layer extracted with DCM (200 mL*3), the combined organic was dried over Na₂SO₄, filtered and concentrated to get the crude. The residue was purified by MPLC (Petroleum ether/Ethyl acetate=10:1 to 1:2) to get compound 3 as a white solid (36 g, 55.04% yield) and compound 3A as a white solid (50 g, crude).

¹H NMR (400 MHz, CDCl₃) δ=9.08 (s, 1H), 6.04 (t, J=6.8 Hz, 1H), 4.37 (td, J=3.5, 6.5 Hz, 1H), 3.84-3.74 (m, 2H), 3.67-3.58 (m, 1H), 2.22 (td, J=6.8, 13.4 Hz, 1H), 2.13-2.03 (m, 1H), 1.78 (s, 3H), 0.81-0.77 (m, 3H), 0.77 (s, 9H), −0.04 (s, 6H);

TLC (Petroleum ether/Ethyl acetate=1:1) Rf=0.24.

### 3. Preparation of Compound 3

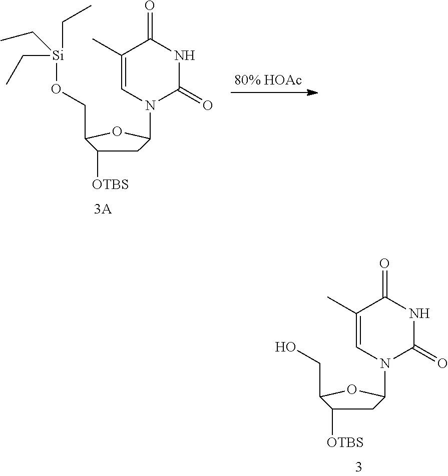 [SMILES: CC[Si](CC)(CC)OCC1OC(n2cc(C)c(=O)[nH]c2=O)CC1O[Si](C)(C)C(C)(C)C ; Cc1cn(C2CC(O[Si](C)(C)C(C)(C)C)C(CO)O2)c(=O)[nH]c1=O]

To a solution of compound 3A (50 g, 106.21 mmol) in the mixture of HOAc (210.00 g, 3.50 mol) and H₂O (50 mL), the mixture was at 18° C. for 0.5 h. TLC showed the starting material was consumed. The mixture was added Na₂CO₃ (aq) until PH>8, and the residue was extracted the EtOAc (200 mL*3). The mixture was purified by MPLC (Petroleum ether/Ethyl acetate=10:1, 1:1) to get compound 3 as a white solid (30 g, 79.23% yield).

¹H NMR (400 MHz, CDCl₃) δ=8.98 (br s, 1H), 7.38 (s, 1H), 6.15 (t, J=6.8 Hz, 1H), 4.50 (td, J=3.5, 6.6 Hz, 1H), 3.96-3.88 (m, 2H), 3.81-3.67 (m, 1H), 2.81-2.69 (m, 1H), 2.39-2.17 (m, 2H), 1.91 (s, 3H), 0.99-0.84 (m, 9H), 0.09 (s, 6H).

### 4. Preparation of Compound 4

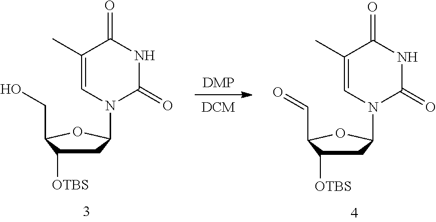

To a solution of compound 3 (10 g, 28.05 mmol) in DCM (160 mL) was added DMP (14.28 g, 33.66 mmol, 10.42 mL) at 0° C. The mixture was stirred at 0-25° C. for 3 hr. TL C showed most of the starting material was disappeared and a new spot was found. The mixture was added 5% Na₂S₂O₃ (300 mL) at 0° C., the mixture was stirred at 0° C. for 20 min then sat. NaHCO₃ (300 mL) was added, the mixture was extracted with DCM (200 mL*3), the combined organic was dried over Na₂SO₄, filtered and concentrated to get the compound 4 as a yellow oil (10 g, crude). The mixture was used for next step without any purification.

TL C (Petroleum ether/Ethyl acetate=1:1) Rf=0.38.

### 5. Preparation of Compound 5

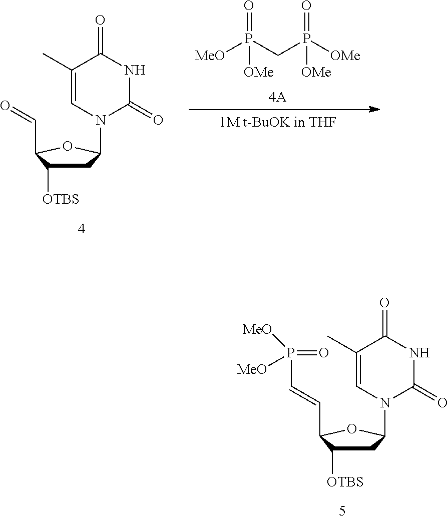

To a solution of compound 4A (7.20 g, 31.03 mmol) in THF (20 mL) was added t-BuOK (1 M, 31.03 mL) at 0° C. and stirred at 0° C. for 10 min, then warmed up to 20° C. for 30 min. The above mixture was added to the solution of compound 4 (10 g, 28.21 mmol) in THF (20 mL) at 0° C. The reaction mixture was stirred at 0° C. for 1 h and then allowed to warm up to 20° C. in 20 min. LCMS and TLC showed the reaction was complete. To the reaction mixture water (20 mL) was added to the reaction and extracted with EtOAc (30 mL*4). The organic phase was dried (Na₂SO₄), filtered and concentrated. The residue was purified by column chromatography MPLC (Petroleum ether/Ethyl acetate=10:1 to 1:4) to get compound 5 as a white solid (12 g, 92.36% yield). The mixture of 10 g crude was purified with the product together.

¹H NMR (400 MHz, CDCl₃) δ=8.82 (br s, 1H), 7.09 (s, 1H), 6.94-6.78 (m, 1H), 6.33 (t, J=6.7 Hz, 1H), 5.99 (ddd, J=1.6, 17.4, 19.2 Hz, 1H), 4.41-4.24 (m, 2H), 3.76 (dd, J=3.8, 11.1 Hz, 5H), 2.33-2.23 (m, 1H), 2.13 (td, J=6.8, 13.6 Hz, 1H), 1.96-1.89 (m, 3H), 0.94-0.80 (m, 10H), 0.14-0.03 (m, 6H);

TLC (Dichloromethane:Methanol=20:1) Rf=0.36.

### 6. Preparation of WV-NU-010

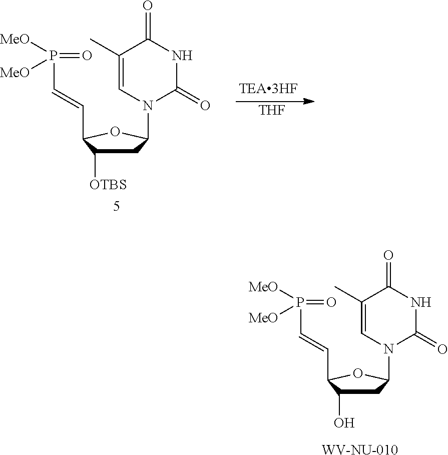

To a solution of compound 5 (13 g, 28.23 mmol) in THF (80 mL) was added N, N-diethylethanamine; trihydrofluoride (22.75 g, 141.14 mmol). The mixture was stirred at 18° C. for 12 hour. TLC showed a few of the starting material was remained and the desired substance was found. The reaction mixture was concentrated under reduced pressure and the mixture was neutralised with Na₂CO₃ (aq. sat) until pH=7. The water phase was freeze-dried. The freeze-drying solid was washed with DCM:MeOH=10:1 (300 mL*2). The organic phase was concentrated. The residue obtained was purified by column chromatography on silica gel (Dichloromethane:Methanol=100:1, 100:8) to get WV—NU-010 as a white solid (6.25 g, 62.54% yield). The mixture was purified with another batch (8 g scale). We got the WV-NU-010 10 g as a white solid in total.

¹H NMR: (400 MHz, CDCl₃) δ=9.54 (br s, 1H), 7.13-6.98 (m, 2H), 6.39 (t, J=6.7 Hz, 1H), 6.06-5.96 (m, 1H), 4.61 (br d, J=4.0 Hz, 1H), 4.50 (br d, J=2.4 Hz, 1H), 4.44-4.36 (m, 1H), 3.79-3.71 (m, 6H), 2.43 (ddd, J=4.5, 6.4, 13.8 Hz, 1H), 2.21 (td, J=6.9, 13.7 Hz, 1H), 1.93 (s, 3H);

³¹PNMR: (162 MHz, CDCl₃): δ=20.54 (s, 1P);

LCMS: (M+H⁺): 347.0 LCMS purity: 97.81%;

¹³CNMR: (101 MHz, CDCl₃) δ=163.95, 150.75, 150.12, 150.06, 135.38, 116.99, 115.10, 111.65, 85.82, 85.61, 85.14, 73.93, 52.69, 39.00, 12.61;

HPLC: HPLC purity: 98.25%;

TLC (Dichloromethane/Methanol) Rf=0.24.

### 7. Preparation of WV-NU-10-CNE-Phosphoramidite

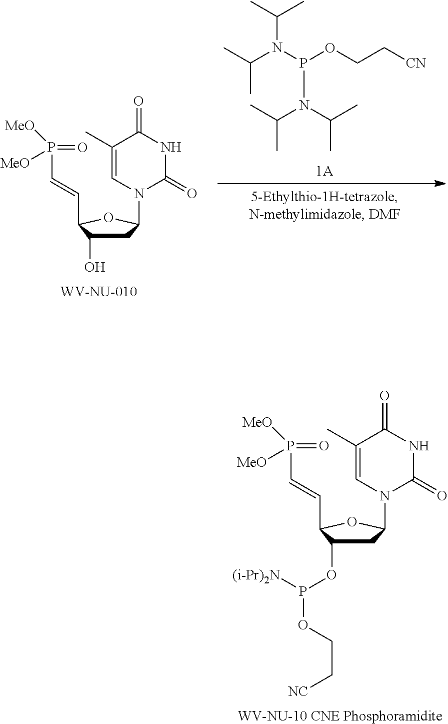

WV-NU-010 (4.9 g, 14.15 mmol) was co-evaporated with anhydrous toluene two times (25 mL×2) and dried under high vacuum for 1 h.

To a solution of WV-NU-010 (4.9 g, 14.15 mmol) in DMF (35 mL) was added 5-ethylsulfanyl-2H-tetrazole (1.84 g, 14.15 mmol), 1-methylimidazole (2.32 g, 28.30 mmol) and compound 1A (6.40 g, 21.23 mmol). The reaction mixture was stirred at 20° C. under N₂ for 1 h. TLC showed the starting material was consumed and the desired substance was found. The reaction mixture was diluted with EtOAc (60 mL). The reaction mixture was washed with aq. saturated. NaHCO₃ solution (50 mL*4), dried over Na₂SO₄, filtered and concentrated under reduced pressure. The column was eluted with Petroleum ether/Ethyl acetate (5% TEA 10 mins) and then Petroleum ether (5 mins). The residue thus obtained was purified by silica gel column chromatography (elution with Petroleum ether:EtOAc=10:1, 1:1 and then EtOAc/Acetonitrile=50:1, 30:1) to get WV-NU-10-CNE-Phosphoramidite as a white solid (4.4 g, 54.14% yield).

¹H NMR: (400 MHz, CDCl₃) δ=8.96 (br s, 1H), 7.08 (s, 1H), 7.03-6.81 (m, 1H), 6.42-6.33 (m, 1H), 6.01 (dddd, J=1.8, 8.7, 17.3, 19.3 Hz, 1H), 4.58-4.38 (m, 2H), 3.94-3.81 (m, 1H), 3.80-3.70 (m, 7H), 3.68-3.53 (m, 2H), 2.81-2.71 (m, 1H), 2.71-2.61 (m, 2H), 2.54-2.39 (m, 1H), 2.23 (dtd, J=5.0, 6.8, 13.8 Hz, 1H), 1.93 (s, 3H), 1.22-1.15 (m, 12H);

³¹PNMR: (162 MHz, CDCl₃) δ=149.34 (s, 1P), 149.32 (s, 1P), 20.04 (s, 1P), 19.68 (s, 1P), 14.12 (s, 1P);

LCMS: (M−H⁺): 545.1, LCMS purity: 93.80%;

¹³CNMR: (101 MHz, CDCl₃) δ=163.84, 163.82, 150.58, 150.51, 148.58, 135.10, 135.02, 129.31, 118.68, 118.19, 117.80, 117.65, 116.79, 116.31, 111.73, 84.78, 84.74, 84.61, 84.53, 84.49, 84.39, 84.32, 75.66, 60.34, 58.05, 52.60, 52.54, 52.50, 52.47, 43.31, 38.42, 38.37, 24.59, 24.48, 24.45, 24.53, 20.45, 20.37, 20.36, 20.28, 14.16, 12.50, 12.48;

HPLC: HPLC purity: 95.15%;

TLC (Dichloromethane/Methanol) Rf=0.06.

### Example 21: Synthesis of 5′-(R)-Me-PO(OMe)2-Phosphonate-dT (WV-NU-128), and 5′-(R)-Me-PO(OMe)2-Phosphonate-3′-CNE-dT Phosphoramidite (WV-NU-128-CNE)

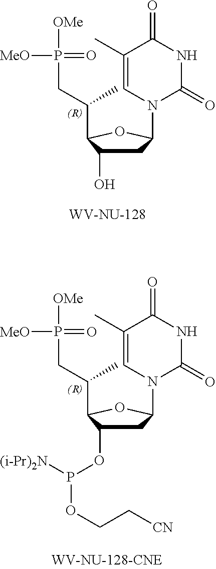

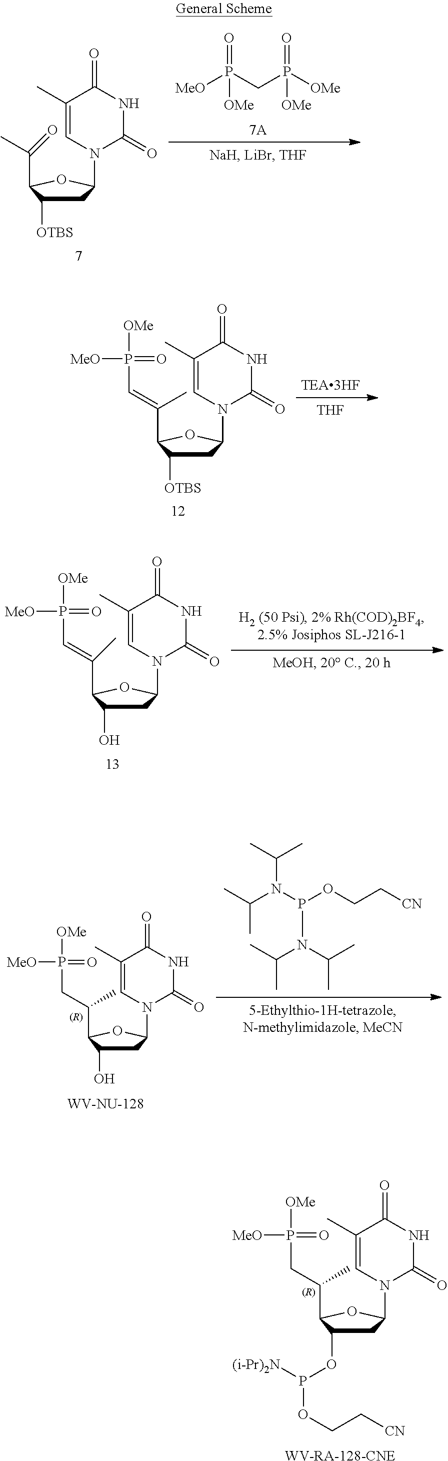

### 1. Preparation of Compound 12

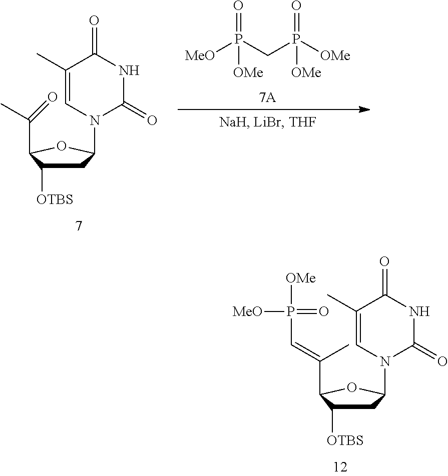

To a solution of NaH (4.78 g, 119.40 mmol, 60% purity) in THF (360 mL) was added bis(dimethoxyphosphoryl)methane (46.19 g, 119.40 mmol) in THF (200 mL) at 0° C. The reaction mixture was warmed up to 20° C., and stirred for 1 hr. A solution of LiBr (10.37 g, 119.40 mmol) in THF (200 mL) was added and the resultant slurry was stirred, and then cooled to 0° C. To the above mixture was added a solution of compound 7 (20 g, 54.27 mmol) in THF (200 mL) at 0° C. The mixture was stirred at 0-20° C. for 11 hr. TLC indicated compound 7 was consumed and two new spots formed. The resulting mixture was diluted with water (300 mL), extracted with EtOAc (300 mL*3). The combined organic layers were dried over anhydrous Na₂SO₄, filtered and concentrated to afford a yellow oil. The crude compound 12 (25 g, crude) was obtained as a yellow oil. The residue was purified by column chromatography (SiO₂, Petroleum ether/Ethyl acetate=1/10 to 0/1, then ethyl acetate/Methanol=10/1). Compound 12 (6.1 g, 24.40% yield) was obtained as a white solid.

LCMS: M+H⁺=475.2

TLC: (Ethyl acetate:Petroleum ether=3:1), R_{f}=0.20

### 2. Preparation of Compound 13

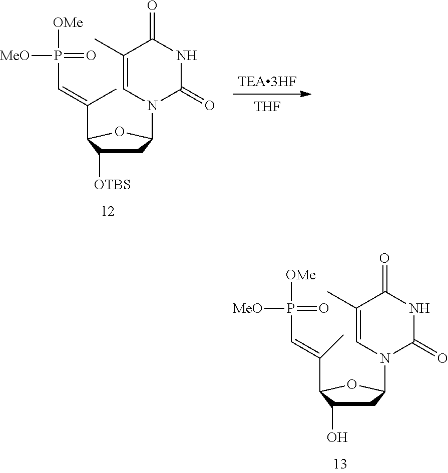

To a solution of compound 12 (6.1 g, 12.85 mmol) in THF (61 mL) was added 3HF·TEA (8.29 g, 51.42 mmol). The mixture was stirred at 25° C. for 12 hr. TLC indicated compound 12 was consumed and one new spot formed. The reaction mixture was quenched by addition sat. NaHCO₃ aq. (60 mL) and NaHCO₃ solid to pH=7˜8 and stirred 20 min. The mixture was dried over Na₂SO₄, and concentrated under reduced pressure to give a residue. The crude compound 13 (4.4 g, crude) was obtained as a yellow oil.

TLC: (Petroleum ether:Ethyl acetate=0:1) R_{f}=0

### 3. Preparation of Compound WV-NU-128

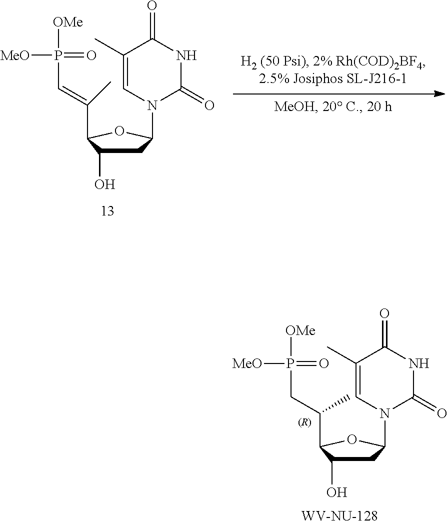

To a solution of compound 13 (5 g, 13.88 mmol) in MeOH (220 mL) were added Josiphos SL-J216-1 (425 mg, 1.39 mmol) (1Z,5Z)-cycloocta-1,5-diene; rhodium(1+); tetrafluoroborate (230 mg) and zinc; trifluoromethanesulfonate (2.06 g, 5.55 mmol). The mixture was stirred at 25° C. for 12 hr in H₂ (50 psi). LCMS showed the compound 13 was consumed and the main peak was desired. The reaction mixture was concentrated under reduced pressure to remove solvent. The residue was purified by column chromatography (SiO₂, Petroleum ether/Ethyl acetate=1/0 to 0/1 then ethyl acetate/methanol=10:1). The crude compound WV-NU-128 (4 g, 79.55% yield) was obtained as a yellow solid.

LCMS: M+H⁺=363.2

TLC: (Ethyl acetate:Methanol=10:1), R_{f}=0.36.

### 4. Preparation of Compound WV-RA-128-CNE

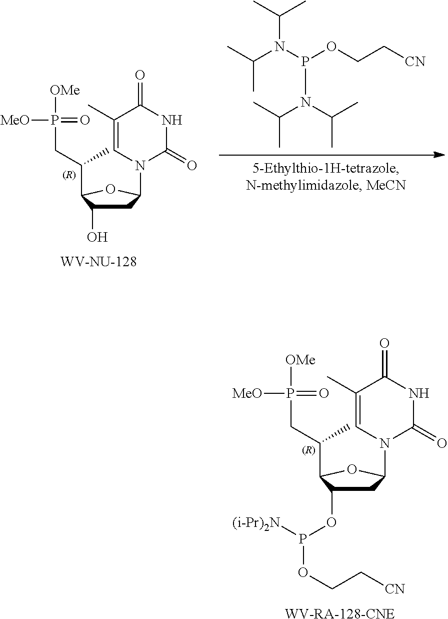

To a solution of compound WV-NU-128 (4 g, 11.04 mmol) in DMF (28 mL) was added 5-ethylsulfanyl-2H-tetrazole (1.44 g, 11.04 mmol) and 1-methylimidazole (1.81 g, 22.08 mmol), then added 3-bis(diisopropylamino)phosphanyloxypropanenitrile (4.99 g, 16.56 mmol). The mixture was stirred at 25° C. for 2 hr. TLC indicated compound WV-NU-128 was consumed and two new spots formed. The reaction mixture was added sat. NaHCO₃ aq. (50 mL) at 0° C., and then diluted with EtOAc (20 mL) and extracted with EtOAc (20 mL*3), dried over Na₂SO₄, filtered and concentrated under reduced pressure to give a residue. The residue was purified by column chromatography (SiO₂, Petroleum ether/Ethyl acetate=1/0 to 0/1, then Ethyl acetate/Acetonitrile=10/1, 5% TEA). Compound WV-RA-128-CNE (2.5 g, 40.25% yield) was obtained as a yellow oil.

¹H NMR (400 MHz, CDCl₃) δ=7.11 (d, J=1.0 Hz, 1H), 6.29-6.15 (m, 1H), 4.44-4.29 (m, 1H), 3.93-3.79 (m, 2H), 3.75 (dd, J=5.9, 10.8 Hz, 7H), 3.63 (br d, J=2.5 Hz, 2H), 2.74-2.60 (m, 2H), 2.53-2.36 (m, 1H), 2.34-2.21 (m, 1H), 2.19-2.06 (m, 2H), 1.93 (s, 3H), 1.78-1.61 (m, 1H), 1.21-1.09 (m, 15H)

¹³C NMR (101 MHz, CDCl₃) δ=163.54, 135.21, 135.05, 111.51, 83.59, 83.50, 77.36, 77.05, 76.72, 52.35, 52.32, 43.34 (dd, J=7.3, 12.5 Hz, 1C), 30.82, 29.10, 24.66, 24.63, 24.59, 24.50 (dd, J=2.9, 8.1 Hz, 1C), 16.21, 12.60

LCMS: M−H⁺=561.2, purity 93.7%

TLC: (Ethyl acetate:Methanol=8:1) R_{f}=0.45

### Example 22: Synthesis of 5′-Me-PO(OEt)2-Vinylphosphonate-dT (WV-NU-038)

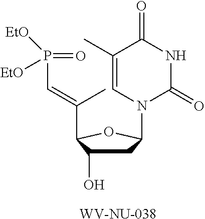

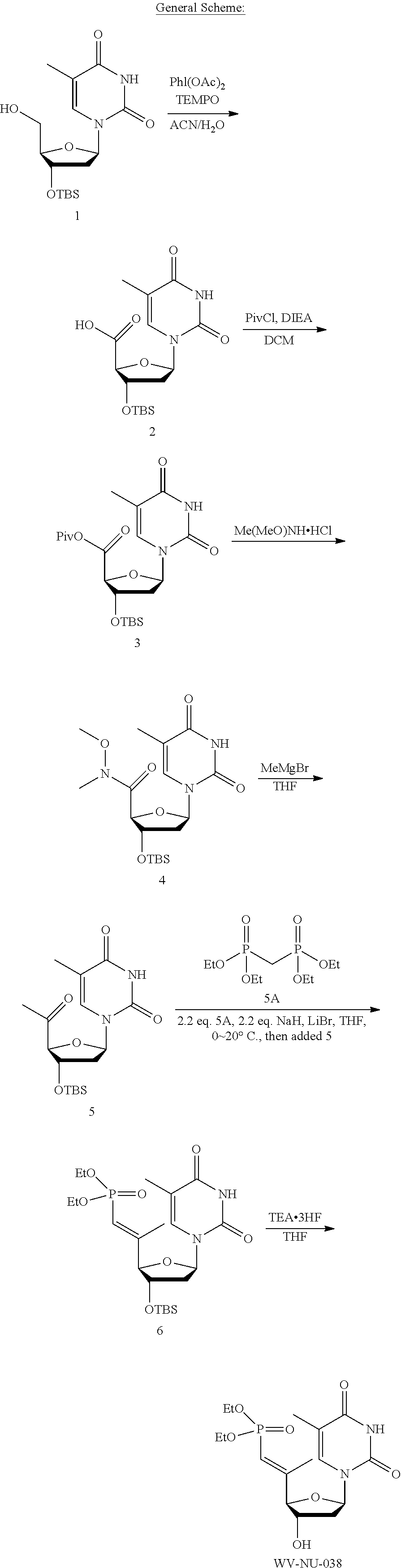

### 1. Preparation of Compound 2

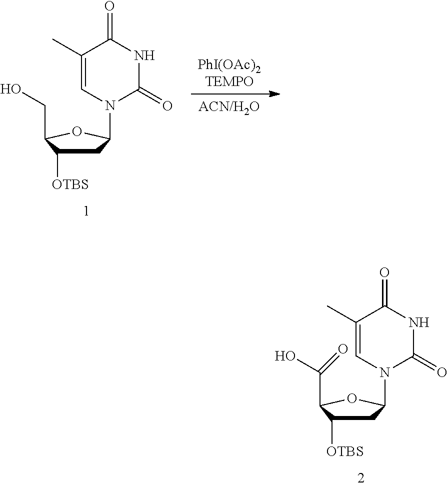

To a solution of compound 1 (15 g, 42.08 mmol) in the mixture of ACN (60 mL) and H₂O (60 mL) was added PhI(OAc)₂ (29.82 g, 92.57 mmol) and TEMPO (1.32 g, 8.42 mmol) at 20° C. The mixture was stirred at 20° C. for 2 hr. TLC showed the reaction was completed. The resulting mixture was concentrated to dry to give a residue, which was triturated with ACN (100 mL), filtered, and the filter cake was rinse with ACN (50 mL) and dried to give compound 2 (10.6 g, 68.00% yield) as a white solid. The combined filtrate was concentrated under reduced pressure and dried to give another part of crude product (7.4 g).

TLC (Ethyl acetate/Petroleum ether=1:1) Rf=0.01.

### 2. Preparation of Compound 3

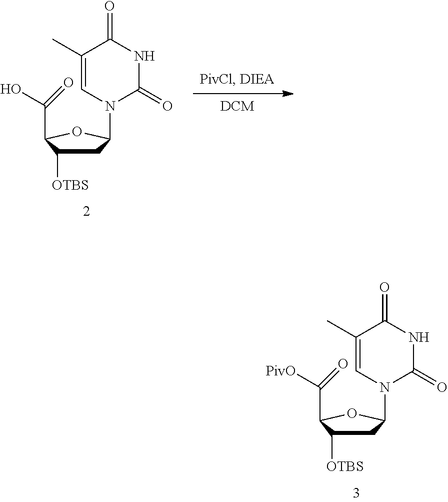

To a solution of crude compound 2 (7.4 g, 19.97 mmol) in DCM (70 mL) was added DIEA (5.16 g, 39.95 mmol, 6.96 mL) and pivaloyl chloride (3.13 g, 25.97 mmol). The mixture was stirred at −10-0° C. for 1.5 hr. TLC showed the reaction was almost completed. The crude brown color solution of compound 3 (9.08 g, 100.00% yield) in DCM was used directly for the next step.

TLC (Ethyl acetate/Petroleum ether=1:1) Rf=0.28.

### 3. Preparation of Compound 4

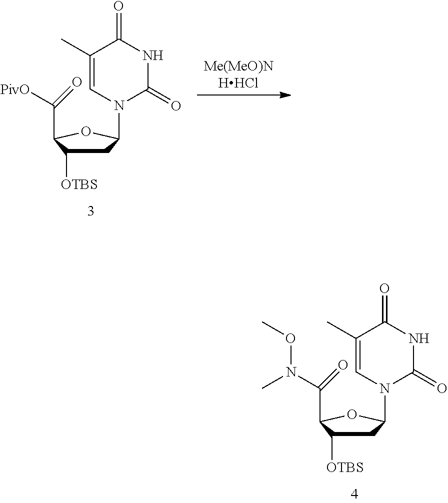

To the crude solution of compound 3 (9.08 g, 19.97 mmol) in DCM from the last step was added TEA (6.06 g, 59.92 mmol) followed by N-methoxymethanamine; hydrochloride (5.85 g, 59.92 mmol) at 0° C. The mixture was stirred at 0° C. for 1 hr. TLC showed the reaction was almost completed. The resulting mixture was washed with HCl (1N, 60 mL*2) and then aqueous NaHCO₃ (50 mL*2). The combined organic layers were dried over anhydrous Na₂SO₄, filtered and concentrated to get the product as a crude brown solid (10 g). The crude was purified by column chromatography on silica gel (Petroleum ether:Ethyl acetate, 10%˜60%). Compound 4 (2.7 g, 6.53 mmol, 32.69% yield) was obtained as a white solid.

TLC (Petroleum ether/Ethyl acetate=1:1) Rf=0.43.

### 4. Preparation of Compound 5

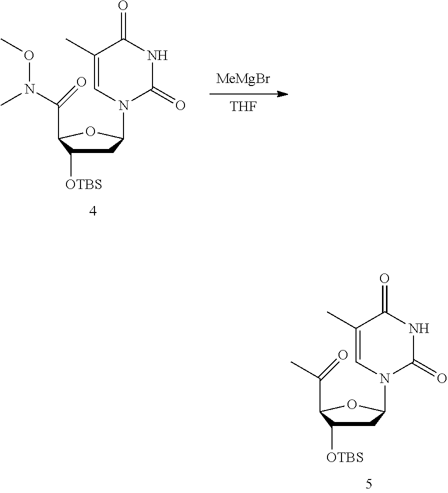

To a solution of compound 4 (17.2 g, 41.59 mmol) in THF (170 mL) was added MeMgBr (3 M, 27.73 mL) at 0° C. The mixture was stirred at 0° C. for 1.5 hr. TLC showed the reaction was completed. The resulting mixture was poured into sat. NH₄Cl aq. (300 mL) under stirring, extracted with EtOAc (100 mL*3). The combined organic layers were dried over anhydrous Na₂SO₄, filtered and concentrated to give a light yellow gum. The crude product was purified by column chromatography on silica gel (Petroleum ether:Ethyl acetate=8:1, 4:1, 2:1). Compound 5 (10.9 g, 67.14% yield, >94.4% purity) was obtained as a white solid.

HNMR (400 MHz, CDCl₃) Shift=8.76 (br s, 1H), 7.95 (s, 1H), 6.41 (dd, J=5.7, 7.9 Hz, 1H), 4.55-4.41 (m, 2H), 2.33-2.21 (m, 4H), 2.03-1.87 (m, 4H), 0.92 (s, 9H), 0.14 (d, J=3.5 Hz, 6H)

LCMS (M+H⁺) 369.3;

TLC (Petroleum ether/EtOAc=1:1, two times) Rf=0.63.

### 5. Preparation of Compound 6

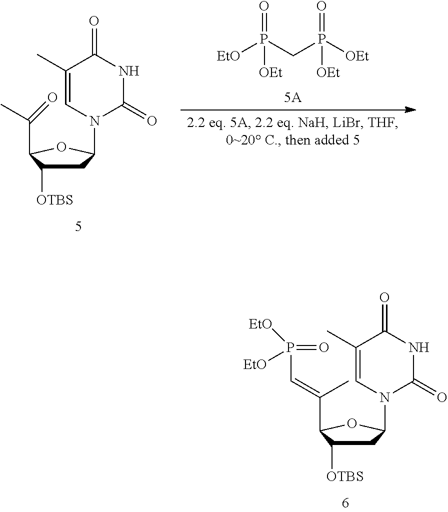

To a suspension of NaH (4.78 g, 119.40 mmol, 60% purity) in THF (100 mL) was added compound 5A (34.41 g, 119.40 mmol) in THF (100 mL) at 0° C. The reaction mixture was warmed up to 20° C., and stirred for 1 hr. A solution of LiBr (10.37 g, 119.40 mmol) in THF (100 mL) was added, and the resultant slurry was stirred, and then cooled to 0° C. To the above mixture was added a solution of compound 5 (20 g crude, 54.27 mmol) in THF (100 mL) at 0° C. The reaction mixture was stirred at 0° C. for 1 hr and then allowed to warm up to 20° C., and stirred at 20° C. for 64 hr. TLC showed compound 5 was remained and one main spot was detected. The resulting mixture was diluted with water (400 mL), extracted with EtOAc (400 mL*3). The combined organic layers were dried over anhydrous Na₂SO₄, filtered and concentrated to afford a yellow oil. The residue was purified by flash silica gel chromatography (ISCO@; 220 g SepaFlash@Silica Flash Column, Eluent of 0˜50% Ethyl acetate/Petroleum ethergradient @100 mL/min). Compound 6 (6.7 g, 21.69% yield, 88.3% purity) was obtained as a yellow oil.

LCMS: (M+H⁺):503.1;

TLC (Petroleum ether/Ethyl acetate=1:3) Rf=0.11.

### 6. Preparation of Compound WV-NU-038

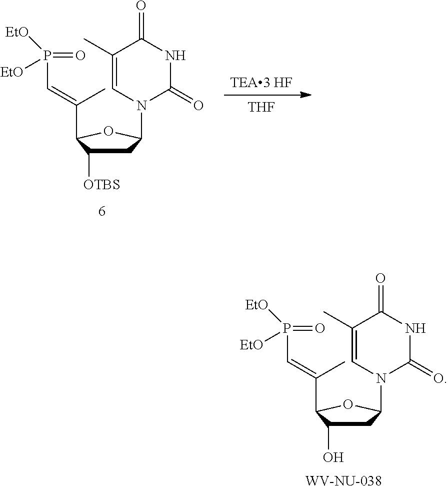

To a solution of compound 6 (6.4 g, 12.73 mmol) in THF (64 mL) was added TEA·3HF (8.38 g, 50.93 mmol). The mixture was stirred at 15° C. for 12 hr. LCMS showed compound 6 was remained and one main peak with desired mass was detected. The reaction mixture was quenched by addition NaHCO₃ (aq., sat. 64 mL), and then extracted with Ethyl acetate (70 mL*3). The combined organic layers were dried over Na₂SO₄, filtered and concentrated under reduced pressure to give a residue. The residues was purified by flash silica gel chromatography (ISCO®; 80 g SepaFlash® Silica Flash Column, Eluent of 0˜60% Ethyl acetate/Petroleum ethergradient @100 mL/min). The compound WV—NU-038 (2.85 g, 56.35% yield, 97.78% purity) was yellow solid.

1H NMR (400 MHz, CHLOROFORM-d) 6=9.61 (s, 1H), 7.14 (s, 1H), 6.36 (t, J=6.8 Hz, 1H), 5.78 (d, J=17.9 Hz, 1H), 4.56 (br s, 1H), 4.36 (br s, 1H), 4.26 (br d, J=4.0 Hz, 1H), 4.13-3.96 (m, 4H), 2.36 (ddd, J=4.0, 6.3, 13.7 Hz, 1H), 2.25-2.12 (m, 4H), 1.92 (s, 3H), 1.38-1.28 (m, 7H)).

13C NMR (101 MHz CDCl₃,) δ=163.77, 158.00, 157.92, 150.70, 135.49, 112.27, 111.75, 88.91, 88.69, 84.46, 73.57, 61.81, 61.65, 39.18, 16.61, 16.54, 16.41, 16.35, 16.30, 12.63).

31P NMR (162 MHz, CDCl₃) δ=17.63 (s, 1P).

LCMS: (M+H)=389.1; LCMS purity: 99.2%.

### Example 23: Synthesis of 5′-(R)-Me-PO(OEt)₂-dT (WV-NU-037) and 5′-(S)-Me-PO(OEt)₂-dT (WV-NU-037A)

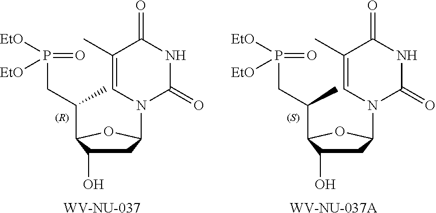

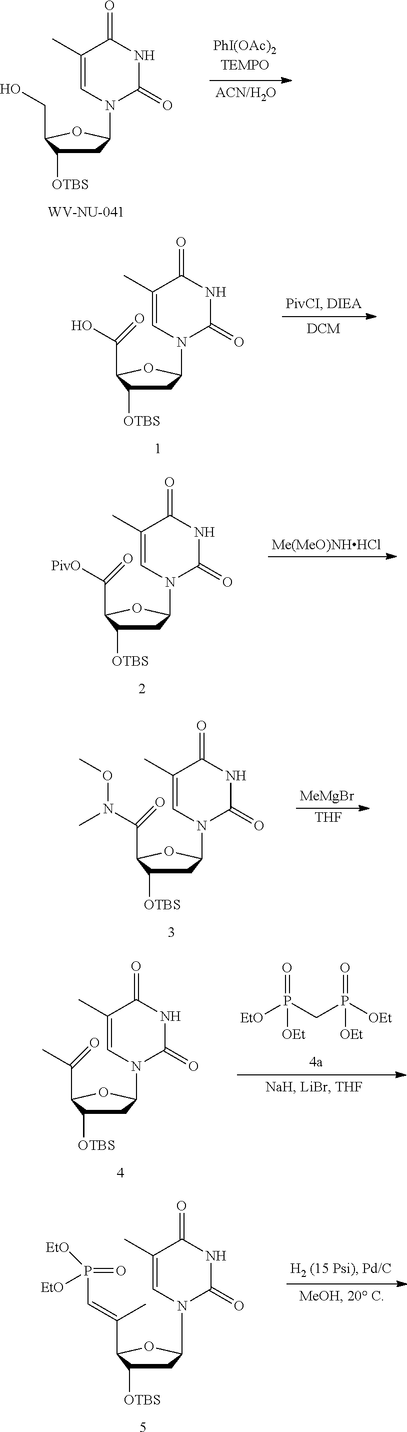

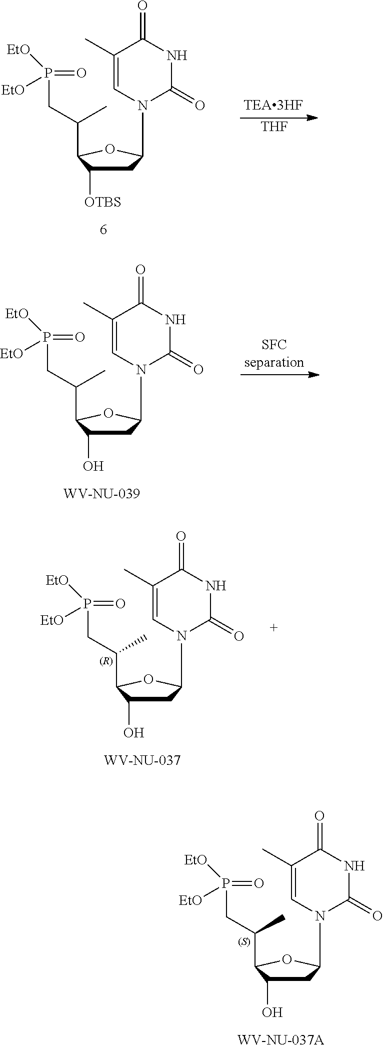

### 1. Preparation of Compound 1

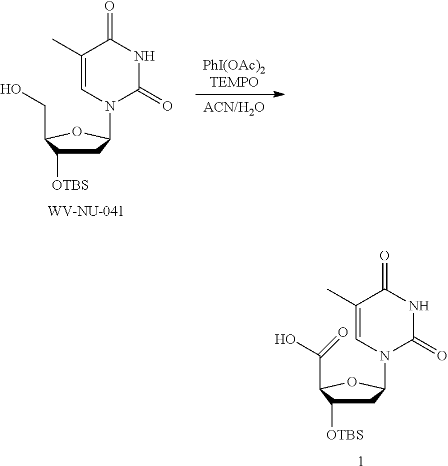

To a solution of compound WV-NU-041 (150 g, 420.77 mmol) in the mixture of ACN (600 mL) and H₂O (600 mL) was added PhI(OAc)₂ (298.17 g, 925.70 mmol) and TEMPO (13.23 g, 84.15 mmol). The mixture was stirred at 20° C. for 2 hr. TLC showed the reaction was completed. The resulting mixture was concentrated to dry to give a residue, which was triturated with ACN (550 mL), filtered, and the filter cake was rinsed with ACN (300 mL) and dried to give compound 1 (113.4 g, 72.75% yield) as a white solid.

¹H NMR (400 MHz, METHANOL-d₄) δ=8.28 (s, 1H), 6.44 (dd, J=5.0, 9.0 Hz, 1H), 4.69 (d, J=4.4 Hz, 1H), 4.42 (s, 1H), 2.25 (dd, J=5.0, 13.4 Hz, 1H), 2.09-1.97 (m, 1H), 1.89 (s, 3H), 0.94 (s, 9H), 0.17 (d, J=4.8 Hz, 6H)

LCMS: (M+H⁺): 371.2

TLC (Petroleum ether:Ethyl acetate=1:1), Rf=0.00

### 2. Preparation of Compound 2

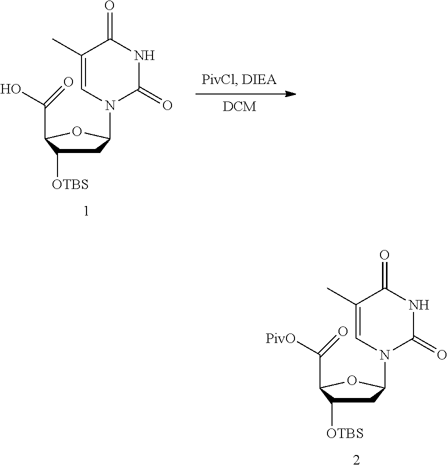

To a solution of compound 1 (110 g, 296.92 mmol) in DCM (1100 mL) was added DIEA (76.75 g, 593.84 mmol) and 2,2-dimethylpropanoyl chloride (46.54 g, 385.99 mmol). The mixture was stirred at −10-0° C. for 1.5 hr. TLC indicated compound 1 was remained a little and two new spots formed. The crude product compound 2 (134.98 g, 100.00% yield) in DCM was used into the next step without further purification.

### 3. Preparation of Compound 3

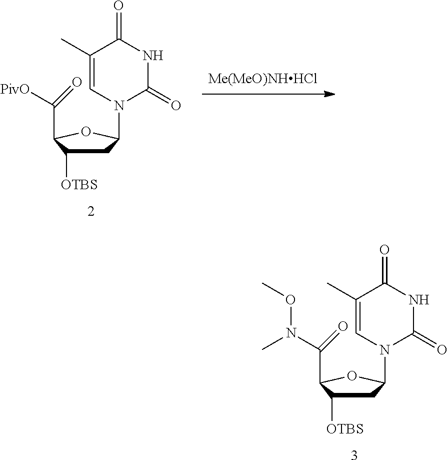

To a solution of compound 2 (134.9 g, 296.75 mmol) in DCM was added TEA (90.09 g, 890.26 mmol), then added N-methoxymethanamine; hydrochloride (86.84 g, 890.26 mmol). The mixture was stirred at 0° C. for 2 hr. TLC indicated compound 2 was consumed and one new spot formed. The resulting mixture was washed with HCl (1N, 800 mL*2) and then aqueous NaHCO₃ (600 mL*2). The combined organic layers were dried over anhydrous Na₂SO₄, filtered and concentrated to get the product as a crude white solid. The residue was purified by column chromatography (SiO₂, Petroleum ether/Ethyl acetate=I/O to 0:1). Compound 3 (115 g, 93.71% yield) was obtained as a white solid.

¹H NMR (400 MHz, CDCl₃) δ=8.62 (br s, 1H), 8.38 (s, 1H), 6.57 (dd, J=5.1, 9.3 Hz, 1H), 4.81 (s, 1H), 4.48 (br d, J=4.0 Hz, 1H), 3.80-3.72 (m, 3H), 3.25 (s, 3H), 2.23 (dd, J=5.1, 13.0 Hz, 1H), 2.09-2.01 (m, 1H), 1.97 (d, J=1.0 Hz, 3H), 0.91 (s, 9H), 0.10 (d, J=3.8 Hz, 6H)

LCMS: (M+H⁺): 414.2

TLC (Petroleum ether:Ethyl acetate=1:1), R_{f}=0.39

### 4. Preparation of Compound 4

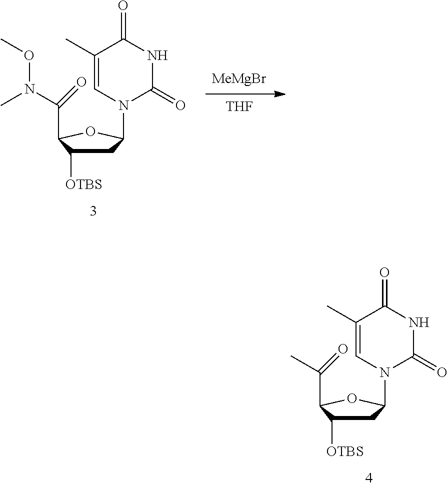

To a solution of compound 3 (115 g, 278.09 mmol) in THF (1150 mL) was added MeMgBr (3 M, 185.39 mL). The mixture was stirred at 0° C. for 1.5 hr. TLC indicated compound 3 was consumed and two new spots formed. The resulting mixture was poured into sat. NH₄Cl aq. (1000 mL) under stirring, extracted with EtOAc (500 mL*3). The combined organic layers were dried over anhydrous Na₂SO₄, filtered and concentrated to give a light yellow gum. The residue was purified together with another batch crude (14.5 g-scale of Cpd.3) by column chromatography (SiO₂, Petroleum ether/Ethyl acetate=1/0 to 0:1). Compound 4 (93.2 g, 90.95% yield) was obtained as a white solid.

¹H NMR (400 MHz,) CDCl₃ 6=7.96 (s, 1H), 6.41 (dd, J=5.5, 8.2 Hz, 1H), 4.52 (d, J=2.2 Hz, 1H), 4.47 (td, J=2.2, 4.9 Hz, 1H), 2.30-2.23 (m, 4H), 2.00-1.92 (m, 4H), 0.92 (s, 9H), 0.13 (d, J=3.5 Hz, 6H)

LCMS: (M+H⁺): 369.2

TLC (Petroleum ether:Ethyl acetate=1:1), R_{f}=0.61

### 5. Preparation of Compound 5

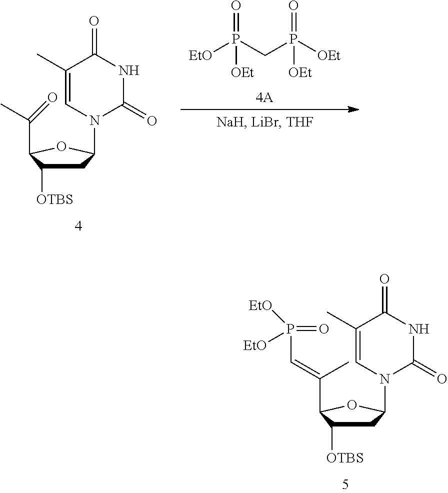

To a solution of NaH (21.49 g, 537.31 mmol, 60% purity) in THF (594 mL) was added 1-[diethoxyphosphorylmethyl(ethoxy)phosphoryl]oxyethane (154.86 g, 537.31 mmol) in THF (594 mL) at 0° C. The reaction mixture was warmed up to 20° C., and stirred for 1 hr. A solution of LiBr (46.66 g, 537.31 mmol) in THF (594 mL) was added and the resultant slurry was stirred, and then cooled to 0° C. To the above mixture was added a solution of compound 4 in THF (468 mL) at 0° C. The mixture was stirred at 0-20° C. for 71 hr. TLC indicated compound 4 was remained a little and three new spots formed. The resulting mixture was diluted with water (100 mL), extracted with EtOAc (100 mL*3). The combined organic layers were dried over anhydrous Na₂SO₄, filtered and concentrated to afford a yellow oil. The residue was purified by column chromatography (SiO₂, Petroleum ether/Ethyl acetate=1/0 to 0:1). Compound 5 (48.74 g, 9.41% yield) was obtained as a yellow oil.

LCMS: (M+H⁺):503.1

TLC (Ethyl acetate:Petroleum ether=3; 1), R_{f}=0.28

### 6. Preparation of Compound 6

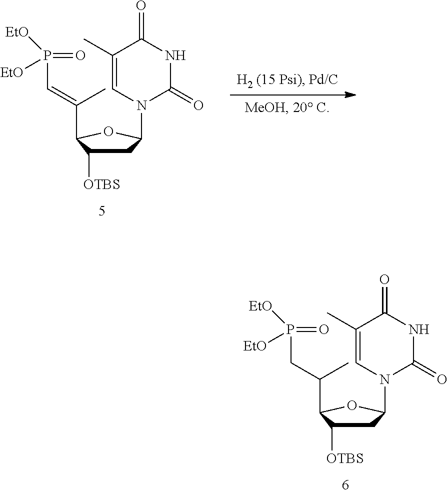

To a solution of compound 5 (40 g, 79.58 mmol) in MeOH (100 mL) was added Pd/C (8 g, 10% purity) (50% water) and H₂ (15 psi). The mixture was stirred at 20° C. for 2 hr. LCMS showed the compound 5 was consumed and the main peak was desired. Filtered the Pd/C then concentrated under reduced pressure to give a residue. Compound 6 (40.16 g, crude) was obtained as a yellow oil.

LCMS: (M+H⁺):505.2, 505.1

### 7. Preparation of Compound WV-NU-039

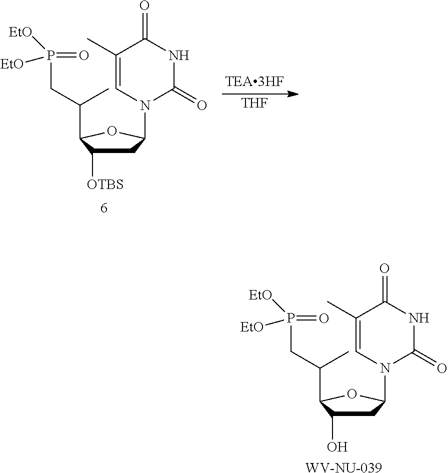

To a solution of compound 6 (40.16 g, 79.58 mmol) in THF (400 mL) was added N,N-diethylethanamine; trihydrofluoride (51.32 g, 318.33 mmol). The mixture was stirred at 25° C. for 16 hr. LCMS showed the compound 6 was consumed and the main peak was desired. The reaction mixture was quenched by addition Na₂CO₃ (aq. sat. 400 mL), and extracted with Ethyl acetate (400 mL*3). The combined organic layers were dried over Na₂SO₄, filtered and concentrated under reduced pressure to give a residue. The residue was purified by column chromatography (SiO₂, Petroleum ether/Ethyl acetate=1/0 to 0:1). Compound WV-NU-039 (30 g, 96.57% yield) was obtained as a yellow oil. Then purified by SFC (column: DAICEL CHIRALPAK ADH (250 mm*30 mm, 5 um); mobile phase: [Neu-ETOH]; B %: 35%-35%, 8.5 min).

LCMS: (M+H⁺):390.9

### 8. Preparation of Compound NU-037, WV-NU-037A

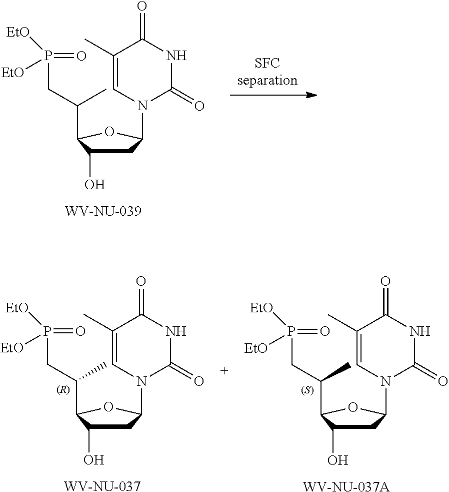

Compound WV-NU-037 (11.3 g, 37.43% yield, 99.37% purity) was obtained as a white solid.

¹H NMR (400 MHz CDCl₃,) δ=9.49-9.10 (m, 1H), 7.11 (d, J=1.1 Hz, 1H), 6.22 (t, J=6.6 Hz, 1H), 4.54 (br d, J=4.9 Hz, 1H), 4.28 (br dd, J=4.0, 7.3 Hz, 1H), 4.20-4.00 (m, 4H), 3.68 (dd, J=5.1, 7.3 Hz, 1H), 2.38 (ddd, J=4.5, 6.6, 13.8 Hz, 1H), 2.29-1.97 (m, 3H), 1.93 (s, 3H), 1.83-1.63 (m, 1H), 1.34 (dt, J=3.7, 7.1 Hz, 6H), 1.17 (d, J=6.6 Hz, 3H).

¹³C NMR (101 MHz, CDCl₃ 6=163.91, 150.51, 135.20, 111.30, 89.49, 89.36, 83.53, 72.16, 61.94 (dd, J=6.6, 13.2 Hz, 1C), 58.29, 40.29, 31.55, 31.52, 29.95, 28.56, 18.38, 17.77, 17.69, 16.47, 16.40, 12.71

LCMS: (M+H⁺): 391.1; LCMS purity: 99.37%

SFC: (AD-3_EtOH_IPAm_10-40_Gradient 4 ml), SFC purity=100.00%;

Compound WV-NU-037A (16.2 g, 54.00% yield, 100% purity) was obtained as a white solid.

¹HNMR (400 MHz, CDCl₃) δ=8.93 (s, 1H), 7.19 (d, J=1.1 Hz, 1H), 6.29 (dd, J=6.3, 7.6 Hz, 1H), 4.35 (qd, J=3.5, 7.1 Hz, 1H), 4.22-4.02 (m, 4H), 3.88-3.75 (m, 2H), 2.37 (ddd, J=3.2, 6.1, 13.8 Hz, 1H), 2.31-2.14 (m, 1H), 2.13-2.02 (m, 2H), 1.95 (d, J=0.9 Hz, 3H), 1.69 (ddd, J=8.2, 15.4, 18.7 Hz, 1H), 1.34 (dt, J=5.5, 6.9 Hz, 6H), 1.17 (d, J=6.6 Hz, 3H).

¹³CNMR (101 MHz, CDCl₃) δ=163.88, 150.59, 135.33, 111.59, 89.39, 89.26, 83.94, 71.49, 61.84 (dd, J=6.2, 20.2 Hz, 1C), 40.02, 31.41, 31.38, 29.15, 27.75, 17.12, 17.06, 16.45 (dd, J=2.2, 5.9 Hz, 1C), 12.53

LCMS: (M+H⁺): 391.1; LCMS purity: 100%

SFC: (AD-3_EtOH_IPAm_10-40_Gradient_4 ml), SFC purity=100.00%.

### Example 24: Modified Method of Synthesis of 5′-(R)-Me-PO(OEt)₂-phosphonate-dT (WV-NU-037)

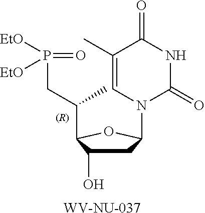

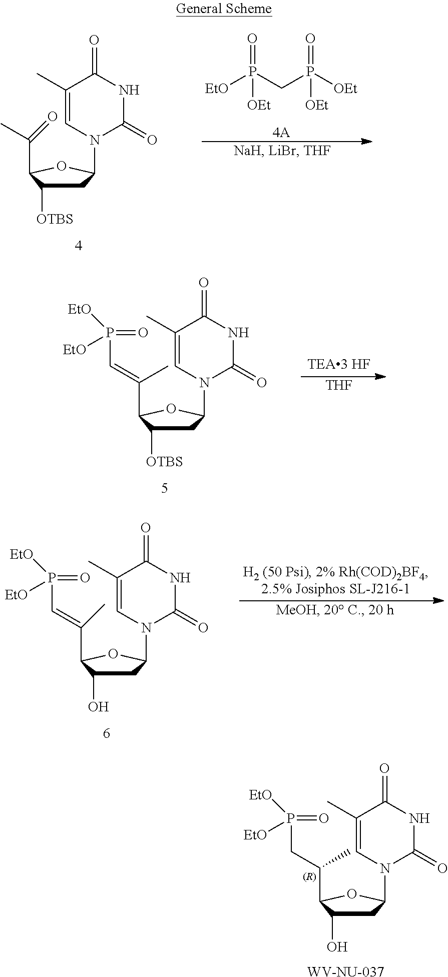

### 1. Preparation of Compound 5

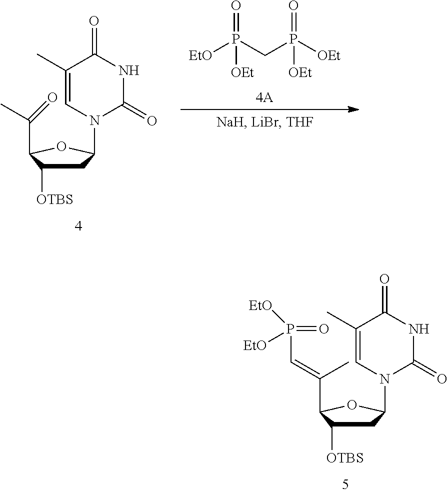

To a solution of NaH (23.88 g, 597.02 mmol, 60% purity) in THF (900 mL) was added compound 4A (172.07 g, 597.02 mmol) in THF (500 mL) at 0° C. The reaction mixture was warmed up to 20-30° C., and stirred for 1 hr. A solution of LiBr (51.85 g, 597.02 mmol) in THF (500 mL) was added and the resultant slurry was stirred, and then cooled to 0° C. To the above mixture was added a solution of compound 4 (50 g, 135.69 mmol) in THF (500 mL) at 0° C. The mixture was stirred at 0-15° C. for 11 hr. TLC indicated compound 4 was consumed completely and desired spot formed. The resulting mixture was diluted with water (500 mL), extracted with EtOAc (500 mL*3). The combined organic layers were dried over anhydrous Na₂SO₄, filtered and concentrated to afford a yellow oil. The residue was purified by column chromatography (SiO₂, Petroleum ether/Ethyl acetate=5/1 to 1:1). Compound 5 (63 g, 90.44% yield, 97.9% purity) was obtained as a yellow solid.

¹H NMR (400 MHz, CDCl₃) δ=8.98 (br s, 1H), 7.05 (s, 1H), 6.24 (t, J=6.8 Hz, 1H), 5.71 (d, J=17.2 Hz, 1H), 4.23-3.97 (m, 8H), 2.25-2.12 (m, 1H), 2.11-2.03 (m, 4H), 1.88 (s, 3H), 1.30-1.07 (m, 9H), 0.83 (s, 9H), 0.04-0.00 (m, 7H).

LCMS: (M+H⁺): 503.1.

TLC (Ethyl acetate:Petroleum ether=3:1), R_{f}=0.16.

### 2. Preparation of Compound 6

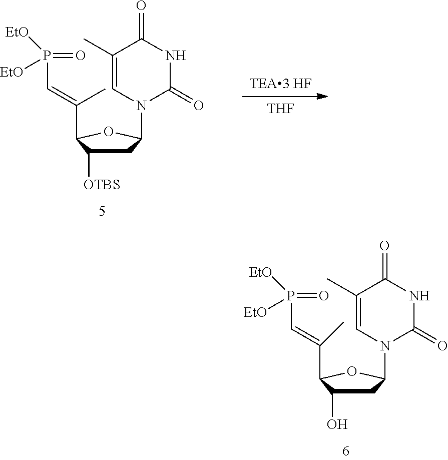

To a solution of compound 5 (57 g, 113.41 mmol) in THF (600 mL) was added N,N-diethylethanamine; trihydrofluoride (73.13 g, 453.63 mmol). The mixture was stirred at 15° C. for 6 hr. TLC showed compound 5 was consumed completely. One new spot was desired compound. The reaction mixture was quenched by addition sat. NaHCO₃ aq. (20 mL) and NaHCO₃ solid to pH=7˜8 and stirred 20 min. The mixture was dried over Na₂SO₄, and concentrated under reduced pressure to give a residue. Compound 6 (44 g, crude) was obtained as a yellow solid.

TLC (Ethyl acetate:Methanol=15:1), R_{f}=0.43.

### 3. Preparation of Compound WV-NU-037

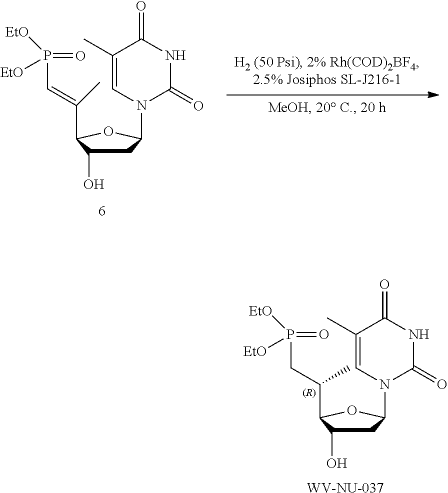

To a mixture of compound 6 (43 g, 110.72 mmol) in MeOH (1840 mL) was added (1Z,5Z)-cycloocta-1,5-diene; rhodium(1+); tetrafluoroborate (1.80 g, 4.43 mmol), Josiphos SL-J216-1 (CAS #: 849924-43-2, 3.32 g, 5.09 mmol) and zinc; trifluoromethanesulfonate (16.10 g, 44.29 mmol). And the system was stirred under H₂ (50 psi) for 12 hr at 25° C. LC-MS showed compound 6 was consumed completely and one main peak with desired MS was detected. The reaction mixture was filtered and concentrated under reduced pressure to give a residue. Combined with 6 g crude. The residue was purified by flash silica gel chromatography (ISCO®; 80 g SepaFlash® Silica Flash Column, Eluent of 0˜100% Ethyl acetate/Petroleum ether gradient and 10% Petroleum ether gradient/MeOH @100 mL/min) to give 36.2 g (average weight 30.4 g). And 18.5 g crude. Compound WV-NU-037 (30.4 g, 77.88 mmol, 70.33% yield) was obtained as a black brown solid. And 18.5 g crude. The residue was purified by flash silica gel chromatography (ISCO®; 220 g SepaFlash® Silica Flash Column, Eluent of 20:60:20, 100:0; 20, Ethyl acetate/Petroleum/DCM ether gradient @80 mL/min). Then washed with 200 mL (Petroleum ether:Ethyl acetate=1:1) to give 39.8 g off white solid.

¹HNMR (400 MHz, CDCl₃) δ=8.68 (br s, 1H), 7.10 (s, 1H), 6.18 (t, J=6.5 Hz, 1H), 4.29 (br d, J=4.6 Hz, 2H), 4.20-4.04 (m, 4H), 3.66 (br dd, J=5.1, 7.3 Hz, 1H), 2.38 (td, J=6.7, 11.7 Hz, 1H), 2.26-1.97 (m, 4H), 1.93 (s, 4H), 1.84-1.70 (m, 1H), 1.34 (dt, J=4.5, 7.0 Hz, 7H), 1.17 (d, J=6.8 Hz, 3H)

³¹PNMR (162 MHz, CDCl₃) δ=31.55 (s, 1P), 31.49 (s, 1P)

¹³CNMR (101 MHz, CDCl₃) δ 163.52, 150.30, 135.35, 111.33, 89.42, 89.31, 83.74, 72.36, 62.15 (dd, J=6.6, 12.5 Hz, IC), 40.18, 31.70, 29.92, 28.52, 18.34, 18.26, 16.43, 16.36, 12.63

SFC: method (AD-3_EtOH_IPAm_10-40_Gradient_4 ml_A) dr=97.43:2.57

LCMS (M+H⁺): 391.1; LCMS purity: 99.37%

### Example 25: Synthesis of 5′-PO(OEt)₂-Triazolyl phosphonate-dT (WV-NU-040)

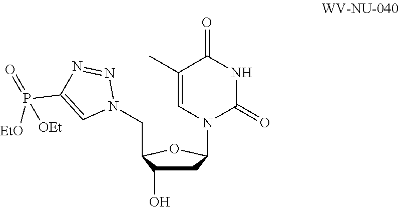

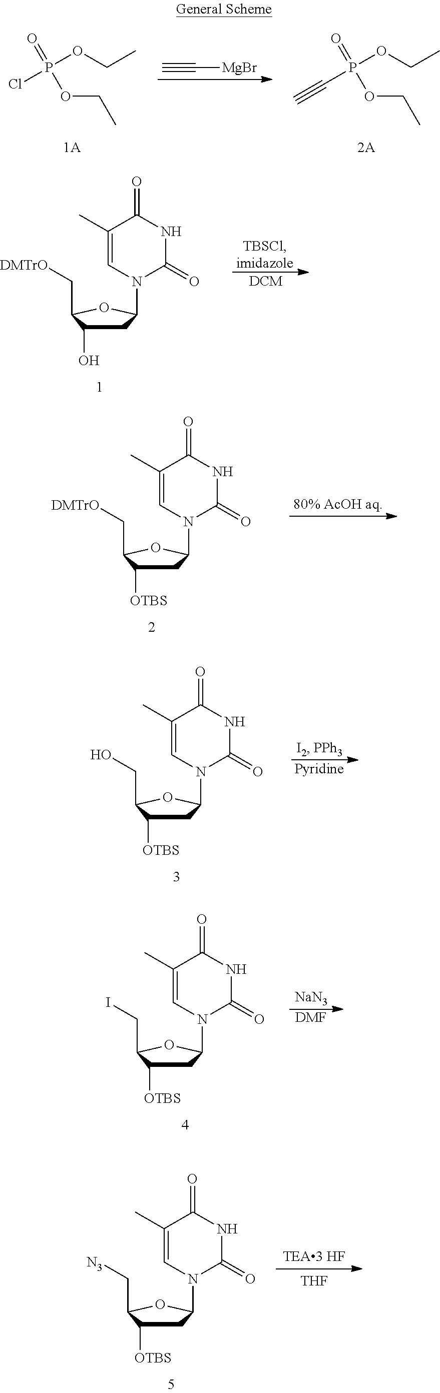

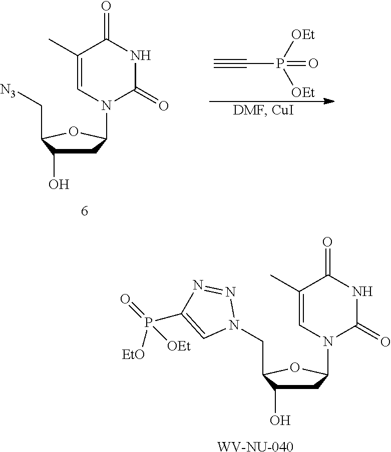

### 1. Preparation of Compound 2A

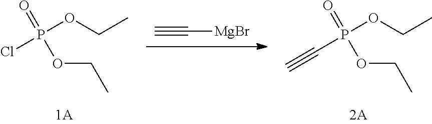

To a solution of compound 1A (10 g, 57.96 mmol) in THF (20 mL) was added to bromo(ethynyl)magnesium (0.5 M, 117.07 mL) at 0° C. under N₂. The resulting mixture was stirred at 20° C. for 0.5 hr. TLC showed compound 1A was consumed completely and two new spots formed. The mixture was quenched by addition sat. NH₄Cl (aq., 50 mL) at 0° C., then diluted with Ethyl acetate (30 mL) and extracted with Ethyl acetate (150 mL*3). The combined organic layers were dried over Na₂SO₄, filtered and concentrated under reduced pressure to give crude. The residue was purified by column chromatography (SiO₂, Petroleum ether/Ethyl acetate=10:1 to 1:1). Compound 2A (5.2 g, 55.34% yield) was obtained as a colorless oil.

LCMS: (M+H⁺): 163.3.

TLC (Petroleum ether/Ethyl acetate=1:1) R_{f}=0.43

### 2. Preparation of Compound 4

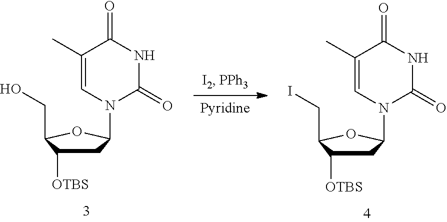

To a solution of compound 3 (10 g, 28.05 mmol) in pyridine (200 mL) was added PPh₃ (13.24 g, 50.49 mmol) and 12 (10.68 g, 42.08 mmol). The mixture was stirred at 25° C. for 12 hr under N₂ atmosphere. LCMS showed most of the starting mateiral was disappeared and one main peak with desired mass was detected. The reaction mixture was quenched by sat. aq. Na₂SO₃ (200 mL) and extracted with EtOAc (600 mL*3). The combined organic layers were washed with brine (200 mL*2), dried over Na₂SO₄, filtered and concentrated under reduced pressure to give a residue. The residue was purified by column chromatography (SiO₂, Petroleum ether/Ethyl acetate=10:1 to 0:1). Compound 4 (4.8 g, 33.40% yield, 91.041% purity) was obtained as a colorless oil.

LCMS: (M+H⁺): 467.0;

TLC (Petroleum ether/Ethyl acetate=1:3) R_{f}=0.75.

### 3. Preparation of Compound 5

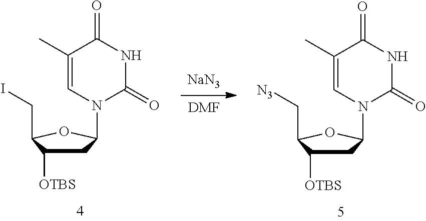

To a solution of compound 4 (4.8 g, 10.29 mmol) in DMF (48 mL) was added NaN₃ (802.89 mg, 12.35 mmol). The mixture was stirred at 50° C. for 12 hr. LCMS showed compound 4 was consumed completely and one main peak with desired MS was detected. The reaction was quenched by H₂O (6 mL), and extracted with TBME (6 mL*3). Compound 5 (3.93 g, crude) in a yellow solution of TBME (18 mL) was used into the next step without further purification.

LCMS: (M+H⁺): 382.3

### 4. Preparation of Compound 6

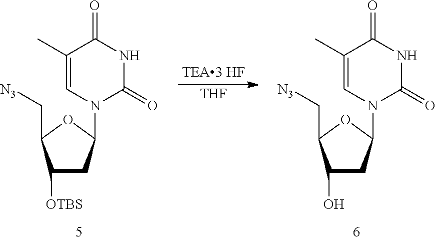 [SMILES: Cc1cn([C@H]2C[C@@H](O[Si](C)(C)C(C)(C)C)[C@@H](CN=[N+]=[N-])O2)c(=O)[nH]c1=O ; Cc1cn([C@H]2C[C@@H](O)[C@@H](CN=[N+]=[N-])O2)c(=O)[nH]c1=O ; "THF"]

To a solution of compound 5 (3.93 g, 10.30 mmol) in THF (20 mL) was added N,N-diethylethanamine; trihydrofluoride (6.64 g, 41.21 mmol). The mixture was stirred at 20° C. for 12 hr. TLC showed a few of compound 5 was remained and new spot was detected. The reaction mixture was concentrated under reduced pressure and the mixture was neutralized with Na₂CO₃ (aq., sat.) until pH=7. The mixture was concentrated under reduced pressure to removed most of water. The mixture was added DCM (40 mL) and dried over Na₂SO₄, filtered and concentrated under reduced pressure to give a residue. The residue was purified by prep-TLC (SiO₂, Petroleum ether: (Ethyl acetate: Ethyl Alcohol=3:1)=1:1). Compound 6 (2.7 g, crude) was obtained as a yellow oil.

TLC (Petroleum ether: (Ethyl acetate: Ethyl Alcohol=3:1)=1:1) R_{f}=0.24

### 5. Preparation of WV-NU-040

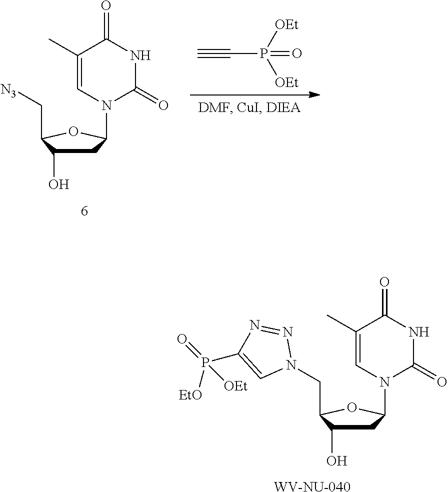

To a solution of compound 6 (2 g, 7.48 mmol) and 1-[ethoxy(ethynyl)phosphoryl]oxyethane (1.42 g, 8.76 mmol) in DMF (20 mL) was degassed and purged with N₂ for 3 times, then DIEA (1.93 g, 14.97 mmol), CuI (285.06 mg, 1.50 mmol) was added. The mixture was stirred at 20° C. for 4 hr under N₂ atmosphere. LCMS showed most of the starting material was disappeared and the desired substance was found. The reaction mixture was diluted with TMT solution (8 mL), filtered and the filtrate was diluted with ACN (80 mL), and concentrated under reduced pressure to give a residue. The residue was washed with EtOAc (100 mL*3), filtered and concentrated under reduced pressure to give product. WV—NU-040 (1.8 g, 3.92 mmol, 52.38% yield, 93.513% purity) was obtained as a white solid.

¹H NMR (400 MHz, DEUTERIUM OXIDE) 6 ppm 8.39 (s, 1H), 6.96 (s, 1H), 6.07 (t, J=6.4 Hz, 1H), 4.77 (d, J=4.4 Hz, 2H), 4.37 (q, J=6.2 Hz, 1H), 4.19 (q, J=4.9 Hz, 1H), 4.01-4.14 (m, 4H), 2.20-2.37 (m, 2H), 1.73 (s, 3H), 1.19 (s, 6H)

³¹P NMR (162 MHz, DEUTERIUM OXIDE) δ ppm 8.67 (s, 1 P)

¹³C NMR (101 MHz, DEUTERIUM OXIDE) δ=166.21, 151.53, 137.29, 136.50, 134.08, 133.47, 133.14, 111.55, 85.38, 82.53, 70.15, 64.72, 64.66, 50.60, 36.90, 15.47, 15.41, 11.49.

LCMS: (M+H⁺): 430.1, LCMS purity: 93.513%.

### Example 26: Synthesis of 5′-(POM)₂-Vinyl Phosphonate-dT (WV-NU-042)

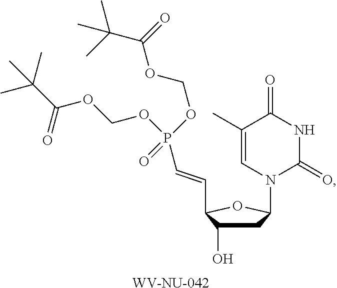

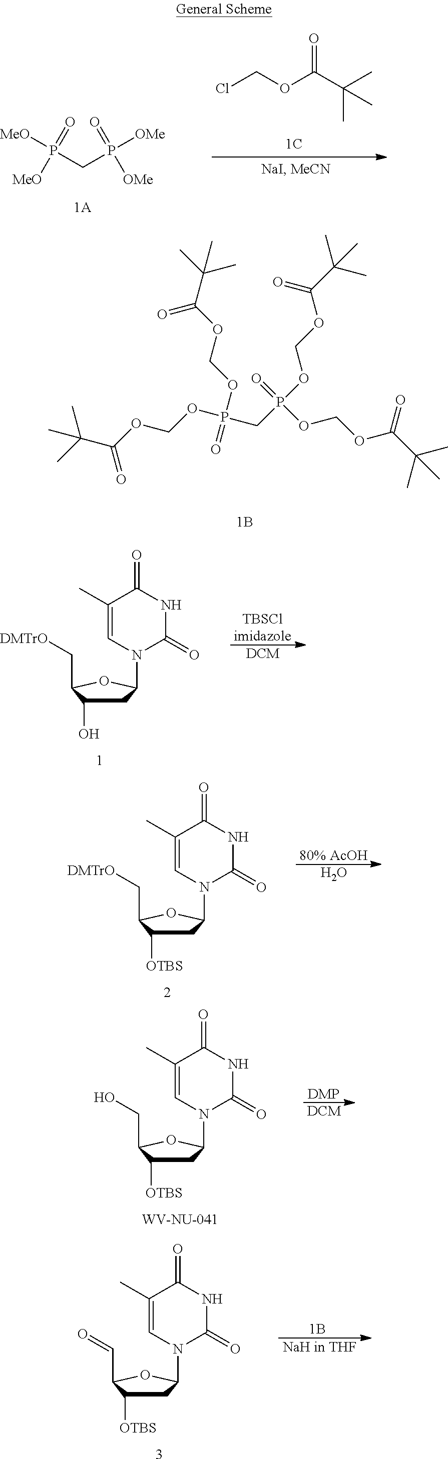

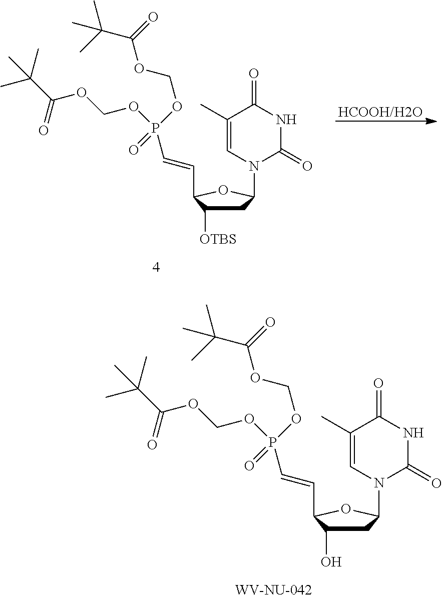

### 1. Preparation of Compound 1B

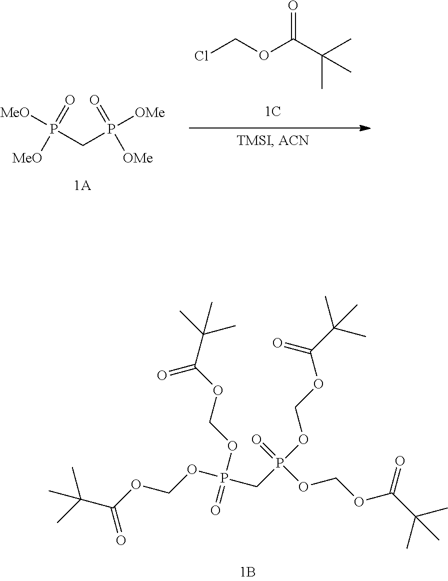

A mixture of compound 1A (47 g, 202.49 mmol), compound 1C (152.48 g, 1.01 mol, 146.61 mL), TBAI (74.79 g, 202.49 mmol) in ACN (400 mL), then the mixture was stirred and reflux at 85° C. for 15 hr. The mixture was added compound 1C (61 g) and stirred at 85° C. for 15 hr. TLC showed compound 1A was consumed and new spot was detected. The mixture was diluted with Ethyl acetate (300 mL) and H₂O (300 mL), and extracted with Ethyl acetate (300 mL*3). The combined organic layers were dried over Na₂SO₄, filtered and concentrated under reduced pressure to give a crude. The crude was purified by column chromatography (SiO₂, Petroleum ether:Ethyl acetate=10:1, 5:1, 3:1 to 1:1). Compound 1B (106 g, 82.75% yield) was obtained as a white solid.

¹H NMR (400 MHz, CHLOROFORM-d) 6=5.77-5.68 (m, 8H), 2.78-2.59 (m, 2H), 1.25 (s, 36H).

TLC (Petroleum ether:Ethyl acetate=1:1), R_{f}=0.43.

### 2. Preparation of Compound 2

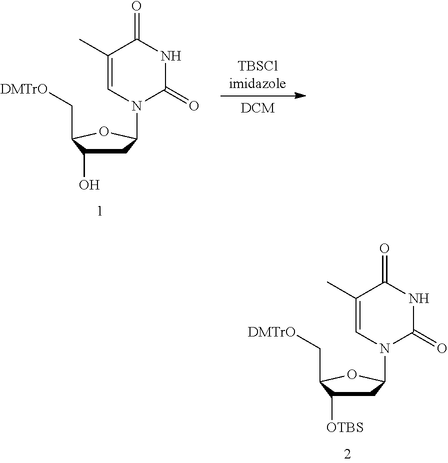

Three batches: To a solution of compound 1 (234 g, 429.68 mmol) and imidazole (87.75 g, 1.29 mol) in DCM (2 L) was added TBSCl (97.14 g, 644.52 mmol, 78.98 mL). The mixture was stirred at 15° C. for 16 hr. TLC showed compound 1 was consumed. Three batches mixture was combined and washed with sat. NaHCO₃ (aq., 4 L * 2), the combined aqueous was extracted with EtOAc (3 L*2), the combined organic was dried over Na₂SO₄, filtered and concentrated to give crude. Compound 2 (850 g, crude) was obtained as a yellow oil.

TLC (Petroleum ether:Ethyl acetate=1:1) R_{f}=0.39.

### 3. Preparation of WV-NU-041

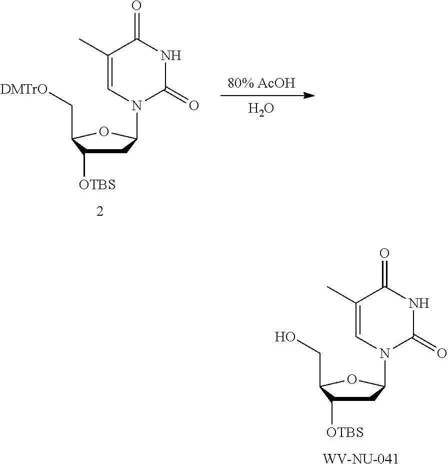

A solution of compound 2 (400 g, 607.11 mmol) in CH₃COOH (1200 mL) and H₂O (300 mL) and stirred at 15° C. for 16 hr. TLC showed compound 2 was partly remained and new spot was detected. The reaction suspension liquid was filtered to remove white solid, then filtrate was added to ice water (2 L), then white solid was appeared and filtered to give crude. The aqueous layers were extracted with EtOAc (2 L * 4). The combined organic layers were washed with sat. NaHCO₃ (aq., 1 L), dried over Na₂SO₄, filtered and combined with above crude, concentrated under reduced pressure to give a crude. The crude were purified by column chromatography (SiO₂, Petroleum ether:Ethyl acetate=5:1, 3:1, 1:1 to 0:1). Compound WV-NU-041 (100 g, 46.20% yield) was obtained as a yellow solid.

¹HNMR (400 MHz, CDCl₃) Shift=9.54 (br s, 1H), 7.42 (s, 1H), 6.16 (t, J=6.7 Hz, 1H), 4.51-4.46 (m, 1H), 3.96-3.87 (m, 2H), 3.82-3.68 (m, 1H), 2.32 (td, J=6.8, 13.5 Hz, 1H), 2.21 (ddd, J=3.7, 6.4, 13.2 Hz, 1H), 1.89 (s, 3H), 0.88 (s, 9H), 0.08 (s, 6H).

¹³CNMR (101 MHz, CDCl₃) Shift=164.30, 150.50, 137.15, 110.90, 87.60, 86.67, 71.55, 61.86, 40.53, 25.69, 20.72, 17.92, 12.44, −4.72, −4.88

LCMS: M+Na+=379.2, Purity: 96.33%.

TLC (Petroleum ether:Ethyl acetate=1:1) R_{f}=0.24.

### 4. Preparation of Compound 3

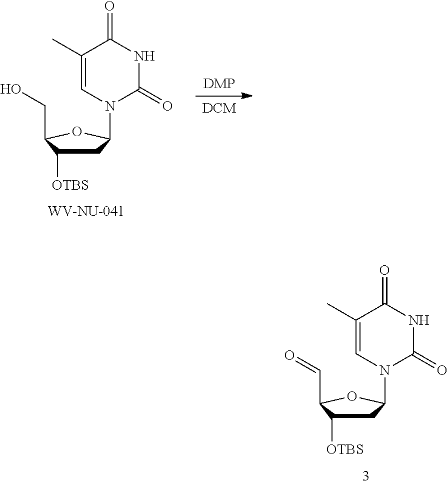

To a solution of compound WV-NU-041 (40 g, 112.21 mmol) in DCM (300 mL) was added DMP (66.63 g, 157.09 mmol) in portions at 0° C. The mixture was stirred at 0° C. for 1 hr, and then warmed to 25° C. and stirred at 25° C. for 2 hr. TLC showed most of compound WV-NU-041 was disappeared and new spot was found. The mixture was diluted with ethyl acetate (500 mL) and filtrated through a short silica gel pad (SiO₂, 200 g) using ethyl acetate (500 mL). The mixture was added 5% Na₂SO₃/sat. NaHCO₃ (1:1, 500 mL, aq.) at 0° C., the mixture was extracted with Ethyl acetate (300 mL*2), the combined organic was dried over Na₂SO₄, filtered and concentrated to get crude. Compound 3 (39 g, crude) was obtained as a white solid.

LCMS: M+H⁺=354.9.

TLC (Petroleum ether:Ethyl acetate=1:3) R_{f}=0.36.

### 5. Preparation of Compound 4

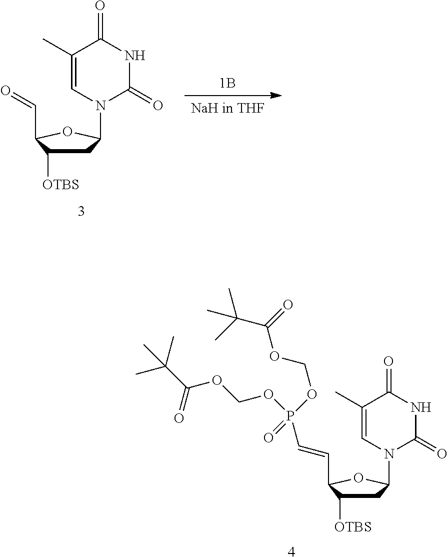

To a solution of NaH (10.62 g, 265.58 mmol, 60% purity) in THF (400 mL) was added compound 1B (140 g, 221.32 mmol) in THF (600 mL) at −70° C.-−60° C. under N₂ over 30 min. The reaction mixture was stirred for 30 min at −70° C.-−60° C. under N₂. To the above mixture was added a solution of compound 3 (31.38 g, 88.53 mmol) in THF (400 mL) at −70° C.-−60° C. under N₂ over 30 min. The mixture was stirred at −70° C.-−60° C. for 1 hr under N₂, 0° C. for 1 hr and then 18° C. for 2 hr. TLC showed compound 3 was consumed. The mixture was added to sat.NH₄Cl (1000 mL, aq.) at 0° C., extracted with Ethyl acetate (1000 mL*3). The combined organic layers were concentrated under reduced pressure to give a residue. The residue was purified by column chromatography (SiO₂, Petroleum ether:Ethyl acetate=5:1, 3:1 to 1:1). Compound 4 (25 g, 42.74% yield) was obtained as a colorless oil.

TLC (Petroleum ether:Ethyl acetate=1:1) R_{f}=0.18.

### 6. Preparation of WV-NU-042, (5′-(E)-(POM)2-VPdT)

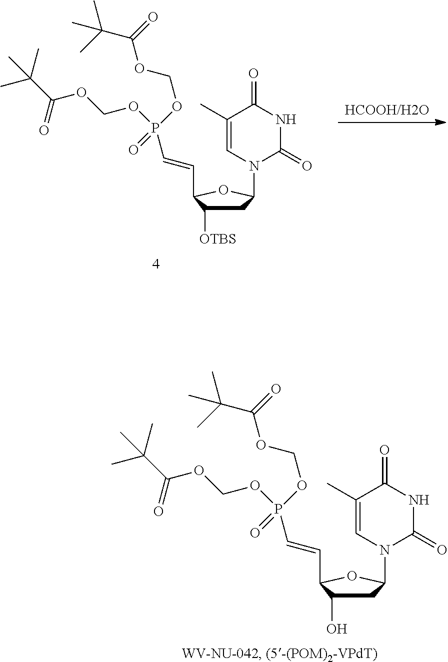

A solution of compound 4 (35.5 g, 53.73 mmol) in HCOOH (150 mL) and H₂O (150 mL) at 0° C., the mixture was stirred at 0-15° C. for 16 hr. TLC and LCMS showed compound 4 was consumed and new spot was detected. The mixture was concentrated under reduced pressure to give a residue at 30° C. water bath. The residue was purified by MPLC (SiO₂, Ethyl acetate/Petroleum ether=20%, 50%, 100%). Compound WV-NU-042, (5′-(E)-(POM)2-VPdT) (18.6 g, 63.35% yield) was obtained as a yellow gum.

¹H NMR (400 MHz, CDCl₃) Shift=8.92 (s, 1H), 7.06 (d, J=0.9 Hz, 1H), 7.01-6.84 (m, 1H), 6.28 (t, J=6.6 Hz, 1H), 6.08-5.90 (m, 1H), 5.70-5.57 (m, 3H), 5.54 (dd, J=5.1, 12.3 Hz, 1H), 4.39-4.25 (m, 2H), 3.67 (br s, 1H), 2.35 (ddd, J=4.7, 6.6, 13.8 Hz, 1H), 2.15 (td, J=6.8, 13.7 Hz, 1H), 1.91-1.80 (m, 3H), 1.15 (d, J=2.7 Hz, 18H).

¹³C NMR (101 MHz, CDCl₃) Shift=177.20, 176.89, 163.46, 150.33, 149.84, 149.78, 135.18, 118.20, 116.29, 111.74, 85.71, 85.48, 84.93, 81.56, 73.94, 60.40, 39.09, 38.76, 26.83, 26.81, 21.04, 14.19, 12.61.

³¹P NMR (162 MHz CDCl₃,) Shift=17.05.

LCMS: M+H⁺=547.2, purity: 90.718%.

### Example 27: Synthesis of Abasic 5′-Vinyl Phosphonates (WV-RA-009) and 5′-Vinyl Phosphonates-3′-CNE Phosphoramidite (WV-RA-009-CNE)

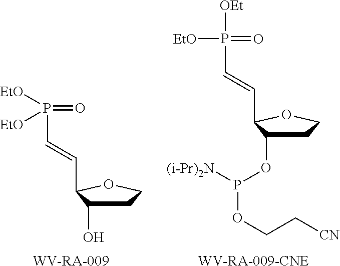

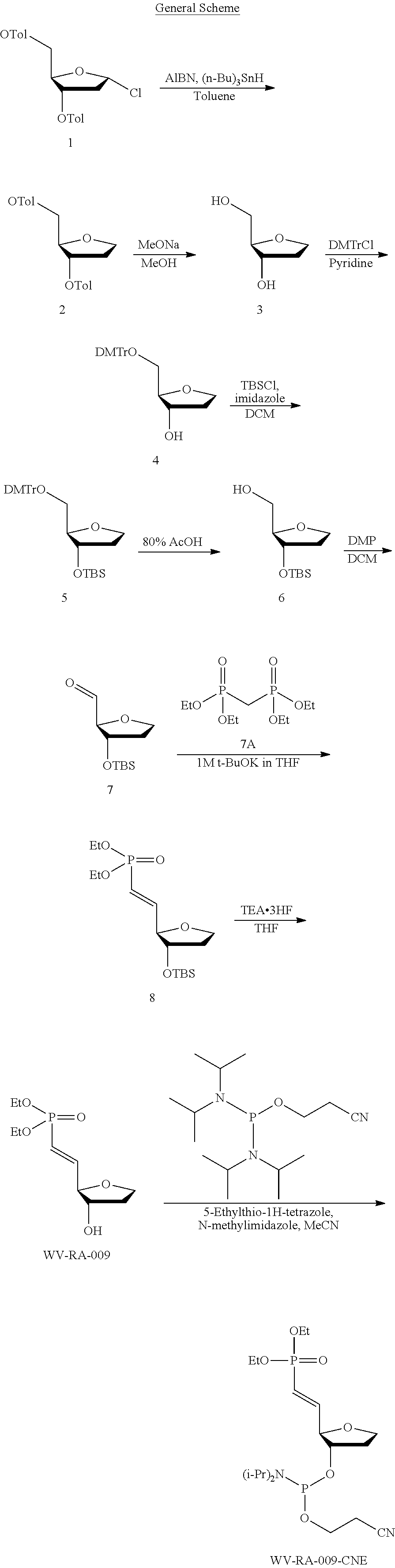

### 1. Preparation of Compound 2

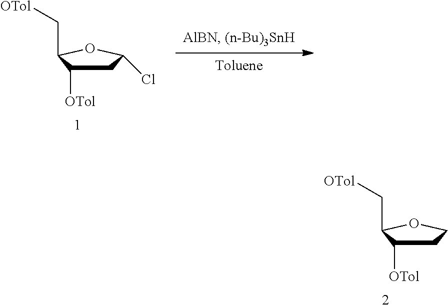

Three batches: The compound 1 (100 g, 257.17 mmol) was dissolved in dry toluene (1500 mL), and AIBN (1.58 g, 9.64 mmol) and (n-Bu)₃SnH (74.85 g, 257.17 mmol) were added. The solution was heated to 80° C. for 12 h. TLC showed little of compound 1 was still remained and a new spot was found. The three batches were combined for work up. The mixture was evaporated to dryness to give (270 g, crude) as a yellow oil. The crude mixture (315 g) was purified by silica gel chromatography (Petroleum ether/Ethyl acetate=100/1, 50/1) to get compound 2 (120 g, 38.10% yield) as a yellow oil and 120 g crude need further purification.

TLC (Petroleum ether:Ethyl acetate=3:1), R_{f}=0.63

### 2. Preparation of Compound 3

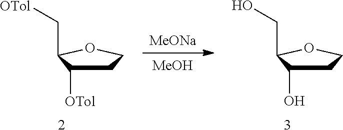

Three batches: To a solution of compound 2 (115 g, 324.50 mmol) in MeOH (1.2 L) was added NaOMe (52.59 g, 973.49 mmol). The mixture was stirred at 25° C. for 3 hr. LCMS and TLC showed compound 2 was consumed and TLC showed a new spot was found. Three batches were combined for work up. NH₄Cl (169 g) was added and the mixture was concentrated to get the compound 3 (115 g, crude) as a yellow oil.

TLC (Ethyl acetate:Methanol=10:1), R_{f}=0.21

### 3. Preparation of Compound 4

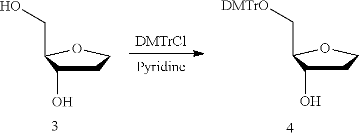

To a solution of compound 3 (55 g, 465.59 mmol) in pyridine (550 mL) was added DMTCl (189.30 g, 558.70 mmol). The mixture was stirred at 25° C. for 12 h. LCMS showed the compound 3 was consumed and the desired substance was found. Water (500 mL) was added and the mixture was extracted with EtOAc (500 mL*2). The combined organic was dried over sodium sulfate, filtered and concentrated to get the crude. The mixture was purified by silica gel chromatography (Petroleum ether:Ethyl acetate=10:1, 3:1, 1:1, 5% TEA) to get compound 4 (110 g, 56.19% yield) as a yellow oil.

TLC (Petroleum ether:Ethyl acetate=1:1), R_{f}=0.43

### 4. Preparation of Compound 5

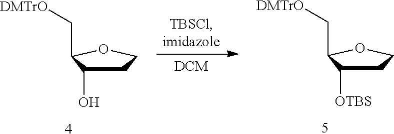

Two batches: To a solution of compound 4 (55 g, 130.80 mmol) and imidazole (26.71 g, 392.39 mmol) in DCM (600 mL) was added TBSCl (29.57 g, 196.20 mmol). The mixture was stirred at 25° C. for 12 h. TLC showed the compound 4 was consumed and a new spot was found. The two batches were combined for work up. Water (500 mL) was added and extracted with DCM (200 mL*2). The combined organic was dried over Na₂SO₄, filtered and concentrated to get the compound 5 (139 g, crude) as a yellow oil TLC (Petroleum ether:Ethyl acetate=5:1), R_{f}=0.47

### 5. Preparation of Compound 6

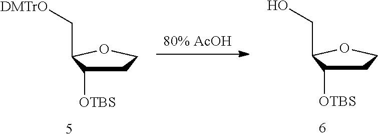

A solution of compound 5 (139 g, 259.93 mmol) in the mixture of HOAc (560 mL) and H₂O (140 mL) was stirred at 25° C. for 12 hr. TLC showed compound 5 was consumed. The mixture was poured into ice-water (500 mL), and the NaHCO₃ solid was added until pH=7, and the residue was extracted with EtOAc (300 mL*3). The combined organic was washed with brine (300 mL), dried over Na₂SO₄, filtered and concentrated to get the crude. The mixture was purified by silica gel chromatography (Petroleum ether/Ethyl acetate=50/1, 5/1, 3:1) to get the compound 6 (35 g, 57.94% yield) as a yellow oil.

¹HNMR (400 MHz, CDCl₃) δ=4.23 (td, J=3.8, 6.6 Hz, 1H), 3.97 (dd, J=5.4, 8.0 Hz, 2H), 3.79-3.68 (m, 2H), 3.61-3.50 (m, 1H), 2.06-2.01 (m, 1H), 1.91-1.81 (m, 1H), 0.89 (s, 8H), 0.08 (s, 6H)

TLC (Petroleum ether:Ethyl acetate=5:1), R_{f}=0.25

### 6. Preparation of Compound 7

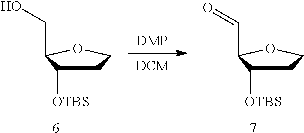

Two batches: To a solution of compound 6 (14.5 g, 62.39 mmol) in DCM (150 mL) was added DMP (31.76 g, 74.87 mmol). The reaction was stirred at 25° C. for 2 hr. TLC showed compound 6 was consumed. The two batches were combined for work up. The mixture was poured into the mixture of sat. NaHCO₃ (750 mL) and sat. Na₂SO₃ (750 mL). The mixture was extracted with DCM (500 mL*2); the combined organic was washed with brine (500 mL), dried over Na₂SO₄, filtered and concentrated to get the compound 7 (28.7 g, crude) as a yellow oil.

TLC (Petroleum ether:Ethyl acetate=3:1), R_{f}=0.43

### 7. Preparation of Compound 8

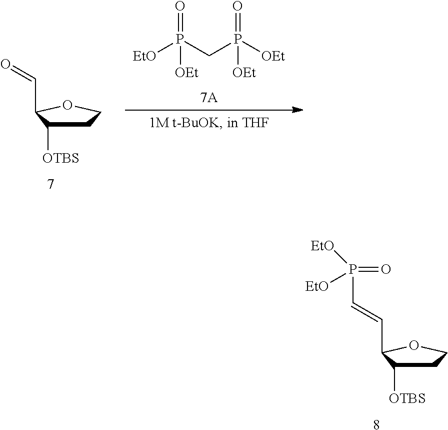

To a solution of compound 7A (43.09 g, 149.50 mmol) in THF (100 mL) was added t-BuOK (1 M, 149.50 mL) at 0° C., and stirred at 0° C. for 10 min, then warmed up to 25° C. for 30 min. The solution of compound 7 (28.7 g, 124.58 mmol) in THF (100 mL) was added to the above solution at 0° C. The reaction mixture was stirred at 0° C. for 1 h, and then allowed to warm up to 25° C. in 80 min. TLC showed compound 7 was consumed. To the reaction mixture water (100 mL) was added and extracted with EtOAc (100 mL*4). The organic phase was dried (Na₂SO₄), filtered and concentrated to give compound 8 (45 g, crude) as a colorless oil.

The mixture was purified by silica column (Petroleum ether/Ethyl acetate=10/1, 3/1) to get compound 8 (18 g, 40.00% yield) as a yellow oil.

¹HNMR (400 MHz, CDCl₃) δ=6.86-6.68 (m, 1H), 6.08-5.82 (m, 1H), 4.32-4.26 (m, 1H), 4.20-4.13 (m, 1H), 4.12-3.99 (m, 6H), 2.04-1.93 (m, 1H), 1.88-1.79 (m, 1H), 1.59 (s, 2H), 1.33 (t, J=7.1 Hz, 6H), 0.90 (s, 9H), 0.15-−0.02 (m, 6H)

TLC: (Petroleum ether:Ethyl acetate=1:1), Rf=0.15

### 8. Preparation of Compound WV-RA-009

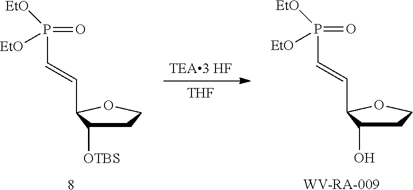

To a solution of compound 8 (20 g, 54.87 mmol) in THF (200 mL) was added 3HF·TEA (35.38 g, 219.49 mmol). The mixture was stirred at 25° C. for 2 hr. TLC showed compound 8 was consumed, a new spot was found. NaHCO₃ (300 mL, aq.) was added, and extracted with DCM (200 mL*5). The combined organic was dried over Na₂SO₄, filtered and concentrated to get the crude. The mixture was purified by silica column (Petroleum ether/Ethyl acetate=10/1, 3/1, 0:1) to get the WV-RA-009 (11.5 g, 82.14% yield) as a colorless oil.

¹HNMR (400 MHz, CDCl₃) δ=6.90-6.78 (m, 1H), 6.08-5.84 (m, 1H), 4.41-4.36 (m, 1H), 4.23 (td, J=3.1, 6.0 Hz, 1H), 4.12-4.00 (m, 6H), 3.44 (br s, 1H), 2.13-1.99 (m, 1H), 1.97-1.89 (m, 1H), 1.32 (dt, J=1.4, 7.1 Hz, 6H)

¹³CNMR (101 MHz CDCl₃,) δ=150.69, 150.63, 117.25, 115.37, 85.79, 85.58, 75.54, 67.31, 61.92 (t, J=6.2 Hz, 1C), 34.07, 16.35, 16.30

³¹P NMR (162 MHz, CDCl₃) δ=18.65 (s, 1P)

LCMS: (M+H⁺): 251.1, LCMS purity: 100% (ELSD).

TLC (Petroleum ether:Ethyl acetate=0:1), R_{f}=0.15

### 9. Preparation of Compound WV-RA-009-CNE Phosphoramidite

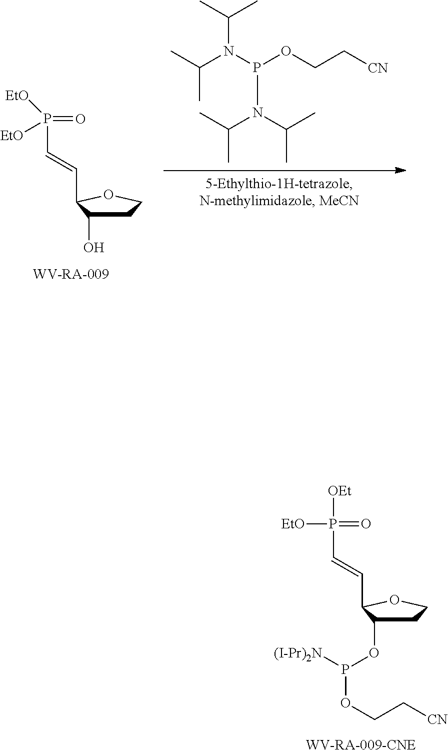

The compound WV-RA-009 (4.5 g, 17.98 mmol) was dried by azeotropic distillation on a rotary evaporator with toluene (20 mL*3).

To a solution of compound WV-RA-009 (4.5 g, 17.98 mmol) in DMF (32 mL) were added N-methylimidazole (2.95 g, 35.97 mmol) and 5-ethylsulfanyl-2H-tetrazole (2.34 g, 17.98 mmol), then 3-bis(diisopropylamino)phosphanyloxypropanenitrile (8.13 g, 26.98 mmol) was dropped. The mixture was stirred at 25° C. for 2 hr. TLC showed WV-RA-009 was consumed and a new spot was found. The mixture was poured into the sat. NaHCO₃ (200 mL) slowly, and the mixture was extracted with EtOAc (100 mL*3). The combined organic was dried over Na₂SO₄, filtered and concentrated to get the crude. The residue was purified by silica gel chromatography (Petroleum ether/Ethyl acetate=10/1, 3/1, 1/1, 5% TEA) for two times to get WV-RA-009-CNE (3 g, 33.90% yield, 91.55% purity) as a colorless oil.

¹HNMR (400 MHz CDCl₃,) δ=6.90-6.64 (m, 1H), 6.09-5.82 (m, 1H), 4.47 (br d, J=17.0 Hz, 1H), 4.30-4.19 (m, 1H), 4.12-3.95 (m, 6H), 3.86-3.69 (m, 2H), 3.64-3.52 (m, 2H), 2.68-2.55 (m, 2H), 2.09-1.89 (m, 2H), 1.29 (dt, J=2.2, 7.1 Hz, 7H), 1.23-1.11 (m, 14H)

³¹PNMR (162 MHz, CDCl₃) δ=148.22 (s, 1P), 148.11 (s, 1P), 148.32-147.99 (m, 1P), 30.79 (s, 1P), 18.38 (s, 1P), 18.33 (s, 1P), 18.22 (s, 1P)

¹³CNMR (101 MHz, CDCl₃) δ=149.83 (dd, J=5.9, 11.7 Hz, 1C), 118.10, 117.88, 117.55, 117.51, 116.23, 116.01, 84.75 (br dd, J=19.1, 21.3 Hz, 1C), 84.70 (br t, J=20.9 Hz, 1C), 67.61, 67.58, 61.76 (br t, J=3.7 Hz, 1C), 58.37, 58.29, 58.18, 58.10, 43.23 (dd, J=2.9, 12.5 Hz, 1C), 33.23 (dd, J=4.0, 9.2 Hz, 1C), 24.60, 24.54, 24.51, 24.39, 23.88, 20.34 (dd, J=4.0, 7.0 Hz, 1C), 16.36, 16.29

LCMS: purity 91.55% (ELSD)

TLC (Petroleum ether:Ethyl acetate=0:1), Rf=0.43

### Example 28: Synthesis of Abasic 5′-(R)-Me-PO(OEt)₂-Phosphonate (WV-RA-010), and 5′-(R)-Me-PO(OEt)₂-Phosphonate-3′-CNE Phosphoramidite (WV-RA-010-CNE)

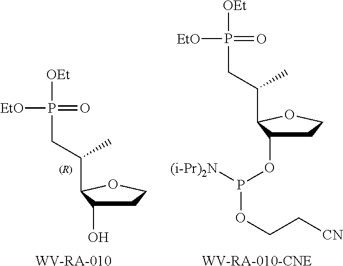

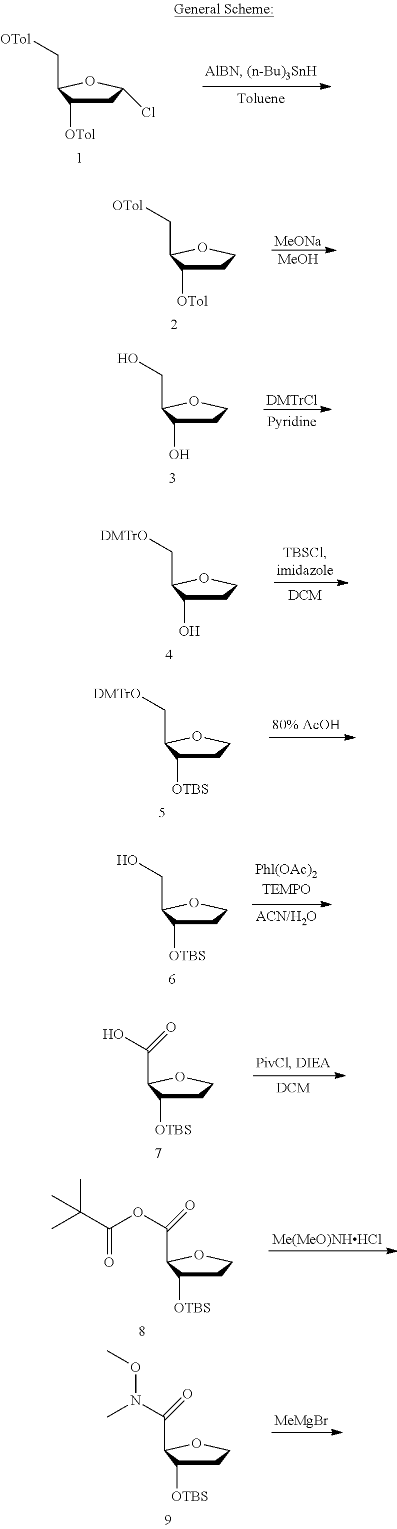

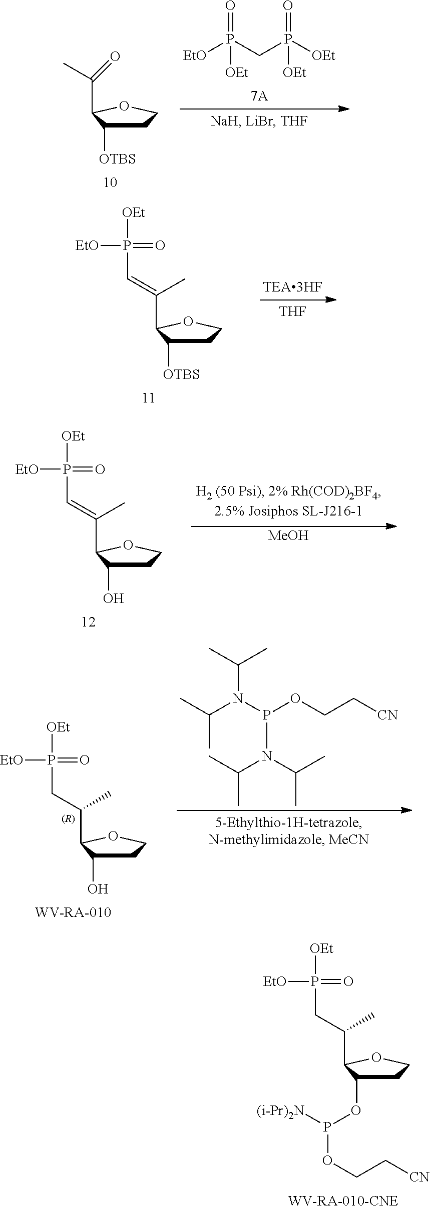

### 1. Preparation of Compound 2

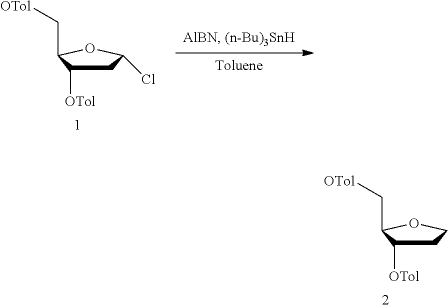

For three batches: The compound 1 (100 g, 257.17 mmol) was dissolved in dry toluene (1500 mL) and the AIBN (1.58 g, 9.64 mmol) and (n-Bu)₃SnH (74.85 g, 257.17 mmol) were added. The solution was heated to 80° C. for 12 h. TLC showed little of compound 1 still remained and a new spot was found. The three batches were combined for work up. The mixture was evaporated to dryness.

Purification: The crude mixture (315 g) was purified by silica gel chromatography (Petroleum ether/Ethyl acetate=100/1, 50/1) to get compound 2 (120 g, 38.10% yield) as a yellow oil.

¹H NMR (400 MHz, CDCl₃) δ=7.98-7.91 (m, 4H), 7.29-7.21 (m, 4H), 5.48 (td, J=2.2, 6.4 Hz, 1H), 4.55-4.46 (m, 2H), 4.38 (dt, J=2.6, 4.6 Hz, 1H), 4.21-4.13 (m, 1H), 4.05 (dt, J=6.1, 9.2 Hz, 1H), 2.42 (d, J=5.6 Hz, 7H), 2.20 (tdd, J=2.8, 5.6, 13.5 Hz, 1H)

TLC (Petroleum ether:Ethyl acetate=5:1), Rf=0.47

### 2. Preparation of Compound 3

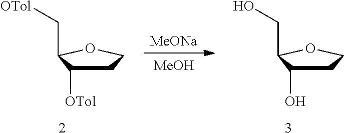

For three batches: To a solution of compound 2 (115 g, 324.50 mmol) in MeOH (1.2 L) was added NaOMe (52.59 g, 973.49 mmol). The mixture was stirred at 25° C. for 3 h. LCMS and TLC showed compound 2 was consumed and a new spot was found. Three batches were combined for work up. NH₄Cl (169 g) was added and the mixture was concentrated to get the compound 3 (115 g, crude) as a yellow oil.

TLC (Ethyl acetate:Methanol=10:1), R_{f}=0.21

### 3. Preparation of Compound 4

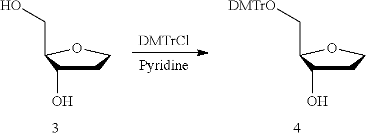

To a solution of compound 3 (60 g, 507.91 mmol) in pyridine (600 mL) was added DMTCl (206.51 g, 609.49 mmol). The mixture was stirred at 25° C. for 12 h. LCMS showed compound 3 was consumed and the desired substance was found. Water (600 mL) was added and the mixture was extracted with EtOAc (600 mL*2). The combined organic was dried over sodium sulfate, filtrated and concentrated to get the crude. The crude was purified by column chromatography (SiO₂, Petroleum ether/Ethyl acetate=15/1 to 1/1) to get compound 4 (89 g, 41.78% yield) as a yellow oil.

LCMS: NEG (M−H⁺), 419.1

TLC (Petroleum ether:Ethyl acetate=1:1), Rf=0.43

### 4. Preparation of Compound 5

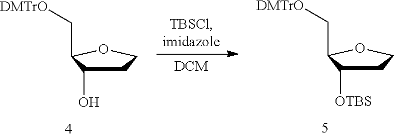

For two batches: To a solution of compound 4 (44.5 g, 105.83 mmol) and imidazole (21.61 g, 317.48 mmol) in DCM (500 mL) was added TBSCl (23.93 g, 158.74 mmol), and the mixture was stirred at 25° C. for 12 h. TLC showed compound 4 was consumed and a new spot was found. The two batches were combined for work up. Water (500 mL) was added and extracted with DCM (200 mL*2). The combined organic was dried over Na₂SO₄, filtered and concentrated to get the compound 5 (113 g, crude) as a yellow oil

TLC (Petroleum ether:Ethyl acetate=5:1), Rf=0.47

### 5. Preparation of Compound 6

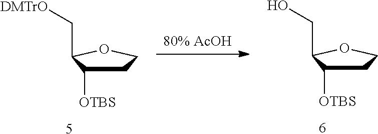

For two batches: To a solution of compound 5 (56.5 g, 105.66 mmol) in the mixture of HOAc (240 mL) and H₂O (60 mL), the residue was stirred at 25° C. for 12 h. TLC showed compound 5 was consumed. The two batches were combined for workup. The mixture was poured into ice-water (500 mL) and the NaHCO₃solid was added until pH=7, and the residue was extracted with EtOAc (300 mL*3), the combined organic was washed with brine (300 mL), dried over Na₂SO₄, filtered and concentrated to get the crude. The mixture was purified by silica gel chromatography (Petroleum ether/Ethyl acetate=50/1, 5/1, 3:1) to get the compound 6 (28 g, 57.03% yield) as a yellow oil.

TLC (Petroleum ether:Ethyl acetate=5:1), Rf=0.25

### 6. Preparation of Compound 7

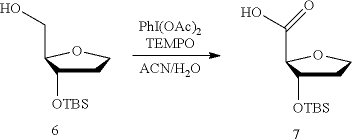

To a solution of compound 6 (13 g, 55.94 mmol) in MeCN (150 mL) and H₂O (150 mL) was added PhI(OAc)₂ (39.64 g, 123.07 mmol) and TEMPO (1.76 g, 11.19 mmol). The mixture was stirred at 25° C. for 3 h. TLC showed compound 6 was consumed and a new spot was found. The mixture was concentrated to get the compound 7 (27 g, crude) as a yellow oil.

TLC (Petroleum ether:Ethyl acetate=3:1), Rf=0.04

### 7. Preparation of Compound 8

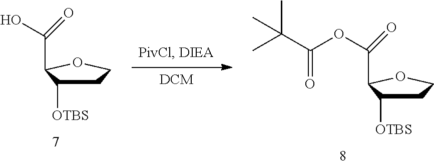

For two batches: To a solution of compound 7 (13.5 g, 54.79 mmol) in DCM (135 mL) was added DIEA (14.16 g, 109.59 mmol) and 2,2-dimethylpropanoyl chloride (8.59 g, 71.23 mmol). The mixture was stirred at 0° C. for 0.5 h. TLC showed compound 7 was consumed and anew spot was found. Compound 8 (36.2 g, crude) as a yellow solution in DCM (135 mL) was used for next step directly.

TLC (Petroleum ether:Ethyl acetate=1:1), Rf=0.22

### 8. Preparation of Compound 9

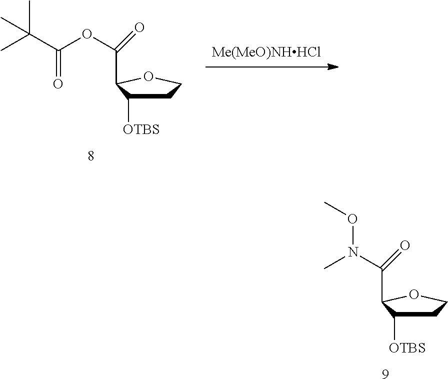

For two batches: The mixture compound 8 (18.1 g, 54.77 mmol) in DCM (135 mL) from the last step was added TEA (16.63 g, 164.30 mmol, 22.87 mL) and N— methoxymethanamine;hydrochloride (8.01 g, 82.15 mmol), and the mixture was stirred at 0° C. for 1 h. LCMS showed the starting material was consumed and the desired substance was found. The two batches were combined for work up. The mixture was washed with HCl (1N, 100 mL) and then aqueous NaHCO₃ (100 mL), the organic was dried over Na₂SO₄and filtered to get the crude. The mixture was purified by silica gel chromatography (Petroleum ether/Ethyl acetate=10/1, 3/1) to get compound 9 (15.8 g, 50.97% yield) as a yellow oil.

LCMS: (M+H⁺): 290.1

### 9. Preparation of Compound 10

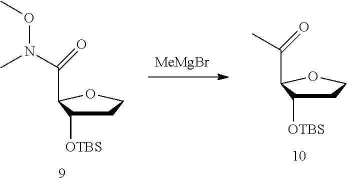

To a solution of compound 9 (15.8 g, 54.59 mmol) in THF (180 mL) was dropped MeMgBr (3 M, 54.59 mL) at 0° C., and the mixture was stirred at 0° C. for 1 hr. TLC showed compound 9 was consumed. The mixture was poured into sat.NH₄Cl (200 mL) and the mixture was extracted with EtOAc (150 mL*3), the combined organic was dried over Na₂SO₄, filtered and concentrated to get the crude. The mixture was purified by silica gel chromatography (Petroleum ether/Ethyl acetate=20/1, 5/1) to get compound 10 (12 g, 85.11% yield) as a yellow oil.

¹HNMR (400 MHz, CDCl₃) δ=4.47-4.41 (m, 1H), 4.18 (d, J=2.5 Hz, 1H), 4.11-4.01 (m, 2H), 2.19 (s, 3H), 2.01-1.75 (m, 2H), 0.90 (s, 10H), 0.11 (d, J=3.5 Hz, 6H)

TLC (Petroleum ether:Ethyl acetate=3:1), Rf=0.76

### 10. Preparation of Compound 11

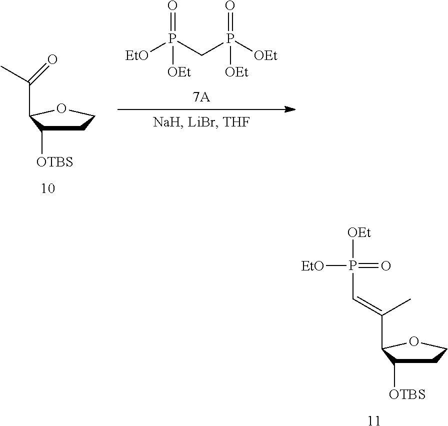

To a solution of NaH (7.92 g, 198.03 mmol, 60% purity) in THF (170 mL) was added compound 7A (57.08 g, 198.03 mmol) in THF (110 mL) at 0° C. The reaction mixture was warmed up to 20° C., and stirred for 1 hr. A solution of LiBr (17.20 g, 198.03 mmol) in THF (100 mL) was added and the resultant slurry was stirred, and then cooled to 0° C. To the above mixture was added a solution of compound 10 (11 g, 45.01 mmol) in THF (100 mL) at 0° C. The mixture was stirred at 0-20° C. for 1 hr. TLC showed compound 10 was consumed. The resulting mixture was diluted with water (500 mL), extracted with EtOAc (300 mL*3). The combined organic layers were dried over anhydrous Na₂SO₄, filtered and concentrated to afford a yellow oil. The residue was purified by silica gel chromatography (Petroleum ether/Ethyl acetate=1 0/1, 3/1) to get compound 11 (16 g, 86.49% yield) as a yellow oil.

¹HNMR (400 MHz, CDCl₃) δ=5.73 (td, J=1.1, 18.7 Hz, 1H), 4.19-3.94 (m, 9H), 2.09 (dd, J=0.8, 3.3 Hz, 3H), 2.01-1.90 (m, 1H), 1.84-1.75 (m, 1H), 1.40-1.27 (m, 8H), 0.93-0.84 (m, 10H), 0.13-0.00 (m, 6H)

TLC (Petroleum ether:Ethyl acetate=1:1), Rf=0.20

### 11. Preparation of Compound 12

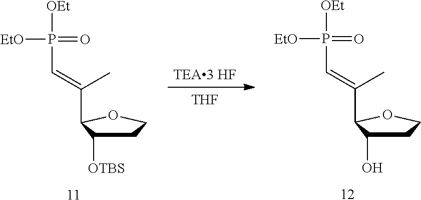

A mixture of compound 11 (15 g, 39.63 mmol) in THF (150 mL) was added 3HF·TEA (25.55 g, 158.51 mmol), and then the mixture was stirred at 20° C. for 12 hr under N₂ atmosphere. TLC showed compound 11 was consumed. Sat. NaHCO₃ was added to the mixture until pH=7, and the residue was extracted with DCM (150 mL*3), the combined organic was dried over Na₂SO₄, filtered and concentrated to get the crude. The residue was purified by silica gel chromatography (Petroleum ether/Ethyl acetate=5/1, 0/1, Ethyl acetate:Dichloromethane=10:1) to get compound 12 (8.8 g, 80.00% yield) as a yellow oil.

¹HNMR (400 MHz, CDCl₃) δ=5.73 (td, J=1.1, 18.7 Hz, 1H), 4.19-3.94 (m, 8H), 2.09 (dd, J=0.8, 3.3 Hz, 3H), 2.01-1.90 (m, 1H), 1.84-1.75 (m, 1H), 1.33-1.30 (m, 6H)

³¹PNMR (162 MHz, CDCl₃) δ=18.71

TLC (Ethyl acetate:Methanol=0:1), R_{f}=0.20.

### 12. Preparation of Compound WV-RA-010

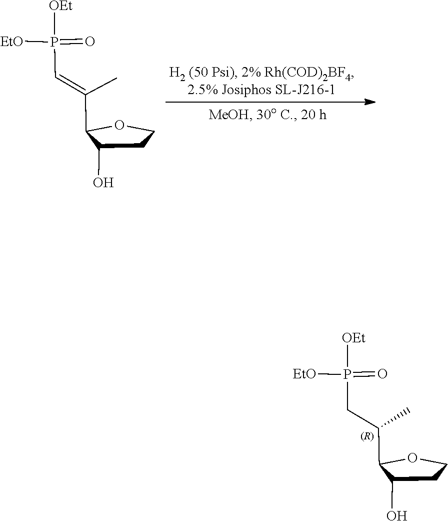

To a solution of compound 12 (8.7 g, 32.92 mmol), (1Z,5Z)-cycloocta-1,5-diene; rhodium(1+);tetrafluoroborate (534.76 mg, 1.32 mmol), zinc; trifluoromethanesulfonate (4.79 g, 13.17 mmol) in MeOH (160 mL) was added Josiphos SL-J216-1 (987.42 mg, 1.51 mmol) under N₂. The suspension was degassed under vacuum and purged with H₂ several times. The mixture was stirred under H₂ (50 psi) at 30° C. for 12 h. TLC showed the reaction was complete. The mixture was concentrated to get the crude. The residue was purified by silica gel chromatography (Petroleum ether/Ethyl acetate=20/1, 1/9, Ethyl acetate:Methanol=20:1) to get WV-RA-010 (8 g, 91.95% yield) as a yellow oil.

¹HNMR (400 MHz, CDCl₃) δ=4.27-4.01 (m, 5H), 3.99-3.81 (m, 2H), 3.61-3.41 (m, 2H), 2.23-1.86 (m, 4H), 1.80-1.63 (m, 1H), 1.34 (t, J=7.0 Hz, 6H), 1.12 (d, J=6.6 Hz, 3H)

¹³CNMR (101 MHz, CDCl₃) δ=89.97, 89.85, 74.18, 66.68, 61.92, 35.71, 31.49, 31.46, 29.95, 28.55, 17.50, 17.43, 16.42, 16.36

³¹PNMR (162 MHz, CDCl₃) δ=32.07

LCMS: ELSD (M+H⁺), 267.1, 100% purity

TLC (Petroleum ether:Ethyl acetate=0:1), R_{f}=0.03; (Ethyl acetate:Methanol=10:1), Rf=0.35.

### 13. Preparation of Compound WV-RA-010-CNE Phosphoramidite

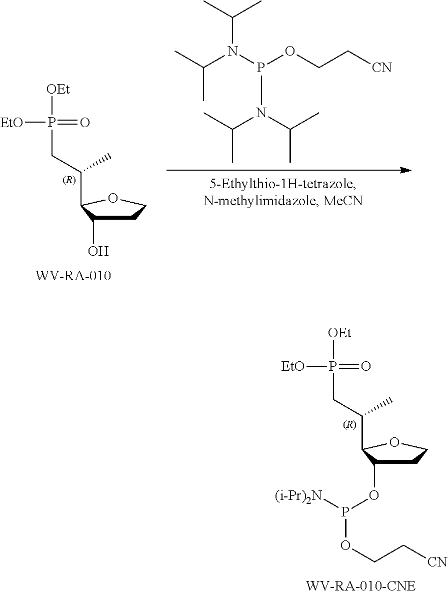

WV-RA-010 (3 g, 11.27 mmol) was dried by azeotropic distillation on a rotary evaporator with toluene (20 mL*3).To a solution of WV-RA-010 (3 g, 11.27 mmol) in DMF (24 mL) was added 1-methylimidazole (1.85 g, 22.53 mmol) and 5-ethylsulfanyl-2H-tetrazole (1.47 g, 11.27 mmol), then 3-bis(diisopropylamino)phosphanyloxypropanenitrile (5.09 g, 16.90 mmol) was dropped. The mixture was stirred at 25° C. for 1 h. TLC showed WV-RA-010 was consumed and a new spot was found. The mixture was poured into the sat. NaHCO₃ (100 mL) slowly and the mixture was extracted with Ethyl acetate (50 mL*3), the combined organic was dried over Na₂SO₄, filtered and concentrated to get the crude. The residue was purified by silica gel chromatography (Petroleum ether/Ethyl acetate=10/1, 3/1, 1/1, 5% TEA) to get WV-CA-010-CNE (1.7 g, 32.35% yield) as a colorless.

¹HNMR (400 MHz CDCl₃,) δ=4.29-4.18 (m, 1H), 4.17-4.02 (m, 4H), 4.01-3.49 (m, 7H), 2.71-2.59 (m, 2H), 2.24-2.08 (m, 1H), 2.07-1.92 (m, 3H), 1.74-1.49 (m, 2H), 1.37-1.28 (m, 7H), 1.24-1.15 (m, 12H), 1.10 (d, J=6.8 Hz, 3H)

¹³CNMR (101 MHz, CDCl₃) δ=117.58, 117.54, 89.31, 89.25, 75.58, 66.95, 61.36, 61.37, 58.07, 46.34, 46.30, 45.49, 45.45, 45.40, 43.20, 34.84, 30.87, 30.84, 30.81, 29.81, 29.79, 28.42, 28.38, 25.72, 24.54, 24.47, 24.39, 23.85, 23.15, 24.36, 22.98, 22.64, 24.29, 20.39, 20.31, 20.30, 20.23, 16.41, 16.35, 15.94, 15.40

³¹PNMR (162 MHz, CDCl₃) δ=148.02, 147.78, 31.73, 31.57 (s, 1P), 30.79 (s, 1P)

LCMS: ELSD, 96.42% purity

TLC (Petroleum ether:Ethyl acetate=0:1), Rf=0.43

### Example 29: Synthesis of 5′-®—C-M′-5′—ODMT′-2′-F-dU

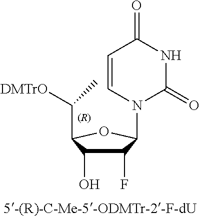

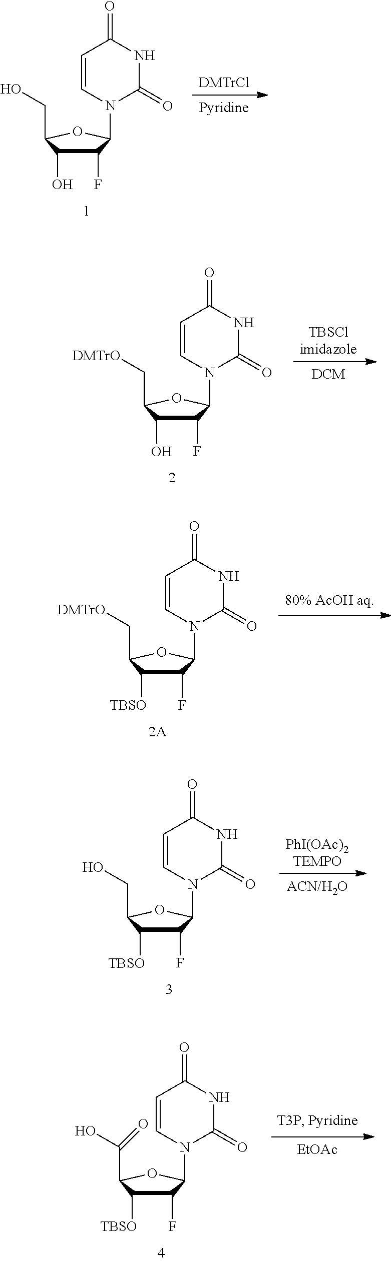

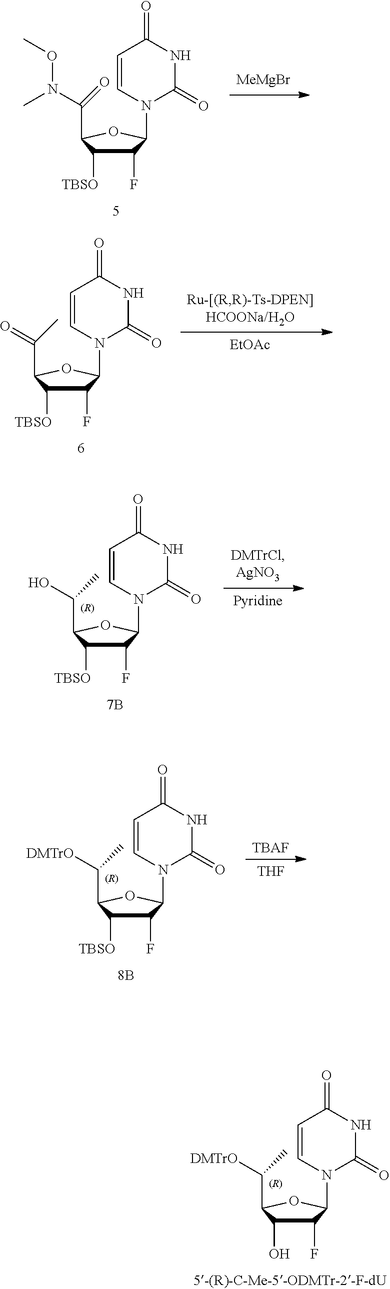

### 1. Preparation of Compound 2

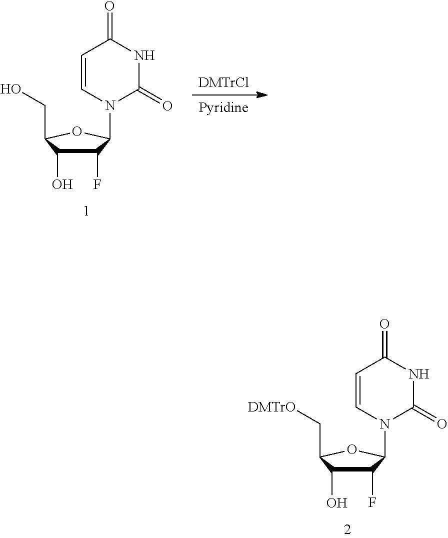

To a solution of compound 1 (100.00 g, 406.19 mmol) in pyridine (550.00 mL) was added DMTCl (165.16 g, 487.43 mmol). The mixture was stirred at 25° C. for 20 hr. TLC indicated compound 1 was consumed and one new spot formed. MeOH (300 mL) was added, and the reaction mixture was concentrated under reduced pressure to remove solvent. The residue was dissolved in EtOAc (500 mL) and washed with H₂O (500 mL*3). Dried over Na₂SO₄, filtered and concentrated under reduced pressure to give a residue. The crude product compound 2 (285.00 g, crude) was yellow solid, used into the next step without further purification.

TLC (Ethyl acetate:Petroleum ether=3:1, 5% TEA) R_{f}=0.40

### 2. Preparation of Compound 2A

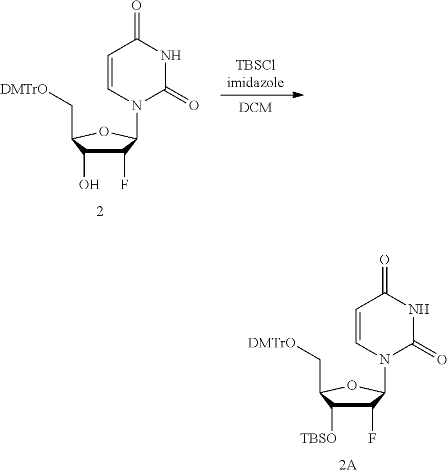

To a solution of compound 2 (222.82 g, 406.19 mmol) in DCM (500.00 mL) was added imidazole (41.48 g, 609.29 mmol) and TBSCl (91.83 g, 609.29 mmol, 74.66 mL). The mixture was stirred at 25° C. for 20 hour. TLC indicated compound 2 was consumed and one new spot formed. The reaction mixture was concentrated under reduced pressure to remove solvent. The residue was diluted with DCM (500 mL), and washed with H₂O mL (500 mL*3). Dried over Na₂SO₄, filtered and concentrated under reduced pressure to give a residue. The crude product compound 2A (330.00 g, crude) was white solid, used into the next step without further purification.

TLC (Ethyl acetate:Petroleum ether=3:1) R_{f}=0.65.

### 3. Preparation of Compound 3

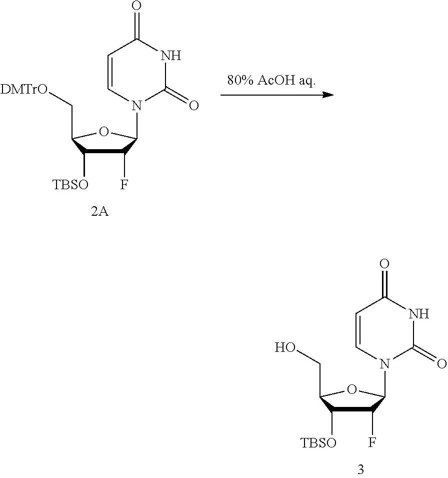

A solution of compound 2A (269.23 g, 406.19 mmol) in AcOH (400.00 mL) 80% aq., the mixture was stirred at 25° C. for 15 hour. TLC indicated compound 2A was remained a little and one new spot formed. The reaction mixture was quenched by sat. NaHCO₃ aq. until pH>7 at 25° C., and then diluted with EtOAc (500 mL) and extracted with EtOAc (500 mL*3). Dried over Na₂SO₄, filtered and concentrated under reduced pressure to give a residue. The residue was purified by column chromatography (SiO₂, Petroleum ether/Ethyl acetate=1/0 to 0/1 10% DCM). Got 60 g product and 130 g compound 2A. Compound 3 (60.00 g, 40.98% yield) was obtained as a white solid.

TLC (Ethyl acetate:Petroleum ether=3:1) R_{f}=0.45.

### 4. Preparation of Compound 4

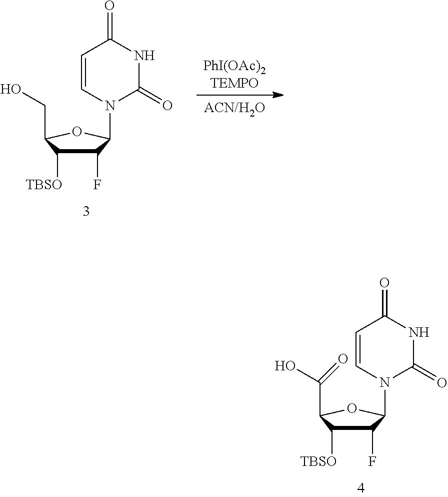

To a solution of compound 3 (30.00 g, 83.23 mmol) in MeCN (360.00 mL) and H₂O (360.00 mL) was added TEMPO (2.62 g, 16.65 mmol) and PhI(OAc)₂ (58.98 g, 183.10 mmol) at 25° C. in 3 hours. TLC indicated compound 3 was consumed and one new spot formed. The reaction mixture was concentrated under reduced pressure to remove lots of solvent. Filtered the solid and washed the solid with MeCN. The other liquid was concentrated under reduced pressure, then dissolved in sat. KOH (aq., 2M) to pH˜12, washed by EtOAc (200 mL*3), and then added HCl (aq. 1M) to pH˜3, filtered and concentrated as a yellow solid. Compound 4 (52.00 g, 138.87 mmol, 83.43% yield) was obtained as a yellow solid.

¹H NMR (400 MHz, DMSO-d₆) δ=7.96 (d, J=8.3 Hz, 1H), 5.99 (dd, J=3.1, 16.2 Hz, 1H), 5.71 (dd, J=2.2, 8.3 Hz, 1H), 5.34-5.03 (m, 1H), 4.70-4.48 (m, 1H), 4.30 (d, J=5.3 Hz, 1H), 0.86 (s, 9H), 0.08 (s, 6H).

LCMS: (M+H⁺): 374.9

TLC (Petroleum ether:Ethyl acetate=1:1, R_{f}=0)

### 6. Preparation of Compound 5

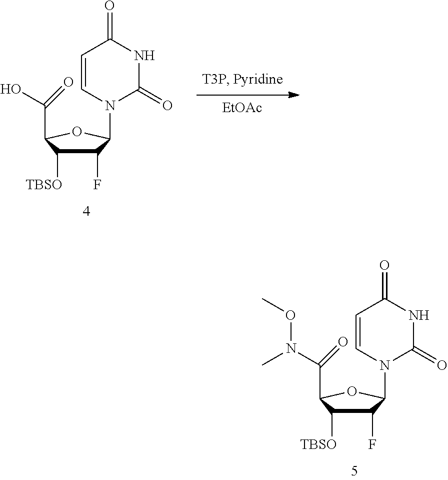

To a solution of compound 4 (26.00 g, 69.44 mmol) in pyridine (50.00 mL) was added N— methoxymethanamine hydrochloride (8.13 g, 83.33 mmol) and EtOAc (150.00 mL). The mixture was stirred at 0° C. then added T3P (46.40 g, 145.82 mmol, 43.36 mL) in N₂. The mixture was stirred at 0° C. in 3 h. TLC indicated compound 4 was consumed and one new spot formed. The resulting mixture was work up together with another batch (26 g scale). The resulting mixture was washed with HCl (1 M, 1.1 L), and the aqueous layer was extracted with DCM (1 L*2). The combined organic layers were washed with sat. Na₂CO₃ aq. until pH=12, dried over Na₂SO₄, filtered and concentrated to give a crude product. The residue was purified by column chromatography (SiO₂, Petroleum ether/Ethyl acetate=1/0 to 0:1) to get 52 g product. Compound 5 (26.00 g, 89.68% yield) was obtained as a yellow solid.

¹H NMR (400 MHz, CDCl₃) δ=8.91 (br s, 1H), 8.28 (d, J=8.2 Hz, 1H), 6.31 (dd, J=5.2, 11.6 Hz, 1H), 5.73 (dd, J=0.9, 8.2 Hz, 1H), 4.94-4.83 (m, 1H), 4.80-4.75 (m, 1H), 4.31 (td, J=3.9, 7.7 Hz, 1H), 3.66 (s, 3H), 3.17 (s, 3H), 1.95 (s, 1H), 1.65 (s, 1H), 1.16 (t, J=7.1 Hz, 1H), 0.86-0.77 (m, 9H), 0.02 (d, J=12.3 Hz, 6H)

LCMS: (M+H⁺): 418.1

TLC (Ethyl acetate:Petroleum ether=1:1) R_{f}=0.26.

### 7. Preparation of Compound 6

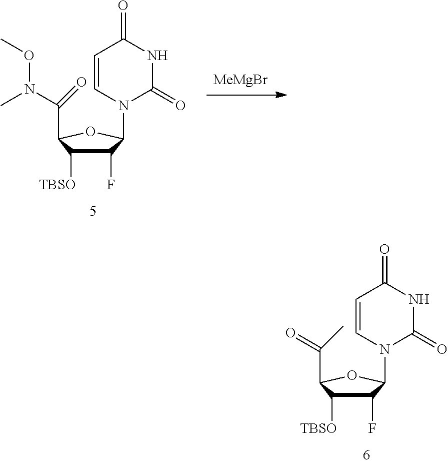

To a solution of compound 5 (52.00 g, 124.55 mmol) in THF (500.00 mL) was added MeMgBr (3 M, 83.03 mL) at −20-0° C. The mixture was stirred at −20° C.-10° C. for 2 hour. TLC indicated compound 5 was consumed and one new spot formed. The reaction mixture was quenched by addition sat. NH₄Cl 500 mL at 0° C., and then diluted with EtOAc (600 mL) and extracted with EtOAc (600 mL*3). Dried over Na₂SO₄, filtered and concentrated under reduced pressure to give a residue. The residue was purified by column chromatography (SiO₂, Petroleum ether/Ethyl acetate=1/0 to 0:1) to get 29 g product and 10 g crude product. Compound 6 (29.00 g, 62.51% yield) was obtained as a white solid.

¹H NMR (400 MHz, CDCl₃) δ=8.90 (br s, 1H), 7.83 (d, J=7.9 Hz, 1H), 5.95-5.77 (m, 2H), 5.10-4.90 (m, 1H), 4.56 (d, J=6.6 Hz, 1H), 4.37 (ddd, J=4.6, 6.6, 15.1 Hz, 1H), 2.27 (s, 3H), 1.04-0.86 (m, 10H), 0.13 (d, J=7.0 Hz, 6H)

LCMS: (M+H⁺): 373.0

TLC (Ethyl acetate:Petroleum ether=1:1) R_{f}=0.4

### 8. Preparation of Compound 7B

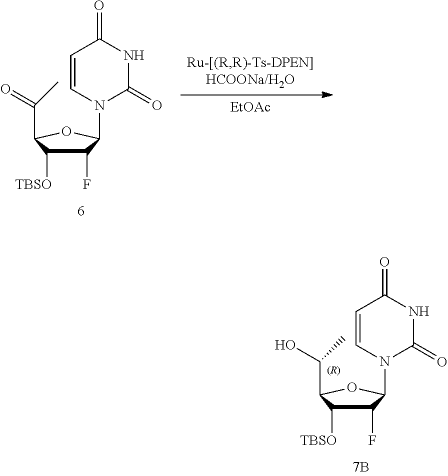

To a solution of compound 6 (24.00 g, 64.44 mmol) in EtOAc (187.50 mL) was added sodium formate (204.65 g, 3.01 mol) in H₂O (750.00 mL) then added [[(1R,2R)-2-amino-1,2-diphenyl-ethyl]-(p-tolylsulfonyl)amino]-chloro-ruthenium; 1-isopropyl-4-methyl-benzene (819.90 mg, 1.29 mmol) in N₂. The mixture was stirred at 25° C. for 20 hour. TLC indicated compound 6 was consumed and one new spot formed. The mixture was extracted with DCM (1000 mL*3). The combined organic was washed with brine (1000 mL), dried over Na₂SO₄, filtered and concentrated to get the crude as a yellow solid. The residue was purified by column chromatography (SiO₂, Petroleum ether/Ethyl acetate=1/0 to 0:1) to get 19.8 g product, then washed with MTBE to get 18 g product. Compound 7B (18.00 g, 74.59% yield) was obtained as a white solid.

¹H NMR (400 MHz, CDCl₃) δ=8.15 (br s, 1H), 7.32 (d, J=7.9 Hz, 1H), 5.63 (d, J=8.2 Hz, 1H), 5.53 (dd, J=5.1, 14.6 Hz, 1H), 5.26-5.04 (m, 1H), 4.41-3.92 (m, 2H), 3.83 (br s, 1H), 2.86 (d, J=2.2 Hz, 1H), 1.13 (d, J=6.6 Hz, 3H), 0.79 (s, 9H), 0.00 (s, 6H)

HPLC: HPLC purity=100%;

SFC: SFC purity=100% ee;

TLC (Petroleum ether:Ethyl acetate=1:1) R_{f}=0.23

### 9. Preparation of Compound 4

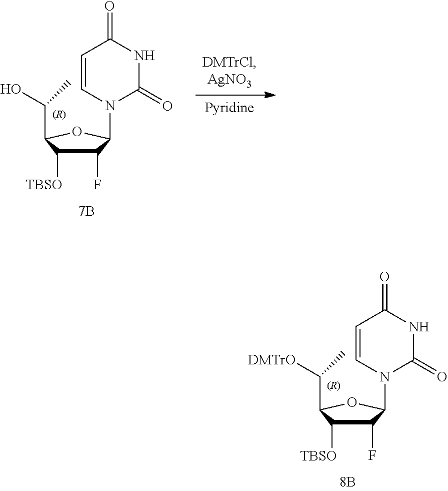

Compound 7B (9.00 g, 24.03 mmol) was dried by azeotropic distillation on a rotary evaporator with pyridine (150 mL) and toluene (150 mL*2).

To a solution of compound 7B (9.00 g, 24.03 mmol) in pyridine (90.00 mL) and THF (270.00 mL) was added DMTCl (15.47 g, 45.66 mmol), then added AgNO₃ (7.02 g, 41.33 mmol, 6.95 mL). The mixture was stirred at 25° C. for 20 hour. TLC indicated compound 7B was consumed and one new spot formed. The mixture was added toluene (200 mL), quenched by addition MeOH (1.3 mL) and stirred for 1 h at 25° C., then filtered through celite, and the Celite plug was washed thoroughly with toluene (150 mL), concentrated under reduced pressure to give a crude. The crude product compound 8B (16.26 g, 100.00% yield) was used into the next step without further purification.

TLC (Petroleum ether:Ethyl acetate=1:1) R_{f}=0.61.

### 10. Preparation of Compound 5′-(R)—C-Me-5′-ODMTr-2′-F-dU

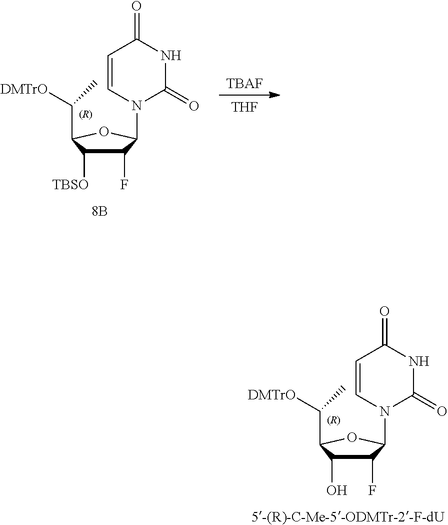

To a solution of compound 8B (32.40 g, 47.87 mmol) in THF (324.00 mL) was added TBAF (1 M, 90.95 mL). The mixture was stirred at 25° C. for 16 hour. TLC indicated compound 8B was consumed and one new spot formed. The reaction mixture was concentrated under reduced pressure to remove solvent. The residue was diluted with EtOAc (300 mL) and washed with sat. NaCl aq. (200 mL*2). Dried over Na₂SO₄, filtered and concentrated under reduced pressure to give a residue. The residue was purified by column chromatography (SiO₂, Petroleum ether/Ethyl acetate=1/0 to 0:1) to get 25 g product. Compound 5′-(R)—C-Me-5′-ODMTr-2′-F-dU (25.00 g, 92.83% yield) was obtained as a yellow solid.

¹H NMR (400 MHz, CDCl₃) δ=7.47 (d, J=7.7 Hz, 2H), 7.38 (dd, J=9.0, 10.1 Hz, 4H), 7.30-7.23 (m, 2H), 7.22-7.18 (m, 1H), 6.83 (br d, J=7.7 Hz, 4H), 5.90 (dd, J=2.4, 17.6 Hz, 1H), 5.31-5.18 (m, 1H), 5.08-4.87 (m, 1H), 4.51 (td, J=5.9, 15.7 Hz, 1H), 3.78 (s, 6H), 3.72-3.60 (m, 2H), 1.05 (d, J=6.4 Hz, 3H).

¹³C NMR (101 MHz, CDCl₃) δ=171.28, 163.37, 158.68, 158.59, 150.04, 146.03, 140.47, 136.06, 130.47, 130.31, 128.08, 127.90, 126.94, 113.23, 113.15, 102.57, 94.11, 92.24, 87.76, 87.43, 87.20, 85.77, 69.46, 69.42, 69.25, 60.45, 55.25, 55.24, 21.06, 17.66, 14.19.

LCMS: (M−H⁺): 561.2

HPLC: HPLC purity=99.05%;

SFC: SFC purity=100% ee;

TLC (Ethyl acetate:Petroleum ether=1:1, R_{f}=0.18)

### Example 30: Synthesis of 5′-(R)—C-Me-5′-ODMTr-2′-F-dU-CNE phosphoramidite

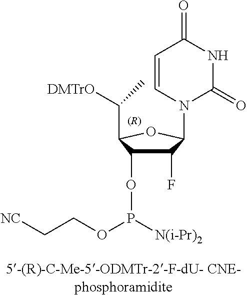

### 1. Preparation of Compound 5′-(R)—C-Me-5′-ODMTr-2′-F-dU-CNE-phosphoramidite

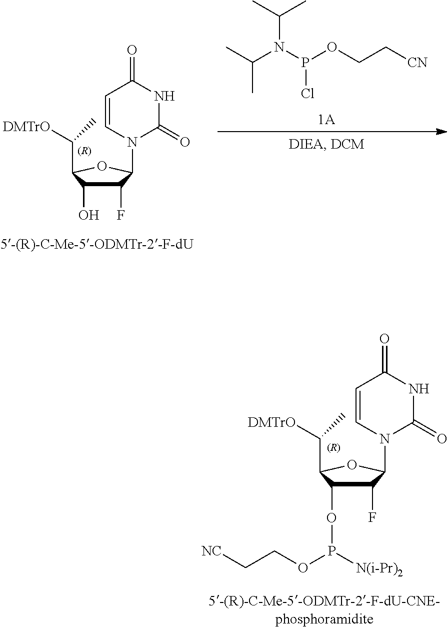

To a solution of 5′-(R)—C-Me-5′-ODMTr-2′-F-dU (4.9 g, 8.71 mmol) in DCM (49 mL) was added DIEA (1.35 g, 10.45 mmol, 1.83 mL) and compound 1A (2.69 g, 9.15 mmol) at 0° C. The mixture was stirred at 0-15° C. for 3 hour. TLC indicated 5′-(R)—C-Me-5′-ODMTr-2′-F-dU was consumed and two new spots formed. The mixture was added sat. NaHCO₃ (20 mL) and extracted with DCM (50 mL*3). The combined organic layers were dried over Na₂SO₄, filtered and concentrated under reduced pressure to give a residue. The residue was purified by MPLC (SiO₂, Ethyl acetate/Petroleum ether=0%, 20%, 40%, 60%, 70%, 100%, 5% TEA) to give 4.3 g (batch 1:3.24 g, batch 2: 1.06 g) of compound 5′-(R)—C-Me-5′-ODMTr-2′-F-dU-CNE-phosphoramidite (4.3 g, 64.72% yield) as a white solid.

### Batch 1:

¹H NMR: (400 MHz, CDCl₃) δ=7.59-7.15 (m, 11H), 6.93-6.76 (m, 4H), 6.01-5.89 (m, 1H), 5.32 (s, 1H), 5.16 (dd, J=8.2, 14.9 Hz, 1H), 5.08-5.02 (m, 1H), 4.93-4.75 (m, 1H), 4.03-3.85 (m, 2H), 3.84-3.77 (m, 6H), 3.74-3.62 (m, 3H), 3.61-3.47 (m, 1H), 2.77 (dt, J=1.9, 6.2 Hz, 1H), 2.72-2.59 (m, 2H), 1.27-1.17 (m, 11H), 0.99 (dd, J=2.0, 6.6 Hz, 3H).

³¹P NMR: (162 MHz, CDCl₃) δ=150.63 (s, 1P), 150.54 (s, 1P), 150.34 (s, 1P), 150.27 (s, 1P), 14.14 (s, 1P).

HPLC: HPLC purity=97.66%;

LCMS: (M−H⁺): 761.3;

TLC (Petroleum ether:Ethyl acetate=3:1), R_{f1}=0.53, R_{f2}=0.62.

### Example 31: Synthesis of 5′-(S)—C-Me-5′-ODMTr-2′-F-dU

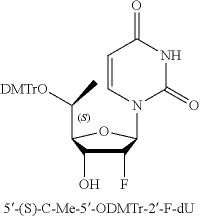

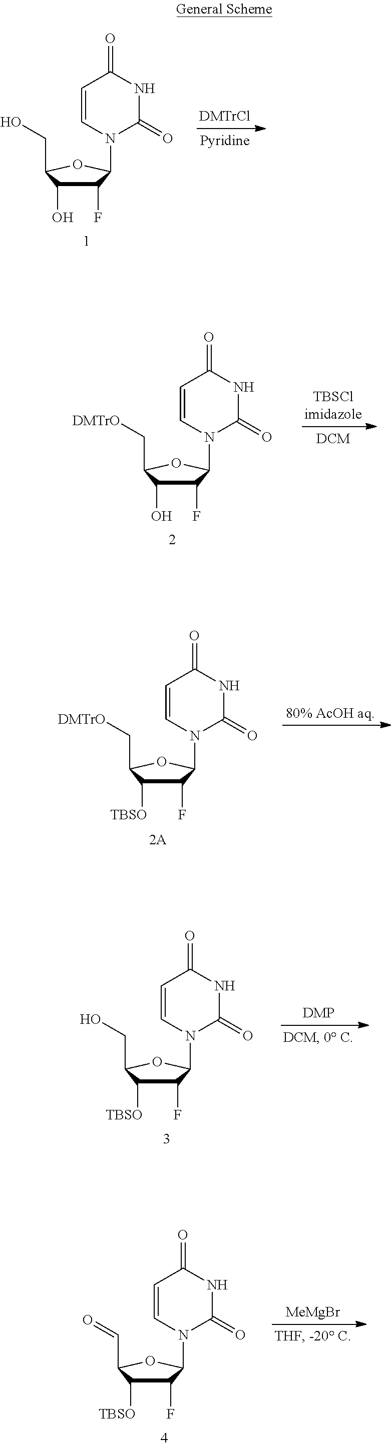

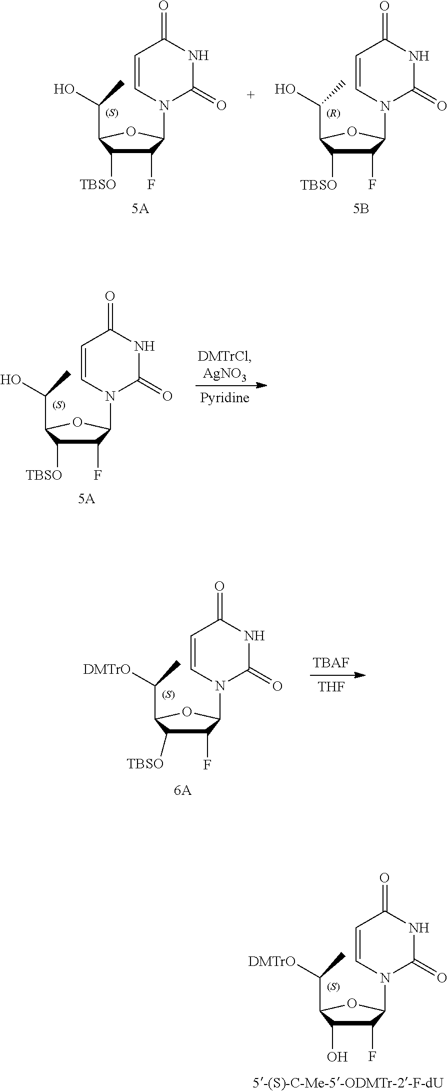

### 1. Preparation of Compound 2

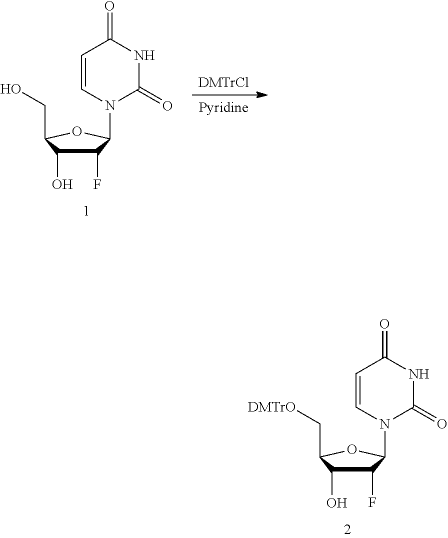

To a solution of compound 1 (100.00 g, 406.19 mmol) in pyridine (550.00 mL) was added DMTCl (165.16 g, 487.43 mmol). The mixture was stirred at 25° C. for 20 hr. TLC indicated compound 1 was consumed and one new spot formed. MeOH (300 mL) was added, the reaction mixture was concentrated under reduced pressure to remove solvent. The residue was dissolved in EtOAc (500 mL) and washed with H₂O (500 mL*3). Dried over Na₂SO₄, filtered and concentrated under reduced pressure to give a residue. The crude product (285.00 g, crude) was yellow solid used into the next step without further purification.

TLC (Ethyl acetate:Petroleum ether=3:1, 5% TEA) R_{f}=0.40

### 2. Preparation of Compound 2A

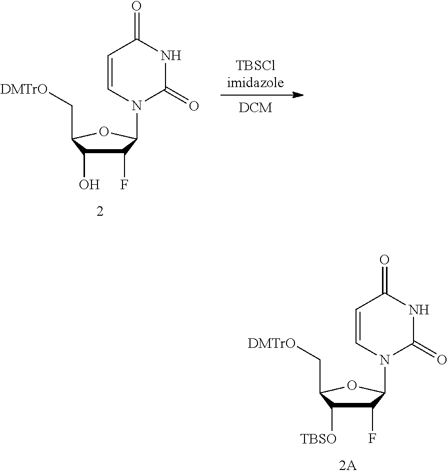

To a solution of compound 2 (222.82 g, 406.19 mmol) in DCM (500.00 mL) was added imidazole (41.48 g, 609.29 mmol) and TBSCl (91.83 g, 609.29 mmol). The mixture was stirred at 25° C. for 20 hour. TLC indicated compound 2 was consumed and one new spot formed. The reaction mixture was concentrated under reduced pressure to remove solvent. The residue was diluted with DCM (500 mL), washed with H₂O (500 mL*3). Dried over Na₂SO₄, filtered and concentrated under reduced pressure to give a residue. The crude product (330.00 g, crude) was white solid used into the next step without further purification.

TLC (Ethyl acetate:Petroleum ether=3:1) R_{f}=0.65

### 3. Preparation of Compound 3

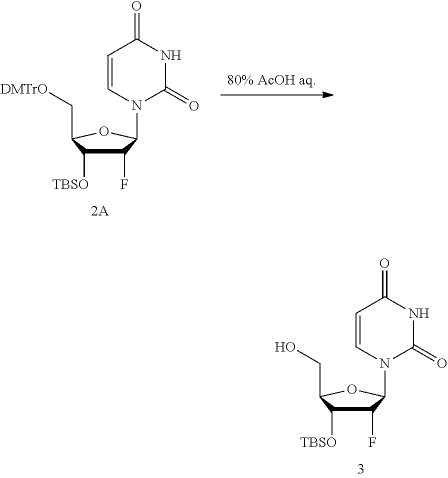

A solution of compound 2A (269.23 g, 406.19 mmol) in AcOH (400.00 mL) 80% aq. was stirred at 25° C. for 15 hour. TLC indicated compound 2A was remained a little and one new spot formed. The reaction mixture was quenched by sat. NaHCO₃ aq. until pH>7 at 25° C., and then diluted with EtOAc (500 mL) and extracted with EtOAc (500 mL*3). Dried over Na₂SO₄, filtered and concentrated under reduced pressure to give a residue. The residue was purified by column chromatography (SiO₂, Petroleum ether/Ethyl acetate=1/0 to 0/1 10% DCM). Got 60 g product and recovered 130 compound 2A. Compound 3 (60.00 g, 40.98% yield) was obtained as a white solid.

TLC (Ethyl acetate:Petroleum ether=3:1) R_{f}=0.45

### 4. Preparation of Compound 4

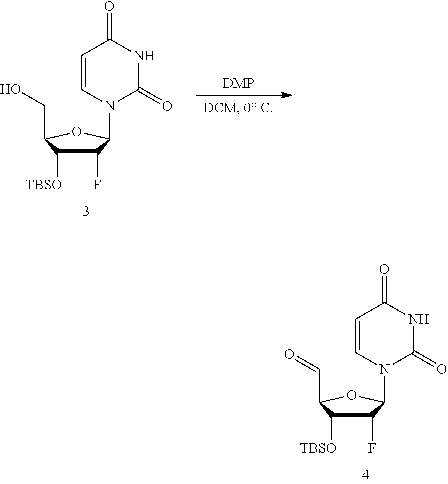

To a solution of compound 3 (10.00 g, 27.74 mmol) in DCM (400.00 mL) was added DMP (14.12 g, 33.29 mmol) at 0° C. The mixture was stirred at 0-50° C. for 6 hour. TLC indicated compound 3 was consumed and one new spot formed. The reaction mixture was quenched by addition sat. Na₂S₂O₃ aq. (300 mL) and sat. NaHCO₃ aq. (300 mL) at 0° C., and then diluted with EtOAc (800 mL) and extracted with EtOAc (800 mL*3). Dried over Na₂SO₄, filtered and concentrated under reduced pressure at 25° C. The crude product compound 4 (9.50 g, crude as yellow solid) was used into the next step without further purification.

TLC (Ethyl acetate:Petroleum ether=3:1) R_{f}=0.37

### 3. Preparation of Compound 5

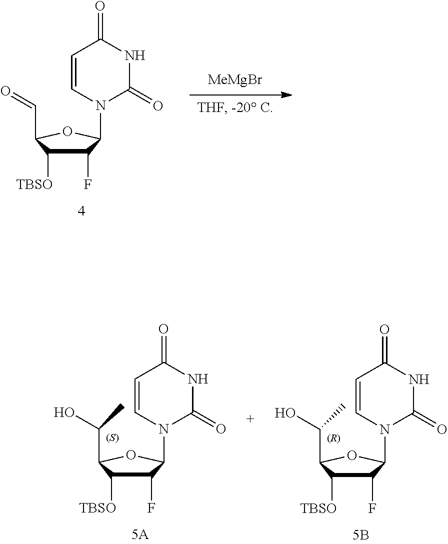

To a solution of MeMgBr (3 M, 35.33 mL) in THF (200 mL) was added compound 4 (9.50 g, 26.50 mmol) in THF (300 mL) at −25° C. under N₂. The mixture was stirred at −25° C.-25° C. for 1 hour. TLC indicated compound 4 was consumed and two new spots formed. The reaction mixture was quenched by addition NH₄Cl (300 mL) at 0° C., and then diluted with EtOAc (400 mL) and extracted with EtOAc (400 mL*3). Dried over Na₂SO₄, filtered and concentrated under reduced pressure to give a residue. The residue was purified by column chromatography (SiO₂, Petroleum ether/Ethyl acetate=1/0 to 0/1) to get 1 g Compound 5A, 0.6 g Compound 5B, other mixture of Compound 5A and Compound 5B. Compound 5A (1.00 g, 10.08% yield) was obtained as a white solid. Compound 5B (600.00 mg, 6.04% yield) was obtained as a white solid..

### Compound 5A:

¹H NMR (400 MHz, DMSO-d₆) δ=7.89 (d, J=8.2 Hz, 1H), 5.82 (dd, J=2.2, 16.9 Hz, 1H), 5.53 (d, J=8.1 Hz, 1H), 5.09 (d, J=4.6 Hz, 1H), 5.05-4.87 (m, 1H), 4.22 (ddd, J=4.5, 6.7, 18.1 Hz, 1H), 3.73-3.65 (m, 1H), 3.62 (br d, J=6.7 Hz, 1H), 1.14-1.05 (m, 3H), 0.81-0.67 (m, 9H), 0.00 (d, J=2.3 Hz, 6H);

LCMS: (M+H⁺): 375.1; LCMS purity=90.1%;

HPLC: purity 97.9%;

TLC (Ethyl acetate:Petroleum ether=1:1) 5A: R_{f1}=0.42; 5B: R_{f2}=0.47.

### Compound 5B:

¹H NMR (400 MHz, DMSO-d₆) δ=7.78 (d, J=8.1 Hz, 1H), 5.84 (dd, J=4.0, 15.7 Hz, 1H), 5.60-5.49 (m, 1H), 5.14-5.03 (m, 1H), 4.98-4.89 (m, 1H), 4.32 (td, J=4.9, 12.0 Hz, 1H), 3.86-3.73 (m, 1H), 3.70-3.57 (m, 1H), 1.00 (d, J=6.6 Hz, 3H), 0.85-0.67 (m, 9H), 0.06-−0.10 (m, 6H);

LCMS: (M+H⁺): 375.1;

HPLC: purity 75.9%;

TLC (Ethyl acetate:Petroleum ether=1:1) 5A: R_{f1}=0.42; 5B: R_{f2}=0.47.

### 6. Preparation of Compound 6A

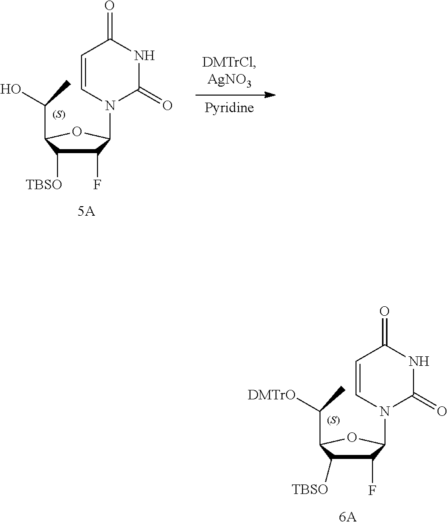

Compound 5A (1.00 g, 2.67 mmol) was dried by azeotropic distillation on a rotary evaporator with pyridine (20 mL) and toluene (20 mL*2). To a solution of 5A (1.00 g, 2.67 mmol) in THF (30.00 mL) and pyridine (9.93 g, 125.49 mmol, 10.13 mL) was added 1-[chloro-(4-methoxyphenyl)-phenyl-methyl]-4-methoxy-benzene (1.72 g, 5.07 mmol) then added AgNO₃ (780.11 mg, 4.59 mmol) under N₂. The mixture was stirred at 25° C. for 20 hour. TLC indicated compound 5A was consumed and one new spot formed. The mixture was added toluene (30 mL), quenched by addition MeOH (0.1 mL) and stirred for 1 h at 25° C., then filtered through celite, and the celite plug was washed thoroughly with toluene (20 mL), concentrated under reduced pressure to give a crude. The residue was purified by column chromatography (SiO₂, Petroleum ether/Ethyl acetate=1/0 to 0/1) to get 1.1 g product. Compound 6A (1.10 g, 60.87% yield) was obtained as a yellow solid.

¹H NMR (400 MHz, CDCl₃) δ=8.08 (d, J=8.2 Hz, 1H), 7.48 (br d, J=7.5 Hz, 2H), 7.43-7.27 (m, 9H), 6.93 (dd, J=4.0, 8.6 Hz, 4H), 6.15 (dd, J=3.1, 14.1 Hz, 1H), 5.68 (d, J=8.2 Hz, 1H), 5.09-4.87 (m, 1H), 4.34 (td, J=5.2, 14.5 Hz, 1H), 4.01 (br d, J=4.9 Hz, 1H), 3.91 (d, J=1.5 Hz, 7H), 3.83 (br dd, J=2.8, 6.7 Hz, 1H), 2.29 (s, 1H), 2.19-2.03 (m, 1H), 1.10 (d, J=6.4 Hz, 3H), 1.04-1.00 (m, 1H), 0.92 (s, 9H), 0.18 (s, 1H), 0.15 (s, 3H), 0.00 (s, 3H).

TLC (Petroleum ether:Ethyl acetate=1:1) R_{f}=0.64

### 7. Preparation of 5′-(S)—C-Me-5′-ODMTr-2′-F-dU

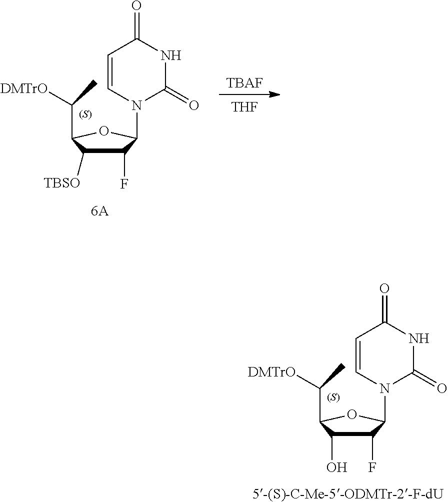

To a solution of compound 6A (1.00 g, 1.48 mmol) in THF (15.00 mL) was added TBAF (733.96 mg, 2.81 mmol). The mixture was stirred at 25° C. for 3 hour. TLC indicated compound 6A was consumed and one new spot formed. The mixture was concentrated under reduced pressure to give a residue. The residue was dissolved by EtOAc (20 mL) and washed by NaCl (5%, aq. 20 mL), extracted with EtOAc (20 mL*3). Dried over Na₂SO₄, filtered and concentrated under reduced pressure to give a residue. The residue was purified by column chromatography (SiO₂, Petroleum ether/Ethyl acetate=1/0 to 0/1) to get 0.7 g product. Compound 5′-(S)—C-Me-5′-ODMTr-2′-F-dU (700.00 mg, 84.07% yield) was obtained as a yellow solid.

¹H NMR (400 MHz CDCl₃,) δ=7.75 (d, J=8.2 Hz, 1H), 7.51-7.46 (m, 2H), 7.44-7.36 (m, 4H), 7.36-7.32 (m, 1H), 7.29-7.24 (m, 1H), 6.88 (dd, J=2.2, 8.9 Hz, 4H), 5.92 (dd, J=1.5, 18.0 Hz, 1H), 5.34 (s, 1H), 5.19-4.95 (m, 1H), 3.84 (d, J=1.1 Hz, 6H), 3.80-3.71 (m, 1H), 1.12 (d, J=6.5 Hz, 3H);

¹³C NMR (101 MHz, CDCl₃) δ=162.95, 158.70, 149.74, 145.60, 140.44, 136.26, 136.06, 130.71, 128.54, 127.98, 127.55, 126.79, 113.81, 113.19, 112.99, 112.34, 102.72, 102.47, 88.87, 88.60, 88.53, 88.26, 87.22, 85.76, 85.52, 69.12, 68.93, 68.52, 68.28, 55.61, 55.37, 54.88, 18.29;

LCMS: (M−H⁺): 561.2;

HPLC: purity 93.2%;

TLC (Ethyl acetate:Petroleum ether=1:1) R_{f}=0.17.

### 8. Preparation of Compound 5′-(S)—C-Me-5′-ODMTr-2′-F-dU-CNE

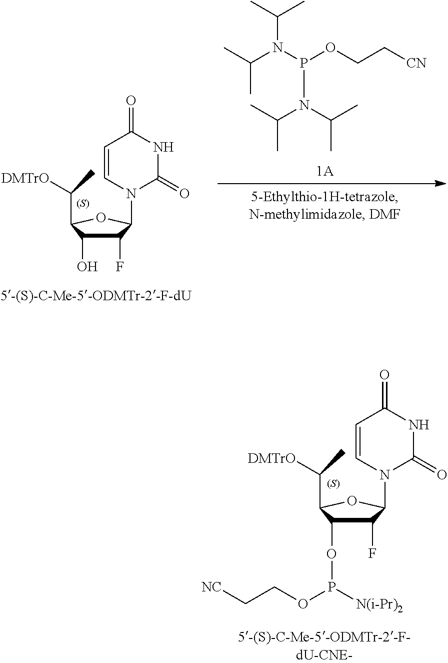

Compound 5′-(S)—C-Me-5′-ODMTr-2′-F-dU (4.85 g, 8.62 mmol) was dried by azeotropic distillation on a rotary evaporator with toluene (10 mL*3). To a solution of compound 5′-(S)—C-Me-5′-ODMTr-2′-F-dU (4.85 g, 8.62 mmol) in DMF (48.5 mL) was added N— methylimidazole (1.42 g, 17.24 mmol, 1.37 mL) and 5-ethylsulfanyl-2H-tetrazole (1.12 g, 8.62 mmol), degassed and purged with N₂ for 3 times. Then added 3-bis (diisopropylamino)phosphanyloxypropanenitrile (3.90 g, 12.93 mmol, 4.11 mL). The mixture was stirred at 15° C. for 2 hr in N₂. TLC indicated compound 5′-(S)—C-Me-5′-ODMTr-2′-F-dU was consumed and two new spot formed. The mixture was added sat. NaHCO₃ (aq., 50 mL), extracted with Ethyl acetate (50 mL*3). The combined organic layers were washed with H₂O (50 mL*2), dried over Na₂SO₄, filtered and concentrated under reduced pressure to give a residue at 30° C. waterbath under N₂ atmosphere. The residue was purified by column chromatography (SiO₂, Petroleum ether/Ethyl acetate=I/O to 0:1) get 4 g product. Compound 5′-(S)—C-Me-5′-ODMTr-2′-F-dU-CNE (4 g, 56.66% yield) was obtained as a white solid.

¹H NMR (400 MHz, CDCl₃) δ=8.78 (br s, 1H), 8.00-7.79 (m, 1H), 7.41-7.08 (m, 10H), 6.86-6.64 (m, 4H), 5.93 (br t, J=17.2 Hz, 1H), 5.46 (dd, J=2.8, 8.1 Hz, 1H), 5.19-4.93 (m, 1H), 4.51-4.25 (m, 1H), 3.99-3.87 (m, 1H), 3.84-3.71 (m, 6H), 3.70-3.61 (m, 2H), 3.59-3.30 (m, 4H), 2.94-2.77 (m, 2H), 2.73-2.62 (m, 1H), 2.56-2.41 (m, 1H), 2.23 (t, J=6.2 Hz, 1H), 1.32-0.82 (m, 22H);

¹³C NMR (101 MHz, CDCl₃) δ=163.21, 163.07, 158.72, 158.65, 150.08, 149.95, 145.98, 139.98, 136.48, 136.25, 136.16, 130.76, 130.71, 128.60, 127.69, 127.05, 126.95, 117.76, 113.00, 102.41, 88.32, 87.08, 86.45, 69.83, 69.10, 68.62, 60.39, 58.26, 58.22, 58.16, 58.07, 57.88, 55.26, 55.21, 45.36, 45.30, 36.47, 31.44, 24.51, 22.94, 21.04, 20.28, 18.67, 14.20;

³¹P NMR (162 MHz, CHLOROFORM-d) δ=150.70 (s, 1P), 150.65 (s, 1P), 150.63 (s, 1P), 150.54 (s, 1P), 14.18 (s, 1P);

LCMS: (M−H⁺): 761.2;

HPLC: HPLC purity=52.15%+41.00%;

TLC: (Ethyl acetate:Petroleum ether=3:1), R_{f1}=0.32, R_{f2}=0.4.

### Example 32: Synthesis of 5′-(R)—C-Me-5′-ODMTr-2′-OMe-U

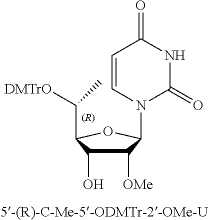

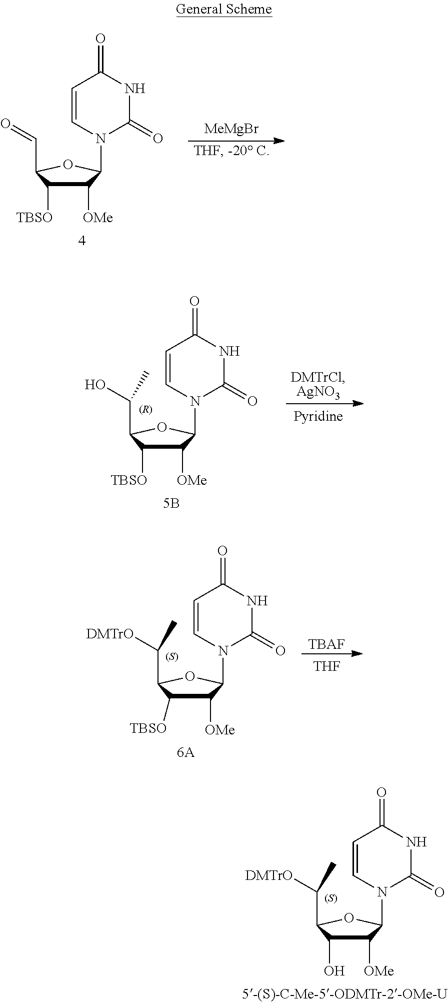

### 1. Preparation of Compound 5B

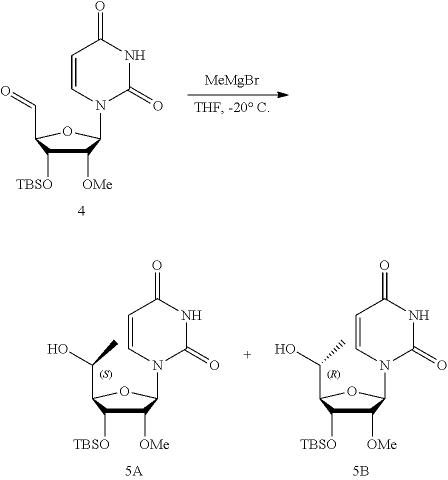

To a solution of compound 4 (19.00 g, 51.29 mmol) in THF (140 mL) was dropwise in MeMgBr (3 M, 68.39 mL) (a solution in 140 mL THF) at −20° C. over 10 min. The mixture was stirred at −20° C.-20° C. for 30 min. TLC showed compound 4 was partly remained and new spot was detected. Then the mixture was stirred at 20° C. for 20 min. TLC and LCMS showed compound 4 was partly remained and new spot was detected. The reaction mixture was quenched by addition sat. NH₄Cl (200 mL) at 0° C., and then diluted with EtOAc (500 mL) and extracted with EtOAc (500 mL*3). The combined organic layers were dried over anhydrous Na₂SO₄, filtered and concentrated under reduced pressure to give a residue. The residue was purified by MPLC (flash Silica (CS), 40-60 μm, 60A, 220 g, Ethyl acetate/Petroleum ether=0%, 10%, 20%, 30%, 50%, 60%, 70%, 80%, 100%) to give compound 5A (1.80 g, 9.08% yield) as a white solid. Compound 5B (1.60 g, 8.07% yield) was obtained as a white solid.

TLC (plate 1: Petroleum ether:Ethyl acetate=1:3) R_{f1}=0.39, R_{f2}=0.32

### Compound 5A

¹H NMR (400 MHz, DMSO-d₆) δ=11.26 (s, 1H), 7.98 (d, J=8.2 Hz, 1H), 5.75 (d, J=4.6 Hz, 1H), 5.58 (d, J=8.2 Hz, 1H), 5.10 (d, J=4.4 Hz, 1H), 4.19 (t, J=4.6 Hz, 1H), 3.72 (br t, J=4.9 Hz, 2H), 3.60 (br d, J=2.9 Hz, 1H), 3.25 (s, 3H), 1.07 (d, J=6.4 Hz, 3H), 0.79 (s, 9H), 0.00 (s, 6H)

LCMS (M+H⁺): 387.1

TLC (plate 1: Petroleum ether:Ethyl acetate=1:3) R_{f1}=0.39

### Compound 5B

¹H NMR (400 MHz, DMSO-d₆) δ=11.28 (s, 1H), 7.80 (d, J=8.2 Hz, 1H), 5.77 (d, J=6.8 Hz, 1H), 5.58 (d, J=8.2 Hz, 1H), 5.07 (br s, 1H), 4.33-4.29 (m, 1H), 3.77 (dd, J=5.1, 6.6 Hz, 1H), 3.72-3.64 (m, 1H), 3.55 (dd, J=2.0, 4.0 Hz, 1H), 3.18 (s, 3H), 1.01 (d, J=6.6 Hz, 3H), 0.78 (s, 9H), 0.00 (s, 6H)

LCMS (M+H⁺): 387.2

TLC (Petroleum ether:Ethyl acetate=1:2) R_{f2}=0.32

### 2. Preparation of Compound 6B

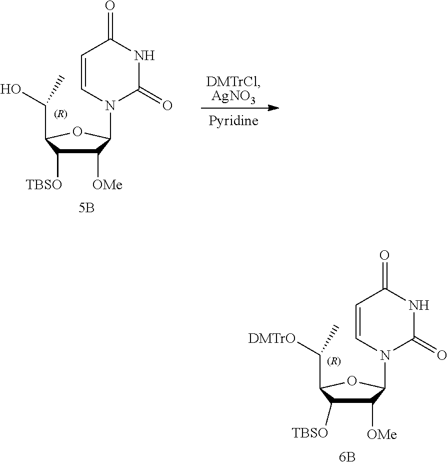

Compound 5B (1.10 g, 2.85 mmol) was dried by azeotropic distillation on a rotary evaporator with Pyridine (20 mL) and toluene (20 mL*2). To a solution of compound 5B (1.10 g, 2.85 mmol) in THF (33.00 mL) and pyridine (11.52 g, 145.70 mmol, 11.76 mL) was added DMTCl (1.83 g, 5.41 mmol), then added AgNO₃ (831.53 mg, 4.90 mmol, 823.30 uL). The mixture was stirred at 25° C. for 20 hours. TLC showed compound 5B was consumed and new spot was detected. The mixture was added toluene (30 mL), quenched by addition MeOH (0.1 mL) and stirred for 1 h at 25° C., then filtered through celite, and the celite plug was washed thoroughly with toluene (20 mL), concentrated under reduced pressure to give a crude. Compound 6B (3.00 g, crude) was obtained as a yellow oil.

TLC (Petroleum ether:Ethyl acetate=1:1) Rf=0.43.

### 3. Preparation of Compound 5′-(R)—C-Me-5′-ODMTr-2′-OMe-U

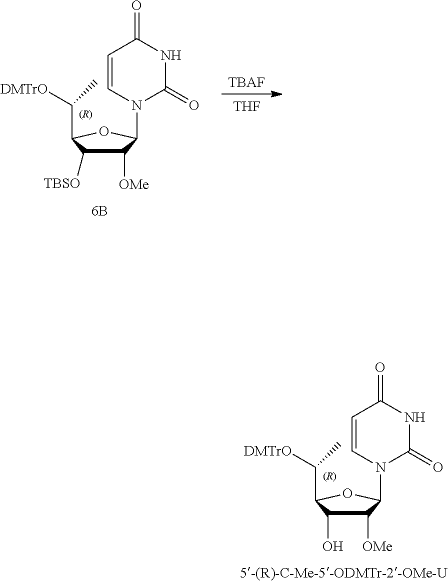

To a solution of compound 6B (1.96 g, 2.85 mmol) in THF (40.00 mL) was added TBAF (1 M, 5.41 mL). The mixture was stirred at 25° C. for 3 hour. TLC showed compound 6B was consumed and one new spot was detected. The mixture was concentrated under reduced pressure to give a residue. The residue was dissolved by EtOAc (50 mL) and washed by NaCl (5%, aq. 50 mL), extracted with EtOAc (50 mL*3), dried over Na₂SO₄, filtered and concentrated under reduced pressure to give a residue. The residue was purified by MPLC (SiO₂, silica gel was washed by Petroleum ether (5% TEA),Ethyl acetate/Petroleum ether=0%; 20%; 50%; 70%, 80% to 100%) to give compound 5′-(R)—C-Me-5′-ODMTr-2′-OMe-U (950.00 mg, 56.95% yield) was obtained as a white solid.

¹H NMR (400 MHz, DMSO-d₆) δ=11.37 (s, 1H), 7.44 (d, J=7.6 Hz, 2H), 7.36-7.19 (m, 8H), 6.90 (d, J=8.9 Hz, 4H), 5.78-5.71 (m, 1H), 5.21-5.13 (m, 2H), 4.30 (q, J=5.6 Hz, 1H), 3.78-3.64 (m, 8H), 3.52-3.42 (m, 1H), 3.34 (s, 3H), 0.79 (d, J=6.4 Hz, 3H)

¹³C NMR (101 MHz, DMSO-d₆) δ=163.24, 158.58, 158.55, 150.82, 146.71, 140.99, 136.59, 136.48, 130.63, 128.33, 128.16, 127.07, 113.58, 113.52, 102.25, 87.59, 86.52, 86.29, 82.06, 69.89, 68.16, 58.10, 55.51, 55.49, 33.72, 23.07, 17.61, 15.20

HPLC purity: 98.168%

LCMS (M+H⁺): 573.1

TLC (Petroleum ether:Ethyl acetate=1:2) R_{f}=0.10

### 4. Preparation of Compound 5′-(R)—C-Me-5′-ODMTr-2′-OMe-U-CNE-phosphoramidite

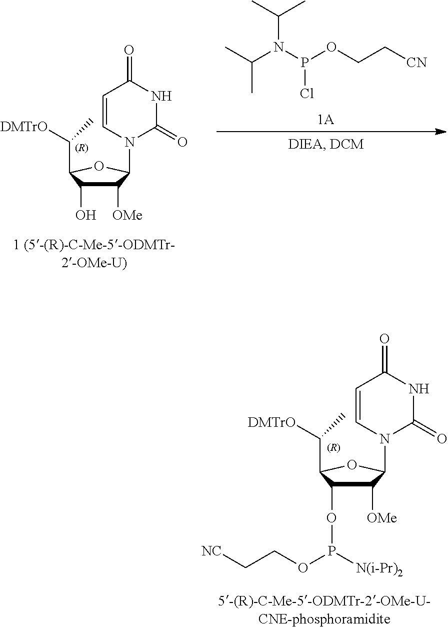

DIEA (1.32 g, 10.23 mmol, 1.79 mL) were added consecutively to a stirred solution of compound 1 (4.9 g, 8.53 mmol) in anhyd. DCM (50 mL) under Ar atm., and then added compound 1A (43.25 mg, 182.73 umol) at 0° C. After stirring at 0° C.-15° C. for 3 hr. LCMS showed compound 1 was partly remained and two major spots was detected. Then added compound 1A (201.82 mg, 852.74 umol). After stirring at 0° C.-15° C. for 1 hr, TLC showed compound 1 was partly remained and two major spots was detected. The mixture was added sat. NaHCO₃ (aq., 20 mL) and extracted with DCM (50 mL*3). The combined organic layers were dried over Na₂SO₄, filtered and concentrated under reduced pressure to give a residue. The residue was purified by MPLC (SiO₂, Ethyl acetate/Petroleum ether=0%, 20%, 40%, 60%, 70%, 100%, 5% TEA) to give compound 5′-(R)—C-Me-5′-ODMTr-2′-OMe-U-CNE-phosphoramidite (4.0 g, 60.54% yield) was obtained as a white solid.

¹H NMR (400 MHz, CDCl₃) δ=7.56-7.48 (m, 2H), 7.47-7.36 (m, 4H), 7.33-7.20 (m, 5H), 6.86 (td, J=2.5, 8.9 Hz, 4H), 5.94 (t, J=4.7 Hz, 1H), 5.06 (dd, J=1.2, 8.1 Hz, 1H), 4.91-4.71 (m, 1H), 4.04-3.86 (m, 4H), 3.82 (s, 6H), 3.76-3.65 (m, 3H), 3.53 (d, J=8.7 Hz, 4H), 2.71-2.53 (m, 3H), 1.27-1.22 (m, 10H), 1.01 (t, J=6.3 Hz, 3H)

³¹P NMR (162 MHz, CDCl₃) δ=150.16, 149.61, 14.16

LCMS: (M−H⁺): 773.3

HPLC purity: 40.8%+50.0%

TLC (Petroleum ether:Ethyl acetate=1:3, 5% TEA) R_{f1}=0.60, R_{f2}=0.55

### Example 33: Synthesis of 5′-(S)—C-Me-5′-ODMTr-2′-OMe-U

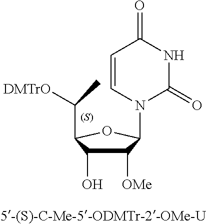

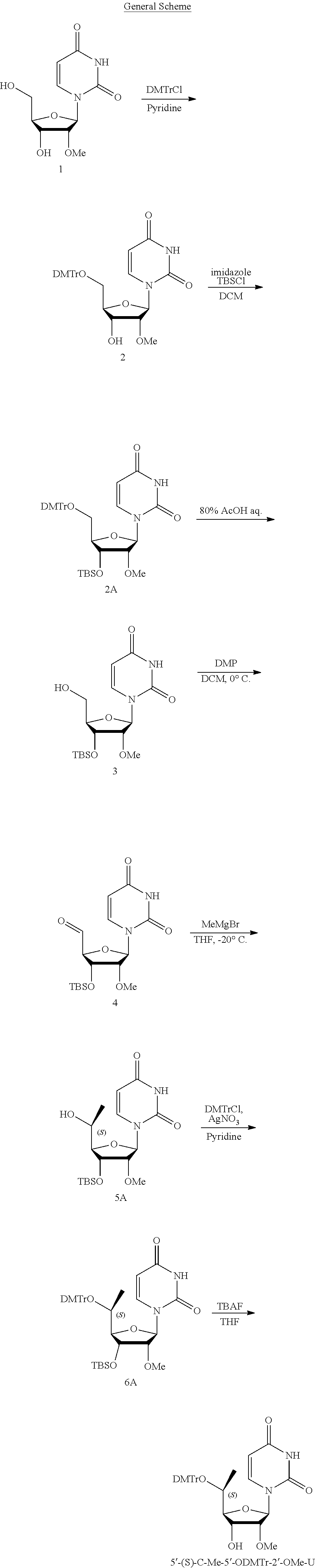

### 1. Preparation of Compound 2

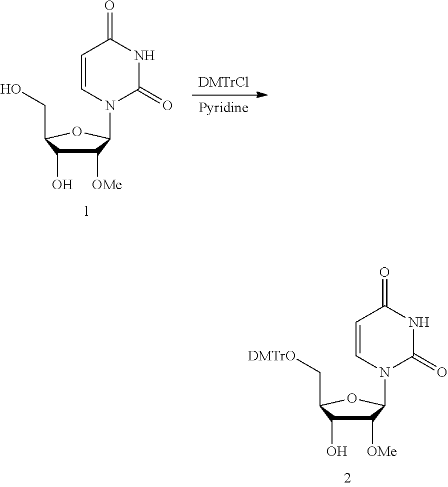

To a solution of compound 1 (10.00 g, 38.73 mmol) in pyridine (80.00 mL) was added DMTCl (15.75 g, 46.48 mmol) at 0° C. The mixture was stirred at 0-20° C. for 16 hours. TLC showed the starting material was consumed and one new spot was detected. The resulting mixture was concentrated under reduced pressure to give a residue. The residue was dissolved by addition ethyl acetate (300 mL) and H₂O (150 mL), and extracted with ethyl acetate (300 mL*3). The combined organic layers were dried over Na₂SO₄, filtered and concentrated under reduced pressure to give a crude (25 g) as a yellow solid. Compound 2 (25.00 g, crude) was obtained as a yellow oil. TLC (Petroleum ether:Ethyl acetate=1:3, 5% TEA) R_{f}=0.1.

### 2. Preparation of Compound 2A

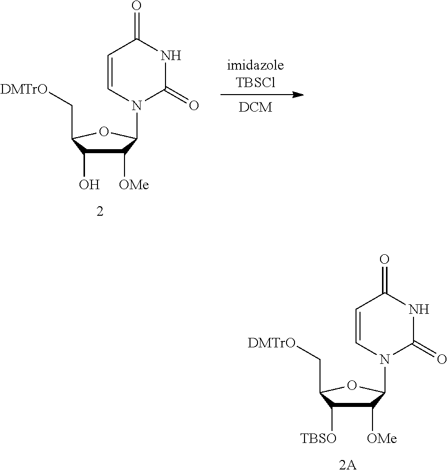

To a solution of compound 2 (24.00 g, 42.81 mmol) in DCM (200.00 mL) was added imidazole (5.83 g, 85.62 mmol) and TBSCl (9.68 g, 64.21 mmol). The mixture was stirred at 20° C. for 14 hours. TLC showed compound 2 was partly remained and one major spot was detected. The resulting solution was combined with another batch product (1 g scale) and diluted with DCM (300 mL), washed with NaHCO₃ (aq., 100 mL) and brine (100 mL). The organic layer was dried over anhydrous Na₂SO₄, filtered and concentrated under reduced pressure to give 28 g crude. Compound 2A (26.88 g, 93.04% yield) (Yield From Conversion Rate) was obtained as a white solid.

¹H NMR (400 MHz, CDCl₃) δ=9.29 (br s, 1H), 8.64 (br d, J=4.2 Hz, 1H), 8.19 (d, J=8.2 Hz, 1H), 7.40-7.25 (m, 12H), 7.20 (d, J=8.8 Hz, 2H), 6.89-6.81 (m, 5H), 5.98 (s, 1H), 5.28 (d, J=7.9 Hz, 1H), 4.43 (dd, J=5.0, 8.0 Hz, 1H), 4.11 (br d, J=8.2 Hz, 1H), 3.84-3.78 (m, 8H), 3.73-3.55 (m, 5H), 3.42-3.36 (m, 1H), 1.00-0.83 (m, 16H), 0.15-0.04 (m, 7H);

TLC (Petroleum ether:Ethyl acetate=1:3) Rf=0.47.

### 3. Preparation of Compound 3

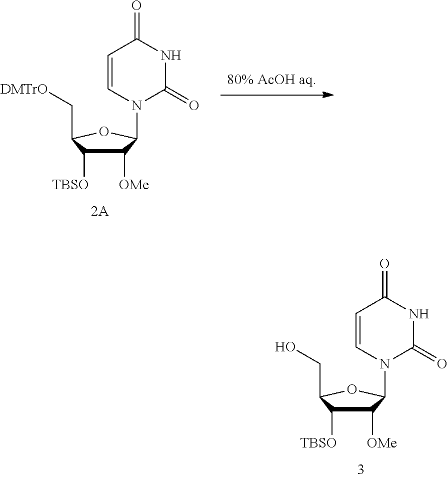

To a solution of compound 2A (27.00 g, 40.01 mmol) in CH₃COOH/H₂O (V/V=80%, 100 mL). The mixture was stirred at 20° C. for 16 hours. TLC showed compound 2A was consumed and one major spot was detected. The resulting solution was diluted with Ethyl acetate (300 mL) and added sat. NaHCO₃ (aq.) to pH-7, then extracted with Ethyl acetate (300 mL*3). The organic layer was dried over anhydrous Na₂SO₄, filtered and concentrated under reduced pressure to give a crude. The crude was purified by MPLC (SiO₂, Petroleum ether:Ethyl acetate=5:1 to 1:3). Compound 3 (10.00 g, 67.10% yield) was obtained as a white solid.

¹H NMR (400 MHz,) CDCl₃ 6=8.18 (br s, 1H), 7.56 (d, J=8.2 Hz, 1H), 5.62 (dd, J=2.1, 8.3 Hz, 1H), 5.55 (d, J=4.2 Hz, 1H), 4.25 (t, J=5.1 Hz, 1H), 3.91-3.86 (m, 2H), 3.65 (br dd, J=7.1, 11.9 Hz, 1H), 3.38 (s, 3H), 2.46 (br d, J=4.2 Hz, 1H), 0.81 (s, 9H), 0.01 (d, J=5.5 Hz, 6H);

TLC (Petroleum ether:Ethyl acetate=1:1) Rf=0.28.

### 4. Preparation of Compound 4

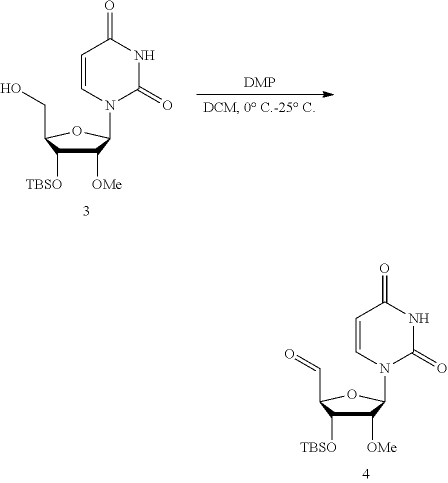

To a solution of compound 3 (3.00 g, 8.05 mmol) in DCM (50.00 mL) was added DMP (4.10 g, 9.66 mmol) at 0° C. The mixture was stirred at 25° C. for 1.5 h. TLC showed compound 3 was partly remained and one major spot was detected. The mixture was diluted and added EtOAc (50 mL) and quenched by addition Na₂S₂O₃ (5% aq., 80 mL) and sat. NaHCO₃ (aq., 80 mL) at 0° C. and stirred for 20 min, extracted with EtOAc (200 mL*3). The combined organic layers were dried over Na₂SO₄, filtered, and concentrated under reduced pressure at 25° C. water bath to give a crude. Compound 4 (2.50 g, crude) was obtained as a white solid.

¹H NMR (400 MHz, CDCl₃) δ=9.64 (s, 1H), 7.55-7.44 (m, 1H), 5.73-5.56 (m, 2H), 4.42-4.25 (m, 1H), 3.80 (br t, J=4.5 Hz, 1H), 3.42-3.20 (m, 3H), 0.78 (s, 9H), 0.07-0.14 (m, 6H);

TLC (Petroleum ether:Ethyl acetate=1:3) R_{f}=0.18.

### 5. Preparation of Compound 5A and 5B

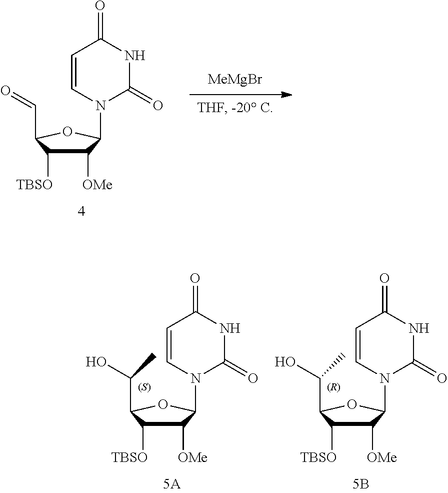

To a solution of compound 4 (2.50 g, 6.75 mmol) in THF (30 mL) was added dropwise MeMgBr (3 M, 9.00 mL) (a solution in 30 mL THF) at −20° C. over 10 min. The mixture was stirred at −20° C.-20° C. for 30 min. TLC showed compound 4 was partly remained and new spot was detected. Then the mixture was stirred at 20° C. for 20 min. TLC showed compound 4 was partly remained and new spot was detected. The residue was purified by MPLC (SiO₂, Petroleum ether:Ethyl acetate=5:1, 3:1, 1:1,to 1:2) to compound 5A (320.00 mg, 12.27% yield) (Yield From Conversion Rate) was obtained as a white solid and compound 5B (480.00 mg, 18.37% yield) (Yield From Conversion Rate) was obtained as a white solid.

TLC (Petroleum ether:Ethyl acetate=1:2) R_{f1}=0.39, R_{f2}=0.32

### Compound 5A:

¹H NMR (400 MHz, DMSO-d₆) δ=11.26 (s, 1H), 7.98 (d, J=8.2 Hz, 1H), 5.75 (d, J=4.6 Hz, 1H), 5.58 (d, J=8.2 Hz, 1H), 5.10 (d, J=4.4 Hz, 1H), 4.19 (t, J=4.6 Hz, 1H), 3.72 (br t, J=4.9 Hz, 2H), 3.60 (br d, J=2.9 Hz, 1H), 3.25 (s, 3H), 1.07 (d, J=6.4 Hz, 3H), 0.79 (s, 9H), 0.00 (s, 6H)

TLC (Petroleum ether:Ethyl acetate=1:2) R_{f1}=0.39

### Compound 5B:

¹H NMR (400 MHz, DMSO-d₆) δ=11.28 (s, 1H), 7.80 (d, J=8.2 Hz, 1H), 5.77 (d, J=6.8 Hz, 1H), 5.58 (d, J=8.2 Hz, 1H), 5.07 (br s, 1H), 4.33-4.29 (m, 1H), 3.77 (dd, J=5.1, 6.6 Hz, 1H), 3.72-3.64 (m, 1H), 3.55 (dd, J=2.0, 4.0 Hz, 1H), 3.18 (s, 3H), 1.01 (d, J=6.6 Hz, 3H), 0.78 (s, 9H), 0.00 (s, 6H)

TLC (Petroleum ether:Ethyl acetate=1:2) R_{f2}=0.32

### 6. Preparation of Compound 6A

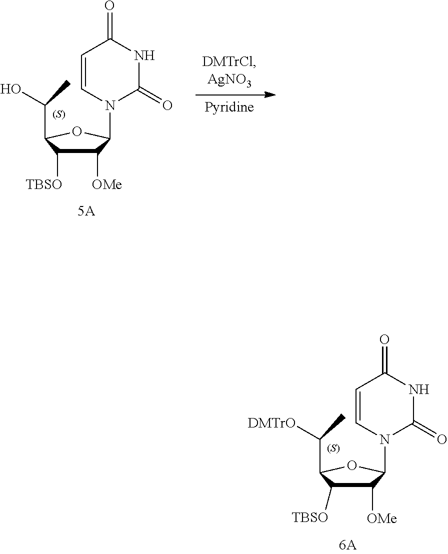

To a mixture of pre-purified compound 5A (740.00 mg, 1.91 mmol), DMTCl (1.23 g, 3.63 mmol), and pyridine (7.10 g, 89.75 mmol, 7.24 mL) in anhyd. THF (30.00 mL) was added AgNO₃ (558.06 mg, 3.29 mmol). The mixture was stirred at 25° C. under N₂ for 16 h. TLC showed compound 5A was consumed and one new spot was detected. The mixture was quenched by addition of MeOH (0.1 mL) and diluted with toluene (30 mL). After stirred for an additional 1 h, the mixture was filtered through Celite, and the Celite plug was washed thoroughly with toluene. The filtrate was evaporated in vacuo to afford 2.4 g of crude. Compound 6A (2.40 g, crude) was obtained as a yellow oil. TLC (Petroleum ether:Ethyl acetate=1:1) R_{f}=0.48.

### 7. Preparation of Compound 5′-(S)—C-Me-S′—ODMTr-2′-OMe-U

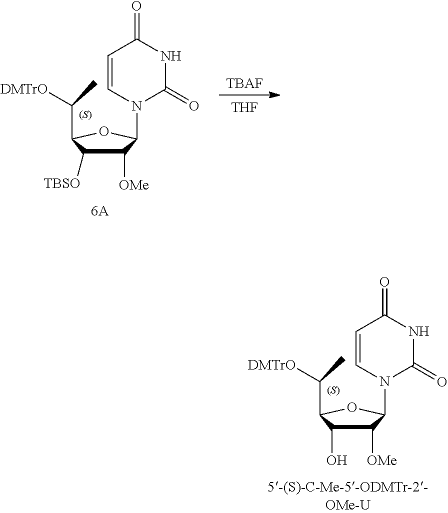

To a solution of compound 6A (1.32 g, 1.92 mmol) in THF (12.00 mL) was added TBAF (1 M, 3.64 mL). The mixture was stirred at 25° C. for 3 hours. TLC showed compound 6A was consumed and one new spot was detected. The mixture was concentrated under reduced pressure to give a residue. The residue was dissolved by EtOAc (50 mL) and washed by NaCl (5%, aq. 50 mL), extracted with EtOAc (50 mL*3), dried over Na₂SO₄, filtered and concentrated under reduced pressure to give a residue. The residue was purified by MPLC (SiO₂, silica gel was washed by Petroleum ether (5% TEA), Ethyl acetate/Petroleum ether=0%; 20%; 50%; 70%, 80% to 100%). Compound 5′-(S)—C-Me-5′-ODMTr-2′-OMe-U (800.00 mg, 72.51% yield) was obtained as a white solid.

¹H NMR (400 MHz, DMSO-d₆) δ=11.42 (s, 1H), 7.62 (d, J=8.2 Hz, 1H), 7.43 (br d, J=7.6 Hz, 2H), 7.34-7.19 (m, 7H), 6.88 (dd, J=5.3, 8.7 Hz, 4H), 5.81-5.73 (m, 2H), 5.58 (d, J=8.1 Hz, 1H), 5.11 (d, J=6.7 Hz, 1H), 4.22-4.11 (m, 1H), 3.83-3.72 (m, 8H), 3.55 (quin, J=5.7 Hz, 1H), 3.37-3.35 (m, 3H), 0.69 (d, J=6.2 Hz, 3H);

¹³C NMR (101 MHz, DMSO-d₆) δ=163.35, 158.58, 158.55, 150.93, 146.56, 136.81, 136.70, 130.57, 128.41, 128.08, 113.47, 102.49, 86.37, 85.94, 69.64, 68.18, 57.99, 55.44, 17.66;

LCMS (M+Na⁺): 597.2, 97.26% purity;

TLC (Petroleum ether:Ethyl acetate=1:1) Rf=0.10.

### 8. Preparation of Compound 5′-(S)—C-Me-5′-ODMTr-2′-OMe-U-CNE-phosphoramidite

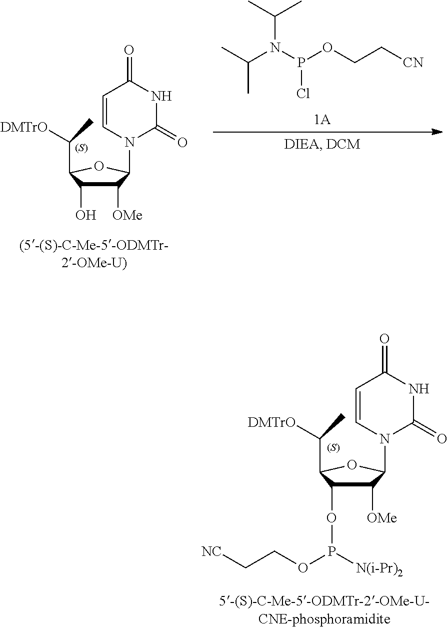

DIEA (1.32 g, 10.23 mmol, 1.79 mL) were added consecutively to a stirred solution of compound 1 (4.9 g, 8.53 mmol) in anhyd. DCM (50 mL) under Ar atm., and then added compound 1A (43.25 mg, 182.73 umol) at 0° C. After stirring at 0° C.-15° C. for 3 hr. LCMS showed compound 1 was partly remained and two major spots were detected. Then added compound 1A (201.82 mg, 852.74 umol), and after stirring at 0° C.-15° C. for 1 hr, TLC showed compound 1 was partly remained and two major spots were detected. The mixture was added sat. NaHCO₃ (aq., 20 mL) and extracted with DCM (50 mL*3). The combined organic layers were dried over Na₂SO₄, filtered and concentrated under reduced pressure to give a residue. The residue was purified by MPLC (SiO₂, Ethyl acetate/Petroleum ether=0%, 20%, 40%, 60%, 70%, 100%, 5% TEA) to give compound 5′-(S)—C-Me-5′-ODMTr-2′-OMe-U-CNE-phosphoramidite (4.5 g, 68.11% yield) was obtained as a white solid.

¹H NMR (400 MHz, CDCl₃) δ=8.56 (br s, 1H), 8.12-7.84 (m, 1H), 7.35-7.29 (m, 2H), 7.28-7.11 (m, 8H), 6.74 (ddd, J=3.0, 5.3, 8.6 Hz, 4H), 5.92 (t, J=4.0 Hz, 1H), 5.48 (t, J=8.1 Hz, 1H), 4.30-4.08 (m, 1H), 3.97-3.84 (m, 2H), 3.77-3.54 (m, 9H), 3.53-3.39 (m, 6H), 2.50 (t, J=6.2 Hz, 1H), 2.17 (t, J=6.3 Hz, 1H), 1.10-1.01 (m, 9H), 0.97-0.91 (m, 4H), 0.88 (br d, J=6.4 Hz, 2H)

³¹P NMR (162 MHz CDCl₃,) δ=150.40, 150.11, 14.16

LCMS: (M−H⁺): 773.3

HPLC purity: 40.4%+49.2%

TLC (Petroleum ether:Ethyl acetate=1:3, 5% TEA) R_{f1}=0.60, R_{f2}=0.55

### Example 34: Synthesis of 5′-(R)—C-Me-5′-ODMTr-dT

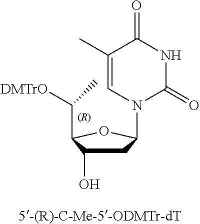

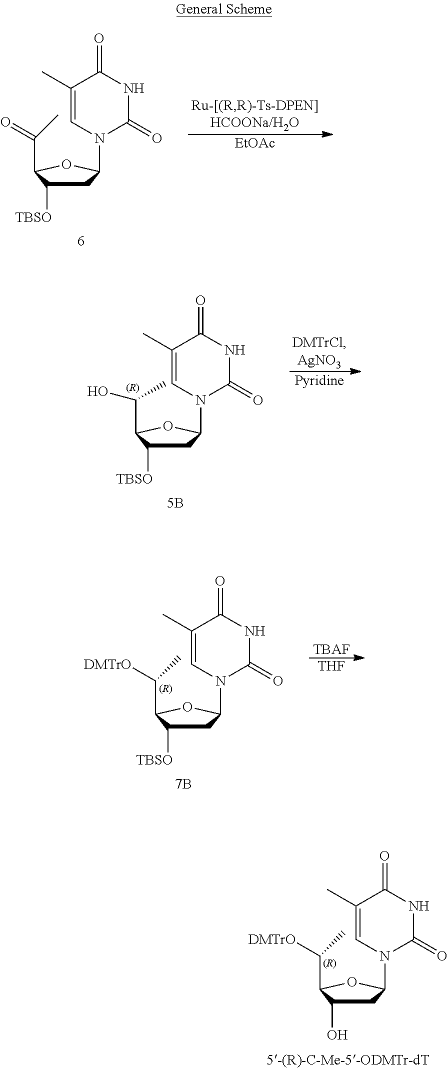

### 1. Preparation of Compound 5B

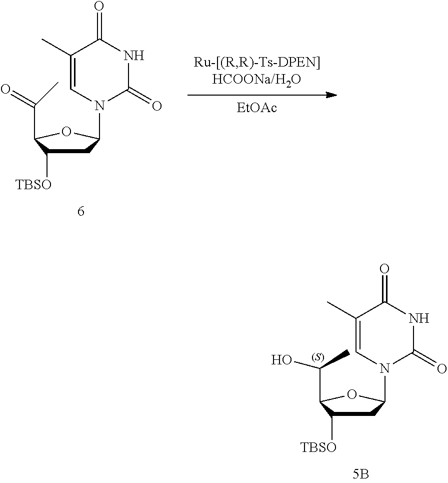

A 100 mL round-bottom flask equipped with a septum covered side arm was charged with [[(1R,2R)-2-amino-1,2-diphenyl-ethyl]-(p-tolylsulfonyl)amino]-chloro-ruthenium; 1-isopropyl-4-methyl-benzene (34.53 mg, 54.27 umol) and compound 6 (1.00 g, 2.71 mmol), and the system was flushed with nitrogen. A solution of sodium;formate;dihydrate (11.75 g, 112.89 mmol) in water (40.00 mL) was added, followed by EtOAc (10.00 mL). The resulting two-phase mixture was stirred for 12 h at 25° C. TLC showed the starting material was consumed. The mixture was extracted with EtOAc (50 mL*3). The combined organic was washed with brine (30 mL), dried over Na₂SO₄, filtered and concentrated to get the crude. The mixture was purified by MPLC (Petroleum ether/MTBE=10:1 to 1:1) to get compound 5B as a yellow oil (1.00 g, 99.50% yield).

¹H NMR (400 MHz, DMSO-d6): δ=11.30 (s, 1H), 7.67 (s, 1H), 6.16 (dd, J=5.6, 8.7 Hz, 1H), 5.04 (d, J=5.1 Hz, 1H), 4.49 (br d, J=5.1 Hz, 1H), 3.86-3.66 (m, 1H), 3.55 (d, J=4.2 Hz, 1H), 2.50 (br s, 12H), 2.22-2.05 (m, 1H), 1.96 (br dd, J=5.6, 12.9 Hz, 1H), 1.77 (s, 3H), 1.11 (d, J=6.2 Hz, 4H), 0.94-0.81 (m, 10H), 0.09 (s, 6H);

HPLC: HPLC purity: 84.4%;

TLC (Petroleum ether/Ethyl acetate=1:1) Rf=0.37.

### 2. Preparation of Compound 7B

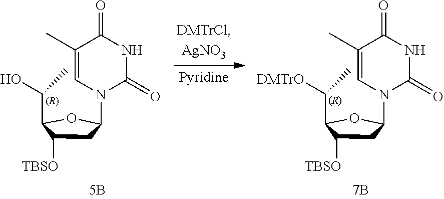

The compound 5B (1.00 g, 2.70 mmol) was dried by azeotropic distillation on a rotary evaporater with pyridine (20 mL) and toluene (20 mL*2).

A solution of compound 5B (1.00 g, 2.70 mmol) and DMTCl (1.89 g, 5.59 mmol) in the mixture of pyridine (10.00 mL) and THF (40.00 mL) was degassed and purged with N₂ for 3 times and then AgNO₃ (788.56 mg, 4.64 mmol) was added. The mixture was stirred at 25° C. for 15 hr. TLC showed the starting material was consumed. MeOH(1 mL) was added and stirred for 15 min and then the mixture was filtered and the cake was washed with toluene (20 mL*3), the filtrate was concentrated to get the compound 7B as a yellow oil (1.81 g, crude). The mixture was used directly to next step without any purification.

TLC (Petroleum ether/Ethyl acetate) Rf=0.63

### 3. Preparation of 5′-(R)—C-Me-5′-ODMTr-dT

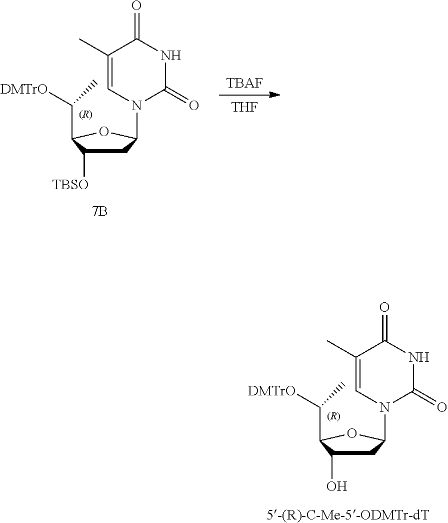

To a solution of compound 7B (1.81 g, 2.69 mmol.) in THF (20.00 mL) was added TBAF (1 M, 5.11 mL). The mixture was stirred at 25° C. for 3 hours. TLC showed the starting material was consumed. The mixture was concentrated to get the crude and then sat. NaCl (5% aq., 20 mL) was added and extracted with EtOAc (20 mL*3). The combined organic was dried over Na₂SO₄, filtered and concentrated to get the crude. The residue was purified by MPLC (Petroleum ether/Ethyl acetate 5:1, 1:1, 1:4, 5% TEA) to get 5′-(R)—C-Me-5′-ODMTr-dT as a white solid (1.00 g, 66.55% yield).

¹H NMR (400 MHz, DMSO-d6): δ=11.38 (s, 1H), 7.52 (d, J=7.5 Hz, 2H), 7.43-7.31 (m, 6H), 7.30-7.22 (m, 1H), 7.13 (d, J=1.0 Hz, 1H), 6.99-6.90 (m, 4H), 6.18 (t, J=7.2 Hz, 1H), 5.33 (d, J=4.8 Hz, 1H), 4.56 (quin, J=4.1 Hz, 1H), 3.79 (d, J=2.4 Hz, 6H), 3.68 (t, J=3.3 Hz, 1H), 3.47-3.39 (m, 1H), 2.11 (dd, J=4.8, 7.1 Hz, 2H), 1.46 (s, 3H), 0.83 (d, J=6.4 Hz, 3H)

HPLC: HPLC purity: 98.6%

LCMS: (M−H⁺)=557.2; LCMS purity: 100.0%

TLC (Petroleum ether/Ethyl acetate=1:1, 5% TEA) R_{f}=0.02.

### 4. Preparation of 5′-(R)—C-Me-5′-ODMTr-dT-CNE-phosphoramidite

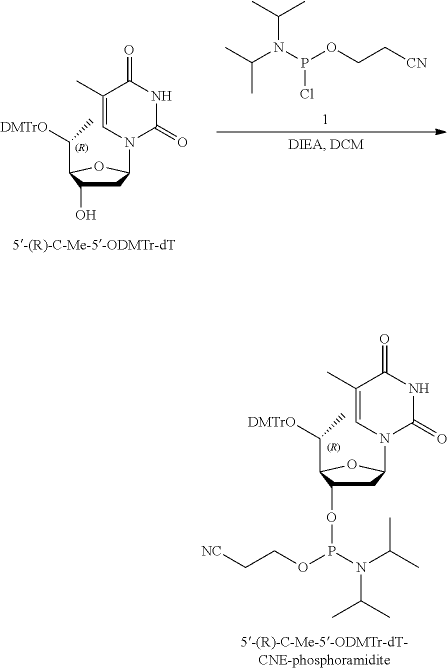

The 5′-(R)—C-Me-5′-ODMTr-dT (5 g, 8.95 mmol) was dried with toluene (50 mL). To a solution of DIEA (1.39 g, 10.74 mmol, 1.87 mL) and 5′-(R)—C-Me-5′-ODMTr-dT (5 g, 8.95 mmol) in anhyd. DCM (50 mL) was added compound 1 (2.76 g, 9.40 mmol) under N₂ at 0° C. The mixture was stirring at 15° C. for 2 h. TLC showed the starting material was consumed and two new spots were found. The mixture was quenched by addition of saturated aq. NaHCO₃ (20 mL) and extracted with DCM (30 mL*3). The combined organic was dried over Na₂SO₄, filtered and concentrated to get the crude. The above crude material was purified on a Combiflash instrument from Teledyne using either a pre-treated silica gel column. A 40 g silica gel cartridge column was first pre-treated by eluting with 10% EtOAc/Petroleum ether containing 5% Et₃N (300 mL) and the crude was dissolved in a 2:1 volume:volume mixture of methylene chloride: Petroleum ether containing 5% Et₃N then loaded onto a 40 g silica column which had been equilibrated with 10% Petroleum ether/EtOAc containing 5% Et₃N. After loading the sample on the column, the purification process was run using the following gradient: 10 to 50% EtOAc/Petroleum ether containing 5% Et₃N, then residual solvent was removed to get the 5′-(R)—C-Me-5′-ODMTr-dT-CNE-phosphoramidite as a white solid (3.6 g, 53.00% yield).

¹H NMR (400 MHz CDCl₃,) δ=8.11 (br s, 1H), 7.53 (br d, J=7.7 Hz, 3H), 7.42 (br t, J=8.2 Hz, 4H), 7.32-7.17 (m, 4H), 7.07-6.99 (m, 1H), 6.84 (br d, J=8.2 Hz, 4H), 6.31 (br dd, J=5.5, 8.7 Hz, 1H), 4.94 (br s, 1H), 3.96-3.73 (m, 10H), 3.72-3.41 (m, 4H), 2.65 (td, J=6.1, 18.0 Hz, 2H), 2.53-2.37 (m, 1H), 2.10 (br d, J=8.2 Hz, 1H), 1.47 (br s, 4H), 1.33-1.16 (m, 15H), 1.00-0.90 (m, 3H)

³¹P NMR (162 MHz, CDCl₃) δ=148.81 (s, 1P), 148.35 (s, 1P)

HPLC: HPLC purity: 59.15%+35.91%

LCMS: LCMS purity: 60.34%+37.17%

### Example 35: Synthesis of 5′-(S)—C-Me-5′-ODMTr-dT

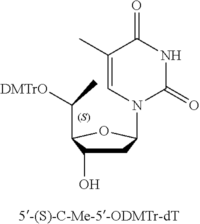

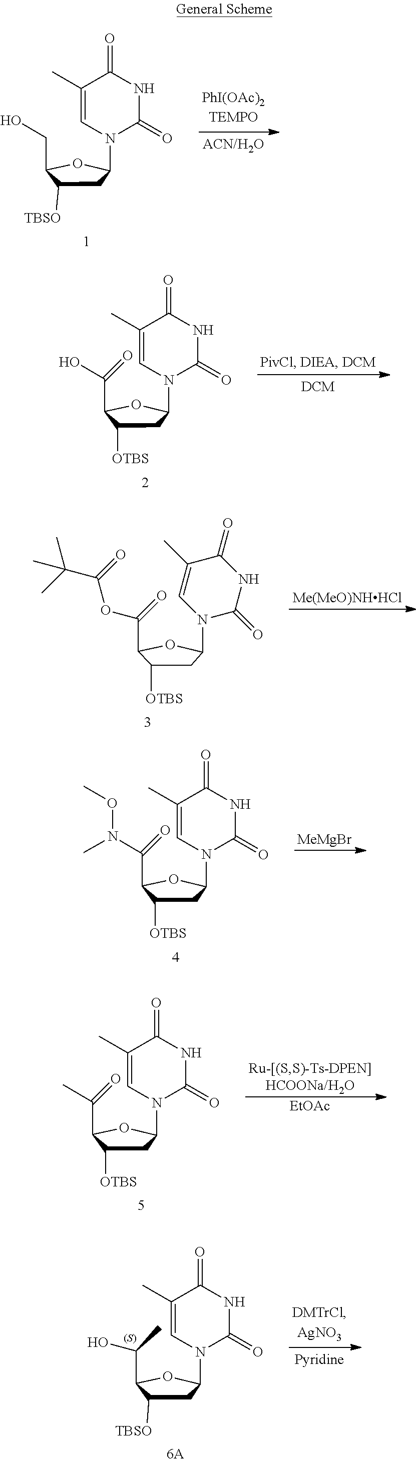

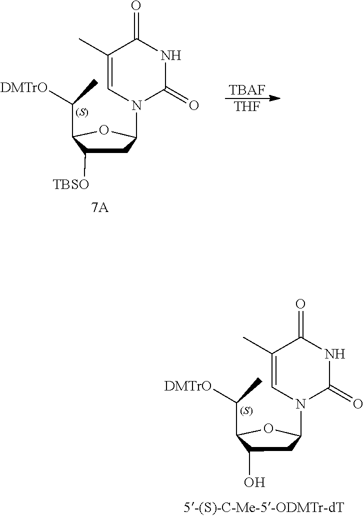

### 1. Preparation of Compound 2

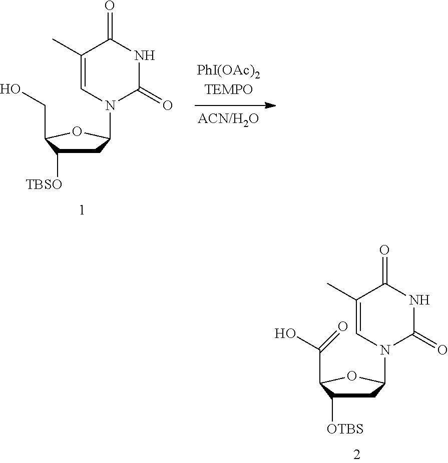

To a solution of compound 1 (63.00 g, 176.72 mmol) in the mixture of H₂O (250.00 mL) and MeCN (250.00 mL) was added PhI(OAc)₂ (125.23 g, 388.79 mmol) and TEMPO (5.56 g, 35.34 mmol) at 10° C. The mixture was stirred at 25° C. for 2 hour. TLC (Petroleum ether/Ethyl acetate=1:1, Rf=0) showed the starting material was consumed. The mixture was concentrated to get the crude and the mixture was added MTBE (1 L) stirred for 0.5 h and then filtered, the cake was washed with MTBE (1 L*2), the cake was dried to get the compound 2 as a white solid (126.00 g, 96.23% yield).

¹H NMR (400 MHz, DMSO): δ=11.21 (s, 1H), 7.89 (d, J=1.0 Hz, 1H), 6.18 (dd, J=5.9, 8.6 Hz, 1H), 4.61-4.41 (m, 1H), 4.17 (d, J=0.9 Hz, 1H), 2.51-2.26 (m, 3H), 2.09-1.85 (m, 2H), 1.74-1.58 (m, 3H), 0.90-0.58 (m, 10H), 0.00 (d, J=2.0 Hz, 6H)

LCMS: (M+H⁺): 371.1;

TLC (Petroleum ether/Ethyl acetate=1:1) Rf=0

### 2. Preparation of Compound 3

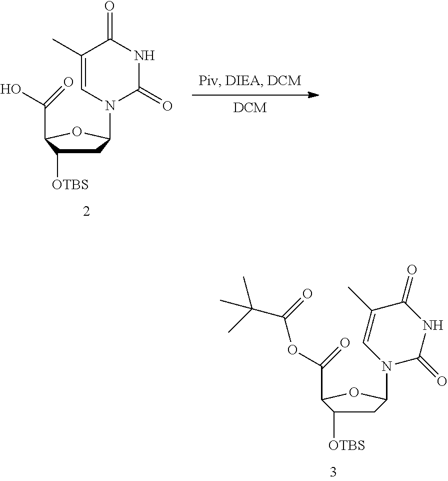

To a solution of compound 2 (50.00 g, 134.96 mmol) in DCM (500.00 mL) was added DIEA (34.89 g, 269.92 mmol, 47.15 mL) and 2,2-dimethylpropanoyl chloride (21.16 g, 175.45 mmol). The mixture was stirred at −10-0° C. for 1.5 hours. TLC showed the starting material was consumed. The mixture in DCM was used directly for next step. TLC (Petroleum ether/Ethyl acetate=1:1) Rf=0.15

### 3. Preparation of Compound-4

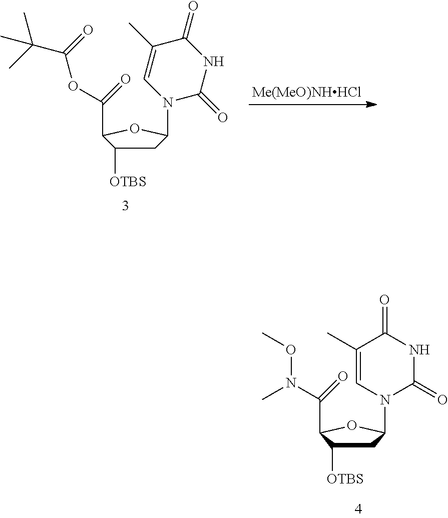

The mixture compound 3 in DCM was added TEA (40.94 g, 404.55 mmol, 56.08 mL) and N-methoxymethanamine hydrochloride (19.73 g, 202.27 mmol). The mixture was stirred at 0° C. for 1 h. TLC showed the starting material was consumed. The mixture was washed with HCl (1N, 100 mL) and then aqueous NaHCO₃ (100 mL). The organic was dried over Na₂SO₄ and filtered to get the crude. The mixture was purified by silica gel chromatography (Petroleum ether/Ethyl acetate=30/1, 0/1) to afford the compound 4 as a white solid (95.50 g, 85.63% yield).

¹H NMR (400 MHz, CDCl₃): δ=8.29 (s, 1H), 8.19 (br s, 1H), 6.46 (dd, J=5.1, 9.3 Hz, 1H), 4.71 (s, 1H), 4.38 (d, J=4.2 Hz, 1H), 3.65 (s, 3H), 3.15 (s, 3H), 2.18-2.08 (m, 1H), 2.00-1.90 (m, 1H), 1.87 (d, J=1.1 Hz, 3H), 0.88-0.74 (m, 10H), 0.00 (d, J=3.7 Hz, 6H) TLC (Petroleum ether/Ethyl acetate=1:1) R_{f}=0.43

### 4. Preparation of Compound 5

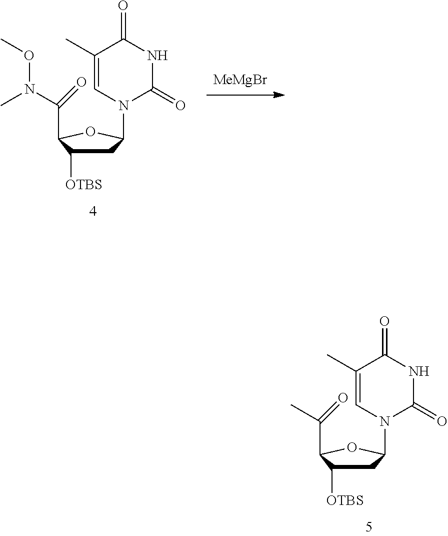

To a solution of compound 4 (115.00 g, 278.09 mmol) in THF (1.20 L) was added MeMgBr (3 M, 185.39 mL) at 0° C. The mixture was stirred at 0° C. for 2 h. TLC showed the starting material was consumed. The mixture was added water (1 L) at 0° C. and extracted with EtOAc (300 mL*2). The combined organic was dried over Na₂SO₄, filtered and concentrated to get the compound 5 as a white solid (100.00 g, 97.58% yield). The mixture was used directly without any purification.

¹H NMR (400 MHz, CDCl₃): δ=8.81 (br s, 1H), 7.95 (s, 1H), 6.41 (dd, J=5.6, 8.1 Hz, 1H), 4.60-4.40 (m, 2H), 2.40-2.16 (m, 4H), 1.98 (s, 3H), 1.02-0.83 (m, TOH), 0.14 (d, J=3.3 Hz, 6H), 0.20-0.00 (m, 1H) TLC (Petroleum ether/Ethyl acetate=1:1) R_{f}=0.68

### 5. Preparation of Compound 6A

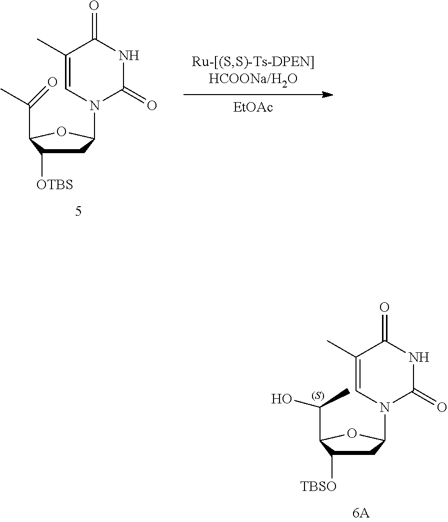

To a solution of compound 5 (46.00 g, 124.83 mmol) in the mixture of EtOAc (460.00 mL) and sodium formate (353.17 g, 5.19 mol) dissolved in Water (1.84 L), and then N— [(1S,2S)-2-amino-1,2-diphenyl-ethyl]-4-methyl-benzenesulfonamide;chlororuthenium;1-isopropyl-4-methyl-benzene (1.59 g, 2.50 mmol) was added. The resulting two-phase mixture was stirred for 12 h at 25° C. under N₂. TLC showed the starting material was consumed. The mixture was extracted with EtOAc (500 mL*3). The combined organic was washed with brine (300 mL), dried over Na₂SO₄, filtered and concentrated to get the crude. The mixture was purified by MPLC (Petroleum ether/MTBE=10:1 to 1:1) seven times to get compound 6A as a yellow oil (25.60 g, 57.53% yield).

¹H NMR (400 MHz, DMSO-d6): δ=11.28 (s, 1H), 7.85 (s, 1H), 6.16 (t, J=6.8 Hz, 1H), 5.04 (d, J=4.6 Hz, 1H), 4.46-4.29 (m, 1H), 3.79 (br t, J=6.8 Hz, 1H), 3.59 (br s, 1H), 3.32 (s, 1H), 2.21-2.09 (m, 1H), 2.06-1.97 (m, 1H), 1.76 (s, 3H), 1.17-1.08 (m, 4H), 0.91-0.81 (m, 10H), 0.08 (s, 6H)

SFC: SFC purity: 98.6%

TLC (Petroleum ether/Ethyl acetate=1:1) Rf=0.38

### 6. Preparation of Compound 7A

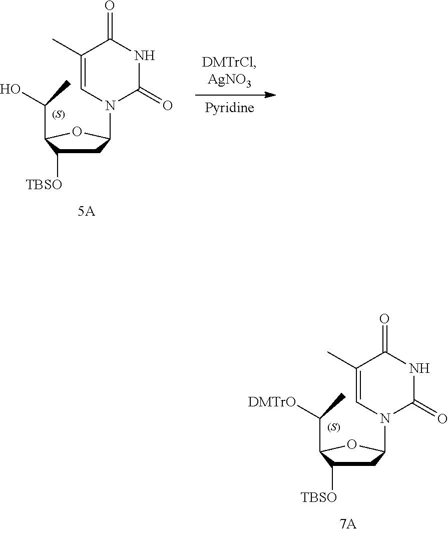

The compound 6A (12.80 g, 34.55 mmol) was dried by azeotropic distillation on a rotary evaporator with pyridine (100 mL) and toluene (100 mL*2).

To a solution of compound 6A (12.80 g, 34.55 mmol) and DMTCl (1.89 g, 5.59 mmol) in the mixture of pyridine (120.00 mL) and THF (400.00 mL) was degassed and purged with N₂ for 3 times and then AgNO₃ (10.09 g, 59.43 mmol) was added. The mixture was stirred at 25° C. for 15 hr. TLC showed the starting material was consumed. MeOH (5 mL) was added and stirred for 15 min and then the mixture was filtered and the cake was washed with toluene (300 mL*3). The filtrate was concentrated to get the compound 7A as a yellow oil (46.50 g, crude). The mixture was used directly to next step without any purification.

TLC (Petroleum ether/Ethyl acetate) Rf=0.63

### 7. Preparation of 5′-(S)—C-Me-5′-ODMTr-dT

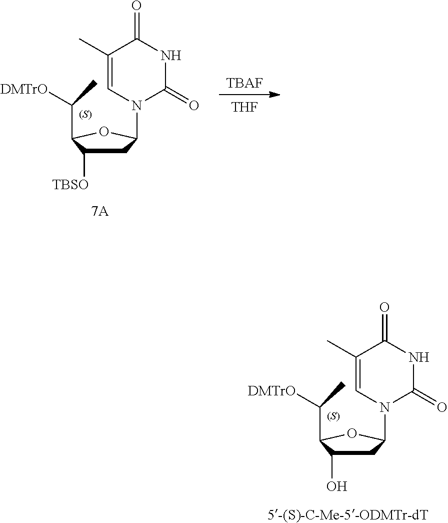

To a solution of compound 7A (46.50 g, 69.11 mmol) in THF (460.00 mL) was added TBAF (1 M, 131.31 mL). The mixture was stirred at 25° C. for 5 hrs. TLC showed the starting material was consumed. The mixture was concentrated to get the crude and then sat. NaCl (5% aq., 200 mL) was added and extracted with EtOAc (200 mL*3). The combined organic was dried over Na₂SO₄, filtered and concentrated to get the crude. The residue was purified by MPLC (Petroleum ether/Ethyl acetate 5:1, 1:1, 1:4, 5% TEA) to get 5′-(S)—C-Me-5′-ODMTr-dT as a white solid (29.00 g, 75.12% yield).

¹H NMR (400 MHz, DMSO-d6): δ=11.35 (s, 1H), 7.56 (s, 1H), 7.58-7.53 (m, 1H), 7.44 (d, J=7.8 Hz, 2H), 7.37-7.24 (m, 6H), 7.23-7.17 (m, 1H), 6.87 (t, J=8.3 Hz, 4H), 6.13 (t, J=6.9 Hz, 1H), 5.21 (d, J=4.9 Hz, 1H), 4.23 (br s, 1H), 3.73 (d, J=2.9 Hz, 6H), 3.67 (t, J=3.7 Hz, 1H), 3.57-3.46 (m, 1H), 2.23-2.04 (m, 2H), 1.67 (s, 3H), 1.70-1.65 (m, 1H), 0.71 (d, J=6.2 Hz, 3H)

¹³CNMR (101 MHz, DMSO-d6): δ=170.78, 164.16, 158.64, 158.59, 150.86, 146.71, 137.00, 136.75, 135.97, 130.65, 130.52, 128.38, 128.07, 127.11, 113.48, 110.11, 89.78, 86.41, 83.87, 70.58, 70.22, 60.21, 55.48, 21.20, 18.08, 14.53, 12.54

HPLC: HPLC purity: 98.4%

LCMS: (M−H⁺)=557.2; LCMS purity: 99.0%

SFC: SFC purity: 99.4%

TLC (Petroleum ether/Ethyl acetate=1:1, 5% TEA) R_{f}=0.01

### 8. Preparation of 5′-(S)—C-Me-5′-ODMTr-dT-CNE-phosphoramidite

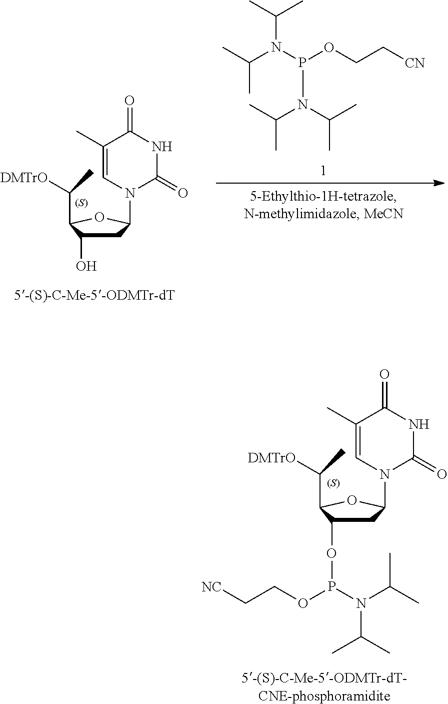

To a solution of 5′-(S)—C-Me-5′-ODMTr-dT (5.00 g, 8.95 mmol) in MeCN (50.00 mL) was added 5-ethylsulfanyl-2H-tetrazole (1.17 g, 8.95 mmol) 1-methylimidazole (1.47 g, 17.90 mmol, 1.43 mL) and compound 1 (4.05 g, 13.43 mmol, 4.26 mL). The reaction mixture was stirred at 20° C. under N₂ for 2 hrs. TLC and LCMS showed a little starting material was consumed and the desired substance was found. The reaction mixture was concentrated under reduced pressure to get the crude and the residue was diluted with EtOAc (20 mL). The reaction mixture was washed with aq. saturated. NaHCO₃ solution (20 mL), dried over Na₂SO₄, filtered and concentrated to get the crude. The mixture was purified by MPLC (Petroleum ether 5% TEA: Ethyl acetate from 10:1 to 1:1) we got two batches: 2.5 g (batch 1) and 1.8 g (batch 2). We got 5′-(S)—C-Me-5′-ODMTr-dT-CNE-phosphoramidite as a white solid (4.3 g, 5.67 mmol, 63.310% yield).

### Batch 1:

¹H NMR (400 MHz,) δ=8.19 (br s, 1H), 7.69-7.60 (m, 1H), 7.54 (s, 1H), 7.43-7.33 (m, 2H), 7.32-7.07 (m, 8H), 6.73 (ddd, J=3.7, 5.8, 9.0 Hz, 4H), 6.27-6.15 (m, 1H), 4.49-4.37 (m, 1H), 3.82-3.65 (m, 8H), 3.63-3.55 (m, 2H), 3.53-3.39 (m, 3H), 2.50 (t, J=6.3 Hz, 1H), 2.46-2.31 (m, 1H), 2.29-2.19 (m, 1H), 2.16-2.04 (m, 1H), 1.68 (s, 3H), 1.20-1.00 (m, 13H), 0.95 (d, J=6.8 Hz, 3H), 0.92-0.74 (m, 4H)

³¹P NMR (162 MHz, CDCl₃) δ=149.11 (s, 1P), 148.99 (s, 1P)

HPLC: HPLC purity: 62.68%+32.65%

LCMS: LCMS purity: 64.42%+32.87%

### Batch 2:

¹H NMR (400 MHz, CDCl₃) δ=8.19 (br s, 1H), 7.69-7.60 (m, 1H), 7.54 (s, 1H), 7.43-7.33 (m, 2H), 7.32-7.07 (m, 8H), 6.73 (ddd, J=3.7, 5.8, 9.0 Hz, 4H), 6.27-6.15 (m, 1H), 4.49-4.37 (m, 1H), 3.82-3.65 (m, 8H), 3.63-3.55 (m, 2H), 3.53-3.39 (m, 3H), 2.50 (t, J=6.3 Hz, 1H), 2.46-2.31 (m, 1H), 2.29-2.19 (m, 1H), 2.16-2.04 (m, 1H), 1.68 (s, 3H), 1.20-1.00 (m, 13H), 0.95 (d, J=6.8 Hz, 3H), 0.92-0.74 (m, 4H)

³¹P NMR (162 MHz, CDCl₃) δ=149.11 (s, 1P), 148.99 (s, 1P), 14.17 (s, 1P)

HPLC: HPLC purity: 53.0%+41.24%

LCMS: LCMS purity: 53.19%+42.83%

TLC (Petroleum ether/Ethyl acetate=1:3) R_{f}=0.86, 0.8

### Example 36: Synthesis of 3′-LPSE amidites

### General Procedure for the Preparation of 3′-LPSE Amidites

### Procedure for the Preparation of L-DPSE-Cl

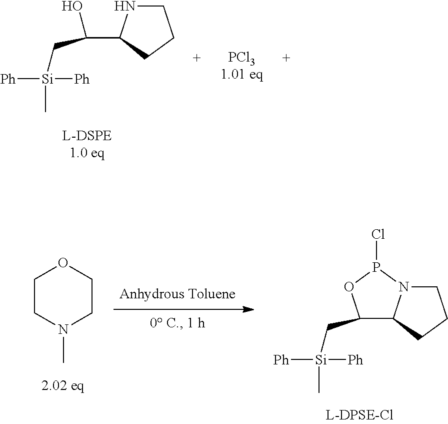

L-DPSE amino alcohol (S—2-(methyldiphenylsilyl)-1-((S)-pyrrolidin-2-yl)ethanol, 8.82 g, 28.5 mmol) was dried three times by azeotropic evaporation with anhydrous toluene (3×60 ml) at 35° C. and further dried in high vacuum for overnight. A solution of dried L-DPSE amino alcohol and 4-methylmorpholine (5.82 g, 6.33 mL, 57.5 mmole) which was dissolved in anhydrous toluene (50 ml) was added to a solution of PCl₃ (4.0 g, 2.5 mL, 29.0 mmole) in anhydrous toluene (25 ml) placed in 250 mL three neck round bottomed flask which was cooled at −5° C. under Argon. The reaction mixture was stirred at 0° C. for another 40 min. After that filtered the precipitated white solid by vacuum under argon using medium Frit, Airfree, Schlenk tube. The solvent was removed by under argon at low temperature (25° C.) and the semi solid mixture obtained was dried under vacuum overnight (˜15 h) and used for the next step directly.

³¹P NMR (162 MHz, CDCl₃) δ 178.84

### Procedure for Preparation of 3′-LPSE Amidites

Nucleosides (1.0 eq.) in an appropriate size three necked flask was azeotroped three times with anhydrous toluene (15 mL/g) and was dried for 24 h on high vacuum. To the flask was added anhydrous THF (0.3 M) under argon and solution was cooled to −10° C. To the reaction mixture was added triethylamine (5.0 eq.) followed by addition of L-DPSE-Cl (0.9 M solution in anhydrous THF, 1.7 eq.) over the period of 5-10 min. The reaction mixture was warmed to room temperature and reaction progress was monitored by LCMS. After disappearance of starting material, the reaction mixture was cooled in an ice bath and was quenched by addition of water (1.0 eq) stirred for 10 min followed by added anhydrous Mg₂SO₄ (1.0 eq) and stirred for 10 min. The reaction mixture was filtered through airfree fritted glass tube, washed with anhydrous THF (50 mL) and the solvent was removed under reduced pressure. The solid obtained was dried under high vacuum for overnight before purification. Then dried crude product was purified by silica column (which was pre-deactivated with 3 column volume of ethyl acetate with 5% TEA) using ethyl acetate/hexane mixture with 5% TEA as a solvent afforded 3′-L-DPSE amidites as a white solid.

### Preparation of 3′-L-DPSE-5′-PO(OMe)₂-Vinylphosphonate-dT amidite (3′-L-DPSE-WV—NU-010)

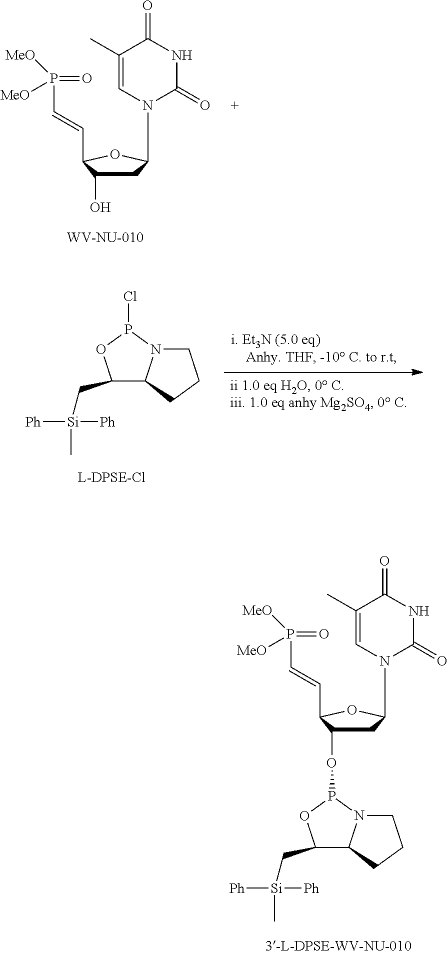

Nucleoside 5′-PO(OMe)₂-Vinylphosphonate-dT, WV-NU-010 (7.0 g) was converted to 3′-L-DPSE-5′-PO(OMe)₂-Vinylphosphonate-dT amidite (3′-L-DPSE-WV-NU-010) by general procedure and obtained 11.8 g (87%) as white solid.

³¹P NMR (162 MHz, CDCl₃) δ 152.41, 19.95.

¹H NMR (400 MHz, Chloroform-d) δ 7.46 (ddt, J=16.5, 7.6, 2.7 Hz, 4H), 7.33-7.17 (m, 6H), 6.93-6.88 (m, 1H), 6.75 (ddd, J=22.6, 17.2, 4.4 Hz, 1H), 6.16 (dd, J=7.5, 6.3 Hz, 1H), 5.85 (ddd, J=19.2, 17.1, 1.8 Hz, 1H), 4.71 (dt, J=8.7, 5.7 Hz, 1H), 4.38 (dp, J=10.7, 3.6 Hz, 1H), 4.15 (tt, J=5.6, 2.7 Hz, 1H), 3.68 (dd, J=11.1, 3.7 Hz, 6H), 3.55-3.29 (m, 2H), 3.09 (tdd, J=10.8, 8.8, 4.3 Hz, 1H), 2.11 (ddd, J=13.9, 6.3, 3.3 Hz, 1H), 1.96 (s, 1H), 1.87 (d, J=1.2 Hz, 3H), 1.85-1.73 (m, 2H), 1.70-1.49 (m, 2H), 1.38 (ddd, J=15.9, 10.4, 6.3 Hz, 2H), 1.26-1.11 (m, 2H), 0.60 (s, 3H).

¹³C NMR (101 MHz, CDCl₃) δ 171.07, 163.62, 163.59, 150.21, 150.19, 148.49, 148.43, 136.61, 135.84, 135.15, 134.57, 134.33, 129.48, 129.42, 127.97, 127.93, 127.81, 118.38, 116.50, 111.52, 85.02, 84.72, 84.70, 84.51, 84.48, 79.25, 79.16, 77.40, 77.28, 77.08, 76.76, 74.93, 74.91, 74.83, 74.81, 68.01, 67.98, 60.35, 52.60, 52.55, 52.47, 52.42, 47.03, 46.67, 38.12, 38.08, 27.18, 25.85, 25.82, 21.01, 17.58, 17.54, 14.19, 12.58, −3.00, −3.27.

LCMS: Chemical Formula: C₃₂H₄₁N₃O₈P₂Si; Calcd Molecular Weight: 685.72; Observed Molecular Weight: 684.68 [M−H]; 686.58 [M+H].

### Preparation of 3′-L-DPSE-5′-PO(OEt)₂-Vinylphosphonate-dT amidite (3′-L-DPSE-WV—NU-017)

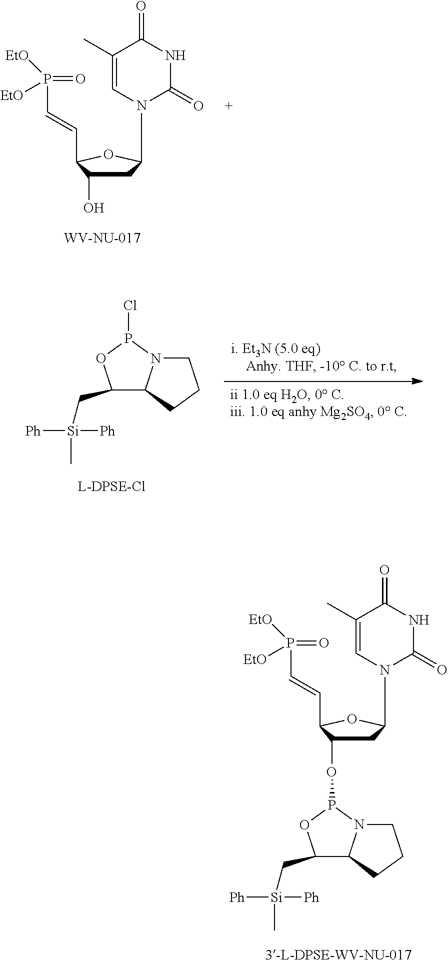

Nucleoside 5′-PO(OEt)₂-Vinylphosphonate-dT, WV-NU-017 (8.0 g) was converted to 3′-L-DPSE-5′-PO(OEt)₂-Vinylphosphonate-dT amidite (3′-L-DPSE-WV-NU-017) by general procedure and obtained 13.5 g (88%) as white crystalline solid.

³¹P NMR (162 MHz, CDCl₃) δ 152.44, 17.41.

¹H NMR (400 MHz, Chloroform-d) δ 9.56 (s, 1H), 7.61-7.46 (m, 5H), 7.40-7.26 (m, 7H), 7.00 (d, J=1.4 Hz, 1H), 6.81 (ddd, J=21.9, 17.1, 4.3 Hz, 1H), 6.27 (dd, J=7.6, 6.2 Hz, 1H), 5.96 (ddd, J=19.1, 17.1, 1.8 Hz, 1H), 4.79 (dt, J=8.8, 5.7 Hz, 1H), 4.46 (dp, J=10.3, 3.4 Hz, 1H), 4.24 (tt, J=5.6, 2.8 Hz, 1H), 4.20-4.02 (m, 5H), 3.63-3.37 (m, 2H), 3.18 (tdd, J=10.8, 8.8, 4.3 Hz, 1H), 2.18 (ddd, J=13.9, 6.2, 3.2 Hz, 1H), 1.95 (d, J=1.2 Hz, 3H), 1.93-1.54 (m, 5H), 1.47 (dd, J=14.8, 5.9 Hz, 2H), 1.39-1.16 (m, 8H), 0.69 (s, 3H).

¹³C NMR (101 MHz, CDCl₃) δ 171.09, 163.91, 163.77, 163.75, 150.32, 150.15, 147.57, 147.51, 136.66, 136.62, 136.09, 135.81, 135.08, 134.84, 134.59, 134.57, 134.49, 134.40, 134.32, 129.48, 129.42, 129.37, 127.98, 127.93, 127.90, 127.81, 119.77, 117.89, 111.89, 111.49, 85.86, 84.88, 84.80, 84.78, 84.59, 84.56, 79.24, 79.15, 78.91, 78.81, 77.45, 77.33, 77.13, 76.81, 74.94, 74.93, 74.85, 74.83, 68.02, 67.99, 62.08, 62.02, 61.96, 61.91, 61.87, 60.36, 47.15, 47.03, 46.80, 46.67, 45.92, 38.15, 38.11, 27.18, 27.14, 25.85, 25.81, 24.16, 21.03, 17.58, 17.54, 16.52, 16.46, 16.44, 16.40, 16.38, 14.20, 12.63, 12.42.

LCMS: Chemical Formula: C₃₄H₄₅N₃O₈P2Si; Calcd Molecular Weight: 713.78; Observed Molecular Weight: 712.27 [M−H); 714.26 [M+H].

### Preparation of 3′-L-DPSE-5′-PO(OEt)₂-Triazolylphosphonate-dT amidite (3′-L-DPSE-WV—NU-040)

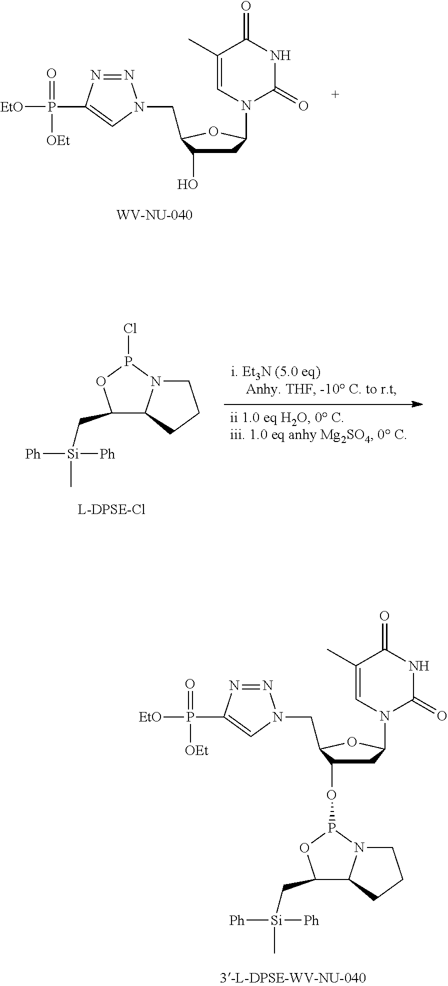

Nucleoside, 5′-PO(OEt)₂-Triazolylphosphonate-dT, WV-NU-040 (8.5 g) was converted to 3′-L-DPSE-5′-PO(OEt)₂-Triazolylphosphonate-dT amidite (3′-L-DPSE-WV-NU-040) by general procedure and obtained 10.5 g (69%) as a white solid.

³¹P NMR (162 MHz, Chloroform-d) δ 151.88, 6.69.

¹H NMR (400 MHz, Chloroform-d) δ 8.08 (d, J=1.8 Hz, 1H), 7.61-7.48 (m, 4H), 7.33 (dpt, J=6.5, 4.2, 2.1 Hz, 6H), 6.70 (d, J=1.5 Hz, 1H), 5.87 (dd, J=7.3, 6.2 Hz, 1H), 4.89-4.79 (m, 1H), 4.64 (ddd, J=14.7, 7.8, 4.3 Hz, 2H), 4.49 (dd, J=14.5, 6.4 Hz, 1H), 4.33-4.20 (m, 3H), 4.20-4.08 (m, 1H), 3.95 (td, J=5.9, 3.4 Hz, 1H), 3.66-3.42 (m, 2H), 3.20 (tddd, J=10.9, 8.9, 4.5, 2.1 Hz, 1H), 2.22-1.98 (m, 3H), 1.94 (q, J=1.2 Hz, 3H), 1.83-1.61 (m, 2H), 1.55-1.42 (m, 2H), 1.42-1.21 (m, 8H), 0.69 (d, J=1.6 Hz, 3H).

¹³C NMR (101 MHz, CDCl₃) δ 171.12, 163.64, 149.91, 138.68, 136.65, 136.58, 136.30, 135.88, 134.60, 134.48, 134.45, 134.36, 132.19, 131.86, 129.45, 129.40, 127.95, 127.93, 111.58, 86.98, 82.80, 79.41, 79.32, 77.39, 77.07, 76.75, 71.86, 71.77, 68.07, 68.04, 63.08, 63.05, 63.02, 62.99, 60.38, 50.51, 47.04, 46.68, 37.85, 37.81, 27.22, 25.85, 25.81, 21.04, 17.60, 17.56, 16.31, 16.25, 14.20, 12.43, −3.23, −3.81.

LCMS: Chemical Formula: C₃₅H₄₆N₆O₈P₂Si; Calcd Molecular Weight: 768.81; Observed Molecular Weight: 767.16 [M−H; 769.05 [M+H].

### Preparation of 3′-L-DPSE-5′-(R)-Me-PO(OEt)₂Phosphonate-dT amidite (3′-L-DPSE-WV—NU-037)

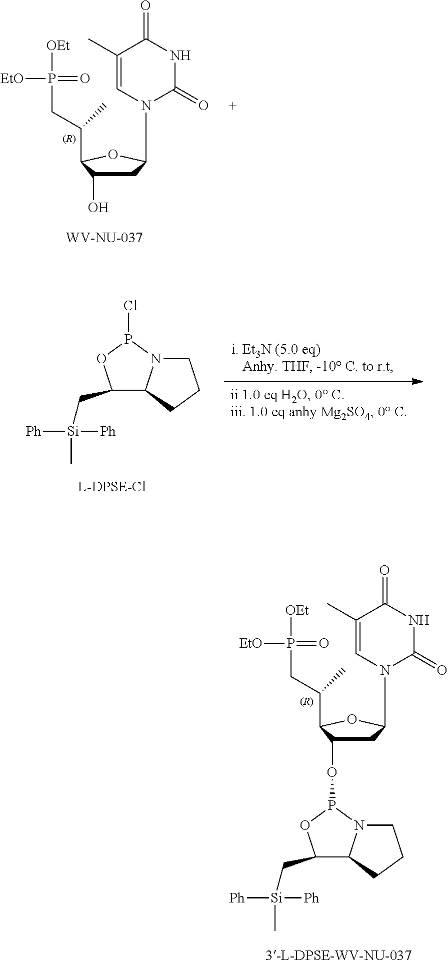

Nucleoside, 5′-(R)-Me-PO(OEt)₂ Phosphonate-dT, WV-NU-037 (8.0 g) was converted to 3′-L-DPSE-5′-(R)-Me-PO(OEt)₂ Phosphonate-dT amidite (3′-L-DPSE-WV-NU-037) by general procedure and obtained 12.5 g (86%) as a white solid.

³¹P NMR (162 MHz, Chloroform-d) δ 148.87, 30.96.

¹H NMR (400 MHz, Chloroform-d) δ 7.60-7.54 (m, 2H), 7.54-7.48 (m, 2H), 7.41-7.26 (m, 6H), 6.99 (t, J=1.3 Hz, 1H), 6.09 (dd, J=8.1, 5.9 Hz, 1H), 4.77 (dt, J=8.8, 5.7 Hz, 1H), 4.47 (tt, J=7.3, 3.0 Hz, 1H), 4.21-4.02 (m, 4H), 3.64-3.54 (m, 2H), 3.46 (ddd, J=12.7, 10.3, 5.9 Hz, 1H), 3.17 (qd, J=11.0, 4.2 Hz, 1H), 2.20-1.99 (m, 3H), 1.99-1.85 (m, 5H), 1.79-1.68 (m, 1H), 1.68-1.41 (m, 5H), 1.38-1.27 (m, 7H), 1.27-1.21 (m, 1H), 1.12 (d, J=6.6 Hz, 3H), 0.69 (d, J=1.0 Hz, 3H).

¹³C NMR (101 MHz, CDCl₃) δ 163.87, 150.22, 136.74, 135.88, 135.18, 134.63, 129.41, 129.38, 129.16, 128.18, 128.09, 127.94, 127.92, 111.19, 88.94, 88.91, 88.75, 88.72, 83.78, 79.60, 79.50, 77.45, 77.13, 76.81, 72.39, 72.35, 68.28, 68.25, 61.63, 61.59, 61.57, 61.52, 46.88, 46.52, 39.05, 31.35, 29.61, 28.20, 27.33, 25.84, 25.81, 17.79, 16.58, 16.53, 16.51, 16.47, 16.45, 12.67.

LCMS: Chemical Formula: C₃₅H₄₉N₃O₈P₂Si; Calcd Molecular Weight: 729.82; Observed Molecular Weight: 728.40 [M−H; 730.39 [M+H].

### Preparation of 3′-L-DPSE-5′-(S)-Me-PO(OEt)₂Phosphonate-dT amidite (3′-L-DPSE-WV—NU-037A)

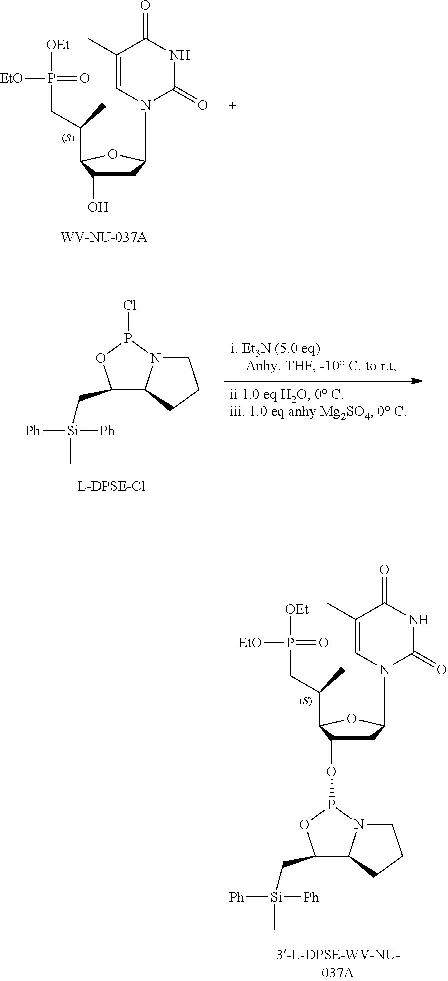

Nucleoside, 5′-(S)-Me-PO(OEt)₂ Phosphonate-dT, WV-NU-037A (10.0 g) was converted to 3′-L-DPSE-5′-(S)-Me-PO(OEt)₂ Phosphonate-dT amidite (3′-L-DPSE-WV-NU-037A) by general procedure and obtained 14.0 g (72%) as a white solid.

³¹P NMR (162 MHz, CDCl₃) δ 148.87, 30.96.

¹H NMR (400 MHz, Chloroform-d) δ 7.60-7.54 (m, 2H), 7.54-7.48 (m, 2H), 7.41-7.26 (m, 6H), 6.99 (t, J=1.3 Hz, 1H), 6.09 (dd, J=8.1, 5.9 Hz, 1H), 4.77 (dt, J=8.8, 5.7 Hz, 1H), 4.47 (tt, J=7.3, 3.0 Hz, 1H), 4.21-4.02 (m, 4H), 3.64-3.54 (m, 2H), 3.46 (ddd, J=12.7, 10.3, 5.9 Hz, 1H), 3.17 (qd, J=11.0, 4.2 Hz, 1H), 2.20-1.99 (m, 3H), 1.99-1.85 (m, 5H), 1.79-1.68 (m, 1H), 1.68-1.41 (m, 5H), 1.38-1.27 (m, 7H), 1.27-1.21 (m, 1H), 1.12 (d, J=6.6 Hz, 3H), 0.69 (d, J=1.0 Hz, 3H).

¹³C NMR (101 MHz, CDCl₃) δ 163.87, 150.28, 136.68, 135.93, 135.27, 135.23, 134.59, 134.44, 134.35, 129.43, 129.39, 127.95, 127.93, 111.45, 89.22, 89.19, 89.06, 89.03, 84.07, 79.21, 79.11, 77.42, 77.11, 76.79, 73.45, 73.37, 68.17, 68.14, 61.71, 61.65, 61.41, 61.34, 47.02, 46.66, 38.86, 38.83, 32.45, 32.41, 29.16, 27.76, 27.24, 25.83, 25.80, 17.73, 17.70, 17.12, 17.10, 16.51, 16.50, 16.45, 16.43, 16.42, 12.45.

LCMS: Chemical Formula: C₃₅H₄₉N₃O₈P₂Si; Calcd Molecular Weight: 729.82; Observed Molecular Weight: 728.40 [M−H; 730.39 [M+H].

### Preparation of L-DPSE-5′-ODMTr-5′-(R)-Me-2′F-dU amidite

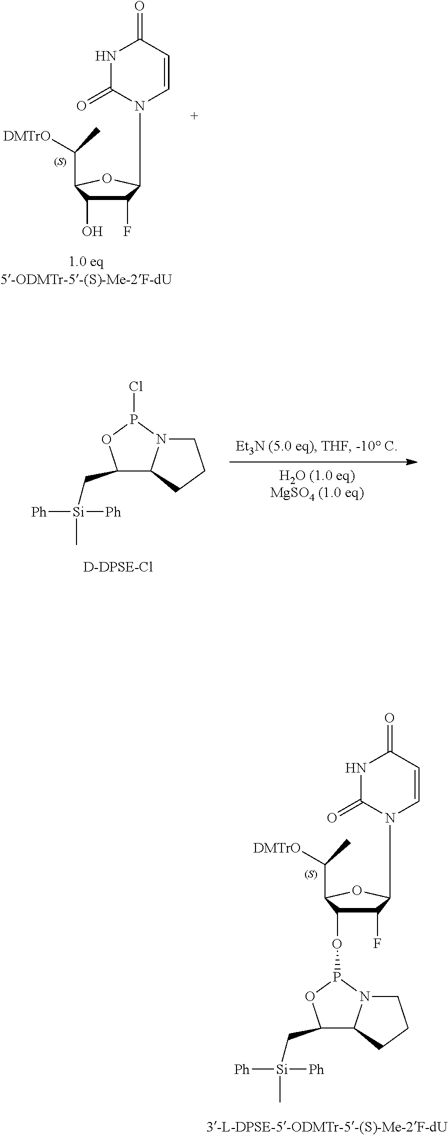

Nucleoside, 5′-ODMTr-5′-(R)-Me-2′F-dU (10 g) was converted to L-DPSE-5′-ODMTr-5′-(R)-Me-2′F-dU amidite by general procedure and obtained 14.0 g (87%) as a white crystalline solid.

³¹P NMR (243 MHz, CDCl₃) δ 151.48

¹H NMR (600 MHz, Chloroform-d) δ 7.57-7.45 (m, 6H), 7.41-7.24 (m, 12H), 7.23-7.18 (m, 1H), 7.16 (d, J=8.1 Hz, 1H), 6.86-6.80 (m, 4H), 5.79 (dd, J=17.4, 3.2 Hz, 1H), 5.19 (dd, J=8.0, 2.2 Hz, 1H), 4.97-4.86 (m, 2H), 4.13 (q, J=7.1 Hz, 1H), 3.78 (d, J=6.0 Hz, 6H), 3.74-3.70 (m, 1H), 3.61-3.53 (m, 2H), 3.49 (ddt, J=12.7, 10.4, 6.9 Hz, 1H), 3.10 (tdd, J=10.9, 8.9, 4.4 Hz, 1H), 2.56 (qd, J=7.2, 1.2 Hz, 1H), 2.05 (s, 1H), 1.93-1.84 (m, 1H), 1.76-1.69 (m, 1H), 1.66 (dd, J=14.6, 8.2 Hz, 1H), 1.51 (dd, J=14.6, 6.5 Hz, 1H), 1.43 (ddt, J=12.3, 7.6, 4.5 Hz, 1H), 1.34-1.24 (m, 2H), 1.04 (t, J=7.2 Hz, 2H), 0.88 (d, J=6.7 Hz, 3H), 0.66 (s, 3H.

¹³C NMR (151 MHz, CDCl₃) δ 171.20, 163.35, 163.32, 158.70, 158.60, 149.83, 149.82, 146.25, 141.18, 136.56, 136.31, 136.15, 135.99, 134.62, 134.40, 130.60, 130.40, 129.45, 129.43, 128.19, 127.95, 127.94, 127.86, 126.90, 113.21, 113.14, 102.48, 92.33, 91.05, 88.59, 88.37, 87.15, 85.36, 85.35, 79.63, 79.57, 77.34, 77.13, 76.91, 68.97, 68.61, 68.56, 68.51, 68.46, 68.01, 68.00, 60.44, 55.28, 55.25, 53.50, 46.74, 46.50, 45.96, 45.95, 27.27, 25.94, 25.92, 21.09, 17.97, 17.94, 17.14, 14.25, 11.33, 11.31, −3.34

¹⁹F NMR (565 MHz, CDCl₃) δ−199.82.

LCMS: Chemical Formula: C₅₀H₅₃FN₃O₈P₂Si; Calcd Molecular Weight: 902.04; Observed Molecular Weight: 901.03 [M−H]; 903.25 [M+H].

### Preparation of L-DPSE-5′-ODMTr-5′-(S)-Me-2′F-dU amidite

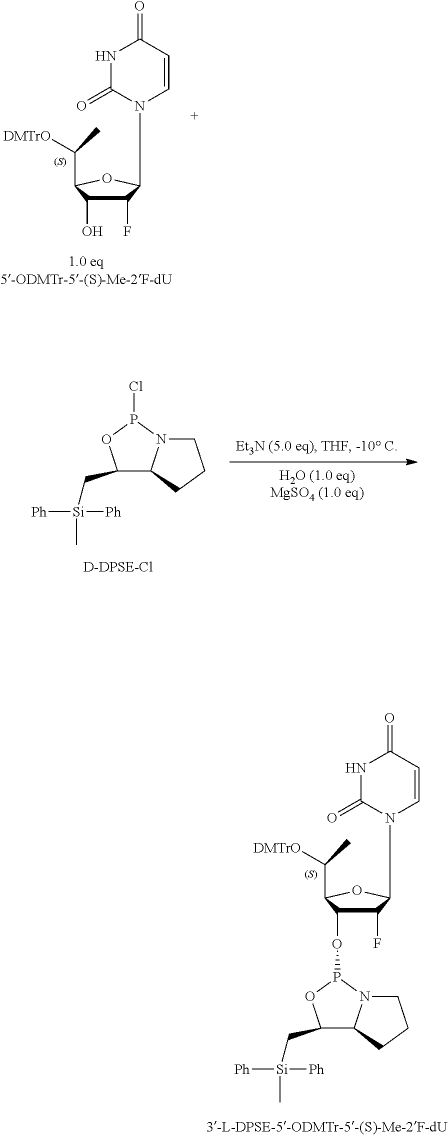

Nucleoside, 5′-ODMTr-5′-(S)-Me-2′F-dU (8 g) was converted to L-DPSE-5′-ODMTr-5′-(R)-Me-2′F-dU amidite by general procedure and obtained 10.0 g (78%) as a white crystalline solid.

³¹P NMR (243 MHz, CDCl₃) δ 150.98.

¹H NMR (600 MHz, Chloroform-d) δ 7.55 (d, J=8.1 Hz, 1H), 7.42 (ddd, J=13.2, 7.7, 1.7 Hz, 4H), 7.37-7.32 (m, 2H), 7.28-7.19 (m, 10H), 7.16 (t, J=7.5 Hz, 2H), 7.13-7.07 (m, 1H), 6.75-6.69 (m, 4H), 5.67 (dd, J=17.6, 2.1 Hz, 1H), 5.55 (d, J=8.1 Hz, 1H), 4.74-4.67 (m, 1H), 4.41 (dtd, J=16.2, 7.6, 4.9 Hz, 1H), 4.03 (q, J=7.1 Hz, 1H), 3.79 (dd, J=7.3, 3.7 Hz, 1H), 3.67 (d, J=5.1 Hz, 6H), 3.59 (qd, J=6.3, 3.6 Hz, 1H), 3.39 (ddt, J=14.5, 10.7, 7.5 Hz, 1H), 3.25 (ddd, J=12.3, 8.1, 4.9 Hz, 1H), 2.93 (tdd, J=10.8, 8.7, 4.5 Hz, 1H), 1.95 (s, 2H), 1.70 (dtt, J=12.3, 8.0, 3.7 Hz, 1H), 1.60-1.45 (m, 2H), 1.33 (dd, J=14.5, 6.5 Hz, 1H), 1.26 (dtd, J=12.5, 6.5, 3.2 Hz, 1H), 1.17 (t, J=7.1 Hz, 2H), 1.12 (dt, J=11.9, 8.0 Hz, 1H), 0.78 (d, J=6.3 Hz, 3H), 0.54 (s, 3H).

LCMS: Chemical Formula: C₅₀H₅₃FN₃O₈P₂Si; Calcd Molecular Weight: 902.04; Observed Molecular Weight: 901.05 [M−H]; 903.15 [M+H].

### Preparation of 3′-L-DPSE-5′-PO(OEt)₂-Abasic Vinylphosphonate (3′-L-DPSE-WV-RA-009)

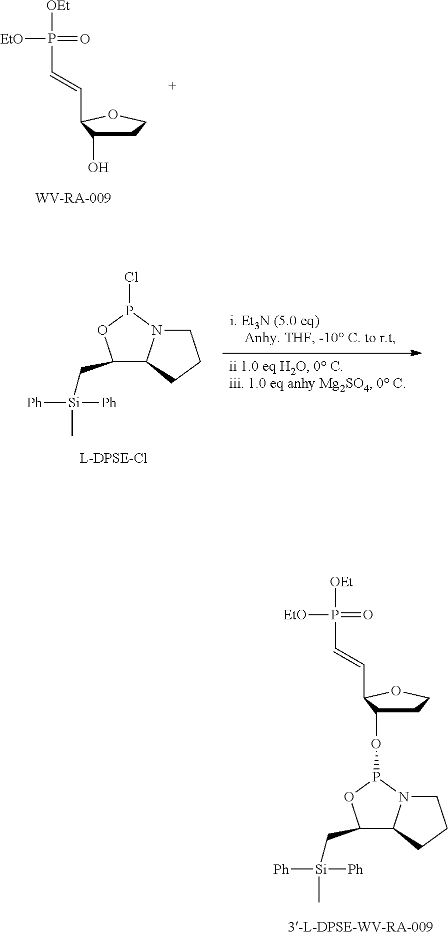

Diethyl((E)-2-((2R,3S)-3-hydroxytetrahydrofuran-2-yl)vinyl)phosphonate, (5′-PO(OEt)₂-Abasic Vinyl phosphonate, WV-RA-009 (5.0 g) was converted to 3′-L-DPSE-5′-PO(OEt)₂-Abasic Vinyl phosphonate (3′-L-DPSE-WV-RA-009) by general procedure and obtained 8.6 g (72.8%) as colorless semisolid.

³¹P NMR (243 MHz, CDCl₃) δ 152.94, 18.49.

¹H NMR (600 MHz, Chloroform-d) δ 7.47 (ddt, J=14.2, 6.6, 1.7 Hz, 8H), 7.33-7.24 (m, 11H), 6.65 (ddd, J=22.2, 17.0, 3.7 Hz, 2H), 5.85 (ddd, J=20.9, 17.0, 1.9 Hz, 2H), 4.73 (dt, J=8.5, 5.8 Hz, 2H), 4.26 (ddt, J=8.3, 5.4, 2.7 Hz, 2H), 4.16 (tt, J=3.6, 2.2 Hz, 2H), 4.08-3.94 (m, 8H), 3.91-3.81 (m, 4H), 3.47 (ddt, J=14.9, 10.6, 7.6 Hz, 2H), 3.32 (ddt, J=9.8, 7.6, 5.5 Hz, 2H), 3.14-3.05 (m, 2H), 1.82-1.78 (m, 1H), 1.75 (ddd, J=9.3, 7.4, 4.4 Hz, 5H), 1.67-1.58 (m, 2H), 1.55 (dd, J=14.7, 8.6 Hz, 2H), 1.41-1.34 (m, 3H), 1.34-1.30 (m, 1H), 1.28-1.19 (m, 11H), 1.19-1.12 (m, 2H), 0.60 (s, 5H).

LCMS: Chemical Formula: C₂₉H₄₁NO₆P₂Si; Calcd Molecular Weight: 589.68; Observed Molecular Weight: 588.63 [M−H]; 590.70 [M+H].

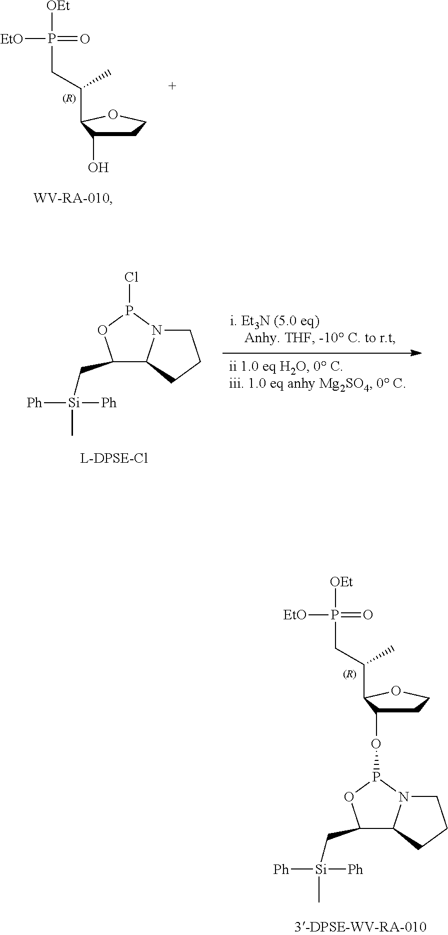

Diethyl((R)-2-((2R,3S)-3-hydroxytetrahydrofuran-2-yl)propyl)phosphonate, (5′-(R)-Me-PO(OEt)₂-Abasic phosphonate, WV-RA-010 (5.0 g) was converted to 3′-L-DPSE-5′-(R)-Me-PO(OEt)₂-Abasic phosphonate (3′-L-DPSE-WV-RA-010) by general procedure and obtained 7.0 g (62%) as colorless semisolid.

³¹P NMR (243 MHz, CDCl₃) δ 150.48, 31.86.

¹H NMR (600 MHz, Chloroform-d) δ 7.47 (ddt, J=14.6, 6.1, 1.7 Hz, 5H), 7.34-7.25 (m, 7H), 4.73 (ddd, J=8.1, 6.5, 5.3 Hz, 1H), 4.28-4.21 (m, 1H), 4.08-3.94 (m, 4H), 3.75 (td, J=8.1, 2.7 Hz, 1H), 3.71-3.62 (m, 1H), 3.52-3.42 (m, 1H), 3.41 (dd, J=5.9, 3.3 Hz, 1H), 3.35-3.26 (m, 1H), 3.08 (dddd, J=11.7, 10.6, 8.8, 4.3 Hz, 1H), 2.01-1.89 (m, 2H), 1.89-1.82 (m, 1H), 1.82-1.73 (m, 1H), 1.73-1.63 (m, 2H), 1.63-1.59 (m, 2H), 1.59-1.53 (m, 1H), 1.46-1.28 (m, 4H), 1.23 (td, J=7.1, 1.1 Hz, 6H), 1.22-1.11 (m, 2H), 0.97 (d, J=6.8 Hz, 3H), 0.60 (s, 3H).

LCMS: Chemical Formula: C₃₀H₄₅NO₆P₂Si; Calcd Molecular Weight: 605.72; Observed Molecular Weight:604.42 [M−H]; 606.53[M+H].

### Example 37: Synthesis of D-DPSE Amidite

### General Procedure for Synthesis of D-DPSE Amidite

### Procedure for the Preparation of D-DPSE-Cl

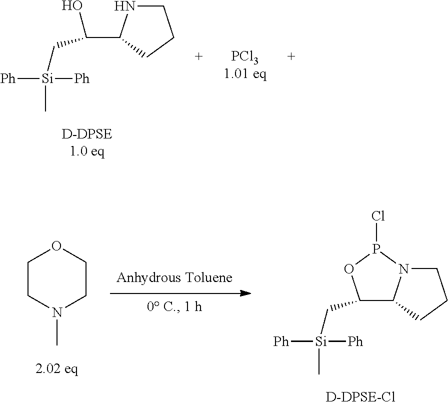

D-DPSE amino alcohol, ((R)-2-(methyldiphenylsilyl)-1-((R)-pyrrolidin-2-yl)ethanol (8.82 g, 28.5 mmol) was dried three times by azeotropic evaporation with anhydrous toluene (3×60 ml) at 35° C. and further dried in high vacuum for overnight. A solution of dried D-DPSE amino alcohol and 4-methylmorpholine (5.82 g, 6.33 mL, 57.5 mmole) which was dissolved in anhydrous toluene (50 ml) was added to a solution of PCl₃ (4.0 g, 2.5 mL, 29.0 mmole) in anhydrous toluene (25 ml) placed in 250 mL three neck round bottomed flask which was cooled at −5° C. under Argon. The reaction mixture was stirred at 0° C. for another 40 min. After that filtered the precipitated white solid by vacuum under argon using medium Frit, Airfree, Schlenk tube. The solvent was removed by rota-evaporator under argon at bath temperature (25° C.) and the crude oily mixture obtained was dried under vacuum overnight (˜15 h) and used for next step.

³¹P NMR (162 MHz, CDCl₃) δ 178.72,

### Procedure for Synthesis of D-DPSE Amidite.

Nucleosides (1.0 eq.) in an appropriate size three necked flask was azeotroped three times with anhydrous toluene (15 mL/g) and was dried for 24 h on high vacuum. To the flask was added anhydrous THF (0.3 M) under argon and solution was cooled to −10° C. To the reaction mixture was added triethylamine (5.0 eq.) followed by addition of D-DPSE-Cl (0.9 M solution in anhydrous THF, 1.7 eq.) over the period of 5-10 min. The reaction mixture was warmed to room temperature and reaction progress was monitored by LCMS. After disappearance of starting material, the reaction mixture was cooled in an ice bath and was quenched by addition of water (1.0 eq) stirred for 10 min followed by added anhydrous Mg₂SO₄ (1.0 eq) and stirred for 10 min. The reaction mixture was filtered through airfree fritted glass tube, washed with anhydrous THF (50 mL) and the solvent was removed under reduced pressure. The solid obtained was dried under high vacuum for overnight before purification. Then dried crude product was purified by silica column (which was pre-deactivated with 3 column volume of ethyl acetate with 5% TEA) using ethyl acetate/hexane mixture with 5% TEA as a solvent afforded 3′-D-DPSE amidites as a white solid.

### Preparation of 3′-D-DPSE-5′-ODMTr-5′-(R)-Me-dT amidite

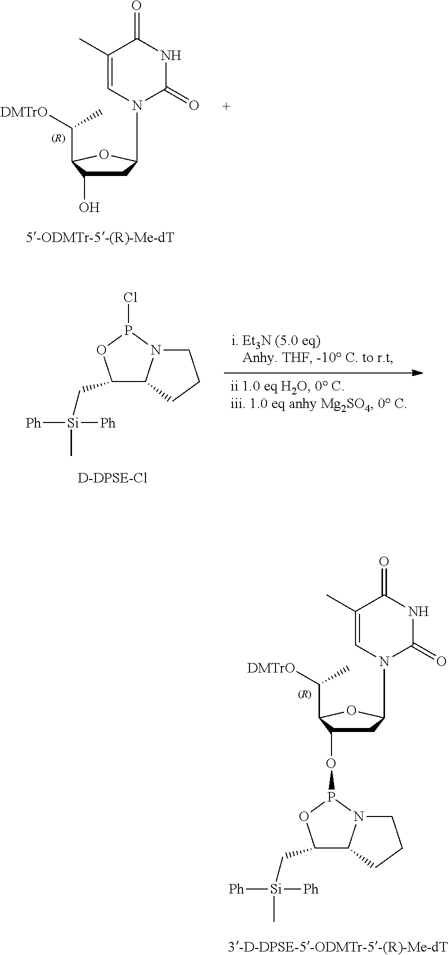

Nucleoside 5′-ODMTr-5′-(R)-Me-dT (10.0 g) was converted to 3′-D-DPSE-5′-ODMTr-5′-(R)-Me-dT amidite by general procedure (12.8 g, 90% yield) as an off white solid.

³¹P NMR (243 MHz, CDCl₃) δ=156.36

¹H NMR (600 MHz, CDCl₃) δ 8.94-8.75 (m, 1H), 7.52-7.38 (m, 4H), 7.31 (dd, J=13.6, 8.6 Hz, 4H), 7.27-7.21 (m, 4H), 7.21-7.15 (m, 2H), 7.14-7.07 (m, 1H), 6.86 (d, J=1.8 Hz, 1H), 6.74 (dd, J=8.9, 3.8 Hz, 4H), 6.07 (t, J=7.2 Hz, 1H), 4.81 (ddt, J=11.8, 9.0, 4.5 Hz, 2H), 3.69 (d, J=3.0 Hz, 7H), 3.48 (ddd, J=15.1, 7.5, 2.7 Hz, 1H), 3.36 (dq, J=10.7, 3.8 Hz, 2H), 3.14 (dd, J=9.6, 4.0 Hz, 1H), 1.96 (d, J=1.2 Hz, 2H), 1.83-1.68 (m, 3H), 1.68-1.51 (m, 2H), 1.44 (dd, J=14.7, 6.0 Hz, 1H), 1.36 (s, 4H), 1.27-1.09 (m, 3H), 0.83 (d, J=6.5 Hz, 3H), 0.63 (s, 3H).

LCMS: C₅₁H₅₆N₃O₈PSi (M−H): 897.16

### Preparation of 3′-D-DPSE-5′-ODMTr-5′-(S)-Me-dT amidite

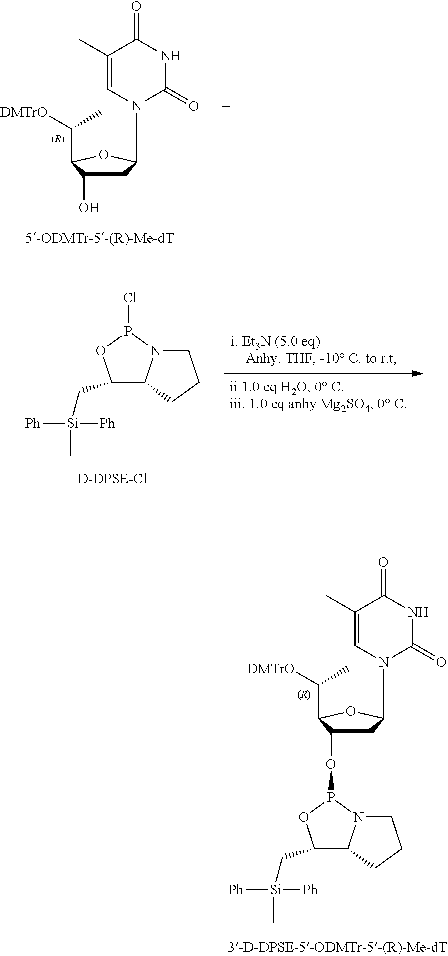

Nucleoside 5′-ODMTr-5′-(S)-Me-dT (8.0 g) was converted to 3′-D-DPSE-5′-ODMTr-5′-(S)-Me-dT amidite by general procedure (10 g, 89% yield) as an off white solid.

³¹P NMR (243 MHz, CDCl₃) δ=156.36

¹H NMR (600 MHz, CDCl₃) δ 8.81 (s, 1H), 7.60 (d, J=2.4 Hz, 1H), 7.49-7.41 (m, 4H), 7.41-7.36 (m, 2H), 7.33-7.28 (m, 2H), 7.29-7.21 (m, 7H), 7.21-7.15 (m, 2H), 7.12 (t, J=7.3 Hz, 1H), 6.73 (dd, J=8.9, 6.5 Hz, 4H), 6.11-6.03 (m, 1H), 4.68 (dt, J=8.7, 5.8 Hz, 1H), 4.52-4.44 (m, 1H), 3.70 (d, J=3.8 Hz, 6H), 3.65 (t, J=3.4 Hz, 1H), 3.49 (qd, J=6.5, 3.0 Hz, 1H), 3.34 (ddt, J=15.1, 10.1, 7.7 Hz, 1H), 3.30-3.22 (m, 1H), 3.08-2.98 (m, 1H), 1.89 (dt, J=14.1, 7.2 Hz, 1H), 1.81 (ddd, J=13.8, 6.2, 3.7 Hz, 1H), 1.76-1.68 (m, 4H), 1.63-1.48 (m, 2H), 1.38 (dd, J=14.7, 6.0 Hz, 1H), 1.31 (dtd, J=12.1, 6.4, 2.6 Hz, 1H), 1.21-1.10 (m, 3H), 0.83 (d, J=6.3 Hz, 3H), 0.58 (d, J=1.5 Hz, 3H).

LCMS: C₅₁H₅₆N₃O₈PSi (M−H): 897.16

### Preparation of 3′-D-DPSE-5′-ODMTr-5′-(R)-Me-2′F-dU amidite

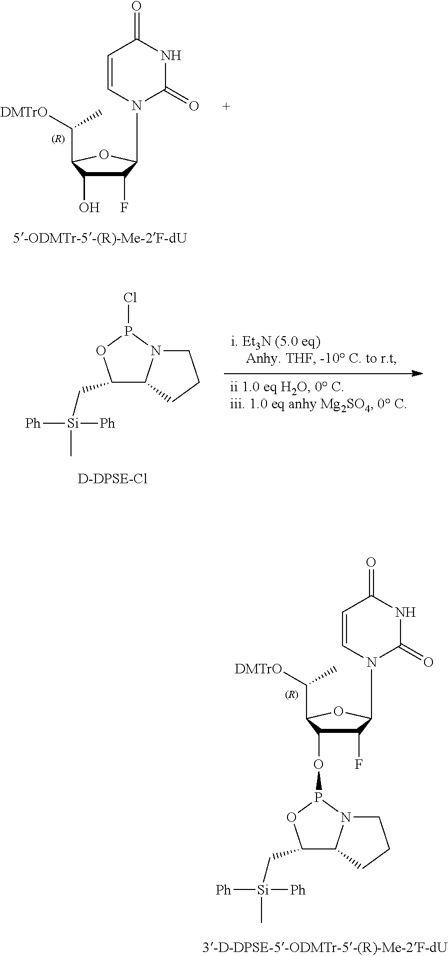

Nucleoside 5′-ODMTr-5′-(R)-Me-2′F-dU (5.0 g) was converted to 3′-D-DPSE-5′-ODMTr-5′-(R)-Me-2′F-dU amidite by general procedure (6.0 g, 75% yield) as an off white solid.

³¹P NMR (243 MHz, CDCl₃) δ=156.86

¹⁹F NMR (565 MHz, CDCl₃) δ+−198.88-−199.16 (m).

¹H NMR (600 MHz, CDCl₃) δ 9.23 (d, J=8.6 Hz, 1H), 7.51-7.43 (m, 4H), 7.43-7.36 (m, 2H), 7.35-7.29 (m, 2H), 7.30-7.20 (m, 7H), 7.17 (t, J=7.6 Hz, 2H), 7.11 (t, J=7.4 Hz, 1H), 5.81 (dd, J=17.6, 2.2 Hz, 1H), 5.04-4.88 (m, 2H), 4.82-4.70 (m, 1H), 3.80 (d, J=7.6 Hz, 1H), 3.69 (d, J=2.8 Hz, 6H), 3.54 (ddd, J=13.7, 9.3, 6.9 Hz, 2H), 3.36-3.27 (m, 1H), 3.21-3.11 (m, 1H), 1.80 (dp, J=12.5, 4.4 Hz, 1H), 1.62 (dd, J=14.7, 7.8 Hz, 2H), 1.41 (dd, J=14.7, 6.7 Hz, 1H), 1.30 (qd, J=7.5, 2.6 Hz, 1H), 1.25-1.14 (m, 3H), 0.87 (d, J=6.7 Hz, 3H), 0.59 (s, 3H).

LCMS: C₅₀H₅₃FN₃O₈PSi (M−H): 901.14

### Preparation of 3′-D-DPSE-5′-ODMTr-5′-(S)-Me-2′F-dU amidite

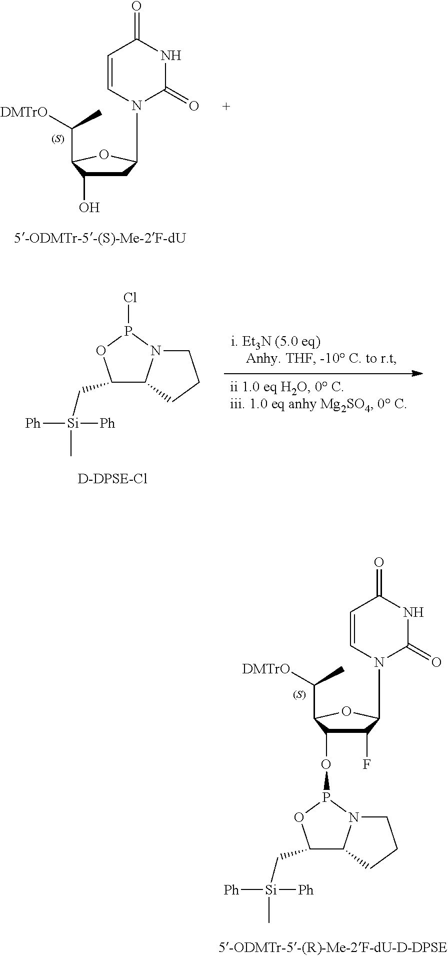

Nucleoside 5′-ODMTr-5′-(S)-Me-2′F-dU (4.95 g) was converted to 3′-D-DPSE-5′-ODMTr-5′-(S)-Me-2′F-dU amidite by general procedure (6.95 g, 87% yield) as an off white solid.

³¹P NMR (243 MHz, CDCl₃) δ=156.92

¹⁹F NMR (565 MHz, CDCl₃) δ=−198.87-−199.13 (m).

¹H NMR (600 MHz, CDCl₃) δ 9.65-9.28 (m, 1H), 7.90 (d, J=8.2 Hz, 1H), 7.44 (ddd, J=12.3, 7.7, 1.9 Hz, 4H), 7.36-7.30 (m, 2H), 7.30-7.19 (m, 7H), 7.17 (t, J=7.7 Hz, 2H), 7.12 (t, J=7.3 Hz, 1H), 6.72 (t, J=8.4 Hz, 4H), 5.87 (d, J=17.1 Hz, 1H), 5.53 (d, J=8.2 Hz, 1H), 4.87 (q, J=6.8 Hz, 1H), 4.69-4.53 (m, 1H), 4.51-4.40 (m, 1H), 3.86 (dd, J=8.6, 2.6 Hz, 1H), 3.69 (d, J=4.4 Hz, 6H), 3.52 (qd, J=6.4, 2.7 Hz, 1H), 3.36 (ddt, J=15.2, 10.2, 7.7 Hz, 1H), 3.23-3.14 (m, 1H), 3.05 (td, J=10.0, 3.8 Hz, 1H), 1.71 (dh, J=12.5, 3.9 Hz, 1H), 1.65-1.57 (m, 1H), 1.52 (dq, J=12.6, 8.2 Hz, 1H), 1.35 (dd, J=14.6, 7.5 Hz, 1H), 1.24-1.14 (m, 3H), 1.08 (q, J=10.2 Hz, 1H), 0.88 (d, J=6.5 Hz, 3H), 0.56 (s, 3H).

LCMS: C₅₀H₅₃FN₃O₈PSi (M−H): 901.14

### Preparation of 3′-D-DPSE-5′-PO(OEt)₂ Vinylphosphonate-dT amidite

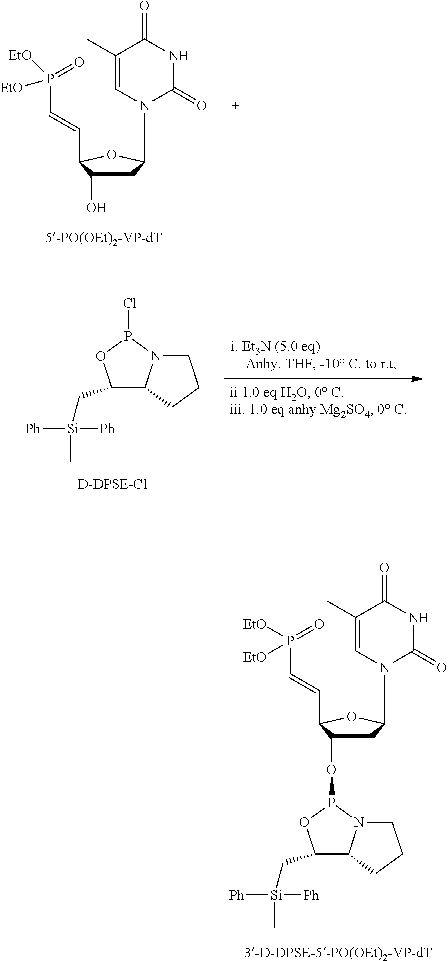

Nucleoside 5′-PO(OEt)₂ VP-dT (10 g) was converted to 3′-D-DPSE-5′-PO(OEt)₂ Vinyl phosphonate-dT amidite by general procedure (14.1 g, 73% yield) as an off white solid.

LCMS: C₃₄H₄₅N₃O₈P₂Si (M−H⁻). 712.45

¹H NMR (600 MHz, CDCl₃) δ 9.03 (s, 1H), 7.55-7.35 (m, 4H), 7.32-7.21 (m, 6H), 6.91 (s, 1H), 6.82-6.70 (m, 1H), 6.11 (t, J=6.7 Hz, 1H), 5.96-5.83 (m, 1H), 4.80-4.69 (m, 1H), 4.35-4.20 (m, 2H), 4.09-3.95 (m, 4H), 3.51-3.41 (m, 1H), 3.41-3.31 (m, 1H), 3.22-3.06 (m, 1H), 1.96 (d, J=6.7 Hz, 1H), 1.92-1.83 (m, 3H), 1.83-1.71 (m, 3H), 1.70-1.56 (m, 1H), 1.53 (dd, J=14.3, 8.7 Hz, 1H), 1.46-1.31 (m, 2H), 1.31-1.11 (m, 8H), 0.59 (d, J=6.9 Hz, 3H).

³¹P NMR (243 MHz, CDCl₃) δ=156.66, 17.09

### Preparation of 3′-D-DPSE-5′-(R)-Me-PO(OEt)₂-dT amidite

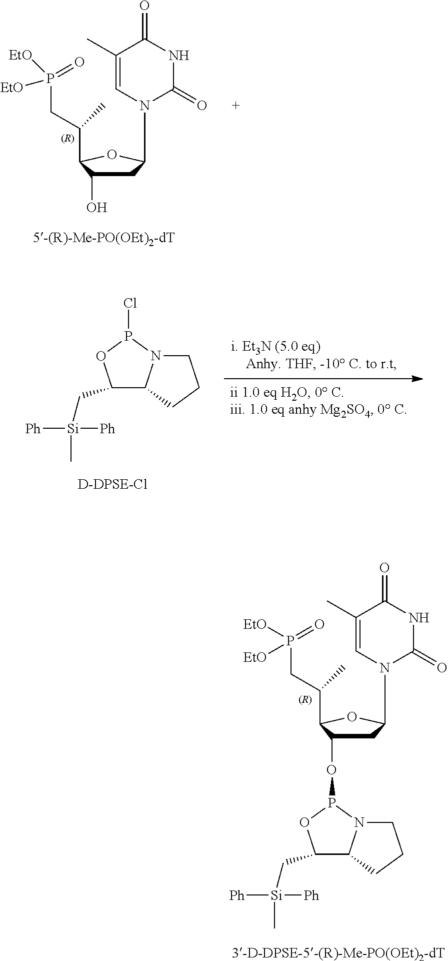

Nucleoside 5′-(R)-Me-PO(OEt)₂-dT (4.0 g) was converted to 3′-D-DPSE-5′-(R)-Me-PO(OEt)₂-dT amidite by general procedure (5.0 g, 69% yield) as an off white solid.

³¹P NMR (162 MHz, CDCl₃) δ 156.32, 30.68.

¹H NMR (400 MHz, Chloroform-d) δ 8.87 (d, J=56.9 Hz, 1H), 7.54 (ddt, J=16.6, 5.9, 2.4 Hz, 5H), 7.35 (t, J=3.4 Hz, 7H), 7.02 (d, J=1.4 Hz, 1H), 6.05 (t, J=6.8 Hz, 1H), 4.83 (dt, J=9.0, 5.7 Hz, 1H), 4.31 (tt, J=8.9, 4.6 Hz, 1H), 4.11 (tdt, J=10.2, 7.1, 5.1 Hz, 5H), 3.66 (t, J=5.2 Hz, 1H), 3.55 (ddd, J=15.2, 10.2, 7.5 Hz, 1H), 3.45 (ddt, J=13.4, 10.5, 5.6 Hz, 1H), 3.22 (tdd, J=11.1, 8.8, 4.2 Hz, 1H), 2.24 (dddt, J=12.8, 9.7, 6.2, 3.6 Hz, 1H), 2.06 (d, J=1.7 Hz, 1H), 2.03-1.57 (m, 12H), 1.55-1.40 (m, 2H), 1.38-1.20 (m, 9H), 1.15 (d, J=6.6 Hz, 3H), 0.68 (d, J=1.1 Hz, 3H).

¹³C NMR (101 MHz, CDCl₃) δ 163.50, 150.01, 136.71, 135.96, 135.17, 134.56, 134.37, 129.49, 129.38, 127.98, 127.91, 111.31, 88.34, 88.28, 88.16, 88.09, 83.29, 78.20, 78.12, 77.38, 77.06, 76.74, 72.22, 72.06, 67.71, 67.69, 61.64, 61.58, 61.56, 61.49, 60.38, 47.24, 46.90, 38.95, 30.84, 30.80, 30.16, 28.75, 27.10, 25.92, 25.89, 21.04, 17.27, 17.24, 16.51, 16.50, 16.46, 16.44, 15.99, 15.96, 14.20, 12.69,

LCMS: C₃₅H₄₉N₃O₈P₂Si (M−H): 728.21

### Preparation of 3′-D-DPSE-5′-(S)-Me-PO(OEt)₂-dT amidite

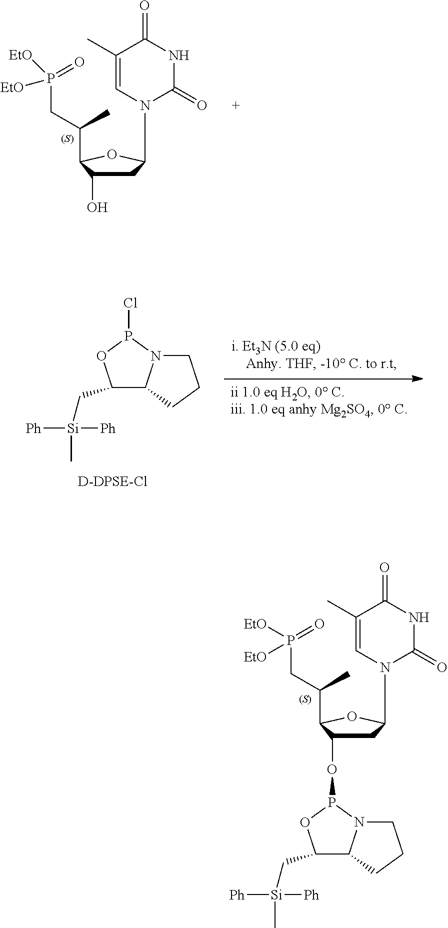

Nucleoside 5′-(S)-Me-PO(OEt)₂-dT (3.9 g) was converted to 3′-D-DPSE-5′-(S)-Me-PO(OEt)₂-dT amidite by general procedure (4.1 g, 56% yield) as an off white solid.

³¹P NMR (243 MHz, CDCl₃) δ=155.76, 31.56

¹H NMR (600 MHz, CDCl₃) δ 9.24 (s, 1H), 7.52-7.37 (m, 4H), 7.32-7.21 (m, 6H), 7.02 (s, 1H), 6.05 (t, J=7.1 Hz, 1H), 4.74 (dt, J=10.1, 5.7 Hz, 1H), 4.28-4.20 (m, 1H), 4.10-3.95 (m, 4H), 3.52-3.40 (m, 2H), 3.40-3.31 (m, 1H), 3.19-3.07 (m, 1H), 2.14-2.04 (m, 1H), 2.03-1.95 (m, 1H), 1.91 (s, 3H), 1.83-1.67 (m, 3H), 1.68-1.59 (m, 1H), 1.53 (dd, J=14.7, 9.0 Hz, 1H), 1.47-1.32 (m, 3H), 1.30-1.14 (m, 8H), 1.07 (d, J=6.7 Hz, 3H), 0.60 (s, 3H).

LCMS: C₃₅H₄₉N₃O₈P₂Si (M−H): 728.82

### Example 38: Synthesis of WV-NU-231

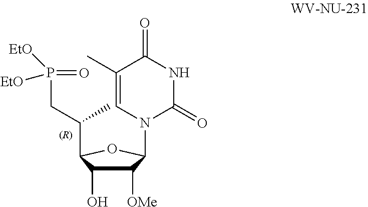

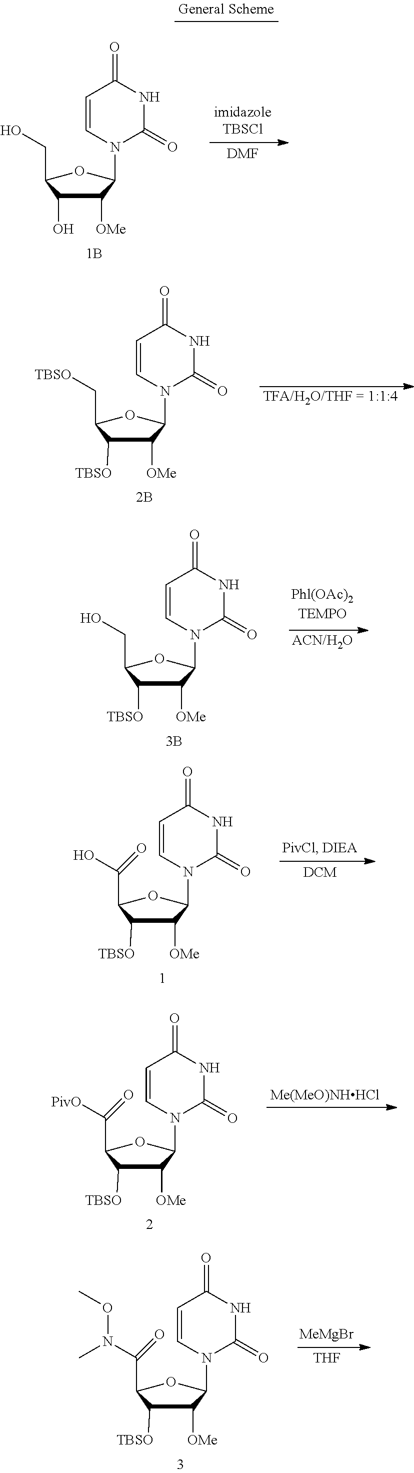

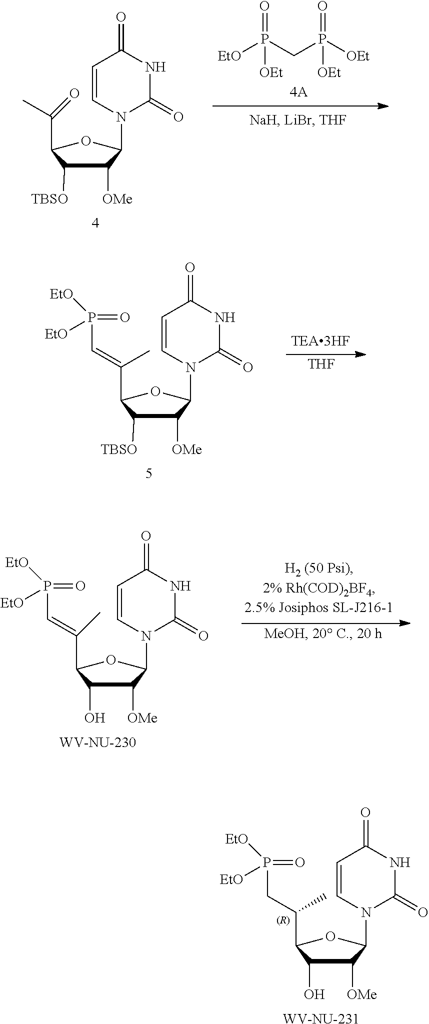

### 1. Preparation of Compound 2B

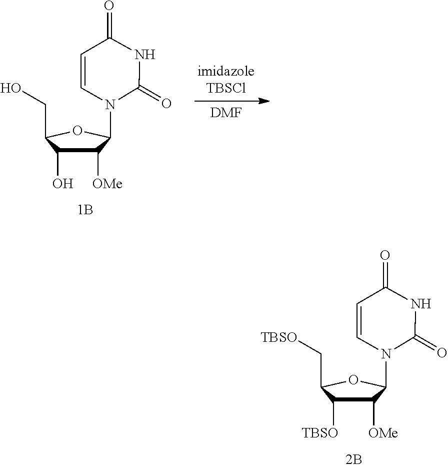

For two batches. To a solution of compound 1B (125 g, 484.07 mmol) in DMF (1000 mL) was added TBSCl (291.84 g, 1.94 mol.) and imidazole (164.78 g, 2.42 mol). The mixture was stirred at 20° C. for 12 hr. LCMS showed the desired mass was detected. The reaction mixture was diluted with H₂O 2000 mL and extracted with ethyl acetate 3000 mL (1000 mL*3). The combined organic layers were washed with brine 1000 mL, dried over Na₂SO₄, filtered and concentrated under reduced pressure to give a residue. The residue was purified by column chromatography (SiO₂, Petroleum ether/Ethyl acetate=1/0 to 0/1). Compound 2B (470 g, 99.74% yield) was obtained as a colorless oil.

1H NMR (400 MHz, DMSO-d₆) δ=11.38 (s, 1H), 7.79 (d, J=8.1 Hz, 1H), 5.80 (d, J=3.6 Hz, 1H), 5.55 (d, J=8.1 Hz, 1H), 4.22 (t, J=5.2 Hz, 1H), 3.92-3.81 (m, 3H), 3.73-3.63 (m, 1H), 3.37 (s, 3H), 0.91-0.85 (m, 18H), 0.08 (s, 12H)

LCMS (M−H+): 485.4

TLC (Ethyl acetate:Methanol=3:1), R_{f}=0.55

### 2. Preparation of Compound 3B

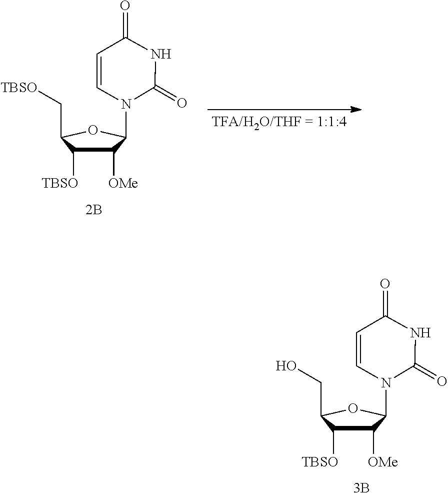

For three batches. To a stirred solution of compound 2B (166 g, 341.04 mmol) in THF (1412 mL) was added the mixture of TFA (353 mL) and H₂O (353 mL). The mixture was stirred at 0° C. for 3 hr. LCMS showed the desired mass was detected. The reaction mixtures of two batches were combined and neutralized with saturated aqueous NaHCO₃ and extracted with ethyl acetate 5L*3. The combined organic layers were washed with brine 2L*2, dried over anhydrous Na₂SO₄ and evaporated at reduced pressure. The residue was purified by column chromatography (SiO₂, Petroleum ether/Ethyl acetate1=1/0 to 0/1). Compound 3B (340 g, 89.22% yield) was obtained as a white solid.

1H NMR (400 MHz, DMSO-d6) δ=11.36 (s, 1H), 7.96 (d, J=8.1 Hz, 1H), 5.84 (d, J=5.0 Hz, 1H), 5.66 (dd, J=1.8, 8.1 Hz, 1H), 5.39 (br s, 1H), 4.32 (t, J=4.5 Hz, 1H), 3.90-3.80 (m, 2H), 3.71-3.62 (m, 1H), 3.60-3.52 (m, 1H), 3.33 (s, 3H), 0.95-0.81 (m, 9H), 0.08 (s, 6H)

LCMS (M−H+): 371.2

TLC (Petroleum ether:Ethyl acetate=1:1), Rf=0.32

### 3. Preparation of Compound 1

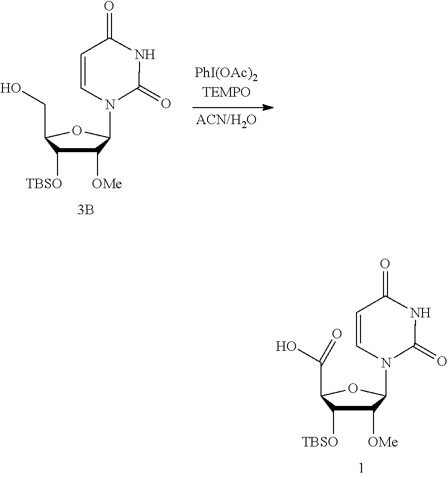

For three batches. To a solution of Compound 3B (70 g, 187.93 mmol, 1 eq.) in the mixture of ACN (500 mL) and H₂O (500 mL) was added PhI(OAc)₂ (133.17 g, 413.44 mmol, 2.2 eq.) and TEMPO (5.91 g, 37.59 mmol, 0.2 eq.). The mixture was stirred at 20° C. for 2 hr. LCMS showed the desired mass was detected. The resulting mixture was concentrated then filtrated, and the solid was desired product. Compound 1 (150 g, crude) was obtained as a white solid.

LCMS (M−H+): 385.3

### 4. Preparation of Compound 2

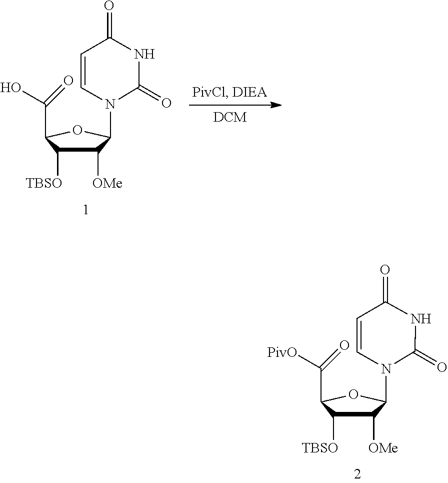

For three batches. To a solution of compound 1 (40 g, 103.50 mmol) in DCM (400 mL) was added DIEA (26.75 g, 207.00 mmol) and 2,2-dimethylpropanoyl chloride (16.22 g, 134.55 mmol). The mixture was stirred at −10˜0° C. for 2 hr. TLC indicated compound 1 was consumed completely and one new spot formed. The crude product compound 2 (146 g, crude) in 400 mL DCM was used into the next step without further purification.

TLC (Petroleum ether:Ethyl acetate=1:1), Rf=0.69

### 5. Preparation of Compound 3

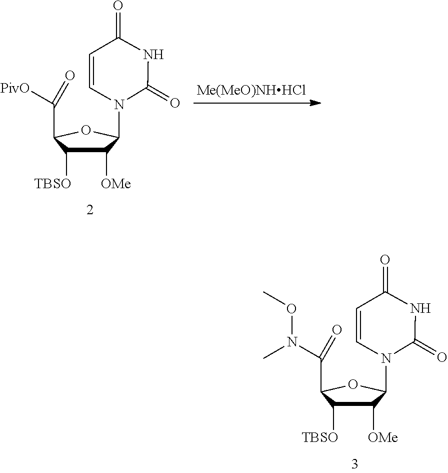

To a solution of Compound 2 (146 g, 310.25 mmol) in DCM 400 mL was added TEA (94.18 g, 930.75 mmol, 129.55 mL) then added N-methoxymethanamine;hydrochloride (90.79 g, 930.75 mmol). The mixture was stirred at 0° C. for 2 hr. TLC showed the desired mass was detected. The resulting mixture was washed with HCl (1M, 800 mL*2) and then aqueous NaHCO₃ (600 mL*2). The combined organic layers were dried over anhydrous Na₂SO₄, filtered and concentrated to get the product as a crude white solid. The residue was purified by column chromatography (SiO₂, Petroleum ether/Ethyl acetate=I/O to 0:1). Compound 3 (45 g, 33.77% yield) was obtained as a white solid.

TLC (Petroleum ether:Ethyl acetate=0:1), Rf=0.47

### 6. Preparation of Compound 4

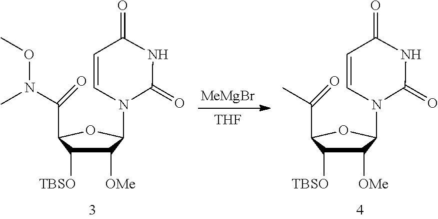

For three batches. To a solution of compound 3 (24 g, 55.87 mmol) in THF (300 mL) was added MeMgBr (3 M, 37.25 mL). The mixture was stirred at 0° C. for 1.5 hr. indicated compound 3 was consumed completely and new spot formed. The resulting mixture was poured into sat. NH₄Cl aq. (500 mL) under stirring, extracted with EtOAc (800 mL*3). The combined organic layers were dried over anhydrous Na₂SO₄, filtered and concentrated to give a crude. The residue was purified by column chromatography (SiO₂, Petroleum ether/Ethyl acetate=1/0 to 0:1). Compound 4 (53 g, 82.23% yield) was obtained as a white solid.

1H NMR (400 MHz, DMSO-d₆) δ=11.43 (s, 1H), 8.03 (d, J=8.1 Hz, 1H), 5.92 (d, J=5.9 Hz, 1H), 5.75 (d, J=8.1 Hz, 1H), 4.64-4.53 (m, 1H), 4.50 (d, J=3.5 Hz, 1H), 3.88-3.79 (m, 1H), 3.31 (s, 3H), 2.20 (s, 3H), 0.91 (s, 9H), 0.13 (d, J=3.1 Hz, 6H) TLC (Petroleum ether:Ethyl acetate=1:1), R_{f}=0.6

### 7. Preparation of Compound 5

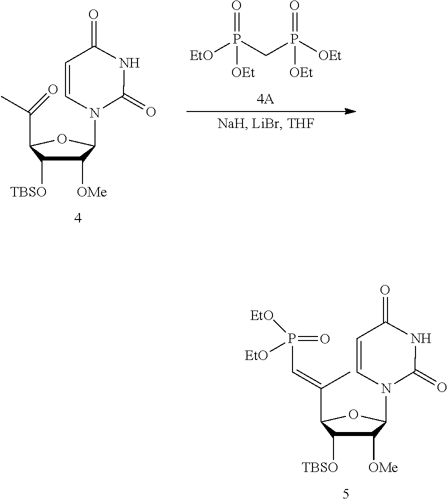

For five batches. To a solution of NaH (4.85 g, 121.30 mmol, 60% purity) in THF (50 mL) was added 1-[diethoxyphosphorylmethyl(ethoxy)phosphoryl]oxyethane (34.96 g, 121.30 mmol) in THF (400 mL) at 0° C. The reaction mixture was warmed up to 20° C., and stirred for 1 hr. A solution of LiBr (10.53 g, 121.30 mmol, 3.04 mL) in THF (100 mL) was added and the resultant slurry was stirred, and then cooled to 0° C. To the above mixture was added a solution of compound 4 (10.6 g, 27.57 mmol) in THF (100 mL) at 0° C. The mixture was stirred at 0-20° C. for 12 hr. LCMS indicated compound 4 was consumed completely and one new spot formed. The resulting mixture was diluted with water (1000 mL), extracted with EtOAc (1000 mL*3). The combined organic layers were washed with sat.brine (500 mL*2), dried over anhydrous Na₂SO₄, filtered and concentrated to afford the crude. Compound 5 (71 g, crude) was obtained as a colorless gum.

LCMS (M−H⁺): 517.4

### 8. Preparation of Compound WV-NU-230

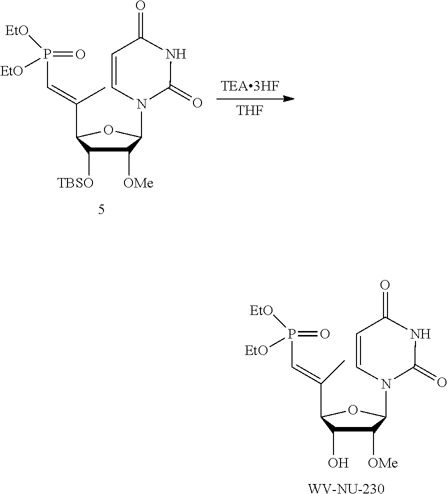

To a solution of compound 5 (71 g, 136.90 mmol) in THF (700 mL) was added N,N-diethylethanamine;trihydrofluoride (176.56 g, 1.10 mol, 178.53 mL). The mixture was stirred at 40° C. for 6 hr. LCMS showed compound 5 was consumed completely and one main peak with desired mass was detected. The reaction mixture was quenched by addition sat. NaHCO₃ aq. (500 mL) and NaHCO₃ solid to pH=7˜8 and stirred 20 min. The mixture was dried over Na₂SO₄, and concentrated under reduced pressure to give a residue. The residue was purified by column chromatography (SiO₂, Petroleum ether:Ethyl acetate=1/1 to 0/1 then Ethyl acetate: Methanol=I/O to 3/1). TLC (Ethyl acetate:Methanol=10:1, Rf=0.3). Compound WV-NU-230 (42.5 g, 76.77% yield) was obtained as a colorless gum.

¹H NMR (400 MHz, DMSO-d₆) δ=11.44 (s, 1H), 7.65 (d, J=8.0 Hz, 1H), 5.77 (d, J=4.4 Hz, 1H), 5.70-5.59 (m, 2H), 5.48 (d, J=7.1 Hz, 1H), 4.19-4.11 (m, 2H), 3.99-3.88 (m, 5H), 3.37 (s, 3H), 2.06-2.03 (m, 3H), 1.22 (dt, J=4.2, 7.0 Hz, 6H)

LCMS (M−H⁺): 403.1, purity: 95.16%

TLC (Ethyl acetate:Methanol=10:1), Rf=0.3

### 9. Preparation of Compound WV-NU-231

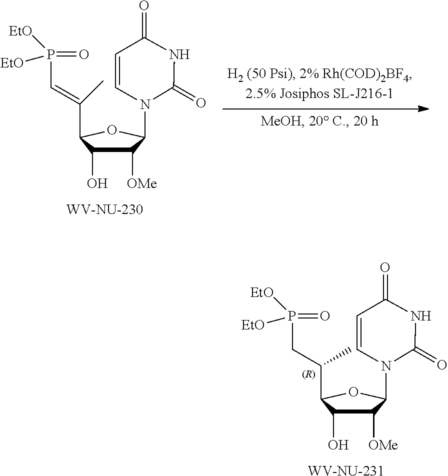

For three batches. To a mixture of compound WV-NU-230 (13 g, 32.15 mmol) in MeOH (200 mL) was added Josiphos SL-J216-1 (1.04 g, 1.62 mmol), (1Z,5Z)-cycloocta-1,5-diene;rhodium(1+);tetrafluoroborate (522.21 mg, 1.29 mmol.) and zinc;trifluoromethanesulfonate (4.68 g, 12.86 mmol). And the system was stirred under H₂ (50 psi) for 20 hr at 20° C. LCMS showed the desired mass was detected. The reaction mixture was filtered and concentrated under reduced pressure to give a residue. The residue was purified by prep-HPLC column: Welch Xtimate C18 250*70 mm #10 um; mobile phase: [water (NH₄HCO₃)-ACN]; B %: 10%-30%, 20 min. Compound WV-NU-231 (28 g, 71.79% yield) was obtained as a white solid. 6.47 g for batch 1(99.68% purity), 21.6 g for batch 2(100% purity).

¹H NMR (400 MHz, DMSO-d₆) δ=11.35 (s, 1H), 7.63 (d, J=8.1 Hz, 1H), 5.73-5.69 (m, 1H), 5.69-5.65 (m, 1H), 4.07-3.92 (m, 5H), 3.82 (t, J=5.6 Hz, 1H), 3.57 (t, J=5.9 Hz, 1H), 3.35-3.32 (m, 3H), 2.07 (s, 1H), 2.02-1.90 (m, 1H), 1.57 (ddd, J=9.8, 15.6, 17.4 Hz, 1H), 1.23 (t, J=7.0 Hz, 6H), 1.03 (d, J=6.6 Hz, 3H)

LCMS (M−H⁺): 405.2; purity: 99.68%

1H NMR (400 MHz, DMSO-d₆) δ=11.39 (br s, 1H), 7.63 (d, J=8.1 Hz, 1H), 5.71 (d, J=5.1 Hz, 1H), 5.67 (d, J=8.1 Hz, 1H), 5.18 (d, J=6.8 Hz, 1H), 4.07-3.93 (m, 5H), 3.82 (t, J=5.5 Hz, 1H), 3.57 (t, J=5.9 Hz, 1H), 3.35-3.34 (m, 3H), 2.07 (s, 1H), 2.04-1.91 (m, 1H), 1.57 (dt, J=9.8, 16.5 Hz, 1H), 1.23 (t, J=7.0 Hz, 6H), 1.03 (d, J=6.6 Hz, 3H) LCMS (M−H⁺): 405.2; purity: 100%

### Example 39: Synthesis of WV-NU-306

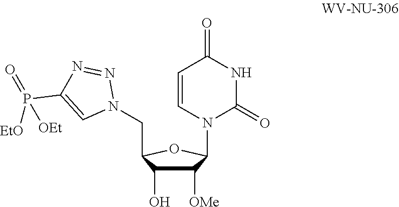

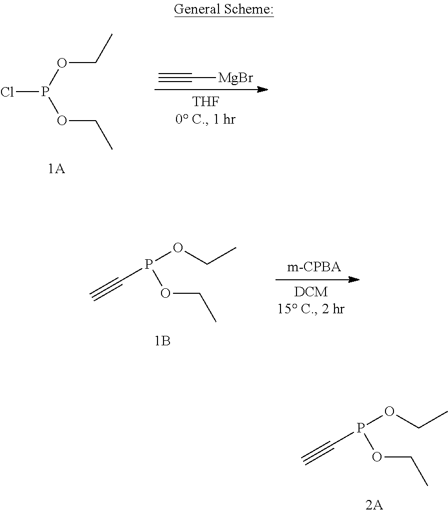

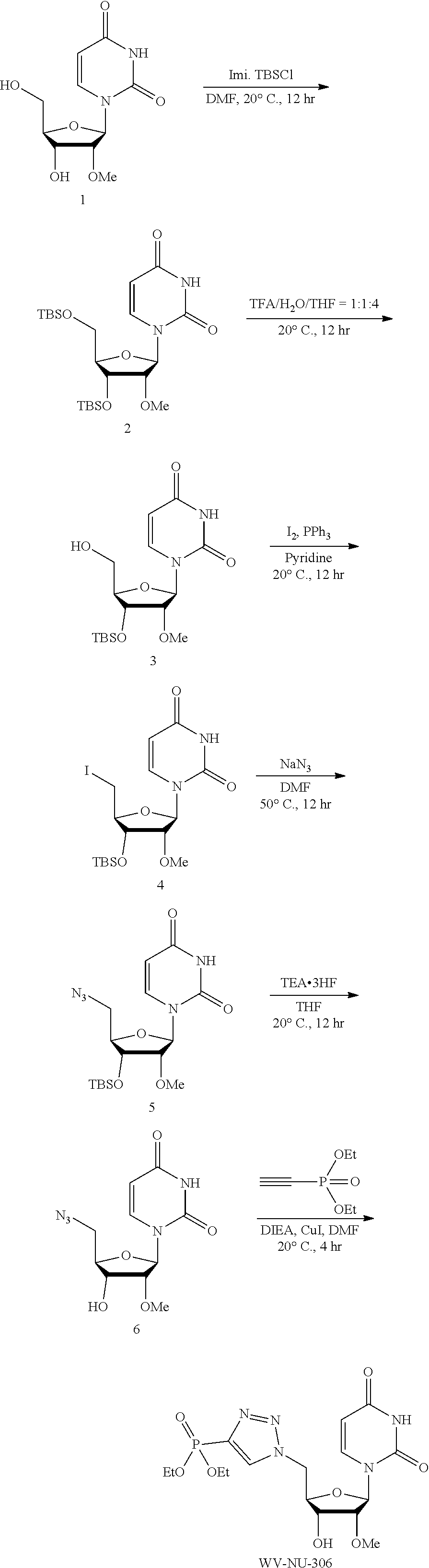

### 1. Preparation of Compound 1B

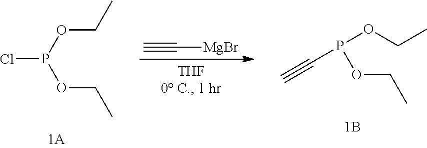

To a solution of compound 1A (20 g, 127.76 mmol) in THF (200 mL) and bromo(ethynyl)magnesium (0.5 M, 258.07 mL) was added at 0° C., and the mixture was stirred at 0° C. for 1 hr. TLC showed compound 1A was consumed completely and two new spots formed. The mixture was quenched by addition sat. NH₄Cl (aq., 50 mL) at 0° C., then diluted with H₂O (200 mL) and extracted with DCM (150 mL*3). The combined organic layers were dried over Na₂SO₄, filtered to get the crude. Compound 1B (18.6 g, crude) in DCM as a yellow liquid was used for next step.

TLC: Petroleum ether:Ethyl acetate=5:1, Rf=0.24

### 2. Preparation of Compound 2A

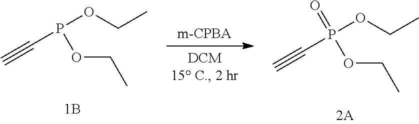

For two batches. To a solution of compound 1B (9 g, 61.59 mmol) in DCM (500 mL) was added m-CPBA (25.01 g, 123.18 mmol, 85% purity). The mixture was stirred at 20° C. for 1 hr. TLC indicated compound 1B was consumed completely and one new spot formed. The reaction was clean according to TLC. Two batches combined with together. The reaction mixture was quenched by sat. aq. Na₂SO₃ (500 mL) and NaHCO₃ (500 mL), then extracted with DCM (100 mL*3). The combined organic layers were washed with brine (100 mL*2), dried over Na₂SO₄, filtered and concentrated under reduced pressure to give a residue. The residue was purified by column chromatography (SiO₂, Petroleum ether/Ethyl acetate=I/O to 0/1). Compound 2A (10 g, 50.07% yield) was obtained as a colorless oily liquid.

¹H NMR (400 MHz, CHLOROFORM-d) 6=4.21-4.13 (m, 4H), 2.94 (d, J=13.3 Hz, 1H), 1.35 (t, J=7.1 Hz, 6H)

TLC: Petroleum ether:Ethyl acetate=1:1, Rf=0.45

### 3. Preparation of Compound 2

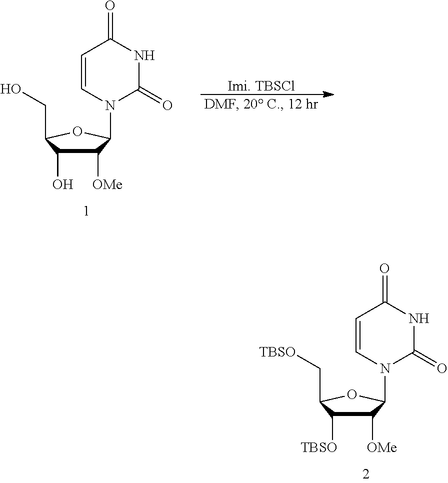

For two batches. To a solution of compound 1 (40 g, 154.90 mmol, 1 eq) in DMF (500 mL) was added imidazole (52.73 g, 774.51 mmol) and TBSCl (93.39 g, 619.61 mmol), the mixture was stirred at 20° C. for 2 hr. TLC indicated compound 1 was consumed completely and one new spot formed. The reaction mixture was concentrated under reduced pressure to remove DMF. The residue was purified by column chromatography (SiO₂, Petroleum ether/Ethyl acetate=1/0 to 0/1). Compound 2 (150 g, 99.47% yield) was obtained as a white solid.

TLC: Petroleum ether:Ethyl acetate=1:1, Rf=0.55

### 4. Preparation of Compound 3

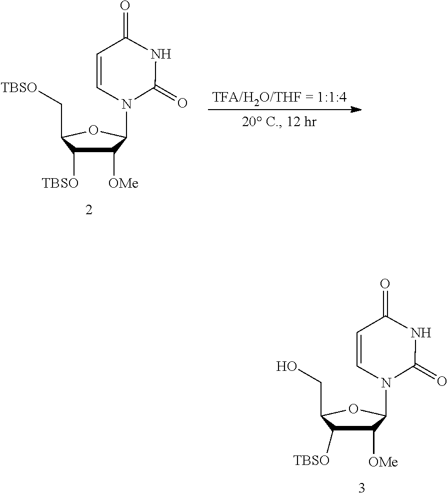

For four batches: To a solution of compound 2 (22.5 g, 46.23 mmol, 1 eq) in THF (350 mL) was added TFA (69.07 g, 605.80 mmol) and H₂O (45.00 g, 2.50 mol). The mixture was stirred at 0° C. for 1 hr. LCMS showed compound 2 was consumed completely and the desired mass was detected. For four batches were combined for workup. The reaction mixture was added NH₃·H₂O (20 ml), then filtered and concentrated under reduced pressure to give a residue. The residue was purified by column chromatography (SiO₂, Petroleum ether/Ethyl acetate=1/0 to 0/1). Compound 3 (55 g, 60.44% yield) was obtained as a white solid.

¹H NMR (400 MHz, CHLOROFORM-d) 6=8.77 (br s, 1H), 7.69 (d, J=8.2 Hz, 1H), 5.74 (dd, J=1.7, 8.0 Hz, 1H), 5.68 (d, J=4.0 Hz, 1H), 4.36 (t, J=5.1 Hz, 1H), 4.10-4.04 (m, 1H), 3.98 (t, J=4.4 Hz, 2H), 3.76 (dd, J=1.7, 12.2 Hz, 1H), 3.50 (s, 4H), 0.92 (s, 10H), 0.12 (d, J=5.4 Hz, 6H)

LCMS (M−H+):371.3

TLC: Petroleum ether:Ethyl acetate=1:1, Rf=0.2

### 5. Preparation of Compound 4

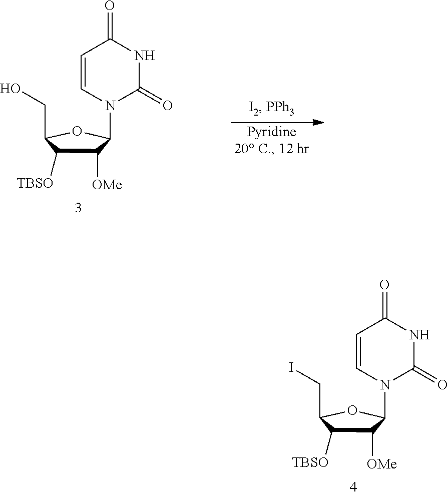

To a solution of compound 3 (50 g, 134.23 mmol) in Py (1000 mL) was added PPh₃ (63.37 g, 241.62 mmol) and I₂ (51.10 g, 201.35 mmol). The mixture was stirred at 25° C. for 12 hr under N₂ atmosphere. LCMS showed compound 3 was consumed completely and the desired mass was detected. The reaction mixture was quenched by sat. aq. Na₂SO₃ (100 mL) and extracted with EtOAc (300 mL*3). The combined organic layers were washed with brine (100 mL*2), dried over Na₂SO₄, filtered and concentrated under reduced pressure to give a residue. The residue was purified by column chromatography (SiO₂, Petroleum ether/Ethyl acetate=I/O to 0/1). Compound 4 (30 g, 42.86% yield) was obtained as a purple solid.

¹H NMR (400 MHz, DMSO-d₆) δ=11.44 (s, 1H), 7.71 (d, J=8.1 Hz, 1H), 5.85 (d, J=5.5 Hz, 1H), 5.71 (dd, J=1.6, 8.1 Hz, 1H), 4.24 (t, J=4.4 Hz, 1H), 4.07 (t, J=5.3 Hz, 1H), 3.87-3.82 (m, 1H), 3.54 (dd, J=6.5, 10.6 Hz, 1H), 3.41-3.36 (m, 1H), 3.31 (s, 3H), 0.89 (s, 9H), 0.14 (d, J=9.2 Hz, 6H)

LCMS (M−H+):483

TLC: Petroleum ether:Ethyl acetate=1:1, Rf=0.6

### 6. Preparation of Compound 5

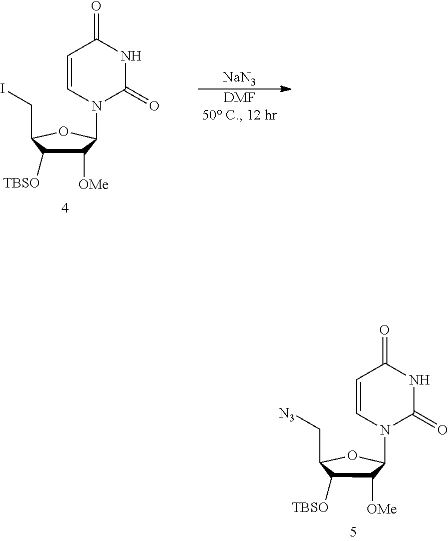

To a solution of compound 4 (24 g, 49.75 mmol) in DMF (120 mL) was added NaN₃ (3.95 g, 60.76 mmol) 0.5 g for 4 portions under N₂. After additional, the mixture was stirred at 50° C. for 12 hr under N₂. LCMS showed compound 4 was consumed completely and the desired mass was detected. The reaction was quenched by H₂O (200 mL), and extracted with Ethyl acetate (300 mL*3). The combined organic layers were washed with saturated aqueous NaCl 150 mL, dried over Na₂SO₄, filtered and concentrated under reduced pressure to give a residue. Compound 5 (19.78 g, crude) was obtained as a red solid.

LCMS: (M+H+):398.1

### 7. Preparation of Compound 6

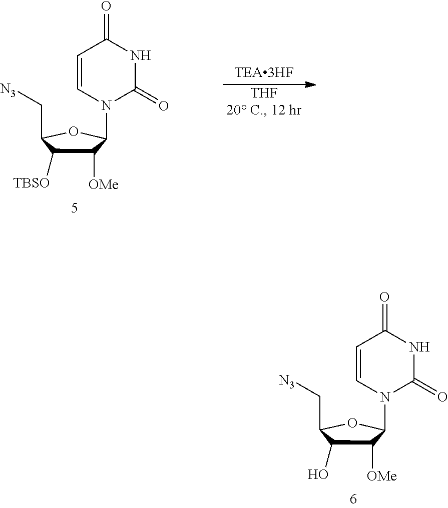

To a solution of compound 5 (19.78 g, 49.76 mmol) in THF (200 mL) was added N,N— diethylethanamine;trihydrofluoride (32.09 g, 199.04 mmol). The mixture was stirred at 20° C. for 12 hr. LCMS showed compound 5 was consumed completely and the desired mass was detected. The reaction mixture was neutralized with sat.Na₂CO₃ (aq.) until pH=7. The mixture was concentrated under reduced pressure to removed most of water. The mixture was added DCM (40 mL) and dried over Na₂SO₄, filtered and concentrated under reduced pressure to give a residue. The residue was purified by column chromatography (SiO₂, Petroleum ether/Ethyl acetate=1/0 to 0/1). Compound 6 (11 g, 8.07% yield) was obtained as a yellow solid.

¹H NMR (400 MHz, DMSO-d₆) δ=11.41 (s, 1H), 7.70 (d, J=8.1 Hz, 1H), 5.83 (d, J=4.9 Hz, 1H), 5.68 (dd, J=1.9, 8.1 Hz, 1H), 5.36 (d, J=6.3 Hz, 1H), 3.96-3.87 (m, 2H), 3.61 (d, J=4.9 Hz, 2H), 3.36 (s, 3H)

LCMS (M−H+):284.1

TLC: Petroleum ether:Ethyl acetate=0:1, R_{f}=0.4

### 8. Preparation of WV-NU-306

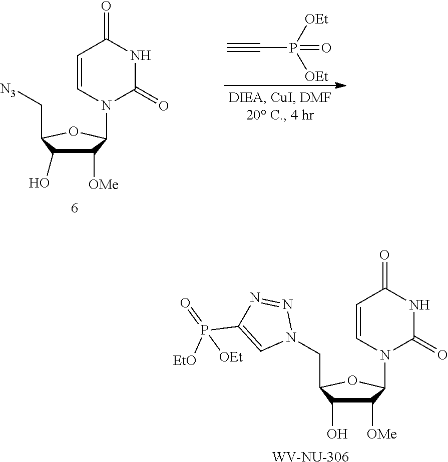

For three batches. To a solution of compound 6 (4 g, 14.12 mmol) and compound 2A (2.68 g, 16.52 mmol) in DMF (40 mL) was degassed and purged with N₂ for 3 times, then DIEA (3.65 g, 28.24 mmol), CuI (5.38 g, 28.24 mmol) was added. The mixture was stirred at 20° C. for 4 hr under N₂ atmosphere. LCMS showed compound 6 was consumed completely and the desired mass was detected. Three batches combined with together. The reaction mixture was concentrated under reduced pressure to give product. The residue was purified by column chromatography (SiO₂, DCM: Methanol=1/0 to 0/1). WV-NU-306 (16 g, 84.79% yield) was obtained as a yellow solid.

¹H NMR (400 MHz, CHLOROFORM-d) 6=9.78 (s, 1H), 8.28 (s, 1H), 7.03 (d, J=8.1 Hz, 1H), 5.73 (dd, J=1.6, 8.1 Hz, 1H), 5.61 (d, J=2.3 Hz, 1H), 4.96-4.88 (m, 1H), 4.75 (dd, J=5.7, 14.4 Hz, 1H), 4.28-4.15 (m, 6H), 3.97 (dd, J=2.3, 5.0 Hz, 1H), 3.66 (br d, J=6.8 Hz, 1H), 3.55 (s, 3H), 1.35 (t, J=7.0 Hz, 6H)

³¹P NMR (162 MHz, CHLOROFORM-d) δ=6.72 (s, 1P)

LCMS (M−H⁺):446, LCMS purity: 94.74%

TLC: DCM:MeOH=10:1, Rf=0.65

### Example 40: Synthesis of WV-NU-299

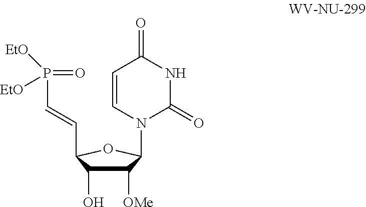

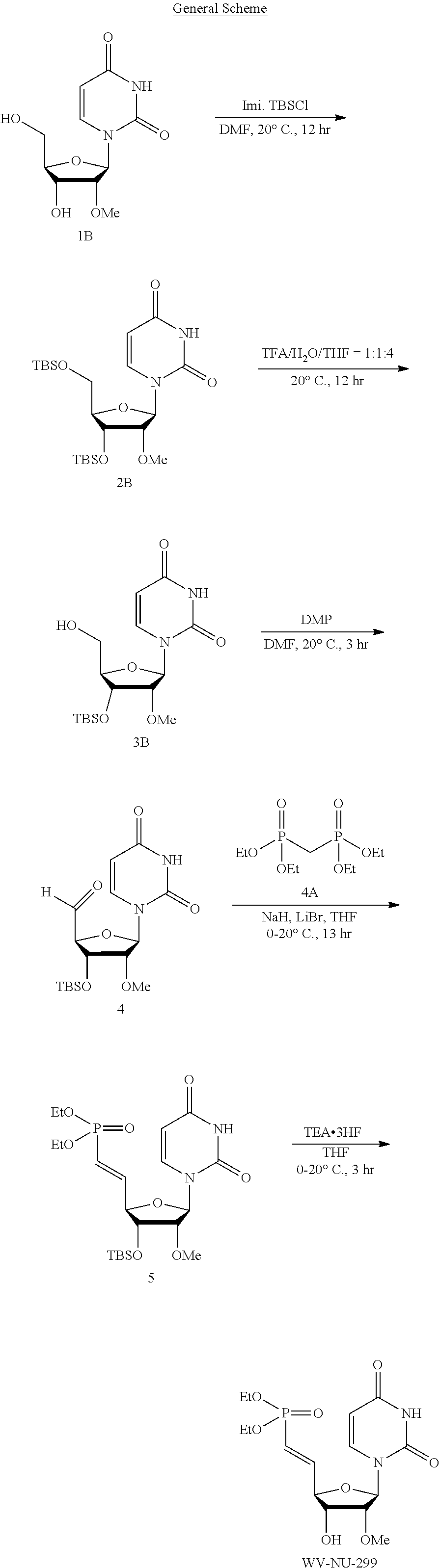

### 1. Preparation of Compound 2B

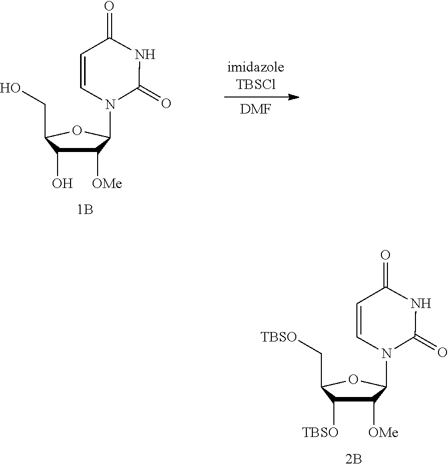

To a solution of compound 1B (50 g, 193.63 mmol) in DMF (800 mL) was added imidazole (65.91 g, 968.14 mmol) and TBSCl (116.74 g, 774.51 mmol). The mixture was stirred at 20° C. for 2 hr. LCMS showed compound 1B was consumed completely and one main peak with desired mass was detected. The reaction mixture was concentrated under reduced pressure to remove DMF. The residue was purified by column chromatography (SiO₂, Petroleum ether/Ethyl acetate=1/0 to 0/1). Compound 2B (180 g, 95.74% yield) was obtained as a white solid.

¹HNMR (400 MHz, CHLOROFORM-d) 6=9.35-9.27 (m, 1H), 8.05 (d, J=8.1 Hz, 1H), 5.93 (d, J=1.5 Hz, 1H), 5.67 (d, J=8.0 Hz, 1H), 4.23 (dd, J=4.9, 7.1 Hz, 1H), 4.06-4.01 (m, 2H), 3.77 (d, J=10.4 Hz, 1H), 3.59 (dd, J=1.6, 4.8 Hz, 1H), 3.55 (s, 3H), 0.91 (s, 9H), 0.90 (s, 9H), 0.09 (t, J=3.6 Hz, 12H)

TLC (Ethyl acetate:Methanol=3:1), R_{f}=0.3

LCMS (M−H⁺): 485.4

### 2. Preparation of Compound 3B

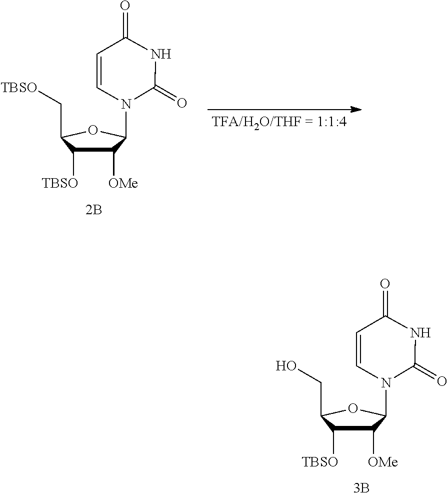

For two batches: To a solution of compound 2B (94 g, 193.12 mmol, 1 eq) in THF (800 mL) was added the mixture of TFA (200 mL) and H₂O (200 mL). The mixture was stirred at 0° C. for 3 hr. LCMS showed compound 2B was consumed completely and one main peak with desired mass was detected. The reaction mixture was concentrated under reduced pressure to remove solvent. The residue was purified by column chromatography (SiO2, Petroleum ether/Ethyl acetate=1/0 to 0/1). Compound 3B (74 g, 52.86% yield) was obtained as a white solid.

¹HNMR (400 MHz, CHLOROFORM-d) 6=9.55-9.48 (m, 1H), 7.74 (d, J=8.1 Hz, 1H), 5.77-5.69 (m, 2H), 4.35 (t, J=5.3 Hz, 1H), 4.09-4.04 (m, 1H), 4.02-3.93 (m, 2H), 3.79-3.72 (m, 1H), 3.49 (s, 3H), 2.74 (br s, 1H), 0.91 (s, 9H), 0.11 (d, J=5.0 Hz, 6H)

LCMS (M−H⁺): 371.1

TLC (Petroleum ether:Ethyl acetate=1:1), Rf=0.5

### 3. Preparation of Compound 4

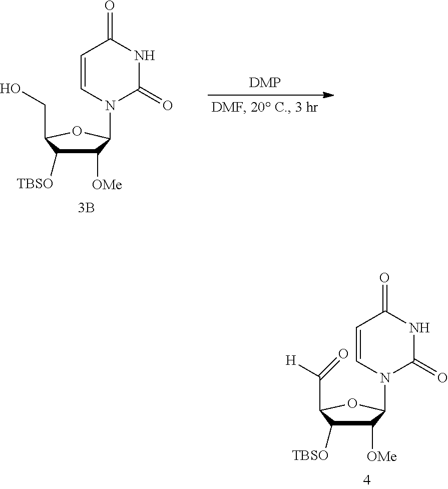

For two batches: To a solution of compound 3B (25 g, 67.12 mmol) in DCM (1400 mL) was added DMP (42.70 g, 100.67 mmol) at 0° C. The mixture was stirred at 20° C. for 3 hr. TLC indicated compound 3B was consumed completely and one new spot formed. The reaction mixture of two batches were diluted with Na₂SO₃: NaHCO₃=1:2 700 mL and extracted with DCM 60*3 mL. The combined organic layers were washed with Sat. NaCl 800 mL, dried over Na₂SO₄, filtered and concentrated under reduced pressure to get compound 4 (49.7 g, crude) was obtained as a yellow oil.

TLC (Petroleum ether:Ethyl acetate=1:1), Rf=0.18

### 4. Preparation of Compound 5

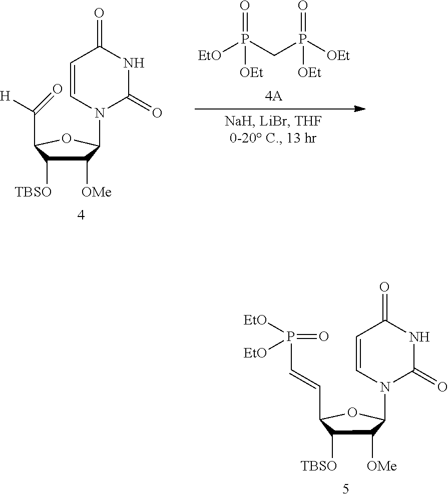

To a solution of NaH (11.78 g, 294.54 mmol, 60% purity) in THF (120 mL) was added compound 4A (84.89 g, 294.54 mmol) in THF (800 mL) at 0° C. The reaction mixture was warmed up to 20° C., and stirred for 1 hr. A solution of LiBr (25.58 g, 294.54 mmol) in THF (250 mL) was added and the resultant slurry was stirred, and then cooled to 0° C. To the above mixture was added a solution of compound 4 (24.8 g, 66.94 mmol) in THF (250 mL) at 0° C. The mixture was stirred at 0-20° C. for 12 hr. LCMS showed compound 4 was consumed completely and one main peak with desired mass was detected. The reaction mixture was diluted with H2O 3000 mL and extracted with EtOAc 500*3 mL. The combined organic layers were washed with Sat. NaCl 1000 mL, dried over Na₂SO₄, filtered and concentrated under reduced pressure to give a residue. The residue was purified by column chromatography (SiO₂, Petroleum ether/Ethyl acetate=I/O to 0/1). Compound 5 (31.2 g, 46.57% yield) was obtained as a white solid.

¹H NMR (400 MHz, DMSO-d₆) δ=11.41 (s, 1H), 7.70 (d, J=8.1 Hz, 1H), 6.75-6.62 (m, 1H), 6.14-6.01 (m, 1H), 5.77 (d, J=3.1 Hz, 1H), 5.67 (d, J=8.1 Hz, 1H), 4.36-4.26 (m, 2H), 3.99-3.93 (m, 5H), 3.37 (s, 3H), 1.23 (s, 12H), 0.87 (s, 9H)

LCMS (M+H⁺): 505.4

TLC (Petroleum ether:Ethyl acetate=3:1), Rf=0.4

### 5. Preparation of Compound WV-NU-299

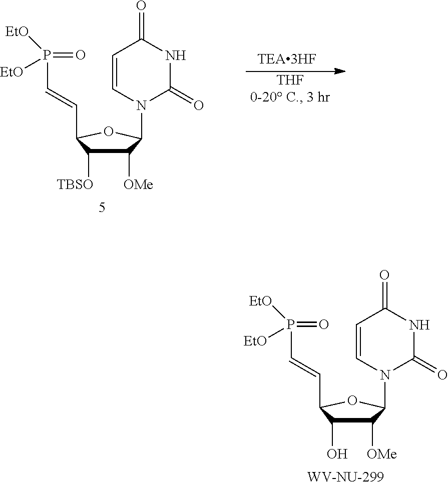

To a solution of compound 5 (34 g, 67.38 mmol) in THF (340 mL) was added N,N-diethylethanamine;trihydrofluoride (43.45 g, 269.53 mmol). The mixture was stirred at 40° C. for 6 hr. TLC indicated compound 5 was consumed completely and one new spot formed. The reaction mixture was concentrated under reduced pressure to remove DMF. The residue was purified by column chromatography (SiO₂, DCM:MeOH=1:0 to 0:1) to get WV-NU-299 (11.3 g, 42.96% yield) as a white solid.

¹H NMR (400 MHz, DMSO-d₆) δ=11.41 (s, 1H), 7.63 (d, J=8.1 Hz, 1H), 6.80-6.66 (m, 1H), 6.07-5.96 (m, 1H), 5.81 (d, J=4.1 Hz, 1H), 5.66 (d, J=8.0 Hz, 1H), 5.50 (d, J=6.6 Hz, 1H), 4.38-4.31 (m, 1H), 4.04-3.91 (m, 6H), 3.38 (s, 3H), 1.23 (dt, J=1.4, 7.1 Hz, 6H)

LCMS (M−H⁺): 389.0

TLC (DCM:MeOH=10:1, R_{f}=0.25)

### Example 41: Synthesis of (WV-NU-301)

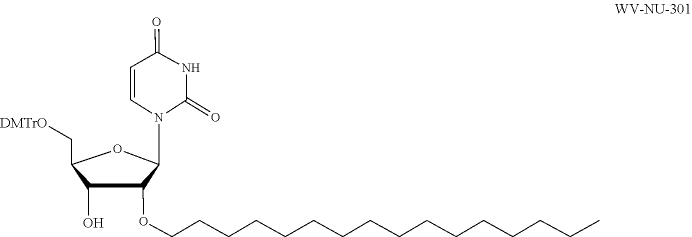

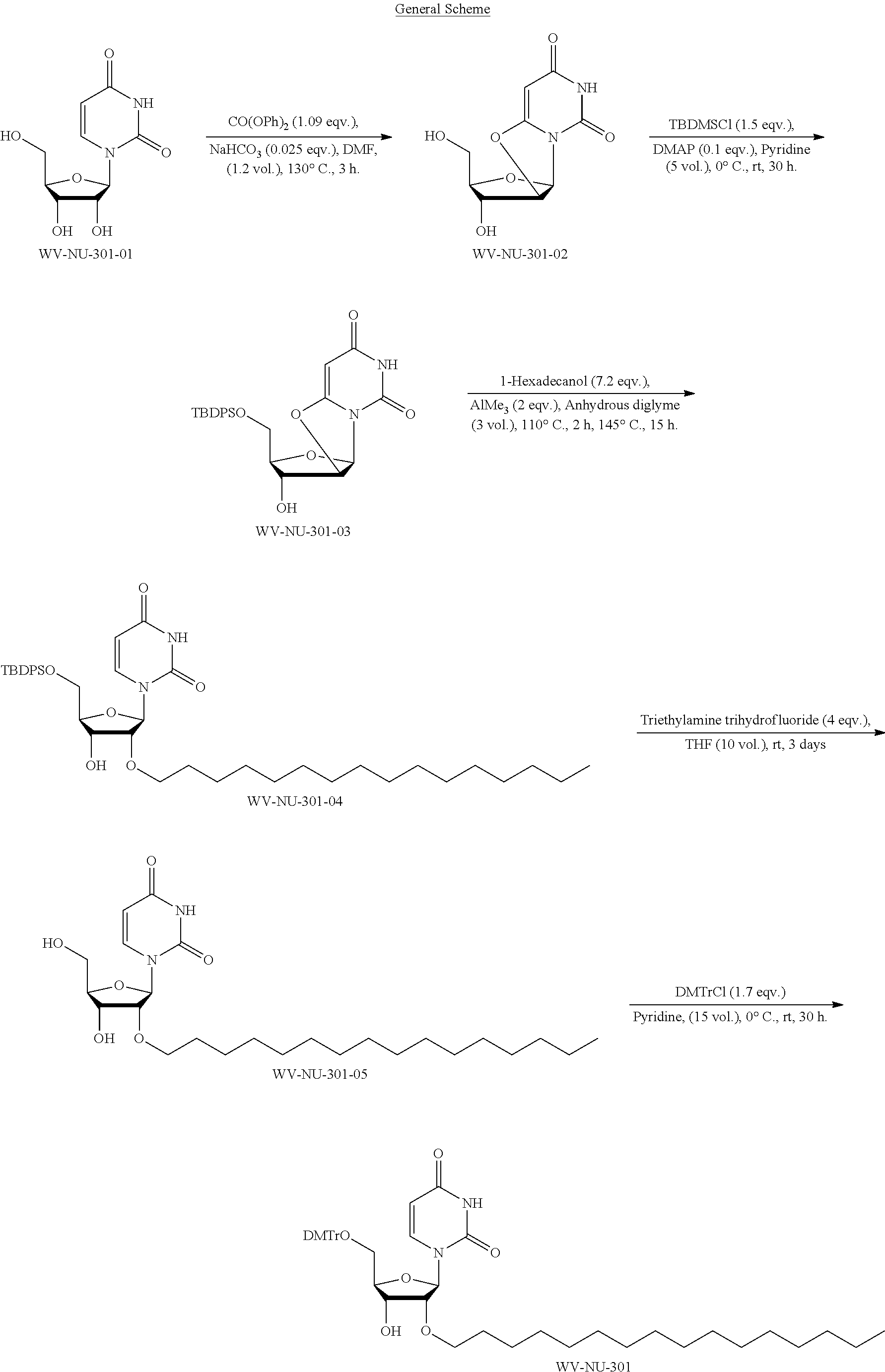

### 1. Preparation of (2R,3R,3aS,9aR)-3-hydroxy-2-(hydroxymethyl)-2,3,3a,9a-tetrahydro-6H-furo[2′,3′:4,5]oxazolo[3,2-c]pyrimidine-6,8(7H)-dione (WV-NU-302-02)

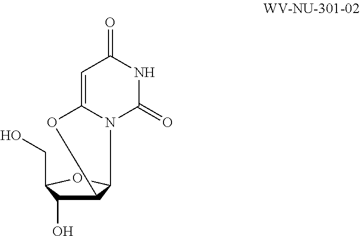

To a stirred solution of Uridine (50 g, 0.2049 mol) and diphenyl carbonate (47.79 g, 0.2233 mol.) in dry DMF (60 mL, 1.2 vol.) was added sodium bicarbonate (430 mg, 0.00512 mol) stirred at 130° C. for 3 h. Progress of the reaction was monitored by TLC. Then reaction mixture was cooled to room temperature, precipitated product was observed, filtered and washed with cool methanol (2×20 mL), dried under vacuum to get as off white solid (WV—NU-301-02) (35 g, 70%). TLC Mobile phase details: 15% MeOH in DCM.

¹H NMR (400 MHz, DMSO-d₆): δ in ppm=7.82 (dd, 1H, J1=7.4 Hz, J2=0.8 Hz), 6.30 (d, 1H, J1=5.8 Hz), 5.85 (dd, 1H, J1=7.4 Hz, J2=0.4 Hz), 5.19 (d, 1H, J1=5.6 Hz), 4.37 (s, 1H), 4.07 (dd, 1H, J1=5.3 Hz, J2=1.5 Hz), 3.27 (dd, 1H, J1=11.6 Hz, J2=5.0 Hz), 3.18 (q, 3H, J1=5.8 Hz).

MS: m z calcd for C₉H₁₀N₂O₆, 242.2; found 242.3. [M⁺].

### 2. Preparation of (2R,3R,3aS,9aR)-2-(((tert-butyldiphenylsilyl)oxy)methyl)-3-hydroxy-2,3,3a,9a-tetrahydro-6H-furo[2′,3′:4,5]oxazolo[3,2-c]pyrimidine-6,8(7H)-dione (WV—NU-301-03)

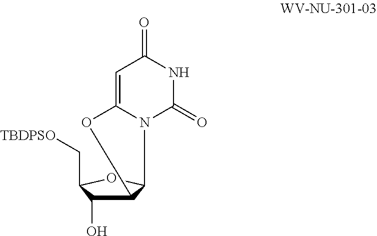

To a stirred solution of (WV-NU-301-02) (30 g, 0.1239 mol) and DMAP (1.38 g, 0.0123 mol.) in anhydrous pyridine (150 mL, 5 vol.) was added TBDPSCl (47.2 mL, 0.1859 mol.) dropwise over a period of 30 mins, at 0° C. Above reaction mixture was stirred at rt for 30 h. Progress of the reaction was monitored by TLC. The reaction was diluted with cold sat. NaHCO₃ (150 mL) and extracted with DCM (2×200 mL), washed with brine (1×100 mL) solution (1×100 mL), dried over Na₂SO₄ and concentrated under reduced pressure. The crude compound was purified by column chromatography over silica-gel (230-400 mesh) eluted in 3% MeOH/DCM to afford an off-white solid. (WV-NU-301-03) (27 g, 46%). TLC Mobile phase details: 10% MeOH in DCM.

¹H NMR (400 MHz, DMSO-d₆): δ in ppm=7.92 (d, 1H, J1=7.4 Hz), 7.53 (m, 4H), 7.43 (m, 6H), 6.32 (d, 1H, J1=5.8 Hz), 6.01 (d, 1H, J1=4.7 Hz), 5.87 (d, 1H, J1=7.4 Hz), 5.26 (dd, 1H, J1=5.6 Hz), 4.44 (t, 1H, J1=3.2 Hz), 3.59 (dd, 1H, J1=11.3 Hz, J2=4.7 Hz), 3.47 (dd, 1H, J1=11.3 Hz, J2=6.5 Hz), 0.92 (s, 9H), 7.53 (m, 4H).

### 3. Preparation of 1-((2R,3R,4R,5R)-3-(hexadecyloxy)-4-hydroxy-5-(hydroxymethyl)tetrahydrofuran-2-yl)pyrimidine-2,4(1H,3H)-dione (WV-NU-301-05)

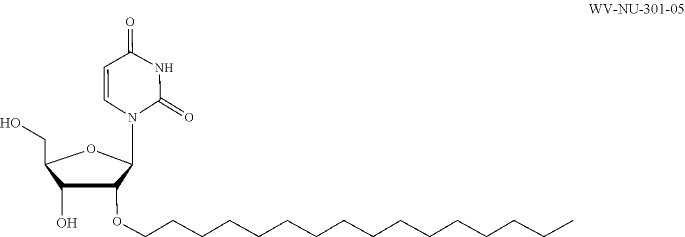

To a stirred solution of 1-Hexadecanol (108.9 g, 0.451 mol)) in anhydrous diglyme (84 L, 2.8 vol.) was added Trimethylaluminum (71.1 mL, 0.150 mol, 2M solution in toluene) dropwise over a period of 40 min. The resulting mixture was heated to 120° C. and stirred for 2 h. Then the mass was allowed to rt and (WV-NU-301-03) (30 g, 0.0625 mol) was added, stirred at 145° C. for 15 h. Progress of the reaction was monitored by TLC. The reaction mixture was diluted 10% H₃PO₄ (500 mL) and EtOAc (300 mL), organic layer was separated washed with 5% NaCl (100 mL), dried over Na₂SO₄ and concentrated under vacuum to afford as a gummy syrup (56 g, crude). The syrup was dissolved in THF (300 mL, 10 vol.), was added triethyl amine hydrofluoride (40.7 mL, 0.250 mol.), stirred rt for 3 days. Progress of the reaction was monitored by TLC. The reaction was diluted 5% NaCl (300 mL) and EtOAc (300 mL), organic layer was separated washed with 5% NaCl (100 mL) washed with sat. NaCl solution (1×100 mL), dried over Na₂SO₄ and concentrated under reduced pressure. The crude compound was purified by column chromatography over silica-gel (230-400 mesh) eluted in 3% MeOH in DCM to afford an off-white solid. (WV—NU-301-05) (12 g, 40%). TLC Mobile phase details: 10% MeOH in DCM.

¹H NMR (400 MHz, DMSO-d₆): δ in ppm=11.33 (s, 1H), 7.94 (d, 1H, J1=8.2 Hz), 5.83 (d, 1H, J1=5.1 Hz), 5.64 (dd, 1H, J1=8.1 Hz, J2=1.6 Hz), 5.14 (d, 1H, J1=5.1 Hz), 5.04 (d, 1H, J1=5.8 Hz), 4.09 (dt, 2H, J1=7.6 Hz, J1=2.6 Hz), 3.84 (m, 2H), 3.64 (m, 1H), 3.55 (m, 2H), 3.46 (m, 2H), 3.23 (s, 1H), 3.17 (d, 2H, J1=5.2 Hz), 1.43 (m, 3H), 1.23 (s, 34H), 0.85 (t, 4H, J1=6.8 Hz), MS: m czalcd for C₂₅H₄₄N₂O₆, 468.6; found 467.3 (M−H⁺

### 4. Preparation of 1-((2R,3R,4R,5R)-5-((bis(4-methoxyphenyl)(phenyl)methoxy)methyl)-3-(hexadecyloxy)-4-hydroxytetrahydrofuran-2-yl)pyrimidine-2,4(1H,3H)-dione (WV—NU-301)

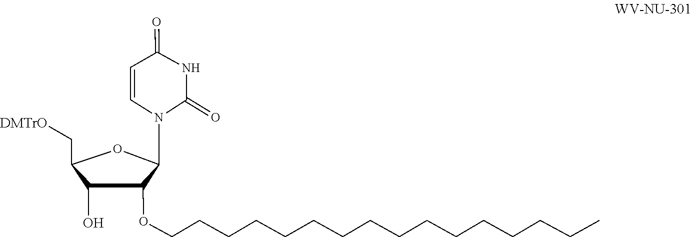

To a stirred solution of (WV-NU-301-05) (6 g, 0.01282 mol) in anhydrous Pyridine (90 mL, 15 vol.) was added DMTCl (7.3 g, 0.02179 mol) portion-wise over a period of 15 min at 0° C. Above reaction was stirred at rt for 30 h. Progress of the reaction was monitored by TLC. Then reaction was concentrated under vacuum to get crude mass. The crude was dissolved in ethyl acetate (100 mL), washed with sat. NaHCO₃ (30 mL×2), brine solution (30 mL×1), dried over Na₂SO₄, concentrated and purified by column chromatography over silica gel (230-400 mesh) eluted in 35% EtOAc/Hexane to get as an off white solid (WV—NU-313) (7 g, 70%). TLC Mobile phase details: 50% EtOAc in Hexane.

¹H NMR (400 MHz, DMSO-d₆): δ in ppm=11.37 (s, 1H), 7.72 (d, 1H, J1=8.1 Hz), 7.38 (m, 1H), 7.32 (m, 2H), 7.24 (m, 5H), 6.90 (d, 4H, J1=8.8 Hz), 5.80 (d, 1H, J1=3.8 Hz), 5.28 (d, 1H, J1=8.0 Hz), 5.12 (d, 1H, J1=6.6 Hz), 4.17 (dd, 1H, J1=11.6 Hz, J1=6.1 Hz), 4.96 (m, 1H), 3.90 (t, 1H, J1=4.5 Hz), 3.74 (s, 6H), 3.56 (m, 2H), 3.25 (m, 2H), 1.49 (q, 2H, J1=6.6 Hz), 2.14 (d, 28H, J1=15.0 Hz), 0.85 (t, 3H, J1=6.9 Hz),

MS: m czalcd for C₄₆H₆₂N₂O₈, 771.0; found 770.01 (M−H⁺).

### Example 42: Synthesis of L- and D-DPSE-2′-OMe-5′-Phosphonate Uridine Amidites

### General Procedure for Preparation of L- and D-DPSE-2′-OMe-5′-Phosphonate Uridine Amidites

Nucleosides (1.0 eq.) in an appropriate size three necked flask was azeotrope with anhydrous toluene (15 mL/g) and anhydrous acetonitrile (15 mL/g) and was dried for 24 hours on high vacuum. To the flask was added anhydrous THF (0.2 M solution) under argon and solution was cooled to −5° C. To the reaction mixture was added triethyl amine (5.0 eq.) followed by addition of D-DPSE-Cl (1.25 M) or L-DPSE-Cl (0.9M) solution (1.8-2.2 eq.) over the period of 5-10 min. The reaction mixture was warmed to room temperature and reaction progress was monitored by HPLC. After disappearance of starting material, the reaction mixture was filtered through fritted glass tube. Reaction flask and precipitate was washed with anhydrous THF. Obtained filtrate was collected and solvent was removed under reduced pressure. The residue was purified by column chromatography (SiO2, 40-100% Ethyl acetate in Hexanes with 5% triethyl amine) to give the corresponding D-DPSE and/or L-DPSE Amidites as off-white solid.

### Synthesis of L-DPSE-2′-OMe-5′-(R)-Me-PO(OEt)₂-Uridine amidite

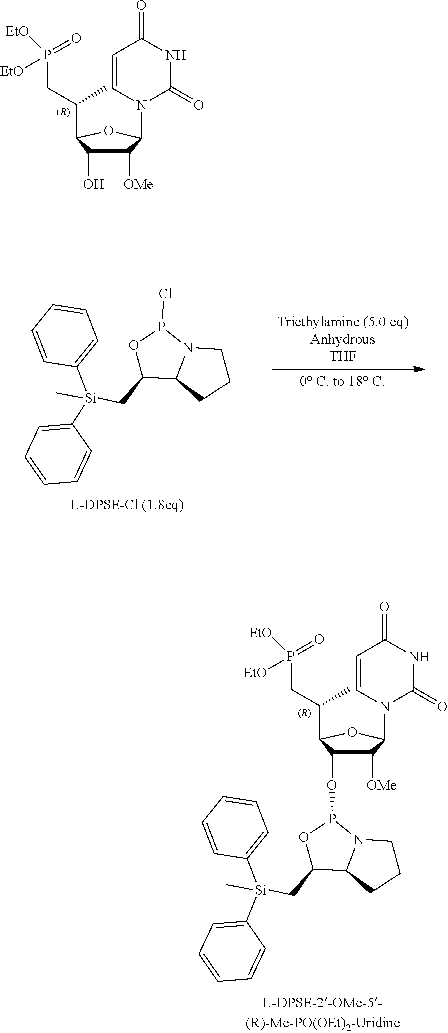

Nucleoside, 2′-OMe′-5′-(R)-Me-PO(OEt)₂-U (WV-NU-231, 5.0 g) was converted to L-DPSE-2′-OMe-5′-(R)-Me-PO(OEt)₂-Uridine amidite by general procedure (7.9 g g, 84% yield) as an off-white solid.

LCMS: C₃₅H₄₉N₃O₉P₂Si (M−H⁻). 744.85

¹H NMR (600 MHz, CDCl₃) δ ¹H NMR (600 MHz, CDCl₃) δ 8.48 (s, 1H), 7.64-7.47 (m, 5H), 7.38 (ddt, J=16.6, 8.8, 4.8 Hz, 5H), 7.27 (d, J=8.1 Hz, 1H), 5.77 (d, J=8.1 Hz, 1H), 5.72 (d, J=3.2 Hz, 1H), 4.95 (q, J=7.1 Hz, 1H), 4.21 (dt, J=9.7, 6.5 Hz, 1H), 4.18-4.04 (m, 3H), 3.89 (t, J=6.4 Hz, 1H), 3.69 (dd, J=5.7, 3.2 Hz, 1H), 3.57 (ddt, J=14.8, 10.5, 7.5 Hz, 1H), 3.46-3.39 (m, 1H), 3.27 (s, 3H), 3.18 (tdt, J=15.2, 10.6, 5.3 Hz, 1H), 2.34-2.22 (m, 1H), 2.10-1.97 (m, 2H), 1.85 (dtt, J=12.2, 8.1, 3.3 Hz, 1H), 1.69 (pd, J=16.4, 8.5 Hz, 4H), 1.51 (dd, J=14.5, 7.8 Hz, 1H), 1.34 (td, J=7.0, 2.2 Hz, 6H), 1.31-1.22 (m, 2H), 1.17 (d, J=6.8 Hz, 3H), 0.67 (s. 3H).

³¹P NMR (243 MHz, CDCl₃) δ=155.81, 30.61

¹³C NMR (151 MHz, CDCl₃) δ 162.64, 149.63, 140.01, 136.33, 136.11, 134.55, 134.51, 134.48, 134.46, 134.42, 129.58, 129.53, 128.09, 128.01, 127.98, 127.87, 102.66, 88.98, 85.60, 85.57, 85.45, 82.70, 79.07, 79.01, 71.21, 71.11, 67.33, 67.31, 61.60, 61.55, 61.51, 58.45, 46.86, 46.63, 30.53, 30.50, 29.72, 28.78, 26.96, 25.97, 25.95, 18.03, 18.01, 16.52, 16.51, 16.48, 16.46, 15.85, 15.82, −3.40.

### Synthesis of D-DPSE-2′-OMe-5′-(R)-Me-PO(OEt)₂-Uridine amidite

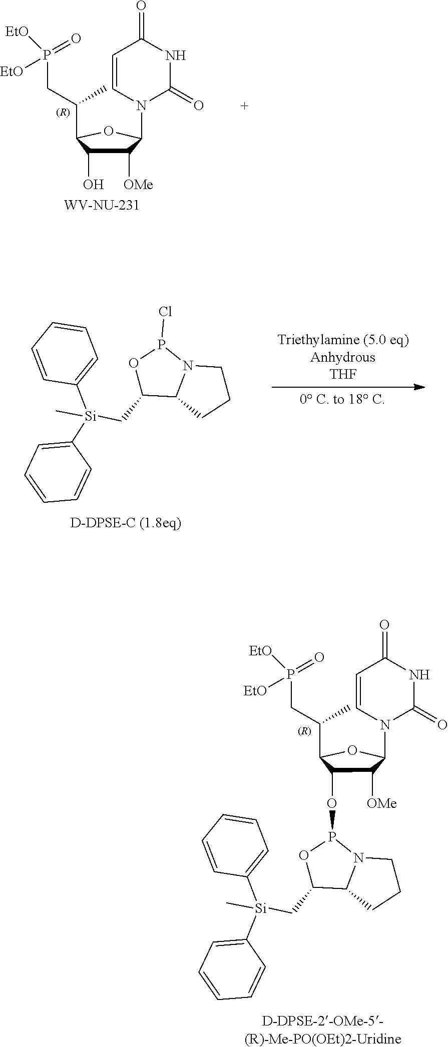

Nucleoside, 2‘—OMe’-5′-(R)-Me-PO(OEt)₂-U (WV-NU-231,2.5 g) was converted to D-DPSE-2′-OMe-5′-(R)-Me-PO(OEt)₂-Uridine amidite by general procedure (2.8 g g, 83% yield) as an off-white solid.

LCMS: C₃₅H₄₉N₃O₉P₂Si (M−H⁻). 744.85

¹H NMR (600 MHz, CDCl₃) δ 9.24 (s, 1H), 7.54 (td, J=7.4, 1.7 Hz, 5H), 7.42-7.32 (m, 5H), 7.27 (d, J=8.1 Hz, 1H), 5.77 (d, J=8.1 Hz, 1H), 5.73 (d, J=3.1 Hz, 1H), 4.94 (td, J=7.5, 5.3 Hz, 1H), 4.21 (ddd, J=9.7, 7.2, 5.6 Hz, 1H), 4.11 (qdd, J=15.1, 6.9, 4.1 Hz, 4H), 3.88 (dd, J=7.3, 5.5 Hz, 1H), 3.69 (dd, J=5.7, 3.1 Hz, 1H), 3.56 (ddt, J=14.7, 10.6, 7.6 Hz, 1H), 3.41 (ddd, J=12.3, 9.8, 5.5 Hz, 1H), 3.27 (s, 3H), 3.18 (tdd, J=10.9, 8.8, 4.5 Hz, 1H), 2.29 (ttd, J=8.8, 6.4, 3.0 Hz, 1H), 2.06-1.97 (m, 1H), 1.84 (dp, J=12.7, 4.3 Hz, 1H), 1.68 (td, J=15.5, 7.5 Hz, 3H), 1.51 (dd, J=14.5, 7.8 Hz, 1H), 1.33 (td, J=7.0, 1.8 Hz, 6H), 1.29-1.24 (m, 1H), 1.17 (d, J=6.7 Hz, 3H), 0.68 (s, 3H).

³¹P NMR (243 MHz, CDCl₃) δ=155.73, 30.66

¹³C NMR (151 MHz, CDCl₃) δ 163.22, 149.88, 139.98, 136.34, 136.11, 134.55, 134.51, 134.49, 134.45, 129.57, 129.52, 128.00, 127.97, 127.85, 102.68, 88.93, 82.73, 79.06, 79.00, 71.24, 71.14, 67.32, 67.30, 61.60, 61.56, 61.51, 58.45, 46.85, 46.62, 30.52, 30.50, 29.69, 28.75, 26.96, 25.97, 25.95, 18.03, 18.01, 16.52, 16.50, 16.48, 16.46, 15.86, 15.84, −3.40.

### Synthesis of L-DPSE-2′-OMe-5′-Me-PO(OEt)₂-Uridine amidite

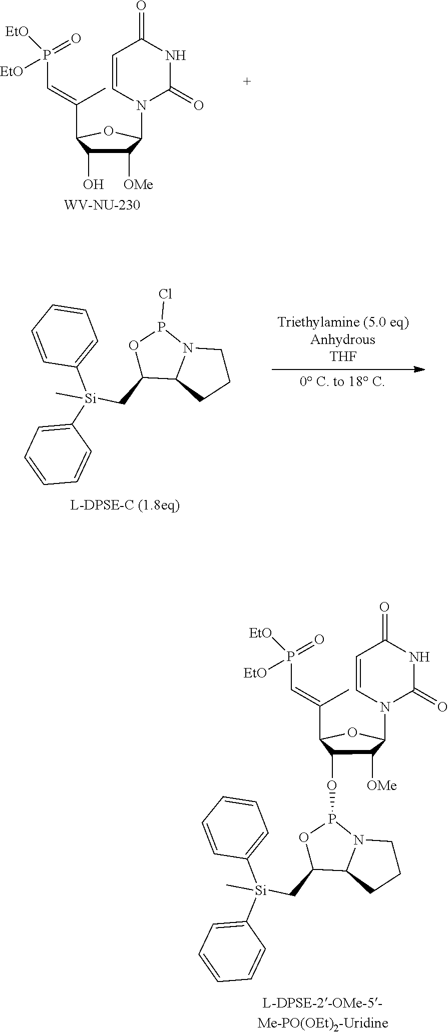

Nucleoside, 2‘—OMe’-5′-Me-PO(OEt)₂-U (WV-NU-230,1.6 g) was converted to L-DPSE-2′-OMe-5′-Me-PO(OEt)₂-Uridine amidite by general procedure (1.98 g g, 67% yield) as an off-white solid.

LCMS: C₃₅H₄₇N₃O₉P₂Si (M−H⁻). 742.69

³¹P NMR (243 MHz, CDCl₃) δ=153.09, 17.24

¹H NMR (600 MHz, CDCl₃) δ=9.20 (s, 1H), 7.47-7.35 (m, 5H), 7.24 (q, J=6.2 Hz, 7H), 7.07 (d, J=8.1 Hz, 1H), 5.71-5.61 (m, 2H), 5.56 (t, J=2.7 Hz, 1H), 4.80 (q, J=6.8 Hz, 1H), 4.32 (dt, J=9.4, 6.1 Hz, 1H), 4.11 (d, J=6.3 Hz, 1H), 3.98 (tt, J=12.8, 7.9 Hz, 4H), 3.63 (t, J=4.8 Hz, 1H), 3.47-3.39 (m, 1H), 3.37-3.32 (m, 1H), 3.28 (d, J=2.0 Hz, 3H), 3.08 (qd, J=10.4, 4.2 Hz, 1H), 1.95 (d, J=3.2 Hz, 3H), 1.75 (dp, J=12.9, 5.1 Hz, 1H), 1.62-1.52 (m, 2H), 1.37 (dd, J=14.6, 6.6 Hz, 1H), 1.32-1.27 (m, 1H), 1.22 (t, J=6.9 Hz, 6H), 1.15 (td, J=8.6, 2.4 Hz, 1H), 0.55 (s, 3H).

¹³C NMR (151 MHz, CDCl₃) δ=163.12, 156.31, 156.26, 149.74, 141.09, 136.48, 135.94, 134.53, 134.51, 134.48, 134.36, 129.54, 129.47, 129.27, 128.14, 128.00, 127.96, 127.86, 113.72, 112.46, 102.90, 90.63, 85.36, 85.34, 85.21, 85.19, 81.33, 81.31, 79.55, 79.49, 77.27, 77.06,

### Synthesis of L-DPSE-2′-OMe-5′-Triazole-PO(OEt)₂-Uridine amidite

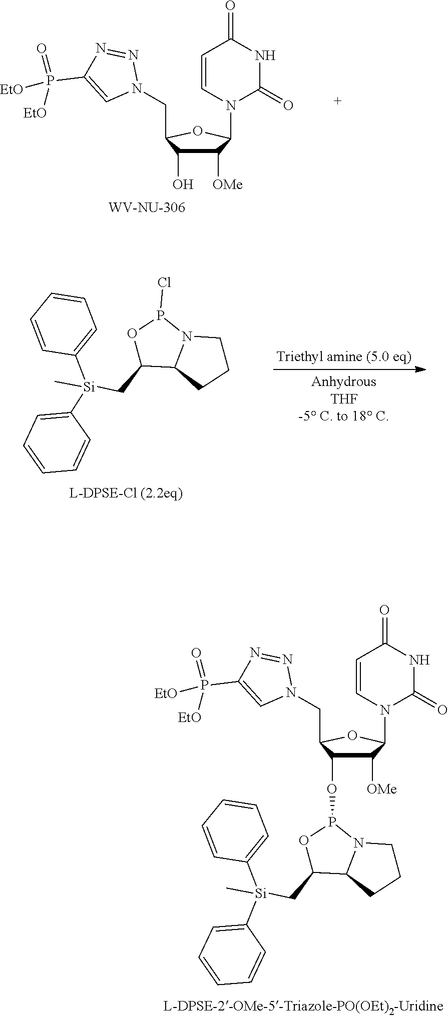

Nucleoside, 2‘—OMe’-5′-triazole-PO(OEt)₂-U (WV-NU-306, 3.12 g) was converted to L-DPSE-2′-OMe-5′-triazole-PO(OEt)₂-Uridine amidite by general procedure (3.18 g, 60% yield) as an off-white solid.

LCMS: C₃₅H₄₆N₆O₉P₂Si (M−H⁻). 783.36

¹H NMR (600 MHz, CDCl₃) δ 8.70 (s, 1H), 8.07 (s, 1H), 7.54 (d, J=7.6 Hz, 2H), 7.50 (d, J=7.7 Hz, 2H), 7.32 (s, 7H), 6.82 (d, J=8.1 Hz, 1H), 5.70 (d, J=8.1 Hz, 1H), 5.37 (d, J=3.1 Hz, 1H), 4.92 (d, J=8.3 Hz, 1H), 4.61 (dd, J=14.6, 2.9 Hz, 1H), 4.44 (s, 2H), 4.20 (s, 5H), 3.79 (d, J=2.4 Hz, 1H), 3.59 (dd, J=7.0, 3.4 Hz, 1H), 3.50 (d, J=2.8 Hz, 1H), 3.38 (s, 3H), 3.21 (dd, J=9.0, 4.4 Hz, 1H), 1.89 (dd, J=8.1, 3.5 Hz, 1H), 1.75 (d, J=9.6 Hz, 1H), 1.65 (dd, J=14.7, 8.5 Hz, 1H), 1.47 (d, J=6.2 Hz, 2H), 1.36 (d, J=5.0 Hz, 6H), 0.67 (s, 3H)

³¹P NMR (243 MHz, CDCl₃) δ 152.69, 6.72

¹³C NMR (151 MHz, CDCl₃) δ 162.85, 149.56, 141.88, 136.59, 135.94, 134.57, 134.36, 132.29, 129.49, 129.43, 127.99, 127.94, 102.90, 93.49, 81.01, 80.59, 79.58, 77.11, 76.90, 76.69, 70.73, 70.67, 67.90, 63.12, 63.08, 58.93, 50.34, 46.74, 46.51, 27.26, 27.21, 25.93, 25.91, 17.75, 16.32, 16.28.

### Synthesis of D-DPSE-2′-OMe-5′-triazole-PO(OEt)₂-2′OMe-Uridine amidite

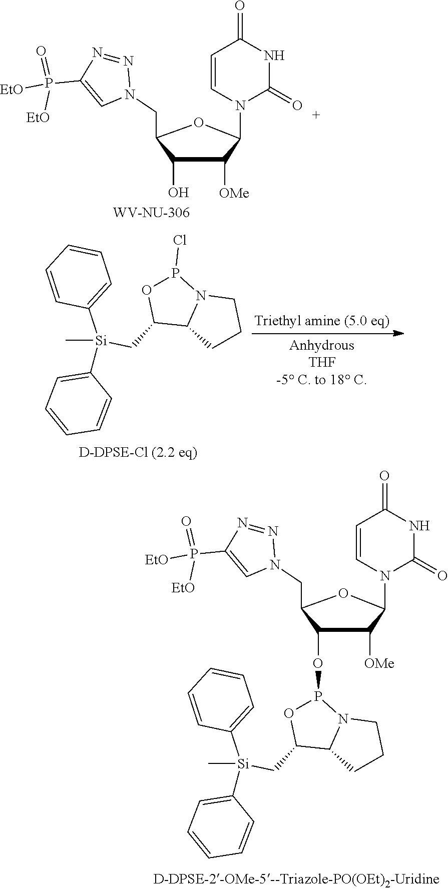

Nucleoside, 2‘—OMe’-5′-triazole-PO(OEt)₂-U (WV-NU-306, 3.11 g) was converted to D-DPSE-2′-OMe-5′-triazole-PO(OEt)₂-2′OMe-Uridine amidite by general procedure (3.35 g, 63% yield) as an off-white solid.

LCMS: C₃₅H₄₆N₆O₉P₂Si (M−H⁻). 783.36

¹H NMR (600 MHz, CDCl₃) δ 9.33 (s, 1H), 8.10 (s, 1H), 7.53 (ddt, J=17.2, 6.5, 1.6 Hz, 4H), 7.33 (dtdd, J=11.0, 8.5, 3.9, 1.9 Hz, 6H), 6.82 (d, J=8.1 Hz, 1H), 5.70 (d, J=8.1 Hz, 1H), 5.47 (d, J=3.3 Hz, 1H), 4.92 (td, J=7.4, 5.4 Hz, 1H), 4.70 (dd, J=14.5, 3.1 Hz, 1H), 4.45 (dd, J=14.6, 6.8 Hz, 1H), 4.37 (ddd, J=9.4, 6.7, 5.6 Hz, 1H), 4.30-4.15 (m, 5H), 3.85 (dd, J=5.6, 3.4 Hz, 1H), 3.59 (ddt, J=14.5, 10.6, 7.5 Hz, 1H), 3.50-3.44 (m, 1H), 3.26 (s, 3H), 3.17 (tdd, J=10.8, 8.7, 4.6 Hz, 1H), 1.85 (dtq, J=12.4, 8.1, 3.6 Hz, 1H), 1.71 (dtd, J=15.0, 8.3, 4.5 Hz, 1H), 1.56 (ddd, J=88.8, 14.6, 7.5 Hz, 2H), 1.45-1.37 (m, 1H), 1.35 (td, J=7.0, 5.7 Hz, 6H), 1.29 (dt, J=9.8, 8.2 Hz, 1H), 0.67 (s, 3H).

³¹P NMR (243 MHz, CDCl₃) δ 154.12, 6.58

¹³C NMR (151 MHz, CDCl₃) δ 162.90, 149.72, 141.36, 136.44, 136.05, 134.61, 134.45, 132.33, 132.11, 129.53, 129.48, 127.99, 127.96, 102.96, 92.53, 81.07, 81.06, 80.62, 80.59, 79.60, 79.53, 70.72, 70.65, 67.50, 67.48, 63.16, 63.12, 58.58, 50.50, 46.93, 46.69, 27.15, 26.02, 26.00, 17.92, 17.89, 16.33, 16.31, 16.29, 16.27.

### Synthesis of L-DPSE-2′-OMe-5′-Vinyl-PO(OEt)₂-Uridine amidite

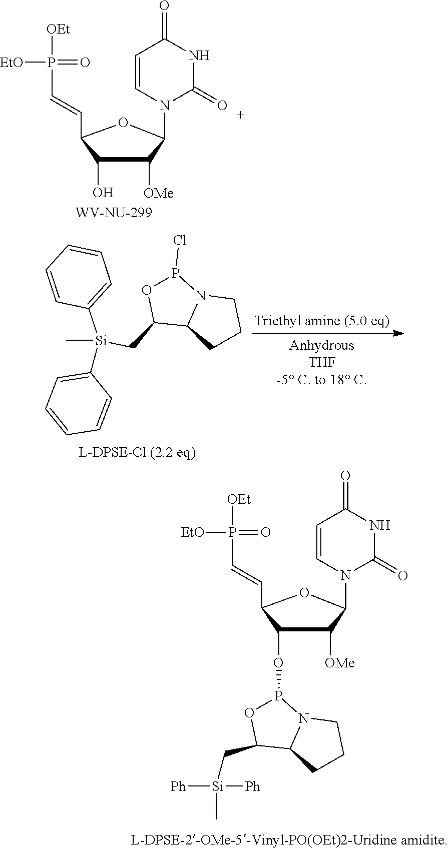

Nucleoside, 2′-OMe-5′-Vinyl-PO(OEt)₂-Uridine (WV-NU-299, 2.45 g) was converted to L-DPSE-2′-OMe-5′-Vinyl-PO(OEt)₂-Uridine amidite. by general procedure (2.58 g, 56% yield) as an off-white solid.

LCMS: C₃₄H₄₅N₃O₉P₂Si (M−H⁻): 728.48

¹H NMR (600 MHz, CDCl₃) δ 8.47 (s, 1H), 7.55-7.50 (m, 4H), 7.40-7.31 (m, 6H), 7.18 (d, J=8.1 Hz, 1H), 6.79 (ddd, J=22.0, 17.2, 4.7 Hz, 1H), 5.99 (ddd, J=19.1, 17.2, 1.7 Hz, 1H), 5.76-5.73 (m, 2H), 4.86 (dt, J=8.1, 6.2 Hz, 1H), 4.43 (dddd, J=6.4, 4.7, 3.1, 1.8 Hz, 1H), 4.26 (ddd, J=8.8, 6.4, 5.2 Hz, 1H), 4.14-4.06 (m, 4H), 3.73 (dd, J=5.2, 3.7 Hz, 1H), 3.54 (dddd, J=14.6, 10.6, 8.1, 6.9 Hz, 1H), 3.41 (s, 4H), 3.18 (tdd, J=10.8, 8.8, 4.5 Hz, 1H), 1.85 (dtq, J=12.4, 8.1, 4.1, 3.5 Hz, 1H), 1.73-1.61 (m, 2H), 1.46 (dd, J=14.6, 6.6 Hz, 1H), 1.43-1.36 (m, 1H), 1.33 (td, J=7.1, 4.7 Hz, 6H), 1.28-1.22 (m, 1H), 0.66 (s, 3H).

³¹P NMR (243 MHz, CDCl₃) δ 152.97, 16.88

¹³C NMR (151 MHz, CDCl₃) δ 162.70, 149.59, 146.68, 146.65, 140.33, 136.48, 135.99, 134.54, 134.39, 129.56, 129.49, 128.01, 127.97, 120.08, 118.84, 102.79, 90.28, 81.72, 81.70, 79.58, 79.51, 73.20, 73.14, 67.73, 67.71, 62.06, 62.02, 61.99, 58.72, 58.71, 46.66, 46.42, 27.17, 25.93, 25.91, 17.80, 17.77, 16.46, 16.41.

### Synthesis of D-DPSE-2′-OMe-5′-Vinyl-PO(OEt)₂-Uridine amidite

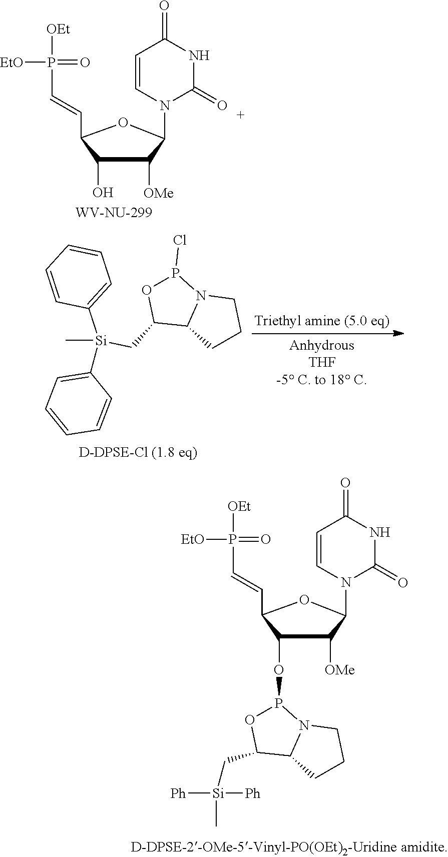

Nucleoside, 2′-OMe-5′-Vinyl-PO(OEt)₂-Uridine (WV-NU-299, 5.39 g) was converted D-DPSE-2′-OMe-5′-Vinyl-PO(OEt)₂-Uridine amidite by general procedure (8.60 g, 85% yield) as an off-white solid.

LCMS: C₃₄H₄₅N₃O₉P₂Si (M−H⁻). 728.29

¹H NMR (600 MHz, CDCl₃) δ 8.98 (s, 1H), 7.52 (ddt, J=8.2, 6.4, 1.6 Hz, 4H), 7.35 (dddd, J=13.7, 8.3, 7.0, 3.9 Hz, 6H), 7.24 (d, J=8.2 Hz, 1H), 6.82 (ddd, J=22.1, 17.2, 4.9 Hz, 1H), 6.01 (ddd, J=18.9, 17.2, 1.7 Hz, 1H), 5.82 (d, J=2.5 Hz, 1H), 5.75 (d, J=8.2 Hz, 1H), 4.89 (td, J=7.5, 5.3 Hz, 1H), 4.59-4.53 (m, 1H), 4.16-4.04 (m, 5H), 3.67 (dd, J=5.1, 2.5 Hz, 1H), 3.53 (ddt, J=14.8, 10.6, 7.6 Hz, 1H), 3.40 (ddt, J=9.5, 7.2, 5.4 Hz, 1H), 3.26 (s, 3H), 3.17 (tdd, J=10.8, 8.8, 4.3 Hz, 1H), 1.87-1.78 (m, 1H), 1.67 (dd, J=14.6, 7.2 Hz, 2H), 1.49 (dd, J=14.5, 7.8 Hz, 1H), 1.33 (q, J=7.0 Hz, 7H), 1.27-1.18 (m, 2H), 0.65 (s, 3H).

³¹P NMR (243 MHz, CDCl₃) δ 152.43, 16.64

¹³C NMR (151 MHz, CDCl₃) δ 161.50, 148.55, 145.48, 145.44, 138.46, 135.24, 134.98, 133.51, 133.41, 128.59, 128.52, 127.00, 126.96, 119.66, 118.41, 101.69, 88.59, 81.37, 77.98, 77.92, 72.29, 72.20, 66.40, 66.38, 61.05, 61.01, 57.44, 45.86, 45.63, 25.95, 24.86, 24.84, 16.92, 16.89, 15.41, 15.37.

### Example 43: Synthesis of D-PSM and L-PSM Amidite

### General Procedure for Synthesis of D-PSM and L-PSM Amidite

Nucleosides (1.0 eq.) in an appropriate size three necked flask was azeotrope with anhydrous toluene (15 mL/g) and anhydrous acetonitrile (15 mL/g) and was dried for 24 hours on high vacuum. To the flask was added anhydrous THF (0.2 M solution) under argon and solution was cooled to 0° C. To the reaction mixture was added triethyl amine (2.2 eq.) followed by addition of D-PSM-Cl or L-PSM-Cl solution (1.8-2 eq, 0.9M) over the period of 5-10 min. The reaction mixture was warmed to room temperature and reaction progress was monitored by HPLC. After disappearance of starting material, the reaction mixture was filtered through fritted glass tube. Reaction flask and precipitate was washed with anhydrous THF. Obtained filtrate was collected and solvent was removed under reduced pressure. The residue was purified by column chromatography (SiO2, 0-100% Ethyl acetate in Hexanes with 1.25% triethyl amine) to give the corresponding D-PSM, L-PSM, or Nu-D-PSM amidites as off-white solid.

### Synthesis of L-PSM-2′-O—C16 lipid-5′-ODMTr-Uridine amidite

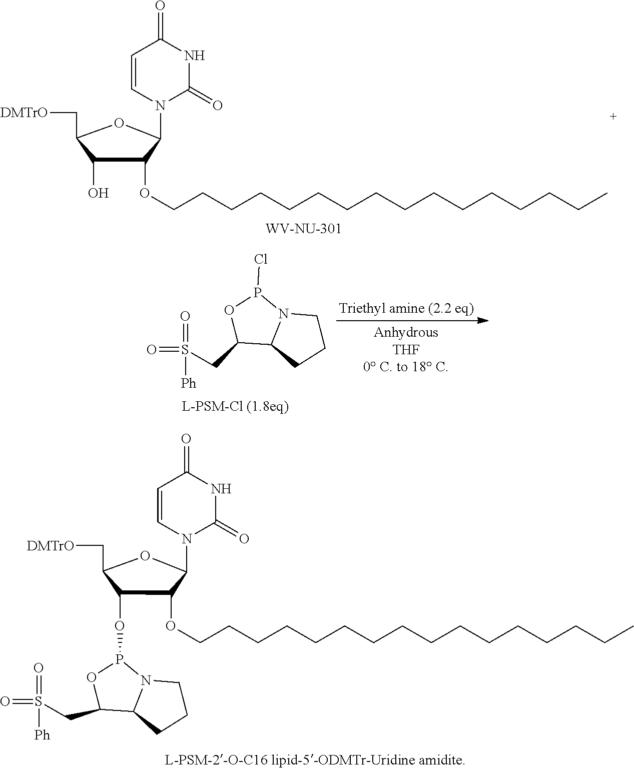

Nucleoside, 2′-O—C16 lipid-5′-ODMTr-Uridine (WV-NU-301, 4.11 g) was converted to L-PSM-2′-O—C16 lipid-5′-ODMTr-Uridine amidite by general procedure (1.21 g, 23% yield) as an off-white solid.

LCMS: C₅₈H₇₆N₃O₁₁P₂S (M−H⁻): 1052.34

¹H NMR (600 MHz, CDCl₃) δ 8.98 (s, 1H), 7.52 (ddt, J=8.2, 6.4, 1.6 Hz, 4H), 7.35 (dddd, J=13.7, 8.3, 7.0, 3.9 Hz, 6H), 7.24 (d, J=8.2 Hz, 1H), 6.82 (ddd, J=22.1, 17.2, 4.9 Hz, 1H), 6.01 (ddd, J=18.9, 17.2, 1.7 Hz, 1H), 5.82 (d, J=2.5 Hz, 1H), 5.75 (d, J=8.2 Hz, 1H), 4.89 (td, J=7.5, 5.3 Hz, 1H), 4.59-4.53 (m, 1H), 4.16-4.04 (m, 5H), 3.67 (dd, J=5.1, 2.5 Hz, 1H), 3.53 (ddt, J=14.8, 10.6, 7.6 Hz, 1H), 3.40 (ddt, J=9.5, 7.2, 5.4 Hz, 1H), 3.26 (s, 3H), 3.17 (tdd, J=10.8, 8.8, 4.3 Hz, 1H), 1.87-1.78 (m, 1H), 1.67 (dd, J=14.6, 7.2 Hz, 2H), 1.49 (dd, J=14.5, 7.8 Hz, 1H), 1.33 (q, J=7.0 Hz, 7H), 1.27-1.18 (m, 2H), 0.65 (s, 3H).

³¹P NMR (243 MHz, CDCl₃) δ 155.06.

¹³C NMR (151 MHz, CDCl₃) δ 162.78, 158.79, 158.75, 149.89, 144.34, 140.27, 139.47, 135.17, 135.04, 133.99, 130.28, 130.20, 129.31, 128.23, 128.12, 128.04, 127.23, 113.33, 102.05, 87.58, 87.18, 82.71, 82.68, 81.49, 74.48, 74.41, 71.14, 70.35, 70.27, 66.20, 66.18, 61.56, 58.12, 58.09, 55.28, 46.56, 46.33, 31.94, 29.72, 29.70, 29.68, 29.66, 29.53, 29.38, 27.34, 26.05, 26.03, 26.01, 22.71, 14.14.

### Synthesis of D-PSM-2′-O—C16 lipid-5′-ODMTr-Uridine amidite

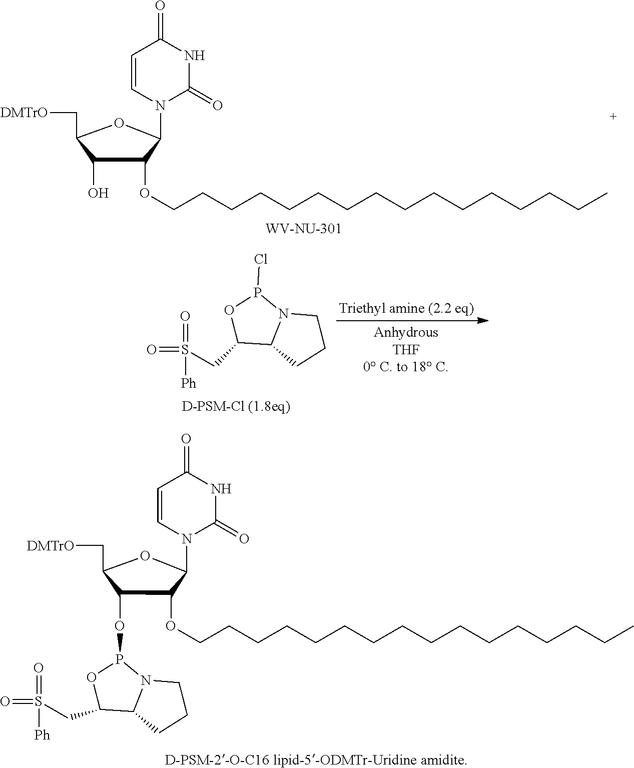

Nucleoside, 2′-O—C16 lipid-5′-ODMTr-Uridine (WV-NU-301, 5.17 g) was converted to D-PSM-2′-O—C16 lipid-5′-ODMTr-Uridine amidite by general procedure (4.80 g, 68% yield) as an off-white solid.

LCMS: C₅₈H₇₆N₃O₁₁P₂S (M−H⁻). 1052.52

¹H NMR (600 MHz, CDCl₃) δ 8.23 (s, 1H), 8.09 (d, J=8.2 Hz, 1H), 7.93-7.89 (m, 2H), 7.68-7.63 (m, 1H), 7.55 (t, J=7.9 Hz, 2H), 7.39-7.36 (m, 2H), 7.33-7.23 (m, 11H), 6.86-6.82 (m, 4H), 5.91 (d, J=1.9 Hz, 1H), 5.22 (d, J=8.2 Hz, 1H), 5.11 (q, J=6.2 Hz, 1H), 4.66 (dd J=9.4, 7.7, 4.7 Hz, 1H), 4.22 (dt, J=7.8, 2.3 Hz, 1H), 3.90 (dd, J=4.8, 1.9 Hz, 1H), 3.80 (d, J=2.2 Hz, 6H), 3.76-3.71 (m, 1H), 3.67 (dd, J=15.7, 9.3, 6.2 Hz, 2H), 3.60 (dd, J=11.3, 2.2 Hz, 1H), 3.52-3.32 (m, 4H), 3.17-3.10 (m, 1H), 1.93-1.74 (m, 2H), 1.70-1.63 (m, 1H), 1.63-1.56 (m, 2H), 1.39-1.31 (m, 2H), 1.31-1.21 (m, 26H), 1.13 (dd, J=11.5, 10.1, 8.3 Hz, 1H), 0.88 (t, J=7.0 Hz, 3H).

³¹P NMR (243 MHz, CDCl₃) δ 156.17.

13C NMR (151 MHz, CDCl₃) δ 162.86, 158.77, 158.73, 149.77, 144.21, 140.23, 139.66, 135.11, 134.95, 134.06, 130.30, 130.27, 129.38, 128.26, 128.08, 128.02, 127.21, 113.30, 113.27, 101.86, 88.00, 87.09, 81.85, 81.59, 73.98, 73.92, 70.93, 69.18, 66.13, 60.30, 58.12, 58.09, 55.27, 46.62, 46.39, 31.94, 29.81, 29.73, 29.70, 29.68, 29.57, 29.38, 27.37, 26.11, 26.04, 26.02, 22.71, 14.14.

### Synthesis of 5′-(R)—C-Me-5′-ODMTr-2′OMe-A(Bz)-D-PSM

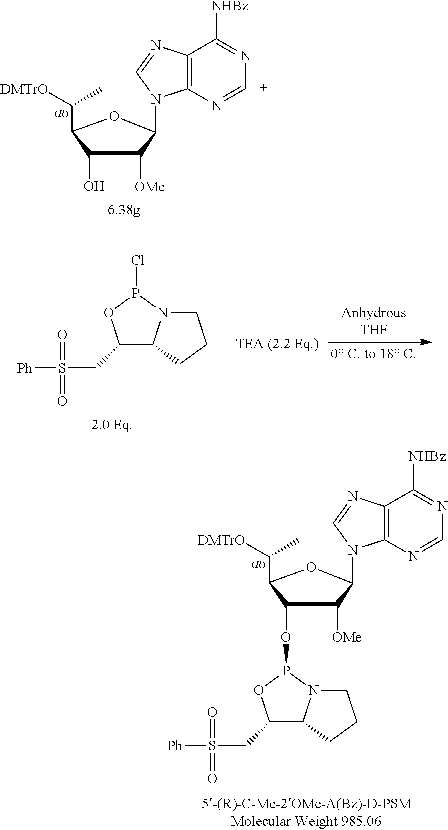

Nucleoside 5′-(R)—C-Me-5′-ODMTr-2′OMe-A(Bz) (6.38 g) was converted to 5′-(R)—C-Me-5′-ODMTr-2′OMe-A(Bz)-D-PSM-amidite by general procedure (6.1 g, 72.9% yield) as an off-white solid.

LCMS: C₅₂H₅₃N₆O₁₀P₂S (M−H⁻). 983.81

¹H NMR (600 MHz, CDCl₃) δ 8.96 (s, 1H), 8.66 (s, 1H), 8.00 (d, J=7.5 Hz, 2H), 7.98-7.94 (m, 2H), 7.90 (s, 1H), 7.60 (d, J=11.4 Hz, 2H), 7.52 (s, 6H), 7.44-7.38 (m, 4H), 7.26 (s, 3H), 7.21 (s, 1H), 6.83 (s, 4H), 5.96 (d, J=7.1 Hz, 1H), 5.23 (d, J=6.6 Hz, 1H), 4.76 (d, J=16.3 Hz, 1H), 4.34 (d, J=11.7 Hz, 1H), 3.77 (s, 7H), 3.75-3.57 (m, 3H), 3.52 (s, 1H), 3.43 (d, J=14.5 Hz, 1H), 3.31 (d, J=8.8 Hz, 1H), 3.18 (s, 3H), 1.89 (d, J=52.7 Hz, 3H), 1.67 (s, 1H), 1.17 (s, 1H), 0.92 (s, 3H).

³¹P NMR (243 MHz, CDCl₃) δ 156.98.

¹³C NMR (151 MHz, CDCl₃) δ 164.64, 158.70, 152.53, 151.80, 149.55, 143.05, 139.61, 136.86, 136.84, 134.11, 133.78, 132.90, 130.59, 129.43, 129.00, 128.58, 128.37, 127.91, 127.82, 126.99, 113.17, 113.14, 88.44, 86.72, 86.61, 80.33, 73.72, 73.66, 71.46, 71.36, 69.59, 66.22, 66.20, 60.50, 58.50, 58.28, 58.26, 55.35, 47.04, 46.80, 27.46, 26.27, 21.23-21.10 (m), 18.41, 14.31.

### Synthesis of 5′-(S)—C-Me-5′-ODMTr-2′OMe-A(Bz)-D-PSM

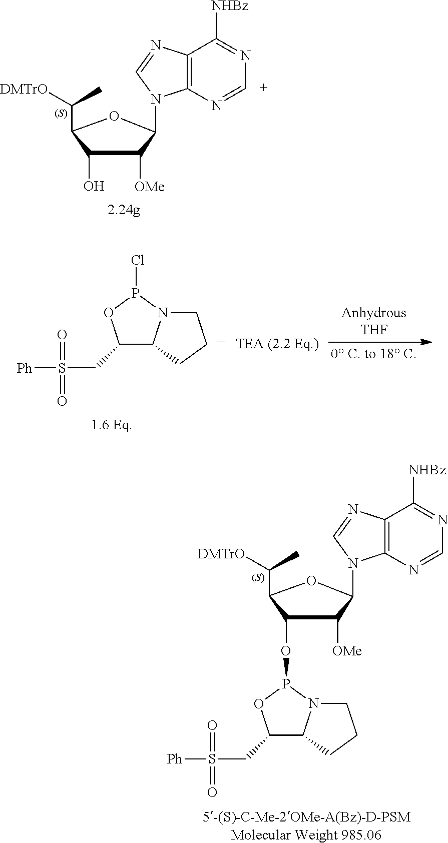

Nucleoside 5′-(S)—C-Me-5′-ODMTr-2′OMe-A(Bz) (2.24 g) was converted to 5′-(S)—C-Me-5′-ODMTr-2′OMe-A(Bz)-D-PSM-amidite by general procedure (2.47 g, 78.6% yield) as an off-white solid.

LCMS: C₅₂H₅₃N₆O₁₀P₂S (M−H⁻). 983.24

¹H NMR (600 MHz, CDCl₃) δ 9.02-8.99 (m, 1H), 8.79 (s, 1H), 8.28 (s, 1H), 8.05-8.01 (m, 2H), 7.97-7.91 (m, 2H), 7.64-7.57 (m, 2H), 7.56-7.49 (m, 6H), 7.40 (t, J=8.6 Hz, 4H), 7.24 (dd, J=8.4, 6.9 Hz, 2H), 7.22-7.15 (m, 1H), 6.83-6.75 (m, 4H), 6.06 (d, J=6.3 Hz, 1H), 5.12 (q, J=6.2 Hz, 1H), 4.77-4.68 (m, 2H), 4.11 (q, J=7.1 Hz, 1H), 4.03 (t, J=3.4 Hz, 1H), 3.78 (d, J=5.0 Hz, 5H), 3.78-3.69 (m, 1H), 3.72-3.65 (m, 1H), 3.51 (dd, J=14.5, 6.8 Hz, 1H), 3.48-3.36 (m, 2H), 3.33 (s, 3H), 3.14 (tdd, J=10.1, 8.8, 3.9 Hz, 1H), 2.04 (s, 2H), 1.91-1.83 (m, 1H), 1.78 (td, J=11.8, 7.5 Hz, 1H), 1.70-1.62 (m, 1H), 1.25 (t, J=7.1 Hz, 2H), 1.16-1.07 (m, 1H), 0.93 (d, J=6.4 Hz, 3H).

³¹P NMR (243 MHz, CDCl₃) δ 155.82.

¹³C NMR (151 MHz, CDCl₃) δ 158.69, 158.64, 149.68, 146.15, 142.65, 139.62, 136.85, 136.52, 134.16, 133.84, 132.93, 130.75, 130.62, 129.47, 129.04, 128.57, 128.37, 127.98, 127.78, 126.94, 123.97, 113.11, 113.09, 88.02, 87.99, 86.92, 86.08, 81.46, 81.44, 73.98, 73.92, 70.62, 70.53, 69.30, 66.24, 66.22, 60.53, 58.48, 58.33, 58.30, 55.36, 46.92, 46.68, 27.48, 26.21, 26.18, 21.19, 17.67, 14.34.

### Synthesis of 2′O-C16-U-L-PSM

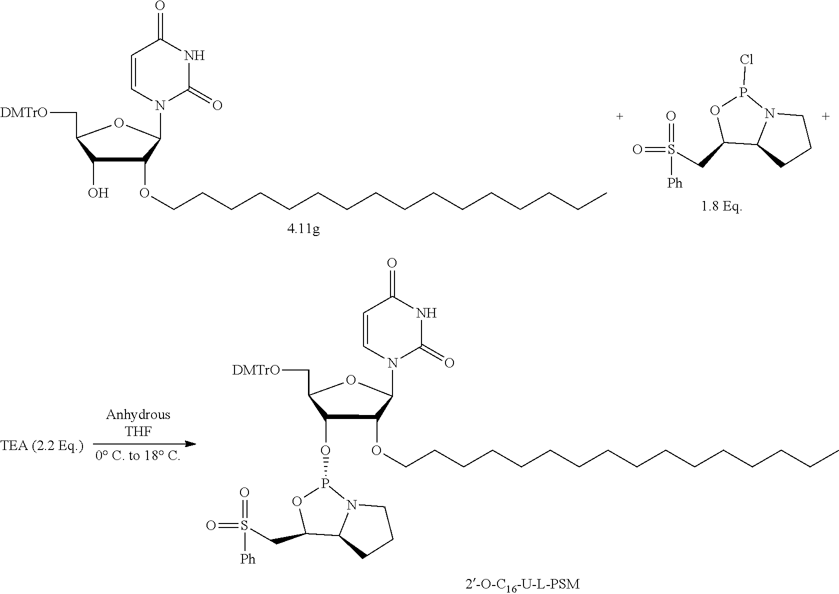

Nucleoside 2′O-C16-U (4.11 g) was converted to 2′O-C16-U-L-PSM-amidite by general procedure (1.21 g, 23% yield) as an off-white solid.

LCMS: C₅₈H₇₆N₃O₁₁P₂S (M−H⁻): 1052.34

¹H NMR (600 MHz, CDCl₃) δ 8.98 (s, 1H), 7.52 (ddt, J=8.2, 6.4, 1.6 Hz, 4H), 7.35 (dddd, J=13.7, 8.3, 7.0, 3.9 Hz, 6H), 7.24 (d, J=8.2 Hz, 1H), 6.82 (ddd, J=22.1, 17.2, 4.9 Hz, 1H), 6.01 (ddd, J=18.9, 17.2, 1.7 Hz, 1H), 5.82 (d, J=2.5 Hz, 1H), 5.75 (d, J=8.2 Hz, 1H), 4.89 (td, J=7.5, 5.3 Hz, 1H), 4.59-4.53 (m, 1H), 4.16-4.04 (m, 5H), 3.67 (dd, J=5.1, 2.5 Hz, 1H), 3.53 (ddt, J=14.8, 10.6, 7.6 Hz, 1H), 3.40 (ddt, J=9.5, 7.2, 5.4 Hz, 1H), 3.26 (s, 3H), 3.17 (tdd, J=10.8, 8.8, 4.3 Hz, 1H), 1.87-1.78 (m, 1H), 1.67 (dd, J=14.6, 7.2 Hz, 2H), 1.49 (dd, J=14.5, 7.8 Hz, 1H), 1.33 (q, J=7.0 Hz, 7H), 1.27-1.18 (m, 2H), 0.65 (s, 3H).

³¹P NMR (243 MHz, CDCl₃) δ 155.06.

¹³C NMR (151 MHz, CDCl₃) δ 162.78, 158.79, 158.75, 149.89, 144.34, 140.27, 139.47, 135.17, 135.04, 133.99, 130.28, 130.20, 129.31, 128.23, 128.12, 128.04, 127.23, 113.33, 102.05, 87.58, 87.18, 82.71, 82.68, 81.49, 74.48, 74.41, 71.14, 70.35, 70.27, 66.20, 66.18, 61.56, 58.12, 58.09, 55.28, 46.56, 46.33, 31.94, 29.72, 29.70, 29.68, 29.66, 29.53, 29.38, 27.34, 26.05, 26.03, 26.01, 22.71, 14.14.

### Synthesis of 2′O-C16-U-D-PSM

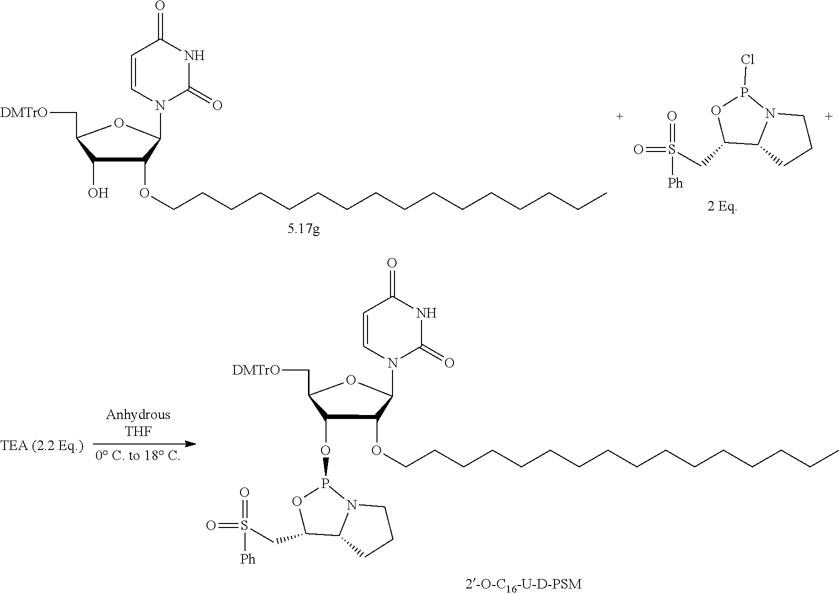

Nucleoside 2′O—C16-U (5.17 g) was converted to 2′O—C16-U-D-PSM-amidite by general procedure (4.80 g, 68% yield) as an off-white solid.

LCMS: C₅₈H₇₆N₃O₁₁P₂S (M−H⁻): 1052.52

¹H NMR (600 MHz, CDCl₃) δ 8.23 (s, 1H), 8.09 (d, J=8.2 Hz, 1H), 7.93-7.89 (m, 2H), 7.68-7.63 (m, 1H), 7.55 (t, J=7.9 Hz, 2H), 7.39-7.36 (m, 2H), 7.33-7.23 (m, 11H), 6.86-6.82 (m, 4H), 5.91 (d, J=1.9 Hz, 1H), 5.22 (d, J=8.2 Hz, 1H), 5.11 (q, J=6.2 Hz, 1H), 4.66 (ddd, J=9.4, 7.7, 4.7 Hz, 1H), 4.22 (dt, J=7.8, 2.3 Hz, 1H), 3.90 (dd, J=4.8, 1.9 Hz, 1H), 3.80 (d, J=2.2 Hz, 6H), 3.76-3.71 (m, 1H), 3.67 (ddt, J=15.7, 9.3, 6.2 Hz, 2H), 3.60 (dd, J=11.3, 2.2 Hz, 1H), 3.52-3.32 (m, 4H), 3.17-3.10 (m, 1H), 1.93-1.74 (m, 2H), 1.70-1.63 (m, 1H), 1.63-1.56 (m, 2H), 1.39-1.31 (m, 2H), 1.31-1.21 (m, 26H), 1.13 (dtd, J=11.5, 10.1, 8.3 Hz, 1H), 0.88 (t, J=7.0 Hz, 3H).

³¹P NMR (243 MHz, CDCl₃) δ 156.17.

¹³C NMR (151 MHz, CDCl₃) δ 162.86, 158.77, 158.73, 149.77, 144.21, 140.23, 139.66, 135.11, 134.95, 134.06, 130.30, 130.27, 129.38, 128.26, 128.08, 128.02, 127.21, 113.30, 113.27, 101.86, 88.00, 87.09, 81.85, 81.59, 73.98, 73.92, 70.93, 69.18, 66.13, 60.30, 58.12, 58.09, 55.27, 46.62, 46.39, 31.94, 29.81, 29.73, 29.70, 29.68, 29.57, 29.38, 27.37, 26.11, 26.04, 26.02, 22.71, 14.14.

### Example 44: Synthesis of Stereorandom CNE Amidite

### General Procedure for Synthesis of Stereorandom CNE Amidite

Nucleosides (1.0 eq.) in an appropriate size three necked flask was azeotroped with anhydrous toluene (15 mL/g) and anhydrous acetonitrile (15 mL/g) and was dried for 24 hours on high vacuum. To the flask was added anhydrous acetonitrile (0.1 M solution) under argon at room temperature. To the reaction mixture was added 5-ethylsulfanyl-1H-tetrazole (1.0 eq.) followed by dropwise addition of 3-bis(diisopropylamino)phosphanyloxypropanenitrile (1.2-1.5 eq.) over the period of 5-10 min. The reaction progress was monitored by HPLC. After disappearance of starting material, the reaction mixture was filtered through fritted glass tube. Reaction flask and precipitate was washed with anhydrous THF. Obtained filtrate was collected and solvent was removed under reduced pressure. The residue was purified by column chromatography (SiO2, 0-100% Ethyl acetate in Hexanes with 5% triethyl amine) to give the corresponding stereorandom CNE or nucleoside CNE amidite as off-white solid.

### Synthesis of 2′-OMe-5′-triazole-PO(OEt)₂-3′-CNE Uridine Amidite

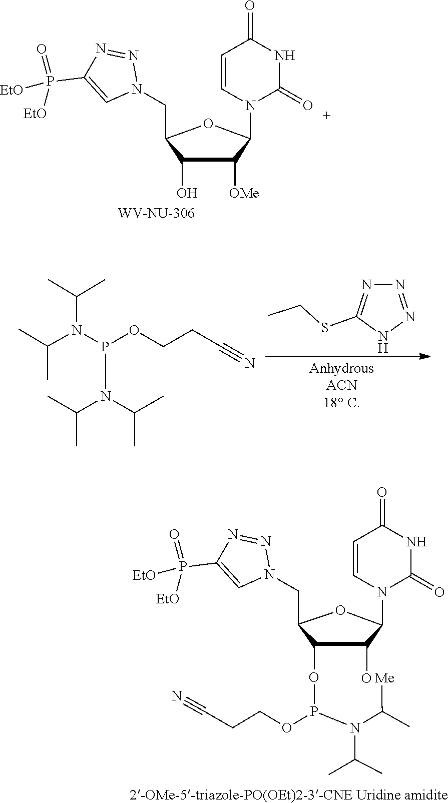

Nucleoside, 2‘—OMe’-5′-triazole-PO(OEt)₂-U (WV-NU-306, 1.81 g) was converted to 2′-OMe-5′-triazole-PO(OEt)₂-3′-CNE Uridine amidite by general procedure (1.82 g, 73% yield) as an off-white solid.

LCMS: C₂₅H₄₁N₇O₉P₂(M−H⁻): 644.50

¹H NMR (600 MHz, CDCl₃) δ 8.92 (s, 1H), 8.19 (s, 1H), 8.16 (s, 1H), 6.91 (d, J=8.1 Hz, 1H), 6.88 (d, J=8.1 Hz, 1H), 5.71 (d, J=8.1 Hz, 1H), 5.68 (d, J=8.1 Hz, 1H), 5.63 (d, J=3.6 Hz, 1H), 5.58 (d, J=3.6 Hz, 1H), 4.94 (dd, J=14.6, 3.1 Hz, 1H), 4.83 (dd, J=14.5, 3.2 Hz, 1H), 4.76 (dd, J=14.6, 6.2 Hz, 1H), 4.69 (dd, J=14.5, 6.8 Hz, 1H), 4.45 (td, J=6.3, 3.1 Hz, 1H), 4.40 (td, J=6.6, 3.2 Hz, 1H), 4.34 (dd, J=14.2, 9.7, 5.8 Hz, 2H), 4.27-4.14 (m, 8H), 4.11 (dd, J=5.4, 3.6 Hz, 1H), 3.97 (dd, J=5.4, 3.7 Hz, 1H), 3.95-3.90 (m, 2H), 3.81 (dd, J=10.4, 8.1, 6.1 Hz, 1H), 3.75-3.69 (m, 1H), 3.69-3.60 (m, 4H), 3.50 (s, 3H), 3.47 (s, 2H), 2.84-2.61 (m, 4H), 1.35 (tq, J=7.8, 3.7 Hz, 12H), 1.27 (dd, J=8.2, 6.8 Hz, 2H), 1.23-1.16 (m, 24H).

³¹P NMR (243 MHz, CDCl₃) δ 150.53, 150.42, 14.17, 6.59, 6.54.

¹³C NMR (151 MHz, CDCl₃) δ 162.66, 149.82, 141.50, 140.92, 138.48, 136.90, 132.51, 132.33, 132.11, 118.15, 117.80, 103.11, 102.97, 92.77, 91.68, 81.21, 81.19, 80.93, 80.89, 80.85, 80.78, 71.63, 71.36, 71.27, 63.15, 63.11, 58.82, 58.75, 58.55, 58.43, 57.65, 57.52, 50.90, 50.59, 43.45, 43.36, 24.72, 24.70, 24.67, 24.65, 24.62, 24.56, 20.46, 20.41, 16.32, 16.28, 16.25.

### Synthesis of 2′-O—C16 lipid-5′-ODMTr-3′-CNE Uridine Amidite

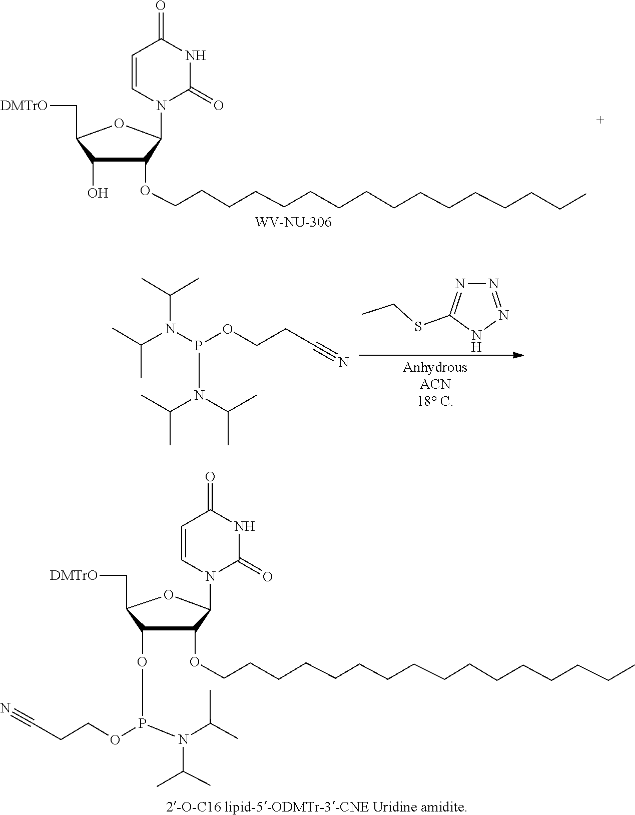

Nucleoside, 2′-O—C16 lipid-5′-ODMTr-Uridine (WV-NU-301, 4.81 g) was converted to 2′-O—C16 lipid-5′-ODMTr-3′-CNE Uridine amidite by general procedure (4.10 g, 68% yield) as an off-white solid.

LCMS: C₅₈H₇₆N₃O₁₁P₂S (M−H⁻). 1052.52

¹H NMR (600 MHz, CDCl₃) δ 8.23 (s, 1H), 8.01 (dd, J=53.9, 8.2 Hz, 1H), 7.39 (dd, J=21.3, 7.7 Hz, 2H), 7.30 (h, J=4.9 Hz, 4H), 7.26 (s, 6H), 6.84 (s, 4H), 5.95 (dd, J=25.5, 2.6 Hz, 1H), 5.21 (t, J=8.2 Hz, 1H), 4.60-4.42 (m, 1H), 4.28-4.18 (m, 1H), 4.00 (ddd, J=12.8, 4.9, 2.5 Hz, 1H), 3.95-3.88 (m, 1H), 3.80 (d, J=3.3 Hz, 6H), 3.78-3.64 (m, 2H), 3.63-3.53 (m, 4H), 3.45 (ddd, J=17.9, 11.1, 2.5 Hz, 1H), 2.63 (q, J=6.4 Hz, 1H), 2.42 (t, J=6.3 Hz, 1H), 1.60 (dhept, J=13.8, 7.1 Hz, 2H), 1.25 (s, 25H), 1.17 (s, 8H), 1.05 (s, 3H), 0.88 (s, 3H).

³¹P NMR (243 MHz, CDCl₃) δ 150.12.

¹³C NMR (151 MHz, CDCl₃) δ 162.95, 162.88, 158.77, 158.74, 140.22, 140.14, 135.26, 135.07, 130.32, 130.30, 130.27, 128.33, 128.29, 127.99, 127.98, 127.23, 113.26, 113.25, 113.23, 102.02, 101.90, 88.17, 87.98, 87.14, 86.96, 82.39, 82.36, 82.16, 82.10, 81.36, 71.19, 70.95, 69.84, 69.74, 61.44, 60.82, 58.56, 58.44, 57.99, 57.86, 55.28, 55.26, 43.34, 43.26, 43.24, 43.16, 31.94, 29.86, 29.84, 29.72, 29.68, 29.66, 29.62, 29.58, 29.54, 29.38, 26.07, 26.06, 24.71, 24.67, 24.62, 24.59, 24.54, 22.71, 20.50, 20.46, 20.32, 20.28, 14.14.

### Synthesis of 5′-(R)—C-Me-5′-ODMTr-2′OMe-A(Bz)-CNE

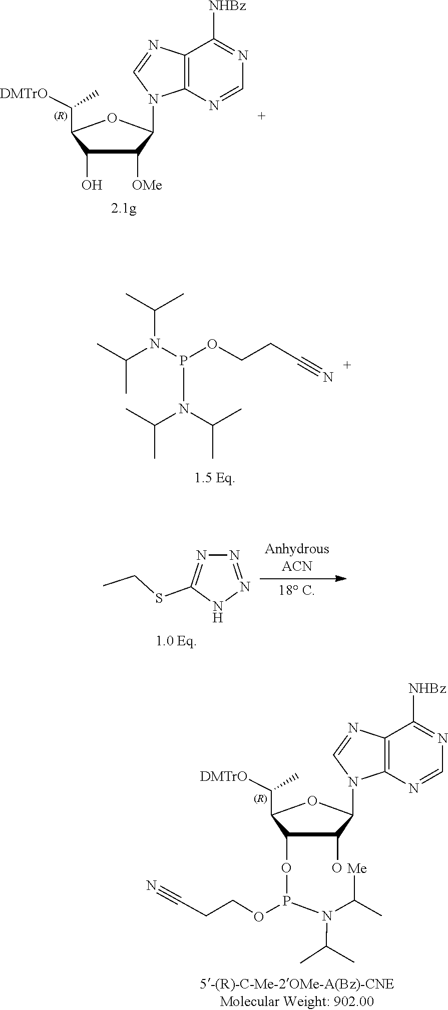

Nucleoside 5′-(R)—C-Me-5′-ODMTr-2′OMe-A(Bz) (2.1 g) was converted to 5′-(R)—C-Me-5′-ODMTr-2′OMe-A(Bz)-CNE-amidite by general procedure (2.16 g, 85.6% yield) as an off-white solid.

LCMS: C₄₉H₅₆N₇O₈P (M−H⁻). 900.79

¹H NMR (600 MHz, CDCl₃) δ 8.90 (s, 1H), 8.69 (d, J=1.5 Hz, 1H), 8.03-7.97 (m, 2H), 7.86 (d, J=1.6 Hz, 1H), 7.63-7.58 (m, 1H), 7.52 (ddd, J=7.5, 4.2, 2.9 Hz, 4H), 7.44-7.38 (m, 4H), 7.30-7.26 (m, 2H), 7.23-7.18 (m, 1H), 6.85-6.79 (m, 4H), 5.99 (t, J=6.6 Hz, 1H), 4.72 (dddd, J=42.4, 10.6, 4.6, 2.6 Hz, 1H), 4.38 (td, J=6.9, 4.6 Hz, 1H), 4.25-4.15 (m, 1H), 3.92-3.79 (m, 2H), 3.78 (t, J=2.2 Hz, 6H), 3.71 (dtdd, J=27.3, 13.2, 6.3, 3.5 Hz, 3H), 3.31 (d, J=17.0 Hz, 3H), 2.66-2.48 (m, 2H), 1.26-1.21 (m, 13H), 0.89 (dd, J=7.9, 6.2 Hz, 3H).

³¹P NMR (243 MHz, CDCl₃) δ 150.45, 149.14.

¹³C NMR (151 MHz, CDCl₃) δ 164.60, 158.73, 158.70, 152.70, 152.67, 151.89, 149.56, 146.27, 142.63, 133.83, 132.93, 130.70, 130.67, 130.61, 130.59, 129.04, 128.62, 128.56, 127.92, 127.84, 127.01, 126.99, 123.79, 113.19, 113.15, 88.51, 88.45, 88.42, 86.96, 86.86, 86.79, 86.68, 81.02, 80.91, 71.23, 71.12, 69.73, 69.65, 60.53, 58.57, 58.49, 58.45, 58.07, 55.38, 55.37, 43.56, 43.48, 24.86, 24.80, 24.75, 21.19, 20.45, 20.41, 18.20, 17.98, 14.34.

### Synthesis of 5′-(R)—C-Me-5′-ODMTr-2′Fd-A(Bz)-CNE

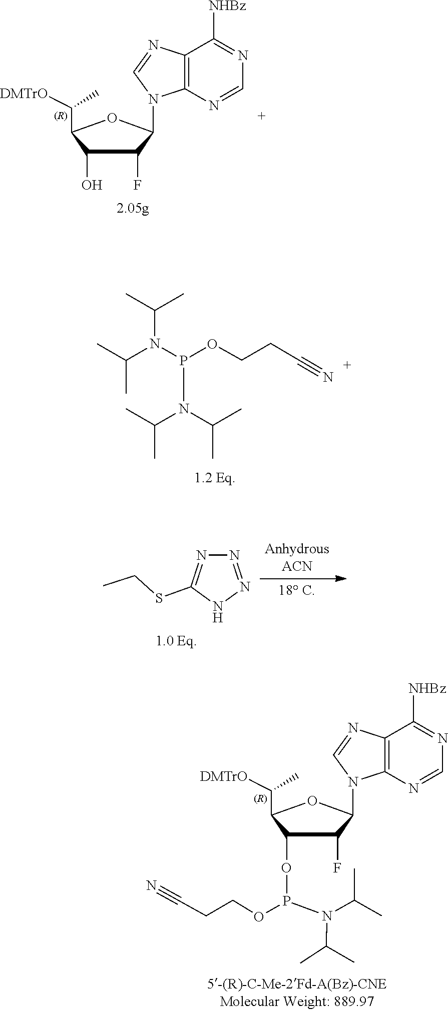

Nucleoside 5′-(R)—C-Me-5′-ODMTr-2′Fd-A(Bz) (2.05 g) was converted to 5′-(R)—C-Me-5′-ODMTr-2′Fd-A(Bz)-CNE-amidite by general procedure (2.39 g, 90.4% yield) as an off-white solid.

LCMS: C₄₈H₅₃FN₇O₇P (M−H⁻). 888.66

¹H NMR (600 MHz, CDCl₃) δ 8.97 (s, 1H), 8.68 (d, J=11.1 Hz, 1H), 8.05-7.98 (m, 2H), 7.63-7.58 (m, 1H), 7.55-7.43 (m, 3H), 7.40-7.31 (m, 3H), 7.27-7.15 (m, 2H), 6.83-6.74 (m, 3H), 6.20-6.13 (m, 1H), 4.16-4.07 (m, 1H), 3.95-3.79 (m, 1H), 3.77 (t, J=2.9 Hz, 4H), 3.75-3.57 (m, 2H), 2.62 (td, J=6.3, 1.5 Hz, 1H), 2.60-2.48 (m, 1H), 2.04 (s, 1H), 1.32-1.17 (m, 8H), 1.16 (d, J=6.8 Hz, 2H), 0.80 (d, J=6.4 Hz, 1H), 0.77 (d, J=6.3 Hz, 1H).

³¹P NMR (243 MHz, CDCl₃) δ 150.82 (d, J=10.6 Hz), 149.71 (d, J=12.6 Hz).

¹³C NMR (151 MHz, CDCl₃) δ 158.69, 158.67, 151.42, 151.34, 149.85, 146.07, 142.58, 136.79, 136.75, 136.73, 133.75, 133.72, 132.99, 132.97, 130.63, 130.62, 130.58, 130.55, 129.04, 128.55, 128.49, 127.98, 127.83, 127.79, 126.97, 126.91, 123.68, 117.65, 113.17, 113.15, 113.13, 87.70, 87.46, 87.24, 86.85, 86.81, 86.35, 86.32, 85.60, 85.56, 70.64, 70.58, 68.96, 68.82, 60.52, 58.85, 58.82, 58.73, 58.70, 55.37, 55.35, 43.58, 43.56, 43.50, 43.48, 24.89, 24.84, 24.81, 24.76, 24.71, 24.66, 24.62, 20.54, 20.50, 20.49, 20.44, 16.40, 16.38, 14.34.

### Synthesis of 5′-(R)—C-Me-5′-ODMTr-2′Fd-U-CNE

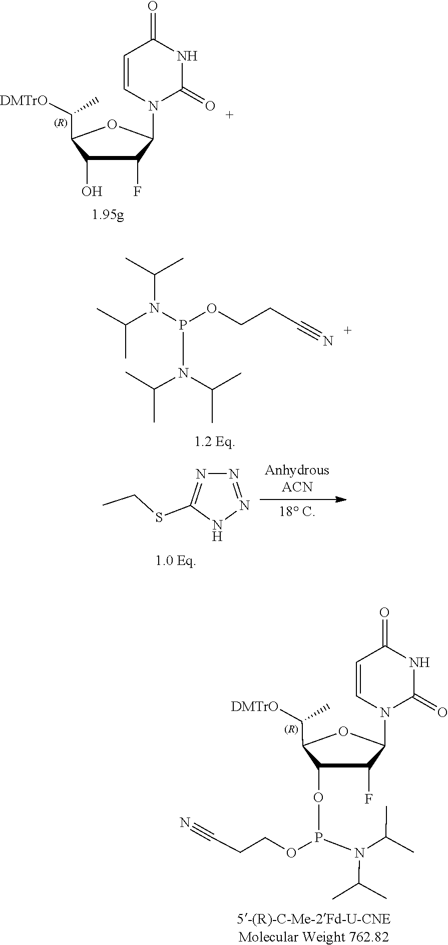

Nucleoside 5′-(R)—C-Me-5′-ODMTr-2′Fd-U (1.95 g) was converted to 5′-(R)—C-Me-5′-ODMTr-2′Fd-U-CNE-amidite by general procedure (782 mg, 29.6% yield) as an off-white solid.

LCMS: C₄₀H₄₈FN₄O₈P (M−H⁻). 761.46

¹H NMR (600 MHz, CDCl₃) δ 8.33 (d, J=5.3 Hz, 1H), 7.48 (dt, J=8.2, 1.3 Hz, 2H), 7.43-7.33 (m, 4H), 7.31-7.23 (m, 2H), 7.23-7.17 (m, 1H), 6.86-6.79 (m, 4H), 5.93 (ddd, J=24.7, 17.0, 2.7 Hz, 1H), 5.14 (dq, J=8.3, 2.5 Hz, 1H), 5.12-5.03 (m, 1H), 4.89-4.75 (m, 1H), 4.12 (q, J=7.1 Hz, 1H), 4.01-3.89 (m, 2H), 3.91-3.71 (m, 5H), 3.79 (s, 2H), 3.71-3.62 (m, 3H), 2.67 (td, J=5.9, 1.5 Hz, 1H), 2.61 (t, J=6.2 Hz, 1H), 2.04 (s, 2H), 1.29-1.18 (m, 13H), 0.96 (dd, J=6.7, 3.4 Hz, 3H).

³¹P NMR (243 MHz, CDCl₃) δ 150.55 (d, J=14.5 Hz), 150.28 (d, J=11.4 Hz).

¹³C NMR (151 MHz, CDCl₃) δ 162.87, 158.83, 158.73, 149.98, 149.95, 146.41, 146.34, 140.70, 140.66, 136.30, 136.28, 136.20, 130.71, 130.67, 130.51, 128.27, 128.04, 127.08, 127.03, 117.90, 117.67, 113.39, 113.29, 102.66, 102.55, 93.52, 92.25, 88.31, 88.08, 87.30, 86.10, 86.07, 85.13, 85.08, 70.04, 69.19, 68.93, 60.57, 58.45, 58.36, 58.32, 58.23, 55.44, 55.42, 55.39, 43.68, 43.60, 43.57, 43.49, 24.92, 24.89, 24.84, 24.80, 24.75, 24.67, 24.62, 21.22, 20.69, 20.65, 20.63, 17.78, 17.67, 14.37.

### Synthesis of 5′-ODMTr-(S)-GNA-G(iBu)-CNE

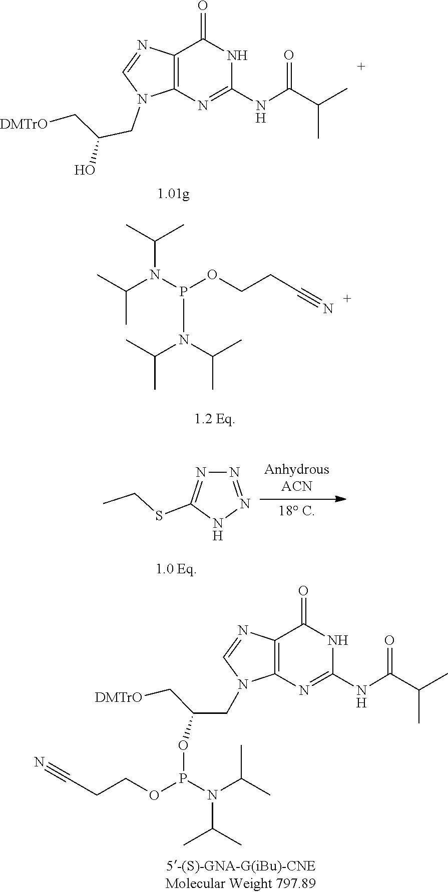

Nucleoside 5′-ODMTr-(S)-GNA-G(iBu) (1.01 g) was converted to 5′-ODMTr-(S)-GNA-G(iBu)-CNE-amidite by general procedure (707 mg, 52.4% yield) as an off-white solid.

LCMS: C₄₂H₅₂N₇O₇P (M−H⁻): 796.63

¹H NMR (600 MHz, CDCl₃) δ 11.85 (s, 1H), 7.44 (ddt, J=9.6, 6.3, 1.3 Hz, 2H), 7.32-7.28 (m, 4H), 7.28 (q, J=1.1 Hz, 1H), 7.27-7.24 (m, 2H), 7.23-7.19 (m, 1H), 6.80 (ddd, J=8.9, 3.8, 2.9 Hz, 4H), 4.37-4.24 (m, 2H), 3.78 (dd, J=2.6, 1.7 Hz, 6H), 3.64-3.52 (m, 3H), 3.16-3.05 (m, 2H), 2.69-2.57 (m, 1H), 2.52 (pd, J=6.9, 5.8 Hz, 1H), 2.49-2.44 (m, 1H), 2.00 (s, 3H), 1.23 (s, 1H), 1.23-1.19 (m, 5H), 1.16-1.12 (m, 9H), 1.11 (d, J=6.8 Hz, 3H).

³¹P NMR (243 MHz, CDCl₃) δ 148.57, 148.47.

¹³C NMR (151 MHz, CDCl₃) δ 178.37, 158.71, 155.69, 148.59, 147.03, 144.90, 140.04, 139.94, 135.98, 135.79, 130.18, 130.13, 130.09, 130.08, 128.19, 128.11, 128.00, 127.94, 127.07, 127.04, 121.04, 117.80, 113.31, 113.29, 113.23, 113.19, 86.44, 86.21, 71.17, 71.09, 63.71, 63.56, 57.50, 57.43, 57.37, 57.31, 55.40, 46.51, 45.77, 43.32, 43.27, 43.24, 43.19, 36.50, 36.34, 24.81, 24.77, 24.72, 24.67, 20.58, 20.53, 20.48, 19.07, 19.05, 2.03.

### Synthesis of 5′-ODMTr-(S)-GNA-T-CNE

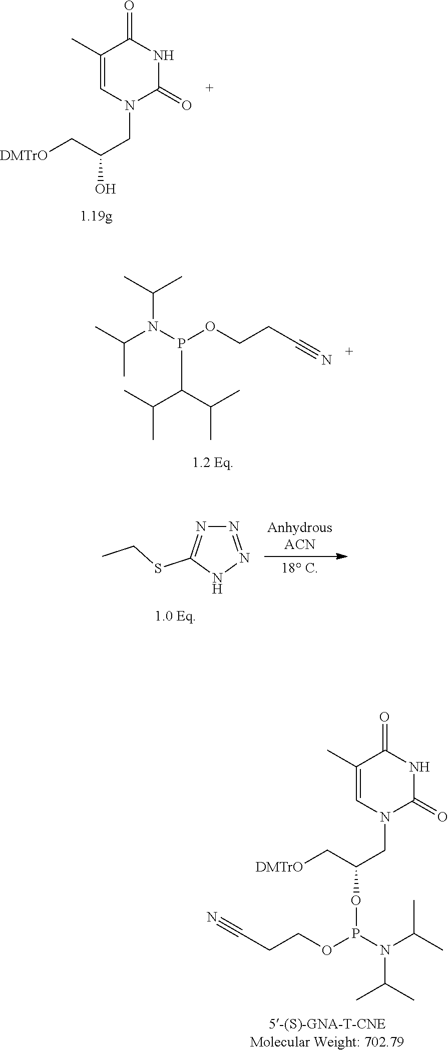

Nucleoside 5′-ODMTr-(S)-GNA-T (1.19 g) was converted to 5′-ODMTr-(S)-GNA-T-CNE-amidite by general procedure (1.27 g, 76.3% yield) as an off-white solid.

LCMS: C₃₈H₄₇N₄O₇P (M−H⁻): 701.66

¹H NMR (600 MHz, CDCl₃) δ 7.45 (ddd, J=8.2, 4.1, 1.3 Hz, 2H), 7.32 (tt, J=8.1, 3.3 Hz, 4H), 7.30-7.27 (m, 2H), 7.24-7.18 (m, 1H), 7.04 (dd, J=5.6, 1.4 Hz, 1H), 6.83 (ddd, J=11.0, 7.8, 1.7 Hz, 4H), 4.22 (tt, J=14.2, 4.5 Hz, 2H), 4.06 (dd, J=13.9, 4.7 Hz, 1H), 3.79 (d, J=5.4 Hz, 7H), 3.75-3.51 (m, 5H), 3.33-3.21 (m, 1H), 3.17 (ddd, J=31.9, 10.0, 4.8 Hz, 1H), 2.59 (q, J=6.2 Hz, 1H), 2.40 (t, J=6.4 Hz, 1H), 1.83 (dd, J=3.5, 1.2 Hz, 3H), 1.20-1.08 (m, 13H).

³¹P NMR (243 MHz, CDCl₃) δ 149.61, 149.44.

¹³C NMR (151 MHz, CDCl₃) δ 164.24, 164.17, 158.68, 150.94, 150.81, 144.76, 142.47, 142.40, 135.93, 130.20, 130.14, 130.13, 128.27, 128.20, 127.99, 127.02, 126.99, 117.58, 113.29, 113.26, 109.72, 86.34, 86.27, 71.23, 71.14, 70.55, 64.28, 64.04, 58.41, 58.27, 58.14, 55.37, 55.36, 51.81, 51.02, 43.39, 43.31, 43.23, 24.83, 24.78, 24.76, 24.73, 24.70, 24.67, 20.42, 20.38, 20.32, 20.27, 12.34.

### Synthesis of 5′-triazole-PO(OEt)₂-2′OMe-U-CNE

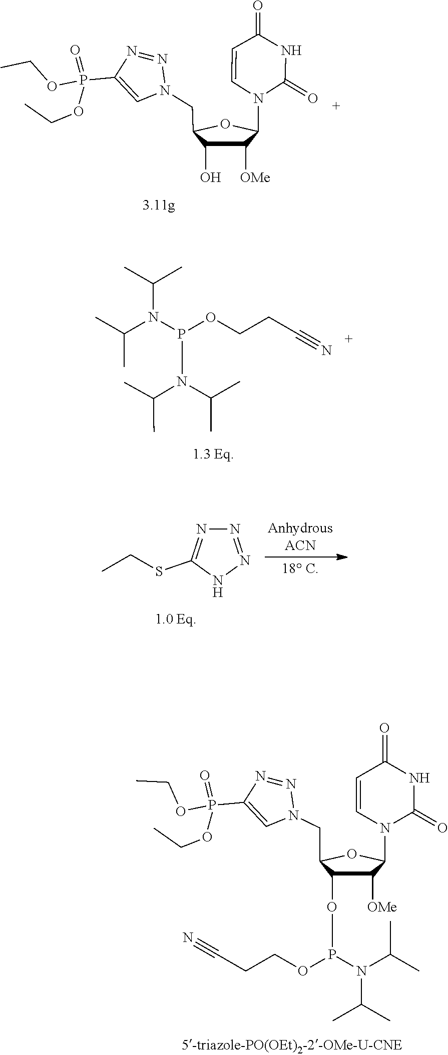

Nucleoside 5′-triazole-PO(OEt)₂-2′OMe-U (1.81 g) was converted to 5′-triazole-PO(OEt)₂-2′OMe-U-CNE-amidite by general procedure (1.82 g, 73% yield) as an off-white solid.

LCMS: C₂₅H₄₁N₇O₉P₂(M−H⁻): 644.50

¹H NMR (600 MHz, CDCl₃) δ 8.92 (s, 1H), 8.19 (s, 1H), 8.16 (s, 1H), 6.91 (d, J=8.1 Hz, 1H), 6.88 (d, J=8.1 Hz, 1H), 5.71 (d, J=8.1 Hz, 1H), 5.68 (d, J=8.1 Hz, 1H), 5.63 (d, J=3.6 Hz, 1H), 5.58 (d, J=3.6 Hz, 1H), 4.94 (dd, J=14.6, 3.1 Hz, 1H), 4.83 (dd, J=14.5, 3.2 Hz, 1H), 4.76 (dd, J=14.6, 6.2 Hz, 1H), 4.69 (dd, J=14.5, 6.8 Hz, 1H), 4.45 (td, J=6.3, 3.1 Hz, 1H), 4.40 (td, J=6.6, 3.2 Hz, 1H), 4.34 (ddt, J=14.2, 9.7, 5.8 Hz, 2H), 4.27-4.14 (m, 8H), 4.11 (dd, J=5.4, 3.6 Hz, 1H), 3.97 (dd, J=5.4, 3.7 Hz, 1H), 3.95-3.90 (m, 2H), 3.81 (ddt, J=10.4, 8.1, 6.1 Hz, 1H), 3.75-3.69 (m, 1H), 3.69-3.60 (m, 4H), 3.50 (s, 3H), 3.47 (s, 2H), 2.84-2.61 (m, 4H), 1.35 (tq, J=7.8, 3.7 Hz, 12H), 1.27 (dd, J=8.2, 6.8 Hz, 2H), 1.23-1.16 (m, 24H).

³¹P NMR (243 MHz, CDCl₃) δ 150.53, 150.42, 14.17, 6.59, 6.54.

¹³C NMR (151 MHz, CDCl₃) δ 162.66, 149.82, 141.50, 140.92, 138.48, 136.90, 132.51, 132.33, 132.11, 118.15, 117.80, 103.11, 102.97, 92.77, 91.68, 81.21, 81.19, 80.93, 80.89, 80.85, 80.78, 71.63, 71.36, 71.27, 63.15, 63.11, 58.82, 58.75, 58.55, 58.43, 57.65, 57.52, 50.90, 50.59, 43.45, 43.36, 24.72, 24.70, 24.67, 24.65, 24.62, 24.56, 20.46, 20.41, 16.32, 16.28, 16.25.

### Synthesis of 2′O-C16-U-CNE

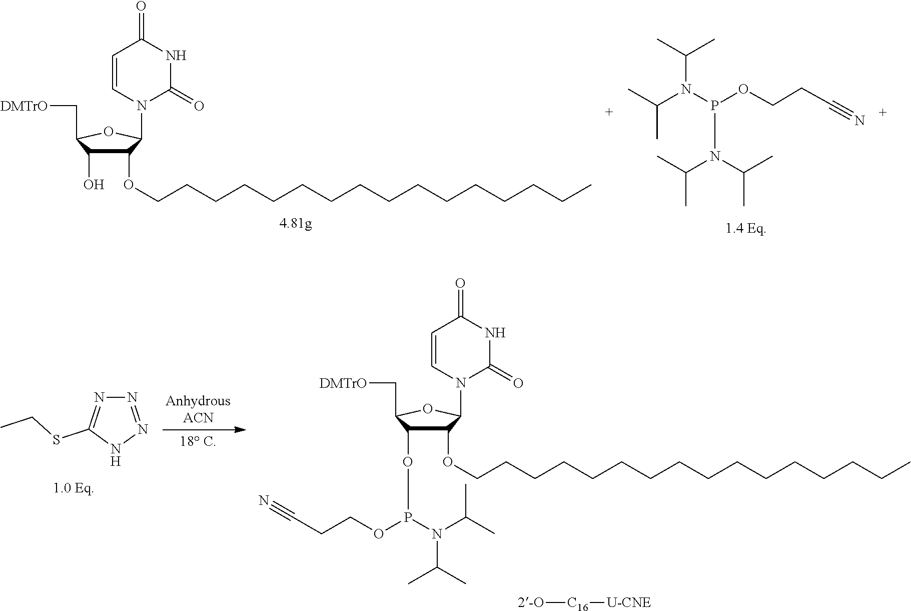

Nucleoside 2′O-C16-U (4.81 g) was converted to 2′O-C16-U-CNE-amidite by general procedure (4.10 g, 68% yield) as an off-white solid.

LCMS: C₅₈H₇₆N₃O₁₁P₂S (M−H⁻). 1052.52

¹H NMR (600 MHz, CDCl₃) δ 8.23 (s, 1H), 8.01 (dd, J=53.9, 8.2 Hz, 1H), 7.39 (dd, J=21.3, 7.7 Hz, 2H), 7.30 (h, J=4.9 Hz, 4H), 7.26 (s, 6H), 6.84 (s, 4H), 5.95 (dd, J=25.5, 2.6 Hz, 1H), 5.21 (t, J=8.2 Hz, 1H), 4.60-4.42 (m, 1H), 4.28-4.18 (m, 1H), 4.00 (ddd, J=12.8, 4.9, 2.5 Hz, 1H), 3.95-3.88 (m, 1H), 3.80 (d, J=3.3 Hz, 6H), 3.78-3.64 (m, 2H), 3.63-3.53 (m, 4H), 3.45 (ddd, J=17.9, 11.1, 2.5 Hz, 1H), 2.63 (q, J=6.4 Hz, 1H), 2.42 (t, J=6.3 Hz, 1H), 1.60 (dhept, J=13.8, 7.1 Hz, 2H), 1.25 (s, 25H), 1.17 (s, 8H), 1.05 (s, 3H), 0.88 (s, 3H).

³¹P NMR (243 MHz, CDCl₃) δ 150.12.

¹³C NMR (151 MHz, CDCl₃) δ 162.95, 162.88, 158.77, 158.74, 140.22, 140.14, 135.26, 135.07, 130.32, 130.30, 130.27, 128.33, 128.29, 127.99, 127.98, 127.23, 113.26, 113.25, 113.23, 102.02, 101.90, 88.17, 87.98, 87.14, 86.96, 82.39, 82.36, 82.16, 82.10, 81.36, 71.19, 70.95, 69.84, 69.74, 61.44, 60.82, 58.56, 58.44, 57.99, 57.86, 55.28, 55.26, 43.34, 43.26, 43.24, 43.16, 31.94, 29.86, 29.84, 29.72, 29.68, 29.66, 29.62, 29.58, 29.54, 29.38, 26.07, 26.06, 24.71, 24.67, 24.62, 24.59, 24.54, 22.71, 20.50, 20.46, 20.32, 20.28, 14.14.

### Example 45: Experimental Procedure for Synthesis of 5′-Bis(Pivaloyloxymethyl)-Triazolyl Phosphonate-2′OMe-Uridine (WV-NU-332)

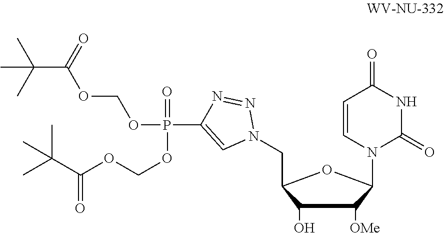

[(((1-(((2R,3R,4R,5R)-5-(2,4-dioxo-3,4-dihydropyrimidin-1(2H)-yl)-3-hydroxy-4-methoxytetrahydrofuran-2-yl)methyl)-1H-1,2,3-triazol-4-yl)phosphoryl)bis(oxy))bis(methylene) bis(2,2-dimethylpropanoate)].

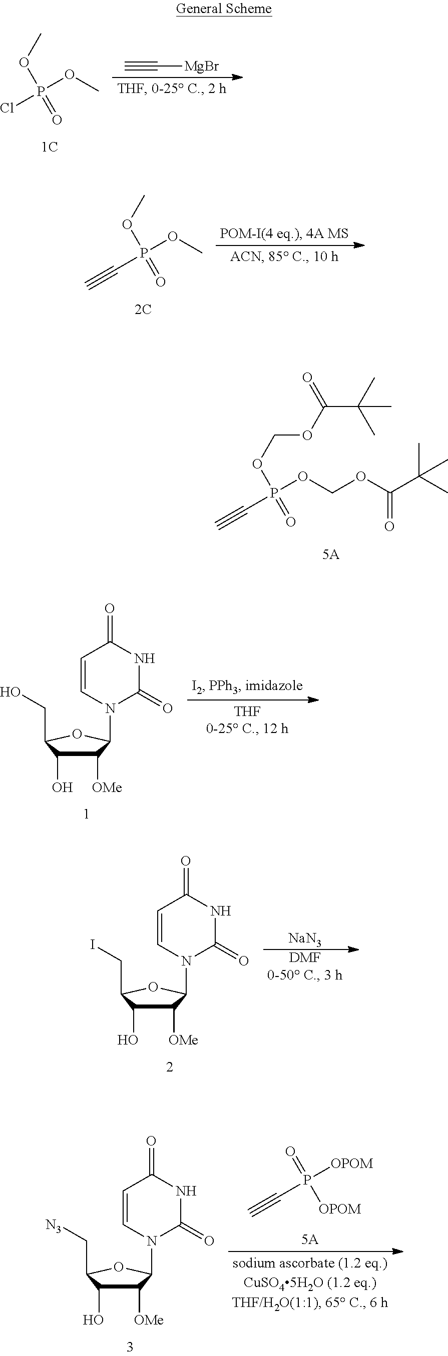

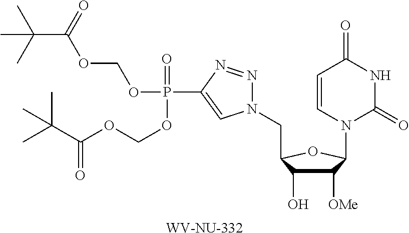

### 1. Preparation of Compound 2C

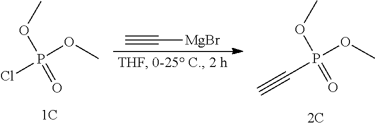

To a solution of compound 1C (10 g, 69.21 mmol, 7.46 mL) in THF (100 mL) was added bromo(ethynyl)magnesium (0.5 M, 166.10 mL) under N₂. The mixture was stirred at 0-25° C. for 2 hr. TLC indicated compound 1C was consumed completely and one new spot formed. The reaction mixture was quenched by addition NH₄Cl 50 mL at 0° C., and then diluted with water 50 mL and extracted with EtOAc (100 mL*3). The combined organic layers were dried over Na₂SO₄, filtered, and concentrated under reduced pressure to give a residue. Without purification. Compound 2C (9 g, crude) was obtained as a yellow oil. TLC: Petroleum ether:Ethyl acetate=0:1, Rf=0.34

### 2. Preparation of Compound 5A

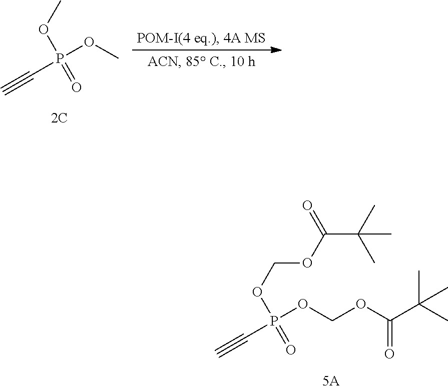

To a solution of compound 2C (9 g, 67.13 mmol) in ACN (200 mL) was added 4A MS (2 g, 67.13 mmol), iodomethyl 2,2-dimethylpropanoate (48.75 g, 201.39 mmol). The mixture was stirred at 82° C. for 10 hr. TLC indicated compound 2C was consumed completely and two new spots formed. The reaction mixture was filtered and concentrated under reduced pressure to give a residue. Compound 5A (5 g, 22.28% yield) was obtained as a colorless oil

¹H NMR (400 MHz, CHLOROFORM-d) 6=5.72 (d, J=1.6 Hz, 2H), 5.69 (d, J=0.9 Hz, 2H), 3.03 (d, J=14.3 Hz, 1H), 1.22 (s, 18H)

³¹P NMR (162 MHz, CHLOROFORM-d) δ=10.31 (s, 1P)

TLC: Petroleum ether:Ethyl acetate=3:1, Rf=0.38

### 3. Preparation of Compound 2

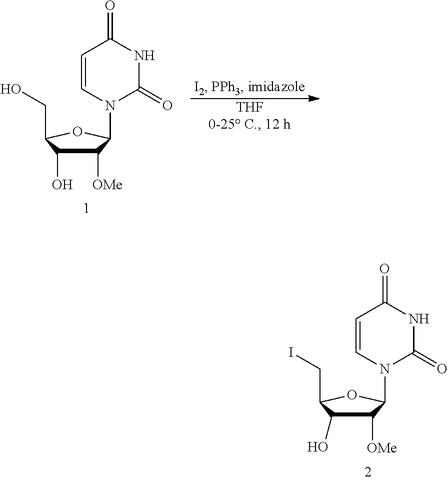

To a solution of compound 1 (50 g, 193.63 mmol) in THF (700 mL) was added imidazole (34.27 g, 503.43 mmol), I₂ (78.63 g, 309.80 mmol) and PPh₃ (81.26 g, 309.80 mmol) at 0° C. The mixture was stirred at 25° C. for 12 hr. LCMS showed compound 1 was consumed completely and the desired mass was detected. Four batches with together. The reaction was quenched by 10% aqueous sodium thiosulfate solution (500 ml). After removing the solvent and volatiles under reduced pressure, the residue was extracted into EtOAc (200 mL*3) and washed with saturated aqueous NaHCO₃ solution. The organic layer was separated, dried over anhydrous Na₂SO₄, filtered and concentrated. The residue was purified by column chromatography (SiO₂, Petroleum ether:Ethyl acetate=1:0 to 0:1). Compound 2 (270 g, 94.73% yield, together with four batches) was obtained as a white solid.

¹H NMR (400 MHz, DMSO-d₆) δ=11.42 (s, 1H), 7.68 (d, J=8.0 Hz, 1H), 5.86 (d, J=5.4 Hz, 1H), 5.69 (d, J=8.0 Hz, 1H), 5.45 (d, J=6.0 Hz, 1H), 4.06-4.01 (m, 1H), 4.00-3.96 (m, 1H), 3.85 (td, J=5.0, 6.2 Hz, 1H), 3.55 (dd, J=5.4, 10.6 Hz, 1H), 3.40 (dd, J=6.9, 10.6 Hz, 1H), 3.34 (s, 3H)

LCMS (M+H+): 368.9

TLC: Petroleum ether:Ethyl acetate=0:1, R_{f}=0.5

### 4. Preparation of Compound 3

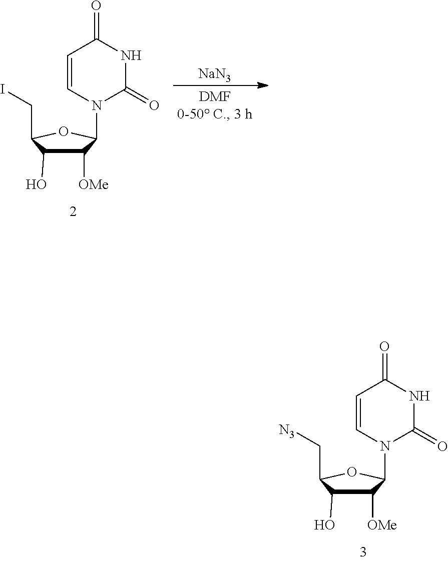

To a solution of compound 2 (10 g, 27.16 mmol) in DMF (100 mL) was added NaN₃ (1.86 g, 28.66 mmol) at 0° C. The mixture was stirred at 0-50° C. for 3 hr. LCMS showed compound 2 was consumed completely and the desired mass was detected. Six batches with together. The reaction was quenched by H₂O (1500 mL), and extracted with Ethyl acetate (500 mL*3). The combined organic layers were washed with saturated aqueous NaCl 100 mL, dried over Na₂SO₄, filtered and concentrated under reduced pressure to give a residue. The residue was purified by column chromatography (SiO₂, Petroleum ether:Ethyl acetate=1:0 to 0:1). The crude product was purified by re-crystallization from DCM (200 mL) at 25° C. Compound 3 (57 g, 93.44% yield, together with six batches) was obtained as a white solid.

¹H NMR (400 MHz, DMSO-d₆) δ=11.41 (br s, 1H), 7.70 (d, J=8.0 Hz, 1H), 5.83 (d, J=4.9 Hz, 1H), 5.68 (d, J=8.0 Hz, 1H), 5.35 (br d, J=6.0 Hz, 1H), 4.07 (q, J=5.3 Hz, 1H), 3.95-3.89 (m, 2H), 3.61 (d, J=4.9 Hz, 2H), 3.36 (s, 3H)

LCMS: (M+H⁺): 284.0, LCMS purity: 100%

TLC: Petroleum ether:Ethyl acetate=0:1, Rf=0.45

### 5. Preparation of Compound WV-NU-332

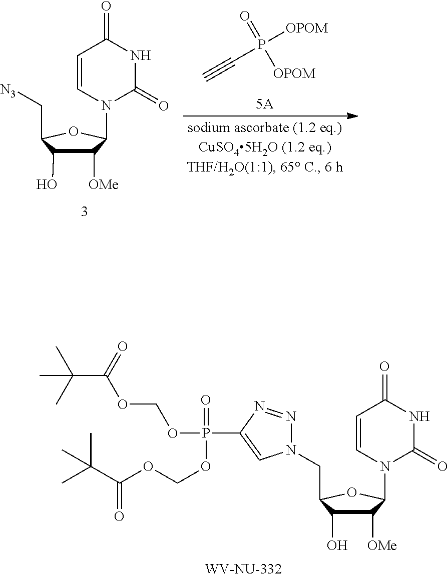

To a solution of compound 3 (3 g, 10.59 mmol) and compound 5A (4.25 g, 12.71 mmol, 1.2 eq) in H₂O (10 mL) was degassed and purged with nitrogen for 3 times, sodium ascorbate (2.52 g, 12.71 mmol, 1.2 eq), diacetoxycopper (2.31 g, 12.71 mmol) was added. The mixture was stirred at 65° C. for 6 hr under N₂ atmosphere. TLC indicated compound 5A was consumed completely and one new spot formed. The reaction mixture was filtered and concentrated under reduced pressure to give a residue. The residue was purified by column chromatography (SiO₂, Petroleum ether:Ethyl acetate=15:1 to 0:1 to Ethyl acetate:MeCN=10:1 to 0:1 to Ethyl acetate:Methanol=8:1). Compound WV-NU-332 (2 g, 46.67% yield, 70% purity) was obtained as a white solid.

¹H NMR (400 MHz, DMSO-d₆) δ=11.47-11.35 (m, 2H), 8.69 (s, 1H), 7.62 (d, J=8.1 Hz, 1H), 5.80 (d, J=5.0 Hz, 1H), 5.70 (s, 2H), 5.67 (s, 2H), 5.51 (d, J=5.8 Hz, 1H), 4.80 (d, J=3.8 Hz, 1H), 4.18-4.15 (m, 1H), 3.96-3.89 (m, 2H), 3.61 (d, J=4.8 Hz, 1H), 3.36 (s, 3H), 1.06 (s, 18H)

³¹P NMR (162 MHz, DMSO-d₆) δ=7.08 (s, 1P)

LCMS: (M+H⁺):618.2, purity:70.8%

TLC: Petroleum ether:Ethyl acetate=0:1, R_{f}=0.06

### Example 46: Synthesis of 5′-DiethylTriazolyl Phosphonate-2′-OMe-Uridine (WV—NU-306)

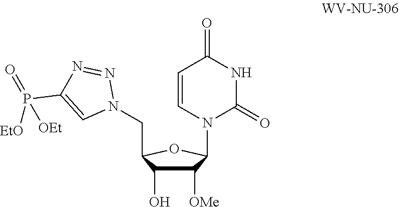

Diethyl(1-(((2R,3R,4R,5R)-5-(2,4-dioxo-3,4-dihydropyrimidin-1(2H)-yl)-3-hydroxy-4-methoxytetrahydrofuran-2-yl)methyl)-1H-1,2,3-triazol-4-yl)phosphonate

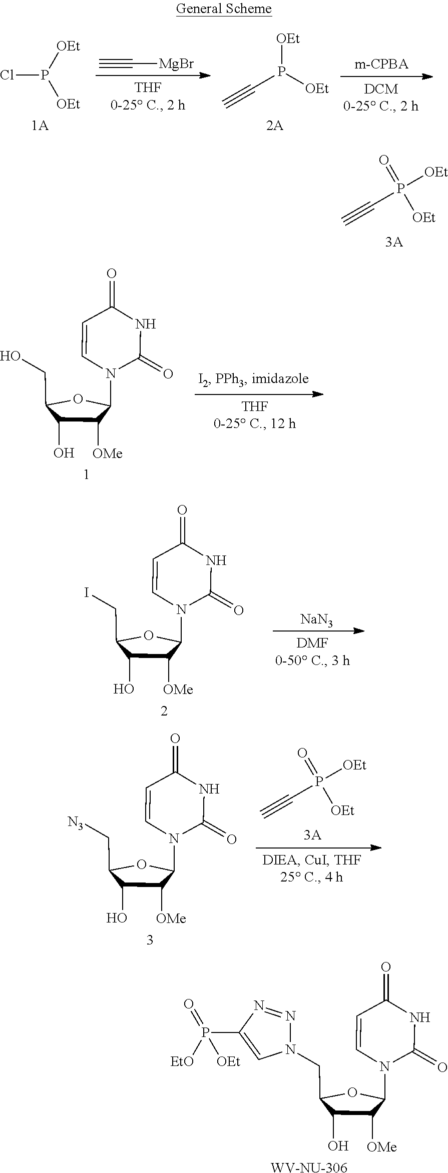

### 1. Preparation of Compound 2A

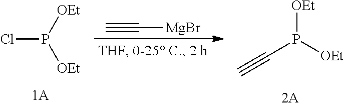

To a solution compound 1A (10 g, 63.88 mmol) in THF (100 mL) was added bromo (ethynyl) magnesium (0.5 M, 124.75 mL) at 0° C. under N₂. The resulting mixture was stirred at 25° C. for 2 hr. TLC indicated compound 1A was consumed completely and two new spots formed. The reaction was clean according to TLC. Four batches with together. The reaction mixture was quenched by sat. aq. NH₄Cl (100 mL) at 0° C., then extracted with DCM (50 mL*3). The combined organic layers were dried over Na₂SO₄, filtered to get the crude. Compound 2A (37.34 g, crude, together with four batches) was obtained as a brown liquid and used into the next step without further purification. TLC: Petroleum ether:Ethyl acetate=2:1, R_{f}=0.25

### 2. Preparation of Compound 3A

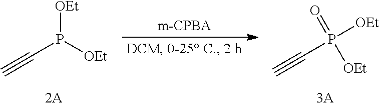

To a solution of compound 2A (37 g, 253.21 mmol) in DCM (1000 mL) was added m-CPBA (102.82 g, 506.42 mmol, 85% purity) at 0° C. The mixture was stirred at 0-25° C. for 2 hr. TLC indicated compound 2A was consumed completely and one new spot formed. The reaction was clean according to TLC. Three batches with together. The reaction mixture was quenched by sat. aq. Na₂SO₃ (300 mL) and NaHCO₃ (300 mL), then extracted with DCM (200 mL*3). The combined organic layers were washed with brine (100 mL*2), dried over Na₂SO₄, filtered, and concentrated under reduced pressure to give a residue. The residue was purified by column chromatography (SiO₂, Petroleum ether: Ethyl acetate=1:0 to 0:1). Compound 3A (55 g, 44.66% yield, together with three batches) was obtained as a colorless oil.

¹H NMR (400 MHz, CHLOROFORM-d) 6=4.16-4.07 (m, 4H), 2.97 (d, J=13.3 Hz, 1H), 1.31 (dt, J=0.7, 7.1 Hz, 6H)

³¹P NMR (162 MHz, CHLOROFORM-d) 6=−8.43 (s, 1P)

TLC: Petroleum ether:Ethyl acetate=1:1, R_{f}=0.4

### 3. Preparation of Compound 2

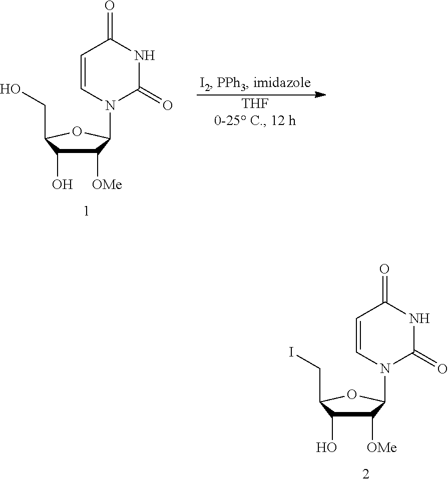

To a solution of compound 1 (50 g, 193.63 mmol) in THF (700 mL) was added imidazole (34.27 g, 503.43 mmol), I₂ (78.63 g, 309.80 mmol) and PPh₃ (81.26 g, 309.80 mmol) at 0° C. The mixture was stirred at 25° C. for 12 hr. LCMS showed compound 1 was consumed completely and the desired mass was detected. Four batches with together. The reaction was quenched by 10% aqueous sodium thiosulfate solution (500 ml). After removing the solvent and volatiles under reduced pressure, the residue was extracted into EtOAc (200 mL*3) and washed with saturated aqueous NaHCO₃ solution. The organic layer was separated, dried over anhydrous Na₂SO₄, filtered and concentrated. The residue was purified by column chromatography (SiO₂, Petroleum ether:Ethyl acetate=1:0 to 0:1). Compound 2 (270 g, 94.73% yield, together with four batches) was obtained as a white solid.

¹H NMR (400 MHz, DMSO-d₆) δ=11.42 (s, 1H), 7.68 (d, J=8.0 Hz, 1H), 5.86 (d, J=5.4 Hz, 1H), 5.69 (d, J=8.0 Hz, 1H), 5.45 (d, J=6.0 Hz, 1H), 4.06-4.01 (m, 1H), 4.00-3.96 (m, 1H), 3.85 (td, J=5.0, 6.2 Hz, 1H), 3.55 (dd, J=5.4, 10.6 Hz, 1H), 3.40 (dd, J=6.9, 10.6 Hz, 1H), 3.34 (s, 3H)

LCMS (M+H+): 368.9

TLC: Petroleum ether:Ethyl acetate=0:1, R_{f}=0.5

### 4. Preparation of Compound 3

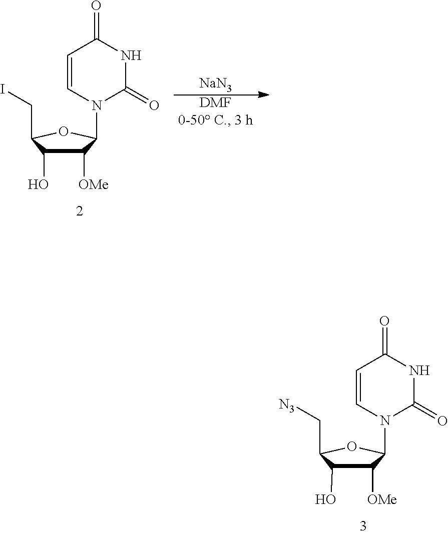

To a solution of compound 2 (10 g, 27.16 mmol) in DMF (100 mL) was added NaN₃ (1.86 g, 28.66 mmol) at 0° C. The mixture was stirred at 0-50° C. for 3 hr. LCMS showed compound 2 was consumed completely and the desired mass was detected. Six batches with together. The reaction was quenched by H₂O (1500 mL), and extracted with Ethyl acetate (500 mL*3). The combined organic layers were washed with saturated aqueous NaCl 100 mL, dried over Na₂SO₄, filtered and concentrated under reduced pressure to give a residue. The residue was purified by column chromatography (SiO₂, Petroleum ether: Ethyl acetate=1:0 to 0:1). The crude product was purified by re-crystallization from DCM (200 mL) at 25° C. Compound 3 (57 g, 93.44% yield, together with six batches) was obtained as a white solid.

¹H NMR (400 MHz, DMSO-d₆) δ=11.41 (br s, 1H), 7.70 (d, J=8.0 Hz, 1H), 5.83 (d, J=4.9 Hz, 1H), 5.68 (d, J=8.0 Hz, 1H), 5.35 (br d, J=6.0 Hz, 1H), 4.07 (q, J=5.3 Hz, 1H), 3.95-3.89 (m, 2H), 3.61 (d, J=4.9 Hz, 2H), 3.36 (s, 3H)

LCMS: (M+H⁺): 284.0, LCMS purity: 100% TLC: Petroleum ether:Ethyl acetate=0:1, R_{f}=0.45

### 5. Preparation of WV-NU-306

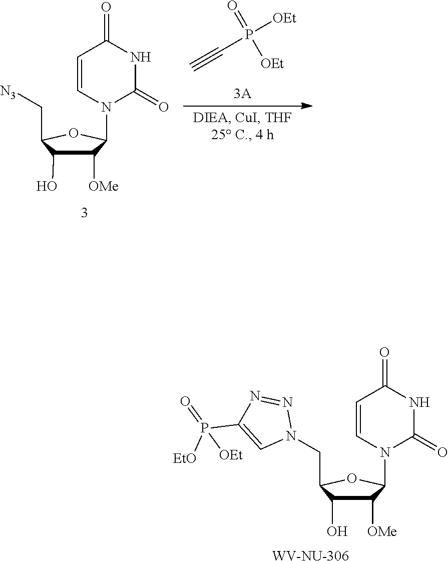

To a solution of compound 3 (9.31 g, 57.42 mmol) and compound 3A (9.31 g, 57.42 mmol) in THF (140 mL) was degassed and purged with N₂ for 3 times, then DIEA (12.69 g, 98.15 mmol), CuI (18.69 g, 98.15 mmol) was added. The mixture was stirred at 25° C. for 4 hr under N₂ atmosphere. LCMS showed compound 3 was consumed completely and the desired mass was detected. Two batches with together. The reaction mixture was concentrated under reduced pressure to give product. The residue was purified by column chromatography (SiO₂, Petroleum ether:Acetonitrile=1:0 to 0:1 to Dichloromethane:Methanol=1:0 to 0:1). Compound WV-NU-306 (56 g, 62.92% yield, together with two batches) was obtained as a yellow solid.

### Batch 2 (46.43 g):

¹H NMR (400 MHz, CHLOROFORM-d) 6=9.79 (br s, 1H), 8.27 (s, 1H), 7.03 (d, J=8.0 Hz, 1H), 5.72 (d, J=8.0 Hz, 1H), 5.62 (d, J=2.4 Hz, 1H), 4.96-4.70 (m, 2H), 4.30-4.12 (m, 6H), 3.97 (dd, J=2.4, 4.9 Hz, 1H), 3.55 (s, 3H), 3.48 (s, 1H), 1.35 (t, J=7.0 Hz, 6H)

³¹P NMR (162 MHz, CHLOROFORM-d) δ=6.69 (s, 1P)

LCMS (M+H⁺):446.1, purity: 97.42%; TLC: DCM:MeOH=10:1, R_{f}=0.65

### Batch 3 (9.22 g):

¹H NMR (400 MHz, CHLOROFORM-d) 6=9.54 (s, 1H), 8.27 (s, 1H), 7.01 (d, J=8.0 Hz, 1H), 5.73 (dd, J=1.6, 8.0 Hz, 1H), 5.62 (d, J=2.3 Hz, 1H), 4.95-4.88 (m, 1H), 4.75 (dd, J=5.6, 14.4 Hz, 1H), 4.30-4.15 (m, 6H), 3.97 (dd, J=2.2, 4.8 Hz, 1H), 3.56 (s, 3H), 3.52 (br d, J=6.6 Hz, 1H), 1.36 (t, J=7.0 Hz, 6H)

³¹P NMR (162 MHz, CHLOROFORM-d) δ=6.64 (s, 1P)

LCMS (M+H⁺): 446.1, purity: 93.76%

TLC: DCM:MeOH=10:1, R_{f}=0.65

### Example 47: Synthesis of 5′-Bis(2-cyanoethyl)-Triazolyl Phosphonate-2′-OMe-Uridine (WV-NU-336)

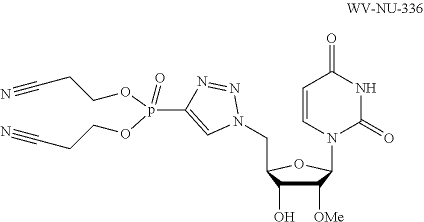

Bis(2-cyanoethyl) (1-(((2R,3R,4R,5R)-5-(2,4-dioxo-3,4-dihydropyrimidin-1(2H)-yl)-3-hydroxy-4-methoxytetrahydrofuran-2-yl)methyl)-1H-1,2,3-triazol-4-yl)phosphonate

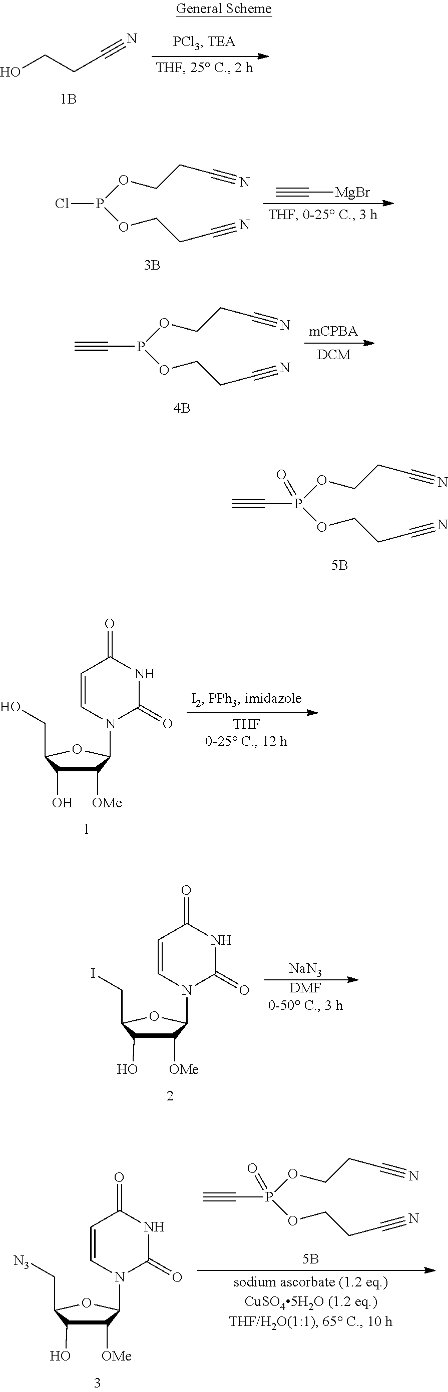

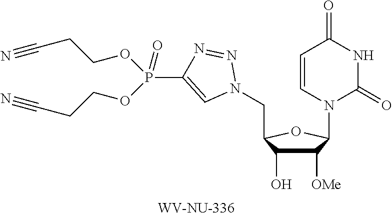

### 1. Preparation of Compound 3B

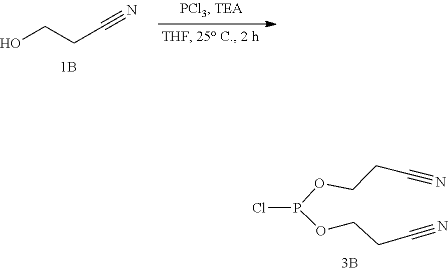 [SMILES: N#CCCO ; N#CCCOP(Cl)OCCC#N]

To a solution of PCl₃ (35 g, 254.86 mmol) in THF (1000 mL) was added TEA (51.58 g, 509.71 mmol, 70.95 mL) and 3-hydroxypropanenitrile (36.23 g, 509.71 mmol, 34.67 mL). The mixture was stirred at 25° C. for 2 hr. TLC indicated compound 1B was consumed completely and one new spot formed. The reaction mixture was, filtered and concentrated under reduced pressure to give a residue. Without purification. Compound 3B (44 g, 83.58% yield) was obtained as a colorless oil.

TLC: Petroleum ether:Ethyl acetate=0:1, Rf=0.12

### 2. Preparation of Compound 4B

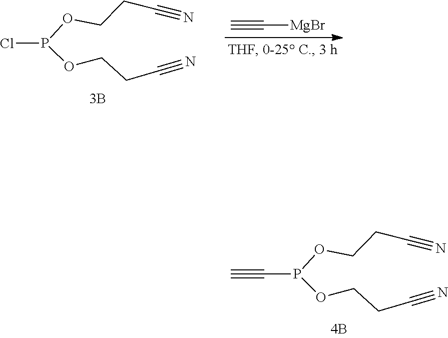

To a solution of compound 3B (10 g, 48.41 mmol) in THF (100 mL) was added bromo(ethynyl)magnesium (0.5 M, 116.19 mL) at 0° C. The mixture was stirred at 0-25° C. for 5 hr. TLC indicated compound 3B was consumed completely and two new spots formed. The reaction mixture was quenched by addition NH₄Cl 50 mL at 0° C., and then diluted with water 100 mL and extracted with EtOAc (100 mL*3). The combined organic layers were dried over Na₂SO₄, filtered, and concentrated under reduced pressure to give a residue. Compound 4B (6 g, 63.19% yield) was obtained as a yellow oil.

¹H NMR (400 MHz, CHLOROFORM-d) 6=4.17-4.09 (m, 4H), 3.19 (d, J=2.1 Hz, 1H), 2.67 (t, J=6.1 Hz, 4H)

³¹P NMR (162 MHz, CHLOROFORM-d) 6=132.04 (s, 1P)

### For the Scale Up Batch:

To a solution of compound 3B (44 g, 213.01 mmol) in THF (1000 mL) was added bromo(ethynyl)magnesium (0.5 M, 511.22 mL) at 0° C. The mixture was stirred at 0-25° C. for 3 hr. TLC indicated compound 3B was consumed completely and two new spots formed. The reaction mixture was quenched by sat. NH₄Cl (200 mL) at 0° C., then extracted with DCM (500 mL*3). The combined organic layers were dried over Na₂SO₄, filtered to get the crude. No purification. Compound 4B (40 g, crude) was obtained as a yellow oil.

TLC: Petroleum ether:Ethyl acetate=1:1, Rf=0.39

### 3. Preparation of Compound 5B

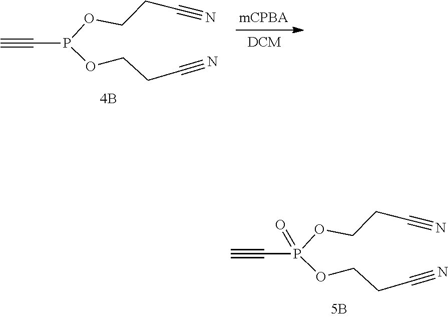

To a solution of compound 4B (40 g, 203.93 mmol) in DCM (1000 mL) was added mCPBA (62.10 g, 305.90 mmol, 85% purity). The mixture was stirred at 0-25° C. for 2 hr. TLC indicated compound 4B was consumed completely and one new spot formed. The reaction mixture was quenched by sat. Na₂SO₃ (2000 mL) and NaHCO₃ (2000 mL), then extracted with DCM (1000 mL*2). The combined organic layers were washed with brine (500 ml), dried over Na₂SO₄, filtered, and concentrated under reduced pressure to give a residue. The residue was purified by column chromatography (SiO₂, Petroleum ether:Ethyl acetate=10:1 to 0:1). Compound 5 B (13 g, 30.05% yield) was obtained as a yellow oil.

¹H NMR (400 MHz, CHLOROFORM-d) 6=4.37-4.29 (m, 4H), 3.16 (dd, J=1.3, 13.9 Hz, 1H), 2.81 (t, J=6.1 Hz, 4H)

³¹P NMR (162 MHz, CHLOROFORM-d) δ=−8.61 (s, 1P)

TLC: Petroleum ether:Ethyl acetate=1:1, R_{f}=0.23

### 4. Preparation of Compound 2

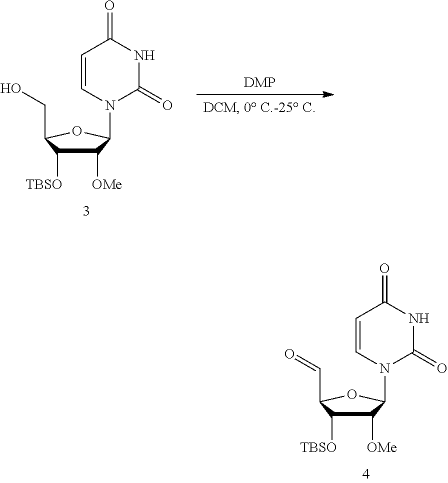

To a solution of compound 1 (50 g, 193.63 mmol) in THF (700 mL) was added imidazole (34.27 g, 503.43 mmol), 1₂ (78.63 g, 309.80 mmol) and PPh₃ (81.26 g, 309.80 mmol) at 0° C. The mixture was stirred at 25° C. for 12 hr. LCMS showed compound 1 was consumed completely and the desired mass was detected. Four batches with together. The reaction was quenched by 10% aqueous sodium thiosulfate solution (500 ml). After removing the solvent and volatiles under reduced pressure, the residue was extracted into EtOAc (200 mL*3) and washed with saturated aqueous NaHCO₃ solution. The organic layer was separated, dried over anhydrous Na₂SO₄, filtered and concentrated. The residue was purified by column chromatography (SiO₂, Petroleum ether:Ethyl acetate=1:0 to 0:1). Compound 2 (270 g, 94.73% yield, together with four batches) was obtained as a white solid.

¹H NMR (400 MHz, DMSO-d₆) δ=11.42 (s, 1H), 7.68 (d, J=8.0 Hz, 1H), 5.86 (d, J=5.4 Hz, 1H), 5.69 (d, J=8.0 Hz, 1H), 5.45 (d, J=6.0 Hz, 1H), 4.06-4.01 (m, 1H), 4.00-3.96 (m, 1H), 3.85 (td, J=5.0, 6.2 Hz, 1H), 3.55 (dd, J=5.4, 10.6 Hz, 1H), 3.40 (dd, J=6.9, 10.6 Hz, 1H), 3.34 (s, 3H)

LCMS (M+H+): 368.9

TLC: Petroleum ether:Ethyl acetate=0:1, Rf=0.5

### 5. Preparation of Compound 3

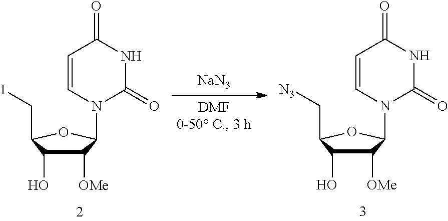

For six batches. To a solution of compound 2 (10 g, 27.16 mmol) in DMF (100 mL) was added NaN₃ (1.86 g, 28.66 mmol) at 0° C. The mixture was stirred at 0-50° C. for 3 hr. LCMS showed compound 2 was consumed completely and the desired mass was detected. Six batches with together. The reaction was quenched by H₂O (1500 mL), and extracted with Ethyl acetate (500 mL*3). The combined organic layers were washed with saturated aqueous NaCl 100 mL, dried over Na₂SO₄, filtered and concentrated under reduced pressure to give a residue. The residue was purified by column chromatography (SiO₂, Petroleum ether:Ethyl acetate=1:0 to 0:1). The crude product was purified by re-crystallization from DCM (200 mL) at 25° C. Compound 3 (57 g, 93.44% yield) was obtained as a white solid.

¹H NMR (400 MHz, DMSO-d₆) δ=11.41 (br s, 1H), 7.70 (d, J=8.0 Hz, 1H), 5.83 (d, J=4.9 Hz, 1H), 5.68 (d, J=8.0 Hz, 1H), 5.35 (br d, J=6.0 Hz, 1H), 4.07 (q, J=5.3 Hz, 1H), 3.95-3.89 (m, 2H), 3.61 (d, J=4.9 Hz, 2H), 3.36 (s, 3H)

LCMS: (M+H⁺): 284.0, LCMS purity: 100%

TLC: Petroleum ether:Ethyl acetate=0:1, Rf=0.45

### 6. Preparation of Compound WV-NU-336

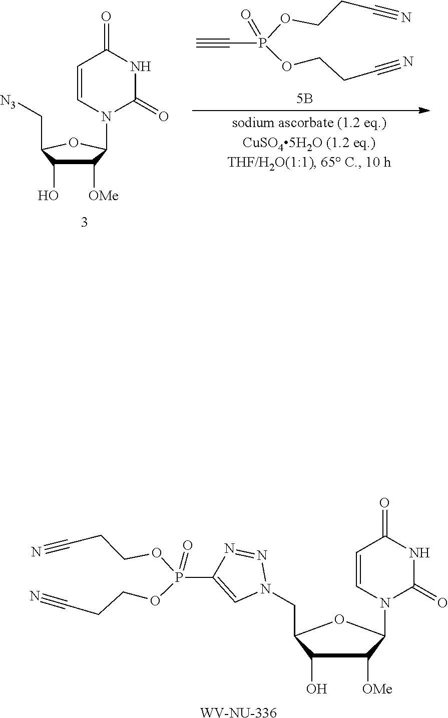

To a solution of compound 3 (5 g, 17.65 mmol) and compound 5B (4.49 g, 21.18 mmol) in THF (10 mL) and H₂O (10 mL) was degassed and purged with N₂ for 3 times, then CuSO₄·5H₂O (5.29 g, 21.18 mmol), sodium ascorbate (4.20 g, 21.18 mmol) was added. The mixture was stirred at 65° C. for 10 hr under N₂ atmosphere. LCMS showed compound 3 was consumed completely and desired mass was detected. The reaction mixture was filtered and concentrated under reduced pressure to give a residue. The residue was purified by column chromatography (SiO₂, Petroleum ether:Ethyl acetate=10:1 to 0:1, Ethyl acetate MeCN=8:1 to 0:1). Compound WV-NU-336 (5.3 g, 60.61% yield) was obtained as a white solid.

¹H NMR (400 MHz, DMSO-d₆) δ=11.40 (d, J=1.6 Hz, 1H), 8.72 (s, 1H), 7.60 (d, J=8.0 Hz, 1H), 5.79 (d, J=4.8 Hz, 1H), 5.66 (dd, J=2.1, 8.0 Hz, 1H), 5.50 (d, J=6.1 Hz, 1H), 4.87-4.73 (m, 2H), 4.32-4.17 (m, 5H), 4.14 (q, J=5.5 Hz, 1H), 3.93 (t, J=5.0 Hz, 1H), 3.36 (s, 3H), 2.95 (t, J=5.9 Hz, 4H)

³¹P NMR (162 MHz, DMSO-d6) δ=7.72 (s, 1P)

LCMS (M+H⁺): 496.1, purity: 93.49%

TLC: Dichloromethane:Methanol=8:1, Rf=0.13

### Example 48: Synthesis of Conjugated Free Amine Oligonucleotide Lipid/Ligands

### General Procedure for Free Amine Oligonucleotide Lipid/Ligand Conjugation

A stock solution of 5′-amino oligo (SSR-0106564) was made by dissolving in 2:1 DMSO/water (1 mg/15 μL). A stock solution of HATU was made by dissolving in NMP (1 mg/50 μL). To a solution of conjugate in NMP (0.075M) was added DIPEA (2.5 eq.) and HATU (0.75 eq.). The ligand mixture was stirred at room temperature for 30 minutes. The conjugate solution (4 eq.) was added into the solution of SSR-0106564 (1 eq.). The reaction mixture was stirred at room temperature and monitored by UPLC-MS. After disappearance of starting material, reaction mixture purified by HPLC.

### Example 49: Synthesis of D-DPSE and L-DPSE Amidites

### General Procedure for Synthesis of D-DPSE and L-DPSE Amidites

Nucleosides (1.0 eq.) in an appropriate size three necked flask was azeotroped with anhydrous toluene (15 mL/g) and anhydrous acetonitrile (15 mL/g) and was dried for 24 hours on high vacuum. To the flask was added anhydrous THF (0.2 M solution) under argon and solution was cooled to −5° C. To the reaction mixture was added triethyl amine (5.0 eq.) followed by addition of D-DPSE-Cl (1.25 M) or L-DPSE-Cl (0.9M) solution (1.8-2.2 eq.) over the period of 5-10 min. The reaction mixture was warmed to room temperature and reaction progress was monitored by HPLC. After disappearance of starting material, the reaction mixture was filtered through fritted glass tube. Reaction flask and precipitate was washed with anhydrous THF. Obtained filtrate was collected and solvent was removed under reduced pressure. The residue was purified by column chromatography (SiO2, 40-100% Ethyl acetate in Hexanes with 5% triethyl amine) to give NU-D-DPSE or NU-L-DPSE Amidite as off-white solid.

### Synthesis of 5′-triazole-PO(OEt)₂-2′OMe-U-L-DPSE

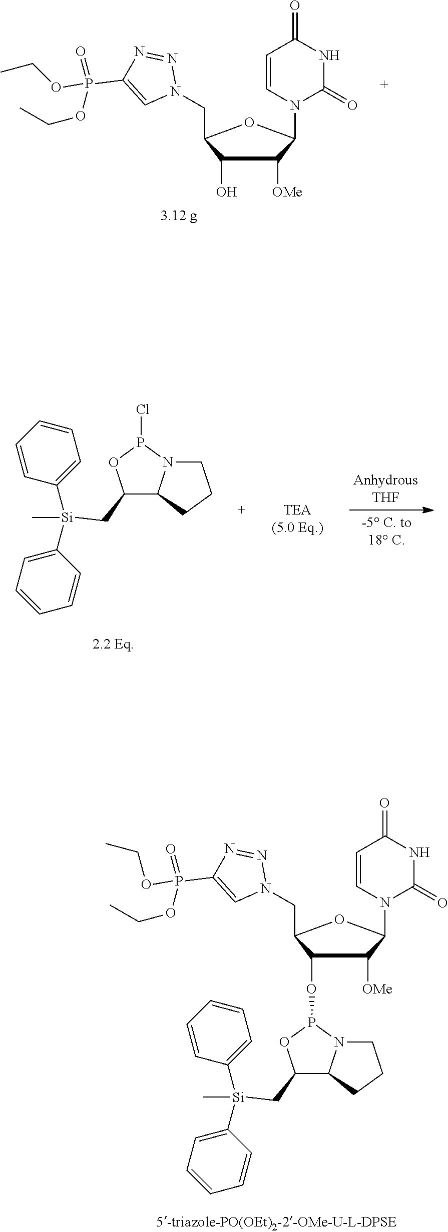

Nucleoside 5′-triazole-PO(OEt)₂-2′OMe-U (3.12 g) was converted to 5′-triazole-PO(OEt)₂-2′OMe-U-L-DPSE-amidite by general procedure (3.18 g, 60% yield) as an off-white solid.

LCMS: C₃₅H₄₆N₆O₉P₂Si (M−H⁻): 783.36

¹H NMR (600 MHz, CDCl₃) δ 8.70 (s, 1H), 8.07 (s, 1H), 7.54 (d, J=7.6 Hz, 2H), 7.50 (d, J=7.7 Hz, 2H), 7.32 (s, 7H), 6.82 (d, J=8.1 Hz, 1H), 5.70 (d, J=8.1 Hz, 1H), 5.37 (d, J=3.1 Hz, 1H), 4.92 (d, J=8.3 Hz, 1H), 4.61 (dd, J=14.6, 2.9 Hz, 1H), 4.44 (s, 2H), 4.20 (s, 5H), 3.79 (d, J=2.4 Hz, 1H), 3.59 (dd, J=7.0, 3.4 Hz, 1H), 3.50 (d, J=2.8 Hz, 1H), 3.38 (s, 3H), 3.21 (dd, J=9.0, 4.4 Hz, 1H), 1.89 (dd, J=8.1, 3.5 Hz, 1H), 1.75 (d, J=9.6 Hz, 1H), 1.65 (dd, J=14.7, 8.5 Hz, 1H), 1.47 (d, J=6.2 Hz, 2H), 1.36 (d, J=5.0 Hz, 6H), 0.67 (s, 3H).

³¹P NMR (243 MHz, CDCl₃) δ 152.69, 6.72

¹³C NMR (151 MHz, CDCl₃) δ 162.85, 149.56, 141.88, 136.59, 135.94, 134.57, 134.36, 132.29, 129.49, 129.43, 127.99, 127.94, 102.90, 93.49, 81.01, 80.59, 79.58, 77.11, 76.90, 76.69, 70.73, 70.67, 67.90, 63.12, 63.08, 58.93, 50.34, 46.74, 46.51, 27.26, 27.21, 25.93, 25.91, 17.75, 16.32, 16.28, −3.35, −3.39.

### Synthesis of 5′-triazole-PO(OEt)₂-2′OMe-U-D-DPSE

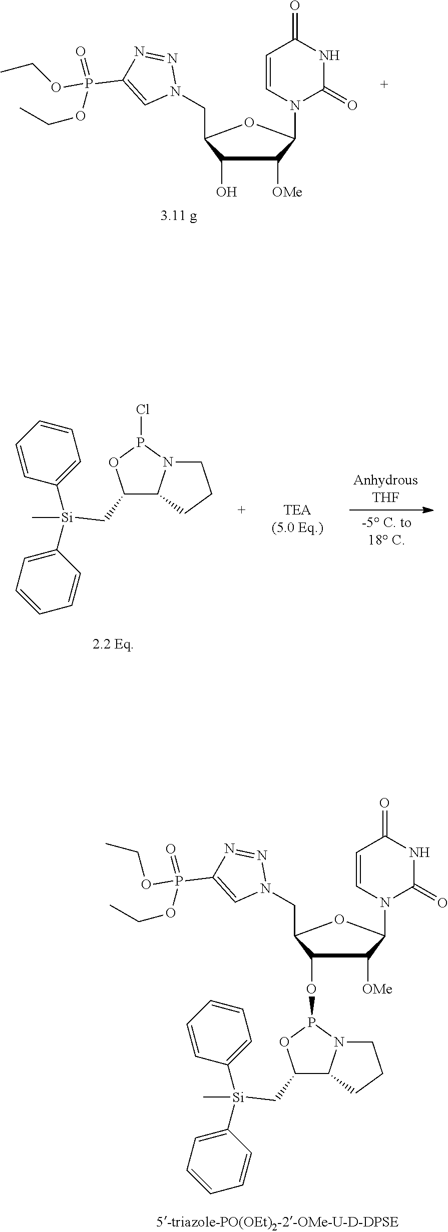

Nucleoside 5′-triazole-PO(OEt)₂-2′OMe-U (3.11 g) was converted to 5′-triazole-PO(OEt)₂-2′OMe-U-D-DPSE-amidite by general procedure (3.35 g, 63% yield) as an off-white solid.

LCMS: C₃₅H₄₆N₆O₉P₂Si (M−H⁻): 783.36

¹H NMR (600 MHz, CDCl₃) δ 9.33 (s, 1H), 8.10 (s, 1H), 7.53 (ddt, J=17.2, 6.5, 1.6 Hz, 4H), 7.33 (dtdd, J=11.0, 8.5, 3.9, 1.9 Hz, 6H), 6.82 (d, J=8.1 Hz, 1H), 5.70 (d, J=8.1 Hz, 1H), 5.47 (d, J=3.3 Hz, 1H), 4.92 (td, J=7.4, 5.4 Hz, 1H), 4.70 (dd, J=14.5, 3.1 Hz, 1H), 4.45 (dd, J=14.6, 6.8 Hz, 1H), 4.37 (ddd, J=9.4, 6.7, 5.6 Hz, 1H), 4.30-4.15 (m, 5H), 3.85 (dd, J=5.6, 3.4 Hz, 1H), 3.59 (ddt, J=14.5, 10.6, 7.5 Hz, 1H), 3.50-3.44 (m, 1H), 3.26 (s, 3H), 3.17 (tdd, J=10.8, 8.7, 4.6 Hz, 1H), 1.85 (dtq, J=12.4, 8.1, 3.6 Hz, 1H), 1.71 (dtd, J=15.0, 8.3, 4.5 Hz, 1H), 1.56 (ddd, J=88.8, 14.6, 7.5 Hz, 2H), 1.45-1.37 (m, 1H), 1.35 (td, J=7.0, 5.7 Hz, 6H), 1.29 (dt, J=9.8, 8.2 Hz, 1H), 0.67 (s, 3H).

³¹P NMR (243 MHz, CDCl₃) δ 154.12, 6.58

¹³C NMR (151 MHz, CDCl₃) δ 162.90, 149.72, 141.36, 136.44, 136.05, 134.61, 134.45, 132.33, 132.11, 129.53, 129.48, 127.99, 127.96, 102.96, 92.53, 81.07, 81.06, 80.62, 80.59, 79.60, 79.53, 70.72, 70.65, 67.50, 67.48, 63.16, 63.12, 58.58, 50.50, 46.93, 46.69, 27.15, 26.02, 26.00, 17.92, 17.89, 16.33, 16.31, 16.29, 16.27, −3.27.

### Synthesis of 5′-vinyl-PO(OEt)₂-2′OMe-U-L-DPSE

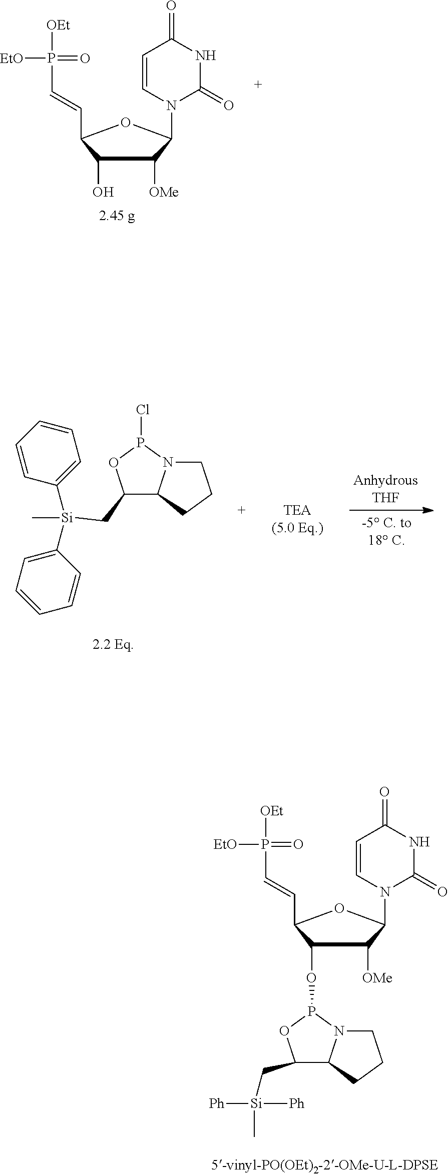

Nucleoside 5′-vinyl-PO(OEt)₂-2′OMe-U (2.45 g) was converted to 5′-vinyl-PO(OEt)₂-2′OMe-U-L-DPSE-amidite by general procedure (2.58 g, 56% yield) as an off-white solid.

LCMS: C₃₄H₄₅N₃O₉P₂Si (M−H⁻). 728.48

¹H NMR (600 MHz, CDCl₃) δ 8.47 (s, 1H), 7.55-7.50 (m, 4H), 7.40-7.31 (m, 6H), 7.18 (d, J=8.1 Hz, 1H), 6.79 (ddd, J=22.0, 17.2, 4.7 Hz, 1H), 5.99 (ddd, J=19.1, 17.2, 1.7 Hz, 1H), 5.76-5.73 (m, 2H), 4.86 (dt, J=8.1, 6.2 Hz, 1H), 4.43 (dddd, J=6.4, 4.7, 3.1, 1.8 Hz, 1H), 4.26 (ddd, J=8.8, 6.4, 5.2 Hz, 1H), 4.14-4.06 (m, 4H), 3.73 (dd, J=5.2, 3.7 Hz, 1H), 3.54 (dddd, J=14.6, 10.6, 8.1, 6.9 Hz, 1H), 3.41 (s, 4H), 3.18 (tdd, J=10.8, 8.8, 4.5 Hz, 1H), 1.85 (dtq, J=12.4, 8.1, 4.1, 3.5 Hz, 1H), 1.73-1.61 (m, 2H), 1.46 (dd, J=14.6, 6.6 Hz, 1H), 1.43-1.36 (m, 1H), 1.33 (td, J=7.1, 4.7 Hz, 6H), 1.28-1.22 (m, 1H), 0.66 (s, 3H).

³¹P NMR (243 MHz, CDCl₃) δ 152.97, 16.88

¹³C NMR (151 MHz, CDCl₃) δ 162.70, 149.59, 146.68, 146.65, 140.33, 136.48, 135.99, 134.54, 134.39, 129.56, 129.49, 128.01, 127.97, 120.08, 118.84, 102.79, 90.28, 81.72, 81.70, 79.58, 79.51, 73.20, 73.14, 67.73, 67.71, 62.06, 62.02, 61.99, 58.72, 58.71, 46.66, 46.42, 27.17, 25.93, 25.91, 17.80, 17.77, 16.46, 16.41, −3.38.

### Synthesis of 5′-vinyl-PO(OEt)₂-2′OMe-U-D-DPSE

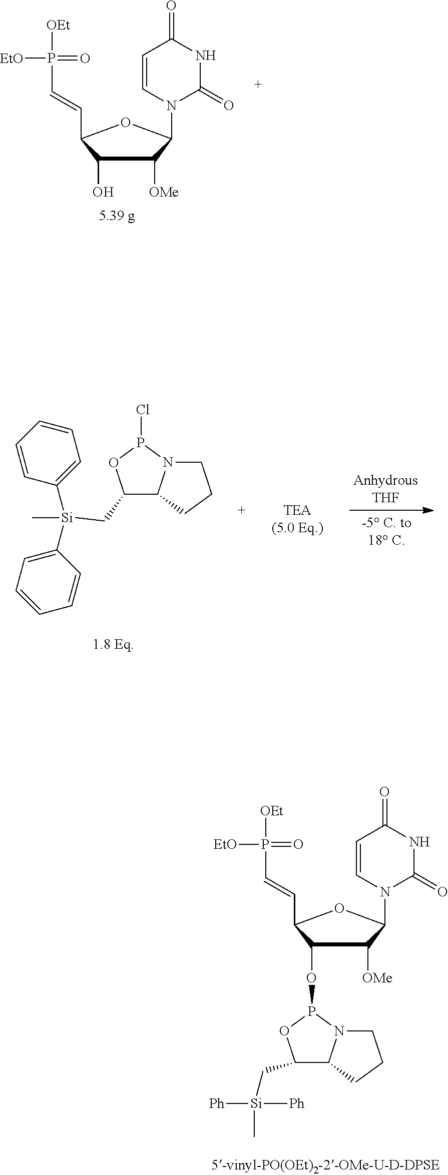

Nucleoside 5′-vinyl-PO(OEt)₂-2′OMe-U (5.39 g) was converted to 5′-vinyl-PO(OEt)₂-2′OMe-U-D-DPSE-amidite by general procedure (8.60 g, 85% yield) as an off-white solid.

LCMS: C₃₄H₄₅N₃O₉P₂Si (M−H⁻): 728.29

¹H NMR (600 MHz, CDCl₃) δ 8.98 (s, 1H), 7.52 (ddt, J=8.2, 6.4, 1.6 Hz, 4H), 7.35 (dddd, J=13.7, 8.3, 7.0, 3.9 Hz, 6H), 7.24 (d, J=8.2 Hz, 1H), 6.82 (ddd, J=22.1, 17.2, 4.9 Hz, 1H), 6.01 (ddd, J=18.9, 17.2, 1.7 Hz, 1H), 5.82 (d, J=2.5 Hz, 1H), 5.75 (d, J=8.2 Hz, 1H), 4.89 (td, J=7.5, 5.3 Hz, 1H), 4.59-4.53 (m, 1H), 4.16-4.04 (m, 5H), 3.67 (dd, J=5.1, 2.5 Hz, 1H), 3.53 (ddt, J=14.8, 10.6, 7.6 Hz, 1H), 3.40 (ddt, J=9.5, 7.2, 5.4 Hz, 1H), 3.26 (s, 3H), 3.17 (tdd, J=10.8, 8.8, 4.3 Hz, 1H), 1.87-1.78 (m, 1H), 1.67 (dd, J=14.6, 7.2 Hz, 2H), 1.49 (dd, J=14.5, 7.8 Hz, 1H), 1.33 (q, J=7.0 Hz, 7H), 1.27-1.18 (m, 2H), 0.65 (s, 3H).

³¹P NMR (243 MHz, CDCl₃) δ 152.43, 16.64

¹³C NMR (151 MHz, CDCl₃) δ 161.50, 148.55, 145.48, 145.44, 138.46, 135.24, 134.98, 133.51, 133.41, 128.59, 128.52, 127.00, 126.96, 119.66, 118.41, 101.69, 88.59, 81.37, 77.98, 77.92, 72.29, 72.20, 66.40, 66.38, 61.05, 61.01, 57.44, 45.86, 45.63, 25.95, 24.86, 24.84, 16.92, 16.89, 15.41, 15.37, −4.41.

### Example 50: Synthesis and Analysis of Homo-DNA and Amidite PNs

### General Structure for Sugar and Base Modifications

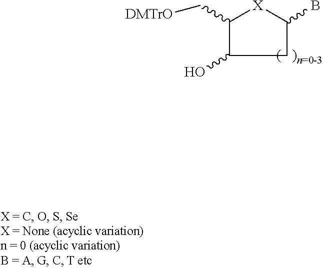

### Synthesis of WV-NU-223

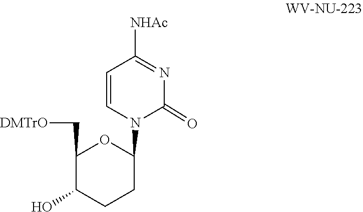

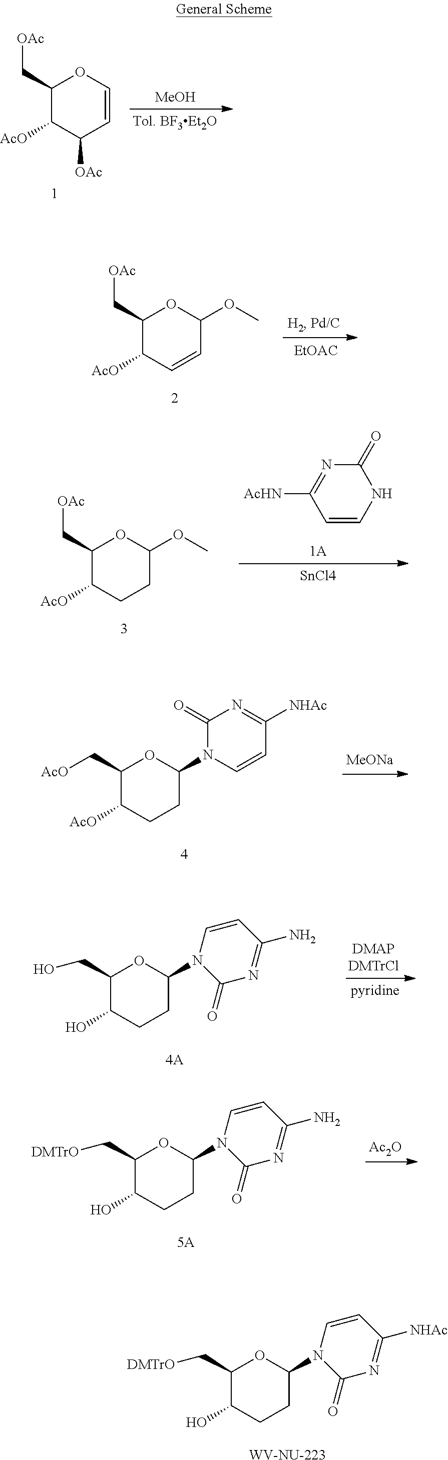

1. Preparation of Compound 2

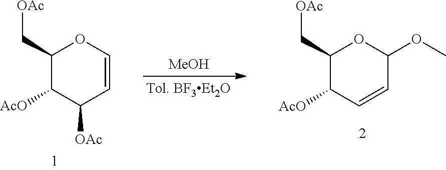 [SMILES: "MeOH" ; CC(=O)OC[C@H]1OC=C[C@@H](OC(C)=O)[C@@H]1OC(C)=O ; COC1C=C[C@H](OC(C)=O)[C@@H](COC(C)=O)O1]

BORON TRIFLUORIDE DIETHYL ETHERATE (22.16 g, 156.11 mmol) was added to a solution of Compound 1 (50 g, 183.65 mmol) in MeOH (12.95 g, 404.04 mmol, 16.35 mL, 2.2 eq.) and Tol. (600 mL) at 0° C. The mixture was stirred at 20° C. for 6.5 hrs. TLC indicated compound 1 was consumed completely and two new spots formed. The reaction was clean according to TLC. The solution was cooled to 0° C., and quenched with Et₃N (20.45 mL), after maintaining for 10 min at 0° C., Na₂CO₃ (19.45 g) was added to the solution. The filtrate was concentrated to give a residue, which was purified by silica gel column chromatography (SiO₂, Petroleum ether/Ethyl acetate=100/1 to 1/1, with 1.0 vol % Et₃N). Compound 2 (44 g, 98.09% yield) was obtained as a colorless oil.

¹H NMR (CHLOROFORM-d, 400 MHz): δ=5.73-5.87 (m, 2H), 5.25 (dd, J=9.7, 1.5 Hz, 1H), 4.86 (s, 1H), 4.13-4.25 (m, 2H), 3.93-4.03 (m, 1H), 3.39 (s, 3H), 2.04 (s, 3H), 2.02 ppm (s, 4H)

TLC (Petroleum ether:Ethyl acetate=5:1) Rf=0.23

### 2. Preparation of Compound 3

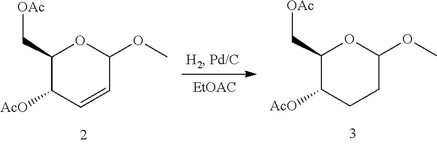 [SMILES: COC1C=C[C@H](OC(C)=O)[C@@H](COC(C)=O)O1 ; COC1CC[C@H](OC(C)=O)[C@@H](COC(C)=O)O1]

To a solution of compound 2 (44 g, 180.15 mmol) in EtOAc (400 mL) was added Pd/C (0.07 mg, 180.15 mmol, 10% purity). The mixture was stirred at 25° C. for 18 hrs under H₂ (15 Psi) atmosphere. TLC indicated compound 2 was consumed completely and one new spot formed. The reaction was clean according to TLC. The insoluble material was removed by filtration through a pad of celite. The pad was washed with EtOAc (100 mL). The filtrate was concentrated. The residue was purified by column chromatography (SiO₂, Petroleum ether/Ethyl acetate=100/1 to 0/1). Compound 3 (43 g, 96.93% yield) was obtained as a colorless oil.

¹H NMR (CHLOROFORM-d, 400 MHz): δ=4.67-4.80 (m, 2H), 4.22-4.31 (m, 1H), 4.11 (dd, J=11.9, 2.3 Hz, 1H), 3.90 (ddd, J=10.0, 5.3, 2.3 Hz, 1H), 3.37 (s, 3H), 2.09 (s, 3H), 2.04-2.06 (m, 3H), 1.81-2.02 ppm (m, 4H)

TLC: (Petroleum ether:Ethyl acetate=5:1) R_{f}=0.30

### 3. Preparation of Compound 4

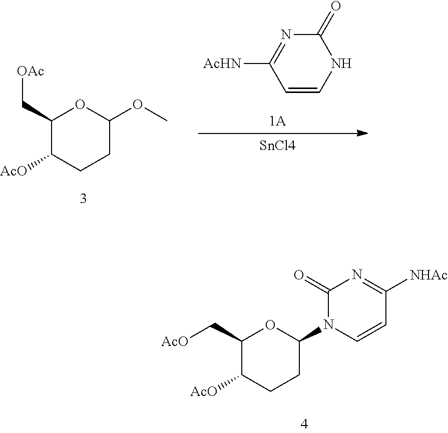 [SMILES: COC1CC[C@H](OC(C)=O)[C@@H](COC(C)=O)O1 ; "SnCl4" ; CC(=O)Nc1cc[nH]c(=O)n1 ; CC(=O)Nc1ccn([C@H]2CC[C@H](OC(C)=O)[C@@H](COC(C)=O)O2)c(=O)n1]

To a solution of compound 1A (37.44 g, 244.46 mmol) in MeCN (500 mL) was added BSA (71.04 g, 349.23 mmol, 86.32 mL) and compound 3 (43 g, 174.61 mmol) and SnCl₄ (68.24 g, 261.92 mmol, 30.60 mL). The mixture was stirred at 45° C. for 2 hr. TLC indicated compound 1A was consumed completely and two new spot formed. The reaction was clean according to TLC. The reaction mixture was partitioned between H₂O (200 mL) and EtOAc (500 mL). The organic phase was separated, washed with brine 30 mL, dried over Na₂SO₄, filtered and concentrated under reduced pressure to give a residue. The residue was purified by column chromatography (SiO₂, Petroleum ether/Ethyl acetate=100/1 to 0/1). Compound 4 (34 g, crude) was obtained as a yellow oil.

¹H NMR (DMSO-d₆, 400 MHz): δ=9.77 (br s, 1H), 8.70 (br s, 1H), 8.05 (d, J=7.9 Hz, 1H), 7.31 (br s, 1H), 6.68 (br s, 1H), 6.11-6.21 (m, 1H), 5.71-5.83 (m, 1H), 4.67 (td, J=10.2, 4.6 Hz, 1H), 4.03-4.14 (m, 2H), 2.14 (dt, J=7.8, 4.0 Hz, 1H), 2.03 (d, J=7.4 Hz, 6H), 1.81-1.95 ppm (m, 3H)

TLC (Ethyl acetate:Dichloromethane=10:1) Rf=0.46

### 4. Preparation of Compound 4A

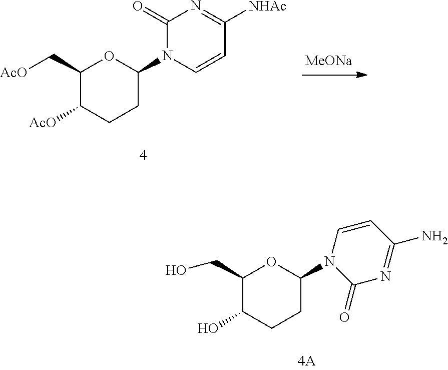 [SMILES: "MeONa" ; CC(=O)Nc1ccn([C@H]2CC[C@H](OC(C)=O)[C@@H](COC(C)=O)O2)c(=O)n1 ; Nc1ccn([C@H]2CC[C@H](O)[C@@H](CO)O2)c(=O)n1]

To a solution of compound 4 (24 g, 65.33 mmol) in MeOH (5 mL) was added CH₃ONa (10.59 g, 196.00 mmol). The mixture was stirred at 15° C. for 12 hr. HPLC showed compound 4 was consumed completely and one main peak with desired mass was detected. The filter liquor was concentrated in vacuo. The crude product was purified by reverse-phase HPLC (0.1% FA condition). Compound 4A (13 g, 82.48% yield) was obtained as a white solid.

### 5. Preparation of Compound 5A

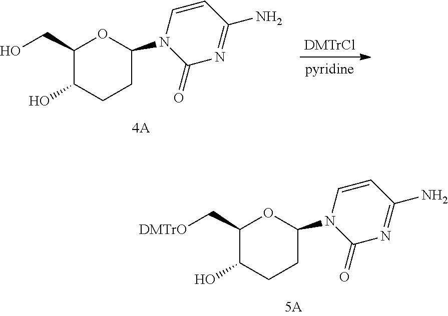

To a solution of compound 4A (13 g, 53.89 mmol) in pyridine (200 mL) was added DMT-Cl (18.26 g, 53.89 mmol). The mixture was stirred at 25° C. for 6 hr. LCMS showed compound 4A was consumed completely and one main peak with desired m/z. The reaction mixture was partitioned between H₂O (500 mL) and EtOAc (1000 mL). The organic phase was separated, washed with brine (50 mL), dried over Na₂SO₄, filtered and concentrated under reduced pressure to give a residue. The residue was purified by column chromatography (SiO₂, Dichloromethane: Methanol=100/1 to 0/1). Compound 5A (13 g, 44.38% yield) was obtained as a white solid.

LCMS: (M−H⁺)=542

TLC (Dichloromethane:Methanol=10:1), R_{f}=0.33

### 6. Preparation of WV-NU-223

(N-(1-((2R,5S,6R)-6-((bis(4-methoxyphenyl)(phenyl)methoxy)methyl)-5-hydroxytetrahydro-2H-pyran-2-yl)-2-oxo-1,2-dihydropyrimidin-4-yl)acetamide).

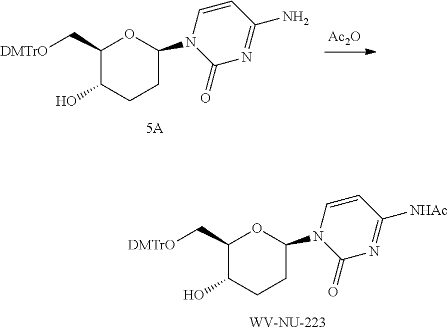

To a solution of compound 5A (13 g, 23.91 mmol) in Pyr. (130 mL) was added Ac₂O (2.44 g, 23.91 mmol, 2.24 mL). The mixture was stirred at 25° C. for 12 hr. TLC (Petroleum ether:Ethyl acetate=0:1) indicated compound 5A was consumed completely and one new spot formed. The reaction was clean according to TLC. The mixture was concentrated in vacuum. The residue was purified by column chromatography (SiO₂, Petroleum ether/Ethyl acetate=100/1 to 0/1). Compound WV-NU-223 (7.7 g, 52.78% yield, 96% purity) was obtained as a white solid.

¹H NMR (DMSO-d₆, 400 MHz): δ=10.93 (s, 1H), 8.12 (d, J=7.5 Hz, 1H), 7.40 (br d, J=7.4 Hz, 2H), 7.14-7.34 (m, 8H), 6.79-6.91 (m, 4H), 5.73 (br d, J=10.4 Hz, 1H), 4.89 (d, J=6.1 Hz, 1H), 3.73 (s, 6H), 3.59-3.66 (m, 1H), 3.26 (br d, J=9.4 Hz, 1H), 3.15 (br dd, J=10.0, 6.7 Hz, 1H), 2.12 (s, 3H), 1.93 (br d, J=10.2 Hz, 2H), 1.53-1.72 ppm (m, 2H)

LCMS=(M−H⁺)=585.6

TLC (Petroleum ether:Ethyl acetate=0:1), R_{f}=0.43

### Synthesis of WV-NU-286

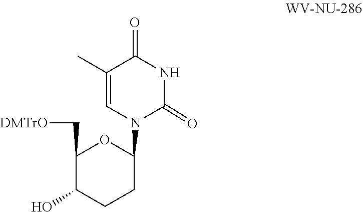

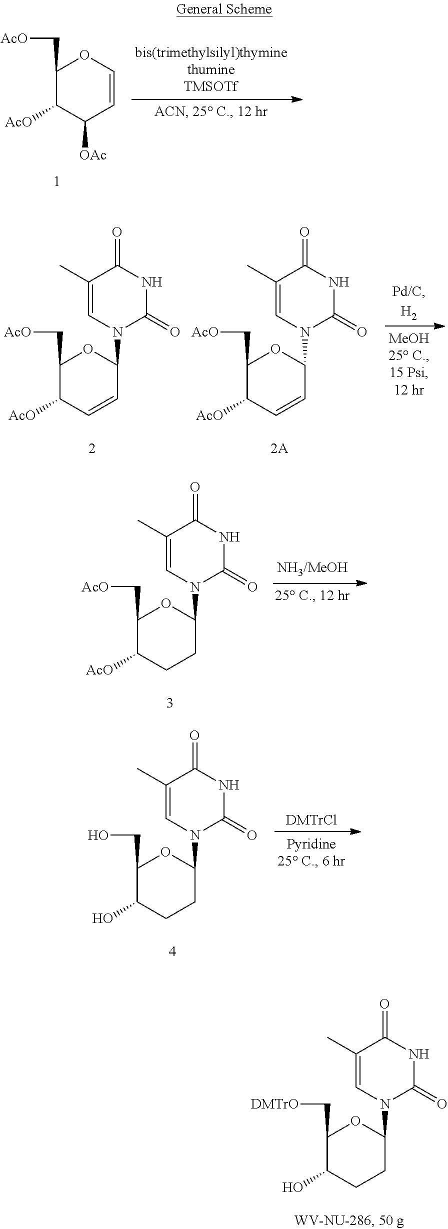

### 1. Preparation of Compound 2

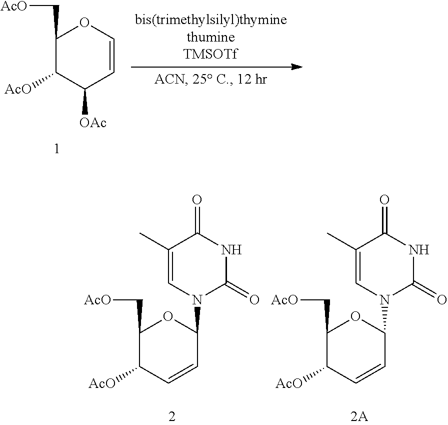

For 4 batches: A mixture of compound 1 (50 g, 183.65 mmol), 5-methyl-2-trimethylsilyloxy-2,3-dihydro-1H-pyrimidin-4-one (44.15 g, 220.39 mmol), TMSOTf (40.00 g, 179.98 mmol) in ACN (200 mL) was degassed and purged with N₂ for 3 times, and then the mixture was stirred at 25° C. for 5 hr under N₂ atmosphere. TLC indicated compound 1 was consumed completely and two new spots formed. The reaction mixture was combined and concentrated under reduced pressure to remove ACN. The residue was diluted with H₂O 2000 mL and extracted with EtOAc (1000 mL*2). The combined organic layers were washed with NaCl 500 mL, dried over Na₂SO₄, filtered and concentrated under reduced pressure to give a residue. The residue was purified by column chromatography (SiO₂, Petroleum ether/Ethyl acetate=1/0 to 0/1) to get compound 1 (120 g, 50.00% yield) was obtained as a yellow oil.

TLC (Petroleum ether:Ethyl acetate=0:1), Rf1=0.57, Rf2=0.49

### 2. Preparation of Compound 3

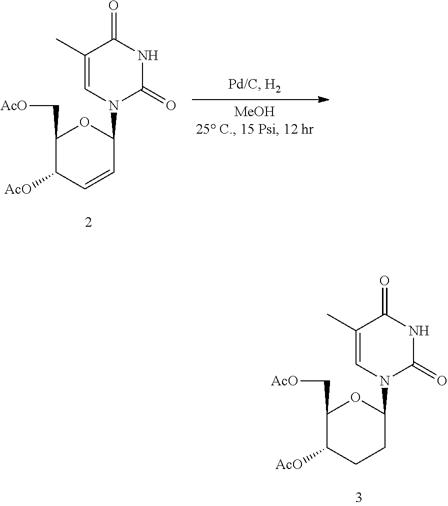 [SMILES: CC(=O)OC[C@H]1O[C@@H](n2cc(C)c(=O)[nH]c2=O)C=C[C@@H]1OC(C)=O ; CC(=O)OC[C@H]1O[C@@H](n2cc(C)c(=O)[nH]c2=O)CC[C@@H]1OC(C)=O]

For 3 batches: A mixture of compound 2 (27 g, 79.81 mmol), Pd/C (5 g, 10% purity) in EtOAc (300 mL) was degassed and purged with H₂ for 3 times, and then the mixture was stirred at 20° C. for 10 hr under H₂ atmosphere. LCMS showed compound 2 was consumed completely and desired mass was detected. The reaction mixture was combined and filtered, the filtrate concentrated under reduced pressure to give a residue to get compound 3 (78 g, crude) was obtained as a colorless oil.

¹HNMR (400 MHz, DMSO-d₆) δ=11.36 (s, 1H), 7.65 (d, J=1.1 Hz, 1H), 5.73 (dd, J=1.9, 10.8 Hz, 1H), 4.67 (dt, J=4.6, 10.3 Hz, 1H), 4.08 (d, J=4.0 Hz, 2H), 3.91 (td, J=4.0, 9.8 Hz, 1H), 2.16-2.05 (m, 2H), 2.03 (s, 3H), 2.01 (s, 3H), 1.84-1.72 (m, 5H)

LCMS (M+H+): 341.2

### 3. Preparation of Compound 4

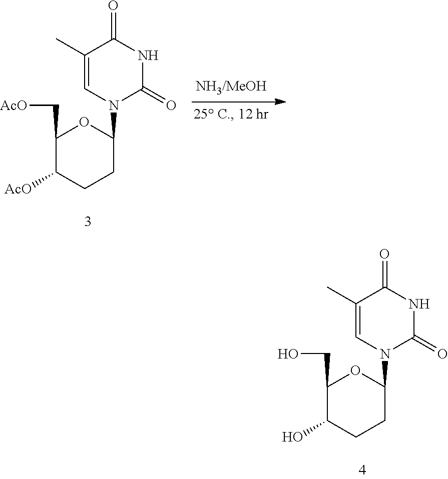 [SMILES: CC(=O)OC[C@H]1O[C@@H](n2cc(C)c(=O)[nH]c2=O)CC[C@@H]1OC(C)=O ; Cc1cn([C@H]2CC[C@H](O)[C@@H](CO)O2)c(=O)[nH]c1=O]

For three batches: To a solution of compound 3 (26 g, 76.40 mmol, 1 eq) was added NH₃/MeOH (7 M, 500 mL). The mixture was stirred at 25° C. for 10 hr. LCMS showed compound 3 was consumed completely and one main peak with desired mass was detected. The reaction mixture was concentrated under reduced pressure to get the crude. The residue was purified by column chromatography (SiO₂, DCM/MeOH=20/0 to 10/1). Compound 4 (15 g, 94.38% yield) was obtained as a white solid.

LCMS (M+H⁺): 257.2

TLC (DCM/MeOH=10:1), Rf=0.3

### 4. Preparation of Compound WV-NU-286

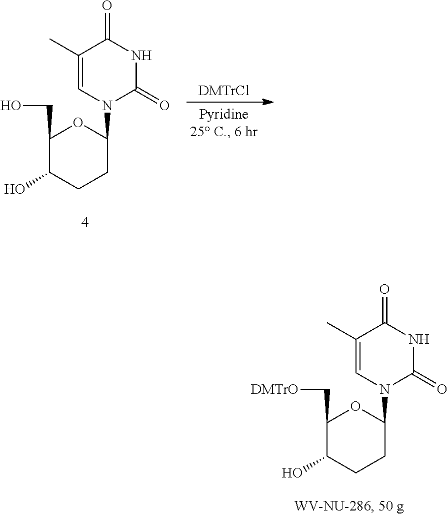

For 2 batches: To a solution of compound 4 (27 g, 105.36 mmol) in Py (700 mL) was added DMTCl (42.84 g, 126.44 mmol). The mixture was stirred at 25° C. for 6 hr. LCMS showed compound 4 was consumed completely and one main peak with desired mass was detected. The reaction mixture was concentrated under reduced pressure to remove pyridine. The residue was purified by column chromatography (SiO₂, Petroleum ether/Ethyl acetate=1/0 to 0/1, 5% TEA). WV-NU-286 (62.64 g, 38.5% yield) was obtained as a yellow solid.

¹HNMR (400 MHz, DMSO-d₆) δ=11.42-11.38 (m, 1H), 7.60 (s, 1H), 7.42-7.38 (m, 2H), 7.31-7.16 (m, 8H), 6.86-6.78 (m, 4H), 5.67-5.60 (m, 1H), 4.84 (d, J=6.0 Hz, 1H), 3.73-3.70 (m, 6H), 3.61-3.54 (m, 1H), 3.31-3.20 (m, 2H), 3.09 (br dd, J=6.7, 9.9 Hz, 1H), 1.89-1.80 (m, 4H), 1.76 (br d, J=11.3 Hz, 1H), 1.62-1.53 (m, 1H)

LCMS: (M−H+): 557.2, LCMS purity: 97.48% purity

TLC (Petroleum ether:Ethyl acetate=1:1), Rf=0.3

### Synthesis of WV-NU-287

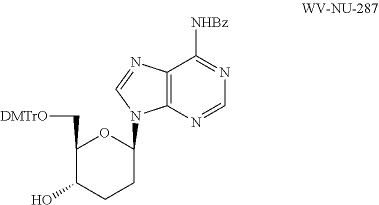

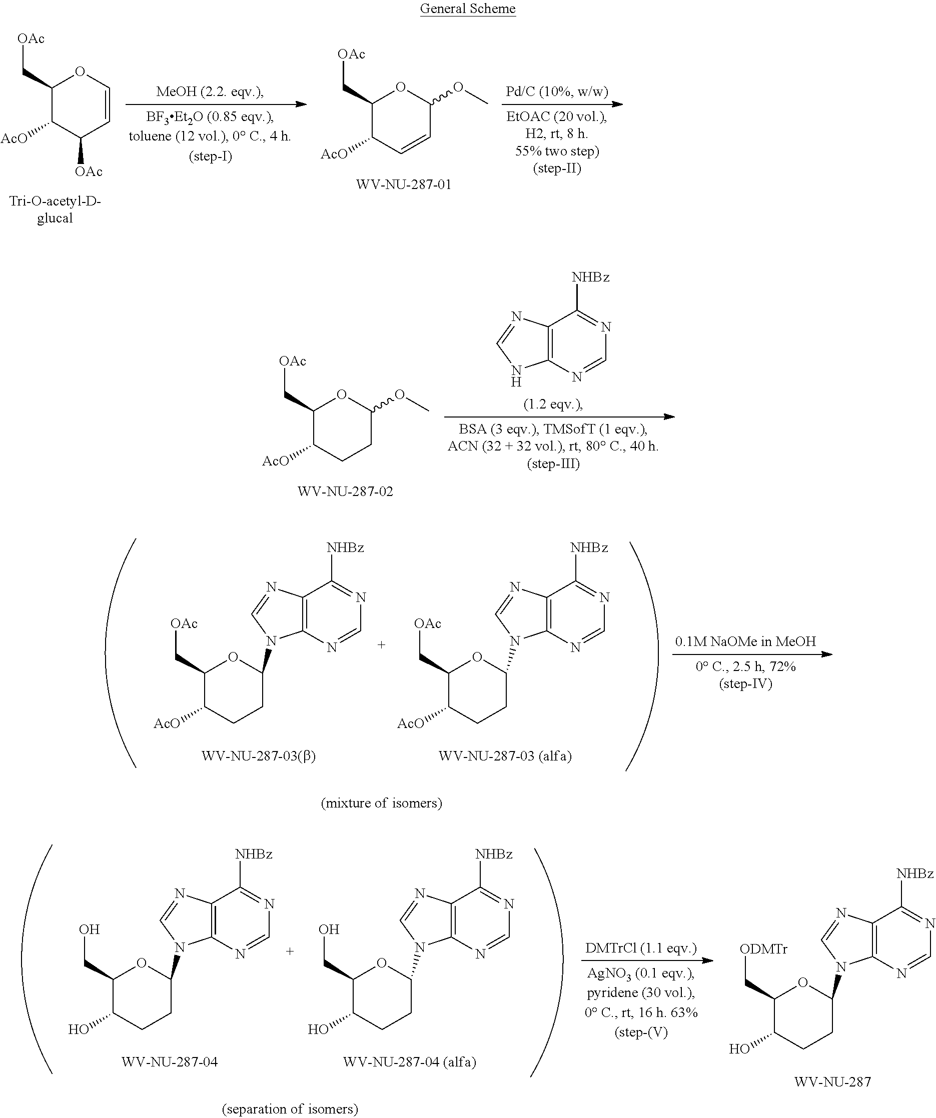

### 1. Preparation of ((2R,3S)-3-acetoxy-6-(6-benzamido-9H-purin-9-yl)tetrahydro-2H-pyran-2-yl)methyl acetate (WV-NU-287-03)

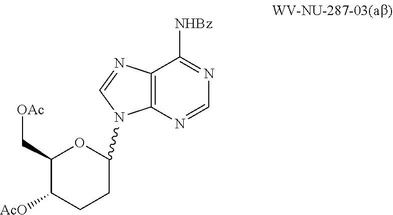

To a stirred solution of N-(9H-purin-6-yl)benzamide (100 g, 0.416 mol)) in dry acetonitrile (3.2 L, 32 vol.) was added BSA (305 mL, 1.25 mol) dropwise over a period of 20 min. The resulting mixture was heated to 80° C. and kept for 3 h. Then the mass was allowed to rt and concentrated under vacuum to get a thick mass. The mass was again dissolved in dry acetonitrile (3.2 L) and (WV-NU-287-02) (102.5 g, 0.416 mol) was added followed by TMSOTf (75.8 mL, 0.416 mol) dropwise over a period of 40 min. The reaction mixture was stirred at 80° C. for 40 h. Progress of the reaction was monitored by TLC. The reaction mixture was concentrated under reduced pressure. The crude mass was dissolved in EtOAC (1 L), washed with sat. NaHCO₃ (250 mL×2), brine (250 mL×1), dried over Na₂SO₄ and concentrated under vacuum to afford as a light yellow solid. The solid was purified by column chromatography over silica gel (230-400 mesh) eluted in 3% MeOH/DCM to get a yellowish solid. (WV-NU-287-03) (57 g, 30% (isomeric mixture of α and β), TLC Mobile phase details: 7% MeOH in DCM. ¹H NMR (400 MHz, DMSO-d₆): δ in ppm=11.23 (s, 1H), 8.78 (d, 1H, J1=3.4 Hz), 8.74 (s, 1H), 8.05 (d, 2H, J1=7.3 Hz), 7.65 (m, 1H), 7.56 (m, 3H), 6.07 (dd, 1H, J1=6.9 Hz, J2=4.0 Hz), 4.81 (m, 1H), 4.07 (m, 3H), 2.64 (m, 1H), 2.21 (m, 2H), 2.07 (d, 3H, J1=3 Hz), 1.94 (m, 4H). MS: m z calcd for C₂₂H₂₃N₅O₆, 453.2; found 454.48. [M+H⁺].

### 2. Preparation of N-(9-((2R,5S,6R)-5-hydroxy-6-(hydroxymethyl)tetrahydro-2H-pyran-2-yl)-9H-purin-6-yl)benzamide (WV-NU-287-04)

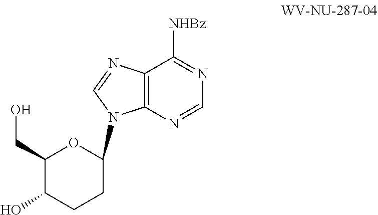

Compound (WV-NU-287-03) (57 g, 0.1213 mol) was treated with a solution of (0.1 M) NaOMe in methanol (1.7 L, 30 vol.) at 0° C. and maintained for 2.5 h. Progress of the reaction was monitored by TLC. The reaction mixture was neutralized with acetic acid (PH=7.0) and mixture was concentrated under vacuum to get a solid. The solid mass was purified by column chromatography over silica gel (230-400 mesh) eluted in 8% MeOH/DCM as a white solid (45 g) (mixture of a and R isomer). The solid was dissolved in (methanol: water) (1:1) (10 vol.) and stirred at 60° C. for 20 min, a clear solution was observed, kept at rt for 20 h. A solid precipitate was observed which was filtered off and dried under vacuum to get an off white solid. (WV-NU-287-04) (16 g, almost 100% β-isomer). TLC Mobile phase details: 10% MeOH in DCM. ¹H NMR (500 MHz, DMSO-d₆): δ in ppm=11.2 (s, 1H), 8.77 (s, 1H), 8.71 (s, 1H), 8.05 (d, 2H, J1=7.3 Hz), 7.65 (m, 1H), 7.55 (m, 2H), 5.87 (m, 1H), 4.98 (d, 1H, J1=5.1 Hz), 4.58 (t, 1H, J1=6.0 Hz), 3.70 (dd, 1H, J1=10.9 Hz, J2=6.6 Hz), 3.46 (m, 3H), 2.43 (m, 1H), 2.12 (m, 2H), 1.69 (m, 1H). MS: m czalcd for C₁₈H₁₉N₅O₄, 369.4.

### 3. Preparation of N-(9-((2R,5S,6R)-6-((bis(4-methoxyphenyl)(phenyl)methoxy)methyl)-5-hydroxy tetrahydro-2H-pyran-2-yl)-9H-purin-6-yl)benzamide (WV-NU-287)

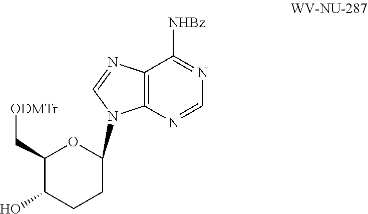

To a stirred solution of (WV-NU-287-04) (25 g, 0.06776 mol) in anhydrous Pyridine (750 mL, 30 vol.) was added Silver nitrate (1.13 g, 0.00677), DMTCl (25.18 g, 0.0745 mol) portion-wise over a period of 30 min at 0° C. Above reaction was stirred at for 16 h. Progress of the reaction was monitored by TLC. Then reaction was concentrated under vacuum to get crude mass. The crude dissolved in ethyl acetate (250 mL), washed with sat. NaHCO₃ (60 mL×2), brine solution (60 mL×1), dried over Na₂SO₄, concentrated and purified by column chromatography over silica gel (230-400 mesh) eluted in 3% EtOH/DCM to get as an off white solid (WV-NU-287) (29 g, 63%, almost 100% 0-isomer).. TLC Mobile phase details: 10% EtOH in DCM. ¹H NMR (400 MHz, DMSO-d₆): δ in ppm=11.24 (s, 1H), 8.81 (s, 1H), 8.71 (s, 1H), 8.05 (m, 2H), 7.65 (m, 1H), 7.55 (m, 2H), 7.36 (m, 2H), 7.19 (m, 7H), 6.75 (m, 4H), 5.96 (d, 1H, J1=9.8 Hz), 4.94 (d, 1H, J1=5.9 Hz), 3.73 (m, 1H), 3.69 (d, 6H, J1=8.7 Hz), 3.43 (m, 2H), 3.26 (d, 1H, J1=8.7 Hz), 3.05 (dd, 1H, J1=10.01 Hz, J2=6.8 Hz), 2.15 (d, 2H, J1=11.1 Hz), 1.71 (m, 1H), 1.19 (m, 1H), 1.06 (t, 1H, J1=6.9 Hz), MS: m czalcd for C₃₉H₃₇N₅O₆, 671.8; found 672.8 (M+H⁺).

### Synthesis of WV-NU-288

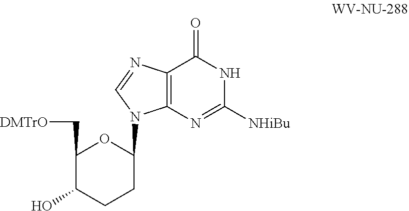

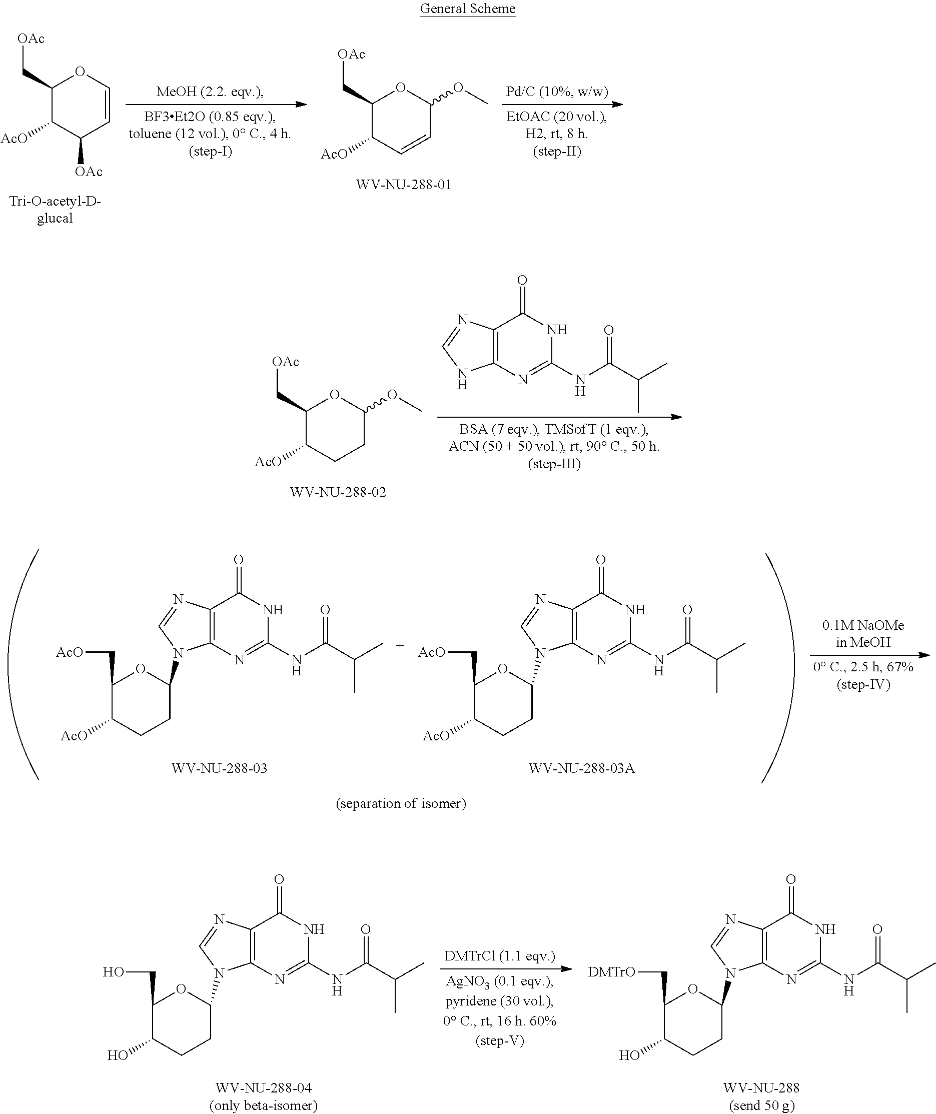

### 1. Preparation of ((2R,3S)-3-acetoxy-6-methoxy-3,6-dihydro-2H-pyran-2-yl)methylacetate (WV-NU-288-01)

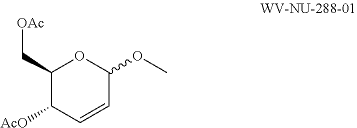 [SMILES: COC1C=C[C@H](OC(C)=O)[C@@H](COC(C)=O)O1]

To a stirred solution of tri-O-acetyl-D-glucal (300 g, 1.102 mol) and dry methanol (32.6 mL, 2.426 mol.) in dry toluene (3 L, 10 vol.) was added boron trifluoride-ether complex (108 mL, 0.882 mol) dropwise over a period of 50 mins with vigorous stirring at 0° C. The reaction was maintained for 5 h at 0° C. Progress of the reaction was monitored by TLC. Then reaction mixture was quenched with trimethylamine (120 mL) at 0° C. and maintained for 20 min. After that sodium carbonate (106 g) was added to the solution. Then the mass was filtered and filtrate was washed washed with EtOAC (200 mL×3) dried over Na₂SO₄and concentrated under vacuum to get gummy syrup (WV-NU-288-01) (320 g, crude). TLC Mobile phase details: 20% EtOAC in Hexane. ¹H NMR (400 MHz, CDCl₃): δ in ppm=5.92 (m, 2H), 5.32 (m, 1H, J1=9.7 Hz, J2=1.5 Hz), 4.93 (d, 1H, J1=0.7 Hz), 4.23 (m, 2H), 4.08 (m, 2H), 3.47 (d, 3H, J1=5.8 Hz), 2.10 (s, 6H).

### 2. Preparation of ((2R,3S)-3-acetoxy-6-methoxytetrahydro-2H-pyran-2-yl)methyl acetate (WV-NU-288-02)

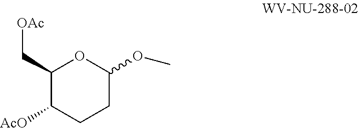 [SMILES: COC1CC[C@H](OC(C)=O)[C@@H](COC(C)=O)O1]

A solution of (WV-NU-288-01) (330 g, 1.341 mol) in dry EtOAC (3.3 L, 10 vol.) was Flushed with Ar gas and 10% Pd/C (30.3 g, 10 mol wt./wt.)) was added at rt. The system was filled up with H₂ gas and stirred at rt, for 8 h. Progress of the reaction was monitored by TLC. After that reaction mass was filtered through celite washed with EtOAC (200 mL×3) and concentrated under vacuum to get gummy mass. The mass was purified by column chromatography over neutral silica gel (230-400 mesh) eluted in 20% EtOAC/Hexane to get as a light yellowish oil. (WV-NU-288-02) (132 g, 49% for 2 step). TLC Mobile phase details: 20% EtOAC in Hexane. ¹H NMR (400 MHz, CDCl₃): δ in ppm=4.73 (m, 2H), 4.27 (ddd, 1H, J1=12.0 Hz, J2=5.3 Hz, J3=3.1 Hz), 4.15 (m, 2H), 3.91 (dq, 1H, J1=5.1 Hz, J2=2.2 Hz), 3.47 (s, 3H), 2.09 (s, 4H), 2.04 (s, 4H), 1.98 (m, 1H), 1.82 (m, 3H).

### 3. Preparation of ((2R,3S)-3-acetoxy-6-(2-isobutyramido-6-oxo-1,6-dihydro-9H-purin-9-yl)tetrahydro-2H-pyran-2-yl)methyl acetate (WV-NU-288-03)

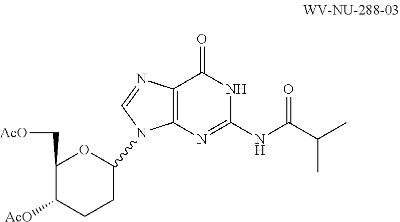 [SMILES: CC(=O)OC[C@H]1OC(n2cnc3c(=O)[nH]c(NC(=O)C(C)C)nc32)CC[C@@H]1OC(C)=O]

To a stirred solution of N-(6-oxo-6,9-dihydro-1H-purin-2-yl)isobutyramide (100 g, 0.452 mol)) in dry acetonitrile (5 L, 50 vol.), was added BSA (553 mL, 2.262 mol) dropwise over a period of 30 min. The resulting mixture was warmed to 90° C. for 24 h. Then reaction mass was allowed to cool to rt and concentrated under vacuum to get thick syrup, The mass was again dissolved in dry acetonitrile (5 L, 50 vol.) and (WV-NU-288-02) (111.2 g, 0.452 mol) was added followed by TMSOTf (83.2 mL, 0.452 mol) dropwise over a period of 40 min. Then reaction mixture was stirred at 90° C. for 50 h. Progress of the reaction was monitored by TLC. The reaction mixture concentrated under reduced pressure. The crude mass was dissolved in EtOAC (1 L), washed with sat. NaHCO₃ (250 mL×2), brine (200 mL×1), dried over Na₂SO₄ and concentrated under vacuum to afford as a yellowish solid. The solid was purified by column chromatography over silica gel (230-400 mesh) eluted in 3% MeOH/DCM to get as a light yellow solid (45 g, mixture of isomers). The solid was dissolved EtOAc (10 vol.), stirred for 6 h and filter-off, solid was washed with EtOAc (30 ml×2) to get pale yellow solid (WV-NU-288-03) (18.2 g, almost 100% β-isomer).), TLC Mobile phase details: 7% MeOH in DCM. ¹H NMR (400 MHz, DMSO-d₆): δ in ppm=12.14 (s, 1H), 11.72 (s, 1H), 8.25 (s, 1H), 5.68 (dd, 1H, J1=11.3 Hz, J2=2.1 Hz), 4.73 (m, 1H), 4.10 (m, 2H), 3.90 (ddd, 1H, J1=9.8 Hz, J1=5.4 Hz, J1=2.5 Hz), 2.80 (m, 1H), 2.56 (m, 1H), 2.23 (m, 1H), 2.09 (m, 1H), 2.05 (s, 3H), 1.99 (s, 3H), 1.81 (m, 1H,), 1.26 (m, 1H), 1.12 (m, 6H). MS: m z calcd for C₁₉H₂₅N₅O₇, 435.4; found 434.22. [M−H⁺].

### 4. Preparation of N-(9-((2R,5S,6R)-5-hydroxy-6-(hydroxymethyl)tetrahydro-2H-pyran-2-yl)-6-oxo-6,9-dihydro-1H-purin-2-yl)isobutyramide (WV-NU-288-04)

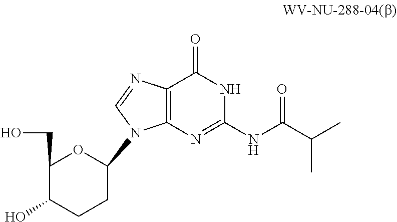 [SMILES: CC(C)C(=O)Nc1nc2c(ncn2[C@H]2CC[C@H](O)[C@@H](CO)O2)c(=O)[nH]1]

Compound (WV-NU-288-03) (50 g, 0.1149 mol) was treated with a solution of 0.1 M NaOMe in methanol (750 mL, 30 vol.) at 0° C. and maintained for 2 h. Progress of the reaction was monitored by TLC. The reaction mixture was neutralized with acetic acid (pH=7.0) and mixture was concentrated under vacuum to get solid. The solid was purified by column chromatography over silica gel (230-400 mesh) eluted in 10% MeOH/DCM to get as off white solid. (WV-NU-288-04) (28 g, 69%, almost 100% β-isomer). TLC Mobile phase details: 15% MeOH in DCM. ¹H NMR (400 MHz, DMSO-d₆): δ in ppm=12.1 (s, 1H), 8.23 (d, 1H, J1=5.6 Hz), 5.5 (dd, 1H, J1=11.1 Hz, J2=2.1 Hz), 4.84 (d, 2H, J1=13.8 Hz), 3.68 (d, 1H, J1=11.9 Hz, J2=1.9 Hz), 3.48 (dd, 1H, J1=12.0 Hz, J2=5.7 Hz), 3.40 (m, 1H), 3.30 (ddd, 1H, J1=9.2 Hz, J2=5.8 Hz, J3=2.0 Hz), 2.78 (m, 1H), 2.46 (t, 2H, J1=7.1 Hz), 2.29 (m, 1H), 2.10 (m, 1H), 2.01 (s, 1H), 1.89 (s, 1H), 1.58 (ddd, 1H, J1=24 Hz, J2=13.0 Hz, J3=3.7 Hz), 1.12 (dd, 6H, J1=6.8 Hz, J2=0.6 Hz), 0.94 (t, 3H, J1=7.1 Hz) MS: m czalcd for C₁₅H₂₁N₅O₅, 351.4; found 350.14 [M−H].

### 5. Preparation of N-(9-((2R,5S,6R)-6-((bis(4-methoxyphenyl)(phenyl)methoxy)methyl)-5-hydroxyl tetrahydro-2H-pyran-2-yl)-6-oxo-6,9-dihydro-1H-purin-2-yl)isobutyramide (WV-NU-288)

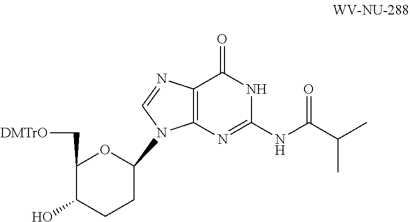

To a stirred solution of (WV-NU-287-04) (28 g, 0.0797 mol) in anhydrous Pyridine (840 mL, 30 vol.) was added Silver nitrate (1.34 g, 0.00797), DMTCl (29.65 g, 0.0877 mol) portion-wise over a period of 25 min at 0° C. Above reaction was stirred at for 18 h. Progress of the reaction was monitored by TLC. Then reaction was concentrated under vacuum to get crude mass. The crude dissolved in ethyl acetate (300 mL), washed with sat. NaHCO₃ (10 mL×2), brine solution (100 mL×1), dried over Na₂SO₄, concentrated and purified by column chromatography over basic silica gel (230-400 mesh) eluted in 4% EtOH/DCM to get as a off white solid (WV-NU-288) (30 g, 58%). TLC Mobile phase details: 10% EtOH in DCM. ¹H NMR (500 MHz, DMSO-d₆): δ in ppm=12.15 (s, 1H), 11.74 (s, 1H), 8.24 (s, 1H), 7.37 (m, 2H), 7.19 (m, 7H), 6.78 (m, 4H), 5.64 (dd, 1H, J1=11.0 Hz, J2=2.1 Hz), 4.96 (d, 1H, J1=6.2 Hz), 3.70 (d, 6H, J1=1.4 Hz), 3.61 (m, 1H), 3.38 (m, 2H), 3.25 (d, 1H, J1=9.8 Hz), 3.06 (dd, 1H, J1=10 Hz, J2=6.5 Hz), 2.81 (m, 1H), 2.47 (t, 1H, J1=2.1 Hz), 2.31 (m, 1H), 2.09 (m, 2H), 1.61 (m, 1H), 1.26 (m, 1H), 1.12 (dd, 6H, J1=6.9 Hz, J2=5.5 Hz), 0.95 (t, 1H, J1=67.2 Hz). MS: m czalcd for C₃₆H₃₉N₅O₇, 653.7; found 652.62 [M−H].

### Preparation of amidite N568-362

(N-(1-((2R,5S,6R)-6-((bis(4-methoxyphenyl)(phenyl)methoxy)methyl)-5-(((1S,3S,3aS)-3-((phenylsulfonyl)methyl)tetrahydro-1H,3H-pyrrolo[1,2-c][1,3,2]oxazaphosphol-1-yl)oxy)tetrahydro-2H-pyran-2-yl)-2-oxo-1,2-dihydropyrimidin-4-yl)acetamide).

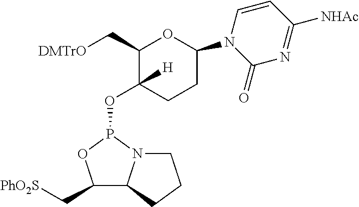

Amidite N568-362 was synthesized using general procedure from WV-NU-223. Yield, 74%. ³¹P NMR (243 MHz, CDCl₃) δ 149.44; MS (ES) m z calculated for C₄₅H₄₉N₄O₁₀PS [M+K]⁺ 907.25, Observed: 907.14 [M+K]⁺.

### Preparation of amidite N891-19

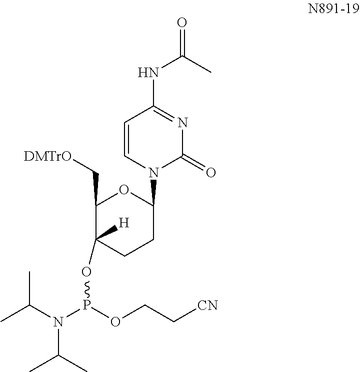

Amidite N891-19 was synthesized using general procedure from WV-NU-223. Yield, 79%. ³¹P NMR (243 MHz, CDCl₃) δ 148.37, 148.12; MS (ES) m z calculated for C₄₂H₅₂N₅O₈P [M+K]⁺824.32, Observed: 824.59 [M+K]⁺.

### Preparation of Amidite N891-6

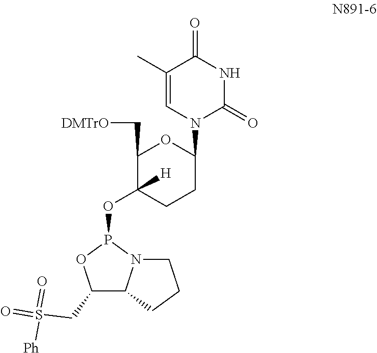

Amidite N₈₉₁-6 was synthesized using general procedure from WV-NU-286. Yield, 62%.

³¹P NMR (243 MHz, CDCl₃) δ 157.82; MS (ES) m z calculated for C₄₄H₄₈N₃O₁₀PS [M+K]+880.24, Observed: 880.32 [M+K]⁺.

### Preparation of amidite N891-7

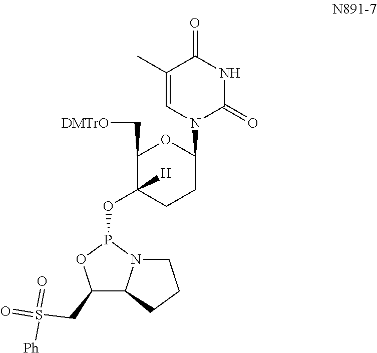

Amidite N891-7 was synthesized using general procedure from WV-NU-286. Yield, 57%. ³¹P NMR (243 MHz, CDCl₃) δ 157.82; MS (ES) m z calculated for C₄₄H₄₈N₃O₁₀PS [M+K]⁺ 880.24, Observed: 880.32 [M+K]⁺.

### Preparation of amidite N920-3

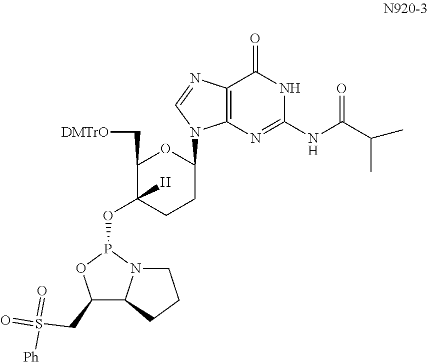

Amidite N920-3 was synthesized using general procedure from WV-NU-288. Yield, 76%. ³¹P NMR (243 MHz, CDCl₃) δ 151.21; MS (ES) m/z calculated for C₄₈H₅₃N₆O₁₀PS [M+Na]⁺959.32, Observed: 959.08 [M+Na]⁺.

### Preparation of amidite N891-13

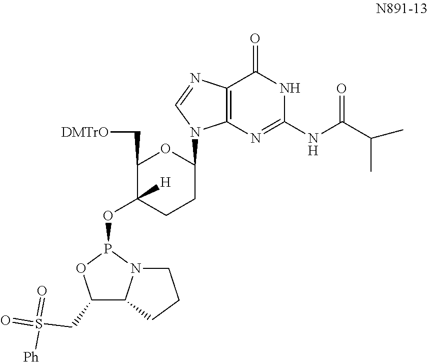

Amidite N₈₉₁-13 was synthesized using general procedure from WV-NU-288. Yield, 72%. ³¹P NMR (243 MHz, CDCl₃) δ 158.34; MS (ES) m z calculated for C₄₈H₅₃N₆O₁₀PS [M+Na]⁺ 935.33, Observed: 935.67 [M]⁻.

### Preparation of amidite N891-38

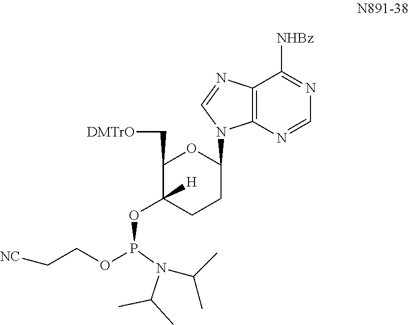

Amidite N891-38 was synthesized using general procedure from WV-NU-287. Yield, 68%. ³¹P NMR (243 MHz, CDCl₃) δ 148.47, 148.15; MS (ES) m z calculated for C₄₈H₅₄N₇O₇P [M]⁻ 871.38, Observed: 871.27 [M]⁻.

### Preparation of Amidite N920-2

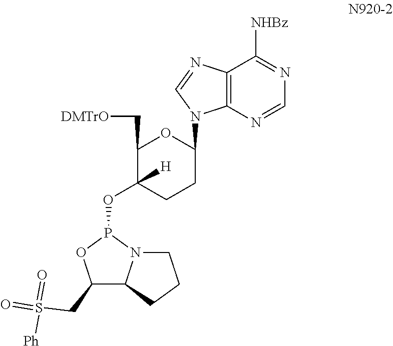

Amidite N920-2 was synthesized using general procedure from WV-NU-287. Yield, 77%.

³¹P NMR (243 MHz, CDCl₃) δ 150.71; MS (ES) m z calculated for C₅₁H₅₁N₆O₉PS [M+Na]⁺977.31, Observed: 977.56 [M+Na]⁺.

### Preparation of Amidite N₈₉₁-9

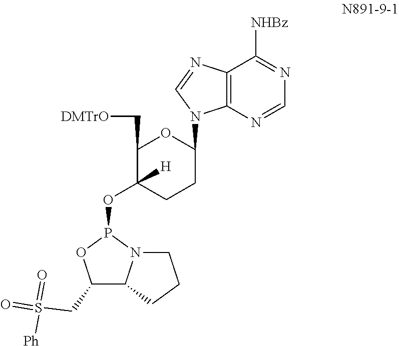

Amidite N891-9 was synthesized using general procedure from WV-NU-287. Yield, 63%. ³¹P NMR (243 MHz, CDCl₃) δ 157.7; MS (ES) m z calculated for C₅₁H₅₁N₆O₉PS [M+Na]⁺ 977.31, Observed: 977.65 [M+Na]⁺.

### Example 51: Synthesis of PN-Lipid Azides

### General Synthetic Method for Single Chain PN-Lipid Azides

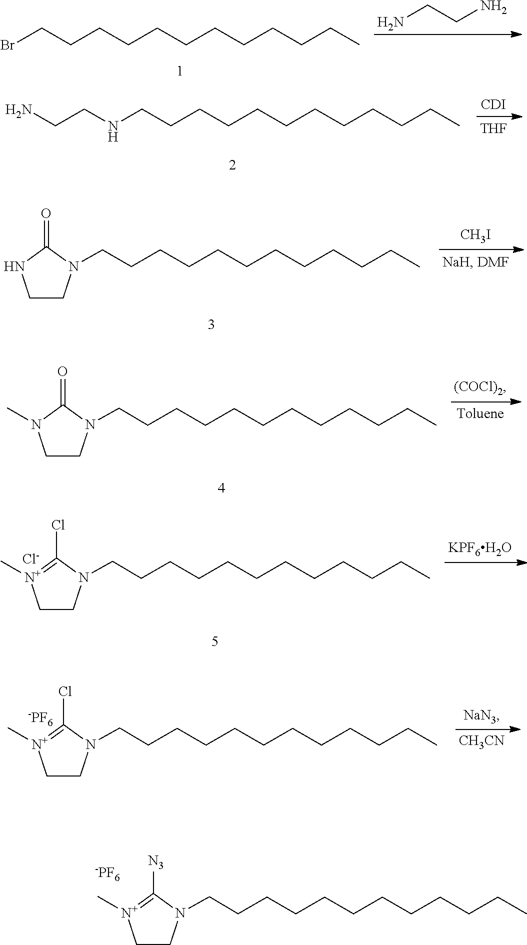

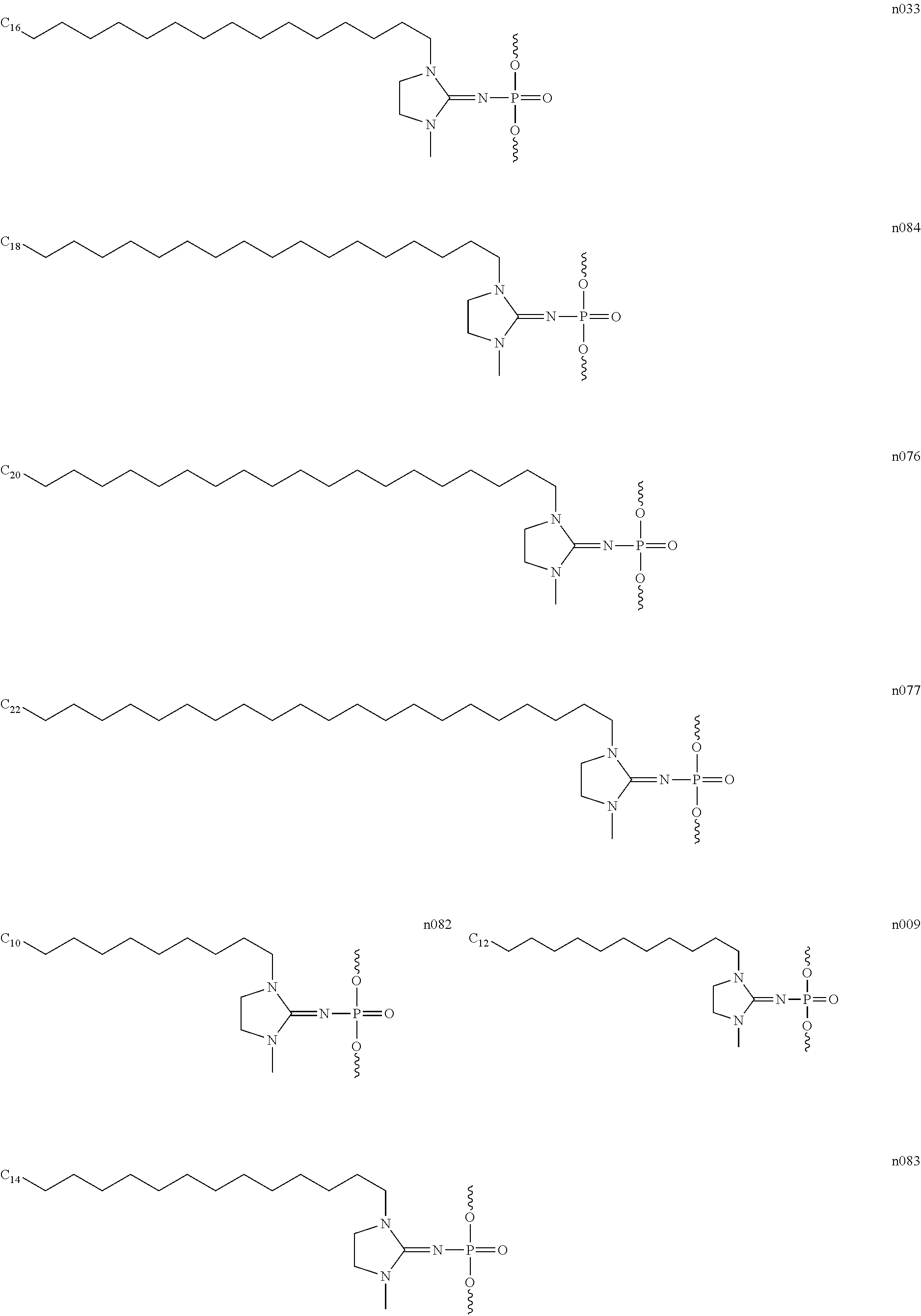

### Synthesis of WV-DL-045 (n009)

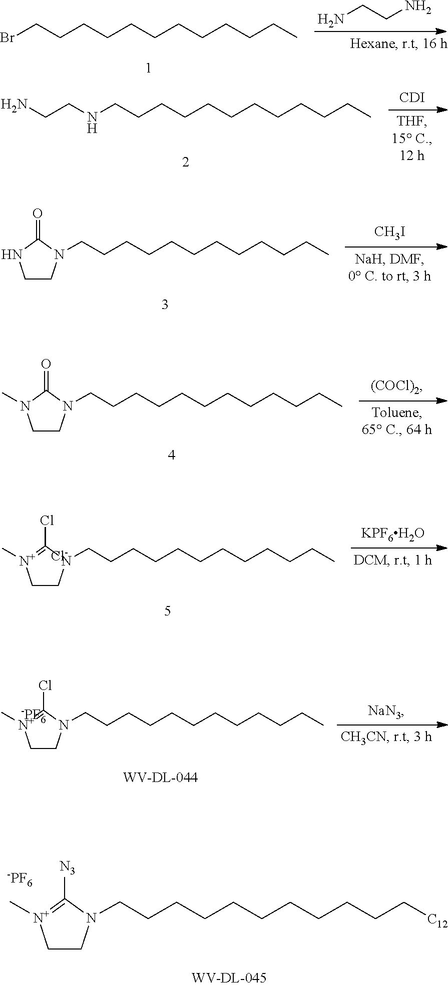

### 1. Preparation of Compound 2

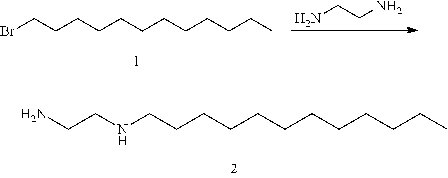

In a one-neck round bottom flask, ethane-1,2-diamine (337.59 g, 5.62 mol) was placed with a magnetic stirring bar, and compound 1 (50 g, 200.62 mmol) was added slowly at 0° C. After finishing the addition, the reaction mixture was warmed to 25° C., and left undisturbed for an additional 1 h. 300 mL of hexane was added into the reaction mixture, which was stirred vigorously for 12 h at 25° C. LCMS showed the reaction was completed, staring material was consumed and the product was obtained, the hexane layer was decanted and dried under reduced pressure to give compound 2 (123 g) crude as colorless oil. LCMS: (M+H⁺) 229.2

### 2. Preparation of Compound 3

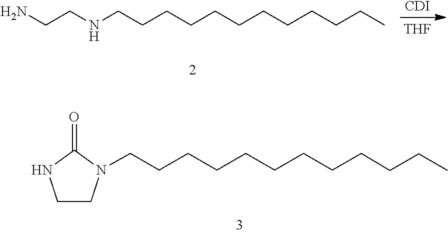

Two batches in parallel. To a solution of compound 2 (61.5 g, 269.25 mmol) and CDI (43.66 g, 269.25 mmol) in THF (630 mL) was stirred at 15° C. for 12 hr. TLC showed the reaction was completed, starting material was consumed and the product was obtained. The crude reaction mixture (126 g scale) was combined to another two batch crude product (123 g scale) and (84 g scale) for further purification. The combined crude product was purified by column chromatography on a silica gel eluted with petroleum ether: ethyl acetate (from 10/1 to 1/12) to give product 3 (95 g, 65.09% yield) as a white solid.

TLC (Ethyl acetate:Methanol=10:1) R_{f1}=0.50

### 3. Preparation of Compound 4

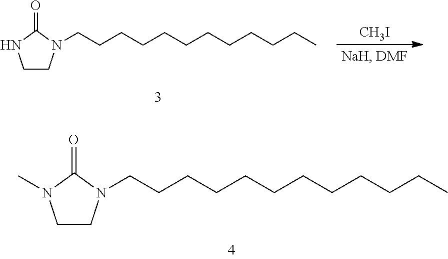

Six batches in parallel. To a solution of compound 3 (40 g, 157.23 mmol) in DMF (650 mL) was added NaH (7.55 g, 188.67 mmol, 60% purity) at 0° C. and the reaction stirred for 0.5 h, Then added CH₃I (66.95 g, 471.68 mmol) to the above reaction mixture, and stirred at 25° C. for 3 h. TLC showed the reaction was completed, starting material was consumed and the product was obtained. The reaction mixture was quenched by addition H₂O (1000 mL) at 25° C., and extracted with Ethyl acetate (1000 mL*3). The combined organic layers were dried over anhydrous Na₂SO₄, filtered and concentrated under reduced pressure to give a residue. The residue was purified by column chromatography (SiO₂, Petroleum ether/Ethyl acetate=20/1 to 1/2) to give product 4 (232 g, crude) as yellow oil.

¹H NMR (400 MHz, CHLOROFORM-d) 6=3.25-3.17 (m, 4H), 3.09 (t, J=7.3 Hz, 2H), 2.70 (d, J=1.6 Hz, 3H), 1.45-1.36 (m, 2H), 1.28-1.14 (m, 19H), 0.85-0.76 (m, 3H)

TLC (Petroleum ether:Ethyl acetate=0:1) R_{f1}=0.5

### 4. Preparation of Compound 5

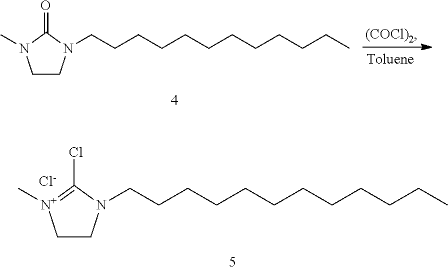

A mixture of compound 4 (30 g, 111.76 mmol, 1 eq.) in Tol.(250 mL) was degassed and purged with N₂ for 3 times, and then to the mixture was added oxalyl chloride (212.78 g, 1.68 mol, 146.75 mL, 15 eq.) and stirred at 65° C. for 72 hr under N₂ atmosphere. LCMS showed the reaction was completed, staring material was consumed, the desired product was obtained. Then the mixture was concentrated in vacuo. The white solid was washed by cooled EtOAc (100 mL*2), and then the solid was concentrated in vacuo, to give product 5 (20 g, crude) as a white solid.

LCMS: M⁺, 287.3

### 5. Preparation of Compound WV-DL-044

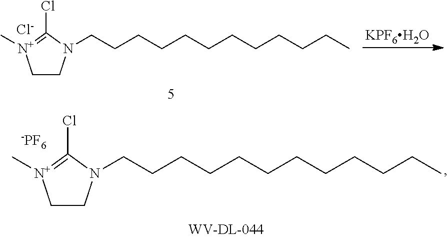

To a solution of compound 5 (8 g, 24.74 mmol) in DCM (46 mL) and H₂O (26 mL) was added potassium hexafluorophosphate (4.55 g, 24.74 mmol) at 25° C. The reaction mixture was stirred at 25° C. for 1 h. TLC showed the reaction was completed, starting material was consumed, and the desired product was obtained. The filtrate was washed with H₂O (10 mL*2), and the white solid was desired compound. To give product WV-DL-044 (6.5 g, 60.69% yield, F6P) as a white solid. The product was combined with another two batches product (2.5 g), and (2.55 g) for analysis and delivery. Finally, 11.5 g of product was got

TLC (Petroleum ether:Ethyl acetate=0:1) R_{f}=0.0

### 6. Preparation of Lipid Azide WV-DL-045

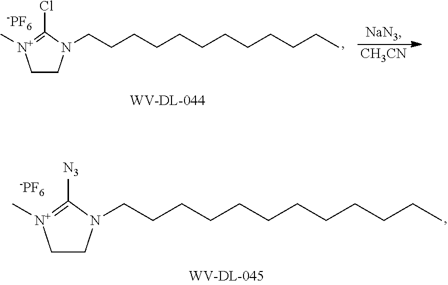

2.2 g WV-DL-044 and 495 mg NaN₃ were added to a round bottom flask. Dry ACN was added forming a suspension and stirred 2.5 hr at room temperature. The reaction mixture was filtered through a pad of celite and washed with CAN. The filtrate was dried on rotovap and was then redissolved in a minimal amount ACN and the solution was precipitated with diethyl ether to afford 1.75 g of fluffy white solid

¹H NMR (600 MHz, Chloroform-d) δ 3.87 (dd, J=12.1, 8.1 Hz, 1H), 3.81-3.75 (m, 1H), 3.29 (t, J=7.8 Hz, 1H), 3.12 (s, 2H), 1.57-1.50 (m, 1H), 1.22 (s, 3H), 1.19 (s, 6H), 0.84-0.78 (m, 2H).

¹³C NMR (151 MHz, CDCl₃) δ 154.76, 77.29, 77.07, 76.86, 49.38, 47.03, 46.52, 33.13, 31.90, 29.61, 29.61, 29.54, 29.42, 29.34, 29.05, 26.97, 26.47, 22.68, 14.11.

### Synthesis of Azide (SOPL-WLS-41, n033)

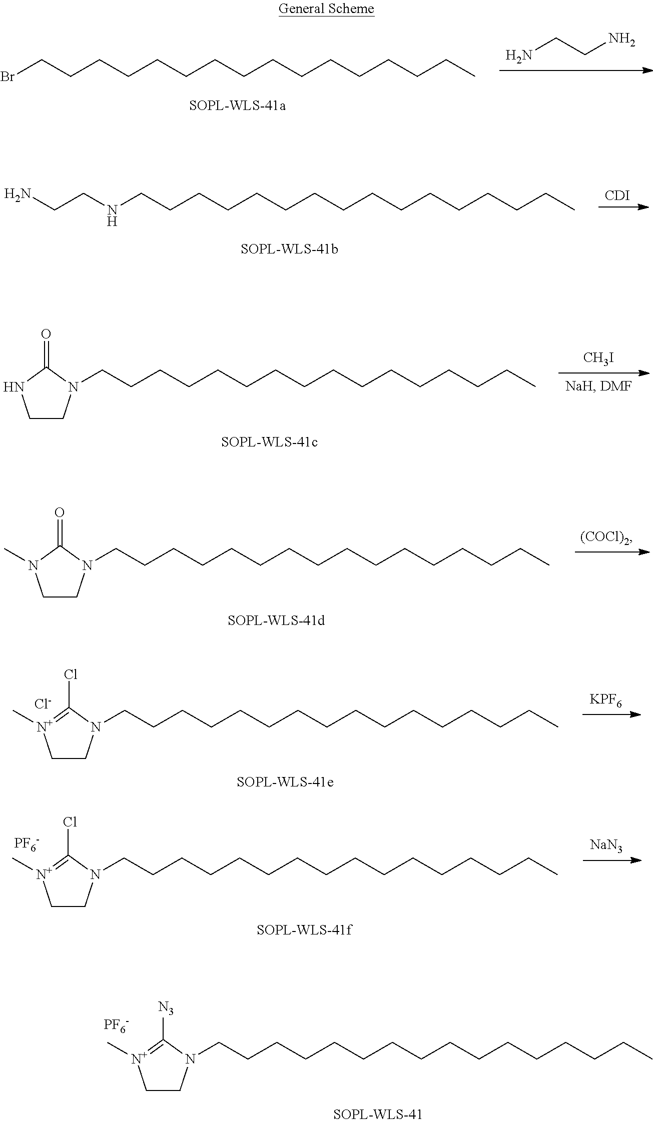

### 1. Preparation of SOPL-WLS-41b

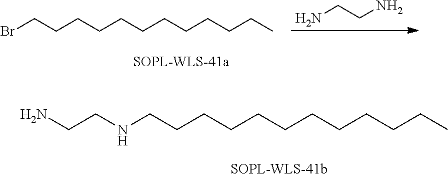

In a clean and dry two-neck 1 Lit round bottom flask, ethane-1,2-diamine (306 mL, 4.585 mol) was placed with a magnetic stirring bar, and compound SOPL-WLS-41a (50 g, 0.164 mol) was added dropwise at 0° C. by using addition funnel. After finishing the addition, the reaction mixture was warmed to 25° C., and left undisturbed for an additional 1 h. Then, 300 mL of hexane was added into the reaction mixture and stirred vigorously for 16 h at 25° C. TLC showed the reaction was completed, staring material was consumed and the new spot was formed (TLC—10% MeOH:EtOAc; TLC charring—Phosphomolybdic acid). The hexane layer was separated by using separatory funnel. Again 300 mL of hexane was added to amine layer and stir for 4 h at rt. After that hexane layer was separated and combined with previous hexane layer, dried over sodium sulphate and evaporated to dryness under reduced pressure to get compound SOPL-WLS-41b (48 g) as a crude colorless liquid.

MS: m z calcd for C₁₈H₄₀N₂ ([M+H]⁺), 285.53; found 285.38.

### 2. Preparation of SOPL-WLS-41c

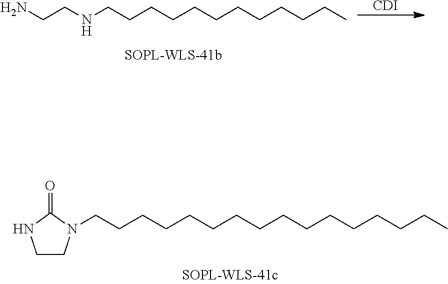

SOPL-WLS-41b (48.0 g, 0.169 mol) was taken in clean and dry 1 Lit two neck RBF under argon atmosphere. Then add 491 mL of THF to RBF. Cool the RB in ice bath (0° C.). Add portion wise 1,1′—Carbonyldiimidazole (28.17 g, 0.174 mol) to RM for period of 10 min. The reaction mixture was stir at 15° C. for 12 h. TLC showed the reaction was completed, staring material was consumed and the product was formed (TLC—10% MeOH:EtOAc; TLC charring—Phosphomolybdic acid). After completion of reaction, solvent was dried and purified on silica gel column chromatography (100-200 mesh). The product was eluted with 50% ethyl acetate: hexane. Fraction containing product was evaporated to get 37.1 g (71% yield) of SOPL-WLS-41c as a white solid.

¹H NMR (400 MHz, CDCl₃): δ in ppm=4.33 (s, 1H), 3.40-3.43 (m, 4H), 3.17 (t, 2H, J=7.4 Hz), 1.50 (t, 2H, J=7.0 Hz), 1.25-1.30 (m, 28H), 0.88 (d, 3H, J=13.6 Hz).

MS: m/z calcd for C₁₉H₃₈N₂O ([M+H]⁺), 311.53; found 311.42.

### 3. Preparation of SOPL-WLS-41d

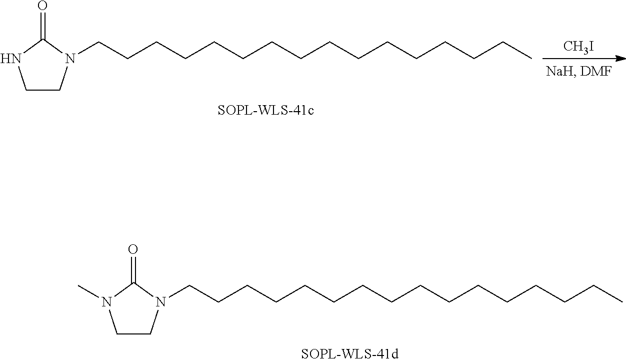

SOPL-WLS-41c (29.0 g, 0.093 mol) was taken in clean and dry 1 Lit two neck RBF under argon atmosphere. Then add 471 mL of dry DMF to RBF containing SM. Cool the RB in ice bath (Temp. 0° C.). Then, add portion wise 60% NaH (4.48 g, 0.112 mol) to RM for period of 15 min. at 0° C. and stir 30 min at same temp. Then add dropwise methyl iodide (17.4 mL, 0.281 mol) to the reaction mixture at 0° C. for duration of 15 min. Then allow the RM to rt and stir for 3 h. TLC showed the reaction was completed, staring material was consumed and the new spot was formed (TLC—EtOAc; TLC charring—Phosphomolybdic acid). After completion of reaction, reaction mixture was cool to 0° C. in ice bath and quenched with ice cold water (1 Lit). Then extracted with ethyl acetate (3×1000 mL). The organic layer was dried over sodium sulfate, filtered and concentrated to dryness. The crude product was purified by silica gel column chromatography (100-200 mesh). The product was eluted with 25%-35% ethyl acetate:hexane. The fraction containing product was evaporated to get 29.0 g (96% yield) of SOPL-WLS-41d as a white colour solid.

H NMR (500 MHz, CDCl₃): δ in ppm=3.27 (s, 4H), 3.16 (t, 2H, J=7.6 Hz), 2.78 (s, 3H), 1.48 (t, 2H, J=7.2 Hz), 1.29 (s, 7H), 1.25 (s, 22H), 0.88 (t, 3H, J=6.9 Hz).

MS: m/z calcd for C₂₀H₄₀N₂O ([M+H]⁺), 325.55; found 325.41.

### 4. Preparation of SOPL-WLS-41e

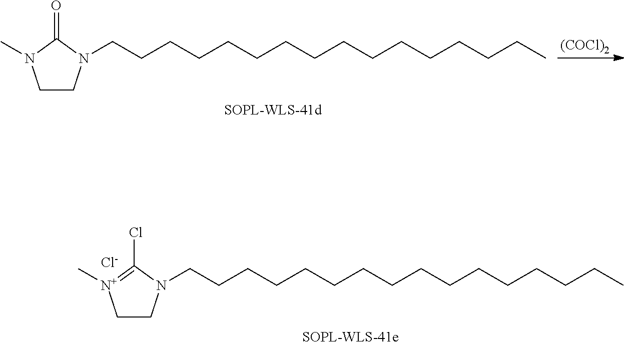

SOPL-WLS-41d (30.0 g, 0.092 mol) was taken in clean and dry 1 Lit two neck RBF under argon atmosphere. Then add 249 mL of dry toluene to RBF containing SM under argon atmosphere. After that add dropwise oxalyl chloride (118.9 mL, 1.386 mol) using addition funnel for a period of 30 min at rt. Then reaction mixture was heated to 65° C. for 72 hrs. After completion of reaction (TLC—ethyl acetate; TLC charring—Phosphomolybdic acid) solvent was evaporated to dryness to get crude compound. The crude compound was washed with cold ethyl acetate (2×100 mL) and dried to get 33.0 g of crude SOPL-WLS-41e as brown colour solid.

MS: m/z calcd for C₂₀H₄₀C₁₂N₂O ([M-Cl]⁺), 344.00; found 343.30.

### 5. Preparation of SOPL-WLS-41f

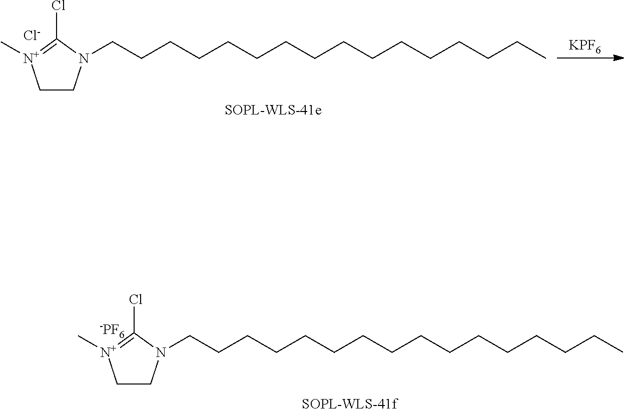

SOPL-WLS-41e (20.0 g, 0.053 mol) was taken in clean and dry 500 mL single neck RBF and dissolved in 115 mL DCM under argon atmosphere. Then added aq solution of KPF₆ (9.70 g, 0.053 mol, in 65 mL of water). Stir the reaction mixture at rt for 1 h. After completion of reaction (TLC—5% MeOH:DCM; TLC charring—Phosphomolybdic acid), the reaction mixture was poured into ice water, and extracted with DCM (2×400 mL). The combined organic layer washed with water (400 mL) and dried over sodium sulphate, filtered and evaporated to dryness. Then, residue was dissolved in DCM (70 mL) and product was precipitate by dropwise addition of diethyl ether (500 mL) under stirring. The solvent was decant and solid was dried under high vacuum to get 18.0 g (70% yield) of SOPL-WLS-41f as a white solid.

MS: m/z calcd for C₂₀H₄₀ClF₆N₂P ([M-PF₆]⁺), 344.00; found 343.34.

### 6. Preparation of SOPL-WLS-41

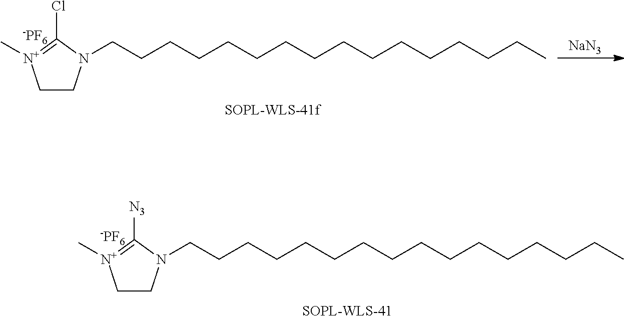

SOPL-WLS-41f (18.0 g, 0.037 mol) was taken in clean and dry 500 mL single neck RBF and dissolved in 90 mL of Dry MeCN under argon atmosphere. Then, added sodium azide (3.58 g, 0.055 mol) to the RM and stir at rt for 2.5 h. After completion of reaction (TLC—ethyl acetate; TLC charring—ninhydrin), reaction mixture was filtered through a pad of celite and washed with MeCN (20 mL). The organic layer was evaporated to dryness. The crude compound was dissolve in MeCN (70 mL) and precipitate by adding dropwise diethylether (500 mL). Solvent was decanted and solid was dried under high vacuum to get 14.1 g (77% yield) of SOPL-WLS-41 as a white solid.

¹H NMR (400 MHz, CDCl₃): δ in ppm=3.94-4.00 (m, 2H), 3.85-3.90 (m, 2H), 3.41 (t, 2H, J=7.6 Hz), 3.21 (s, 3H), 1.62 (t, 2H, J=7.1 Hz), 1.26 (s, 27H), 0.88 (t, 3H, J=6.8 Hz).

¹⁹F NMR (400 MHz, CDCl₃): δ in ppm=−73.35 and −75.24

MS: m/z calcd for C₂₀H₄₀F₆N₅P ([M-PF₆]⁺), 350.57; found 350.40.

### Synthesis of Azide (SOPL-WLS-97, n039)

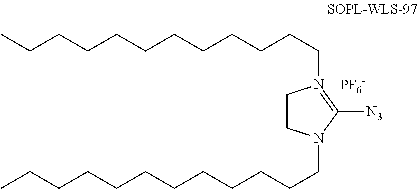

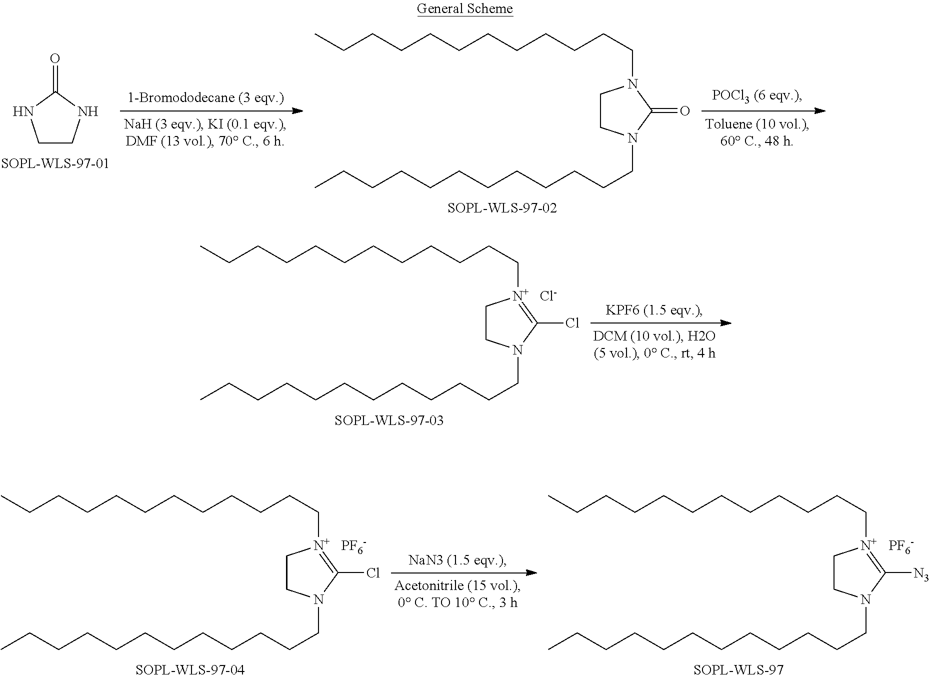

### 1. Preparation of 1,3-didodecylimidazolidin-2-one (SOPL-WLS-97-02)

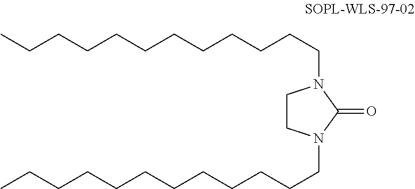 [SMILES: CCCCCCCCCCCCN1CCN(CCCCCCCCCCCC)C1=O]

To a solution of (SOPL-WLS-97-01) (20 g, 0.232 mol) in dry DMF (260 mL, 13 vol.) was added pinch of potassium iodide, followed by sodium hydride (27.9 g, 0.697 mol.), (60% dispersed in mineral oil) portion-wise over a period of 30 min. at 0° C. The mixture was allowed to warm to 65° C. and kept for 2 h. Then 1-bromododecane (167.2 mL, 0.697 mol) was added dropwise over a period of 30 mins at 65° C. and further stirred for 5 h. Progress of the reaction was monitored by TLC. Then reaction mixture was diluted with ice water (100 mL) at 0° C. and extracted with ethyl acetate (3×150 mL), washed with cool brine solution (2×100 mL), dried over Na₂SO₄ and concentrated under vacuum. The crude mass was purified by column chromatography over silica-gel (230-400 mesh), eluted in 10% EtOAc/Hexane to afford a pale yellow oil. (SOPL-WLS-97-02) (48 g, 50%). TLC Mobile phase details: 10% MeOH in DCM. ¹H NMR (400 MHz, DMSO-d₆): δ in ppm=4.24 (t, 1H, J1=5.2 Hz), 3.29 (d, 2H, J1=6.5 Hz), 3.12, (s, 3H), 2.93 (t, 3H, J1=7.1 Hz), 1.32 (m, 7H), 1.12 (m, 62H), 0.75 (m, 12H). MS: m z calcd for C₂₇H₅₄N₂O, 422.7; found 423.7 [M+H].

### 1. Preparation of 2-chloro-1,3-didodecyl-4,5-dihydro-1H-imidazol-3-ium (SOPL-WLS-97-03)

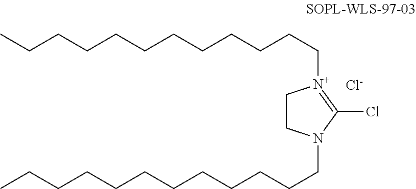 [SMILES: CCCCCCCCCCCCN1CC[N+](CCCCCCCCCCCC)=C1Cl ; [Cl-]]

To a stirred solution of (SOPL-WLS-97-02) (40 g, 0.0946 mol) in dry toluene (400 mL, 10 vol.) was added phosphorus chloride (44.2 mL, 0.4731 mol) dropwise at 0° C. Then reaction mixture was further stirred at 60° C. for 48 h. Progress of the reaction was monitored by TLC. Reaction mixture was concentrated under vacuum. The crude mass was stirred with diethyl ether (400 mL), filtered off, and dried under vacuum to afford a brownish solid (SOPL-WLS-97-03) (46 g, crude). TLC Mobile phase details: 7% MeOH in DCM. ¹H NMR (400 MHz, DMSO-d₆): δ in ppm=11.68 (s, 4H), 3.98 (s, 4H), 3.48 (t, 4H, J1=6.7 Hz), 3.21 (s, 1H), 3.02 (t, 1H, J1=6.8 Hz), 1.58 (s, 4H), 1.24 (s, 46H), 0.85 (d, 7H, J1=6.6 Hz). MS: m z calcd for C₂₇H₅₄ClN₂, 442.2; found 442.9 [M+].

### 2. Preparation of 2-chloro-1,3-didodecyl-4,5-dihydro-1H-imidazol-3-iumhexafluoro phosphate(V) (SOPL-WLS-97-04)

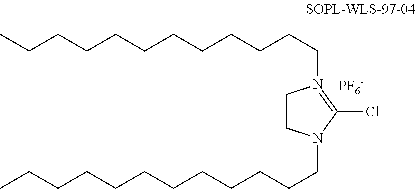

To an ice cool solution of (SOPL-WLS-97-03) (46 g, 0.1040 mol in DCM (460 mL, 10 vol.) was added a solution of KPF₆ (28 g, 0.1561 mol.) in water (230 mL, 5 vol.)) dropwise over a period of 50 min. at 0° C. Above reaction mixture was allowed to rt for 4 h. Progress of the reaction was monitored by TLC. Then the mixture was filtered through a celite bed, washed with DCM (2×80 mL), organic layer washed with water (2×60 mL), dried over Na₂SO₄ and concentrated under vacuum. The crude was washed with diethyl ether (150 ml×3) and dried under vacuum to aget an off white solid (SOPL-WLS-97-04) (32 g, 57%). TLC Mobile phase details: 7% MeOH in DCM. ¹H NMR (400 MHz, DMSO-d₆): δ in ppm=3.97 (s, 3H), 3.48 (t, 3H, J1=7.0 Hz), 3.20 (s, 1H), 3.02 (t, 1H, J1=7.0 Hz), 1.57 (s, 3H), 1.32 (m, 39H), 0.85 (m, 6H). MS: m z calcd for C₂₇H₅₄ClN₂, 442.2; found 442.8 [M⁺].

### 3. Preparation of 2-azido-1,3-didodecyl-4,5-dihydro-1H-imidazol-3-iumhexafluoro phosphate (V)) (SOPL-WLS-97)

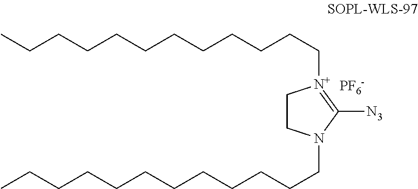

To a stirred solution of (SOPL-WLS-97-04) (32 g, 0.0546 mol) in acetonitrile (480 mL, 15 vol.) was added sodium azide (5.3 g, 0.0849 mol.) portion-wise over a period of 15 mins at 0° C. The mixture was further stirred at (0° C. to 10° C.) for 3 h. Progress of the reaction was monitored by TLC. Then reaction mixture was filtered through a celite bed, washed with acetonitrile (2×70 mL) and concentrated under vacuum. The solid was washed with diethyl ether (100 ml×2) and dried under vacuum to afford an off white solid. (SOPL-WLS-97) (20 g, 62%). TLC Mobile phase details: 7% MeOH in DCM. ¹H NMR (400 MHz, DMSO-d₆): δ in ppm=3.83 (s, 4H), 3.40 (t, 4H, J1=7.4 Hz), 1.58 (d, 4H, J1=6.3 Hz), 1.25 (s, 38H), 0.86 (t, 6H, J1=6.8 Hz). MS: m z calcd for C₂₇H₅₄N₅; 448.8; found 448.87 [M⁺].

### Synthesis for Azide (SOPL-WLS-42, n040)

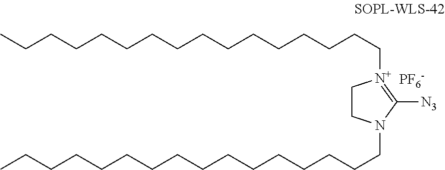

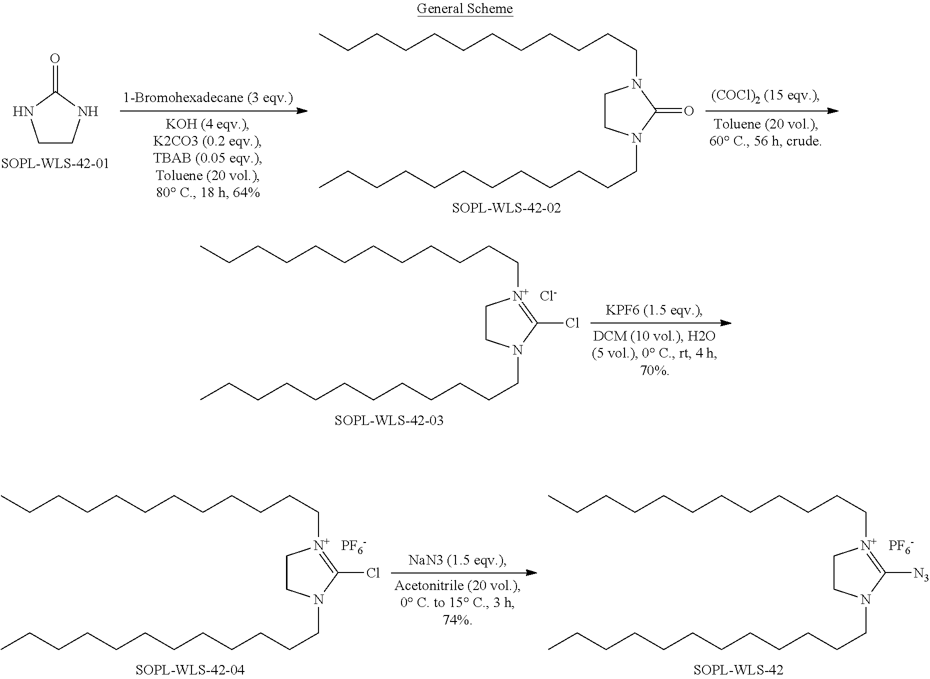

### 1. Preparation of 1,3-dihexadecylimidazolidin-2-one (SOPL-WLS-42-02)

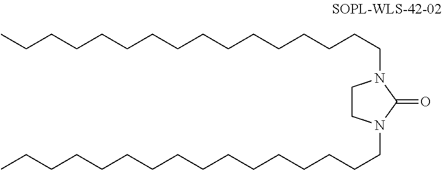 [SMILES: CCCCCCCCCCCCCCCCN1CCN(CCCCCCCCCCCCCCCC)C1=O]

To a stirred solution of (SOPL-WLS-42-01) (10 g, 0.1162 mol) in dry toluene (200 mL, 20 vol.) was added KOH (26 g, 0.4651 mol), K₂CO₃ (3.2 g, 0.0232 mol), TBAB (1.8 g, 0.00581 mol) and reaction mixture was stirred at rt for 30 mins. Then 1-bromo hexadecane (71 mL, 0.232 mol) was added dropwise over a period of 30 mins. The mixture was allowed to warm to 80° C. and kept for 16 h. Progress of the reaction was monitored by TLC. Then reaction mixture was diluted with ice water (80 mL) and extracted with ethyl acetate (2×100 mL), washed with brine solution (1×80 mL), dried over Na₂SO₄ and concentrated under vacuum. The crude mass was purified by column chromatography over silica-gel (230-400 mesh), eluted in 20% EtOAc/Hexane to afford an off white solid (SOPL-WLS-42-02) (40 g, 64%). TLC Mobile phase details: 20% EtOAc in Hexane. ¹H NMR (400 MHz, CDCl₃): δ in ppm=3.27 (s, 4H), 3.15 (t, 4H, J1=7.4 Hz), 1.48, (t, 4H, J1=6.8 Hz), 1.27, (d, 56H, J1=14.3 Hz), 0.88 (t, 6H, J1=6.9 Hz). MS: m z calcd for C₃₅H₇₀N₂O, 535; found 535.88 [M+H⁺]⁺.

### 2. Preparation of 2-chloro-1,3-dihexadecyl-4,5-dihydro-1H-imidazol-3-ium chloride (SOPL-WLS-42-03)

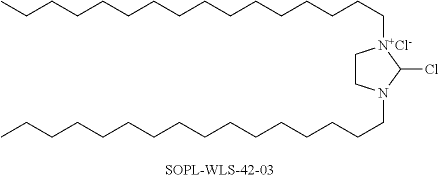

To a stirred solution of (SOPL-WLS-42-02) (20 g, 0.0373 mol) in dry toluene (400 mL, 20 vol.) was added oxalyl chloride (48.2 mL, 0.5607 mol) dropwise at 0° C. Then the mixture was further stirred at 60° C. for 56 h. Progress of the reaction was monitored by TLC. Reaction mixture was concentrated under vacuum. The crude mass was stirred with diethyl ether (300 mL), filtered off, and dried under vacuum to afford a brownish solid (SOPL-WLS-42-03) (24 g, crude). The crude material was directly used in next step without further purification. TLC Mobile phase details: 7% MeOH in DCM. ¹H NMR (400 MHz, CDCl₃): δ in ppm=4.33 (s, 4H), 3.64 (t, 4H, J1=7.2 Hz), 1.68, (s, 4H), 1.29, (m, 58H), 0.88 (m, 6H). MS: m z calcd for C₃₅H₇₀ClN₂, 554.4; found 555.06 [M].

### 3. Preparation of 2-chloro-1,3-dihexadecyl-4,5-dihydro-1H-imidazol-3-ium hexafluorophosphate (V) (SOPL-WLS-42-04)

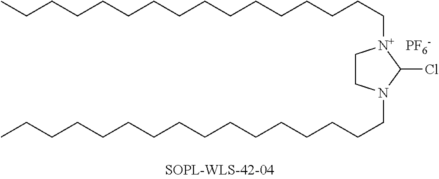

To an ice cool solution of (SOPL-WLS-42-03) (24 g, 0.0407 mol) in DCM (240 mL, 10 vol.) was added a solution of KPF₆ (11.24 g, 0.0611 mol.) in water (110 mL, 5 vol.) dropwise over a period of 25 min. at 0° C. Above reaction mixture was allowed to rt for 4 h. Progress of the reaction was monitored by TLC. Then the mixture was filtered through a celite bed, washed with DCM (2×60 mL), organic layer washed with water (2×50 mL), dried over Na₂SO₄ and concentrated under vacuum. The crude was washed with diethyl ether (100 ml×3) and dried under vacuum to a get an off white solid (SOPL-WLS-42-04) (20 g, 70%). TLC Mobile phase details: 7% MeOH in DCM. ¹H NMR (400 MHz, CDCl₃): δ in ppm=4.10 (s, 4H), 3.54 (t, 4H, J1=7.6 Hz), 1.65, (t, 4H, J1=7.0 Hz), 1.28 (m, 56H, J1=20.5 Hz), 0.88 (m, 6H, J1=6.9 Hz). MS: m z calcd for C₃₅H₇₀ClN₂, 554.4; found 555.94 [M].

### 4. Preparation of -azido-1,3-dihexadecyl-4,5-dihydro-1H-imidazol-3-ium hexafluorophosphate (V) (SOPL-WLS-42)

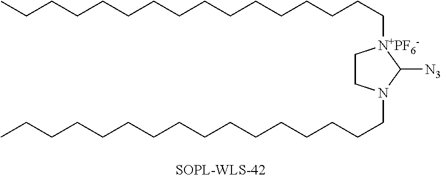

To a stirred solution of (SOPL-WLS-42-04) (20 g, 0.0286 mol) in acetonitrile (400 mL, 20 vol.) was added sodium azide (3.72 g, 0.0572 mol.) portion-wise over a period of 10 mins at 0° C. The mixture was further stirred at (0° c. to 10° C.) for 3 h. Progress of the reaction was monitored by TLC. Then reaction mixture was filtered through a celite bed, washed with acetonitrile (2×50 mL) and concentrated under vacuum. The solid was washed with diethyl ether (80 ml×2) and dried under vacuum to afford an off white solid. (SOPL-WLS-42) (15 g, 74%). TLC Mobile phase details: 5% MeOH in DCM. ¹H NMR (400 MHz, CDCl₃): δ in ppm=3.92 (s, 4H), 3.45 (t, 4H, J1=7.7 Hz), 1.64, (t, 4H, J1=7.1 Hz), 1.28 (m, 54H), 0.88 (s, 6H, J1=6.9 Hz). MS: m z calcd for C₃₅H₇₀N₅, 561.0; found 561.48 [M].

### Synthesis of SOPL-WLS-70

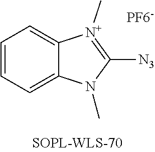

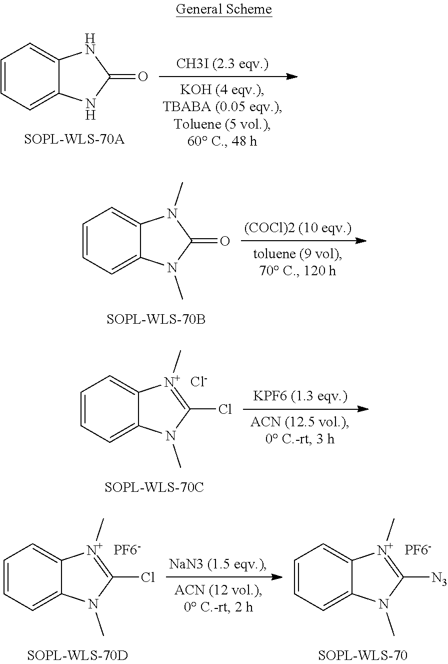

### 1. Preparation of 1,3-dimethyl-1,3-dihydro-2H-benzo (d)imidazole-2-one (SOPL-WLS-70B)

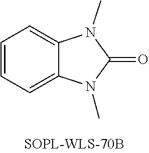

To a mixture of 1,3-dihydro-2H-benzo(d)imidazole-2-one (30 g, 0.22 mol) in toluene (150 mL, 5 vol.) was added TBAB (3.6 g, 0.01 mol.), 40% KOH solution (50.14 g, 0.89 mol.). Then methyl iodide (32 mL, 0.51 mol) was added dropwise over a period of 30 mins at RT, stirred at 60° C. for 48 h. Progress of the reaction was monitored by TLC. Above reaction was extracted with ethyl acetate (3×100 mL) washed with 1N HCl (2×50 mL), sat. NaHCO₃ (2×50 mL). Combined organics were dried over Na₂SO₄, concentrated under reduced pressure to afford the crude which was purified by column chromatography over silica gel (230-400 mesh) eluted in 1% MeOH/DCM to afford compound (SOPL-WLS-70B) (27 g, 75%) as a pale yellow solid. TLC Mobile phase details: 70% EtOAC/Hexane.

¹H NMR (500 MHz, DMSO-d₆): δ in ppm=7.02 (m, 2H), 7.07 (m, 2H), 3.32 (s, 6H). MS: m z calcd. for C₉H₁₀N₂O 162.19; found 163.13 (M+H⁺).

### 2. Preparation of 2-Chloro-1, 3-dimethyl-1H-benzo (d) imidazole-3-ium (SOPL-WLS-70C)

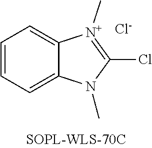

To a cool stirred solution of (SOPL-WLS-70B) (27 g, 0.16 mol) in toluene (247 mL) was added oxalyl chloride (145 mL, 1.68 mol.) dropwise over a period of 40 mins under argon atmosphere. Reaction mixture was stirred at 70° C. for 5 days. Progress of the reaction was monitored by TLC. A solid precipitated was observed upon cooling at 0° C. for 3 h. the solid was filtered and washed with cold toluene (3×40 ml), dried under vacuum to afford compound (SOPL-WLS-70C) (21 g, 68%) as an off-white solid. TLC Mobile phase details: 10% MeOH in DCM. ¹H NMR (500 MHz, DMSO-d₆): δ in ppm=8.09 (q, 2H, J=3 Hz), 7.72 (q, 2H, J=3.2 Hz), 4.05 (s, 6H). MS: m z calcd for C₉H₁₀N₂Cl 181.64; found 181.10 (M)+.

### 3. Preparation of 2-Chloro-1, 3-dimethyl-1H-benzo (d) imidazole-3-ium hexafluorophosphate (SOPL-WLS-70D)

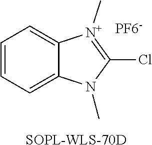

To a stirred solution of (SOPL-WLS-70C) (21 g, 0.09 mol) in acetonitrile (262 mL, 12.5 vol.) was added KPF₆ (23.2 g, 0.12 mol.) portion-wise over a period of 30 mins at 0° C. Above reaction mixture was stirred at RT for 3 h. Progress of the reaction was monitored by TLC. Then the mixture was filtered through a celite bed, washed with acetonitrile (2×40 mL) and evaporated under reduced pressure to get a crude solid. The crude was re-dissolved in acetonitrile (15 ml), then was added to a precool diethyl ether (120 mL) drop-wise at −78° C. under stirring. Solid precipitated was filtered off and washed with ether (2×45 mL) and dried to get the desired compound (SOPL-WLS-70C) (23 g, 72%) as an off-white solid. TLC Mobile phase details: 7% MeOH in DCM. ¹H NMR (500 MHz, DMSO-d6): δ in ppm=8.07 (td, 2H, J1=6.5 Hz, J2=3.2 Hz), 7.71 (m, 2H), 4.04 (s, 6H). MS: m z calcd for C₉H₁₀N₂Cl: 181.64; found 181.15 (M)⁺.

### 4. Preparation of 2-azido-1, 3-dimethyl-1H-benzo[d]imidazole-3-ium (SOPL-WLS-70)

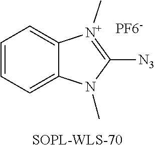

To an ice-cool stirred solution of (SOPL-WLS-70D) (23 g, 0.07 mol) in acetonitrile (276 mL, 12 vol.) was added sodium azide (6.87 g, 0.10 mol.) portion-wise over a period of 20 mins. Above reaction mixture was stirred at RT for 3 h. Progress of the reaction was monitored by TLC. Then the mixture was filtered through a celite bed, washed with acetonitrile (2×50 mL) and evaporated under reduced pressure to give a crude solid. The crude was re-dissolved in acetonitrile (20 ml), then was added to a precool diethyl ether (120 mL) drop-wise at −78° C. under stirring. The solid was precipitated out which was filtered off and washed with ether (2×45 mL), and dried under vacuum to get the desired compound (SOPL-WLS-70) (18 g, 76%) as a yellow solid. TLC Mobile phase details: 100% Ethyl acetate. ¹H NMR (500 MHz, DMSO-d₆): δ in ppm=7.94 (m, 2H,) 7.64 (td, 2H, J1=6.5 Hz, J₂=3.2 Hz), 3.97 (s, 6H). MS: m z calcd for C₉H₁₀N₅⁺PF₆⁻ 188.21; found 188.07 (M+). ¹⁹F NMR (500 MHz, DMSO-d₆): δ in ppm=−69.32, −70.33. IR (KBr)=2189 cm⁻¹.

### Synthesis of Azide (SOPL-WLS-96, n071)

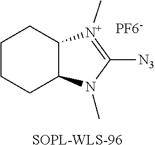

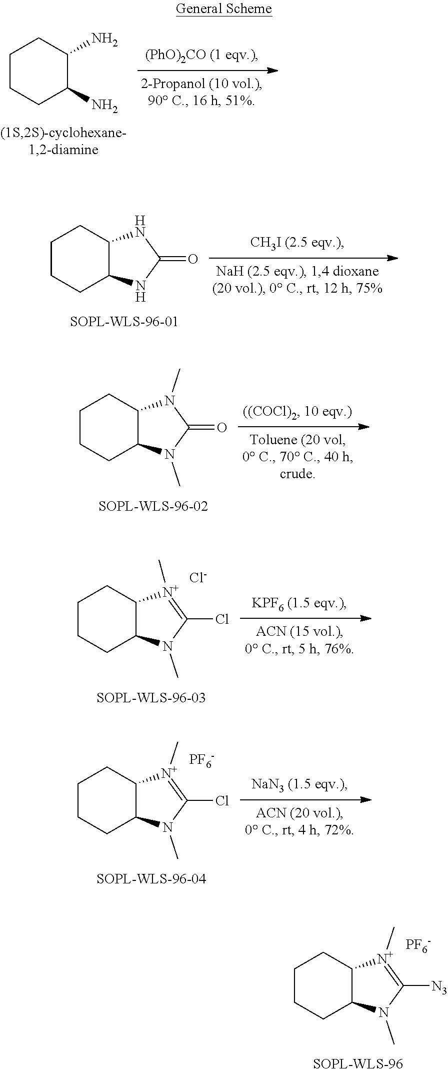

### 1. Preparation of (3aS,7aS)-octahydro-2H-benzo[d]imidazol-2-one (SOPL-WLS-96-01)

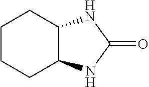

To a stirred solution of (1S, 2S)-cyclcohexane-1, 2-diamine (11 g, 0.0964 mol) in 2-propanol (110 mL, 10 vol.), was added diphenyl carbonate (16.3 g, 0.0767 mol) at rt under argon atmosphere. Then the reaction mixture was allowed to 90° C. for 3 h. Progress of the reaction was monitored by TLC. Solvent was evaporated under reduced pressure to afford a gummy syrup. The gummy mass was purified by column chromatography over silica gel (230-400 mesh) eluted in 2% MeOH/DCM to get as an off white solid (SOPL-WLS-96-01) (7 g, 51%). TLC Mobile phase details: 7% MeOH in DCM. ¹H NMR (500 MHz, DMSO-d₆): δ in ppm=6.32 (s, 2H), 2.86 (q, 2H, J1=2.5 Hz), 1.82 (m, 2H), 1.68 (q, 2H, J1=1.8 Hz), 1.28 (d, 2H, J1=4.1 Hz). MS: m z calcd for C₇H₁₂N₂O, 140.02; found 140.92 [M+H⁺].

### 2. (Preparation of 3aS,7aS)-1,3-dimethyloctahydro-2H-benzo[d]imidazol-2-one (SOPL-WLS-96-02)

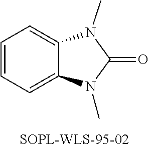

To a stirred solution of (SOPL-WLS-96-01) (11.5 g, 0.0821 mol) in dry 1,4 dioxan (230 mL, 20 vol.) was added NaH (60%) (8.2 g, 0.205 mol.), portion-wise at 10° C. and the reaction mixture was further stirred at 65° C. for 3 h. Then Iodomethane (12.7 mL, 0.205 mol) was added dropwise over a period of 20 min. at 0° C. Above mixture was allowed to rt for 12 h. Progress of the reaction was monitored by TLC. Then the mixture was quenched with ice water (100 ml), extracted with DCM (3×100 mL), washed with brine (80 ml×1) solution, dried over Na₂SO₄ and concentrated under vacuum to afford as a brown syrup. The syrup was purified by column chromatography over silica gel (230-400 mesh) eluted in 2% MeOH/DCM to get as a light yellow syrup. (SOPL-WLS-95-02) (10.5 g, 76%). TLC Mobile phase details: 7% MeOH in DCM; ¹H NMR (500 MHz, DMSO-d₆): δ in ppm=2.56 (s, 6H), 2.53 (m, 2H), 1.98 (dd, 2H, J1=11.0 Hz, J2=2.1 Hz,), 1.78 (m, 2H), 1.34 (m, 2H), 1.23 (m, 2H). MS: m z calcd for C₉H₁₆N₂O, 168.2; found 169.18 [M+H⁺].

### 3. Preparation of (3aS,7aS)-2-chloro-1,3-dimethyl-3a,4,5,6,7,7a-hexahydro-1H-benzo[d]imidazol-3-iumchloride (SOPL-WLS-96-03)

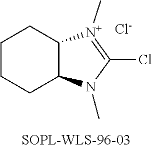

To a stirred solution of (SOPL-WLS-96-02) (10 g, 0.059 mol) in dry toluene (100 mL, 10 vol) was added oxalyl chloride (51.07 mL, 0.592 mol) dropwise at 0° C. and reaction mixture was further stirred at 70° C. for 40 h. Progress of the reaction was monitored by TLC. Then reaction mixture was concentrated under reduced pressure to afford a brown syrup; which was washed with n-pentane (50 ml×3), diethyl ether (120 ml×3) and dried under vacuum to afford (SOPL-WLS-96-03) as a brown syrup (14 g). the crude was used as such in next step. TLC Mobile phase details: 7% MeOH in DCM. ¹H NMR (500 MHz, DMSO-d₆): δ in ppm=14.35 (s, 1H), 8.38 (s, 1H), 2.57 (s, 6H), 2.53 (m, 2H), 1.99 (m, 2H), 1.80 (m, 2H), 1.33 (m, 2H), 1.22 (m, 2H). MS: m z calcd for C₉H₁₆ClN₂, 187.7; found 188.65 [M+H⁺].

### 4. Preparation of (3aS,7aS)-2-chloro-1,3-dimethyl-3a,4,5,6,7,7a-hexahydro-1H-benzo[d]imidazol-3-ium hexafluorophosphate (V) (SOPL-WLS-96-04)

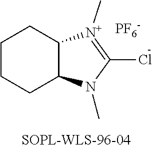

To a stirred solution of (SOPL-WLS-96-03) (14 g, 0.627 mol) in dry ACN (280 mL, 20 vol.) was added KPF₆ (17.5 g, 0.0941 mol.) portion wise over a period of 20 mins at 0° C. Above reaction mixture was stirred at rt for 5 h. Progress of the reaction was monitored by TLC. Then the mixture was filtered through a celite bed washed with ACN (2×50 mL), dried over Na₂SO₄ and concentrated under vacuum to get a gummy mass. The syrup was treated with diethyl ether and a solid precipitation was observed. The solid was off, washed with diethyl ether (300 mL) and dried under vacuum to afford (SOPL-WLS-96-04) as a brown solid. (16 g, 76%). TLC Mobile phase details: 7% MeOH in DCM. ¹H NMR (500 MHz, DMSO-d₆): δ in ppm, 2.57 (s, 6H), 2.54 (t, 2H, J1=2.8 Hz), 1.99 (d, 2H, J1=11.0 Hz), 1.78 (t, 2H, J1=10.3 Hz), 1.33 (m, 2H), 1.23 (m, 2H). MS: m z calcd for C₉H₁₆ClN₂, 187.65, found 188.72 [M+H⁺].

### 5. Preparation of ((3aS,7aS)-2-azido-1,3-dimethyl-3a,4,5,6,7, 7a-hexahydro-1H-3l4-benzo[d]imidazole hexafluorophosphate (V) (SOPL-WLS-96)

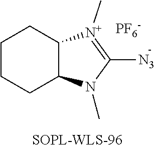

To a stirred solution of (SOPL-WLS-96-04) (16 g, 0.0481 mol) in dry ACN (320 mL, 20 vol.) was added NaN₃ (4.7 g, 0.0722 mol.) portion wise over a period of 20 mins at 0° C. and reaction mixture was stirred at rt for 4 h. Progress of the reaction was monitored by TLC. Then the mixture was filtered through a celite bed washed with ACN (2×70 mL), dried over Na₂SO₄ and concentrated under vacuum to get brownish solid. The solid was filtered off and washed with diethyl ether (90 ml×4), dried under vacuum to afford (SOPL-WLS-96) as a brown solid. (12 g, 72%). TLC Mobile phase details: 7% MeOH in DCM. ¹H NMR (500 MHz, DMSO-d₆): δ in ppm, 3.27 (t, 2H, J1=3.8 Hz), 3.05 (s, 6H), 2.21 (d, 2H, J1=9.0 Hz), 1.85 (d, 2H, J1=6.9 Hz), 1.35 (m, 4H). MS: m z calcd for C₉H₁₆N₅, 194.3, found 194.01 [M⁺].

### Synthesis of Azide (SOPL-WLS-95)

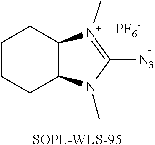

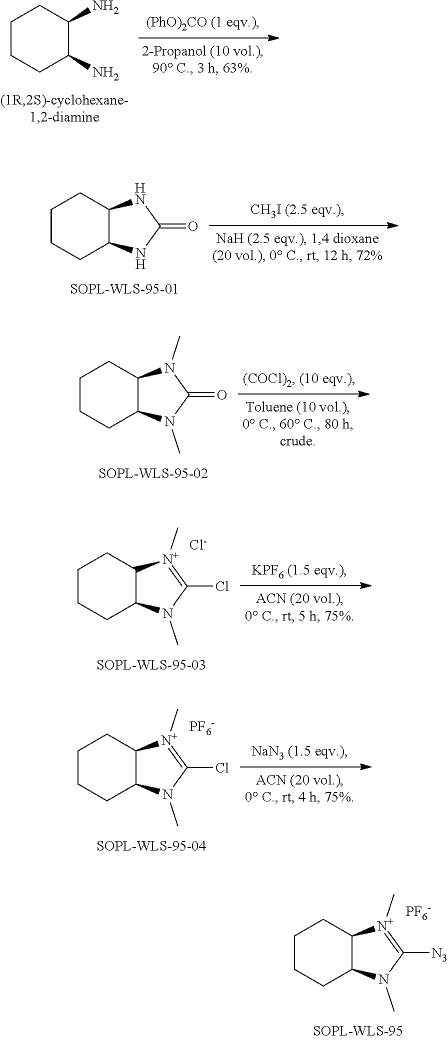

### 1. Preparation of (3aR,7aS)—Octahydro-2H-benzo[d]imidazol-2-one (SOPL-WLS-95-01)

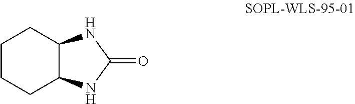

To a stirred solution of (1R, 2S)-cyclcohexane-1, 2-diamine (30 g, 0.263 mol) in 2-propanol (300 mL, 10 vol) was added diphenyl carbonate (54 g, 0.22 mol) at rt under argon atmosphere. Then the reaction mixture was allowed to 90° C. for 3 h. Progress of the reaction was monitored by TLC. Solvent was evaporated under reduced pressure to afford a gummy syrup. The syrup was purified by column chromatography over silica gel (230-400 mesh) eluted in 3% MeOH/DCM to get an off white solid (SOPL-WLS-95-01) (23 g, 63%). TLC Mobile phase details: 7% MeOH in DCM. ¹H NMR (400 MHz, DMSO-d₆): δ in ppm=6.14 (s, 2H), 3.44 (m, 2H), 1.57 (m, 2H), 1.42 (m, 4H), 1.22 (m, 2H). MS: m z calcd for C₇H₁₂N₂O, 140.02; found 140.92 ([M+H]).

### 2. Preparation of (3aR,7aS)-1,3-dimethyloctahydro-2H-benzo[d]imidazol-2-one (SOPL-WLS-95-02)

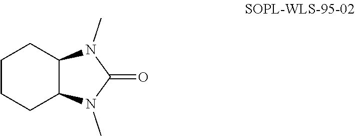

To a stirred solution of (SOPL-WLS-95-01) (24 g, 0.1714 mol) in 1,4 dioxan (480 mL, 20 vol.) was added NaH (60% dispersion in mineral oil)) (17.1 g, 0.4285 mol.) portion-wise over a period of 30 mins at 10° C. Then the mixture was allowed to 65° C. and kept for 3 h. After that the mixture was cool to 0° C. and Iodomethane (26.7 mL, 0.4285 mol) was added dropwise. Further, the mixture was stirred at rt for 12 h. Progress of the reaction was monitored by TLC. Then the mixture was quenched with ice water (150 ml), extracted with DCM (3×100 mL), washed with brine (50 ml×2) solution, dried over Na₂SO₄ and concentrated under vacuum to afford a brownish syrup. The syrup was purified by column chromatography over silica gel (230-400 mesh) eluted in 2% MeOH/DCM to get as a light yellow syrup. (SOPL-WLS-95-02) (21 g, 72%). TLC Mobile phase details: 7% MeOH in DCM; ¹H NMR (500 MHz, DMSO-d₆): δ in ppm=3.28 (m, 2H), 2.58 (s, 6H), 1.71 (qd, 2H, J1=8.7 Hz, J2=4.6 Hz,), 1.46 (m, 2H), 1.37 (m, 2H), 1.27 (m, 2H). MS: m z calcd for C₉H₁₆N₂O, 168.2; found 169.18 ([M+H⁺]).

### 3. Preparation of (3aR,7aS)-2-chloro-1,3-dimethyl-3a,4,5,6,7,7a-hexahydro-1H-benzo[d]imidazol-3-ium chlorid (SOPL-WLS-95-03)

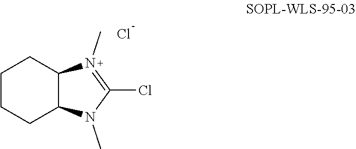

To a stirred solution of (SOPL-WLS-95-02) (24 g, 0.1428 mol) in toluene (240 mL, 10 vol) was added oxalyl chloride (122.29 mL, 1.428 mol) dropwise at 0° C., the mixture was further stirred at 60° C. for 80 h. Progress of the reaction was monitored by TLC. Then reaction mixture was concentrated under reduced pressure to afford a crude mass; which was washed with n-pentane (100 ml×2), diethyl ether (100 ml×3) and dried under vacuum to afford (SOPL-WLS-95-03) as a brownish syrup (25 g). TLC Mobile phase details: 7% MeOH in DCM. ¹H NMR (500 MHz, DMSO-d₆): δ in ppm=10.85 (s, 4H), 3.30 (m, 2H), 2.58 (s, 6H), 1.71 (qd, 2H, J1=8.7 Hz, J2=4.5 Hz,), 1.46 (m, 2H), 1.37 (m, 2H), 1.28 (m, 2H). MS: m z calcd for C₉H₁₆ClN₂, 187.7; found 188.72 ([M+H⁺]).

### 4. Preparation of (3aR,7aS)-2-chloro-1,3-dimethyl-3a,4,5,6,7,7a-hexahydro-1H-benzo[d]imidazol-3-iumhexafluorophosphate (V) (SOPL-WLS-95-04)

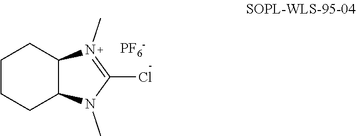

To a stirred solution of (SOPL-WLS-95-03) (25 g, 0.1125 mol) in ACN (500 mL, 20 vol.) was added KPF₆ (31.27 g, 0.1685 mol.) portion wise over a period of 40 mins at 0° C. Then the reaction mixture was stirred at rt for 5 h. Progress of the reaction was monitored by TLC. After that the mixture was filtered through a celite bed washed with ACN (2×80 mL), dried over Na₂SO₄ and concentrated under vacuum to get crude syrup. The syrup was washed with diethyl ether (150 ml×3) and dried under vacuum afford (SOPL-WLS-95-04) as a brown solid. (28 g, 75%). TLC Mobile phase details: 7% MeOH in DCM. ¹H NMR (500 MHz, DMSO-d₆): δ in ppm 3.29 (t, 2H, J1=3.8 Hz), 2.58 (s, 6H), 1.71 (m, 2H), 1.46 (m, 2H), 1.38 (m, 2H), 1.27 (tt, 2H, J1=11.1 Hz, J2=3.9 Hz). MS: m z calcd for C₉H₁₆ClN₂, 187.65, found 187.7 ([M+H⁺])

### 5. Preparation of (3aR,7aS)-2-azido-1,3-dimethyl-3a,4,5,6,7,7a-hexahydro-1H-314-benzo[d]imidazole hexafluorophosphate (V) (SOPL-WLS-95)

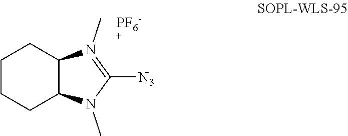

To a stirred solution of (SOPL-WLS-95-04) (16 g, 0.0481 mol) in ACN (320 mL, 20 vol.) was added NaN₃ (4.69 g, 0.0722 mol.) portion wise over a period of 30 mins at 0° C. and reaction mixture was stirred at rt for 4 h. Progress of the reaction was monitored by TLC. Then the mixture was filtered through a celite bed washed with ACN (2×100 mL), dried over Na₂SO₄ and concentrated under vacuum to get crude syrup. The syrup was washed with diethyl ether (60 ml×3) and dried under vacuum afford (SOPL-WLS-95) as a light yellow solid. (12 g, 74%). TLC Mobile phase details: 7% MeOH in DCM. ¹H NMR (400 MHz, DMSO-d₆): δ in ppm 4.03 (m, 2H), 3.05 (s, 6H), 1.76 (m, 4H), 1.36 (m, 4H). MS: m/z calcd for C₉H₁₆N₅, 194.3, found 193.97 ([M+])

### Synthesis of (SOPL-WLS-94)

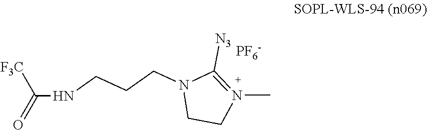

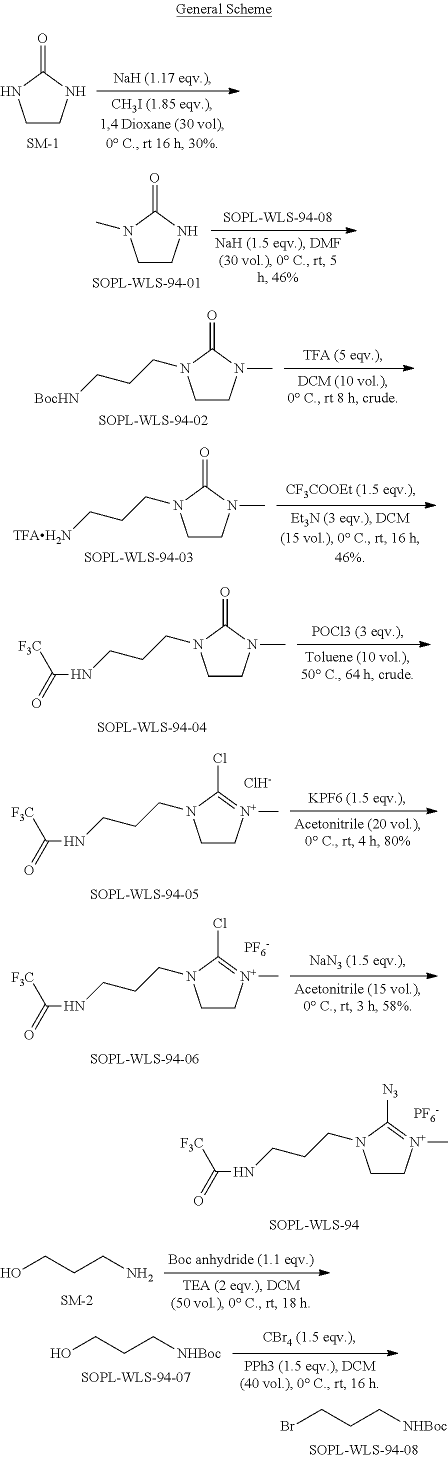

### 1. Preparation of 1-methylimidazolidin-2-one (SOPL-WLS-94-01)

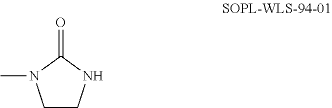

To a solution of (SM-1) (50 g, 0.5813 mol) in 1, 4-dioxane (1.2 L, 30 vol.) was added sodium hydride (60% dispersion in mineral oil) (27.2 g, 0.6802 mol.) portion-wise at 0° C. Then the mixture was allowed to stir at 65° C. for 3 h. After that the mixture was cool to 0° C. and Iodomethane (66.8 mL, 1.074 mol) was added dropwise over a period of 50 mins. Further, the mixture was allowed to rt and kept for 16 h. Progress of the reaction was monitored by TLC. Above mixture was then filtered through a celite bed, washed with DCM (3×100 mL). Filtrate was concentrated under reduced pressure to afford a thick syrup. The syrup was purified by column chromatography over silica gel (230-400 mesh) eluted in 2% MeOH/DCM to get an off-white solid (SOPL-WLS-94-01) (17.5 g, 30%). TLC Mobile phase details: 10% MeOH in DCM. ¹H NMR (500 MHz, DMSO-d₆): δ in ppm=6.26 (s, 1H), 3.27 (m, 2H), 3.19 (dd, 2H, J1=8.6 Hz, J2=6.5 Hz), 2.60 (s, 3H). MS: m z calcd for C₄H₈N₂O, 100.1; found 101.08 ([M+H]).

### 2. Preparation of tert-butyl (3-(3-methyl-2-oxoimidazolidin-1-yl)propyl)carbamate) (SOPL-WLS-94-02)

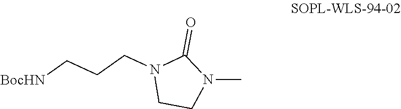 [SMILES: CN1CCN(CCCNC(=O)OC(C)(C)C)C1=O]

To a stirred solution of (SOPL-WLS-94-01) (20 g, 0.2 mol) in 1, 4 dioxan (1 L, 20 vol.) was added sodium hydride (60% dispersion in mineral oil) (12 g, 0.3 mol.) portion-wise over a period of 30 min at 10° C. The mixture was further stirred at 65° C. for 3 h. After that the reaction mixture was cool to 0° C. and a solution of alkyl bromide (71. g, 0.3 mol) in 1, 4 dioxan (200 mL) was added dropwise. Above reaction was stirred at rt for 5 h. Progress of the reaction was monitored by TLC. Then reaction mixture was diluted with ice water (150 mL) and extracted with ethyl acetate (3×200 mL), dried over Na₂SO₄ and concentrated under reduced pressure to get a gummy mass. The crude was purified by column chromatography over silica-gel (230-400 mesh) eluted in 2% MeOH/DCM to afford a pale yellow oil (SOPL-WLS-94-02) (26 g, 50%). %). TLC Mobile phase details: 10% MeOH in DCM. ¹H NMR (400 MHz, DMSO-d₆): δ in ppm=6.75 (d, 1H, J1=5.4 Hz), 3.21 (s, 4H), 3.15, (m, 1H), 3.03 (t, 2H, J1=7.1 Hz), 2.89 (q, 2H, J1=6.6 Hz), 2.63 (s, 3H), 1.52 (m, 2H), 1.37 (s, 9H). MS: m z calcd for C₁₂H₂₃N₃O₃, 257.3; found 258.08 ([M+H])

### 3. Preparation of 1-methyl-3-(3-((2,2,2-trifluoroacetyl)-l4-azaneyl)propyl)imidazolidin-2-one (SOPL-WLS-94-03)

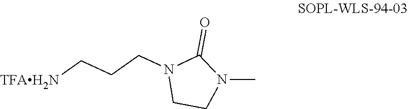

To a stirred solution of (SOPL-WLS-94-03) (29 g, 0.1124 mol) in DCM (290 mL, 10 vol.) was added trifluoroacetic acid (43.3 mL, 0.562 mol.) dropwise at 0° C. Above reaction mixture was stirred at rt for 8 h. Progress of the reaction was monitored by TLC. Then solvent was reduced under reduced pressure, co-distilled with toluene (2×100 mL) and dried to afford a pale yellow gummy mass (SOPL-WLS-94-01) (30 g, crude). The crude was directly used in next step without further purification. TLC Mobile phase details: 10% MeOH in DCM. ¹H NMR (400 MHz, DMSO-d₆): δ in ppm=10.05 (s, 2H), 7.73 (s, 4H), 3.22 (d, 4H, J1=8.6 Hz), 3.13 (t, 2H, J1=6.9 Hz), 2.76 (m, 2H), 2.64 (d, 3H, J1=6.9 Hz), 1.71 (m, 2H). MS: m z calcd for C₇H₁₅N₃O, 157.2; found 158.06 ([M+H]).

### 4. Preparation of 2,2,2-trifluoro-N-(3-(3-methyl-2-oxoimidazolidin-1-yl)propyl)acetamide) (SOPL-WLS-94-04)

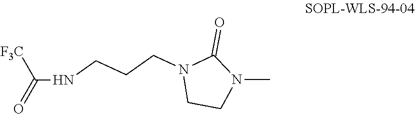 [SMILES: CN1CCN(CCCNC(=O)C(F)(F)F)C1=O]

To a cool stirred solution of (SOPL-WLS-94-03) (26 g, 0.10230 mol) in DCM (390 mL, 15 vol.) was added triethylamine (41.9 mL, 0.3073 mol.) dropwise. Then ethyl trifluoroacetate (18.16 mL, 0.1535 mol.) was added dropwise over a period of 20 mins, at 0° C. Above reaction mixture was stirred at rt for 16 h. Progress of the reaction was monitored by TLC. Then the reaction mass was diluted with ice water (150 mL) and extracted with DCM (2×200 mL), dried over Na₂SO₄ and concentrated under reduced pressure. The crude was purified by column chromatography over silica-gel (100-200 mesh) eluted in 5% MeOH in DCM to afford an off-white solid (SOPL-WLS-94-04) (14 g, 51% for 2 steps). TLC Mobile phase details: 10% MeOH in DCM. ¹H NMR (400 MHz, DMSO-d₆): δ in ppm=9.41 (s, 1H), 3.23 (m, 4H), 3.18, (m, 2H), 3.07 (t, 2H, J1=7.1 Hz), 2.64 (d, 3H, J1=2.1 Hz), 1.6 (m, 2H). MS: m z calcd for C₉H₁₄F₃N₃O₂, 253.2; found 254.08 ([M+H])

### 5. Preparation of N-(3-(2-chloro-3-methyl-4,5-dihydro-1H-3l4-imidazol-1-yl)propyl)-2,2,2-trifluoroaceta midechlorine (SOPL-WLS-94-05)

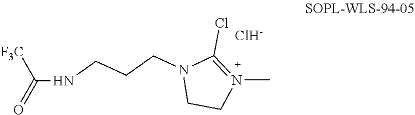

To a stirred solution of (SOPL-WLS-94-04) (14 g, 0.0054 mol) in Toluene (280 mL, 20 vol) was added phosphorus chloride (15.3 mL, 0.1634 mol) dropwise at 0° C. Then reaction mixture was further stirred at 50° C. for 64 h. Progress of the reaction was monitored by TLC. Then reaction mixture was concentrated under reduced pressure to afford a crude mass; which was washed with diethyl ether (100 ml×3) and dried under vacuum to afford as a yellowish solid (SOPL-WLS-94-05) (15 g, crude). TLC Mobile phase details: 7% MeOH in DCM. ¹H NMR (500 MHz, DMSO-d₆): δ in ppm=12.2 (s, 2H), 9.45 (s, 1H), 3.23 (m, 4H), 3.17 (q, 2H, J1=6.2 Hz), 3.07 (t, 2H, J1=7.2 Hz), 2.64 (s, 3H), 1.66 (m, 2H). MS: m z calcd for C₉H₁₄C₁F₃N₃O, 272.7; found 273.74 ([M+H]).

### 6. Preparation of N-(3-(2-chloro-3-methyl-4,5-dihydro-1H-3l4-imidazol-1-yl)propyl)-2,2,2-trifluoroacetamide) (SOPL-WLS-94-06)

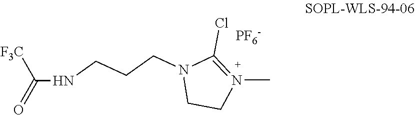

To a cool solution of (SOPL-WLS-94-05) (16 g, 0.052130 mol in ACN (320 mL, 20 vol.) was added KPF₆ (14.5 g, 0.0781 mol.) portion wise over a period of 30 mins at 0° C. Above reaction mixture was stirred at rt for 4 h. Progress of the reaction was monitored by TLC. Then the mixture was filtered through a celite bed washed with ACN (2×80 mL), dried over Na₂SO₄ and concentrated under reduced pressure to get a crude mass; which was washed with diethyl ether (150 ml×3) and dried under vacuum to afford as a yellowish solid (SOPL-WLS-94-06) (18 g, 80%). TLC Mobile phase details: 10% MeOH in DCM.

¹H NMR (500 MHz, DMSO-d₆): δ in ppm=11.45 (d, 2H, J1=687.9 Hz), 9.42 (s, 1H), 3.21, (d, 4H, J1=13.8 Hz), 3.17 (q, 2H, J1=6.2 Hz), 3.07 (t, 2H, J1=6.9 Hz), 2.64 (s, 3H), 2.07 (s, 3H), 1.66 (m, 2H). MS: m z calcd for C₉H₁₄C₁F₃N₃O, 272.7; found 273.73 ([M+H])

### 7. Preparations of N-(3-(2-azido-3-methyl-4,5-dihydro-1H-3l4-imidazol-1-yl)propyl)-2,2,2-trfluoroacetamide hexafluoro phosphate (V) (SOPL-WLS-94)

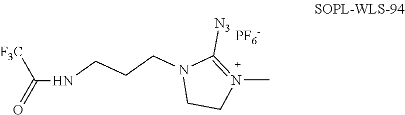

To a stirred solution of (SOPL-WLS-94-06) (18 g, 0.0431 mol) in acetonitrile (360 mL, 20 vol.) was added sodium azide (4.2 g, 0.0647 mol.) portion-wise over a period of 20 mins at 0° C. and further stirred at rt for 3 h. Progress of the reaction was monitored by TLC. Then reaction mixture was filtered through a celite bed washed with acetonitrile (2×80 mL) and concentrated under reduced pressure to afford a light yellow solid. The solid was washed with diethyl ether (80 ml×4) and dried under vacuum to afford as an off white solid. (SOPL-WLS-94) (12 g, 61%). TLC Mobile phase details: 7% MeOH in DCM. ¹H NMR (500 MHz, DMSO-d₆): δ in ppm=9.49 (s, 1H), 3.81 (m, 4H), 3.38 (t, 2H, J1=7.2 Hz), 3.24 (q, 2H, J1=6.4 Hz), 3.13 (s, 3H), 1.8 (m, 2H). MS: m z calcd for C₉H₁₄F₃N₆O; 279.2; found 279.10 ([M+]).

### 8. Preparation of 3-bromopropan-1-amine hydro bromide (SOPL-WLS-94-07)

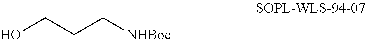 [SMILES: CC(C)(C)OC(=O)NCCCO]

To a stirred solution of (SM-2) (30 g, 0.4 mol) in DCM (1.5 L, 50 vol.) was added triethylamine (109.18 g, 0.8 mol.) dropwise at 0° C. and stirred for 20 mints. Then Boc-anhydride (100.9 mL, 0.44 mol) was added and stirred at rt for 24 h. Progress of the reaction was monitored by TLC. Above reaction was diluted with sat.NH₄Cl solution (300 mL) and extracted with DCM (2×300 mL), dried over Na₂SO₄and concentrated under vacuum to afford a light yellow liquid (SOPL-WLS-94-07) (60 g, crude). TLC Mobile phase details: 10% MeOH in DCM. ¹H NMR (400 MHz, DMSO-d₆): δ in ppm=6.72 (t, 1H, J1=5.1 Hz), 4.36 (t, 1H, J1=5.2 Hz), 3.39 (dd, 2H, J1=11.6 Hz, J2=6.3 Hz), 2.96 (q, 1H, J1=6.6 Hz), 1.52 (m, 2H), 1.37 (s, 9H). MS: m z calcd for C₃H₆Br, 175.2; found 75.91 ([M⁺−100]).

### 9. Preparation of tert-butyl (3-bromopropyl)carbamate (SOPL-WLS-94-08)

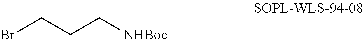 [SMILES: CC(C)(C)OC(=O)NCCCBr]

To a stirred solution of (SOPL-WLS-94-07) (60 g, 0.3429 mol) in DCM (1.2 L, 40 vol.) was added triphenylphosphine (134.6 g, 0.5143 mol.) and stirred at rt for 30 mins. Then the mixture was cool to 0° C. and carbon tetrabromide (170 g, 0.5143 mol) was added portion-wise. The mixture was further stirred at rt for 16 h. Progress of the reaction was monitored by TLC. Above mixture was concentrated under reduced pressure. The crude was purified by column chromatography over silica-gel (230-400 mesh) eluted in 20% EtOAC/Hexane to afford a pale yellow oil. (SOPL-WLS-94-08) (45 g, 45%). TLC Mobile phase details: 40% EtOAC/Hexane. ¹H NMR (400 MHz, DMSO-d₆): δ in ppm=6.89 (s, 1H), 3.50 (t, 2H, J1=6.5 Hz), 3.02, (q, 2H, J1=6.4 Hz), 1.90 (m, 2H), 1.37 (s, 9H). MS: m z calcd for C₈H₁₆BrNO₂, 238.1; found 139.84 ([M-99])

What is claimed is:

1. A double-stranded RNAi (dsRNAi) agent comprising a guide strand and a passenger strand wherein:
a) the guide strand is complementary or substantially complementary to a target RNA sequence, the guide strand comprises a backbone phosphoryl guanidine chiral center in the Sp configuration between the +3 nucleotide and the immediately downstream (+4) nucleotide, i.e., in the 3′ direction and further comprises:
i. backbone phosphorothioate chiral centers in Sp configuration between the 3′ terminal nucleotide and the penultimate (N-1) nucleotide and as between the penultimate (N-1) nucleotide and the immediately upstream (N-2) nucleotide;
ii. backbone phosphorothioate chiral centers in Rp, Sp, or alternating configurations between the 5′ terminal (+1) nucleotide and the immediately downstream (+2) nucleotide and between the +2 nucleotide and the immediately downstream (+3) nucleotide;
iii. one or more backbone phosphorothioate chiral centers in Rp or Sp configuration upstream of backbone phosphorothioate chiral centers in Sp configuration between the 3′ terminal nucleotide and the penultimate (N-1) nucleotide and as between the penultimate (N-1) nucleotide and the immediately upstream (N-2) nucleotide;
iv. one or more backbone phosphorothioate chiral centers in Rp or Sp configuration between the 5′ terminal (+1) nucleotide and the immediately downstream (+2) nucleotide; and/or between the (+2) nucleotide and the immediately downstream (+3) nucleotide;
v. one or more backbone phosphorothioate chiral centers in Rp or Sp configuration where linkage occurs between any two adjacent nucleotides between the penultimate 3′ (N-1) nucleotide of the guide strand, where N is the 3′ terminal nucleotide, and the upstream N-10 nucleotide;
vi. one or more backbone phosphoryl guanidine chiral center in the Sp configuration between the +7 and the immediately downstream (+8), i.e., in the 3′ direction; and/or
vii. a 5′ terminal modification;
b) the guide strand comprises one or more Rp, Sp, or stereorandom non-negatively charged internucleotidic linkage occurs between any two adjacent nucleotides between the second (+2) nucleotide relative to the 5′ terminal nucleotide of the guide strand and the penultimate 3′ (N-1) nucleotide of the guide strand, where N is the 3′ terminal nucleotide;
c) the guide strand comprises a 2′ modification, of the 3′ nucleotide of a nucleotide pair linked by an Rp, Sp, or stereorandom non-negatively charged internucleotidic linkage;
d) the guide strand comprises an Rp, Sp, or stereorandom non-negatively charged internucleotidic linkage between the tenth (+10) and eleventh (+11) nucleotides, relative to the 5′ terminal nucleotide;
e) the guide strand comprises an Rp, Sp, or stereorandom non-negatively charged internucleotidic linkage between the seventh (+7) and eighth (+8) nucleotides, relative to the 5′ terminal nucleotide;
f) the guide strand comprises one or more backbone phosphorothioate chiral centers in Rp or Sp configuration where linkage occurs between any two adjacent nucleotides between the penultimate 3′ nucleotide of the guide strand, where N is the 3′ terminal nucleotide, and the upstream N-10 nucleotide;
g) a passenger strand comprises one or more Rp, Sp, or stereorandom non-negatively charged internucleotidic linkage occurs upstream, i.e., in the 5′ direction, relative to the central nucleotide of the passenger strand;
h) the passenger strand comprises one or more Rp, Sp, or stereorandom non-negatively charged internucleotidic linkage occurs downstream, i.e., in the 3′ direction, relative to the central nucleotide of the passenger strand;
i) a passenger strand where one or more backbone phosphorothioate chiral centers in Rp or Sp configuration internucleotidic linkage occurs upstream, i.e., in the 5′ direction, relative to the central nucleotide of the passenger strand;
j) a passenger strand where one or more backbone phosphorothioate chiral centers in Rp or Sp configuration internucleotidic linkage internucleotidic linkage occurs downstream, i.e., in the 3′ direction, relative to the central nucleotide of the passenger strand;
k) a passenger strand in combination with one or more of the aforementioned guide strands, comprising one or more modified sugars between the 5′ terminal (+1) nucleotide and the penultimate (N-1) nucleotide;
l) the passenger strand comprises one or both of:
i. 0-n Rp, Sp, or stereorandom non-negatively charged internucleotidic linkages, where n is about 1 to 49;
ii. one or more backbone chiral centers in Rp or Sp configuration;
iii. one or more backbone phosphoryl guanidine chiral centers in the Rp configuration between the +7 nucleotide and the immediately downstream (+8) nucleotide, i.e., in the 3′ direction;
iv. one or more backbone phosphoryl guanidine chiral centers in the Rp configuration between the +15 nucleotide and the immediately downstream (+16) nucleotide, i.e., in the 3′ direction; and/or
v. backbone phosphorothioate chiral centers in the Sp configuration between the 5′ terminal (+1) nucleotide and the immediately downstream, i.e., in the 3′ direction, (+2) nucleotide and between the 3′ terminal nucleotide and the penultimate (N-1) nucleotide;
m) each strand of the dsRNAi agent independently has a length of about 15 to about 49 nucleotides; and/or
n) the dsRNAi is capable of directing target-specific RNA interference.

2. A chirally controlled oligonucleotide composition comprising double stranded oligonucleotides wherein the guide and passenger strands of the double stranded oligonucleotides are independently characterized by:
a) a common base sequence and length;
b) a common pattern of backbone linkages; and
c) a common pattern of backbone chiral centers;
which composition is chirally controlled in that it is enriched, relative to a substantially racemic preparation of guide strands having the same common base sequence and length, for oligonucleotides having a common pattern of chiral centers; and
a) wherein the guide strands are complementary or substantially complementary to a target RNA sequence, the guide strands comprise a backbone phosphoryl guanidine chiral center in the Sp configuration between the +3 nucleotide and the immediately downstream (+4) nucleotide, i.e., in the 3′ direction and further comprise:
i. backbone phosphorothioate chiral centers in Sp configuration between the 3′ terminal nucleotide and the penultimate (N-1) nucleotide and as between the penultimate (N-1) nucleotide and the immediately upstream (N-2) nucleotide,
ii. backbone phosphorothioate chiral centers in Rp, Sp, or alternating configurations between the 5′ terminal (+1) nucleotide and the immediately downstream (+2) nucleotide and between the +2 nucleotide and the immediately downstream (+3) nucleotide;
iii. one or more backbone phosphorothioate chiral centers in Rp or Sp configuration upstream of backbone phosphorothioate chiral centers in Sp configuration between the 3′ terminal nucleotide and the penultimate (N-1) nucleotide and as between the penultimate (N-1) nucleotide and the immediately upstream (N-2) nucleotide;
iv. one or more backbone phosphorothioate chiral centers in Rp or Sp configuration between the 5′ terminal (+1) nucleotide and the immediately downstream (+2) nucleotide; and/or between the (+2) nucleotide and the immediately downstream (+3) nucleotide; and/or backbone phosphorothioate chiral centers in the Sp configuration between the 5′ terminal (+1) nucleotide and the immediately downstream, i.e., in the 3′ direction, (+2) nucleotide and between the 3′ terminal nucleotide and the penultimate (N-1) nucleotide;
v. one or more backbone phosphoryl guanidine chiral center in the Sp configuration between the +7 and the immediately downstream (+8), i.e., in the 3′ direction; and/or
vi. a 5′ terminal modification;
b) the guide strand comprises one or more Rp, Sp, or stereorandom non-negatively charged internucleotidic linkage occurs between any two adjacent nucleotides between the second (+2) nucleotide relative to the 5′ terminal nucleotide of the guide strand and the penultimate 3′ (N-1) nucleotide of the guide strand, where N is the 3′ terminal nucleotide;
c) the guide strand comprises a 2′ modification, of the 3′ nucleotide of a nucleotide pair linked by an Rp, Sp, or stereorandom non-negatively charged internucleotidic linkage;
d) the guide strand comprises an Rp, Sp, or stereorandom non-negatively charged internucleotidic linkage between the tenth (+10) and eleventh (+11) nucleotides, relative to the 5′ terminal nucleotide;
e) the guide strand comprises an Rp, Sp, or stereorandom non-negatively charged internucleotidic linkage between the seventh (+7) and eighth (+8) nucleotides, relative to the 5′ terminal nucleotide;
f) the guide strand comprises one or more backbone phosphorothioate chiral centers in Rp or Sp configuration where linkage occurs between any two adjacent nucleotides between the penultimate 3′ nucleotide of the guide strand, where N is the 3′ terminal nucleotide, and the upstream N-10 nucleotide;
g) a passenger strand comprises one or more Rp, Sp, or stereorandom non-negatively charged internucleotidic linkage occurs upstream, i.e., in the 5′ direction, relative to the central nucleotide of the passenger strand;
h) the passenger strand comprises one or more Rp, Sp, or stereorandom non-negatively charged internucleotidic linkage occurs downstream, i.e., in the 3′ direction, relative to the central nucleotide of the passenger strand;
i) a passenger strand where one or more backbone phosphorothioate chiral centers in Rp or Sp configuration internucleotidic linkage occurs upstream, i.e., in the 5′ direction, relative to the central nucleotide of the passenger strand;
j) a passenger strand where one or more backbone phosphorothioate chiral centers in Rp or Sp configuration internucleotidic linkage internucleotidic linkage occurs downstream, i.e., in the 3′ direction, relative to the central nucleotide of the passenger strand;
k) a passenger strand in combination with one or more of the aforementioned guide strands, comprising one or more modified sugars between the 5′ terminal (+1) nucleotide and the penultimate (N-1) nucleotide;
l) the passenger strands comprise one or both of:
i. 0-n Rp, Sp, or stereorandom non-negatively charged internucleotidic linkages, where n is about 1 to 49;
ii. one or more backbone chiral centers in Rp or Sp configuration;
iii. one or more backbone phosphoryl guanidine chiral centers in the Rp configuration between the +7 nucleotide and the immediately downstream (+8) nucleotide, i.e., in the 3′ direction;
iv. one or more backbone phosphoryl guanidine chiral centers in the Rp configuration between the +15 nucleotide and the immediately downstream (+16) nucleotide, i.e., in the 3′ direction; and/or
v. backbone phosphorothioate chiral centers in the Sp configuration between the 5′ terminal (+1) nucleotide and the immediately downstream, i.e., in the 3′ direction, (+2) nucleotide and between the 3′ terminal nucleotide and the penultimate (N-1) nucleotide;
m) the guide and passenger strands have a length of about 15 to about 49 nucleotides; and/or
n) the guide and passenger strands are capable of directing target-specific RNA interference.

3. The double stranded oligonucleotide of claim 1 or the composition of claim 2, wherein the guide strand comprises backbone phosphorothioate chiral centers in Sp configuration between the 3′ terminal nucleotide and the penultimate (N-1) nucleotide and as between the penultimate (N-1) nucleotide and the immediately upstream (N-2) nucleotide, and the passenger strand comprises 0-n Rp, Sp, or stereorandom non-negatively charged internucleotidic linkages, where n is about 1 to 49.

4. The double stranded oligonucleotide of claim 1 or the composition of claim 2, wherein the guide strand comprises backbone phosphorothioate chiral centers in Rp, Sp, or alternating configurations between the 5′ terminal (+1) nucleotide and the immediately downstream (+2) nucleotide and between the +2 nucleotide and the immediately downstream (+3) nucleotide, and the passenger strand comprises 0-n Rp, Sp, or stereorandom non-negatively charged internucleotidic linkages, where n is about 1 to 49.5. The double stranded oligonucleotide of claim 1 or the composition of claim 2, wherein the guide strand comprises one or more backbone phosphorothioate chiral centers in Rp or Sp configuration where linkage occurs between any two adjacent nucleotides between the penultimate 3′ nucleotide of the guide strand, where N is the 3′ terminal nucleotide, and the upstream N-10 nucleotide, and the passenger strand comprises 0-n Rp, Sp, or stereorandom non-negatively charged internucleotidic linkages, where n is about 1 to 49.

6. The double stranded oligonucleotide of claim 1 or the composition of claim 2, wherein the guide strand comprises one or more backbone phosphorothioate chiral centers in Rp or Sp configuration upstream of backbone phosphorothioate chiral centers in Sp configuration between the 3′ terminal nucleotide and the penultimate (N-1) nucleotide and as between the penultimate (N-1) nucleotide and the immediately upstream (N-2) nucleotide, and the passenger strand comprises 0-n Rp, Sp, or stereorandom non-negatively charged internucleotidic linkages, where n is about 1 to 49.

7. The double stranded oligonucleotide of claim 1 or the composition of claim 2, wherein the guide strand comprises one or more Rp, Sp, or stereorandom non-negatively charged internucleotidic linkage occurs between any two adjacent nucleotides between the second (+2) nucleotide relative to the 5′ terminal nucleotide of the guide strand and the penultimate 3′ (N-1) nucleotide of the guide strand, where N is the 3′ terminal nucleotide, and the passenger strand comprises 0-n Rp, Sp, or stereorandom non-negatively charged internucleotidic linkages, where n is about 1 to 49.

8. The double stranded oligonucleotide of claim 1 or the composition of claim 2, wherein the guide strand comprises backbone phosphorothioate chiral centers in Sp configuration between the 3′ terminal nucleotide and the penultimate (N-1) nucleotide and as between the penultimate (N-1) nucleotide and the immediately upstream (N-2) nucleotide, and the passenger strand comprises one or more backbone chiral centers in Rp or Sp configuration.

9. The double stranded oligonucleotide of claim 1 or the composition of claim 2, wherein the guide strand comprises backbone phosphorothioate chiral centers in Rp, Sp, or alternating configurations between the 5′ terminal (+1) nucleotide and the immediately downstream (+2) nucleotide and between the +2 nucleotide and the immediately downstream (+3) nucleotide, and the passenger strand comprises one or more backbone chiral centers in Rp or Sp configuration.

10. The double stranded oligonucleotide of claim 1 or the composition of claim 2, wherein the guide strand comprises one or more backbone phosphorothioate chiral centers in Rp or Sp configuration where linkage occurs between any two adjacent nucleotides between the penultimate 3′ nucleotide of the guide strand, where N is the 3′ terminal nucleotide, and the upstream N-10 nucleotide, and the passenger strand comprises one or more backbone chiral centers in Rp or Sp configuration.

11. The double stranded oligonucleotide of claim 1 or the composition of claim 2, wherein the guide strand comprises a backbone phosphoryl guanidine chiral center in the Sp configuration between the +7 and the immediately downstream (+8), i.e., in 3′ the direction, and the passenger strand comprises 0-n Rp, Sp, or stereorandom non-negatively charged internucleotidic linkages, where n is about 1 to 49.

12. The double stranded oligonucleotide of claim 1 or the composition of claim 2, wherein the guide strand comprises a backbone phosphoryl guanidine chiral center in the Sp configuration between the +7 and the immediately downstream (+8), i.e., in 3′ the direction, and the passenger strand comprises one or more backbone chiral centers in Rp or Sp configuration.

13. The double stranded oligonucleotide of claim 1 or the composition of claim 2, wherein the guide strand comprises one or more backbone phosphorothioate chiral centers in Rp or Sp configuration upstream of backbone phosphorothioate chiral centers in Sp configuration between the 3′ terminal nucleotide and the penultimate (N-1) nucleotide and as between the penultimate (N-1) nucleotide and the immediately upstream (N-2) nucleotide, and the passenger strand comprises one or more backbone chiral centers in Rp or Sp configuration.

14. The double stranded oligonucleotide of claim 1 or the composition of claim 2, wherein the guide strand comprises one or more backbone phosphorothioate chiral centers in Rp or Sp configuration between the 5′ terminal (+1) nucleotide and the immediately downstream (+2) nucleotide; and/or between the (+2) nucleotide and the immediately downstream (+3) nucleotide.

15. The double stranded oligonucleotide of claim 1 or the composition of claim 2, wherein the guide strand comprises a 5′ terminal modification selected from: 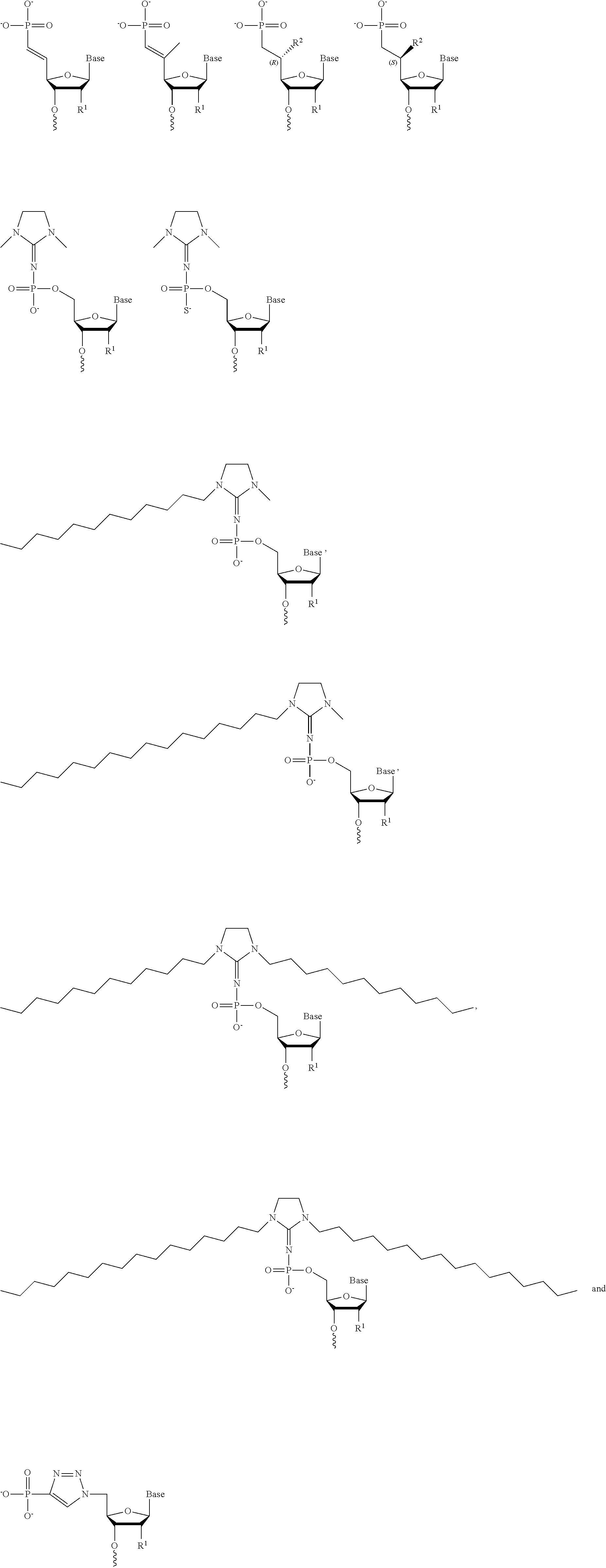
Base: A, C, G, T, U, abasic, and modified nucleobases;
R¹: H, OH, O-alkyl, O-Me, F, MOE, LNA bridge to the 4′ position, BNA bridge to the 4′ position.
R²: alkyl, methyl, ethyl, isopropyl, propyl, cyclohexyl, benzyl, phenyl, tolyl, xylyl, aryl, or arene group.

16. The double stranded oligonucleotide or composition of claim 13 wherein the guide strand comprises a 5′ terminal modification selected from 5′ MeP modifications and 5′ Trizole-P modifications.

17. The double stranded oligonucleotide or composition of claim 14 wherein the 5′ MeP modification is

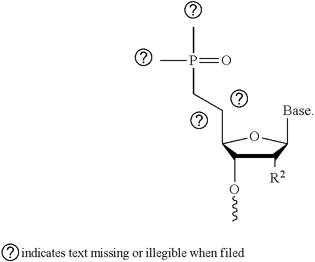

18. The double stranded oligonucleotide or composition of claim 15, comprising a backbone phosphorothioate chiral center in Sp configuration between the 5′ terminal (+1) nucleotide and the immediately downstream (+2) nucleotide, and a backbone phosphorothioate chiral center in the Rp configuration between the +2 nucleotide and the immediately downstream (+3) nucleotide.

19. The double stranded oligonucleotide of claim 1 or the composition of claim 2, wherein the guide strand comprises one or more Rp, Sp, or stereorandom non-negatively charged internucleotidic linkage occurs between any two adjacent nucleotides between the second (+2) nucleotide relative to the 5′ terminal nucleotide of the guide strand and the penultimate 3′ (N-1) nucleotide of the guide strand, where N is the 3′ terminal nucleotide, and the passenger strand comprises one or more backbone chiral centers in Rp or Sp configuration.

20. The double stranded oligonucleotide of claim 1 or the composition of claim 2, wherein the guide strand comprises backbone phosphorothioate chiral centers in Sp configuration between the 3′ terminal nucleotide and the penultimate (N-1) nucleotide and as between the penultimate (N-1) nucleotide and the immediately upstream (N-2) nucleotide, and the passenger strand comprises 0-n Rp, Sp, or stereorandom non-negatively charged internucleotidic linkages, where n is about 1 to 49 and one or more backbone chiral centers in Rp or Sp configuration.

21. The double stranded oligonucleotide of claim 1 or the composition of claim 2, wherein the guide strand comprises backbone phosphorothioate chiral centers in Rp, Sp, or alternating configurations between the 5′ terminal (+1) nucleotide and the immediately downstream (+2) nucleotide and between the +2 nucleotide and the immediately downstream (+3) nucleotide, and the passenger strand comprises 0-n Rp, Sp, or stereorandom non-negatively charged internucleotidic linkages, where n is about 1 to 49 and one or more backbone chiral centers in Rp or Sp configuration.

22. The double stranded oligonucleotide of claim 1 or the composition of claim 2, wherein the guide strand comprises one or more backbone phosphorothioate chiral centers in Rp or Sp configuration upstream of backbone phosphorothioate chiral centers in Sp configuration between the 3′ terminal nucleotide and the penultimate (N-1) nucleotide and as between the penultimate (N-1) nucleotide and the immediately upstream (N-2) nucleotide, and the passenger strand comprises 0-n Rp, Sp, or stereorandom non-negatively charged internucleotidic linkages, where n is about 1 to 49 and one or more backbone chiral centers in Rp or Sp configuration.

23. The double stranded oligonucleotide of claim 1 or the composition of claim 2, wherein the guide strand comprises one or more Rp, Sp, or stereorandom non-negatively charged internucleotidic linkage occurs between any two adjacent nucleotides between the second (+2) nucleotide relative to the 5′ terminal nucleotide of the guide strand and the penultimate 3′ (N-1) nucleotide of the guide strand, where N is the 3′ terminal nucleotide, and the passenger strand comprises 0-n non-negatively charged internucleotidic linkages, where n is about 1 to 49 and one or more backbone chiral centers in Rp or Sp configuration.

24. The double stranded oligonucleotide or composition of any one of the preceding claims, wherein the Rp, Sp, or stereorandom non-negatively charged backbone internucleotidic linkages have neutral charge.

25. The double stranded oligonucleotide or composition of claim 24, wherein the neutral backbone internucleotidic linkages is

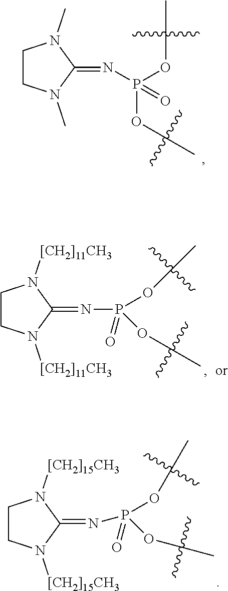

26. The double stranded oligonucleotide or composition of claim 25, wherein the guide strand comprises a linkage having the following structure 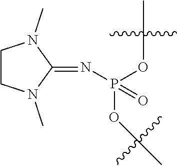 [SMILES: *OP(=O)(N=C1N(C)CCN1C)O*]
between the third (+3) and fourth (+4) nucleotides of the guide strand, between the tenth (+10) and eleventh (+11) nucleotides of the guide strand, or both.

27. The double stranded oligonucleotide or composition of claim 25, wherein the guide strand comprises a linkage having the following structure 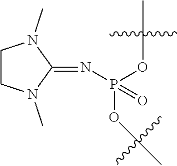 [SMILES: *OP(=O)(N=C1N(C)CCN1C)O*]
between the third (+3) and fourth (+4) nucleotides of the guide strand, between the seventh (+7) and eighth (+8) nucleotides of the guide strand, between the tenth (+10) and eleventh (+11) nucleotides of the guide strand, between the eighteenth (+18) and nineteenth (+19) nucleotides of the guide strand, or combinations thereof.

28. The double stranded oligonucleotide or composition of claim 25, wherein the passenger strand comprises a linkage having the following structure 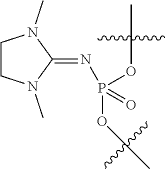 [SMILES: *OP(=O)(N=C1N(C)CCN1C)O*]
5′ to the central nucleotide of the passenger strand, 3′ to the central nucleotide of the passenger strand, or both.

29. The composition of claim 2, where the guide and passenger strands in the composition that independently share a common base sequence, a common pattern of base modification, a common pattern of sugar modification, and/or a common pattern of internucleotidic linkages are at least 90% f all the guide and passenger strands in the composition.

30. The double stranded oligonucleotide or composition of any of the preceding claims, wherein the double stranded oligonucleotide comprises a carbohydrate moiety connected at a nucleoside or an internucleotide linkage, optionally through a linker.

31. The double stranded oligonucleotide or composition of any of the preceding claims, wherein the double stranded oligonucleotide comprises a lipid moiety connected to the double stranded oligonucleotide at a nucleoside or an internucleotide linkage, optionally through a linker.

32. The double stranded oligonucleotide or composition of any of the preceding claims, wherein one or both strands of the double stranded oligonucleotide comprises a target moiety connected at a nucleobase, optionally through a linker.

33. The double stranded oligonucleotide or composition of any one of the preceding claims, wherein at least 20%, 25%, 30%, 35%, 40%, 45%, 50%, 55%, 60%, 65%, 70%, 75%, 80%, 85%, 90% or 95% of the internucleotidic linkages of the double stranded oligonucleotide are independently chiral internucleotidic linkages.

34. The double stranded oligonucleotide or composition of any one of the preceding claims, wherein at least 3%, 5%, 10%, 15%, 20%, 25%, 30%, 35%, 40%, 45%, 50%, 55%, 60%, 65%, 70%, 75%, 80%, 85%, 90%, 95%, or 97% f the nucleotidic units of the double stranded oligonucleotide independently comprise a 2′-substitution.

35. The double stranded oligonucleotide or composition of any one of the preceding claims, wherein a modified sugar of the oligonucleotide comprises a 2′-F modification, 2′-OH modification, 2′-OMe modification, 2′-O—C16 lipid modification, 5′-alkyl modification, 2′-MOE modification, DNA, LNA, UNA, GNA, or a Homo-DNA.

36. The double stranded oligonucleotide or composition of any one of the preceding claims, wherein a modified sugar of the oligonucleotide is at one position or a plurality of positions.

37. The double stranded oligonucleotide or composition of any one of the preceding claims, wherein a modified sugar of the oligonucleotide is at one or more of: (a) position +1; (b) position +2; (c) position +3; (d) position +4; (e) position +5; and (f) position +6.

38. The double stranded oligonucleotide or composition of claims 35-37, wherein a modified sugar of the oligonucleotide is at position +4 and wherein the modified sugar of the oligonucleotide is a 2′-F modification.

39. The double stranded oligonucleotide or composition of any one of the preceding claims, wherein a 2′-substitution of the oligonucleotide is -L-, wherein L connects C₂ and C₄ of the sugar unit.

40. The double stranded oligonucleotide or composition of any one of the preceding claims, wherein at least 3%, 5%, 10%, 15%, 20%, 25%, 30%, 35%, 40%, 45%, 50%, 55%, 60%, 65%, 70%, 75%, 80%, 85%, 90%, 95%, or 97% f the nucleotidic units of the double stranded oligonucleotide comprise no 2′-substitution.

41. The double stranded oligonucleotide or composition of any one of the preceding claims, wherein the guide strand comprises a target-binding sequence that is completely complementary to a target sequence, wherein the target-binding sequence has a length of at least 10, 11, 12, 13, 14, 15, 16, 17, 18, 19 or 20 bases, wherein each base is optionally substituted adenine, cytosine, guanosine, thymine, or uracil, and wherein the target sequence comprises one or more allelic sites, wherein an allelic site is a SNP or a mutation.

42. The double stranded oligonucleotide or composition of any one of the preceding claims, wherein the target sequence comprises two SNPs.

43. The double stranded oligonucleotide or composition of any one of the preceding claims, wherein the target sequence comprises an allelic site and the target-binding sequence is completely complementary to the target sequence of a disease-associated allele but not that of an allele less associated with the disease.

44. The double stranded oligonucleotide or composition of any one of the preceding claims, wherein
the double stranded oligonucleotide comprises a guide strand that binds with a transcript of a target nucleic acid sequence for which a plurality of alleles exist within a population, each of which contains a specific nucleotide characteristic sequence element that defines the allele relative to other alleles of the same target nucleic acid sequence,
wherein the base sequence of the guide strand is or comprises a sequence that is complementary to the characteristic sequence element that defines a particular allele, and
the guide strand being characterized in that, when it is contacted with a cell comprising transcripts of target nucleic acid sequence, it shows suppression of transcripts of the particular allele, or a protein encoded thereby, at a level that is greater than a level of suppression observed for another allele of the same nucleic acid sequence.

45. The double stranded oligonucleotide or composition of any one of the preceding claims, wherein the passenger strand comprises:
an Sp backbone phosphorothioate chiral center between the 5′ terminal (+1) nucleotide and the immediately downstream (+2) nucleotide; and
an Sp backbone phosphorothioate chiral center between the penultimate (N-1) nucleotide and the 3′ terminal (N) nucleotide.

46. A method for reducing level and/or activity of a transcript or a protein encoded thereby, comprising administering to a cell expressing the transcript a double stranded oligonucleotide or a composition of any one of the preceding claims, wherein the guide strand of double stranded oligonucleotide or composition comprises a targeting-binding sequence that is completely complementary to a target sequence in the transcript.

47. The method of claim 41 wherein the cell is an immune cell, a blood cell, a cardiac cell, a lung cell, an optic cell, a muscle cell, a liver cell, a kidney cell, a brain cell, a cell of the central nervous system, or a cell of the peripheral nervous system.

48. A method for allele-specific suppression of a transcript from a nucleic acid sequence for which a plurality of alleles exist within a population, each of which contains a specific nucleotide characteristic sequence element that defines the allele relative to other alleles of the same target nucleic acid sequence, the method comprising steps of:
contacting a sample comprising transcripts of the target nucleic acid sequence with a double stranded oligonucleotide or a composition of any one of the preceding claims,
wherein the guide strand of the double stranded oligonucleotide or composition comprises a targeting-binding sequence that is identical or completely complementary to a target sequence in the nucleic acid sequence, which target sequence comprises a characteristic sequence element that defines a particular allele, and
wherein when the guide strand of the double stranded oligonucleotide or composition is contacted with a cell comprising transcripts of both the target allele and another allele of the same nucleic acid sequence, transcripts of the particular allele are suppressed at a greater level than a level of suppression observed for another allele of the same nucleic acid sequence.

49. A method for allele-specific suppression of a transcript from a nucleic acid sequence for which a plurality of alleles exist within a population, each of which contains a specific nucleotide characteristic sequence element that defines the allele relative to other alleles of the same target nucleic acid sequence, the method comprising steps of:
administering to a subject comprising transcripts of the target nucleic acid sequence with a double stranded oligonucleotide or a composition of any one of the preceding claims,
wherein the guide strand of the double stranded oligonucleotide or composition comprises a targeting-binding sequence that is identical or completely complementary to a target sequence in the nucleic acid sequence, which target sequence comprises a characteristic sequence element that defines a particular allele, and
wherein when the guide strand of the double stranded oligonucleotide or composition is contacted with a cell comprising transcripts of both the target allele and another allele of the same nucleic acid sequence, transcripts of the particular allele are suppressed at a greater level than a level of suppression observed for another allele of the same nucleic acid sequence.

50. The method of any one of claims 46-49, wherein when the oligonucleotide or oligonucleotide of the composition is contacted with a cell comprising transcripts of both the target allele and another allele of the same nucleic acid sequence, it shows suppression of transcripts of the particular allele at a level that is:
a) greater than when the composition is absent;
b) greater than a level of suppression observed for another allele of the same nucleic acid sequence; or
c) both greater than when the composition is absent, and greater than a level of suppression observed for another allele of the same nucleic acid sequence.

52. The method of claim 50 wherein the cell is an immune cell, a blood cell, a cardiac cell, a lung cell, an optic cell, a muscle cell, a liver cell, a kidney cell, a brain cell, a cell of the central nervous system, or a cell of the peripheral nervous system.

53. The method of any one of claims 46-52, wherein suppression of transcripts of the particular allele is at a level that is both greater than when the composition is absent, and greater than a level of suppression observed for another allele of the same nucleic acid sequence.
